(19) United States
(12) Patent Application Publication
(10) Pub. No.: US 2023/0391760 A1
(43) Pub. Date: Dec. 7, 2023

(54) HETEROCYCLIC GLP-1 AGONISTS

(71) Applicant: Gasherbrum Bio, Inc., South San Francisco, CA (US)

(72) Inventors: Qinghua MENG, Shanghai (CN); Weiqiang XING, Shanghai (CN); Xichen LIN, Shanghai (CN); Andrew JENNINGS, San Diego, CA (US)

(21) Appl. No.: 18/248,857

(22) PCT Filed: Oct. 13, 2021
(86) PCT No.: PCT/CN2021/123619

§ 371 Date: Apr. 12, 2023

(30) Foreign Application Priority Data
Oct. 13, 2020 (WO) PCT/CN2020/120605
Sep. 3, 2021 (WO) PCT/CN2021/116470

(51) Int. Cl. C07D 405/14; C07D 409/14; C07D 471/04; C07D 493/08; C07D 417/14; C07D 413/14; C07D 487/04; A61K 45/06

(52) U.S. Cl. CPC C07D 405/14; C07D 409/14; C07D 471/04; A61K 45/06; C07D 417/14; C07D 413/14; C07D 487/04; C07D 493/08

(57) ABSTRACT

This disclosure relates to GLP-1 agonists of Formula (I): including pharmaceutically acceptable salts and solvates thereof, and pharmaceutical compositions including the same.

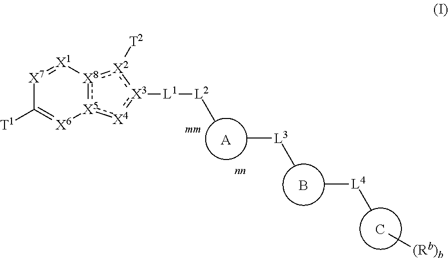

### CROSS REFERENCE TO RELATED APPLICATIONS

This application claims the benefit of International Patent Application Number PCT/CN2020/120605, filed on Oct. 13, 2020; and International Application Number PCT/CN2021/116470, filed on Sep. 3, 2021 each of which is incorporated herein by reference in its entirety.

### TECHNICAL FIELD

This disclosure relates to GLP-1 agonists, pharmaceutical compositions, and methods of use thereof.

### BACKGROUND

Incretin metabolic hormones, including glucagon-like peptide-1 (GLP-1) and glucose-dependent insulinotropic polypeptide (GIP), are important in the regulation of glucose homeostasis. Medicaments targeting this family of intestinal peptides, such as GLP-1 agonists, have been shown to suppress glucagon production, decrease gastric motility, and increase satiety.

Diabetes mellitus refers to a group of metabolic disorders characterized by persistent hyperglycemia. The most common form, type 2 diabetes mellitus (T2DM) is an acquired condition that accounts for more than 90% of diabetes cases. Typical onset occurs in obese or otherwise sedentary adults and begins with insulin resistance. Though lifestyle changes can be useful in management of this disorder, patients with T2DM may be required to take antidiabetic medications, including dipeptidyl peptidase-4 inhibitors, SGLT2 inhibitors, and sulfonylureas, among others.

In healthy individuals, the incretin hormones glucose-dependent insulinotropic polypeptide (GIP) and glucagon-like peptide 1 (GLP-1) provide tandem modulation of insulin secretory response to glucose ingestion. While this incretin effect is significantly diminished (if at all present) in cases of T2DM, GLP-1 retains insulinotropic properties, even as endocrine pancreatic response to GIP is effectively halted. As such, incretin mimetics and other GLP-1-based therapies can help stimulate insulin production in T2DM patients.

### SUMMARY

The present application describes heterocyclic GLP-1 agonists, as well as pharmaceutical compositions comprising the compounds disclosed herein. Also provided are methods for treating GLP-1-associated diseases, disorders, and conditions.

Accordingly, provided herein are compounds of Formula I:

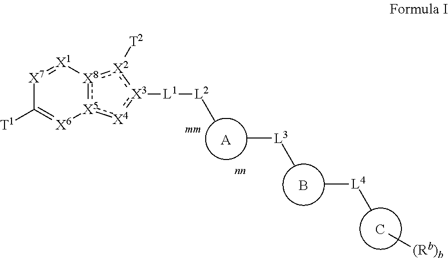

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

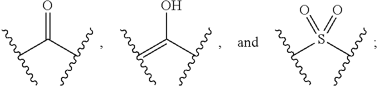

5- to 10-membered heterocycloalkyl, wherein the heterocycloalkyl comprises an endocyclic group selected from the group consisting of:

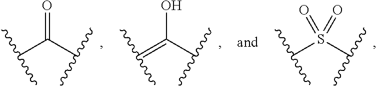

wherein the heterocycloalkyl is optionally substituted with 1-4 R^{v}; and
(C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 6-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T};
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

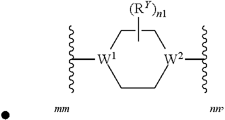

wherein n1 is 0, 1, or 2; W¹ is CR^{Y1} or N; and W² is CR or N;

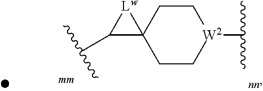

wherein W² is CR^{Y2} or N, L^{w} is (C₁-C₃)alkylene;
   phenylene optionally substituted with 1-4 R^{Y};
   5- to 6-membered heteroarylene optionally substituted with 1-3 R^{Y};
   partially unsaturated monocyclic (C₅-C₈)cycloalkylene optionally substituted with 1-4 R^{Y}; and
   partially unsaturated monocyclic 5- to 8-membered heterocycloalkylene optionally substituted with 1-4 R^{Y};
wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is a bond;
Ring B is selected from the group consisting of (B-I), (B-II), (B-III), (B-IV), and (B-V):

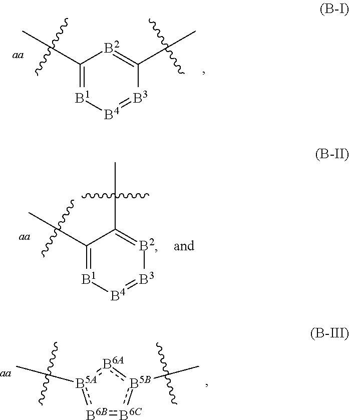

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR, NR, and N,
each [symbol] in (B-III) is independently a single bond or a double bond, provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

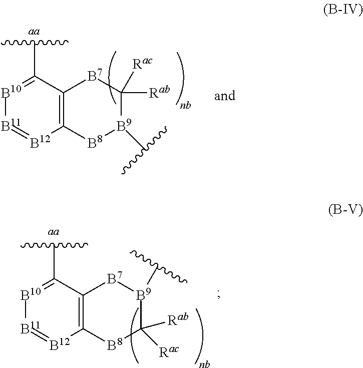

wherein aa represents the point of attachment to L³;
B⁷ and B⁸ are independently selected from the group consisting of: —O—, —NR^{N}—, and —C(R¹)₂—;
B is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

Also provided herein are compounds of Formula I:

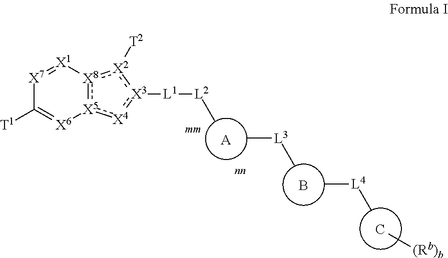

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

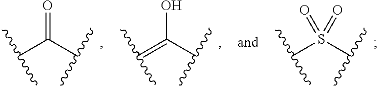

5- to 10-membered heterocycloalkyl, wherein the heterocycloalkyl comprises an endocyclic group selected from the group consisting of:

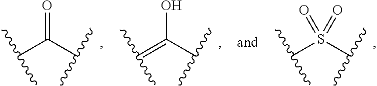

wherein the heterocycloalkyl is optionally substituted with 1-4 R^{v}; and
(C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring; or when q is 2 or 3, a pair of R^{x} on adjacent carbon atoms, taken together form a double bond between the adjacent carbon atoms;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 10-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, (C₃-C₈)cycloalkyl, —O(C₁-C₆)alkylene-O(C₁-C₆)alkyl, —OH, —N(R^{s})₂ 3- to 8-membered heterocycloalkyl, —CO₂H, (C₁-C₆)hydroxyalkyl, (C₁-C₆)alkyl-S(O)₁₋₂—(C₁-C₆)alkyl, (C₁-C₆)alkyl-C(═O)OH, —S(O)₀₋₂—C₁-C₆ alkyl, (C₁-C₆)alkenyl, —P(═O)R^{z1}R^{z2}, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T};
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, —C(═O)C₁-C₆ alkyl, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

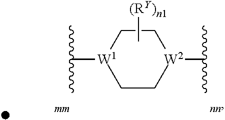

wherein n1 is 0, 1, or 2; W¹ is CR^{Y1} or N; and W² is CR^{Y2} or N;

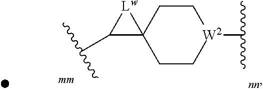

wherein W² is CR^{Y2} or N, L^{w} is (C₁-C₃)alkylene;
   phenylene optionally substituted with 1-4 R^{Y};
5- to 6-membered heteroarylene optionally substituted with 1-3 R^{Y};
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y};
wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is a bond;
Ring B is selected from the group consisting of: (B-I), (B-II), (B-III), (B-IV), and (B-V):

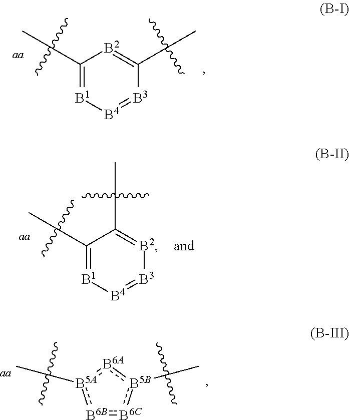

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

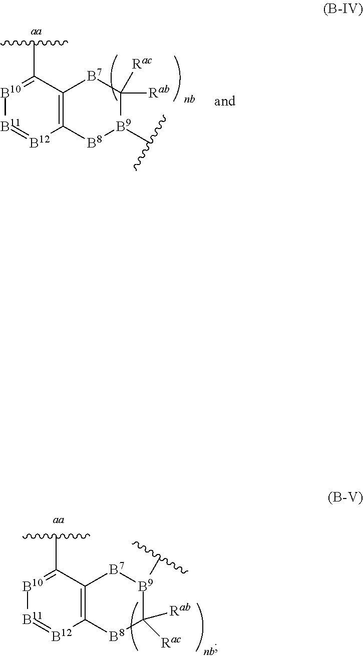

wherein aa represents the point of attachment to L³;
B⁷ and B⁸ are independently selected from the group consisting of: —O—, —NR^{N}—, and —C(R¹)₂—;
B is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

Further provided herein are compounds of A Formula I:

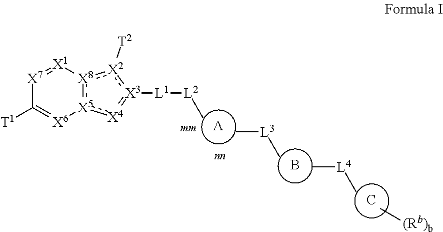

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

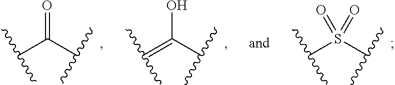

5- to 10-membered heterocycloalkyl, wherein the heterocycloalkyl comprises an endocyclic group selected from the group consisting of:

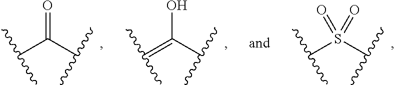

wherein the heterocycloalkyl is optionally substituted with 1-4 R^{v}; and
(C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring; or when q is 2 or 3, a pair of R^{x} on adjacent carbon atoms, taken together form a double bond between the adjacent carbon atoms;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 10-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, (C₃-C₈)cycloalkyl, —O(C₁-C₆)alkylene-O(C₁-C₆)alkyl, —OH, —N(R^{s})₂ 3- to 8-membered heterocycloalkyl, —CO₂H, (C₁-C₆)hydroxyalkyl, (C₁-C₆)alkyl-S(O)₁₋₂—(C₁-C₆)alkyl, (C₁-C₆)alkyl-C(═O)OH, —S(O)₀₋₂—C₁-C₆ alkyl, (C₁-C₆)alkenyl, —P(═O)R^{z1}R^{z2}, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T};
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, —C(═O)C₁-C₆ alkyl, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

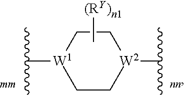

wherein n1 is 0, 1, or 2; W¹ is CR^{Y1} or N; and W² is CR^{Y2} or N;

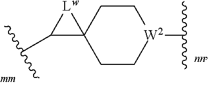

wherein W² is CR^{Y2} or N, L^{w} is (C₁-C₃)alkylene;
   phenylene optionally substituted with 1-4 R^{Y};
   5- to 6-membered heteroarylene optionally substituted with 1-3 R^{Y};
   partially unsaturated monocyclic (C₅-C₈)cycloalkylene optionally substituted with 1-4 R^{Y}; and
   partially unsaturated monocyclic 5- to 8-membered heterocycloalkylene optionally substituted with 1-4 R^{Y};
wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is a bond;
Ring B is selected from the group consisting of: (B-I), (B-II), (B-III), (B-IV), and (B-V):

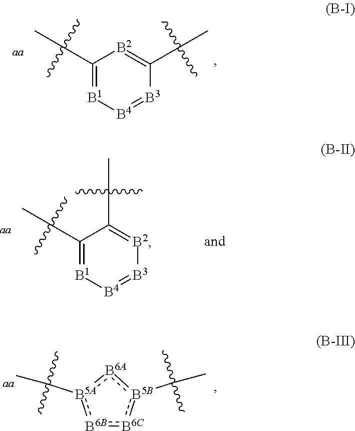

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

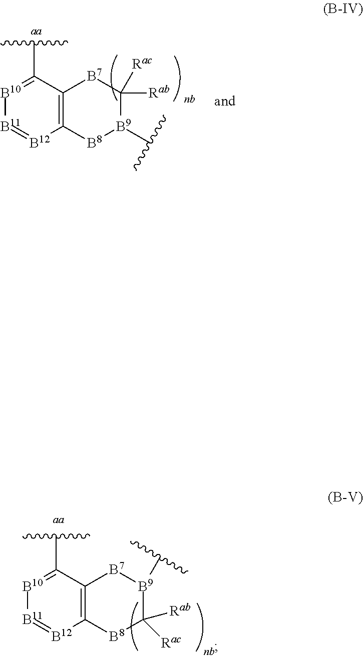

wherein aa represents the point of attachment to L³;
B⁷ and B⁸ are independently selected from the group consisting of: —O—, —NR^{N}—, and —C(R¹)₂—;
B is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, CN, —C(O)NH₂, —CONH(C₁-C₃ alkyl) and C(O)N(C₁-C₃)alky)₂; and
b is an integer selected from 0-3.

Also provided herein are pharmaceutical compositions comprising a compound of Formula I, or a pharmaceutically acceptable salt or solvate thereof, and a pharmaceutically acceptable excipient.

Also provided herein are methods for treating type 2 diabetes mellitus in a patient in need thereof, the methods comprising administering to the patient a therapeutically effective amount of a compound of Formula I, or a pharmaceutically acceptable salt or solvate thereof, or a pharmaceutical composition thereof.

Also provided herein are methods for treating type 2 diabetes mellitus in a patient, the methods comprising administering to a patient identified or diagnosed as having type 2 diabetes mellitus a therapeutically effective amount of a compound of Formula I, or a pharmaceutically acceptable salt or solvate thereof, or a pharmaceutical composition thereof.

Also provided herein are methods for treating diabetes mellitus in a patient, the methods comprising determining that the patient has type 2 diabetes mellitus; and administering to the patient a therapeutically effective amount of a compound of Formula I, or a pharmaceutically acceptable salt or solvate thereof, or a pharmaceutical composition thereof. In some embodiments, the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof. In some embodiments, the level of HbA1c is greater than or about 6.5%. In some embodiments, the level of fasting plasma glucose is greater than or about 126 mg/dL. In some embodiments, the level of non-fasting plasma glucose is greater than or about 200 mg/dL.

In some embodiments, the methods further comprise obtaining a sample from the patient. In some embodiments, the sample is a body fluid sample. In some embodiments, the patient is about 40 to about 70 years old and is overweight or obese. In some embodiments, the patient has a body mass index (BMI) greater than or about 22 kg/m². In some embodiments, the patient has a BMI greater than or about 30 kg/m². In some embodiments, the methods for the treatment of type 2 diabetes mellitus comprise a reduction in fasting plasma glucose levels. In some embodiments, the fasting plasma glucose levels are reduced to about or below 100 mg/dL.

In some embodiments, the methods for the treatment of type 2 diabetes mellitus comprise a reduction in HbA1c levels. In some embodiments, the HbA1c levels are reduced to about or below 5.7%.

In some embodiments, the methods for the treatment of type 2 diabetes mellitus comprise a reduction in glucagon levels.

In some embodiments, the methods for the treatment of type 2 diabetes mellitus comprise an increase in insulin levels.

In some embodiments, the methods for the treatment of type 2 diabetes mellitus comprise a decrease in BMI. In some embodiments, the BMI is decreased to about or below 25 kg/m².

In some embodiments, the compound of Formula I, or a pharmaceutically acceptable salt or solvate thereof, or a pharmaceutical composition thereof, is administered orally.

In some embodiments, the methods of treatment for type 2 diabetes mellitus further comprise administering an additional therapy or therapeutic agent to the patient. In some embodiments, the additional therapy or therapeutic agent is selected from the group consisting of an antidiabetic agent, an anti-obesity agent, a GLP-1 receptor agonist, an agent to treat non-alcoholic steatohepatitis (NASH), an anti-emetic agent, gastric electrical stimulation, dietary monitoring, physical activity, or any combinations thereof. In some embodiments, the antidiabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof. In some embodiments, the biguanide is metformin. In some embodiments, the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, or any combinations thereof. In some embodiments, the GLP-1 receptor agonist is selected from the group consisting of liraglutide, exenatide, dulaglutide, albiglutide, taspoglutide, lixisenatide, semaglutide, or any combinations thereof. In some embodiments, the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASK1) inhibitor, or any combinations thereof. In some embodiments, the compound of Formula I, or a pharmaceutically acceptable salt or solvate thereof, or a pharmaceutical composition thereof, and the additional therapeutic agent are administered as separate dosages sequentially in any order.

Also provided herein are methods for modulating insulin levels in a patient in need of such modulating, the method comprising administering to the patient an effective amount of a compound of Formula I, or a pharmaceutically acceptable salt or solvate thereof, or a pharmaceutical composition thereof. In some embodiments, the modulation results in an increase of insulin levels.

Also provided herein are methods for modulating glucose levels in a patient in need of such modulating, the method comprising administering to the patient an effective amount of a compound of Formula I, or a pharmaceutically acceptable salt or solvate thereof, or a pharmaceutical composition thereof. In some embodiments, the modulation results in a decrease of glucose levels.

Also provided herein are methods for treating a GLP-1 associated disease, disorder, or condition, the method comprising administering to a patient in need thereof an effective amount of a compound of Formula I, or a pharmaceutically acceptable salt or solvate thereof, or a pharmaceutical composition thereof. In some embodiments, the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof. In some embodiments, the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof. In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

Other features and advantages of the invention will be apparent from the following detailed description and figures, and from the claims.

### DETAILED DESCRIPTION

Provided herein are heterocyclic GLP-1 agonists for use in the management of T2DM and other conditions where activation of GLP-1 activity is useful.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, the term “halo” or “halogen” means —F (sometimes referred to herein as “fluoro” or “fluoros”), —Cl (sometimes referred to herein as “chloro” or “chloros”), —Br (sometimes referred to herein as “bromo” or “bromos”), and —I (sometimes referred to herein as “iodo” or “iodos”).

As used herein, the term “alkyl” refers to saturated linear or branched-chain monovalent hydrocarbon radicals, containing the indicated number of carbon atoms. For example, “(C₁-C₆)alkyl” refers to saturated linear or branched-chain monovalent hydrocarbon radicals of one to six carbon atoms. Non-limiting examples of alkyl include methyl, ethyl, 1-propyl, isopropyl, 1-butyl, isobutyl, sec-butyl, tert-butyl, 2-methyl-2-propyl, pentyl, neopentyl, and hexyl.

As used herein, the term “alkylene” refers to a divalent alkyl containing the indicated number of carbon atoms. For example, “(C₁-C₃)alkylene” refers to a divalent alkyl having one to three carbon atoms (e.g., —CH₂—, —CH(CH₃)—, —CH₂CH₂—, or —CH₂CH₂CH₂—). Similarly, the terms “cycloalkylene”, “heterocycloalkylene”, “arylene”, and “heteroarylene” mean divalent cycloalkyl, heterocycloalkyl, aryl, and heteroaryl, respectively.

As used herein, the term “alkenyl” refers to a linear or branched mono-unsaturated hydrocarbon chain, containing the indicated number of carbon atoms. For example, “(C₂-C₆)alkenyl” refers a linear or branched mono unsaturated hydrocarbon chain of two to six carbon atoms. Non-limiting examples of alkenyl include ethenyl, propenyl, butenyl, or pentenyl.

As used herein, the term “alkynyl” refers to a linear or branched di-unsaturated hydrocarbon chain, containing the indicated number of carbon atoms. For example, “(C₂-C₆)alkynyl” refers to a linear or branched di-unsaturated hydrocarbon chain having two to six carbon atoms. Non-limiting examples of alkynyl include ethynyl, propynyl, butynyl, or pentynyl.

As used herein, the term “cycloalkyl” refers to a saturated or partially unsaturated cyclic hydrocarbon, containing the indicated number of carbon atoms. For example, “(C₃-C₆)cycloalkyl” refers to a saturated or partially unsaturated cyclic hydrocarbon having three to six ring carbon atoms. Non-limiting examples of cycloalkyl include cyclopropyl, cyclobutyl, cyclopentyl, and cyclohexyl. Cycloalkyl may be partially unsaturated. Non-limiting examples of partially unsaturated cycloalkyl include cyclohexenyl, cyclopentenyl, cycloheptenyl, cyclooctenyl, and the like. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

As used herein, the term “heterocycloalkyl” refers to a mon-, bi-, tri-, or polycyclic nonaromatic ring system containing indicated number of ring atoms (e.g., 3-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, the heteroatoms selected from O, N, S, or S(O)₁₋₂ (e.g., carbon atoms and 1-3, 1-6, or 1-9 heteroatoms of N, O, S, or S(O)₁₋₂ if monocyclic, bicyclic, or tricyclic, respectively), wherein 0, 1, 2 or 3 atoms of each ring may be substituted by a substituent. Examples of heterocycloalkyl groups include piperazinyl, pyrrolidinyl, dioxanyl, morpholinyl, tetrahydrofuranyl, and the like. Heterocycloalkyl groups may be partially unsaturated. Non-limiting examples of partially unsaturated heterocycloalkyl include dihydropyrrolyl, dihydropyridinyl, tetrahydropyridinyl, dihydrofuranyl, dihydropyranyl, and the like. Heterocycloalkyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocycloalkyl also includes spirocyclic rings (e.g., spirocyclic bicycle wherein two rings are connected through just one atom). Non-limiting examples of spirocyclic heterocycloalkyl include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, the term “aryl” refers to a mono-, bi-, tri- or polycyclic hydrocarbon group containing the indicated numbers of carbon atoms, wherein at least one ring in the system is aromatic (e.g., C₆ monocyclic, C₁₀ bicyclic, or C₁₄ tricyclic aromatic ring system). Examples of aryl groups include phenyl, naphthyl, tetrahydronaphthyl, and the like.

As used herein, the term “heteroaryl” refers to a mono-, bi-, tri- or polycyclic group having indicated numbers of ring atoms (e.g., 5-6 ring atoms; e.g., 5, 6, 9, 10, or 14 ring atoms); wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others.

As used herein, the term “haloalkyl” refers to an alkyl radical as defined herein, wherein one or more hydrogen atoms is replaced with one or more halogen atoms. Non-limiting examples include fluoromethyl, difluoromethyl, trifluoromethyl, 2-fluoroethyl, 2,2-difluoroethyl, 2,2,2-trifluoroethyl, chloromethyl, dichloromethyl, chloroethyl, trichloroethyl, bromomethyl, and iodomethyl.

As used herein, the term “alkoxy” refers to an —O-alkyl radical, wherein the radical is on the oxygen atom. For example, “C₁₋₆ alkoxy” refers to an —O—(C₁₋₆ alkyl) radical, wherein the radical is on the oxygen atom. Examples of alkoxy include methoxy, ethoxy, propoxy, isopropoxy, butoxy and tert-butoxy. Accordingly, as used herein, the term “haloalkoxy” refers to an —O-haloalkyl radical, wherein the radical is on the oxygen atom.

As used herein, “ [symbol] ” indicates an optional single or double bond, as allowed by valence. As used herein, “ [symbol] ” indicates the point of attachment to the parent molecule.

As used herein, the term “compound,” is meant to include all stereoisomers, geometric isomers, tautomers, and isotopes of the structures depicted. Compounds herein identified by name or structure as one particular tautomeric form are intended to include other tautomeric forms unless otherwise specified.

As used herein, when a ring is described as being “aromatic”, it means the ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thioazole, isoxazole, isothiazole, and the like. When a ring system comprising at least two rings is described as “aromatic”, it means the ring system comprises one or more aromatic ring(s). Accordingly, when a ring system comprising at least two rings is described as “non-aromatic”, none of the constituent rings of the ring system is aromatic.

As used herein, when a ring is described as being “partially unsaturated”, it means the ring has one or more additional degrees of unsaturation (in addition to the degree of unsaturation attributed to the ring itself, e.g., one or more double bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like. When a ring system comprising at least two rings is described as “partially unsaturated”, it means the ring system comprises one or more partially unsaturated ring(s), provided that none of the constituent rings of the ring system is aromatic.

As used herein, the term “carboxylic acid bioisostere” means a group which has chemical and physical similarities producing broadly similar biological properties to a carboxylic acid (see Lipinski, Annual Reports in Medicinal Chemistry, 1986, 21, p 283 “Bioisosterism In Drug Design”; Yun, Hwahak Sekye, 1993, 33, pages 576-579 “Application Of Bioisosterism To New Drug Design”; Zhao, Huaxue Tongbao, 1995, pages 34-38 25 “Bioisosteric Replacement And Development Of Lead Compounds In Drug Design”; Graham, Theochem, 1995, 343, pages 105-109 “Theoretical Studies Applied To Drug Design:ab initio Electronic Distributions In Bioisosteres”). Examples of suitable carboxylic acid bioisostere include: sulfo, phosphono, alkylsulfonylcarbamoyl, tetrazolyl, arylsulfonylcarbamoyl, heteroarylsulfonylcarbamoyl, N-methoxycarbamoyl, 3-hydroxy-3-cyclobutene-1,2-dione, 3,5-dioxo-1,2,4-oxadiazolidinyl or heterocyclic phenols such as 3-hydroxyisoxazolyl and 3-hydoxy-1-methylpyrazolyl.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the invention, and the naming of the compounds does not exclude any tautomer.

The term “GLP-1R” or “GLP-1 receptor” as used herein is meant to include, without limitation, nucleic acids, polynucleotides, oligonucleotides, sense and antisense polynucleotide strands, complementary sequences, peptides, polypeptides, proteins, homologous, and/or orthologous GLP-1R molecules, isoforms, precursors, mutants, variants, derivatives, splice variants, alleles, different species, and active fragments thereof.

The term “GLP-1 associated disease” as used herein is meant to include, without limitation, all those diseases, disorders, or conditions in which modulating glucagon-like peptide-1 (GLP-1) receptor signaling can alter the pathology and/or symptoms and/or progression of the disease, disorder, or condition.

The term “GLP-1 agonist” or “GLP-1 RA” as used herein refers to an agonist of the glucagon-like peptide-1 (GLP-1) receptor. GLP-1 RAs enhance glucose-dependent insulin secretion; suppress inappropriately elevated glucagon levels, both in fasting and postprandial states; and slow gastric emptying. Karla et al., Glucagon-like peptide-1 receptor agonists in the treatment of type 2 diabetes: Past, present, and future, Indian J Endocrinol Metab. 2016 March-April; 20(2): 254-267. GLP-1 RAs have been shown to treat type 2 diabetes. Examples of GLP-1 RAs include, but are not limited to, albiglutide (TANZEUM®), dulaglutide (LY2189265, TRULICITY®), efpeglenatide, exenatide (BYETTA®, BYDUREON®, Exendin-4), liraglutide (VICTOZA®, NN2211), lixisenatide (LYXUMIA®), semaglutide (OZEMPIC®), tirzepatide, ZP2929, NNC0113-0987, BPI-3016, and TT401. See, also, for example, additional GLP-1 recceptor agonists described in U.S. Pat. Nos. 10,370,426; 10,308,700; 10,259,823; 10,208,019; 9,920,106; 9,839,664; 8,129,343; 8,536,122; 7,919,598; 6,414,126; 6,628,343; and RE45313.

The term “pharmaceutically acceptable” as used herein indicates that the compound, or salt or composition thereof is compatible chemically and/or toxicologically with the other ingredients comprising a formulation and/or the patient being treated therewith.

The term “therapeutic compound” as used herein is meant to include, without limitation, all compounds of Formula I, or pharmaceutically acceptable salts or solvates thereof (e.g., a compound of any one of Formulas (I-A1), (I-A3), (I-A4), and (I-A5), or a pharmaceutically acceptable salt or solvate thereof), and all compositions (e.g., pharmaceutical compositions) wherein a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) is a component of the composition.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof)) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutical composition” refers to a mixture of a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) as described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing”, as used herein, is the prevention of the onset, recurrence or spread, in whole or in part, of the disease or condition as described herein, or a symptom thereof.

The terms “subject”, “patient” or “individual”, as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the patient is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent in a combination of the invention.

The term “pharmaceutical combination”, as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulation”, as used herein, refers to a regulation or an adjustment (e.g., increase or decrease) and can include, for example agonism, partial agonism or antagonism.

### Compounds

Accordingly, provided herein are compounds of Formula I:

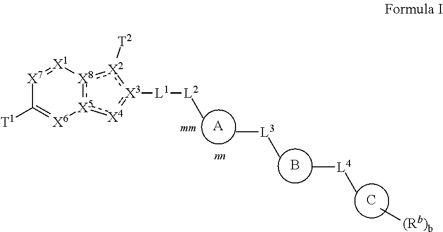

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

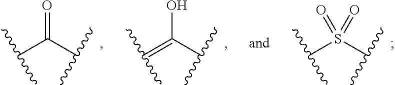

5- to 10-membered heterocycloalkyl, wherein the heterocycloalkyl comprises an endocyclic group selected from the group consisting of:

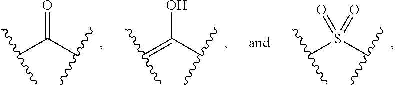

wherein the heterocycloalkyl is optionally substituted with 1-4 R^{v}; and
(C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 6-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T};
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

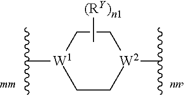

wherein n1 is 0, 1, or 2; W¹ is CR^{Y1} or N; and W² is CR^{Y2} or N;

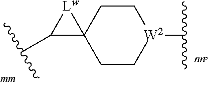

wherein W² is CR^{Y2} or N, L^{w} is (C₁-C₃)alkylene;
phenylene optionally substituted with 1-4 R^{Y};
5- to 6-membered heteroarylene optionally substituted with 1-3 R^{Y};
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y};
wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is a bond;
Ring B is selected from the group consisting of: (B-I), (B-II), (B-III), (B-IV), and (B-V):

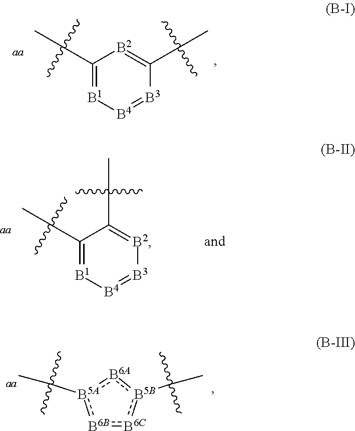

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

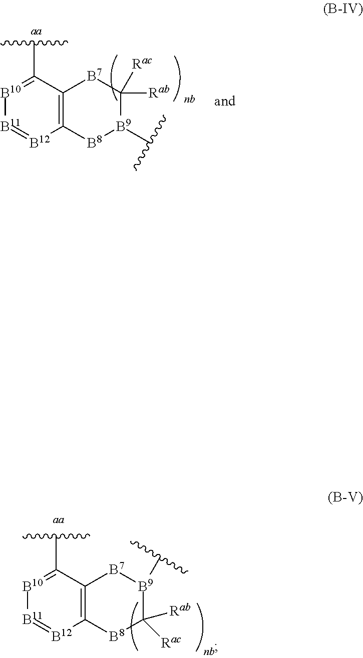

wherein aa represents the point of attachment to L³;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

Also provided herein are compounds of Formula I:

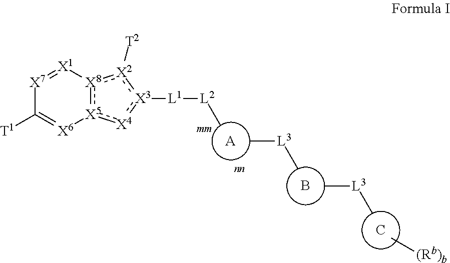

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

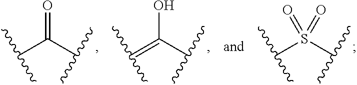

5- to 10-membered heterocycloalkyl, wherein the heterocycloalkyl comprises an endocyclic group selected from the group consisting of:

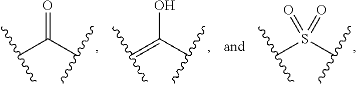

wherein the heterocycloalkyl is optionally substituted with 1-4 R^{v}; and
(C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring; or when q is 2 or 3, a pair of R^{x} on adjacent carbon atoms, taken together form a double bond between the adjacent carbon atoms;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 10-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, (C₃-C₈)cycloalkyl, —O(C₁-C₆)alkylene-O(C₁-C₆)alkyl, —OH, —N(R^{s})₂ 3- to 8-membered heterocycloalkyl, —CO₂H, (C₁-C₆)hydroxyalkyl, (C₁-C₆)alkyl-S(O)₁₋₂—(C₁-C₆)alkyl, (C₁-C₆)alkyl-C(═O)OH, —S(O)₀₋₂—C₁-C₆ alkyl, (C₁-C₆)alkenyl, —P(═O)R^{z1}R^{z2}, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T};
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, —C(═O)C₁-C₆ alkyl, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

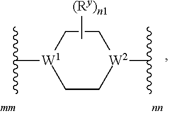

wherein n1 is 0, 1, or 2; W¹ is CR^{Y1} or N; and W² is CR^{Y2} or N;

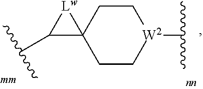

wherein W² is CR^{Y2} or N, L^{w} is (C₁-C₃)alkylene;
   phenylene optionally substituted with 1-4 R^{Y};
   5- to 6-membered heteroarylene optionally substituted with 1-3 R^{Y};
   partially unsaturated monocyclic (C₅-C₈)cycloalkylene optionally substituted with 1-4 R^{Y}; and
   partially unsaturated monocyclic 5- to 8-membered heterocycloalkylene optionally substituted with 1-4 R^{Y};
wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is a bond;
Ring B is selected from the group consisting of: (B-I), (B-II), (B-III), (B-IV), and (B-V):

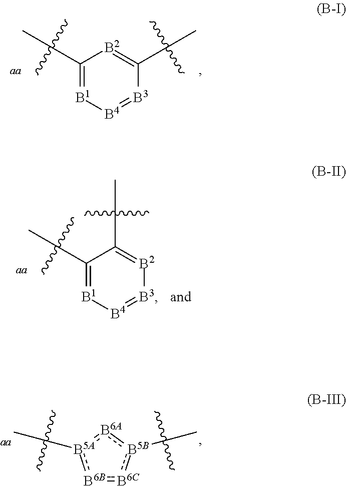

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B⁶C is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

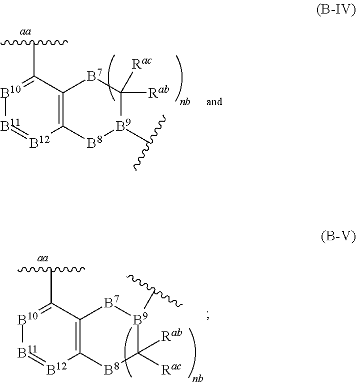

wherein aa represents the point of attachment to L³;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

Further provided herein are compounds of A Formula I:

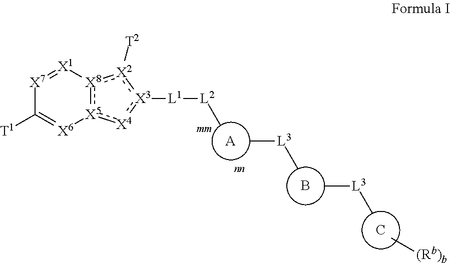

or a pharmaceutically acceptable salt or solvate thereof, wherein:
[symbol] indicates an optional single or double bond, as allowed by valence;
each of X¹, X², X⁴, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

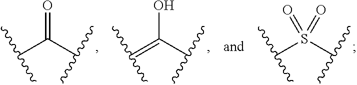

5- to 10-membered heterocycloalkyl, wherein the heterocycloalkyl comprises an endocyclic group selected from the group consisting of:

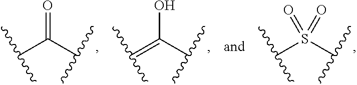

wherein the heterocycloalkyl is optionally substituted with 1-4 R^{v}; and
(C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring; or when q is 2 or 3, a pair of R^{x} on adjacent carbon atoms, taken together form a double bond between the adjacent carbon atoms;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 10-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, (C₃-C₈)cycloalkyl, —O(C₁-C₆)alkylene-O(C₁-C₆)alkyl, —OH, —N(R^{s})₂ 3- to 8-membered heterocycloalkyl, —CO₂H, (C₁-C₆)hydroxyalkyl, (C₁-C₆)alkyl-S(O)₁₋₂—(C₁-C₆)alkyl, (C₁-C₆)alkyl-C(═O)OH, —S(O)₀₋₂—C₁-C₆ alkyl, (C₁-C₆)alkenyl, —P(═O)R^{z1}R^{z2}, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T};
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, —C(═O)C₁-C₆ alkyl, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

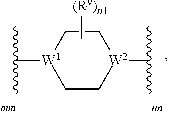

wherein n1 is 0, 1, or 2; W¹ is CR^{Y1} or N; and W² is CR^{Y2} or N;

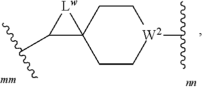

wherein W² is CR^{Y2} or N, L^{w} is (C₁-C₃)alkylene;
   phenylene optionally substituted with 1-4 R^{Y};
   5- to 6-membered heteroarylene optionally substituted with 1-3 R^{Y};
   partially unsaturated monocyclic (C₅-C₈)cycloalkylene optionally substituted with 1-4 R^{Y}; and
   partially unsaturated monocyclic 5- to 8-membered heterocycloalkylene optionally substituted with 1-4 R^{Y};
wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is a bond;
Ring B is selected from the group consisting of: (B-I), (B-II), (B-III), (B-IV), and (B-V):

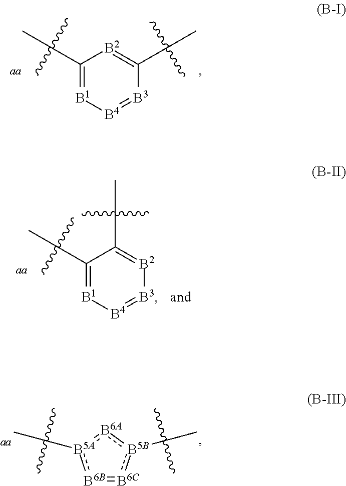

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B⁶C is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

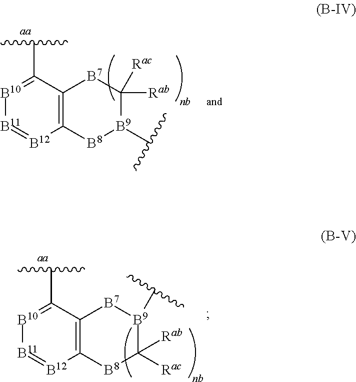

wherein aa represents the point of attachment to L³;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, CN, —C(O)NH₂, —CONH(C₁-C₃ alkyl) and C(O)N(C₁-C₃)alky)₂; and
b is an integer selected from 0-3.

In some embodiments of Formula I, T¹ is -T³. In some embodiments of Formula I, T¹ is -L^{a}-(CR^{x}R^{x})_{q}-T³.

In some embodiments of Formula I, -L^{a} is a bond. In some embodiments of Formula I, -L^{a} is —NH—, —N(C1-3 alkyl)-, —O—, or —S—. As a non-limiting example of the foregoing embodiments, -L^{a} can be —O—.

In some embodiments of Formula I, q is 1. In some embodiments of Formula I, q is 2 or 3.

In some embodiments of Formula I, each R^{x} is hydrogen or (C₁-C₆)alkyl. As a non-limiting example of the foregoing embodiments, each R^{x} can be hydrogen. In some embodiments, one pair of R^{x} on the same carbon, taken together with the carbon atom to which each is attached, forms a (C₃-C₈)cycloalkyl ring. As a non-limiting example of the foregoing embodiments, one pair of R^{x} on the same carbon, taken together with the carbon atom to which each is attached, can form a cyclopropyl. In some embodiments, each remaining R^{x} is hydrogen.

In some embodiments of Formula I, T¹ is —(CR^{x}R^{x})-T³. In some embodiments, each R^{x} is hydrogen. In some embodiments, the pair of R^{x} taken together with the carbon atom to which each is attached, forms a (C₃-C₈)cycloalkyl ring. For example, a pair of R^{x} taken together with the carbon atom to which each is attached, can form a cyclopropyl ring.

In some embodiments of Formula I, T¹ is —(CR^{x}R^{x})_{q}-T³; and q is 2 or 3. In some embodiments, each R^{x} is hydrogen.

In some embodiments of Formula I, T¹ is —O—(CR^{x}R^{x})_{q}-T³; and q is 1, 2, or 3.

In some embodiments, q is 1. In some embodiments, q is 2 or 3. In some embodiments, each R^{x} is hydrogen. In some embodiments, q is 1; and each R^{x} is hydrogen.

In some embodiments of Formula I, T³ is selected from the group consisting of: —N(R^{s})C(═O)R^{z}, —N(R^{s})C(═O)OR^{z}, —N(R^{s})C(═O)N(R^{s})R^{z}, —N(R^{s})S(O)₁₋₂—R^{z}, and —N(R^{s})S(═NR^{s})(═O)R^{z}.

In some embodiments of Formula I, T³ is —N(R^{s})C(═O)R^{z}. In some embodiments, T³ is —NHC(═O)R^{z}.

In some embodiments of Formula I, T³ is —N(R^{s})S(O)₁₋₂—R^{z} (e.g., —N(R^{s})S(O)₂—R^{z}). In some embodiments, T³ is —NHS(O)₂R^{z}.

In some embodiments of Formula I, T³ is —N(R^{s})C(═O)N(R^{s})R^{z}. In some embodiments, T³ is —NHC(═O)NHR^{z}.

In some embodiments of Formula I, T³ is —N(R^{s})C(═O)OR^{z}. In some embodiments, T³ is —NHC(═O)OR^{z}.

In some embodiments of Formula I, T³ is —S(O)₁₋₂R^{z}. In some embodiments, T³ is —S(O)₂R^{z}.

In some embodiments of Formula I, T³ is selected from the group consisting of: —C(═O)N(R^{s})R^{z}, —S(O)₁₋₂N(R^{s})R^{z}, and —S(═NR^{s})(═O)N(R^{s})R^{z}.

In some embodiments of Formula I, T³ is —C(═O)N(R^{s})R^{z}. In some embodiments, T³ is —C(═O)NR^{z}.

In some embodiments of Formula I, T³ is —S(O)₂N(R^{s})R^{z}. In some embodiments, T³ is —S(O)₂NR^{z}.

In some embodiments of Formula I, T³ is —S(═NR^{s})(═O)N(R^{s})R^{z}. In some embodiments, T³ is —S(═NH)(═O)N(R^{s})R^{z}. For example, T³ can be —S(═NH)(═O)N(H)R^{z}.

In some embodiments of Formula I, R^{z} is hydrogen.

In some embodiments of Formula I, R^{z} is (C₁-C₆)alkyl optionally substituted with 1-4 R^{v}.

In some embodiments of Formula I, R^{z} is (C₁-C₃)alkyl. As non-limiting examples of the foregoing embodiments, R^{z} can be methyl, ethyl, or isopropyl.

In some embodiments of Formula I, R^{z} is (C₁-C₃)alkyl substituted with 1-3 R^{v}. In some embodiments, R^{z} is (C₁-C₃)alkyl substituted with from 1-3 substituents each independently selected from the group consisting of halo and (C₁-C₃)alkoxy. For example, R^{z} can be (C₁-C₃)alkyl substituted with 1-3 independently selected halo, such as —CH₂CF₃. As another non-limiting example, R^{z} can be (C₁-C₃)alkyl substituted with (C₁-C₃)alkoxy, such as —CH₂CH₂OMe.

In some embodiments of Formula I, R^{z} is —R^{z3} or -L^{b}-R^{z3}. In some embodiments, R^{z} is —R^{z3} or —CH₂—R^{z3}. In some embodiments, R^{z3} is selected from the group consisting of: (C₃-C₆)cycloalkyl and 3- to 6-membered heterocycloalkyl, each of which is optionally substituted with 1-4 R^{v}. In some embodiments, R^{z3} is selected from the group consisting of: (C₃-C₆)cycloalkyl and 3- to 6-membered heterocycloalkyl, each optionally substituted with 1-2 R^{v}. As non-limiting examples of the foregoing embodiments, R^{z3} can be cyclopropyl, cyclobutyl, difluorocyclobutyl

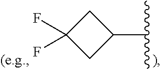 [SMILES: *C1CC(F)(F)C1]

cyclopentyl, oxetanyl

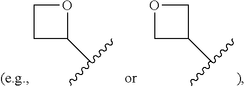 [SMILES: *C1CCO1 ; *C1COC1]

tetrahydropyranyl

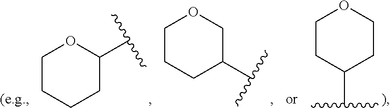

or tetrahydro-2H-thiopyran 1,1-dioxide

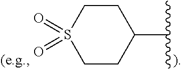 [SMILES: *C1CCS(=O)(=O)CC1]

Other non-limiting examples include 1-4, dioxanyl and piperidinyl. Still other non-limiting examples include:

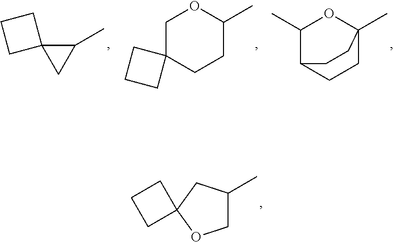

and optionally substituted cyclopentyl.

In some embodiments of Formula I, R^{z3} is selected from the group consisting of: (C₆-C₁₀)aryl and 5- to 10-membered heteroaryl, each of which is optionally substituted with 1-4 R^{v}. In some embodiments, R^{z3} is phenyl optionally substituted with 1-2 R^{v}. In certain embodiments, R is cyano or haloalkyl, such as CF₃. For example, R^{z3} can be unsubstituted phenyl. In some embodiments, R^{z3} is 5- to 10-membered heteroaryl (e.g., 5- to 6-membered heteroaryl) optionally substituted with 1-2 R^{v}. For example, R^{z3} can be 2-pyridyl, 3-pyridyl, or 4-pyridyl optionally substituted with 1-2 R^{v} (e.g., (C₁-C₆)alkyl, such as methyl or ethyl; (C₁-C₆)alkoxy, such as methoxy; (C₃-C₆)cycloalkyl, such as cyclopropyl; —O—CH₂CH₂—OCH₃; halo (e.g., F); and haloalkyl (e.g., CF₃). An another example, R^{z3} can be pyrazolyl.

In some embodiments of Formula I, T³ is —NHC(═O)R^{z} or —NHS(O)₁₋₂—R^{z}; and R^{z} is (C₁-C₆)alkyl optionally substituted with 1-4 R^{v}. In some embodiments, R^{z} is (C₁-C₃)alkyl (e.g., methyl, ethyl, or isopropyl). In some embodiments, R^{z} is (C₁-C₃)alkyl substituted with (C₁-C₃)alkoxy (e.g., —CH₂CH₂OMe). In some embodiments, R^{z} is (C₁-C₃)alkyl substituted with 1-3 halo (e.g., —CH₂CF₃). In some embodiments, T³ is —NHC(═O)R^{z}. In some embodiments, T³ is —NHS(O)₂—R^{z}.

In some embodiments of Formula I, T³ is —NHC(═O)R^{z} or —NHS(O)₁₋₂—R^{z}; and R^{z} is —R^{z3} or —CH₂—R^{z3}. In some embodiments, R^{z3} is selected from the group consisting of: phenyl, (C₃-C₆)cycloalkyl, and 3- to 6-membered heterocycloalkyl, each optionally substituted with 1-2 R^{v}. As non-limiting examples, R^{z3} can be phenyl, cyclopropyl, cyclobutyl, difluorocyclobutyl

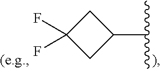 [SMILES: *C1CC(F)(F)C1]

cyclopentyl, oxetanyl

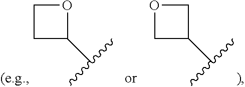 [SMILES: *C1CCO1 ; *C1COC1]

tetrahydropyranyl

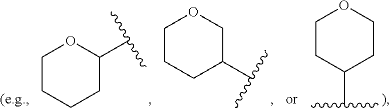

or tetrahydro-2H-thiopyran 1,1-dioxide

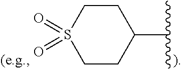 [SMILES: *C1CCS(=O)(=O)CC1]

Other non-limiting examples include 1-4, dioxanyl and piperidinyl. Still other non-limiting examples include:

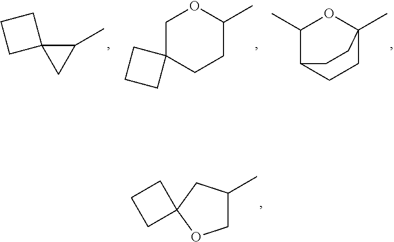

and optionally substituted cyclopentyl. In some embodiments, R^{z3} is 5- to 10-membered heteroaryl (e.g., 5- to 6-membered heteroaryl) optionally substituted with 1-2 R^{v}. For example, R^{z3} can be 2-pyridyl, 3-pyridyl, or 4-pyridyl optionally substituted with 1-2 R^{v} (e.g., (C₁-C₆)alkyl, such as methyl or ethyl; (C₁-C₆)alkoxy, such as methoxy; (C₃-C₆)cycloalkyl, such as cyclopropyl; —O—CH₂CH₂—OCH₃; halo (e.g., F); and haloalkyl (e.g., CF₃). An another example, R^{z3} can be pyrazolyl. In some embodiments, T³ is —NHC(═O)R^{z}. In some embodiments, T³ is —NHS(O)₂—R^{z}.

In some embodiments of Formula I, T³ is —S(O)₂R^{z}; and R^{z} is (C₁-C₆)alkyl optionally substituted with 1-4 R^{v}. In some embodiments, R^{z} is (C₁-C₆)alkyl. For example, R^{z} can be (C₁-C₃)alkyl

In some embodiments of Formula I, T³ is selected from the group consisting of: —C(═O)N(R^{s})R^{z}, —S(O)₁₋₂N(R^{s})R^{z}, and —S(═NR^{s})(═O)N(R^{s})R^{z}; and R^{z} is hydrogen or (C₁-C₆)alkyl optionally substituted with 1-4 R^{v}. In some embodiments, T³ is —C(═O)N(R^{s})R^{z}. For example, T³ can be —C(═O)NR^{z}. In some embodiments, T³ is —S(O)₂N(R^{s})R^{z}. For example, T³ can be —S(O)₂NHR^{z}. In some embodiments, T³ is —S(═NR^{s})(═O)N(R^{s})R^{z}. For example, T³ can be —S(═NH)(═O)N(R^{s})R^{z} (e.g., —S(═NH)(═O)N(H)R^{z}). In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula I, T³ is —N(R^{s})C(═O)N(R^{s})R^{z} or —N(R^{s})C(═O)OR^{z}; and R^{z} is hydrogen or (C₁-C₆)alkyl optionally substituted with 1-4 R^{v}. In some embodiments, T³ is —NHC(═O)NR^{z}. In some embodiments, T³ is —N(H)C(═O)OR^{z}. In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula I, T³ is —C(═O)OH.

In some embodiments of Formula I, T³ is —P(═O)R^{z1}R^{z2}. In some embodiments, R^{z1} and R^{z2} are each independently selected (C₁-C₃)alkyl. For example, each R^{z1} can be independently methyl or ethyl.

In some embodiments of Formula I, T³ is 5- to 10-membered heteroaryl optionally substituted with 1-4 R^{v}, and wherein the heteroaryl optionally comprises an endocyclic group selected from the group consisting of:

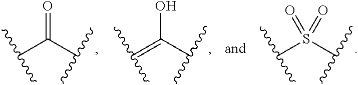

In some embodiments of Formula I, T³ is 5- to 6-membered heteroaryl optionally substituted with 1-4 R^{v}. In some embodiments, T³ is 5-membered heteroaryl having 2-4 ring heteroatoms selected from the group consisting of N, O, and S, wherein the heteroaryl is optionally substituted with 1-2 R^{v}. As non-limiting examples of the foregoing embodiments, T³ can be selected from the group consisting of:

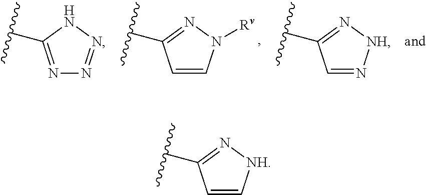

Other non-limiting examples include 1,2,4-triazolyl, e.g.,

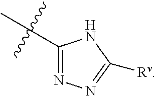

In certain embodiments, R^{v} is (C₁-C₃)haloalkyl (e.g., CF₃); oxetanyl, —P(O)Me₂, (C₁-C₃) alkyl (e.g., methyl); or (C₃-C₆)cyclooalkyl (e.g., cyclopropyl). Further non-limiting examples include optionally substituted pyridyl and bicyclic heteroaryl rings, e.g.,

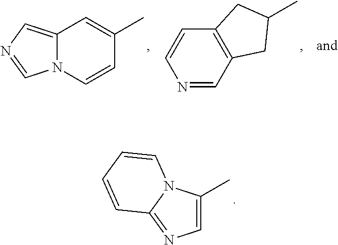

In some embodiments of Formula I, T³ is 5- to 6-membered heteroaryl that comprises an endocyclic group selected from the group consisting of:

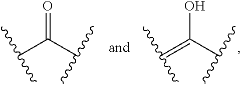

wherein the heteroaryl is further optionally substituted with 1-4 R^{v}. As non-limiting examples of the foregoing embodiments, T³ can be selected from the group consisting of:

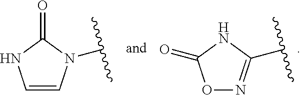 [SMILES: *n1cc[nH]c1=O ; *c1noc(=O)[nH]1]

Other non-limiting examples include:

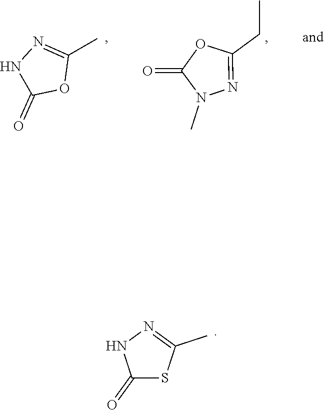

In some embodiments of Formula I, T³ is 5- to 10-membered heterocycloalkyl, wherein the heterocycloalkyl comprises an endocyclic group selected from the group consisting of:

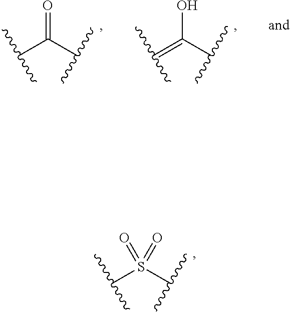

wherein the heterocycloalkyl is optionally substituted with 1-4 R^{v}. In some embodiments, T³ is 5- to 6-membered heterocycloalkyl which comprises an endocyclic group selected from the group consisting of:

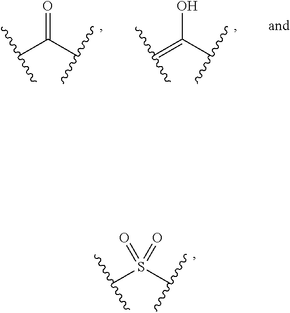

wherein the heterocycloalkyl is optionally substituted with 1-4 R^{v}.

In some embodiments, T³ is

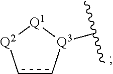

Q¹ is C(═O) or S(O)₂; Q² is O, NH, —CH₂—, or —CH₂—CH₂—; Q³ is N or CH; and [symbol] is a single bond or a double bond, provided that T³ is non-aromatic. As non-limiting examples of the foregoing embodiments, T³ can be selected from the group consisting of:

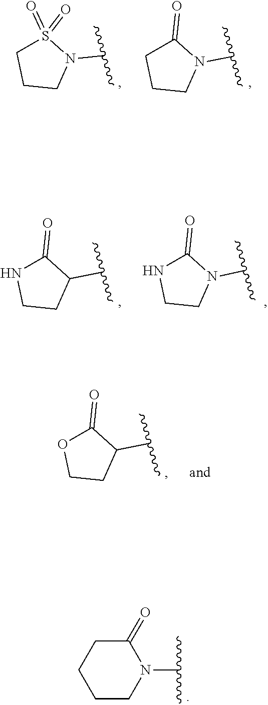

Another non-limiting example is

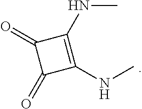

In some embodiments of Formula I, T³ is (C₁-C₆)haloalkyl substituted with —OH and further optionally substituted with 1-2 R^{v}. In some embodiments, T³ is (C₁-C₆)haloalkyl substituted with —OH and further optionally substituted with 1-2 R^{v}. In some embodiments, T³ is (C₁-C₆)haloalkyl substituted with —OH. As a non-limiting example of the foregoing embodiments, T¹ can be

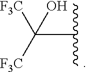 [SMILES: *C(O)(C(F)(F)F)C(F)(F)F]

In some embodiments of Formula I, T¹ is —N(R^{s})C(═O)R^{z} or —N(R^{s})S(O)₁₋₂—R^{z}; and R^{z} is (C₁-C₆)alkyl optionally substituted with 1-4 R^{v}. In some embodiments, R^{z} is (C₁-C₃)alkyl (e.g., methyl, ethyl, or isopropyl). In some embodiments, R^{z} is (C₁-C₃)alkyl substituted with from 1-3 substituents each independently selected from the group consisting of halo and (C₁-C₃)alkoxy. For example, R^{z} can be —CH₂CF₃ or —CH₂CH₂OMe. In some embodiments, T¹ is —NHC(═O)R^{z}. In some embodiments, T¹ is —NHS(O)₂—R^{z}.

In some embodiments of Formula I, T¹ is —N(R^{s})C(═O)R^{z} or —N(R^{s})S(O)₁₋₂—R^{z}; and R^{z} is —R^{z3} or —CH₂—R^{z3}. In some embodiments, R^{z3} is selected from the group consisting of: phenyl, (C₃-C₆)cycloalkyl, and 3- to 6-membered heterocycloalkyl, each optionally substituted with 1-2 R^{v}. As non-limiting examples of the foregoing embodiments, R^{z3} can be phenyl, cyclopropyl, cyclobutyl, difluorocyclobutyl

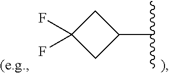 [SMILES: *C1CC(F)(F)C1]

cyclopentyl, oxetanyl

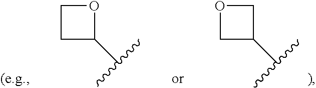 [SMILES: *C1CCO1 ; *C1COC1]

tetrahydropyranyl

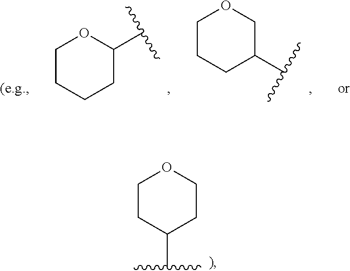 [SMILES: *C1CCCCO1 ; *C1CCCOC1 ; *C1CCOCC1]

or tetrahydro-2H-thiopyran 1,1-dioxide

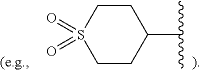 [SMILES: *C1CCS(=O)(=O)CC1]

In some embodiments, T¹ is —NHC(═O)R^{z}. In some embodiments, T¹ is —NHS(O)₂—R^{z}.

In some embodiments of Formula I, T¹ is —(CR^{x}R^{x})_{q}—S(O)₂R^{z}; and q is 1, 2, or 3. In some embodiments, R^{z} is (C₁-C₆)alkyl optionally substituted with 1-4 R^{v}. In some embodiments, R^{z} is (C₁-C₃)alkyl. In some embodiments, q is 1. In some embodiments, q is 2. In some embodiments, each R^{x} is hydrogen.

In some embodiments of Formula I, T¹ is selected from the group consisting of: —C(═O)N(R^{s})R^{z}, —S(o)₁₋₂N(R^{s})R^{z}, and —S(═NR^{s})(═O)N(R^{s})R^{z}. In some embodiments, T¹ is —C(═O)N(R^{s})R^{z}. For example, T¹ can be —C(═O)NR^{z}. In some embodiments, T¹ is —S(O)₂N(R^{s})R^{z}. For example, T¹ can be —S(O)₂NR^{z}. In some embodiments, T¹ is —S(═NR^{s})(═O)N(R^{s})R^{z}. For example, T can be —S(═NH)(═O)N(R^{s})R^{z} (e.g., —S(═NH)(═O)N(H)R^{z}). In some embodiments, R^{z} is hydrogen or (C₁-C₆)alkyl optionally substituted with 1-4 R^{v}. In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula I, T is —N(R^{s})C(═O)N(R^{s})R^{z} or —N(R^{s})C(═O)OR^{z}. In some embodiments, T¹ is —NHC(═O)NHR^{z}. In some embodiments, T¹ is —N(H)C(═O)OR^{z}. In some embodiments, R^{z} is hydrogen or (C₁-C₆)alkyl optionally substituted with 1-4 R^{v} In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula I, T¹ is —(CR^{x}R^{x})_{q}—C(═O)OH; and q is 1, 2, or 3. In some embodiments, q is 1. In some embodiments, q is 2. In some embodiments, each R^{x} is hydrogen. In some embodiments (e.g., when q is 1), one pair of R^{x} on the same carbon, taken together with the carbon atom to which each is attached, forms a (C₃-C₈)cycloalkyl ring; and each remaining R^{x} when present is hydrogen. As a non-limiting example of the foregoing embodiments, the pair R^{x} on the same carbon, taken together with the carbon atom to which each is attached, can form a cyclopropyl ring. As non-limiting examples of the foregoing embodiments, T¹ can be

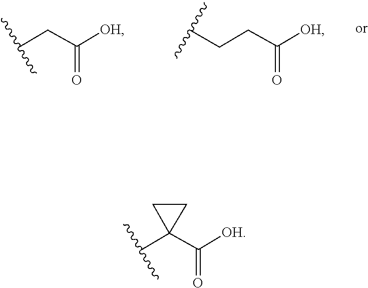 [SMILES: *CC(=O)O ; *CCC(=O)O ; "or" ; *C1(C(=O)O)CC1]

In some embodiments of Formula I, T¹ is —C(═O)OH.

In some embodiments of Formula I, T¹ is —O—(CR^{x}R^{x})_{q}—C(═O)OH; and q is 1, 2, or 3. In some embodiments, q is 1. In some embodiments, each R^{x} is hydrogen.

In some embodiments of Formula I, T¹ is —P(═O)R^{z1}R^{z2}. In some embodiments, R^{z1} and R^{z2} are independently selected (C₁-C₃)alkyl. For example, R^{z1} and R^{z2} can be independently methyl or ethyl.

In some embodiments of Formula I, T¹ is 5-membered heteroaryl having 2-4 ring heteroatoms selected from the group consisting of N, O, and S, wherein the heteroaryl is optionally substituted with 1-2 R^{v}. As non-limiting examples of the foregoing embodiments, T¹ can be selected from the group consisting of:

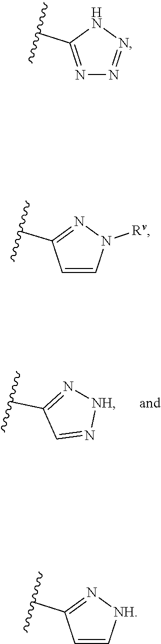

In some embodiments of Formula I, T¹ is 5- to 6-membered heteroaryl that comprises an endocyclic group selected from the group consisting of:

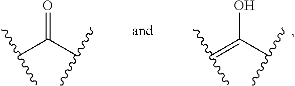

wherein the heteroaryl is further optionally substituted with 1-4 R^{v}. As non-limiting examples of the foregoing embodiments, T¹ can be selected from the group consisting of:

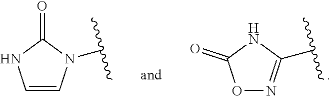 [SMILES: *n1cc[nH]c1=O ; *c1noc(=O)[nH]1]

In some embodiments, T¹ is

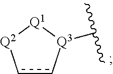

Q¹ is C(═O) or S(O)₂; Q² is O, NH, —CH₂—, or —CH₂—CH₂—; Q³ is N or CH; and [symbol] is a single bond or a double bond, provided that T¹ is non-aromatic. As non-limiting examples of the foregoing embodiments, T¹ can be selected from the group consisting of:

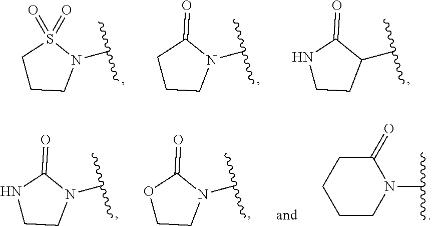 [SMILES: *N1CCCS1(=O)=O ; *N1CCCC1=O ; *C1CCNC1=O ; *N1CCNC1=O ; *N1CCOC1=O ; *N1CCCCC1=O]

In some embodiments of Formula I, T¹ is (C₁-C₆)haloalkyl substituted with —OH and further optionally substituted with 1-2 R^{v}. In some embodiments, T¹ is (C₁-C₆)haloalkyl substituted with —OH. As a non-limiting example of the foregoing embodiments, T can be

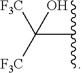 [SMILES: *C(O)(C(F)(F)F)C(F)(F)F]

In some embodiments of Formula I, X² is N; and X⁴ is N.

In some embodiments of Formula I, X⁸ is C; and X⁵ is C.

In some embodiments of Formula I, X³ is C.

In some embodiments of Formula I, X² is N; X³ is C; X⁴ is N; X⁵ is C; and X⁸ is C.

In some embodiments of Formula I, each of X¹, X⁷, and X⁶ is independently CH or CR^{w}. In some embodiments, X² is N; X³ is C; X⁴ is N; X⁵ is C; and X⁸ is C.

In some embodiments of Formula I, each of X¹, X⁷, and X⁶ is CH. In some embodiments, X² is N; X³ is C; X⁴ is N; X⁵ is C; and X⁸ is C.

In some embodiments of Formula I, one of X¹, X⁷, and X⁶ is CR^{w}; and each remaining of X¹, X⁷, and X⁶ is CH. In some embodiments, X² is N; X³ is C; X⁴ is N; X⁵ is C; and X⁸ is C.

In some embodiments of Formula I, X⁶ is CR^{w}; and X¹ and X⁷ are CH. As a non-limiting example of the foregoing embodiments, X⁶ can be C—F. In some embodiments, X² is N; X³ is C; X⁴ is N; X⁵ is C; and X⁸ is C.

In some embodiments of Formula I, one of X¹, X⁷, and X⁶ is N; and each remaining of X¹, X⁷, and X⁶ is CH or CR^{w}. In some embodiments of Formula I, X¹ is N; and X⁶ and X⁷ are CH. In some embodiments of Formula I, X⁶ is N; and X¹ and X⁷ are CH. In some embodiments of Formula I, X⁷ is N; and X¹ and X⁶ are CH. In some embodiments, X² is N; X³ is C; X⁴ is N; X⁵ is C; and X⁸ is C.

In some embodiments of Formula I, the

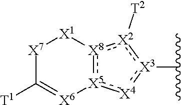

moiety is

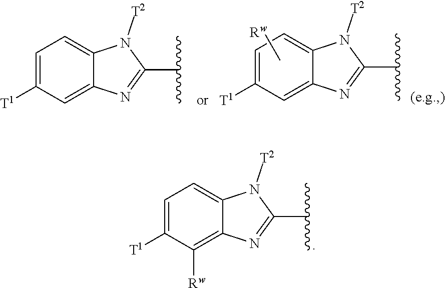

For example, the

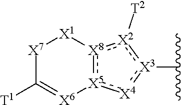

moiety can be

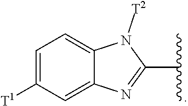

As another non-limiting example, the

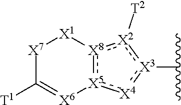

moiety can be

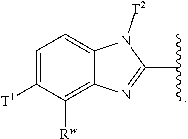

In some embodiments of Formula I, the

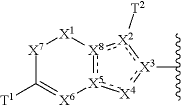

moiety is

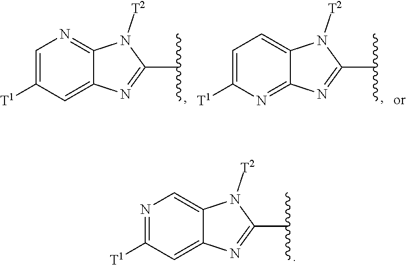

For example, the

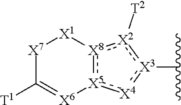

moiety can be

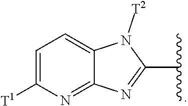

As another non-limiting example, the

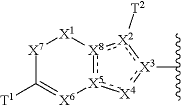

moiety can be

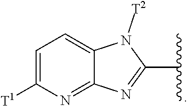

As another non-limiting example, the

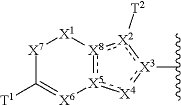

moiety can be

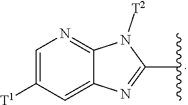

In some embodiments of Formula I, T² is (C₁-C₆)alkyl which is substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.

In some embodiments of Formula I, T² is (C₁-C₆)alkyl which is substituted with 3- to 6-membered heterocycloalkyl. In some embodiments of Formula I, T² is (C₁-C₃)alkyl which is substituted with 4- to 6-membered heterocycloalkyl. In some embodiments of Formula I, T² is (C₁-C₃)alkyl which is substituted with oxetanyl. As a non-limiting example of the foregoing embodiments, T² can be

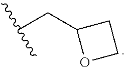 [SMILES: *CC1CCO1]

For example, T² can be

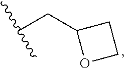 [SMILES: *CC1CCO1]

wherein the stereogenic center in T² has (S)-configuration.

In some embodiments of Formula I, L¹ is (C₁-C₃)alkylene which is optionally substituted with 1-3 R^{L}. As a non-limiting example of the foregoing embodiments, L¹ can be CH₂.

In some embodiments of Formula I, L¹ is a bond.

In some embodiments of Formula I, L² is a bond.

In some embodiments of Formula I, L¹ is CH₂; and L² is a bond.

In some embodiments of Formula I, L¹ is a bond; and L² is a bond. For avoidance of doubt, when L¹ is a bond; and L² is a bond, X³ is directly attached to Ring A.

In some embodiments of Formula I, Ring A is

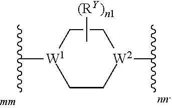

In some embodiments, W¹ is N. In some embodiments, W² is CR^{Y2}. In some embodiments, R^{Y2} is hydrogen. For example, W² can be CH. In some embodiments, W² is N. In some embodiments, n1 is 0. In some embodiments, n1 is 1.

As a non-limiting example of the foregoing embodiments, Ring A can be

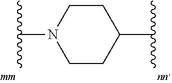 [SMILES: *C1CCN(*)CC1]

In some embodiments of Formula I, Ring A is

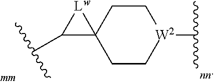

In some embodiments, L^{w} is CH₂. In some embodiments, W² is N. As a non-limiting example of the foregoing embodiments, Ring A can be

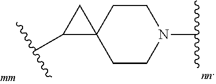 [SMILES: *C1CC12CCN(*)CC2]

In some embodiments of Formula I, Ring A is selected from the group consisting of:
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y}.

In some embodiments of Formula I, Ring A is

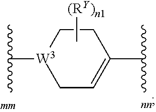

W³ is N or CH; and n1 is 0, 1, or 2. In some embodiments, n1 is 0. In some embodiments, W³ is N. In some embodiments, W³ is CH. As a non-limiting example of the foregoing embodiments, Ring A can be

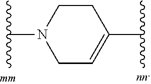 [SMILES: *C1=CCN(*)CC1]

As a further non-limiting example of the foregoing embodiments, Ring A can be

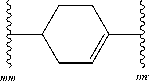 [SMILES: *C1=CCC(*)CC1]

In some embodiments of Formula I, L² is a bond; L¹ is CH₂; and Ring A is

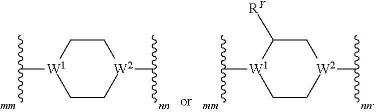

As a non-limiting example of the foregoing disclosure, Ring A can be

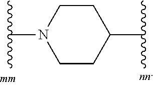 [SMILES: *C1CCN(*)CC1]

In some embodiments of Formula I, L² is a bond; L¹ is CH₂; and Ring A is

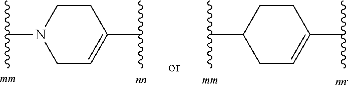 [SMILES: *C1=CCN(*)CC1 ; *C1=CCC(*)CC1]

In some embodiments of Formula I, L² is a bond; L¹ is a bond; and Ring A is

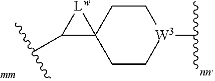

As a non-limiting example of the foregoing embodiments, Ring A can be

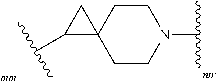 [SMILES: *C1CC12CCN(*)CC2]

In some embodiments of Formula I, B⁴ is CR¹ (e.g., CH). In some embodiments of Formula I, B⁴ is N.

In some embodiments of Formula I, B is CR¹ (e.g., CH). In some embodiments of Formula I, B¹ is N.

In some embodiments of Formula I, B³ is CR¹ (e.g., CH). In some embodiments of Formula I, B³ is N.

In some embodiments of Formula I, B² is CR¹ (e.g., CH). In some embodiments of Formula I, B² is N.

In some embodiments of Formula I, Ring B is

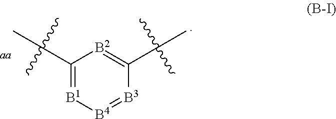

In some embodiments, B² is N. In some embodiments, B⁴ is CR¹ (e.g., CH). In some embodiments, B¹ is CR¹ (e.g., CH). In some embodiments, B³ is CR¹ (e.g., CH). In some embodiments, B² is N; and B¹, B³, and B⁴ are independently CR¹. For example, B² can be N; and B¹, B³, and B⁴ can each be CH.

In some embodiments of Formula I, Ring B is

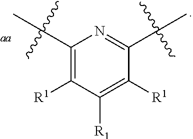

As a non-limiting example of the foregoing embodiments, Ring B can be

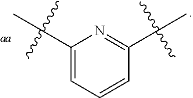 [SMILES: *c1cccc(*)n1]

In some embodiments of Formula I, Ring B is

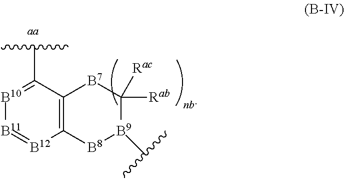

In some embodiments, B⁹ is CR^{aa} (e.g., CH or C(C₁-C₃ alkyl) such as CMe). In some embodiments, B⁷ is —O—. In some embodiments, B⁸ is —O—. In some embodiments, B⁷ is —O—; and B⁸ is —O—. In some embodiments, nb is 1. In some embodiments, R^{ab} is H. In some embodiments, R^{ac} is H. In some embodiments, R^{aa}, R^{ab}, and R^{ac} are each H. In some embodiments, R^{aa} is (C₁-C₃)alkyl; and R^{ab} and R^{ac} are H. In some embodiments, nb is 0. In some embodiments, B¹⁰ is CR¹. As a non-limiting example of the foregoing embodiments, B¹⁰ can be CH. In some embodiments, B¹¹ is CR¹. As a non-limiting example of the foregoing embodiments, B¹¹ can be CH. In some embodiments, B¹² is CR¹. As a non-limiting example of the foregoing embodiments, B² can be CH. In some embodiments, B¹⁰, B¹¹, and B¹² are independently selected CR¹. As a non-limiting example of the foregoing embodiments, B¹⁰, B¹¹, and B¹² are each CH.

In some embodiments of Formula I, Ring B is

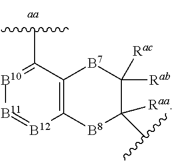

In some embodiments, B⁷ is —O—. In some embodiments, B⁸ is —O—. In some embodiments, B⁷ is —O—; and B⁸ is —O—. In some embodiments, R^{aa} is H. In some embodiments, R^{aa} is (C₁-C₃)alkyl. As a non-limiting example of the foregoing embodiments, R^{aa} can be methyl. In some embodiments, R^{ab} is H. In some embodiments, R^{ac} is H. In some embodiments, R^{aa}, R^{ab}, and R^{ac} are each H. In some embodiments, R^{aa} is (C₁-C₃)alkyl; and R^{ab} and R^{ac} are H. In some embodiments, B¹⁰ is CR¹. As a non-limiting example of the foregoing embodiments, B¹⁰ can be CH. In some embodiments, B¹¹ is CR¹. As a non-limiting example of the foregoing embodiments, B¹¹ can be CH. In some embodiments, B¹² is CR¹. As a non-limiting example of the foregoing embodiments, B¹² can be CH. In some embodiments, B¹⁰, B¹¹, and B¹² are independently selected CR¹. As a non-limiting example of the foregoing embodiments, B¹⁰, B¹¹, and B¹² are each CH. In some embodiments, the carbon atom to which L⁴ and R^{aa} are both attached has (R)-configuration. In some embodiments, the carbon atom to which L⁴ and R^{aa} are both attached has (S)-configuration.

In some embodiments of Formula I, Ring B is

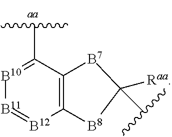

In some embodiments, B⁷ is —O—. In some embodiments, B⁸ is —O—. In some embodiments, B⁷ is —O—; and B⁸ is —O—. In some embodiments, R^{aa} is H. In some embodiments, R^{aa} is (C₁-C₃)alkyl. As a non-limiting example of the foregoing embodiments, R^{aa} can be methyl. In some embodiments, B¹⁰ is CR¹. As a non-limiting example of the foregoing embodiments, B¹⁰ can be CH. In some embodiments, B¹¹ is CR¹. As a non-limiting example of the foregoing embodiments, B¹¹ can be CH. In some embodiments, B¹² is CR¹. As a non-limiting example of the foregoing embodiments, B² can be CH. In some embodiments, B¹⁰, B¹¹, and B¹² are independently selected CR¹. As a non-limiting example of the foregoing embodiments, B¹⁰, B¹¹, and B¹² are each CH. In some embodiments, the carbon atom to which L⁴ and R^{aa} are both attached has (R)-configuration. In some embodiments, the carbon atom to which L⁴ and R^{aa} are both attached has (S)-configuration.

In some embodiments of Formula I, Ring B is

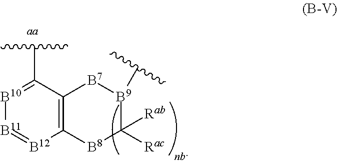

In some embodiments, B⁹ is CR^{aa} (e.g., CH or C(C₁-C₃ alkyl) such as CMe). In some embodiments, B⁷ is —O—. In some embodiments, B⁸ is —O—. In some embodiments, B⁷ is —O—; and B⁸ is —O—. In some embodiments, nb is 1. In some embodiments, R^{ab} is H. In some embodiments, R^{ac} is H. In some embodiments, R^{aa}, R^{ab}, and R^{ac} are each H. In some embodiments, R^{aa} is (C₁-C₃)alkyl; and R^{ab} and R^{ac} are H. In some embodiments, nb is 0. In some embodiments, B¹⁰ is CR¹. As a non-limiting example of the foregoing embodiments, B¹⁰ can be CH. In some embodiments, B¹¹ is CR¹. As a non-limiting example of the foregoing embodiments, B¹¹ can be CH. In some embodiments, B¹² is CR¹. As a non-limiting example of the foregoing embodiments, B¹² can be CH. In some embodiments, B¹⁰, B¹¹, and B¹² are independently selected CR¹. As a non-limiting example of the foregoing embodiments, B¹⁰, B¹¹ and B¹² are each CH.

In some embodiments of Formula I, Ring B is

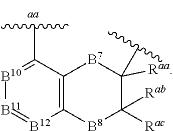

In some embodiments, B⁷ is —O—. In some embodiments, B⁸ is —O—. In some embodiments, B⁷ is —O—; and B⁸ is —O—. In some embodiments, R^{aa} is H. In some embodiments, R^{aa} is (C₁-C₃)alkyl. As a non-limiting example of the foregoing embodiments, R^{aa} can be methyl. In some embodiments, R^{ab} is H. In some embodiments, R^{ac} is H. In some embodiments, R^{aa}, R^{ab}, and R^{ac} are each H. In some embodiments, R^{aa} is (C₁-C₃)alkyl; and R^{ab} and R^{ac} are H. In some embodiments, B¹⁰ is CR¹. As a non-limiting example of the foregoing embodiments, B¹⁰ can be CH. In some embodiments, B¹¹ is CR¹. As a non-limiting example of the foregoing embodiments, B¹ can be CH. In some embodiments, B¹² is CR¹. As a non-limiting example of the foregoing embodiments, B¹² can be CH. In some embodiments, B¹⁰, B¹¹ and B¹² are independently selected CR¹. As a non-limiting example of the foregoing embodiments, B¹⁰, B¹¹, and B¹² are each CH. In some embodiments, the carbon atom to which L⁴ and R^{aa} are both attached has (R)-configuration. In some embodiments, the carbon atom to which L⁴ and R^{aa} are both attached has (S)-configuration.

In some embodiments of Formula I, Ring B is

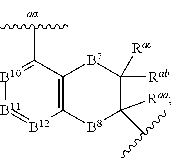

B⁷ and B⁸ are —O—; and R^{aa} is H or (C₁-C₃)alkyl. In some embodiments, B¹, B¹¹, and B¹² are each independently selected CR¹. As non-limiting examples of the foregoing embodiments, B¹⁰, B¹¹ and B¹² can be CH. In some embodiments, R^{aa} is H. In some embodiments, R^{aa} is (C₁-C₃)alkyl (e.g., methyl). In some embodiments, R^{ab} and R^{ac} are H.

In some embodiments of Formula I, Ring B is

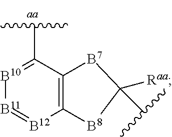

B⁷ and B⁸ are —O—; and R^{aa} is H or (C₁-C₃)alkyl. In some embodiments, B¹, B¹¹, and B¹² are each independently selected CR¹. As non-limiting examples of the foregoing embodiments, B¹⁰, B¹¹ and B¹² can be CH. In some embodiments, R^{aa} is H. In some embodiments, R^{aa} is (C₁-C₃)alkyl (e.g., methyl).

In some embodiments of Formula I, Ring B is

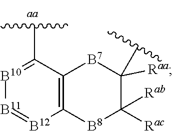

B⁷ and B⁸ are —O—; and R^{aa} is H or (C₁-C₃)alkyl. In some embodiments, B¹, B¹¹, and B¹² are each independently selected CR¹. As non-limiting examples of the foregoing embodiments, B¹⁰, B¹¹ and B¹² can be CH. In some embodiments, R^{aa} is H. In some embodiments, R^{aa} is (C₁-C₃)alkyl (e.g., methyl). In some embodiments, R^{ab} and R^{ac} are H.

As non-limiting examples, Ring B can be

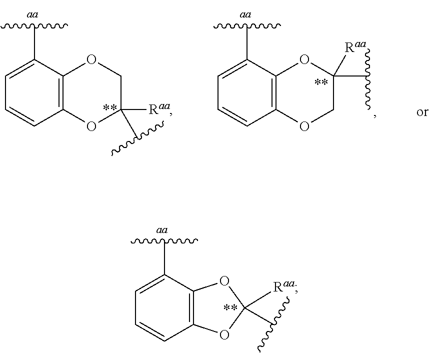

and the carbon atom labelled with ** has (R)-configuration.

As another non-limiting example, Ring B can be

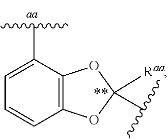

and the carbon atom labelled with ** has (S)-configuration.

In some embodiments of Formula I, L⁴ is a bond.

In some embodiments of Formula I, Ring B is (B-IV) or (B-V); and L⁴ is a bond.

In some embodiments of Formula I, L⁴ is —Z¹—Z²—*, wherein * represents the point of attachment to Ring C.

In some embodiments of Formula I, Z¹ is —O—. In some embodiments of Formula I, Z¹ is a bond.

In some embodiments of Formula I, Z² is —CH₂— optionally substituted with 1-2 R^{c}. As a non-limiting example of the foregoing embodiments, Z² can be —CH₂—.

In some embodiments, L⁴ is —O—CH₂—*. In some embodiments, L⁴ is —CH₂—.

In some embodiments of Formula I, L⁴ is —O—Z²—*, wherein * represents the point of attachment to Ring C; and Z² is —CH₂— optionally substituted with 1-2 R^{c}.

In some embodiments of Formula I, Ring C is selected from the group consisting of: phenyl, 5- to 6-membered heteroaryl, and 5- to 10-membered bicycloheteroaryl. In some embodiments, Ring C is selected from the group consisting of: phenyl and 6-membered heteroaryl. As a non-limiting example of the foregoing embodiments, Ring C can be phenyl. As another non-limiting example, Ring C can be a 6-membered heteroaryl (e.g., pyridyl).

In some embodiments of Formula I, b is 1-3. In some embodiments, b is 2. In some embodiments, b is 1. In some embodiments, b is 0.

In some embodiments of Formula I, Ring C is phenyl; and b is 2.

In some embodiments of Formula I, Ring C is phenyl; and b is 1.

In some embodiments of Formula I, Ring C is phenyl; and b is 0.

In some embodiments of Formula I, Ring C is a 6-membered heteroaryl (e.g., pyridyl); and b is 1 or 2 (e.g., 1).

In some embodiments of Formula I,

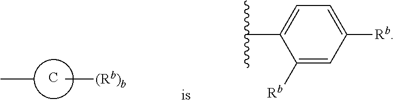

In some embodiments, each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN. In some embodiments, each occurrence of R^{b} is independently selected from the group consisting of —F, —Cl, CF₃, and CN.

In some embodiments of Formula I,

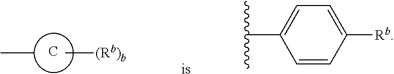

In some embodiments, R^{b} is independently selected from the group consisting of: (C₁-C₆)alkyl, (C₁-C₆)haloalkyl, (C₁-C₆)alkoxy, (C₁-C₆)haloalkoxy, halogen, CN, —C(O)NH₂, —CONH(C₁-C₃ alkyl) and C(O)N(C₁-C₃)alky)₂.

In some embodiments, R^{b} is independently selected from the group consisting of —F, —Cl, CF₃, CN, and C(O)NH₂.

In some embodiments of Formula I,

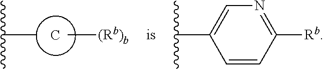

In some embodiments, each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN. In some embodiments, each occurrence of R^{b} is independently selected from the group consisting of —F, —Cl, CF₃, and CN.

In some embodiments, the compound of Formula I is a compound of Formula (I-A1) or a pharmaceutically acceptable salt thereof:

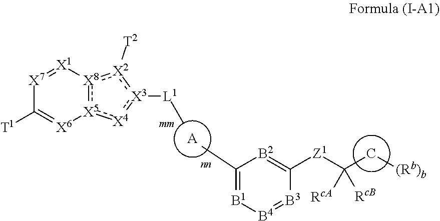

wherein R^{cA} and R^{cB} are independently selected from the group consisting of H and R.

In some embodiments of Formula (I-A1), Z¹ is —O—.

In some embodiments of Formula (I-A1), R^{cA} is H.

In some embodiments of Formula (I-A1), R^{cB} is H.

In some embodiments Formula (I-A1), the ring containing B¹, B², B³, and B⁴ is

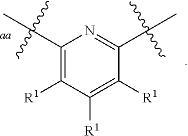

As a non-limiting example of the foregoing embodiments, the ring containing B¹, B², B³, and B⁴ can be

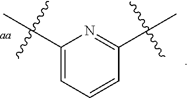 [SMILES: *c1cccc(*)n1]

In one aspect, the disclosure features compounds of Formula (I-A3):

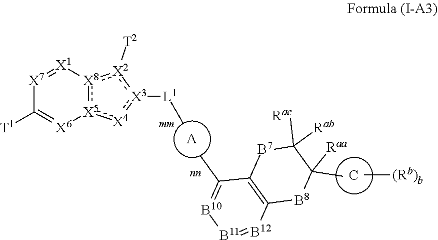

or a pharmaceutically acceptable salt thereof.

In some embodiments of Formula (I-A3), R^{ab} and R^{ac} are H.

In one aspect, the disclosure features compounds of Formula (I-A4):

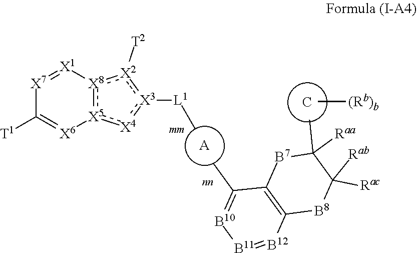

or a pharmaceutically acceptable salt thereof.

In some embodiments of Formula (I-A4), R^{ab} and R^{ac} are H.

In one aspect, the disclosure features compounds of Formula (I-A5):

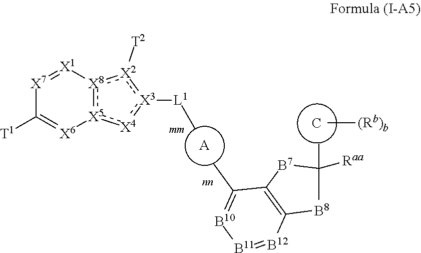

or a pharmaceutically acceptable salt thereof.

In some embodiments of Formula (I-A3), (I-A4), or (I-A5), R^{aa} is H.

In some embodiments of Formula (I-A3), (I-A4), or (I-A5), R^{aa} is (C₁-C₃)alkyl.

As a non-limiting example of the foregoing embodiments, R^{aa} can be methyl.

In some embodiments of Formula (I-A3), (I-A4), or (I-A5), B⁷ is —O—; and B⁸ is —O—.

In some embodiments of Formula (I-A3), (I-A4), or (I-A5), B¹⁰, B¹¹, and B¹² are independently selected CR¹. For example, B¹⁰, B¹¹, and B¹² can each be CH.

In some embodiments of Formula (I-A3), (I-A4), or (I-A5), the carbon to which both R^{aa} and Ring C are attached has (R)-configuration. In some embodiments of Formula (I-A3), (I-A4), or (I-A5), the carbon to which both R^{aa} and Ring C are attached has (S)-configuration.

In some embodiments of Formula (I-A3), (I-A4), or (I-A5), X³ is C; X² and X⁴ are N; and X⁵ and X⁸ are C.

In some embodiments of Formula (I-A1), (I-A3), (I-A4), or (I-A5), the

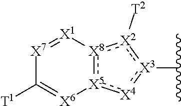

moiety is

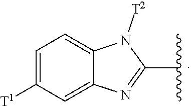

In some embodiments of Formula (I-A1), (I-A3), (I-A4), or (I-A5), the

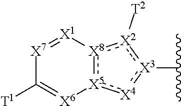

moiety is

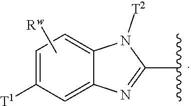

In some embodiments of Formula (I-A1), (I-A3), (I-A4), or (I-A5), the

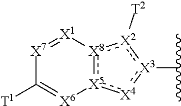

moiety is

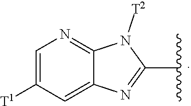

In some embodiments of Formula (I-A1), (I-A3), (I-A4), or (I-A5), the

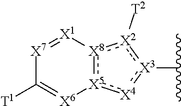

moiety is

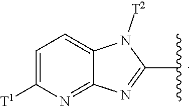

In some embodiments of Formula (I-A1), (I-A3), (I-A4), or (I-A5), the

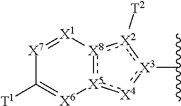

moiety is

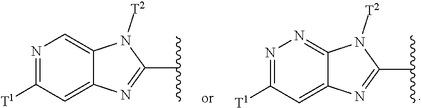

In some embodiments of Formula (I-A1), (I-A3), (I-A4), or (I-A5), T¹ is —NR^{s}C(═O)R^{z} or —NR^{s}S(O)₁₋₂—R^{z}; and R^{z} is (C₁-C₆)alkyl optionally substituted with 1-4 R^{v}. In some embodiments, R^{z} is (C₁-C₃)alkyl (e.g., methyl, ethyl, or isopropyl). In some embodiments, R^{z} is (C₁-C₃)alkyl substituted with (C₁-C₃)alkoxy (e.g., —CH₂CH₂OMe). In some embodiments, R^{z} is (C₁-C₃)alkyl substituted with 1-3 independently selected halo (e.g., —CH₂CF₃).

In some embodiments of Formula (I-A1), (I-A3), (I-A4), or (I-A5), T¹ is —NR^{s}C(═O)R^{z} or —NR^{s}S(O)₁₋₂—R^{z}; and R^{z} is —R^{z3} or —CH₂—R^{z3}. In some embodiments, R^{z3} is selected from the group consisting of: phenyl, (C₃-C₆)cycloalkyl, and 3- to 6-membered heterocycloalkyl, each optionally substituted with 1-2 R^{v}. For example, R^{z3} can be R^{z3} can be phenyl, cyclopropyl, cyclobutyl, difluorocyclobutyl (e.g.,

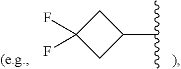 [SMILES: *C1CC(F)(F)C1]

cyclopentyl, oxetanyl

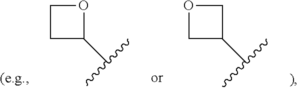 [SMILES: *C1CCO1 ; *C1COC1]

tetrahydropyranyl

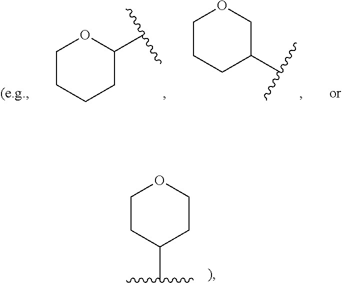 [SMILES: *C1CCCCO1 ; *C1CCCOC1 ; *C1CCOCC1]

or tetrahydro-2H-thiopyran 1,1-dioxide

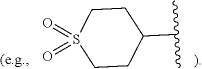 [SMILES: *C1CCS(=O)(=O)CC1]

In some embodiments, T³ is —NHC(═O)R^{z}. In some embodiments, T³ is —NHS(O)₂—R^{z}.

In some embodiments of Formula (I-A1), (I-A3), (I-A4), or (I-A5), T is —(CR^{x}R^{x})_{q}—S(O)₂R^{z}; and q is 1, 2, or 3. In some embodiments, R^{z} is (C₁-C₆)alkyl optionally substituted with 1-4 R^{v}. In some embodiments of Formula, R^{z} is (C₁-C₃)alkyl. In some embodiments, q is 1. In some embodiments, q is 2 or 3. In some embodiments, each R^{x} is hydrogen. In some embodiments, q is 1; and each R^{x} is hydrogen.

In some embodiments of Formula (I-A1), (I-A3), (I-A4), or (I-A5), T¹ is selected from the group consisting of: —C(═O)N(R^{s})R^{z}, —S(o)₁₋₂N(R^{s})R^{z}, and —S(═NR^{s})(═O)N(R^{s})R^{z}. In some embodiments, T¹ is —C(═O)N(R^{s})R^{z}. For example, T¹ can be —C(═O)NR^{z}. In some embodiments, T¹ is —S(O)₂N(R^{s})R^{z}. For example, T¹ can be —S(O)₂NR^{z}. In some embodiments, T¹ is —S(═NR^{s})(═O)N(R^{s})R^{z}. For example, T¹ can be —S(═NH)(═O)N(R^{s})R^{z} (e.g., —S(═NH)(═O)N(H)R^{z}). In some embodiments, R^{z} is hydrogen or (C₁-C₆)alkyl optionally substituted with 1-4 R^{v}. In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula (I-A1), (I-A3), (I-A4), or (I-A5), T is —N(R^{s})C(═O)N(R^{s})R^{z} or —N(R^{s})C(═O)OR^{z}. In some embodiments, T is —NHC(═O)NHR^{z}. In some embodiments, T¹ is —N(H)C(═O)OR^{z}. In some embodiments, R^{z} is hydrogen or (C₁-C₆)alkyl optionally substituted with 1-4 R^{v} In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula (I-A1), (I-A3), (I-A4), or (I-A5), T is —(CR^{x}R^{x})_{q}—C(═O)OH; and q is 1, 2, or 3. In some embodiments, q is 1. In some embodiments, q is 2. In some embodiments, each R^{x} is hydrogen. In some embodiments, one pair of R^{x} on the same carbon, taken together with the carbon atom to which each is attached, forms a (C₃-C₈)cycloalkyl ring; and each remaining R^{x} when present is hydrogen. As a non-limiting example of the foregoing embodiments, R^{x} on the same carbon, taken together with the carbon atom to which each is attached, can form a cyclopropyl ring. In some embodiments, T¹ is

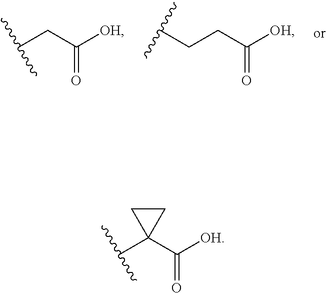 [SMILES: *CC(=O)O ; *CCC(=O)O ; *C1(C(=O)O)CC1]

In some embodiments of Formula (I-A1), (I-A3), (I-A4), or (I-A5), T¹ is C(═O)OH.

In some embodiments of Formula (I-A1), (I-A3), (I-A4), or (I-A5), T¹ is —O—(CR^{x}R^{x})_{q}—C(═O)OH; and q is 1, 2, or 3. In some embodiments, q is 1. In some embodiments, each R^{x} is hydrogen.

In some embodiments of Formula (I-A1), (I-A3), (I-A4), or (I-A5), T is —P(═O)R^{z1}R^{z2}. In some embodiments, R^{z1} and R^{z2} are independently selected (C₁-C₃)alkyl (e.g., methyl or ethyl).

In some embodiments of Formula (I-A1), (I-A3), (I-A4), or (I-A5), T¹ is 5-membered heteroaryl having 2-4 ring heteroatoms selected from the group consisting of N, O, and S, wherein the heteroaryl is optionally substituted with 1-2 R^{v}. As non-limiting examples of the foregoing embodiments, T¹ can be selected from the group consisting of:

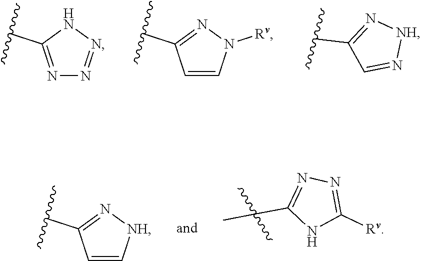

In some embodiments

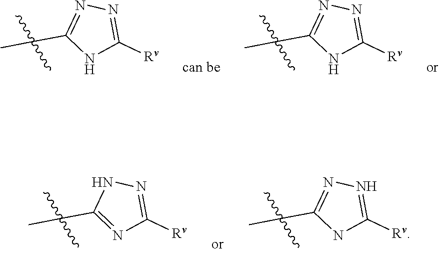

In some embodiments of Formula (I-A1), (I-A3), (I-A4), or (I-A5), T¹ is 5- to 6-membered heteroaryl that comprises an endocyclic group selected from the group consisting of:

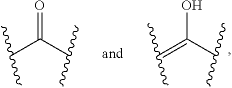

wherein the heteroaryl is further optionally substituted with 1-4 R^{v}. As non-limiting examples of the foregoing embodiments, T¹ can be selected from the group consisting of:

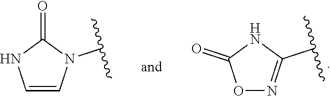 [SMILES: *n1cc[nH]c1=O ; *c1noc(=O)[nH]1]

In some embodiments of Formula (I-A1), (I-A3), (I-A4), or (I-A5), T¹ is

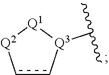

Q¹ is C(═O) or S(O)₂; Q² is O, NH, —CH₂—, or —CH₂—CH₂—; Q³ is N or CH; and is a single bond or a double bond, provided that T¹ is non-aromatic. As non-limiting examples of the foregoing embodiments, T¹ can be selected from the group consisting of:

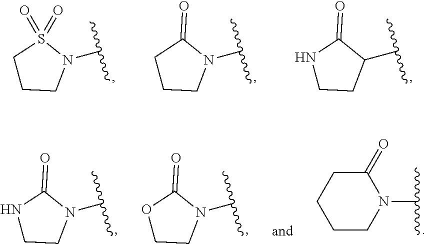

In some embodiments of Formula (I-A1), (I-A3), (I-A4), or (I-A5), T¹ is (C₁-C₆)haloalkyl substituted with —OH and further optionally substituted with 1-2 R^{v}. In some embodiments, T¹ is (C₁-C₆)haloalkyl substituted with —OH. As a non-limiting example of the foregoing embodiments, T¹ can be

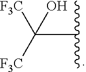 [SMILES: *C(O)(C(F)(F)F)C(F)(F)F]

In some embodiments of Formula (I-A1), (I-A3), (I-A4), or (I-A5), T² is (C₁-C₆)alkyl which is substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.

In some embodiments of Formula (I-A1), (I-A3), (I-A4), or (I-A5), T² is (C₁-C₃)alkyl which is substituted with 4- to 6-membered heterocycloalkyl.

In some embodiments of Formula (I-A1), (I-A3), (I-A4), or (I-A5), T² is (C₁-C₃)alkyl which is substituted with oxetanyl. As a non-limiting example of the foregoing embodiments, T² can be

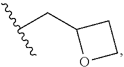 [SMILES: *CC1CCO1]

optionally wherein the stereogenic center in T² has (S)-configuration.

In some embodiments of Formula (I-A1), (I-A3), (I-A4), or (I-A5), L¹ is CH₂; and Ring A is

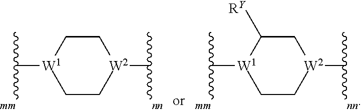

As a non-limiting example of the foregoing embodiments, Ring A can be

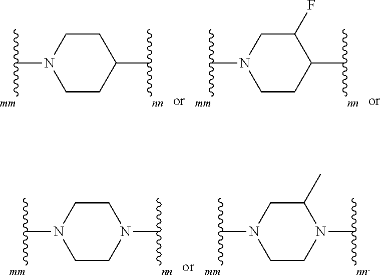

In some embodiments of Formula (I-A1), (I-A3), (I-A4), or (I-A5), L¹ is a bond; and Ring A is

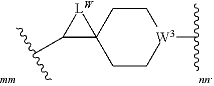

As a non-limiting example of the foregoing embodiments, Ring A can be

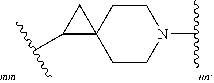 [SMILES: *C1CC12CCN(*)CC2]

In some embodiments of Formula (I-A1), (I-A3), (I-A4), or (I-A5), L¹ is CH₂; and Ring A is

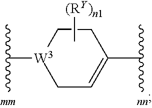

W³ is N or CH; and n1 is 0, 1, or 2. As a non-limiting example of the foregoing embodiments, Ring A can be

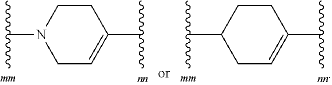 [SMILES: *C1=CCN(*)CC1 ; *C1=CCC(*)CC1]

In some embodiments of Formula (I-A1), (I-A3), (I-A4), or (I-A5), Ring C is selected from the group consisting of: phenyl and 6-membered heteroaryl; and b is 1 or 2.

In some embodiments of Formula (I-A1), (I-A3), (I-A4), or (I-A5),

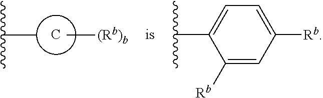

In some embodiments of Formula (I-A1), (I-A3), (I-A4), or (I-A5),

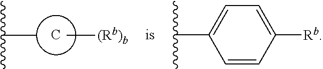

In some embodiments of Formula (I-A1), (I-A3), (I-A4), or (I-A5),

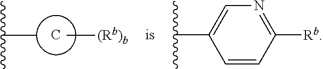

In some embodiments of Formula (I-A1), (I-A3), (I-A4), or (I-A5), each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN.

In some embodiments of Formula (I-A1), (I-A3), (I-A4), or (I-A5), each occurrence of R^{b} is independently selected from the group consisting of —F, —Cl, CF₃, and CN.

In some embodiments, the compound of Formula I is selected from the group consisting of the compounds in Table C₁ or Table C₂, or a pharmaceutically acceptable salt or solvate thereof.

Lengthy table referenced here
US20230391760A1-20231207-T00001
Please refer to the end of the specification for access instructions.

Lengthy table referenced here
US20230391760A1-20231207-T00002
Please refer to the end of the specification for access instructions.

The compounds of Formula I include pharmaceutically acceptable salts thereof. In addition, the compounds of Formula I also include other salts of such compounds which are not necessarily pharmaceutically acceptable salts, and which may be useful as intermediates for preparing and/or purifying compounds of Formula I and/or for separating enantiomers of compounds of Formula I. Non-limiting examples of pharmaceutically acceptable salts of compounds of Formula I include trifluoroacetic acid salts.

It will further be appreciated that the compounds of Formula I or their salts may be isolated in the form of solvates, and accordingly that any such solvate is included within the scope of the present invention. For example, compounds of Formula I and salts thereof can exist in unsolvated as well as solvated forms with pharmaceutically acceptable solvents such as water, ethanol, and the like.

Pharmaceutical Compositions and Administration

When employed as pharmaceuticals, the compounds of Formula I, including pharmaceutically acceptable salts or solvates thereof can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula I, or a pharmaceutically acceptable salt or solvate thereof, in combination with one or more pharmaceutically acceptable excipients (carriers). For example, a pharmaceutical composition prepared using a compound of Formula I, or a pharmaceutically acceptable salt or solvate thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula I, or a pharmaceutically acceptable salt or solvate thereof with a pharmaceutically acceptable excipient. Pharmaceutical compositions containing a compound of Formula I, or a pharmaceutically acceptable salt or solvate thereof as the active ingredient can be prepared by intimately mixing the compound of Formula I, or a pharmaceutically acceptable salt or solvate thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In some embodiments, the compound or pharmaceutical composition can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

In some embodiments, the compounds and pharmaceutical compositions described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a preferred route of administration is parenteral (e.g., intratumoral).

In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) as described herein or pharmaceutical compositions thereof can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In some embodiments, suppositories can be prepared by mixing a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) or pharmaceutical compositions as described herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) as described herein or a pharmaceutical composition thereof is formulated for local delivery to the digestive or GI tract by way of oral administration (e.g., solid or liquid dosage forms.).

In some embodiments, solid dosage forms for oral administration include capsules, tablets, pills, powders, and granules. In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG's, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) as described herein or a pharmaceutical composition thereof is formulated for ocular administration. In some embodiments, ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) as described herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

In any of the foregoing embodiments, pharmaceutical compositions as described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

In some embodiments, the dosage for a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof), is determined based on a multiple factors including, but not limited to, type, age, weight, sex, medical condition of the patient, severity of the medical condition of the patient, route of administration, and activity of the compound or pharmaceutically acceptable salt or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof), is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) as described herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) as described herein is administered as a dosage of about 100 mg/Kg.

In some embodiments, the foregoing dosages of a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof), can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) is administered to a patient for a period of time followed by a separate period of time where administration of the compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) is stopped. In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) is administered for a first period and a second period following the first period, with administration stopped during the second period, followed by a third period where administration of the compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) is started and then a fourth period following the third period where administration is stopped. For example, the period of administration of a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof) followed by a period where administration is stopped is repeated for a determined or undetermined period of time.

In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof), is orally administered to the patient one or more times per day (e.g., one time per day, two times per day, three times per day, four times per day per day or a single daily dose).

In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof), is administered by parenteral administration to the patient one or more times per day (e.g., 1 to 4 timezone time per day, two times per day, three times per day, four times per day or a single daily dose).

In some embodiments, a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof), is administered by parenteral administration to the patient weekly.

Methods of Treatment

In some embodiments, this disclosure features methods for treating a patient (e.g., a human) having a disease, disorder, or condition in which modulation of GLP-1R (e.g., repressed or impaired and/or elevated or unwanted GLP-1R) is beneficial for the treatment of the underlying pathology and/or symptoms and/or progression of the disease, disorder, or condition. In some embodiments, the methods described herein can include or further include treating one or more conditions associated, co-morbid or sequela with any one or more of the conditions described herein.

Provided herein is a method for treating a GLP-1 associated disease, disorder, or condition, the method comprising administering to a patient in need thereof an effective amount of a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof), or a pharmaceutical composition as disclosed herein.

In some embodiments, the disease, disorder, or condition includes, but is not limited to type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, and Polycystic Ovary Syndrome (PCOS).

In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

In some embodiments, the compounds and pharmaceutical compositions and methods for treating a patient described herein induce one or more of blood glucose reduction (e.g., reduce blood glucose levels), reduce blood hemoglobin A1c (HbA1c) levels, promote insulin synthesis, stimulate insulin secretion, increase the mass of 0-cells, modulate gastric acid secretion, modulate gastric emptying, decrease the body mass index (BMI), and/or decrease glucagon production (e.g., level). In certain embodiments, the compounds and pharmaceutical compositions and methods for treating a patient described herein stabilize serum glucose and serum insulin levels (e.g., serum glucose and serum insulin concentrations). Also provided herein are methods for modulating glucose or insulin levels in a patient in need of such modulating, the method comprising administering to the patient an effective amount of a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof), or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount of a compound of Formula I, or a pharmaceutically acceptable salt or solvate thereof (e.g., a compound of any one of Formulas (I-A1), (I-A3), (I-A4), and (I-A5), or a pharmaceutically acceptable salt or solvate thereof), or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

Indications

Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, and metabolic syndrome. In some embodiments, the chemical compound and pharmaceutical compositions described herein can be used to treat patients exhibiting symptoms of both obesity and insulin deficiency.

Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

Provided herein is a method of treating a diabetes mellitus in a patient, the method comprising (a) determining that the patient has type 2 diabetes mellitus, and (b) administering to the patient a therapeutically effective amount of a compound of Formula I, or a pharmaceutically acceptable salt or solvate thereof (e.g., a compound of any one of a compound of any one of Formulas (I-A1), (I-A3), (I-A4), and (I-A5), or a pharmaceutically acceptable salt or solvate thereof, or a pharmaceutically acceptable salt or solvate thereof) or a pharmaceutical composition as disclosed herein.

Provided herein is a method for treating type 2 diabetes mellitus in a patient, the method comprising administering to a patient identified or diagnosed as having type 2 diabetes mellitus a therapeutically effective amount of a compound of Formula I, or a pharmaceutically acceptable salt or solvate thereof (e.g., a compound of any one of a compound of any one of Formulas (I-A1), (I-A3), (I-A4), and (I-A5), or a pharmaceutically acceptable salt or solvate thereof, or a pharmaceutically acceptable salt or solvate thereof), or a pharmaceutical composition as disclosed herein.

Also provided herein is a method of treating type 2 diabetes mellitus in a patient in need thereof, the method comprising administering to the patient a therapeutically effective amount of a compound of Formula I, or a pharmaceutically acceptable salt or solvate thereof (e.g., a compound of any one of a compound of any one of Formulas (I-A1), (I-A3), (I-A4), and (I-A5), or a pharmaceutically acceptable salt or solvate thereof, or a pharmaceutically acceptable salt or solvate thereof), or a pharmaceutical composition as disclosed herein.

In some embodiments, the compounds and pharmaceutical compositions and methods for treating a patient with a condition, disease, or disorder (e.g., type 2 diabetes mellitus) described herein reduce fasting plasma glucose levels. In some embodiments, the compounds and pharmaceutical compositions and methods for treating a patient with a condition, disease, or disorder (e.g., type 2 diabetes mellitus) described herein reduce non-fasting plasma glucose levels. In some embodiments, the compounds and pharmaceutical compositions and methods for treating a patient with a condition, disease, or disorder (e.g., type 2 diabetes mellitus) described herein reduce HbA1c levels. In some embodiments, the compounds and pharmaceutical compositions and methods for treating a patient with a condition, disease, or disorder (e.g., type 2 diabetes mellitus) described herein reduce glucagon levels. In some embodiments, the compounds and pharmaceutical compositions and methods for treating a patient with a condition, disease, or disorder (e.g., type 2 diabetes mellitus) described herein increase insulin levels. In some embodiments, the compounds and pharmaceutical compositions and methods for treating a patient with a condition, disease, or disorder (e.g., type 2 diabetes mellitus) described herein reduce BMI.

In some embodiments, a reduction in fasting plasma glucose levels of about 5% to about 95% indicates treatment of type 2 diabetes mellitus. In some embodiments, a reduction in fasting plasma glucose levels of about 15% to about 80% indicates treatment of type 2 diabetes mellitus. In some embodiments, a reduction in fasting plasma glucose levels of about 25% to about 60% indicates treatment of type 2 diabetes mellitus. In some embodiments, a reduction in fasting plasma glucose levels to about or below 126 mg/dL, about or below 110 mg/dL, or about or below 90 mg/dL indicates treatment of the type 2 diabetes mellitus.

In some embodiments, a reduction in non-fasting plasma glucose levels of about 5% to about 95% indicates treatment of type 2 diabetes mellitus. In some embodiments, a reduction in non-fasting plasma glucose levels of about 15% to about 80% indicates treatment of type 2 diabetes mellitus. In some embodiments, a reduction in non-fasting plasma glucose levels of about 25% to about 60% indicates treatment of type 2 diabetes mellitus. In some embodiments, a reduction in non-fasting plasma glucose levels to about or below 200 mg/dL, about or below 150 mg/dL, or about or below 130 mg/dL indicates treatment of type 2 diabetes mellitus.

In some embodiments, a reduction in HbA1c levels of about 5% to about 95% indicates treatment of type 2 diabetes mellitus. In some embodiments, a reduction in HbA1c levels of about 15% to about 80% indicates treatment of type 2 diabetes mellitus. In some embodiments, a reduction in HbA1c levels of about 25% to about 60% indicates treatment of type 2 diabetes mellitus. In some embodiments, reduction in HbA1c levels to about or below 6.5%, about or below 6.0%, or about or below 5.0% indicates treatment of type 2 diabetes mellitus.

In some embodiments, a reduction in glucagon levels of about 5% to about 95% indicates treatment of type 2 diabetes mellitus. In some embodiments, a reduction in glucagon levels of about 15% to about 80% indicates treatment of type 2 diabetes mellitus. In some embodiments, a reduction in glucagon levels of about 25% to about 60% indicates treatment of type 2 diabetes mellitus. In some embodiments, an increase in insulin levels of about 5% to about 95% indicates treatment of type 2 diabetes mellitus. In some embodiments, an increase in insulin levels of about 15% to about 80% indicates treatment of type 2 diabetes mellitus. In some embodiments, an increase in insulin levels of about 25% to about 60% indicates treatment of type 2 diabetes mellitus.

In some embodiments, a reduction in BMI of about 5% to about 95% indicates treatment of type 2 diabetes mellitus. In some embodiments, a reduction in BMI of about 15% to about 80% indicates treatment of the type 2 diabetes mellitus. In some embodiments, a reduction in BMI of about 25% to about 60% indicates treatment of type 2 diabetes mellitus. In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of type 2 diabetes mellitus. In some embodiments, a reduction in BMI to about or below 40, about or below 30, or about or below 20 indicates treatment of type 2 diabetes mellitus.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are also useful in improving the therapeutic effectiveness of metformin.

Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolmans disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and may ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl:S186-90). In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

Other non-limiting examples of disorders in metabolically important tissues include joint disorders (e.g., osteoarthritis, secondary osteoarthritis), steatosis (e.g. in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or haemodialysis, bone fracture, bone surgery, aging, pregnancy, protection against bone fractures, and malnutritionpolycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, the compounds and pharmaceutical compositions described herein can be used for treating surgical trauma by improving recovery after surgery and/or by preventing the catabolic reaction caused by surgical trauma.

Cardiovascular and Vascular Diseases

In some embodiments, the condition, disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the condition, disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

Neurological Diseases

In some embodiments, the condition, disease or disorder is a neurological disorder (e.g., neurodegenerative disorder) or a psychiatric disorder. Non-limiting examples of neurological disorders include brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., US20060275288A1.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., US20120021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson.

Insulin-Related

In some embodiments, the condition, disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidaemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperuricacidemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

Autoimmune Disorders

In some embodiments, the condition, disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves disease. See, e.g., US20120148586A1.

Stomach and Intestine-Related Disorders

In some embodiments, the condition, disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking.

In some embodiments, the condition, disease or disorder is an eating disorder, such as hyperphagia, binge eating, bulimia, or compulsive eating.

Inflammatory Diseases

In some embodiments, the condition, disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

Cancer

In some embodiments, the condition, disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

Hypothalamic-Pituitary Disorders

In some embodiments, the condition, disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and Cushing's disease.

In some embodiments, the condition, disease or disorder associated with diabetes is related to the hypothalamic-pituitary-gonadal axis.

Pulmonary Disease

In some embodiments, the condition, disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the condition, disease or disorder associated with diabetes is a pulmonary disease.

Combination Therapy

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, the compounds of X, or a pharmaceutically acceptable salt or solvate thereof as described herein can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, therapeutic agents for dysuria, and anti-emetic agents.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498), orexin receptor antagonists, melanocortin 4 receptor agonists, 11β-hydroxysteroid dehydrogenase inhibitors (e.g., AZD-4017, BVT-3498, INCB-13739), pancreatic lipase inhibitors (e.g., orlistat, cetilistat), β3 agonists (e.g., N-5984), diacylglycerol acyltransferase 1 (DGAT1) inhibitors, acetylCoA carboxylase (ACC) inhibitors, stearoyl-CoA desaturated enzyme inhibitors, microsomal triglyceride transfer protein inhibitors (e.g., R-256918), sodium-glucose cotransporter 2 (SGLT-2) inhibitors (e.g., JNJ-28431754, dapagliflozin, AVE2268, TS-033, YM543, TA-7284, ASP1941, remogliflozin), NFK inhibitors (e.g., HE-3286), PPAR agonists (e.g., GFT-505, DRF-11605, gemfibrozil and fenofibrate), phosphotyrosine phosphatase inhibitors (e.g., sodium vanadate, trodusquemin), GPR119 agonists (e.g., PSN-821, MBX-2982, APD597), glucokinase activators (e.g., piragliatin, AZD-1656, AZD6370, TTP-355, compounds described in WO006/112549, WO007/028135, WO008/047821, WO008/050821, WO008/136428 and WO008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), farnesoid X receptor (FXR) agonist, phentermine, zonisamide, norepinephrine/dopamine reuptake inhibitor, GDF-15 analog, methionine aminopeptidase 2 (MetAP2) inhibitor, diethylpropion, phendimetrazine, benzphetamine, fibroblast growth factor receptor (FGFR) modulator, and AMP-activated protein kinase (AMPK) activator.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, gliclazide, glimepiride, tolbutamide, glibenclamide, gliclazide, acetohexamide, glyclopyramide, glybuzole, glyburide), thiazolidinedione agents (e.g. rosiglitazone or pioglitazone), α-glucosidase inhibitors (e.g., voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-1/GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, B11356, GRC8200, MP-513, PF-00734200, PHX1149, SK-0403, ALS2-0426, TA-6666, TS-021, KRP-104, trelagliptin).

In some embodiments, the one or more additional therapeutic agents include those useful, for example, for treating NAFL and NASH. Non-limiting examples include FXR agonists, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21), a niacin analogue, a leukotriene D4 (LTD4) receptor antagonist, an afcetyl-CoA carboxylase (ACC) inhibitor, a ketohexokinase (KHK) inhibitor, an apoptosis signal-regulating kinase 1 (ASK1) inhibitor, an ileal bile acid transporter (IBAT) inhibitor, glycyrrhizin, schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and apoptosis signal regulating kinase-1 (ASK-1) inhibitors.

In some embodiments, the one or more additional therapeutic agents include those useful, for example, for treating hyperlipidemia. Non-limiting examples include HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4, 1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (γ-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, enalapril, delapril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and_β-blockers (e.g., metoprolol, atenolol, propranolol, carvedilol, pindolol).

In some embodiments, the one or more additional therapeutic agents include those useful, for example, as diuretics. Non-limiting examples include_xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran) FXa inhibitors (e.g., rivaroxaban, apixaban, edoxaban, YM150, compounds described in WO02/06234, WO2004/048363, WO2005/030740, WO2005/058823, and WO2005/113504) thrombolytic agents (e.g., urokinase, tisokinase, alteplase, nateplase, monteplase, pamiteplase), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-CoA reductase inhibitors (statins, e.g. rosuvastatin), cholesterol-lowering agents, bile acid sequestrants (e.g., cholestyramine), cholesterol absorption inhibitors (e.g. plant sterols such as phytosterols), cholesteryl ester transfer protein (CETP) inhibitors, inhibitors of the ileal bile acid transport system (IBAT inhibitors), bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone), centrally acting adrenergic drugs, such as central alpha agonists (e.g. clonidine), diuretic agents (e.g. furosemide), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin)), adenosine diphosphate (ADP) receptor inhibitors (e.g. clopidogrel), phosphodiesterase inhibitors (e.g. cilostazol), glycoprotein IIB/IIA inhibitors (e.g. tirofiban), adenosine reuptake inhibitors (e.g. dipyridamole), noradrenergic agents (e.g. phentermine), serotonergic agents (e.g. sibutramine), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in WO007/013694, WO2007/018314, WO2008/093639 and WO2008/099794, GPR40 agonists (e.g., fasiglifam or a hydrate thereof, compounds described in WO2004/041266, WO2004/106276, WO2005/063729, WO2005/063725, WO2005/087710, WO2005/095338, WO2007/013689 and WO2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor α (PPARα), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., mexiletine), acetylcholine receptor ligands (e.g., ABT-594), endothelin receptor antagonists (e.g., ABT-627), narcotic analgesics (e.g., morphine), a2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), and metaglidasen, AMG-131, balaglitazone, MBX-2044, rivoglitazone, aleglitazar, chiglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide.

In some embodiments, the one or more additional therapeutic agents include those useful, for example, as anti-emetic agents. As used herein, an “anti-emetic” agent refers to any agent that counteracts (e.g., reduces or removes) nausea or emesis (vomiting). While not wishing to be bound by theory, it is believed that administering one or more anti-emetic agents in combination with the formula (I) compounds described herein may allow higher dosages of the formula (I) compounds to be administered, e.g., because the patient may be able to have a normal food intake and thereby respond faster to the treatment.

Non-limiting examples of anti-emetic agents include 5HT3-receptor antagonists (serotonin receptor antagonists), neuroleptics/anti-psychotics, antihistamines, anticholinergic agents, steroids (e.g., corticosteroids), NK1-receptor antagonists (e.g., Neurokinin 1 substance P receptor antagonists), antidopaminergic agents/dopamine receptor antagonists, benzodiazepines, cannabinoids.

For example, the anti-emetic agent can be selected from the group consisting of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In some embodiments, the anti-emetic agent is a 5HT3-receptor antagonist (serotonin receptor antagonist). Non-limiting examples of 5HT3-receptor antagonists (serotonin receptor antagonists) include: Granisetron (Kytril), Dolasetron, Ondansetron (Zofran), Tropisetron, Ramosetron, Palonosetron, Alosetron, azasetron, Bemesetron, Zatisetron, Batanopirde, MDL-73147EF; Metoclopramide, N-3389 (endo-3,9-dimethyl-3,9-diazabicyclo[3,3,1]non-7-yl-1H-indazole-3-carboxamide dihydrochloride), Y-25130 hydrochloride, MDL 72222, Tropanyl-3,5-dimethylbenzoate, 3-(4-Allylpiperazin-1-yl)-2-quinoxalinecarbonitrile maleate, Zacopride hydrochloride, and Mirtazepine. Other non-limiting examples of 5HT3-receptor antagonists (serotonin receptor antagonists) include: cilansetron, clozapine, cyproheptadine, dazopride, hydroxyzine, lerisetron, metoclopramide, mianserin, olanzapine, palonosetron (+netupitant), quetiapine, qamosetron, ramosteron, ricasetron, risperidone, ziprasidone, and zatosetron.

In certain embodiments, the 5HT-3-receptor antagonist is Granisetron, Dolasetron, Ondansetron hydrochloride, Tropisetron, Ramosetron, Palonosetron, Alosetron, Bemesetron, Zatisetron, Batanopirde, MDL-73147EF, Metoclopramide, N-3389, Y-25130 hydrochloride, MDL 72222, Tropanyl-3,5-dimethylbenzoate 3-(4-AIIyI-piperazin-1-yl)-2-quinoxalinecarbonitrile maleate, Zacopride hydrochloride and Mirtazepine.

In certain embodiments, the 5HT-3-receptor antagonist is Granisetron, Dolasetron, Ondansetron hydrochloride, Tropisetron, Ramosetron, Palonosetron, Alosetron, Bemesetron, and Zatisetron.

In certain embodiments, the 5HT-3-receptor antagonist is Granisetron, Dolasetron and Ondansetron.

In certain embodiments, the 5HT-3-receptor antagonist is Granisetron.

In certain embodiments, the 5HT-3-receptor antagonist is Ondansetron.

In some embodiments, the anti-emetic agent is an antihistamine. Non-limiting examples of antihistamines include: piperazine derivatives (e.g., cyclizine, meclizine, and cinnarizine); Promethazine; Dimenhydrinate (Dramamine, Gravol); Diphenhydramine; Hydroxyzine; Buclizine; and Meclizine hydrochloride (Bonine, Antivert), doxylamine, and mirtazapine.

In some embodiments, the anti-emetic agent is an anticholinergic agent (Inhibitors of the acetylcholine receptors). Non-limiting examples of anticholinergic agents include: atropine, Scopolamine, Glycopyrron, Hyoscine, Artane (Trihexy-5 trihexyphenidyl hydrochloride), Cogentin (benztropine mesylate), Akineton (biperiden hydrochloride), Disipal (Norflex orphenadrine citrate), diphenhydramine, hydroxyzine, hyoscyamine, and Kemadrin (procyclidine hydrochloride).

In some embodiments, the anti-emetic agent is a steroid (e.g., a corticosteroid). Non-limiting examples of steroids include: betamethasone, Dexamethasone, Methylprednisolone, Prednisone®, and Trimethobenzamide (Tigan).

In some embodiments, the anti-emetic agent is an NK1-receptor antagonists (e.g., Neurokinin 1 substance P receptor antagonists). Non-limiting examples of NK1-receptor antagonists include: aprepitant, casopitant, ezlopitant, fosaprepitant, maropitant, netupitant, rolapitant, and vestipitant.

Other non-limiting examples of NK1-receptor antagonists include: MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-1, CJ-11974 j. Benserazide and carbidopa k. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g] [1,7]naphthyridine-6,13-dione], PD 154075, ([(2-benzofuran)-CH2OCO]—(R)-alpha-MeTrp-(S)—NHCH(CH3) Ph), FK888, and (D-Pro4, D-Trp7,9,10, Phe11)SP4-11.

In some embodiments, the anti-emetic agent is an anti-dopaminergic agents/dopamine receptor antagonist (e.g., dopamine receptor antagonist, e.g., D2 or D3 antagonists). Non-limiting examples include phenothiazines (e.g., promethazine, chlorpromazine, prochlorperazine, perphenazine, hydroxyzine, thiethylperazine, metopimazine,); benzamides (e.g., Metoclopramide, domperidone), butyrophenones (e.g., haloperidol, droperidol); alizapride, bromopride, clebopride, domperidone, itopride, metoclopramide, trimethobenzamide, and amisulpride.

In some embodiments, the anti-emetic agent is a non-psychoactive cannabinoids (e.g., Cannabidiol (CBD), Cannabidiol dimethylheptyl (CBD-DMH), Tetra-hydro-cannabinol (THC), Cannabinoid agonists such as WIN 55-212 (a CB1 and CB2 receptor agonist), Dronabinol (Marinol®), and Nabilone (Cesamet)).

Other exemplary anti-emetic agents include: c-9280 (Merck); benzodiazepines (diazepam, midazolam, lorazepam); neuroleptics/anti-psychotics (e.g., dixyrazine, haloperidol, and Prochlorperazine (Compazine®)); cerium oxalate; propofol; sodium citrate; dextrose; fructose(Nauzene); orthophosphoric acid; fructose; glucose (Emetrol); bismuth subsalicylate (Pepto Bismol); ephedrine; vitamin B6; peppermint, lavender, and lemon essential oils; and ginger.

Still other exemplary anti-emetic agents include those disclosed in US 20120101089A1; U.S. Pat. No. 10,071,088 B2; U.S. Pat. No. 6,673,792 B1; U.S. Pat. No. 6,197,329 B1; U.S. Pat. No. 10,828,297 B2; U.S. Pat. No. 10,322,106 B2; U.S. Pat. No. 10,525,033 B2; WO 2009080351 A1; WO 2019203753 A2; WO 2002020001 A2; U.S. Pat. No. 8,119,697 B2; U.S. Pat. No. 5,039,528; US20090305964A1; and WO 2006/111169, each of which is incorporated by reference in its entirety.

In some embodiments, the additional therapeutic agent or regimen is administered to the patient prior to contacting with or administering the compounds and pharmaceutical compositions (e.g., about one hour prior, or about 6 hours prior, or about 12 hours prior, or about 24 hours prior, or about 48 hours prior, or about 1 week prior, or about 1 month prior).

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form. As another example, the additional therapeutic agent or regimen and the compounds and pharmaceutical compositions are provided to the patient concurrently in separate dosage forms.

Patient Selection

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has a disease, disorder, or condition as provided here (e.g., a GLP-1 associated disease, disorder, or condition).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin A1c (HbA1c), fasting plasma glucose, non-fasting plasma glucose, or any combination thereof. In some embodiments, the level of HbA1c is about 6.5% to about 24.0%. In some embodiments, the level of HbA1c is greater than or about 6.5%. In some embodiments, the level of HbA1c is greater than or about 8.0%. In some embodiments, the level of HbA1c is greater than or about 10.0%. In some embodiments, the level of HbA1c is greater than or about 12.0%. In some embodiments, the level of HbA1c is greater than or about 14.0%. In some embodiments, the level of HbA1c is greater than or about 16.0%. In some embodiments, the level of HbA1c is greater than or about 18.0%. In some embodiments, the level of HbA1c is greater than or about 20.0%. In some embodiments, the level of HbA1c is greater than or about 22.0%. In some embodiments, the level of HbA1c is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient's BMI. In some embodiments, the BMI of the patient is greater than or about 22 kg/m² to greater than or about 100 kg/m². In some embodiments, the BMI of the patient is greater than or about 30 kg/m² to greater than or about 90 kg/m². In some embodiments, the BMI of the patient is greater than or about 40 kg/m² to greater than or about 80 kg/m². In some embodiments, the BMI of the patient is greater than or about 50 kg/m² to greater than or about 70 kg/m².

In some embodiments, additional factors (e.g. risk factors) used for determining if the patient has type 2 diabetes mellitus further includes age and ethnicity of the patient. In some embodiments, the patient's age is greater than or about 10 years. In some embodiments, the patient's age is greater than or about 15 years. In some embodiments, the patient's age is greater than or about 20 years. In some embodiments, the patient's age is greater than or about 25 years. In some embodiments, the patient's age is greater than or about 30 years. In some embodiments, the patient's age is greater than or about 35 years. In some embodiments, the patient's age is greater than or about 40 years. In some embodiments, the patient's age is greater than or about 42 years. In some embodiments, the patient's age is greater than or about 44 years. In some embodiments, the patient's age is greater than or about 46 years. In some embodiments, the patient's age is greater than or about 48 years. In some embodiments, the patient's age is greater than or about 50 years. In some embodiments, the patient's age is greater than or about 52 years. In some embodiments, the patient's age is greater than or about 54 years. In some embodiments, the patient's age is greater than or about 56 years. In some embodiments, the patient's age is greater than or about 58 years. In some embodiments, the patient's age is greater than or about 60 years. In some embodiments, the patient's age is greater than or about 62 years. In some embodiments, the patient's age is greater than or about 64 years. In some embodiments, the patient's age is greater than or about 66 years. In some embodiments, the patient's age is greater than or about 68 years. In some embodiments, the patient's age is greater than or about 70 years. In some embodiments, the patient's age is greater than or about 72 years. In some embodiments, the patient's age is greater than or about 74 years. In some embodiments, the patient's age is greater than or about 76 years. In some embodiments, the patient's age is greater than or about 78 years. In some embodiments, the patient's age is greater than or about 80 years. In some embodiments, the patient's age is greater than or about 85 years. In some embodiments, the patient's age is greater than or about 90 years. In some embodiments, the patient's age is greater than or about 95 years. In some embodiments, the ethnicity of the patient may be African American, American Indian or Alaska Native, Asian American, Hispanics or Latinos, or Native Hawaiian or Pacific Islander.

### EXAMPLES

The invention is further described in the following examples, which do not limit the scope of the invention described in the claims.

General information: All evaporations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (100-200 mesh). Solvent systems were reported as mixtures by volume. NMR spectra were recorded on a Bruker 400 or Varian (400 MHz) spectrometer. ¹H chemical shifts are reported in 6 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on SHIMADZU LC20-MS2020 or Agilent 1260 series 6125B mass spectrometer or Agilent 1200 series, 6110 or 6120 mass spectrometer with electrospray ionization and excepted as otherwise indicated.

### Example 1: (S)—N-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)acetamide (Compound 101a)

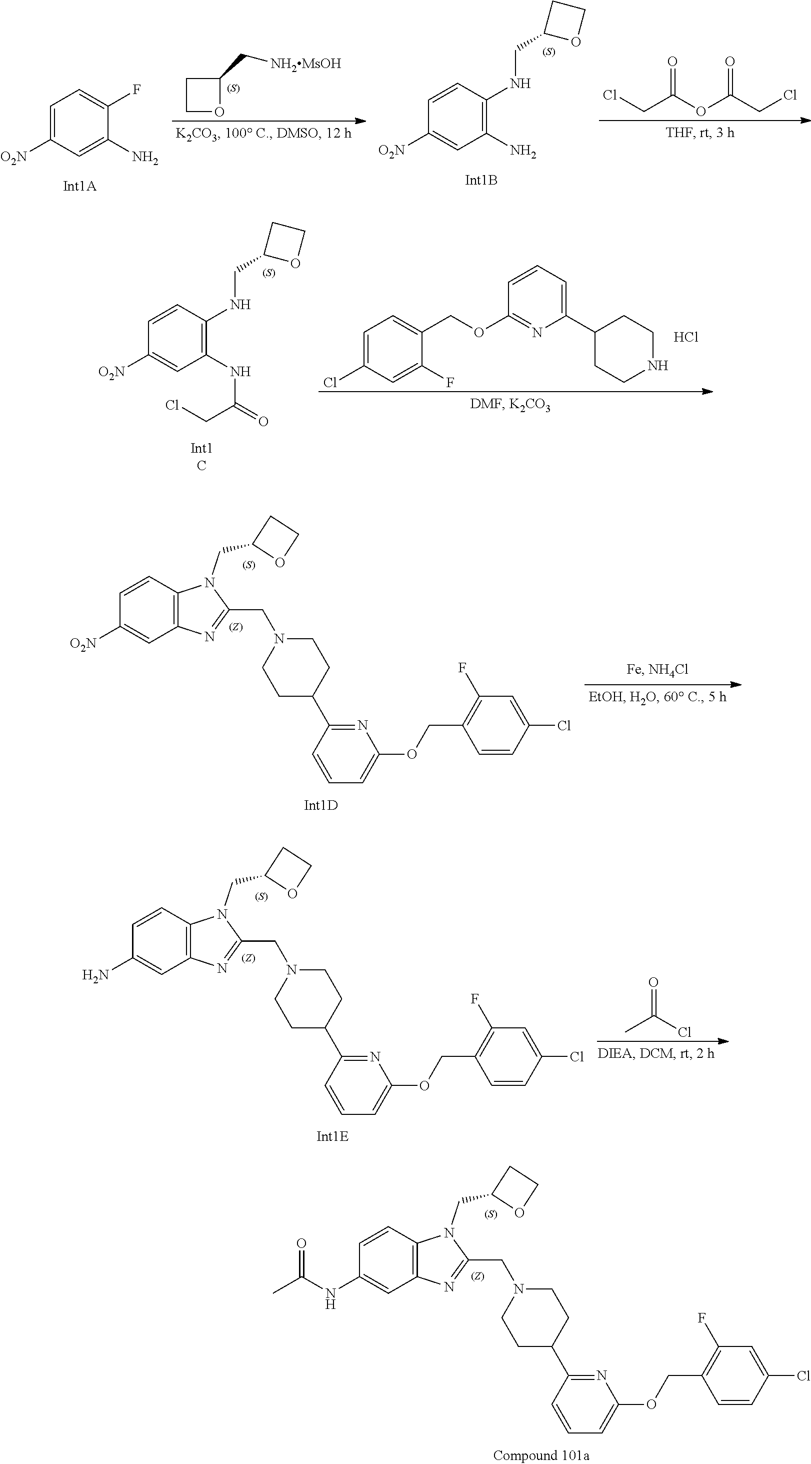

### Step A: (S)-4-nitro-N¹-(oxetan-2-ylmethyl)benzene-1,2-diamine (Int1B)

To a solution of Int1A (500 mg, 3.2 mmol) and (S)-oxetan-2-ylmethanamine MsOH salt (586 mg, 3.2 mmol) in DMSO (5 mL) is added K₂CO₃ (1.33 g, 9.6 mmol) at room temperature. The reaction is stirred at 100° C. for 12 hours. After the reaction is complete, the reaction is quenched with water (50 mL) and extracted with ethyl acetate (100 mL×3). The organic layers are combined and washed with brine (100 mL×2), dried over sodium sulfate, filtered, and concentrated in vacuo. The residue is purified by column chromatography to give Int1B (120 mg, 16.8% yield) as an orange solid. MS Calcd.: 223.1; MS Found: 224.0 [M+H]⁺.

### Step B: (S)-2-chloro-N-(5-nitro-2-((oxetan-2-ylmethyl)amino)phenyl)acetamide (Int1C)

To a solution of Int1B (120 mg, 0.53 mmol) in THE (1.5 mL) is added 2-chloroacetic anhydride (89 mg, 0.53 mmol). The reaction is stirred at room temperature for 3 hours. After the reaction is complete, the reaction is concentrated under vacuum to give Int1C (90 mg, crude) as a grey solid. MS Calcd.: 299.07; MS Found: 300.1 [M+H]⁺.

### Step C: (S)-2-((4-(6-((4-chloro-2-fluorobenzyl)oxy)pyridin-2-yl)piperidin-1-yl)methyl)-5-nitro-1-(oxetan-2-ylmethyl)-1H-benzo[d]imidazole (Int1D)

To a solution of 2-((4-chloro-2-fluorobenzyl)oxy)-6-(piperidin-4-yl)pyridine HCl salt (140 mg, 0.4 mmol), K₂CO₃ (164 mg, 1.19 mmol) in THE (1.5 mL) is added Int1C (90 mg, crude). The reaction is stirred at room temperature for 2 hours. Then the reaction is stirred at 60° C. for 12 hours. After the reaction is complete, the reaction is filtered. The filtrate is purified by column chromatography to give Int1D (43 mg, 18% yield in two steps) as a grey solid. MS Calcd.: 565.19; MS Found: 566.1 [M+H]⁺.

### Step D: (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (Int1E)

A mixture of Int1D (43 mg, 0.076 mmol) and iron powder (42 mg, 0.76 mmol) in THE (1 mL) is added NH₄Cl aqueous solution (20 mg of NH₄Cl dissolved in 0.2 mL of H₂O), The reaction is stirred at 60° C. for 5 hours. After the reaction is complete, the reaction is concentrated under vacuum to give crude Int1E as a grey solid. MS Calcd.: 535.2; MS Found: 536.2[M+H]⁺.

### Step E: (S)—N-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)acetamide (Compound 101a)

To a solution of Int1E (20 mg, crude) and DIEA (14 mg, 0.111 mmol) in DCM (1 mL) is added acetyl chloride (3.0 mg, 0.037 mmol) (missing the volume of DIEA). The reaction is stirred at room temperature for 2 hours. After the reaction is complete, the reaction mixture is purified by prep-HPLC to give Compound 101a (8 mg) as a white solid. MS Calcd.: 577.2; MS Found: 578.1 [M+H]⁺.

¹H NMR (400 MHz, CD₃OD): δ 7.95 (d, J=1.6, 1H), 7.58-7.54 (m, 2H), 7.48 (t, J=8.0, 1H), 7.39-7.42 (m, 1H), 7.16-7.22 (m, 2H), 6.82 (d, J=7.2, 1H), 6.63 (d, J=7.6, 1H), 5.41 (s, 2H), 5.21-5.27 (m, 1H), 4.75-4.81 (m, 1H), 4.59-4.67 (m, 2H), 4.41-4.47 (m, 1H), 3.92-4.05 (m, 2H), 3.00-3.12 (m, 2H), 2.73-2.82 (m, 1H), 2.62-2.68 (m, 1H), 2.48-2.55 (m, 1H), 2.31-2.43 (m, 2H), 2.14 (s, 3H), 1.84-1.92 (m, 4H)

### Example 2: (S)—N-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)propionamide (Compound 102a)

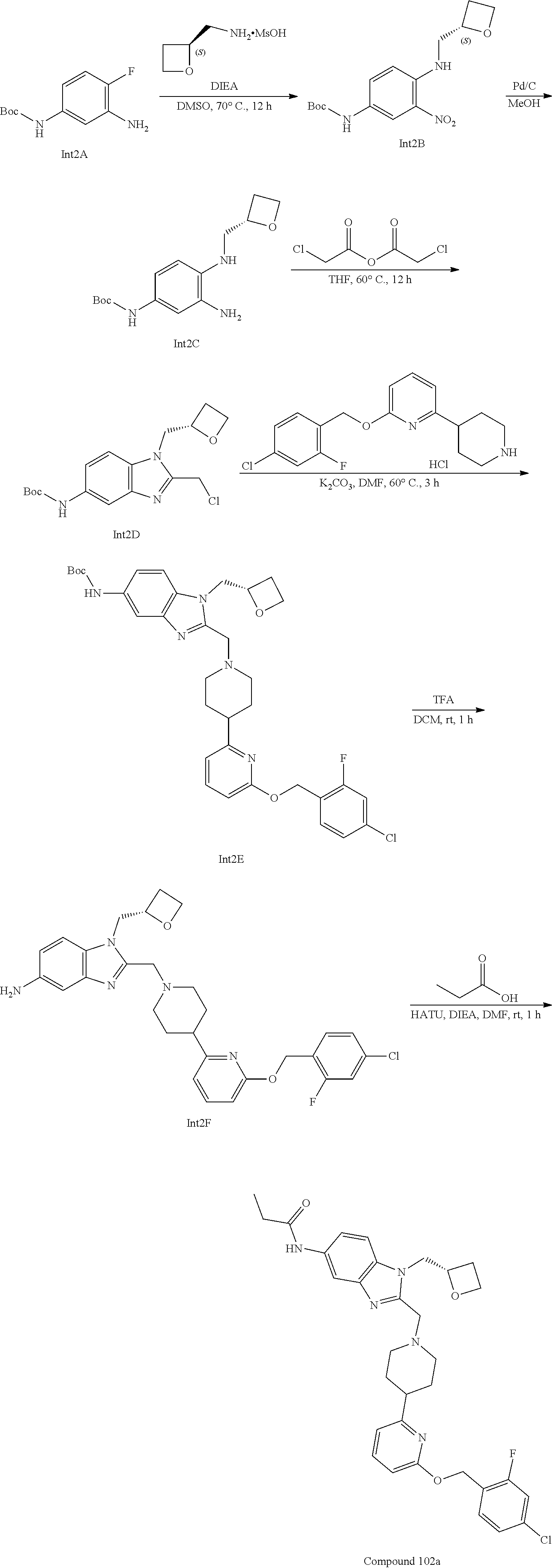

### Step A: tert-Butyl (S)-(3-nitro-4-((oxetan-2-ylmethyl)amino)phenyl) carbamate (Int2B)

To a solution of Int2A (2 g, 7.81 mmol) and (S)-oxetan-2-ylmethanamine MsOH salt (1.7 g, 9.37 mmol) in DMSO (20 mL) is added DIEA (3 g, 23.4 mmol) at room temperature. The reaction is stirred at 70° C. for 12 hours. After the reaction is complete, the reaction is quenched with water (50 mL) and extracted with ethyl acetate (100 mL×3). The organic layer is combined and washed with brine (100 mL×2), dried over sodium sulfate, filtered and concentrated in vacuo. The residue is purified by column chromatography to give Int2B (1.7 g, 68.0% yield) as red oil. MS Calcd.: 323.15; MS Found: 324.0 [M+H]⁺.

### Step B: tert-butyl (S)-(3-amino-4-((oxetan-2-ylmethyl)amino) phenyl) carbamate (Int2C)

A mixture of Int2B (1.7 g, 5.2 mmol) and Pd/C (170 mg, 10% in oil) in MeOH (20 mL) is stirred at room temperature under H₂ atmosphere for 2 hours. The mixture is filtered and concentrated under reduced pressure to give Int2C (1.4 g, yield: 93%).

### Step C: tert-Butyl (S)-(2-(chloromethyl)-1-(oxetan-2-ylmethyl)-1H-benzo [d]imidazol-5-yl)carbamate (Int2D)

A mixture of Int2C (1.4 g, 4.78 mmol) and 2-chloroacetic anhydride (893.5 mg, 5.25 mmol) in THE (15 mL) is stirred at 60° C. for 12 hours. The mixture is concentrated under reduced pressure and the residue is purified by column chromatography to give Int2D (1.4 g, yield: 83.3%). MS Calcd.: 351.1; MS Found: 352.2 [M+H]⁺.

### Step D: tert-butyl (S)-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)carbamate (Int2E)

A mixture of Int2D (1.2 g, 3.41 mmol), 2-((4-chloro-2-fluorobenzyl)oxy)-6-(piperidin-4-yl)pyridine HCl salt (1.33 g, 3.75 mmol) and K₂CO₃ (1.6 g, 11.94 mmol) in DMF (15 mL) is stirred at 60° C. for 3 hours. The mixture is diluted with Ethyl acetate (100 mL) and washed with water and brine. The organic layer is separated, dried over sodium sulfate, filtered and concentrated in vacuo. The residue is purified by column chromatography to give Int2E (1.0 g, yield: 46.3%). MS Calcd.: 635.27; MS Found: 636.3 [M+H]⁺.

### Step E: (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (Int2F)

To a solution of Int2E (1 g, 1.57 mmol) in DCM (10 mL) is added TFA (4 mL). The resulting mixture is stirred at room temperature for 1 hours. After the reaction is complete, the mixture is concentrated under vacuum, the mixture is added NaHCO₃ (aq) and extracted with DCM/MeOH (2×30 mL), The organic layer is separated, dried over sodium sulfate, filtered and concentrated under vacuum to Int2F (827 mg, crude). MS Calcd.: 535.22; MS Found: 536.3 [M+H]⁺.

### Step F: (S)—N-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)propionamide (Compound 102a)

To a solution of propionic acid (8.2 mg, 0.11 mmol) in DMF (1.5 mL) is added DIEA (35 mg, 0.27 mmol), HATU (44 mg, 0.117 mmol) and Int2F (50 mg, crude). The resulting mixture is stirred at room temperature for 1 h. After the reaction is complete, the residue is purified by Prep-HPLC to give Compound 102a (25.25 mg, yield: 46%) as white solid. MS Calcd.: 591.24; MS Found: 592.4 [M+H]⁺.

¹H NMR (400 MHz, DMSO-d₆) δ 9.78 (s, 1H), 7.91 (d, J=1.6 Hz, 1H), 7.63 (t, J=7.2 Hz, 1H), 7.56 (t, J=8.4 Hz, 1H), 7.49-7.51 (m, 1H), 7.46 (dd, J=9.6 Hz, 2.0 Hz, 1H), 7.28-7.35 (m, 2H), 6.87 (d, J=7.2 Hz, 1H), 6.67 (d, J=8.0 Hz, 1H), 5.38 (s, 2H), 5.06-5.12 (m, 1H), 4.64-4.70 (m, 1H), 4.53-4.57 (m, 1H), 4.44-4.49 (m, 1H), 4.35-4.40 (m, 1H), 3.87 (d, J=13.6 Hz, 1H), 3.73 (d, J=13.2 Hz, 1H), 2.96-2.99 (m, 1H), 2.83-2.87 (m, 1H), 2.59-2.72 (m, 2H), 2.38-2.44 (m, 1H), 2.29-2.35 (m, 2H), 2.12-2.24 (m, 2H), 1.66-1.83 (m, 4H), 1.10 (t, J=7.6 Hz, 3H).

### Example 3: (S)—N-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)cyclopropanecarboxamide (Compound 103a)

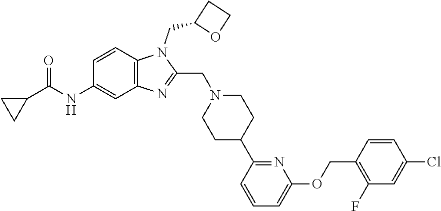 [SMILES: O=C(Nc1ccc2c(c1)nc(CN1CCC(c3cccc(OCc4ccc(Cl)cc4F)n3)CC1)n2C[C@@H]1CCO1)C1CC1]

The synthesis of Compound 103a is similar to that of Compound 102a except cyclopropanecarboxylic acid is used instead of propionic acid. Compound 103a (31.98 mg, yield: 57%) is finally obtained as a white solid. MS Calcd.: 603.24; MS Found: 604.2 [M+H]⁺. ¹H NMR (400 MHz, DMSO-d₆) δ 10.11 (s, 1H), 7.89 (d, J=1.6 Hz, 1H), 7.63 (t, J=8.0 Hz, 1H), 7.56 (t, J=8.0 Hz, 1H), 7.51 (d, J=8.8 Hz, 1H), 7.46 (dd, J=10.4 Hz, 2.4 Hz, 1H), 7.35 (dd, J=8.4 Hz, 1.6 Hz, 1H), 7.30 (dd, J=8.0 Hz, 1.6 Hz, 1H), 6.87 (d, J=7.2 Hz, 1H), 6.67 (d, J=8.4 Hz, 1H), 5.38 (s, 2H), 5.06-5.13 (m, 1H), 4.65-4.70 (m, 1H), 4.53-4.57 (m, 1H), 4.44-4.49 (m, 1H), 4.35-4.40 (m, 1H), 3.87 (d, J=13.2 Hz, 1H), 3.72 (d, J=13.2 Hz, 1H), 2.96-2.99 (m, 1H), 2.84-2.86 (m, 1H), 2.55-2.71 (m, 2H), 2.38-2.49 (m, 1H), 2.11-2.23 (m, 2H), 1.66-1.81 (m, 5H), 0.74-0.83 (m, 4H).

### Example 4: (S)—N-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)oxetane-3-carboxamide (Compound 104a)

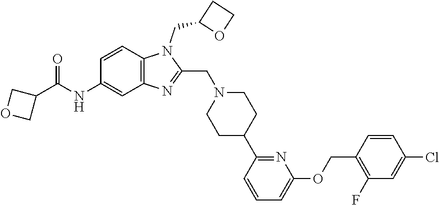 [SMILES: O=C(Nc1ccc2c(c1)nc(CN1CCC(c3cccc(OCc4ccc(Cl)cc4F)n3)CC1)n2C[C@@H]1CCO1)C1COC1]

The synthesis of Compound 104a is similar to that of Compound 102a except oxetane-3-carboxylic acid is used instead of propionic acid. Compound 104a (21.3 mg, yield: 36.8%) is finally obtained as a white solid. MS Calcd.: 619.24; MS Found: 620.5 [M+H]⁺. ¹H NMR (400 MHz, DMSO-d₆) δ 9.87 (s, 1H), 7.93 (d, J=1.6 Hz, 1H), 7.63 (t, J=8.0 Hz, 1H), 7.52-7.58 (m, 2H), 7.45 (dd, J=10.0 Hz, 2.0 Hz, 1H), 7.35 (dd, J=8.8 Hz, 2.0 Hz, 1H), 7.29 (dd, J=8.4 Hz, 2.0 Hz, 1H), 6.87 (d, J=7.2 Hz, 1H), 6.67 (d, J=8.4 Hz, 1H), 5.38 (s, 2H), 5.07-5.12 (m, 1H), 4.65-4.72 (m, 5H), 4.54-4.58 (m, 1H), 4.44-4.49 (m, 1H), 4.35-4.40 (m, 1H), 3.92-4.00 (m, 1H), 3.87 (d, J=13.6 Hz, 1H), 3.73 (d, J=13.2 Hz, 1H), 2.96-2.99 (m, 1H), 2.84-2.87 (m, 1H), 2.57-2.72 (m, 2H), 2.38-2.44 (m, 1H), 2.12-2.24 (m, 2H), 1.63-1.82 (m, 4H).

### Example 5: (S)—N-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)isobutyramide (Compound 105a)

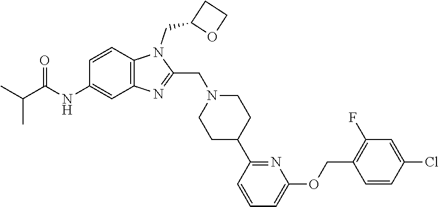 [SMILES: CC(C)C(=O)Nc1ccc2c(c1)nc(CN1CCC(c3cccc(OCc4ccc(Cl)cc4F)n3)CC1)n2C[C@@H]1CCO1]

The synthesis of Compound 105a is similar to that of Compound 102a except isobutyric acid is used instead of propionic acid. Compound 105a (21.5 mg, yield: 38%) is finally obtained as a white solid. MS Calcd.: 605.26; MS Found: 606.5 [M+H]⁺. ¹H NMR (400 MHz, DMSO-d₆) δ 9.74 (s, 1H), 7.92 (d, J=1.2 Hz, 1H), 7.63 (t, J=7.6 Hz, 1H), 7.56 (t, J=8.0 Hz, 1H), 7.51 (d, J=8.8 Hz, 1H), 7.46 (dd, J=10.0 Hz, 2.0 Hz, 1H), 7.36 (dd, J=8.8 Hz, 2.0 Hz, 1H), 7.29 (dd, J=8.4 Hz, 1.6 Hz, 1H), 6.87 (d, J=7.2 Hz, 1H), 6.67 (d, J=8.0 Hz, 1H), 5.38 (s, 2H), 5.06-5.12 (m, 1H), 4.64-4.70 (m, 1H), 4.53-4.58 (m, 1H), 4.44-4.49 (m, 1H), 4.34-4.39 (m, 1H), 3.87 (d, J=13.6 Hz, 1H), 3.73 (d, J=13.6 Hz, 1H), 2.96-3.00 (m, 1H), 2.84-2.87 (m, 1H), 2.56-2.71 (m, 3H), 2.37-2.46 (m, 1H), 2.12-2.24 (m, 2H), 1.66-1.83 (m, 4H), 1.11 (d, J=6.8 Hz, 6H).

### Example 6: (S)—N-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)-2-cyclopropylacetamide (Compound 106a)

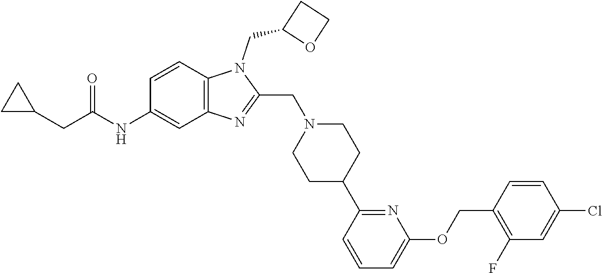 [SMILES: O=C(CC1CC1)Nc1ccc2c(c1)nc(CN1CCC(c3cccc(OCc4ccc(Cl)cc4F)n3)CC1)n2C[C@@H]1CCO1]

The synthesis of Compound 106a is similar to that of Compound 102a except 2-cyclopropylacetic acid is used instead of propionic acid. Compound 106a (26.75 mg, yield: 47%) is finally obtained as a white solid. MS Calcd.: 617.26; MS Found: 618.5 [M+H]⁺. ¹H NMR (400 MHz, DMSO-d₆) δ 9.73 (s, 1H), 7.92 (d, J=1.2 Hz, 1H), 7.63 (t, J=7.6 Hz, 1H), 7.56 (t, J=8.0 Hz, 1H), 7.51 (d, J=8.4 Hz, 1H), 7.46 (dd, J=10.0 Hz, 2.0 Hz, 1H), 7.35 (dd, J=8.4 Hz, 1.6 Hz, 1H), 7.30 (dd, J=8.0 Hz, 2.0 Hz, 1H), 6.87 (d, J=7.2 Hz, 1H), 6.67 (d, J=8.4 Hz, 1H), 5.38 (s, 2H), 5.07-5.13 (m, 1H), 4.65-4.70 (m, 1H), 4.53-4.58 (m, 1H), 4.44-4.49 (m, 1H), 4.35-4.40 (m, 1H), 3.87 (d, J=13.6 Hz, 1H), 3.73 (d, J=13.6 Hz, 1H), 2.96-2.99 (m, 1H), 2.84-2.87 (m, 1H), 2.57-2.71 (m, 2H), 2.38-2.45 (m, 1H), 2.12-2.24 (m, 4H), 1.66-1.81 (m, 4H), 1.04-1.12 (m, 1H), 0.46-0.51 (m, 2H), 0.18-0.22 (m, 2H).

### Example 7: (S)—N-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)-3-methoxypropanamide (Compound 107a)

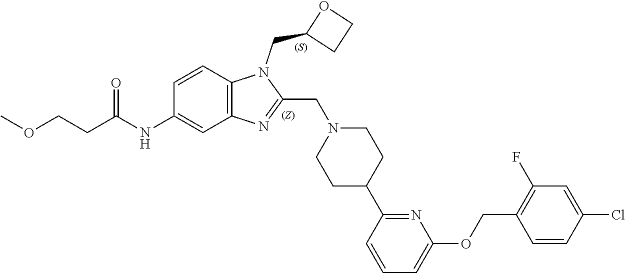

The synthesis of Compound 107a is similar to that of Compound 102a except 3-methoxypropanoic acid is used instead of propionic acid. Compound 107a (10.17 mg, yield: 17.5%) is finally obtained as a white solid. MS Calcd.: 621.2; MS Found: 622.1 [M+H]⁺. ¹H NMR (400 MHz, DMSO-d₆) 9.87 (s, 1H), 7.92 (d, J=1.6 Hz, 1H), 7.63 (t, J=8.0 Hz, 1H), 7.51-7.58 (m, 2H), 7.46 (dd, J=10.0 Hz, 2.0 Hz, 1H), 7.34 (dd, J=8.8 Hz, 1.6 Hz, 1H), 7.30 (dd, J=8.0 Hz, 1.6 Hz, 1H), 6.87 (d, J=7.2 Hz, 1H), 6.67 (d, J=8.0 Hz, 1H), 5.38 (s, 2H), 5.08-5.11 (m, 1H), 4.65-4.70 (m, 1H), 4.53-4.58 (m, 1H), 4.44-4.49 (m, 1H), 4.35-4.40 (m, 1H), 3.87 (d, J=13.6 Hz, 1H), 3.73 (d, J=13.6 Hz, 1H), 3.63 (t, J=6.0 Hz, 2H), 3.25 (s, 3H), 2.98 (d, J=10.8 Hz, 1H), 2.85 (d, J=10.8 Hz, 1H), 2.58-2.70 (m, 2H), 2.55 (t, J=6.0 Hz, 2H), 2.40-2.49 (m, 1H), 2.12-2.24 (m, 2H), 1.66-1.82 (m, 4H).

### Example 8: (S)—N-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)methanesulfonamide (Compound 108a)

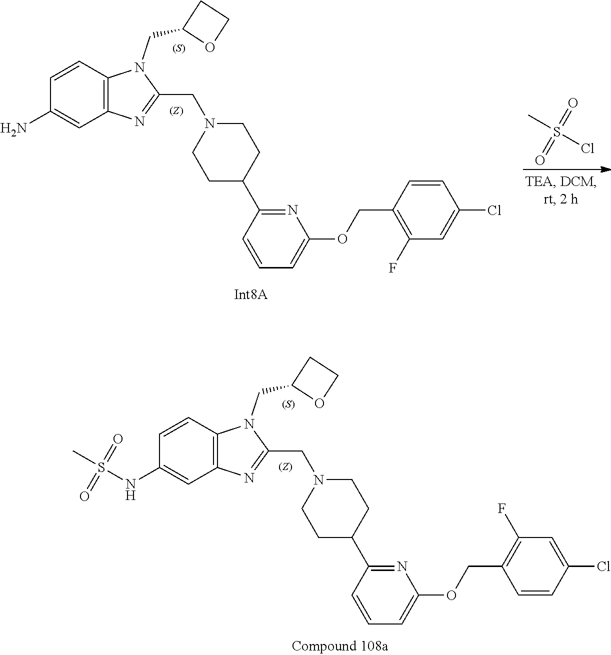

To a solution of Int8A (50 mg, 0.09 mmol) in DCM (2 mL) is added triethylamine (14 mg, 0.140 mmol) and methanesulfonyl chloride (12.8 mg, 0.110 mmol). The resulting mixture is stirred at room temperature for 2 hours. The solvent is removed in vacuo. The residue is purified by Prep-HPLC directly to give Compound 108a (16.41 mg, yield: 28.6%) as white solid. MS Calcd.: 613.19; MS Found: 614.4 [M+H]⁺.

¹H NMR (400 MHz, DMSO-d₆): δ 9.44 (s, 1H), 7.54-7.65 (m, 3H), 7.44-7.47 (m, 2H), 7.29 (dd, J=8.0 Hz, 2.0 Hz, 1H), 7.11 (dd, J=8.8 Hz, 2.0 Hz, 1H), 6.87 (d, J=7.2 Hz, 1H), 6.67 (d, J=8.0 Hz, 1H), 5.38 (s, 2H), 5.09-5.11 (m, 1H), 4.68-4.73 (m, 1H), 4.55-4.60 (m, 1H), 4.37-4.50 (m, 2H), 3.90 (d, J=13.6 Hz, 1H), 3.73 (d, J=13.6 Hz, 1H), 2.98 (d, J=11.2 Hz, 1H), 2.90 (s, 3H), 2.85 (d, J=11.6 Hz, 1H), 2.50-2.71 (m, 2H), 2.41-2.48 (m, 1H), 2.12-2.25 (m, 2H), 1.65-1.82 (m, 4H).

### Example 9: (S)-2-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)oxy)acetic acid (Compound 109a)

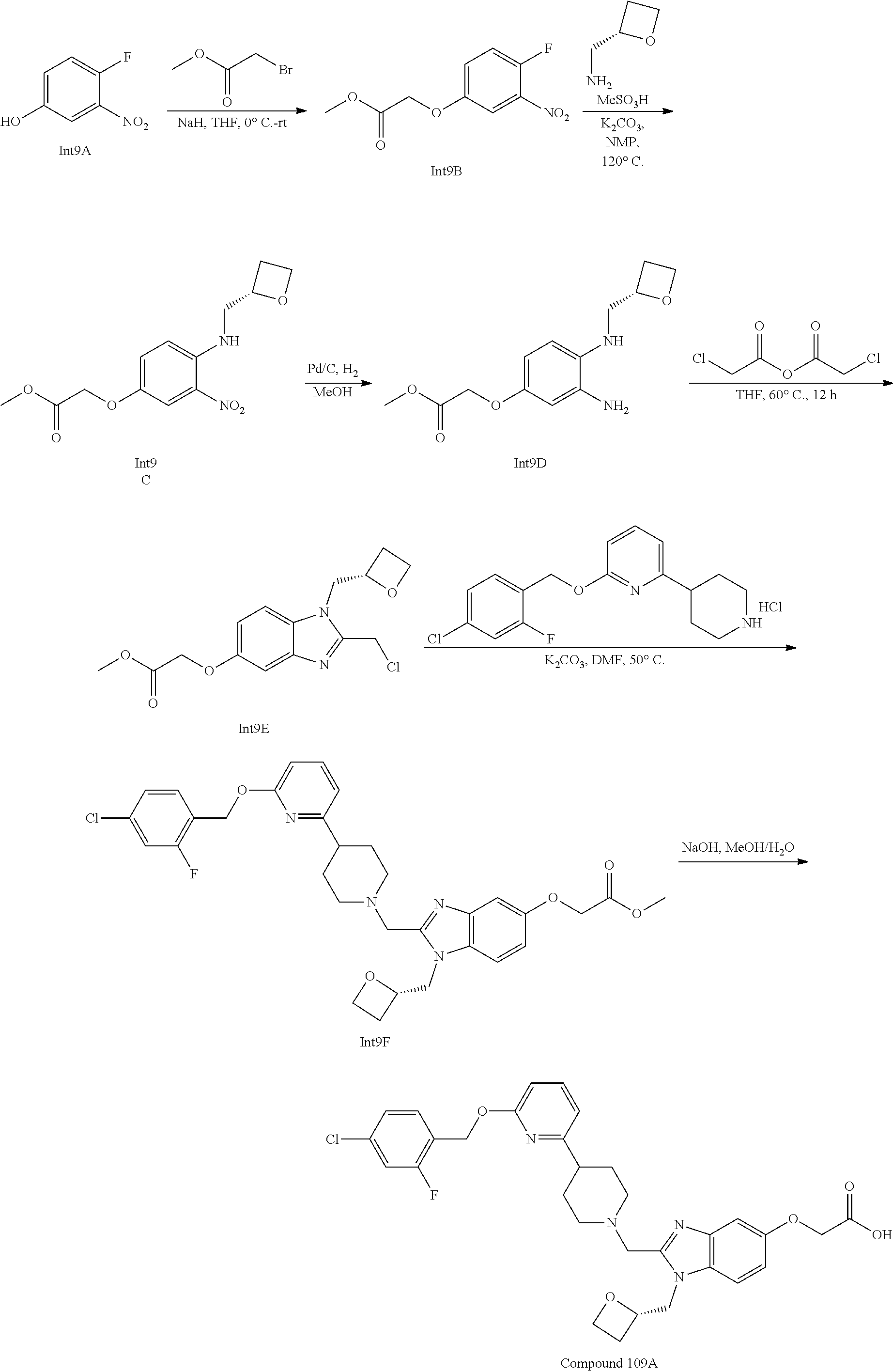

### Step A: methyl 2-(4-fluoro-3-nitrophenoxy)acetate (Int9B)

To a mixture of Int9A (500 mg, 3.2 mmol), methyl 2-bromoacetate (1.4 g, 9.6 mmol) in THE (20 mL) is added NaH (245 mg, 6.4 mmol, 60% purity) at 0° C., then the mixture is stirred at room temperature for 3 hours. After the reaction is complete, the mixture is quenched with Sat. NH₄Cl (10 mL), and extracted with Ethyl acetate. The organic layer is washed with brine (15 ml×2), dried over sodium sulfate, filtered, and concentrated in vacuo. The residue is purified by column chromatography to give Int9B (500 mg, yield: 68%).

### Step B: methyl (S)-2-(3-nitro-4-((oxetan-2-ylmethyl)amino) phenoxy)acetate (Int9C)

A mixture of Int9B (500 mg, 2.18 mmol), K₂CO₃ (904 mg, 6.54 mmol) and (S)-oxetan-2-ylmethanamine methanesulfonate (400 mg, 2.18 mmol) in NMP (10 mL) is stirred at 120° C. for 3 hours. The mixture is diluted with Ethyl acetate (20 mL). The organic layer is separated and washed with water and brine, dried over sodium sulfate, filtered, and concentrated in vacuo. The residue is purified by column chromatography to give methyl Int9C (270 mg, yield: 42%). MS Calcd.: 296.1; MS Found: 297.1 [M+H]⁺.

### Step C: Methyl (S)-2-(3-amino-4-((oxetan-2-ylmethyl)amino) phenoxy)acetate (Int9D)

A mixture of Int9C (270 mg, 0.91 mmol), and wet Pd/C (20 mg, 50 wt %) in MeOH (5 mL) is stirred at room temperature under H₂ atmosphere for 2 hours. The mixture is filtered and concentrated under reduced pressure to give Int9D (200 mg, crude).

### Step D: methyl (S)-2-((2-(chloromethyl)-1-(oxetan-2-ylmethyl)-1H-benzo[d]imidazol-5-yl)oxy)acetate (Int9E)

A mixture of Int9D (200 mg, crude) and 2-chloroacetic anhydride (128 mg, 0.75 mmol) in THE (5 mL) is stirred at 60° C. for 12 hours. The mixture is concentrated under reduced pressure and the residue is purified by column chromatography to give Int9E (210 mg, yield: 86%). MS Calcd.: 324.1; MS Found: 325.1 [M+H]⁺.

### Step E: methyl (S)-2-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)oxy)acetate (Int9F)

A mixture of Int9E (210 mg, 0.65 mmol), 2-((4-chloro-2-fluorobenzyl)oxy)-6-(piperidin-4-yl)pyridine hydrochloride (231 mg, 0.65 mmol) and K₂CO₃ (269 mg, 1.95 mmol) in DMF (5 mL) is stirred at 50° C. for 12 hours. The mixture is diluted with ethyl acetate (10 mL) and washed with water and brine. The organic layer is separated, dried over sodium sulfate, filtered and concentrated in vacuo. The residue is purified by column chromatography to give Int9F (10 mg, yield: 3%). MS Calcd.: 608.2; MS Found: 609.2 [M+H]⁺.

### Step F: (S)-2-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)oxy)acetic acid (Compound 109a)

A mixture of methyl Int9F (10 mg, 0.02 mmol) and NaOH (0.8 mg, 0.02 mmol) in MeOH/H₂O (5 mL/1 mL) is stirred at room temperature for 2 hours. The reaction mixture is purified by Prep-HPLC directly to Compound 109a (2.01 mg, yield: 17%) as a white solid. MS Calcd.: 594.2; MS Found: 595.1 [M+H]⁺.

¹H NMR (400 MHz, DMSO-d₆) δ7.54-7.64 (m, 2H), 7.44-7.50 (m, 2H), 7.28-7.30 (m, 1H), 7.02 (s, 1H), 6.84-6.86 (m, 2H), 6.66-6.68 (m, 1H), 5.37 (s, 2H), 5.07-5.09 (m, 1H), 4.63-4.68 (m, 1H), 4.52-4.57 (m, 3H), 4.42-4.48 (m, 1H), 4.36-4.38 (m, 1H), 3.86 (d, J=12.8 Hz, 1H), 3.72 (d, J=12.8 Hz, 1H), 2.96-2.98 (m, 1H), 2.84-2.86 (m, 1H), 2.56-2.68 (m, 2H), 2.38-2.44 (m, 1H), 2.10-2.22 (m, 2H), 1.62-1.81 (m, 4H).

### Example 10: (S)-2-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)acetic acid (Compound 110a)

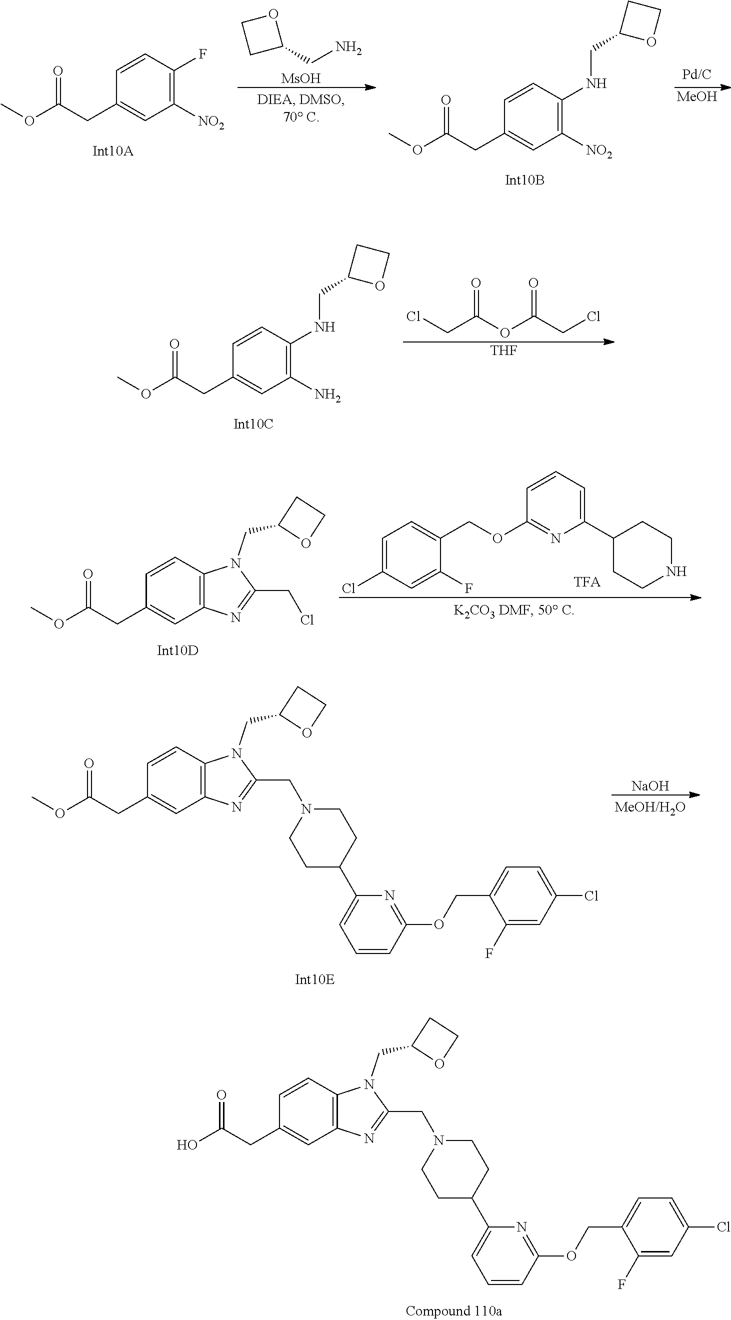

### Step A: Methyl (S)-2-(3-nitro-4-((oxetan-2-ylmethyl)amino)phenyl)acetate (Int10B)

To a solution of Int10A 450 mg, 2.11 mmol) and (S)-oxetan-2-ylmethanamine MsOH salt (463.9 mg, 2.53 mmol) in DMSO (10 mL) is added DIEA (817 mg, 6.33 mmol) at room temperature. The reaction is stirred at 70° C. for 3 hours. After the reaction is complete, the reaction is quenched with water (50 mL) and extracted with ethyl acetate (100 mL×3). The organic layer is combined and washed with brine (100 mL×2), dried over sodium sulfate, filtered and concentrated in vacuo. The residue is purified by column chromatography to give Int10B (610 mg, crude) as a grey solid. MS Calcd.: 280.11; MS Found: 281.0 [M+H]⁺.

### Step B: Methyl (S)-2-(3-amino-4-((oxetan-2-ylmethyl)amino)phenyl)acetate (Int10C)

To a solution of Int10B (610 mg, crude) in MeOH (6 mL) is added wet Pd/C (60 mg, 50 wt %). The reaction vessel is purged with H₂ three times and the reaction is stirred at room temperature under H₂ (15 psi) for 1 hour. After the reaction is complete, the reaction mixture is filtered and concentrated under reduced pressure to afford Int10C (3.6 g, crude) as colorless oil. MS Calcd.: 250.13; MS Found: 251.0 [M+H]⁺.

### Step C: Methyl (S)-2-(2-(chloromethyl)-1-(oxetan-2-ylmethyl)-1H-benzo[d]imidazol-5-yl)acetate (Int10D)

To a solution of Int10C (360 mg, crude) in THE (5 mL) is added 2-chloroacetic anhydride (270 mg, 1.59 mmol). The reaction is stirred at room temperature for 1 hour, then stirred at 60° C. for 16 hours. After the reaction is complete, the reaction is filtered and concentrated to give crude product. The crude product is purified by column chromatography to give Int10D (370 mg, 83% yield) as a grey solid. MS Calcd.: 308.09; MS Found: 309.1 [M+H]⁺.

### Step D: Methyl (S)-2-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)acetate (Int10E)

To a solution of Int10D (70 mg, 0.23 mmol) and 2-((4-chloro-2-fluorobenzyl)oxy)-6-(piperidin-4-yl)pyridine (81 mg, 0.23 mmol, HCl salt) in DMF (1 mL) is added K₂CO₃ (94 mg, 0.69 mmol). The reaction is stirred at 50° C. for 2 hours. After the reaction is complete, the reaction is quenched with water (20 mL) and extracted with ethyl acetate (30 mL×3). The organic layer is combined and washed with brine (50 mL×2), dried over sodium sulfate, filtered and concentrated in vacuo to give Int10E (100 mg, crude) as yellow oil. MS Calcd.: 592.23; MS Found: 593.0 [M+H]⁺.

### Step E: (S)-2-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)acetic acid (Compound 110a)

To a solution of Int10E (100 mg, crude) in MeOH (3 mL)/H₂O (1 mL) is added NaOH (20.3 mg, 0.51 mmol). The reaction is stirred at room temperature for 1 hours. After the reaction is complete, the reaction mixture is filtered and purified by Prep-HPLC to give Compound 110a (39.15 mg, 40% yield) as a white solid. MS Calcd.: 578.21; MS Found: 579.4 [M+H]⁺.

¹H NMR (400 MHz, DMSO-d₆): δ 7.62 (t, J=8.0 Hz, 1H), 7.52-7.58 (m, 2H), 7.45-7.47 (m, 2H), 7.28-7.31 (m, 1H), 7.10 (dd, J=8.4 Hz, J=0.8 Hz, 1H), 6.87 (d, J=7.2 Hz, 1H), 6.67 (d, J=8.0 Hz, 1H), 5.37 (s, 2H), 5.07-5.13 (m, 1H), 4.67-4.72 (m, 1H), 4.55-4.60 (m, 1H), 4.44-4.49 (m, 1H), 4.35-4.40 (m, 1H), 3.89 (d, J=13.2 Hz, 1H), 3.75 (d, J=13.2 Hz, 1H), 3.62 (s, 2H), 2.96-2.99 (m, 1H), 2.84-2.87 (m, 1H), 2.57-2.72 (m, 2H), 2.39-2.47 (m, 1H), 2.12-2.24 (m, 2H), 1.66-1.81 (m, 4H).

### Example 11: (S)-2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazole-5-carboxylic acid (Compound 111a)

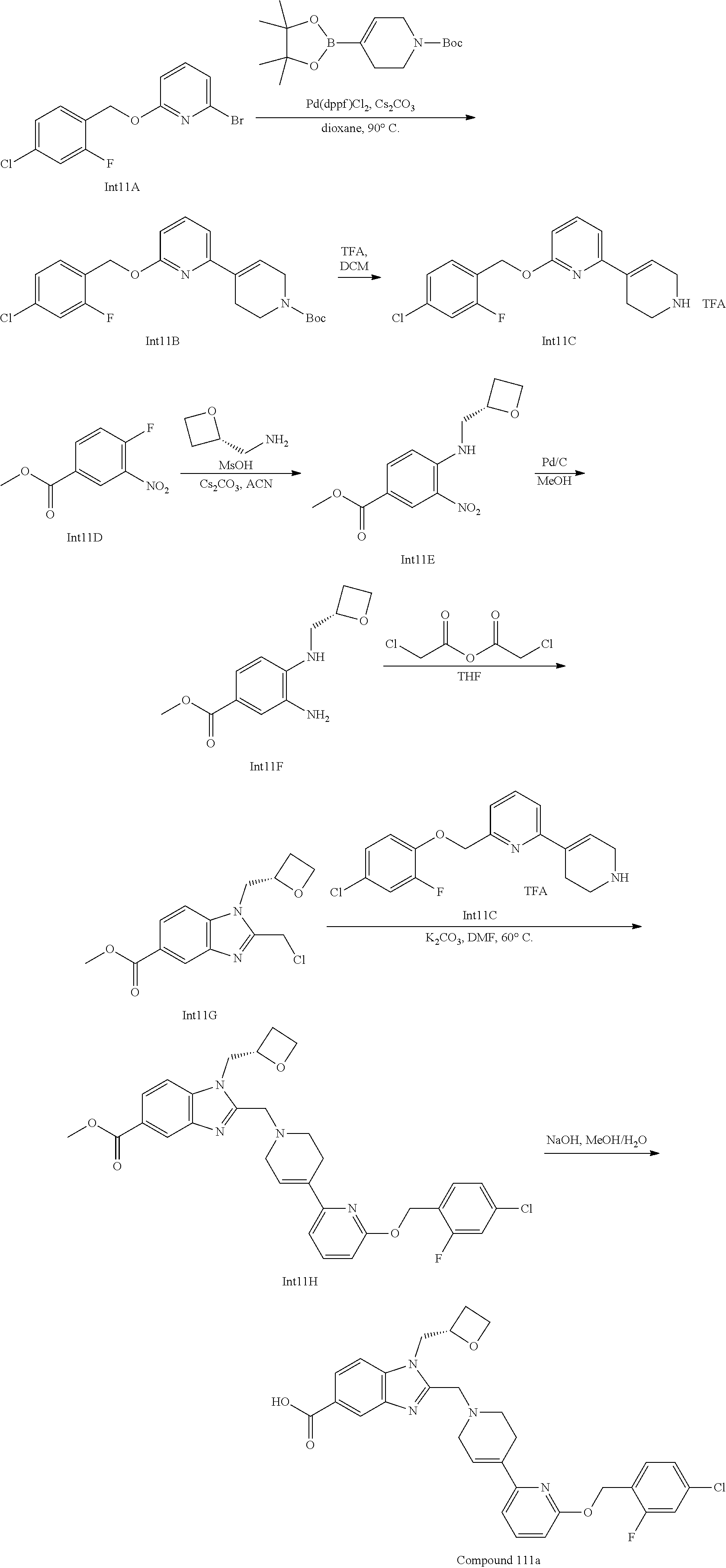

### Step A: tert-Butyl 6-((4-chloro-2-fluorobenzyl)oxy)-3′,6′-dihydro-[2,4′-bipyridine]-1′(2′H)-carboxylate (Int11B)

A mixture of Int11A (1 g, 3.2 mmol), tert-butyl 4-(4,4,5,5-tetramethyl-1,3,2-dioxaborolan-2-yl)-3,6-dihydropyridine-1(2H)-carboxylate (1.17 g, 3.8 mmol), Pd(dppf)Cl₂ (258 mg, 0.32 mmol) and Cs₂CO₃ (3.08 g, 9.6 mmol) in dioxane (10 mL) and H₂O (1 mL) is purged with N₂ for three times. The mixture is stirred at 90° C. under N₂ for 3 hours. After the reaction is complete, the mixture is quenched with water (50 mL) and extracted with ethyl acetate (100 mL×3). The organic layers are combined and washed with brine (100 mL×2), dried over sodium sulfate, filtered, and concentrated in vacuo. The residue is purified by column chromatography to give Int11B (1.44 g, crude) as yellow oil. MS Calcd.: 418.15; MS Found: 419.0 [M+1]⁺.

### Step B: 6-((4-Chloro-2-fluorobenzyl)oxy)-1′,2′,3′,6′-tetrahydro-2,4′-bipyridine TFA salt (Int11C)

To a solution of Int11B (740 mg, crude) in DCM (10 mL) is added TFA (5 mL). The mixture is stirred at room temperature for 1 hour. After the reaction is complete, the reaction is concentrated to give Int11C (800 mg, crude) as yellow oil. MS Calcd.: 318.1; MS Found: 319.0 [M+H]⁺.

### Step C: Methyl (S)-3-nitro-4-((oxetan-2-ylmethyl)amino)benzoate (Int11E)

To a solution of Int11D (500 mg, 2.5 mmol) and (S)-oxetan-2-ylmethanamine MsOH salt (460 mg, 2.5 mmol) in ACN (10 mL) is added Cs₂CO₃ (2.45 g, 7.5 mmol) at room temperature. The reaction is stirred at 70° C. for 3 hours. After the reaction is complete, the reaction is quenched with water (50 mL) and extracted with ethyl acetate (100 mL×3). The organic layers are combined and washed with brine (100 mL×2), dried over sodium sulfate, filtered, and concentrated in vacuo. The residue is purified by column chromatography to give Int11E (500 mg, 72% yield) as an orange solid. MS Calcd.: 266.1; MS Found: 267.3 [M+H]⁺.

### Step D: Methyl (S)-3-amino-4-((oxetan-2-ylmethyl)amino)benzoate (Int11F)

To a solution of Int11E (100 mg, 0.14 mmol) in MeOH (2 mL) is added wet Pd/C (20 mg, 50 wt %). The reaction vessel is purged with H₂ for three times and the reaction is stirred at room temperature under H₂ (15 psi) for 1 hour. After the reaction is complete, the reaction is filtered and concentrated under reduced pressure to afford Int11F (100 mg, crude) as colorless oil. MS Calcd.: 236.1; MS Found: 237.0 [M+H]⁺.

### Step E: Methyl (S)-2-(chloromethyl)-1-(oxetan-2-ylmethyl)-1H-benzo[d]imidazole-5-carboxylate (Int11G)

To a solution of Int11F (100 mg, crude) in THE (3 mL) is added 2-chloroacetic anhydride (79.7 mg, 0.47 mmol). The reaction is stirred at room temperature for 1 hours, then stirred at 80° C. for 72 hours. After the reaction is complete, the reaction is concentrated and purified by column chromatography to Int11G (50 mg, 40% yield in two steps) as a grey solid. MS Calcd.: 294.1; MS Found: 295.1 [M+H]⁺.

### Step F: Methyl (S)-2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazole-5-carboxylate (Int11H)

To a solution of Int11G (50 mg, 0.17 mmol) and Int11C (86.7 mg, crude) in DMF (2 mL) is added K₂CO₃ (70 mg, 0.51 mmol). The reaction is stirred at 60° C. for 2 hours. After the reaction is complete, the reaction is quenched with water (20 mL) and extracted with ethyl acetate (30 mL×3). The organic layers are combined and washed with brine (50 mL×2), dried over sodium sulfate, filtered and concentrated in vacuo to give Int11H (50 mg, crude) as yellow oil. MS Calcd.: 576.2; MS Found: 577.2 [M+H]⁺.

### Step G: (S)-2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazole-5-carboxylic acid (Compound 11a)

To a solution of Int11H (50 mg, crude) in MeOH/H₂O (3 mL/1 mL) is added NaOH (10.4 mg, 0.26 mmol). The reaction is stirred at room temperature for 1 hours.

After the reaction is complete, the reaction is filtered and purified by Prep-HPLC to give Compound 111a (8.71 mg, 18% yield) as white solid. MS Calcd.: 562.18; MS Found: 563.2 [M+H]⁺.

¹H NMR (400 MHz, DMSO-d₆): δ 8.35 (s, 1H), 7.00-8.01 (m, 1H), 7.58-7.64 (m, 2H), 7.48 (t, J=8.0 Hz, 1H), 7.16-7.22 (m, 2H), 7.04 (d, J=7.6 Hz, 1H), 6.70-6.72 (m, 1H), 6.66 (d, J=8.0 Hz, 1H), 5.42 (s, 2H), 5.20-5.25 (m, 1H), 4.81-4.83 (m, 1H), 4.66-4.70 (m, 1H), 4.58-4.62 (m, 1H), 4.41-4.46 (m, 1H), 4.13 (d, J=14.0 Hz, 1H), 4.02 (d, J=13.6 Hz, 1H), 3.27-3.29 (m, 2H), 2.82-2.84 (m, 2H), 2.69-2.77 (m, 1H), 2.59-2.65 (m, 2H), 2.44-2.53 (m, 1H).

### Example 12: (S)-2-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)acetic acid (Compound 112a)

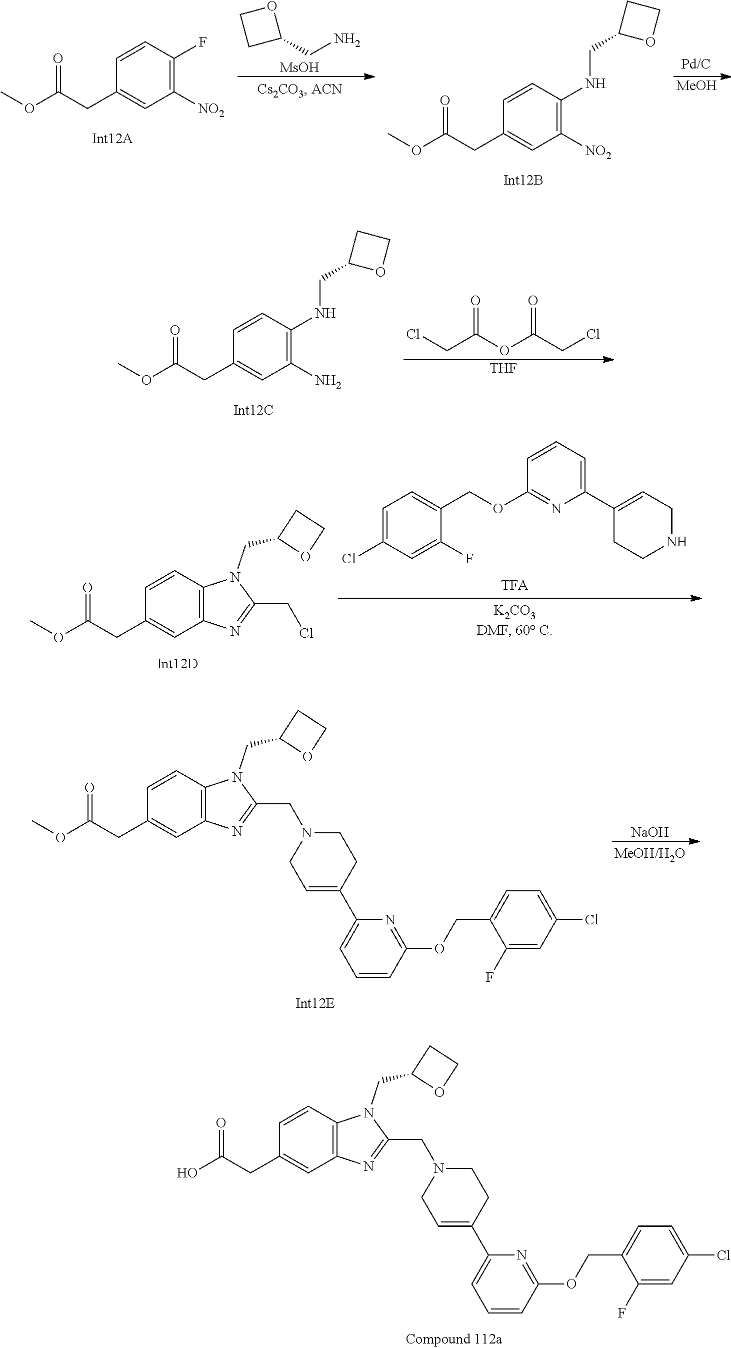

### Step A: Methyl (S)-2-(3-nitro-4-((oxetan-2-ylmethyl)amino)phenyl)acetate (Int12B)

To a solution of Int12A (500 mg, 2.35 mmol) and (S)-oxetan-2-ylmethanamine MsOH salt (429.8 mg, 2.35 mmol) in ACN (10 mL) is added Cs₂CO₃ (2.29 g, 7.05 mmol) at room temperature. The reaction is stirred at 70° C. for 3 hours. After the reaction is complete, the reaction is quenched with water (50 mL) and extracted with ethyl acetate (100 mL×3). The organic layer is combined and washed with brine (100 mL×2), dried over sodium sulfate, filtered and concentrated in vacuo. The residue is purified by column chromatography to give Int12B (100 mg, 15% yield) as a grey solid. MS Calcd.: 280.1; MS Found: 281.2 [M+H]⁺.

### Step B: Methyl (S)-2-(3-amino-4-((oxetan-2-ylmethyl)amino)phenyl)acetate (Int12C)

To a solution of Int12B (100 mg, 0.14 mmol) in MeOH (2 mL) is added wet Pd/C (20 mg, 50 wt %). The reaction vessel is purged with H₂ for three times and the reaction mixture is stirred at room temperature under H₂ (15 psi) for 1 hour. After the reaction is complete, the reaction mixture is filtered and concentrated under reduced pressure to afford Int12C (50 mg, crude) as colorless oil. MS Calcd.: 250.1; MS Found: 251.1 [M+H]⁺.

### Step C: Methyl (S)-2-(2-(chloromethyl)-1-(oxetan-2-ylmethyl)-1H-benzo[d]imidazol-5-yl)acetate (Int12D)

To a solution of Int12C (50 mg, crude) in THE (3 mL) is added 2-chloroacetic anhydride (34 mg, 0.20 mmol). The reaction is stirred at room temperature for 1 hour, then stirred at 60° C. for 16 hours. After the reaction is complete, the reaction is filtered and concentrated to give crude product. The crude product is purified by column chromatography to give Int12D (60 mg, crude) as a grey solid. MS Calcd.: 308.1; MS Found: 309.0 [M+H]⁺.

### Step D: methyl (S)-2-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)acetate (Int12E)

To a solution of Int12D and 6-((4-chloro-2-fluorobenzyl)oxy)-1′,2′,3′,6′-tetrahydro-2,4′-bipyridine, TFA salt (99.7 mg, crude) in DMF (2 mL) is added K₂CO₃ (80 mg, 0.59 mmol). The reaction is stirred at 60° C. for 2 hours. After the reaction is complete, the reaction is quenched with water (20 mL) and extracted with ethyl acetate (30 mL×3). The organic layer is combined and washed with brine (50 mL×2), dried over sodium sulfate, filtered and concentrated in vacuo to give Int12E (70 mg, crude) as yellow oil. MS Calcd.: 590.2; MS Found: 591.0 [M+H]⁺.

### Step E: (S)-2-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)acetic acid (Compound 112a)

To a solution of Int12E (70 mg, crude) in MeOH/H₂O (3 mL/1 mL) is added NaOH (14.2 mg, 0.36 mmol). The reaction is stirred at room temperature for 1 hours. After the reaction is complete, the reaction is filtered and purified by Prep-HPLC to give Compound 112a (10.33 mg, 9% yield in 4 steps) as a white solid. MS Calcd.: 576.19; MS Found: 577.0 [M+H]⁺.

¹H NMR (400 MHz, DMSO-d₆): δ 7.68 (t, J=8.0 Hz, 1H), 7.51-7.57 (m, 2H), 7.45-7.48 (m, 2H), 7.28-7.31 (m, 1H), 7.07-7.12 (m, 2H), 6.71-6.73 (m, 2H), 5.40 (s, 2H), 5.02-5.08 (m, 1H), 4.65-4.71 (m, 1H), 4.53-4.58 (m, 1H), 4.43-4.48 (m, 1H), 4.33-4.38 (m, 1H), 4.01 (d, J=13.2 Hz, 1H), 3.88 (d, J=13.2 Hz, 1H), 3.60 (s, 2H), 3.16-3.27 (m, 4H), 2.72 (t, J=6.0 Hz, 2H), 2.60-2.65 (m, 1H), 2.36-2.41 (m, 1H).

### Example 13: (S)-3-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)propanoic acid (Compound 113a)

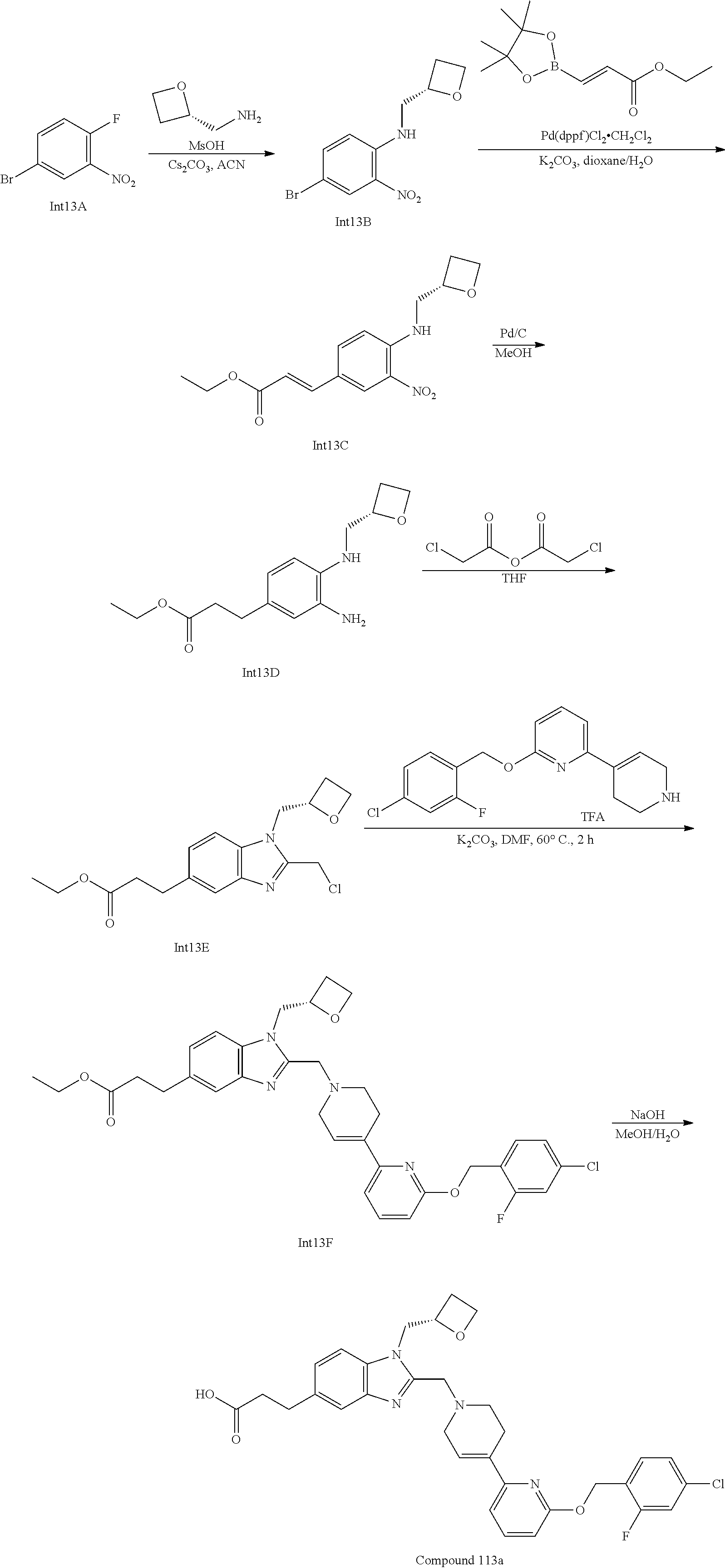

### Step A: (S)-4-Bromo-2-nitro-N-(oxetan-2-ylmethyl)aniline (Int13B)

To a solution of Int13A (500 mg, 2.28 mmol) and (S)-oxetan-2-ylmethanamine MsOH salt (416 mg, 2.28 mmol) in ACN (10 mL) is added Cs₂CO₃ (2.22 g, 6.85 mmol) at room temperature. The reaction is stirred at 70° C. for 3 hours. After the reaction is complete, the reaction is quenched with water (50 mL) and extracted with ethyl acetate (100 mL×3). The organic layer is combined and washed with brine (100 mL×2), dried over sodium sulfate, filtered and concentrated in vacuo. The residue is purified by column chromatography to give Int13B (550 mg, 84% yield) as an orange solid. MS Calcd.: 286.0; MS Found: 287.1 [M+H]⁺.

### Step B: Ethyl (S,E)-3-(3-nitro-4-((oxetan-2-ylmethyl)amino)phenyl)acrylate (Int13C)

To a mixture of Int13B (100 mg, 0.14 mmol) and ethyl (E)-3-(4,4,5,5-tetramethyl-1,3,2-dioxaborolan-2-yl)acrylate (94.8 mg, 0.17 mmol) in dioxane (1.6 mL)/H₂O (0.4 mL) is added Pd(dppf)Cl₂·CH₂Cl₂ (28.5 mg, 0.014 mmol) and K₂CO₃ (144.8 mg, 0.42 mmol). The reaction vessel is purged with N₂ for three times and the reaction is stirred at 90° C. under N₂ for 3 hours. After the reaction is complete, the reaction is quenched with water (50 mL) and extracted with ethyl acetate (100 mL×3).

The organic layer is combined and washed with brine (100 mL×2), dried over sodium sulfate, filtered and concentrated in vacuo. The residue is purified by column chromatography to afford Int13C (140 mg, crude) as yellow oil. MS Calcd.: 306.1; MS Found: 307.1 [M+H]⁺.

### Step C: Ethyl (S)-3-(3-amino-4-((oxetan-2-ylmethyl)amino)phenyl)propanoate (Int13D)

To a solution of Int13C (140 mg, crude) in MeOH (2 mL) is added wet Pd/C (20 mg, 50 wt %). The reaction vessel is purged with H₂ three times and the reaction is stirred at room temperature under H₂ (15 psi) for 1 hour. After the reaction is complete, the reaction is filtered and concentrated under reduced pressure to afford Int13D (100 mg, crude) as colorless oil. MS Calcd.: 278.2; MS Found: 279.0 [M+H]⁺.

### Step D: Ethyl (S)-3-(2-(chloromethyl)-1-(oxetan-2-ylmethyl)-1H-benzo[d]imidazol-5-yl)propanoate (Int13E)

To a solution of Int13D (100 mg, crude) in THE (3 mL) is added 2-chloroacetic anhydride (67.7 mg, 0.40 mmol). The reaction is stirred at room temperature for 1 hour, then stirred at 60° C. for 16 hours. After the reaction is complete, the reaction is filtered and concentrated to give crude product. The crude product is purified by column chromatography to give Int13E (100 mg, 83% yield in 3 steps) as a grey solid. MS Calcd.: 336.1; MS Found: 337.2 [M+H]⁺.

### Step E: Ethyl (S)-3-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)propanoate (Int13F)

To a solution of Int13E (100 mg, 0.30 mmol) and 6-((4-chloro-2-fluorobenzyl)oxy)-1′,2′,3′,6′-tetrahydro-2,4′-bipyridine, TFA salt (152 mg, crude) in DMF (2 mL) is added K₂CO₃ (123 mg, 0.90 mmol). The reaction is stirred at 60° C. for 2 hours. After the reaction is complete, the reaction is quenched with water (20 mL) and extracted with ethyl acetate (30 mL×3). The organic layer is combined and washed with brine (50 mL×2), dried over sodium sulfate, filtered and concentrated in vacuo to give Int13F (100 mg, crude) as yellow oil.

### Step F: (S)-3-(2-((6-((4-Chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)propanoic acid (Compound 113a)

To a solution of Int13F (100 mg, crude) in MeOH/H₂O (3 mL/1 mL) is added NaOH (19.4 mg, 0.49 mmol). The reaction is stirred at room temperature for 1 hour. After the reaction is complete, the reaction is filtered and the filtrate is purified by Prep-HPLC to give Compound 113a (7.49 mg, 4.2% yield in 2 steps) as a white solid. MS Calcd.: 590.21; MS Found: 591.3 [M+H]⁺.

¹H NMR (400 MHz, DMSO-d₆): δ 7.60 (t, J=8.0 Hz, 1H), 7.46-7.51 (m, 3H), 7.16-7.21 (m, 3H), 7.04 (d, J=7.6 Hz, 1H), 6.70-6.71 (m, 1H), 6.67 (d, J=8.4 Hz, 1H), 5.42 (s, 2H), 5.17-5.23 (m, 1H), 4.74-4.80 (m, 1H), 4.56-4.65 (m, 2H), 4.39-4.44 (m, 1H), 4.09 (d, J=13.6 Hz, 1H), 3.99 (d, J=13.6 Hz, 1H), 3.25-3.27 (m, 2H), 3.04 (t, J=7.6 Hz, 2H), 2.80-2.83 (m, 2H), 2.67-2.75 (m, 1H), 2.60-2.64 (m, 4H), 2.43-2.51 (m, 1H).

### Example 14: N-(2-((4-((R)-2-(4-chloro-2-fluorophenyl)-2,3-dihydrobenzo[b][1,4]dioxin-5-yl)piperidin-1-yl)methyl)-1-(((S)-oxetan-2-yl)methyl)-1H-benzo[d]imidazol-5-yl)acetamide (Compound 114a)

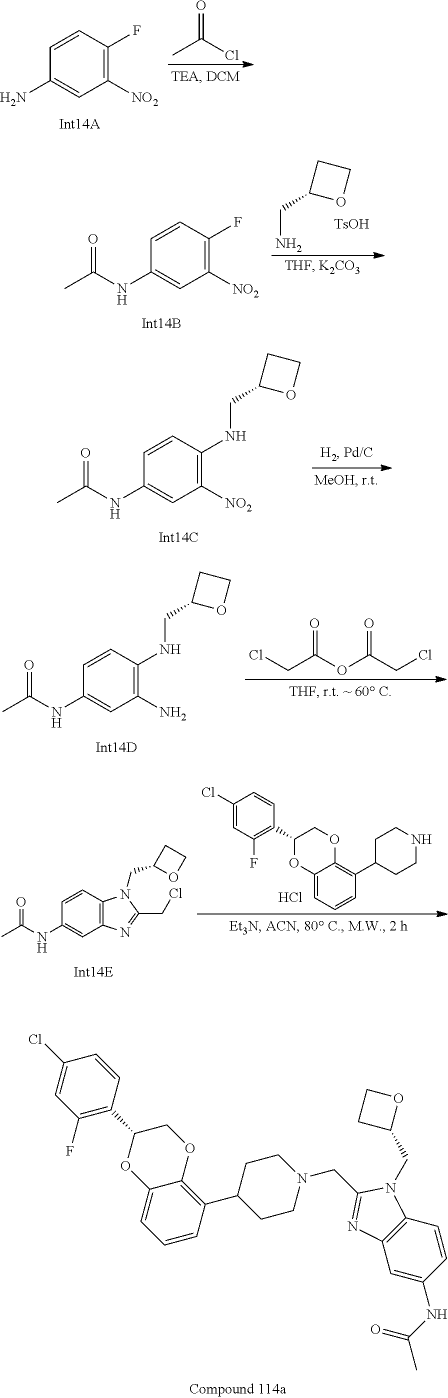

### Step A: N-(4-fluoro-3-nitrophenyl)acetamide (Int14B)

To a solution of Int14A (500 mg, 3.2 mmol) and TEA (971 mg, 9.60 mmol) in DCM (5 mL) is added acetyl chloride (375 mg, 4.80 mmol) at 0° C. The mixture is stirred at room temperature for 4 hours. The reaction mixture is poured into water (50 mL) and the mixture is extracted with ethyl acetate (2*60 mL). The combined organic layers is dried over Na₂SO₄, filtered and concentrated in vacuo to afford the title compound Int14B (650 mg, 99% yield). ¹HNMR (400 MHz, CDCl₃) δ 8.24 (d, J=6.0 Hz, 1H), 7.87 (d, J=9.2 Hz, 1H), 7.70 (br.s, 1H), 7.25 (t, J=9.6 Hz, 1H), 2.23 (s, 3H).

### Step B: (S)—N-(3-nitro-4-((oxetan-2-ylmethyl)amino)phenyl)acetamide (Int14C)

A mixture of Int14B (650 mg, 3.20 mmol), (S)-oxetan-2-ylmethanamine 4-methylbenzenesulfonate (1.02 g, 3.90 mmol) and K₂CO₃ (900 mg, 6.60 mmol) in THF (10 mL) is stirred at room temperature for 24 hours. The mixture is filtered. The filtrate is concentrated in vacuo and the residue is purified by SGC (PE/EA=2:1) to afford the title compound Int14C as an orange solid (240 mg, 28% yield). ¹HNMR (400 MHz, CDCl₃) δ 8.31 (s, 1H), 8.10 (d, J=2.4 Hz, 1H), 7.79 (dd, J=9.2, 2.4 Hz, 1H), 7.22 (s, 1H), 6.91 (d, J=9.2 Hz, 1H), 5.13 (ddd, J=11.6, 7.2, 4.4 Hz, 1H), 4.74 (dt, J=14.4, 7.2 Hz, 1H), 4.61 (dt, J=9.2, 6.0 Hz, 1H), 3.57 (s, 2H), 2.55-2.81 (m, 2H), 2.17 (s, 3H).

### Step C: (S)—N-(3-amino-4-((oxetan-2-ylmethyl)amino)phenyl)acetamide (Int14D)

A mixture of Int14C (240 mg, 0.910 mmol) and Pd/C (30.0 mg, 10% by weight) in MeOH (5 mL) is stirred under H₂ (1 atm) at room temperature for 16 hours. The reaction solution is filtered and the filtrate is concentrated in vacuo to afford the title compound Int14D (200 mg, 95% yield). ¹HNMR (400 MHz, CDCl₃) δ 7.12 (s, 1H), 7.09 (d, J=2.4 Hz, 1H), 6.70 (dd, J=8.4, 2.4 Hz, 1H), 6.60 (d, J=8.4 Hz, 1H), 5.06 (ddd, J=14.0, 6.8, 4.0 Hz, 1H), 4.73 (dt, J=14.4, 7.2 Hz, 1H), 4.59 (dt, J=9.2, 6.0 Hz, 1H), 3.31 (ddd, J=16.8, 12.8, 5.2 Hz, 2H), 2.53-2.76 (m, 2H), 2.12 (s, 3H).

### Step D: (S)—N-(2-(chloromethyl)-1-(oxetan-2-ylmethyl)-1H-benzo[d]imidazol-5-yl)acetamide (Int14E)

A suspension of Int14D (200 mg, 0.900 mmol) and 2-chloroacetic anhydride (160 mg, 0.940 mmol) in THE (6 mL) is stirred at 25° C. for 1 hour and then at 60° C. for 1.5 hours. The mixture is diluted with water (50 mL) and the mixture is extracted with ethyl acetate (4*60 mL). The extracts were dried over Na₂SO₄ and concentrated in vacuo to afford the title compound Int14E (240 mg, crude). MS Calcd.: 293.1; MS Found: 294.2 [M+H]⁺.

### Step E: N-(2-((4-((R)-2-(4-chloro-2-fluorophenyl)-2,3-dihydrobenzo[b][1,4]dioxin-5-yl)piperidin-1-yl)methyl)-1-((S)-oxetan-2-ylmethyl)-1H-benzo[d]imidazol-5-yl)acetamide (Compound 114a)

A solution of Int14E (80.0 mg, crude), (R)-4-(2-(4-chloro-2-fluorophenyl)-2,3-dihydrobenzo[b][1,4]dioxin-5-yl)piperidine hydrochloride (94.0 mg, 0.240 mmol) and TEA (55.0 mg, 0.540 mmol) in ACN (2.5 mL) is stirred under microwave irradiation at 80° C. for 2 hours. The mixture is filtered and the filtrate is purified by prep-HPLC (ACN/NH₄HCO₃, aq) to afford the title compound Compound 114a (28.0 mg, 20% yield). MS Calcd.: 604.2; MS Found: 605.2 [M+H]⁺.

¹HNMR (400 MHz, DMSO-d₆) δ 9.86 (s, 1H), 7.88 (d, J=1.6 Hz, 1H), 7.49-7.57 (m, 3H), 7.38 (dd, J=8.4, 1.6 Hz, 1H), 7.32 (dd, J=8.8, 1.6 Hz, 1H), 6.80-6.84 (m, 3H), 5.43 (dd, J=8.0, 2.0 Hz, 1H), 5.05-5.11 (m, 1H), 4.67 (dd, J=15.2, 6.8 Hz, 1H), 4.48-4.57 (m, 3H), 4.34-4.40 (m, 1H), 4.13 (dd, J=11.2, 8.0 Hz, 1H), 3.86 (d, J=13.2 Hz, 1H), 3.73 (d, J=13.2 Hz, 1H), 2.98 (d, J=11.2 Hz, 1H), 2.83-2.89 (m, 2H), 2.64-2.73 (m, 1H), 2.37-2.46 (m, 1H), 2.12-2.24 (m, 2H), 2.04 (s, 3H), 1.57-4.77 (m, 4H).

### Example 15: N-(2-((4-(2-(4-chloro-2-fluorophenyl)-2-methylbenzo[d][1,3]dioxol-4-yl)piperidin-1-yl)methyl)-1-((S)-oxetan-2-ylmethyl)-1H-benzo[d]imidazol-5-yl)acetamide (Compound 115a)

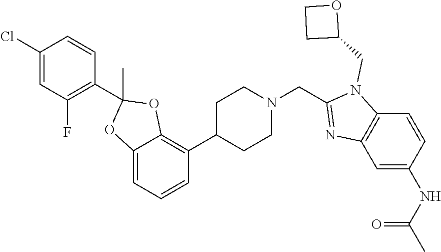 [SMILES: CC(=O)Nc1ccc2c(c1)nc(CN1CCC(c3cccc4c3OC(C)(c3ccc(Cl)cc3F)O4)CC1)n2C[C@@H]1CCO1]

N-(2-((4-(2-(4-Chloro-2-fluorophenyl)-2-methylbenzo[d][1,3]dioxol-4-yl)piperidin-1-yl)methyl)-1-((S)-oxetan-2-ylmethyl)-1H-benzo[d]imidazol-5-yl)acetamide Compound 115a is synthesized following the route in Compound 114a, using 4-(2-(4-chloro-2-fluorophenyl)-2-methylbenzo[d][1,3]dioxol-4-yl)piperidine in step E. MS Calcd.: 604.2; MS Found: 605.2 [M+H]⁺.

¹HNMR (400 MHz, DMSO-d₆) δ 9.87 (s, 1H), 7.89 (d, J=1.6 Hz, 1H), 7.53-7.59 (m, 2H), 7.51 (d, J=8.8 Hz, 1H), 7.30-7.35 (m, 2H), 6.78-6.79 (m, 2H), 6.72-6.76 (m, 1H), 5.06-5.13 (m, 1H), 4.66 (dd, J=15.2, 7.2 Hz, 1H), 4.50-4.60 (m, 1H), 4.42-4.49 (m, 1H), 4.35-4.40 (m, 1H), 3.87 (d, J=13.2 Hz, 1H), 3.72 (d, J=13.2 Hz, 1H), 2.99 (d, J=10.4 Hz, 1H), 2.86 (d, J=11.2 Hz, 1H), 2.60-2.72 (m, 2H), 2.39-2.46 (m, 1H), 2.11-2.25 (m, 2H), 2.03 (d, J=8.4 Hz, 6H), 1.67-1.81 (m, 4H).

### Example 17: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}oxetane-2-carboxamide (Compound 129a)

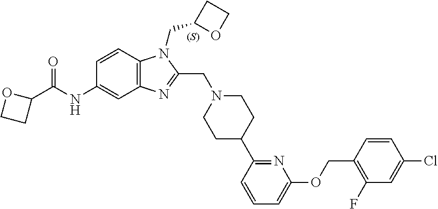

The synthesis of Compound 129a is similar to that of Compound 102a except oxetane-2-carboxylic acid is used instead of propionic acid. Compound 129a (3.08 mg, yield: 5%) is finally obtained as a white solid.

MS Calcd.: 619.2; MS Found: 620.1 [M+H]⁺.

¹H NMR (400 MHz, DMSO-d₆): 9.83 (s, 1H), 7.95-8.05 (m, 1H), 7.63 (t, J=7.2 Hz, 1H), 7.49-7.61 (m, 3H), 7.46 (dd, J=10.0, 2.0 Hz, 1H), 7.30 (dd, J=8.4 Hz, 1.6 Hz, 1H), 6.87 (d, J=7.2 Hz, 1H), 6.67 (d, J=8.0 Hz, 1H), 5.38 (s, 2H), 5.08-5.12 (m, 2H), 4.62-4.72 (m, 3H), 4.53-4.60 (m, 1H), 4.40-4.50 (m, 1H), 4.35-4.40 (m, 1H), 3.88 (d, J=13.2 Hz, 1H), 3.74 (d, J=13.2 Hz, 1H), 2.92-3.02 (m, 2H), 2.86 (d, J=14.4 Hz, 1H), 2.57-2.71 (m, 3H), 2.39-2.49 (m, 1H), 2.12-2.25 (m, 2H), 1.66-1.82 (m, 4H).

### Example 18: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2-phenylacetamide (Compound 130a)

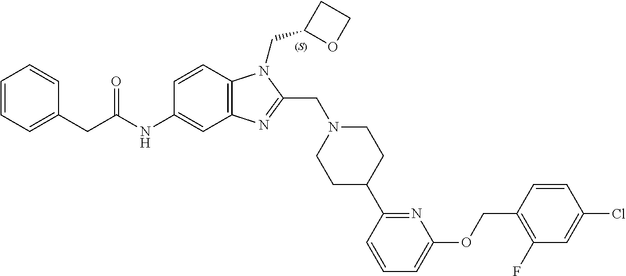

The synthesis of Compound 130a is similar to that of Compound 102a except 2-phenylacetic acid is used instead of propionic acid. Compound 130a (47.53 mg, yield: 78%) is finally obtained as a white solid.

MS Calcd.: 653.3; MS Found: 654.2 [M+H]⁺.

¹H NMR (400 MHz, DMSO-d₆): 10.11 (s, 1H), 7.91 (d, J=1.6 Hz, 1H), 7.62 (t, J=7.2 Hz, 1H), 7.51-7.60 (m, 2H), 7.46 (dd, J=10.0 Hz, 2.0 Hz, 1H), 7.22-7.37 (m, 7H), 6.87 (d, J=7.2 Hz, 1H), 6.67 (d, J=8.0 Hz, 1H), 5.37 (s, 2H), 5.06-5.12 (m, 1H), 4.62-4.72 (m, 1H), 4.53-4.57 (m, 1H), 4.43-4.49 (m, 1H), 4.34-4.39 (m, 1H), 3.87 (d, J=13.2 Hz, 1H), 3.73 (d, J=13.6 Hz, 1H), 3.64 (s, 2H), 2.97 (d, J=12.0 Hz, 1H), 2.84 (d, J=12.0 Hz, 1H), 2.56-2.71 (m, 2H), 2.35-2.48 (m, 1H), 2.11-2.23 (m, 2H), 1.60-1.82 (m, 4H).

### Example 19: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3,3,3-trifluoropropanamide (Compound 150a)

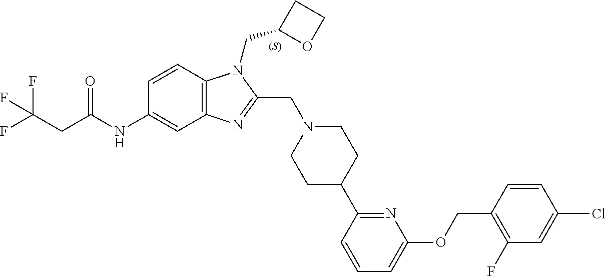

The synthesis of Compound 150a is similar to that of Compound 102a except 3,3,3-trifluoropropanoic acid is used instead of propionic acid. Compound 150a (20.1 mg, yield: 33.5%) is finally obtained as a white solid.

MS Calcd.: 645.2; MS Found: 646.1 [M+H]⁺.

¹H NMR (400 MHz, DMSO-d6) δ 10.25 (s, 1H), 7.88 (d, J=1.6 Hz, 1H), 7.63 (t, J=8.0 Hz, 1H), 7.54-7.58 (m, 2H), 7.46 (dd, J=10.0 Hz, 2.0 Hz, 1H), 7.25-7.33 (m, 2H), 6.87 (d, J=7.2 Hz, 1H), 6.67 (d, J=8.0 Hz, 1H), 5.38 (s, 2H), 5.07-5.13 (m, 1H), 4.66-4.72 (m, 1H), 4.55-4.61 (m, 1H), 4.42-4.50 (m, 1H), 4.35-4.41 (m, 1H), 3.87-3.91 (m, 1H), 3.73-3.76 (m, 1H), 3.50 (q, J=11.2 Hz, 2H), 2.97-3.02 (m, 1H), 2.84-2.87 (m, 1H), 2.57-2.72 (m, 2H), 2.38-2.47 (m, 1H), 2.13-2.28 (m, 2H), 1.67-1.83 (m, 4H).

### Example 20: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}cyclopentanecarboxamide (Compound 151a)

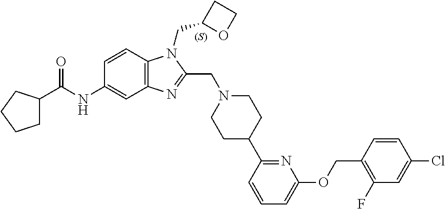

The synthesis of Compound 151a is similar to that of Compound 102a except cyclopentanecarboxylic acid is used instead of propionic acid. Compound 151a (22.0 mg, yield: 37%) is finally obtained as a white solid.

MS Calcd.: 631.3; MS Found: 632.2 [M+H]⁺.

¹H NMR (400 MHz, DMSO-d6) δ 9.79 (s, 1H), 7.91 (d, J=1.6 Hz, 1H), 7.63 (t, J=8.0 Hz, 1H), 7.56 (t, J=8.0 Hz, 1H), 7.50 (d, J=8.4 Hz, 1H), 7.46 (dd, J=10.0 Hz, 2.0 Hz, 1H), 7.36 (dd, J=8.4 Hz, 1.6 Hz, 1H), 7.29 (dd, J=8.4 Hz, 2.0 Hz, 1H), 6.87 (d, J=7.6 Hz, 1H), 6.67 (d, J=8.0 Hz, 1H), 5.37 (s, 2H), 5.06-5.12 (m, 1H), 4.64-4.71 (m, 1H), 4.53-4.60 (m, 1H), 4.42-4.49 (m, 1H), 4.34-4.39 (m, 1H), 3.86 (d, J=13.2 Hz, 1H), 3.73 (d, J=13.6 Hz, 1H), 2.94-2.99 (m, 1H), 2.82-2.88 (m, 1H), 2.72-2.79 (m, 1H), 2.57-2.69 (m, 2H), 2.39-2.46 (m, 1H), 2.12-2.24 (m, 2H), 1.66-1.88 (m, 10H), 1.53-1.59 (m, 2H).

### Example 21: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3,3-difluorocyclobutane-1-carboxamide (Compound 152a)

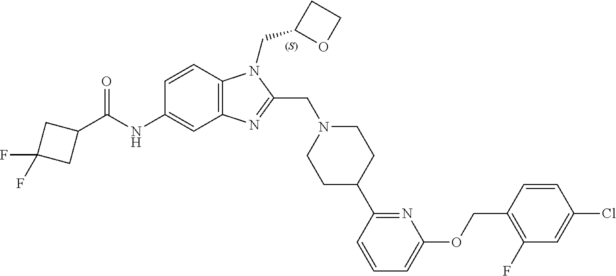

The synthesis of Compound 152a is similar to that of Compound 102a except 3,3-difluorocyclobutane-1-carboxylic acid is used instead of propionic acid. Compound 152a (23.34 mg, yield: 37%) is finally obtained as a white solid.

MS Calcd.: 653.2; MS Found: 653.9 [M+H]⁺.

¹H NMR (400 MHz, DMSO-d6) δ 10.04 (s, 1H), 7.91 (d, J=1.6 Hz, 1H), 7.63 (t, J=7.6 Hz, 1H), 7.53-7.58 (m, 2H), 7.46 (dd, J=10.0 Hz, 2.0 Hz, 1H), 7.35 (dd, J=8.8 Hz, 2.0 Hz, 1H), 7.29 (dd, J=8.4 Hz, 2.0 Hz, 1H), 6.87 (d, J=7.2 Hz, 1H), 6.67 (d, J=8.0 Hz, 1H), 5.38 (s, 2H), 5.07-5.13 (m, 1H), 4.65-4.71 (m, 1H), 4.53-4.58 (m, 1H), 4.42-4.49 (m, 1H), 4.35-4.40 (m, 1H), 3.88 (d, J=13.6 Hz, 1H), 3.73 (d, J=13.2 Hz, 1H), 3.06-3.13 (m, 1H), 2.94-2.99 (m, 1H), 2.76-2.86 (m, 5H), 2.56-2.70 (m, 2H), 2.38-2.44 (m, 1H), 2.12-2.24 (m, 2H), 1.63-1.82 (m, 4H).

### Example 22: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}oxane-4-carboxamide (Compound 153a)

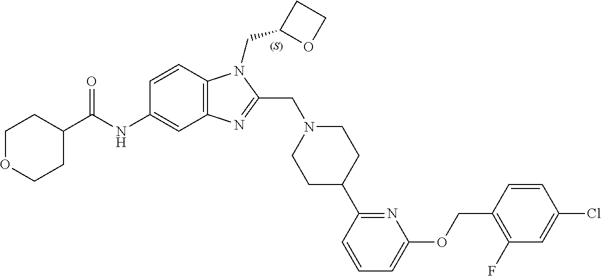

The synthesis of Compound 153a is similar to that of Compound 102a except tetrahydro-2H-pyran-4-carboxylic acid is used instead of propionic acid. Compound 153a (7.05 mg, yield: 11.8%) is finally obtained as a white solid.

MS Calcd.: 647.3; MS Found: 648.2 [M+H]⁺.

¹H NMR (400 MHz, DMSO-d6) δ 9.81 (s, 1H), 7.91 (d, J=1.6 Hz, 1H), 7.63 (t, J=7.6 Hz, 1H), 7.56 (t, J=8.0 Hz, 1H), 7.51 (d, J=8.8 Hz, 1H), 7.46 (dd, J=10.0 Hz, 2.0 Hz, 1H), 7.36 (dd, J=8.8 Hz, 2.0 Hz, 1H), 7.29 (dd, J=8.0 Hz, 2.0 Hz, 1H), 6.87 (d, J=7.6 Hz, 1H), 6.67 (d, J=8.0 Hz, 1H), 5.37 (s, 2H), 5.06-5.12 (m, 1H), 4.64-4.71 (m, 1H), 4.53-4.58 (m, 1H), 4.41-4.49 (m, 1H), 4.32-4.39 (m, 1H), 3.82-3.93 (m, 3H), 3.73 (d, J=13.6 Hz, 1H), 3.34-3.39 (m, 2H), 2.93-2.98 (m, 1H), 2.83-2.89 (m, 1H), 2.55-2.71 (m, 3H), 2.37-2.46 (m, 1H), 2.12-2.24 (m, 2H), 1.60-1.82 (m, 8H).

### Example 23: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}oxane-3-carboxamide (Compound 154a)

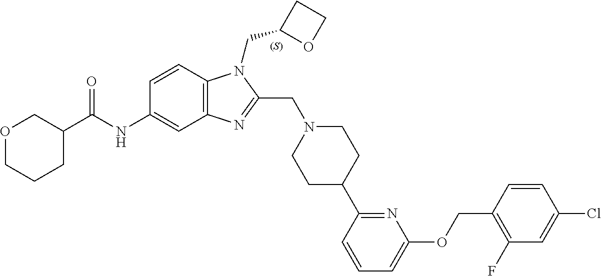

The synthesis of Compound 154a is similar to that of Compound 102a except tetrahydro-2H-pyran-3-carboxylic acid is used instead of propionic acid. Compound 154a (7.1 mg, yield: 4.9%) is finally obtained as a white solid.

MS Calcd.: 647.3; MS Found: 648.2 [M+H]⁺.

¹H NMR (400 MHz, METHANOL-D4) δ 7.92 (d, J=2.0 Hz, 1H), 7.58-7.54 (m, 2H), 7.49 (t, J=8.0 Hz, 1H), 7.40 (dd, J₁=2.0H, J₂=8.8 Hz, 1H), 7.22-7.16 (m, 2H), 6.81 (d, J=7.2 Hz, 1H), 6.62 (d, J=8.0 Hz, 1H), 5.41 (s, 2H), 5.28-5.22 (m, 1H), 4.81-4.75 (m, 1H), 4.68-4.59 (m, 2H), 4.47-4.42 (m, 1H), 4.07-4.03 (m, 1H), 3.97-3.82 (m, 3H), 3.58 (t, J=10.4 Hz, 1H), 3.49-3.42 (m, 1H), 3.06-3.01 (m, 1H), 2.94-2.89 (m, 1H), 2.80-2.61 (m, 3H), 2.54-2.49 (m, 1H), 2.33-2.20 (m, 2H), 2.08-2.02 (m, 1H), 1.92-1.80 (m, 5H), 1.74-1.67 (m, 2H).

¹⁹F NMR (400 MHz, METHANOL-D4) δ −117.67.

### Example 24: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}oxane-3-carboxamide (Compound 154b+Compound 154c

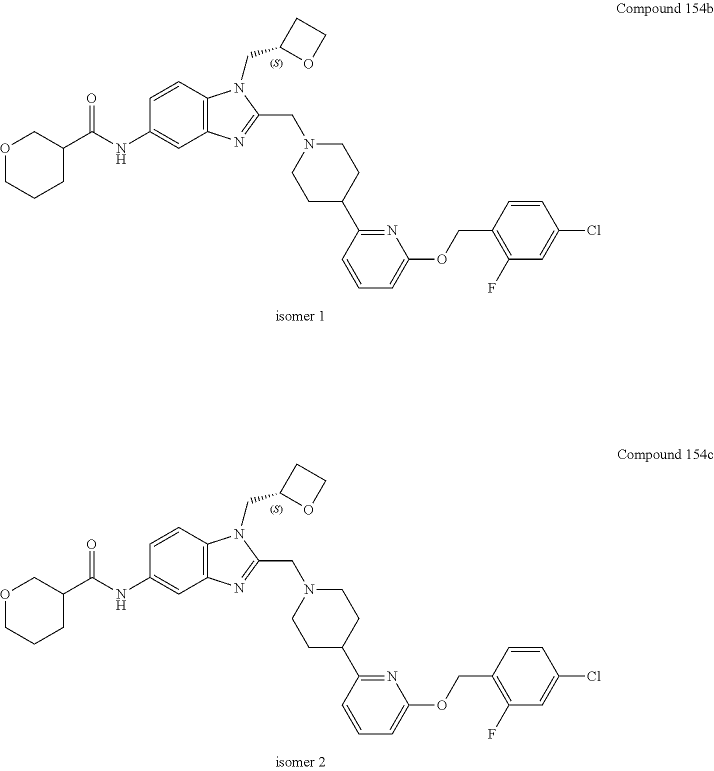

Chiral separation of Compound 154a (180 mg) using SFC (CO2/MeOH/DEA 60/40/0.04 2.8 ml/min, OD, 5 um, 4.6*250 (Daicel), 12 min) gave compound Compound 154b (isomer 1, 52.2 mg, yield: 10%) and Compound 154c (isomer 2, 46.2 mg, yield: 9%)

Compound 154c:

MS Calcd.: 647.3; MS Found: 648.3 [M+H]⁺.

¹H NMR (400 MHz, METHANOL-D4) δ 8.41 (s, 1H), 7.97 (d, J=1.6 Hz, 1H), 7.60-7.54 (m, 2H), 7.49 (t, J=8.0 Hz, 1H), 7.41 (dd, J=2.0 Hz, J=8.8 Hz, 1H), 7.23-7.17 (m, 2H), 6.84 (d, J=7.2 Hz, 1H), 6.65 (d, J=8.4 Hz, 1H), 5.41 (s, 2H), 5.27-5.21 (m, 1H), 4.81-4.75 (m, 1H), 4.67-4.60 (m, 2H), 4.47-4.41 (m, 1H), 4.11-3.98 (m, 3H), 3.92-3.89 (m, 1H), 3.58 (t, J=10.4 Hz, 1H), 3.49-3.42 (m, 1H), 3.18-3.10 (m, 1H), 3.09-3.06 (m, 1H), 2.82-2.64 (m, 3H), 2.56-2.39 (m, 3H), 2.09-2.03 (m, 1H), 1.94-1.82 (m, 5H), 1.74-1.66 (m, 2H).

¹⁹F NMR (400 MHz, METHANOL-D4) δ −117.66.

Compound 154b:

MS Calcd.: 647.3; MS Found: 648.3 [M+H]⁺.

¹H NMR (400 MHz, METHANOL-D4) δ 7.96 (d, J=2.0 Hz, 1H), 7.60-7.54 (m, 2H), 7.49 (t, J=8.0 Hz, 1H), 7.41 (dd, J=2.0 Hz, J=8.8 Hz, 1H), 7.23-7.17 (m, 2H), 6.83 (d, J=7.2 Hz, 1H), 6.65 (d, J=8.4 Hz, 1H), 5.41 (s, 2H), 5.27-5.22 (m, 1H), 4.82-4.75 (m, 1H), 4.67-4.59 (m, 2H), 4.49-4.42 (m, 1H), 4.06-4.02 (m, 2H), 3.96-3.89 (m, 2H), 3.58 (t, J=10.8 Hz, 1H), 3.49-3.42 (m, 1H), 3.13-3.10 (m, 1H), 3.03-2.98 (m, 1H), 2.82-2.64 (m, 3H), 2.56-2.31 (m, 3H), 2.05-2.03 (m, 1H), 1.92-1.82 (m, 5H), 1.74-1.68 (m, 2H).

¹⁹F NMR (400 MHz, METHANOL-D4) δ −117.67.

### Example 25: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}oxane-2-carboxamide (Compound 155a)

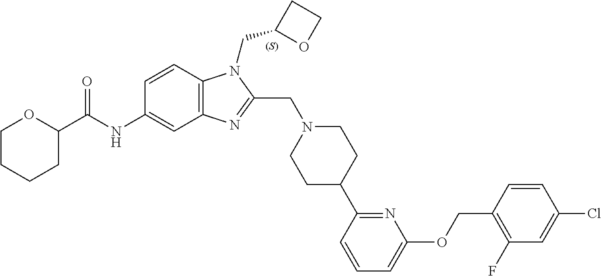

The synthesis of Compound 155a is similar to that of Compound 102a except tetrahydro-2H-pyran-3-carboxylic acid is used instead of propionic acid. Compound 155a (5.2 mg, yield: 4.2%) is finally obtained as a white solid.

MS Calcd.: 647.3; MS Found: 648.2 [M+H]⁺.

¹H NMR (400 MHz, METHANOL-D4) δ 7.97 (d, J=2.0 Hz, 1H), 7.59-7.55 (m, 2H), 7.49 (t, J=8.0 Hz, 1H), 7.40 (dd, J₁=1.6H, J₂=8.8 Hz, 1H), 7.22-7.17 (m, 2H), 6.81 (d, J=6.8 Hz, 1H), 6.62 (d, J=8.4 Hz, 1H), 5.41 (s, 2H), 5.28-5.22 (m, 1H), 4.82-4.78 (m, 1H), 4.69-4.60 (m, 2H), 4.47-4.42 (m, 1H), 4.17-4.14 (m, 1H), 3.96 (t, J=2.8 Hz, 1H), 3.94-3.83 (m, 2H), 3.67-3.53 (m, 1H), 3.05-3.01 (m, 1H), 2.94-2.90 (m, 1H), 2.82-2.74 (m, 1H), 2.67-2.60 (m, 1H), 2.58-2.582 (m, 1H), 2.33-2.20 (m, 2H), 2.11-2.06 (m, 1H), 1.98-1.92 (m, 1H), 1.90-1.80 (m, 4H), 1.69-1.52 (m, 4H).

¹⁹F NMR (400 MHz, METHANOL-D4) δ −117.67.

### Example 26: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-1,1-dioxo-1λ⁶-thiane-4-carboxamide (Compound 156a)

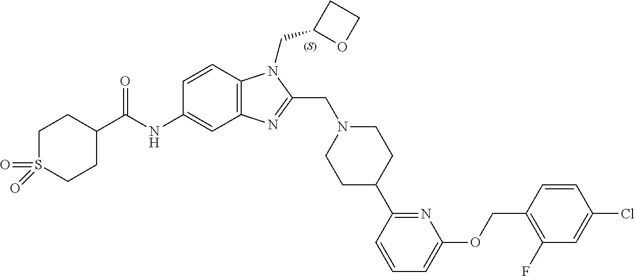

The synthesis of Compound 156a is similar to that of Compound 102a except tetrahydro-2H-thiopyran-4-carboxylic acid 1,1-dioxide is used instead of propionic acid. Compound 156a (2.7 mg, yield: 4.2%) is finally obtained as a white solid.

MS Calcd.: 695.2; MS Found: 696.2 [M+H]⁺.

¹H NMR (400 MHz, METHANOL-D4) 67.91 (d, J=2.0 Hz, 1H), 7.59-7.55 (m, 2H), 7.49 (t, J=8.0 Hz, 1H), 7.40 (dd, J₁=2.0H, J₂=8.8 Hz, 1H), 7.22-7.16 (m, 2H), 6.81 (d, J=7.2 Hz, 1H), 6.62 (d, J=8.4 Hz, 1H), 5.41 (s, 2H), 5.26-5.20 (m, 1H), 4.80-4.78 (m, 1H), 4.68-4.59 (m, 2H), 4.47-4.40 (m, 1H), 3.96 (d, J=14.0 Hz, 1H), 3.84 (d, J=13.6 Hz, 1H), 3.22-3.13 (m, 4H), 3.05-3.02 (m, 1H), 2.93-2.91 (m, 1H), 2.80-2.61 (m, 3H), 2.56-2.49 (m, 1H), 2.38-2.23 (m, 6H), 1.90-1.79 (m, 4H).

¹⁹F NMR (400 MHz, METHANOL-D4) δ −117.670

### Example 27: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}benzamide (Compound 166a)

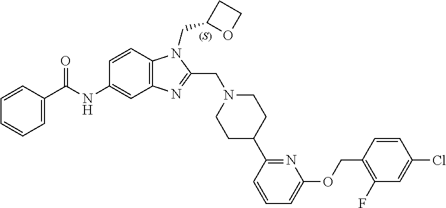

The synthesis of Compound 166a is similar to that of Compound 102a except benzoic acid is used instead of propionic acid. Compound 166a (19.69 mg, yield: 33%) is finally obtained as a white solid.

MS Calcd.: 639.2; MS Found: 640.1 [M+H]⁺.

¹H NMR (400 MHz, DMSO-d₆): 10.21 (s, 1H), 8.05 (s, 1H), 7.96-8.00 (m, 2H), 7.50-7.65 (m, 7H), 7.46 (dd, J=10.0 Hz, 2.0 Hz, 1H), 7.30 (dd, J=8.0 Hz, 1.6 Hz, 1H), 6.88 (d, J=7.2 Hz, 1H), 6.68 (d, J=8.4 Hz, 1H), 5.38 (s, 2H), 5.09-5.15 (m, 1H), 4.68-4.74 (m, 1H), 4.57-4.61 (m, 1H), 4.43-4.51 (m, 1H), 4.36-4.42 (m, 1H), 3.90 (d, J=13.6 Hz, 1H), 3.76 (d, J=13.6 Hz, 1H), 2.98-3.01 (m, 1H), 2.86-2.90 (m, 1H), 2.58-2.73 (m, 2H), 2.40-2.47 (m, 1H), 2.12-2.25 (m, 2H), 1.64-1.83 (m, 4H).

### Example 28: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2,2,2-trifluoroacetamide (Compound 167a)

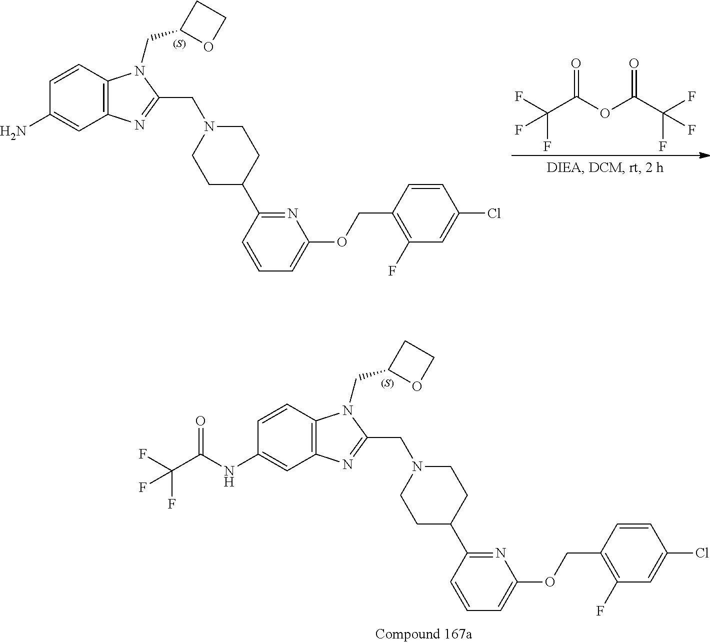

To a solution of (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (50 mg, 0.093 mmol) in DCM (2 mL) w as added DIEA (12 mg, 0.093 mmol) and 2,2,2-trifluoroacetic anhydride (39 mg, 0.187 mmol) at 0° C. The resulting mixture was stirred at room temperature for 2 hours. The reaction mixture was quenched with H₂O and extracted with DCM. The organic layers were combined and concentrated under vacuum. The residue was purified by Prep-HPLC to give Compound 167a (18.66 mg, yield: 31.6%) as a white solid.

MS Calcd.: 631.2; MS Found: 632.0 [M+H]⁺.

¹H NMR (400 MHz, DMSO-d₆): 11.21 (s, 1H), 7.91 (d, J=2.0 Hz, 1H), 7.54-7.66 (m, 3H), 7.44-7.48 (m, 2H), 7.29 (dd, J=8.0 Hz, 1.6 Hz, 1H), 6.87 (d, J=7.2 Hz, 1H), 6.67 (d, J=8.4 Hz, 1H), 5.38 (s, 2H), 5.08-5.14 (m, 1H), 4.69-4.75 (m, 1H), 4.57-4.62 (m, 1H), 4.44-4.50 (m, 1H), 4.36-4.41 (m, 1H), 3.90 (d, J=13.6 Hz, 1H), 3.76 (d, J=13.6 Hz, 1H), 2.95-3.02 (m, 1H), 2.84-2.87 (m, 1H), 2.55-2.71 (m, 2H), 2.39-2.47 (m, 1H), 2.13-2.28 (m, 2H), 1.62-1.82 (m, 4H).

### Example 29: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}pyridine-3-carboxamide (Compound 168a)

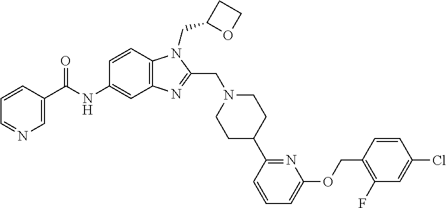 [SMILES: O=C(Nc1ccc2c(c1)nc(CN1CCC(c3cccc(OCc4ccc(Cl)cc4F)n3)CC1)n2C[C@@H]1CCO1)c1cccnc1]

The synthesis of Compound 168a is similar to that of Compound 102a except nicotinic acid is used instead of propionic acid. Compound 168a (2.1 mg, yield: 3.5%) is finally obtained as a white solid.

MS Calcd.: 640.2; MS Found: 641.2 [M+H]⁺.

¹H NMR (400 MHz, MeOH-d₄) δ 9.11 (d, J=1.6 Hz, 1H), 8.72 (d, J=5.2 Hz, 1H), 8.40-8.36 (m, 1H), 8.07 (d, J=1.6 Hz, 1H), 7.65-7.55 (m, 4H), 7.49 (d, J=8.0 Hz, 1H), 7.22-7.17 (m, 2H), 6.82 (d, J=7.2 Hz, 1H), 6.62 (d, J=8.4 Hz, 1H), 5.42 (s, 2H), 5.30-5.25 (m, 1H), 4.81-4.64 (m, 3H), 4.49-4.44 (m, 1H), 3.98 (d, J=13.6 Hz, 1H), 3.96 (d, J=13.6 Hz, 1H), 3.09-3.04 (m, 1H), 2.98-2.90 (m, 1H), 2.82-2.76 (m, 1H), 2.63-2.60 (m, 1H), 2.60-2.51 (m, 1H), 2.35-2.22 (m, 2H), 1.87-1.82 (m, 4H).

¹⁹F NMR (400 MHz, MeOH-d₄)) δ −117.659

### Example 30: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3-cyanobenzamide (Compound 169a)

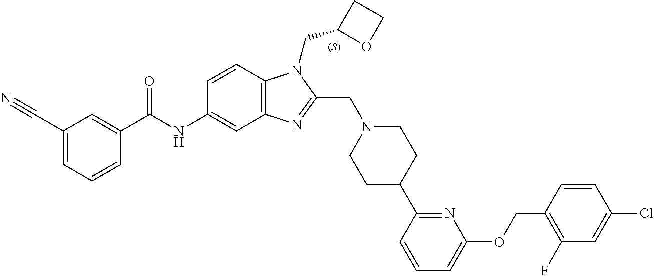

The synthesis of Compound 169a is similar to that of Compound 102a except 3-cyanobenzoic acid is used instead of propionic acid. Compound 169a (14.74 mg, yield: 24%) is finally obtained as a white solid.

MS Calcd.: 664.2; MS Found: 665.5 [M+H]⁺.

¹H NMR (400 MHz, DMSO-d₆): 10.40 (s, 1H), 8.42-8.43 (m, 1H), 8.28 (d, J=8.0 Hz, 1H), 8.03-8.08 (m, 2H), 7.76 (t, J=8.0 Hz, 1H), 7.53-7.65 (m, 4H), 7.46 (dd, J=10.0 Hz, 2.0 Hz, 1H), 7.30 (dd, J=8.4 Hz, 2.0 Hz, 1H), 6.88 (d, J=7.2 Hz, 1H), 6.68 (d, J=8.0 Hz, 1H), 5.38 (s, 2H), 5.09-5.15 (m, 1H), 4.69-4.74 (m, 1H), 4.57-4.62 (m, 1H), 4.45-4.51 (m, 1H), 4.37-4.42 (m, 1H), 3.91 (d, J=13.2 Hz, 1H), 3.76 (d, J=13.6 Hz, 1H), 2.95-3.02 (m, 1H), 2.84-2.91 (m, 1H), 2.55-2.74 (m, 2H), 2.40-2.47 (m, 1H), 2.12-2.26 (m, 2H), 1.67-1.83 (m, 4H).

### Example 31: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}pyridine-2-carboxamide (Compound 170a)

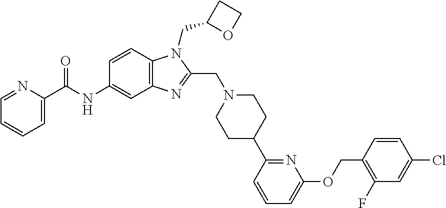 [SMILES: O=C(Nc1ccc2c(c1)nc(CN1CCC(c3cccc(OCc4ccc(Cl)cc4F)n3)CC1)n2C[C@@H]1CCO1)c1ccccn1]

The synthesis of Compound 170a is similar to that of Compound 102a except picolinic acid is used instead of propionic acid. Compound 170a (13.1 mg, yield: 15.2%) is finally obtained as a white solid.

MS Calcd.: 640.2; MS Found: 641.7 [M+H]⁺.

¹H NMR (400 MHz, DMSO-d₆): δ10.62 (s, 1H), 8.76 (d, J=4.4 Hz, 1H), 8.20 (d, J=9.2 Hz, 2H), 8.10-8.06 (m, 1H), 7.72-7.55 (m, 5H), 7.47 (d, J=10.0 Hz, 1H), 7.31 (d, J=7.6 Hz, 1H), 6.89 (d, J=7.6 Hz, 1H), 6.69 (d, J=8.0 Hz, 1H), 5.38 (s, 2H), 5.13-5.10 (m, 1H), 4.74-4.67 (m, 1H), 4.62-4.55 (m, 1H), 4.51-4.45 (m, 1H), 4.42-4.37 (m, 1H), 3.92 (d, J=13.2 Hz, 1H), 3.78 (d, J=13.6 Hz, 1H), 3.01 (d, J=10.8 Hz, 1H), 2.89 (d, J=10.8 Hz, 1H), 2.74-2.65 (m, 1H), 2.65-2.55 (m, 1H), 2.47-2.40 (m, 1H), 2.25-2.12 (m, 2H), 1.86-1.65 (m, 4H).

### Example 32: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}pyridine-4-carboxamide (Compound 171a)

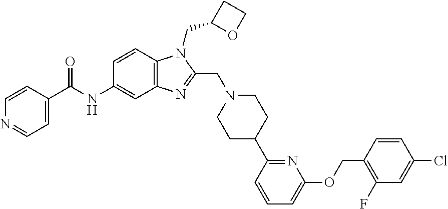 [SMILES: O=C(Nc1ccc2c(c1)nc(CN1CCC(c3cccc(OCc4ccc(Cl)cc4F)n3)CC1)n2C[C@@H]1CCO1)c1ccncc1]

The synthesis of Compound 171a is similar to that of Compound 102a except isonicotinic acid is used instead of propionic acid. Compound 171a (9.81 mg, yield: 16%) is finally obtained as a white solid.

MS Calcd.: 640.2; MS Found: 641.5 [M+H]⁺.

¹H NMR (400 MHz, DMSO-d6) δ 10.47 (s, 1H), 8.78-8.81 (m, 2H), 8.05 (d, J=1.6 Hz, 1H), 7.88-7.92 (m, 2H), 7.52-7.65 (m, 4H), 7.46 (dd, J=10.0 Hz, 2.0 Hz, 1H), 7.30 (dd, J=8.4 Hz, 2.0 Hz, 1H), 6.88 (d, J=7.2 Hz, 1H), 6.68 (d, J=8.0 Hz, 1H), 5.38 (m, 2H), 5.09-5.15 (m, 1H), 4.68-4.74 (m, 1H), 4.57-4.61 (m, 1H), 4.45-4.50 (m, 1H), 4.36-4.42 (m, 1H), 3.90 (d, J=13.6 Hz, 1H), 3.76 (d, J=13.2 Hz, 1H), 2.98-3.01 (m, 1H), 2.86-2.90 (m, 1H), 2.58-2.73 (m, 2H), 2.40-2.48 (m, 1H), 2.12-2.26 (m, 2H), 1.67-1.83 (m, 4H).

### Example 33: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-1,4-dioxane-2-carboxamide (Compound 172a)

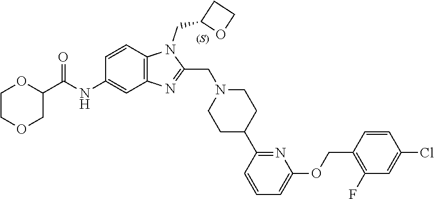

The synthesis of Compound 172a is similar to that of Compound 102a except 1,4-dioxane-2-carboxylic acid is used instead of propionic acid. Compound 172a (4.0 mg, yield: 7%) is finally obtained as a white solid.

MS Calcd.: 649.2; MS Found: 650.5[M+H]⁺.

¹H NMR (400 MHz, DMSO-d6) δ 9.67 (s, 1H), 7.92 (d, J=1.6 Hz, 1H), 7.63 (t, J=8.0 Hz, 1H), 7.52-7.58 (m, 2H), 7.44-7.48 (m, 2H), 7.29 (dd, J=8.4 Hz, 1.6 Hz, 1H), 6.87 (d, J=7.6 Hz, 1H), 6.67 (d, J=8.0 Hz, 1H), 5.37 (s, 2H), 5.07-5.12 (m, 1H), 4.65-4.71 (m, 1H), 4.54-4.58 (m, 1H), 4.43-4.49 (m, 1H), 4.36-4.40 (m, 1H), 4.22 (dd, J=9.6 Hz, 3.2 Hz, 1H), 3.96 (dd, J=11.2 Hz, 2.8 Hz, 1H), 3.86-3.92 (m, 2H), 3.68-3.76 (m, 3H), 3.53-3.60 (m, 2H), 2.96-2.99 (m, 1H), 2.84-2.88 (m, 1H), 2.56-2.70 (m, 2H), 2.41-2.46 (m, 1H), 2.11-2.24 (m, 2H), 1.66-1.81 (m, 4H).

### Example 34: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-6-methylpyridine-3-carboxamide (Compound 173a)

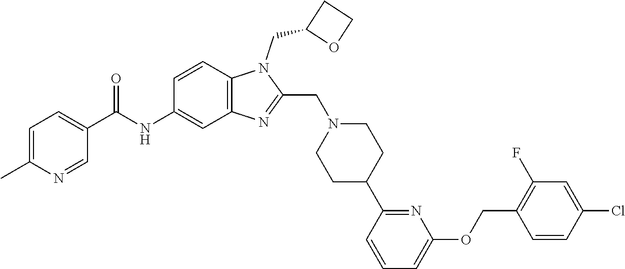 [SMILES: Cc1ccc(C(=O)Nc2ccc3c(c2)nc(CN2CCC(c4cccc(OCc5ccc(Cl)cc5F)n4)CC2)n3C[C@@H]2CCO2)cn1]

The synthesis of Compound 173a is similar to that of Compound 102a except 6-methylnicotinic acid is used instead of propionic acid. Compound 173a (23.05 mg, yield: 37%) is finally obtained as a white solid.

MS Calcd.: 654.2; MS Found: 655.4 [M+H]⁺.

¹H NMR (400 MHz, DMSO-d₆): 10.31 (s, 1H), 9.02 (d, J=2.0 Hz, 1H), 8.22 (dd, J=8.0 Hz, 2.4 Hz, 1H), 8.04 (d, J=1.6 Hz, 1H), 7.53-7.65 (m, 4H), 7.46 (dd, J=10.0 Hz, 2.0 Hz, 1H), 7.42 (d, J=8.0 Hz, 1H), 7.30 (dd, J=8.0 Hz, 1.6 Hz, 1H), 6.88 (d, J=7.2 Hz, 1H), 6.68 (d, J=8.4 Hz, 1H), 5.38 (s, 2H), 5.09-5.15 (m, 1H), 4.68-4.74 (m, 1H), 4.56-4.61 (m, 1H), 4.45-4.51 (m, 1H), 4.37-4.42 (m, 1H), 3.90 (d, J=13.6 Hz, 1H), 3.75 (d, J=13.6 Hz, 1H), 2.98-3.01 (m, 1H), 2.86-2.89 (m, 1H), 2.67-2.73 (m, 1H), 2.54-2.62 (m, 4H), 2.40-2.49 (m, 1H), 2.14-2.25 (m, 2H), 1.64-1.82 (m, 4H).

### Example 35: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-6-ethylpyridine-3-carboxamide (Compound 174a)

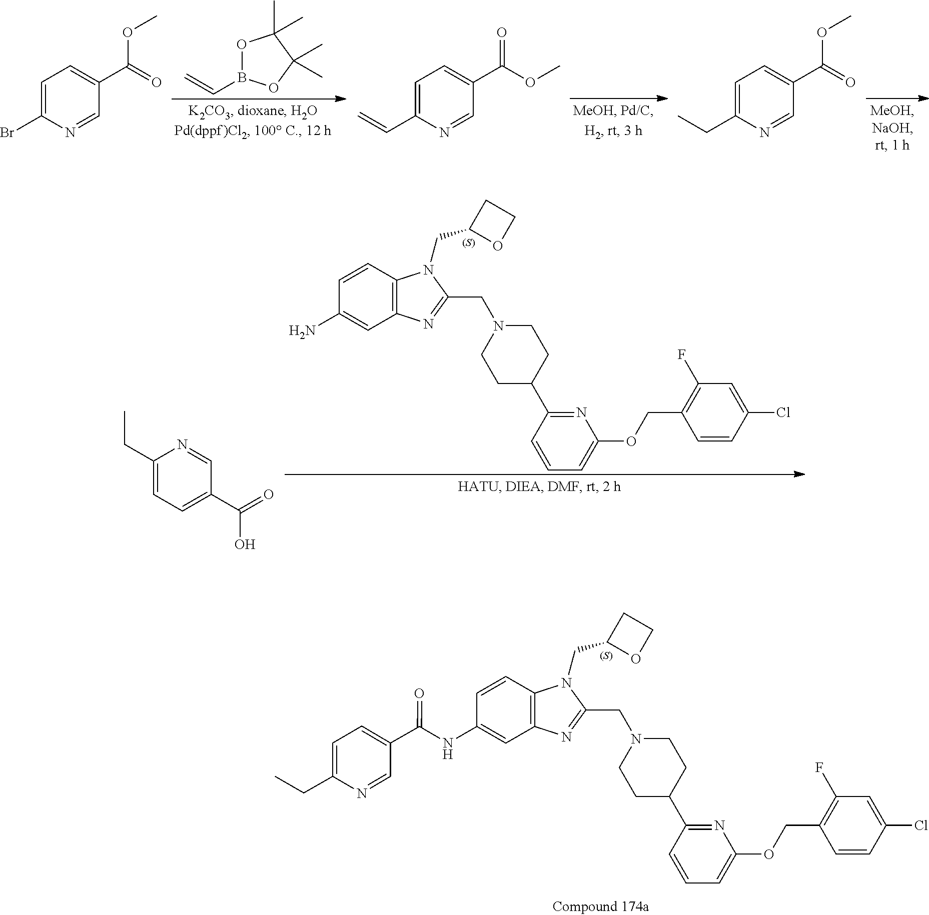

### Step A: methyl 6-vinylnicotinate

To a mixture of methyl 6-bromonicotinate (500 mg, 2.31 mmol), 4,4,5,5-tetramethyl-2-vinyl-1,3,2-dioxaborolane (712 mg, 4.62 mmol), K₂CO₃ (956 mg, 6.93 mmol) in dioxane/H₂O (10 mL/1 mL) was added Pd(dppf)Cl₂ (169 mg, 0.231 mmol) at room temperature. The mixture was heated at 100° C. for 12 h under N₂ atmosphere. The reaction was quenched by water, extracted with EA. The organic layers were combined, dried over Na₂SO₄, filtered and concentrated under vacuum. The residue was purified by silica gel column and eluted with PE:EtOAc=10:1 to give methyl 6-vinylnicotinate (300 mg, 86% yield) as colorless oil.

MS Calcd.: 163.1; MS Found: 164.1 [M+H]⁺.

### Step B: methyl 6-ethylnicotinate

To a solution of methyl 6-vinylnicotinate (300 mg, 1.84 mmol) in MeOH (10 mL) was added Pd/C (10% w/w, 30 mg) at room temperature. The mixture was stirred at room temperature under hydrogen atmosphere for 3 h. The reaction was filtered and the filtrate was concentrated under vacuum to give methyl 6-ethylnicotinate (260 mg, 87% yield) as colorless oil.

MS Calcd.: 165.1; MS Found: 166.3 [M+H]⁺.

### Step C: 6-ethylnicotinic acid

A mixture of methyl 6-ethylnicotinate (150 mg, 0.71 mmol), NaOH (85 mg, 2.13 mmol) in MeOH (2 mL) and H₂O (0.5 mL) was stirred at room temperature for 1 hour. The reaction mixture was adjusted to pH=6 with con. HCl, extracted with EA. The organic layers were combined and concentrated under vacuum to give 6-ethylnicotinic acid (120 mg, yield: 51%) as a white solid.

MS Calcd.: 151.1; MS Found: 152.2 [M+H]⁺.

### Step D: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-6-ethylpyridine-3-carboxamide (Compound 174a)

The synthesis of Compound 174a is similar to that of Compound 102a except 6-ethylnicotinic acid is used instead of propionic acid. Compound 174a (22 mg, yield: 36%) is finally obtained as a white solid.

MS Calcd.: 668.3; MS Found: 669.4[M+H]⁺.

¹H NMR (400 MHz, DMSO-d₆): δ 10.32 (s, 1H), 9.04 (d, J=2.0 Hz, 1H), 8.23 (dd, J=8.4 Hz, 2.4 Hz, 1H), 8.04 (d, J=1.2 Hz, 1H), 7.53-7.65 (m, 4H), 7.42-7.48 (m, 2H), 7.30 (dd, J=8.4 Hz, 2.0 Hz, 1H), 6.87 (d, J=7.2 Hz, 1H), 6.67 (d, J=8.0 Hz, 1H), 5.38 (s, 2H), 5.08-5.14 (m, 1H), 4.68-4.74 (m, 1H), 4.57-4.61 (m, 1H), 4.48-4.52 (m, 1H), 4.36-4.42 (m, 1H), 3.90 (d, J=13.6 Hz, 1H), 3.75 (d, J=13.6 Hz, 1H), 2.98-3.01 (m, 1H), 2.81-2.89 (m, 3H), 2.58-2.73 (m, 2H), 2.40-2.48 (m, 1H), 2.12-2.25 (m, 2H), 1.65-1.83 (m, 4H), 1.27 (t, J=7.6 Hz, 3H).

### Example 36: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-6-methoxypyridine-3-carboxamide (Compound 175a)

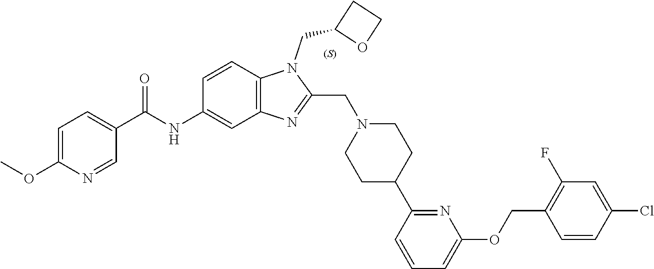

The synthesis of Compound 175a is similar to that of Compound 102a except 6-methoxynicotinic acid is used instead of propionic acid. Compound 175a (28.50 mg, yield: 45%) is finally obtained as a white solid.

MS Calcd.: 670.2; MS Found: 671.4 [M+H]⁺.

¹H NMR (400 MHz, DMSO-d₆): 10.21 (s, 1H), 8.81 (d, J=2.0 Hz, 1H), 8.26 (dd, J=8.8 Hz, 1.6 Hz, 1H), 8.02 (d, J=1.6 Hz, 1H), 7.51-7.65 (m, 4H), 7.46 (dd, J=10.0 Hz, 2.0 Hz, 1H), 7.30 (dd, J=8.0 Hz, 1.6 Hz, 1H), 6.96 (d, J=8.8 Hz, 1H), 6.88 (d, J=7.6 Hz, 1H), 6.68 (d, J=8.0 Hz, 1H), 5.38 (s, 2H), 5.09-5.15 (m, 1H), 4.68-4.74 (m, 1H), 4.56-4.61 (m, 1H), 4.45-4.50 (m, 1H), 4.36-4.42 (m, 1H), 3.94 (s, 3H), 3.90 (d, J=13.6 Hz, 1H), 3.76 (d, J=13.2 Hz, 1H), 2.98-3.01 (m, 1H), 2.84-2.88 (m, 1H), 2.57-2.73 (m, 2H), 2.40-2.46 (m, 1H), 2.13-2.26 (m, 2H), 1.67-1.83 (m, 4H).

### Example 37: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-6-cyclopropylpyridine-3-carboxamide (Compound 176a)

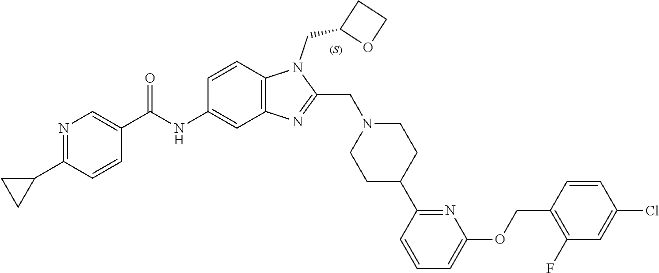

The synthesis of Compound 176a is similar to that of Compound 102a except 6-cyclopropylnicotinic acid is used instead of propionic acid. Compound 176a (25.72 mg, yield: 40%) is finally obtained as a white solid.

MS Calcd.: 680.3; MS Found: 681.4 [M+H]⁺.

¹H NMR (400 MHz, DMSO-d₆) δ 10.26 (s, 1H), 8.96 (d, J=2.0 Hz, 1H), 8.17 (dd, J=8.4 Hz, 2.4 Hz, 1H), 8.03 (d, J=1.2 Hz, 1H), 7.52-7.65 (m, 4H), 7.42-7.48 (m, 2H), 7.30 (dd, J=8.0 Hz, 1.6 Hz, 1H), 6.88 (d, J=7.2 Hz, 1H), 6.67 (d, J=8.0 Hz, 1H), 5.38 (s, 2H), 5.09-5.15 (m, 1H), 4.68-4.74 (m, 1H), 4.56-4.61 (m, 1H), 4.45-4.50 (m, 1H), 4.36-4.41 (m, 1H), 3.90 (d, J=13.2 Hz, 1H), 3.75 (d, J=13.6 Hz, 1H), 2.96-3.01 (m, 1H), 2.86-2.90 (m, 1H), 2.57-2.73 (m, 2H), 2.40-2.48 (m, 1H), 2.13-2.25 (m, 3H), 1.64-1.83 (m, 4H), 0.97-1.05 (m, 4H).

### Example 38: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-6-(2-methoxyethoxy)pyridine-3-carboxamide (Compound 177a)

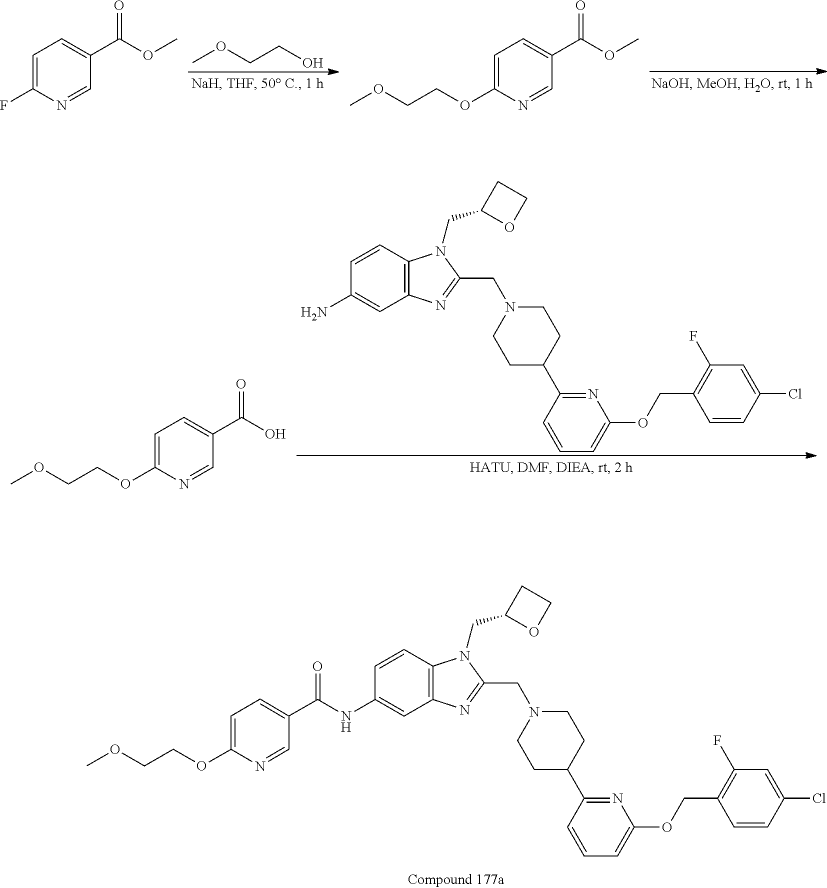

### Step A: methyl 6-(2-methoxyethoxy)nicotinate

To a solution of 2-methoxyethan-1-ol (200 mg, 2.6 mmol) in THF (3 mL) was added NaH (150 mg, 3.75 mmol) at room temperature. The mixture was heated to 50° C. for 0.5 h, then added with methyl 6-fluoronicotinate (367 mg, 2.3 mmol). The mixture was stirred at 50° C. for 0.5 hours. The reaction was quenched by water, extracted with EA. The organic layers were combined, dried over Na₂SO₄, filtered and concentrated under vacuum. The residue was purified by gel column and eluted with PE:EtOAc=3:1 to give methyl 6-(2-methoxyethoxy)nicotinate (150 mg, 28% yield) as colorless oil.

MS Calcd.: 211.1; MS Found: 212.2 [M+H]⁺.

### Step B: 6-(2-methoxyethoxy)nicotinic acid

A mixture of methyl 6-(2-methoxyethoxy)nicotinate (150 mg, 0.71 mmol), NaOH (85 mg, 2.13 mmol) in MeOH (2 mL) and H₂O (0.5 mL) was stirred at room temperature for 1 hours. The reaction mixture was adjusted to pH=6 with con. HCl, extracted with EA. The organic layers were combined and concentrated under vacuum to give 6-(2-methoxyethoxy)nicotinic acid (120 mg, yield: 85.7%) as a white solid.

MS Calcd.: 197.1; MS Found: 198.1 [M+H]⁺.

### Step C: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-6-(2-methoxyethoxy)pyridine-3-carboxamide (Compound 177a)

The synthesis of Compound 177a is similar to that of Compound 102a except 6-(2-methoxyethoxy)nicotinic acid is used instead of propionic acid. Compound 177a (12 mg, yield: 15%) is finally obtained as a white solid.

MS Calcd.: 714.3; MS Found: 715.4[M+H]⁺.

¹H NMR (400 MHz, DMSO-d₆): δ10.21 (s, 1H), 8.78 (d, J=2.4 Hz, 1H), 8.26 (dd, J=8.8 Hz, 2.4 Hz, 1H), 8.02 (d, J=1.6 Hz, 1H), 7.51-7.65 (m, 4H), 7.46 (dd, J=10.0 Hz, 2.4 Hz, 1H), 7.30 (dd, J=8.0 Hz, 1.6 Hz, 1H), 6.96 (d, J=8.8 Hz, 1H), 6.87 (d, J=7.6 Hz, 1H), 6.67 (d, J=8.0 Hz, 1H), 5.38 (s, 2H), 5.09-5.15 (m, 1H), 4.68-4.74 (m, 1H), 4.56-4.61 (m, 1H), 4.45-4.50 (m, 3H), 4.36-4.41 (m, 1H), 3.89 (d, J=13.6 Hz, 1H), 3.75 (d, J=13.6 Hz, 1H), 3.68-3.70 (m, 2H), 3.31 (s, 3H), 2.98-3.02 (m, 1H), 2.86-2.90 (m, 1H), 2.57-2.73 (m, 2H), 2.40-2.46 (m, 1H), 2.14-2.25 (m, 2H), 1.65-1.83 (m, 4H)

### Example 39: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3-methylpyridine-4-carboxamide (Compound 178a)

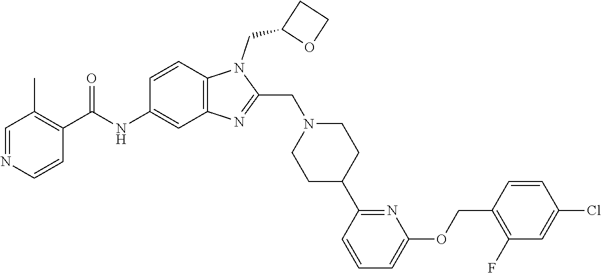 [SMILES: Cc1cnccc1C(=O)Nc1ccc2c(c1)nc(CN1CCC(c3cccc(OCc4ccc(Cl)cc4F)n3)CC1)n2C[C@@H]1CCO1]

The synthesis of Compound 178a is similar to that of Compound 102a except 3-methylpyridine-4-carboxylic acid is used instead of propionic acid. Compound 178a (13.22 mg, yield: 26%) is finally obtained as a white solid.

MS Calcd.: 654.2; MS Found: 655.4 [M+H]⁺.

¹H NMR (400 MHz, DMSO-d₆) δ 10.43 (s, 1H), 8.57 (s, 1H), 8.54 (d, J=4.8 Hz, 1H), 8.03 (d, J=1.2 Hz, 1H), 7.52-7.65 (m, 3H), 7.44-7.52 (m, 3H), 7.30 (dd, J=8.4 Hz, 1.6 Hz, 1H), 6.88 (d, J=7.2 Hz, 1H), 6.68 (d, J=8.4 Hz, 1H), 5.38 (s, 2H), 5.09-5.14 (m, 1H), 4.68-4.73 (m, 1H), 4.57-4.62 (m, 1H), 4.43-4.50 (m, 1H), 4.35-4.41 (m, 1H), 3.90 (d, J=13.2 Hz, 1H), 3.77 (d, J=13.2 Hz, 1H), 2.95-3.02 (m, 1H), 2.84-2.90 (m, 1H), 2.58-2.73 (m, 2H), 2.38-2.47 (m, 1H), 2.38 (s, 3H), 2.12-2.28 (m, 2H), 1.65-1.83 (m, 4H).

### Example 40: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}imidazo[1,5-a]pyridine-7-carboxamide (Compound 179a)

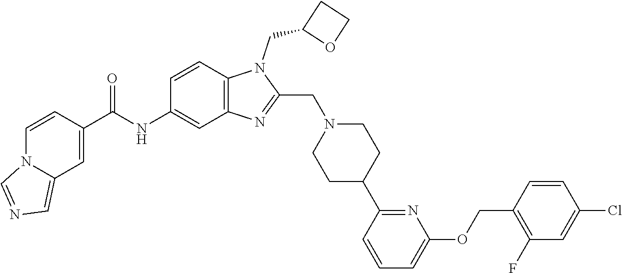 [SMILES: O=C(Nc1ccc2c(c1)nc(CN1CCC(c3cccc(OCc4ccc(Cl)cc4F)n3)CC1)n2C[C@@H]1CCO1)c1ccn2cncc2c1]

The synthesis of Compound 179a is similar to that of Compound 102a except imidazo[1,5-a]pyridine-7-carboxylic acid is used instead of propionic acid Compound 179a (3.58 mg, yield: 7%) is finally obtained as a white solid.

MS Calcd.: 679.2; MS Found: 680.4 [M+H]⁺.

¹H NMR (400 MHz, DMSO-d₆) δ 10.25 (s, 1H), 8.52 (s, 1H), 8.42 (d, J=7.2 Hz, 1H), 8.33 (s, 1H), 8.04 (s, 1H), 7.52-7.67 (m, 5H), 7.46 (dd, J=7.2, 1.6 Hz, 1H), 7.30 (dd, J=6.4, 1.6 Hz, 1H), 7.18 (dd, J=7.2 Hz, 1.6 Hz, 1H), 6.88 (d, J=7.2 Hz, 1H), 6.68 (d, J=8.4 Hz, 1H), 5.38 (s, 2H), 5.08-5.14 (m, 1H), 4.68-4.74 (m, 1H), 4.55-4.61 (m, 1H), 4.45-4.51 (m, 1H), 4.37-4.42 (m, 1H), 3.81-3.90 (m, 1H), 3.70-3.72 (m, 1H), 2.96-3.05 (m, 1H), 2.84-2.92 (m, 1H), 2.56-2.74 (m, 2H), 2.40-2.47 (m, 1H), 2.08-2.28 (m, 2H), 1.65-1.83 (m, 4H).

### Example 41: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-5H,6H,7H-cyclopenta[c]pyridine-6-carboxamide (Compound 180a)

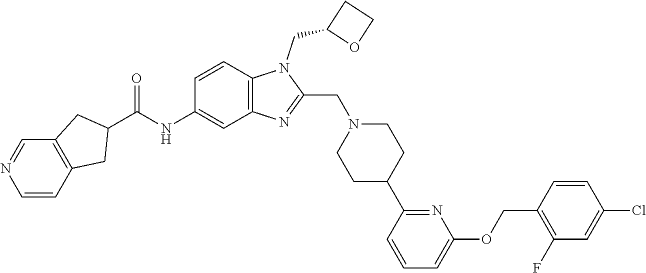 [SMILES: O=C(Nc1ccc2c(c1)nc(CN1CCC(c3cccc(OCc4ccc(Cl)cc4F)n3)CC1)n2C[C@@H]1CCO1)C1Cc2ccncc2C1]

The synthesis of is similar to that of (3.88 (d, J=13.6 Hz, 1H), 3.73 (d, J=13.2 Hz, 1H), 3.41-3.47 (m, 1H), 3.15-3.27 (m, 4H), 2.96-3.01 (m, 1H), 2.84-2.87 (m, 1H), 2.55-2.71 (m, 2H), 2.33-2.45 (m, 1H), 2.12-2.24 (m, 2H), 1.66-1.82 (m, 4H).

### Example 42: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}imidazo[1,2-a]pyridine-3-carboxamide (Compound 181a)

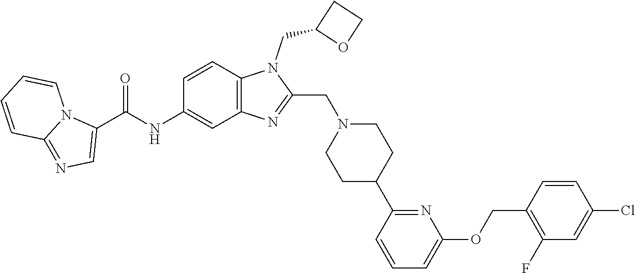 [SMILES: O=C(Nc1ccc2c(c1)nc(CN1CCC(c3cccc(OCc4ccc(Cl)cc4F)n3)CC1)n2C[C@@H]1CCO1)c1cnc2ccccn12]

The synthesis of Compound 181a is similar to that of Compound 102a except imidazo[1,2-a]pyridine-3-carboxylic acid is used instead of propionic acid. Compound 181a (24.24 mg, yield: 38%) is finally obtained as a white solid.

MS Calcd.: 679.2; MS Found: 680.1 [M+H]⁺.

¹H NMR (400 MHz, DMSO-d₆) δ 10.19 (s, 1H), 9.51 (d, J=6.8 Hz, 1H), 8.60 (s, 1H), 8.02 (d, J=1.6 Hz, 1H), 7.76 (d, J=9.2 Hz, 1H), 7.49-7.65 (m, 5H), 7.47 (dd, J=10.0 Hz, 2.0 Hz, 1H), 7.30 (dd, J=8.0 Hz, 1.6 Hz, 1H), 7.16-7.19 (m, 1H), 6.88 (d, J=7.2 Hz, 1H), 6.68 (d, J=8.4 Hz, 1H), 5.38 (s, 2H), 5.09-5.16 (m, 1H), 4.69-4.75 (m, 1H), 4.57-4.62 (m, 1H), 4.46-4.51 (m, 1H), 4.38-4.43 (m, 1H), 3.91 (d, J=13.6 Hz, 1H), 3.76 (d, J=13.2 Hz, 1H), 2.93-3.04 (m, 1H), 2.85-2.91 (m, 1H), 2.58-2.72 (m, 2H), 2.40-2.50 (m, 1H), 2.14-2.26 (m, 2H), 1.68-1.84 (m, 4H).

### Example 43: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2-(1-methyl-1H-pyrazol-4-yl)propanamide (Compound 182a)

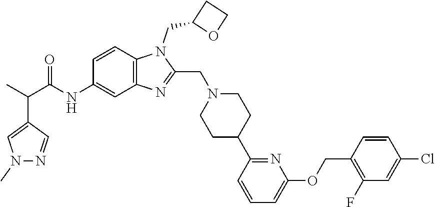 [SMILES: CC(C(=O)Nc1ccc2c(c1)nc(CN1CCC(c3cccc(OCc4ccc(Cl)cc4F)n3)CC1)n2C[C@@H]1CCO1)c1cnn(C)c1]

The synthesis of Compound 182a is similar to that of Compound 102a except 2-(1-methyl-1H-pyrazol-4-yl)propanoic acid is used instead of propionic acid. Compound 182a (13.57 mg, yield: 21.6%) is finally obtained as a white solid.

MS Calcd.: 671.3; MS Found: 672.4 [M+H]⁺.

¹H NMR (400 MHz, DMSO-d₆) δ 9.91 (s, 1H), 7.91 (s, 1H), 7.61-7.64 (m, 1H), 7.50-7.58 (m, 3H), 7.45 (dd, J=10.0 Hz, 2.0 Hz, 1H), 7.32-7.36 (m, 2H), 7.29 (dd, J=8.0 Hz, 1.6 Hz, 1H), 6.87 (d, J=7.2 Hz, 1H), 6.67 (d, J=8.0 Hz, 1H), 5.37 (s, 2H), 5.06-5.12 (m, 1H), 4.62-4.71 (m, 1H), 4.52-4.57 (m, 1H), 4.43-4.49 (m, 1H), 4.33-4.39 (m, 1H), 3.87 (d, J=13.6 Hz, 1H), 3.78 (s, 3H), 3.68-3.75 (m, 2H), 2.95-2.98 (m, 1H), 2.81-2.87 (m, 1H), 2.53-2.71 (m, 2H), 2.39-2.44 (m, 1H), 2.12-2.23 (m, 2H), 1.61-1.81 (m, 4H), 1.37 (d, J=7.2 Hz, 3H).

### Example 44: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2-methyloxane-2-carboxamide (Compound 183a)

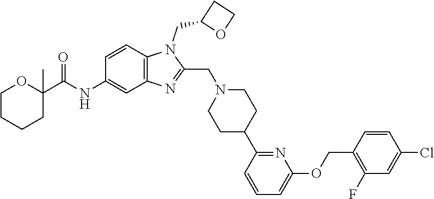 [SMILES: CC1(C(=O)Nc2ccc3c(c2)nc(CN2CCC(c4cccc(OCc5ccc(Cl)cc5F)n4)CC2)n3C[C@@H]2CCO2)CCCCO1]

The synthesis of Compound 183a is similar to that of Compound 102a except 2-methyltetrahydro-2H-pyran-2-carboxylic acid is used instead of propionic acid. Compound 183a (6.7 mg, yield: 10.8%) is finally obtained as a white solid.

MS Calcd.: 661.3; MS Found: 662.3 [M+H]⁺.

¹H NMR (400 MHz, DMSO-d₆) δ 9.59 (s, 1H), 7.97 (s, 1H), 7.60-7.65 (m, 1H), 7.56 (t, J=8.4 Hz, 1H), 7.50-7.53 (m, 2H), 7.46 (dd, J=10.0 Hz, 2.0 Hz, 1H), 7.29 (dd, J=8.4 Hz, 2.0 Hz, 1H), 6.87 (d, J=7.2 Hz, 1H), 6.67 (d, J=8.0 Hz, 1H), 5.38 (s, 2H), 5.05-5.13 (m, 1H), 4.63-4.71 (m, 1H), 4.52-4.61 (m, 1H), 4.42-4.49 (m, 1H), 4.33-4.39 (m, 1H), 3.73-3.89 (m, 3H), 3.52-3.58 (m, 1H), 2.93-2.98 (m, 1H), 2.82-2.87 (m, 1H), 2.53-2.71 (m, 2H), 2.37-2.46 (m, 1H), 2.11-2.23 (m, 3H), 1.60-1.82 (m, 5H), 1.42-1.51 (m, 3H), 1.32-1.41 (m, 4H).

### Example 45: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3,5-dimethylpyridine-2-carboxamide (Compound 184a)

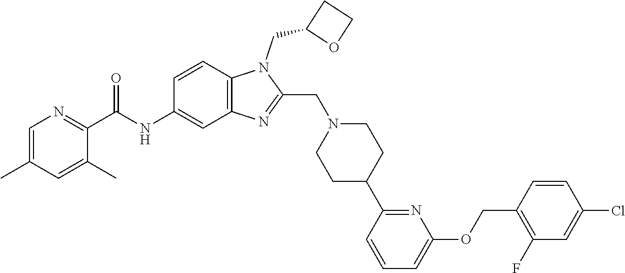 [SMILES: Cc1cnc(C(=O)Nc2ccc3c(c2)nc(CN2CCC(c4cccc(OCc5ccc(Cl)cc5F)n4)CC2)n3C[C@@H]2CCO2)c(C)c1]

The synthesis of Compound 184a is similar to that of Compound 102a except 3,5-dimethylpicolinic acid is used instead of propionic acid. Compound 184a (17.96 mg, yield: 28%) is finally obtained as a white solid.

MS Calcd.: 668.3; MS Found: 669.4 [M+H]⁺.

¹H NMR (400 MHz, DMSO-d₆) δ 10.45 (s, 1H), 8.38 (s, 1H), 8.12 (s, 1H), 7.55-7.67 (m, 5H), 7.46 (d, J=9.6 Hz, 1H), 7.30 (d, J=8.0 Hz, 1H), 6.88 (d, J=7.2 Hz, 1H), 6.68 (d, J=8.0 Hz, 1H), 5.39 (s, 2H), 5.08-5.14 (m, 1H), 4.68-4.74 (m, 1H), 4.57-4.63 (m, 1H), 4.44-4.52 (m, 1H), 4.35-4.42 (m, 1H), 3.90 (d, J=13.2 Hz, 1H), 3.76 (d, J=13.6 Hz, 1H), 2.97-3.02 (m, 1H), 2.82-2.92 (m, 1H), 2.55-2.73 (m, 5H), 2.41-4.49 (m, 1H), 2.37 (s, 3H), 2.12-2.28 (m, 2H), 1.62-1.83 (m, 4H).

### Example 46: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2-(5-methyl-1H-pyrazol-1-yl)propanamide (Compound 185a)

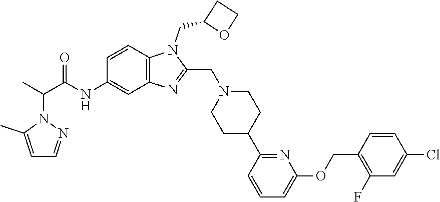 [SMILES: Cc1ccnn1C(C)C(=O)Nc1ccc2c(c1)nc(CN1CCC(c3cccc(OCc4ccc(Cl)cc4F)n3)CC1)n2C[C@@H]1CCO1]

The synthesis of Compound 185a is similar to that of Compound 102a except 2-(5-methyl-1H-pyrazol-1-yl)propanoic acid is used instead of propionic acid.

Compound 185a (10.06 mg, yield: 16%) is finally obtained as a white solid.

MS Calcd.: 671.3; MS Found: 672.8 [M+H]⁺.

¹H NMR (400 MHz, DMSO-d₆) δ 9.93 (s, 1H), 7.87 (d, J=2.0 Hz, 1H), 7.60-7.64 (m, 1H), 7.53-7.58 (m, 2H), 7.46 (dd, J=10.0 Hz, 2.0 Hz, 1H), 7.28-7.35 (m, 3H), 6.87 (d, J=7.2 Hz, 1H), 6.67 (d, J=8.4 Hz, 1H), 6.08 (d, J=0.8 Hz, 1H), 5.37 (s, 2H), 5.06-5.14 (m, 2H), 4.65-4.71 (m, 1H), 4.52-4.59 (m, 1H), 4.42-4.49 (m, 1H), 4.32-4.39 (m, 1H), 3.87 (d, J=13.6 Hz, 1H), 3.73 (d, J=13.2 Hz, 1H), 2.94-3.01 (m, 1H), 2.80-2.88 (m, 1H), 2.56-2.71 (m, 2H), 2.39-2.48 (m, 1H), 2.28 (s, 3H), 2.10-2.23 (m, 2H), 1.62-1.81 (m, 7H).

### Example 47: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3,3-difluorocyclopentane-1-carboxamide (Compound 186a)

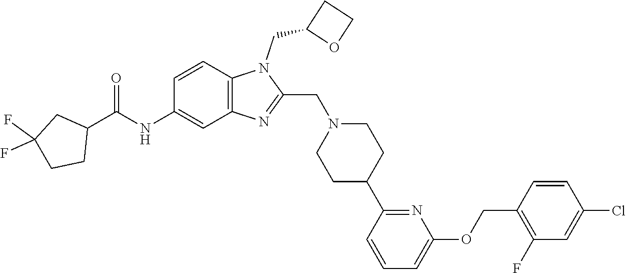 [SMILES: O=C(Nc1ccc2c(c1)nc(CN1CCC(c3cccc(OCc4ccc(Cl)cc4F)n3)CC1)n2C[C@@H]1CCO1)C1CCC(F)(F)C1]

The synthesis of Compound 186a is similar to that of Compound 102a except 3,3-difluorocyclopentane-1-carboxylic acid is used instead of propionic acid. Compound 186a (14.49 mg, yield: 23%) is finally obtained as a white solid.

MS Calcd.: 667.2; MS Found: 668.5 [M+H]⁺.

¹H NMR (400 MHz, DMSO-d₆) δ 9.98 (s, 1H), 7.93 (s, 1H), 7.50-7.64 (m, 3H), 7.45 (d, J=10.0 Hz, 1H), 7.37 (d, J=8.4 Hz, 1H), 7.29 (d, J=8.0 Hz, 1H), 6.87 (d, J=7.2 Hz, 1H), 6.67 (d, J=8.0 Hz, 1H), 5.38 (s, 2H), 5.05-5.12 (m, 1H), 4.62-4.71 (m, 1H), 4.52-4.58 (m, 1H), 4.42-4.49 (m, 1H), 4.32-4.41 (m, 1H), 3.88 (d, J=13.2 Hz, 1H), 3.74 (d, J=13.2 Hz, 1H), 3.05-3.15 (m, 1H), 2.96-3.01 (m, 1H), 2.80-2.89 (m, 1H), 2.55-2.74 (m, 2H), 2.29-2.49 (m, 3H), 2.05-2.23 (m, 5H), 1.89-2.01 (m, 1H), 1.64-1.81 (m, 4H).

### Example 48: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-1,5-dimethyl-1H-pyrazole-3-carboxamide (Compound 187a)

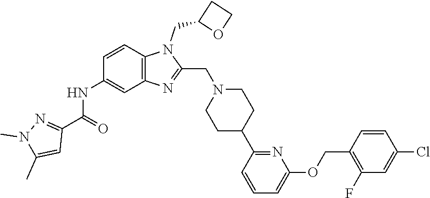 [SMILES: Cc1cc(C(=O)Nc2ccc3c(c2)nc(CN2CCC(c4cccc(OCc5ccc(Cl)cc5F)n4)CC2)n3C[C@@H]2CCO2)nn1C]

The synthesis of Compound 187a is similar to that of Compound 102a except 1,5-dimethyl-1H-pyrazole-3-carboxylic acid is used instead of propionic acid. Compound 187a (10.24 mg, yield: 16.8%) is finally obtained as a white solid.

MS Calcd.: 657.3; MS Found: 658.3 [M+H]⁺.

¹H NMR (400 MHz, DMSO-d₆) δ 9.85 (s, 1H), 8.07 (s, 1H), 7.50-7.65 (m, 4H), 7.46 (d, J=9.6 Hz, 1H), 7.30 (d, J=6.8 Hz, 1H), 6.87 (d, J=6.4 Hz, 1H), 6.67 (d, J=7.6 Hz, 1H), 6.56 (s, 1H), 5.38 (s, 2H), 5.06-5.15 (m, 1H), 4.53-4.72 (m, 2H), 4.39-4.51 (m, 2H), 3.73-3.90 (m, 5H), 2.95-3.01 (m, 1H), 2.82-2.91 (m, 1H), 2.58-2.71 (m, 2H), 2.38-2.47 (m, 1H), 2.32 (s, 3H), 2.13-2.26 (m, 2H), 1.62-1.82 (m, 4H).

### Example 49: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2-methylpyridine-3-carboxamide (Compound 188a)

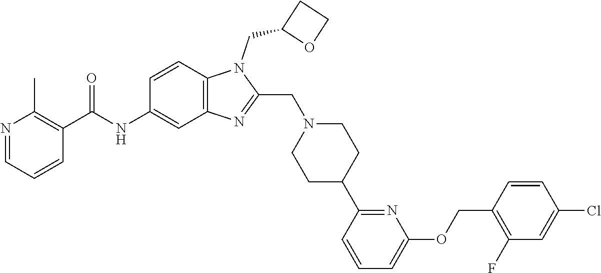 [SMILES: Cc1ncccc1C(=O)Nc1ccc2c(c1)nc(CN1CCC(c3cccc(OCc4ccc(Cl)cc4F)n3)CC1)n2C[C@@H]1CCO1]

The synthesis of Compound 188a is similar to that of Compound 102a except 2-methylnicotinic acid is used instead of propionic acid. Compound 188a (40 mg, yield: 65%) is finally obtained as a white solid.

MS Calcd.: 654.2; MS Found: 655.4 [M+H]⁺.

¹H NMR (400 MHz, DMSO-d₆) δ 10.38 (s, 1H), 8.56 (d, J=4.4 Hz, 1H), 8.04 (s, 1H), 7.88 (d, J=7.2 Hz, 1H), 7.53-7.65 (m, 3H), 7.51 (d, J=8.8 Hz, 1H), 7.46 (d, J=9.6 Hz, 1H), 7.34-7.39 (m, 1H), 7.30 (d, J=8.0 Hz, 1H), 6.88 (d, J=7.2 Hz, 1H), 6.68 (d, J=8.0 Hz, 1H), 5.39 (s, 2H), 5.09-5.13 (m, 1H), 4.68-4.74 (m, 1H), 4.57-4.64 (m, 1H), 4.45-4.51 (m, 1H), 4.35-4.41 (m, 1H), 3.90 (d, J=13.6 Hz, 1H), 3.77 (d, J=13.2 Hz, 1H), 2.90-3.05 (m, 1H), 2.84-2.91 (m, 1H), 2.59-2.73 (m, 5H), 2.40-2.48 (m, 1H), 2.11-2.26 (m, 2H), 1.60-1.83 (m, 4H).

### Example 50: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-4,4-difluorooxane-3-carboxamide (Compound 189a)

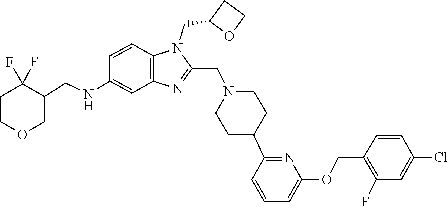 [SMILES: Fc1cc(Cl)ccc1COc1cccc(C2CCN(Cc3nc4cc(NCC5COCCC5(F)F)ccc4n3C[C@@H]3CCO3)CC2)n1]

The synthesis of Compound 189a is similar to that of Compound 102a except 4,4-difluorotetrahydro-2H-pyran-3-carboxylic acid is used instead of propionic acid. Compound 189a (21.68 mg, yield: 34%) is finally obtained as a white solid.

MS Calcd.: 683.2; MS Found: 684.4 [M+H]⁺.

¹H NMR (400 MHz, DMSO-d₆) δ 10.07 (s, 1H), 7.89-7.90 (d, J=1.2 Hz, 1H), 7.63 (t, J=7.6 Hz, 1H), 7.52-7.58 (m, 2H), 7.46 (dd, J=12.0 Hz, 2.0 Hz, 1H), 7.34 (dd, J=8.8 Hz, 2.0 Hz, 1H), 7.29 (dd, J=8.4 Hz, 2.0 Hz, 1H), 6.87 (d, J=7.2 Hz, 1H), 6.67 (d, J=8.0 Hz, 1H), 5.38 (s, 2H), 5.07-5.13 (m, 1H), 4.63-4.71 (m, 1H), 4.52-4.59 (m, 1H), 4.44-4.49 (m, 1H), 4.32-4.39 (m, 1H), 3.90-4.02 (m, 1H), 3.80-3.89 (m, 3H), 3.62-3.76 (m, 2H), 3.07-3.18 (m, 1H), 2.93-2.99 (m, 1H), 2.82-2.89 (m, 1H), 2.52-2.69 (m, 2H), 2.37-2.47 (m, 1H), 2.28-2.2.37 (m, 1H), 2.12-2.24 (m, 2H), 1.93-2.06 (m, 1H), 1.66-1.82 (m, 4H).

### Example 51: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-5-fluoropyridine-2-carboxamide (Compound 190a)

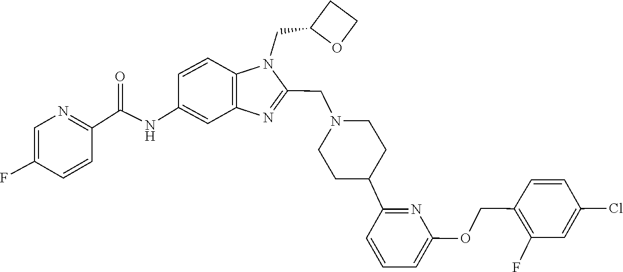 [SMILES: O=C(Nc1ccc2c(c1)nc(CN1CCC(c3cccc(OCc4ccc(Cl)cc4F)n3)CC1)n2C[C@@H]1CCO1)c1ccc(F)cn1]

The synthesis of Compound 190a is similar to that of Compound 102a except 5-fluoropicolinic acid is used instead of propionic acid. Compound 190a (11.25 mg, yield: 19.6%) is finally obtained as a white solid.

MS Calcd.: 658.2; MS Found: 659.3 [M+H]⁺.

¹H NMR (400 MHz, DMSO-d₆) δ 10.57 (s, 1H), 8.74 (d, J=2.8 Hz, 1H), 8.26 (dd, J=12.8 Hz, 4.4 Hz, 1H), 8.16 (d, J=2.0 Hz, 1H), 7.96-8.01 (m, 1H), 7.55-7.70 (m, 4H), 7.46 (dd, J=10.0, 2.0 Hz, 1H), 7.30 (dd, J=8.4 Hz, 2.0 Hz, 1H), 6.88 (d, J=7.2 Hz, 1H), 6.68 (d, J=8.4 Hz, 1H), 5.38 (s, 2H), 5.08-5.15 (m, 1H), 4.68-4.73 (m, 1H), 4.57-4.61 (m, 1H), 4.42-4.50 (m, 1H), 4.38-4.42 (m, 1H), 3.90 (d, J=13.2 Hz, 1H), 3.76 (d, J=13.2 Hz, 1H), 2.95-3.03 (m, 1H), 2.84-2.92 (m, 1H), 2.55-2.73 (m, 2H), 2.40-2.47 (m, 1H), 2.14-2.26 (m, 2H), 1.65-1.82 (m, 4H).

### Example 52: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3,5-dimethylpyridine-4-carboxamide (Compound 191a)

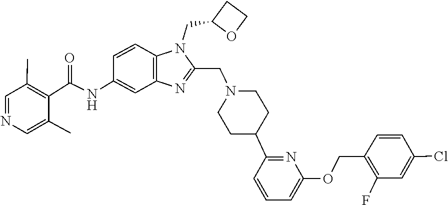 [SMILES: Cc1cncc(C)c1C(=O)Nc1ccc2c(c1)nc(CN1CCC(c3cccc(OCc4ccc(Cl)cc4F)n3)CC1)n2C[C@@H]1CCO1]

The synthesis of Compound 191a is similar to that of Compound 102a except 3,5-dimethylisonicotinic acid is used instead of propionic acid. Compound 191a (14.3 mg, yield: 23%) is finally obtained as a white solid.

MS Calcd.: 668.3; MS Found: 669.5 [M+H]⁺.

¹H NMR (400 MHz, DMSO-d₆) δ 10.45 (s, 1H), 8.37 (s, 2H), 8.00 (d, J=2.0 Hz, 1H), 7.52-7.65 (m, 3H), 7.44-7.49 (m, 2H), 7.30 (dd, J=8.4 Hz, 2.0 Hz, 1H), 6.87 (d, J=7.2 Hz, 1H), 6.67 (d, J=8.0 Hz, 1H), 5.38 (s, 2H), 5.08-5.14 (m, 1H), 4.67-4.73 (m, 1H), 4.55-4.61 (m, 1H), 4.44-4.50 (m, 1H), 4.34-4.40 (m, 1H), 3.89 (d, J=13.2 Hz, 1H), 3.77 (d, J=13.2 Hz, 1H), 2.96-3.01 (m, 1H), 2.85-2.90 (m, 1H), 2.57-2.72 (m, 3H), 2.29 (s, 6H), 2.13-2.24 (m, 2H), 1.67-1.82 (m, 4H).

### Example 53: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-6,6-dimethyloxane-3-carboxamide (Compound 192a)

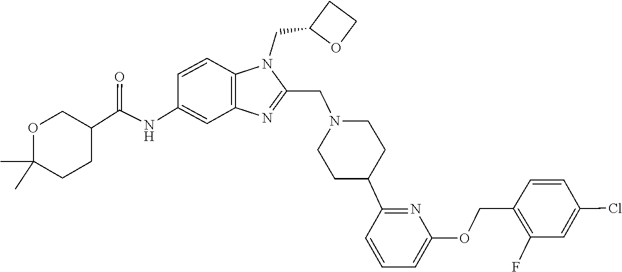 [SMILES: CC1(C)CCC(C(=O)Nc2ccc3c(c2)nc(CN2CCC(c4cccc(OCc5ccc(Cl)cc5F)n4)CC2)n3C[C@@H]2CCO2)CO1]

The synthesis of Compound 192a is similar to that of Compound 102a except 6,6-dimethyltetrahydro-2H-pyran-3-carboxylic acid is used instead of propionic acid. Compound 192a (6.08 mg, yield: 9.6%) is finally obtained as a white solid.

MS Calcd.: 675.3; MS Found: 676.4 [M+H]⁺.

¹H NMR (400 MHz, DMSO-d₆) δ 9.86 (s, 1H), 7.87-7.91 (m, 1H), 7.63 (t, J=7.6 Hz, 1H), 7.56 (t, J=8.0 Hz, 1H), 7.51 (d, J=8.8 Hz, 1H), 7.46 (dd, J=10.0 Hz, 2.0 Hz, 1H), 7.30-7.35 (m, 1H), 7.29 (dd, J=8.0 Hz, 1.6 Hz, 1H), 6.87 (d, J=7.2 Hz, 1H), 6.67 (d, J=8.0 Hz, 1H), 5.37 (s, 2H), 5.07-5.12 (m, 1H), 4.64-4.72 (m, 1H), 4.53-4.59 (m, 1H), 4.42-4.49 (m, 1H), 4.33-4.39 (m, 1H), 3.87 (d, J=13.2 Hz, 1H), 3.62-3.76 (m, 3H), 2.94-3.01 (m, 1H), 2.80-2.88 (m, 1H), 2.50-2.69 (m, 3H), 2.36-2.46 (m, 1H), 2.10-2.23 (m, 2H), 1.62-1.92 (m, 6H), 1.54-1.61 (m, 1H), 1.35-1.44 (m, 1H), 1.18 (s, 3H), 1.15 (s, 3H).

### Example 54: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}spiro[2.3]hexane-1-carboxamide (Compound 193a)

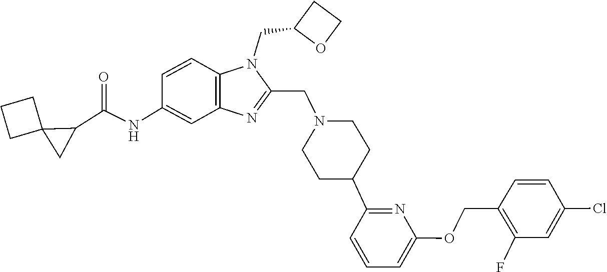 [SMILES: O=C(Nc1ccc2c(c1)nc(CN1CCC(c3cccc(OCc4ccc(Cl)cc4F)n3)CC1)n2C[C@@H]1CCO1)C1CC12CCC2]

The synthesis of Compound 193a is similar to that of Compound 102a except spiro[2.3]hexane-1-carboxylic acid is used instead of propionic acid. Compound 193a (15.74 mg, yield: 26%) is finally obtained as a white solid.

MS Calcd.: 643.3; MS Found: 644.5 [M+H]⁺.

¹H NMR (400 MHz, DMSO-d₆) δ 10.02 (s, 1H), 7.91 (s, 1H), 7.63 (t, J=8.0 Hz, 1H), 7.56 (t, J=8.4 Hz, 1H), 7.51 (d, J=8.8 Hz, 1H), 7.45 (d, J=10.0 Hz, 1H), 7.37 (d, J=8.8 Hz, 1H), 7.29 (d, J=8.0 Hz, 1H), 6.87 (d, J=7.2 Hz, 1H), 6.67 (d, J=8.0 Hz, 1H), 5.38 (s, 2H), 5.05-5.15 (m, 1H), 4.65-4.71 (m, 1H), 4.52-4.59 (m, 1H), 4.42-4.49 (m, 1H), 4.33-4.41 (m, 1H), 3.87 (d, J=13.2 Hz, 1H), 3.74 (d, J=13.2 Hz, 1H), 2.94-3.01 (m, 1H), 2.80-2.86 (m, 1H), 2.57-2.74 (m, 2H), 2.38-2.45 (m, 1H), 2.12-2.24 (m, 6H), 1.93-2.07 (m, 2H), 1.63-1.82 (m, 5H), 1.08 (t, J=4.4 Hz, 1H), 0.90-0.95 (m, 1H).

### Example 55: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2-fluoro-2-(1H-pyrazol-1-yl)acetamide (Compound 194a)

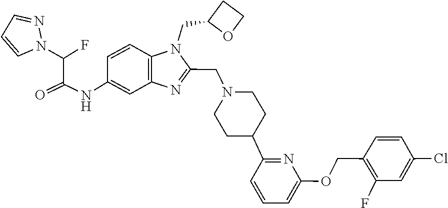 [SMILES: O=C(Nc1ccc2c(c1)nc(CN1CCC(c3cccc(OCc4ccc(Cl)cc4F)n3)CC1)n2C[C@@H]1CCO1)C(F)n1cccn1]

The synthesis of Compound 194a is similar to that of Compound 102a except 2-fluoro-2-(1H-pyrazol-1-yl)acetic acid is used instead of propionic acid. Compound 194a (16.13 mg, yield: 26%) is finally obtained as a white solid.

MS Calcd.: 661.2; MS Found: 662.4 [M+H]⁺.

¹H NMR (400 MHz, DMSO-d₆) δ 10.55 (s, 1H), 8.13 (d, J=2.4 Hz, 1H), 7.97-7.98 (m, 1H), 7.72 (s, 1H), 7.54-7.65 (m, 3H), 7.51 (dd, J=8.8 Hz, 1.6 Hz, 1H), 7.46 (dd, J=10.0 Hz, 2.0 Hz, 1H), 7.29 (dd, J=8.4 Hz, 2.0 Hz, 1H), 6.87-7.02 (m, 2H), 6.67 (d, J=8.0 Hz, 1H), 6.47 (t, J=2.4 Hz, 1H), 5.38 (s, 2H), 5.08-5.14 (m, 1H), 4.68-4.73 (m, 1H), 4.56-4.61 (m, 1H), 4.44-4.49 (m, 1H), 4.35-4.41 (m, 1H), 3.90 (d, J=13.6 Hz, 1H), 3.76 (d, J=13.6 Hz, 1H), 2.95-3.05 (m, 1H), 2.85-2.88 (m, 1H), 2.57-2.71 (m, 2H), 2.39-2.49 (m, 1H), 2.12-2.28 (m, 2H), 1.62-1.82 (m, 4H).

### Example 56: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2-(1H-imidazol-1-yl)propanamide (Compound 195a)

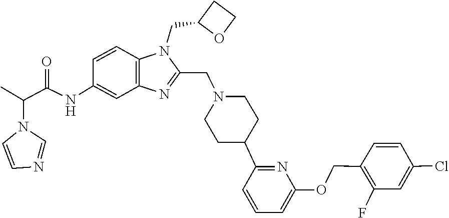 [SMILES: CC(C(=O)Nc1ccc2c(c1)nc(CN1CCC(c3cccc(OCc4ccc(Cl)cc4F)n3)CC1)n2C[C@@H]1CCO1)n1ccnc1]

The synthesis of Compound 195a is similar to that of Compound 102a except 2-(1H-imidazol-1-yl)propanoic acid is used instead of propionic acid. Compound 195a (11.29 mg, yield: 18%) is finally obtained as a white solid.

MS Calcd.: 657.3; MS Found: 658.3 [M+H]⁺.

¹H NMR (400 MHz, DMSO-d₆) δ 10.26 (s, 1H), 7.89 (d, J=1.6 Hz, 1H), 7.76 (s, 1H), 7.63 (dd, J=8.0 Hz, 7.2 Hz, 1H), 7.54-7.58 (m, 2H), 7.46 (dd, J=10.0 Hz, 2.0 Hz, 1H), 7.35 (dd, J=8.8 Hz, 1.6 Hz, 1H), 7.25-7.31 (m, 2H), 6.91 (s, 1H), 6.87 (d, J=7.2 Hz, 1H), 6.67 (d, J=8.0 Hz, 1H), 5.37 (s, 2H), 5.06-5.12 (m, 2H), 4.66-4.71 (m, 1H), 4.52-4.59 (m, 1H), 4.42-4.49 (m, 1H), 4.33-4.39 (m, 1H), 3.87 (d, J=13.2 Hz, 1H), 3.73 (d, J=13.6 Hz, 1H), 2.96-3.00 (m, 1H), 2.80-2.87 (m, 1H), 2.55-2.70 (m, 2H), 2.38-2.44 (m, 1H), 2.11-2.24 (m, 2H), 1.63-1.82 (m, 7H).

### Example 57: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3-methoxyoxane-3-carboxamide (Compound 196a)

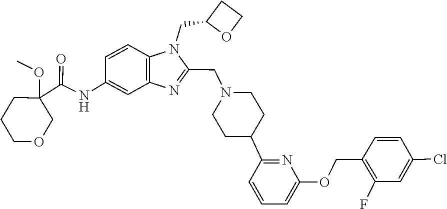 [SMILES: COC1(C(=O)Nc2ccc3c(c2)nc(CN2CCC(c4cccc(OCc5ccc(Cl)cc5F)n4)CC2)n3C[C@@H]2CCO2)CCCOC1]

The synthesis of Compound 196a is similar to that of Compound 102a except 3-methoxytetrahydro-2H-pyran-3-carboxylic acid is used instead of propionic acid. Compound 196a (25.44 mg, yield: 40%) is finally obtained as a white solid.

MS Calcd.: 677.3; MS Found: 678.4 [M+H]⁺.

¹H NMR (400 MHz, DMSO-d₆) δ 9.79 (s, 1H), 7.95 (s, 1H), 7.61-7.65 (m, 1H), 7.55-7.59 (m, 1H), 7.50-7.54 (m, 2H), 7.47 (dd, J=10.4 Hz, 2.0 Hz, 1H), 7.30 (dd, J=8.0 Hz, 0.8 Hz, 1H), 6.88 (d, J=7.2 Hz, 1H), 6.68 (d, J=8.0 Hz, 1H), 5.38 (s, 2H), 5.07-5.13 (m, 1H), 4.64-4.71 (m, 1H), 4.54-4.60 (m, 1H), 4.42-4.49 (m, 1H), 4.33-4.39 (m, 1H), 3.95 (d, J=12.0 Hz, 1H), 3.88 (d, J=13.6 Hz, 1H), 3.72-3.81 (m, 2H), 3.66 (d, J=12.4 Hz, 1H), 3.38-3.44 (m, 1H), 3.25 (s, 3H), 2.95-2.99 (m, 1H), 2.84-2.90 (m, 1H), 2.55-2.74 (m, 2H), 2.34-2.48 (m, 1H), 2.13-2.24 (m, 2H), 1.97-2.02 (m, 2H), 1.64-1.82 (m, 5H), 1.48-1.52 (m, 1H).

### Example 58: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-6-oxaspiro[3.5]nonane-7-carboxamide (Compound 197a)

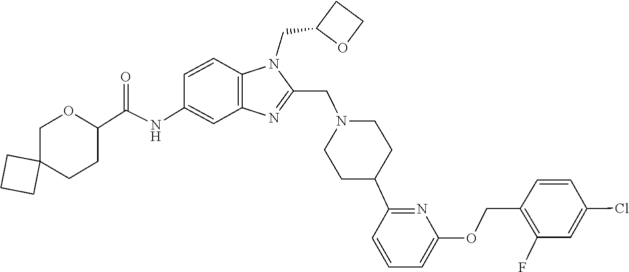 [SMILES: O=C(Nc1ccc2c(c1)nc(CN1CCC(c3cccc(OCc4ccc(Cl)cc4F)n3)CC1)n2C[C@@H]1CCO1)C1CCC2(CCC2)CO1]

The synthesis of Compound 197a is similar to that of Compound 102a except 6-oxaspiro[3.5]nonane-7-carboxylic acid is used instead of propionic acid. Compound 197a (20.30 mg, yield: 31.7%) is finally obtained as a white solid.

MS Calcd.: 687.3; MS Found: 688.4 [M+H]⁺.

¹H NMR (400 MHz, DMSO-d₆) δ 9.44 (s, 1H), 7.94-7.96 (m, 1H), 7.63 (t, J=8.0 Hz, 1H), 7.51-7.58 (m, 2H), 7.43-7.49 (m, 2H), 7.29 (dd, J=8.0 Hz, 1.6 Hz, 1H), 6.87 (d, J=7.2 Hz, 1H), 6.67 (d, J=8.0 Hz, 1H), 5.37 (s, 2H), 5.07-5.13 (m, 1H), 4.65-4.72 (m, 1H), 4.52-4.59 (m, 1H), 4.42-4.49 (m, 1H), 4.35-4.40 (m, 1H), 3.93 (dd, J=11.2 Hz, 1.6 Hz, 1H), 3.85-3.89 (m, 2H), 3.73 (d, J=13.6 Hz, 1H), 3.29-3.36 (m, 1H), 2.89-3.01 (m, 1H), 2.82-2.87 (m, 1H), 2.52-2.71 (m, 2H), 2.35-2.46 (m, 1H), 2.10-2.24 (m, 2H), 1.92-2.01 (m, 1H), 1.62-1.91 (m, 11H), 1.45-1.52 (m, 2H).

### Example 59: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3-methyl-2-oxabicyclo[2.2.2]octane-1-carboxamide (Compound 198a)

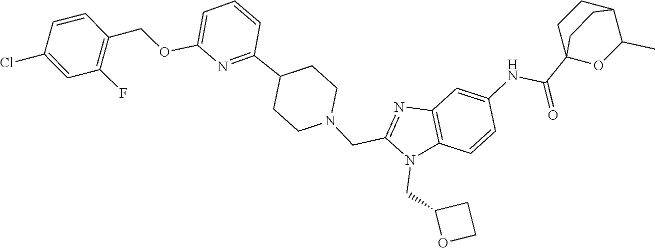 [SMILES: CC1OC2(C(=O)Nc3ccc4c(c3)nc(CN3CCC(c5cccc(OCc6ccc(Cl)cc6F)n5)CC3)n4C[C@@H]3CCO3)CCC1CC2]

The synthesis of Compound 198a is similar to that of Compound 102a except 3-methyl-2-oxabicyclo[2.2.2]octane-1-carboxylic acid is used instead of propionic acid. Compound 198a (30.28 mg, yield: 47%) is finally obtained as a white solid.

MS Calcd.: 687.3; MS Found: 688.3 [M+H]⁺.

¹H NMR (400 MHz, DMSO-d₆) δ 9.12 (s, 1H), 7.95 (s, 1H), 7.63 (t, J=8.0 Hz, 1H), 7.51-7.58 (m, 2H), 7.42-7.49 (m, 2H), 7.29 (dd, J=8.0 Hz, 1.6 Hz, 1H), 6.87 (d, J=7.2 Hz, 1H), 6.67 (d, J=8.0 Hz, 1H), 5.37 (s, 2H), 5.07-5.13 (m, 1H), 4.66-4.71 (m, 1H), 4.52-4.59 (m, 1H), 4.42-4.49 (m, 1H), 4.34-4.40 (m, 1H), 4.10-4.17 (m, 1H), 3.87 (d, J=13.2 Hz, 1H), 3.73 (d, J=13.2 Hz, 1H), 2.94-3.01 (m, 1H), 2.80-2.88 (m, 1H), 2.57-2.71 (m, 2H), 2.38-2.46 (m, 1H), 2.12-2.24 (m, 2H), 1.82-2.00 (m, 4H), 1.62-1.82 (m, 7H), 1.53-1.61 (m, 2H), 1.26 (d, J=6.4 Hz, 3H).

### Example 60: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2-cyclopropyloxolane-2-carboxamide (Compound 199a)

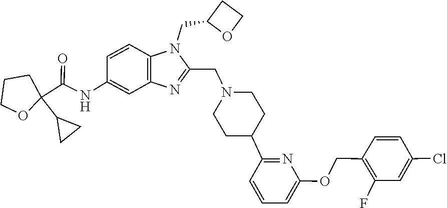 [SMILES: O=C(Nc1ccc2c(c1)nc(CN1CCC(c3cccc(OCc4ccc(Cl)cc4F)n3)CC1)n2C[C@@H]1CCO1)C1(C2CC2)CCCO1]

The synthesis of Compound 199a is similar to that of Compound 102a except 2-cyclopropyltetrahydrofuran-2-carboxylic acid is used instead of propionic acid. Compound 199a (18.33 mg, yield: 29%) is finally obtained as a white solid.

MS Calcd.: 673.3; MS Found: 674.4 [M+H]⁺.

¹H NMR (400 MHz, DMSO-d₆) δ 9.39 (s, 1H), 7.99 (s, 1H), 7.63 (t, J=8.0 Hz, 1H), 7.56 (t, J=8.4 Hz, 1H), 7.49 (br.s, 2H), 7.45 (dd, J=10.0 Hz, 2.0 Hz, 1H), 7.29 (dd, J=8.0 Hz, 2.0 Hz, 1H), 6.87 (d, J=7.2 Hz, 1H), 6.67 (d, J=8.0 Hz, 1H), 5.37 (s, 2H), 5.04-5.14 (m, 1H), 4.65-4.71 (m, 1H), 4.52-4.59 (m, 1H), 4.43-4.49 (m, 1H), 4.32-4.38 (m, 1H), 3.90-4.00 (m, 1H), 3.70-3.89 (m, 3H), 2.94-3.01 (m, 1H), 2.80-2.89 (m, 1H), 2.56-2.73 (m, 2H), 2.32-2.45 (m, 2H), 2.07-2.24 (m, 2H), 1.83-1.94 (m, 2H), 1.65-1.83 (m, 5H), 1.32-1.40 (m, 1H), 0.39-0.48 (m, 2H), 0.27-0.36 (m, 2H).

### Example 61: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}pyrimidine-5-carboxamide (Compound 200a)

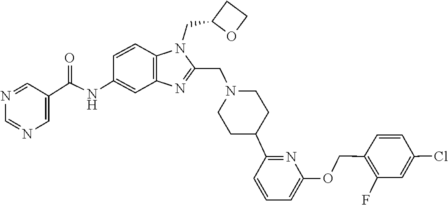 [SMILES: O=C(Nc1ccc2c(c1)nc(CN1CCC(c3cccc(OCc4ccc(Cl)cc4F)n3)CC1)n2C[C@@H]1CCO1)c1cncnc1]

The synthesis of Compound 200a is similar to that of Compound 102a except pyrimidine-5-carboxylic acid is used instead of propionic acid. Compound 200a (31.57 mg, yield: 53%) is finally obtained as a white solid.

MS Calcd.: 641.2; MS Found: 642.5 [M+H]⁺.

¹H NMR (400 MHz, DMSO-d₆) δ 10.57 (s, 1H), 9.37 (s, 1H), 9.30 (s, 2H), 8.05 (d, J=1.2 Hz, 1H), 7.59-7.65 (m, 2H), 7.52-7.57 (m, 2H), 7.46 (dd, J=10.0, 2.0 Hz, 1H), 7.30 (dd, J=8.4, 1.6 Hz, 1H), 6.88 (d, J=7.6 Hz, 1H), 6.68 (d, J=8.4 Hz, 1H), 5.38 (s, 2H), 5.09-5.16 (m, 1H), 4.69-4.75 (m, 1H), 4.55-4.62 (m, 1H), 4.44-4.51 (m, 1H), 4.37-4.42 (m, 1H), 3.91 (d, J=13.6 Hz, 1H), 3.76 (d, J=13.6 Hz, 1H), 2.95-3.03 (m, 1H), 2.84-2.91 (m, 1H), 2.55-2.74 (m, 2H), 2.40-2.49 (m, 1H), 2.12-2.27 (m, 2H), 1.64-1.83 (m, 4H).

### Example 62: (7S)—N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-5-oxaspiro[3.4]octane-7-carboxamide (Compound 201a)

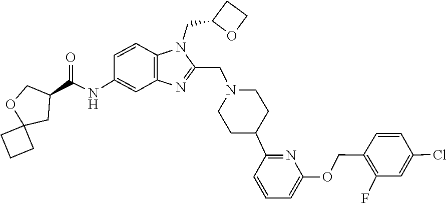 [SMILES: O=C(Nc1ccc2c(c1)nc(CN1CCC(c3cccc(OCc4ccc(Cl)cc4F)n3)CC1)n2C[C@@H]1CCO1)[C@@H]1COC2(CCC2)C1]

The synthesis of Compound 201a is similar to that of Compound 102a except (S)-5-oxaspiro[3.4]octane-7-carboxylic acid is used instead of propionic acid. Compound 201a (25.34 mg, yield: 40%) is finally obtained as a white solid.

MS Calcd.: 673.3; MS Found: 674.4 [M+H]⁺.

¹H NMR (400 MHz, DMSO-d₆) δ 9.94 (s, 1H), 7.90 (s, 1H), 7.63 (t, J=8.0 Hz, 1H), 7.51-7.59 (m, 2H), 7.46 (dd, J=10.0 Hz, 2.0 Hz, 1H), 7.34 (d, J=8.8 Hz, 1H), 7.29 (dd, J=8.0 Hz, 2.0 Hz, 1H), 6.87 (d, J=7.2 Hz, 1H), 6.67 (d, J=8.4 Hz, 1H), 5.37 (s, 2H), 5.06-5.12 (m, 1H), 4.63-4.72 (m, 1H), 4.53-4.58 (m, 1H), 4.42-4.50 (m, 1H), 4.34-4.40 (m, 1H), 3.95 (t, J=8.4 Hz, 1H), 3.87 (d, J=13.6 Hz, 1H), 3.68-3.79 (m, 2H), 3.17-3.29 (m, 1H), 2.95-3.00 (m, 1H), 2.82-2.89 (m, 1H), 2.54-2.71 (m, 2H), 2.38-2.46 (m, 1H), 1.93-2.32 (m, 8H), 1.49-1.81 (m, 6H).

### Example 63: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-1-methylpiperidine-3-carboxamide (Compound 202a)

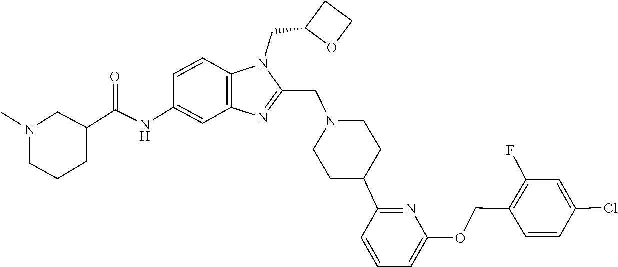 [SMILES: CN1CCCC(C(=O)Nc2ccc3c(c2)nc(CN2CCC(c4cccc(OCc5ccc(Cl)cc5F)n4)CC2)n3C[C@@H]2CCO2)C1]

The synthesis of Compound 202a is similar to that of Compound 102a except 1-methylpiperidine-3-carboxylic acid is used instead of propionic acid. Compound 202a (9.40 mg, yield: 15.7%) is finally obtained as a white solid.

MS Calcd.: 660.3; MS Found: 661.4 [M+H]⁺.

¹H NMR (400 MHz, DMSO-d₆) δ 9.88 (s, 1H), 7.90 (d, J=1.6 Hz, 1H), 7.63 (t, J=7.6 Hz, 1H), 7.56 (t, J=8.0 Hz, 1H), 7.50 (d, J=8.8 Hz, 1H), 7.46 (dd, J=10.0 Hz, 2.0 Hz, 1H), 7.34 (dd, J=8.8 Hz, 2.4 Hz, 1H), 7.30 (dd, J=8.4 Hz, 2.0 Hz, 1H), 6.87 (d, J=7.2 Hz, 1H), 6.67 (d, J=8.0 Hz, 1H), 5.37 (s, 2H), 5.05-5.12 (m, 1H), 4.62-4.71 (m, 1H), 4.52-4.57 (m, 1H), 4.42-4.49 (m, 1H), 4.34-4.39 (m, 1H), 3.87 (d, J=13.2 Hz, 1H), 3.73 (d, J=13.6 Hz, 1H), 2.95-3.00 (m, 1H), 2.82-2.88 (m, 2H), 2.54-2.72 (m, 4H), 2.34-2.45 (m, 1H), 2.10-2.23 (m, 5H), 1.99-2.05 (m, 1H), 1.62-1.89 (m, 7H), 1.35-1.56 (m, 2H).

### Example 64: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}piperidine-3-carboxamide (Compound 203a)

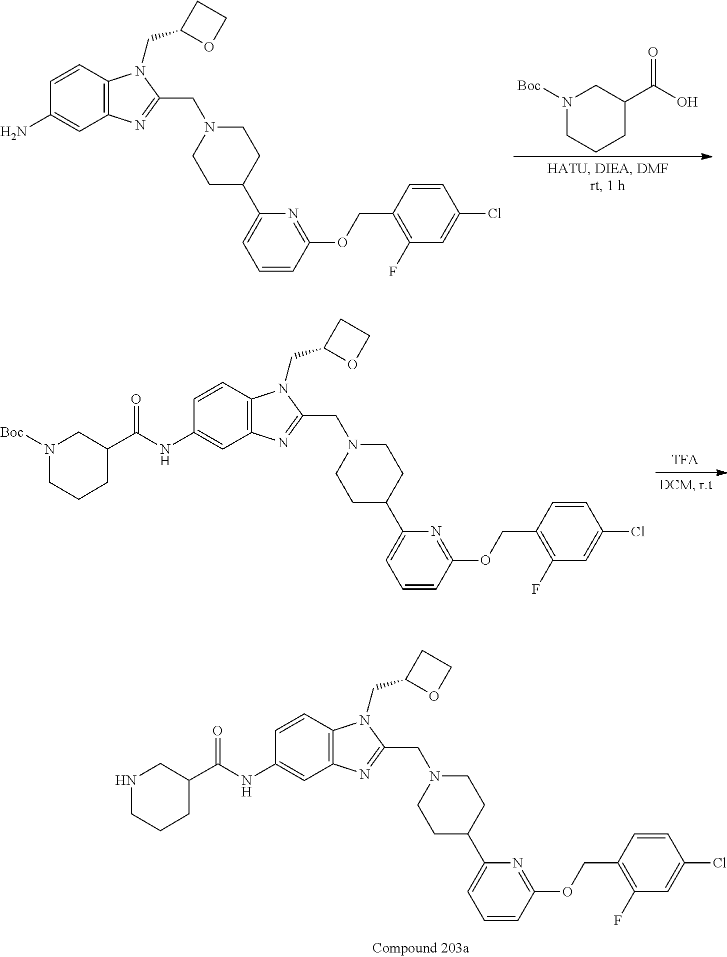 [SMILES: Nc1ccc2c(c1)nc(CN1CCC(c3cccc(OCc4ccc(Cl)cc4F)n3)CC1)n2C[C@@H]1CCO1 ; CC(C)(C)OC(=O)N1CCCC(C(=O)O)C1 ; CC(C)(C)OC(=O)N1CCCC(C(=O)Nc2ccc3c(c2)nc(CN2CCC(c4cccc(OCc5ccc(Cl)cc5F)n4)CC2)n3C[C@@H]2CCO2)C1 ; O=C(Nc1ccc2c(c1)nc(CN1CCC(c3cccc(OCc4ccc(Cl)cc4F)n3)CC1)n2C[C@@H]1CCO1)C1CCCNC1]

### Step A: tert-butyl 3-((2-((4-(6-((4-chloro-2-fluorobenzyl)oxy)pyridin-2-yl)piperidin-1-yl)methyl)-1-(((S)-oxetan-2-yl)methyl)-1H-benzo[d]imidazol-5-yl)carbamoyl)piperidine-1-carboxylate

To a solution of 1-(tert-butoxycarbonyl)piperidine-3-carboxylic acid (19 mg, 0.085 mmol) in DMF (1 mL) was added N,N-Diisopropylethylaamine (33 mg, 0.255 mmol), HATU (49 mg, 0.128 mmol), (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (50 mg, 0.093 mmol). The mixture was stirred at rt for 2 h. The resulting mixture was concentrated and the residue purified by column chromatography (DCM:MeOH=10:1) to afford tert-butyl 3-((2-((4-(6-((4-chloro-2-fluorobenzyl)oxy)pyridin-2-yl)piperidin-1-yl)methyl)-1-(((S)-oxetan-2-yl)methyl)-1H-benzo[d]imidazol-5-yl)carbamoyl)piperidine-1-carboxylate (86 mg, crude) as a light yellow oil.

MS Calcd.: 746.3; MS Found: 747.1 [M+H]⁺.

### Step B: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}piperidine-3-carboxamide (Compound 203a)

To a solution of tert-butyl 3-((2-((4-(6-((4-chloro-2-fluorobenzyl)oxy)pyridin-2-yl)piperidin-1-yl)methyl)-1-(((S)-oxetan-2-yl)methyl)-1H-benzo[d]imidazol-5-yl)carbamoyl)piperidine-1-carboxylate (86 mg, crude) in DCM (2 mL) was added TFA (0.768 g, 6.7 mmol) dropwise. The resulting mixture was stirred at rt for 2 h and then concentrated and the residue purified by prep-HPLC (0.1% NH₄HCO₃) to afford Compound 203a (7.3 mg, yield: 9.8%) as white solid.

MS Calcd.: 646.3; MS Found: 647.3 [M+H]⁺.

¹H NMR (400 MHz, CD₃OD): δ 9.87 (s, 1H), 7.90 (s, 1H), 7.63 (t, J=8.0 Hz, 1H), 7.56 (t, J=8.4 Hz, 1H), 7.50 (d, J=8.4 Hz, 1H), 7.44-7.47 (m, 1H), 7.33-7.35 (m, 1H), 7.28-7.31 (m, 1H), 6.87 (d, J=7.2 Hz, 1H), 6.67 (d, J=8.4 Hz, 1H), 5.37 (s, 2H), 5.08-5.12 (m, 1H), 4.62-4.69 (m, 1H), 4.53-4.57 (m, 1H), 4.43-4.49 (m, 1H), 4.32-4.40 (m, 1H), 3.86 (d, J=13.2 Hz, 1H), 3.73 (d, J=13.6 Hz, 1H), 2.92-3.04 (m, 2H), 2.80-2.90 (m, 2H), 2.57-2.69 (m, 3H), 2.39-2.45 (m, 3H), 2.10-2.23 (m, 2H), 1.55-1.89 (m, 7H), 1.35-1.44 (m, 1H).

### Example 65: N-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-b]pyridin-6-yl}acetamide (Compound 131a)

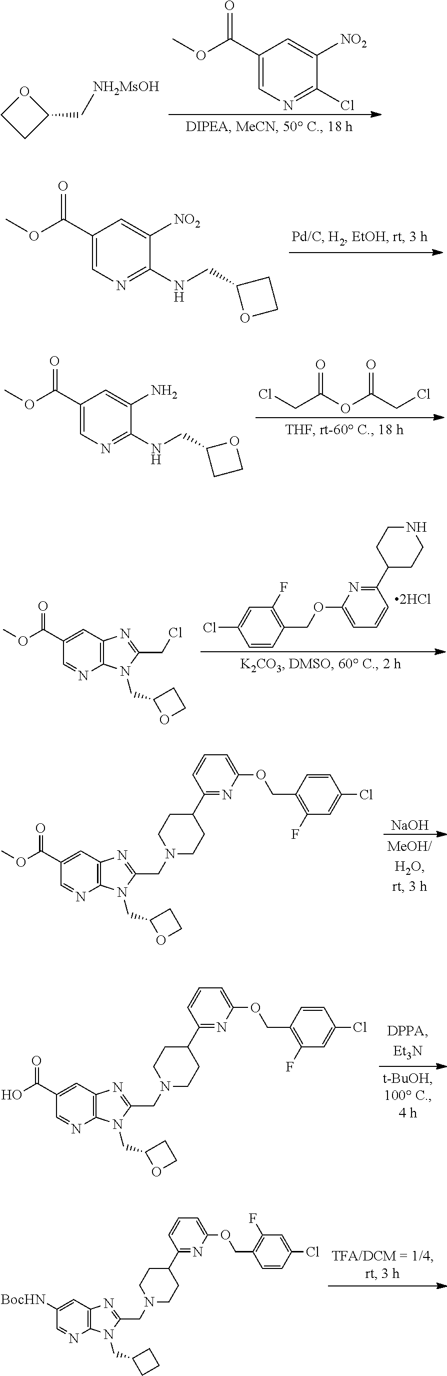

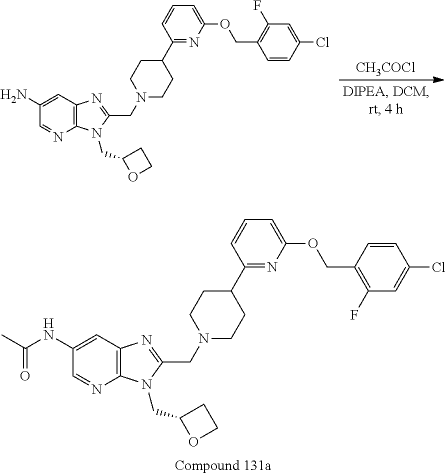

### Step A: methyl (S)-5-nitro-6-((oxetan-2-ylmethyl)amino)nicotinate

A reaction mixture of (S)-oxetan-2-ylmethanamine methanesulfonate (260 mg, 1.419 mmol), methyl 6-chloro-5-nitronicotinate (330 mg, 1.561 mmol) and DIPEA (916 mg, 7.098 mmol) in MeCN (6 ml) was stirred at 50° C. for 18 hours. Upon cooling down, the mixture was concentrated under vacuum. The residue was purified by silica gel column chromatography to furnish methyl (S)-5-nitro-6-((oxetan-2-ylmethyl)amino)nicotinate (310 mg, yield: 81.7%) as a yellow oil.

MS Calcd.: 267.1; MS Found: 268.4 [M+H]⁺.

### Step B: methyl (S)-5-amino-6-((oxetan-2-ylmethyl)amino)nicotinate

A mixture of methyl (S)-5-nitro-6-((oxetan-2-ylmethyl)amino)nicotinate (290 mg, 1.086 mmol) and Pd/C (20 mg, 10% w/w) in EtOH (6 ml) was stirred at room temperature under hydrogen atomsphere for 3 hours. The mixture was filtered and the solid was washed with hot EtOH (5×1 mL). The filtrate was concentrated under vacuum to furnish methyl (S)-5-amino-6-((oxetan-2-ylmethyl)amino)nicotinate (230 mg, yield: 89.3%) as an orange solid.

MS Calcd.: 237.1; MS Found: 238.2 [M+H]⁺.

### Step C: methyl (S)-2-(chloromethyl)-3-(oxetan-2-ylmethyl)-3H-imidazo[4,5-b]pyridine-6-carboxylate

To a stirred mixture of methyl (S)-5-amino-6-((oxetan-2-ylmethyl)amino)nicotinate (210 mg, 0.886 mmol) in dry THE (15 ml) was added a solution of 2-chloroacetic anhydride (215 mg, 1.263 mmol) in dry THE (5 ml) dropwise at room temperature. The reaction mixture was stirred at room temperature for 2 hours and then at 60° C. for 18 h. Upon cooling down, the reaction mixture was concentrated under vacuum. The residue was purified by silica gel column chromatography to furnish methyl (S)-2-(chloromethyl)-3-(oxetan-2-ylmethyl)-3H-imidazo[4,5-b]pyridine-6-carboxylate (130 mg, yield: 49.7%) as a white solid.

MS Calcd.: 295.1; MS Found: 296.0 [M+H]⁺.

### Step D methyl (S)-2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridine-6-carboxylate

A mixture of methyl (S)-2-(chloromethyl)-3-(oxetan-2-ylmethyl)-3H-imidazo[4,5-b]pyridine-6-carboxylate (130 mg, 0.441 mmol), 2-((4-chloro-2-fluorobenzyl)oxy)-6-(piperidin-4-yl)pyridine dihydrochloride (173 mg, 0.441 mmol) and K₂CO₃ (183 mg, 1.323 mmol) in DMSO (5 ml) was at 60° C. for 2 h. Upon cooling down, the mixture was filtered and the filtrate was purified by column chromatography to furnish methyl (S)-2-((4-(6-((4-chloro-2-fluorobenzyl)oxy)pyridine-2-yl)piperidin-1-yl)methyl)-3-(oxetan-2-ylmethyl)-3H-imidazo[4,5-b]pyridine-6-carboxylate (150 mg, yield: 58.7%) as a white solid.

MS Calcd.: 579.2; MS Found: 580.3 [M+H]⁺.

### Step E: (S)-2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridine-6-carboxylic acid

A mixture of methyl (S)-2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridine-6-carboxylate (150 mg, 0.259 mmol), NaOH (31 mg, 0.777 mmol) in MeOH (4 mL) and H₂O (2 mL) was stirred at room temperature for 3 hours. The reaction mixture was diluted with EA (30 mL), and pH value of aqueous layer was adjusted to 7 with 1N HCl at 0° C. The layers were separated and the aqueous layer was extracted with EA. The combined organic layers were dried over anhydrous Na₂SO₄, concentrated under vacuum to furnish (S)-2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridine-6-carboxylic acid (150 mg, crude) as a crude white solid.

MS Calcd.: 565.2; MS Found: 566.3 [M+H]⁺.

### Step F tert-butyl (S)-(2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridin-6-yl)carbamate

A mixture of (S)-2-((4-(6-((4-chloro-2-fluorobenzyl)oxy)pyridin-2-yl)piperidin-1-yl)methyl-3-(oxetan-2-ylmethyl)-3H-imidazo[4,5-b]pyridine-6-carboxylic acid (150 mg, 0.265 mmol), DPPA (109 mg, 0.398 mmol), Et₃N (81 mg, 0.796 mmol) in t-BuOH (3 mL) was stirred at 100° C. for 4 hours under Ar atmosphere. Upon cooling down, the reaction mixture was concentrated under vacuum. The residue was purified by Prep-TLC (DCM/MeOH=15/1) to furnish tert-butyl (S)-(2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridin-6-yl)carbamate (25 mg, yield: 14.8%) as white solid.

MS Calcd.: 636.3; MS Found: 637.3 [M+H]⁺.

### Step G: (S)-2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridin-6-amine

A mixture of tert-butyl (S)-(2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridin-6-yl)carbamate (70 mg, 0.012 mmol) in TFA (0.5 mL) and DCM (2 ml) was stirred at room temperature for 3 hours. The reaction mixture was diluted with EA (30 mL) and pH value of aqueous layer was adjusted to 7 with 1N HCl at 0° C. The layers were separated and the aqueous layer was extracted with EA. The combined organic layers were dried over anhydrous Na₂SO₄, concentrated under vacuum to furnish (S)-2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridin-6-amine (20 mg, crude) as a yellow oil.

MS Calcd.: 536.2; MS Found: 537.8 [M+H]⁺.

### Step H: N-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-b]pyridin-6-yl}acetamide (Compound 131a)

A mixture of (S)-2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridin-6-amine (20 mg, 0.037 mmol), CH₃COCl (5.8 mg, 0.074 mmol), DIPEA (0.1 mL) in DCM (2 ml) was stirred at room temperature for 4 hrs. The mixture was concentrated under vacuum. The residue was purified by prep-HPLC directly to give Compound 131a (6.3 mg, yield: 29.3%) as a white solid.

MS Calcd.: 578.2; MS Found: 579.2 [M+H]⁺.

¹H NMR (400 MHz, METHANOL-D4) δ 8.38 (s, 1H), 8.27 (s, 1H), 7.50-7.38 (m, 2H), 7.13-7.07 (m, 2H), 6.73 (d, J=12.0 Hz, 1H), 6.54 (d, J=8.0 Hz, 1H), 5.32 (s, 2H), 5.26-5.15 (m, 1H), 4.60-4.45 (m, 4H), 4.35-4.30 (m, 1H), 3.92-3.85 (m, 2H), 2.91-2.86 (m, 2H), 2.69-2.60 (m, 1H), 2.60-2.43 (m, 2H), 2.25-2.15 (m, 2H), 2.09 (s, 3H), 1.82-1.60 (m, 4H).

### Example 66: N-{2-[(4-{6-[(4-chloro-2-fluorophenyl)methoxy]pyridin-2-yl}-1,2,3,6-tetrahydropyridin-1-yl)methyl]-1-{[(2S)-oxetan-2-yl]methyl}-1H-1,3-benzodiazol-5-yl}acetamide (Compound 135a)

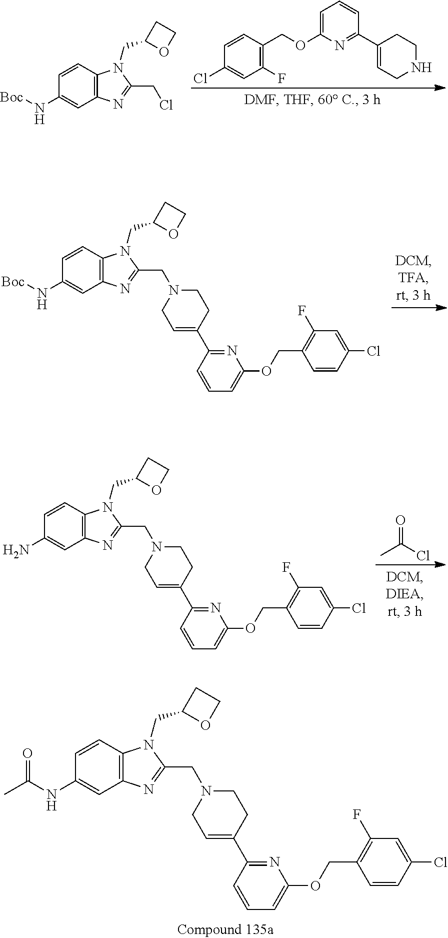

### Step A: tert-butyl (S)-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)carbamate

A mixture of tert-butyl (S)-(2-(chloromethyl)-1-(oxetan-2-ylmethyl)-1H-benzo[d]imidazol-5-yl)carbamate (50 mg, 0.14 mmol), 6-((4-chloro-2-fluorobenzyl)oxy)-1′,2′,3′,6′-tetrahydro-2,4′-bipyridine (45 mg) and K₂CO₃ (58 mg, 0.42 mmol) in DMF (1 ml) was stirred at 60° C. for 3 hours. The mixture was diluted with EtOAc (10 ml) and washed with water and brine. The organic layer was separated and dried over sodium sulfate, filtered and concentrated in vacuum. The residue was purified by column chromatography to give tert-butyl (S)-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)carbamate (60 mg, yield: 67.0%).

MS Calcd.: 633.2; MS Found: 634.0 [M+H]⁺.

### Step B: (S)-2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-amine

To a solution of tert-butyl (S)-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl) carbamate (60 mg, 0.09 mmol) in DCM (1 mL) was added TFA (0.4 mL). The resulting mixture was stirred at room temperature for 3 hours. The solvent was removed under vacuum to obtain (S)-2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-amine (50 mg, crude) as brown oil. The crude product was directly used for next step without further purification.

MS Calcd.: 533.2; MS Found: 534.4 [M+H]⁺.

### Step C: N-{2-[(4-{6-[(4-chloro-2-fluorophenyl)methoxy]pyridin-2-yl}-1,2,3,6-tetrahydropyridin-1-yl)methyl]-1-{[(2S)-oxetan-2-yl]methyl}-1H-1,3-benzodiazol-5-yl}acetamide (Compound 135a)

To a solution of (S)-2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-amine (50 mg, crude) and DIEA (50 mg, 0.09 mmol) in DCM (1 mL) was added acetyl chloride (9 mg, 0.10 mmol). The reaction was stirred at room temperature for 3 hours. After the reaction was completed, the solvent was removed in vacuo. The residue was purified by Prep-HPLC to give Compound 135a (10 mg, yield: 19%) as white solid.

MS Calcd.: 575.2; MS Found: 576.1 [M+H]⁺.

¹H NMR (400 MHz, DMSO-d₆): δ 9.87 (s, 1H), 7.91 (d, J=1.6 Hz, 1H), 7.68 (t, J=7.6 Hz, 1H), 7.55 (t, J=8.0 Hz, 1H), 7.50 (d, J=8.4 Hz, 1H), 7.46 (dd, J=10.0 Hz, J=2.4 Hz, 1H), 7.29-7.34 (m, 2H), 7.08 (d, J=7.6 Hz, 1H), 6.71-6.74 (m, 2H), 5.40 (s, 2H), 5.01-5.07 (m, 1H), 4.64-4.69 (m, 1H), 4.50-4.56 (m, 1H), 4.42-4.48 (m, 1H), 4.33-4.38 (m, 1H), 3.99 (d, J=13.2 Hz, 1H), 3.86 (d, J=13.2 Hz, 1H), 3.16-3.27 (m, 4H), 2.71-2.74 (m, 2H), 2.55-2.65 (m, 1H), 2.35-2.42 (m, 1H), 2.05 (s, 3H).

### Example 67: N-[2-({4-[6-(benzyloxy)pyridin-2-yl]piperidin-1-yl}methyl)-1-{[(2S)-oxetan-2-yl]methyl}-1H-1,3-benzodiazol-5-yl]pyridine-3-carboxamide (Compound 204a)

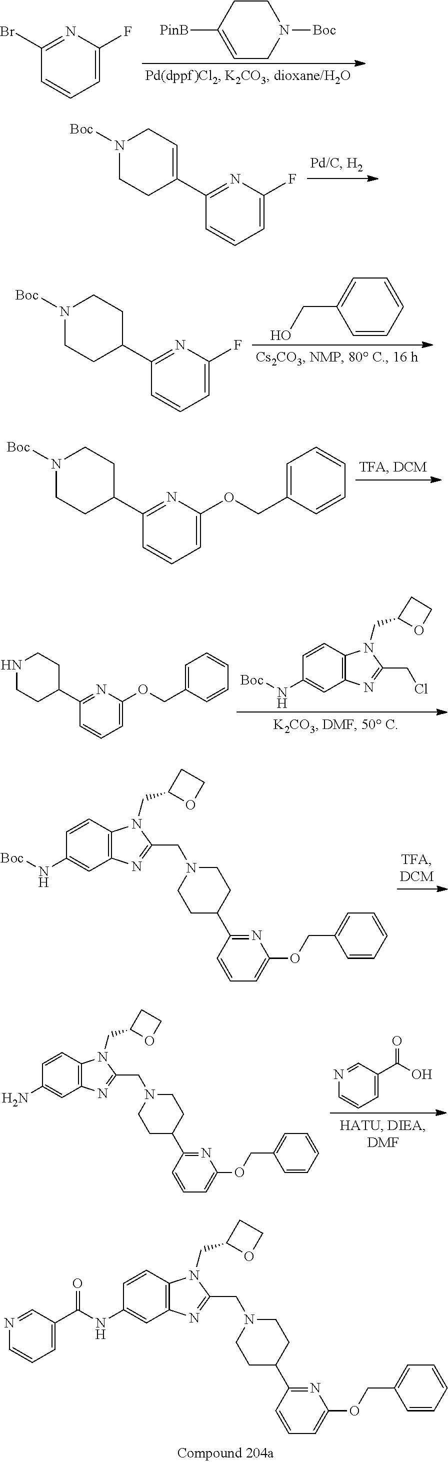

### Step A: tert-butyl 6-fluoro-3′,6′-dihydro-[2,4′-bipyridine]-1′(2′H)-carboxylate

To a solution of 2-bromo-6-fluoropyridine (500 mg, 2.9 mmol), tert-butyl 4-(4,4,5,5-tetramethyl-1,3,2-dioxaborolan-2-yl)-3,6-dihydropyridine-1(2H)-carboxylate (1.06 g, 3.5 mmol), Pd(dppf)Cl2 (116 mg, 0.15 mmol) and K₂CO₃ (1.18 g, 8.7 mmol) in dioxane (5 mL) and H₂O (0.5 mL) was degassed and charged with N₂ three times. The mixture was stirred at 90° C. for 3 hours. After the reaction was completed, the mixture was extracted with ethyl acetate (30 mL×3). The combined organic layer was washed with brine (30 mL×2), dried over sodium sulfate, filtered and concentrated in vacuum. The residue was purified by column chromatography to give tert-butyl 6-fluoro-3′,6′-dihydro-[2,4′-bipyridine]-1′(2′H)-carboxylate (700 mg, 88% yield) as a white solid.

MS Calcd.: 278.1; MS Found: 223.0 [M+H-56]⁺.

### Step B: tert-butyl 4-(6-fluoropyridin-2-yl)piperidine-1-carboxylate

To a solution of tert-butyl 6-fluoro-3′,6′-dihydro-[2,4′-bipyridine]-1′(2′H)-carboxylate (800 mg, 2.88 mmol) in MeOH was added Pd/C (wet, 10%, 80 mg). The mixture was stirred at room temperature under H₂ for 3 hours. The reaction was filtered and concentrated to give tert-butyl 4-(6-fluoropyridin-2-yl)piperidine-1-carboxylate (650 mg, yield: 92%) as a white solid.

MS Calcd.: 280.2; MS Found: 224.9 [M+H-56]⁺.

### Step C: tert-butyl 4-(6-(benzyloxy)pyridin-2-yl)piperidine-1-carboxylate

To a solution of tert-butyl 4-(6-fluoropyridin-2-yl)piperidine-1-carboxylate (650 mg, 2.3 mmol) and phenylmethanol (404 mg, 3.5 mmol) in NMP (10 mL) was added Cs₂CO₃ (1.6 g, 4.6 mmol) at room temperature. The reaction was stirred at 80° C. for 16 hours. After the reaction was completed, the mixture was diluted with water and extracted with ethyl acetate (100 mL×3). The organic layer was washed with brine (50 mL×2), dried over sodium sulfate, filtered and concentrated in vacuum. The residue was purified by column chromatography to give tert-butyl 4-(6-(benzyloxy)pyridin-2-yl)piperidine-1-carboxylate (100 mg, 12% yield) as white solid.

MS Calcd.: 368.2; MS Found: 369.2 [M+H]⁺.

### Step D: of 2-(benzyloxy)-6-(piperidin-4-yl)pyridine

To a solution of tert-butyl 4-(6-(benzyloxy)pyridin-2-yl)piperidine-1-carboxylate (100 mg, 0.27 mmol) in DCM (1 mL) was added TFA (1 mL). The reaction was stirred at room temperature for 1 hours. The reaction was concentrated under reduced pressure to afford 2-(benzyloxy)-6-(piperidin-4-yl)pyridine (100 mg, crude) as yellow oil.

MS Calcd.: 268.2; MS Found: 269.1 [M+H]⁺.

### Step E: tert-butyl (S)-(2-((4-(6-(benzyloxy)pyridin-2-yl)piperidin-1-yl)methyl)-1-(oxetan-2-ylmethyl)-1H-benzo[d]imidazol-5-yl)carbamate

A mixture of 2-(benzyloxy)-6-(piperidin-4-yl)pyridine (100 mg, 0.37 mmol) and tert-butyl (S)-(2-(chloromethyl)-1-(oxetan-2-ylmethyl)-1H-benzo[d]imidazol-5-yl)carbamate (130 mg, 0.37 mmol) in DMF (2 mL) was added K₂CO₃ (51 mg, 1.1 mmol) at room temperature. The reaction was stirred at 50° C. for 2 hours. After the reaction was completed, the mixture was diluted with water and extracted with ethyl acetate (100 mL×3). The organic layer was washed with brine (50 mL×2), dried over sodium sulfate, filtered and concentrated in vacuum. The residue was purified by column chromatography to give tert-butyl (S)-(2-((4-(6-(benzyloxy)pyridin-2-yl)piperidin-1-yl)methyl)-1-(oxetan-2-ylmethyl)-1H-benzo[d]imidazol-5-yl)carbamate (120 mg, 55% yield) as yellow solid.

### Step F: (S)-2-((4-(6-(benzyloxy)pyridin-2-yl)piperidin-1-yl)methyl)-1-(oxetan-2-ylmethyl)-1H-benzo[d]imidazol-5-amine

To a solution of tert-butyl (S)-(2-((4-(6-(benzyloxy)pyridin-2-yl)piperidin-1-yl)methyl)-1-(oxetan-2-ylmethyl)-11H-benzo[d]imidazol-5-yl)carbamate (120 mg, 0.2 mmol) in DCM (1 mL) was added TFA (1 mL). The reaction was stirred at room temperature for 1 hours. The reaction was concentrated under reduced pressure to afford (S)-2-((4-(6-(benzyloxy)pyridin-2-yl)piperidin-1-yl)methyl)-1-(oxetan-2-ylmethyl)-1H-benzo[d]imidazol-5-amine (120 mg, crude) as yellow oil.

MS Calcd.: 483.3; MS Found: 484.1 [M+H]⁺.

### Step G: N-[2-({4-[6-(benzyloxy)pyridin-2-yl]piperidin-1-yl}methyl)-1-{[(2S)-oxetan-2-yl]methyl}-1H-1,3-benzodiazol-5-yl]pyridine-3-carboxamide (Compound 204a)

To a solution of (S)-2-((4-(6-(benzyloxy)pyridin-2-yl)piperidin-1-yl)methyl)-1-(oxetan-2-ylmethyl)-1H-benzo[d]imidazol-5-amine (120 mg, 0.2 mmol) and nicotinic acid (25 mg, 0.2 mmol) in DMF (2 mL) was added DIEA (77 mg, 0.6 mmol) and HATU (94 mg, 0.24 mmol). The reaction was stirred at room temperature for 1 hours. The reaction mixture was purified directly by prep-HPLC to give Compound 204a (1.03 mg) as white solid.

MS Calcd.: 588.3; MS Found: 589.4 [M+H]⁺.

¹H NMR (400 MHz, DMSO-d₆): δ 9.11 (d, J=2.0 Hz, 1H), 8.72 (dd, J=4.8 Hz, 1.6 Hz, 1H), 8.35-8.38 (m, 1H), 8.07 (d, J=0.8 Hz, 1H), 7.53-7.63 (m, 4H), 7.39-7.43 (m, 2H), 7.30-7.34 (m, 2H), 7.23-7.27 (m, 1H), 6.79 (d, J=7.2 Hz, 1H), 6.61 (d, J=8.4 Hz, 1H), 5.36 (s, 2H), 5.23-5.29 (m, 1H), 4.78-4.84 (m, 1H), 4.64-4.69 (m, 1H), 4.57-4.62 (m, 1H), 4.40-4.47 (m, 1H), 3.96 (d, J=13.6 Hz, 1H), 3.85 (d, J=13.6 Hz, 1H), 3.02-3.08 (m, 1H), 2.89-2.95 (m, 1H), 2.73-2.81 (m, 1H), 2.59-2.66 (m, 1H), 2.47-2.54 (m, 1H), 2.21-2.33 (m, 2H), 1.82-1.91 (m, 4H).

### Example 68: (S)-2-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)isothiazolidine 1,1-dioxide (Compound 118a)

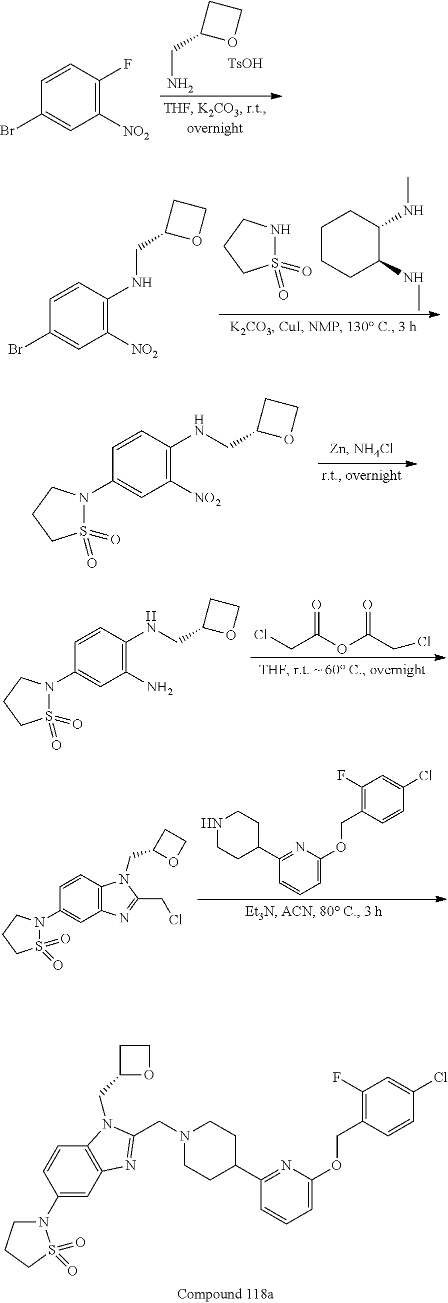

### Step A: (S)-4-bromo-2-nitro-N-(oxetan-2-ylmethyl)aniline

A mixture of 4-bromo-1-fluoro-2-nitrobenzene (2.00 g, 9.09 mmol), (S)-oxetan-2-ylmethanamine 4-methylbenzenesulfonate (1.81 g, 9.09 mmol), and K₂CO₃ (3.76 g, 27.3 mmol) in THF (20 mL) was degassed and refilled with N₂ for 3 times. The mixture was stirred at room temperature overnight under N₂ atmosphere. The reaction mixture was diluted with H₂O (200 mL), and extracted with EtOAc (200 mL*3). The organic layers were washed with brine, dried over Na₂SO₄, concentrated and purified by flash chromatography (PE/EtOAc=5/1) to afford (S)-4-bromo-2-nitro-N-(oxetan-2-ylmethyl)aniline as a yellow oil (2.80 g, 100% yield).

MS Calcd.: 286.0; MS Found: 286.8 [M+H]⁺.

### Step B: (S)-2-(3-nitro-4-((oxetan-2-ylmethyl)amino)phenyl)isothiazolidine 1,1-dioxide

A mixture of (S)-4-bromo-2-nitro-N-(oxetan-2-ylmethyl)aniline (400 mg, 1.39 mmol), isothiazolidine 1,1-dioxide (168 mg, 1.39 mmol), (1S,2S)—N¹,N²-dimethylcyclohexane-1,2-diamine (297 mg, 6.00 mmol), CuI (398 mg, 2.09 mmol) and K₂CO₃ (385 mg, 2.70 mmol) in NMP (4 mL) was degassed and purged with N₂ for 3 times. Then the mixture was stirred at 130° C. for 3 h under N₂ atmosphere. The reaction mixture was diluted with H₂O (10 mL), and extracted with DCM (10 mL*3). The organic layers were washed with brine, dried over Na₂SO₄, concentrated in vacuo and purified by flash chromatography (PE/EtOAc=1/1) to afford (S)-2-(3-nitro-4-((oxetan-2-ylmethyl)amino)phenyl)isothiazolidine 1,1-dioxide as a yellow oil (240 mg, 53% yield).

MS Calcd.: 327.0; MS Found: 328.2 [M+H]⁺.

### Step C: (S)-2-(3-amino-4-((oxetan-2-ylmethyl)amino)phenyl)isothiazolidine 1,1-dioxide

To a solution of (S)-2-(3-nitro-4-((oxetan-2-ylmethyl)amino)phenyl)isothiazolidine 1,1-dioxide (200 mg, 0.610 mmol) in MeOH (2 mL) were added Zn powder (397 mg, 6.10 mmol) and NH₄Cl (326 mg, 6.10 mmol).

The mixture was stirred at room temperature overnight under N₂ atmosphere. The reaction mixture was diluted with H₂O (50 mL), and extracted with EtOAc (50 mL*3).

The organic layers were dried over Na₂SO₄, concentrated in vacuo and purified by flash chromatography (PE/EtOAc=1/1) to afford (S)-2-(3-amino-4-((oxetan-2-ylmethyl)amino)phenyl)isothiazolidine 1,1-dioxide as a clear oil (180 mg, 99% yield).

MS Calcd.: 297.1; MS Found: 298.2 [M+H]⁺.

### Step D: (S)-2-(2-(chloromethyl)-1-(oxetan-2-ylmethyl)-1H-benzo[d]imidazol-5-yl)isothiazolidine 1,1-dioxide

A solution of (S)-2-(3-amino-4-((oxetan-2-ylmethyl)amino)phenyl)isothiazolidine 1,1-dioxide (220 mg, 0.740 mmol) and 2-chloroacetic anhydride (101 mg, 0.600 mmol) in THE (2 mL) was stirred at room temperature for 3 h and then heated to 60° C. overnight under N₂ atmosphere. The reaction mixture was concentrated in vacuo and purified by flash chromatography (PE/EtOAc=1/1) to afford (S)-2-(2-(chloromethyl)-1-(oxetan-2-ylmethyl)-1H-benzo[d]imidazol-5-yl)isothiazolidine 1,1-dioxide as a yellow oil (75.0 mg, 28.0% yield).

MS Calcd.: 355.1; MS Found: 356.2 [M+H]⁺.

### Step E: (S)-2-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)isothiazolidine 1,1-dioxide

A solution of (S)-2-(2-(chloromethyl)-1-(oxetan-2-ylmethyl)-1H-benzo[d]imidazol-5-yl)isothiazolidine 1,1-dioxide (75.0 mg, 0.210 mmol), 2-((4-chloro-2-fluorobenzyl)oxy)-6-(piperidin-4-yl)pyridine (74.4 mg, 0.230 mmol), and TEA (63.6 mg, 0.630 mmol) in ACN (2 mL) was degassed and purged with N₂ for 3 times. The mixture was stirred at 80° C. for 3 h under N₂ atmosphere. The mixture was purified by prep-HPLC (0.1% formic acid in water and acetonitrile) to afford Compound 118a (S)-2-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)isothiazolidine 1,1-dioxide as a white solid (37.8 mg, 28% yield).

¹H NMR (400 MHz, DMSO-d₆) δ 7.61-7.64 (m, 2H), 7.56 (t, J=8.4 Hz, 1H), 7.44-7.48 (m, 2H), 7.30 (dd, J=8.4, 2.0 Hz, 1H), 7.20 (dd, J=8.8, 2.0 Hz, 1H), 6.87 (d, J=7.2 Hz, 1H), 6.67 (d, J=8.0 Hz, 1H), 5.37 (s, 2H), 5.07-5.13 (m, 1H), 4.71 (dd, J=12.0, 6.8 Hz, 1H), 4.59 (dd, J=12.0, 2.8 Hz, 1H), 4.44-4.49 (m, 1H), 4.36-4.42 (m, 1H), 3.90 (d, J=13.2 Hz, 1H), 3.74-3.78 (m, 3H), 3.48 (t, J=7.6 Hz, 2H), 2.98 (d, J=10.8 Hz, 1H), 2.86 (d, J=10.8 Hz, 1H), 2.50-2.71 (m, 2H), 2.38-2.49 (m, 3H), 2.12-2.24 (m, 2H), 1.60-1.81 (m, 4H).

MS Calcd.: 639.2; MS Found: 640.2 [M+H]⁺.

### Example 69: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3,3-difluoropyrrolidine-1-carboxamide (Compound 205a)

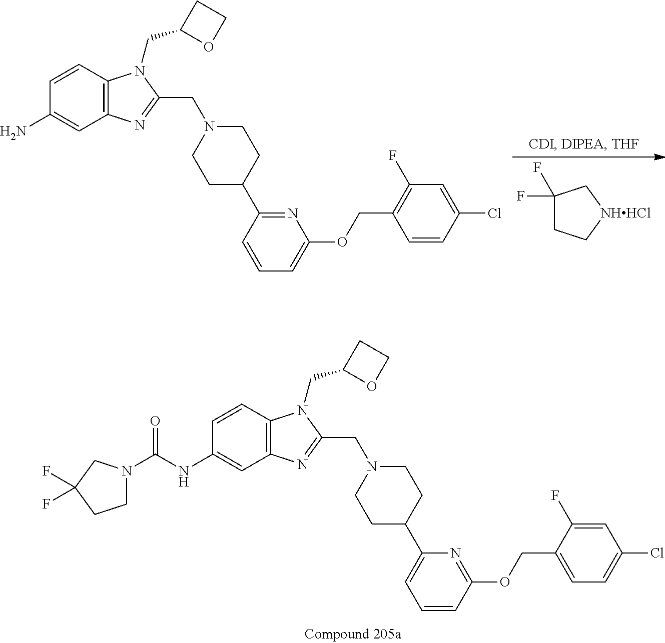

To a solution of (S)-2-((4-(6-((4-chloro-2-fluorobenzyl)oxy)pyridin-2-yl) piperidin-1-yl)methyl)-1-(oxetan-2-ylmethyl)-1H-benzo[d]imidazol-5-amine (40 mg, 0.07 mmol) in THE (3 mL) was added DIEA (96 mg, 0.7 mmol) and CDI (14.5 mg, 0.09 mmol). The mixture was stirred at room temperature for 1 hour, followed by addition of 3,3-difluoropyrrolidine hydrochloride (10 mg, 0.07 mmol). The reaction was stirred at room temperature for 4 hours, then filtered. The filtrate was purified by Prep-HPLC to give Compound 205a (11.2 mg, yield: 22.0%) as white solid.

MS Calcd.: 668.3; MS Found: 669.2 [M+H]⁺.

¹H NMR (400 MHz, methanol-d₄): δ7.71 (d, J=2.0 Hz, 1H), 7.61-7.47 (m, 3H), 7.36 (dd, J=2.0 Hz, J=8.8 Hz 1H), 7.24-7.19 (m, 2H), 6.84 (d, J=7.2 Hz, 1H), 6.65 (d, J=8.0 Hz, 1H), 5.43 (s, 2H), 5.30-5.26 (m, 1H), 4.72-4.65 (m, 1H), 4.50-4.43 (m, 1H), 3.97 (d, J=13.6 Hz, 1H), 3.89-3.81 (m, 3H), 3.74 (t, J=7.2 Hz, 2H), 3.05 (d, J=11.6 Hz, 1H), 2.94 (d, J=11.6 Hz, 1H), 2.85-2.47 (m, 5H), 2.33-2.21 (m, 3H), 2.06-2.04 (m, 1H), 1.88-1.80 (m, 4H).

¹⁹F-NMR (400 MHz, methanol-d₄): −117.59, −103.50

### Example 70: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3-fluoropyrrolidine-1-carboxamide (Compound 206a)

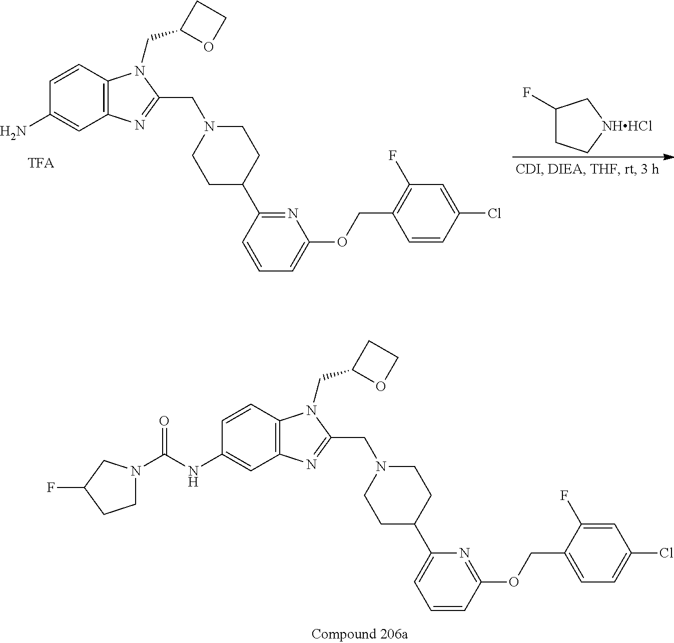

A mixture of (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (88 mg), DIEA (88 mg, 0.68 mmol) and CDI (22 mg, 0.136 mmol) in THF (5 ml) was stirred at room temperature for 1 hours. Then 3-fluoropyrrolidine hydrochloride (20 mg, 0.163 mmol) was added into the reaction mixture. The mixture was stirred at room temperature for 3 hours. The reaction mixture was filtered and the filtrate was purified by prep-HPLC directly to give Compound 206a (15.1 mg, yield: 17.1%) as white solid.

MS Calcd.: 650.3; MS Found: 651.2 [M+H]⁺.

¹H NMR (400 MHz, methanol-d₄) δ7.64 (d, J=1.6 Hz, 1H), 7.50-7.38 (m, 3H), 7.27 (dd, J=1.6 Hz, J=8.4 Hz 1H), 7.14-7.04 (m, 2H), 6.74 (d, J=7.6 Hz, 1H), 6.55 (d, J=8.4 Hz, 1H), 5.32 (s, 2H), 5.30-5.29 (m, 0.5H), 5.18-5.13 (m, 1.5H), 4.68 (dd, J=6.8 Hz, J=15.2 Hz 1H), 4.58-4.50 (m, 2H), 4.37-4.30 (m, 1H), 3.99 (d, J=14.0 Hz, 1H), 3.91 (d, J=14.0 Hz, 1H), 3.73-3.58 (m, 2.5H), 3.53-3.44 (m, 1.5H), 3.07 (d, J=10.8 Hz, 1H), 2.98 (d, J=11.6 Hz, 1H), 2.70-2.56 (m, 2H), 2.47-2.30 (m, 3H), 2.25-1.90 (m, 2H), 1.85-1.75 (m, 4H).

### Example 71: N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3-hydroxypyrrolidine-1-carboxamide (Compound 207a)

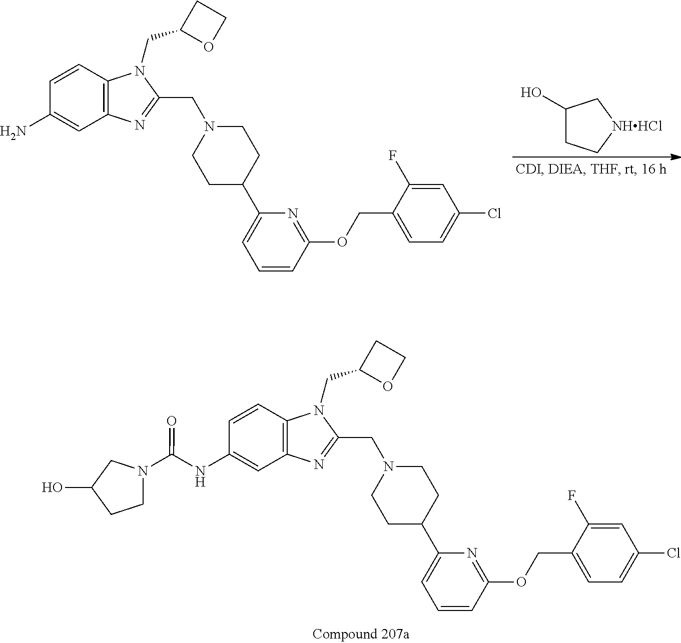

A mixture of (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (88 mg), DIEA (88 mg, 0.68 mmol) and CDI (22 mg, 0.136 mmol) in THF (5 ml) was stirred at room temperature for 1 hours. Then pyrrolidin-3-ol hydrochloride (20 mg, 0.163 mmol) was added into the reaction mixture. The mixture was stirred at room temperature for 16 hours. The reaction mixture was filtered and the filtrate was purified by prep-HPLC directly to give Compound 207a (8.9 mg, yield: 10.1%) as white solid.

MS Calcd.: 648.3; MS Found: 649.2 [M+H]⁺.

¹H NMR (400 MHz, methanol-d₄) δ 7.77 (d, J=1.6 Hz, 1H), 7.58 (t, J=7.6 Hz, 1H), 7.53-7.46 (m, 2H), 7.37 (dd, J=1.6 Hz, J=8.8 Hz 1H), 7.22-7.17 (m, 2H), 6.84 (d, J=7.6 Hz, 1H), 6.65 (d, J=8.4 Hz, 1H), 5.41 (s, 2H), 5.25-5.20 (m, 1H), 4.76 (dd, J=6.8 Hz, J=15.2 Hz 1H), 4.66-4.58 (m, 2H), 4.48-4.41 (m, 2H), 4.20 (dd, J=14.4 Hz, J=22.4 Hz, 2H), 3.63-3.57 (m, 3H), 3.47 (d, J=11.2 Hz, 1H), 3.30-3.24 (m, 2H), 2.81-2.73 (m, 2H), 2.70-2.61 (m, 2H), 2.56-2.49 (m, 1H), 2.11-2.03 (m, 1H), 2.01-1.94 (m, 5H).

### Example 72: 1-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-3-methylurea (Compound 145a)

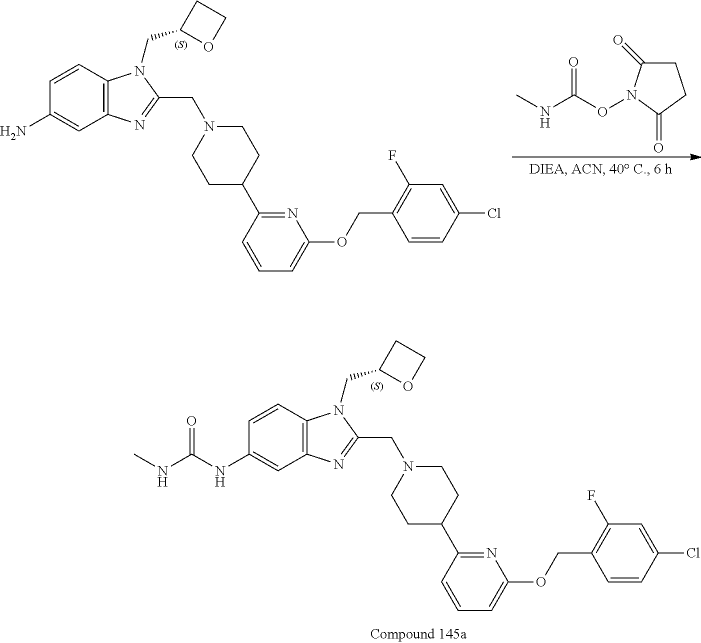

A mixture of (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (50 mg, 0.093 mmol), 2,5-dioxopyrrolidin-1-yl methylcarbamate (21 mg, 0.121 mmol) and DIEA (18 mg, 0.139 mmol) in acetonitrile (2 mL) was heated to 40° C. for 6 hours. The solvent was removed in vacuo. The residue was purified by Prep-HPLC to give Compound 145a (8.57 mg, yield: 15%) as a white solid.

MS Calcd.: 592.2; MS Found: 593.1 [M+H]⁺.

¹H NMR (400 MHz, DMSO-d₆): 8.36 (s, 1H), 7.67 (d, J=2.0 Hz, 1H), 7.63 (t, J=7.6 Hz, 1H), 7.56 (t, J=8.4 Hz, 1H), 7.43-7.47 (m, 2H), 7.29 (dd, J=8.4 Hz, 1.6 Hz, 1H), 7.13 (dd, J=8.8 Hz, 2.0 Hz, 1H), 6.87 (d, J=7.2 Hz, 1H), 6.67 (d, J=8.0 Hz, 1H), 5.90 (q, J=4.4 Hz, 1H), 5.38 (s, 2H), 5.06-5.12 (m, 1H), 4.62-4.68 (m, 1H), 4.51-4.55 (m, 1H), 4.44-4.49 (m, 1H), 4.35-4.40 (m, 1H), 3.86 (d, J=13.2 Hz, 1H), 3.71 (d, J=13.2 Hz, 1H), 2.95-2.99 (m, 1H), 2.80-2.90 (m, 1H), 2.57-2.71 (m, 5H), 2.38-2.47 (m, 1H), 2.07-2.23 (m, 2H), 1.62-1.82 (m, 4H).

### Example 73: methyl N-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}carbamate (Compound 146a)

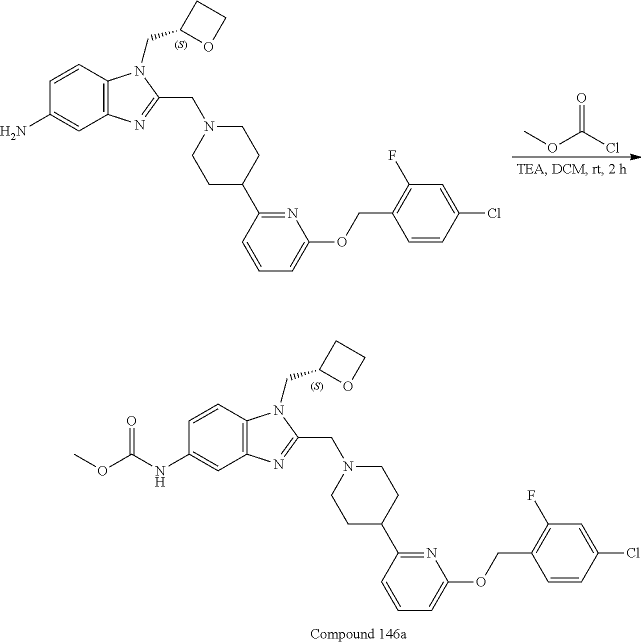

To a solution of (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (50 mg, 0.093 mmol) in DCM (2 mL) was added triethylamine (19 mg, 0.188 mmol) and methyl carbonochloridate (13 mg, 0.138 mmol). The resulting mixture was stirred at room temperature for 2 hours. The solvent was removed in vacuo. The residue was purified by Prep-HPLC to give Compound 146a (6.56 mg, yield: 11.8%) as a white solid.

MS Calcd.: 593.2; MS Found: 594.1 [M+H]⁺.

¹H NMR (400 MHz, DMSO-d₆): 9.52 (s, 1H), 7.74 (s, 1H), 7.63 (t, J=7.6 Hz, 1H), 7.56 (t, J=8.0 Hz, 1H), 7.51 (d, J=8.4 Hz, 1H), 7.46 (dd, J=10.0 Hz, 2.0 Hz, 1H), 7.30 (dd, J=8.0 Hz, 1.6 Hz, 1H), 7.20-7.25 (m, 1H), 6.88 (d, J=7.2 Hz, 1H), 6.68 (d, J=8.4 Hz, 1H), 5.38 (s, 2H), 5.05-5.12 (m, 1H), 4.64-4.70 (m, 1H), 4.52-4.60 (m, 1H), 4.42-4.49 (m, 1H), 4.34-4.39 (m, 1H), 3.74-3.92 (m, 2H), 3.66 (s, 3H), 2.82-3.04 (m, 2H), 2.55-2.71 (m, 2H), 2.39-2.47 (m, 1H), 2.10-2.35 (m, 2H), 1.64-1.86 (m, 4H).

### Example 74: 1-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}pyrrolidin-2-one (Compound 119a)

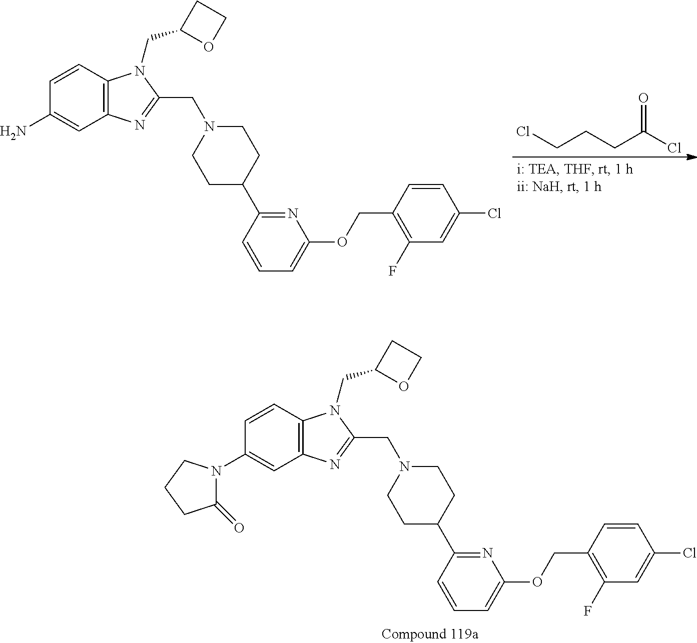 [SMILES: Nc1ccc2c(c1)nc(CN1CCC(c3cccc(OCc4ccc(Cl)cc4F)n3)CC1)n2C[C@@H]1CCO1 ; O=C(Cl)CCCCl ; O=C1CCCN1c1ccc2c(c1)nc(CN1CCC(c3cccc(OCc4ccc(Cl)cc4F)n3)CC1)n2C[C@@H]1CCO1]

A mixture of (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (50 mg, 0.09 mmol), 4-chlorobutanoyl chloride (15 mg, 1.1 mmol), TEA (27 mg, 0.27 mmol) in THF (2 mL) was stirred at room temperature for 1 h. Then NaH (5.4 mg, 0.14 mmol, 60% purity) was added, the reaction was stirred at room temperature for 1 h. The reaction was diluted with ethyl acetate and was washed with brine, dried over sodium sulfate, filtered and concentrated under reduced pressure. The residue was purified by prep-HPLC to give Compound 119a (28.2 mg, yield: 50%) as a white solid.

MS Calcd.: 603.2; MS Found: 604.5 [M+H]⁺.

¹H NMR (400 MHz, DMSO-d₆): δ 7.74 (d, J=1.6 Hz, 1H), 7.51-7.65 (m, 4H), 7.46 (dd, J=10.0 Hz, 2.0 Hz, 1H), 7.29 (dd, J=8.4 Hz, 2.0 Hz, 1H), 6.87 (d, J=7.2 Hz, 1H), 6.67 (d, J=8.0 Hz, 1H), 5.38 (s, 2H), 5.08-5.14 (m, 1H), 4.68-4.73 (m, 1H), 4.56-4.61 (m, 1H), 4.42-4.49 (m, 1H), 4.32-4.40 (m, 1H), 3.86-3.91 (m, 3H), 3.75 (d, J=13.2 Hz, 1H), 2.95-3.02 (m, 1H), 2.84-2.88 (m, 1H), 2.56-2.72 (m, 2H), 2.47-2.50 (m, 2H), 2.38-2.45 (m, 1H), 2.04-2.25 (m, 4H), 1.63-1.82 (m, 4H).

### Example 75: 3-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-1,3-oxazolidin-2-one (Compound 120a)

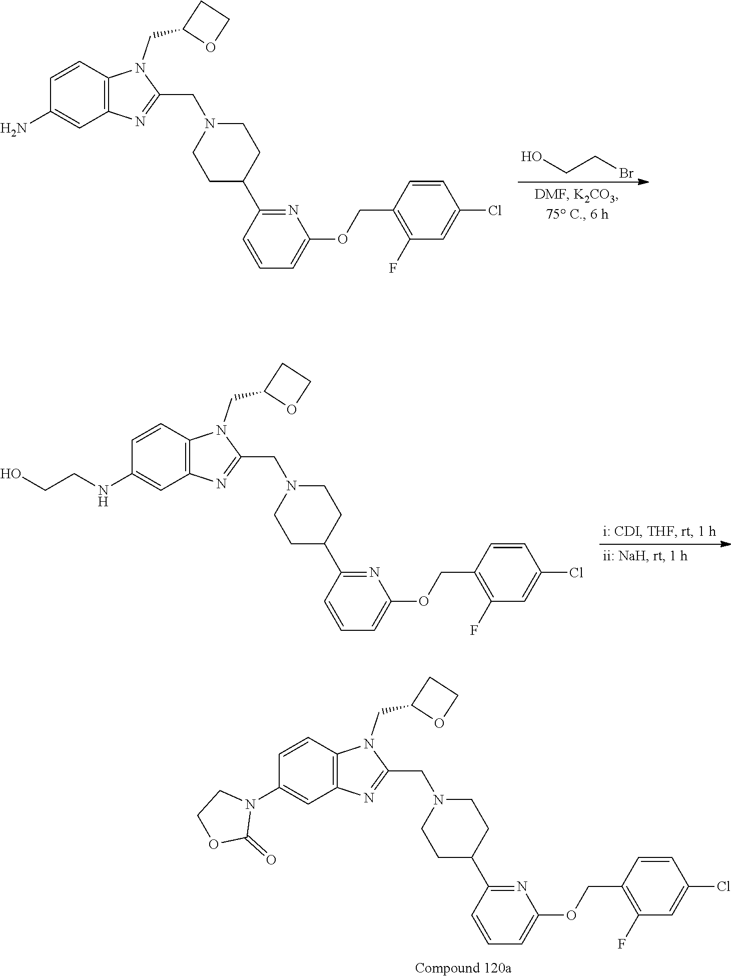

### Step A: (S)-2-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)amino)ethan-1-ol

A mixture of (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (50 mg, 0.093 mmol), 2-bromoethan-1-ol (18 mg, 0.14 mmol), K₂CO₃ (39 mg, 0.28 mmol) in DMF (2 mL) was stirred at 75° C. for 6 h under Ar. The mixture was cooled to room temperature and added H₂O, extracted with DCM (2×10 ml). The combined organic layer was dried over sodium sulfate, filtered and concentrated under reduced pressure. The residue was purified by column chromatography to give (S)-2-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)amino)ethan-1-ol (25 mg, yield: 46%) as a white solid.

MS Calcd.: 579.2; MS Found: 580.2 [M+H]⁺.

### Step B: 3-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-1,3-oxazolidin-2-one (Compound 120a)

A mixture of (S)-2-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)amino)ethan-1-ol (25 mg, 0.05 mmol), TEA (19 mg, 0.15 mmol), CDI (24 mg, 0.15 mmol) in THE (2 mL) was stirred at room temperature for 1 h. The mixture was cooled to 0° C. and NaH (12 mg, 0.3 mmol, 60% purity) was added. The mixture was stirred at room temperature for 1 h, quenched with MeOH. The resulting mixture was purified by Prep-HPLC to give Compound 120a (6.74 mg, yield: 22%) as a white solid (HCOOH salt).

MS Calcd.: 605.2; MS Found: 606.4 [M+H]⁺.

¹H NMR (400 MHz, DMSO-d₆): δ 8.14 (s, 1H), 7.71 (s, 1H), 7.62-7.66 (m, 2H), 7.57 (t, J=8.4 Hz, 1H), 7.49-7.52 (m, 1H), 7.46 (dd, J=10.0 Hz, 2.0 Hz, 1H), 7.29 (dd, J=8.4 Hz, 2.0 Hz, 1H), 6.88 (d, J=7.2 Hz, 1H), 6.68 (d, J=8.0 Hz, 1H), 5.38 (s, 2H), 5.04-5.13 (m, 1H), 4.69-4.76 (m, 1H), 4.54-4.62 (m, 1H), 4.40-4.50 (m, 3H), 4.34-4.39 (m, 1H), 4.09-4.14 (m, 2H), 3.77-4.01 (m, 2H), 2.80-3.12 (m, 2H), 2.60-2.73 (m, 2H), 2.10-2.45 (m, 3H), 1.73-1.86 (m, 4H).

### Example 76: 1-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2,3-dihydro-1H-imidazol-2-one (Compound 122a)

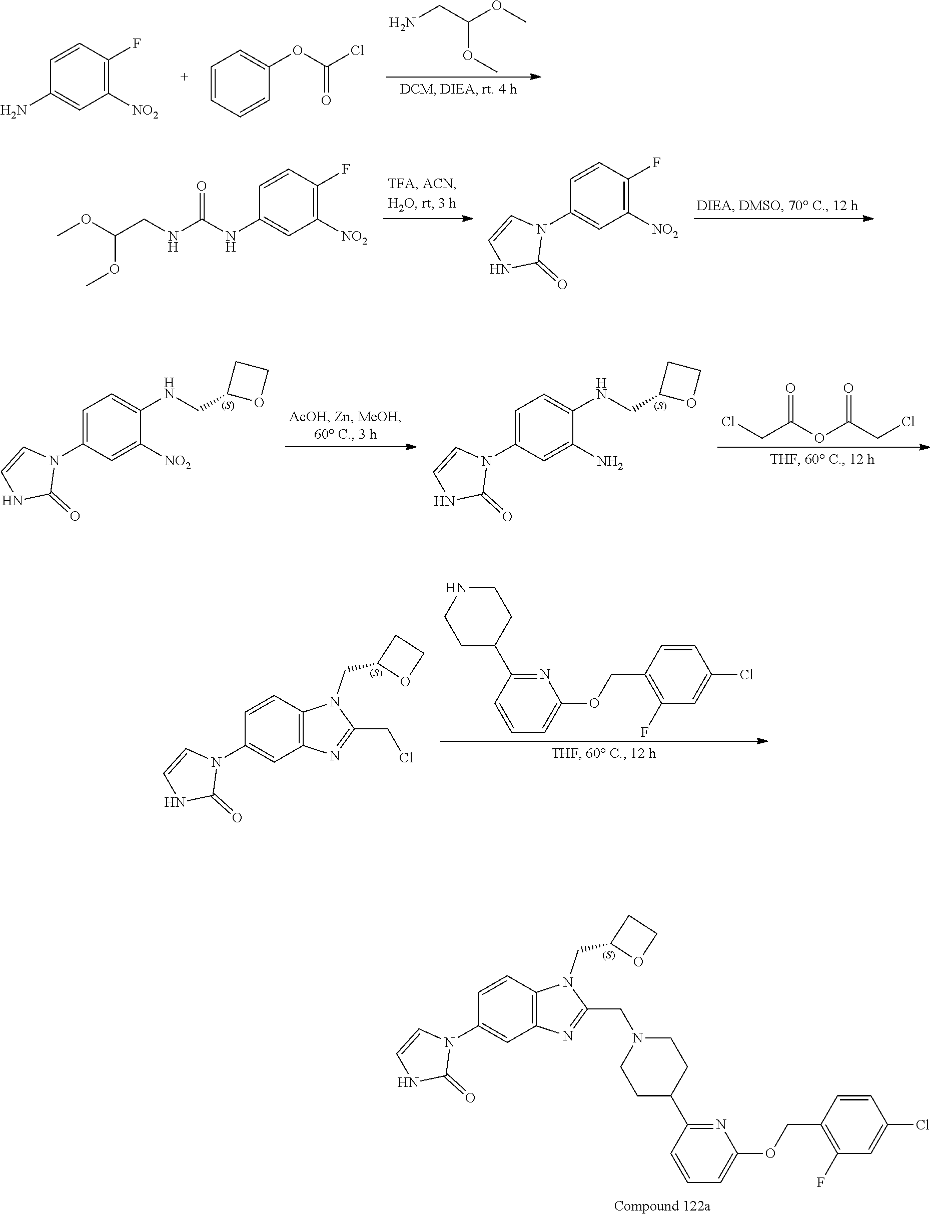

### Step A: 1-(2,2-dimethoxyethyl)-3-(4-fluoro-3-nitrophenyl)urea

To a solution of 4-fluoro-3-nitroaniline (1.0 g, 6.41 mmol) in DCM (15 mL) was added DIEA (2.48 g, 19.2 mmol) and phenyl carbonochloridate (1.1 g, 7.05 mmol) at room temperature. The reaction mixture was stirred at room temperature for 2 hours. Then 2,2-dimethoxyethan-1-amine (740 mg, 7.05 mmol) was added to the above reaction mixture. The resulting mixture was stirred at room temperature for 2 hours. After the reaction was completed, the reaction was quenched with water (200 mL) and extracted with ethyl acetate (100 mL×3). The organic layer was combined and washed with brine (100 mL×2), dried over sodium sulfate, filtered and concentrated in vacuum. The residue was purified by column chromatography to give 1-(2,2-dimethoxyethyl)-3-(4-fluoro-3-nitrophenyl)urea (530 mg, 29.4% yield) as a yellow solid.

MS Calcd.: 287.1; MS Found: 286.0 [M−H]⁻.

### Step B: 1-(4-fluoro-3-nitrophenyl)-1,3-dihydro-2H-imidazol-2-one

To a solution of 1-(2,2-dimethoxyethyl)-3-(4-fluoro-3-nitrophenyl)urea (530 mg, 1.84 mmol) in ACN (4 mL) and H₂O (4 mL) was added TFA (2 mL). The reaction was stirred at room temperature for 3 hours. After the reaction was completed, the reaction was quenched with water (20 mL) and extracted with ethyl acetate (20 mL×3). The organic layer was combined and washed with brine (100 mL×2), dried over sodium sulfate, filtered and concentrated in vacuum. The residue was purified by column chromatography to give 1-(4-fluoro-3-nitrophenyl)-1,3-dihydro-2H-imidazol-2-one (335 mg, 81% yield) as a yellow solid.

MS Calcd.: 223.0; MS Found: 224.0 [M+H]⁺.

### Step C: (S)-1-(3-nitro-4-((oxetan-2-ylmethyl)amino)phenyl)-1,3-dihydro-2H-imidazol-2-one

To a solution of 1-(4-fluoro-3-nitrophenyl)-1,3-dihydro-2H-imidazol-2-one (150 mg, 0.67 mmol) and (S)-oxetan-2-ylmethanamine MsOH salt (134 mg, 0.73 mmol) in DMSO (20 mL) was added DIEA (259 mg, 2.01 mmol) at room temperature.

The reaction was stirred at 70° C. for 12 hours. After the reaction was completed, the reaction was quenched with water (20 mL) and extracted with ethyl acetate (100 mL×3). The organic layer was combined and washed with brine (20 mL×2), dried over sodium sulfate, filtered and concentrated in vacuum. The residue was purified by column chromatography to give (S)-1-(3-nitro-4-((oxetan-2-ylmethyl)amino)phenyl)-1,3-dihydro-2H-imidazol-2-one (140 mg, 72% yield) as a red solid.

MS Calcd.: 290.1; MS Found: 291.2 [M+H]⁺.

### Step D: (S)-1-(3-amino-4-((oxetan-2-ylmethyl)amino)phenyl)-1,3-dihydro-2H-imidazol-2-one

To a solution of (S)-1-(3-nitro-4-((oxetan-2-ylmethyl)amino)phenyl)-1,3-dihydro-2H-imidazol-2-one (140 mg, 0.48 mmol) and Zn (312 mg, 4.8 mmol) in MeOH (3 mL) was added AcOH (1 mL). The reaction was stirred at 60° C. for 3 hours. After the reaction was completed, the reaction was filtered and the filtrate was quenched with water (20 mL) and extracted with ethyl acetate (20 mL×3). The organic layer was combined and washed with brine (20 mL×2), dried over sodium sulfate, filtered and concentrated in vacuum to give (S)-1-(3-amino-4-((oxetan-2-ylmethyl)amino)phenyl)-1,3-dihydro-2H-imidazol-2-one (85 mg, 68% yield) as a brown solid.

MS Calcd.: 260.1; MS Found: 261.0[M+H]⁺.

### Step E: (S)-1-(2-(chloromethyl)-1-(oxetan-2-ylmethyl)-1H-benzo[d]imidazol-5-yl)-1,3-dihydro-2H-imidazol-2-one

A mixture of (S)-1-(3-amino-4-((oxetan-2-ylmethyl)amino)phenyl)-1,3-dihydro-2H-imidazol-2-one (85 mg, 0.326 mmol) and 2-chloroacetic anhydride (67.1 mg, 0.392 mmol) in THE (1.5 ml) was stirred at 60° C. for 12 hours. The mixture was concentrated under reduced pressure and the residue purified by column chromatography to give (S)-1-(2-(chloromethyl)-1-(oxetan-2-ylmethyl)-1H-benzo[d]imidazol-5-yl)-1,3-dihydro-2H-imidazol-2-one (30 mg, yield: 29%) as a brown solid.

MS Calcd.: 318.1; MS Found: 319.0 [M+H]⁺.

### Step F: 1-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-2,3-dihydro-1H-imidazol-2-one (Compound 122a)

A mixture of (S)-1-(2-(chloromethyl)-1-(oxetan-2-ylmethyl)-1H-benzo[d]imidazol-5-yl)-1,3-dihydro-2H-imidazol-2-one (30 mg, 0.09 mmol), 2-((4-chloro-2-fluorobenzyl)oxy)-6-(piperidin-4-yl)pyridine (40 mg) and K₂CO₃ (39 mg, 0.28 mmol) in DMF (1 ml) was stirred at 60° C. for 3 hours. The mixture was purified by Prep-HPLC to give Compound 122a (4.9 mg, yield: 9.0%) as a white solid.

MS Calcd.: 602.2; MS Found: 603.4 [M+H]⁺.

¹H NMR (400 MHz, DMSO-d₆): 10.23 (s, 1H), 8.30 (br s, 1H), 7.81 (d, J=2.0 Hz, 1H), 7.61-7.67 (m, 1H), 7.56 (t, J=8.4 Hz, 1H), 7.51 (dd, J=8.8 Hz, J=2.0, 1H), 7.46 (dd, J=10.0 Hz, J=2.0 Hz, 1H), 7.29 (dd, J=8.4 Hz, J=1.6 Hz, 1H), 6.90-6.94 (m, 1H), 6.87 (d, J=7.2 Hz, 1H), 6.67 (d, J=8.0 Hz, 1H), 6.56 (t, J=2.8 Hz, 1H), 5.38 (s, 2H), 5.09-5.14 (m, 1H), 4.68-4.76 (m, 1H), 4.57-4.63 (m, 1H), 4.43-4.50 (m, 1H), 4.35-4.41 (m, 1H), 3.91 (d, J=13.2 Hz, 1H), 3.77 (d, J=13.2 Hz, 1H), 2.96-3.31 (m, 1H), 2.85-2.90 (m, 1H), 2.57-2.72 (m, 2H), 2.41-2.46 (m, 1H), 2.12-2.25 (m, 2H), 1.65-1.82 (m, 4H).

### Example 77: 1-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}imidazolidin-2-one (Compound 123a)

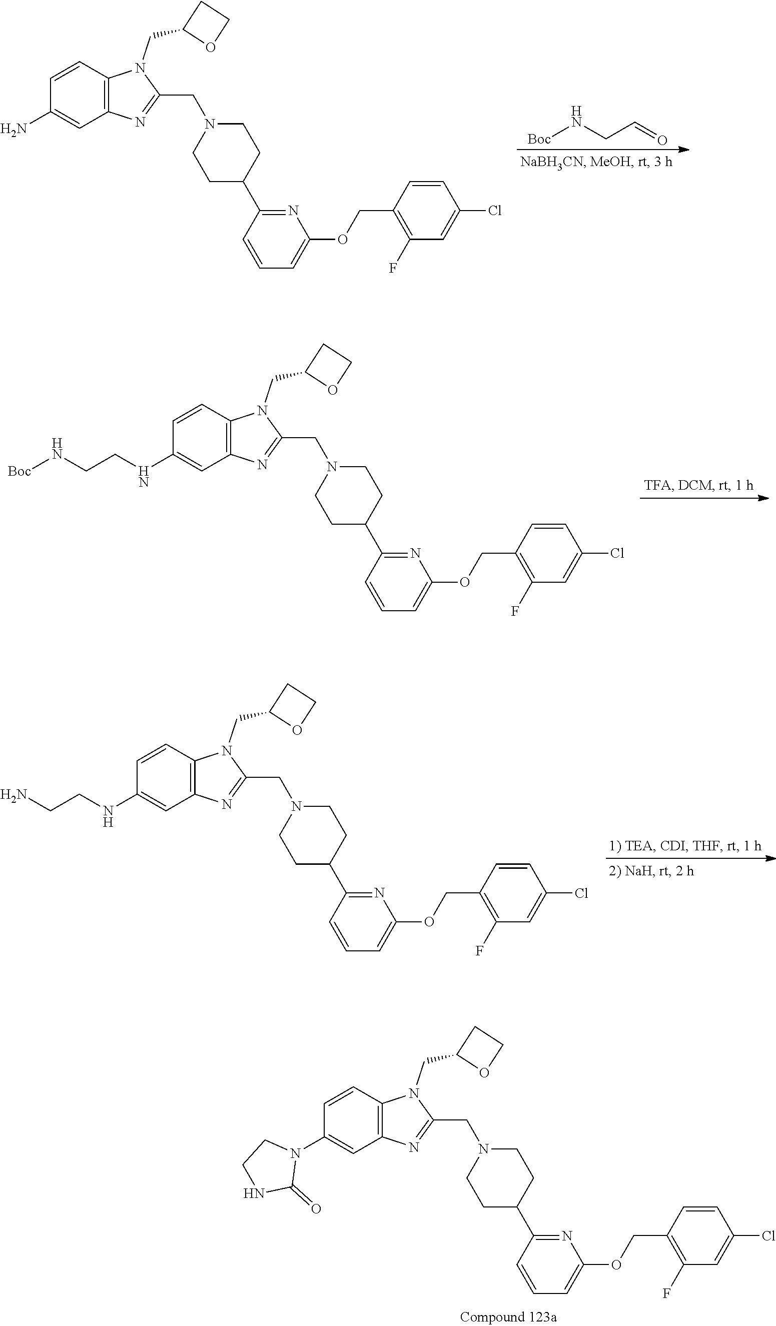 [SMILES: Nc1ccc2c(c1)nc(CN1CCC(c3cccc(OCc4ccc(Cl)cc4F)n3)CC1)n2C[C@@H]1CCO1 ; CC(C)(C)OC(=O)NCC=O ; CC(C)(C)OC(=O)NCCNc1ccc2c(c1)nc(CN1CCC(c3cccc(OCc4ccc(Cl)cc4F)n3)CC1)n2C[C@@H]1CCO1 ; NCCNc1ccc2c(c1)nc(CN1CCC(c3cccc(OCc4ccc(Cl)cc4F)n3)CC1)n2C[C@@H]1CCO1 ; O=C1NCCN1c1ccc2c(c1)nc(CN1CCC(c3cccc(OCc4ccc(Cl)cc4F)n3)CC1)n2C[C@@H]1CCO1]

### Step A: tert-butyl (S)-(2-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)amino)ethyl)carbamate

To a solution of (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (100 mg, 0.190 mmol) in MeOH (3 mL) was added tert-butyl (2-oxoethyl)carbamate (60 mg, 0.370 mmol) and NaBH₃CN (72 mg, 1.140 mmol). The solution was stirred at room temperature for 3 hours. The mixture was quenched with H₂O, extracted with DCM. The combined organic layer was dried over sodium sulfate, filtered and concentrated under reduced pressure. The residue was purified by column chromatography (DCM/MeOH=20/1) to give tert-butyl (S)-(2-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)amino)ethyl)carbamate (70 mg, yield: 55%) as a colorless oil.

MS Calcd.: 678.3; MS Found: 679.0 [M+H]⁺.

### Step B: (S)—N1-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)ethane-1,2-diamine

To a solution of tert-butyl (S)-(2-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)amino)ethyl)carbamate (70 mg, 0.103 mmol) in DCM (2 mL) was added TFA (0.2 mL). The solution was stirred at room temperature for 60 minutes. The reaction mixture was concentrated under vacuum to give (S)—N1-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)ethane-1,2-diamine (73 mg) as colorless oil. The crude product was used for the next step without further purification.

MS Calcd.: 578.3; MS Found: 579.1 [M+H]⁺.

### Step C: 1-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}imidazolidin-2-one (Compound 123a)

To a solution of (S)—N1-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)ethane-1,2-diamine (73 mg) in THE (2 mL) was added TEA (4.2 mg, 0.042 mmol) and CDI (3 mg, 0.020 mmol). The resulting mixture was stirred at room temperature for 1 hour, then cooled to 0° C. NaH (1.7 mg, 0.042 mmol, 60% purity) was added. The resulting mixture was stirred at room temperature for 2 hours, quenched with water. The mixture was purified by Prep-HPLC to give Compound 123a (27.63 mg, yield: 40%) as a white solid.

MS Calcd.: 604.2; MS Found: 605.0 [M+H]⁺.

¹H NMR (400 MHz, DMSO-d₆): δ 7.76 (br s, 1H), 7.69 (t, J=8.0 Hz, 1H), 7.63-7.67 (m, 2H), 7.60 (t, J=8.4 Hz, 1H), 7.48 (dd, J=10.0 Hz, J=1.6, 1H), 7.32 (dd, J=8.0 Hz, J=1.6 Hz, 1H), 6.93 (d, J=7.2 Hz, 1H), 6.88 (s, 1H), 6.74 (d, J=8.0 Hz, 1H), 5.40 (s, 2H), 5.01-5.08 (m, 1H), 4.65-4.73 (m, 1H), 4.52-4.59 (m, 1H), 4.46-4.51 (m, 1H), 4.27-4.34 (m, 1H), 3.88-3.94 (m, 2H), 3.55-3.77 (m, 2H), 3.42 (t, J=8.4 Hz, 4H), 2.86-3.19 (m, 3H), 2.62-2.73 (m, 1H), 2.28-2.36 (m, 1H), 1.98-2.11 (m, 4H).

### Example 78: 2-[(4-{6-[(4-chloro-2-fluorophenyl)methoxy]pyridin-2-yl}piperidin-1-yl)methyl]-5-(1-methyl-1H-pyrazol-3-yl)-1-{[(2S)-oxetan-2-yl]methyl}-1H-1,3-benzodiazole (Compound 124a)

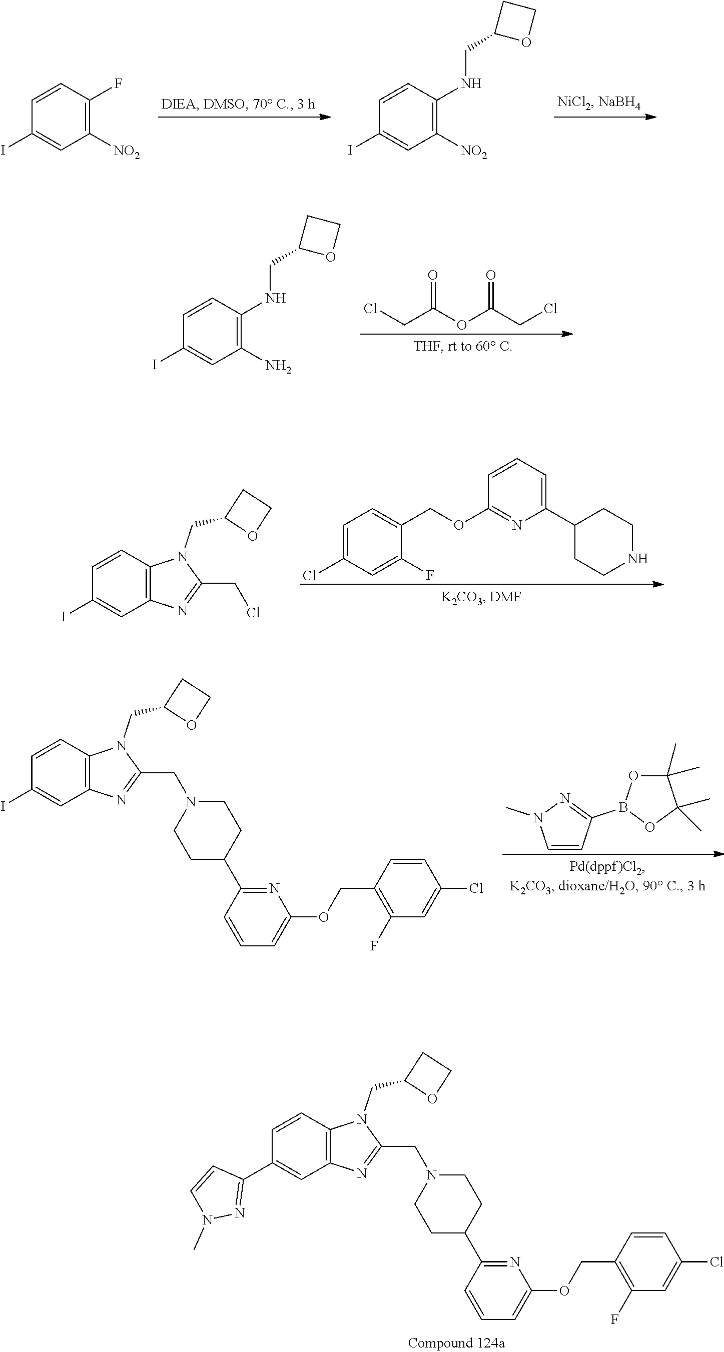

### Step A: (S)-4-iodo-2-nitro-N-(oxetan-2-ylmethyl)aniline

To a solution of 1-fluoro-4-iodo-2-nitrobenzene (1 g, 3.75 mmol) and (S)-oxetan-2-ylmethanamine MsOH salt (820 mg, 4.48 mmol) in DMSO (10 mL) was added DIEA (1.44 g, 11.2 mmol) at room temperature. The reaction was stirred at 70° C. for 3 hours. After the reaction was completed, the reaction was quenched with water (50 mL) and extracted with ethyl acetate (100 mL×3). The organic layer was combined and washed with brine (100 mL×2), dried over sodium sulfate, filtered and concentrated in vacuum. The residue was purified by column chromatography to give (S)-4-iodo-2-nitro-N-(oxetan-2-ylmethyl)aniline (1.3 g, 96% yield) as a yellow solid.

MS Calcd.: 334.0; MS Found: 334.9 [M+H]⁺.

### Step B: (S)-4-iodo-N1-(oxetan-2-ylmethyl)benzene-1,2-diamine

To a solution of (S)-4-iodo-2-nitro-N-(oxetan-2-ylmethyl)aniline (700 mg, 2.10 mmol) in MeOH (10 mL) was added NiCl₂ (543 mg, 4.20 mmol) at 0° C. Then NaBH₄ (238 mg, 6.3 mmol) was added slowly. After the reaction was completed, the reaction was quenched with water (50 mL) and extracted with ethyl acetate (100 mL×3). The organic layer was combined and washed with brine (100 mL×2), dried over sodium sulfate, filtered and concentrated in vacuum. The residue was purified by column chromatography to give (S)-4-iodo-N1-(oxetan-2-ylmethyl)benzene-1,2-diamine (540 mg, 85% yield) as a yellow solid.

MS Calcd.: 304.0; MS Found: 305.0 [M+H]⁺.

### Step C: (S)-2-(chloromethyl)-5-iodo-1-(oxetan-2-ylmethyl)-1H-benzo[d]imidazole

To a solution of (S)-4-iodo-N1-(oxetan-2-ylmethyl)benzene-1,2-diamine (440 mg, 1.45 mmol) in THE (5 mL) was added 2-chloroacetic anhydride (270 mg, 1.59 mmol). The reaction was stirred at room temperature for 1 hour. The reaction was monitored by LCMS which showed the starting material was consumed. Then the reaction was stirred at 60° C. for 16 hours. After the reaction was completed, the reaction was filtered and concentrated to give crude product which was purified by column chromatography to give (S)-2-(chloromethyl)-5-iodo-1-(oxetan-2-ylmethyl)-1H-benzo[d]imidazole (530 mg, crude, 80% purity) as yellow oil.

MS Calcd.: 362.0; MS Found: 362.8 [M+H]⁺.

### Step D: (S)-2-((4-(6-((4-chloro-2-fluorobenzyl)oxy)pyridin-2-yl)piperidin-1-yl)methyl)-5-iodo-1-(oxetan-2-ylmethyl)-1H-benzo[d]imidazole

To a solution of (S)-2-(chloromethyl)-5-iodo-1-(oxetan-2-ylmethyl)-1H-benzo[d]imidazole (500 mg, 1.38 mmol) and 2-((4-chloro-2-fluorobenzyl)oxy)-6-(piperidin-4-yl)pyridine (542 mg) in DMF (5 mL) was added K₂CO₃ (572 mg, 4.14 mmol). The reaction was stirred at 50° C. for 2 hours. After the reaction was completed, the reaction was quenched with water (20 mL) and extracted with ethyl acetate (30 mL×3). The organic layer was combined and washed with brine (50 mL×2), dried over sodium sulfate, filtered and concentrated in vacuum to give crude product which was purified by column chromatography to give (S)-2-((4-(6-((4-chloro-2-fluorobenzyl)oxy)pyridin-2-yl)piperidin-1-yl)methyl)-5-iodo-1-(oxetan-2-ylmethyl)-1H-benzo[d]imidazole (400 mg, 56% yield) as yellow oil.

MS Calcd.: 646.1; MS Found: 646.9 [M+H]⁺.

### Step E: 2-[(4-{6-[(4-chloro-2-fluorophenyl)methoxy]pyridin-2-yl}piperidin-1-yl)methyl]-5-(1-methyl-1H-pyrazol-3-yl)-1-{[(2S)-oxetan-2-yl]methyl}-1H-1,3-benzodiazole (Compound 124a)

A mixture of (S)-2-((4-(6-((4-chloro-2-fluorobenzyl)oxy)pyridin-2-yl)piperidin-1-yl)methyl)-5-iodo-1-(oxetan-2-ylmethyl)-1H-benzo[d]imidazole (50 mg, 0.08 mmol), 1-methyl-3-(4,4,5,5-tetramethyl-1,3,2-dioxaborolan-2-yl)-1H-pyrazole (17.7 mg, 0.09 mmol), Pd(dppf)Cl₂ (6 mg, 0.01 mmol) and K₂CO₃ (32 mg, 0.24 mmol), in dioxane (2 mL)/H₂O (0.5 mL) was degassed and charged with N₂. The reaction was stirred at 90° C. for 3 hours. After the reaction was completed, the reaction was filtered and directly purified by Prep-HPLC to give Compound 124a (4.72 mg, 10% yield) as a white solid.

MS Calcd.: 600.2; MS Found: 601.4 [M+H]⁺.

¹H NMR (400 MHz, DMSO-d₆): δ 7.94 (d, J=0.8 Hz, 1H), 7.67-7.72 (m, 2H), 7.60-7.65 (m, 2H), 7.56 (t, J=8.0 Hz, 1H), 7.46 (dd, J=10.0 Hz, 2.0 Hz, 1H), 7.30 (dd, J=8.4 Hz, 2.0 Hz, 1H), 6.88 (d, J=6.8 Hz, 1H), 6.65-6.68 (m, 2H), 5.38 (s, 2H), 5.11-5.14 (m, 1H), 4.65-4.75 (m, 1H), 4.57-4.62 (m, 1H), 4.45-4.49 (m, 1H), 4.37-4.41 (m, 1H), 3.88-3.93 (m, 4H), 3.76 (d, J=13.6 Hz, 1H), 2.98-3.02 (m, 1H), 2.85-2.89 (m, 1H), 2.56-2.74 (m, 2H), 2.40-2.46 (m, 1H), 2.14-2.26 (m, 2H), 1.67-1.79 (m, 4H).

### Example 79: 2-[(4-{6-[(4-chloro-2-fluorophenyl)methoxy]pyridin-2-yl}piperidin-1-yl)methyl]-1-{[(2S)-oxetan-2-yl]methyl}-5-(1H-1,2,3,4-tetrazol-5-yl)-1H-1,3-benzodiazole (Compound 125a)

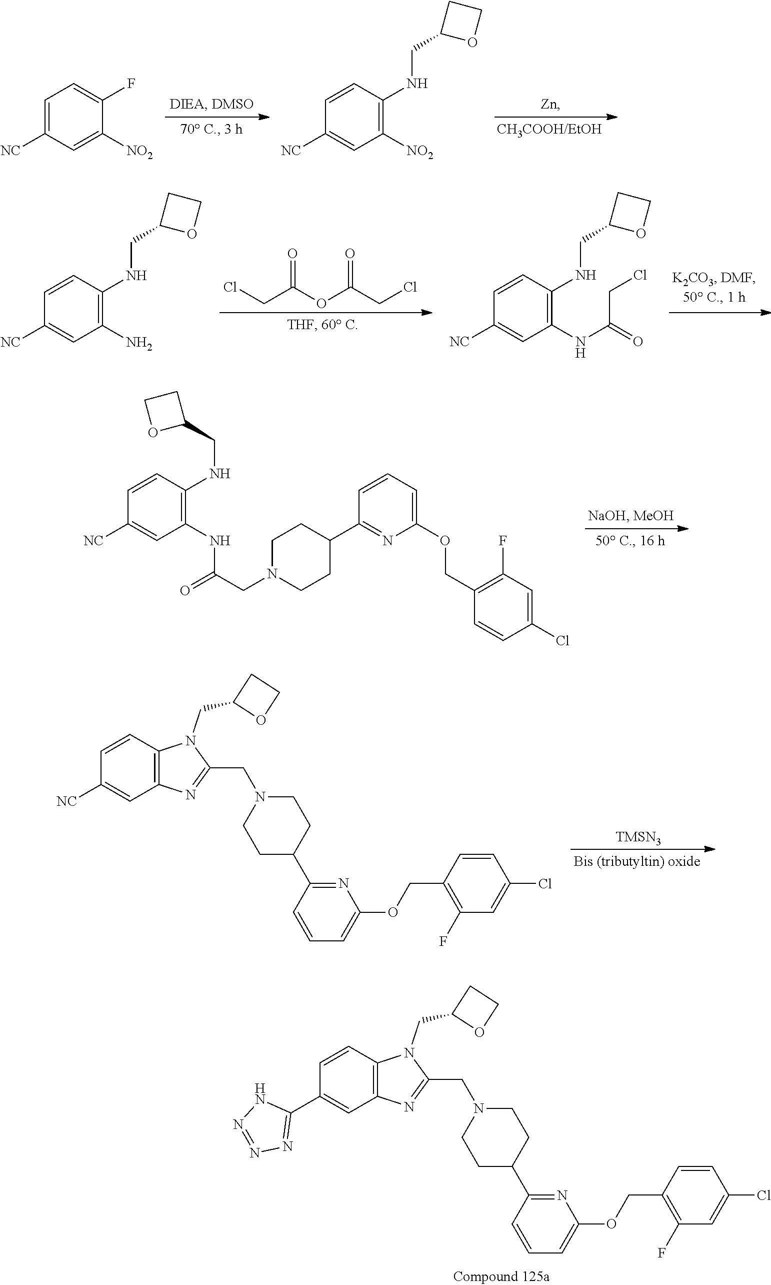

### Step A: (S)-3-nitro-4-((oxetan-2-ylmethyl)amino)benzonitrile

To a solution of 4-fluoro-3-nitrobenzonitrile (300 mg, 1.81 mmol) and (S)-oxetan-2-ylmethanamine MsOH salt (397 mg, 2.17 mmol) in DMSO (3 mL) was added DIEA (699 mg, 5.41 mmol) at room temperature. The reaction was stirred at 70° C. for 3 hours. After the reaction was completed, the reaction was quenched with water (50 mL) and extracted with ethyl acetate (100 mL×3). The organic layer was combined and washed with brine (100 mL×2), dried over sodium sulfate, filtered and concentrated in vacuum. The residue was purified by column chromatography to give (S)-3-nitro-4-((oxetan-2-ylmethyl)amino)benzonitrile (470 mg, crude) as a yellow solid.

MS Calcd.: 233.1; MS Found: 232.0 [M−H]⁻.

### Step B: (S)-3-amino-4-((oxetan-2-ylmethyl)amino)benzonitrile

To a solution of (S)-3-nitro-4-((oxetan-2-ylmethyl)amino)benzonitrile (420 mg, 1.80 mmol) in EtOH (3 mL) and CH₃COOH (1 mL) was added Zn (585 mg, 9.0 mmol). The reaction was stirred at 80° C. for 3 hours. After the reaction was completed, the reaction was filtered and concentrated in vacuum. The residue was purified by column chromatography to give (S)-3-amino-4-((oxetan-2-ylmethyl)amino)benzonitrile (340 mg, 93% yield) as a yellow solid.

MS Calcd.: 203.1; MS Found: 204.0 [M+H]⁺.

### Step C: (S)-2-chloro-N-(5-cyano-2-((oxetan-2-ylmethyl)amino)phenyl)acetamide

To a solution of (S)-3-amino-4-((oxetan-2-ylmethyl)amino)benzonitrile (340 mg, 1.70 mmol) in THE (4 mL) was added 2-chloroacetic anhydride (341 mg, 2.0 mmol). The reaction was stirred at room temperature for 1 hour. Then the reaction was stirred at 60° C. for 16 hours. After the reaction was completed, the reaction was filtered and concentrated to give crude product. The crude product was purified by column chromatography to give (S)-2-chloro-N-(5-cyano-2-((oxetan-2-ylmethyl)amino)phenyl)acetamide (370 mg, crude, 80% purity) as yellow oil.

MS Calcd.: 279.1; MS Found: 280.0 [M+H]⁺.

### Step D: (S)-2-((4-(6-((4-chloro-2-fluorobenzyl)oxy)pyridin-2-yl)piperidin-1-yl)methyl)-5-iodo-1-(oxetan-2-ylmethyl)-1H-benzo[d]imidazole

To a solution of (S)-2-chloro-N-(5-cyano-2-((oxetan-2-ylmethyl)amino)phenyl)acetamide (370 mg, 1.30 mmol) and 2-((4-chloro-2-fluorobenzyl)oxy)-6-(piperidin-4-yl)pyridine (1.17 g) in DMF (4 mL) was added K₂CO₃ (550 mg, 3.90 mmol). The reaction was stirred at 50° C. for 1 hours. After the reaction was completed, the reaction was quenched with water (20 mL) and extracted with ethyl acetate (30 mL×3). The organic layer was combined and washed with brine (50 mL×2), dried over sodium sulfate, filtered and concentrated in vacuum to give crude product. The crude product was purified by column chromatography to give (S)-2-(4-(6-((4-chloro-2-fluorobenzyl)oxy)pyridin-2-yl)piperidin-1-yl)-N-(5-cyano-2-((oxetan-2-ylmethyl)amino)phenyl)acetamide (570 mg, 76% yield) as yellow oil.

MS Calcd.: 563.2; MS Found: 564.0[M+H]⁺.

### Step E: (S)-2-((4-(6-((4-chloro-2-fluorobenzyl)oxy)pyridin-2-yl)piperidin-1-yl)methyl)-1-(oxetan-2-ylmethyl)-1H-benzo[d]imidazole-5-carbonitrile

A solution of (S)-2-(4-(6-((4-chloro-2-fluorobenzyl)oxy)pyridin-2-yl)piperidin-1-yl)-N-(5-cyano-2-((oxetan-2-ylmethyl)amino)phenyl)acetamide (570 mg, 1.00 mmol) in MeOH (4 mL) was added NaOH solution (6 mL, 2 M in water). The reaction was stirred at 50° C. for 16 hours. After the reaction was completed, the reaction was quenched with water (20 mL) and extracted with ethyl acetate (30 mL×3). The organic layer was combined and washed with brine (50 mL×2), dried over sodium sulfate, filtered and concentrated in vacuum to give crude product. The crude product was purified by column chromatography to give (S)-2-((4-(6-((4-chloro-2-fluorobenzyl)oxy)pyridin-2-yl)piperidin-1-yl)methyl)-1-(oxetan-2-ylmethyl)-1H-benzo [d]imidazole-5-carbonitrile (400 mg, 72% yield) as yellow solid.

MS Calcd.: 545.2; MS Found: 546.0[M+H]⁺.

### Step F: 2-[(4-{6-[(4-chloro-2-fluorophenyl)methoxy]pyridin-2-yl}piperidin-1-yl)methyl]-1-{[(2S)-oxetan-2-yl]methyl}-5-(1H-1,2,3,4-tetrazol-5-yl)-1H-1,3-benzodiazole (Compound 125a)

A mixture of (S)-2-((4-(6-((4-chloro-2-fluorobenzyl)oxy)pyridin-2-yl)piperidin-1-yl)methyl)-1-(oxetan-2-ylmethyl)-1H-benzo[d]imidazole-5-carbonitrile (250 mg, 0.46 mmol), TMSN₃ (792 mg, 6.88 mmol), Bis(tributyltin) oxide (228 mg, 0.92 mmol) in toluene (3 mL) was degassed and charged with N₂. The reaction was stirred at 100° C. for 16 hours. After the reaction was completed, the reaction was filtered and concentrated. The residue was purified by Prep-HPLC to give Compound 125a (1.12 mg) as a white solid.

MS Calcd.: 588.2; MS Found: 589.4 [M+H]⁺.

¹H NMR (400 MHz, METHANOL-D4): δ 8.36 (s, 1H), 8.04 (dd, J=8.4 Hz, J=1.2 Hz, 1H), 7.74 (d, J=8.8 Hz, 1H), 7.60 (t, J=7.6 Hz, 1H), 7.49 (t, J=8.4 Hz, 1H), 7.18-7.23 (m, 2H), 6.86 (d, J=7.2 Hz, 1H), 6.66 (d, J=8.4 Hz, 1H), 5.43 (s, 2H), 5.24-5.29 (m, 1H), 4.77-4.80 (m, 3H), 4.63-4.72 (m, 3H), 4.43-4.48 (m, 1H), 4.20-4.35 (m, 2H), 3.37-3.40 (m, 2H), 2.65-2.84 (m, 4H), 2.49-2.58 (m, 1H), 2.19 (t, J=7.6 Hz, 1H).

### Example 80: 3-({2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}amino)-4-(methylamino)cyclobut-3-ene-1,2-dione (Compound 208a)

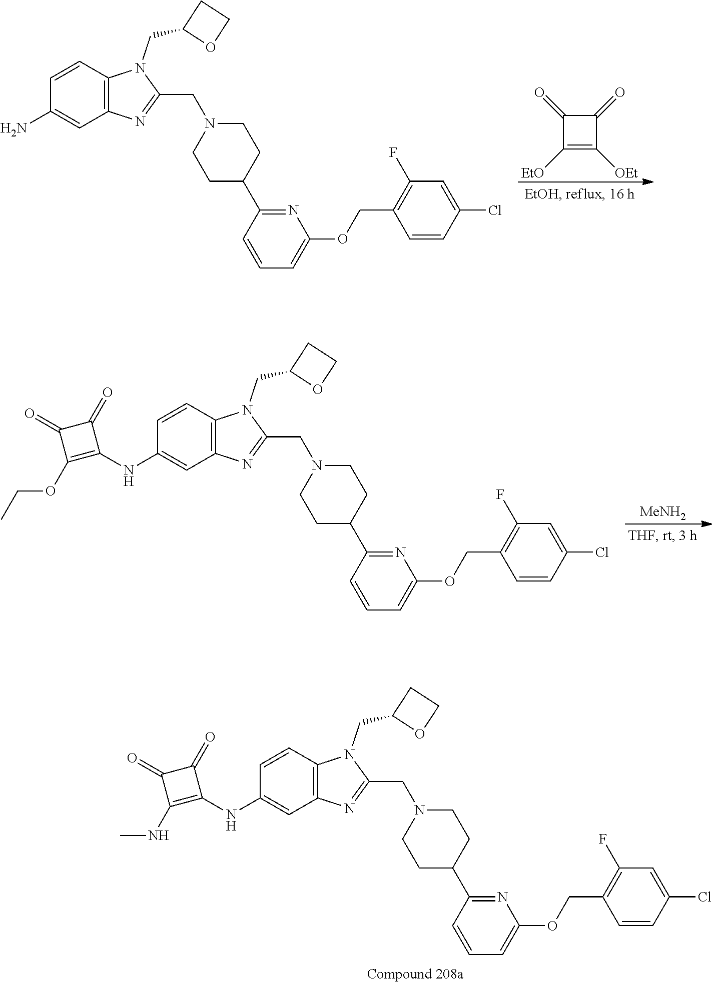

### Step A: (S)-3-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)amino)-4-ethoxycyclobut-3-ene-1,2-dione

A mixture of (S)-2-((4-(6-((4-chloro-2-fluorobenzyl)oxy)pyridin-2-yl)piperidin-1-yl)methyl)-1-(oxetan-2-ylmethyl)-1H-benzo[d]imidazol-5-amine (88 mg, 0.135 mmol), and 3,4-diethoxycyclobut-3-ene-1,2-dione (28 mg, 0.163 mmol) in ethanol (2 ml) was stirred at 80° C. for 16 hours. The mixture was filtered and the filtrate was purified by prep-HPLC to furnish (S)-3-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)amino)-4-ethoxycyclobut-3-ene-1,2-dione (45 mg, yield: 50.4%) as yellow solid.

MS Calcd.: 659.2; MS Found: 660.2 [M+H]⁺.

### Step B: 3-({2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}amino)-4-(methylamino)cyclobut-3-ene-1,2-dione (Compound 208a)

A mixture of (S)-3-((2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)amino)-4-ethoxycyclobut-3-ene-1,2-dione (45 mg, 0.068 mmol) in methylamine tetrahydrofuran solution (6M, 2 mL) was stirred at room temperature for 3 hours. The reaction mixture was purified by prep-HPLC directly to give Compound 208a (4.9 mg, yield: 11.0%) as white solid.

MS Calcd.: 644.2; MS Found: 645.2 [M+H]⁺.

¹H NMR (400 MHz, METHANOL-D4) 67.94 (s, 1H), 7.68-7.62 (m, 2H), 7.51 (t, J=8.4 Hz, 1H), 7.43 (d, J=8.0 Hz, 1H), 7.28-7.20 (m, 2H), 6.92 (d, J=7.2 Hz, 1H), 6.73 (d, J=8.0 Hz, 1H), 5.45 (s, 2H), 5.24 (q, J=7.6 Hz, 1H), 4.81-4.68 (m, 4H), 4.65-4.58 (m, 1H), 4.50-4.45 (m, 1H), 3.90-3.75 (m, 1H), 3.36 (s, 3H), 3.35-3.30 (m, 3H), 3.06-3.01 (m, 1H), 2.84-2.79 (m, 1H), 2.58-2.52 (m, 1H), 2.21-2.11 (m, 4H).

### Example 81: 2-[(4-{6-[(4-chloro-2-fluorophenyl)methoxy]pyridin-2-yl}piperidin-1-yl)methyl]-5-[1-(oxan-3-yl)-1H-1,2,3-triazol-4-yl]-1-{[(2S)-oxetan-2-yl]methyl}-1H-1,3-benzodiazole (Compound 209a)

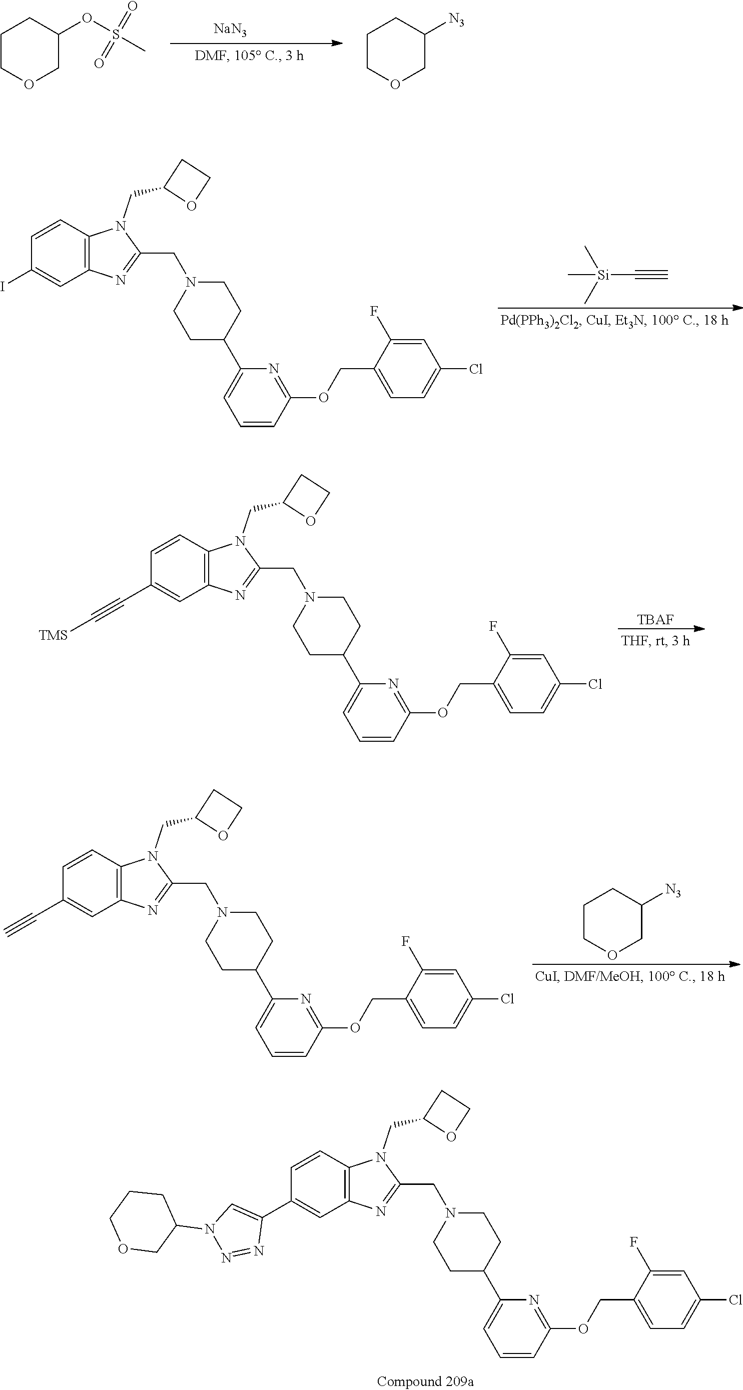

### Step A: 3-azidotetrahydro-2H-pyran

A mixture of tetrahydro-2H-pyran-3-yl methanesulfonate (240 mg, 1.333 mmol), NaN₃ (92 mg, 1.733 mmol) in DMF (2.0 mL) was stirred at 105° C. for 3 h in a sealed tube. The mixture was quenched with brine (20 mL) and extracted with EA (50 mL×3). The combined organic layers were washed with brine (10 mL×1), dried over anhydrous Na₂SO₄ and concentrated under vacuum to furnish 3-azidotetrahydro-2H-pyran (200 mg, crude) as crude yellow oil.

¹H NMR (400 MHz, CDCl₃) δ 3.87-3.83 (m, 1H), 3.77-3.72 (m, 1H), 3.54-3.47 (m, 2H), 3.41-3.37 (m, 1H), 2.09-2.05 (m, 1H), 1.85-1.78 (m, 1H), 1.65-1.58 (m, 2H).

### Step B: (S)-2-((4-(6-((4-chloro-2-fluorobenzyl)oxy)pyridin-2-yl)piperidin-1-yl)methyl)-1-(oxetan-2-ylmethyl)-5-((trimethylsilyl)ethynyl)-1H-benzo[d]imidazole

A stirred mixture of ethynyltrimethylsilane (334 mg, 3.399 mmol), (S)-2-((4-(6-((4-chloro-2-fluorobenzyl)oxy)pyridin-2-yl)piperidin-1-yl)methyl)-5-iodo-1-(oxetan-2-ylmethyl)-1H-benzo[d]imidazole (110 mg, 0.169 mmol), Pd(PPh₃)₂Cl₂ (12 mg, 0.017 mmol) and CuI (3.3 mg, 0.017 mmol) in Et₃N/DMF (1/8, 4.5 mL) was stirred at 100° C. for 18 h under Ar atmosphere. Upon cooling down, the mixture was diluted with brine (50 mL) and extracted with EA (15 mL×3). The combined organic layers were washed with brine (10 mL×1), dried over anhydrous Na₂SO₄ and then concentrated under vacuum. The residue was purified by Prep-TLC to furnish (S)-2-((4-(6-((4-chloro-2-fluorobenzyl)oxy)pyridin-2-yl)piperidin-1-yl)methyl)-1-(oxetan-2-ylmethyl)-5-((trimethylsilyl)ethynyl)-1H-benzo[d]imidazole (185 mg, crude) as a crude yellow oil.

MS Calcd.: 616.2; MS Found: 617.7 [M+H]⁺.

### Step C: (S)-2-((4-(6-((4-chloro-2-fluorobenzyl)oxy)pyridin-2-yl)piperidin-1-yl)methyl)-5-ethynyl-1-(oxetan-2-ylmethyl)-1H-benzo[d]imidazole

A mixture of TBAF (0.9 mL, 0.900 mmol, 1 N in THF) and (S)-2-((4-(6-((4-chloro-2-fluorobenzyl)oxy)pyridin-2-yl)piperidin-1-yl)methyl)-1-(oxetan-2-ylmethyl)-5-((trimethylsilyl)ethynyl)-1H-benzo[d]imidazole (185 mg, 0.300 mmol, crude) in THE (3.0 mL) was stirred at room temperature for 3 h. The reaction mixture was concentrated under vacuum. The residue was purified by Prep-TLC to furnish (S)-2-((4-(6-((4-chloro-2-fluorobenzyl)oxy)pyridin-2-yl)piperidin-1-yl)methyl)-5-ethynyl-1-(oxetan-2-ylmethyl)-1H-benzo[d]imidazole (55 mg, 33.6%) as a crude white solid.

MS Calcd.: 544.2; MS Found: 545.7 [M+H]⁺.

### Step D: 2-[(4-{6-[(4-chloro-2-fluorophenyl)methoxy]pyridin-2-yl}piperidin-1-yl)methyl]-5-[1-(oxan-3-yl)-1H-1,2,3-triazol-4-yl]-1-{[(2S)-oxetan-2-yl]methyl}-1H-1,3-benzodiazole (Compound 209a)

A mixture of (S)-2-((4-(6-((4-chloro-2-fluorobenzyl)oxy)pyridin-2-yl)piperidin-1-yl)methyl)-5-ethynyl-1-(oxetan-2-ylmethyl)-1H-benzo[d]imidazole (55 mg, 0.101 mmol), 3-azidotetrahydro-2H-pyran (200 mg, crude), CuI (3.8 mg, 0.0202 mmol) in DMF/MeOH (5/1, 3.0 ml) was stirred at 100° C. for 18 h under Ar atmosphere. Upon cooling down, the solids were filtrated out. The filtrate was directly purified by Prep-HPLC to furnish Compound 209a (3.9 mg, 5.74%) as a white solid.

MS Calcd.: 671.3; MS Found: 672.8 [M+H]⁺.

¹H NMR (400 MHz, methanol-d₄) δ 8.54 (s, 1H), 8.25 (s, 1H), 7.91 (dd, J=8.2, 1.2 Hz, 1H), 7.75 (d, J=7.2 Hz, 1H), 7.67 (t, J=8.0 Hz, 1H), 7.51 (t, J=8.0 Hz, 1H), 7.26-7.20 (m, 2H), 6.93 (d, J=7.6 Hz, 1H), 6.75 (d, J=8.0 Hz, 1H), 5.44 (s, 2H), 5.26-5.21 (m, 1H), 4.80-4.69 (m, 4H), 4.52-4.47 (m, 1H), 4.18-4.12 (m, 1H), 3.94-3.84 (m, 4H), 3.70-3.60 (m, 2H), 3.48-3.35 (m, 3H), 3.13-3.04 (m, 1H), 2.86-2.75 (m, 1H), 2.58-2.51 (m, 1H), 2.41-2.33 (m, 1H), 2.32-2.21 (m, 1H), 2.20-2.10 (m, 4H), 1.89-1.78 (m, 2H). ¹⁹F-NMR (400 MHz, methanol-d₄) δ: −117.68.

### Example 82: 2-[(4-{6-[(4-chloro-2-fluorophenyl)methoxy]pyridin-2-yl}piperidin-1-yl)methyl]-5-(4-methyl-1H-1,2,3-triazol-1-yl)-1-{[(2S)-oxetan-2-yl]methyl}-1H-1,3-benzodiazole (Compound 210a)

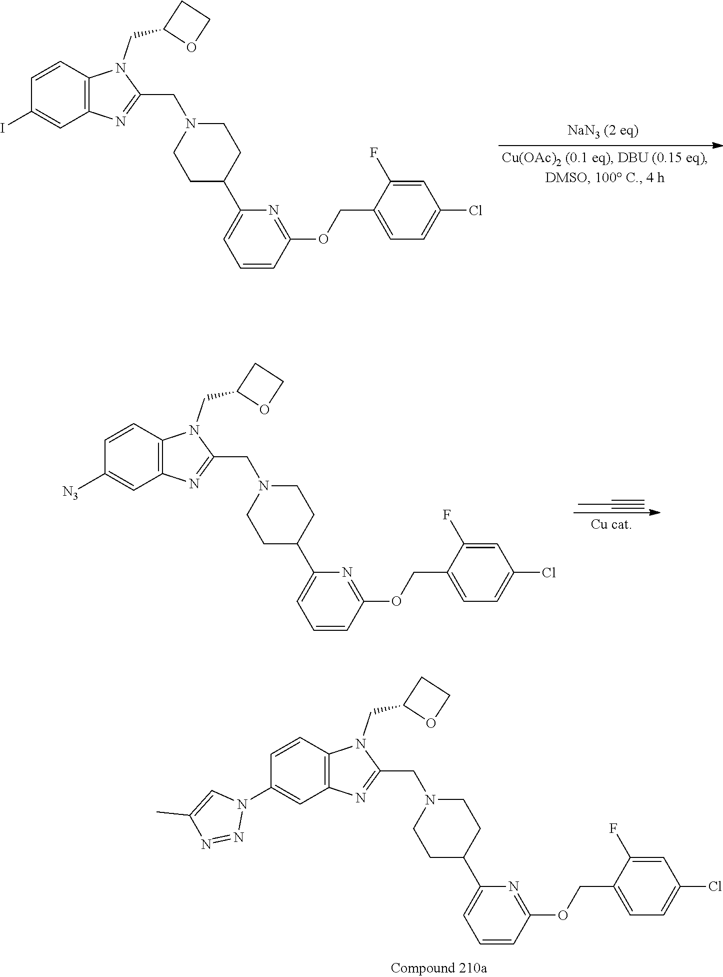

### Step A: (S)-5-azido-2-((4-(6-((4-chloro-2-fluorobenzyl)oxy)pyridin-2-yl)piperidin-1-yl)methyl)-1-(oxetan-2-ylmethyl)-1H-benzo[d]imidazole

A mixture of (S)-2-((4-(6-((4-chloro-2-fluorobenzyl)oxy)pyridin-2-yl)piperidin-1-yl)methyl)-5-iodo-1-(oxetan-2-ylmethyl)-1H-benzo[d]imidazole (100 mg, 0.15 mmol), NaN₃ (32 mg, 0.46 mmol), DBU (1 drop) and Cu(OAc)₂·H₂O (3 mg, 0.015 mmol) in DMSO (2 mL) was stirred at 100° C. under N₂ for 8 hours. Upon cooling down, the mixture was diluted with H2O, extracted with EA, The combined organic layer was washed with brine, dried over Na2SO4, concentracted purified by prep-HPLC (0.1% NH₄HCO₃) to give (S)-5-azido-2-((4-(6-((4-chloro-2-fluorobenzyl)oxy)pyridin-2-yl)piperidin-1-yl)methyl)-1-(oxetan-2-ylmethyl)-1H-benzo[d]imidazole (20 mg, 23% yield) as white solid.

MS Calcd.: 561.2; MS Found: 562.2 [M+H]⁺.

### Step B: 2-[(4-{6-[(4-chloro-2-fluorophenyl)methoxy]pyridin-2-yl}piperidin-1-yl)methyl]-5-(4-methyl-1H-1,2,3-triazol-1-yl)-1-{[(2S)-oxetan-2-yl]methyl}-1H-1,3-benzodiazole (Compound 210a)

A mixture of (S)-5-azido-2-((4-(6-((4-chloro-2-fluorobenzyl)oxy)pyridin-2-yl)piperidin-1-yl)methyl)-1-(oxetan-2-ylmethyl)-1H-benzo[d]imidazole (20 mg, 0.036 mmol), CuI (0.7 mg, 0.0036 mmol) and prop-1-yne (2 mL, 4% in DMF) in MeOH (0.2 mL) was stirred at 100° C. under Ar for 8 hours. The mixture was filtered, the filtrate was purified by prep-HPLC (0.1% NH₄HCO₃) to give Compound 210a (3.4 mg, yield: 15%) as white solid.

MS Calcd.: 601.2; MS Found: 602.2 [M+H]⁺.

¹H NMR (400 MHz, methanol-d₄) δ 8.29 (s, 1H), 8.02 (m, J=1.6 Hz, 1H), 7.85-7.80 (m, 1H), 7.78-7.74 (m, 1H), 7.57 (t, J=7.8 Hz, 1H), 7.50 (t, J=8.0 Hz, 1H), 7.25-7.17 (m, 2H), 6.86 (d, J=8.0 Hz, 1H), 6.64 (d, J=14.0 Hz, 1H), 5.42 (m, 2H), 5.32-5.25 (m, 1H), 4.90-4.85 (m, 1H), 4.75-4.69 (m, 1H), 4.66-4.61 (m, 1H), 4.49-4.43 (m, 1H), 4.05-3.89 (m, 2H), 3.12-3.07 (m, 1H), 2.98-2.95 (m, 1H), 2.86-2.79 (m, 1H), 2.70-2.62 (m, 1H), 2.57-2.50 (m, 1H), 2.42 (s, 3H), 2.39-2.27 (m, 2H), 1.97-1.83 (m, 4H). ¹⁹F-NMR (400 MHz, methanol-d₄) δ −117.64.

### Example 83: 2-[(4-{6-[(4-chloro-2-fluorophenyl)methoxy]pyridin-2-yl}piperidin-1-yl)methyl]-5-[4-(oxan-3-yl)-1H-1,2,3-triazol-1-yl]-1-{[(2S)-oxetan-2-yl]methyl}-1H-1,3-benzodiazole (Compound 211a)

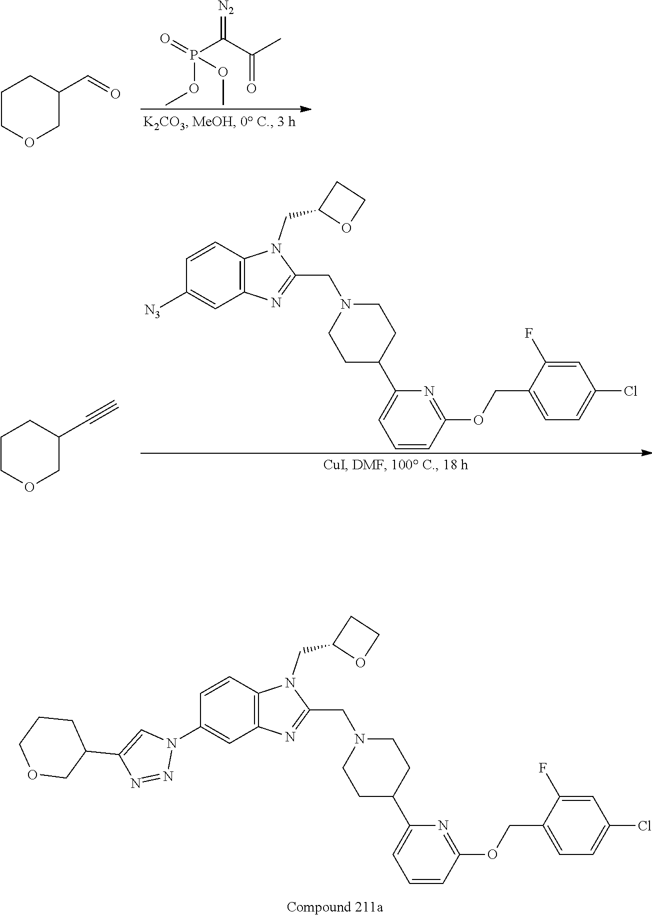

### Step A: 3-ethynyltetrahydro-2H-pyran

To a stirred mixture of oxane-3-carbaldehyde (1.51 g, 13.294 mmol) and dimethyl (1-diazo-2-oxopropyl)phosphonate (3.30 g, 17.198 mmol) in dry MeOH (8.0 mL) was added K₂CO₃ (5.51 g, 39.882 mmol) in portions at 0° C. The reaction mixture was slowly warmed up to room temperature during a period of about 3 h. The mixture was diluted with H₂O and extracted with EtOAc (50 mL×3). The combined organic layers were washed with brine (10 mL×1), dried over anhydrous Na₂SO₄ and then concentrated under vacuum. This furnished 3-ethynyltetrahydro-2H-pyran (580 mg, crude) as crude yellow oil.

¹H NMR (400 MHz, CDCl₃) δ 3.86-3.83 (m, 1H), 3.75-3.30 (m, 2H), 3.43-3.31 (m, 2H), 2.51-2.48 (m, 1H), 2.01 (d, J=1.5 Hz, 1H), 1.64-1.49 (m, 3H).

### Step B: 2-[(4-{6-[(4-chloro-2-fluorophenyl)methoxy]pyridin-2-yl}piperidin-1-yl)methyl]-5-[4-(oxan-3-yl)-1H-1,2,3-triazol-1-yl]-1-{[(2S)-oxetan-2-yl]methyl}-1H-1,3-benzodiazole (Compound 211a)

A mixture of 3-ethynyltetrahydro-2H-pyran (69 mg, crude), (S)-5-azido-2-((4-(6-((4-chloro-2-fluorobenzyl)oxy)pyridin-2-yl)piperidin-1-yl)methyl)-1-(oxetan-2-ylmethyl)-1H-benzo[d]imidazole (20 mg, 0.0356 mmol) and CuI (1.5 mg, 0.0078 mmol) in DMF (1.5 ml) was stirred at 100° C. for 18 h under Ar atmosphere. Upon cooling down, the reaction mixture was filtered and the filtrate was purified by Prep-HPLC to furnish Compound 211a (4.3 mg, yield: 19.1%) as a white solid.

MS Calcd.: 671.3; MS Found: 672.2 [M+H]⁺.

¹H NMR (400 MHz, methanol-d₄) δ 8.43 (s, 1H), 8.05 (s, 1H), 7.87-7.80 (m, 1H), 7.79-7.73 (m, 1H), 7.62-7.43 (m, 2H), 7.24-7.17 (m, 2H), 6.84-6.80 (m, 1H), 6.64-6.60 (m, 1H), 5.43 (s, 2H), 5.32-5.27 (m, 1H), 4.78-4.71 (m, 2H), 4.68-4.58 (m, 2H), 4.53-4.45 (m, 1H), 4.16-4.09 (m, 1H), 4.07-4.00 (m, 1H), 3.96-3.87 (m, 2H), 3.63-3.52 (m, 2H), 3.15-3.04 (m, 3H), 2.98-2.91 (m, 1H), 2.86-2.77 (m, 1H), 2.69-2.61 (m, 1H), 2.59-2.51 (m, 1H), 2.36-2.18 (m, 3H), 1.89-1.83 (m, 3H), 1.79-1.73 (m, 2H). ¹⁹F-NMR (400 MHz, methanol-d₄) −117.677.

### Example 84: 6-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}pyridin-2-amine (Compound 212a)

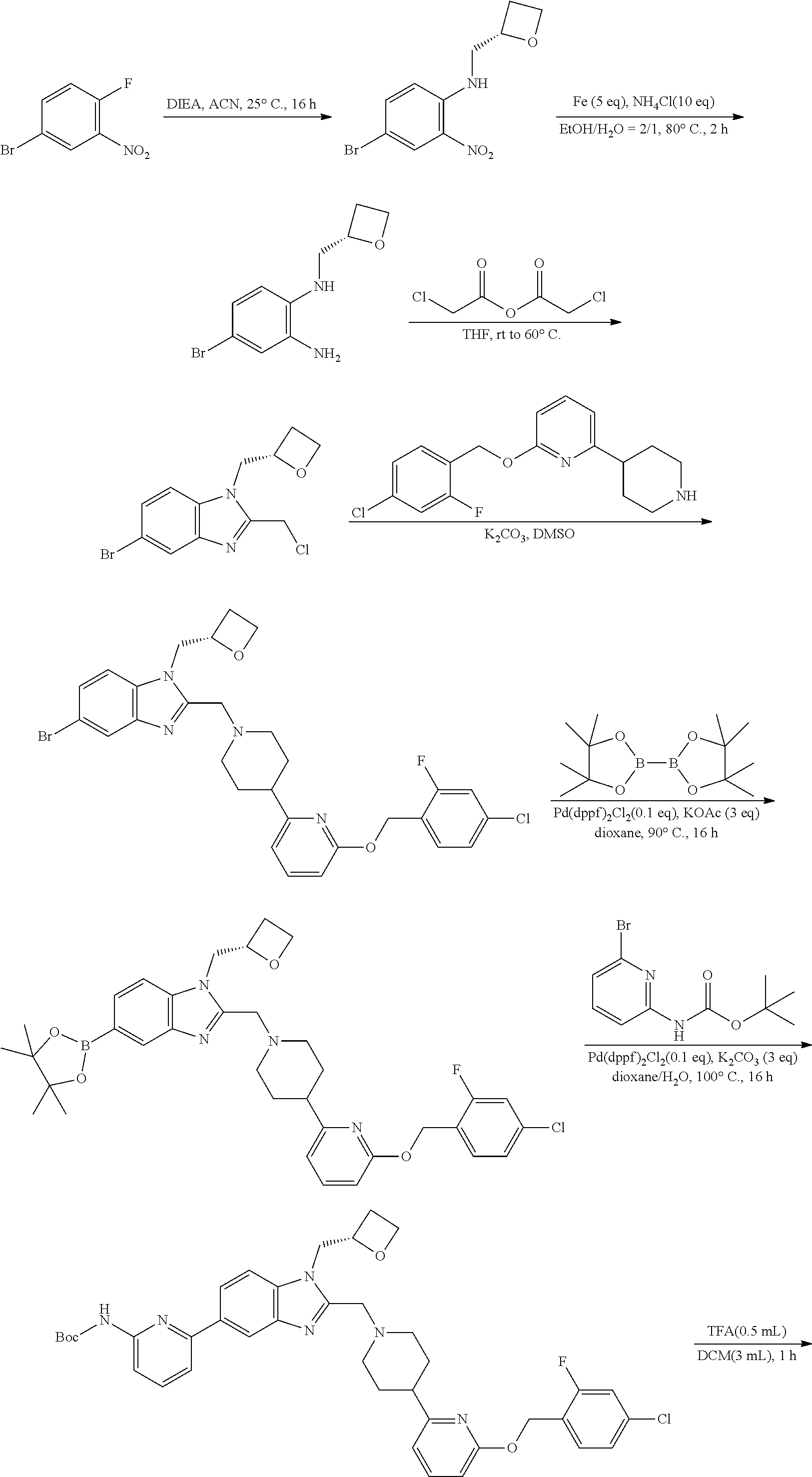

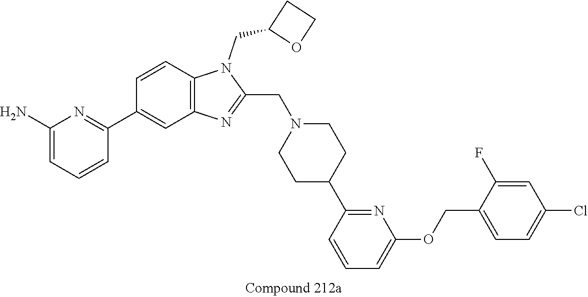 [SMILES: Nc1cccc(-c2ccc3c(c2)nc(CN2CCC(c4cccc(OCc5ccc(Cl)cc5F)n4)CC2)n3C[C@@H]2CCO2)n1]

### Step A: (S)-4-bromo-2-nitro-N-(oxetan-2-ylmethyl)aniline

To a solution of 4-bromo-1-fluoro-2-nitrobenzene (7.0 g, 31.8 mmol) and (S)-oxetan-2-ylmethanamine MsOH salt (5.8 g, 31.8 mmol) in ACN (70 mL) was added DIEA (20.5 g, 159.0 mmol) at room temperature. The reaction was stirred at 25° C. for 16 hours. After the reaction was completed, the reaction was quenched with water (50 mL) and extracted with ethyl acetate (100 mL×3). The organic layer was combined and washed with brine (100 mL×2), dried over sodium sulfate, filtered and concentrated to give (S)-4-bromo-2-nitro-N-(oxetan-2-ylmethyl)aniline (9.08 g, 98.6% yield) as a orange solid.

MS Calcd.: 286.0; MS Found: 287.0 [M+H]⁺.

### Step B: (S)-4-bromo-N1-(oxetan-2-ylmethyl)benzene-1,2-diamine

To a solution of (S)-4-bromo-2-nitro-N-(oxetan-2-ylmethyl)aniline (9.08 g, 31.6 mmol) in EtOH (90 mL) and H₂O (45 mL) was added Fe (8.86 g, 158.2 mmol) and NH₄Cl (16.91 g, 316 mmol). The reaction was stirred at 80° C. for 2 hours. After the reaction was completed, the reaction was filtered and quenched with water (50 mL), extracted with ethyl acetate (100 mL×3). The organic layer was washed with brine (100 mL×2), dried over sodium sulfate, filtered and concentrated in vacuum to give (S)-4-bromo-N1-(oxetan-2-ylmethyl)benzene-1,2-diamine (7.7 mg, 94.8% yield) as a black oil.

MS Calcd.: 256.0; MS Found: 256.9 [M+H]⁺.

### Step C: (S)-5-bromo-2-(chloromethyl)-1-(oxetan-2-ylmethyl)-1H-benzo[d]imidazole

To a solution of (S)-4-bromo-N1-(oxetan-2-ylmethyl)benzene-1,2-diamine (7.7 g, 29.96 mmol) in THE (80 mL) was added 2-chloroacetic anhydride (5.53 g, 32.36 mmol) at room temperature. The reaction was stirred at 60° C. for 16 hours, 2-chloroacetic anhydride was added (5.53 g, 32.36 mmol) and then the reaction was stirred at 60° C. for 16 hours. After the reaction was completed, the reaction was filtered and concentrated to give crude product which was purified by column chromatography to give (S)-5-bromo-2-(chloromethyl)-1-(oxetan-2-ylmethyl)-1H-benzo[d]imidazole (6.6 g, crude) as black oil.

MS Calcd.: 316.0; MS Found: 316.9 [M+H]⁺.

### Step D: (S)-5-bromo-2-((4-(6-((4-chloro-2-fluorobenzyl)oxy)pyridin-2-yl)piperidin-1-yl)methyl)-1-(oxetan-2-ylmethyl)-1H-benzo[d]imidazole

To a solution of 2-((4-chloro-2-fluorobenzyl)oxy)-6-(piperidin-4-yl)pyridine HCl salt (1.5 g, 3.8 mmol) in DMSO (15 mL) was added K₂CO₃ (2.6 g, 19.1 mmol) and Et₃N (1.2 g, 11.4 mmol). The reaction was stirred at room temperature for 10 mins, (S)-5-bromo-2-(chloromethyl)-1-(oxetan-2-ylmethyl)-1H-benzo[d]imidazole (1.2 mg, 3.8 mmol) was added and the reaction was stirred at 60° C. for 3 hours. After the reaction was completed, the reaction was quenched with water (20 mL) and extracted with ethyl acetate (30 mL×3). The organic layer was combined and washed with brine (50 mL×2), dried over sodium sulfate, filtered and concentrated in vacuum to give crude product which was purified by column chromatography to give (S)-5-bromo-2-((4-(6-((4-chloro-2-fluorobenzyl)oxy)pyridin-2-yl)piperidin-1-yl)methyl)-1-(oxetan-2-ylmethyl)-1H-benzo[d]imidazole (1.15 g, crude) as yellow solid.

MS Calcd.: 598.1; MS Found: 599.1 [M+H]⁺.

### Step E: (S)-2-((4-(6-((4-chloro-2-fluorobenzyl)oxy)pyridin-2-yl)piperidin-1-yl)methyl)-1-(oxetan-2-ylmethyl)-5-(4,4,5,5-tetramethyl-1,3,2-dioxaborolan-2-yl)-1H-benzo[d]imidazole

A mixture of (S)-5-bromo-2-((4-(6-((4-chloro-2-fluorobenzyl)oxy)pyridin-2-yl)piperidin-1-yl)methyl)-1-(oxetan-2-ylmethyl)-1H-benzo[d]imidazole (500 mg, 0.84 mmol), 4,4,4′,4′,5,5,5′,5′-octamethyl-2,2′-bi(1,3,2-dioxaborolane) (531 mg, 2.09 mmol), KOAc (247 mg, 2.52 mmol) and Pd(dppf)Cl₂ (61 mg, 0.084 mmol) in dioxane (10 mL) was stirred at 90° C. under N₂ for 16 hours. After the reaction was completed, the reaction was concentrated in vacuum to give crude product which was purified by column chromatography (Al₂O₃ in base, DCM:PE:MeOH=150:150:1-100:100:1) to give (S)-2-((4-(6-((4-chloro-2-fluorobenzyl)oxy)pyridin-2-yl)piperidin-1-yl)methyl)-1-(oxetan-2-ylmethyl)-5-(4,4,5,5-tetramethyl-1,3,2-dioxaborolan-2-yl)-1H-benzo[d]imidazole (190 mg, 35% yield) as black oil.

MS Calcd.: 646.3; MS Found: 647.3 [M+H]⁺.

### Step F: (S)-tert-butyl (6-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)pyridin-2-yl)carbamate

A mixture of (S)-2-((4-(6-((4-chloro-2-fluorobenzyl)oxy)pyridin-2-yl)piperidin-1-yl)methyl)-1-(oxetan-2-ylmethyl)-5-(4,4,5,5-tetramethyl-1,3,2-dioxaborolan-2-yl)-1H-benzo[d]imidazole (80 mg, 0.12 mmol), tert-butyl (6-bromopyridin-2-yl)carbamate (101 mg, 0.37 mmol), K₂CO₃ (51 mg, 0.37 mmol) and Pd(dppf)Cl₂ (9 mg, 0.012 mmol) in dioxane (2.5 mL) and H₂O (0.5 mL) was stirred at 100° C. under N₂ for 16 hours. Upon cooling down, the reaction was concentrated under vacuum, purified by prep-TLC (DCM:MeOH=30/1, twice) to give (S)-tert-butyl (6-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)pyridin-2-yl)carbamate (24 mg, 28% yield) as a yellow oil.

MS Calcd.: 712.3; MS Found: 713.3 [M+H]⁺.

### Step G: 6-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}pyridin-2-amine (Compound 212a)

To a solution of (S)-tert-butyl (6-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)pyridin-2-yl)carbamate (24 mg, 0.034 mmol) in DCM (3 mL) was added YH0.5 mL), the mixture was stirred at 25° C. for 1 hour. The mixture was concentrated under vacuum, diluted with DMF, purified by Preparative-HPLC (0.1% NH₄HCO₃) to give Compound 212a (7.3 mg, yield: 35%) as white solid.

MS Calcd.: 612.2; MS Found: 613.2 [M+H]⁺.

¹H NMR (400 MHz, methanol-d₄) δ 8.02 (d, J=1.2 Hz, 1H), 7.72 (dd, J1=1.2 Hz, J2=8.4 Hz, 1H), 7.58 (d, J=8.8 Hz, 1H), 7.50-7.38 (m, 3H), 7.13-7.07 (m, 2H), 6.96 (d, J=7.2 Hz, 1H), 6.73 (d, J=7.6 Hz, 1H), 6.53 (d, J=8.0 Hz, 1H), 6.42 (d, J=8.4 Hz, 1H), 5.32 (s, 2H), 5.23-5.17 (m, 1H), 4.78-4.70 (m, 1H), 4.65-4.60 (m, 1H), 4.58-4.51 (m, 1H), 4.40-4.34 (m, 1H), 3.92-3.78 (m, 2H), 3.00-2.95 (m, 1H), 2.90-2.84 (m, 1H), 2.75-2.67 (m, 1H), 2.59-2.43 (m, 2H), 2.26-2.13 (m, 2H), 1.82-1.74 (m, 4H).

¹⁹F NMR (400 MHz, methanol-d₄) δ −117.65.

### Example 85: 2-[(4-{6-[(4-chloro-2-fluorophenyl)methoxy]pyridin-2-yl}piperidin-1-yl)methyl]-1-{[(2S)-oxetan-2-yl]methyl}-5-(2H-1,2,3-triazol-4-yl)-1H-1,3-benzodiazole (Compound 149a)

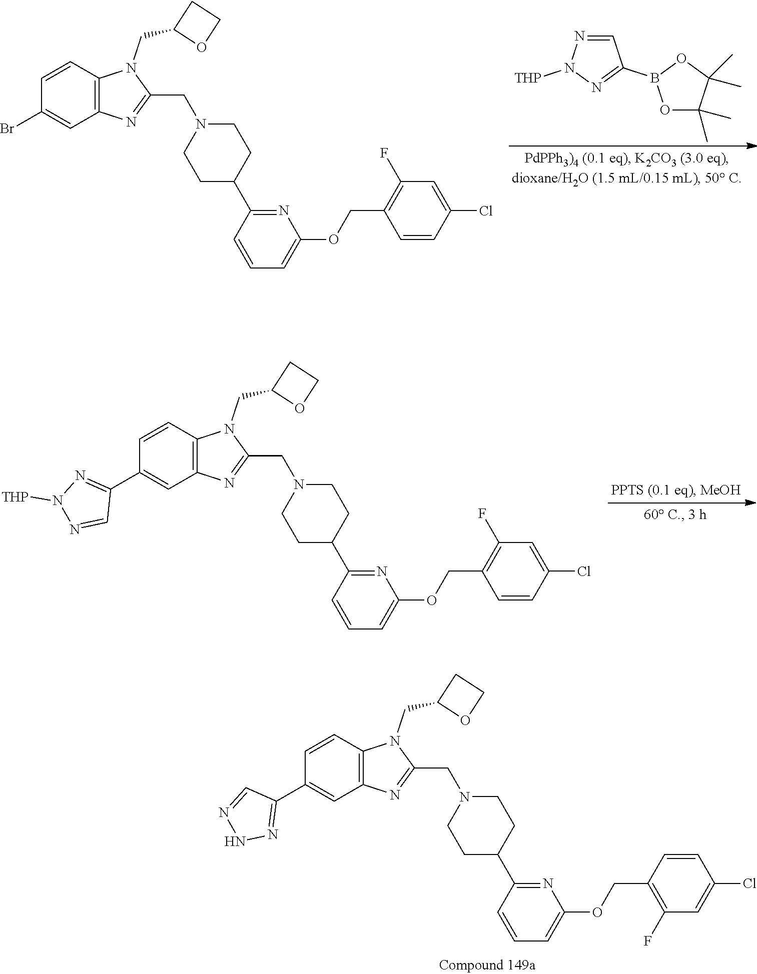

### Step A: 2-((4-(6-((4-chloro-2-fluorobenzyl)oxy)pyridin-2-yl)piperidin-1-yl)methyl)-1-((S)-oxetan-2-ylmethyl)-5-(2-(tetrahydro-2H-pyran-2-yl)-2H-1,2,3-triazol-4-yl)-1H-benzo[d]imidazole

A mixture of (S)-5-bromo-2-((4-(6-((4-chloro-2-fluorobenzyl)oxy)pyridin-2-yl)piperidin-1-yl)methy 1)-1-(oxetan-2-ylmethyl)-1H-benzo[d]imidazole (30 mg, 0.05 mmol) and 2-(tetrahydro-2H-pyran-2-yl)-4-(4,4,5,5-tetramethyl-1,3,2-dioxaborolan-2-yl)-2H-1,2,3-triazole (28 mg, 0.10 mmol), Pd(PPh₃)₄ (6 mg, 0.005 mmol) and K₂CO₃ (21 mg, 0.15 mmol) in dioxane (1.5 ml) and water (0.15 ml) was stirred at 70° C. under N₂ for 18 hours. The reaction mixture was filtered, the filtrate was concentrated in vacuum, purified by prep-TLC (PE/EA=1/2) to give 2-((4-(6-((4-chloro-2-fluorobenzyl)oxy)pyridin-2-yl)piperid in-1-yl)methyl)-1-((S)-oxetan-2-ylmethyl)-5-(2-(tetrahydro-2H-pyran-2-yl)-2H-1,2,3-triazol-4-yl)-1H-benzo[d]imidazole (32 mg, yield: 44.8%) as dark oil.

MS Calcd.: 671.3; MS Found: 672.5 [M+H]⁺.

### Step B: 2-[(4-{6-[(4-chloro-2-fluorophenyl)methoxy]pyridin-2-yl}piperidin-1-yl)methyl]-1-{[(2S)-oxetan-2-yl]methyl}-5-(2H-1,2,3-triazol-4-yl)-1H-1,3-benzodiazole (Compound 149a)

To a solution of 2-((4-(6-((4-chloro-2-fluorobenzyl)oxy)pyridin-2-yl)piperid in-1-yl)methyl)-1-((S)-oxetan-2-ylmethyl)-5-(2-(tetrahydro-2H-pyran-2-yl)-2H-1,2,3-triazol-4-yl)-1H-benzo[d]imidazole (32 mg, 0.048 mmol) in MeOH (5 ml) was added PPTS (1 mg, 0.0005 mmol), the mixture was stirred at 60° C. for 3 hours. The reaction mixture was purified by prep-HPLC directly to give Compound 149a (4.3 mg, yield: 15.4%) as white solid.

MS Calcd.: 587.2; MS Found: 588.2 [M+H]⁺.

¹H NMR (400 MHz, methanol-d₄)) δ 8.16 (s, 1H), 8.11-8.08 (m, 1H), 7.82-7.78 (m, 1H), 7.72-7.68 (m, 1H), 7.59-7.55 (m, 1H), 7.49 (t, J=8.4 Hz, 1H), 7.23-7.15 (m, 2H), 6.85-6.81 (m, 1H), 6.64-6.60 (m, 1H), 5.42-5.38 (m, 2H), 5.31-5.25 (m, 1H), 4.84-4.81 (m, 1H), 4.74-4.69 (m, 1H), 4.64-4.60 (m, 1H), 4.52-4.40 (m, 1H), 3.99 (d, J=13.6 Hz, 1H), 3.91-3.86 (m, 1H), 3.13-3.07 (m, 1H), 2.96-2.90 (m, 1H), 2.83-2.78 (m, 1H), 2.67-2.60 (m, 1H), 2.60-2.50 (m, 1H), 2.34-2.20 (m, 2H), 1.90-1.80 (m, 4H).

### Example 86: 2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazole-5-carboxylic acid (Compound 213a)

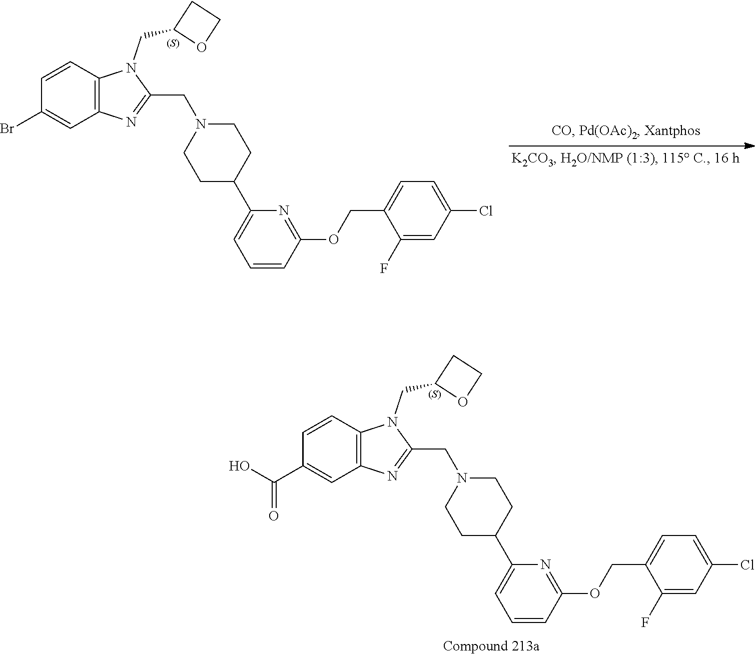

A mixture of (S)-5-bromo-2-((4-(6-((4-chloro-2-fluorobenzyl)oxy)pyridin-2-yl)piperidin-1-yl)methyl)-1-(oxetan-2-ylmethyl)-1H-benzo[d]imidazole (100 mg, 0.167 mmol), Pa(OAc)₂ (7 mg, 0.033 mmol), K₂CO₃ (46 mg, 0.333 mmol) and Xantphos (39 mg, 0.067 mmol) in NMP (1.5 mL) and H₂O (0.5 mL) was stirred at 115° C. for 16 hours under an atmosphere of CO. After the reaction was completed, the mixture was diluted with water, extracted with ethyl acetate. The organic layer was dried over Na₂SO₄ and concentrated under vacuum to give 90 mg crude product. The crude product (45 mg) was purified by Prep-HPLC to give Compound 213a (2.98 mg, yield: 3%) as a white solid.

MS Calcd.: 564.2; MS Found: 565.4 [M+H]⁺.

¹H NMR (400 MHz, DMSO-d₆): 8.17 (d, J=1.2 Hz, 1H), 7.85 (dd, J=8.4 Hz, 1.6 Hz, 1H), 7.69 (d, J=8.4 Hz, 1H), 7.61-7.68 (m, 1H), 7.56 (t, J=8.0 Hz, 1H), 7.46 (dd, J=10.0 Hz, 2.4 Hz, 1H), 7.29 (dd, J=8.4 Hz, 2.0 Hz, 1H), 6.88 (d, J=7.2 Hz, 1H), 6.67 (d, J=8.4 Hz, 1H), 5.38 (s, 2H), 5.10-5.16 (m, 1H), 4.74-4.79 (m, 1H), 4.60-4.65 (m, 1H), 4.44-4.50 (m, 1H), 4.37-4.42 (m, 1H), 3.94 (d, J=13.6 Hz, 1H), 3.78 (d, J=13.6 Hz, 1H), 2.98-3.01 (m, 1H), 2.84-2.87 (m, 1H), 2.58-2.74 (m, 2H), 2.41-2.46 (m, 1H), 2.15-2.28 (m, 2H), 1.66-1.82 (m, 4H).

### Example 87: 2-[(4-{6-[(4-chloro-2-fluorophenyl)methoxy]pyridin-2-yl}piperidin-1-yl)methyl]-N-cyclopropyl-1-{[(2S)-oxetan-2-yl]methyl}-1H-1,3-benzodiazole-5-carboxamide (Compound 396a)

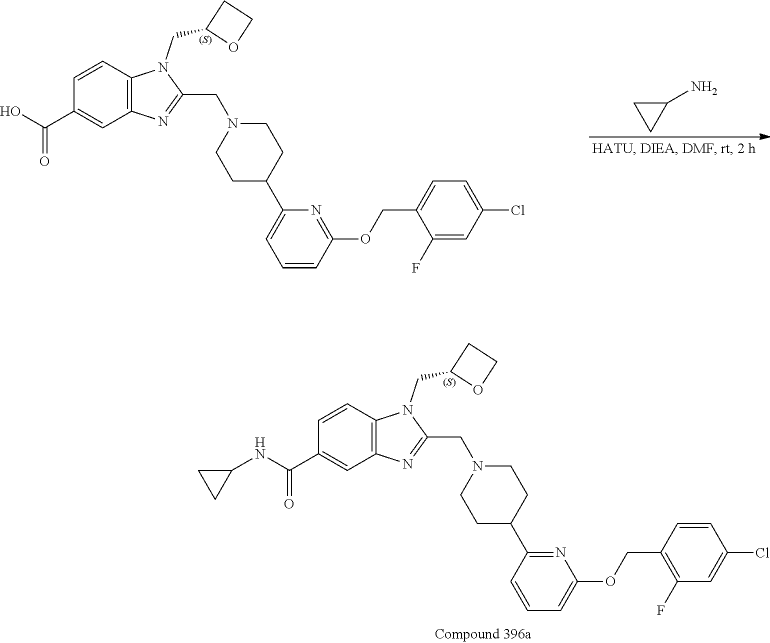

To a solution of 2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazole-5-carboxylic acid (45 mg, 0.080 mmol) in DMF (1 ml) was added cyclopropanamine (5.4 mg, 0.096 mmol), HATU (46 mg, 0.120 mmol) and DIEA (20.6 mg, 0.160 mmol). The resulting mixture was stirred at room temperature for 2 hours. The reaction was diluted with water, extracted with ethyl acetate. The combined organic extracts were dried (Na₂SO₄), filtered and concentrated under vacuum. The residue was purified by Prep-HPLC to give Compound 396a (2.90 mg, yield: 6%) as a white solid.

MS Calcd.: 603.2; MS Found: 604.1 [M+H]⁺.

¹H NMR (400 MHz, DMSO-d₆): 8.37 (d, J=4.4 Hz, 1H), 8.09-8.10 (m, 1H), 7.73 (dd, J=8.4 Hz, 1.2 Hz, 1H), 7.61-7.67 (m, 2H), 7.56 (t, J=8.0 Hz, 1H), 7.46 (dd, J=10.0 Hz, 2.0 Hz, 1H), 7.29 (dd, J=8.0 Hz, 1.6 Hz, 1H), 6.87 (d, J=7.2 Hz, 1H), 6.67 (d, J=8.4 Hz, 1H), 5.38 (s, 2H), 5.09-5.15 (m, 1H), 4.70-4.78 (m, 1H), 4.59-4.64 (m, 1H), 4.42-4.49 (m, 1H), 4.35-4.41 (m, 1H), 3.92 (d, J=13.6 Hz, 1H), 3.78 (d, J=13.2 Hz, 1H), 2.99-3.02 (m, 1H), 2.82-2.89 (m, 2H), 2.56-2.73 (m, 2H), 2.38-2.47 (m, 1H), 2.12-2.26 (m, 2H), 1.62-1.82 (m, 4H), 0.67-0.72 (m, 2H), 0.57-0.60 (m, 2H).

### Example 88: 3-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-1H-pyrazole-4-carboxylic acid (Compound 214a)

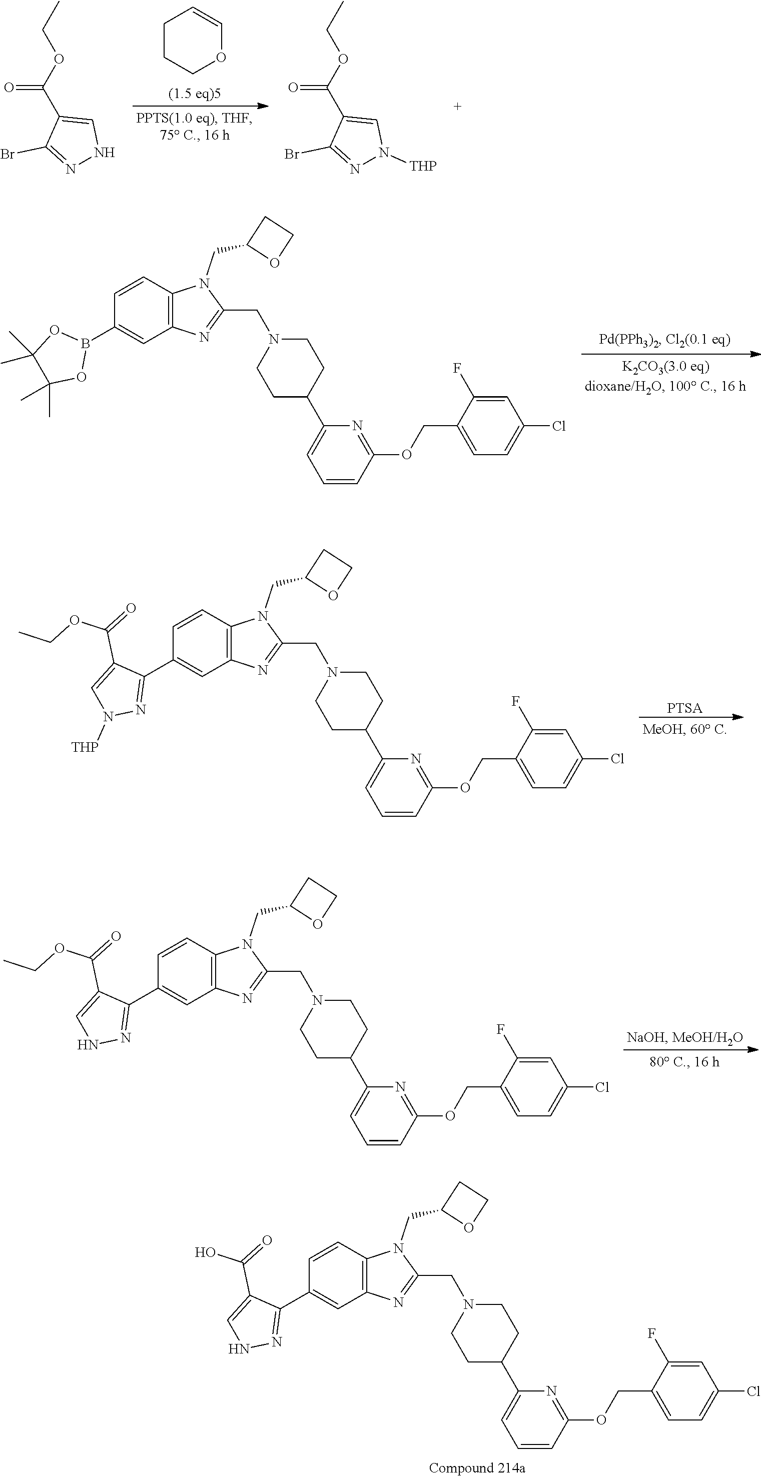

### Step A: ethyl 3-bromo-1-(tetrahydro-2H-pyran-2-yl)-1H-pyrazole-4-carboxylate

A mixture of ethyl 3-bromo-1H-pyrazole-4-carboxylate (500 mg, 2.29 mmol) and 3,4-dihydro-2H-pyran (289 mg, 3.44 mmol) in THF (15 mL) was added PPTS (576 mg, 2.29 mmol) and stirred at 75° C. for 16 hours. The mixture was diluted with EtOAc (100 ml). The organic layer was washed with water and brine, dried over sodium sulfate, filtered and concentrated in vacuum to give ethyl 3-bromo-1-(tetrahydro-2H-pyran-2-yl)-1H-pyrazole-4-carboxylate (370 mg, 53% yield), which was used in next step without further purification.

MS Calcd.: 302.0; MS Found: 303.0 [M+H]⁺.

### Step B: ethyl 3-(2-((4-(6-((4-chloro-2-fluorobenzyl)oxy)pyridin-2-yl)piperidin-1-yl) methyl)-1-(((S)-oxetan-2-yl)methyl)-1H-benzo[d]imidazol-5-yl)-1-(tetrahydro-2H-pyran-2-yl)-1H-pyrazole-4-carboxylate

A mixture of (S)-2-((4-(6-((4-chloro-2-fluorobenzyl)oxy)pyridin-2-yl)piperidin-1-yl)methyl)-1-(oxetan-2-ylmethyl)-5-(4,4,5,5-tetramethyl-1,3,2-dioxaborolan-2-yl)-1H-benzo[d]imidazole (200 mg, 0.31 mmol), ethyl 3-bromo-1-(tetrahydro-2H-pyran-2-yl)-1H-pyrazole-4-carboxylate (280 mg), K₂CO₃ (130 mg, 0.93 mmol) and Pd(dppf)Cl₂ (21 mg, 0.029 mmol) in dioxane (4 mL) and H₂O (0.4 mL) was stirred at 100° C. under N₂ for 16 hours. The mixture was diluted with EtOAc (100 ml) and washed with water and brine. The organic layer was dried over sodium sulfate, filtered and concentrated in vacuum. The residue was purified by prep-TLC to give ethyl 3-(2-((4-(6-((4-chloro-2-fluorobenzyl)oxy)pyridin-2-yl)piperidin-1-yl)methyl)-1-(((S)-oxetan-2-yl)methyl)-1H-benzo[d]imidazol-5-yl)-1-(tetrahydro-2H-pyran-2-yl)-1H-pyrazole-4-carboxylate (130 mg, 56.6% yield).

MS Calcd.: 742.3; MS Found: 743.3 [M+H]⁺.

### Step C: ethyl (S)-3-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)-1H-pyrazole-4-carboxylate

A mixture of ethyl3-(2-((4-(6-((4-chloro-2-fluorobenzyl)oxy)pyridin-2-yl)piperidin-1-yl) methyl)-1-(((S)-oxetan-2-yl)methyl)-1H-benzo [d]imidazol-5-yl)-1-(tetrahydro-2H-pyran-2-yl)-1H-pyrazole-4-carboxylate (130 mg, 0.18 mmol) and PTSA (17.2 mg, 0.09 mmol) in MeOH (6 mL) was stirred at 60° C. for 3 hours. Then another portion of PTSA (17.2 mg, 0.09 mmol) was added and the mixture was stirred at 60° C. for an additional 4 hours. After cooled down to the room temperature, the mixture was diluted with EtOAc (90 ml) and washed with water and brine. The organic layer was dried over sodium sulfate, filtered and concentrated in vacuum to give ethyl (S)-3-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)-1H-pyrazole-4-carboxylate (80 mg, crude), which was used in next step without further purifications.

MS Calcd.: 658.2; MS Found: 659.2 [M+H]⁺.

### Step D: 3-{2-[(4-{6-[(4-chloro-2-fluorophenyl)methoxy]pyridin-2-yl}piperidin-1-yl)methyl]-1-{[(2S)-oxetan-2-yl]methyl}-1H-1,3-benzodiazol-5-yl}-1H-pyrazole-4-carboxylic acid (Compound 214a)

To a solution of ethyl (S)-3-(2-((4-(6-((4-chloro-2-fluorobenzyl)oxy)pyridin-2-yl)piperidin-1-yl)methyl)-1-(oxetan-2-ylmethyl)-1H-benzo[d]imidazol-5-yl)-1H-pyrazole-4-carboxylate (80 mg) in MeOH (4 mL) and H₂O (1.3 mL) was added NaOH (48.6 mg, 1.2 mmol). The reaction was stirred at 80° C. for 16 hours. After cooled down to room temperature, the pH value of the mixture was adjusted to 7 with aqueous HCl (2 mol/L), diluted with DCM (10 mL) and washed with water and brine. The organic layer was dried over sodium sulfate, filtered and concentrated in vacuum, the residue was purified by Prep-HPLC to give Compound 214a (16.3 mg, yield: 42.6%) as a white solid.

MS Calcd.: 630.2; MS Found: 631.2 [M+H]⁺.

¹H NMR (400 MHz, methanol-d₄): 8.09 (s, 1H), 8.02 (s, 1H), 7.66 (s, 2H), 7.57 (t, J=8.0 Hz, 1H), 7.49 (t, J=8.0 Hz, 1H), 7.23-7.17 (m, 2H), 6.82 (d, J=7.2 Hz, 1H), 6.63 (d, J=8.4 Hz, 1H), 5.41 (s, 2H), 5.32-5.25 (m, 1H), 4.84-4.82 (m, 1H), 4.71 (dd, J=15.2 Hz, J=13.2 Hz, 1H), 4.65-4.60 (m, 1H), 4.48-4.41 (m, 1H), 4.05 (d, J=13.6 Hz, 1H), 3.95 (d, J=13.6 Hz, 1H), 3.12-3.09 (m, 1H), 3.02-2.98 (m, 1H), 2.81-2.77 (m, 1H), 2.68-2.60 (m, 1H), 2.58-2.52 (m, 1H), 2.41-2.28 (m, 2H), 1.92-1.86 (m, 4H). ¹⁹F-NMR (400 MHz, methanol-d₄): δ −117.63.

### Example 89: 2-[(4-{6-[(4-chloro-2-fluorophenyl)methoxy]pyridin-2-yl}piperidin-1-yl)methyl]-1-{[(2S)-oxetan-2-yl]methyl}-5-(1H-pyrazol-5-yl)-1H-1,3-benzodiazole (Compound 215a)

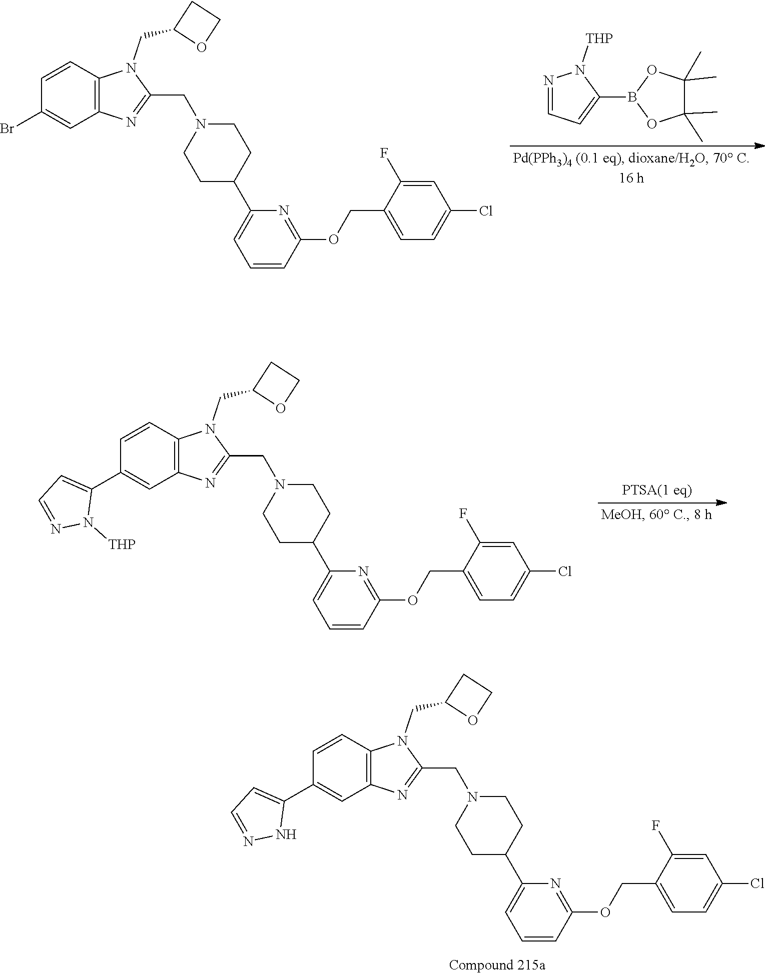

### Step A: 2-((4-(6-((4-chloro-2-fluorobenzyl)oxy)pyridin-2-yl)piperidin-1-yl)methyl)-1-((S)-oxetan-2-ylmethyl)-5-(1-(tetrahydro-2H-pyran-2-yl)-1H-pyrazol-5-v)-1H-benzo[d]imidazole

A mixture of (S)-5-bromo-2-((4-(6-((4-chloro-2-fluorobenzyl)oxy)pyridin-2-yl)piperidin-1-yl)methyl)-1-(oxetan-2-ylmethyl)-1H-benzo[d]imidazole (119 mg, 0.20 mmol) and 1-(tetrahydro-2H-pyran-2-yl)-5-(4,4,5,5-tetramethyl-1,3,2-dioxaborolan-2-yl)-1H-pyrazole (111 mg, 0.40 mmol), Pd(PPh₃)₄ (23 mg, 0.02 mmol) and K₂CO₃ (83 mg, 0.60 mmol) in dioxane (3.5 mL) and water (0.5 mL) was stirred at 70° C. under N₂ for 16 hours. The reaction mixture was concentrated in vacuum, purified by FCC (DCM/MeOH=100/1-50/1) to give 2-((4-(6-((4-chloro-2-fluorobenzyl)oxy)pyridin-2-yl)piperidin-1-yl)methyl)-1-((S)-oxetan-2-ylmethyl)-5-(1-(tetrahydro-2H-pyran-2-yl)-1H-pyrazol-5-yl)-1H-benzo[d]imidazole (100 mg, yield: 74.6%) as yellow oil.

MS Calcd.: 670.3; MS Found: 671.3 [M+H]⁺.

### Step B: 2-[(4-{6-[(4-chloro-2-fluorophenyl)methoxy]pyridin-2-yl}piperidin-1-yl)methyl]-1-{[(2S)-oxetan-2-yl]methyl}-5-(1H-pyrazol-5-yl)-1H-1,3-benzodiazole (Compound 215a)

To a solution of 2-((4-(6-((4-chloro-2-fluorobenzyl)oxy)pyridin-2-yl)piperidin-1-yl)methyl)-1-((S)-oxetan-2-ylmethyl)-5-(1-(tetrahydro-2H-pyran-2-yl)-1H-pyrazol-5-yl)-1H-benzo[d]imidazole (100 mg, 0.15 mmol) in methanol (4 mL) was added p-toluenesulfonic acid monohydrate (15 mg, 0.075 mmol), the mixture was stirred at 60° C. for 4 hours. Anther portion of p-toluenesulfonic acid monohydrate (15 mg, 0.075 mmol) was added, the mixture was stirred at 60° C. for another 4 hours. The reaction mixture was concentrated in vacuum, diluted with MeOH, purified by prep-HPLC (0.1% NH₄HCO₃) directly to give Compound 215a (47.8 mg, yield: 54.3%) as white solid.

MS Calcd.: 586.2; MS Found: 587.2 [M+H]⁺.

¹H NMR (400 MHz, methanol-d₄) δ 7.92 (br.s, 1H), 7.69-7.57 (m, 3H), 7.47 (t, J=7.6 Hz, 1H), 7.39 ((t, J=8.4 Hz, 1H), 7.13-7.07 (m, 2H), 6.73 (d, J=7.2 Hz, 1H), 6.58 (br.s, 1H), 6.54 (d, J=8.4 Hz, 1H), 5.32 (s, 2H), 5.21-5.15 (m, 1H), 4.76-4.72 (m, 1H), 4.63-4.58 (m, 1H), 4.56-4.50 (m, 1H), 4.40-4.34 (m, 1H), 3.91-3.76 (m, 2H), 2.97-2.95 (m, 1H), 2.88-2.83 (m, 1H), 2.74-2.64 (m, 1H), 2.58-2.40 (m, 2H), 2.25-2.13 (m, 2H), 1.81-1.72 (m, 4H).

¹⁹F NMR (400 MHz, methanol-d₄) δ −117.66.

### Example 90: 2-[(4-{6-[(4-chloro-2-fluorophenyl)methoxy]pyridin-2-yl}piperidin-1-yl)methyl]-5-[1-(oxan-3-yl)-1H-pyrazol-3-yl]-1-{[(2S)-oxetan-2-yl]methyl}-1H-1,3-benzodiazole (Compound 216a)

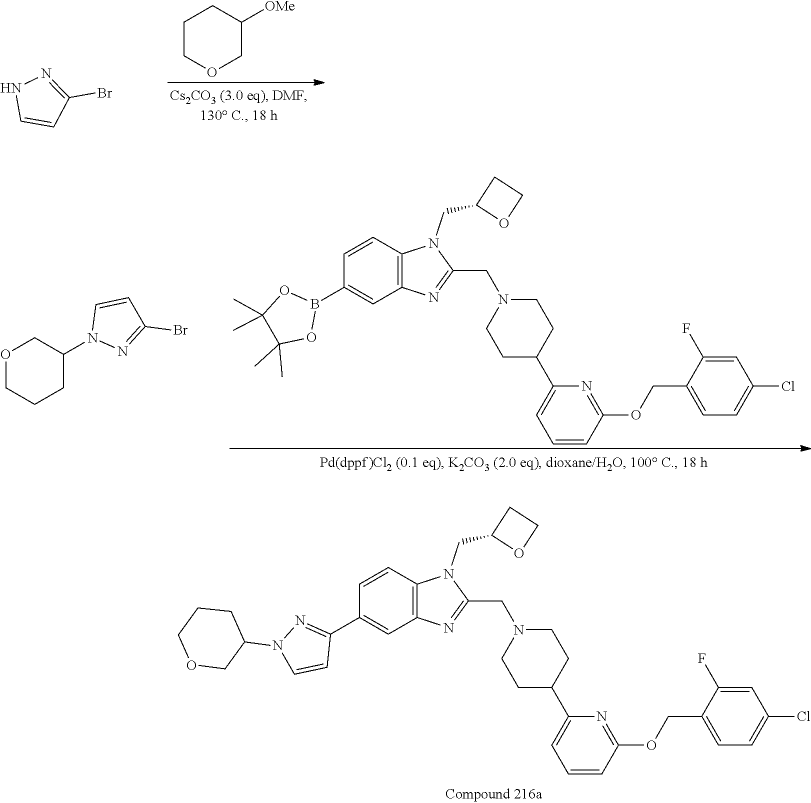 [SMILES: COC1CCCOC1 ; Brc1cc[nH]n1 ; Brc1ccn(C2CCCOC2)n1 ; CC1(C)OB(c2ccc3c(c2)nc(CN2CCC(c4cccc(OCc5ccc(Cl)cc5F)n4)CC2)n3C[C@@H]2CCO2)OC1(C)C ; Fc1cc(Cl)ccc1COc1cccc(C2CCN(Cc3nc4cc(-c5ccn(C6CCCOC6)n5)ccc4n3C[C@@H]3CCO3)CC2)n1]

### Step A: 3-bromo-1-(tetrahydro-2H-pyran-3-yl)-1H-pyrazole

A mixture of 3-bromo-1H-pyrazole (1.0 g, 6.85 mmol), tetrahydro-2H-pyran-3-yl methanesulfonate (1.2 g, 6.85 mmol), and cesium carbonate (3.7 g, 20.55 mmol) in DMF (50 ml) was stirred at 130° C. for 18 hours. After the reaction was completed, the mixture was extracted with ethyl acetate (15 ml×3), washed with brine (15 ml×2), dried over sodium sulfate, filtered and concentrated in vacuum, the residue was purified by column chromatography to give 3-bromo-1-(tetrahydro-2H-pyran-3-yl)-1H-pyrazole (180 mg, yield: 11.4%) as dark oil.

### Step B: 2-[(4-{6-[(4-chloro-2-fluorophenyl)methoxy]pyridin-2-yl}piperidin-1-yl)methyl]-5-[1-(oxan-3-yl)-1H-pyrazol-3-yl]-1-{[(2S)-oxetan-2-yl]methyl}-1H-1,3-benzodiazole (Compound 216a)

A mixture of (S)-2-((4-(6-((4-chloro-2-fluorobenzyl)oxy)pyridin-2-yl)piperidin-1-yl)methyl)-1-(oxetan-2-ylmethyl)-5-(4,4,5,5-tetramethyl-1,3,2-dioxaborolan-2-yl)-1H-benzo[d]imidazole (80 mg, 0.124 mmol), 3-bromo-1-(tetrahydro-2H-pyran-3-yl)-1H-pyrazole (85 mg, 0.372 mmol), Pd(dppf)Cl₂ (9 mg, 0.012 mmol) and K₂CO₃ (34 mg, 0.248 mmol) in dioxane/H₂O (5 mL/1.5 mL) was stirred at 100° C. under Ar for 18 hours. The mixture was poured into water (50 mL) and extracted with EtOAc (2×50 mL), the combined organic layer was concentrated under reduced pressure, the residue was purified by prep-HPLC to give Compound 216a (9.9 mg, yield: 11.9%) as white solid.

MS Calcd.: 670.3; MS Found: 671.2 [M+H]⁺.

¹H NMR (400 MHz, methanol-d₄) δ 8.01 (d, J=0.8 Hz, 1H), 7.78-7.75 (m, 2H), 7.63 (d, J=8.8 Hz, 1H), 7.57 (t, J=8.4 Hz, 1H), 7.49 (t, J=8.0 Hz, 1H), 7.23-7.17 (m, 2H), 6.82 (d, J=7.2 Hz, 1H), 6.65-6.62 (m, 2H), 5.42 (s, 2H), 5.29-5.26 (m, 1H), 4.85-4.81 (m, 1H), 4.72-4.60 (m, 2H), 4.47-4.44 (m, 1H), 4.39-4.35 (m, 1H), 4.23-4.09 (m, 1H), 4.01-3.97 (m, 1H), 3.91-3.86 (m, 2H), 3.81-3.75 (m, 1H), 3.61-3.55 (m, 1H), 3.06-3.05 (m, 1H), 2.98-2.92 (m, 1H), 2.82-2.73 (m, 1H), 2.70-2.60 (m, 1H), 2.60-2.50 (m, 1H), 2.34-2.17 (m, 4H), 1.90-1.80 (m, 6H).

### Example 91: 2-[(4-{6-[(4-chloro-2-fluorophenyl)methoxy]pyridin-2-yl}piperidin-1-yl)methyl]-1-{[(2S)-oxetan-2-yl]methyl}-5-[(E)-2-(pyridin-3-yl)ethenyl]-1H-1,3-benzodiazole (Compound 217a)

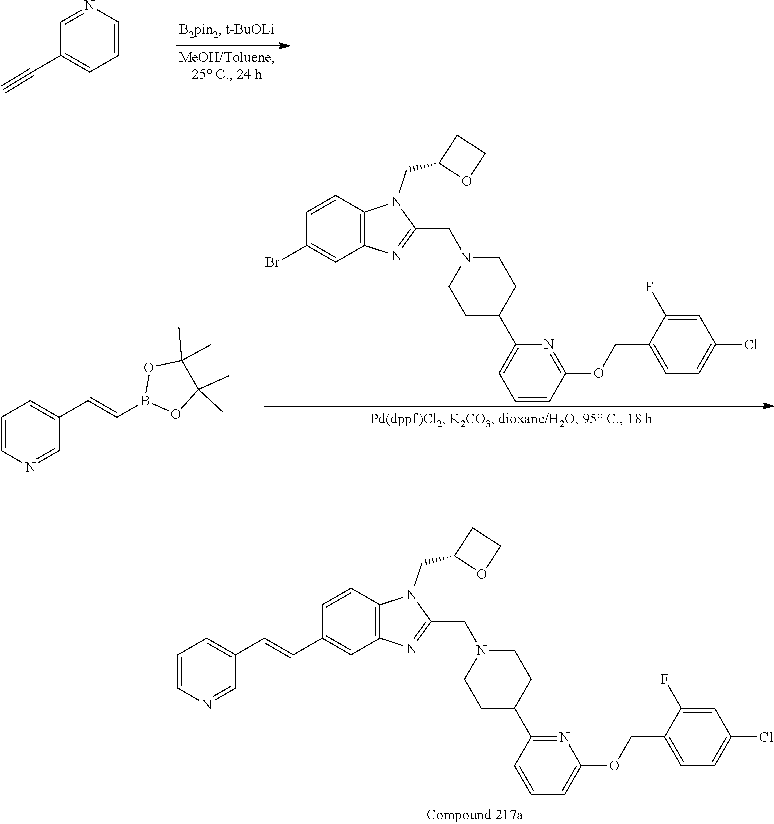

### Step A: (E)-3-(2-(4,4,5,5-tetramethyl-1,3,2-dioxaborolan-2-yl)vinyl)pyridine

A mixture of 3-ethynylpyridine (1.0 g, 9.71 mmol), B₂Pin₂ (3.7 mg, 14.56 mmol) in methanol (20 mL) and toluene (1.0 mL) was added lithium tert-butoxide (388 mg, 4.85 mmol) under N₂. The mixture was stirred vigorously at room temperature for 24 h. The mixture was then cooled to room temperature and concentrated under reduced pressure. The residue was purified by silica column chromatography to give (E)-3-(2-(4,4,5,5-tetramethyl-1,3,2-dioxaborolan-2-yl)vinyl)pyridine (500 mg, yield: 22.3%) as dark oil.

MS Calcd.: 231.1; MS Found: 232.1 [M+H]⁺.

### Step B: 2-[(4-{6-[(4-chloro-2-fluorophenyl)methoxy]pyridin-2-yl}piperidin-1-yl)methyl]-1-{[(2S)-oxetan-2-yl]methyl}-5-[(E)-2-(pyridin-3-yl)ethenyl]-1H-1,3-benzodiazole (Compound 217a)

A mixture of (S)-5-bromo-2-((4-(6-((4-chloro-2-fluorobenzyl)oxy)pyridin-2-yl)piperidin-1-yl)methyl)-1-(oxetan-2-ylmethyl)-11H-benzo[d]imidazole (60 mg, 0.093 mmol), (E)-3-(2-(4,4,5,5-tetramethyl-1,3,2-dioxaborolan-2-yl)vinyl)pyridine (64 mg, 0.279 mmol), Pd(dppf)Cl₂ (7 mg, 0.009 mmol) and K₂CO₃ (39 mg, 0.279 mmol) in dioxane/H₂O (5 mL/0.5 mL) was stirred at 95° C. under Ar for 18 hours. The mixture was poured into water (20 mL) and extracted with EtOAc (2×50 mL), the combined organic layer was concentrated under reduced pressure, the residue was purified by prep-HPLC to give Compound 217a (16.1 mg, yield: 25.8%) as white solid.

MS Calcd.: 623.2; MS Found: 624.2 [M+H]⁺.

¹H NMR (400 MHz, methanol-d₄) δ8.70 (d, J=1.2 Hz, 1H), 8.38 (d, J=4.0 Hz, 1H), 8.10 (d, J=8.0 Hz, 1H), 7.83 (s, 1H), 7.63 (s, 2H), 7.57 (t, J=8.0 Hz, 1H), 7.51-7.41 (m, 3H), 7.24-7.17 (m, 3H), 6.82 (d, J=7.2 Hz, 1H), 6.62 (d, J=8.4 Hz, 1H), 5.41 (s, 2H), 5.30-5.22 (m, 1H), 4.87-4.81 (m, 1H), 4.73-4.60 (m, 2H), 4.49-4.42 (m, 1H), 3.98 (d, J=13.2 Hz, 1H), 3.87 (d, J=13.6 Hz, 1H), 3.09-3.02 (m, 1H), 2.96-2.93 (m, 1H), 2.82-2.78 (m, 1H), 2.69-2.60 (m, 1H), 2.60-2.51 (m, 1H), 2.38-2.22 (m, 2H), 1.90-1.82 (m, 4H).

¹⁹F NMR (400 MHz, methanol-d₄) δ −117.65.

### Example 92: 4-[2-(4-chloro-2-fluorophenyl)-2-methyl-2H-1,3-benzodioxol-4-yl]-1-[(3-{[(2S)-oxetan-2-yl]methyl}-6-(1H-1,2,3,4-tetrazol-5-yl)-3H-imidazo[4,5-c]pyridin-2-yl)methyl]piperidine (Compound 218a)

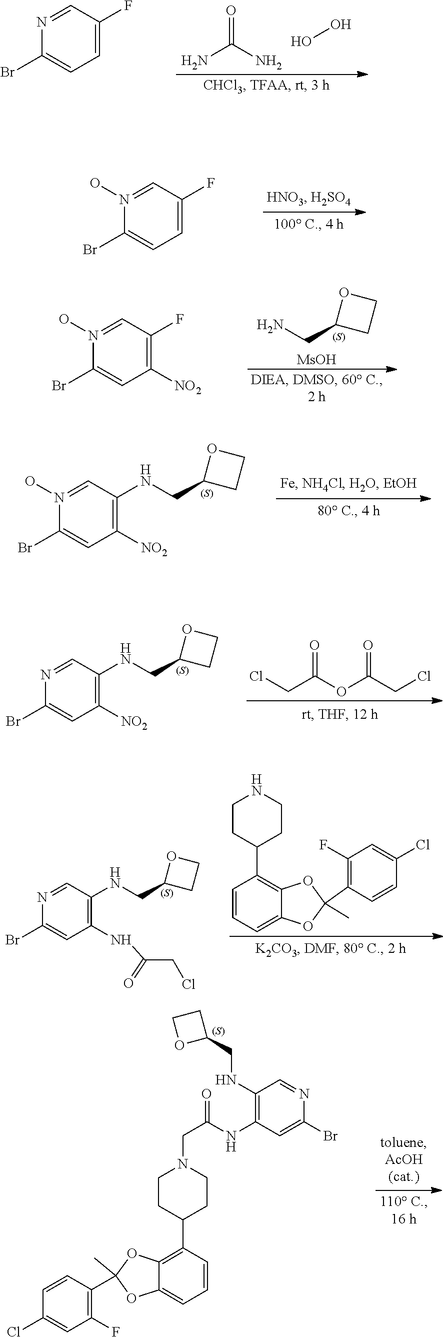

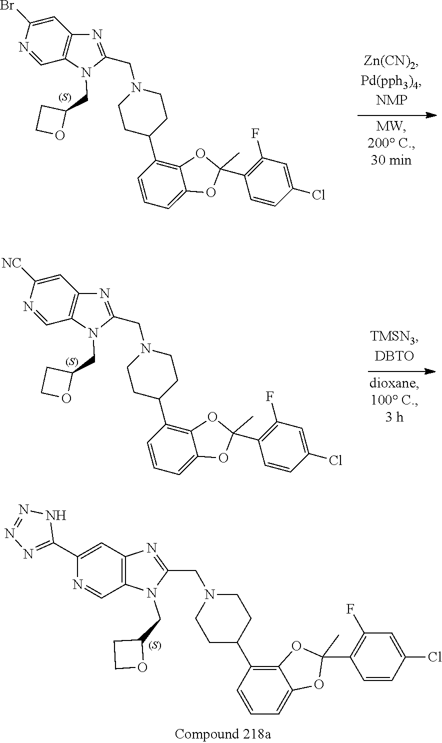

### Step A: 2-bromo-5-fluoropyridine 1-oxide

To a solution of 2-bromo-5-fluoropyridine (5 g, 28.4 mmol), Urea peroxide (3.47 g, 36.9 mmol) in CHCl₃ (50 mL) was added TFAA (11.9 g, 56.8 mmol) at 0° C. The reaction mixture was heated to room temperature for 3 hours. The reaction was quenched with NaHSO₃, extracted with ethyl acetate. The organic layers were combined, dried over Na₂SO₄, filtered and concentrated under vacuum. The residue was purified by silica gel column and eluted with PE:EtOAc=10:1 to give 2-bromo-5-fluoropyridine 1-oxide (4.9 mg, 90.7% yield) as a white solid.

MS Calcd.: 190.9; MS Found: 191.8 [M+H]⁺.

### Step B: 2-bromo-5-fluoro-4-nitropyridine 1-oxide

To a solution of 2-bromo-5-fluoropyridine 1-oxide (3 g, 15.7 mmol) in H₂SO₄ (52 mL) was added HNO₃ (26 mL) at room temperature. The reaction mixture was heated to 100° C. for 4 hours. The reaction was poured into ice water slowly, pH value was adjusted to 7 with Na₂CO₃ and extracted with EA. The organic layers were combined, dried over Na₂SO₄, filtered and concentrated under vacuum. The residue was purified by silica gel column and eluted with PE:EtOAc=5:1 to give 2-bromo-5-fluoro-4-nitropyridine 1-oxide (3 g, 93% yield) as a yellow solid.

### Step C: (S)-2-bromo-4-nitro-5-((oxetan-2-ylmethyl)amino)pyridine 1-oxide

To a solution of 2-bromo-5-fluoro-4-nitropyridine 1-oxide (500 mg, 2.118 mmol) in DMSO (8 mL) was added (S)-oxetan-2-ylmethanamine methanesulfonate (465 mg, 2.542 mmol) and DIEA (546 mg, 4.236 mmol). The reaction mixture was stirred at 60° C. for 2 hours. The reaction was quenched with water, extracted with ethyl acetate. The organic layers were combined, dried over Na₂SO₄, filtered and concentrated under vacuum. The residue was purified by silica gel column and eluted with PE:EtOAc=1:1 to give (S)-2-bromo-4-nitro-5-((oxetan-2-ylmethyl)amino)pyridine 1-oxide (510 mg, 79% yield) as a yellow solid.

MS Calcd.: 303.0; MS Found: 303.9 [M+H]⁺.

### Step D: (S)-6-bromo-N³-(oxetan-2-ylmethyl)pyridine-3,4-diamine

To a solution of (S)-2-bromo-4-nitro-5-((oxetan-2-ylmethyl)amino)pyridine 1-oxide (510 mg, 1.650 mmol) in EtOH (10 mL) and water (2 mL) was added ammonium chloride (875 mg, 16.50 mmol) and iron powder (924 mg, 16.50 mmol). The solution was stirred at 80° C. for 4 hours. The reaction mixture was filtered through a celite pad. The filtrate was concentrated under vacuum. The residue was washed with water and ethyl acetate. The organic layer was dried over Na₂SO₄, filtered and concentrated under vacuum to give (S)-6-bromo-N³-(oxetan-2-ylmethyl)pyridine-3,4-diamine (300 mg, 70.7% yield).

MS Calcd.: 257.0; MS Found: 299.0 [M+H+41]⁺.

### Step E: (S)—N-(2-bromo-5-((oxetan-2-ylmethyl)amino)pyridin-4-yl)-2-chloroacetamide

To a solution of (S)-6-bromo-N3-(oxetan-2-ylmethyl)pyridine-3,4-diamine (600 mg, 2.334 mmol) in THF (10 mL) was added 2-chloroacetic anhydride (439 mg, 2.568 mmol). The resulting mixture was stirred at room temperature for 12 hours. The solvent was removed in vacuo. The residue was purified by silica gel column and eluted with PE:EtOAc=1:2 to give (S)—N-(2-bromo-5-((oxetan-2-ylmethyl)amino)pyridin-4-yl)-2-chloroacetamide (500 mg, 63.9% yield) as a white solid.

MS Calcd.: 333.0; MS Found: 334.0 [M+H]⁺.

### Step F: N-(2-bromo-5-((((S)-oxetan-2-yl)methyl)amino)pyridin-4-yl)-2-(4-(2-(4-chloro-2-fluorophenyl)-2-methylbenzo[d][1,3]dioxol-4-yl)piperidin-1-yl)acetamide

A mixture of (S)—N-(2-bromo-5-((oxetan-2-ylmethyl)amino)pyridin-4-yl)-2-chloroacetamide (500 mg, 1.492 mmol), 4-(2-(4-chloro-2-fluorophenyl)-2-methylbenzo[d][1,3]dioxol-4-yl)piperidine (573 mg) and K₂CO₃ (618 mg, 4.476 mmol) in DMF (10 mL) was heated to 80° C. for 2 hours. The reaction was quenched with water, extracted with ethyl acetate. The organic layers were combined, dried over Na₂SO₄, filtered and concentrated under vacuum. The residue was purified by silica gel column and eluted with PE:EtOAc=1:2 to give N-(2-bromo-5-((((S)-oxetan-2-yl)methyl)amino)pyridin-4-yl)-2-(4-(2-(4-chloro-2-fluorophenyl)-2-methylbenzo[d][1,3]dioxol-4-yl)piperidin-1-yl)acetamide (290 mg, 30% yield) as a white solid.

MS Calcd.: 644.1; MS Found: 645.1 [M+H]⁺.

### Step G: 6-bromo-2-((4-(2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine

A mixture of N-(2-bromo-5-((((S)-oxetan-2-yl)methyl)amino)pyridin-4-yl)-2-(4-(2-(4-chloro-2-fluorophenyl)-2-methylbenzo[d][1,3]dioxol-4-yl)piperidin-1-yl)acetamide (260 mg, 0.366 mmol) and AcOH (1 drop) in toluene (5 mL) was stirred at 110° C. for 16 hours. The solvent was concentrated under vacuum. The residue was purified by silica gel column and eluted with EtOAc to give 6-bromo-2-((4-(2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine (180 mg, 71% yield) as a light yellow solid.

MS Calcd.: 626.1; MS Found: 627.1 [M+H]⁺.

### Step H: 2-((4-(2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile

A degassed solution of 6-bromo-2-((4-(2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine (90 mg, 0.143 mmol), Zn(CN)₂ (12.6 mg, 0.107 mmol) and Pd(PPh₃)₄ (24.8 mg, 0.021 mmol) in anhydrous NMP (1.5 mL) was heated in a microwave oven at 200° C. for 30 minutes, then water was added. The mixture was extracted with ethyl acetate. The organic layers were combined, dried over Na₂SO₄, filtered and concentrated under vacuum. The residue was purified by Prep-TLC (DCM/MeOH=30/1) to give 2-((4-(2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (30 mg, yield: 36%) as an off-white solid.

MS Calcd.: 573.2; MS Found: 574.2 [M+H]⁺.

### Step I: 4-[2-(4-chloro-2-fluorophenyl)-2-methyl-2H-1,3-benzodioxol-4-yl]-1-[(3-{[(2S)-oxetan-2-yl]methyl}-6-(1H-1,2,3,4-tetrazol-5-yl)-3H-imidazo[4,5-c]pyridin-2-yl)methyl]piperidine (Compound 218a)

A mixture of 2-((4-(2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (30 mg, 0.052 mmol), TMSN₃ (18 mg, 0.157 mmol) and DBTO (dibutyl(oxo)tin) (26 mg, 0.104 mmol) in dioxane (2 mL) was heated to 100° C. for 3 hours under nitrogen atmosphere. The solvent was removed in vacuo. The crude product was purified by Prep-HPLC to afford Compound 218a (3.25 mg, yield: 10%) as a white solid.

MS Calcd.: 616.2; MS Found: 617.2 [M+H]⁺.

¹H NMR (400 MHz, DMSO-d₆): 9.20 (br.s, 1H), 8.38 (s, 1H), 7.52-7.60 (m, 2H), 7.34 (d, J=8.8 Hz, 1H), 6.70-6.81 (m, 3H), 5.15-5.22 (m, 1H), 4.86-4.96 (m, 1H), 4.72-4.79 (m, 1H), 4.39-4.53 (m, 2H), 4.02 (d, J=12.4 Hz, 1H), 3.87 (d, J=13.6 Hz, 1H), 3.03-3.09 (m, 1H), 2.88-2.92 (m, 1H), 2.62-2.80 (m, 2H), 2.39-2.46 (m, 1H), 2.18-2.33 (m, 2H), 2.03 (s, 3H), 1.68-1.82 (m, 4H).

### Example 93: 5-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-2,3-dihydro-1,3,4-oxadiazol-2-one (Compound 219a)

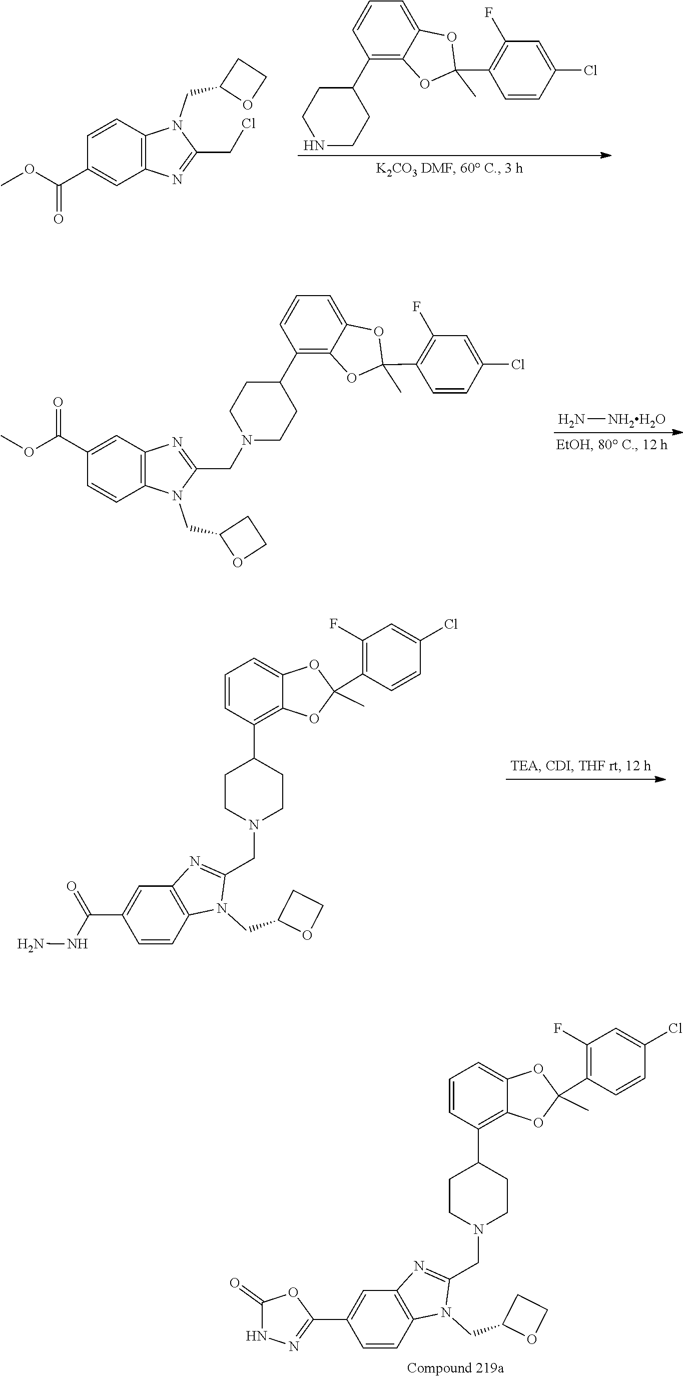

### Step A: methyl 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carboxylate

To a mixture of 4-(2-(4-chloro-2-fluorophenyl)-2-methylbenzo[d][1,3]dioxol-4-yl)piperidine (808 mg, 2.33 mmol) and K₂CO₃ (965 mg, 6.99 mmol) in DMF (7 ml) was added methyl (S)-2-(chloromethyl)-1-(oxetan-2-ylmethyl)-1H-benzo[d]imidazole-5-carboxylate (685 mg, 2.33 mmol). The reaction was stirred at 60° C. for 3 hours. The reaction was quenched with H₂O (10 mL) and extracted with EA (10 mL×3). The organic layers were combined, dried over Na₂SO₄ and concentrated. The residue was purified by silica gel column chromatography (PE/EA=1/1) to give methyl 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carboxylate (700 mg, yield: 86%) as yellow oil.

MS Calcd.: 605.2; MS Found: 606.3 [M+H]⁺.

### Step B: 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbohydrazide

To a solution of methyl 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carboxylate (100 mg, 0.17 mmol) in EtOH (1 mL) was added hydrazine hydrate (25 mg, 0.5 mmol). The reaction was stirred at 80° C. for 12 hours. After the reaction was completed, the reaction was quenched with H₂O (2 mL) and extracted with EA (2 mL×3). The organic layers were combined and concentrated to give 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbohydrazide (100 mg, yield: 80%) as colourless oil.

MS Calcd.: 605.2; MS Found: 606.0 [M+H]⁺.

### Step C: 5-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-2,3-dihydro-1,3,4-oxadiazol-2-one (Compound 219a)

To a solution of 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbohydrazide (100 mg, 0.17 mmol) in THE (1 mL) was added CDI (40 mg, 0.25 mmol) and TEA (35 mg, 0.34 mmol) at room temperature. The reaction mixture was stirred at rt for 12 hours. After the reaction was completed, the reaction was purified by Prep-HPLC to give Compound 219a (14.32 mg, yield: 14%) as a white solid.

MS Calcd.: 631.2; MS Found: 632.1 [M+H]⁺.

¹H NMR (400 MHz, DMSO-d₆): δ 8.20 (s, 1H), 7.97 (d, J=1.2 Hz, 1H), 7.80 (d, J=8.4 Hz, 1H), 7.68 (dd, J=8.4, 1.6 Hz, 1H), 7.52-7.59 (m, 2H), 7.34 (dd, J=8.4 Hz, 1.6 Hz, 1H), 6.72-6.81 (m, 3H), 5.09-5.14 (m, 1H), 4.72-4.80 (m, 1H), 4.61-4.65 (m, 1H), 4.35-4.51 (m, 2H), 3.92-3.97 (m, 1H), 3.79 (d, J=13.2 Hz, 1H), 2.98-3.03 (m, 1H), 2.84-2.90 (m, 1H), 2.60-2.74 (m, 2H), 2.40-2.51 (m, 1H), 2.12-2.30 (m, 2H), 2.02 (s, 3H), 1.68-1.80 (m, 4H).

### Example 94: 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carboxylic acid (Compound 161a)

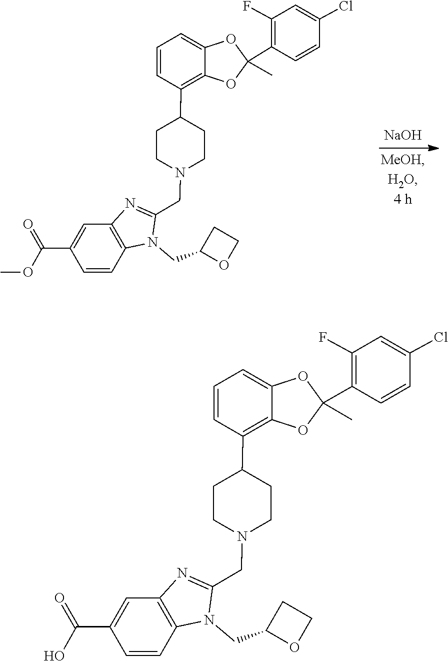 [SMILES: COC(=O)c1ccc2c(c1)nc(CN1CCC(c3cccc4c3OC(C)(c3ccc(Cl)cc3F)O4)CC1)n2C[C@@H]1CCO1 ; "NaOH" ; CC1(c2ccc(Cl)cc2F)Oc2cccc(C3CCN(Cc4nc5cc(C(=O)O)ccc5n4C[C@@H]4CCO4)CC3)c2O1]

To a solution of methyl 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carboxylate (100 mg, 0.16 mmol) in MeOH (6 mL) and H₂O (2 mL) was added NaOH (33 mg, 0.86 mmol) at room temperature. The reaction was stirred at 40° C. for 4 hours. After the reaction was completed, the reaction was quenched with water (20 mL) and extracted with ethyl acetate (20 mL×2). The organic layers were combined and washed with brine (10 mL×2), dried over sodium sulfate, filtered and concentrated in vacuum to give 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carboxylic acid (65 mg, 63% yield) as a white solid.

¹H NMR (400 MHz, CDCl₃) δ 8.60 (s, 1H), 8.09 (d, J=8.4 Hz, 1H), 7.45-7.63 (m, 2H), 7.06-7.17 (m, 2H), 6.65-6.80 (m, 3H), 5.19-5.33 (m, 1H), 4.72 (br.s, 2H), 4.60-4.66 (m, 1H), 4.34-4.47 (m, 1H), 3.90-4.16 (m, 2H), 2.90-3.09 (m, 2H), 2.68-2.81 (m, 2H), 2.26-2.60 (m, 3H), 2.04 (s, 3H), 1.75-1.20 (m, 4H).

MS Calcd.: 591.2; MS Found: 592.2 [M+H]⁺.

### Example 95: 5-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-3-methyl-2,3-dihydro-1,3,4-oxadiazol-2-one (Compound 220a)

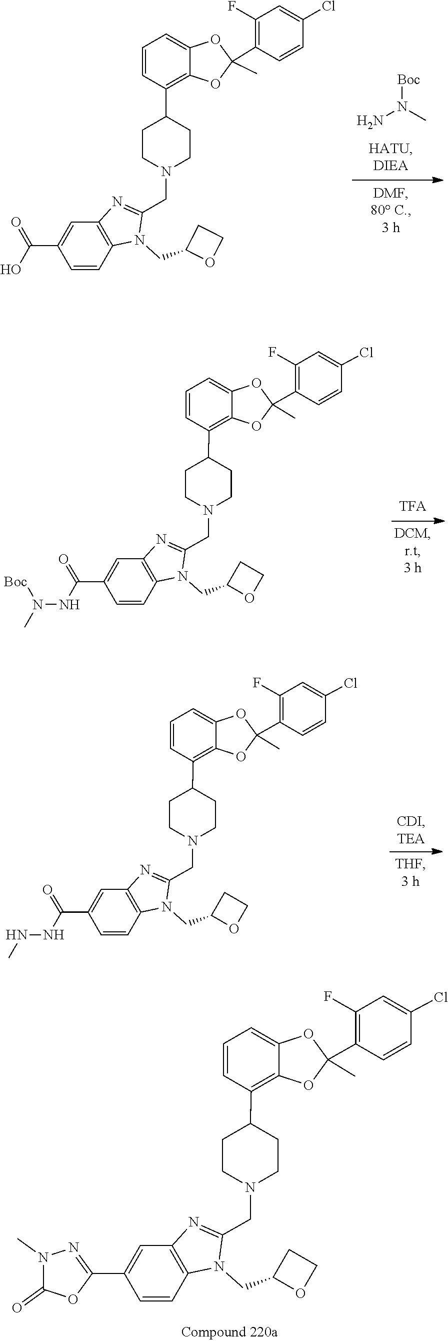

### Step A: tert-butyl 2-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonyl)-1-methylhydrazine-1-carboxylate

To a solution of 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carboxylic acid (65 mg, 0.11 mmol) in DMF (5 mL) was added tert-butyl 1-methylhydrazine-1-carboxylate (23.36 mg, 0.16 mmol), DIEA (35 mg, 0.27 mmol) and HATU (60 mg, 0.16 mmol). The reaction was stirred at 80° C. for 3 h. After the reaction was completed, the pH value of reaction mixture was adjusted to 7 with AcOH, followed by addition of water (20 mL) and extraction with ethyl acetate (20 mL×2). The organic layers were combined and washed with brine (10 mL×2), dried over sodium sulfate, filtered and concentrated in vacuum. The reaction was purified by prep-TLC to give tert-butyl 2-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonyl)-1-methylhydrazine-1-carboxylate (49 mg, 62% yield) as colorless oil.

MS Calcd.: 719.3; MS Found: 720.3 [M+H]⁺.

### Step B: 2-((4-(2-(4-chloro-2-fluorophenyl)-2-methylbenzo[d][1,3]dioxol-4-yl)piperidin-1-yl)methyl)-N′-methyl-1-(((S)-oxetan-2-yl)methyl)-1H-benzo[d]imidazole-5-carbohydrazide

A mixture of tert-butyl 2-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonyl)-1-methylhydrazine-1-carboxylate (49 mg, 0.22 mmol), TFA (0.125 mL) in DCM (0.5 ml) was stirred at rt for 3 h. The reaction was quenched with NaHCO₃ (10 mL) and extracted with ethyl acetate (20 mL×2). The organic layers were combined and dried over sodium sulfate, filtered and concentrated in vacuum to give 2-((4-(2-(4-chloro-2-fluorophenyl)-2-methylbenzo[d][1,3]dioxol-4-yl)piperidin-1-yl)methyl)-N′-methyl-1-(((S)-oxetan-2-yl)methyl)-1H-benzo[d]imidazole-5-carbohydrazide (57 mg, yield: 90%) as yellow oil.

MS Calcd.: 619.2; MS Found: 620.2 [M+H]⁺.

### Step C: 5-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-3-methyl-2,3-dihydro-1,3,4-oxadiazol-2-one (Compound 220a)

To a solution of 2-((4-(2-(4-chloro-2-fluorophenyl)-2-methylbenzo[d][1,3]dioxol-4-yl)piperidin-1-yl)methyl)-N′-methyl-1-(((S)-oxetan-2-yl)methyl)-1H-benzo[d]imidazole-5-carbohydrazide (57 mg, 0.092 mmol) in THE (0.5 mL) was added TEA (18 mg, 0.184 mmol) and CDI (22.3 mg, 0.138 mmol). The reaction was stirred at 30° C. for 3 h. After the reaction was completed, the reaction was purified by Prep-HPLC to give Compound 220a (1.36 mg, yield: 2%) as a white solid.

MS Calcd.: 645.2; MS Found: 646.3 [M+H]⁺.

¹H NMR (400 MHz, CDCl₃): δ: 8.27 (br.s, 1H), 7.89 (d, J=7.2 Hz, 1H), 7.47-7.54 (m, 2H), 7.11-7.14 (m, 2H), 6.72-6.82 (m, 2H), 6.68 (d, J=7.6 Hz, 1H), 4.44-5.27 (m, 6H), 3.72-4.02 (m, 2H), 3.53 (s, 3H), 3.28-3.44 (m, 2H), 2.70-3.05 (m, 3H), 2.30-2.60 (m, 3H), 2.01-2.20 (m, 5H).

### Example 96: 5-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-2,3-dihydro-1,3,4-thiadiazol-2-one (Compound 221a)

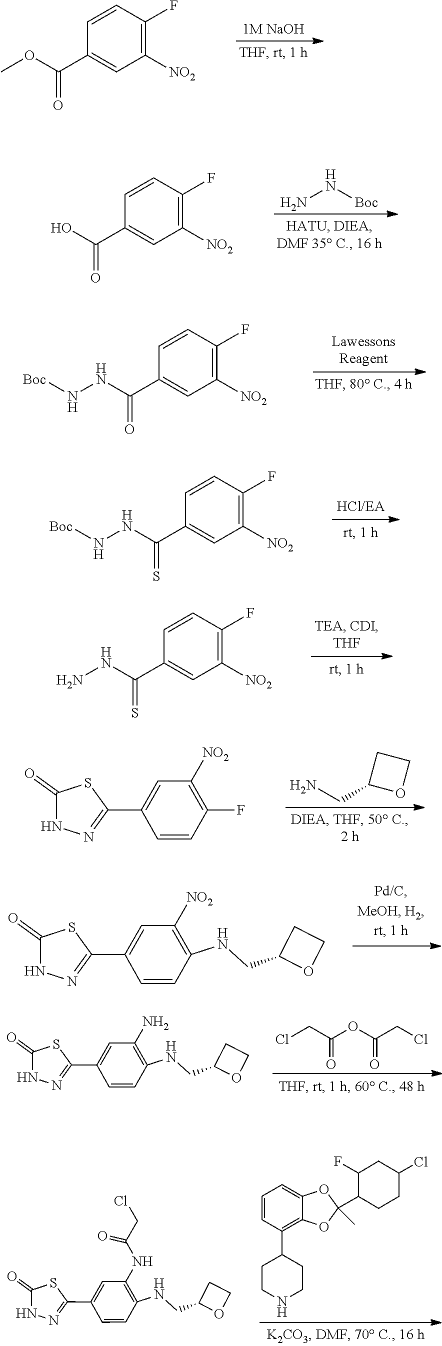

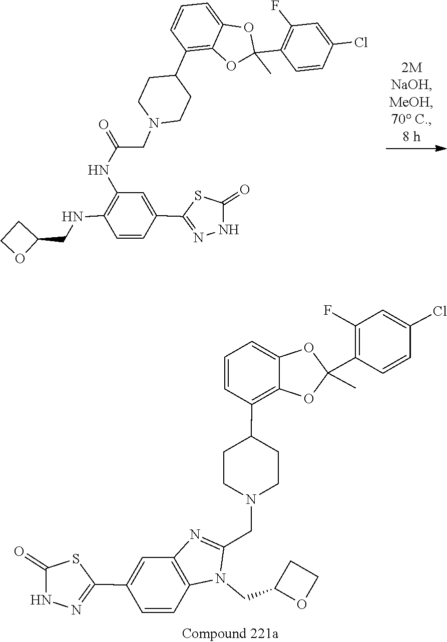 [SMILES: CC1(c2ccc(Cl)cc2F)Oc2cccc(C3CCN(CC(=O)Nc4cc(-c5n[nH]c(=O)s5)ccc4NC[C@@H]4CCO4)CC3)c2O1 ; CC1(c2ccc(Cl)cc2F)Oc2cccc(C3CCN(Cc4nc5cc(-c6n[nH]c(=O)s6)ccc5n4C[C@@H]4CCO4)CC3)c2O1]

### Step A: 4-fluoro-3-nitrobenzoic acid

To a solution of methyl 4-fluoro-3-nitrobenzoate (5 g, 25.13 mmol) in THF (50 mL) was added NaOH (1 M, 50 mL). The resulting mixture was stirred at room temperature for 1 hour. After the reaction was completed, the reaction mixture was concentrated under vacuum. The residue was diluted with water, and then pH value was adjusted to 4-5 using 1 N HCl. 200 mL of water was added, and the mixture was extracted with DCM (300 mL×3). The organic layer was combined and washed with brine (500 mL×2), dried over sodium sulfate, filtered and concentrated in vacuum to give 4-fluoro-3-nitrobenzoic acid (4.6 g, yield: 99%) as a yellow solid.

MS Calcd.: 185.0; MS Found: 183.8 [M−H]⁻.

### Step B: tert-butyl 2-(4-fluoro-3-nitrobenzoyl)hydrazine-1-carboxylate

A mixture of 4-fluoro-3-nitrobenzoic acid (4.6 g, 24.86 mmol), HATU (14.17 g, 37.29 mmol), DIEA (6.4 g, 49.72 mmol) and tert-butyl hydrazinecarboxylate (3.93 g, 29.80 mmol) in DMF (35 mL) was stirred at 35° C. for 16 hours. After the reaction was completed, the mixture was quenched with water (150 mL) and extracted with ethyl acetate (200 mL×3). The organic layer was combined and dried with Na₂SO₄. Then the mixture was filtered and concentrated in vacuum to give a residue, which was purified by column chromatography (PE:EA=6:1) to give tert-butyl 2-(4-fluoro-3-nitrobenzoyl)hydrazine-1-carboxylate (4.68 g, yield: 63%) as a yellow solid.

MS Calcd.: 299.1; MS Found: 297.8 [M−H]⁻.

### Step C: tert-butyl 2-(4-fluoro-3-nitrophenylcarbonothioyl)hydrazine-1-carboxylate

A mixture of tert-butyl 2-(4-fluoro-3-nitrobenzoyl)hydrazine-1-carboxylate (4.68 g, 15.65 mmol), Lawesson's Reagent (9.5 g, 23.48 mmol) in THE (50 mL) was stirred at 80° C. for 4 hours under N₂ atmosphere. After the reaction was completed, the reaction mixture was concentrated and the residue was purified by column chromatography (PE:EA=1:1) to give tert-butyl 2-(4-fluoro-3-nitrophenylcarbonothioyl) hydrazine-1-carboxylate (2.46 g, yield: 50%) as a green solid.

MS Calcd.: 315.1; MS Found: 314.1 [M−H]⁻.

### Step D: 4-fluoro-3-nitrobenzothiohydrazide

A mixture of tert-butyl 2-(4-fluoro-3-nitrophenylcarbonothioyl)hydrazine-1-carboxylate (1.16 g, 3.68 mmol) in EA (10 ml) was added HCl/EA (3M, 10 mL, 8.2 eq). The resulting mixture was stirred at room temperature for 2 hours. After the reaction was completed, the mixture was concentrated in vacuum to give 4-fluoro-3-nitrobenzothiohydrazide (600 mg, yield: 75%) as a yellow solid.

MS Calcd.: 215.0; MS Found: 214.1 [M−H]⁻.

### Step E: 5-(4-fluoro-3-nitrophenyl)-1,3,4-thiadiazol-2(3H)-one

A mixture of 4-fluoro-3-nitrobenzothiohydrazide (600 mg, 2.8 mmol), CDI (550 mg, 3.4 mmol) and TEA (84.8 mg, 8.4 mmol) in THE (6 mL) was stirred at room temperature for 1 hours. After the reaction was completed, the mixture was quenched with water (200 mL) and extracted with ethyl acetate (50 mL×3). The organic layer was combined and dried with Na₂SO₄. Then the mixture was filtered and concentrated in vacuum to give 5-(4-fluoro-3-nitrophenyl)-1,3,4-thiadiazol-2(3H)-one (700 mg crude) as a brown solid.

MS Calcd.: 241.0; MS Found: 240.1 [M−H]⁻. (need to be checked)

### Step F: (S)-5-(3-nitro-4-((oxetan-2-ylmethyl)amino)phenyl)-1,3,4-thiadiazol-2(3H)-one

To a solution of 5-(4-fluoro-3-nitrophenyl)-1,3,4-thiadiazol-2(3H)-one (700 mg, 2.90 mmol) and (S)-oxetan-2-ylmethanamine MsOH salt (690 mg, 3.78 mmol) in THE (10 mL) was added DIEA (1.12 g, 8.70 mmol) at room temperature. The reaction was stirred at 50° C. for 2 hours. After the reaction was completed, the reaction was quenched with water (50 mL) and extracted with ethyl acetate (100 mL×3). The organic layer was combined and washed with brine (100 mL×2), dried over sodium sulfate, filtered and concentrated in vacuum. The residue was purified by column chromatography to give (S)-5-(3-nitro-4-((oxetan-2-ylmethyl)amino)phenyl)-1,3,4-thiadiazol-2(3H)-one (280 mg, yield: 31%) as a red solid.

MS Calcd.: 308.06; MS Found: 309.0 [M+H]⁻.

### Step G: (S)-5-(3-amino-4-((oxetan-2-ylmethyl)amino)phenyl)-1,3,4-thiadiazol-2(3H)-one

To a solution of (S)-5-(3-nitro-4-((oxetan-2-ylmethyl)amino)phenyl)-1,3,4-thiadiazol-2(3H)-one (280 mg, 0.91 mmol) in MeOH (5 mL) was added Pd/C (wet, 10%, 100 mg). The mixture was stirred at room temperature under H₂ for 1 hours. After the reaction was finished, the reaction was filtered and concentrated to give (S)-5-(3-amino-4-((oxetan-2-ylmethyl)amino)phenyl)-1,3,4-thiadiazol-2(3H)-one (180 mg, yield: 71%) as a dark brown solid.

MS Calcd.: 278.1; MS Found: 279.0 [M+H]⁺.

### Step H: (S)-2-chloro-N-(2-((oxetan-2-ylmethyl)amino)-5-(5-oxo-4,5-dihydro-1,3,4-thiadiazol-2-yl)phenyl)acetamide

To a solution of (S)-5-(3-amino-4-((oxetan-2-ylmethyl)amino)phenyl)-1,3,4-thiadiazol-2(3H)-one (150 mg, 0.54 mmol) in THE (10 mL) was added 2-chloroacetic anhydride (110 mg, 0.64 mmol). The reaction was stirred at room temperature for 1 hour. Then the reaction was stirred at 60° C. for 48 hours. After the reaction was completed, the reaction was filtered and concentrated to give crude product which was purified by column chromatography to give (S)-2-chloro-N-(2-((oxetan-2-ylmethyl)amino)-5-(5-oxo-4,5-dihydro-1,3,4-thiadiazol-2-yl)phenyl)acetamide (90 mg, yield: 48%) as yellow oil.

MS Calcd.: 354.1; MS Found: 355.0 [M+H]⁺.

### Step I: 2-(4-(2-(4-chloro-2-fluorophenyl)-2-methylbenzo[d][1,3]dioxol-4-yl)piperidin-1-yl)-N-(2-((((S)-oxetan-2-yl)methyl)amino)-5-(5-oxo-4,5-dihydro-1,3,4-thiadiazol-2-yl)phenyl)acetamide

To a solution of (S)-2-chloro-N-(2-((oxetan-2-ylmethyl)amino)-5-(5-oxo-4,5-dihydro-1,3,4-thiadiazol-2-yl)phenyl)acetamide (150 mg, 0.45 mmol) and 4-(2-(4-chloro-2-fluorophenyl)-2-methylbenzo[d][1,3]dioxol-4-yl)piperidine (232 mg, 0.67 mmol) in DMF (2 mL) was added K₂CO₃ (186.3 mg, 1.35 mmol). The reaction was stirred at 70° C. for 16 hours. After the reaction was completed, the reaction was quenched with water (10 mL) and extracted with ethyl acetate (20 mL×3). The organic layer was combined and washed with brine (20 mL×2), dried over sodium sulfate, filtered and concentrated in vacuum to give crude product which was purified by column chromatography to give 2-(4-(2-(4-chloro-2-fluorophenyl)-2-methylbenzo[d][1,3]dioxol-4-yl)piperidin-1-yl)-N-(2-((((S)-oxetan-2-yl)methyl)amino)-5-(5-oxo-4,5-dihydro-1,3,4-thiadiazol-2-yl)phenyl)acetamide (90 mg, 30% yield) as yellow oil.

MS Calcd.: 665.2; MS Found: 666.0 [M+H]⁺.

### Step J: 5-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-2,3-dihydro-1,3,4-thiadiazol-2-one (Compound 221a)

To a solution of 2-(4-(2-(4-chloro-2-fluorophenyl)-2-methylbenzo[d][1,3]dioxol-4-yl)piperidin-1-yl)-N-(2-((((S)-oxetan-2-yl)methyl)amino)-5-(5-oxo-4,5-dihydro-1,3,4-thiadiazol-2-yl)phenyl)acetamide (90 mg, 0.14 mmol) in MeOH (3 mL) was added NaOH (2 M, 1 mL). The reaction was stirred at 70° C. for 8 hours. The solvent was removed in vacuo. The residue was purified by Prep-HPLC to give Compound 221a (10.99 mg, yield: 12%) as a white solid.

MS Calcd.: 647.2; MS Found: 648.3 [M+H]⁺.

¹H NMR (400 MHz, DMSO-d₆): 8.29 (s, 1H), 7.86 (s, 1H), 7.76 (d, J=8.8 Hz, 1H), 7.60 (dd, J=8.8 Hz, 1.6 Hz, 1H), 7.54-7.57 (m, 2H), 7.33 (dd, J=8.4 Hz, 2.0 Hz, 1H), 6.77-6.80 (m, 2H), 6.73-6.76 (m, 1H), 5.09-5.14 (m, 1H), 4.70-4.78 (m, 1H), 4.58-4.63 (m, 1H), 4.43-4.48 (m, 1H), 4.35-4.41 (m, 1H), 3.94 (d, J=12.8 Hz, 1H), 3.78 (d, J=14.0 Hz, 1H), 2.97-3.04 (m, 1H), 2.84-2.91 (m, 1H), 2.55-2.75 (m, 2H), 2.15-2.28 (m, 2H), 2.01-2.05 (m, 4H), 1.65-1.82 (m, 4H).

### Example 97: 3-[2-({4-[2-(4-chloro-2-fluorophenyl)-2-methyl-2H-1,3-benzodioxol-4-yl]piperidin-1-yl}methyl)-6-fluoro-1-{[(2S)-oxetan-2-yl]methyl}-1H-1,3-benzodiazol-5-yl]-4,5-dihydro-1,2,4-oxadiazol-5-one (Compound 222a)

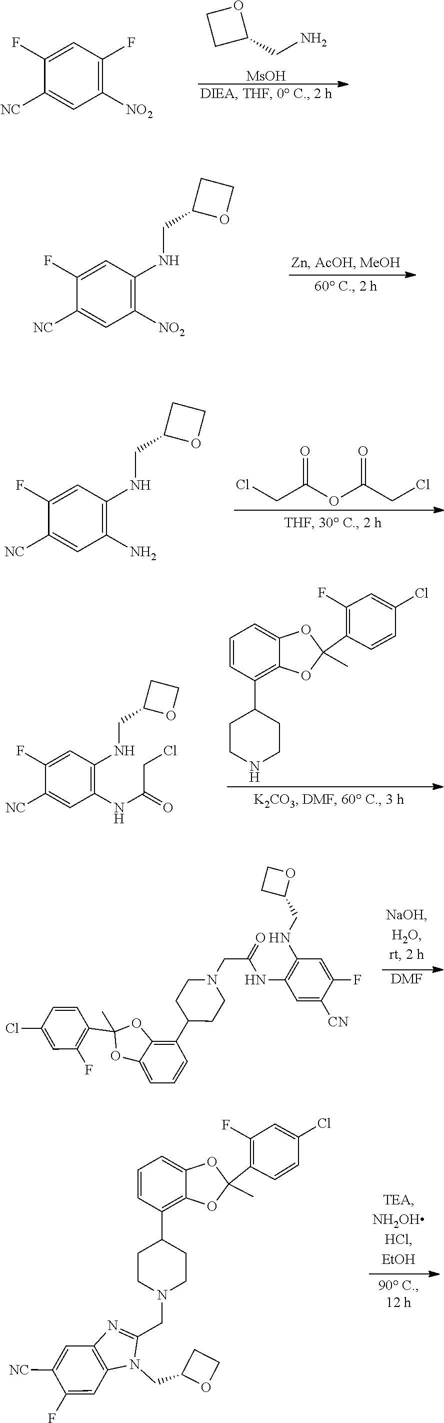

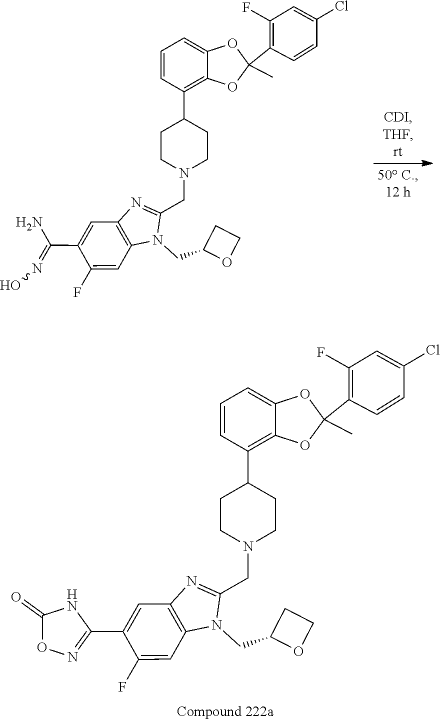 [SMILES: CC1(c2ccc(Cl)cc2F)Oc2cccc(C3CCN(Cc4nc5cc(C(N)=NO)c(F)cc5n4C[C@@H]4CCO4)CC3)c2O1 ; CC1(c2ccc(Cl)cc2F)Oc2cccc(C3CCN(Cc4nc5cc(-c6noc(=O)[nH]6)c(F)cc5n4C[C@@H]4CCO4)CC3)c2O1]

### Step A: (S)-2-fluoro-5-nitro-4-((oxetan-2-ylmethyl)amino)benzonitrile

To a solution 2,4-difluoro-5-nitrobenzonitrile (3 g, 6.3 mmol) in THE (30 mL) was added (S)-oxetan-2-ylmethanamine MsOH salt (2.69 g, 14.7 mmol) and DIEA (6 g, 49 mmol) successively at 0° C. The reaction mixture was stirred at 0° C. for 2 hours. After the reaction was completed, the reaction was quenched with H₂O and extracted with EA (30 mL*3). The organic layers were combined, dried over Na₂SO₄ and concentrated. The residue was purified by silica gel column chromatography (PE/EA=1/1) to give (S)-2-fluoro-5-nitro-4-((oxetan-2-ylmethyl)amino) benzonitrile (2.1 g, 70% yield) as a yellow solid.

MS Calcd.: 251.1; MS Found: 251.9 [M+H]⁺.

### Step B: (S)-5-amino-2-fluoro-4-((oxetan-2-ylmethyl)amino)benzonitrile

To a solution of (S)-2-fluoro-5-nitro-4-((oxetan-2-ylmethyl)amino)benzonitrile (2.1 g, 8.4 mmol) and Zn (5.5 g) in MeOH (21 mL) was added AcOH (1.51 g) at room temperature. The reaction mixture was stirred at 60° C. for 2 hours. After the reaction was completed, the reaction was filtered and the solid was washed with NaHCO₃ (aq). The filtrate was quenched with H₂O and extracted with EA (20 mL*3). The organic layers were combined, dried over Na₂SO₄ and concentrated to give (S)-5-amino-2-fluoro-4-((oxetan-2-ylmethyl)amino)benzonitrile (1.8 g, 85.7% yield) as a yellow solid.

MS Calcd.: 221.1; MS Found: 222.1 [M+H]⁺.

### Step C: (S)-2-chloro-N-(5-cyano-4-fluoro-2-((oxetan-2-ylmethyl)amino)phenyl) acetamide

To a solution of (S)-5-amino-2-fluoro-4-((oxetan-2-ylmethyl)amino) benzonitrile (1.8 g, 8.14 mmol) in THE (18 mL) was added 2-chloroacetic anhydride (1.7 g, 9.77 mmol). The reaction was stirred at 30° C. for 2 hours. After the reaction was completed, the reaction mixture was concentrated and the residue was purified by silica gel column chromatography (DCM/MeOH=10/1) to give (S)-2-chloro-N-(5-cyano-4-fluoro-2-((oxetan-2-ylmethyl)amino)phenyl) acetamide (610 mg, 30% yield) as yellow oil.

MS Calcd.: 297.1; MS Found: 298.1 [M+H]⁺.

### Step D: 2-(4-(2-(4-chloro-2-fluorophenyl)-2-methylbenzo[d][1,3]dioxol-4-yl)piperidin-1-yl)-N-(5-cyano-4-fluoro-2-((((S)-oxetan-2-yl)methyl)amino)phenyl) acetamide

To a mixture of 4-(2-(4-chloro-2-fluorophenyl)-2-methylbenzo[d][1,3]dioxol-4-yl)piperidine (583 mg, 1.68 mmol) and K₂CO₃ (697 mg, 5.05 mmol) in DMF (5 ml) was added (S)-2-chloro-N-(5-cyano-4-fluoro-2-((oxetan-2-ylmethyl)amino) phenyl)acetamide (500 mg, 1.68 mmol). The reaction was stirred at 60° C. for 3 hours. After the reaction was completed, the reaction was quenched with H₂O and extracted with EA (5 mL*3). The organic layers were combined, dried over Na₂SO₄ and concentrated to give 2-(4-(2-(4-chloro-2-fluorophenyl)-2-methylbenzo[d][1,3]dioxol-4-yl)piperidin-1-yl)-N-(5-cyano-4-fluoro-2-((((S)-oxetan-2-yl)methyl)amino)phenyl)acetamide (500 mg, yield: 85.7%) as a yellow solid which was used to the next step without purification.

MS Calcd.: 608.2; MS Found: 609.2 [M+H]⁺.

### Step E: 2-((4-(2-(4-chloro-2-fluorophenyl)-2-methylbenzo[d][1,3]dioxol-4-yl)piperidin-1-yl)methyl)-6-fluoro-1-(((S)-oxetan-2-yl)methyl)-1H-benzo[d]imidazole-5-carbonitrile

To a solution of 2-(4-(2-(4-chloro-2-fluorophenyl)-2-methylbenzo[d][1,3]dioxol-4-yl)piperidin-1-yl)-N-(5-cyano-4-fluoro-2-((((S)-oxetan-2-yl)methyl)amino)phenyl)acetamide (500 mg, 0.82 mmol) in DMF (5 mL) was added 2M NaOH (1 mL). The reaction was stirred at rt for 2 hours. After the reaction was completed, the reaction was quenched with H₂O and extracted with EA (5 mL*3). The organic layers were combined, dried over Na₂SO₄ and concentrated. The residue was purified by silica gel column chromatography (DCM/MeOH=10/1) to give 2-((4-(2-(4-chloro-2-fluorophenyl)-2-methylbenzo[d][1,3]dioxol-4-yl)piperidin-1-yl)methyl)-6-fluoro-1-(((S)-oxetan-2-yl)methyl)-1H-benzo[d]imidazole-5-carbonitrile (300 mg, yield: 60%) as colorless oil.

MS Calcd.: 590.2; MS Found: 591.2 [M+H]⁺.

### Step F: 2-((4-(2-(4-chloro-2-fluorophenyl)-2-methylbenzo[d][1,3]dioxol-4-yl)piperidin-1-yl)methyl)-6-fluoro-N′-hydroxy-1-(((S)-oxetan-2-yl)methyl)-1H-benzo[d]imidazole-5-carboximidamide

To a solution of 2-((4-(2-(4-chloro-2-fluorophenyl)-2-methylbenzo[d][1,3]dioxol-4-yl)piperidin-1-yl)methyl)-6-fluoro-1-(((S)-oxetan-2-yl)methyl)-1H-benzo[d]imidazole-5-carbonitrile (100 mg, 0.16 mmol) in EtOH (1 ml) was added NH₂OH·HCl (94.6 mg, 1.28 mmol) and TEA (137 mg, 1.28 mmol) successively. The reaction was stirred at 90° C. for 12 h. After the reaction was completed, the reaction was quenched with H₂O and extracted with EA (5 mL*3). The organic layers were combined, dried over Na₂SO₄ and concentrated. the reaction was purified by prep-HPLC to give 2-((4-(2-(4-chloro-2-fluorophenyl)-2-methylbenzo[d][1,3]dioxol-4-yl)piperidin-1-yl)methyl)-6-fluoro-N′-hydroxy-1-(((S)-oxetan-2-yl)methyl)-1H-benzo[d]imidazole-5-carboximidamide (80 mg, yield: 80%) as colorless oil.

MS Calcd.: 623.2; MS Found: 624.2[M+H]⁺.

### Step G: 3-[2-({4-[2-(4-chloro-2-fluorophenyl)-2-methyl-2H-1,3-benzodioxol-4-yl]piperidin-1-yl}methyl)-6-fluoro-1-{[(2S)-oxetan-2-yl]methyl}-1H-1,3-benzodiazol-5-yl]-4,5-dihydro-1,2,4-oxadiazol-5-one (Compound 222a)

To a solution of 2-((4-(2-(4-chloro-2-fluorophenyl)-2-methylbenzo[d][1,3]dioxol-4-yl)piperidin-1-yl)methyl)-6-fluoro-N′-hydroxy-1-(((S)-oxetan-2-yl)methyl)-1H-benzo[d]imidazole-5-carboximidamide (80 mg, 0.13 mmol) in THE (1 mL) was added CDI (31 mg, 0.19 mmol) and TEA (26 mg, 0.26 mmol) successively. The reaction was stirred at 50° C. for 12 h. The reaction mixture was purified by Prep-HPLC to give Compound 222a (1.07 mg, yield: 1%) as a white solid.

MS Calcd.: 649.2; MS Found: 650.2 [M+H]⁺.

¹H NMR (400 MHz, DMSO-d₆): δ 8.03 (d, J=5.6 Hz, 1H), 7.53-7.66 (m, 3H), 7.24-7.29 (m, 1H), 7.20 (d, J=8.8 Hz, 1H), 6.67-6.83 (m, 3H), 4.59-4.68 (m, 2H), 4.45-4.51 (m, 1H), 3.85-4.10 (m, 2H), 3.05-3.12 (m, 1H), 2.92-2.98 (m, 1H), 2.66-2.84 (m, 2H), 2.47-2.55 (m, 1H), 2.20-2.38 (m, 2H), 2.02-2.04 (m, 3H), 1.75-1.96 (m, 3H), 1.49-1.51 (m, 1H), 1.27-1.33 (m, 2H).

### Example 98: 2-({4-[2-(4-chloro-2-fluorophenyl)-2-methyl-2H-1,3-benzodioxol-4-yl]piperidin-1-yl}methyl)-6-fluoro-1-{[(2S)-oxetan-2-yl]methyl}-5-(1H-1,2,3,4-tetrazol-5-yl)-1H-1,3-benzodiazole (Compound 223a)

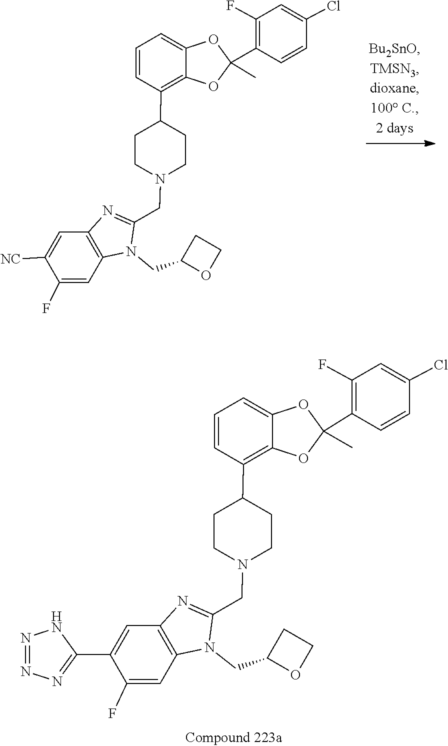 [SMILES: CC1(c2ccc(Cl)cc2F)Oc2cccc(C3CCN(Cc4nc5cc(C#N)c(F)cc5n4C[C@@H]4CCO4)CC3)c2O1 ; CC1(c2ccc(Cl)cc2F)Oc2cccc(C3CCN(Cc4nc5cc(-c6nnn[nH]6)c(F)cc5n4C[C@@H]4CCO4)CC3)c2O1]

To a mixture of 2-((4-(2-(4-chloro-2-fluorophenyl)-2-methylbenzo[d][1,3]dioxol-4-yl)piperidin-1-yl)methyl)-6-fluoro-1-(((S)-oxetan-2-yl)methyl)-1H-benzo[d]imidazole-5-carbonitrile (40 mg, 0.07 mmol) in dioxane (1 mL) was added TMSN₃ (40 mg, 0.35 mmol) and BuSnO₂ (52.3 mg, 0.21 mmol) successively. The reaction was stirred at 100° C. for 2 days. The reaction mixture was purified by Prep-HPLC to give Compound 223a (1.11 mg, yield: 2%) as a white solid.

MS Calcd.: 633.2; MS Found: 634.3 [M+H]⁺.

¹H NMR (400 MHz, DMSO-d₆): δ 8.40-8.44 (m, 1H), 7.44 (t, J=8.4 Hz, 1H), 7.03-7.13 (m, 3H), 6.63-6.78 (m, 3H), 5.05-5.15 (m, 1H), 4.58-4.62 (m, 1H), 4.25-4.39 (m, 2H), 4.12-4.22 (m, 3H), 3.50-3.63 (m, 2H), 2.78-2.89 (m, 2H), 2.60-2.74 (m, 4H), 2.35-2.42 (m, 1H), 2.05-2.20 (m, 1H), 1.92-2.04 (m, 4H).

### Example 99: 4-[2-(4-chloro-2-fluorophenyl)-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidine (Compound 224a)

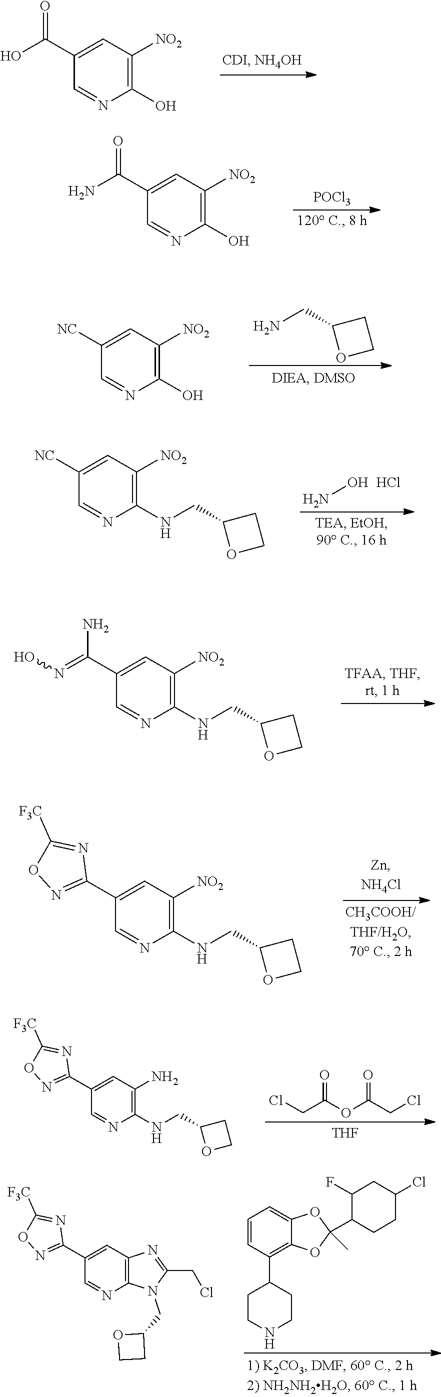

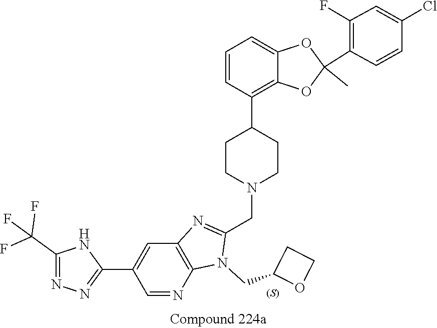

### Step A: 6-hydroxy-5-nitronicotinamide

To a solution of 6-hydroxy-5-nitronicotinic acid (5 g, 27.2 mmol) in DMF (25 mL) was added CDI (4.85 g, 29.9 mmol) at room temperature. The reaction was stirred at 50° C. for 2 hours. Then ammonia (35 mL, 25%-28% w/w aqueous solution) was added and the reaction was stirred at room temperature for 1 h. The reaction mixture was filtered and washed with water. The solid was dried to afford 6-hydroxy-5-nitronicotinamide (4.2 g, crude 86% yield) as a yellow solid.

### Step B: 6-chloro-5-nitronicotinonitrile

6-hydroxy-5-nitronicotinamide (4.2 g, 23.0 mmol) was dissolved in POCl₃ (40 mL). The reaction was stirred at 120° C. for 8 hours. The reaction was concentrated under reduced pressure to give crude product. The crude product was dissolved in ethyl acetate (100 mL) and washed with NaHCO₃ solution. The organic layer was washed with brine (50 mL×2), dried over sodium sulfate, filtered and concentrated in vacuum to give crude product. The crude product was purified by column chromatography (PE:EA=10:1) to afford 6-chloro-5-nitronicotinonitrile (2.7 g, 64% yield) as yellow solid.

### Step C: (S)-5-nitro-6-((oxetan-2-ylmethyl)amino)nicotinonitrile

To a solution of 6-chloro-5-nitronicotinonitrile (1 g, 5.5 mmol) and (S)-oxetan-2-ylmethanamine MsOH salt (1.2 g, 6.5 mmol) in DMSO (10 mL) was added DIEA (2.11 g, 16.5 mmol) at room temperature. The reaction was stirred at 70° C. for 1 hour. After the reaction was completed, the reaction was quenched with water (50 mL) and extracted with ethyl acetate (100 mL×3). The organic layer was combined and washed with brine (100 mL×2), dried over sodium sulfate, filtered and concentrated in vacuum. The residue was purified by column chromatography (PE:EA=3:1) to give (S)-3-nitro-4-((oxetan-2-ylmethyl)amino) benzonitrile (1.4 g, crude) as an orange solid.

MS Calcd.: 234.1; MS Found: 235.1 [M+H]⁺.

### Step D: (S)—N′-hydroxy-5-nitro-6-((oxetan-2-ylmethyl)amino)nicotinimidamide

To a solution of (S)-3-nitro-4-((oxetan-2-ylmethyl)amino) benzonitrile (1.4 g, 6.0 mmol) in EtOH (15 mL) was added Hydroxylamine hydrochloride (2.06 g, 30.0 mmol) and TEA (3.6 g, 36.0 mmol). The reaction was stirred at 90° C. for 16 hours. After the reaction was completed, the reaction was quenched with water (50 mL) and extracted with ethyl acetate (100 mL×3). The organic layers were combined and washed with brine (50 mL×2), dried over sodium sulfate, filtered and concentrated in vacuum. The residue was purified by column chromatography (DCM:MeOH=10:1) to give (S)—N′-hydroxy-5-nitro-6-((oxetan-2-ylmethyl)amino)nicotinimidamide (1.1 g, 69% yield) as yellow solid.

MS Calcd.: 267.1; MS Found: 268.1 [M+H]⁺.

### Step E: (S)-3-nitro-N-(oxetan-2-ylmethyl)-5-(5-(trifluoromethyl)-1,2,4-oxadiazol-3-yl)pyri dine-2-amine

To a solution of (S)—N′-hydroxy-5-nitro-6-((oxetan-2-ylmethyl)amino) nicotinimidamide (1.0 g, 3.7 mmol) in THE (10 mL) was added TFAA (3.14 g, 14.8 mmol) at room temperature. The reaction was stirred at room temperature for 1 hours. After the reaction was completed, the reaction was quenched with NaHCO₃ solution (50 mL) and extracted with ethyl acetate (100 mL×3). The organic layer was combined and washed with brine (50 mL×2), dried over sodium sulfate, filtered and concentrated in vacuum. The residue was purified by column chromatography (PE:EA=5:1) to give (S)-3-nitro-N-(oxetan-2-ylmethyl)-5-(5-(trifluoromethyl)-1,2,4-oxadiazol-3-yl)pyridine-2-amine (1.0 g, 76% yield) as yellow solid.

MS Calcd.: 345.1; MS Found: 346.1 [M+H]⁺.

### Step F: (S)—N2-(oxetan-2-ylmethyl)-5-(5-(trifluoromethyl)-1,2,4-oxadiazol-3-yl)pyridine-2,3-diamine

To a solution of (S)-3-nitro-N-(oxetan-2-ylmethyl)-5-(5-(trifluoromethyl)-1,2,4-oxadiazol-3-yl)pyridine-2-amine (950 mg, 2.75 mmol) in THF/H₂O (10 mL/1 mL) was added Zn (1.8 g, 27.5 mmol), NH₄Cl (2.9 g, 55.0 mmol) and CH₃COOH (0.5 ml). The reaction was stirred at 70° C. for 2 hours. After the reaction was completed, the reaction was filtered and concentrated in vacuum. The residue was purified by column chromatography (PE:EA=1:1) to give (S)—N2-(oxetan-2-ylmethyl)-5-(5-(trifluoromethyl)-1,2,4-oxadiazol-3-yl)pyridine-2,3-diamine (600 mg, 69% yield) as yellow solid.

MS Calcd.: 315.1; MS Found: 316.1 [M+H]⁺.

### Step G: (S)-3-(2-(chloromethyl)-3-(oxetan-2-ylmethyl)-3H-imidazo[4,5-b]pyridin-6-yl)-5-(trifluoromethyl)-1,2,4-oxadiazole

To a solution of (S)—N2-(oxetan-2-ylmethyl)-5-(5-(trifluoromethyl)-1,2,4-oxadiazol-3-yl)pyridine-2,3-diamine (600 mg, 1.9 mmol) in THE (6 mL) was added 2-chloroacetic anhydride (356 mg, 2.1 mmol) at room temperature. The reaction was stirred at room temperature for 1 hour. Then the reaction was stirred at 60° C. for 16 hours. After the reaction was completed, the reaction was concentrated in vacuum. The residue was purified by column chromatography (PE:EA=2:1) to give (S)-3-(2-(chloromethyl)-3-(oxetan-2-ylmethyl)-3H-imidazo[4,5-b]pyridin-6-yl)-5-(trifluoromethyl)-1,2,4-oxadiazole (440 mg, 62% yield) as yellow solid.

MS Calcd.: 373.1; MS Found: 374.0 [M+H]⁺.

### Step H: 4-[2-(4-chloro-2-fluorophenyl)-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidine (Compound 224a)

A mixture of (S)-3-(2-(chloromethyl)-3-(oxetan-2-ylmethyl)-3H-imidazo[4,5-b]pyridin-6-yl)-5-(trifluoro methyl)-1,2,4-oxadiazole (40 mg, 0.1 mmol), 4-(2-(4-chloro-2-fluorophenyl)-2-methylbenzo[d][1,3]dioxol-4-yl)piperidine (40 mg) and K₂CO₃ (44 mg, 0.3 mmol) in DMF (1 mL) was stirred at 60° C. for 2 hours. Then NH₂NH₂·H₂O (54 mg, 1.0 mmol) was added into the reaction. The reaction was stirred at 60° C. for 1 hour. After the reaction was completed, the reaction was filtered and purified by Prep-HPLC to give Compound 224a (15.81 mg, 22% yield) as a white solid.

MS Calcd.: 683.2; MS Found: 684.5 [M+H]⁺.

¹H NMR (400 MHz, DMSO-d₆): δ 9.00 (d, J=1.6 Hz, 1H), 8.58 (d, J=2.0 Hz, 1H), 7.53-7.59 (m, 2H), 7.33 (dd, J=8.4 Hz, J=1.6 Hz, 1H), 6.72-6.81 (m, 3H), 5.17-5.24 (m, 1H), 4.83-4.88 (m, 1H), 4.70-4.75 (m, 1H), 4.46-4.51 (m, 1H), 4.36-4.41 (m, 1H), 3.90-4.02 (m, 2H), 2.92-3.02 (m, 2H), 2.63-2.74 (m, 2H), 2.45-2.56 (m, 1H), 2.20-2.33 (m, 2H), 2.02 (s, 3H), 1.72-1.83 (m, 4H).

### Example 100: Compound 224b and Compound 224c

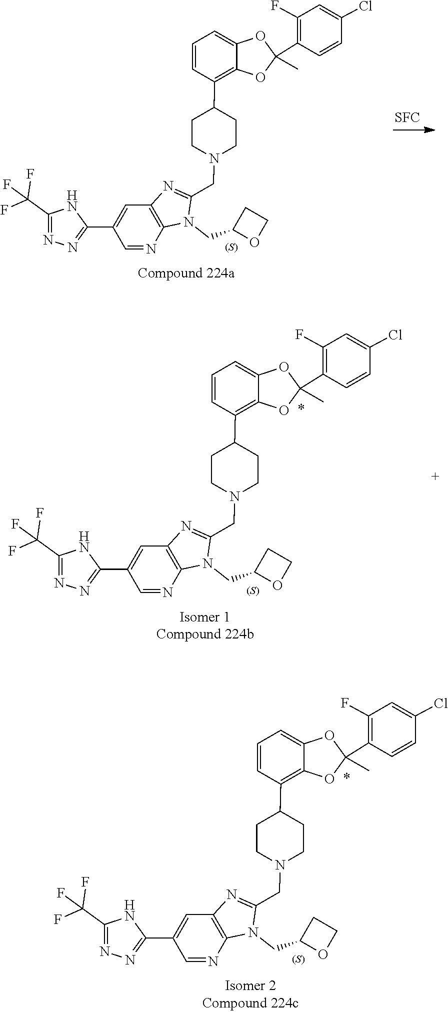

Compound 224a (300 mg, 0.44 mmol) was separated by SFC (Column: DAICELCHIRALCEL@OZ, 250*25 mm, 10 mm; Mobile phase A: Supercritical CO2, Mobile phase B: MeOH (+0.1% 7.0 mmol/L Ammonia in MeOH), A:B=70:30 at 80 ml/min; Column Temp: R^{T}; Back Pressure: 100 Bar; Wavelength: 214 nm.) to give Compound 224b (isomer 1, 107.37 mg, 36% yield) and Compound 224c (isomer 2, 125.6 mg, 42% yield) as a white solid.

Compound 224b:

Chiral analysis method: Column: DAICELCHIRALCEL@OZ, 100*3.0 mm, 3 mm; Mobile phase A: Supercritical CO2, Mobile phase B: MeOH (+0.1% DEA) Gradient: A:B=95:5 (0.00 min), 60:40 (5.50 min), 60:40 (8.00 min) at 1.5 mL/min; Column Temp: 35° C.; Back Pressure: 1800 psi; Wavelength: 214 nm. Rt=4.787 min.

MS Calcd.: 683.2; MS Found: 684.4 [M+H]⁺.

¹H NMR (400 MHz, DMSO-d₆): 15.38 (br s, 1H), 9.00 (d, J=2.0 Hz, 1H), 8.60 (d, J=1.6 Hz, 1H), 7.50-7.59 (m, 2H), 7.33 (dd, J=8.0 Hz, J=1.6 Hz, 1H), 6.70-6.80 (m, 3H), 5.18-5.24 (m, 1H), 4.86 (dd, J=14.8 Hz, J=6.4 Hz, 1H), 4.73 (dd, J=14.8 Hz, J=4.4 Hz, 1H), 4.43-4.51 (m, 1H), 4.34-4.41 (m, 1H), 3.90-4.02 (m, 2H), 2.98 (t, J=12.0 Hz, 2H), 2.62-2.75 (m, 2H), 2.45-2.55 (m, 1H), 2.21-2.33 (m, 2H), 2.02 (s, 3H), 1.72-1.83 (m, 4H).

Compound 224c:

Chiral analysis method: Column: DAICELCHIRALCEL@OZ, 100*3.0 mm, 3 mm; Mobile phase A: Supercritical CO2, Mobile phase B: MeOH (+0.1% DEA) Gradient: A:B=95:5 (0.00 min), 60:40 (5.50 min), 60:40 (8.00 min) at 1.5 mL/min; Column Temp: 35° C.; Back Pressure: 1800 psi; Wavelength: 214 nm. Rt=5.010 min.

MS Calcd.: 683.2; MS Found: 684.4 [M+H]⁺.

¹H NMR (400 MHz, DMSO-d₆): 15.39 (brs, 1H), 9.00 (d, J=2.0 Hz, 1H), 8.60 (d, J=1.6 Hz, 1H), 7.52-7.59 (m, 2H), 7.33 (dd, J=8.4 Hz, J=2.0 Hz, 1H), 6.72-6.82 (m, 3H), 5.17-5.24 (m, 1H), 4.86 (dd, J=14.8 Hz, J=6.8 Hz, 1H), 4.73 (dd, J=14.4 Hz, J=4.0 Hz, 1H), 4.45-4.51 (m, 1H), 4.35-4.41 (m, 1H), 4.00 (d, J=14.0 Hz, 1H), 3.94 (d, J=13.6 Hz, 1H), 2.94-3.04 (m, 2H), 2.63-2.74 (m, 2H), 2.46-2.55 (m, 1H), 2.23-2.35 (m, 2H), 2.02 (s, 3H), 1.70-1.86 (m, 4H).

### Example 101: 4-[2-(4-chlorophenyl)-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidine (Compound 225a)

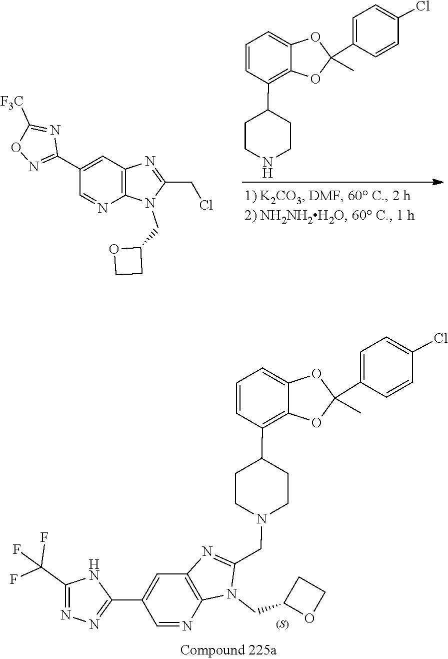

A mixture of (S)-3-(2-(chloromethyl)-3-(oxetan-2-ylmethyl)-3H-imidazo[4,5-b]pyridin-6-yl)-5-(trifluoro methyl)-1,2,4-oxadiazole (102 mg, 0.27 mmol), 4-(2-(4-chlorophenyl)-2-methylbenzo[d][1,3]dioxol-4-yl)piperidine (100 mg) and K₂CO₃ (113 mg, 0.81 mmol) in DMF (2 mL) was stirred at 60° C. for 2 hours. Then NH₂NH₂·H₂O (68 mg, 1.36 mmol) was added into the reaction. The reaction was stirred at 60° C. for 1 hour. After the reaction was completed, the reaction was filtered and purified by Prep-HPLC to give Compound 225a (55.24 mg, 30% yield) as a white solid.

MS Calcd.: 665.2; MS Found: 666.7 [M+H]⁺.

¹H NMR (400 MHz, DMSO-d₆): δ 9.00 (d, J=2.0 Hz, 1H), 8.59 (d, J=2.0 Hz, 1H), 7.59 (d, J=8.4 Hz, 2H), 7.49 (d, J=8.4 Hz, 2H), 6.70-6.80 (m, 3H), 5.17-5.25 (m, 1H), 4.83-4.88 (m, 1H), 4.69-4.76 (m, 1H), 4.43-4.51 (m, 1H), 4.36-4.42 (m, 1H), 3.90-4.02 (m, 2H), 2.92-3.05 (m, 2H), 2.62-2.74 (m, 2H), 2.47-2.54 (m, 1H), 2.21-2.32 (m, 2H), 1.97 (s, 3H), 1.70-1.83 (m, 4H).

### Example 102: 2-({4-[2-(4-chlorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 226a)

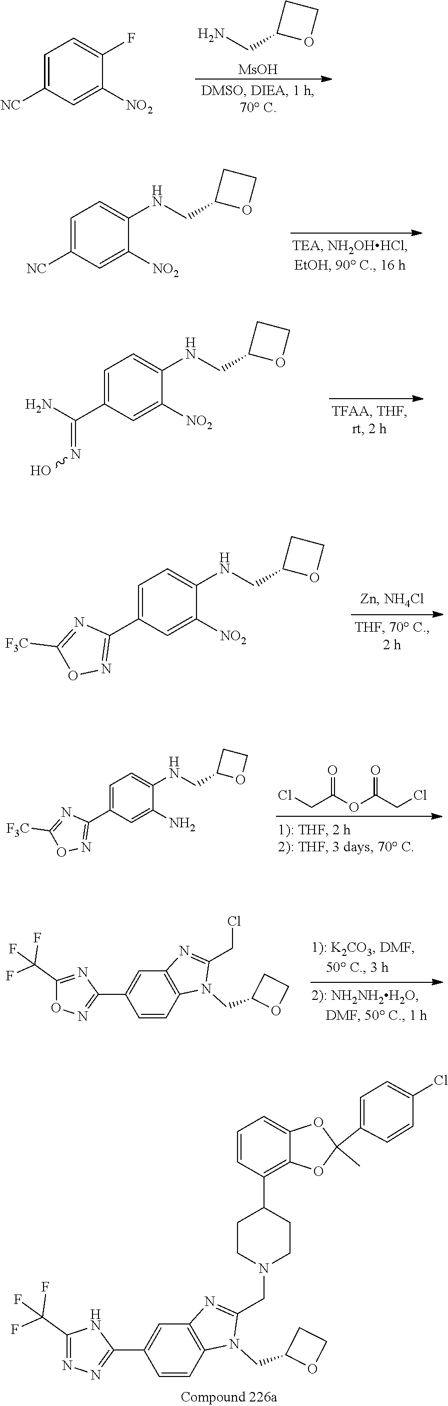

### Step A: (S)-3-nitro-4-((oxetan-2-ylmethyl)amino)benzonitrile

To a solution of 4-fluoro-3-nitrobenzonitrile (5 g, 30.1 mmol) and (S)-oxetan-2-ylmethanamine MsOH salt (6.06 g, 33.1 mmol) in DMSO (60 mL) was added DIEA (11.6 g, 90.3 mmol) at room temperature. The reaction was stirred at 70° C. for 1 hour. After the reaction was completed, the reaction was quenched with water (50 mL) and extracted with ethyl acetate (200 mL×3). The organic layer was combined and washed with brine (100 mL×2), dried over sodium sulfate, filtered and concentrated in vacuum. The residue was purified by column chromatography (PE:EA=3:1) to give (S)-3-nitro-4-((oxetan-2-ylmethyl)amino)benzonitrile (4.9 g, 70%) as an orange solid.

MS Calcd.: 233.1; MS Found: 234.1 [M+H]⁺.

### Step B: (S)—N′-hydroxy-3-nitro-4-((oxetan-2-ylmethyl)amino)benzimidamide

To a solution of (S)-3-nitro-4-((oxetan-2-ylmethyl)amino)benzonitrile (1 g, 4.3 mmol) in EtOH (10 mL) was added Hydroxylamine hydrochloride (1.8 g, 2.8 mmol) and TEA (1.73 g, 17.1 mmol). The reaction was stirred at 90° C. for 16 hours. After the reaction was completed, the reaction was quenched with water (50 mL) and extracted with ethyl acetate (100 mL×3). The organic layer was combined and washed with brine (50 mL×2), dried over sodium sulfate, filtered and concentrated in vacuum. The residue was purified by column chromatography (DCM:MeOH=10:1) to give (S)—N′-hydroxy-3-nitro-4-((oxetan-2-ylmethyl)amino)benzimidamide (840 mg, 73.4% yield) as red solid.

MS Calcd.: 266.1; MS Found: 267.1 [M+H]⁺.

### Step C: (S)-2-nitro-N-(oxetan-2-ylmethyl)-4-(5-(trifluoromethyl)-1,2,4-oxadiazol-3-yl)aniline

To a solution of (S)—N′-hydroxy-3-nitro-4-((oxetan-2-ylmethyl)amino)benzimidamide (300 mg, 1.13 mmol) in THE (5 mL) was added TFAA (945 g, 4.5 mmol) at room temperature. The reaction was stirred at room temperature for 2 hours. After the reaction was completed, the reaction was quenched with NaHCO₃ solution (50 mL) and extracted with ethyl acetate (50 mL×3). The organic layer was combined and washed with brine (50 mL×2), dried over sodium sulfate, filtered and concentrated in vacuum. The residue was purified by column chromatography (PE:EA=5:1) to give (S)-2-nitro-N-(oxetan-2-ylmethyl)-4-(5-(trifluoromethyl)-1,2,4-oxadiazol-3-yl)aniline (300 mg, 77.3% yield) as yellow solid.

MS Calcd.: 344.1; MS Found: 345.1 [M+H]⁺.

### Step D: (S)—N1-(oxetan-2-ylmethyl)-4-(5-(trifluoromethyl)-1,2,4-oxadiazol-3-yl)benzene-1,2-diamine

To a solution of (S)-2-nitro-N-(oxetan-2-ylmethyl)-4-(5-(trifluoromethyl)-1,2,4-oxadiazol-3-yl)aniline (300 mg, 0.87 mmol) in THF/H₂O (10 mL/1 ml) was added Zn (1.8 g, 27.5 mmol) and NH₄Cl (2.9 g, 55.0 mmol) at room temperature. The reaction was stirred at 70° C. for 2 hours. After the reaction was completed, the reaction was filtered and concentrated in vacuum. The residue was purified by column chromatography (PE:EA=1:1) to give (S)—N1-(oxetan-2-ylmethyl)-4-(5-(trifluoromethyl)-1,2,4-oxadiazol-3-yl)benzene-1,2-diamine (230 mg, 85% yield) as yellow solid. MS Calcd.: 314.1; MS Found: 315.1 [M+H]⁺.

### Step E: (S)-3-(2-(chloromethyl)-1-(oxetan-2-ylmethyl)-1H-benzo[d]imidazol-5-yl)-5-(trifluoromethyl)-1,2,4-oxadiazole

To a solution of (S)—N1-(oxetan-2-ylmethyl)-4-(5-(trifluoromethyl)-1,2,4-oxadiazol-3-yl)benzene-1,2-diamine (230 mg, 0.73 mmol) in THE (6 mL) was added 2-chloroacetic anhydride (137 mg, 0.80 mmol) at room temperature. The reaction was stirred at room temperature for 2 hours. Then the reaction was stirred at 70° C. for 3 days. After the reaction was completed, the reaction was concentrated in vacuum. The residue was purified by column chromatography (PE:EA=2:1) to give (S)-3-(2-(chloromethyl)-1-(oxetan-2-ylmethyl)-1H-benzo[d]imidazol-5-yl)-5-(trifluoromethyl)-1,2,4-oxadiazole (120 mg, 34% yield) as yellow solid.

MS Calcd.: 372.1; MS Found: 373.1 [M+H]⁺.

### Step F: 2-({4-[2-(4-chlorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 226a)

A mixture of (S)-3-(2-(chloromethyl)-1-(oxetan-2-ylmethyl)-1H-benzo[d]imidazol-5-yl)-5-(trifluoromethyl)-1,2,4-oxadiazole (40 mg, 0.1 mmol), 4-(2-(4-chlorophenyl)-2-methylbenzo[d][1,3]dioxol-4-yl)-1l2-piperidine (60 mg, 0.16 mmol) and K₂CO₃ (44 mg, 0.3 mmol) in DMF (1 mL) was stirred at 50° C. for 3 hours. Then NH₂NH₂·H₂O (54 mg, 1.0 mmol) was added into the reaction. The reaction was stirred at 50° C. for 1 hour. After the reaction was completed, the reaction was filtered and the filtrate was purified by Prep-HPLC to give Compound 226a (10 mg, 16% yield) as a white solid.

MS Calcd.: 664.2; MS Found: 665.7 [M+H]⁺.

¹H NMR (400 MHz, DMSO-d₆): δ 8.12 (s, 1H), 7.91 (dd, J=8.8 Hz, 1.2 Hz, 1H), 7.48-7.61 (m, 5H), 6.72-6.78 (m, 3H), 5.11-5.18 (m, 1H), 4.67-4.72 (m, 1H), 4.55-4.60 (m, 1H), 4.39-4.50 (m, 2H), 3.91 (d, J=13.2 Hz, 1H), 3.74 (dd, J=14.0 Hz, 2.0 Hz, 1H), 3.01-3.04 (m, 1H), 2.89-2.92 (m, 1H), 2.60-2.75 (m, 2H), 2.33-2.45 (m, 1H), 2.10-2.29 (m, 2H), 1.97 (s, 3H), 1.68-1.82 (m, 4H).

### Example 103: 2-({4-[2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]-1-{[(2S)-oxetan-2-yl]methyl}-1H-1,3-benzodiazole (Compound 227a)

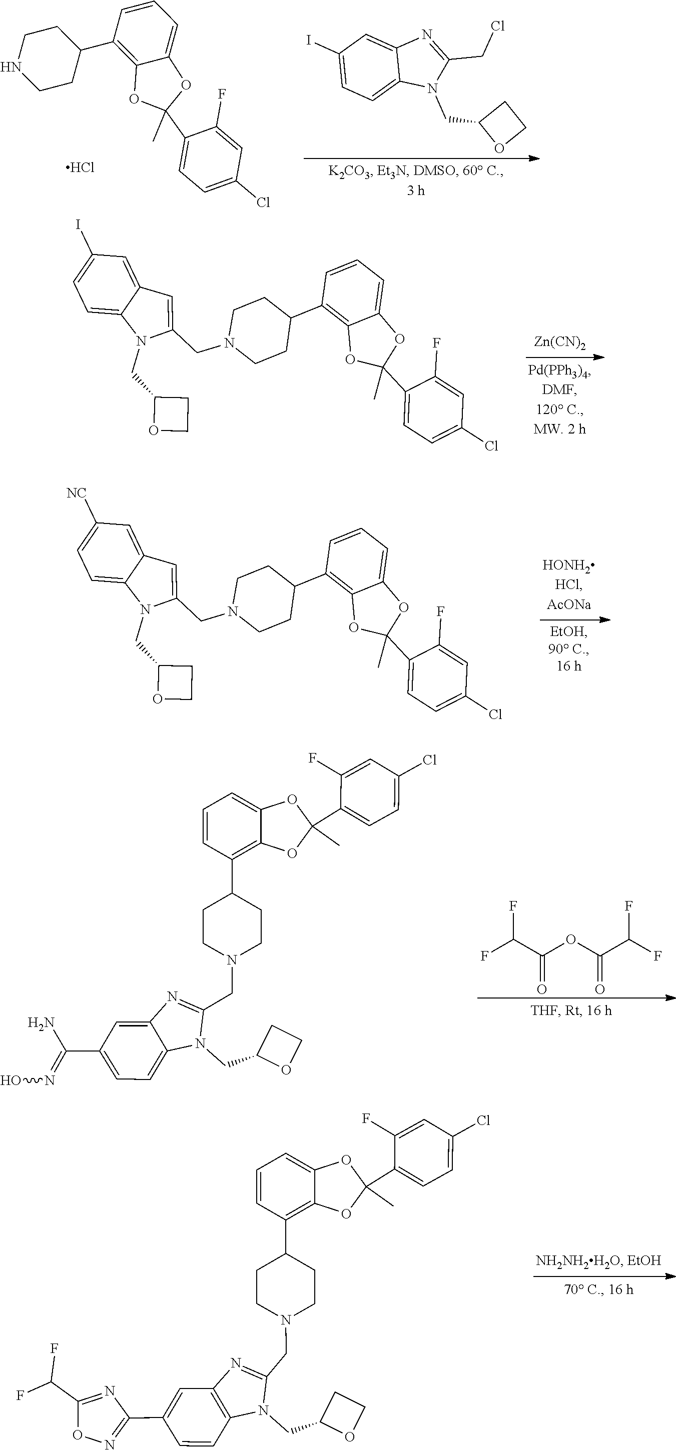

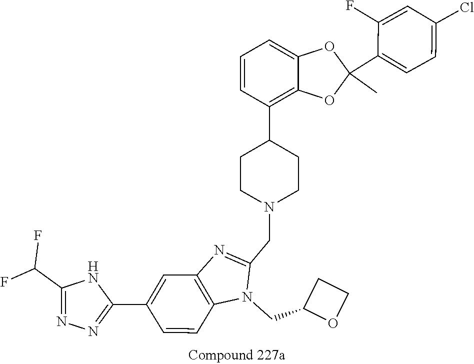 [SMILES: CC1(c2ccc(Cl)cc2F)Oc2cccc(C3CCN(Cc4nc5cc(-c6nnc(C(F)F)[nH]6)ccc5n4C[C@@H]4CCO4)CC3)c2O1]

### Step A 2-((4-(2-(4-chloro-2-fluorophenyl)-2-methylbenzo[d][1,3]dioxol-4-yl)piperidin-1-yl)methyl)-5-iodo-1-(((S)-oxetan-2-yl)methyl)-1H-benzo[d]imidazole

A mixture of 4-(2-(4-chloro-2-fluorophenyl)-2-methylbenzo[d][1,3]dioxol-4-yl)piperidine (600 mg, 1.56 mmol) in DMSO (8 mL) was added Et₃N (473.6 mg, 4.68 mmol) and K₂CO₃ (645.8 mg, 6.48 mmol), stirred at room temperature for 10 mins. Then (S)-2-(chloromethyl)-5-iodo-1-(oxetan-2-ylmethyl)-1H-benzo[d]imidazole (530 mg, 1.56 mmol) was added and the mixture was stirred at 60° C. for 3 hours. The mixture was poured into cold water (40 mL) and extracted with EtOAc (30 mL*2), the combined organic layer was washed with brine, dried over sodium sulfate, filtered and the residue was purified by column chromatography to furnish 2-((4-(2-(4-chloro-2-fluorophenyl)-2-methylbenzo[d][1,3]dioxol-4-yl)piperidin-1-yl)methyl)-5-iodo-1-(((S)-oxetan-2-yl)methyl)-1H-benzo[d]imidazole (650 mg, yield: 61.9%) as brown oil.

MS Calcd.: 673.1; MS Found: 674.0 [M+H]⁺.

### Step B: 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonitrile

A mixture of 2-((4-(2-(4-chloro-2-fluorophenyl)-2-methylbenzo[d][1,3]dioxol-4-yl)piperidin-1-yl)methyl)-5-iodo-1-(((S)-oxetan-2-yl)methyl)-1H-benzo[d]imidazole (80 mg, 0.12 mmol), Zn(CN)₂ (42 mg, 0.36 mmol), and Pd(PPh₃)₄ (14 mg, 0.01 mmol) in DMF (2 mL) was stirred at 120° C. under Ar with microwave for 2 hours. The mixture was filtered and the residue was purified by silica gel column chromatography to furnish 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonitrile (50 mg, yield: 73.5%) as yellow oil.

MS Calcd.: 572.2; MS Found: 573.1 [M+H]⁺.

### Step C: 2-((4-(2-(4-chloro-2-fluorophenyl)-2-methylbenzo[d][1,3]dioxol-4-yl)piperidin-1-yl)methyl)-N′-hydroxy-1-(((S)-oxetan-2-yl)methyl)-1H-benzo[d]imidazole-5-carboximidamide

A mixture of 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonitrile (50 mg, 0.0874 mmol), NH₂OH·HCl (61 mg, 0.874 mmol) and NaOAC (86 mg, 1.0488 mmol) in EtOH (5 mL) was stirred at 90° C. for 16 hours. The reaction mixture was poured into cold water (30 mL) and extracted with EtOAc (30 mL*2), the combined organic layer was washed with brine, dried over sodium sulfate, filtered and concentrated under reduced pressure to furnish 2-((4-(2-(4-chloro-2-fluorophenyl)-2-methylbenzo[d][1,3]dioxol-4-yl)piperidin-1-yl)methyl)-N′-hydroxy-1-(((S)-oxetan-2-yl)methyl)-1H-benzo[d]imidazole-5-carboximidamide (53 mg,) as yellow solid.

MS Calcd.: 605.2; MS Found: 606.1 [M+H]⁺.

### Step D: 3-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-5-(difluoromethyl)-1,2,4-oxadiazole

To a solution of 2-((4-(2-(4-chloro-2-fluorophenyl)-2-methylbenzo[d][1,3]dioxol-4-yl)piperidin-1-yl)methyl)-N′-hydroxy-1-(((S)-oxetan-2-yl)methyl)-1H-benzo[d]imidazole-5-carboximidamide (53 mg) in THF (3 mL) was added 2,2-difluoroacetic anhydride (76 mg, 0.437 mmol) in THE (1 mL) dropwisely at 0° C. The mixture was stirred at room temperature for 16 hours. The reaction mixture was poured into cold water (30 mL) and extracted with EtOAc (30 mL*2), the combined organic layer was washed with brine, dried over sodium sulfate, filtered and concentrated under reduced pressure. The residue was purified by silica gel column chromatography to furnish 3-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-5-(difluoromethyl)-1,2,4-oxadiazole (32 mg, yield: 54.9%) as white solid.

MS Calcd.: 665.2; MS Found: 666.2 [M+H]⁺.

### Step E: 2-({4-[2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]-1-{[(2S)-oxetan-2-yl]methyl}-1H-1,3-benzodiazole (Compound 227a)

A mixture of 3-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-11H-benzo[d]imidazol-5-yl)-5-(difluoromethyl)-1,2,4-oxadiazole (32 mg, 0.048 mmol), and NH₂NH₂·H₂O (0.3 mL) in EtOH (2 mL) was stirred at 70° C. for 16 hours. The reaction mixture was purified by prep-HPLC to give Compound 227a (8.2 mg, yield: 25.6%) as white solid.

MS Calcd.: 664.2; MS Found: 665.2 [M+H]⁺.

¹H NMR (400 MHz, MeOH-d₄) 8.28 (s, 1H), 7.96 (d, J=8.8 Hz, 1H), 7.82 (d, J=10.0 Hz, 1H), 7.58 (t, J=8.0 Hz, 1H), 7.28 (d, J=10.8 Hz, 1H), 7.20 (d, J=9.2 Hz, 1H), 6.90-6.65 (m, 4H), 5.35-5.22 (m, 1H), 4.78-4.68 (m, 2H), 4.66-4.59 (m, 1H), 4.51-4.45 (m, 1H), 4.05-4.01 (m, 1H), 3.92-3.85 (m, 1H), 3.14-3.06 (m, 1H), 2.97-2.90 (m, 1H), 2.88-2.78 (m, 1H), 2.75-2.63 (m, 1H), 2.60-2.50 (m, 1H), 2.38-2.23 (m, 2H), 2.03 (s, 3H), 1.96-1.75 (m, 4H).

¹⁹FNMR −112.35, −112.44, −118.25.

### Example 104: 2-{4-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-1H-1,2,3-triazol-1-yl}ethan-1-ol (Compound 228a)

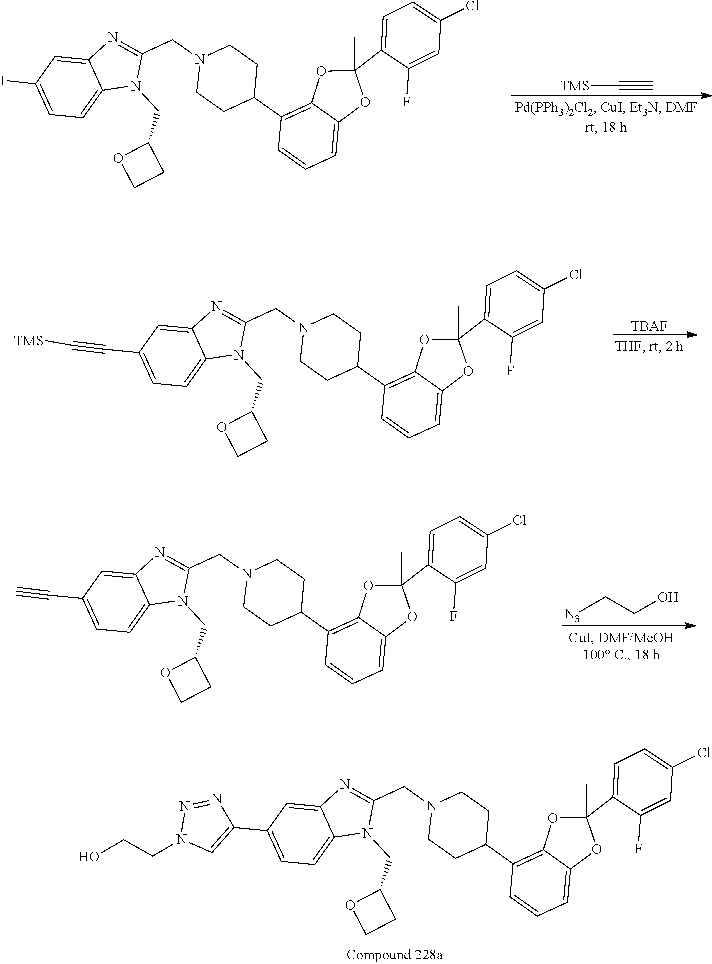

### Step A: 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-((trimethylsilyl)ethynyl)-1H-benzo[d]imidazole

A mixture of ethynyltrimethylsilane (76 mg, 0.780 mmol), Pd(PPh₃)₂Cl₂ (26 mg, 0.023 mmol), 2-((4-(2-(4-chloro-2-fluorophenyl)-2-methylbenzo[d][1,3]dioxol-4-yl)piperidin-1-yl)methyl)-5-iodo-1-(((S)-oxetan-2-yl)methyl)-1H-benzo[d]imidazole (150 mg, 0.223 mmol) and CuI (4.4 mg, 0.023 mmol) in Et₃N/DMF (v:v=1:1, 8.0 mL) was stirred at room temperature for 18 h under Ar atmosphere. The reaction mixture was diluted with brine (50 mL) and extracted with EA (15 mL×3). The combined organic layers were washed with brine (10 mL), dried over anhydrous Na₂SO₄ and concentrated under vacuum. The residue was purified by Prep-TLC to furnish 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-((trimethylsilyl)ethynyl)-1H-benzo[d]imidazole (80 mg, 55.8%) as a yellow oil.

MS Calcd.: 643.2; MS Found: 644.1 [M+H]⁺.

### Step B: 2-((4-(2-(4-chloro-2-fluorophenyl)-2-methylbenzo[d][1,3]dioxol-4-yl)piperidin-1-yl)methyl)-5-ethynyl-1-(((S)-oxetan-2-yl)methyl)-1H-benzo[d]imidazole

A mixture of TBAF (0.25 mL, 0.248 mmol, 1 N in THF) and 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-((trimethylsilyl)ethynyl)-1H-benzo[d]imidazole (80 mg, 0.124 mmol) in THE (3.0 mL) was stirred at room temperature for 2 h. The reaction mixture was concentrated under vacuum to furnish 2-((4-(2-(4-chloro-2-fluorophenyl)-2-methylbenzo[d][1,3]dioxol-4-yl)piperidin-1-yl)methyl)-5-ethynyl-1-(((S)-oxetan-2-yl)methyl)-1H-benzo[d]imidazole (85 mg) as crude brown oil.

MS Calcd.: 571.2; MS Found: 572.1 [M+H]⁺.

### Step C: 2-{4-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-1H-1,2,3-triazol-1-yl}ethan-1-ol (Compound 228a)

A mixture of 2-azidoethanol (39 mg, 0.444 mmol), CuI (2.8 mg, 0.015 mmol), MeOH (1.0 mL), 2-((4-(2-(4-chloro-2-fluorophenyl)-2-methylbenzo[d][1,3]dioxol-4-yl)piperidin-1-yl)methyl)-5-ethynyl-1-(((S)-oxetan-2-yl)methyl)-1H-benzo[d]imidazole (85 mg) in DMF (4.0 ml) was stirred at 100° C. for 18 h under Ar atmosphere. Upon cooling down, the solids were filtrated out. The filtrate was directly purified by Prep-HPLC to furnish Compound 228a (14.2 mg, 15.0%) as a white solid.

MS Calcd.: 658.2; MS Found: 659.1 [M+H]⁺.

¹H NMR (400 MHz, MeOD) δ 8.27 (s, 1H), 8.03 (s, 1H), 7.74 (d, J=8.0 Hz, 1H), 7.62 (d, J=8.0 Hz, 1H), 7.49 (t, J=8.0 Hz, 1H), 7.19 (dt, J=11.2, 2.0 Hz, 1H), 7.11 (d, J=8.0 Hz, 1H), 6.75-6.57 (m, 3H), 5.18 (s, 1H), 4.72-4.69 (m, 1H), 4.61-4.50 (m, 2H), 4.46 (t, J=5.2 Hz, 2H), 4.42-4.30 (m, 1H), 4.17-3.95 (m, 2H), 3.90 (t, J=5.2 Hz, 2H), 2.75-2.62 (m, 2H), 2.57-2.38 (m, 3H), 1.93-1.73 (m, 9H).

### Example 105: 2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 229a); (isomer 1:Compound 229b; isomer 2:Compound 229c)

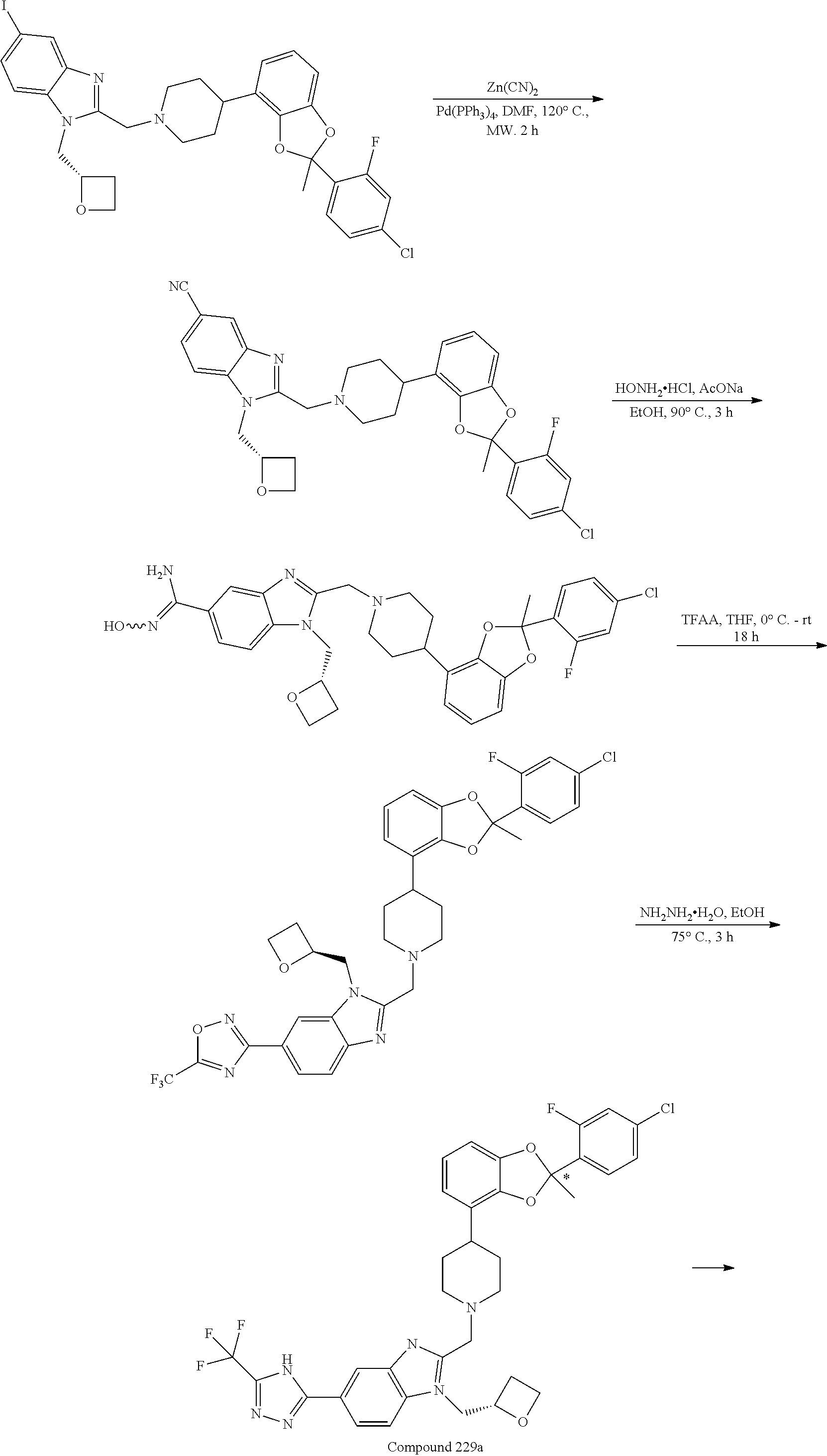

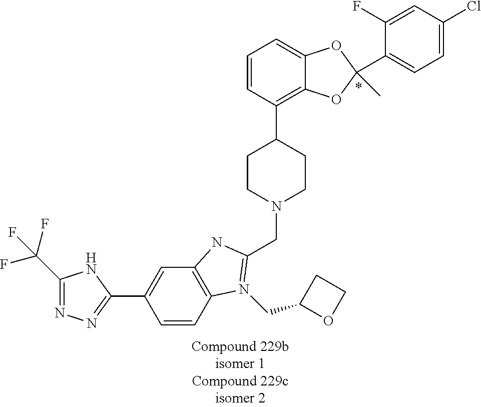

### Step A: 2-((4-(2-(4-chloro-2-fluorophenyl)-2-methylbenzo[d][1,3]dioxol-4-yl)piperidin-1-yl)methyl)-1-((S)-oxetan-2-ylmethyl)-1H-benzo[d]imidazole-5-carbonitrile

A mixture of Zn(CN)₂ (330 mg, 2.808 mmol), Pd(PPh₃)₄ (107 mg, 0.094 mmol) and 2-((4-(2-(4-chloro-2-fluorophenyl)-2-methylbenzo[d][1,3]dioxol-4-yl)piperidin-1-yl)methyl)-5-iodo-1-((S)-oxetan-2-ylmethyl)-1H-benzo[d]imidazole (630 mg, 0.936 mmol) in DMF (10 ml) was stirred at 120° C. for 2 hours under Ar atmosphere with microwave irradiation. Upon cooling down, the reaction mixture was directly purified by RFC to furnish 2-((4-(2-(4-chloro-2-fluorophenyl)-2-methylbenzo[d][1,3]dioxol-4-yl)piperidin-1-yl)methyl)-1-((S)-oxetan-2-ylmethyl)-1H-benzo[d]imidazole-5-carbonitrile (330 mg, yield: 61.6%) as a white solid.

MS Calcd.: 572.2; MS Found: 573.1 [M+H]⁺.

### Step B: 2-((4-(2-(4-chloro-2-fluorophenyl)-2-methylbenzo[d][1,3]dioxol-4-yl)piperidin-1-yl)methyl)-N′-hydroxy-1-((S)-oxetan-2-ylmethyl)-1H-benzo[d]imidazole-5-carboximidamide

A mixture of AcONa (412 mg, 5.028 mmol), NH₂OH·HCl (291 mg, 4.194 mmol) and 2-((4-(2-(4-chloro-2-fluorophenyl)-2-methylbenzo[d][1,3]dioxol-4-yl)piperidin-1-yl)methyl)-1-((S)-oxetan-2-ylmethyl)-1H-benzo[d]imidazole-5-carbonitrile (240 mg, 0.419 mmol) in EtOH (4.0 ml) was at 90° C. for 3 h in a sealed tube. Upon cooling down, the mixture was diluted with H₂O (20 mL) and extracted with EtOAc (3×10 mL). The combined organic layers were dried over anhydrous Na₂SO₄ and concentrated under vacuum to furnish 2-((4-(2-(4-chloro-2-fluorophenyl)-2-methylbenzo[d][1,3]dioxol-4-yl)piperidin-1-yl)methyl)-N′-hydroxy-1-((S)-oxetan-2-ylmethyl)-1H-benzo[d]imidazole-5-carboximidamide (210 mg, yield: 70.9%) as a white solid.

MS Calcd.: 605.2; MS Found: 606.1 [M+H]⁺.

### Step C: 3-(2-((4-(2-(4-chloro-2-fluorophenyl)-2-methylbenzo[d][1,3]dioxol-4-yl)piperidin-1-yl)methyl)-1-((S)-oxetan-2-ylmethyl)-1H-benzo[d]imidazol-6-yl)-5-(trifluoromethyl)-1,2,4-oxadiazole

To a stirred solution of 2-((4-(2-(4-chloro-2-fluorophenyl)-2-methylbenzo[d][1,3]dioxol-4-yl)piperidin-1-yl)methyl)-N′-hydroxy-1-((S)-oxetan-2-ylmethyl)-1H-benzo[d]imidazole-5-carboximidamide (145 mg) in dry THE (10 mL) was added a solution of TFAA (252 mg, 1.201 mmol) in THE (2.0 ml) dropwisely at 0° C. The final reaction mixture was stirred at room temperature for 18 hours. The reaction mixture was diluted with saturated NaHCO₃ (30 mL) and extracted with EA (3×10 mL). The combined organic layers were dried over anhydrous Na₂SO₄ and concentrated under vacuum to furnish 3-(2-((4-(2-(4-chloro-2-fluorophenyl)-2-methylbenzo[d][1,3]dioxol-4-yl)piperidin-1-yl)methyl)-1-((S)-oxetan-2-ylmethyl)-1H-benzo[d]imidazol-6-yl)-5-(trifluoromethyl)-1,2,4-oxadiazole (90 mg, crude) as crude yellow solid.

MS Calcd.: 683.2; MS Found: 684.1 [M+H]⁺.

### Step D: 2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 229a)

To a mixture of 3-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-5-(trifluoromethyl)-1,2,4-oxadiazole (35.0 mg, 0.0512 mmol) in ethanol (4 mL) was added hydrazine hydrate (98% in H₂O) (26.0 mg, 0.512 mmol). The resulting mixture was stirred at 75° C. for 3 hours. The reaction mixture was diluted with H₂O (5 mL) and extracted with mixed solvent (DCM:CH₃OH=10:1) (10 mL*3). The combined organic phase was dried over Na₂SO₄, and filtered. The filtrate was concentrated and purified by prep-HPLC (0.1% NH₄HCO₃ in water and acetonitrile) to afford 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-(5-(trifluoromethyl)-1H-1,2,4-triazol-3-yl)-1H-benzo[d]imidazole as a white solid (25.8 mg, 74% yield).

¹H NMR (400 MHz, DMSO-d₆) δ 15.20 (br.s, 1H), 8.28 (s, 1H), 7.93 (dd, J=8.8, 1.2 Hz, 1H), 7.84 (d, J=8.4 Hz, 1H), 7.50-7.63 (m, 2H), 7.34 (dd, J=8.4, 2.0 Hz, 1H), 6.78-6.81 (m, 2H), 6.73-6.77 (m, 1H), 5.06-5.20 (m, 1H), 4.73-4.83 (m, 1H), 4.60-4.70 (m, 1H), 4.36-4.53 (m, 2H), 3.72-4.04 (m, 2H), 2.85-3.10 (m, 2H), 2.60-2.79 (m, 2H), 2.39-2.48 (m, 1H), 2.12-2.34 (m, 2H), 2.03 (s, 3H), 1.94-2.00 (m, 1H), 1.70-1.82 (m, 3H).

MS Calcd.: 682.2; MS Found: 683.2 [M+H]⁺.

### Step E: 2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 229b isomer 1; Compound 229c, isomer 2)

2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole was separated by SFC (Column: DAICELCHIRALPAK@IA, 250*25 mm, 10 mm; System: SHIMADZU LC-20AP; Mobile Phase A: n-Hexane, Mobile Phase B: EtOH (+0.1% 7.0 mmol/L Ammonia in MeOH), A:B=80:20; Flow: 30 mL/min; Injection: 5 mL; Cycle time: 25 min) to furnish Compound 229b and Compound 229c.

Compound 229b (8.06 mg, yield: 9.7%): white solid.

SFC condition: Column: DAICELCHIRALPAK@IA, 250*4.6 mm, 5 mm; System: SHIMADZU LC-20AP; Mobile Phase A: n-Hexane, Mobile Phase B: EtOH (+0.2% DEA), A:B=60:40; Flow: 1 mL/min; Injection: 1 mL; Cycle time: 30 min. Rt=5.985 min.

MS Calcd.: 682.2; MS Found: 683.0 [M+H]⁺.

¹H NMR (400 MHz, MeOD) δ 8.31 (d, J=0.8 Hz, 1H), 8.00 (dd, J=8.4, 1.2 Hz, 1H), 7.72 (d, J=8.8 Hz, 1H), 7.58 (t, J=8.4 Hz, 1H), 7.28 (dd, J=10.8, 2.0 Hz, 1H), 7.20 (dd, J=11.2, 1.6 Hz, 1H), 6.79-6.68 (m, 3H), 5.33-5.27 (m, 1H), 4.89-4.83 (m, 1H), 4.71 (dd, J=15.2, 2.4 Hz, 1H), 4.65-4.59 (m, 1H), 4.51-4.45 (m, 1H), 4.01 (d, J=13.6 Hz, 1H), 3.86 (d, J=13.6 Hz, 1H), 3.10-3.03 (m, 1H), 2.96-2.91 (m, 1H), 2.84-2.77 (m, 1H), 2.72-2.65 (m, 1H), 2.59-2.50 (m, 1H), 2.35-2.20 (m, 2H), 2.03 (s, 3H), 1.98-1.85 (m, 2H), 1.83-1.76 (m, 2H).

¹⁹F-NMR (400 MHz, MeOD): δ: −65.35, −112.42

Compound 229c (9.46 mg, yield: 11.5%): white solid.

SFC condition: Column: DAICELCHIRALPAK@IA, 250*4.6 mm, 5 mm; System: SHIMADZU LC-20AP; Mobile Phase A: n-Hexane, Mobile Phase B: EtOH (+0.2% DEA), A:B=60:40; Flow: 1 mL/min; Injection: 1 mL; Cycle time: 30 min. Rt=6.632 min.

MS Calcd.: 682.2; MS Found: 683.0 [M+H]⁺.

¹H NMR (400 MHz, MeOD) δ 8.30 (d, J=0.8 Hz, 1H), 8.00 (dd, J=8.8, 1.2 Hz, 1H), 7.75 (d, J=8.8 Hz, 1H), 7.58 (t, J=8.4 Hz, 1H), 7.28 (dd, J=10.8, 2.0 Hz, 1H), 7.21 (dd, J=11.2, 2.0 Hz, 1H), 6.79-6.68 (m, 3H), 5.32-5.25 (m, 1H), 4.89-4.83 (m, 1H), 4.72 (dd, J=15.2, 2.4 Hz, 1H), 4.66-4.60 (m, 1H), 4.50-4.45 (m, 1H), 4.00 (d, J=13.6 Hz, 1H), 3.90 (d, J=13.6 Hz, 1H), 3.09-3.03 (m, 1H), 2.97-2.90 (m, 1H), 2.84-2.77 (m, 1H), 2.74-2.66 (m, 1H), 2.59-2.51 (m, 2H), 2.36-2.20 (m, 2H), 2.03 (s, 3H), 1.95-1.75 (m, 4H).

¹⁹F-NMR (400 MHz, MeOD): δ: −65.81, −112.35

### Example 106: 3-(2-((4-(2-(4-chloro-2-fluorophenyl)-2-methylbenzo[d][1,3]dioxol-4-yl)piperidin-1-yl)methyl)-1-(2-methoxyethyl)-1H-benzo[d]imidazol-5-yl)-1,2,4-oxadiazol-5(4H)-one (Compound 230)

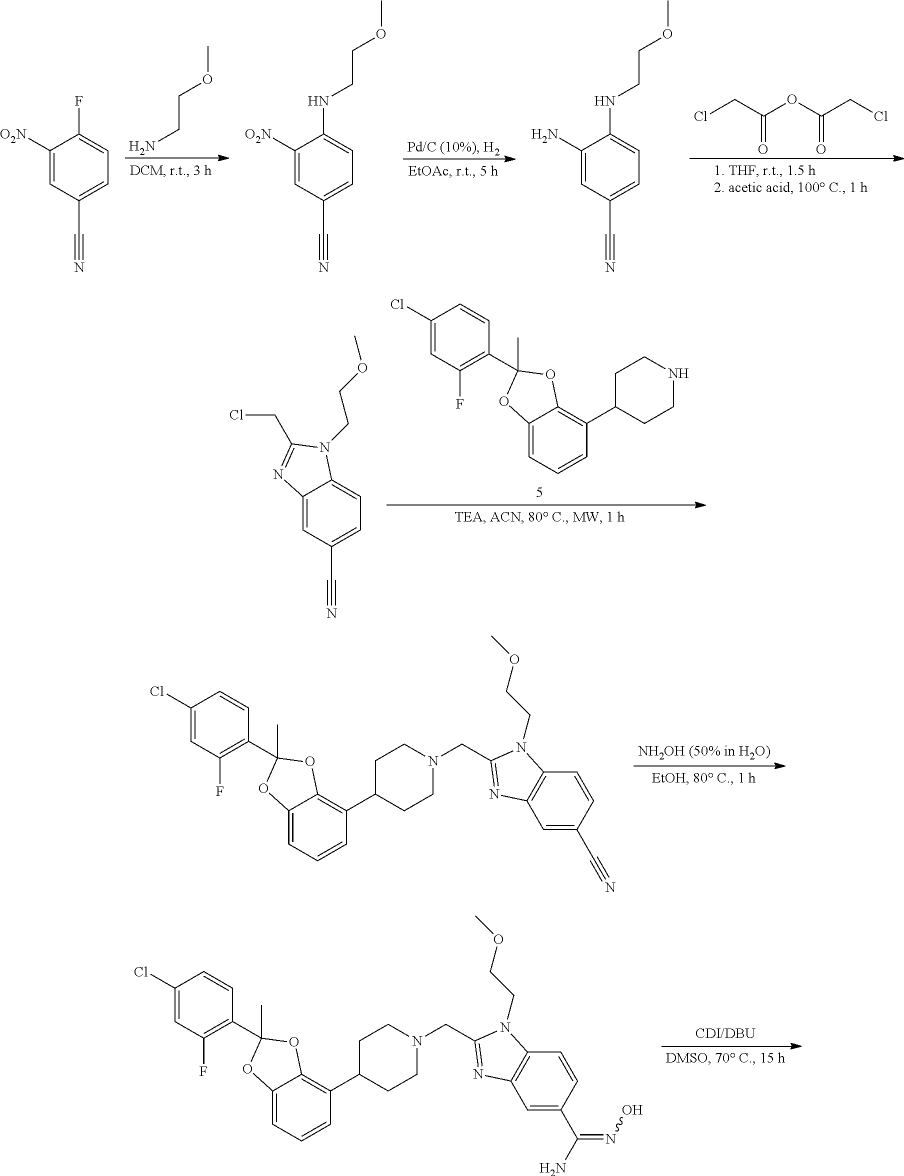

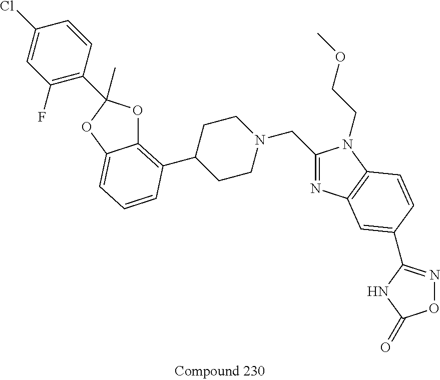 [SMILES: COCCn1c(CN2CCC(c3cccc4c3OC(C)(c3ccc(Cl)cc3F)O4)CC2)nc2cc(-c3noc(=O)[nH]3)ccc21]

### Step A: 4-((2-methoxyethyl)amino)-3-nitrobenzonitrile

To a solution of 4-fluoro-3-nitrobenzonitrile (700 mg, 4.22 mmol) in DCM (21 mL) was added 2-methoxyethan-1-amine (0.74 mL, 8.43 mmol). The mixture was stirred at room temperature for 3 h. The reaction mixture was concentrated and purified by flash chromatography (eluted with PE/EtOAc=3/1) to afford 4-((2-methoxyethyl)amino)-3-nitrobenzonitrile as a yellow solid (920 mg, 99% yield).

MS Calcd.: 221.1; MS Found: 222.2 [M+H]⁺.

### Step B: 3-amino-4-((2-methoxyethyl)amino)benzonitrile

To a solution of 4-((2-methoxyethyl)amino)-3-nitrobenzonitrile (880 mg, 3.98 mmol) in EtOAc (20 mL) was added Pd/C (10% w/w) (212 mg, 0.199 mmol). The mixture was stirred at room temperature for 5 h under H₂. The reaction mixture was filtered and the filtrate was concentrated to afford 3-amino-4-((2-methoxyethyl)amino)benzonitrile as a yellow solid (720 mg, 95% yield, crude).

MS Calcd.: 191.1; MS Found: 192.2 [M+H]⁺.

### Step C: 2-(chloromethyl)-1-(2-methoxyethyl)-1H-benzo[d]imidazole-5-carbonitrile

A solution of 3-amino-4-((2-methoxyethyl)amino)benzonitrile (650 mg) in THE (17 mL) was added 2-chloroacetic anhydride (760 mg, 4.42 mmol). The mixture was stirred at room temperature for 1.5 h. The reaction mixture was concentrated. The residue was dissolved in acetic acid (17 mL) and stirred at 100° C. for 1 h. The reaction mixture was concentrated and adjusted the pH to 8 with NaHCO₃ (aq.). Then the mixture was diluted with water (15 mL) and extracted with DCM (3×30 mL). The combined organic phase was washed with brine (30 mL), dried over anhydrous Na₂SO₄ and concentrated. The residue was purified by flash chromatography (eluted with DCM/MeOH=15/1) to afford 2-(chloromethyl)-1-(2-methoxyethyl)-1H-benzo[d]imidazole-5-carbonitrile as a white solid (850 mg, 100% yield).

MS Calcd.: 249.1; MS Found: 250.2 [M+H]⁺.

### Step D: 2-((4-(2-(4-chloro-2-fluorophenyl)-2-methylbenzo[d][1,3]dioxol-4-yl)piperidin-1-yl)methyl)-1-(2-methoxyethyl)-1H-benzo[d]imidazole-5-carbonitrile

To a solution of 2-(chloromethyl)-1-(2-methoxyethyl)-11H-benzo[d]imidazole-5-carbonitrile (72.0 mg, 0.287 mmol) and 4-(2-(4-chloro-2-fluorophenyl)-2-methylbenzo[d][1,3]dioxol-4-yl)piperidine (110 mg, 0.287 mmol) in ACN (2 mL) was added TEA (0.4 mL, 2.87 mmol). The mixture was stirred at 80° C. for 1 h under microwave irradiation. Then the mixture was diluted with water (5 mL) and extracted with DCM (3×10 mL). The combined organic phase was washed with brine (10 mL), dried over anhydrous Na₂SO₄ and concentrated. The residue was purified by flash chromatography (eluted with DCM/MeOH=15/1) to afford 2-((4-(2-(4-chloro-2-fluorophenyl)-2-methylbenzo[d][1,3]dioxol-4-yl)piperidin-1-yl)methyl)-1-(2-methoxyethyl)-1H-benzo[d]imidazole-5-carbonitrile a light yellow solid (170 mg, 79% yield).

MS Calcd.: 560.2; MS Found: 561.2 [M+H]⁺.

### Step E: 2-((4-(2-(4-chloro-2-fluorophenyl)-2-methylbenzo[d][1,3]dioxol-4-yl)piperidin-1-yl)methyl)-N′-hydroxy-1-(2-methoxyethyl)-1H-benzo[d]imidazole-5-carboximidamide

To a solution of 2-((4-(2-(4-chloro-2-fluorophenyl)-2-methylbenzo[d][1,3]dioxol-4-yl)piperidin-1-yl)methyl)-1-(2-methoxyethyl)-1H-benzo[d]imidazole-5-carbonitrile (130 mg, 0.232 mmol) in EtOH (2 mL) was added NH₂OH (50% in water) (153 mg, 2.32 mmol). The mixture was stirred at 80° C. for 1 h. Then the mixture was concentrated and purified by flash chromatography (eluted with DCM/MeOH=15/1) to afford 2-((4-(2-(4-chloro-2-fluorophenyl)-2-methylbenzo[d][1,3]dioxol-4-yl)piperidin-1-yl)methyl)-N′-hydroxy-1-(2-methoxyethyl)-1H-benzo[d]imidazole-5-carboximidamide as a light yellow solid (130 mg, 94% yield).

MS Calcd.: 593.2; MS Found: 594.2 [M+H]⁺.

### Step F: 3-(2-((4-(2-(4-chloro-2-fluorophenyl)-2-methylbenzo[d][1,3]dioxol-4-yl)piperidin-1-yl)methyl)-1-(2-methoxyethyl)-1H-benzo[d]imidazol-5-yl)-1,2,4-oxadiazol-5(4H)-one

To a solution of 2-((4-(2-(4-chloro-2-fluorophenyl)-2-methylbenzo[d][1,3]dioxol-4-yl)piperidin-1-yl)methyl)-N′-hydroxy-1-(2-methoxyethyl)-1H-benzo[d]imidazole-5-carboximidamide (120 mg, 0.202 mmol) in DMSO (2 mL) were added CDI (164 mg, 1.01 mmol) and DBU (310 mg, 2.02 mmol).

The mixture was stirred at 70° C. for 15 h. Then the mixture was diluted with water (5 mL) and extracted with EtOAc (3×10 mL). The combined organic phase was washed with brine (10 mL), dried over anhydrous Na₂SO₄ and concentrated. The residue was purified by prep-HPLC (0.1% formic acid in water and acetonitrile) to afford 3-(2-((4-(2-(4-chloro-2-fluorophenyl)-2-methylbenzo[d][1,3]dioxol-4-yl)piperidin-1-yl)methyl)-1-(2-methoxyethyl)-1H-benzo[d]imidazol-5-yl)-1,2,4-oxadiazol-5(4H)-one (Compound 230) as a white solid (49.6 mg, 40% yield).

¹H NMR (400 MHz, DMSO-d₆) δ 12.87 (br.s, 1H), 8.05 (d, J=1.6 Hz, 1H), 7.76 (d, J=8.6 Hz, 1H), 7.70 (dd, J=8.4, 1.6 Hz, 1H), 7.50-7.59 (m, 2H), 7.33 (dd, J=8.4, 2.0 Hz, 1H), 6.68-6.81 (m, 3H), 4.58 (t, J=5.2 Hz, 2H), 3.80-3.90 (m, 2H), 3.77 (t, J=5.6 Hz, 2H), 3.22 (s, 3H), 2.90-3.00 (m, 2H), 2.60-2.70 (m, 1H), 2.16-2.29 (m, 2H), 2.01 (s, 3H), 1.66-1.82 (m, 4H).

MS Calcd.: 619.2; MS Found: 620.2 [M+H]⁺.

### Example 107: 1-((2-((4-(2-(4-chloro-2-fluorophenyl)-2-methylbenzo[d][1,3]dioxol-4-yl)piperidin-1-yl)methyl)-5-(5-oxo-4,5-dihydro-1,2,4-oxadiazol-3-yl)-1H-benzo[d]imidazol-1-yl)methyl)cyclopropane-1-carbonitrile (Compound 231)

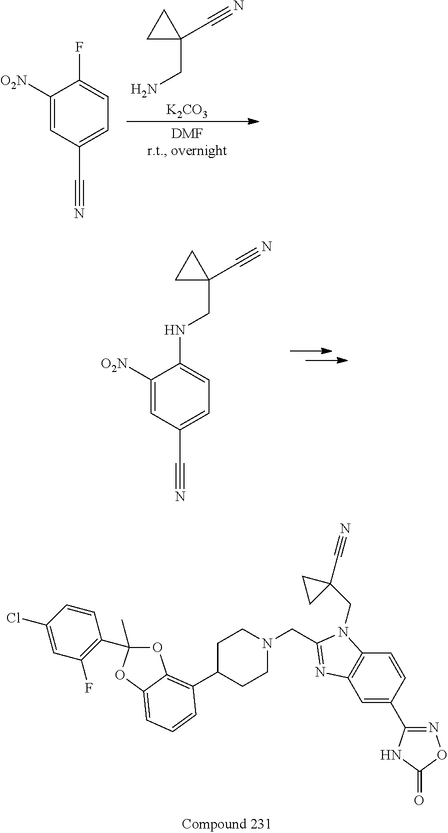

### Step A: 4-(((1-cyanocyclopropyl)methyl)amino)-3-nitrobenzonitrile

To a solution of 4-fluoro-3-nitrobenzonitrile (280 mg, 1.69 mmol) and 1-(aminomethyl)cyclopropane-1-carbonitrile (224 mg, 1.69 mmol) in DMF (8 mL) was added K₂CO₃ (467 mg, 3.38 mmol). The mixture was stirred at room temperature overnight. Then the mixture was diluted with water (15 mL) and extracted with EtOAc (3×30 mL). The combined organic phase was washed with brine (30 mL), dried over anhydrous Na₂SO₄ and concentrated. The residue was purified by flash chromatography (eluted with PE/EtOAc=4/1˜ 3/1) to afford 4-(((1-cyanocyclopropyl)methyl)amino)-3-nitrobenzonitrile as yellow solid (410 mg, 90% yield).

MS Calcd.: 242.1; MS Found: 243.2 [M+H]⁺.

### Step B: 1-((2-((4-(2-(4-chloro-2-fluorophenyl)-2-methylbenzo[d][1,3]dioxol-4-yl)piperidin-1-yl)methyl)-5-(5-oxo-4,5-dihydro-1,2,4-oxadiazol-3-yl)-1H-benzo[d]imidazol-1-yl)methyl)cyclopropane-1-carbonitrile (Compound 231)

The synthesis of Compound 231 is similar to that of Compound 230 except 4-(((1-cyanocyclopropyl)methyl)amino)-3-nitrobenzonitrile is used instead of 3-amino-4-((2-methoxyethyl)amino)benzonitrile in step B.

¹H NMR (400 MHz, DMSO-d₆) δ 8.08 (s, 1H), 7.89 (d, J=8.4 Hz, 1H), 7.75 (d, J=8.4 Hz, 1H), 7.51-7.60 (m, 2H), 7.33 (dd, J=8.4, 2.0 Hz, 1H), 6.70-6.82 (m, 3H), 4.73 (s, 2H), 3.93 (s, 2H), 2.97 (d, J=10.8 Hz, 2H), 2.61-2.73 (m, 1H), 2.22 (t, J=10.4 Hz, 2H), 2.02 (s, 3H), 1.64-1.81 (m, 4H), 1.30-1.48 (m, 4H).

MS Calcd.: 640.2; MS Found: 641.2 [M+H]⁺.

### Example 108: 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-(1H-tetrazol-5-yl)-1H-benzo[d]imidazole (Compound 232a)

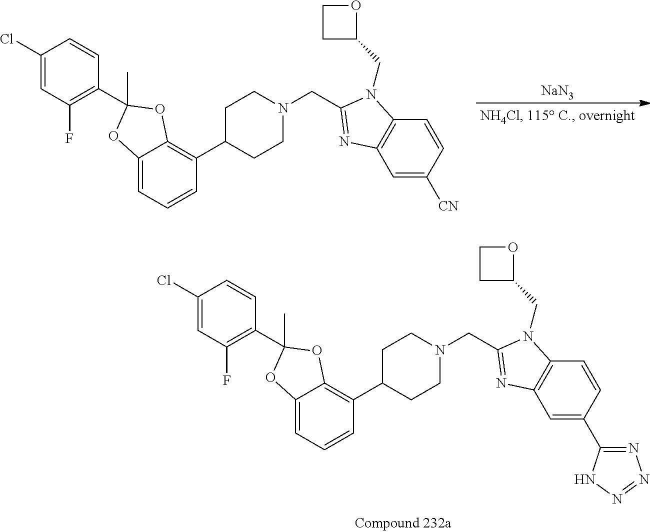 [SMILES: CC1(c2ccc(Cl)cc2F)Oc2cccc(C3CCN(Cc4nc5cc(C#N)ccc5n4C[C@@H]4CCO4)CC3)c2O1 ; CC1(c2ccc(Cl)cc2F)Oc2cccc(C3CCN(Cc4nc5cc(-c6nnn[nH]6)ccc5n4C[C@@H]4CCO4)CC3)c2O1 ; "NaN3"]

To a mixture of 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonitrile (40.0 mg, 0.0700 mmol) in DMF (1.0 mL) were added NH₄Cl (7.50 mg, 0.140 mmol), NaN₃ (9.10 mg, 0.140 mmol) at room temperature. The mixture in sealing tube was stirred at 115° C. overnight. The mixture was diluted with H₂O (3 mL), and extracted with EtOAc (3 mL*3). The organic layer was dried over Na₂SO₄, and concentrated. The residue was purified with Prep-HPLC (0.1% formic acid in H₂O and MeOH) to afford 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-(1H-tetrazol-5-yl)-1H-benzo[d]imidazole as a white solid (7.00 mg, 16% yield).

¹H NMR (400 MHz, DMSO-d₆) δ 8.10-8.25 (m, 2H), 7.92 (d, J=8.4 Hz, 1H), 7.68 (d, J=8.8 Hz, 1H), 7.50-7.60 (m, 2H), 7.34 (dd, J=8.4, 2.0 Hz, 1H), 6.68-6.82 (m, 3H), 5.08-5.20 (m, 1H), 4.73 (dd, J=15.2, 7.2 Hz, 1H), 4.57-4.64 (m, 1H), 4.44-4.52 (m, 1H), 4.38-4.44 (m, 1H), 3.93 (d, J=13.6 Hz, 1H), 3.78 (d, J=13.6 Hz, 1H), 3.03 (d, J=10.8 Hz, 1H), 2.90 (d, J=10.8 Hz, 1H), 2.62-2.74 (m, 2H), 2.41-2.48 (m, 1H), 2.15-2.33 (m, 2H), 2.03 (s, 3H), 1.66-1.82 (m, 4H).

MS Calcd.: 615.2; MS Found: 616.2 [M+H]⁺.

### Example 109: 2-((4-(2-(4-chloro-2-fluorophenyl)-2-methylbenzo[d][1,3]dioxol-4-yl)piperidin-1-yl)methyl)-5-(1-methyl-1H-pyrazol-4-yl)-1-(((S)-oxetan-2-yl)methyl)-1H-benzo[d]imidazole (Compound 233a)

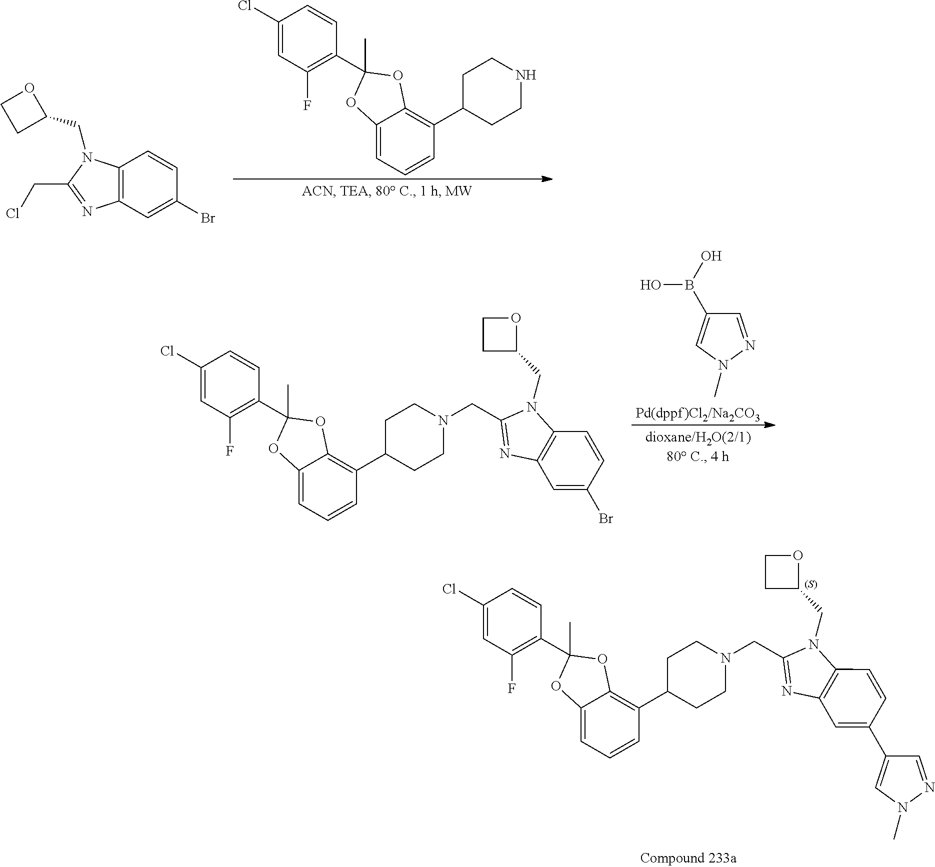

### Step A: 5-bromo-2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole

To a mixture of (S)-5-bromo-2-(chloromethyl)-1-(oxetan-2-ylmethyl)-1H-benzo[d]imidazole (1.70 g, 5.38 mmol) in ACN (8 mL) were added 4-(2-(4-chloro-2-fluorophenyl)-2-methylbenzo[d][1,3]dioxol-4-yl)piperidine (1.87 g, 5.38 mmol), and TEA (5.40 g, 53.8 mmol)) at room temperature. The mixture was stirred at 80° C. for 1 hour under microwave irradiation. The mixture was filtered, and the filter cake was washed with EtOAc (20 mL). The mixture was concentrated, and purified with silica gel column (PE/EtOAc=3/1 to 2/1) to afford 5-bromo-2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole as a yellow to white solid (1.75 g, 51% yield).

MS Calcd.: 625.1; MS Found: 626.2 [M+H]⁺.

### Step B: 2-((4-(2-(4-chloro-2-fluorophenyl)-2-methylbenzo[d][1,3]dioxol-4-yl)piperidin-1-yl)methyl)-5-(1-methyl-1H-pyrazol-4-yl)-1-(((S)-oxetan-2-yl)methyl)-1H-benzo[d]imidazole (Compound 233a)

To a solution of 5-bromo-2-((4-(2-(4-chloro-2-fluorophenyl)-2-methylbenzo[d][1,3] dioxol-4-yl)piperidin-1-yl)methyl)-1-(((S)-oxetan-2-yl)methyl)-1H-benzo[d]imidazole (120 mg, 0.192 mmol) and (1-methyl-1H-pyrazol-4-yl)boronic acid (48.4 mg, 0.384 mmol) in dioxane/H₂O (0.8 mL/0.4 mL) were added Pd(dppf)Cl₂ (7.00 mg, 0.0100 mmol) and Na₂CO₃ (61.1 mg, 0.576 mmol). The mixture was degassed and refilled with N₂ for three times and stirred at 80° C. for 4 h under microwave irradiation. After concentrated in vacuo, the residue was purified by prep-HPLC (0.1% formic acid in water and acetonitrile) to give 2-((4-(2-(4-chloro-2-fluorophenyl)-2-methylbenzo[d][1,3]dioxol-4-yl)piperidin-1-yl)methyl)-5-(1-methyl-1H-pyrazol-4-yl)-1-(((S)-oxetan-2-yl)methyl)-1H-benzo[d]imidazole as a white solid (45.0 mg, 37% yield).

¹H NMR (400 MHz, DMSO-d₆) δ 8.10 (s, 1H), 7.84 (s, 1H), 7.74 (d, J=1.6 Hz, 1H), 7.50-7.62 (m, 3H), 7.42 (dd, J=8.4, 1.6 Hz, 1H), 7.34 (dd, J=8.4, 2.0 Hz, 1H), 6.71-6.82 (m, 3H), 5.05-5.18 (m, 1H), 4.69 (dd, J=15.2, 6.8 Hz, 1H), 4.56 (d, J=15.2 Hz, 1H), 4.42-4.48 (m, 1H), 4.34-4.42 (m, 1H), 3.88-3.91 (m, 1H), 3.86 (s, 3H), 3.75 (d, J=13.2 Hz, 1H), 3.01 (d, J=11.2 Hz, 1H), 2.88 (d, J=11.2 Hz, 1H), 2.63-2.72 (m, 2H), 2.35-2.49 (m, 1H), 2.12-2.27 (m, 2H), 2.02 (s, 3H), 1.68-1.81 (m, 4H).

MS Calcd.: 627.2; MS Found: 628.2 [M+H]⁺.

### Example 110: 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-(1H-pyrazol-4-yl)-1H-benzo[d]imidazole (Compound 234a)

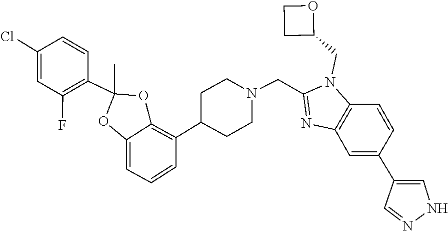 [SMILES: CC1(c2ccc(Cl)cc2F)Oc2cccc(C3CCN(Cc4nc5cc(-c6cn[nH]c6)ccc5n4C[C@@H]4CCO4)CC3)c2O1]

Compound 234a was synthesized similar to Compound 233a, using (1H-pyrazol-4-yl)boronic acid as starting material, Cs₂CO₃ as base and reacting at 105° C. for 2 h under microwave irradiation in step B.

¹H NMR (400 MHz, DMSO-d₆) δ 12.85 (br.s, 1H), 8.04 (s, 2H), 7.80 (d, J=0.8 Hz, 1H), 7.52-7.62 (m, 3H), 7.47 (dd, J=8.4, 1.6 Hz, 1H), 7.34 (dd, J=8.4, 2.0 Hz, 1H), 6.69-6.85 (m, 3H), 5.04-5.17 (m, 1H), 4.69 (dd, J=15.2, 7.2 Hz, 1H), 4.57 (dd, J=15.2, 3.2 Hz, 1H), 4.44-4.52 (m, 1H), 4.30-4.42 (m, 1H), 3.90 (d, J=13.2 Hz, 1H), 3.75 (d, J=13.2 Hz, 1H), 3.01 (d, J=10.8 Hz, 1H), 2.89 (d, J=11.2 Hz, 1H), 2.60-2.73 (m, 2H), 2.37-2.48 (m, 1H), 2.12-2.29 (m, 2H), 2.02 (s, 3H), 1.63-1.82 (m, 4H).

MS Calcd.: 613.2; MS Found: 614.2 [M+H]⁺.

### Example 111: N-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)nicotinamide (Compound 235a)

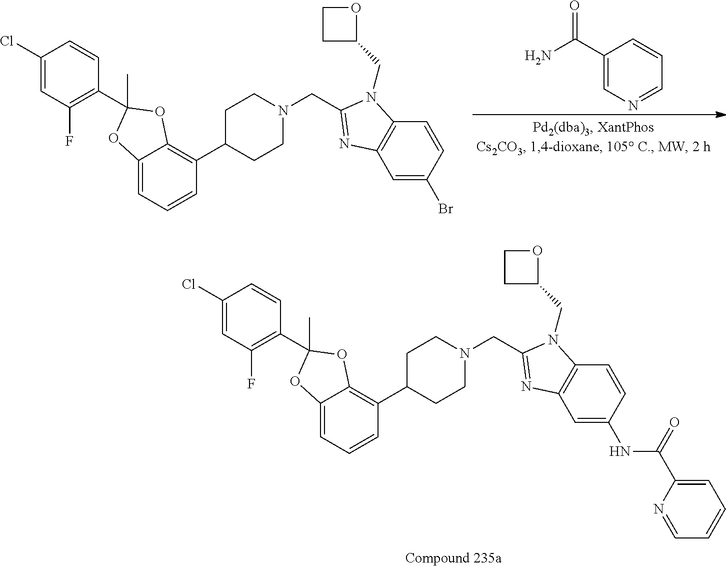 [SMILES: CC1(c2ccc(Cl)cc2F)Oc2cccc(C3CCN(Cc4nc5cc(Br)ccc5n4C[C@@H]4CCO4)CC3)c2O1 ; NC(=O)c1cccnc1 ; CC1(c2ccc(Cl)cc2F)Oc2cccc(C3CCN(Cc4nc5cc(NC(=O)c6ccccn6)ccc5n4C[C@@H]4CCO4)CC3)c2O1]

A mixture of 5-bromo-2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole (216 mg, 0.344 mmol), nicotinamide (42.0 mg, 0.344 mmol), Pd₂(dba)₃ (31.0 mg, 0.0344 mmol), XantPhos (36.0 mg, 0.0688 mmol), and Cs₂CO₃ (243 mg, 0.757 mmol) in anhydrous 1,4-dioxane (3 mL) was degassed and refilled with N₂ for three times. The mixture was stirred at 105° C. for 2 hours under microwave irradiation. The reaction mixture was diluted with H₂O (10 mL) and extracted with EtOAc (20 mL*3). The combined organic phase was dried over Na₂SO₄, and filtered. The filtrate was concentrated and purified by prep-HPLC (0.1% formic acid in water and acetonitrile) to afford N-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)nicotinamide as a white solid (81.3 mg, 36% yield).

¹H NMR (400 MHz, CDCl₃) δ 9.14 (s, 1H), 8.76 (s, 1H), 8.20-8.32 (m, 2H), 7.91 (s, 1H), 7.63-7.69 (m, 1H), 7.40-7.54 (m, 2H), 7.08-7.17 (m, 2H), 6.73-6.80 (m, 1H), 6.62-6.72 (m, 2H), 5.18-5.29 (m, 1H), 4.52-4.80 (m, 3H), 4.34-4.46 (m, 1H), 3.90-4.05 (m, 2H), 2.95-3.08 (m, 2H), 2.66-2.79 (m, 2H), 2.42-2.55 (m, 1H), 2.25-2.40 (m, 2H), 2.05 (s, 3H), 1.74-1.94 (m, 4H).

MS Calcd.: 667.2; MS Found: 668.2 [M+H]⁺.

### Example 112: 2-((4-((S)-2-(4-chloro-2-fluorophenyl)-2-methylbenzo[d][1,3]dioxol-4-yl)piperidin-1-yl)methyl)-5-(5-methylpyrimidin-2-yl)-1-(((S)-oxetan-2-yl)methyl)-1H-benzo[d]imidazole (Compound 236a)

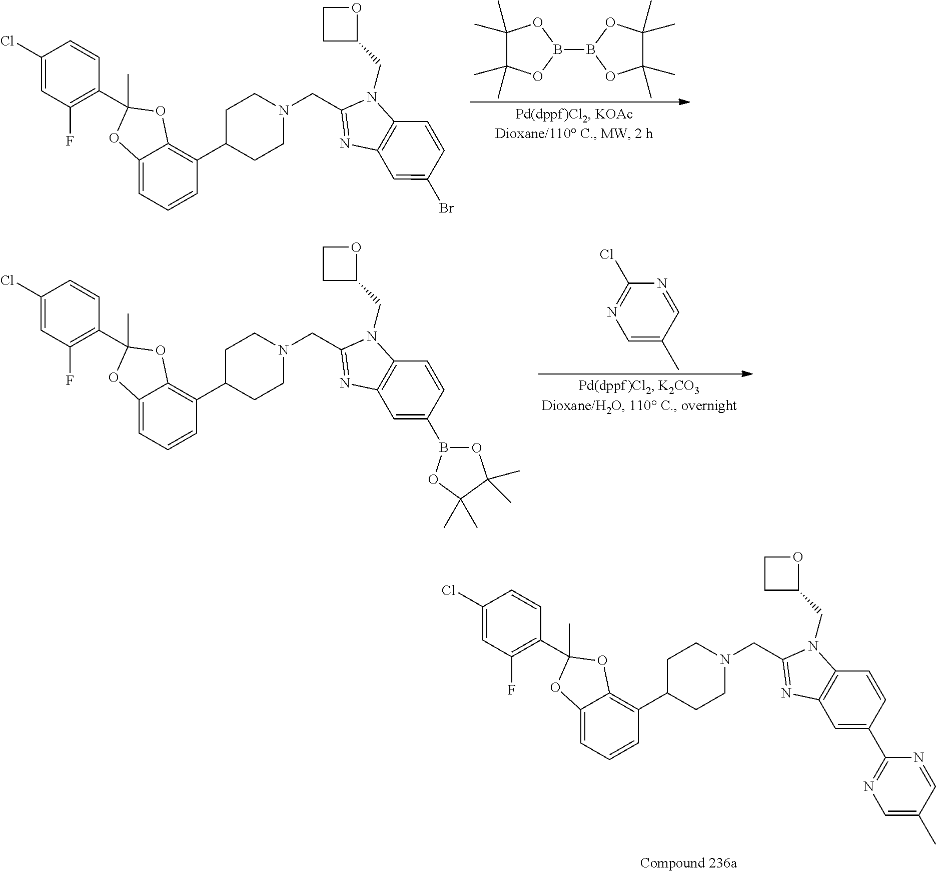

### Step A: 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-(4,4,5,5-tetramethyl-1,3,2-dioxaborolan-2-yl)-1H-benzo[d]imidazole

To a mixture of 5-bromo-2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole (50.0 mg, 0.0800 mmol) in 1,4-dioxane (1.5 mL) were added 4,4,4′,4′,5,5,5′,5′-octamethyl-2,2′-bi(1,3,2-dioxaborolane) (41.0 mg, 0.160 mmol), Pd(dppf)Cl₂ (7.00 mg, 8.00 umol) and KOAc (24.0 mg, 0.240 mmol). The mixture was degassed and refilled with N₂ for three times. Then the mixture was stirred at 110° C. for 2 h under microwave irradiation. The reaction mixture was used directly for the next step.

MS Calcd.: 673.3; MS Found: 674.2 [M+H]⁺.

### Step B: 2-((4-(2-(4-chloro-2-fluorophenyl)-2-methylbenzo[d][1,3]dioxol-4-yl)piperidin-1-yl)methyl)-5-(5-methylpyrimidin-2-yl)-1-(((S)-oxetan-2-yl)methyl)-1H-benzo[d]imidazole

To the solution of the step A were added 2-chloro-5-methylpyrimidine (11.0 mg, 80.0 umol), Pd(dppf)Cl₂ (7.00 mg, 8.00 umol), K₂CO₃ (33.0 mg, 0.240 mmol) and water (0.500 mL). The mixture was degassed and refilled with N₂ for three times. The reaction was stirred at 110° C. overnight. The solvent was evaporated and the residue was purified by Prep-TLC (DCM/MeOH=15/1), and Prep-HPLC (0.1% formic acid in water and acetonitrile) to afford 2-((4-(2-(4-chloro-2-fluorophenyl)-2-methylbenzo[d][1,3]dioxol-4-yl)piperidin-1-yl)methyl)-5-(5-methylpyrimidin-2-yl)-1-(((S)-oxetan-2-yl)methyl)-1H-benzo[d]imidazole (5.00 mg, 10% yield over two steps).

¹H NMR (400 MHz, DMSO-d₆) δ 8.73 (s, 2H), 8.58 (s, 1H), 8.30 (dd, J=8.4, 1.2 Hz, 1H), 7.72 (d, J=8.4 Hz, 1H), 7.50-7.58 (m, 2H), 7.33 (dd, J=8.4, 1.6 Hz, 1H), 6.70-6.82 (m, 3H), 5.10-5.20 (m, 1H), 4.75 (dd, J=15.2, 7.2 Hz, 1H), 4.62 (d, J=15.2 Hz, 1H), 4.35-4.52 (m, 2H), 3.94 (d, J=13.2 Hz, 1H), 3.78 (d, J=13.2 Hz, 1H), 3.02 (d, J=10.4 Hz, 1H), 2.89 (d, J=11.2 Hz, 1H0), 2.60-2.76 (m, 2H), 2.42-2.50 (m, 1H), 2.30 (s, 3H), 2.14-2.32 (m, 2H), 2.02 (s, 3H), 1.68-1.81 (m, 4H).

MS Calcd.: 639.2; MS Found: 640.2 [M+H]⁺.

### Example 113: 2-((4-(2-(4-chloro-2-fluorophenyl)-2-methylbenzo[d][1,3]dioxol-4-yl)piperidin-1-yl)methyl)-5-(2-methylpyrimidin-5-yl)-1-(((S)-oxetan-2-yl)methyl)-1H-benzo[d]imidazole (Compound 237a)

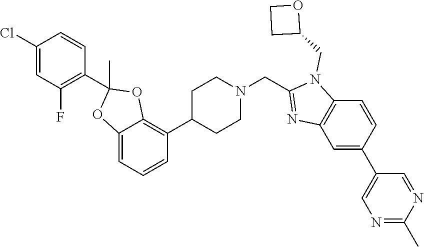 [SMILES: Cc1ncc(-c2ccc3c(c2)nc(CN2CCC(c4cccc5c4OC(C)(c4ccc(Cl)cc4F)O5)CC2)n3C[C@@H]2CCO2)cn1]

Compound 237a was synthesized following the route of Compound 236a, using 5-chloro-2-methylpyrimidine instead of 2-chloro-5-methylpyrimidine.

¹H NMR (400 MHz, DMSO-d₆) δ 9.05 (s, 2H), 8.00 (d, J=1.2 Hz, 1H), 7.78 (d, J=8.8 Hz, 1H), 7.51-7.67 (m, 3H), 7.34 (dd, J=8.4, 2.0 Hz, 1H), 6.70-6.85 (m, 3H), 5.10-5.18 (m, 1H), 4.75 (dd, J=15.2, 7.2 Hz, 1H), 4.57-4.69 (m, 1H), 4.34-4.54 (m, 2H), 3.94 (d, J=13.6 Hz, 1H), 3.81 (d, J=13.2 Hz, 1H), 3.02 (d, J=10.0 Hz, 1H), 2.89 (d, J=10.8 Hz, 1H), 2.60-2.76 (m, 5H), 2.39-2.48 (m, 1H), 2.13-2.30 (m, 2H), 2.03 (s, 3H), 1.61-1.86 (m, 4H).

MS Calcd.: 639.2; MS Found: 640.2 [M+H]⁺.

### Example 114: 2-((4-(2-(4-chloro-2-fluorophenyl)-2-methylbenzo[d][1,3]dioxol-4-yl)piperidin-1-yl)methyl)-5-(1-methyl-1H-1,2,3-triazol-4-yl)-1-(((S)-oxetan-2-yl)methyl)-1H-benzo[d]imidazole (Compound 238a)

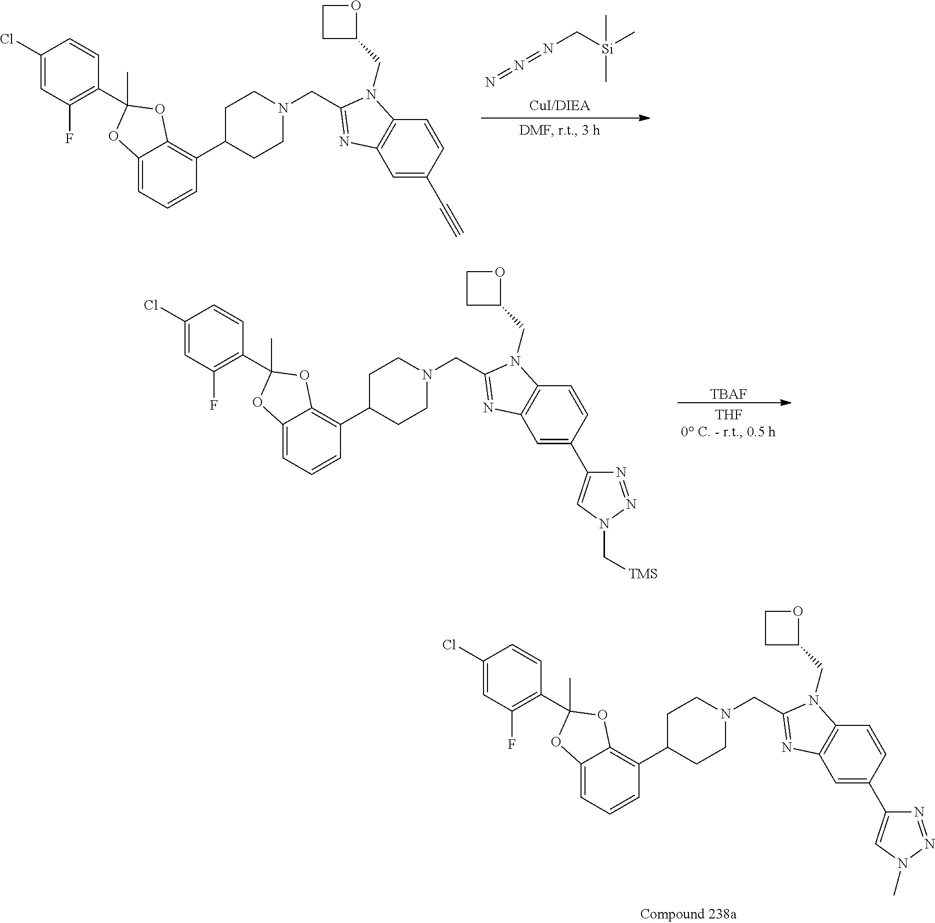

### Step A: 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-(1-((trimethylsilyl)methyl)-1H-1,2,3-triazol-4-yl)-1H-benzo[d]imidazole

To a solution of 2-((4-(2-(4-chloro-2-fluorophenyl)-2-methylbenzo[d][1,3]dioxol-4-yl)piperidin-1-yl)methyl)-5-ethynyl-1-(((S)-oxetan-2-yl)methyl)-1H-benzo[d]imidazole (210 mg, 0.368 mmol) and CuI (7.00 mg, 0.0370 mmol) in DMF (2 mL) were added (azidomethyl)trimethylsilane (142 mg, 1.10 mmol) and DIEA (0.06 mL, 0.368 mmol). The mixture was stirred at room temperature for 3 h. The reaction mixture was diluted with water (10 mL) and extracted with EtOAc (3×20 mL). The combined organic phase was washed with brine (20 mL), dried over anhydrous Na₂SO₄ and concentrated. The residue was purified by flash chromatography (eluted with DCM/MeOH=15/1) to afford 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-(1-((trimethylsilyl)methyl)-1H-1,2,3-triazol-4-yl)-1H-benzo[d]imidazole as a light yellow oil (185 mg, 72% yield).

MS Calcd.: 700.3; MS Found: 701.3 [M+H]⁺.

### Step B: 2-((4-(2-(4-chloro-2-fluorophenyl)-2-methylbenzo[d][1,3]dioxol-4-yl)piperidin-1-yl)methyl)-5-(1-methyl-1H-1,2,3-triazol-4-yl)-1-(((S)-oxetan-2-yl)methyl)-1H-benzo[d]imidazole

To a solution of 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-(1-((trimethylsilyl)methyl)-1H-1,2,3-triazol-4-yl)-1H-benzo[d]imidazole (130 mg, 0.186 mmol) in THF (1 mL) was added TBAF (1 M in THF) (0.22 mL, 0.223 mmol) at 0° C. The mixture was stirred at room temperature for 0.5 h. The reaction mixture was diluted with water (5 mL) and extracted with EtOAc (3×10 mL). The combined organic phase was washed with brine (10 mL), dried over anhydrous Na₂SO₄ and concentrated. The residue was purified by prep-HPLC (0.1% formic acid in water and acetonitrile) to afford 2-((4-(2-(4-chloro-2-fluorophenyl)-2-methylbenzo[d][1,3]dioxol-4-yl)piperidin-1-yl)methyl)-5-(1-methyl-1H-1,2,3-triazol-4-yl)-1-(((S)-oxetan-2-yl)methyl)-1H-benzo[d]imidazole as a white solid (119 mg, 88% yield).

¹H NMR (400 MHz, DMSO-d₆) δ 8.49 (s, 1H), 8.01 (d, J=1.6 Hz, 1H), 7.64-7.75 (m, 2H), 7.51-7.60 (m, 2H), 7.34 (dd, J=8.4, 2.0 Hz, 1H), 6.71-6.83 (m, 3H), 5.07-5.18 (m, 1H), 4.73 (dd, J=15.2, 7.2 Hz, 1H), 4.59 (dt, J=15.2, 3.2 Hz, 1H), 4.33-4.52 (m, 2H), 4.09 (s, 3H), 3.93 (d, J=13.2 Hz, 1H), 3.77 (d, J=13.2 Hz, 1H), 3.02 (d, J=11.2 Hz, 1H), 2.89 (d, J=11.2 Hz, 1H), 2.59-2.75 (m, 2H), 2.38-2.52 (m, 1H), 2.12-2.30 (m, 2H), 2.02 (s, 3H), 1.63-1.83 (m, 4H).

MS Calcd.: 628.2; MS Found: 629.3 [M+H]⁺.

### Example 115: 3-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-1,2,4-oxadiazol-5(4H)-one (Compound 239a); (single isomer 1: Compound 239b; single isomer 2: Compound 239c)

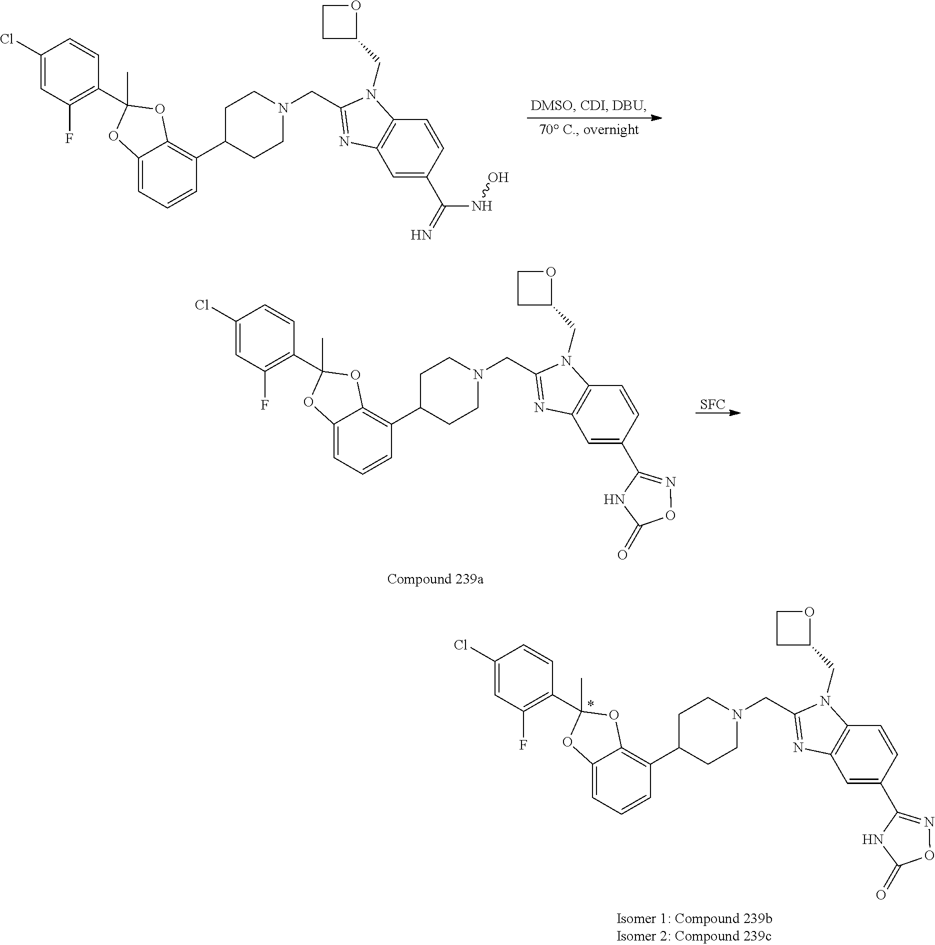

### Step A: 3-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-1,2,4-oxadiazol-5(4H)-one (Compound 239a)

To a mixture of 2-((4-(2-(4-chloro-2-fluorophenyl)-2-methylbenzo[d][1,3]dioxol-4-yl)piperidin-1-yl)methyl)-N-hydroxy-1-(((S)-oxetan-2-yl)methyl)-1H-benzo[d]imidazole-5-carboximidamide (150 mg, 0.247 mmol) in DMSO (6 mL) were added CDI (80.0 mg, 0.495 mmol) and DBU (93.7 mg, 0.617 mmol) at room temperature. The mixture was stirred at 70° C. overnight. The mixture was adjusted to pH=4-5 with HCOOH, and then H₂O was added. The mixture was filtered. The filter cake was dissolved with DCM, dried over Na₂SO₄, and concentrated. The residue was purified with Prep-HPLC (0.1% formic acid in H₂O and MeOH) to afford 3-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-1,2,4-oxadiazol-5(4H)-one as a white solid (53.0 mg, 34% yield).

¹H NMR (400 MHz, CDCl₃) δ 14.64 (br.s, 1H), 8.04 (br.s, 1H), 7.60-7.75 (m, 2H), 7.45-7.55 (m, 1H), 7.03-7.19 (m, 2H), 6.63-6.79 (m, 2H), 6.58 (br.s, 1H), 5.20-5.30 (m, 1H), 4.26-4.92 (m, 4H), 3.73-4.11 (m, 1H), 3.26-3.67 (m, 1H), 2.39-2.85 (m, 4H), 2.01 (s, 3H), 1.35-1.96 (m, 7H).

MS Calcd.: 631.2; MS Found: 632.2 [M+H]⁺.

### Step B: isomer 1: Compound 239b; isomer 2: Compound 239c

3-(2-((4-(2-(4-Chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-1,2,4-oxadiazol-5(4H)-one (Compound 239a) (113 mg, 0.247 mmol) was separated via SFC (Column: ChiralPak AD-H Daicel chemical Industries, Ltd, 250*30 mm I.D., Sum; Mobile phase A: Supercritical CO2, Mobile phase B: IPA (0.1% NH3H2O), A:B=60:40 at 50 ml/min; Column Temp: 38° C.; Nozzle Pressure: 100 Bar; Wavelength: 254 nm.) to afford two compounds. isomer 1: Compound 239b (34.5 mg, white solid), isomer 2: Compound 239c (41.2 mg, white solid).

### Isomer 1: Compound 239b:

SFC Method: Column: AD-H; Column Size: 0.46 cm I.D.*15 cm L; Mobile phase: CO2:IPA (0.1% DEA)=60:40; Flow rate: 2.5 mL; UV: 254 nM; 10 min. Rt: 5.422 min.

¹H NMR (400 MHz, DMSO-d₆) δ 12.88 (br.s, 1H), 8.04 (d, J=1.2 Hz, 1H), 7.81 (d, J=8.4 Hz, 1H), 7.70 (dd, J=8.8, 1.6 Hz, 1H), 7.52-7.62 (m, 2H), 7.34 (dd, J=8.4, 2.0 Hz, 1H), 6.73-6.82 (m, 3H), 5.09-5.14 (m, 1H), 4.77 (dd, J=15.2, 7.2 Hz, 1H), 4.63 (dd, J=15.2, 2.8 Hz, 1H), 4.45-4.50 (m, 1H), 4.36-4.43 (m, 1H), 3.96 (d, J=13.6 Hz, 1H), 3.80 (d, J=13.2 Hz, 1H), 3.02 (d, J=11.2 Hz, 1H), 2.87 (d, J=10.4 Hz, 1H), 2.59-2.73 (m, 2H), 2.38-2.45 (m, 1H), 2.15-2.30 (m, 2H), 2.02 (s, 3H), 1.66-1.81 (m, 4H).

MS Calcd.: 631.2; MS Found: 631.8 [M+H]⁺.

### Isomer 2: Compound 239c:

SFC Method: Column: AD-H; Column Size: 0.46 cm I.D.*15 cm L; Mobile phase: CO2:IPA (0.1% DEA)=60:40; Flow rate: 2.5 mL; UV: 254 nM; 10 min. SFC Rt: 6.237 min.

¹H NMR (400 MHz, DMSO-d₆) δ 12.88 (br.s, 1H), 8.04 (d, J=1.2 Hz, 1H), 7.80 (d, J=8.4 Hz, 1H), 7.69 (dd, J=8.4, 1.2 Hz, 1H), 7.51-7.61 (m, 2H), 7.34 (dd, J=8.4, 2.0 Hz, 1H), 6.73-6.82 (m, 3H), 5.12-5.20 (m, 1H), 4.77 (dd, J=15.2, 7.2 Hz, 1H), 4.62 (dd, J=15.2, 2.8 Hz, 1H), 4.35-4.49 (m, 2H), 3.95 (d, J=13.6 Hz, 1H), 3.79 (d, J=13.6 Hz, 1H), 3.01 (d, J=10.8 Hz, 1H), 2.87 (d, J=11.2 Hz, 1H), 2.60-2.74 (m, 2H), 2.40-2.44 (m, 1H), 2.15-2.30 (m, 2H), 2.02 (s, 3H), 1.67-1.80 (m, 4H)

MS Calcd.: 631.2; MS Found: 631.8 [M+H]⁺.

### Example 116: N-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)acetamide (Compound 240a)

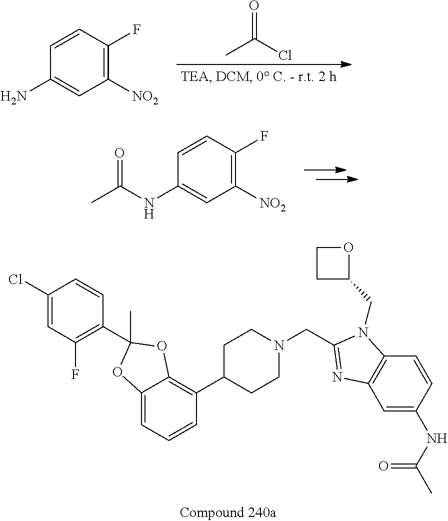

### Step A: N-(4-fluoro-3-nitrophenyl)acetamide

To a solution of 4-fluoro-3-nitroaniline (500 mg, 3.20 mmol) and TEA (390 mg, 3.84 mmol) in DCM (5 mL) was added acetyl chloride (251 mg, 3.20 mmol) at 0° C. The mixture was stirred at room temperature for 2 h. The reaction was diluted with DCM (10 mL), washed with 1N HCl aq. solution (5 mL), water (5 mL), brine (5 mL), and dried over Na₂SO₄. The organic layer was concentrated to give N-(4-fluoro-3-nitrophenyl)acetamide as a yellow solid (650 mg, over 100% yield, crude), which was used in next step without further purification.

MS Calcd.: 198.0; MS Found: 199.2 [M+H]⁺.

### Step B: N-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)acetamide (Compound 240a)

Compound 240a was synthesized following the route of Compound 230 from step A to step D, using N-(4-fluoro-3-nitrophenyl)acetamide and (S)-oxetan-2-ylmethanamine 4-methylbenzenesulfonate as starting material.

¹H NMR (400 MHz, DMSO-d₆) δ 9.87 (s, 1H), 7.89 (d, J=1.6 Hz, 1H), 7.53-7.59 (m, 2H), 7.51 (d, J=8.4 Hz, 1H), 7.31-7.35 (m, 2H), 6.71-6.79 (m, 2H), 6.72-6.76 (m, 1H), 5.06-5.13 (m, 1H), 4.66 (dd, J=15.2, 6.8 Hz, 1H), 4.51-4.55 (m, 1H), 4.44-4.47 (m, 1H), 4.35-4.39 (m, 1H), 3.87 (d, J=13.2 Hz, 1H), 3.72 (d, J=13.2 Hz, 1H), 2.99 (d, J=10.4 Hz, 1H), 2.86 (d, J=11.2 Hz, 1H), 2.63-2.72 (m, 2H), 2.39-2.49 (m, 1H), 2.12-2.25 (m, 2H), 2.04 (s, 3H), 2.02 (s, 3H), 1.66-1.79 (m, 4H).

MS Calcd.: 604.2; MS Found: 605.2 [M+H]⁺.

### Example 117: 2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-5-(1H-tetrazol-5-yl)-1H-benzo[d]imidazole (Compound 241a)

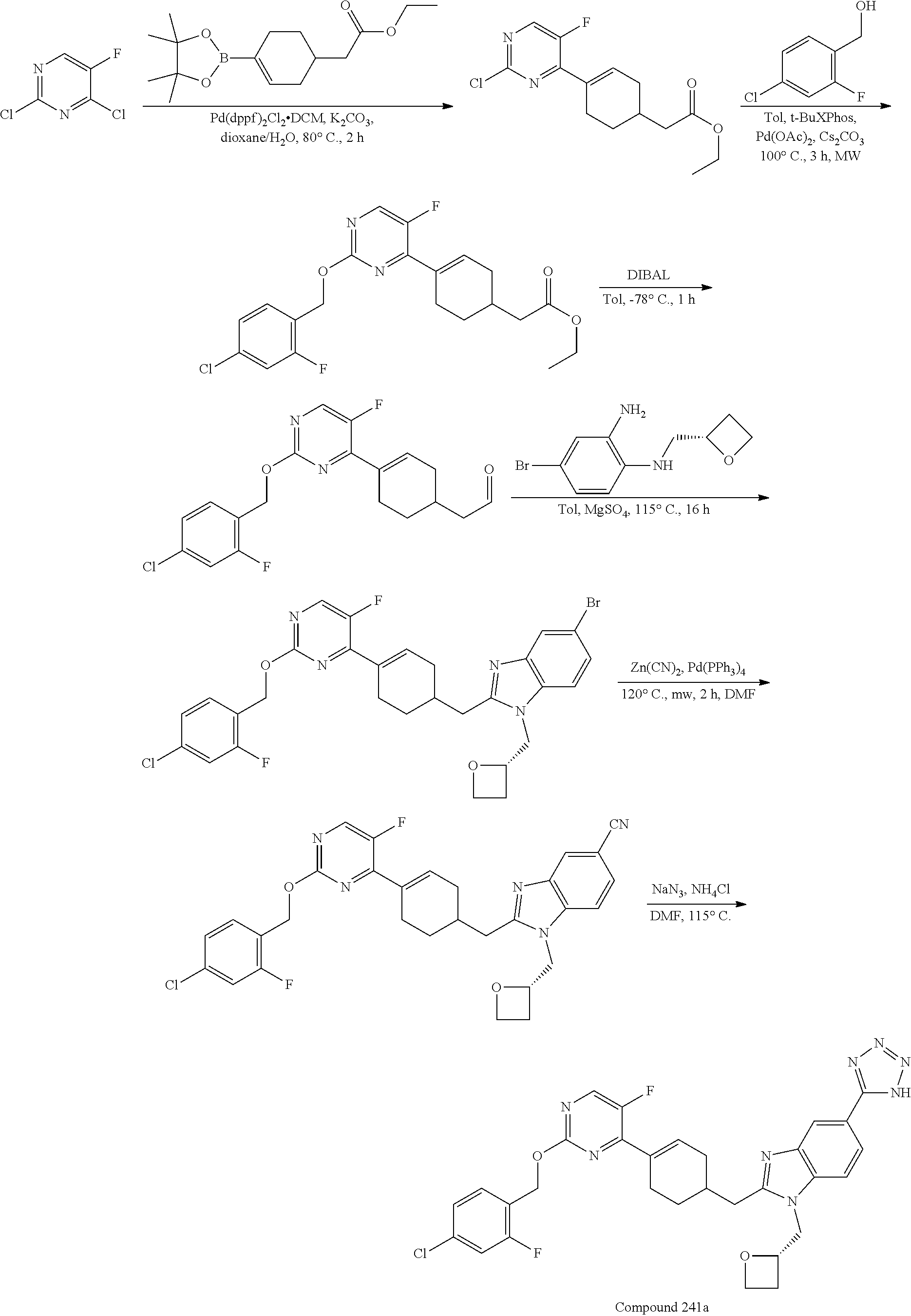

### Step A: ethyl 2-(4-(2-chloro-5-fluoropyrimidin-4-yl)cyclohex-3-en-1-yl)acetate

To a mixture of 2,4-dichloro-5-fluoropyrimidine (800 mg, 4.79 mmol) in dioxane/H₂O (12.5 mL/1.5 mL) were added ethyl 2-(4-(4,4,5,5-tetramethyl-1,3,2-dioxaborolan-2-yl)cyclohex-3-en-1-yl)acetate (1.55 g, 5.27 mmol), K₂CO₃ (2.32 g, 16.8 mmol), and Pd(dppf)Cl₂·DCM (589 mg, 0.720 mmol) at room temperature. The mixture was stirred at 80° C. for 2 hours under N₂. The mixture was filtered, and the filter cake was washed with DCM (30 mL). The filtrate was dried over Na₂SO₄, and concentrated. The residue was purified with silica gel column (PE/EtOAc=5:1) to afford ethyl 2-(4-(2-chloro-5-fluoropyrimidin-4-yl)cyclohex-3-en-1-yl)acetate as a colorless oil (1.30 g, 90% yield).

MS Calcd.: 298.1; MS Found: 299.0 [M+H]⁺.

### Step B: ethyl 2-(4-(2-((4-chloro-2-fluorobenzyl)oxy)-5-fluoropyrimidin-4-yl)cyclohex-3-en-1-yl)acetate

A mixture of ethyl 2-(4-(2-chloro-5-fluoropyrimidin-4-yl)cyclohex-3-en-1-yl)acetate (1.50 g, 5.00 mmol), (4-chloro-2-fluorophenyl)methanol (963 mg, 6.00 mmol), t-BuXPhos (425 mg, 1.00 mmol), Pd(OAc)₂ (112 mg, 0.500 mmol), and Cs₂CO₃ (4.10 g, 12.5 mmol) in anhydrous toluene (12 mL) was stirred at 100° C. under atmosphere of N₂ with microwave irradiation for 3 hours. To the mixture was added H₂O (20 mL) and extracted with EtOAc (40 mL*3). The combined organic phase was dried over Na₂SO₄, and filtered. The filtrate was concentrated and purified by flash chromatography (PE/EtOAc=15/1) to afford ethyl 2-(4-(2-((4-chloro-2-fluorobenzyl)oxy)-5-fluoropyrimidin-4-yl)cyclohex-3-en-1-yl)acetate as a yellow solid (1.29 g, 61% yield).

MS Calcd.: 422.1; MS Found: 423.1 [M+H]⁺.

### Step C: 2-(4-(2-((4-chloro-2-fluorobenzyl)oxy)-5-fluoropyrimidin-4-yl)cyclohex-3-en-1-yl)acetaldehyde

To a solution of ethyl 2-(4-(2-((4-chloro-2-fluorobenzyl)oxy)-5-fluoropyrimidin-4-yl)cyclohex-3-en-1-yl)acetate (445 mg, 1.05 mmol) in anhydrous toluene (20 mL) was added DIBAL-H (1 M in THF) (1.58 mL, 1.58 mmol) dropwise at −78° C. The resulting mixture was stirred at −78° C. for 1 hour. To the mixture were added THE (30 mL) and H₂O (30 mL). The mixture was filtered. The filtrate was concentrated in vacuo and purified by flash chromatography (PE/EtOAc=10/1) to afford 2-(4-(2-((4-chloro-2-fluorobenzyl)oxy)-5-fluoropyrimidin-4-yl)cyclohex-3-en-1-yl)acetaldehyde as a yellow solid (287 mg, 72% yield).

MS Calcd.: 378.1; MS Found: 379.0 [M+H]⁺.

### Step D: 5-bromo-2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-1H-benzo[d]imidazole

A mixture of 2-(4-(2-((4-chloro-2-fluorobenzyl)oxy)-5-fluoropyrimidin-4-yl)cyclohex-3-en-1-yl)acetaldehyde (413 mg, 1.09 mmol), (S)-4-bromo-N¹-(oxetan-2-ylmethyl)benzene-1,2-diamine (336 mg, 1.30 mmol), MgSO₄ (656 mg, 5.45 mmol) in anhydrous toluene (8 mL) was stirred at 115° C. for 16 hours. The reaction mixture was filtered. The filtrate was concentrated and purified by flash chromatography (PE/EtOAc/DCM=10/1/1) to afford 5-bromo-2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-1H-benzo[d]imidazole as a yellow solid (318 mg, 48% yield).

MS Calcd.: 614.1; MS Found: 615.0 [M+H]⁺.

### Step E: 2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-1H-benzo[d]imidazole-5-carbonitrile

A mixture of 5-bromo-2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-1H-benzo[d]imidazole (318 mg, 0.517 mmol), Zn(CN)₂ (607 mg, 5.17 mmol), Pd(PPh₃)₄ (239 mg, 0.207 mmol) in anhydrous DMF (4 mL) was bubbled with N₂. The resulting mixture was stirred at 120° C. with microwave for 2 hours. The mixture was diluted with H₂O (10 mL) and extracted with EtOAc (20 mL*3). The combined organic phase was dried over Na₂SO₄ and filtered. The filtrate was concentrated and purified by prep-HPLC (0.1% NH₄HCO₃ in water and acetonitrile) to afford 2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-1H-benzo[d]imidazole-5-carbonitrile as a white solid (276 mg, 49% yield).

¹H NMR (400 MHz, DMSO-d₆) δ 8.60 (d, J=3.6 Hz, 1H), 8.12 (d, J=0.8 Hz, 1H), 7.80 (d, J=8.4 Hz, 1H), 7.54-7.64 (m, 2H), 7.49 (dd, J=10.0, 1.6 Hz, 1H), 7.31-7.35 (m, 1H), 6.86 (s, 1H), 5.39 (s, 2H), 4.97-5.06 (m, 1H), 4.61-4.70 (m, 1H), 4.41-4.58 (m, 2H), 4.23-4.38 (m, 1H), 2.91-3.09 (m, 2H), 2.50-2.80 (m, 3H), 2.25-2.49 (m, 3H), 2.09-2.21 (m, 1H), 1.92-2.04 (m, 1H), 1.41-1.58 (m, 1H).

MS Calcd.: 561.2; MS Found: 562.2 [M+H]⁺.

### Step F: 2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-5-(1H-tetrazol-5-yl)-1H-benzo[d]imidazole (Compound 241a)

A mixture of 2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-1H-benzo[d]imidazole-5-carbonitrile (65.0 mg, 0.115 mmol), NaN₃ (15.0 mg, 0.230 mmol), NH₄Cl (13.0 mg, 0.230 mmol) in anhydrous DMF (1 mL) was stirred at 115° C. for 16 hours. The mixture was diluted with H₂O (5 mL) and extracted with EtOAc (10 mL*3). The combined organic phase was dried over Na₂SO₄, and filtered. The filtrate was concentrated and purified by prep-HPLC (0.1% HCOOH in water and acetonitrile) to afford 2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-5-(1H-tetrazol-5-yl)-1H-benzo[d]imidazole as a white solid (10.0 mg, 13% yield).

¹H NMR (400 MHz, DMSO-d₆) δ 8.60 (d, J=3.2 Hz, 1H), 8.17 (s, 1H), 7.89 (d, J=8.8 Hz, 1H), 7.67 (d, J=8.4 Hz, 1H), 7.58 (t, J=8.0 Hz, 1H), 7.50 (dd, J=10.0, 2.0 Hz, 1H), 7.33 (dd, J=8.0, 2.0 Hz, 1H), 6.88 (s, 1H), 5.40 (s, 2H), 5.00-5.10 (m, 1H), 4.52-4.65 (m, 1H), 4.42-4.51 (m, 2H), 4.28-4.40 (m, 1H), 2.93-3.05 (m, 3H), 2.56-2.75 (m, 3H), 2.30-2.44 (m, 2H), 2.09-2.22 (m, 1H), 1.95-2.05 (m, 1H), 1.40-1.60 (m, 1H).

MS Calcd.: 604.2; MS Found: 605.2 [M+H]⁺.

### Example 118: 3-(2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-1H-benzo[d]imidazol-5-yl)-1,2,4-oxadiazol-5(4H)-one (Compound 242a)

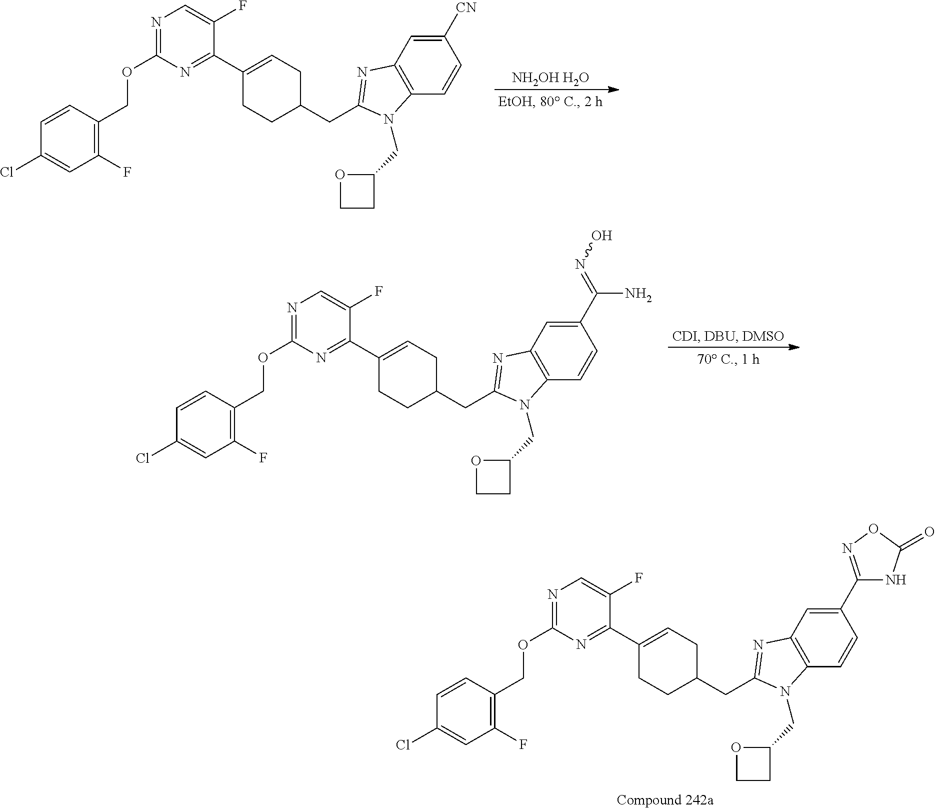

### Step A: 2-((4-(2-((4-chloro-2-fluorobenzyl)oxy)-5-fluoropyrimidin-4-yl) cyclohex-3-en-1-yl)methyl)-N′-hydroxy-1-(((S)-oxetan-2-yl)methyl)-1H-benzo[d]imidazole-5-carboximidamide

To a solution of 2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-1H-benzo[d]imidazole-5-carbonitrile (110 mg, 0.196 mmol) in ethanol (4 mL) was added hydroxylamine (50% in H₂O) (132 mg, 1.96 mmol). The resulting mixture was stirred at 80° C. for 2 hours. The reaction mixture was concentrated and purified by flash chromatography (DCM:CH₃OH=15:1) to give 2-((4-(2-((4-chloro-2-fluorobenzyl)oxy)-5-fluoropyrimidin-4-yl)cyclohex-3-en-1-yl)methyl)-N′-hydroxy-1-(((S)-oxetan-2-yl)methyl)-1H-benzo[d] imidazole-5-carboximidamide (47.0 mg, 40% yield).

MS Calcd.: 594.2; MS Found: 595.1 [M+H]⁺.

### Step B: 3-(2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-1H-benzo[d]imidazol-5-yl)-1,2,4-oxadiazol-5(4H)-one (Compound 242a)

To a mixture of 2-((4-(2-((4-chloro-2-fluorobenzyl)oxy)-5-fluoropyrimidin-4-yl)cyclohex-3-en-1-yl)methyl)-N′-hydroxy-1-(((S)-oxetan-2-yl)methyl)-1H-benzo[d]imidazole-5-carboximidamide (47.0 mg, 0.0790 mmol) in anhydrous DMSO (4 mL) were added CDI (128 mg, 0.790 mmol), and DBU (120 mg, 0.790 mmol). The resulting mixture was stirred at 70° C. for 1 hour. The reaction mixture was diluted with H₂O (5 mL) and extracted with EtOAc (10 mL*3). The combined organic phase was dried over Na₂SO₄, and filtered. The filtrate was concentrated and purified by prep-HPLC (0.1% HCOOH in water and acetonitrile) to afford 3-(2-((4-(2-((4-chloro-2-fluorobenzyl)oxy)-5-fluoropyrimidin-4-yl)cyclohex-3-en-1-yl)methyl)-1-(((S)-oxetan-2-yl)methyl)-1H-benzo[d]imidazol-5-yl)-1,2,4-oxadiazol-5(4H)-one as a white solid (13.4 mg, 28% yield).

¹H NMR (400 MHz, DMSO-d₆) δ 8.60 (d, J=3.6 Hz, 1H), 8.24 (s, 1H), 7.99 (s, 1H), 7.67 (s, 2H), 7.58 (t, J=8.0 Hz, 1H), 7.50 (dd, J=10.0, 1.6 Hz, 1H), 7.33 (dd, J=8.0, 1.6 Hz, 1H), 6.87 (s, 1H), 5.40 (s, 2H), 4.98-5.08 (m, 1H), 4.60 (dd, J=15.6, 6.4 Hz, 1H), 4.42-4.51 (m, 2H), 4.27-4.36 (m, 1H), 2.93-3.03 (m, 2H), 2.54-2.73 (m, 3H), 2.30-2.45 (m, 3H), 2.06-2.23 (m, 1H), 1.93-2.06 (m, 1H), 1.43-1.58 (m, 1H).

MS Calcd.: 620.2; MS Found: 621.2 [M+H]⁺.

### Example 119: 2-({4-[2-(4-chloro-2-fluorophenyl)-2-methyl-2H-1,3-benzodioxol-4-yl]piperidin-1-yl}methyl)-5-(5-methyl-4H-1,2,4-triazol-3-yl)-1-{[(2S)-oxetan-2-yl]methyl}-1H-1,3-benzodiazole (Compound 243a)

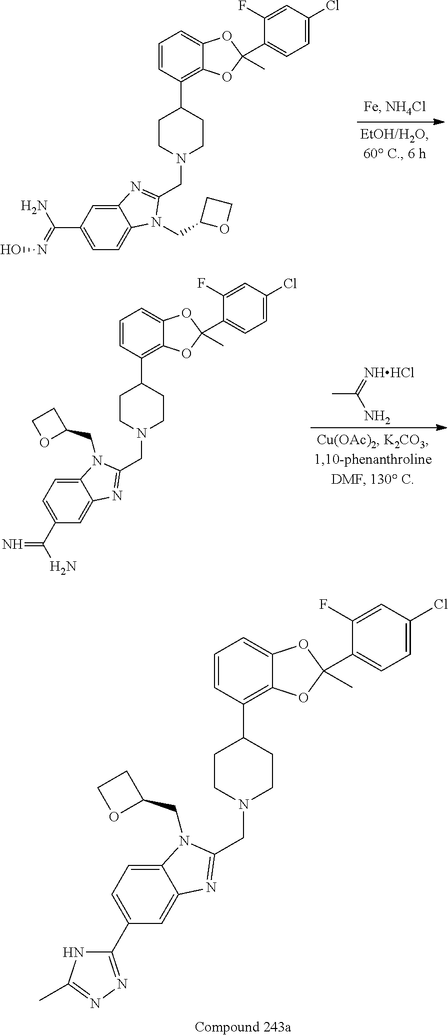

### Step A: 2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazole-5-carboximidamide

A mixture of 2-((4-(2-(4-chloro-2-fluorophenyl)-2-methylbenzo[d][1,3]dioxol-4-yl)piperidin-1-yl)methyl)-N′-hydroxy-1-((S)-oxetan-2-ylmethyl)-1H-benzo[d]imidazole-5-carboximidamide (400 mg, 0.66 mmol), iron powder (370 mg, 6.62 mmol), ammonium chloride (360 mg, 6.62 mmol) in H₂O (1.2 mL) and ethanol (5 mL) was stirred at 60° C. for 6 hours. After cooling down to room temperature, the mixture was filtered and filtrate was purified by prep-HPLC to furnish 2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazole-5-carboximidamide (180 mg, yield: 46.3%) as a white solid.

MS Calcd.: 589.2; MS Found: 590.3 [M+H]⁺.

### Step B: 2-({4-[2-(4-chloro-2-fluorophenyl)-2-methyl-2H-1,3-benzodioxol-4-yl]piperidin-1-yl}methyl)-5-(5-methyl-4H-1,2,4-triazol-3-yl)-1-{[(2S)-oxetan-2-yl]methyl}-1H-1,3-benzodiazole (Compound 243a)

A mixture of 2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazole-5-carboximidamide (42 mg, 0.06 mmol), acetimidamide hydrochloride (12 mg, 0.12 mmol), Cu(OAc)₂ (5 mg, 0.02 mmol), K₂CO₃ (67 mg, 0.48 mmol) and 1,10-phenanthroline (2 mg, 0.01 mmol) in DMF (2 ml) was stirred at 130° C. under N₂ for 8 hours, then, an additional portion of 2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazole-5-carboximidamide (42 mg, 0.06 mmol) was added and the reaction was stirred at 130° C. under N₂ for an additional 8 hrs, followed by addition of the third portion of 2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazole-5-carboximidamide (42 mg, 0.06 mmol). The mixture was stirred at 130° C. under N₂ for an additional 32 hours. After cooled down to room temperature, the reaction mixture was filtered and the filter cake was wash with DCM. The filtrate was concentrated under reduced pressure. The residue was purified by prep-HPLC to give Compound 243a (1.02 mg, yield: 0.7%) as a white solid.

MS Calcd.: 628.2; MS Found: 629.2 [M+H]⁺.

¹H NMR (400 MHz, MeOD): 8.26 (s, 1H), 7.94 (d, J=8.0 Hz, 1H), 7.75-7.70 (m, 1H), 7.61-7.54 (m, 1H), 7.30-7.24 (m, 1H), 7.21 (d, J=8.0 Hz, 1H), 6.79-6.68 (m, 3H), 5.35-5.25 (m, 2H), 4.76-4.60 (m, 2H), 4.52-4.45 (m, 1H), 4.02-3.85 (m, 2H), 3.09-3.03 (m, 1H), 2.95-2.90 (m, 1H), 2.88-2.75 (m, 1H), 2.74-2.60 (m, 1H), 2.60-2.50 (m, 1H), 2.48 (s, 3H), 2.36-2.20 (m, 2H), 2.03 (s, 3H), 1.98-1.75 (m, 4H).

¹⁹FNMR: −112.34, −112.41.

### Example 120: 4-[2-(4-chloro-2-fluorophenyl)-2-methyl-2H-1,3-benzodioxol-4-yl]-1-({6-[5-(difluoromethyl)-4H-1,2,4-triazol-3-yl]-3-{[(2S)-oxetan-2-yl]methyl}-3H-imidazo[4,5-b]pyridin-2-yl}methyl)piperidine (Compound 244a)

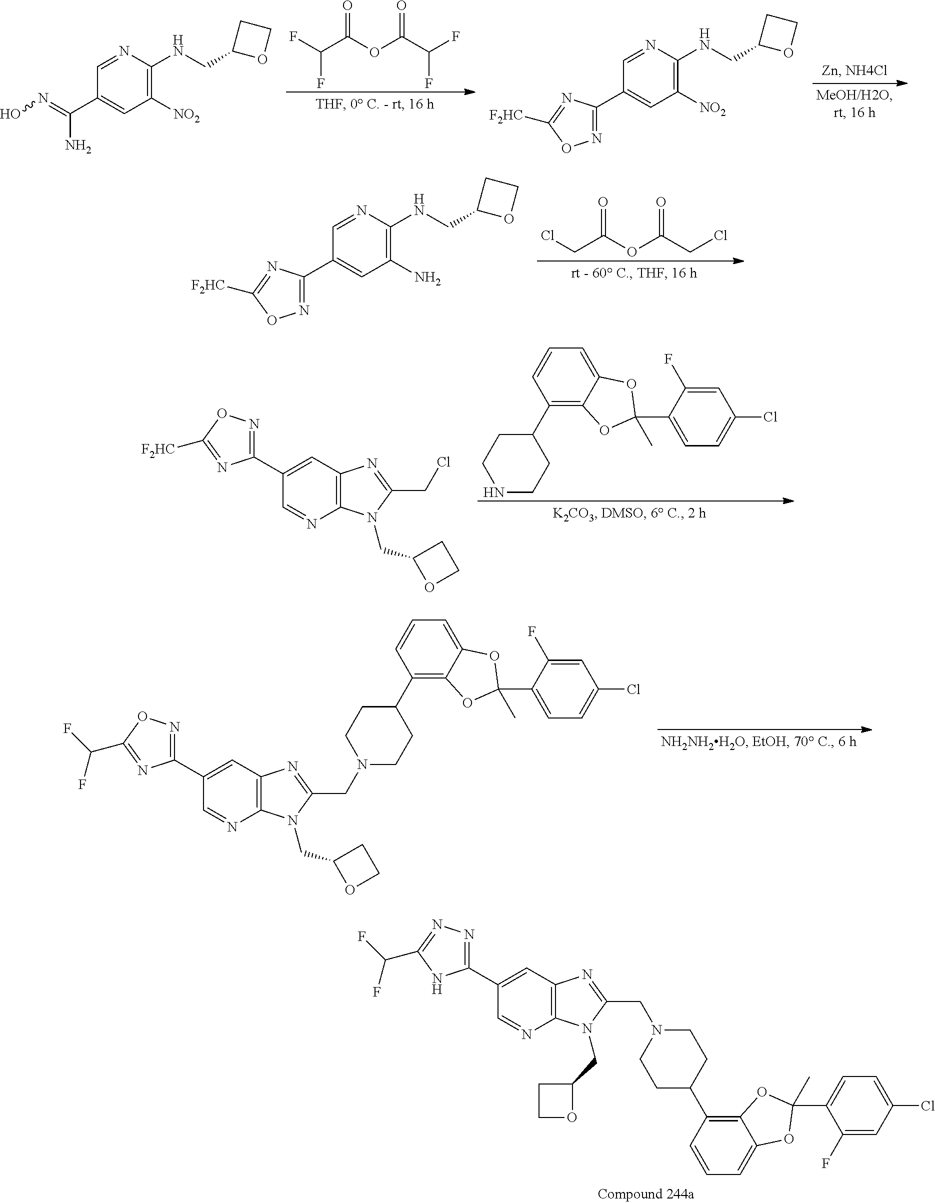

### Step A: (S)-5-(5-(difluoromethyl)-1,2,4-oxadiazol-3-yl)-3-nitro-N-(oxetan-2-ylmethyl)pyridin-2-amine

To a solution of (S)—N′-hydroxy-5-nitro-6-((oxetan-2-ylmethyl)amino)nicotinimidamide (200 mg, 0.749 mmol) in THF (10 mL) were added dropwise 2,2-difluoroacetic anhydride (652 mg, 3.745 mmol) in THF (2 mL) at 0° C. The mixture was stirred at room temperature for 16 hours. The reaction mixture was poured into cold water (50 mL) and extracted with EtOAc (2×50 mL), the combined organic layer was washed with brine, dried over sodium sulfate, filtered and concentrated under reduced pressure. The residue was purified by silica gel column chromatography to furnish (S)-5-(5-(difluoromethyl)-1,2,4-oxadiazol-3-yl)-3-nitro-N-(oxetan-2-ylmethyl)pyridin-2-amine (100 mg, yield: 40.8%) as yellow oil.

MS Calcd.: 327.1; MS Found: 328.2 [M+H]⁺.

### Step B: (S)-5-(5-(difluoromethyl)-1,2,4-oxadiazol-3-yl)-N2-(oxetan-2-ylmethyl)pyridine-2,3-diamine

A mixture of (S)-5-(5-(difluoromethyl)-1,2,4-oxadiazol-3-yl)-3-nitro-N-(oxetan-2-ylmethyl)pyridin-2-amine (100 mg, 0.306 mmol) and zinc powder (199 mg, 3.06 mmol) in ammonium chloride aqueous solution (5 mL) and methanol (5 mL) was stirred at room temperature for 16 hours. The mixture was poured into cold water (30 mL) and extracted with DCM (2×30 mL), the combined organic layer was washed with brine, dried over sodium sulfate, filtered to furnish (S)-5-(5-(difluoromethyl)-1,2,4-oxadiazol-3-yl)-N2-(oxetan-2-ylmethyl)pyridine-2,3-diamine (90 mg) as yellow oil.

MS Calcd.: 297.1; MS Found: 298.1 [M+H]⁺.

### Step C: (S)-3-(2-(chloromethyl)-3-(oxetan-2-ylmethyl)-3H-imidazo[4,5-b]pyridin-6-yl)-5-(difluoromethyl)-1,2,4-oxadiazole

To a solution of (S)-5-(5-(difluoromethyl)-1,2,4-oxadiazol-3-yl)-N2-(oxetan-2-ylmethyl)pyridine-2,3-diamine (90 mg, 0.303 mmol) in THE (10 mL) were added 2-chloroacetic anhydride (57 mg, 0.333 mmol) at 0° C. The mixture was stirred at room temperature for 2 hours and 60° C. for 16 h. The reaction mixture was poured into cold water (30 mL) and extracted with DCM (2×30 mL), the combined organic layer was washed with brine, dried over sodium sulfate, filtered and concentrated under reduced pressure. The residue was purified by silica gel column chromatography to furnish (S)-3-(2-(chloromethyl)-3-(oxetan-2-ylmethyl)-3H-imidazo[4,5-b]pyridin-6-yl)-5-(difluoromethyl)-1,2,4-oxadiazole (20 mg, yield: 18.6%) as yellow solid.

MS Calcd.: 355.1; MS Found: 356.1 [M+H]⁺.

### Step D: 3-(2-((4-(2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-b]pyridin-6-yl)-5-(difluoromethyl)-1,2,4-oxadiazole

A mixture of (S)-3-(2-(chloromethyl)-3-(oxetan-2-ylmethyl)-3H-imidazo[4,5-b]pyridin-6-yl)-5-(difluoromethyl)-1,2,4-oxadiazole (20 mg, 0.056 mmol), 4-(2-(4-chloro-2-fluorophenyl)-2-methylbenzo[d][1,3]dioxol-4-yl)piperidine (22 mg) and K₂CO₃ (23 mg, 0.168 mmol) in DMSO (3 mL) was stirred at 60° C. for 2 hours. The mixture was purified by column chromatography to furnish 3-(2-((4-(2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-b]pyridin-6-yl)-5-(difluoromethyl)-1,2,4-oxadiazole (15 mg, yield: 40%) as white solid.

MS Calcd.: 666.2; MS Found: 667.2 [M+H]⁺.

### Step E: 4-[2-(4-chloro-2-fluorophenyl)-2-methyl-2H-1,3-benzodioxol-4-yl]-1-({6-[5-(difluoromethyl)-4H-1,2,4-triazol-3-yl]-3-{[(2S)-oxetan-2-yl]methyl}-3H-imidazo[4,5-b]pyridin-2-yl}methyl)piperidine (Compound 244a)

A mixture of 3-(2-((4-(2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-b]pyridin-6-yl)-5-(difluoromethyl)-1,2,4-oxadiazole (15 mg, 0.0225 mmol) and NH₂NH₂·H₂O (0.2 mL) in EtOH (1 mL) was stirred at 70° C. for 6 hours. The reaction mixture was purified by prep-HPLC to give Compound 244a (1.5 mg, yield: 10%) as white solid.

MS Calcd.: 665.2; MS Found: 666.2 [M+H]⁺.

¹H NMR (400 MHz, MeOD) 8.99 (d, J=2.0 Hz, 1H), 8.54 (d, J=2.0 Hz, 1H), 7.49 (t, J=8.4 Hz, 1H), 7.18 (d, J=10.8 Hz, 1H), 7.11 (d, J=8.0 Hz, 1H), 6.80-6.58 (m, 4H), 5.27-5.21 (m, 3H), 4.56-4.32 (m, 2H), 3.93 (s, 2H), 2.97-2.91 (m, 2H), 2.72-2.55 (m, 2H), 2.50-2.45 (m, 1H), 2.27-2.17 (m, 2H), 1.93 (s, 3H), 1.87-1.77 (m, 2H), 1.75-1.65 (m, 2H).

¹⁹FNMR −112.37, −112.38, −114.85.

### Example 121: 4-[2-(4-chloro-2-fluorophenyl)-2-methyl-2H-1,3-benzodioxol-4-yl]-1-[(3-{[(2R)-oxolan-2-yl]methyl}-6-[5-(trifluoromethyl)-4H-1,2,4-triazol-3-yl]-3H-imidazo[4,5-b]pyridin-2-yl)methyl]piperidine (Compound 245a)

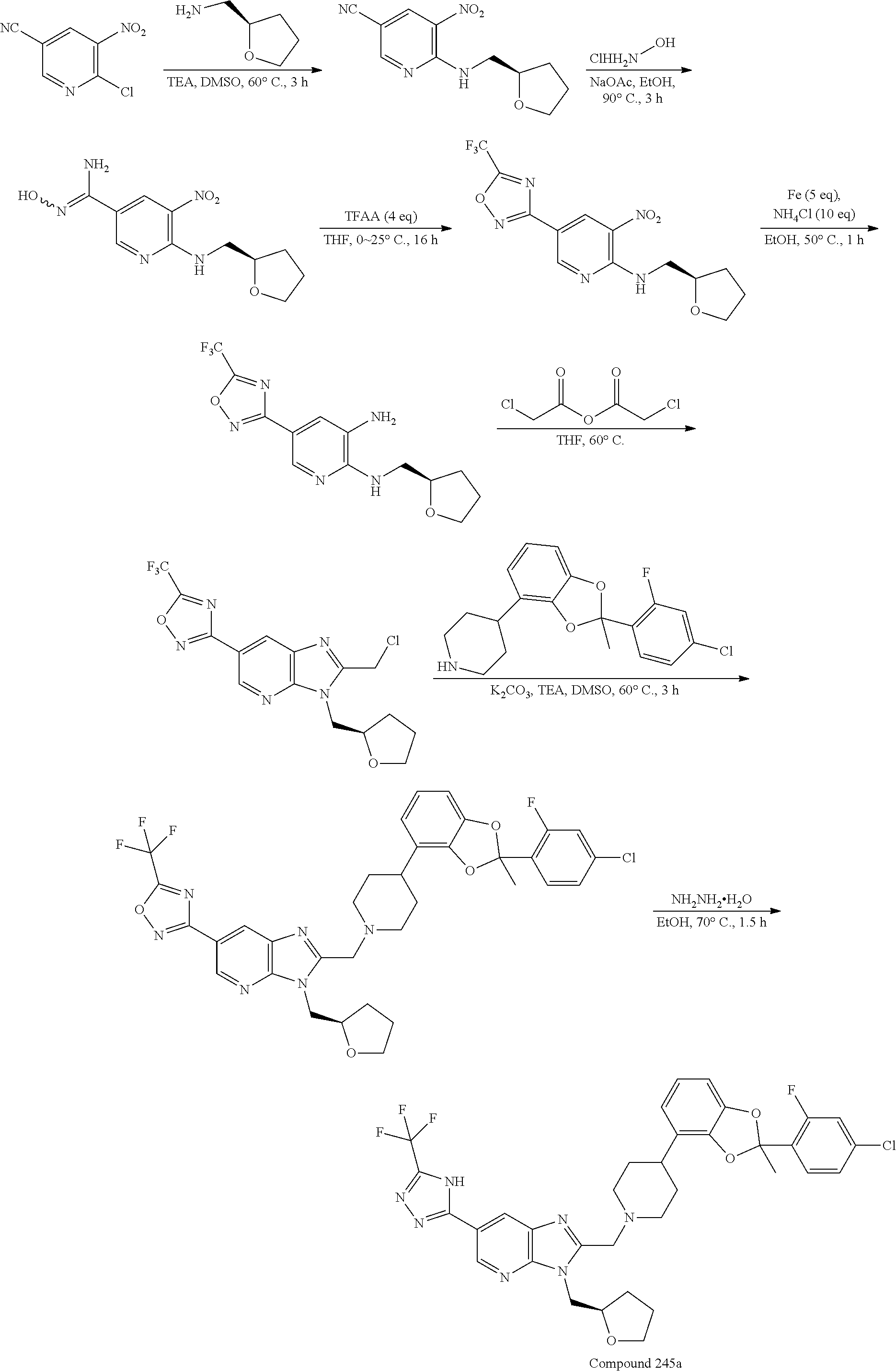

### Step A: (R)-5-nitro-6-(((tetrahydrofuran-2-yl)methyl)amino)nicotinonitrile

A mixture of 6-chloro-5-nitronicotinonitrile (7.0 g, 31.8 mmol), (R)-(tetrahydrofuran-2-yl)methanamine (5.8 g, 31.8 mmol) and TEA (664 mg, 6.57 mmol) in DMSO (6 mL) was stirred at 60° C. for 3 hours. After the reaction was completed, the reaction was diluted with EA, washed with aq NaHCO3 and brine, dried over sodium sulfate, concentrated to give a crude. The crude was purified by column chromatography (PE:EA=5/1) to give (R)-5-nitro-6-(((tetrahydrofuran-2-yl)methyl)amino)nicotinonitrile (530 mg, 97.7.% yield) as yellow solid.

MS Calcd.: 248.1; MS Found: 249.1 [M+H]⁺.

### Step B: (R)—N′-hydroxy-5-nitro-6-(((tetrahydrofuran-2-yl)methl)amino) nicotinimidamide

A mixture of (R)-5-nitro-6-(((tetrahydrofuran-2-yl)methyl)amino)nicotinonitrile (530 mg, 2.14 mmol), hydroxylamine hydrochloride (1.47 g, 21.4 mmol) and NaOAc (2.11 g, 25.7 mmol) in EtOH (15 mL) was stirred at 90° C. for 3 hours. After the reaction was completed, the reaction was concentrated in vacuum, purified by column chromatography (DCM/MeOH=100/1-50/1) to give (R)—N′-hydroxy-5-nitro-6-(((tetrahydrofuran-2-yl)methyl)amino)nicotinimidamide (595 mg, 77.400 yield) as orange solid.

MS Calcd.: 281.1; MS Found: 282.1 [M+H]⁺.

### Step C: (R)-3-nitro-N-((tetrahydrofuran-2-yl)methyl)-5-(5-(trifluoromethyl)-1,2,4-oxadiazol-3-yl)pyridin-2-amine

To a solution of (R)—N′-hydroxy-5-nitro-6-(((tetrahydrofuran-2-yl)methyl)amino)nicotinimidamide (595 mg, 1.66 mmol) in THE (80 mL) was dropwise added trifluoroacetic anhydride (1.74 g, 8.29 mmol) in THE (2 mL) under Ar at 0° C. The reaction was stirred at RT for 16 hours. After the reaction was completed, the reaction was diluted with EA, washed with aq NaHCO₃ and brine, dried over sodium sulfate, concentrated, purified by column chromatography (PE:EA=3/1) to give (R)-3-nitro-N-((tetrahydrofuran-2-yl)methyl)-5-(5-(trifluoromethyl)-1,2,4-oxadiazol-3-yl)pyridin-2-amine (597 mg, 78.8% yield) as yellow solid.

MS Calcd.: 359.1; MS Found: 360.0 [M+H]⁺.

### Step D: (R)—N2-((tetrahydrofuran-2-yl)methyl)-5-(5-(trifluoromethyl)-1,2,4-oxadiazol-3-yl)pyridine-2,3-diamine

To a solution of (R)-3-nitro-N-((tetrahydrofuran-2-yl)methyl)-5-(5-(trifluoromethyl)-1,2,4-oxadiazol-3-yl)pyridin-2-amine (200 mg, 0.56 mmol) and NH4Cl (300 mg, 5.6 mmol) in EtOH/H2O (4 mL/2 mL) was stirred at 0° C. for 15 min, then Fe (156 mg, 2.78 mmol) was added, the mixture was stirred at 50° C. for 1 hour.

After the reaction was completed, the reaction was concentrated in vacuum, purified by column chromatography (PE:EA=1/1) to give (R)—N2-((tetrahydrofuran-2-yl)methyl)-5-(5-(trifluoromethyl)-1,2,4-oxadiazol-3-yl)pyridine-2,3-diamine (92 mg, 50% yield) as orange solid.

MS Calcd.: 329.1; MS Found: 330.1 [M+H]⁺.

### Step E: (R)-3-(2-(chloromethyl)-3-((tetrahydrofuran-2-yl)methyl)-3H-imidazo[4,5-b]pyridin-6-yl)-5-(trifluoromethyl)-1,2,4-oxadiazole

To a solution of (R)—N2-((tetrahydrofuran-2-yl)methyl)-5-(5-(trifluoromethyl)-1,2,4-oxadiazol-3-yl)pyridine-2,3-diamine (92 mg, 0.28 mmol) in THE (6 mL) was dropwise added 2-chloroacetic anhydride (51 mg, 0.30 mmol) at RT, the mixture was stirred at RT for 1 hour. Then the mixture was stirred at 60° C. for 16 hours. 2-chloroacetic anhydride (51 mg, 0.30 mmol) in THE (1 mL) was dropwise added, the mixture was stirred at 60° C. for another 16 hours. The reaction was diluted with EA, washed with aq NaHCO₃ and brine, dried over sodium sulfate, concentrated in vacuum, purified by prep-TLC (PE:EA=4:1) to give (R)-3-(2-(chloromethyl)-3-((tetrahydrofuran-2-yl)methyl)-3H-imidazo[4,5-b]pyridin-6-yl)-5-(trifluoromethyl)-1,2,4-oxadiazole (70 mg, 64.3% yield) as orange oil.

MS Calcd.: 387.1; MS Found: 388.0 [M+H]⁺.

### Step F: 3-(2-((4-(2-(4-chloro-2-fluorophenyl)-2-methylbenzo[d][1,3]dioxol-4-yl)piperidin-1-yl)methyl)-3-(((R)-tetrahydrofuran-2-yl)methyl)-3H-imidazo[4,5-b]pyridin-6-yl)-5-(trifluoromethyl)-1,2,4-oxadiazole

To a solution of 4-(2-(4-chloro-2-fluorophenyl)-2-methylbenzo[d][1,3]dioxol-4-yl)piperidine (69 mg) and TEA (55 mg, 0.54 mmol) in DMSO (5 mL) was added K₂CO₃ (75 mg, 0.54 mmol) and (R)-3-(2-(chloromethyl)-3-((tetrahydrofuran-2-yl)methyl)-3H-imidazo[4,5-b]pyridin-6-yl)-5-(trifluoromethyl)-1,2,4-oxadiazole (70 mg, 0.18 mmol), the mixture was stirred at 60° C. for 3 hours. Upon cooling down, the reaction was diluted with EA, washed with brine, dried over sodium sulfate, concentrated in vacuum, purified by prep-TLC (PE:EA=1/1) to give 3-(2-((4-(2-(4-chloro-2-fluorophenyl)-2-methylbenzo[d][1,3]dioxol-4-yl)piperidin-1-yl)methyl)-3-(((R)-tetrahydrofuran-2-yl)methyl)-3H-imidazo[4,5-b]pyridin-6-yl)-5-(trifluoromethyl)-1,2,4-oxadiazole (37 mg, 29% yield) as a orange oil.

MS Calcd.: 698.2; MS Found: 699.3 [M+H]⁺.

### Step G: 4-[2-(4-chloro-2-fluorophenyl)-2-methyl-2H-1,3-benzodioxol-4-yl]-1-[(3-{[(2R)-oxolan-2-yl]methyl}-6-[5-(trifluoromethyl)-4H-1,2,4-triazol-3-yl]-3H-imidazo[4,5-b]pyridin-2-yl)methyl]piperidine (Compound 245a)

To a solution of 3-(2-((4-(2-(4-chloro-2-fluorophenyl)-2-methylbenzo[d][1,3]dioxol-4-yl)piperidin-1-yl)methyl)-3-(((R)-tetrahydrofuran-2-yl)methyl)-3H-imidazo[4,5-b]pyridin-6-yl)-5-(trifluoromethyl)-1,2,4-oxadiazole (37 mg, 0.053 mmol) in EtOH (3 mL) was added NH₂NH₂ monohydrate (0.5 mL), the mixture was stirred at 70° C. for 1.5 hours. The mixture was filtered, the filtrate was purified by Prep-HPLC (0.1% NH₃H₂O) to give Compound 245a (15.09 mg, yield: 51.6%) as white solid.

MS Calcd.: 697.2; MS Found: 698.4 [M+H]⁺.

¹H NMR (400 MHz, MeOD) δ 9.05 (dd, J₁=0.4 Hz, J₂=2.0 Hz, 1H), 8.59 (d, J=2.0 Hz, 1H), 7.57 (t, J=9.2 Hz, 1H), 7.28 (dd, J₁=1.6 Hz, J₂=10.8 Hz, 1H), 7.21 (d, J=8.4 Hz, 1H), 6.79-6.68 (m, 3H), 4.79-4.64 (m, 2H), 4.52-4.46 (m, 1H), 4.15 (d, J=13.6 Hz, 1H), 3.95-3.89 (m, 2H), 3.76-3.71 (m, 1H), 3.13-3.04 (m, 1H), 3.03-2.96 (m, 1H), 2.78-2.67 (m, 1H), 2.41-2.25 (m, 2H), 2.20-2.08 (m, 1H), 2.02 (s, 3H), 1.98-1.75 (m, 7H). ¹⁹F-NMR: 6-66.34, −112.34.

### Example 122: 4-[2-(4-chloro-2-fluorophenyl)-2-methyl-2H-1,3-benzodioxol-4-yl]-1-[(3-{[(2S)-oxolan-2-yl]methyl}-6-[5-(trifluoromethyl)-4H-1,2,4-triazol-3-yl]-3H-imidazo[4,5-b]pyridin-2-yl)methyl]piperidine (Compound 246a)

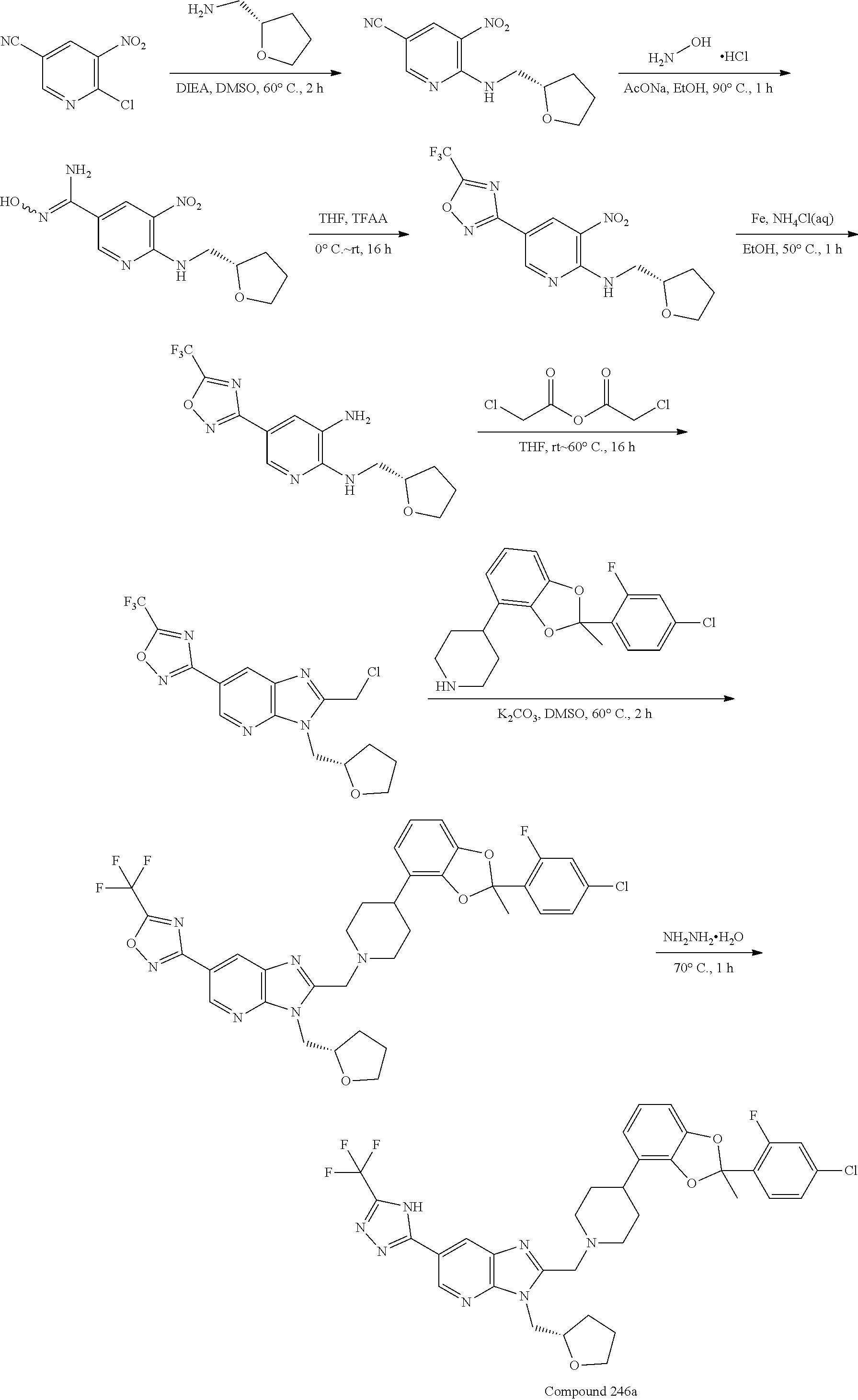

### Step A: (S)-5-nitro-6-(((tetrahydrofuran-2-yl)methyl)amino)nicotinonitrile

A mixture of 6-chloro-5-nitronicotinonitrile (250 mg, 1.37 mmol), (S)-(tetrahydrofuran-2-yl)methanamine (151.8 mg, 1.50 mmol) and DIPEA (1.76 g, 13.66 mmol) in DMSO (5 mL) was stirred at 60° C. for 2 hours. The mixture was poured into cold water (20 mL) and extracted with EtOAc (2×30 mL), the combined organic layer was washed with brine, dried over sodium sulfate, filtered and the residue was purified by column chromatography to furnish (S)-5-nitro-6-(((tetrahydrofuran-2-yl)methyl)amino)nicotinonitrile (320 mg, yield: 83.4%) as yellow solid.

MS Calcd.: 248.1; MS Found: 249.1 [M+H]⁺.

### Step B: (S)—N′-hydroxy-5-nitro-6-(((tetrahydrofuran-2-yl)methyl)amino) nicotinimidamide

A mixture of (S)-5-nitro-6-(((tetrahydrofuran-2-yl)methyl)amino) nicotinonitrile (320 mg, 1.29 mmol), hydroxylamine hydrochloride (890.3 mg, 12.90 mmol), and NaOAc (1.27 g, 15.48 mmol) in ethanol (5 mL) was stirred at 90° C. for 1 hours. The mixture was concentrated under reduced pressure. The residue was purified by column chromatography to furnish (S)—N′-hydroxy-5-nitro-6-(((tetrahydrofuran-2-yl)methyl)amino) nicotinimidamide (300 mg, yield: 82.7%) as yellow oil.

MS Calcd.: 281.1; MS Found: 282.1 [M+H]⁺.

### Step C: (S)-3-nitro-N-((tetrahydrofuran-2-yl)methyl)-5-(5-(trifluoromethyl)-1,2,4-oxadiazol-3-yl)pyridin-2-amine

To a solution of (S)—N′-hydroxy-5-nitro-6-(((tetrahydrofuran-2-yl)methyl)amino)nicotinimidamide (300 mg, 0.97 mmol) in THF (10 mL) were added dropwise trifluoroacetic anhydride (1.02 g, 4.87 mmol) in THE (2 mL) at 0° C. The mixture was stirred at room temperature for 16 hours. The reaction mixture was poured into cold water (20 mL) and extracted with EtOAc (2×30 mL), the combined organic layer was washed with brine, dried over sodium sulfate, filtered, and concentrated under reduced pressure. The residue was purified by column chromatography to furnish (S)-5-(5-(difluoromethyl)-1,2,4-oxadiazol-3-yl)-3-nitro-N-(oxetan-2-ylmethyl)pyridin-2-amine (260 mg, yield: 74.3%) as yellow solid.

MS Calcd.: 359.1; MS Found: 360.0 [M+H]⁺.

### Step D: (S)—N2-((tetrahydrofuran-2-yl)methyl)-5-(5-(trifluoromethyl)-1,2,4-oxadiazol-3-yl)pyridine-2,3-diamine

A mixture of (S)-5-(5-(difluoromethyl)-1,2,4-oxadiazol-3-yl)-3-nitro-N-(oxetan-2-ylmethyl)pyridin-2-amine (260 mg, 0.72 mmol) and iron powder (202.8 mg, 3.62 mmol), ammonium chloride (391.1 mg, 7.24 mmol) in H₂O (2 mL) and ethanol (4 mL) was stirred at 50° C. for 1 hours. The mixture was concentrated under reduced pressure. The residue was purified by column chromatography to furnish (S)—N2-((tetrahydrofuran-2-yl)methyl)-5-(5-(trifluoromethyl)-1,2,4-oxadiazol-3-yl)pyridine-2,3-diamine (80 mg, yield: 33.6%) as yellow solid.

MS Calcd.: 329.1; MS Found: 330.1 [M+H]⁺.

### Step E: (S)-3-(2-(chloromethyl)-3-((tetrahydrofuran-2-yl)methyl)-3H-imidazo[4,5-b] pyridin-6-yl)-5-(trifluoromethyl)-1,2,4-oxadiazole

To a solution of (S)—N2-((tetrahydrofuran-2-yl)methyl)-5-(5-(trifluoromethyl)-1,2,4-oxadiazol-3-yl) pyridine-2,3-diamine (80 mg, 0.24 mmol) in THE (10 mL) were added 2-chloroacetic anhydride (41.6 mg, 0.24 mmol) at room temperature and was stirred at room temperature for 30 mins, then stirred at 60° C. for 16 h. The reaction mixture was poured into cold water (10 mL) and extracted with DCM (2×20 mL), the combined organic layer was washed with brine, dried over sodium sulfate, filtered and concentrated under reduced pressure. The residue was purified by silica gel column chromatography to furnish (S)-3-(2-(chloromethyl)-3-((tetrahydrofuran-2-yl)methyl)-3H-imidazo[4,5-b]pyridin-6-yl)-5-(trifluoromethyl)-1,2,4-oxadiazole (60 mg, yield: 63.8%) as yellow solid.

MS Calcd.: 387.1; MS Found: 388.0 [M+H]⁺.

### Step F: 3-(2-((4-(2-(4-chloro-2-fluorophenyl)-2-methylbenzo[d][1,3]dioxol-4-yl) piperidin-1-yl)methyl)-3-(((S)-tetrahydrofuran-2-yl)methyl)-3H-imidazo[4,5-b]pyridin-6-yl)-5-(trifluoromethyl)-1,2,4-oxadiazole

A mixture of (S)-3-(2-(chloromethyl)-3-((tetrahydrofuran-2-yl)methyl)-3H-imidazo[4,5-b] pyridin-6-yl)-5-(trifluoromethyl)-1,2,4-oxadiazole (60 mg, 0.16 mmol), 4-(2-(4-chloro-2-fluorophenyl)-2-methylbenzo[d][1,3]dioxol-4-yl)piperidine (59.2 mg) and K₂CO₃ (64.2 mg, 0.47 mmol) in DMSO (3 mL) was stirred at 60° C. for 2 hours. The mixture was purified by column chromatography to furnish 3-(2-((4-(2-(4-chloro-2-fluorophenyl)-2-methylbenzo[d][1,3]dioxol-4-yl)piperidin-1-yl)methyl)-3-(((S)-tetrahydrofuran-2-yl)methyl)-3H-imidazo[4,5-b]pyridin-6-yl)-5-(trifluoromethyl)-1,2,4-oxadiazole (50 mg, yield: 46.3%) as white solid.

MS Calcd.: 698.2; MS Found: 699.3 [M+H]⁺.

### Step G: 4-[2-(4-chloro-2-fluorophenyl)-2-methyl-2H-1,3-benzodioxol-4-yl]-1-[(3-{[(2S)-oxolan-2-yl]methyl}-6-[5-(trifluoromethyl)-4H-1,2,4-triazol-3-yl]-3H-imidazo[4,5-b]pyridin-2-yl)methyl]piperidine (Compound 246a)

A mixture of 3-(2-((4-(2-(4-chloro-2-fluorophenyl)-2-methylbenzo[d][1,3]dioxol-4-yl)piperidin-1-yl) methyl)-3-(((S)-tetrahydrofuran-2-yl)methyl)-3H-imidazo[4,5-b]pyridin-6-yl)-5-(trifluoromethyl)-1,2,4-oxadiazole (50 mg, 0.07 mmol) and NH₂NH₂·H₂O (0.5 mL) in EtOH (1 ml) was stirred at 70° C. for 1 hours. The reaction mixture was purified by prep-HPLC to give Compound 246a_(17.95 mg, yield: 36.0%) as white solid.

MS Calcd.: 697.2; MS Found: 698.2 [M+H]⁺.

¹H NMR (400 MHz, MeOD): 9.09-9.06 (m, 1H), 8.61 (d, J=2 Hz, 1H), 7.58 (t, J=8.8 Hz, 1H), 7.30-7.17 (m, 2H), 6.79-6.67 (m, 3H), 4.76-4.61 (m, 2H), 4.51-4.43 (m, 1H), 4.11 (d, J=13.6 Hz, 1H), 3.94-3.87 (m, 2H), 3.77-3.70 (m, 1H), 3.07 (d, J=10.8 Hz, 1H), 2.98 (d, J=10.8 Hz, 1H), 2.74-2.64 (m, 1H), 2.39-2.24 (m, 2H), 2.17-2.09 (m, 1H), 2.02 (s, 3H), 1.97-1.86 (m, 4H), 1.86-1.76 (m, 3H).

### Example 123: 4-[2-(4-chloro-2-fluorophenyl)-2-methyl-2H-1,3-benzodioxol-4-yl]-1-[(3-{[(2S)-oxolan-2-yl]methyl}-6-[5-(trifluoromethyl)-4H-1,2,4-triazol-3-yl]-3H-imidazo[4,5-b]pyridin-2-yl)methyl]piperidine (Compound 247a)

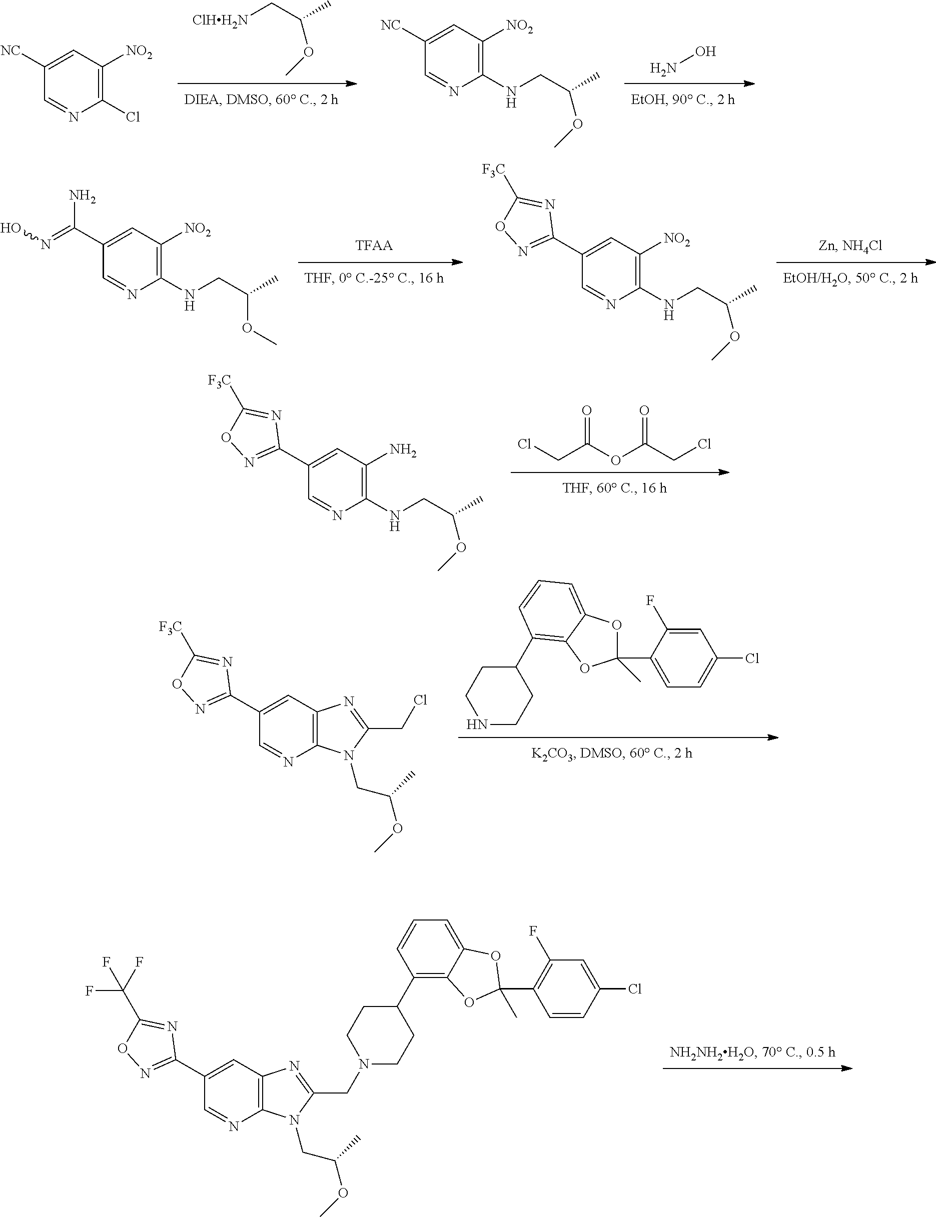

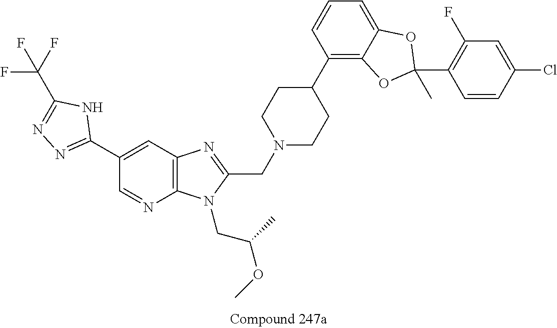 [SMILES: CO[C@@H](C)Cn1c(CN2CCC(c3cccc4c3OC(C)(c3ccc(Cl)cc3F)O4)CC2)nc2cc(-c3nnc(C(F)(F)F)[nH]3)cnc21]

### Step A: (S)-6-((2-methoxypropyl)amino)-5-nitronicotinonitrile

A mixture of 6-chloro-5-nitronicotinonitrile (300 mg, 1.64 mmol), (S)-2-methoxypropan-1-amine hydrochloride (227 mg, 1.80 mmol) and DIPEA (2.1 g, 16.39 mmol) in DMSO (20 mL) was stirred at 60° C. for 2 hours. The mixture was poured into cold water (50 mL) and extracted with EtOAc (2×50 mL), the combined organic layer was washed with brine, dried over sodium sulfate, filtered and the residue was purified by column chromatography to give (S)-6-((2-methoxypropyl)amino)-5-nitronicotinonitrile (290 mg, yield: 74.6%) as yellow solid.

MS Calcd.: 236.1; MS Found: 237.1 [M+H]⁺.

### Step B: (S)—N′-hydroxy-6-((2-methoxypropyl)amino)-5-nitronicotinimidamide

A mixture of (S)-6-((2-methoxypropyl)amino)-5-nitronicotinonitrile (290 mg, 1.23 mmol), hydroxylamine hydrochloride (848 mg, 12.29 mmol), and NaOAc (1.21 g, 14.75 mmol) in ethanol (5 mL) was stirred at 90° C. for 2 hours. The mixture was poured into cold water (50 mL) and extracted with EtOAc (2×50 mL), the combined organic layer was washed with brine, dried over sodium sulfate, filtered to give (S)—N′-hydroxy-6-((2-methoxypropyl)amino)-5-nitronicotinimidamide (260 mg, yield: 73.0%) as yellow oil.

MS Calcd.: 269.1; MS Found: 270.1 [M+H]⁺.

### Step C: (S)—N-(2-methoxypropyl)-3-nitro-5-(5-(trifluoromethyl)-1,2,4-oxadiazol-3-yl)pyridin-2-amine

To a solution of (S)—N′-hydroxy-6-((2-methoxypropyl)amino)-5-nitronicotinimidamide (260 mg, 0.97 mmol) in THE (10 mL) were added dropwise trifluoroacetic anhydride (1.01 g, 4.83 mmol) in THE (2 mL) at 0° C. The mixture was stirred at room temperature for 16 hours. The reaction mixture was poured into cold water (50 mL) and extracted with EtOAc (2×50 mL), the combined organic layer was washed with brine, dried over sodium sulfate, filtered and concentrated under reduced pressure. The residue was purified by silica gel column chromatography to give (S)—N-(2-methoxypropyl)-3-nitro-5-(5-(trifluoromethyl)-1,2,4-oxadiazol-3-yl)pyridin-2-amine (210 mg, yield: 62.7%) as yellow oil.

MS Calcd.: 347.1; MS Found: 348.1 [M+H]⁺.

### Step D: (S)—N2-(2-methoxypropyl)-5-(5-(trifluoromethyl)-1,2,4-oxadiazol-3-yl)pyridine-2,3-diamine

A mixture of (S)—N-(2-methoxypropyl)-3-nitro-5-(5-(trifluoromethyl)-1,2,4-oxadiazol-3-yl)pyridin-2-amine (210 mg, 0.605 mmol) and zinc powder (393 mg, 6.052 mmol) in ammonium chloride aqueous solution (5 mL) and EtOH (5 mL) was stirred at 50° C. for 2 hours. The mixture was poured into cold water (30 mL) and extracted with DCM (2×30 mL), the combined organic layer was washed with brine, dried over sodium sulfate, filtered and concentrated, the residue was purified by silica gel column chromatography to to give (S)—N2-(2-methoxypropyl)-5-(5-(trifluoromethyl)-1,2,4-oxadiazol-3-yl)pyridine-2,3-diamine (125 mg, yield: 65.2%) as yellow solid.

MS Calcd.: 317.1; MS Found: 318.2 [M+H]⁺.

### Step E: (S)-3-(2-(chloromethyl)-3-(2-methoxypropyl)-3H-imidazo[4,5-b]pyridin-6-yl)-5-(trifluoromethyl)-1,2,4-oxadiazole

To a solution of (S)—N2-(2-methoxypropyl)-5-(5-(trifluoromethyl)-1,2,4-oxadiazol-3-yl)pyridine-2,3-diamine (125 mg, 0.394 mmol) in THE (10 mL) were added 2-chloroacetic anhydride (73 mg, 0.426 mmol) at 0° C. The mixture was stirred at room temperature for 2 hours and 60° C. for 16 h. The reaction mixture was poured into cold water (30 mL) and extracted with DCM (2×30 mL), the combined organic layer was washed with brine, dried over sodium sulfate, filtered and concentrated under reduced pressure. The residue was purified by silica gel column chromatography to give (S)-3-(2-(chloromethyl)-3-(2-methoxypropyl)-3H-imidazo[4,5-b]pyridin-6-yl)-5-(trifluoromethyl)-1,2,4-oxadiazole (55 mg, yield: 37.2%) as yellow solid.

MS Calcd.: 375.1; MS Found: 376.0 [M+H]⁺.

### Step F: 3-(2-((4-(2-(4-chloro-2-fluorophenyl)-2-methylbenzo[d][1,3]dioxol-4-yl)piperidin-1-yl)methyl)-3-((S)-2-methoxypropyl)-3H-imidazo[4,5-b]pyridin-6-yl)-5-(trifluoromethyl)-1,2,4-oxadiazole

A mixture of (S)-3-(2-(chloromethyl)-3-(2-methoxypropyl)-3H-imidazo[4,5-b]pyridin-6-yl)-5-(trifluoromethyl)-1,2,4-oxadiazole (55 mg, 0.080 mmol), 4-(2-(4-chloro-2-fluorophenyl)-2-methylbenzo[d][1,3]dioxol-4-yl)piperidine (31 mg) and K₂CO₃ (33 mg, 0.241 mmol) in DMSO (5 mL) was stirred at 60° C. for 2 hours. The mixture was purified by column chromatography to give 3-(2-((4-(2-(4-chloro-2-fluorophenyl)-2-methylbenzo[d][1,3]dioxol-4-yl)piperidin-1-yl)methyl)-3-((S)-2-methoxypropyl)-3H-imidazo[4,5-b]pyridin-6-yl)-5-(trifluoromethyl)-1,2,4-oxadiazole (25 mg, yield: 24.9%) as white solid.

MS Calcd.: 686.2; MS Found: 687.3 [M+H]⁺.

### Step G: 4-[2-(4-chloro-2-fluorophenyl)-2-methyl-2H-1,3-benzodioxol-4-yl]-1-[(3-{[(2S)-oxolan-2-yl]methyl}-6-[5-(trifluoromethyl)-4H-1,2,4-triazol-3-yl]-3H-imidazo[4,5-b]pyridin-2-yl)methyl]piperidine (Compound 247a)

A mixture of 3-(2-((4-(2-(4-chloro-2-fluorophenyl)-2-methylbenzo[d][1,3]dioxol-4-yl)piperidin-1-yl)methyl)-3-((S)-2-methoxypropyl)-3H-imidazo[4,5-b]pyridin-6-yl)-5-(trifluoromethyl)-1,2,4-oxadiazole (25 mg, 0.036 mmol) and NH₂NH₂·H₂O (0.2 mL) in EtOH (1.5 ml) was stirred at 70° C. for 0.5 hours. The reaction mixture was purified by prep-HPLC to give Compound 247a (3.2 mg, yield: 12.8%) as white solid.

MS Calcd.: 685.2; MS Found: 686.2 [M+H]⁺.

¹H NMR (400 MHz, MeOD): 9.06 (d, J=1.6 Hz, 1H), 8.60 (d, J=8.8 Hz, 2H), 7.60-7.55 (m, 1H), 7.29-7.26 (m, 1H), 7.24-7.18 (m, 1H), 6.79-6.68 (m, 3H), 4.71-4.65 (m, 1H), 4.60-4.53 (m, 1H), 4.15 (d, J=13.6 Hz, 1H), 4.01-3.92 (m, 1H), 3.84 (d, J=13.6 Hz, 1H), 3.21 (d, J=3.2 Hz, 3H), 3.17-3.08 (m, 1H), 2.99-2.95 (m, 1H), 2.74-2.67 (m, 1H), 2.40-2.26 (m, 2H), 2.02 (s, 3H), 2.00-1.70 (m, 4H), 1.30-1.24 (m, 3H). 19F-NMR: −65.89, −112.30, −112.31.

### Example 124: 2-({4-[2-(4-chloro-2-fluorophenyl)-2-methyl-2H-1,3-benzodioxol-4-yl]piperidin-1-yl}methyl)-6-fluoro-1-{[(2S)-oxetan-2-yl]methyl}-5-[5-(trifluoromethyl)-4H-1,2,4-triazol-3-yl]-1H-1,3-benzodiazole (Compound 248a)

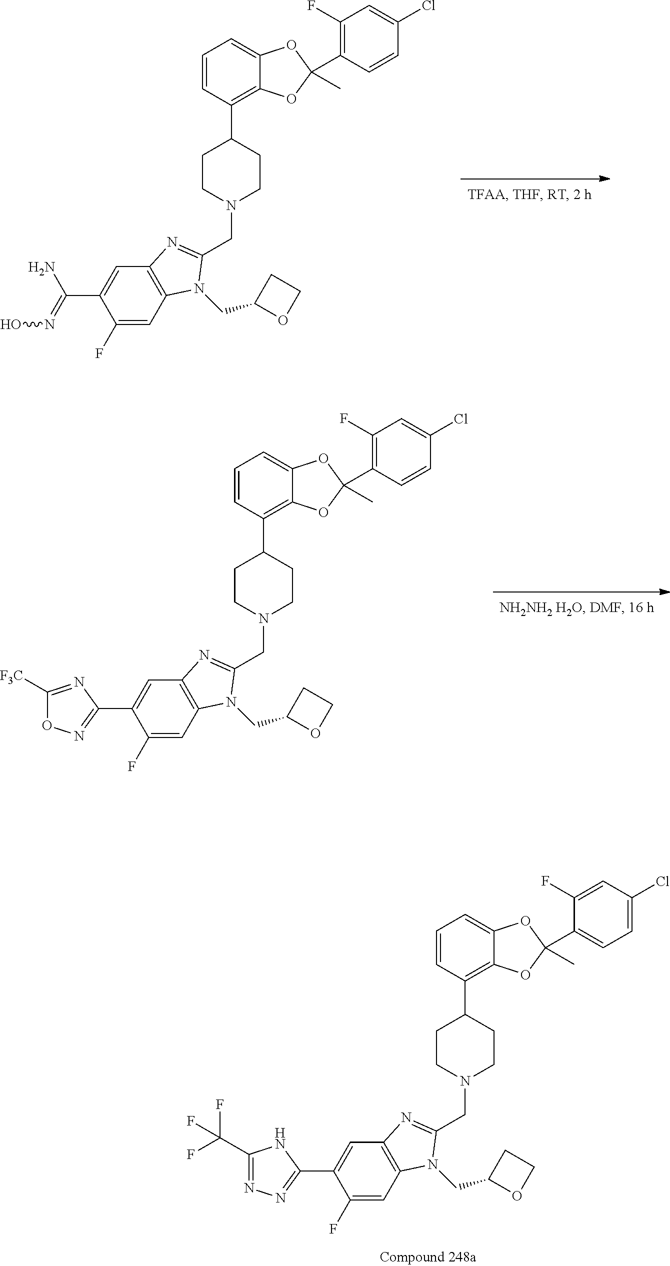 [SMILES: CC1(c2ccc(Cl)cc2F)Oc2cccc(C3CCN(Cc4nc5cc(C(N)=NO)c(F)cc5n4C[C@@H]4CCO4)CC3)c2O1 ; CC1(c2ccc(Cl)cc2F)Oc2cccc(C3CCN(Cc4nc5cc(-c6noc(C(F)(F)F)n6)c(F)cc5n4C[C@@H]4CCO4)CC3)c2O1 ; CC1(c2ccc(Cl)cc2F)Oc2cccc(C3CCN(Cc4nc5cc(-c6nnc(C(F)(F)F)[nH]6)c(F)cc5n4C[C@@H]4CCO4)CC3)c2O1]

### Step A: 3-(2-((4-(2-(4-chloro-2-fluorophenyl)-2-methylbenzo[d][1,3]dioxol-4-yl)piperidin-1-yl)methyl)-6-fluoro-1-(((S)-oxetan-2-yl)methyl)-1H-benzo[d]imidazol-5-yl)-5-(trifluoromethyl)-1,2,4-oxadiazole

A mixture of 2-((4-(2-(4-chloro-2-fluorophenyl)-2-methylbenzo[d][1,3]dioxol-4-yl)piperidin-1-yl)methyl)-6-fluoro-N′-hydroxy-1-(((S)-oxetan-2-yl)methyl)-1H-benzo[d]imidazole-5-carboximidamide (50 mg, 0.08 mmol) and trifluoroacetic anhydride (50 mg, 0.24 mmol) in THE (3 ml) was stirred at RT for 2 hours. After the reaction was completed, the mixture was diluted with EA (20 ml), washed with H₂O (10 ml×3), adjusted to pH=7 with sodium bicarbonate solution. The organic layer was dried over anhydrous sodium sulfate, filtered and evaporated to dryness to give 3-(2-((4-(2-(4-chloro-2-fluorophenyl)-2-methylbenzo[d][1,3]dioxol-4-yl)piperidin-1-yl)methyl)-6-fluoro-1-(((S)-oxetan-2-yl)methyl)-1H-benzo[d]imidazol-5-yl)-5-(trifluoromethyl)-1,2,4-oxadiazole (25 mg, yield: 50%).

MS Calcd.: 701.2; MS Found: 702.2 [M+H]⁺.

### Step B: 2-({4-[2-(4-chloro-2-fluorophenyl)-2-methyl-2H-1,3-benzodioxol-4-yl]piperidin-1-yl}methyl)-6-fluoro-1-{[(2S)-oxetan-2-yl]methyl}-5-[5-(trifluoromethyl)-4H-1,2,4-triazol-3-yl]-1H-1,3-benzodiazole (Compound 248a)

A mixture of 3-(2-((4-(2-(4-chloro-2-fluorophenyl)-2-methylbenzo[d][1,3]dioxol-4-yl)piperidin-1-yl)methyl)-6-fluoro-1-(((S)-oxetan-2-yl)methyl)-1H-benzo[d]imidazol-5-yl)-5-(trifluoromethyl)-1,2,4-oxadiazole (25 mg) and hydrazine hydrate (21 mg, 0.42 mmol) in DMF (2 ml) was stirred at R^{T} for 16 hours. After the reaction was completed, the reaction mixture was purified by prep-HPLC directly to give 2-({4-[2-(4-chloro-2-fluorophenyl)-2-methyl-2H-1,3-benzodioxol-4-yl]piperidin-1-yl}methyl)-6-fluoro-1-{[(2S)-oxetan-2-yl]methyl}-5-[5-(trifluoromethyl)-4H-1,2,4-triazol-3-yl]-1H-1,3-benzodiazole (1.04 mg, yield: 1%) as white solid.

MS Calcd.: 700.2; MS Found: 701.3 [M+H]⁺.

¹H NMR (400 MHz, DMSO-d₆): δ 8.13 (d, 1H), 7.77 (d, J=7.2 Hz, 1H), 7.52-7.60 (m, 2H), 7.31-7.35 (m, 1H)), 6.72-6.81 (m, 3H), 5.08-5.19 (m, 1H), 4.72-4.80 (m, 1H), 4.58-4.65 (m, 1H), 4.40-4.52 (m, 2H), 3.95 (d, J=13.6 Hz, 1H), 3.78 (d, J=13.2 Hz, 1H), 2.97-3.05 (m, 1H), 2.84-2.90 (m, 1H), 2.60-2.75 (m, 2H), 2.40-2.55 (m, 1H), 2.13-2.31 (m, 2H), 2.03 (s, 3H), 1.68-1.81 (m, 4H).

### Example 125: 2-({4-[2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]-6-fluoro-1-{[(2S)-oxetan-2-yl]methyl}-1H-1,3-benzodiazole (Compound 249a)

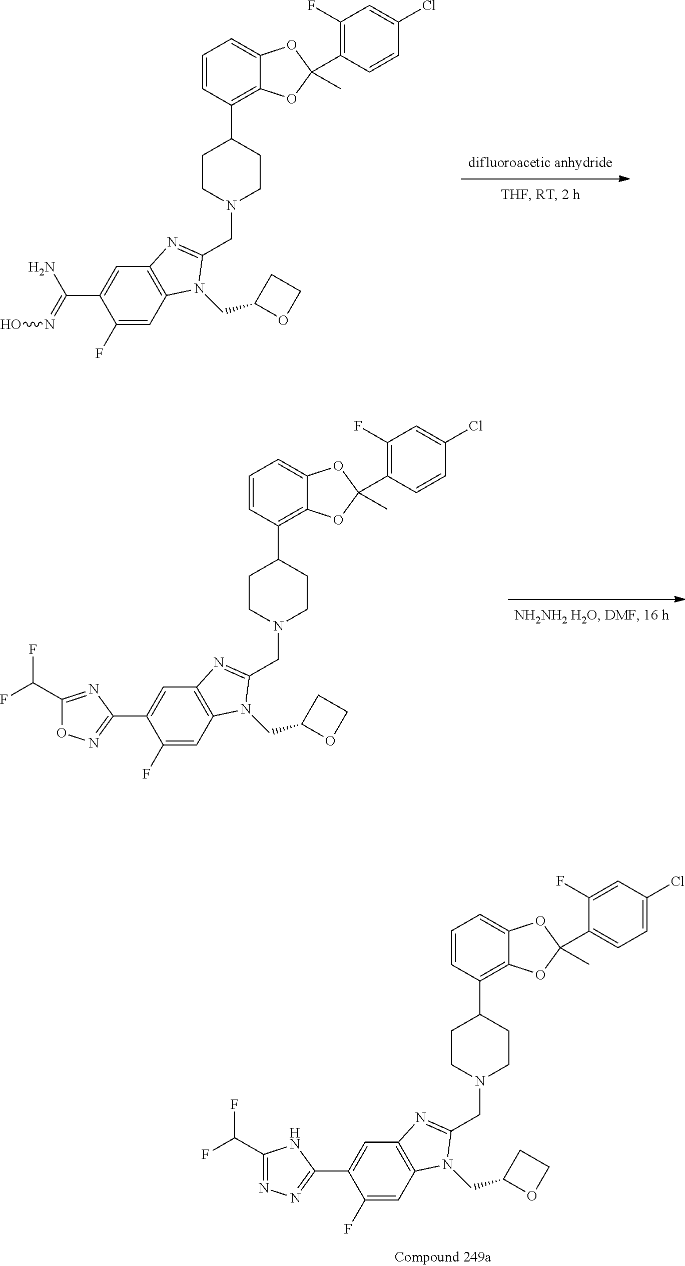 [SMILES: CC1(c2ccc(Cl)cc2F)Oc2cccc(C3CCN(Cc4nc5cc(C(N)=NO)c(F)cc5n4C[C@@H]4CCO4)CC3)c2O1 ; CC1(c2ccc(Cl)cc2F)Oc2cccc(C3CCN(Cc4nc5cc(-c6noc(C(F)F)n6)c(F)cc5n4C[C@@H]4CCO4)CC3)c2O1 ; CC1(c2ccc(Cl)cc2F)Oc2cccc(C3CCN(Cc4nc5cc(-c6nnc(C(F)F)[nH]6)c(F)cc5n4C[C@@H]4CCO4)CC3)c2O1]

### Step A: 3-(2-((4-(2-(4-chloro-2-fluorophenyl)-2-methylbenzo[d][1,3]dioxol-4-yl)piperidin-1-yl)methyl)-6-fluoro-1-(((S)-oxetan-2-yl)methyl)-1H-benzo[d]imidazol-5-yl)-5-(difluoromethyl)-1,2,4-oxadiazole

A mixture of 2-((4-(2-(4-chloro-2-fluorophenyl)-2-methylbenzo[d][1,3]dioxol-4-yl)piperidin-1-yl)methyl)-6-fluoro-N′-hydroxy-1-(((S)-oxetan-2-yl)methyl)-1H-benzo[d]imidazole-5-carboximidamide (90 mg, 0.14 mmol) and difluoroacetic anhydride (74 mg, 0.43 mmol) in THE (3 ml) was stirred at RT for 2 hours. After the reaction was completed, the mixture was diluted with EA (20 ml), washed with H₂O (10 ml×3), adjusted to pH=7 with sodium bicarbonate solution. The organic layer was dried over anhydrous sodium sulfate, filtered and evaporated to dryness and give 3-(2-((4-(2-(4-chloro-2-fluorophenyl)-2-methylbenzo[d][1,3]dioxol-4-yl)piperidin-1-yl)methyl)-6-fluoro-1-(((S)-oxetan-2-yl)methyl)-1H-benzo[d]imidazol-5-yl)-5-(difluoromethyl)-1,2,4-oxadiazole (50 mg, yield: 54%).

MS Calcd.: 683.2; MS Found: 684.2 [M+H]⁺.

### Step B: 2-({4-[2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]-6-fluoro-1-{[(2S)-oxetan-2-yl]methyl}-1H-1,3-benzodiazole (Compound 249a)

A mixture of 3-(2-((4-(2-(4-chloro-2-fluorophenyl)-2-methylbenzo[d][1,3]dioxol-4-yl)piperidin-1-yl)methyl)-6-fluoro-1-(((S)-oxetan-2-yl)methyl)-1H-benzo[d]imidazol-5-yl)-5-(difluoromethyl)-1,2,4-oxadiazole (50 mg) and hydrazine hydrate (39 mg, 0.78 mmol) in DMF (2 ml) was stirred at RT for 16 hours. After the reaction was completed, the reaction mixture was purified by prep-HPLC directly to give 2-({4-[2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]-6-fluoro-1-{[(2S)-oxetan-2-yl]methyl}-1H-1,3-benzodiazole (1.04 mg, yield: 1%) as white solid.

MS Calcd.: 682.2; MS Found: 683.3 [M+H]⁺.

¹H NMR (400 MHz, DMSO-d₆): δ 14.73 (s, 1H), 8.27 (d, J=6.0 Hz, 1H), 7.97 (d, J=10.8 Hz, 1H), 7.56-7.63 (m, 2H)), 7.35 (dd, J=8.4 Hz, J=1.6 Hz, 1H), 7.16 (t, J=53.6 Hz, 2H), 6.80-6.86 (m, 1H), 6.73-6.79 (m, 1H), 5.02-5.10 (m, 1H), 4.73-4.85 (m, 3H), 4.57-4.65 (m, 1H), 4.47-4.55 (m, 1H), 4.36-4.43 (m, 1H), 3.67-3.79 (m, 2H), 3.22-3.32 (m, 1H), 2.89-3.05 (m, 1H), 2.65-2.78 (m, 1H), 2.30-2.40 (m, 1H), 1.96-2.14 (m, 8H)

### Example 126: 2-({4-[2-(4-chloro-2-fluorophenyl)-2-methyl-2H-1,3-benzodioxol-4-yl]piperidin-1-yl}methyl)-5-[1-(2-methanesulfonylethyl)-1H-1,2,3-triazol-4-yl]-1-{[(2S)-oxetan-2-yl]methyl}-1H-1,3-benzodiazole (Compound 250a)

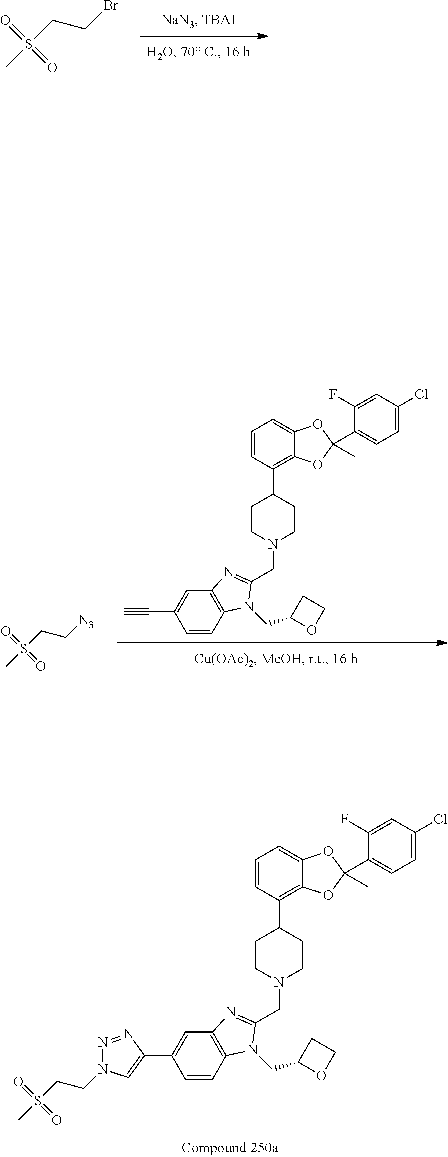

### Step A: 1-azido-2-(methylsulfonyl)ethane

To a solution of 1-bromo-2-(methylsulfonyl)ethane (400 mg, 2.15 mmol) in H₂O (5 mL) was added NaN₃ (419 mg, 6.45 mmol) and TBAI (80 mg, 0.215 mmol).

The reaction was stirred at 70° C. for 16 hours. After the reaction was completed, the reaction was quenched with water (20 mL) and extracted with ethyl acetate (50 mL×3). The organic layer was combined and washed with brine (50 mL×2), dried over sodium sulfate, filtered and concentrated in vacuum to give 1-azido-2-(methylsulfonyl)ethane (180 mg, 56% yield) as yellow oil.

### Step B: 2-({4-[2-(4-chloro-2-fluorophenyl)-2-methyl-2H-1,3-benzodioxol-4-yl]piperidin-1-yl}methyl)-5-[1-(2-methanesulfonylethyl)-1H-1,2,3-triazol-4-yl]-1-{[(2S)-oxetan-2-yl]methyl}-1H-1,3-benzodiazole (Compound 250a)

To a solution of 2-((4-(2-(4-chloro-2-fluorophenyl)-2-methylbenzo[d][1,3]dioxol-4-yl)piperidin-1-yl)methyl)-5-ethynyl-1-(((S)-oxetan-2-yl)methyl)-1H-benzo[d]imidazole (100 mg) in MeOH (1 mL) was added 1-azido-2-(methylsulfonyl)ethane (26 mg, 0.17 mmol) and Cu(OAc)₂·H₂O (3.5 mg, 0.017 mmol). The reaction was stirred at room temperature for 16 hours. After the reaction was completed, the reaction was filtered and the filtrate was purified by Prep-HPLC to give 2-({4-[2-(4-chloro-2-fluorophenyl)-2-methyl-2H-1,3-benzodioxol-4-yl]piperidin-1-yl}methyl)-5-[1-(2-methanesulfonylethyl)-1H-1,2,3-triazol-4-yl]-1-{[(2S)-oxetan-2-yl]methyl}-1H-1,3-benzodiazole (3.78 mg, 3% yield) as white solid.

MS Calcd.: 720.2; MS Found: 721.2 [M+H]⁺.

¹H NMR (400 MHz, DMSO-d₆): 8.67 (s, 1H), 8.17 (s, 1H), 7.83 (s, 2H), 7.54-7.64 (m, 2H), 7.35 (dd, J=8.4, J=2.0 Hz, 1H), 6.85-6.88 (m, 2H), 6.73-6.80 (m, 1H), 5.03-5.10 (m, 1H), 4.87 (t, J=6.8 Hz, 2H), 4.73-4.79 (m, 2H), 4.57-4.64 (m, 1H), 4.48-4.53 (m, 1H), 4.30-4.39 (m, 1H), 3.88 (t, J=6.8 Hz, 2H), 3.70-3.81 (m, 2H), 3.24-3.32 (m, 2H), 3.03 (s, 3H), 2.95-3.04 (m, 1H), 2.65-2.76 (m, 1H), 2.30-2.39 (m, 1H), 1.95-2.16 (m, 8H).

### Example 127: 3-{4-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-1H-1,2,3-triazol-1-yl}propanoic acid (Compound 251a)

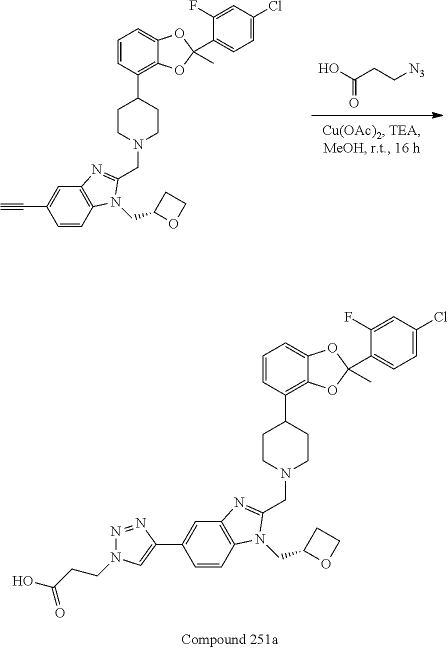 [SMILES: C#Cc1ccc2c(c1)nc(CN1CCC(c3cccc4c3OC(C)(c3ccc(Cl)cc3F)O4)CC1)n2C[C@@H]1CCO1 ; [N-]=[N+]=NCCC(=O)O ; CC1(c2ccc(Cl)cc2F)Oc2cccc(C3CCN(Cc4nc5cc(-c6cn(CCC(=O)O)nn6)ccc5n4C[C@@H]4CCO4)CC3)c2O1]

To a solution of 2-((4-(2-(4-chloro-2-fluorophenyl)-2-methylbenzo[d][1,3]dioxol-4-yl)piperidin-1-yl)methyl)-5-ethynyl-1-(((S)-oxetan-2-yl)methyl)-1H-benzo[d]imidazole (50 mg, 0.08 mmol) in MeOH (1 mL) was added 3-azidopropanoic acid (10 mg, 0.08 mmol), Cu(OAc)₂·H₂O (1.7 mg, 0.008 mmol) and TEA (26.5 mg, 0.24 mmol). The reaction mixture was stirred at room temperature for 16 hours. After the reaction was completed, the reaction was filtered and purified by Prep-HPLC to give 3-{4-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-1H-1,2,3-triazol-1-yl}propanoic acid (11.94 mg, 19% yield) as white solid.

MS Calcd.: 686.2; MS Found: 687.4[M+H]⁺.

¹H NMR (400 MHz, DMSO-d₆): 8.61 (s, 1H), 8.17 (s, 1H), 7.80-7.88 (m, 2H), 7.55-7.64 (m, 2H), 7.35 (dd, J=8.4 Hz, J=2.0 Hz, 1H), 6.84-6.89 (m, 2H), 6.73-6.80 (m, 1H), 5.02-5.09 (m, 1H), 4.71-4.86 (m, 3H), 4.58-4.64 (m, 3H), 4.48-4.53 (m, 1H), 4.33-4.40 (m, 1H), 3.72-3.80 (m, 2H), 3.30-3.40 (m, 2H), 2.95-3.05 (m, 3H), 2.68-2.76 (m, 1H), 2.31-2.42 (m, 1H), 1.95-2.20 (m, 7H).

### Example 128: 2-({4-[2-(4-chloro-2-fluorophenyl)-2-methyl-2H-1,3-benzodioxol-4-yl]piperidin-1-yl}methyl)-5-[5-(methylsulfanyl)-4H-1,2,4-triazol-3-yl]-1-{[(2S)-oxetan-2-yl]methyl}-1H-1,3-benzodiazole (Compound 252a)

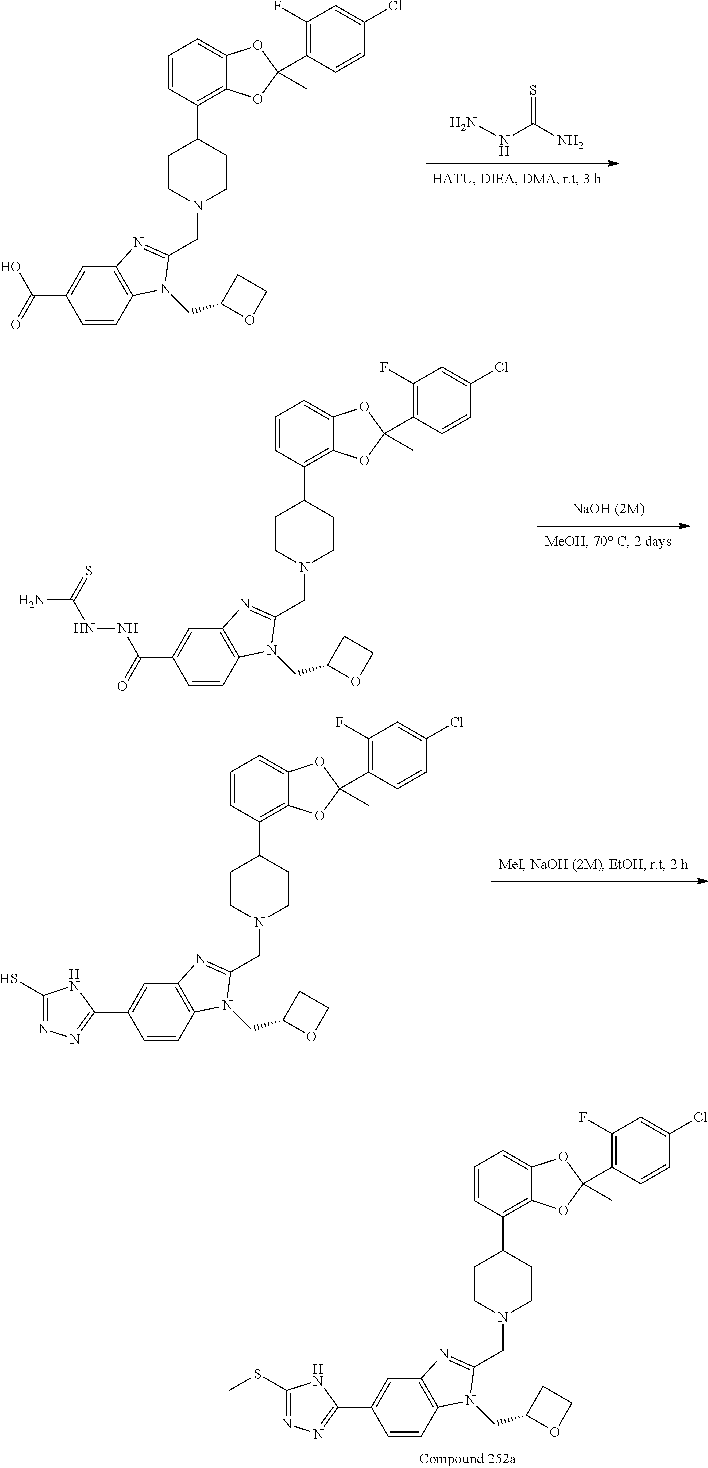

### Step A: 2-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonyl)hydrazine-1-carbothioamide

To a solution of 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carboxylic acid (230 mg, 0.39 mmol) in DMA (5 mL) was added hydrazinecarbothioamide (91 mg, 0.78 mmol), DIEA (151 mg, 1.17 mmol) and HATU (193 mg, 0.51 mmol). The reaction was stirred at rt for 3 h. After the reaction was completed, the reaction was quenched with water (15 mL) and extracted with ethyl acetate (20 mL×2). The organic layers were combined and washed with brine (20 mL), dried over sodium sulfate, filtered and concentrated in vacuum. The residue was purified by silica gel column (DCM/MeOH=30/1) to give 2-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonyl)hydrazine-1-carbothioamide (240 mg, 93% yield) as white solid.

MS Calcd.: 664.2; MS Found: 665.3 [M+H]⁺.

### Step B: 5-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-4H-1,2,4-triazole-3-thiol

To a solution of 2-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonyl)hydrazine-1-carbothioamide (240 mg, 0.36 mmol) in MeOH (6.0 mL) was added 2 M NaOH (2.0 mL) and the reaction was stirred at 70° C. for 2 days. After the reaction was completed, the reaction was quenched with water (10 mL) and extracted with DCM/MeOH (20:1, 20 mL×2). The organic layers were combined and dried over sodium sulfate, filtered and concentrated in vacuum. The residue was purified by silica gel column (DCM/MeOH=20/1) to give 5-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-4H-1,2,4-triazole-3-thiol (180 mg, 77% yield) as white solid.

MS Calcd.: 646.2; MS Found: 647.3 [M+H]⁺.

### Step C: 2-({4-[2-(4-chloro-2-fluorophenyl)-2-methyl-2H-1,3-benzodioxol-4-yl]piperidin-1-yl}methyl)-5-[5-(methylsulfanyl)-4H-1,2,4-triazol-3-yl]-1-{[(2S)-oxetan-2-yl]methyl}-1H-1,3-benzodiazole (Compound 252a)

To a solution of 5-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-4H-1,2,4-triazole-3-thiol (130 mg, 0.20 mmol) in 2 M NaOH (1.5 mL) was added MeI (28.6 mg, 0.20 mmol) in EtOH (1.5 mL) and the reaction was stirred at rt for 2 hours. After the reaction was completed, the reaction mixture was purified by Prep-HPLC to give 2-({4-[2-(4-chloro-2-fluorophenyl)-2-methyl-2H-1,3-benzodioxol-4-yl]piperidin-1-yl}methyl)-5-[5-(methylsulfanyl)-4H-1,2,4-triazol-3-yl]-1-{[(2S)-oxetan-2-yl]methyl}-1H-1,3-benzodiazole (27.21 mg, yield: 20%) as a white solid.

MS Calcd.: 660.2; MS Found: 661.2 [M+H]⁺.

¹H NMR (400 MHz, DMSO-d₆): δ 8.17 (d, J=1.2 Hz, 1H), 7.84-7.87 (m, 1H), 7.75 (d, J=8.4 Hz, 1H), 7.52-7.60 (m, 2H), 7.33 (dd, J=8.4 Hz, 1.6 Hz, 1H), 6.72-6.81 (m, 3H), 5.10-5.18 (m, 1H), 4.72-4.80 (m, 1H), 4.58-4.65 (m, 1H), 4.38-4.51 (m, 2H), 3.95 (d, J=13.6 Hz, 1H), 3.78 (d, J=13.2 Hz, 1H), 2.98-3.03 (m, 1H), 2.85-2.92 (m, 1H), 2.63-2.73 (m, 2H), 2.61 (s, 3H), 2.41-2.51 (m, 1H), 2.12-2.30 (m, 2H), 2.02 (s, 3H), 1.68-1.81 (m, 4H).

### Example 129: 2-({4-[2-(4-chloro-2-fluorophenyl)-2-methyl-2H-1,3-benzodioxol-4-yl]piperidin-1-yl}methyl)-5-(5-methanesulfonyl-4H-1,2,4-triazol-3-yl)-1-{[(2S)-oxetan-2-yl]methyl}-1H-1,3-benzodiazole (Compound 253a)

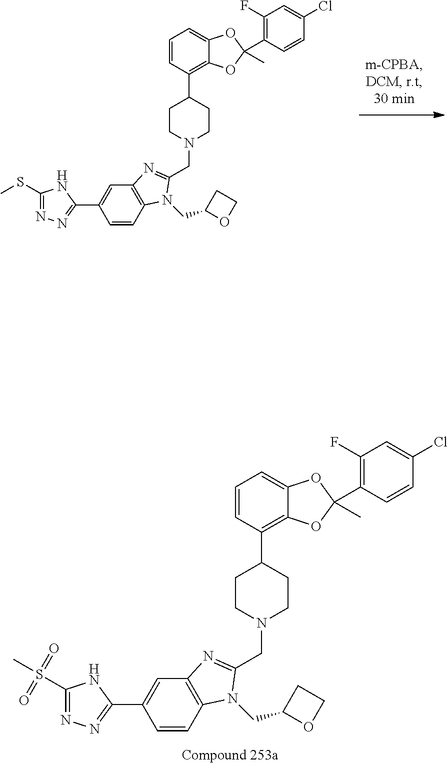 [SMILES: CSc1nnc(-c2ccc3c(c2)nc(CN2CCC(c4cccc5c4OC(C)(c4ccc(Cl)cc4F)O5)CC2)n3C[C@@H]2CCO2)[nH]1 ; CC1(c2ccc(Cl)cc2F)Oc2cccc(C3CCN(Cc4nc5cc(-c6nnc(S(C)(=O)=O)[nH]6)ccc5n4C[C@@H]4CCO4)CC3)c2O1]

To a solution of 2-((4-(2-(4-chloro-2-fluorophenyl)-2-methylbenzo[d][1,3]dioxol-4-yl)piperidin-1-yl)methyl)-5-(5-(methylthio)-4H-1,2,4-triazol-3-yl)-1-(((S)-oxetan-2-yl)methyl)-11H-benzo[d]imidazole (23 mg, 0.035 mmol) in DCM (2.0 mL) was added m-CPBA (7.8 mg, 0.045 mmol) and the reaction was stirred at rt for 30 min. After the reaction was completed, the reaction mixture was quenched with water (10 mL) and extracted with DCM/MeOH (20:1, 20 mL×2). The organic layers were combined and dried over sodium sulfate, filtered and concentrated in vacuum. The residue was purified by Prep-HPLC to give 2-({4-[2-(4-chloro-2-fluorophenyl)-2-methyl-2H-1,3-benzodioxol-4-yl]piperidin-1-yl}methyl)-5-(5-methanesulfonyl-4H-1,2,4-triazol-3-yl)-1-{[(2S)-oxetan-2-yl]methyl}-1H-1,3-benzodiazole (10.10 mg, yield: 42%) as a white solid.

MS Calcd.: 692.2; MS Found: 693.2 [M+H]⁺.

¹H NMR (400 MHz, DMSO-d6): δ 8.35 (s, 1H), 7.96 (d, J=8.8 Hz, 1H), 7.76 (d, J=8.4 Hz, 1H), 7.51-7.58 (m, 2H), 7.30 (d, J=8.4 Hz, 1H), 6.75-6.80 (m, 2H), 6.68-6.71 (m, 1H), 5.04-5.15 (m, 2H), 4.88-4.99 (m, 3H), 4.44-4.51 (m, 1H), 4.31-4.38 (m, 1H), 3.64-3.76 (m, 2H), 3.35-3.47 (m, 2H), 3.00 (s, 3H), 2.83-2.92 (m, 1H), 2.62-2.72 (m, 1H), 2.27-2.46 (m, 3H), 2.01 (s, 3H), 1.63-1.74 (m, 2H).

### Example 130: 1-{[2-({4-[2-(4-chloro-2-fluorophenyl)-2-methyl-2H-1,3-benzodioxol-4-yl]piperidin-1-yl}methyl)-6-[5-(trifluoromethyl)-4H-1,2,4-triazol-3-yl]-3H-imidazo[4,5-b]pyridin-3-yl]methyl}cyclopropane-1-carbonitrile (Compound 254)

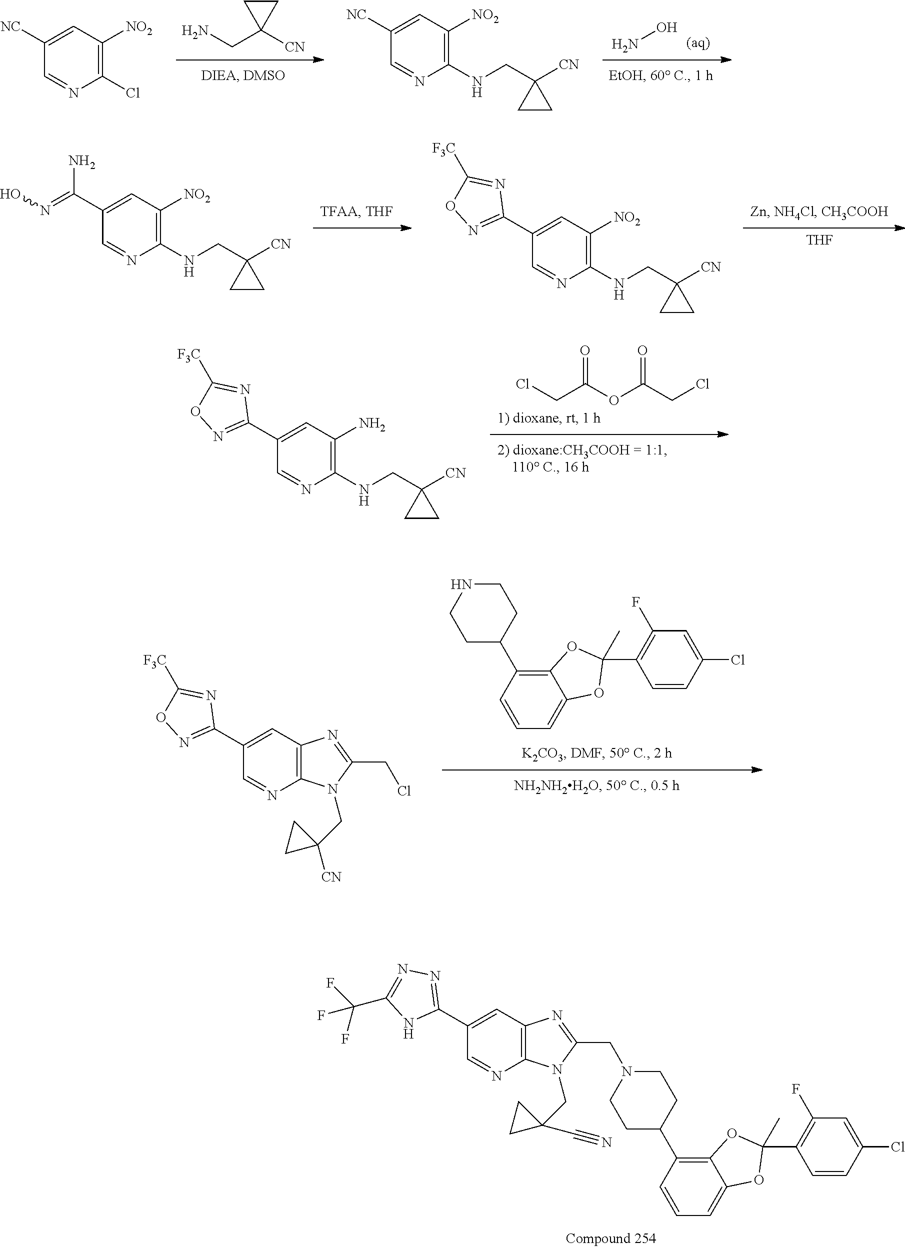

### Step A: 6-(((1-cyanocyclopropyl)methyl)amino)-5-nitronicotinonitrile

To a solution of 6-chloro-5-nitronicotinonitrile (300 mg, 1.6 mmol) and 1-(aminomethyl)cyclopropane-1-carbonitrile HCl salt (240 mg, 1.8 mmol) in DMSO (5 mL) was added DIEA (633 mg, 4.8 mmol) at room temperature. The reaction was stirred at 70° C. for 2 hours. After the reaction was completed, the reaction was quenched with water (50 mL) and extracted with ethyl acetate (100 mL×3). The organic layer was combined and washed with brine (100 mL×2), dried over sodium sulfate, filtered and concentrated in vacuum. The residue was purified by column chromatography (PE:EA=3:1) to give 6-(((1-cyanocyclopropyl)methyl)amino)-5-nitronicotinonitrile (400 mg, 100% yield) as an orange solid.

MS Calcd.: 243.1; MS Found: 244.1 [M+H]⁺.

### Step B: 6-(((1-cyanocyclopropyl)methyl)amino)-N′-hydroxy-5-nitronicotinimidamide

To a solution of 6-(((1-cyanocyclopropyl)methyl)amino)-5-nitronicotinonitrile (200 mg, 0.8 mmol) in EtOH (9 mL) was added hydroxylamine (108.6 mg, 1.6 mmol, 50% in water). The reaction was stirred at 60° C. for 1 hour. After the reaction was completed, the reaction was quenched with water (30 mL) and extracted with ethyl acetate (50 mL×3). The organic layer was combined and washed with brine (50 mL×2), dried over sodium sulfate, filtered and concentrated in vacuum. The residue was purified by column chromatography (DCM:MeOH=10:1) to give 6-(((1-cyanocyclopropyl)methyl)amino)-N′-hydroxy-5-nitronicotinimidamide (240 mg, crude) as yellow solid.

MS Calcd.: 276.1; MS Found: 277.1 [M+H]⁺.

### Step C: 1-(((3-nitro-5-(5-(trifluoromethyl)-1,2,4-oxadiazol-3-yl)pyridin-2-yl)amino)methyl) cyclopropane-1-carbonitrile

To a solution of 6-(((1-cyanocyclopropyl)methyl)amino)-N′-hydroxy-5-nitronicotinimidamide (240 mg, 0.87 mmol) in THE (3 mL) was added TFAA (730 mg, 3.48 mmol) at room temperature. The reaction was stirred at room temperature for 1 hour. After the reaction was completed, the reaction was quenched with NaHCO₃ aqueous solution (50 mL) and extracted with ethyl acetate (100 mL×3). The organic layer was combined and washed with brine (50 mL×2), dried over sodium sulfate, filtered and concentrated in vacuum. The residue was purified by column chromatography (PE:EA=5:1) to give 1-(((3-nitro-5-(5-(trifluoromethyl)-1,2,4-oxadiazol-3-yl)pyridin-2-yl)amino)methyl)cyclopropane-1-carbonitrile (240 mg, 66% yield) as yellow solid.

MS Calcd.: 354.1; MS Found: 355.1 [M+H]⁺.

### Step D: 1-(((3-amino-5-(5-(trifluoromethyl)-1,2,4-oxadiazol-3-yl)pyridin-2-yl)amino)methyl) cyclopropane-1-carbonitrile

To a solution of 1-(((3-nitro-5-(5-(trifluoromethyl)-1,2,4-oxadiazol-3-yl)pyridin-2-yl)amino)methyl) cyclopropane-1-carbonitrile (200 mg, 0.56 mmol) in THF/CH₃COOH (3 mL/1 mL) was added Zn (183.6 mg g, 2.82 mmol) and NH₄Cl (302 mg, 5.6 mmol). The reaction was stirred at 70° C. for 1 hour. After the reaction was completed, the reaction was filtered and concentrated in vacuum. The residue was purified by column chromatography (PE:EA=1:1) to give 1-(((3-amino-5-(5-(trifluoromethyl)-1,2,4-oxadiazol-3-yl)pyridin-2-yl)amino)methyl) cyclopropane-1-carbonitrile (160 mg, 87% yield) as yellow solid.

MS Calcd.: 324.1; MS Found: 325.1 [M+H]⁺.

### Step E: 1-((2-(chloromethyl)-6-(5-(trifluoromethyl)-1,2,4-oxadiazol-3-yl)-3H-imidazo[4,5-b]pyridin-3-yl)methyl)cyclopropane-1-carbonitrile

To a solution of 1-(((3-amino-5-(5-(trifluoromethyl)-1,2,4-oxadiazol-3-yl)pyridin-2-yl)amino)methyl) cyclopropane-1-carbonitrile (160 mg, 0.5 mmol) in dioxane (2 mL) was added 2-chloroacetic anhydride (100 mg, 0.6 mmol) at room temperature. The reaction was stirred at room temperature for 1 hour. Then CH₃COOH (2 mL) was added and the reaction was stirred at 110° C. for 16 hours. After the reaction was completed, the reaction was concentrated in vacuum. The residue was purified by column chromatography (PE:EA=2:1) to give 1-((2-(chloromethyl)-6-(5-(trifluoromethyl)-1,2,4-oxadiazol-3-yl)-3H-imidazo[4,5-b]pyridin-3-yl)methyl) cyclopropane-1-carbonitrile (150 mg, 80% yield) as yellow solid.

MS Calcd.: 382.1; MS Found: 383.0 [M+H]⁺.

### Step F: 1-{[2-({4-[2-(4-chloro-2-fluorophenyl)-2-methyl-2H-1,3-benzodioxol-4-yl]piperidin-1-yl}methyl)-6-[5-(trifluoromethyl)-4H-1,2,4-triazol-3-yl]-3H-imidazo[4,5-b]pyridin-3-yl]methyl}cyclopropane-1-carbonitrile (Compound 254)

A mixture of 1-((2-(chloromethyl)-6-(5-(trifluoromethyl)-1,2,4-oxadiazol-3-yl)-3H-imidazo[4,5-b]pyridin-3-yl)methyl)cyclopropane-1-carbonitrile (50 mg, 0.13 mmol), 4-(2-(4-chloro-2-fluorophenyl)-2-methylbenzo[d][1,3]dioxol-4-yl)piperidine (60 mg) and K₂CO₃ (54 mg, 0.39 mmol) in DMF (1 mL) was stirred at 50° C. for 2 hours. Then NH₂NH₂·H₂O (32.7 mg, 0.65 mmol) was added into the reaction. The reaction was stirred at 50° C. for 2 hours. After the reaction was completed, the reaction was filtered and purified by Prep-HPLC to give 1-{[2-({4-[2-(4-chloro-2-fluorophenyl)-2-methyl-2H-1,3-benzodioxol-4-yl]piperidin-1-yl}methyl)-6-[5-(trifluoromethyl)-4H-1,2,4-triazol-3-yl]-3H-imidazo[4,5-b]pyridin-3-yl]methyl}cyclopropane-1-carbonitrile (2.03 mg, 2% yield) as a white solid.

MS Calcd.: 692.2; MS Found: 693.4 [M+H]⁺.

¹H NMR (400 MHz, DMSO-d₆): 15.44 (brs, 1H), 9.02 (d, J=2.0 Hz, 1H), 8.62 (d, J=2.0 Hz, 1H), 7.52-7.59 (m, 2H), 7.33 (dd, J=8.4 Hz, J=2.0 Hz, 1H), 6.74-6.82 (m, 3H), 4.76 (s, 2H), 4.03 (s, 2H), 2.97-3.02 (m, 2H), 2.65-2.72 (m, 1H), 2.24-2.33 (m, 2H), 2.02 (s, 3H), 1.70-1.86 (m, 4H), 1.51-1.55 (m, 2H), 1.37-1.42 (m, 2H).

### Example 131: 2-({4-[2-(4-chlorophenyl)-2-methyl-2H-1,3-benzodioxol-4-yl]-1,2,3,6-tetrahydropyridin-1-yl}methyl)-1-{[(2S)-oxetan-2-yl]methyl}-5-[5-(trifluoromethyl)-4H-1,2,4-triazol-3-yl]-1H-1,3-benzodiazole (Compound 255a)

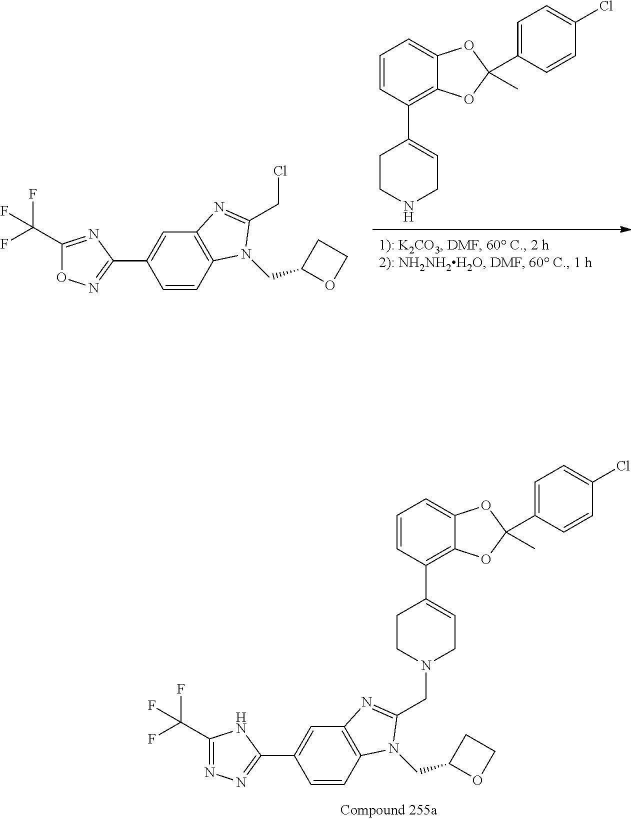 [SMILES: FC(F)(F)c1nc(-c2ccc3c(c2)nc(CCl)n3C[C@@H]2CCO2)no1 ; CC1(c2ccc(Cl)cc2)Oc2cccc(C3=CCNCC3)c2O1 ; CC1(c2ccc(Cl)cc2)Oc2cccc(C3=CCN(Cc4nc5cc(-c6nnc(C(F)(F)F)[nH]6)ccc5n4C[C@@H]4CCO4)CC3)c2O1]

A mixture of (S)-3-(2-(chloromethyl)-1-(oxetan-2-ylmethyl)-1H-benzo[d]imidazol-5-yl)-5-(trifluoromethyl)-1,2,4-oxadiazole (72 mg, 0.19 mmol), 4-(2-(4-chlorophenyl)-2-methylbenzo[d][1,3]dioxol-4-yl)-1,2,3,6-tetrahydropyridine (70 mg) and K₂CO₃ (80 mg, 0.57 mmol) in DMF (2 mL) was stirred at 60° C. for 2 hours. Then NH₂NH₂·H₂O (48 mg, 0.95 mmol) was added into the reaction mixture. The mixture was stirred at 60° C. for an additional 1 hour. After the reaction was completed, the reaction was filtered and the filtrate was purified by Prep-HPLC to give 2-({4-[2-(4-chlorophenyl)-2-methyl-2H-1,3-benzodioxol-4-yl]-1,2,3,6-tetrahydropyridin-1-yl}methyl)-1-{[(2S)-oxetan-2-yl]methyl}-5-[5-(trifluoromethyl)-4H-1,2,4-triazol-3-yl]-1H-1,3-benzodiazole (12.57 mg, 10% yield) as a yellow solid.

MS Calcd.: 662.2; MS Found: 663.4 [M+H]⁺.

¹H NMR (400 MHz, DMSO-d₆): 8.43 (d, J=1.2 Hz, 1H), 8.04 (dd, J=8.8 Hz, J=1.6 Hz, 1H), 7.83 (d, J=8.4 Hz, 1H), 7.57-7.60 (m, 2H), 7.38-7.41 (m, 2H), 6.81-6.92 (m, 3H), 6.44-6.48 (m, 1H), 5.18-5.24 (m, 1H), 4.90 (s, 2H), 4.73-4.81 (m, 1H), 4.60-4.69 (m, 2H), 4.36-4.45 (m, 1H), 4.18 (s, 2H), 3.68-3.80 (m, 2H), 2.95-3.03 (m, 2H), 2.75-2.83 (m, 1H), 2.45-2.54 (m, 1H), 1.99 (s, 3H).

### Example 132: 2-({4-[2-(4-chloro-2-fluorophenyl)-2-methyl-2H-1,3-benzodioxol-4-yl]-1,2,3,6-tetrahydropyridin-1-yl}methyl)-1-{[(2S)-oxetan-2-yl]methyl}-5-[5-(trifluoromethyl)-4H-1,2,4-triazol-3-yl]-1H-1,3-benzodiazole (Compound 256a)

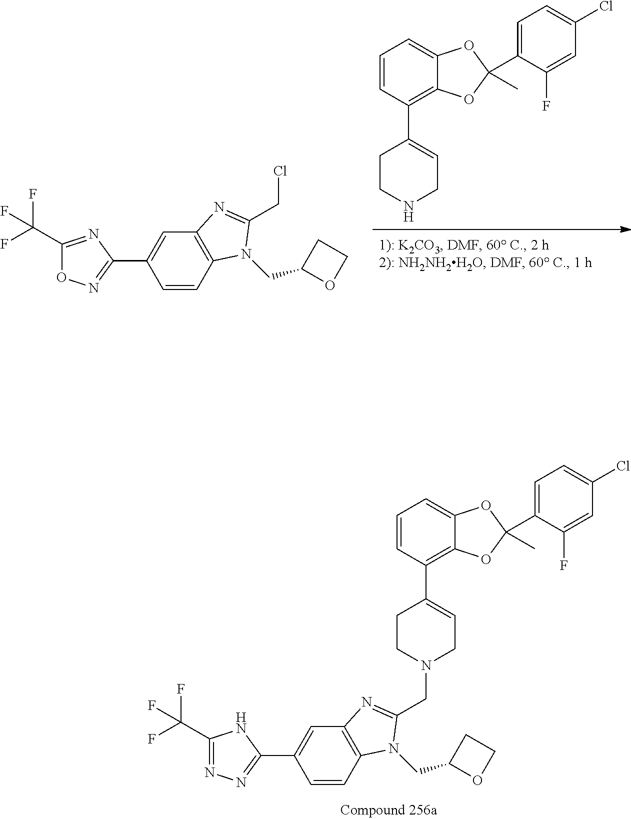 [SMILES: FC(F)(F)c1nc(-c2ccc3c(c2)nc(CCl)n3C[C@@H]2CCO2)no1 ; CC1(c2ccc(Cl)cc2F)Oc2cccc(C3=CCNCC3)c2O1 ; CC1(c2ccc(Cl)cc2F)Oc2cccc(C3=CCN(Cc4nc5cc(-c6nnc(C(F)(F)F)[nH]6)ccc5n4C[C@@H]4CCO4)CC3)c2O1]

A mixture of (S)-3-(2-(chloromethyl)-1-(oxetan-2-ylmethyl)-1H-benzo[d]imidazol-5-yl)-5-(trifluoromethyl)-1,2,4-oxadiazole (100 mg, 0.27 mmol), 4-(2-(4-chloro-2-fluorophenyl)-2-methylbenzo[d][1,3]dioxol-4-yl)-1,2,3,6-tetrahydropyridine (113 mg) and K₂CO₃ (111 mg, 0.80 mmol) in DMF (2 mL) was stirred at 60° C. for 2 hours. Then NH₂NH₂·H₂O (48 mg, 0.95 mmol) was added into the reaction. The reaction was stirred at 60° C. for 1 hour. After the reaction was completed, the reaction was filtered and purified by Prep-HPLC to give 2-({4-[2-(4-chloro-2-fluorophenyl)-2-methyl-2H-1,3-benzodioxol-4-yl]-1,2,3,6-tetrahydropyridin-1-yl}methyl)-1-{[(2S)-oxetan-2-yl]methyl}-5-[5-(trifluoromethyl)-4H-1,2,4-triazol-3-yl]-1H-1,3-benzodiazole (44.09 mg, 24% yield) as a yellow solid.

MS Calcd.: 680.2; MS Found: 681.4 [M+H]⁺.

¹H NMR (400 MHz, DMSO-d₆): 8.28 (s, 1H), 7.93 (dd, J=8.4 Hz, J=1.2 Hz, 1H), 7.81 (d, J=8.8 Hz, 1H), 7.52-7.59 (m, 2H), 7.33 (dd, J=8.4 Hz, J=2.0 Hz, 1H), 6.81-6.88 (m, 3H), 6.37-6.40 (m, 1H), 5.07-5.15 (m, 1H), 4.73-4.81 (m, 1H), 4.60-4.65 (m, 1H), 4.44-4.50 (m, 1H), 4.35-4.44 (m, 1H), 4.06 (dd, J=13.2 Hz, J=6.4 Hz, 1H), 3.92 (dd, J=13.2 Hz, J=5.6 Hz, 1H), 3.16-3.24 (m, 2H), 2.72-2.80 (m, 2H), 2.60-2.72 (m, 1H), 2.38-2.55 (m, 3H), 2.03 (s, 3H).

### Example 133: 2-({4-[2-(5-chloropyridin-2-yl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 257a)

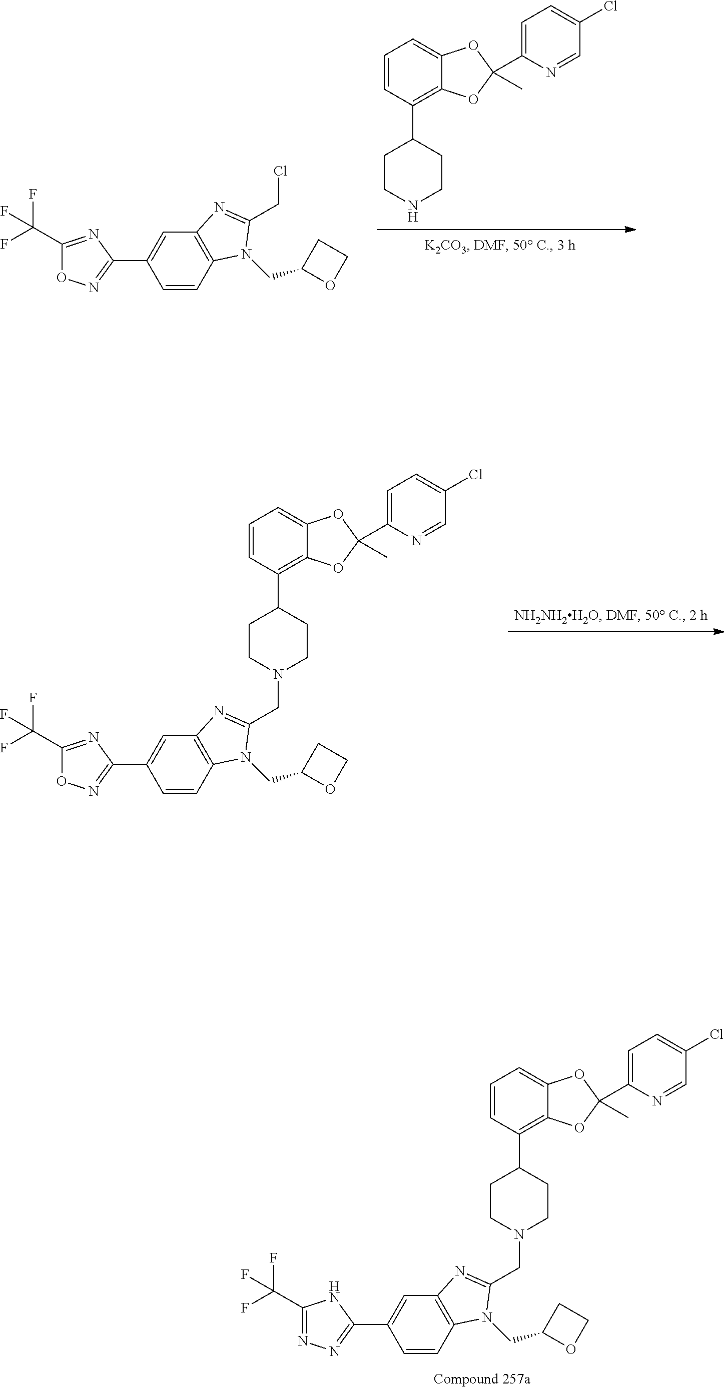 [SMILES: FC(F)(F)c1nc(-c2ccc3c(c2)nc(CCl)n3C[C@@H]2CCO2)no1 ; CC1(c2ccc(Cl)cn2)Oc2cccc(C3CCNCC3)c2O1 ; CC1(c2ccc(Cl)cn2)Oc2cccc(C3CCN(Cc4nc5cc(-c6noc(C(F)(F)F)n6)ccc5n4C[C@@H]4CCO4)CC3)c2O1 ; CC1(c2ccc(Cl)cn2)Oc2cccc(C3CCN(Cc4nc5cc(-c6nnc(C(F)(F)F)[nH]6)ccc5n4C[C@@H]4CCO4)CC3)c2O1]

### Step A: 3-(2-((4-(2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazol-5-yl)-5-(trifluoromethyl)-1,2,4-oxadiazole

A mixture of (S)-3-(2-(chloromethyl)-1-(oxetan-2-ylmethyl)-1H-benzo[d]imidazol-5-yl)-5-(trifluoromethyl)-1,2,4-oxadiazole (40 mg, 0.1 mmol), 5-chloro-2-(2-methyl-4-(piperidin-4-yl)benzo[d][1,3]dioxol-2-yl)pyridine (42 mg) and K₂CO₃ (42 mg, 0.3 mmol) in DMF (2 mL) was stirred at 50° C. for 3 hours. After the reaction was completed, the reaction was quenched with water (30 mL) and extracted with ethyl acetate (50 mL×3). The organic layer was combined and washed with brine (50 mL×2), dried over sodium sulfate, filtered and concentrated in vacuum to give 3-(2-((4-(2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazol-5-yl)-5-(trifluoromethyl)-1,2,4-oxadiazole (60 mg, 84% yield) as a yellow solid.

MS Calcd.: 666.2; MS Found: 667.3 [M+H]⁺.

### Step B: 2-({4-[2-(5-chloropyridin-2-yl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 257a)

To a solution of 3-(2-((4-(2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazol-5-yl)-5-(trifluoromethyl)-1,2,4-oxadiazole (60 mg, 0.09 mmol) in DMF (2 mL) was added NH₂NH₂·H₂O (0.5 mL). The reaction was stirred at 50° C. for 2 hours. After the reaction was completed, the reaction was filtered and purified by Prep-HPLC to give 2-({4-[2-(5-chloropyridin-2-yl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (21 mg, 35% yield) as a white solid.

MS Calcd.: 665.2; MS Found: 666.5 [M+H]⁺.

¹H NMR (400 MHz, DMSO-d₆): δ 8.72 (t, J=2.0 Hz, 1H), 8.13 (s, 1H), 7.98-8.04 (m, 1H), 7.91 (dd, J=8.4 Hz, 1.2 Hz, 1H), 7.61 (dd, J=8.4 Hz, 1.6 Hz, 1H), 7.56 (d, J=8.8 Hz, 1H), 6.74-6.82 (m, 3H), 5.09-5.16 (m, 1H), 4.65-4.71 (m, 1H), 4.53-4.60 (m, 1H), 4.35-4.50 (m, 2H), 3.90 (dd, J=13.2 Hz, 2.8 Hz, 1H), 3.74 (dd, J=13.6 Hz, 3.6 Hz, 1H), 2.99-3.03 (m, 1H), 2.85-2.91 (m, 1H), 2.60-2.71 (m, 2H), 2.40-2.51 (m, 1H), 2.10-2.29 (m, 2H), 2.01 (s, 3H), 1.70-1.81 (m, 4H).

### Example 134: 2-({4-[2-(4-fluorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 258a)

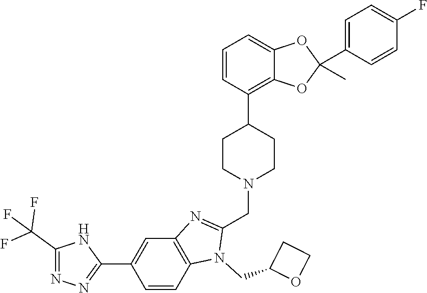 [SMILES: CC1(c2ccc(F)cc2)Oc2cccc(C3CCN(Cc4nc5cc(-c6nnc(C(F)(F)F)[nH]6)ccc5n4C[C@@H]4CCO4)CC3)c2O1]

The synthesis of Compound 258a was similar to that of Compound 257a except that 4-(2-(4-fluorophenyl)-2-methylbenzo[d][1,3]dioxol-4-yl)piperidine was used instead of 5-chloro-2-(2-methyl-4-(piperidin-4-yl)benzo[d][1,3]dioxol-2-yl)pyridine. Compound 258a: (20.22 mg, 21% yield) white solid.

MS Calcd.: 648.2; MS Found: 649.2[M+H]⁺.

¹H NMR (400 MHz, DMSO-d₆): 8.24 (s, 1H), 7.92 (dd, J=8.4 Hz, 1.2 Hz, 1H), 7.76-7.80 (m, 1H), 7.59-7.64 (m, 2H), 7.21-7.29 (m, 2H), 6.70-6.79 (m, 3H), 5.10-5.17 (m, 1H), 4.71-4.80 (m, 1H), 4.59-4.65 (m, 1H), 4.37-4.50 (m, 2H), 3.95 (d, J=13.6 Hz, 1H), 3.79 (d, J=10.0 Hz, 1H), 3.00-3.04 (m, 1H), 2.85-2.92 (m, 1H), 2.61-2.75 (m, 2H), 2.40-2.50 (m, 1H), 2.15-2.30 (m, 2H), 1.97 (s, 3H), 1.69-1.82 (m, 4H).

### Example 135: 2-({4-[2-(2,4-difluorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 259a)

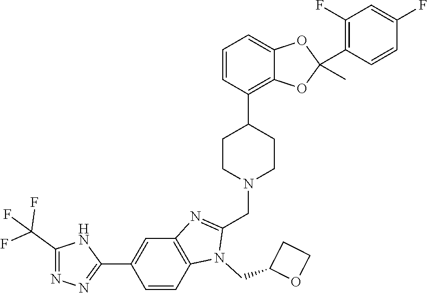 [SMILES: CC1(c2ccc(F)cc2F)Oc2cccc(C3CCN(Cc4nc5cc(-c6nnc(C(F)(F)F)[nH]6)ccc5n4C[C@@H]4CCO4)CC3)c2O1]

The synthesis of Compound 259a was similar to that of Compound 257a except that 4-(2-(2,4-difluorophenyl)-2-methylbenzo[d][1,3]dioxol-4-yl)piperidine was used instead of 5-chloro-2-(2-methyl-4-(piperidin-4-yl)benzo[d][1,3]dioxol-2-yl)pyridine. Compound 259a: (7.76 mg, 11% yield) white solid.

MS Calcd.: 666.2; MS Found: 667.2[M+H]⁺.

¹H NMR (400 MHz, DMSO-d₆): δ 8.26 (s, 1H), 7.90-7.95 (m, 1H), 7.81 (d, J=8.4 Hz, 1H), 7.57-7.64 (m, 1H), 7.32-7.40 (m, 1H), 7.09-7.14 (m, 1H), 6.72-6.80 (m, 3H), 5.10-5.19 (m, 1H), 4.73-4.82 (m, 1H), 4.60-4.67 (m, 1H), 4.37-4.51 (m, 2H), 3.95 (d, J=13.6 Hz, 1H), 3.79 (d, J=13.6 Hz, 1H), 3.00-3.04 (m, 1H), 2.85-2.90 (m, 1H), 2.60-2.75 (m, 2H), 2.40-2.50 (m, 1H), 2.14-2.33 (m, 2H), 2.02 (s, 3H), 1.68-1.86 (m, 4H).

### Example 136: 4-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)benzonitrile (Compound 260a)

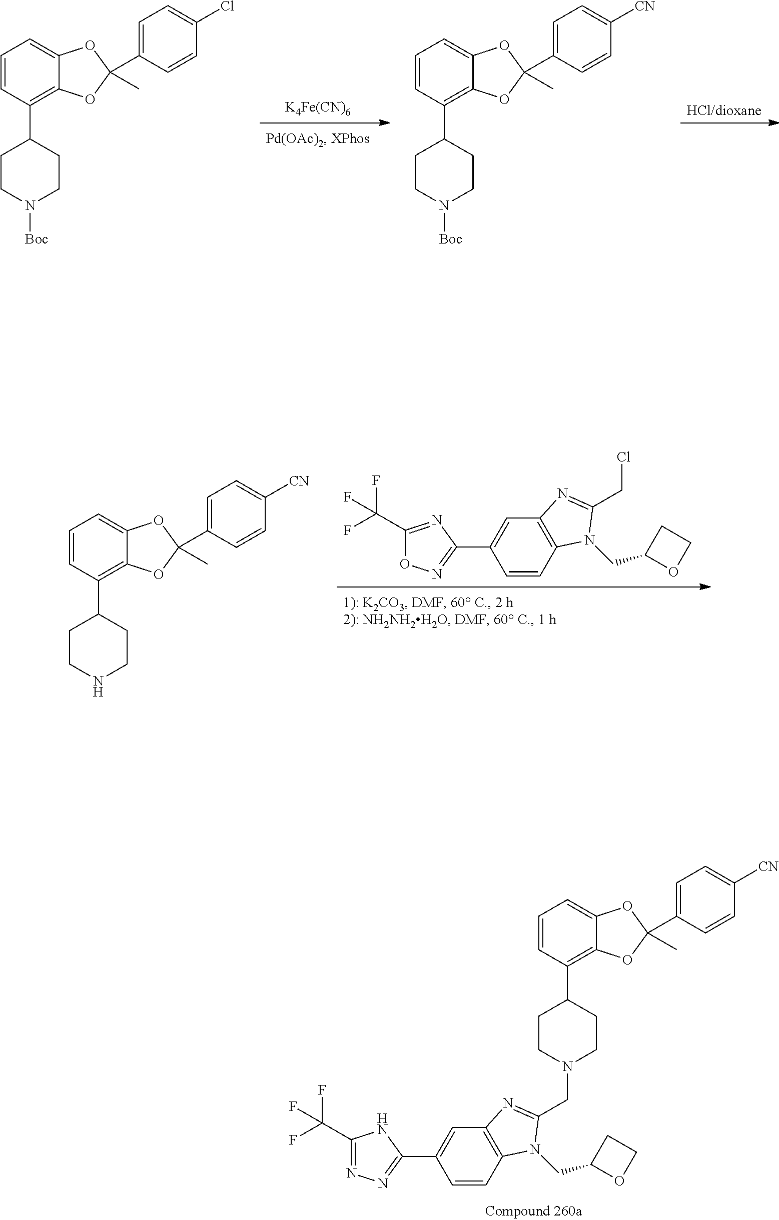

### Step A: tert-butyl 4-(2-(4-cyanophenyl)-2-methylbenzo[d][1,3]dioxol-4-yl)piperidine-1-carboxylate

A mixture of tert-butyl 4-(2-(4-chlorophenyl)-2-methylbenzo[d][1,3]dioxol-4-yl)piperidine-1-carboxylate (300 mg, 0.7 mmol), K₄Fe(CN)₆ (148 mg, 0.35 mmol), Pd(OAc)₂ (15.7 mg, 0.07 mmol), XPhos (66.6 mg, 0.14 mmol) and K₂CO₃ (24 mg, 0.18 mmol) in dioxane/H₂O (1.5 mL/1.5 mL) was degassed with Ar. The reaction was stirred at 120° C. for 10 hours under M.W. After the reaction was completed, the reaction was quenched with water (30 mL) and extracted with ethyl acetate (50 mL×3). The organic layer was combined and washed with brine (50 mL×2), dried over sodium sulfate, filtered and concentrated in vacuum. The residue was purified by column chromatography (PE:EA=5:1) to give tert-butyl 4-(2-(4-cyanophenyl)-2-methylbenzo[d][1,3]dioxol-4-yl)piperidine-1-carboxylate (300 mg, crude) as colorless oil.

MS Calcd.: 420.2; MS Found: 321.1 [M-100+H]⁺.

### Step B: 4-(2-methyl-4-(piperidin-4-yl)benzo[d][1,3]dioxol-2-yl)benzonitrile

To a solution of tert-butyl 4-(2-(4-cyanophenyl)-2-methylbenzo[d][1,3]dioxol-4-yl)piperidine-1-carboxylate (60 mg, 0.14 mmol) in dioxane (1 mL) was added HCl/dioxane (4M, 3 mL). The reaction was stirred at room temperature for 1 hour. After the reaction was completed, the reaction was concentrated in vacuum to give 4-(2-methyl-4-(piperidin-4-yl)benzo[d][1,3]dioxol-2-yl)benzonitrile (38 mg, crude) as yellow solid.

MS Calcd.: 320.2; MS Found: 321.4 [M+H]⁺.

### Step C: 4-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)benzonitrile (Compound 260a)

A mixture of (S)-3-(2-(chloromethyl)-1-(oxetan-2-ylmethyl)-1H-benzo[d]imidazol-5-yl)-5-(trifluoromethyl) 1,2,4-oxadiazole (40 mg, 0.2 mmol), 4-(2-methyl-4-(piperidin-4-yl)benzo[d][1,3]dioxol-2-yl)benzonitrile (38 mg) and K₂CO₃ (44 mg, 0.6 mmol) in DMF (1 mL) was stirred at 60° C. for 2 hours. Then NH₂NH₂·H₂O (26 mg, 1.0 mmol) was added. The reaction was stirred at 60° C. for 1 hour. After the reaction was completed, the reaction was filtered and purified by Prep-HPLC to give 4-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)benzonitrile (5.93 mg, 7% yield) as a white solid.

MS Calcd.: 655.2; MS Found: 656.4 [M+H]⁺.

¹H NMR (400 MHz, DMSO-d₆): 8.26 (s, 1H), 7.89-7.94 (m, 3H), 7.75-7.83 (m, 3H), 6.72-6.80 (m, 3H), 5.10-5.20 (m, 1H), 4.72 (dd, J=7.2 Hz, J=3.2 Hz, 1H), 4.60-4.69 (m, 1H), 4.38-4.51 (m, 2H), 3.96 (d, J=13.6 Hz, 1H), 3.80 (d, J=13.6 Hz, 1H), 3.00-3.06 (m, 1H), 2.84-2.93 (m, 1H), 2.63-2.74 (m, 2H), 2.40-2.52 (m, 1H), 2.15-2.32 (m, 2H), 2.00 (s, 3H), 1.68-1.83 (m, 4H).

### Example 137: 3-fluoro-4-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)benzonitrile (Compound 261a)

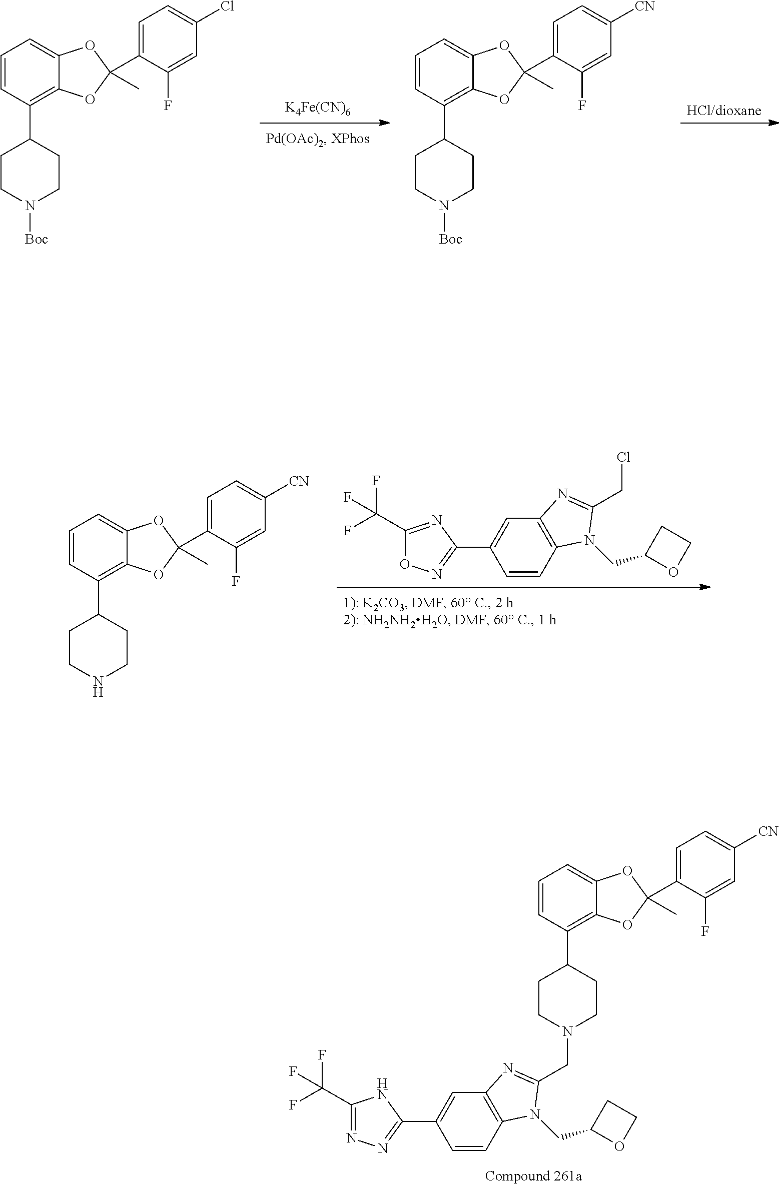

### Step A: tert-butyl 4-(2-(4-cyano-2-fluorophenyl)-2-methylbenzo[d][1,3]dioxol-4-yl)piperidine-1-carboxylate

A mixture of tert-butyl 4-(2-(4-chloro-2-fluorophenyl)-2-methylbenzo[d][1,3]dioxol-4-yl)piperidine-1-carboxylate (300 mg, 0.67 mmol), K₄Fe(CN)₆ (142 mg, 0.34 mmol) and Pd(OAc)₂ (15 mg, 0.07 mmol), XPhos (64 mg, 0.14 mmol) and K₂CO₃ (93 mg, 0.67 mmol) in dioxane/H₂O (3 mL/0.3 mL) was degassed with Ar. The reaction was stirred at 120° C. for 3 hours under M.W. After the reaction was completed, the reaction was quenched with water (30 mL) and extracted with ethyl acetate (50 mL×3). The organic layer was combined and washed with brine (50 mL×2), dried over sodium sulfate, filtered and concentrated in vacuum. The residue was purified by column chromatography (PE:EA=5:1) to give tert-butyl 4-(2-(4-cyano-2-fluorophenyl)-2-methylbenzo[d][1,3]dioxol-4-yl)piperidine-1-carboxylate (300 mg, crude) as colorless oil.

MS Calcd.: 438.2; MS Found: 383.1 [M-56+H]⁺.

### Step B: 3-fluoro-4-(2-methyl-4-(piperidin-4-yl)benzo[d][1,3]dioxol-2-yl)benzonitrile

To a solution of tert-butyl 4-(2-(4-cyano-2-fluorophenyl)-2-methylbenzo[d][1,3]dioxol-4-yl)piperidine-1-carboxylate (60 mg, 0.14 mmol) in dioxane (2 mL) was added HCl/dioxane (4M, 2 mL). The reaction was stirred at room temperature for 1 hour. After the reaction was completed, the reaction was concentrated in vacuum to give 3-fluoro-4-(2-methyl-4-(piperidin-4-yl)benzo[d][1,3]dioxol-2-yl)benzonitrile (40 mg, crude) as yellow solid.

MS Calcd.: 338.1; MS Found: 339.1 [M+H]⁺.

### Step C: 3-fluoro-4-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)benzonitrile (Compound 261a)

A mixture of (S)-3-(2-(chloromethyl)-1-(oxetan-2-ylmethyl)-1H-benzo[d]imidazol-5-yl)-5-(trifluoromethyl) 1,2,4-oxadiazole (40 mg, 0.1 mmol), 3-fluoro-4-(2-methyl-4-(piperidin-4-yl)benzo[d][1,3]dioxol-2-yl)benzonitrile (40 mg) and K₂CO₃ (45 mg, 0.3 mmol) in DMF (1 mL) was stirred at 60° C. for 2 hours. Then NH₂NH₂·H₂O (33 mg, 0.5 mmol) was added into the reaction. The reaction was stirred at 60° C. for 1 hour. After the reaction was completed, the reaction was filtered and purified by Prep-HPLC to give 3-fluoro-4-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)benzonitrile (2.25 mg, 3% yield) as a white solid.

MS Calcd.: 673.2; MS Found: 674.3 [M+H]⁺.

¹H NMR (400 MHz, DMSO-d₆): 15.19 (s, 1H), 8.27 (s, 1H), 7.98 (d, J=10.8 Hz, 1H), 7.91-7.94 (m, 1H), 7.84 (d, J=8.8 Hz, 1H), 7.71-7.76 (m, 2H), 6.73-6.83 (m, 3H), 5.10-5.19 (m, 1H), 4.75-4.82 (m, 1H), 4.60-4.68 (m, 1H), 4.37-4.50 (m, 2H), 3.92-3.98 (m, 1H), 3.75-3.85 (m, 1H), 2.90-3.07 (m, 1H), 2.85-2.95 (m, 1H), 2.65-2.75 (m, 2H), 2.40-2.50 (m, 1H), 2.15-2.30 (m, 2H), 2.05 (s, 3H), 1.70-1.82 (m, 4H).

### Example 138: 3-[2-({4-[(2S)-2-(4-chloro-2-fluorophenyl)-2-methyl-2H-1,3-benzodioxol-4-yl]piperidin-1-yl}methyl)-3-[(2R)-2-methoxypropyl]-3H-imidazo[4,5-b]pyridin-6-yl]-4,5-dihydro-1,2,4-oxadiazol-5-one (Compound 397a)

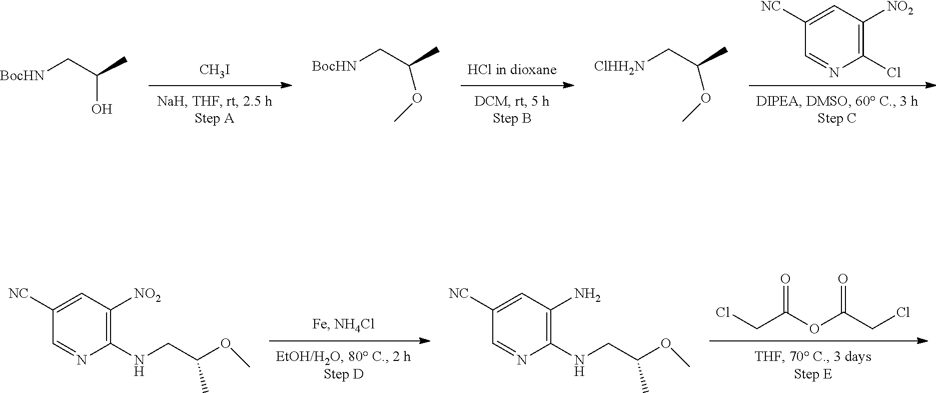

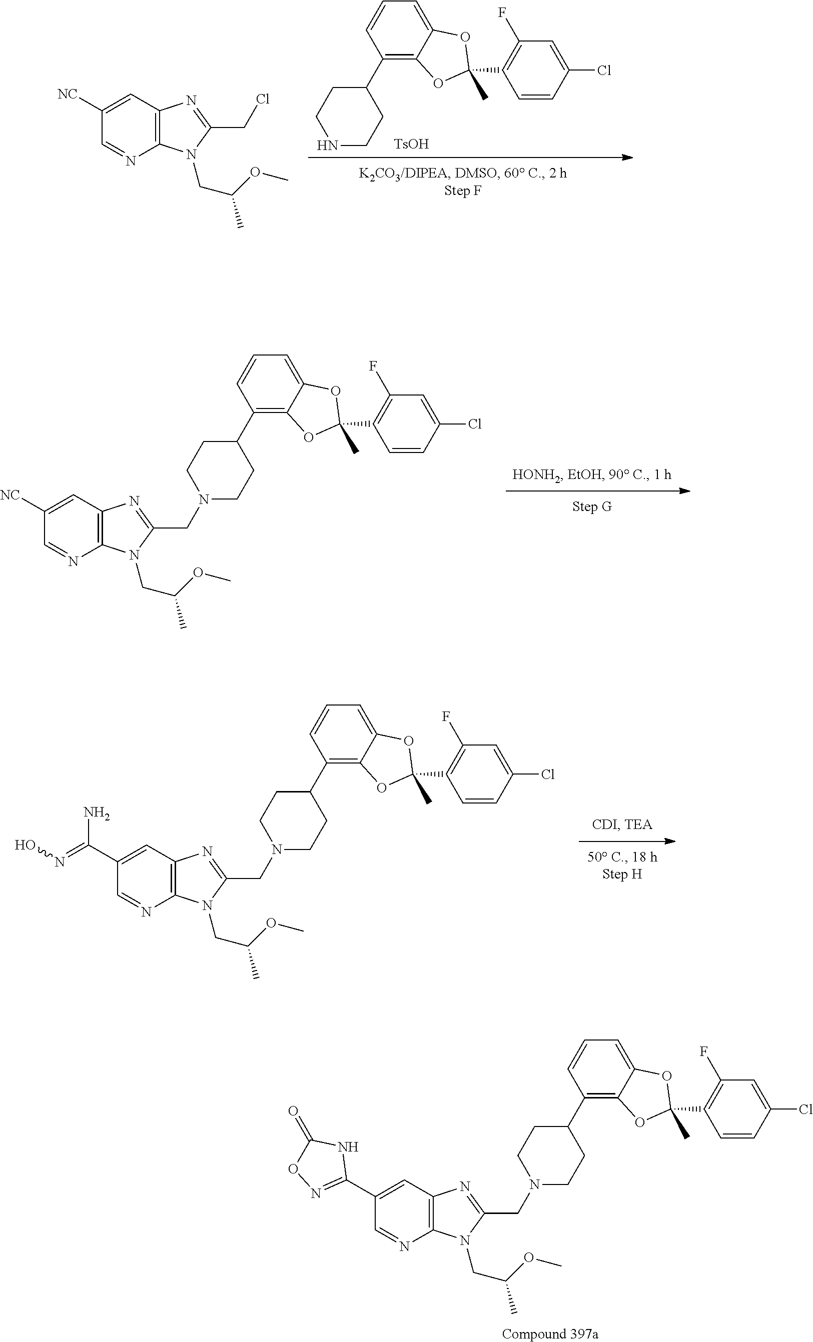

### Step A: tert-butyl (R)-(2-methoxypropyl)carbamate

To a stirred solution of tert-butyl (R)-(2-hydroxypropyl)carbamate (4.0 g, 22.8 mmol) in THF (50 mL) was added NaH (1.1 g, 60% w/w, 27.5 mmol) in portions at room temperature. The reaction mixture was stirred at room temperature for 0.5 hour. To the above mixture was added CH₃I (3.6 g, 25.1 mmol) dropwise at room temperature. The final reaction mixture was stirred at room temperature for 2.5 hours. The reaction mixture was quenched by the addition of saturated NH₄Cl (100 mL) and extracted with EA (35 mL×3). The combined organic layers were dried over anhydrous Na₂SO₄ and concentrated under vacuum. The residue was purified by a silica gel column to furnish tert-butyl (R)-(2-methoxypropyl)carbamate (3.2 g, yield: 74%) as a yellow oil.

¹H NMR (400 MHz, CDCl₃): δ 4.88 (brs, 1H), 3.43-3.28 (m, 5H), 3.04-2.98 (m, 1H), 1.46 (s, 9H), 1.10 (d, J=5.2 Hz, 3H).

### Step B: (R)-2-methoxypropan-1-amine hydrochloride

A mixture of HCl in dioxane (10 mL, 4 M) and tert-butyl (R)-(2-methoxypropyl)carbamate (3.1 g, 16.5 mmol) in DCM (10 ml) was stirred at room temperature for 5 hours. The reaction mixture was concentrated under vacuum to furnish (R)-2-methoxypropan-1-amine hydrochloride (1.9 g, yield: 94%) as a white solid.

¹H NMR (400 MHz, DMSO-d₆): δ 8.14 (s, 3H), 3.61-3.54 (m, 1H), 3.35 (s, 3H), 2.88 (dd, J=13.6, 3.2 Hz, 1H), 2.69 (dd, J=13.6, 8.4 Hz, 1H), 1.09 (d, J=6.4 Hz, 3H).

### Step C: (R)-6-((2-methoxypropyl)amino)-5-nitronicotinonitrile

To a stirred mixture of (R)-2-methoxypropan-1-amine hydrochloride (369 mg) and DIPEA (3.0 mL) in DMSO (6.0 ml) was added 6-chloro-5-nitronicotinonitrile (450 mg, 2.4 mmol) at room temperature. The reaction mixture was stirred at 60° C. for 3 hours. Upon cooling down, the mixture was diluted with H₂O (100 mL) and extracted with EA (35 mL×3). The combined organic layers were washed with brine (25 mL), dried over anhydrous Na₂SO₄, and concentrated under vacuum to furnish (R)-6-((2-methoxypropyl)amino)-5-nitronicotinonitrile (415 mg, crude) as crude yellow solid. MS Calcd.: 236.1; MS Found: 237.2 [M+H]⁺.

### Step D: (R)-5-amino-6-((2-methoxypropyl)amino)nicotinonitrile

A mixture of (R)-6-((2-methoxypropyl)amino)-5-nitronicotinonitrile (410 mg), Fe (487 mg, 8.7 mmol) and saturated aqueous NH₄Cl (5.0 mL) in EtOH (10 mL) was stirred at 80° C. for 2 hours. Upon cooling down, the mixture was diluted with H₂O (100 mL) and extracted with DCM/MeOH=10/1 (25 mL×3). The combined organic layers were dried over anhydrous Na₂SO₄ and concentrated under vacuum. The residue was purified by silica gel chromatography to furnish (R)-5-amino-6-((2-methoxypropyl)amino)nicotinonitrile (250 mg, yield: 70%) as a yellow solid. MS Calcd.: 206.1; MS Found: 207.0 [M+H]⁺.

### Step E: (R)-2-(chloromethyl)-3-(2-methoxypropyl)-3H-imidazo[4,5-b]pyridine-6-carbonitrile

To a stirred solution of (R)-5-amino-6-((2-methoxypropyl)amino)nicotinonitrile (250 mg, 1.2 mmol) in THE (6.0 mL) was added a solution of 2-chloroacetic anhydride (276 mg, 1.3 mmol) in anhydrous THE (2 mL) dropwisely at room temperature. The reaction mixture was stirred at 70° C. for 18 hours. After cooled down to room temperature, an additional portion of 2-chloroacetic anhydride (276 mg, 1.3 mmol) in anhydrous THE (2 mL) was added dropwisely. The reaction mixture was stirred at 70° C. for an additional 54 hours. Upon cooling down, the mixture was diluted with H₂O (100 mL) and extracted with EA (35 mL×3). The combined organic layers were dried over anhydrous Na₂SO₄ and concentrated under vacuum. The residue was purified by Prep-TLC to furnish (R)-2-(chloromethyl)-3-(2-methoxypropyl)-3H-imidazo[4,5-b]pyridine-6-carbonitrile (263 mg, 82%) as a yellow oil. MS Calcd.: 264.1; MS Found: 265.1 [M+H]⁺.

### Step F: 2-((4-((S)-2-(4-chloro-2-fluorophenyl)-2-methylbenzo[d][1,3]dioxol-4-yl)piperidin-1-yl)methyl)-3-((R)-2-methoxypropyl)-3H-imidazo[4,5-b]pyridine-6-carbonitrile

To a stirred mixture of (S)-4-(2-(4-chloro-2-fluorophenyl)-2-methylbenzo[d][1,3]dioxol-4-yl)piperidine-4-methylbenzenesulfonate (315 mg, 0.6 mmol), DIPEA (2.0 mL) and K₂CO₃ (167 mg, 1.2 mmol) in DMSO (5.0 mL) was added (R)-2-(chloromethyl)-3-(2-methoxypropyl)-3H-imidazo[4,5-b]pyridine-6-carbonitrile (160 mg, 0.6 mmol) at room temperature. The reaction mixture was stirred at 60° C. for 2 hours. Upon cooling down, the mixture was diluted with H₂O (100 mL) and extracted with EA (35 mL×3). The combined organic layers were washed with brine (25 mL), dried over anhydrous Na₂SO₄, and concentrated under vacuum. The residue was purified by Prep-TLC to furnish 2-((4-((S)-2-(4-chloro-2-fluorophenyl)-2-methylbenzo[d][1,3]dioxol-4-yl)piperidin-1-yl)methyl)-3-((R)-2-methoxypropyl)-3H-imidazo[4,5-b]pyridine-6-carbonitrile (213 mg, 61%) as a white solid. MS Calcd.: 575.2; MS Found: 576.2 [M+H]⁺.

### Step G: 2-((4-((S)-2-(4-chloro-2-fluorophenyl)-2-methylbenzo[d][1,3]dioxol-4-yl)piperidin-1-yl)methyl)-N-hydroxy-3-((R)-2-methoxypropyl)-3H-imidazo[4,5-b]pyridine-6-carboximidamide

A mixture of hydroxylamine (120 mg, 3.6 mmol, 50% w/w in H₂O) and 2-((4-((S)-2-(4-chloro-2-fluorophenyl)-2-methylbenzo[d][1,3]dioxol-4-yl)piperidin-1-yl)methyl)-3-((R)-2-methoxypropyl)-3H-imidazo[4,5-b]pyridine-6-carbonitrile (210 mg, 0.36 mmol) in EtOH (4.0 mL) was stirred at 90° C. for 1 hour in a sealed tube. Upon cooling down, the reaction mixture was concentrated under vacuum to furnish 2-((4-((S)-2-(4-chloro-2-fluorophenyl)-2-methylbenzo[d][1,3]dioxol-4-yl)piperidin-1-yl)methyl)-N-hydroxy-3-((R)-2-methoxypropyl)-3H-imidazo[4,5-b]pyridine-6-carboximidamide (232 mg, crude) as crude white solid. MS Calcd.: 608.2; MS Found: 609.3 [M+H]⁺.

### Step H: 3-[2-({4-[(2S)-2-(4-chloro-2-fluorophenyl)-2-methyl-2H-1,3-benzodioxol-4-yl]piperidin-1-yl}methyl)-3-[(2R)-2-methoxypropyl]-3H-imidazo[4,5-b]pyridin-6-yl]-4,5-dihydro-1,2,4-oxadiazol-5-one (Compound 397a)

A mixture of CDI (80 mg, 0.5 mmol) and 2-((4-((S)-2-(4-chloro-2-fluorophenyl)-2-methylbenzo[d][1,3]dioxol-4-yl)piperidin-1-yl)methyl)-N-hydroxy-3-((R)-2-methoxypropyl)-3H-imidazo[4,5-b]pyridine-6-carboximidamide (75 mg) in THE (5.0 mL) was stirred at 50° C. for 18 hours. Upon cooling down, the reaction mixture was concentrated under vacuum. The residue was purified by Prep-HPLC (0.1% NH₄HCO₃) to furnish 3-[2-({4-[(2S)-2-(4-chloro-2-fluorophenyl)-2-methyl-2H-1,3-benzodioxol-4-yl]piperidin-1-yl}methyl)-3-[(2R)-2-methoxypropyl]-3H-imidazo[4,5-b]pyridin-6-yl]-4,5-dihydro-1,2,4-oxadiazol-5-one (Compound 397a) (43 mg, 56%) as a white solid.

MS Calcd.: 634.2; MS Found: 635.2 [M+H]⁺.

¹H NMR (400 MHz, MeOD): δ 8.84 (d, J=2.0 Hz, 1H), 8.41 (d, J=1.6 Hz, 1H), 7.58 (t, J=8.0 Hz, 1H), 7.27 (dd, J=10.8, 2.0 Hz, 1H), 7.20 (dd, J=8.4, 2.0 Hz, 1H), 6.80-6.67 (m, 3H), 4.67-4.51 (m, 2H), 4.29-4.22 (m, 1H), 4.05-3.88 (m, 2H), 3.27-3.10 (m, 5H), 2.82-2.70 (m, 1H), 2.59-2.40 (m, 2H), 2.10-1.80 (m, 7H), 1.26 (d, J=6.0 Hz, 3H). ¹⁹F-NMR (377 MHz): −112.29.

### Example 139: 4-[(2S)-2-(4-chloro-2-fluorophenyl)-2-methyl-2H-1,3-benzodioxol-4-yl]-1-({3-[(2R)-2-methoxypropyl]-6-[5-(trifluoromethyl)-4H-1,2,4-triazol-3-yl]-3H-imidazo[4,5-b]pyridin-2-yl}methyl)piperidine (Compound 247b)

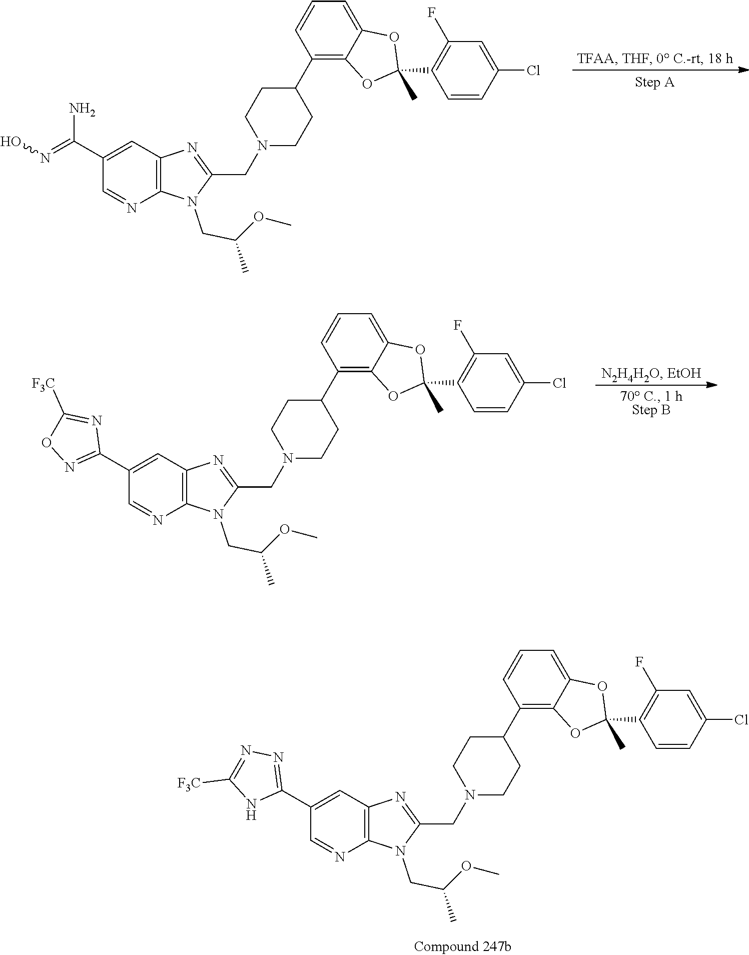

### Step A: 3-(2-((4-((S)-2-(4-chloro-2-fluorophenyl)-2-methylbenzo[d][1,3]dioxol-4-yl)piperidin-1-yl)methyl)-3-((R)-2-methoxypropyl)-3H-imidazo[4,5-b]pyridin-6-yl)-5-(trifluoromethyl)-1,2,4-oxadiazole

To a stirred solution of 2-((4-((S)-2-(4-chloro-2-fluorophenyl)-2-methylbenzo[d][1,3]dioxol-4-yl)piperidin-1-yl)methyl)-3-((R)-2-methoxypropyl)-3H-imidazo[4,5-b]pyridine-6-carbonitrile (120 mg, 0.2 mmol) in THF (6.0 mL) was added dropwisely a solution of TFAA (207 mg, 1.0 mmol) in THF (2.0 mL) at 0° C. The reaction mixture was stirred at room temperature for 18 hours. The reaction mixture was quenched by addition of saturated NaHCO₃ (75 mL) and extracted with EA (30 mL×3). The combined organic layers were dried over anhydrous Na₂SO₄ and concentrated under vacuum to furnish 3-(2-((4-((S)-2-(4-chloro-2-fluorophenyl)-2-methylbenzo[d][1,3]dioxol-4-yl)piperidin-1-yl)methyl)-3-((R)-2-methoxypropyl)-3H-imidazo[4,5-b]pyridin-6-yl)-5-(trifluoromethyl)-1,2,4-oxadiazole (150 mg, crude) as crude white solid. MS Calcd.: 686.2; MS Found: 687.3 [M+H]⁺.

### Step B: 4-[(2S)-2-(4-chloro-2-fluorophenyl)-2-methyl-2H-1,3-benzodioxol-4-yl]-1-({3-[(2R)-2-methoxypropyl]-6-[5-(trifluoromethyl)-4H-1,2,4-triazol-3-yl]-3H-imidazo[4,5-b]pyridin-2-yl}methyl)piperidine (Compound 247b)

A mixture of N₂H₄H₂O (0.2 mL) and 3-(2-((4-((S)-2-(4-chloro-2-fluorophenyl)-2-methylbenzo[d][1,3]dioxol-4-yl)piperidin-1-yl)methyl)-3-((R)-2-methoxypropyl)-3H-imidazo[4,5-b]pyridin-6-yl)-5-(trifluoromethyl)-1,2,4-oxadiazole (150 mg, crude) in EtOH (1.5 mL) was stirred at 70° C. for 1 hour. Upon cooling down, the reaction mixture was directly purified by Prep-HPLC to furnish 4-[(2S)-2-(4-chloro-2-fluorophenyl)-2-methyl-2H-1,3-benzodioxol-4-yl]-1-({3-[(2R)-2-methoxypropyl]-6-[5-(trifluoromethyl)-4H-1,2,4-triazol-3-yl]-3H-imidazo[4,5-b]pyridin-2-yl}methyl)piperidine (Compound 247b) (66 mg, 44%) as a white solid.

MS Calcd.: 685.2; MS Found: 686.2 [M+H]⁺.

¹H NMR (400 MHz, MeOD): δ 9.09 (d, J=2.0 Hz, 1H), 8.72 (d, J=1.6 Hz, 1H), 7.63 (t, J=8.4 Hz, 1H), 7.30 (dd, J=10.8, 2.0 Hz, 1H), 7.23 (dd, J=7.6, 1.2 Hz, 1H), 6.90-6.78 (m, 3H), 4.97-4.88 (m, 2H), 4.64 (dd, J=15.2, 2.4 Hz, 1H), 4.34 (dd, J=14.8, 8.8 Hz, 1H), 4.10-3.90 (m, 2H), 3.84-3.75 (m, 1H), 3.53-3.40 (m, 2H), 3.24 (s, 3H), 3.19-3.08 (m, 1H), 2.40-2.25 (m, 2H), 2.23-2.10 (m, 2H), 2.06 (s, 3H), 1.32 (d, J=6.0 Hz, 3H). ¹⁹F-NMR (377 MHz): −66.75, −112.21.

### Example 140: 3-[2-({4-[(2S)-2-(4-chloro-2-fluorophenyl)-2-methyl-2H-1,3-benzodioxol-4-yl]piperidin-1-yl}methyl)-3-{[(3S)-oxolan-3-yl]methyl}-3H-imidazo[4,5-b]pyridin-6-yl]-4,5-dihydro-1,2,4-oxadiazol-5-one (Compound 398a)

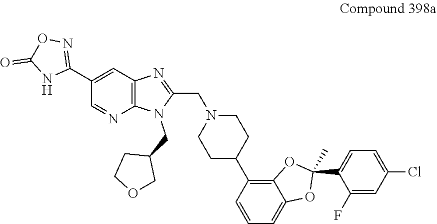 [SMILES: C[C@]1(c2ccc(Cl)cc2F)Oc2cccc(C3CCN(Cc4nc5cc(-c6noc(=O)[nH]6)cnc5n4C[C@@H]4CCOC4)CC3)c2O1]

Starting from (S)-5-nitro-6-(((tetrahydrofuran-3-yl)methyl)amino)nicotinonitrile, 3-[2-({4-[(2S)-2-(4-chloro-2-fluorophenyl)-2-methyl-2H-1,3-benzodioxol-4-yl]piperidin-1-yl}methyl)-3-{[(3S)-oxolan-3-yl]methyl}-3H-imidazo[4,5-b]pyridin-6-yl]-4,5-dihydro-1,2,4-oxadiazol-5-one (Compound 398a) (18 mg, 32%) was obtained with the similar procedure of Compound 397a as a white solid.

MS Calcd.: 646.2; MS Found: 647.3 [M+H]⁺.

¹H NMR (400 MHz, MeOD): δ 8.84 (d, J=2.0 Hz, 1H), 8.39 (d, J=2.0 Hz, 1H), 7.57 (t, J=8.0 Hz, 1H), 7.27 (dd, J=10.8, 2.0 Hz, 1H), 7.20 (dd, J=8.4, 2.4 Hz, 1H), 6.77 (t, J=8.0 Hz, 1H), 6.73-6.67 (m, 2H), 4.54 (d, J=7.6 Hz, 2H), 4.05-3.95 (m, 3H), 3.80-3.73 (m, 3H), 3.35-3.19 (m, 1H), 3.14-3.05 (m, 2H), 2.79-2.68 (m, 1H), 2.44-2.35 (m, 2H), 2.09-1.77 (m, 9H). ¹⁹F-NMR (377 MHz): −112.30.

### Example 1411-{[2-({4-[(2S)-2-(4-chloro-2-fluorophenyl)-2-methyl-2H-1,3-benzodioxol-4-yl]piperidin-1-yl}methyl)-6-(5-oxo-4,5-dihydro-1,2,4-oxadiazol-3-yl)-3H-imidazo[4,5-b]pyridin-3-yl]methyl}cyclopropane-1-carbonitrile (Compound 334a)

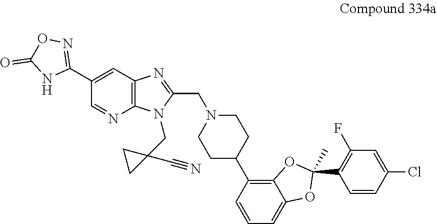 [SMILES: C[C@]1(c2ccc(Cl)cc2F)Oc2cccc(C3CCN(Cc4nc5cc(-c6noc(=O)[nH]6)cnc5n4CC4(C#N)CC4)CC3)c2O1]

Starting from 6-(((1-cyanocyclopropyl)methyl)amino)-5-nitronicotinonitrile, 1-{[2-({4-[(2S)-2-(4-chloro-2-fluorophenyl)-2-methyl-2H-1,3-benzodioxol-4-yl]piperidin-1-yl}methyl)-6-(5-oxo-4,5-dihydro-1,2,4-oxadiazol-3-yl)-3H-imidazo[4,5-b]pyridin-3-yl]methyl}cyclopropane-1-carbonitrile (Compound 334a) (12 mg) was obtained with the similar procedure of Compound 397a as a white solid.

MS Calcd.: 641.2; MS Found: 642.5 [M+H]⁺.

¹H NMR (400 MHz, DMSO-d₆): δ 8.80 (d, J=1.6 Hz, 1H), 8.42 (d, J=2.0 Hz, 1H), 7.52-7.60 (m, 2H), 7.33 (dd, J=8.4 Hz, J=1.6 Hz, 1H), 6.72-6.82 (m, 3H), 4.74 (s, 2H), 4.03 (s, 2H), 2.96-3.04 (m, 2H), 2.63-2.77 (m, 1H), 2.22-2.35 (m, 2H), 2.02 (s, 3H), 1.70-1.87 (m, 4H), 1.47-1.53 (m, 2H), 1.37-1.43 (m, 2H). ¹⁹F-NMR (377 MHz): −110.77.

### Example 142: 3-[2-({4-[2-(4-chloro-2-fluorophenyl)-2-methyl-2H-1,3-benzodioxol-4-yl]piperidin-1-yl}methyl)-3-{[(2R)-oxolan-2-yl]methyl}-3H-imidazo[4,5-b]pyridin-6-yl]-4,5-dihydro-1,2,4-oxadiazol-5-one (Compound 399a)

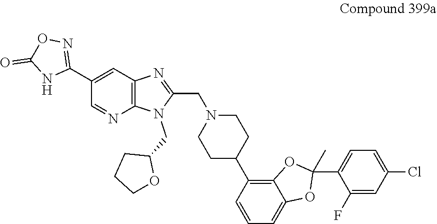 [SMILES: CC1(c2ccc(Cl)cc2F)Oc2cccc(C3CCN(Cc4nc5cc(-c6noc(=O)[nH]6)cnc5n4C[C@H]4CCCO4)CC3)c2O1]

Starting form (R)-5-nitro-6-(((tetrahydrofuran-2-yl)methyl)amino)nicotinonitrile, 3-[2-({4-[2-(4-chloro-2-fluorophenyl)-2-methyl-2H-1,3-benzodioxol-4-yl]piperidin-1-yl}methyl)-3-{[(2R)-oxolan-2-yl]methyl}-3H-imidazo[4,5-b]pyridin-6-yl]-4,5-dihydro-1,2,4-oxadiazol-5-one (Compound 399a) (35 mg) was obtained with the similar procedure of Compound 397a as white solid.

MS Calcd.: 646.2; MS Found: 647.3 [M+H]⁺.

¹H NMR (400 MHz, MeOD): δ 8.84 (d, J=1.6 Hz, 1H), 8.40 (d, J=2.0 Hz, 1H), 7.58 (t, J=8.4 Hz, 1H), 7.27 (dd, J=1.6, 10.8 Hz, 1H), 7.21 (dd, J=2.0, 8.4 Hz, 1H), 6.80-6.76 (m, 1H), 6.75-6.69 (m, 2H), 4.67 (d, J=6.0 Hz, 2H), 4.50-4.40 (m, 1H), 4.24 (dd, J=1.6, 14.0 Hz, 1H), 4.09 (d, J=14.4 Hz, 1H), 3.94-3.88 (m, 1H), 3.78-3.70 (m, 1H), 3.28-3.20 (m, 1H), 3.20-3.10 (m, 1H), 2.82-2.74 (m, 1H), 2.60-2.42 (m, 2H), 2.20-2.09 (m, 1H), 2.03 (s, 3H), 2.02-1.70 (m, 7H). ¹⁹F-NMR (377 MHz): −112.31.

### Example 143: 3-[2-({4-[2-(4-chloro-2-fluorophenyl)-2-methyl-2H-1,3-benzodioxol-4-yl]piperidin-1-yl}methyl)-3-{[(3R)-oxolan-3-yl]methyl}-3H-imidazo[4,5-b]pyridin-6-yl]-4,5-dihydro-1,2,4-oxadiazol-5-one (Compound 400a)

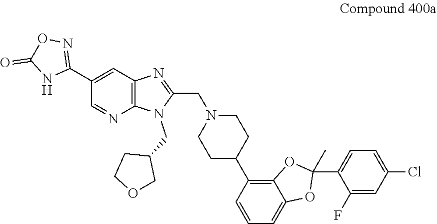 [SMILES: CC1(c2ccc(Cl)cc2F)Oc2cccc(C3CCN(Cc4nc5cc(-c6noc(=O)[nH]6)cnc5n4C[C@H]4CCOC4)CC3)c2O1]

Starting from (R)-(tetrahydrofuran-3-yl)methanamine

3-[2-({4-[2-(4-chloro-2-fluorophenyl)-2-methyl-2H-1,3-benzodioxol-4-yl]piperidin-1-yl}methyl)-3-{[(3R)-oxolan-3-yl]methyl}-3H-imidazo[4,5-b]pyridin-6-yl]-4,5-dihydro-1,2,4-oxadiazol-5-one (Compound 400a) (9.5 mg) was obtained with the similar procedure of Compound 397a as a white solid.

MS Calcd.: 646.2; MS Found: 647.3 [M+H]⁺.

¹H NMR (400 MHz, MeOD): δ 8.84 (d, J=2.0 Hz, 1H), 8.39 (d, J=1.6 Hz, 1H), 7.57 (t, J=8.0 Hz, 1H), 7.27 (dd, J=10.8 Hz, J=8.8 Hz, 1H) 7.20 (dd, J=8.4 Hz, J=6.0 Hz, 1H), 6.79-6.67 (m, 3H), 4.65-4.50 (m, 4H), 4.03-3.95 (m, 2H), 3.81-3.72 (m, 2H), 3.15-3.05 (m, 2H), 2.78-2.65 (m, 1H), 2.45-2.35 (m, 2H), 2.10-1.78 (m, 10H). ¹⁹F-NMR (377 MHz): −112.29, −112.30.

### Example 144: 4-[2-(4-chloro-2-fluorophenyl)-2-methyl-2H-1,3-benzodioxol-4-yl]-1-[(3-{[(3R)-oxolan-3-yl]methyl}-6-[5-(trifluoromethyl)-4H-1,2,4-triazol-3-yl]-3H-imidazo[4,5-b]pyridin-2-yl)methyl]piperidine (Compound 401a)

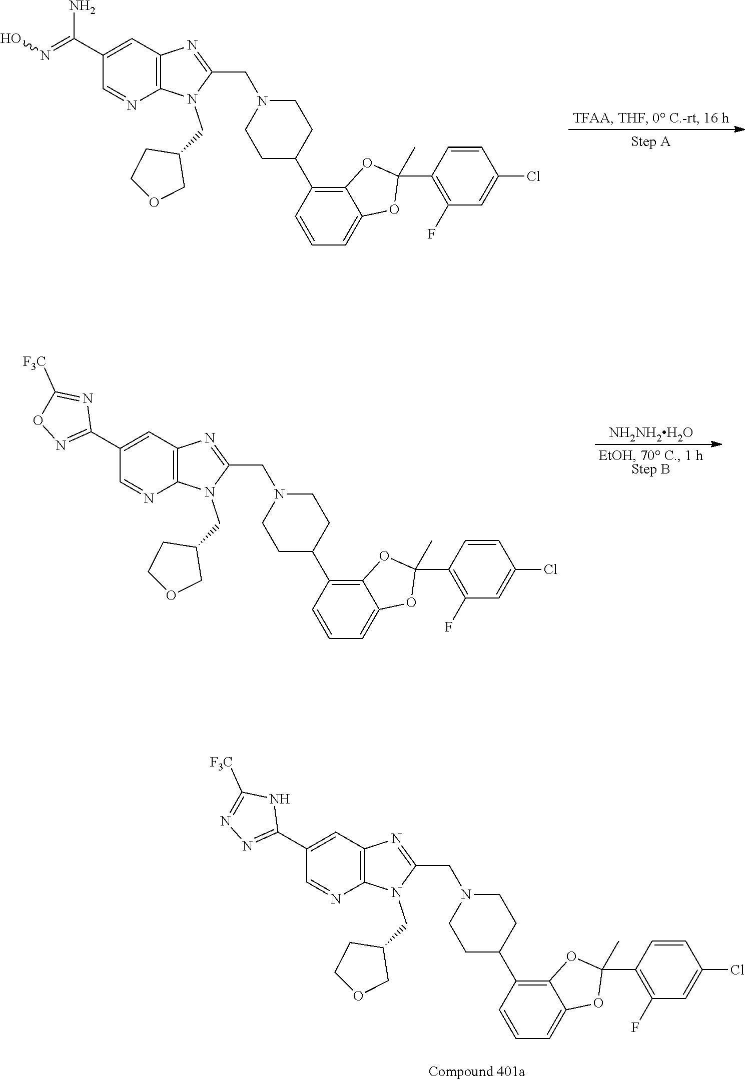

### Step A: 3-(2-((4-(2-(4-chloro-2-fluorophenyl)-2-methylbenzo[d][1,3]dioxol-4-yl)piperidin-1-yl)methyl)-3-(((R)-tetrahydrofuran-3-yl)methyl)-3H-imidazo[4,5-b]pyridin-6-yl)-5-(trifluoromethyl)-1,2,4-oxadiazole

A mixture of 2-((4-(2-(4-chloro-2-fluorophenyl)-2-methylbenzo[d][1,3]dioxol-4-yl)piperidin-1-yl)methyl)-N′-hydroxy-3-(((R)-tetrahydrofuran-3-yl)methyl)-3H-imidazo[4,5-b]pyridine-6-carboximidamide (65 mg, 0.10 mmol) in THF (6.0 mL) at 0° C. was added TFAA (110 mg, 0.52 mmol) and stirred for 10 mins. Then, the mixture was warmed to room temperature and stirred for 16 h. the mixture was diluted with saturated NaHCO₃ (100 mL) and extracted with EA (25 mL×3). The combined organic layers were dried over anhydrous Na₂SO₄ and concentrated under vacuum to yield the crude product of 3-(2-((4-(2-(4-chloro-2-fluorophenyl)-2-methylbenzo[d][1,3]dioxol-4-yl)piperidin-1-yl)methyl)-3-(((R)-tetrahydrofuran-3-yl)methyl)-3H-imidazo[4,5-b]pyridin-6-yl)-5-(trifluoromethyl)-1,2,4-oxadiazole (43 mg, yield: 59%) as a yellow oil, which was used in the next step directly without further purification. MS Calcd.: 698.2; MS Found: 699.2 [M+H]⁺.

### Step B: 4-[2-(4-chloro-2-fluorophenyl)-2-methyl-2H-1,3-benzodioxol-4-yl]-1-[(3-{[(3R)-oxolan-3-yl]methyl}-6-[5-(trifluoromethyl)-4H-1,2,4-triazol-3-yl]-3H-imidazo[4,5-b]pyridin-2-yl)methyl]piperidine (Compound 401a)

A mixture of 3-(2-((4-(2-(4-chloro-2-fluorophenyl)-2-methylbenzo[d][1,3]dioxol-4-yl)piperidin-1-yl)methyl)-3-(((R)-tetrahydrofuran-3-yl)methyl)-3H-imidazo[4,5-b]pyridin-6-yl)-5-(trifluoromethyl)-1,2,4-oxadiazole (43 mg) in EtOH (4.0 mL) at room temperature was added NH₂NH₂·H₂O (0.5 mL), the mixture was stirred at 70° C. for 1 h. Upon cooling down, the mixture was filtered and purified by Prep-HPLC (0.1% NH₄HCO₃) to furnish 4-[2-(4-chloro-2-fluorophenyl)-2-methyl-2H-1,3-benzodioxol-4-yl]-1-[(3-{[(3R)-oxolan-3-yl]methyl}-6-[5-(trifluoromethyl)-4H-1,2,4-triazol-3-yl]-3H-imidazo[4,5-b]pyridin-2-yl)methyl]piperidine (Compound 401a) (27 mg, yield: 64%) as a white solid.

MS Calcd.: 697.2; MS Found: 698.4 [M+H]⁺.

¹H NMR (400 MHz, MeOD): δ 9.05 (d, J=2.0 Hz, 1H), 8.59 (d, J=1.6 Hz, 1H), 7.57 (t, J=8.0 Hz, 1H), 7.27 (dd, J=10.8 Hz, J=8.8 Hz, 1H) 7.20 (dd, J=8.4 Hz, J=6.4 Hz, 1H), 6.79-6.68 (m, 3H), 4.55 (d, J=7.6 Hz, 2H), 4.05-3.93 (m, 3H), 3.81-3.74 (m, 3H), 3.12-3.00 (m, 2H), 2.81-2.66 (m, 1H), 2.40-2.30 (m, 2H), 2.10-1.78 (m, 10H). ¹⁹F-NMR (377 MHz): −66.66, −112.30, −112.31.

### Example 145: 4-[2-(4-chloro-2-fluorophenyl)-2-methyl-2H-1,3-benzodioxol-4-yl]-1-{[3-(2-methoxyethyl)-6-[5-(trifluoromethyl)-4H-1,2,4-triazol-3-yl]-3H-imidazo[4,5-b]pyridin-2-yl]methyl}piperidine (Compound 402)

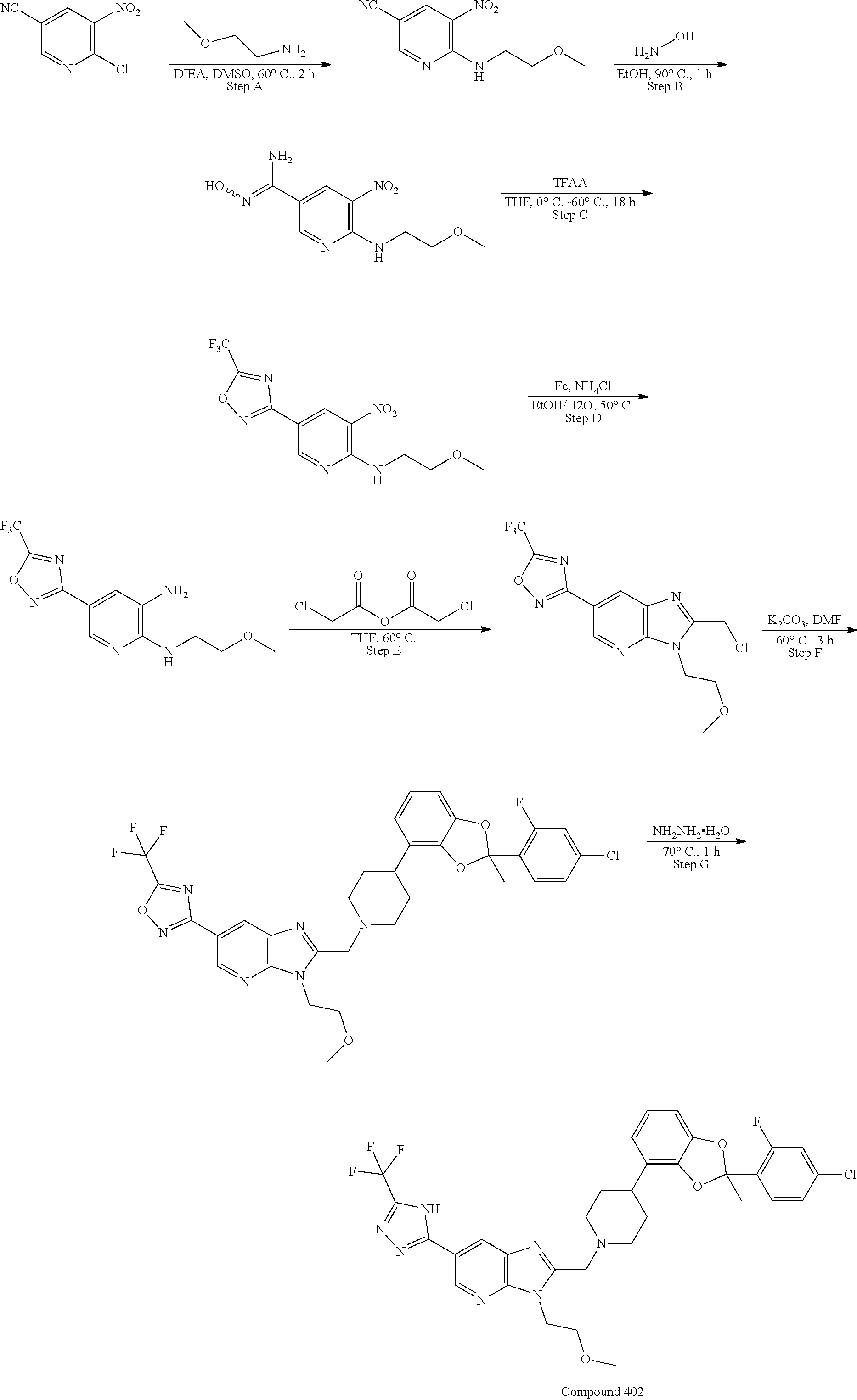

### Step A: 6-((2-methoxyethyl)amino)-5-nitronicotinonitrile

A mixture of 6-chloro-5-nitronicotinonitrile (300 mg, 1.6 mmol), 2-methoxyethanamine (148 mg, 2.0 mmol) in DMSO (5 mL) were added DIEA (2.1 g, 16.39 mmol). The reaction mixture was stirred at 60° C. for 2 hours. The mixture was poured into cold water (15 mL) and extracted with EtOAc (3×15 mL), the combined organic layers were washed with brine, dried over sodium sulfate, filtered, and concentrated under reduced pressure. The residue was purified by column chromatography to give 6-((2-methoxyethyl)amino)-5-nitronicotinonitrile (300 mg, yield: 82%) as yellow solid. MS Calcd.: 222.1; MS Found: 223.0 [M+H]⁺.

### Step B: N′-hydroxy-6-((2-methoxyethyl)amino)-5-nitronicotinimidamide

A mixture of hydroxylamine (186 mg, 13.5 mmol, 50% w/w in H₂O) and 6-((2-methoxyethyl)amino)-5-nitronicotinonitrile (300 mg, 1.35 mmol) in EtOH (3.0 mL) was stirred at 90° C. for 1 h. Upon cooling down, the mixture was concentrated under vacuum to give N′-hydroxy-6-((2-methoxyethyl)amino)-5-nitronicotinimidamide (320 mg, crude) as a crude white solid. MS Calcd.: 255.1; MS Found: 256.1 [M+H]⁺.

### Step C: N-(2-methoxyethyl)-3-nitro-5-(5-(trifluoromethyl)-1,2,4-oxadiazol-3-yl)pyridin-2-amine

To a stirred solution of N′-hydroxy-6-((2-methoxyethyl)amino)-5-nitronicotinimidamide (320 mg) in THE (10.0 mL) was added a solution of TFAA (1.3 g, 6.3 mmol) in THE (2.0 mL) dropwisely at 0° C. The final reaction mixture was stirred at room temperature for 18 hours. The reaction mixture was quenched by saturated aqueous NaHCO₃ (75 mL) and extracted with EA (30 mL×3). The combined organic layers were dried over anhydrous Na₂SO₄ and concentrated under vacuum to give N-(2-methoxyethyl)-3-nitro-5-(5-(trifluoromethyl)-1,2,4-oxadiazol-3-yl)pyridin-2-amine (210 mg, yield: 50%) as a white solid. MS Calcd.: 333.1; MS Found: 334.1 [M+H]⁺.

### Step D: N2-(2-methoxyethyl)-5-(5-(trifluoromethyl)-1,2,4-oxadiazol-3-yl)pyridine-2,3-diamine

To a solution of N-(2-methoxyethyl)-3-nitro-5-(5-(trifluoromethyl)-1,2,4-oxadiazol-3-yl)pyridin-2-amine (180 mg) in EtOH (8 mL) and H₂O (2 mL) was added Fe (151 mg, 2.7 mmol) and NH₄Cl (286 mg, 5.4 mmol). The mixture was stirred at 50° C. for 2 hours. The reaction mixture was filtered, the filtrate was poured into cold water (30 mL) and extracted with EtOAc (2×30 mL), the combined organic layers were washed with brine, dried over sodium sulfate, filtered, and concentrated under reduced pressure. The residue was purified by column chromatography to give N2-(2-methoxyethyl)-5-(5-(trifluoromethyl)-1,2,4-oxadiazol-3-yl)pyridine-2,3-diamine (110 mg, yield: 67%) as white solid. MS Calcd.: 303.1; MS Found: 304.1 [M+H]⁺.

### Step E: 3-(2-(chloromethyl)-3-(2-methoxyethyl)-3H-imidazo[4,5-b]pyridin-6-yl)-5-(trifluoromethyl)-1,2,4-oxadiazole

A mixture of N2-(2-methoxyethyl)-5-(5-(trifluoromethyl)-1,2,4-oxadiazol-3-yl)pyridine-2,3-diamine (110 mg, 0.36 mmol) in THE (5 mL) was added 2-chloroacetic anhydride (62 mg, 0.36 mmol) at 0° C. The reaction mixture was stirred at 60° C. for 18 hours. Then, another portion of 2-chloroacetic anhydride (62 mg, 0.36 mmol) was added and stirred for another 16 h. The mixture was poured into cold water (20 mL) and extracted with EtOAc (2×30 mL), the combined organic layers were washed with brine, dried over sodium sulfate, filtered, and concentrated under reduced pressure. The residue was purified by column chromatography to give 3-(2-(chloromethyl)-3-(2-methoxyethyl)-3H-imidazo[4,5-b]pyridin-6-yl)-5-(trifluoromethyl)-1,2,4-oxadiazole (60 mg, yield: 46%) as yellow solid. MS Calcd.: 361.1; MS Found: 362.1 [M+H]⁺.

### Step F: 3-(2-((4-(2-(4-chloro-2-fluorophenyl)-2-methylbenzo[d][1,3]dioxol-4-yl)piperidin-1-yl)methyl)-3-(2-methoxyethyl)-3H-imidazo[4,5-b]pyridin-6-yl)-5-(trifluoromethyl)-1,2,4-oxadiazole

To a solution of 3-(2-(chloromethyl)-3-(2-methoxyethyl)-3H-imidazo[4,5-b]pyridin-6-yl)-5-(trifluoromethyl)-1,2,4-oxadiazole (60 mg, 0.17 mmol), K₂CO₃ (69 mg, 0.50 mmol) and TEA (50 mg, 0.498 mmol) in DMF (5 mL) was stirred at room temperature for 10 mins. 2-(chloromethyl)-5-(5-methyl-4-((2-(trimethylsilyl)ethoxy)methyl)-4H-1,2,4-triazol-3-yl)-1-(oxetan-2-ylmethyl)-1H-benzo[d]imidazole (64 mg, 0.17 mmol) was added, and the reaction mixture was stirred at 60° C. for 3 hours. The mixture was poured into cold water (20 mL) and extracted with EtOAc (2×20 mL), the combined organic layers were washed with brine, dried over sodium sulfate, filtered, and concentrated under reduced pressure. The residue was purified by column chromatography to give 3-(2-((4-(2-(4-chloro-2-fluorophenyl)-2-methylbenzo[d][1,3]dioxol-4-yl)piperidin-1-yl)methyl)-3-(2-methoxyethyl)-3H-imidazo[4,5-b]pyridin-6-yl)-5-(trifluoromethyl)-1,2,4-oxadiazole (25 mg, yield: 22%) as colorless oil. MS Calcd.: 672.2; MS Found: 673.2 [M+H]⁺.

### Step G: 4-[2-(4-chloro-2-fluorophenyl)-2-methyl-2H-1,3-benzodioxol-4-yl]-1-{[3-(2-methoxyethyl)-6-[5-(trifluoromethyl)-4H-1,2,4-triazol-3-yl]-3H-imidazo[4,5-b]pyridin-2-yl]methyl}piperidine (Compound 402)

A mixture of 3-(2-((4-(2-(4-chloro-2-fluorophenyl)-2-methylbenzo[d][1,3]dioxol-4-yl)piperidin-1-yl)methyl)-3-(2-methoxyethyl)-3H-imidazo[4,5-b]pyridin-6-yl)-5-(trifluoromethyl)-1,2,4-oxadiazole (25 mg, 0.037 mmol) and NH₂NH₂·H₂O (0.2 mL) in EtOH (2 ml) was stirred at 70° C. for 1 hour. The reaction mixture was purified by prep-HPLC to give 4-[2-(4-chloro-2-fluorophenyl)-2-methyl-2H-1,3-benzodioxol-4-yl]-1-{[3-(2-methoxyethyl)-6-[5-(trifluoromethyl)-4H-1,2,4-triazol-3-yl]-3H-imidazo[4,5-b]pyridin-2-yl]methyl}piperidine (Compound 402) (14 mg, yield: 56%) as white solid.

MS Calcd.: 671.2; MS Found: 672.3 [M+H]⁺.

¹H NMR (400 MHz, MeOD): δ 9.00 (d, J=2.0 Hz, 1H), 8.57 (d, J=2.0 Hz, 1H), 7.60-7.52 (m, 2H), 7.35-7.30 (m, 1H), 6.81-6.70 (m, 3H), 4.65 (t, J=4.6 Hz, 2H), 3.92 (s, 2H), 3.82 (t, J=6.0 Hz, 2H), 3.26 (s, 3H), 3.00-2.93 (m, 2H), 2.72-2.60 (m, 1H), 2.30-2.20 (m, 2H), 2.02 (s, 3H), 1.80-1.70 (m, 4H). ¹⁹F-NMR (377 MHz): −63.58, −110.78.

### Example 146: 3-[2-({4-[(2S)-2-(4-chloro-2-fluorophenyl)-2-methyl-2H-1,3-benzodioxol-4-yl]piperidin-1-yl}methyl)-3-{[(2S)-oxetan-2-yl]methyl}-3H-imidazo[4,5-b]pyridin-6-yl]-4,5-dihydro-1,2,4-oxadiazol-5-one (Compound 262a)

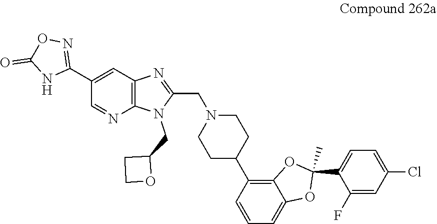 [SMILES: C[C@]1(c2ccc(Cl)cc2F)Oc2cccc(C3CCN(Cc4nc5cc(-c6noc(=O)[nH]6)cnc5n4C[C@@H]4CCO4)CC3)c2O1]

Starting from (S)-5-nitro-6-((oxetan-2-ylmethyl)amino)nicotinonitrile, 3-[2-({4-[(2S)-2-(4-chloro-2-fluorophenyl)-2-methyl-2H-1,3-benzodioxol-4-yl]piperidin-1-yl}methyl)-3-{[(2S)-oxetan-2-yl]methyl}-3H-imidazo[4,5-b]pyridin-6-yl]-4,5-dihydro-1,2,4-oxadiazol-5-one (Compound 262a) (28 mg) was obtained with the similar procedure of Compound 397a as white solid.

MS Calcd: 632.2; MS Found: 633.2 [M+H]⁺.

¹H NMR (400 MHz, DMSO-d₆) δ 8.78 (d, J=1.6 Hz, 1H), 8.38 (d, J=1.6 Hz, 1H), 7.59-7.51 (m, 2H), 7.35-7.30 (m, 1H), 6.82-6.71 (m, 3H), 5.22-5.15 (m, 1H), 4.89-4.81 (m, 1H), 4.76-4.69 (m, 1H), 4.51-4.40 (m, 1H), 4.39-4.31 (m, 1 H), 4.03-3.90 (m, 2H), 3.05-2.90 (m, 2H), 2.73-2.62 (m, 2H), 2.53-2.40 (m, 1H), 2.34-2.20 (m, 2H), 2.02 (s, 3H), 1.85-1.70 (m, 4H). ¹⁹F-NMR (377 MHz): −110.79.

### Example 147: 3-fluoro-4-(2-methyl-4-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}-2H-1,3-benzodioxol-2-yl)benzonitrile (Compound 403a)

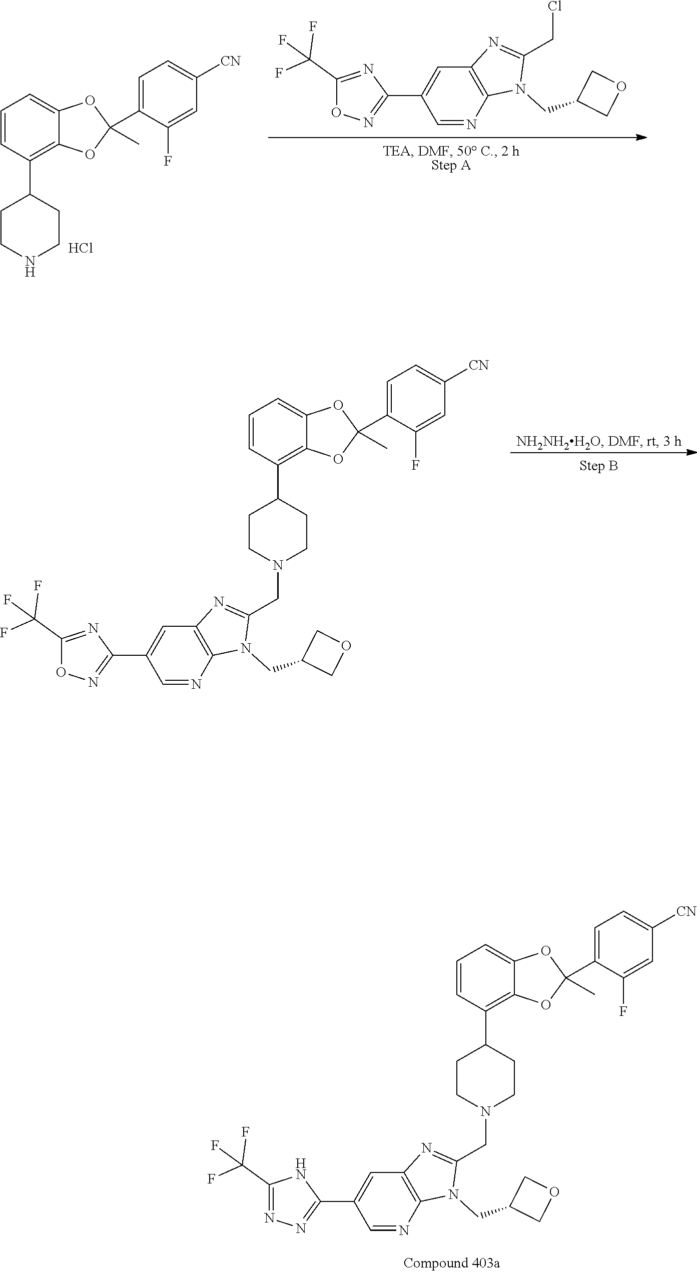

### Step A: 3-fluoro-4-(2-methyl-4-(1-((3-(((S)-oxetan-2-yl)methyl)-6-(5-(trifluoromethyl)-1,2,4-oxadiazol-3-yl)-3H-imidazo[4,5-b]pyridin-2-yl)methyl)piperidin-4-yl)benzo[d][1,3]dioxol-2-yl)benzonitrile

A mixture of (S)-3-(2-(chloromethyl)-3-(oxetan-2-ylmethyl)-3H-imidazo[4,5-b]pyridin-6-yl)-5-(trifluoro methyl)-1,2,4-oxadiazole (65 mg, 0.18 mmol), 3-fluoro-4-(2-methyl-4-(piperidin-4-yl)benzo[d][1,3] dioxol-2-yl)benzonitrile HCl salt (50 mg, 0.18 mmol) and TEA (110 mg, 1.1 mmol) in DMF (1 mL) was stirred at 50° C. for 2 hours. After the reaction was completed, the solvent was removed in vacuo. The residue was purified by column chromatography on silica gel (PE:EA=1:1) to give 3-fluoro-4-(2-methyl-4-(1-((3-(((S)-oxetan-2-yl)methyl)-6-(5-(trifluoromethyl)-1,2,4-oxadiazol-3-yl)-3H-imidazo[4,5-b]pyridin-2-yl)methyl)piperidin-4-yl)benzo[d][1,3]dioxol-2-yl)benzonitrile (60 mg, 66% yield) as yellow oil. MS Calcd.: 675.2; MS Found: 676.2 [M+H]⁺.

### Step B: 3-fluoro-4-(2-methyl-4-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}-2H-1,3-benzodioxol-2-yl)benzonitrile (Compound 403a)

To a solution of 3-fluoro-4-(2-methyl-4-(1-((3-(((S)-oxetan-2-yl)methyl)-6-(5-(trifluoromethyl)-1,2,4-oxadiazol-3-yl)-3H-imidazo[4,5-b]pyridin-2-yl)methyl)piperidin-4-yl)benzo[d][1,3]dioxol-2-yl) benzonitrile (60 mg, 0.09 mmol) in DMF (1 mL) was added NH₂NH₂·H₂O (22 mg, 0.45 mmol). The reaction was stirred at room temperature for 3 hours. After the reaction was completed, the reaction was filtered and purified by Prep-HPLC to give 3-fluoro-4-(2-methyl-4-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}-2H-1,3-benzodioxol-2-yl)benzonitrile (Compound 403a) (13 mg, 23% yield) as a white solid.

MS Calcd.: 674.2; MS Found: 675.6 [M+H]⁺.

¹H NMR (400 MHz, DMSO-d₆): δ 9.00 (d, J=2.0 Hz, 1H), 8.58 (d, J=2.0 Hz, 1H), 7.98 (d, J=10.4 Hz, 1H), 7.70-7.76 (m, 2H), 6.75-6.89 (m, 3H), 5.17-5.25 (m, 1H), 4.86 (dd, J=14.8 Hz, J=6.4 Hz, 1H), 4.73 (dd, J=14.4 Hz, J=4.0 Hz, 1H), 4.45-4.52 (m, 1H), 4.35-4.41 (m, 1H), 3.90-4.02 (m, 2H), 2.92-3.03 (m, 2H), 2.63-2.77 (m, 2H), 2.50-2.58 (m, 1H), 2.20-2.33 (m, 2H), 2.05 (s, 3H), 1.70-1.85 (m, 4H). ¹⁹F-NMR (377 MHz): −63.36, −111.08.

### Example 148: 3-[2-({4-[2-(5-chloropyridin-2-yl)-2-methyl-2H-1,3-benzodioxol-4-yl]piperidin-1-yl}methyl)-3-{[(2S)-oxetan-2-yl]methyl}-3H-imidazo[4,5-b]pyridin-6-yl]-4,5-dihydro-1,2,4-oxadiazol-5-one (Compound 332a)

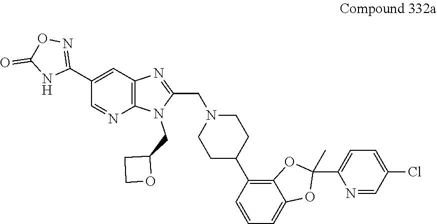 [SMILES: CC1(c2ccc(Cl)cn2)Oc2cccc(C3CCN(Cc4nc5cc(-c6noc(=O)[nH]6)cnc5n4C[C@@H]4CCO4)CC3)c2O1]

Starting from 5-chloro-2-(2-methyl-4-(piperidin-4-yl)benzo[d][1,3]dioxol-2-yl)pyridine HCl salt, 3-[2-({4-[2-(5-chloropyridin-2-yl)-2-methyl-2H-1,3-benzodioxol-4-yl]piperidin-1-yl}methyl)-3-{[(2S)-oxetan-2-yl]methyl}-3H-imidazo[4,5-b]pyridin-6-yl]-4,5-dihydro-1,2,4-oxadiazol-5-one (Compound 332a) (31 mg) was obtained with the similar procedure of Compound 397a as a white solid.

MS Calcd.: 615.2; MS Found: 616.4 [M+H]⁺.

¹H NMR (400 MHz, DMSO-d₆): δ8.89 (d, J=2.0 Hz, 1H), 8.73 (d, J=2.0 Hz, 1H), 8.55 (d, J=2.0 Hz, 1H), 8.03 (dd, J=8.8 Hz, J=2.8 Hz, 1H), 7.64 (d, J=8.4 Hz, 1H), 6.84-6.90 (m, 2H), 6.73-6.80 (m, 1H), 5.08-5.16 (m, 1H), 4.77-4.95 (m, 3H), 4.64-4.71 (m, 1H), 4.46-4.52 (m, 1H), 4.25-4.32 (m, 1H), 3.65-3.85 (m, 2H), 3.23-3.40 (m, 2H), 2.90-3.03 (m, 1H), 2.65-2.75 (m, 1H), 2.29-2.40 (m, 1H), 1.95-2.18 (m, 7H).

### Example 149A: 5-chloro-2-(2-methyl-4-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}-2H-1,3-benzodioxol-2-yl)pyridine (Compound 333a); Isomer 1 (Compound 333b) and Isomer 2 (Compound 333c)

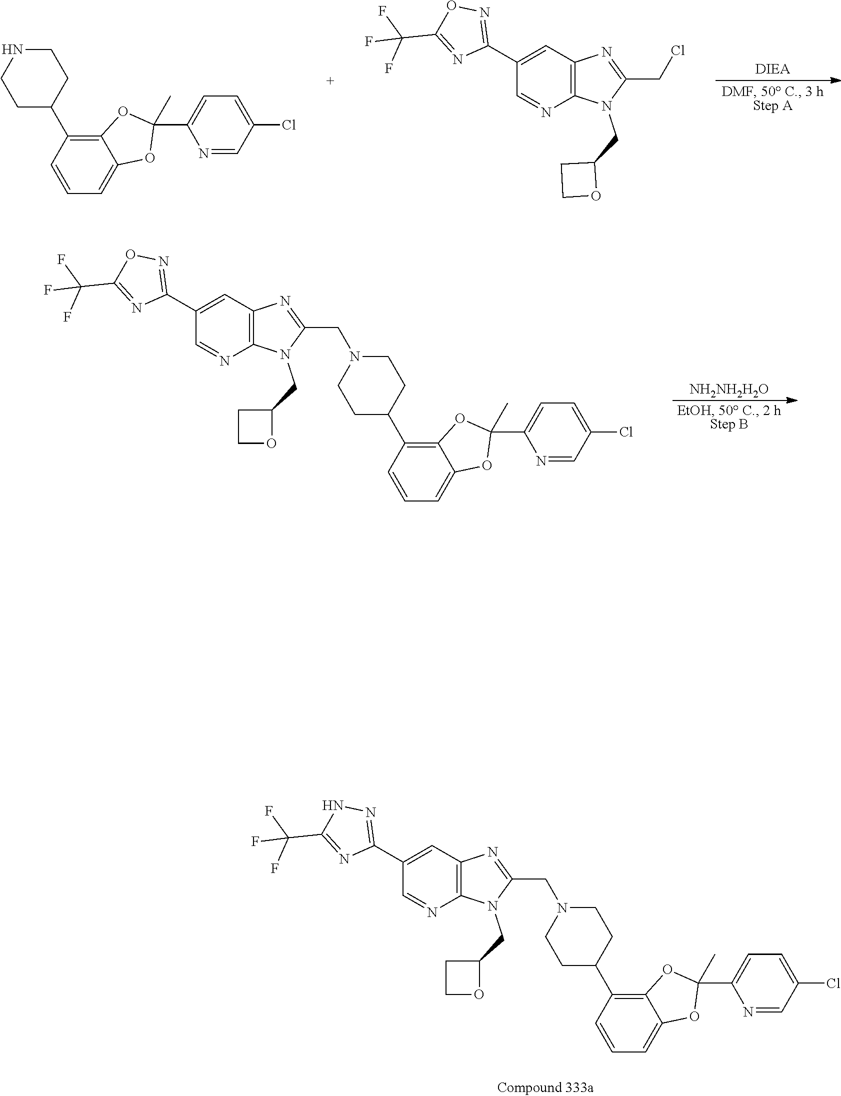

### Step A: 3-(2-((4-(2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-b]pyridin-6-yl)-5-(trifluoromethyl)-1,2,4-oxadiazole

A mixture of 5-chloro-2-(2-methyl-4-(piperidin-4-yl)benzo[d][1,3]dioxol-2-yl)pyridine (44 mg, 0.13 mmol), DIEA (52 mg, 0.39 mmol) in DMF (1 mL) was stirred at room temperature for 10 minutes. The mixture was added with (S)-3-(2-(chloromethyl)-3-(oxetan-2-ylmethyl)-3H-imidazo[4,5-b]pyridin-6-yl)-5-(trifluoromethyl)-1,2,4-oxadiazole (50 mg, 0.13 mmol) and stirred at 50° C. for 3 hours. After the reaction was completed, the mixture was diluted with EtOAc (20 mL), washed with H₂O (10 mL×3). The organic layer was dried over anhydrous sodium sulfate, filtered, and concentrated in vacuum. The residue was purified by column chromatography (PE/EA=1/1) to give 3-(2-((4-(2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-b]pyridin-6-yl)-5-(trifluoromethyl)-1,2,4-oxadiazole (70 mg, yield: 77%) as a yellow solid. MS Calcd.: 667.2; MS Found: 668.3[M+H]⁺.

### Step B: 5-chloro-2-(2-methyl-4-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}-2H-1,3-benzodioxol-2-yl)pyridine (Compound 333a)

A mixture of 3-(2-((4-(2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-b]pyridin-6-yl)-5-(trifluoromethyl)-1,2,4-oxadiazole (70 mg, 0.10 mmol) and NH₂NH₂·H₂O (15 mg, 0.30 mmol) in EtOH (1 mL) was stirred at 50° C. for 2 hours. After the reaction was completed. The reaction mixture was purified directly by prep-HPLC to give 5-chloro-2-(2-methyl-4-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}-2H-1,3-benzodioxol-2-yl)pyridine (Compound 333a) (15 mg, yield: 22%) as white solid.

MS Calcd.: 666.2; MS Found: 667.4 [M+H]⁺.

¹H NMR (400 MHz, DMSO-d₆): δ 8.99 (d, J=1.6 Hz, 1H), 8.72 (d, J=2.4 Hz, 1H), 8.59 (d, J=2.0 Hz, 1H), 7.99-8.02 (m, 1H), 7.60 (d, J=8.4 Hz, 1H), 6.73-6.85 (m, 3H), 5.16-5.23 (m, 1H), 4.80-4.88 (m, 1H), 4.69-4.74 (m, 1H), 4.43-4.50 (m, 1H), 4.34-4.40 (m, 1H), 3.90-4.02 (m, 2H), 2.93-3.04 (m, 2H), 2.62-2.73 (m, 2H), 2.44-2.55 (m, 1H), 2.20-2.33 (m, 2H), 2.01 (s, 3H), 1.70-1.83 (m, 4H). ¹⁹F-NMR (377 MHz): −63.65.

### Example 149B and Example 149C 5-chloro-2-[(2S)-2-methyl-4-{1-[(3-{[(2S)-oxetan-2-yl]methyl}-6-[5-(trifluoromethyl)-1H-1,2,4-triazol-3-yl]-3H-imidazo[4,5-b]pyridin-2-yl)methyl]piperidin-4-yl}-2H-1,3-benzodioxol-2-yl]pyridine (isomer 1: Compound 333b; isomer 2: Compound 333c)

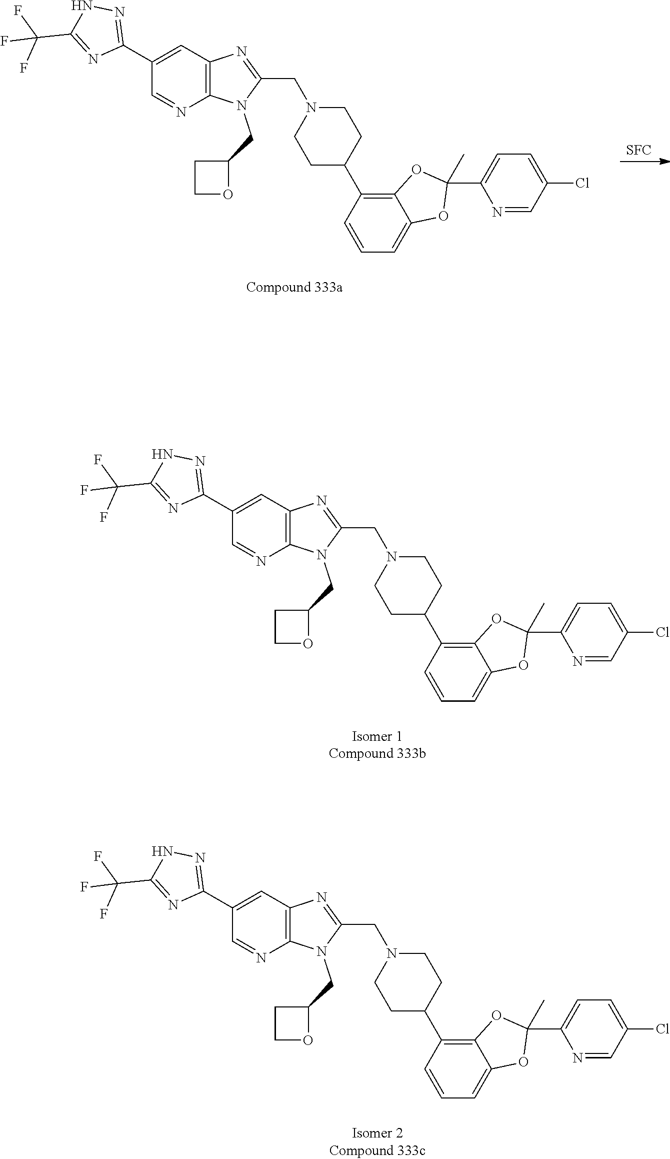 [SMILES: CC1(c2ccc(Cl)cn2)Oc2cccc(C3CCN(Cc4nc5cc(-c6n[nH]c(C(F)(F)F)n6)cnc5n4C[C@@H]4CCO4)CC3)c2O1 ; "SFC" ; CC1(c2ccc(Cl)cn2)Oc2cccc(C3CCN(Cc4nc5cc(-c6n[nH]c(C(F)(F)F)n6)cnc5n4C[C@@H]4CCO4)CC3)c2O1 ; CC1(c2ccc(Cl)cn2)Oc2cccc(C3CCN(Cc4nc5cc(-c6n[nH]c(C(F)(F)F)n6)cnc5n4C[C@@H]4CCO4)CC3)c2O1]

The product of Compound 333a (160 mg, 0.24 mmol) was purified directly by SFC (Column: DAICELCHIRALCEL® OJ, 250*25 mm 10 μm; Mobile Phase A: Supercritical CO₂, Mobile Phase B: MEOH (+0.1% 7.0 mol/l Ammonia in MEOH), A:B=80:20; Flow: 80 mL/min, Back Pressure: 100 Bar; cycle time: 1.2 min) to give Compound 333b (19 mg, yield: 12%) and Compound 333c (30 mg, yield: 19%) as white solid.

SFC analytical method:

Column: DAICEL CHIRALPAK® OJ, 100*3.0 mm 3 μm; Mobile Phase A: Supercritical CO₂, Mobile Phase B: MeOH (0.1% DEA); Flow: 1.5 mL/min, Back Pressure: 1800 psi; Run time: 4 min

Compound 333b:

T_{R}=2.987 min.

MS Calcd.: 666.2; MS Found: 667.5 [M+H]⁺.

¹H NMR (400 MHz, DMSO-d₆): δ 8.99 (d, J=2.0 Hz, 1H), 8.72 (d, J=2.0 Hz, 1H), 8.59 (d, J=2.0 Hz, 1H), 8.00 (dd, J=8.4, 2.4 Hz, 1H), 7.61 (d, J=8.4 Hz, 1H), 6.74-6.83 (m, 3H), 5.15-5.25 (m, 1H), 4.80-4.88 (m, 1H), 4.68-4.75 (m, 1H), 4.43-4.50 (m, 1H), 4.33-4.40 (m, 1H), 3.90-4.02 (m, 2H), 2.90-3.05 (m, 2H), 2.60-2.73 (m, 2H), 2.42-2.54 (m, 1H), 2.20-2.32 (m, 2H), 2.01 (s, 3H), 1.70-1.82 (m, 4H). ¹⁹F-NMR (377 MHz): −63.63.

Compound 333c:

T_{R}=2.997 min.

MS Calcd.: 666.2; MS Found: 667.1 [M+H]⁺.

¹H NMR (400 MHz, DMSO-d₆): δ 8.99 (d, J=1.6 Hz, 1H), 8.72 (d, J=2.4 Hz, 1H), 8.60 (d, J=1.6 Hz, 1H), 8.00 (dd, J=8.4, 2.4 Hz, 1H), 7.60 (d, J=8.4 Hz, 1H), 6.74-6.83 (m, 3H), 5.16-5.24 (m, 1H), 4.80-4.90 (m, 1H), 4.69-4.76 (m, 1H), 4.42-4.50 (m, 1H), 4.35-4.40 (m, 1H), 3.92-4.05 (m, 2H), 2.92-3.04 (m, 2H), 2.60-2.74 (m, 2H), 2.42-2.51 (m, 1H), 2.20-2.33 (m, 2H), 2.01 (s, 3H), 1.72-1.82 (m, 4H). ¹⁹F-NMR (377 MHz): −63.70.

### Example 150: 2-({4-[2-(2-chloro-4-fluorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 404a)

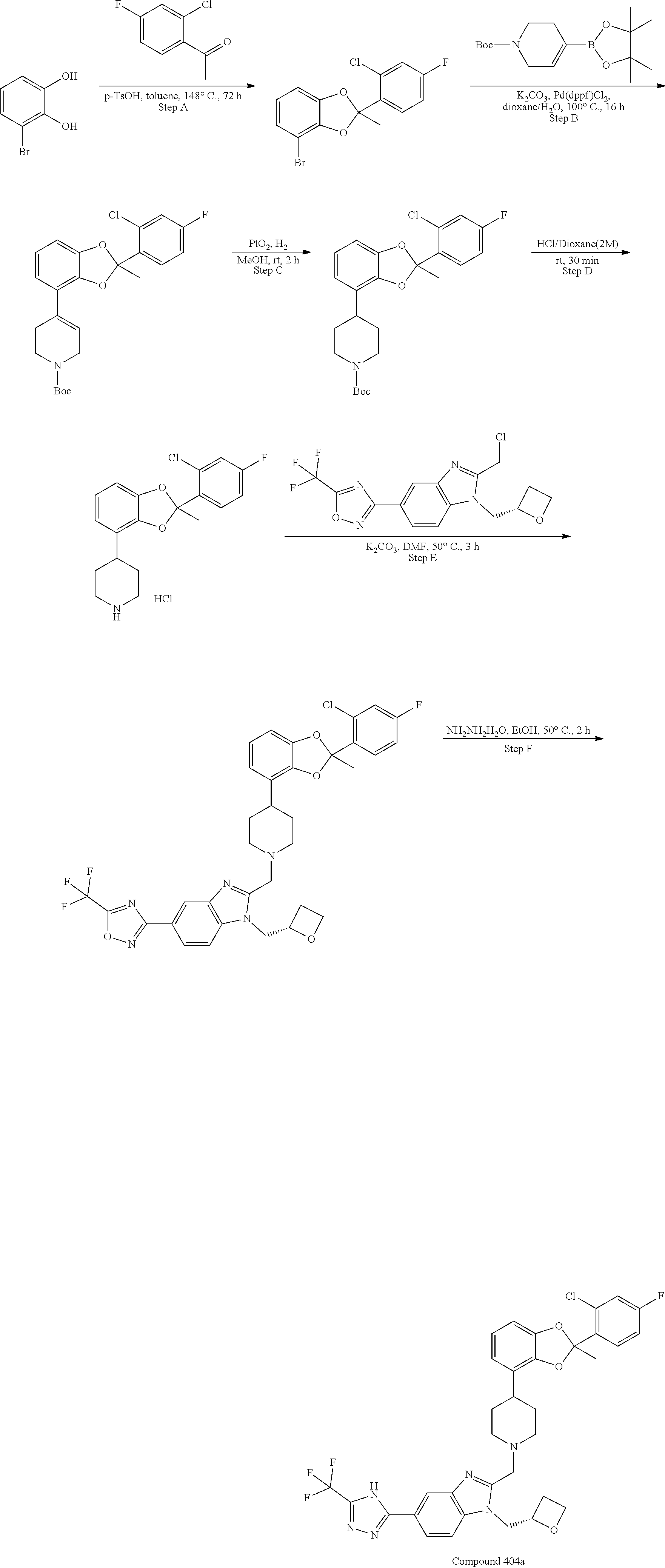

### Step A: 4-bromo-2-(2-chloro-4-fluorophenyl)-2-methylbenzo[d][1,3]dioxole

A mixture of 3-bromobenzene-1,2-diol (5.0 g, 26.4 mmol), 1-(2-chloro-4-fluorophenyl)ethan-1-one (5.0 g, 29.1 mmol) and p-TsOH (251 mg, 1.3 mmol) in toluene (50 mL) was stirred at 148° C. for 72 hours. After the reaction was completed, the mixture was concentrated. The residue was purified by column chromatography (PE: 100%) to give 4-bromo-2-(2-chloro-4-fluorophenyl)-2-methylbenzo[d][1,3]dioxole (3.8 g, yield: 42%) as a colourless oil.

¹H NMR (400 MHz, DMSO-d₆): 7.75 (dd, J=8.8 Hz, 6.4 Hz, 1H), 7.58 (dd, J=8.8 Hz, J=2.4 Hz, 1H), 7.28-7.35 (m, 1H), 7.06 (d, J=8.0 Hz, 1H), 6.99 (d, J=7.6 Hz, 1H), 6.80-6.86 (m, 1H), 2.15 (s, 3H).

### Step B: tert-butyl 4-(2-(2-chloro-4-fluorophenyl)-2-methylbenzo[d][1,3]dioxol-4-yl)-3,6-dihydropyridine-1(2H)-carboxylate

A mixture of 4-bromo-2-(2-chloro-4-fluorophenyl)-2-methylbenzo[d][1,3]dioxole (1.8 g, 5.24 mmol) and tert-butyl 4-(4,4,5,5-tetramethyl-1,3,2-dioxaborolan-2-yl)-3,6-dihydropyridine-1(2H)-carboxylate (1.9 g, 6.3 mmol), K₂CO₃ (2.2 g, 15.7 mmol) and Pd(dppf)Cl₂ (192 mg 0.26 mmol) in Dioxane/H₂O (16 mL/4 mL) was stirred at 100° C. under N₂ (1 atm) for 16 hours. After the reaction was completed, the mixture was diluted with EA (250 mL), washed with H₂O (50 mL×2) and brine (50 mL). The organic layer was dried over anhydrous sodium sulfate, concentrated in vacuum. The residue was purified by column chromatography (PE/EA=20/1) to give tert-butyl 4-(2-(2-chloro-4-fluorophenyl)-2-methylbenzo[d][1,3]dioxol-4-yl)-3,6-dihydropyridine-1(2H)-carboxylate (2.1 g, yield: 91%) as a white solid. MS Calcd.: 445.2; MS Found: 346.2[M+H-Boc]⁺.

### Step C: tert-butyl 4-(2-(2-chloro-4-fluorophenyl)-2-methylbenzo[d][1,3]dioxol-4-yl)piperidine-1-carboxylate

A mixture of tert-butyl 4-(2-(2-chloro-4-fluorophenyl)-2-methylbenzo[d][1,3]dioxol-4-yl)-3,6-dihydropyridine-1(2H)-carboxylate (1.5 g, 3.7 mmol) and PtO₂ (150 mg, 0.66 mmol) in MeOH (15 mL) was stirred at R^{T} under H₂ (1 atm) for 2 hours. After the reaction was completed, the mixture was filtered and concentrated to give tert-butyl 4-(2-(2-chloro-4-fluorophenyl)-2-methylbenzo[d][1,3]dioxol-4-yl)piperidine-1-carboxylate (1.5 g, crude) as white solid. MS Calcd.: 447.2; MS Found: 348.1 [M+H-Boc]⁺.

### Step D: 4-(2-(2-chloro-4-fluorophenyl)-2-methylbenzo[d][1,3]dioxol-4-yl)piperidine

A mixture of tert-butyl 4-(2-(2-chloro-4-fluorophenyl)-2-methylbenzo[d][1,3]dioxol-4-yl)piperidine-1-carboxylate (120 mg) in HCl/Dioxane (2M, 2 mL) was stirred at rt for 30 minutes. After the reaction was completed, the mixture was concentrated in vacuum to give 4-(2-(2-chloro-4-fluorophenyl)-2-methylbenzo[d][1,3]dioxol-4-yl)piperidine hydrogen chloride (103 mg, crude) as a white solid. MS Calcd: 347.1; MS Found: 348.2 [M+H]⁺.

### Step E: 3-(2-((4-(2-(2-chloro-4-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-5-(trifluoromethyl)-1,2,4-oxadiazole

A mixture of 4-(2-(2-chloro-4-fluorophenyl)-2-methylbenzo[d][1,3]dioxol-4-yl)piperidine hydrogen chloride (47 mg) and K₂CO₃ (54 mg, 0.39 mmol) in DMF (1 mL) was stirred at room temperature for 10 minutes. The mixture was added (S)-3-(2-(chloromethyl)-1-(oxetan-2-ylmethyl)-1H-benzo[d]imidazol-5-yl)-5-(trifluoromethyl)-1,2,4-oxadiazole (50 mg, 0.13 mmol) and stirred at 50° C. for 3 hours. After the reaction was completed, the mixture was diluted with EA (20 ml), washed with H₂O (10 ml×3). The organic layer was dried over anhydrous sodium sulfate, filtered, and concentrated in vacuum. The residue was purified by column chromatography (PE/EA=1/1) to give 3-(2-((4-(2-(2-chloro-4-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-5-(trifluoromethyl)-1,2,4-oxadiazole (21 mg, yield: 23%) as a yellow solid. MS Calcd: 683.2; MS Found: 684.4 [M+H]⁺.

### Step F: 2-({4-[2-(2-chloro-4-fluorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 404a)

A mixture of 3-(2-((4-(2-(2-chloro-4-fluorophenyl)-2-methylbenzo[d][1,3]dioxol-4-yl)piperidin-1-yl)methyl)-1-(((S)-oxetan-2-yl)methyl)-1H-benzo[d]imidazol-5-yl)-5-(trifluoromethyl)-1,2,4-oxadiazole (21 mg, 0.03 mmol) and NH₂NH₂·H₂O (4.5 mg, 0.09 mmol) in EtOH (1 mL) was stirred at 50° C. for 2 hours. After the reaction was completed. The reaction mixture was purified by Prep-HPLC to give 2-({4-[2-(2-chloro-4-fluorophenyl)-2-methyl-2H-1,3-benzodioxol-4-yl]piperidin-1-yl}methyl)-1-{[(2S)-oxetan-2-yl]methyl}-5-[5-(trifluoromethyl)-4H-1,2,4-triazol-3-yl]-1H-1,3-benzodiazole (Compound 404a) (8 mg, yield: 39%) as a white solid.

MS Calcd.: 682.2; MS Found: 683.4 [M+H]⁺.

¹H NMR (400 MHz, DMSO-d₆): δ 8.16 (s, 1H), 7.92 (dd, J=8.4 Hz, 1.6 Hz, 1H), 7.75 (dd, J=8.8 Hz, 6.4 Hz, 1H), 7.57-7.63 (m, 1H), 7.54 (dd, J=8.4 Hz, 2.4 Hz, 1H), 7.24-7.30 (m, 1H), 6.72-6.81 (m, 3H), 5.08-5.17 (m, 1H), 4.69-4.77 (m, 1H), 4.56-4.63 (m, 1H), 4.38-4.50 (m, 2H), 3.89-3.96 (m, 1H), 3.73-3.80 (m, 1H), 2.97-3.05 (m, 1H), 2.85-2.93 (m, 1H), 2.57-2.75 (m, 2H), 2.40-2.53 (m, 1H), 2.10-2.30 (m, 2H), 2.08 (d, J=2.0 Hz, 3H), 1.69-1.86 (m, 4H). ¹⁹F-NMR (377 MHz): −60.99, −110.67, −110.70.

### Example 151: 3-chloro-4-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)benzonitrile (Compound 353a)

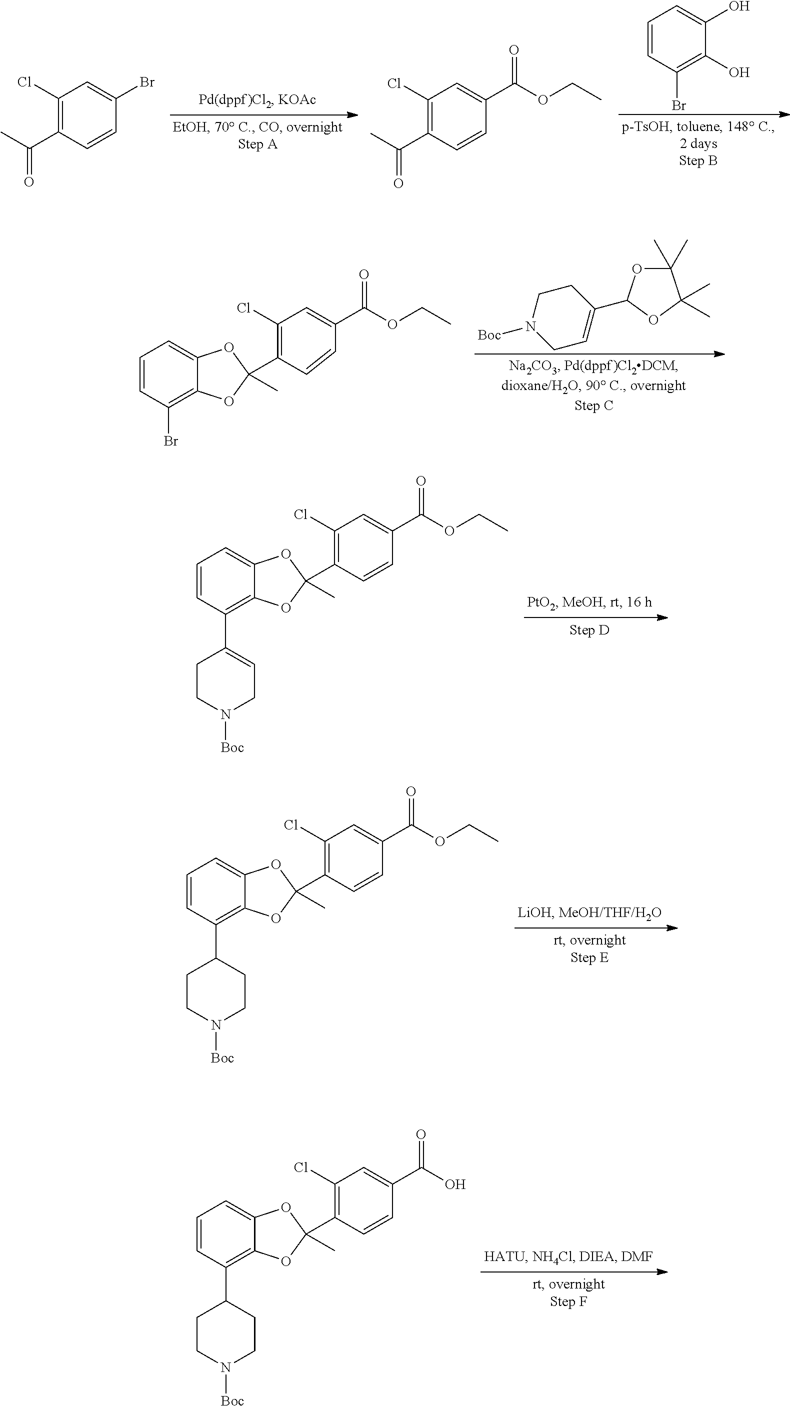

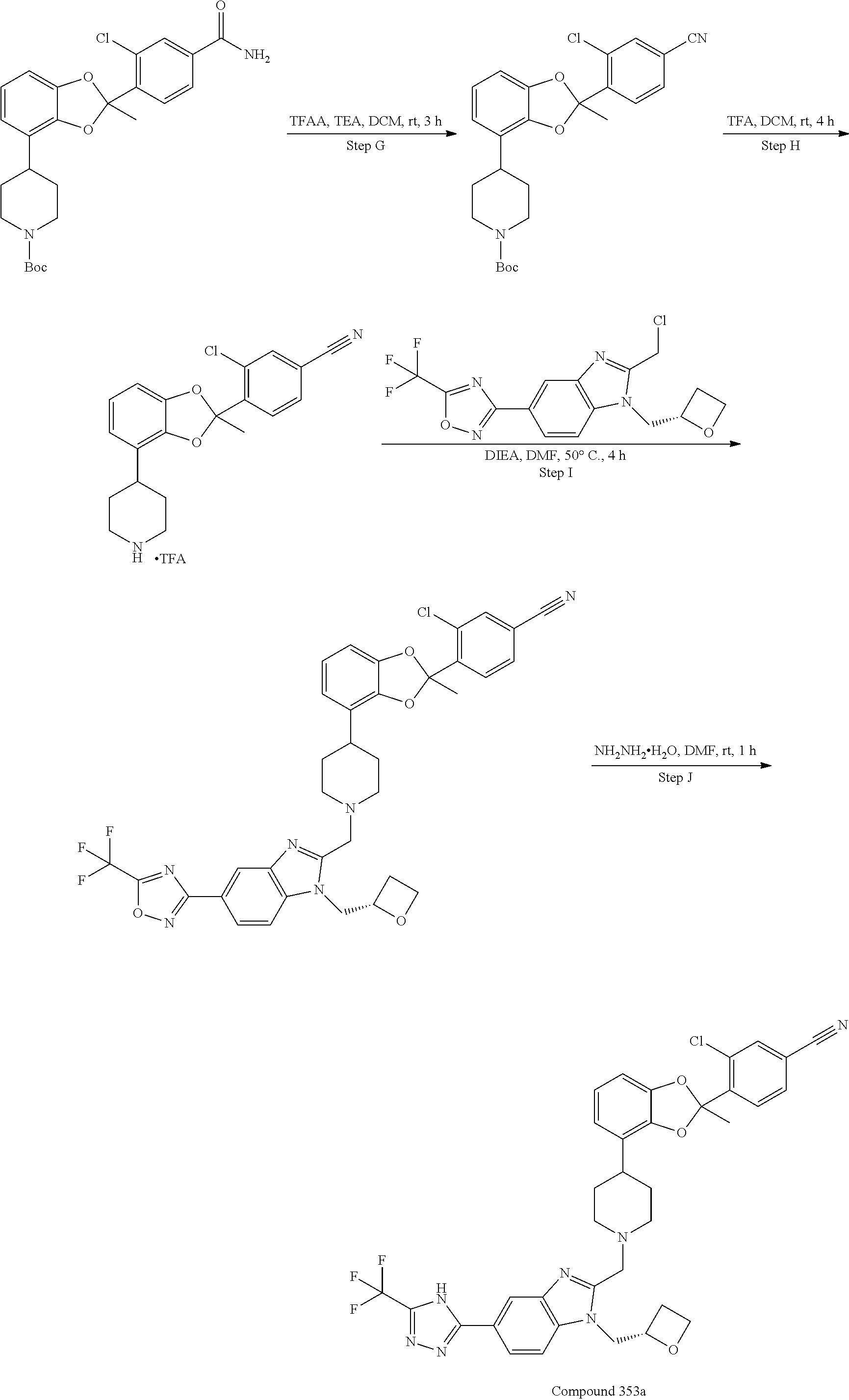

### Step A: ethyl 4-acetyl-3-chlorobenzoate

A mixture of 1-(4-bromo-2-chlorophenyl)ethan-1-one (5.0 g, 21 mmol), KOAc (4.2 g, 43 mmol) and Pd(dppf)Cl₂ (732 mg, 1.0 mmol) in EtOH (100 mL) was stirred at 70° C. under CO overnight. After the reaction was completed, the mixture was diluted with EA (500 mL), washed with H₂O (300 mL). The organic layer was dried over anhydrous sodium sulfate, filtered, and concentrated to dryness. The residue was purified by column chromatography on silica gel (EA/PE=1/10) to give ethyl 4-acetyl-3-chlorobenzoate (4.5 g, yield: 95%) as colorless oil.

¹H NMR (400 MHz, DMSO-d₆): δ 7.96-7.99 (m, 2H), 7.82 (d, J=8.4 Hz, 1H), 4.35 (q, J=7.2 Hz, 2H), 2.61 (s, 3H)), 1.34 (t, J=7.2 Hz, 3H).

### Step B: ethyl 4-(4-bromo-2-methylbenzo[d][1,3]dioxol-2-yl)-3-chlorobenzoate

A mixture of ethyl 4-acetyl-3-chlorobenzoate (2.0 g, 8.8 mmol), 3-bromobenzene-1,2-diol (1.7 g, 8.8 mmol) and p-TsOH (344 mg, 1.8 mmol) in toluene (20 ml) was stirred at 148° C. for 2 days. The reaction was concentrated and purified by column chromatography on silica gel (EA/PE=1/30) to give ethyl 4-(4-bromo-2-methylbenzo[d][1,3]dioxol-2-yl)-3-chlorobenzoate (930 mg, yield: 26%).

¹H NMR (400 MHz, DMSO-d₆): δ 8.06-8.09 (m, 1H), 7.89-7.94 (m, 1H), 7.82 (d, J=8.4 Hz, 1H), 6.95 (dd, J=8.0 Hz, 1.2 Hz, 1H), 6.75-6.78 (m, 1H), 6.66-6.72 (m, 1H), 4.34-4.41 (m, 2H), 2.20 (s, 3H)), 1.38 (t, J=7.2 Hz, 3H).

### Step C: tert-butyl 4-(2-(2-chloro-4-(ethoxycarbonyl)phenyl)-2-methylbenzo[d][1,3]dioxol-4-yl)-3,6-dihydropyridine-1(2H)-carboxylate

A mixture of ethyl 4-(4-bromo-2-methylbenzo[d][1,3]dioxol-2-yl)-3-chlorobenzoate (1.0 g, 2.5 mmol), tert-butyl 4-(4,4,5,5-tetramethyl-1,3-dioxolan-2-yl)-3,6-dihydropyridine-1(2H)-carboxylate (934 mg, 3.0 mmol), Na₂CO₃ (795 mg, 7.5 mmol), Pd(dppf)Cl₂·DCM (102 mg, 0.13 mmol), H₂O (2 mL) in dioxane (20 mL) was stirred at 90° C. overnight under N₂ atmosphere. After the reaction was completed, the mixture was diluted with EA (100 mL), washed with H₂O (60 mL×2). The organic layer was dried over anhydrous sodium sulfate and concentrated in vacuo to dryness. The residue was purified by column chromatography on silica gel (EA/PE=1/20) to give tert-butyl 4-(2-(2-chloro-4-(ethoxycarbonyl)phenyl)-2-methylbenzo[d][1,3]dioxol-4-yl)-3,6-dihydropyridine-1(2H)-carboxylate (1.1 g, yield: 88%). MS Calcd.: 499.2; MS Found: 522.2 [M+Na]⁺.

### Step D: tert-butyl 4-(2-(2-chloro-4-(ethoxycarbonyl)phenyl)-2-methylbenzo[d][1,3]dioxol-4-yl)piperidine-1-carboxylate

A mixture of tert-butyl 4-(2-(2-chloro-4-(ethoxycarbonyl)phenyl)-2-methylbenzo[d][1,3]dioxol-4-yl)-3,6-dihydropyridine-1(2H)-carboxylate (1.1 g, 2.2 mmol), PtO₂ (100 mg, 0.44 mmol) in MeOH (20 ml) was stirred at room temperature under H₂ for 16 hours. After the reaction was completed, the mixture was filtered, the filtrate was concentrated in vacuo to dryness. The residue was purified by column chromatography on silica gel (EA/PE=1/20) to give tert-butyl 4-(2-(2-chloro-4-(ethoxycarbonyl)phenyl)-2-methylbenzo[d][1,3]dioxol-4-yl)piperidine-1-carboxylate (820 mg, yield: 74%). MS Calcd.: 501.2; MS Found: 402.1 [M-Boc+H]⁺.

### Step E: 4-(4-(1-(tert-butoxycarbonyl)piperidin-4-yl)-2-methylbenzo[d][1,3]dioxol-2-yl)-3-chlorobenzoic acid

A mixture of tert-butyl 4-(2-(2-chloro-4-(ethoxycarbonyl)phenyl)-2-methylbenzo[d][1,3]dioxol-4-yl)piperidine-1-carboxylate (820 mg, 1.6 mmol), LiOH H₂O (137 mg, 3.2 mmol), THE (5 mL), H₂O (5 mL) in MeOH (10 ml) was stirred at room temperature overnight. After the reaction was completed, the mixture was diluted with water (30 mL), the pH value was adjusted to 6 with 1N HCl, then extracted with EA (100 mL*2). The combined organic layers were dried over anhydrous sodium sulfate, filtered and the filtrate was concentrated to give 4-(4-(1-(tert-butoxycarbonyl)piperidin-4-yl)-2-methylbenzo[d][1,3]dioxol-2-yl)-3-chlorobenzoic acid (720 mg, yield: 95%). MS Calcd.: 473.2; MS Found: 496.1 [M+Na]⁺.

### Step F: tert-butyl 4-(2-(4-carbamoyl-2-chlorophenyl)-2-methylbenzo[d][1,3]dioxol-4-yl)piperidine-1-carboxylate

A mixture of 4-(4-(1-(tert-butoxycarbonyl)piperidin-4-yl)-2-methylbenzo[d][1,3]dioxol-2-yl)-3-chlorobenzoic acid (720 mg, 1.5 mmol), NH₄Cl (814 mg, 15.2 mmol), HATU (1.1 g, 3.0 mmol), DIEA (3.9 g, 30.3 mmol) in DMF (20 ml) was stirred at room temperature overnight. After the reaction was completed, the mixture was diluted with EA (100 ml), washed with H₂O (80 ml×3). The organic layer was dried over anhydrous sodium sulfate and concentrated in vacuo to dryness.

The residue was purified by column chromatography on silica gel (EA/PE=2/3) to give tert-butyl 4-(2-(4-carbamoyl-2-chlorophenyl)-2-methylbenzo[d][1,3]dioxol-4-yl)piperidine-1-carboxylate (550 mg, yield: 76%). MS Calcd.: 472.2; MS Found: 495.1 [M+Na]⁺.

### Step G: tert-butyl 4-(2-(2-chloro-4-cyanophenyl)-2-methylbenzo[d][1,3]dioxol-4-yl)piperidine-1-carboxylate

A mixture of tert-butyl 4-(2-(4-carbamoyl-2-chlorophenyl)-2-methylbenzo[d][1,3]dioxol-4-yl)piperidine-1-carboxylate (550 mg, 1.2 mmol), TEA (210 mg, 2.4 mmol), trifluoroacetic anhydride (320 mg, 1.8 mmol) in DCM (10 ml) was stirred at rt for 3 hours. After the reaction was completed, the mixture was diluted with EA (50 mL), washed with saturated aqueous NaHCO₃. The organic layer was dried over anhydrous sodium sulfate and concentrated in vacuo to dryness. The residue was purified by column chromatography on silica gel (EA/PE=1/10) to give tert-butyl 4-(2-(2-chloro-4-cyanophenyl)-2-methylbenzo[d][1,3]dioxol-4-yl)piperidine-1-carboxylate (369 mg, yield: 67%). MS Calcd.: 454.2; MS Found: 355.1 [M-Boc+H]⁺.

### Step H: 3-chloro-4-(2-methyl-4-(piperidin-4-yl)benzo[d][1,3]dioxol-2-yl)benzonitrile. TFA salt

A mixture of tert-butyl 4-(2-(2-chloro-4-cyanophenyl)-2-methylbenzo[d][1,3]dioxol-4-yl)piperidine-1-carboxylate (122 mg, 0.27 mmol) and TFA (1 mL) in DCM (4 mL) was stirred at rt for 4 hours. After the reaction was completed, the mixture was concentrated to give 3-chloro-4-(2-methyl-4-(piperidin-4-yl)benzo[d][1,3]dioxol-2-yl)benzonitrile TFA salt (201 mg, crude). MS Calcd.: 354.1; MS Found: 355.1 [M+H]⁺.

### Step I: 3-chloro-4-(2-methyl-4-(1-((1-(((S)-oxetan-2-yl)methyl)-5-(5-(trifluoromethyl)-1,2,4-oxadiazol-3-yl)-1H-benzo[d]imidazol-2-yl)methyl)piperidin-4-yl)benzo[d][1,3]dioxol-2-yl)benzonitrile

A mixture of 3-chloro-4-(2-methyl-4-(piperidin-4-yl)benzo[d][1,3]dioxol-2-yl)benzonitrile. TFA salt (201 mg, crude), (S)-3-(2-(chloromethyl)-1-(oxetan-2-ylmethyl)-1H-benzo[d]imidazol-5-yl)-5-(trifluoromethyl)-1,2,4-oxadiazole (100 mg, 0.27 mmol), DIEA (174 mg, 1.35 mmol) in DMF (2 mL) was stirred at 50° C. for 4 hours. After the reaction was completed, the mixture was diluted with EA (20 mL), washed with H₂O (10 mL×3). The organic layer was dried over anhydrous sodium sulfate and concentrated in vacuo to dryness. The residue was purified by column chromatography on silica gel (EA/PE=3/1) to give 3-chloro-4-(2-methyl-4-(1-((1-(((S)-oxetan-2-yl)methyl)-5-(5-(trifluoromethyl)-1,2,4-oxadiazol-3-yl)-1H-benzo[d]imidazol-2-yl)methyl)piperidin-4-yl)benzo[d][1,3]dioxol-2-yl)benzonitrile (120 mg, yield: 64%). MS Calcd.: 690.2; MS Found: 691.2 [M+H]⁺.

### Step J: 3-chloro-4-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)benzonitrile (Compound 353a)

A mixture of 3-chloro-4-(2-methyl-4-(1-((1-(((S)-oxetan-2-yl)methyl)-5-(5-(trifluoromethyl)-1,2,4-oxadiazol-3-yl)-1H-benzo[d]imidazol-2-yl)methyl)piperidin-4-yl)benzo[d][1,3]dioxol-2-yl)benzonitrile (70 mg, 0.10 mmol) and hydrazine hydrate (6 mg, 0.12 mmol) in DMF (1 mL) was stirred at rt for 1 hour. The mixture was diluted with EA (20 mL), washed with H₂O (10 mL×3). The organic layer was dried over anhydrous sodium sulfate and concentrated in vacuo to dryness. The residue was purified by prep-HPLC to give 3-chloro-4-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)benzonitrile (Compound 353a) (4 mg, yield: 6%) as a white solid.

MS Calcd.: 689.2; MS Found: 690.4 [M+H]⁺.

¹H NMR (400 MHz, DMSO-d₆): δ 8.27 (s. 1H), 8.15 (s, 1H), 7.82-7.95 (m, 4H), 6.73-6.86 (m, 3H), 5.08-5.20 (m. 1H), 4.76-4.85 (m. 1H), 4.62-4.70 (m, 1H), 4.38-4.53 (m. 2H), 3.92-4.01 (m, 1H), 3.78-3.84 (m, 1H), 2.98-3.07 (m. 1H), 2.85-2.92 (m. 1H), 2.60-2.78 (m. 2H), 2.40-2.52 (m, 1H), 2.12-2.35 (m. 2H), 2.11 (d, J=2.4 Hz, 3H), 1.68-1.87 (m, 4H). ¹⁹F-NMR (377 MHz): −63.77.

### Example 152: 6-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)pyridine-3-carbonitrile (Compound 354a)

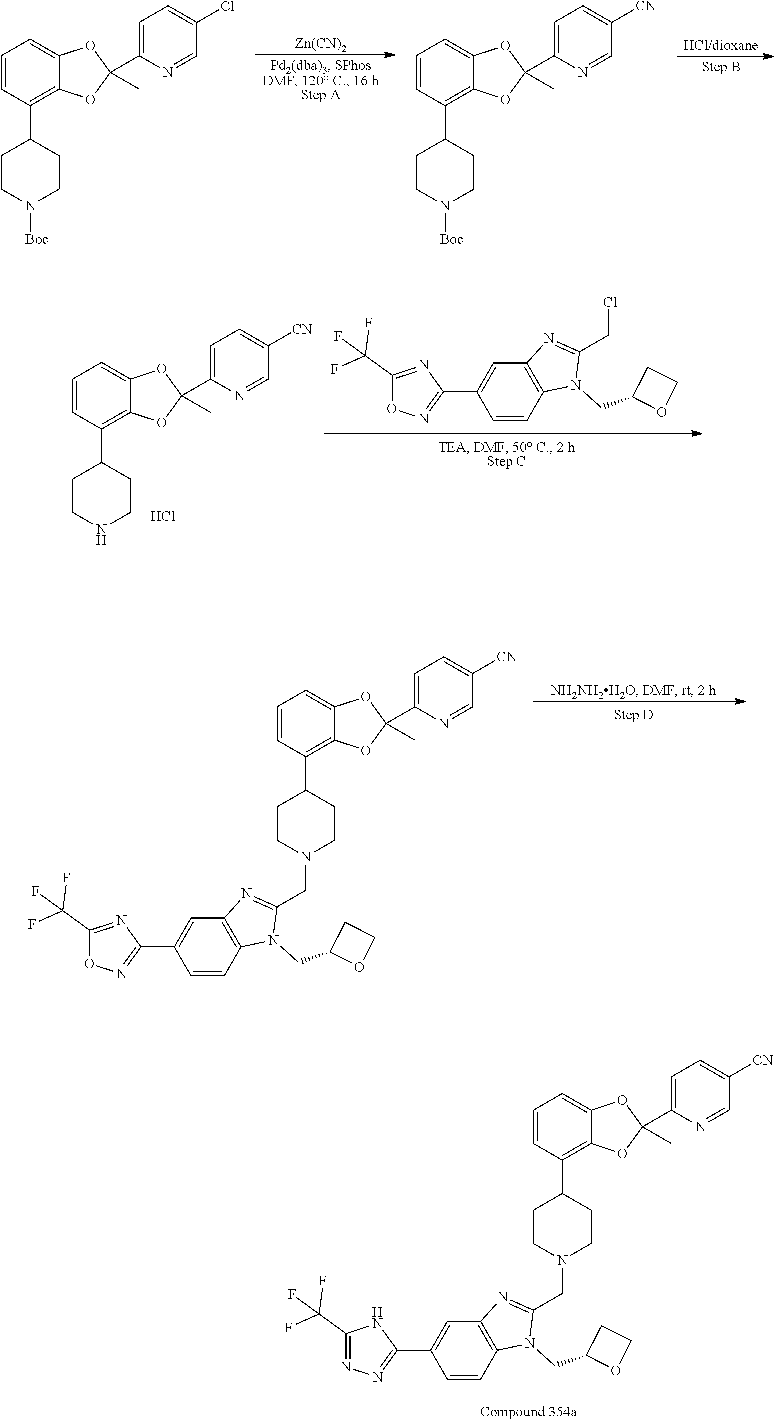

### Step A: tert-butyl 4-(2-(5-cyanopyridin-2-yl)-2-methylbenzo[d][1,3]dioxol-4-yl)piperidine-1-carboxylate

A mixture of tert-butyl 4-(2-(5-chloropyridin-2-yl)-2-methylbenzo[d][1,3]dioxol-4-yl)piperidine-1-carboxylate (300 mg, 0.7 mmol), Zn(CN)₂ (81 mg, 1.4 mmol), Pd₂(dba)₃ (32 mg, 0.07 mmol) and SPhos (15 mg, 0.07 mmol) in DMF (3 mL) was degassed and charged with N₂. The reaction was stirred at 120° C. for 16 hours. After the reaction was completed, the reaction was quenched with water (30 mL) and extracted with ethyl acetate (50 mL×3). The organic layer was combined and washed with brine (50 mL×2), dried over sodium sulfate, filtered, and concentrated in vacuum. The residue was purified by column chromatography on silica gel (PE:EA=3:1) to give tert-butyl 4-(2-(5-cyanopyridin-2-yl)-2-methylbenzo[d][1,3]dioxol-4-yl)piperidine-1-carboxylate (200 mg, 68% yield) as a yellow solid. MS Calcd.: 421.2; MS Found: 322.1 [M-100+H]⁺.

### Step B: 6-(2-methyl-4-(piperidin-4-yl)benzo[d][1,3]dioxol-2-yl)nicotinonitrile HCl salt

To a solution of tert-butyl 4-(2-(5-cyanopyridin-2-yl)-2-methylbenzo[d][1,3]dioxol-4-yl)piperidine-1-carboxylate (200 mg, 0.50 mmol) in dioxane (2 mL) was added HCl/dioxane (4M, 2 mL). The reaction was stirred at room temperature for 1 hour. After the reaction was completed, the reaction was concentrated in vacuum to give 6-(2-methyl-4-(piperidin-4-yl)benzo[d][1,3]dioxol-2-yl)nicotinonitrile HCl salt (130 mg, crude) as a yellow solid. MS Calcd.: 321.2; MS Found: 322.1 [M+H]⁺.

### Step C: 6-(2-methyl-4(1-((1-(((S)-oxetan-2-yl)methyl)-5-(5-(trifluoromethyl)-1,2,4-oxadiazol-3-yl)-1H-benzo[d]imidazol-2-yl)methyl)piperidin-4-yl)benzo[d][1,3]dioxol-2-yl)nicotinonitrile

A mixture of (S)-3-(2-(chloromethyl)-1-(oxetan-2-ylmethyl)-1H-benzo[d]imidazol-5-yl)-5-(trifluoromethyl)-1,2,4-oxadiazole (112 mg, 0.3 mmol) and 6-(2-methyl-4-(piperidin-4-yl)benzo[d][1,3]dioxol-2-yl)nicotinonitrile HCl salt (100 mg) and TEA (91 mg, 0.9 mmol) in DMF (3 mL) was stirred at 50° C. for 2 hours. After the reaction was completed, the reaction was quenched with water (30 mL) and extracted with ethyl acetate (50 mL×3). The organic layer was combined and washed with brine (50 mL×2), dried over sodium sulfate, filtered and concentrated in vacuum to give 6-(2-methyl-4-(1-((1-(((S)-oxetan-2-yl)methyl)-5-(5-(trifluoromethyl)-1,2,4-oxadiazol-3-yl)-1H-benzo[d]imidazol-2-yl)methyl)piperidin-4-yl)benzo[d][1,3]dioxol-2-yl)nicotinonitrile (230 mg, crude) as yellow oil. MS Calcd.: 657.2; MS Found: 658.3 [M+H]⁺.

### Step D: 6-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)pyridine-3-carbonitrile (Compound 354a)

To a solution of 6-(2-methyl-4-(1-((1-(((S)-oxetan-2-yl)methyl)-5-(5-(trifluoromethyl)-1,2,4-oxadiazol-3-yl)-1H-benzo[d]imidazol-2-yl)methyl)piperidin-4-yl)benzo[d][1,3]dioxol-2-yl)nicotinonitrile (50 mg, 0.07 mmol) in DMF (1 mL) was added NH₂NH₂·H₂O (7.6 mg, 0.14 mmol). The reaction was stirred at room temperature for 2 hours. After the reaction was completed, the reaction mixture was purified directly by Prep-HPLC to give 6-(2-methyl-4-{1-[(1-{[(2S)-oxetan-2-yl]methyl}-5-[5-(trifluoromethyl)-4H-1,2,4-triazol-3-yl]-1H-1,3-benzodiazol-2-yl)methyl]piperidin-4-yl}-2H-1,3-benzodioxol-2-yl)pyridine-3-carbonitrile Compound 354a (5.84 mg, 12% yield) as a white solid. MS Calcd.: 656.2; MS Found: 657.5 [M+H]⁺.

¹H NMR (400 MHz, DMSO-d₆): δ 9.14 (d, J=1.2 Hz, 1H), 8.42 (dd, J=8.0 Hz, J=2.0 Hz, 1H), 8.39 (d, J=0.8 Hz, 1H), 8.03 (dd, J=8.8 Hz, J=1.6 Hz, 1H), 7.96 (d, J=8.4 Hz, 1H), 7.80-7.83 (m, 1H), 6.88 (d, J=4.4 Hz, 2H), 6.75-6.82 (m, 1H), 5.02-5.11 (m, 1H), 4.75-4.85 (m, 1H), 4.60-4.68 (m, 1H), 4.47-4.53 (m, 1H), 4.32-4.39 (m, 1H), 3.70-3.82 (m, 2H), 3.27-3.40 (m, 2H), 2.85-3.10 (m, 1H), 2.65-2.78 (m, 1H), 2.53-2.58 (m, 1H), 2.30-2.40 (m, 1H), 1.98-2.15 (m, 8H).

### Example 153: 2-{[4-(2-methyl-2-phenyl-2H-1,3-benzodioxol-4-yl)piperidin-1-yl]methyl}-1-{[(2S)-oxetan-2-yl]methyl}-5-[5-(trifluoromethyl)-4H-1,2,4-triazol-3-yl]-1H-1,3-benzodiazole (Compound 405a)

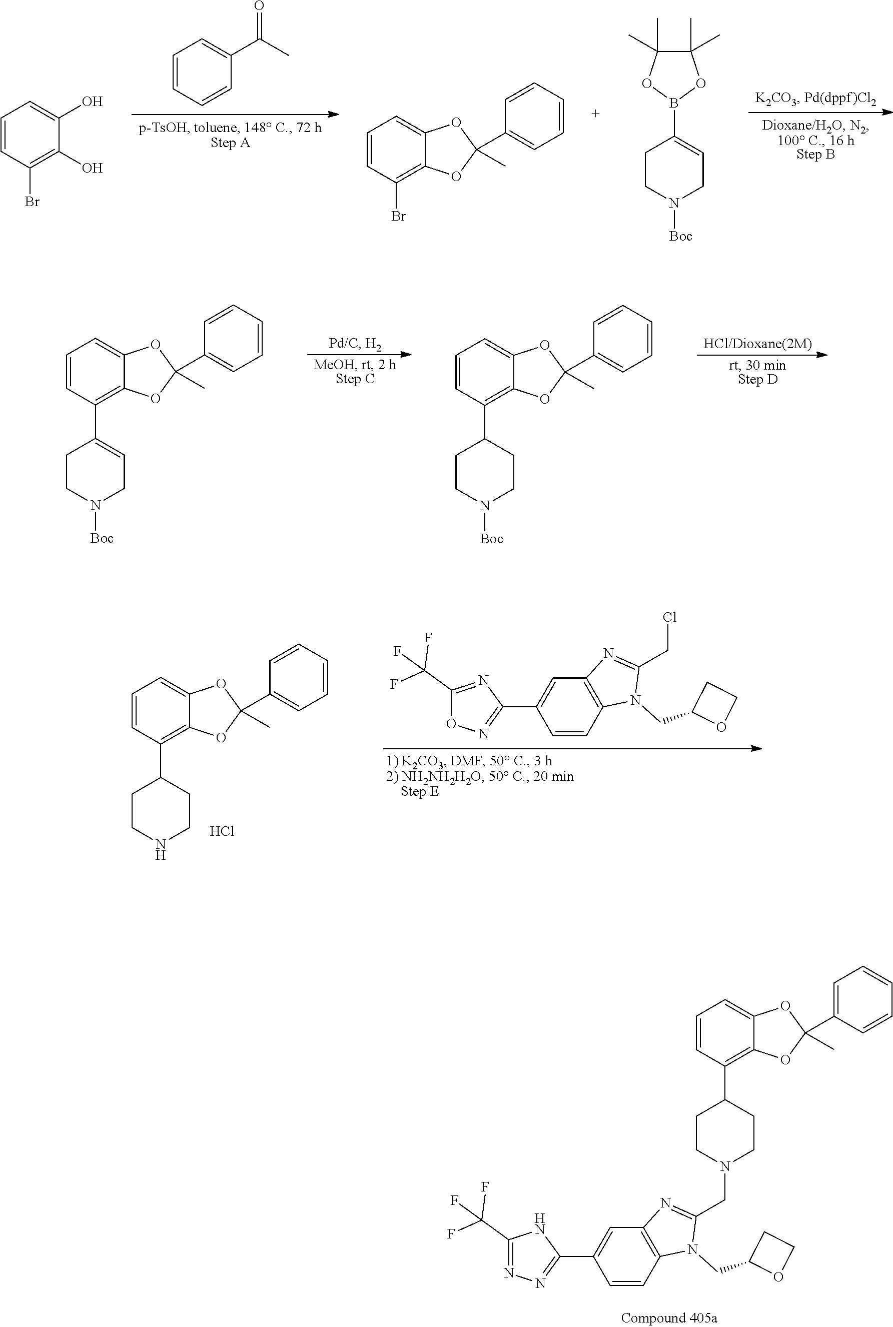

### Step A: 4-bromo-2-methyl-2-phenylbenzo[d][1,3]dioxole

A mixture of 3-bromobenzene-1,2-diol (5.0 g, 26.4 mmol) and acetophenone (3.5 g, 29.1 mmol), p-TsOH (251 mg, 1.3 mmol) in toluene (50 mL) was stirred at 148° C. for 72 hours. After the reaction was completed, the mixture was concentrated in vacuum. The residue was purified by column chromatography (PE=100%) to give 4-bromo-2-methyl-2-phenylbenzo[d][1,3]dioxole (650 mg, yield: 8%) as colorless oil.

¹H NMR (400 MHz, DMSO-d₆): 7.57-7.61 (m, 2H), 7.40-7.52 (m, 3H), 7.02 (d, J=8.4 Hz, 1H), 6.95 (d, J=6.8 Hz, 1H), 6.80 (t, J=8.4 Hz, 1H), 2.04 (s, 3H).

### Step B: tert-butyl 4-(2-methyl-2-phenylbenzo[d][1,3]dioxol-4-yl)-3,6-dihydropyridine-1(2H)-carboxylate

A mixture of 4-bromo-2-methyl-2-phenylbenzo[d][1,3]dioxole (650 mg, 2.3 mmol) and tert-butyl 4-(4,4,5,5-tetramethyl-1,3,2-dioxaborolan-2-yl)-3,6-dihydropyridine-1(2H)-carboxylate (828 mg, 2.68 mmol), K₂CO₃ (923 mg, 6.7 mmol), Pd(dppf)Cl₂ (82 mg, 0.11 mmol) in Dioxane/H₂O (7 mL/3 mL) was stirred at 100° C. under N₂ (1 atm) for 16 hour. After the reaction was completed, the mixture was filtered, and the filtrate was concentrated in vacuum. The residue was purified by column chromatography (PE:EA=25:1) to give tert-butyl 4-(2-methyl-2-phenylbenzo[d][1,3]dioxol-4-yl)-3,6-dihydropyridine-1(2H)-carboxylate (760 mg, yield: 86%) as yellow oil. MS Calcd: 393.2; MS Found: 338.1[M+H-56]⁺.

### Step C: tert-butyl 4-(2-methyl-2-phenylbenzo[d][1,3]dioxol-4-yl)piperidine-1-carboxylate

A mixture of tert-butyl 4-(2-methyl-2-phenylbenzo[d][1,3]dioxol-4-yl)-3,6-dihydropyridine-1(2H)-carboxylate (760 mg, 1.93 mmol) and Pd/C (76 mg, 10% on Carbon, wetted with ca. 55% water) in MeOH (8 mL) was stirred at RT under H₂ (1 atm) for 2 hours. After the reaction was completed, the mixture was filtered and the filtrate concentrated in vacuum to give tert-butyl 4-(2-methyl-2-phenylbenzo[d][1,3]dioxol-4-yl)piperidine-1-carboxylate (700 mg, crude) as yellow oil. MS Calcd: 395.2; MS Found: 340.1[M+H-56]⁺.

### Step D: 4-(2-methyl-2-phenylbenzo[d][1,3]dioxol-4-yl)piperidine hydrochloride

A mixture of tert-butyl 4-(2-methyl-2-phenylbenzo[d][1,3]dioxol-4-yl)piperidine-1-carboxylate (53 mg, crude) in HCl/Dioxane (2M, 2 mL) was stirred at RT for 30 min. After the reaction was completed, the mixture was concentrated in vacuum to give 4-(2-methyl-2-phenylbenzo[d][1,3]dioxol-4-yl)piperidine hydrochloride (43 mg, crude) as a white solid. MS Calcd: 295.2; MS Found: 296.3 [M+H]⁺.

### Step E: 2-{[4-(2-methyl-2-phenyl-2H-1,3-benzodioxol-4-yl)piperidin-1-yl]methyl}-1-{[(2S)-oxetan-2-yl]methyl}-5-[5-(trifluoromethyl)-4H-1,2,4-triazol-3-yl]-1H-1,3-benzodiazole (Compound 405a)

A mixture of 4-(2-methyl-2-phenylbenzo[d][1,3]dioxol-4-yl)piperidine hydrochloride (43 mg, 0.13 mmol) and K₂CO₃ (54 mg, 0.39 mmol) in DMF (1 mL) was stirred at room temperature for 10 minutes. (S)-3-(2-(chloromethyl)-1-(oxetan-2-ylmethyl)-1H-benzo[d]imidazol-5-yl)-5-(trifluoromethyl)-1,2,4-oxadiazole (50 mg, 0.13 mmol) was added and the mixture was stirred at 50° C. for 3 hours. The mixture was added NH₂NH₂·H₂O (0.02 ml, 0.39 mmol) and stirred at 50° C. for 20 min. After the reaction was completed, the reaction mixture was purified directly by Prep-HPLC to give 2-{[4-(2-methyl-2-phenyl-2H-1,3-benzodioxol-4-yl)piperidin-1-yl]methyl}-1-{[(2S)-oxetan-2-yl]methyl}-5-[5-(trifluoromethyl)-4H-1,2,4-triazol-3-yl]-1H-1,3-benzodiazole (Compound 405a) (12 mg, yield: 15%) as a white solid.

MS Calcd: 630.3; MS Found: 631.4 [M+H]⁺.

¹H NMR (400 MHz, DMSO-d₆): δ 8.20 (s, 1H), 7.92 (d, J=8.0 Hz, 1H), 7.70 (d, J=8.4 Hz, 1H), 7.56-7.60 (m, 2H), 7.35-7.45 (m, 3H), 6.70-6.78 (m, 3H), 5.10-5.20 (m, 1H), 4.71-4.79 (m, 1H), 4.57-4.64 (m, 1H), 4.38-4.51 (m, 2H), 3.94 (d, J=13.2 Hz, 1H), 3.78 (d, J=13.6 Hz, 1H), 2.99-3.07 (m, 1H), 2.85-2.93 (m, 1H), 2.60-2.75 (m, 2H), 2.40-2.53 (m, 1H), 2.14-2.31 (m, 2H), 1.97 (s, 3H), 1.68-1.83 (m, 4H).

### Example 154: 3-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-4,5-dihydro-1H-1,2,4-triazol-5-one (Compound 355a)

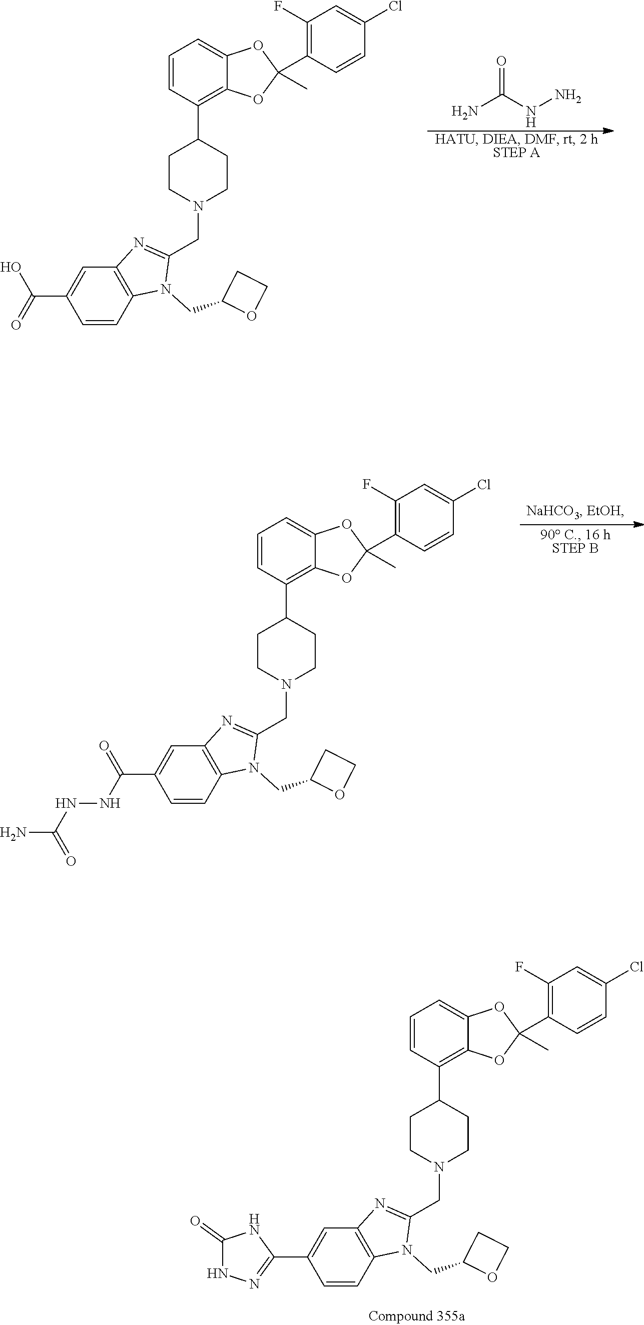

### Step A: 2-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonyl)hydrazine-1-carboxamide

To a solution of hydrazinecarboxamide (86.35 mg, 1.1 mmol) in DMF (2 mL) was added DIEA (155 mg, 1.1 mmol), HATU (200 mg, 0.55 mmol) and 2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carboxylic acid (250 mg, 0.42 mmol). The resulting mixture was stirred at room temperature for 2 h. After the reaction was completed, the reaction was quenched with water (30 mL) and extracted with DCM (30 mL×3). The organic layer was combined and washed with brine (30 mL×2), dried over sodium sulfate, filtered and concentrated in vacuum to give 2-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonyl)hydrazine-1-carboxamide (230 mg, yield: 73%) as a white solid. MS Calcd.: 648.2; MS Found: 649.3 [M+H]⁺.

### Step B: 3-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-4,5-dihydro-1H-1,2,4-triazol-5-one (Compound 355a)

A mixture of 2-(2-((4-(2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-5-carbonyl)hydrazine-1-carboxamide (150 mg, 0.23 mmol), NaHCO₃ (85 mg, 1.01 mmol) in EtOH (2 mL) and H₂O (0.5 mL) was stirred at 90° C. for 16 hours. The reaction mixture was diluted with water (10 mL), the pH value was adjusted to 6 with con. HCl, extracted with EA (20 mL×3). The organic layers were combined and concentrated under vacuum. The residue was purified by Prep-HPLC to give 3-[2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazol-5-yl]-4,5-dihydro-1H-1,2,4-triazol-5-one (Compound 355a) (0.86 mg, yield: 0.6%) as a white solid. MS Calcd.: 630.2; MS Found: 631.3 [M+H]⁺.

¹H NMR (400 MHz, DMSO-d₆): δ 8.30-8.50 (m, 2H), 8.07 (s, 1H), 7.52-7.68 (m, 3H), 7.30-7.38 (m, 1H), 6.70-6.80 (m, 3H), 5.10-5.20 (m, 1H), 4.69-4.77 (m, 1H), 4.55-4.65 (m, 1H), 4.40-4.52 (m, 2H), 3.94 (d, J=14.0 Hz, 1H), 3.77 (d, J=13.6 Hz, 1H), 2.97-3.05 (m, 1H), 2.83-2.91 (m, 1H), 2.65-2.74 (m, 3H), 2.10-2.30 (m, 2H), 2.02 (s, 3H), 1.69-1.80 (m, 4H). ¹⁹F-NMR (377 MHz): −110.83.

### Example 155: 2-({4-[(2S)-2-(4-chloro-2-fluorophenyl)-2-methyl-2H-1,3-benzodioxol-4-yl]piperidin-1-yl}methyl)-5-(5-methyl-4H-1,2,4-triazol-3-yl)-1-{[(2S)-oxetan-2-yl]methyl}-1H-1,3-benzodiazole (Compound 406a)

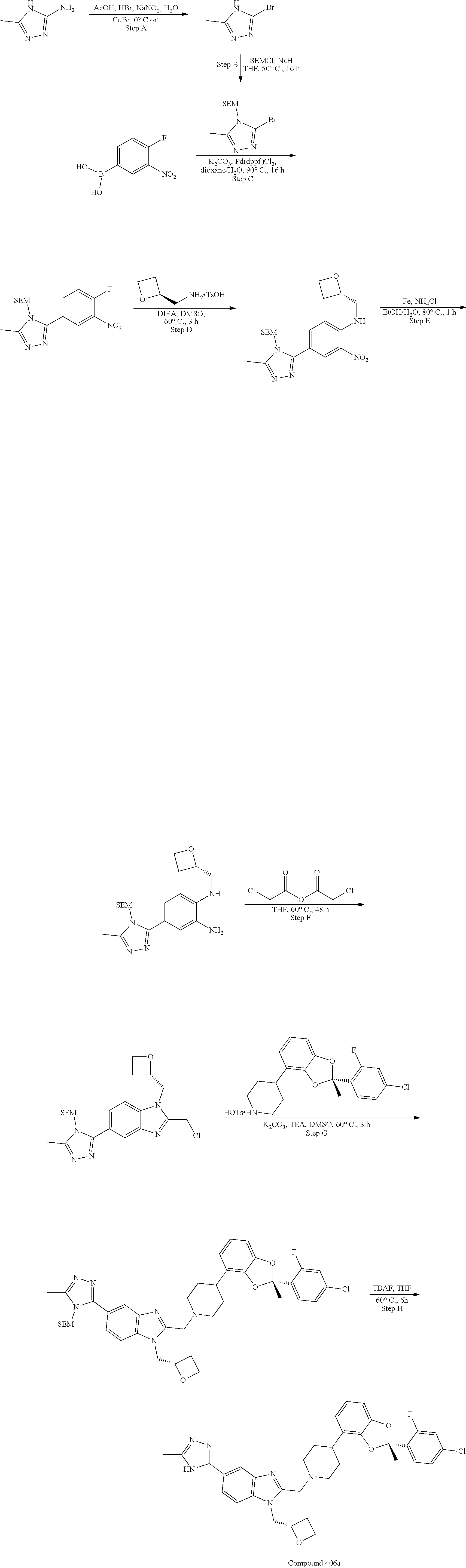

### Step A: 3-bromo-5-methyl-4H-1,2,4-triazole

To a solution of 5-methyl-4H-1,2,4-triazol-3-amine (20.0 g, 205 mmol) in AcOH (130 mL) was added HBr (40% aqueous solution, 70 mL), NaNO₂ (13.0 g, 94.2 mmol) in H₂O (40 mL) at 0° C. The mixture was stirred at 0° C. for 10 mins, CuBr (50 g, 340 mmol) in HBr (40% aqueous solution, 90 mL) was added to the solution dropwise at 0° C. The reaction mixture was stirred at rt for 1 hour. The mixture was quenched with aqueous NaHCO₃ and extracted with EtOAc. The organic phase was dried over Na₂SO₄, filtered, and concentrated in vacuum to give 3-bromo-5-methyl-4H-1,2,4-triazole (2.4 g) as a white solid. MS Calcd.: 161.0; MS Found: 162.1 [M+H]⁺.

### Step B: 3-bromo-5-methyl-4-((2-(trimethylsilyl)ethoxy)methyl)-4H-1,2,4-triazole

To a solution of SEMCl (4.9 g, 29.5 mmol) in dry THF (150 mL) was added NaH (1.2 g, 60% purity, 29.5 mmol) at room temperature and the mixture was stirred at 50° C. for 30 mins, 3-bromo-5-methyl-4H-1,2,4-triazole (2.4 g) was added and the mixture was stirred at 50° C. for another 16 hours. The mixture was quenched with water and extracted with EtOAc, the organic phase was washed by brine, dried over Na₂SO₄, filtered and concentrated in vacuum and purified by pre-HPLC to give compound 3-bromo-5-methyl-4-((2-(trimethylsilyl)ethoxy)methyl)-4H-1,2,4-triazole (1.2 g) as a light-yellow oil. MS Calcd.: 291.0; MS Found: 292.1 [M+H]⁺.

NOTE: The product might be a mixture of the following three structures. We choose b as the representative for subsequent reaction:

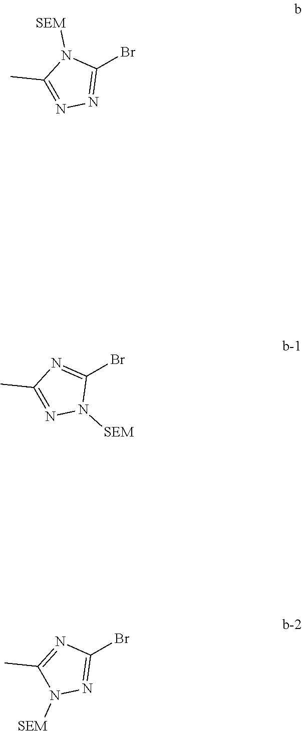

### Step C: 3-(4-fluoro-3-nitrophenyl)-5-methyl-4-((2-(trimethylsilyl)ethoxy)methyl)-4H-1,2,4-triazole

A mixture of (4-fluoro-3-nitrophenyl)boronic acid (500 mg, 2.7 mmol) and 3-bromo-5-methyl-4-((2-(trimethylsilyl)ethoxy)methyl)-4H-1,2,4-triazole (786 mg, 2.7 mmol) in dioxane (8 mL) and H₂O (2 mL) were added K₂CO₃ (1.49 g, 10.8 mmol), Pd(dppf)Cl₂ (198 mg, 0.27 mmol). The reaction mixture was stirred at 90° C. under N₂ for 16 hours. The mixture was poured into cold water (50 mL) and extracted with EtOAc (2×50 mL), the combined organic layers were washed with brine, dried over sodium sulfate, filtered and the residue was purified by column chromatography to furnish 3-(4-fluoro-3-nitrophenyl)-5-methyl-4-((2-(trimethylsilyl)ethoxy)methyl)-4H-1,2,4-triazole (600 mg, yield: 63%) as yellow oil. MS Calcd.: 352.1; MS Found: 353.1 [M+H]⁺.

### Step D: (S)-4-(5-methyl-4-((2-(trimethylsilyl)ethoxy)methyl)-4H-1,2,4-triazol-3-yl)-2-nitro-N-(oxetan-2-ylmethyl)aniline

A mixture of 3-(4-fluoro-3-nitrophenyl)-5-methyl-4-((2-(trimethylsilyl)ethoxy)methyl)-4H-1,2,4-triazole (600 mg, 1.7 mmol), (S)-oxetan-2-ylmethanamine 4-methylbenzenesulfonate (486 mg, 1.9 mmol) in DMSO (8 mL) were added DIEA (2.2 g, 17 mmol). The reaction mixture was stirred at 60° C. for 3 hours. The mixture was poured into cold water (30 mL) and extracted with EtOAc (2×30 mL), the combined organic layers were washed with brine, dried over sodium sulfate, filtered and concentrated under reduced pressure. The residue was purified by column chromatography to furnish (S)-4-(5-methyl-4-((2-(trimethylsilyl)ethoxy)methyl)-4H-1,2,4-triazol-3-yl)-2-nitro-N-(oxetan-2-ylmethyl)aniline (600 mg, yield: 84%) as yellow solid. MS Calcd.: 419.2; MS Found: 420.2 [M+H]⁺.

### Step E: (S)-4-(5-methyl-4-((2-(trimethylsilyl)ethoxy)methyl)-4H-1,2,4-triazol-3-yl)-N1-(oxetan-2-ylmethyl)benzene-1,2-diamine

To a solution of (S)-4-(5-methyl-4-((2-(trimethylsilyl)ethoxy)methyl)-4H-1,2,4-triazol-3-yl)-2-nitro-N-(oxetan-2-ylmethyl)aniline (600 mg, 1.4 mmol) in EtOH (10 mL) and H₂O (3 mL) was added Fe (401 mg, 7.2 mmol) and NH₄Cl (773 mg, 14 mmol). The mixture was stirred at 80° C. for 1 hour. The reaction mixture was filtered, poured into cold water (30 mL) and extracted with EtOAc (2×30 mL), the combined organic layers were washed with brine, dried over sodium sulfate, filtered and concentrated under reduced pressure to yield (S)-4-(5-methyl-4-((2-(trimethylsilyl)ethoxy)methyl)-4H-1,2,4-triazol-3-yl)-N1-(oxetan-2-ylmethyl) benzene-1,2-diamine (350 mg, crude) as brown oil. MS Calcd.: 389.2; MS Found: 390.3 [M+H]⁺.

### Step F: (S)-2-(chloromethyl)-5-(5-methyl-4-((2-(trimethylsilyl)ethoxy)methyl)-4H-1,2,4-triazol-3-yl)-1-(oxetan-2-ylmethyl)-1H-benzo[d]imidazole

A mixture of (S)-4-(5-methyl-4-((2-(trimethylsilyl)ethoxy)methyl)-4H-1,2,4-triazol-3-yl)-N1-(oxetan-2-ylmethyl) benzene-1,2-diamine (350 mg, crude) in THF (10 mL) was added 2-chloroacetic anhydride (630 mg, 9.5 mmol) at 0° C. The reaction mixture was stirred at 60° C. for 48 hours. The mixture was poured into cold water (20 mL) and extracted with EtOAc (2×30 mL), the combined organic layers were washed with brine, dried over sodium sulfate, filtered, and concentrated under reduced pressure. The residue was purified by column chromatography to furnish (S)-2-(chloromethyl)-5-(5-methyl-4-((2-(trimethylsilyl)ethoxy)methyl)-4H-1,2,4-triazol-3-yl)-1-(oxetan-2-ylmethyl)-1H-benzo[d]imidazole (200 mg, yield: 50%) as yellow oil. MS Calcd.: 447.2; MS Found: 448.2 [M+H]⁺.

### Step G: 2-((4-((S)-2-(4-chloro-2-fluorophenyl)-2-methylbenzo[d][1,3]dioxol-4-yl) piperidin-1-yl)methyl)-5-(5-methyl-4-((2-(trimethylsilyl)ethoxy)methyl)-4H-1,2,4-triazol-3-yl)-1-((S)-oxetan-2-ylmethyl)-1H-benzo[d]imidazole

To a solution of (S)-4-(2-(4-chloro-2-fluorophenyl)-2-methylbenzo[d][1,3]dioxol-4-yl)piperidine 4-methylbenzenesulfonate (233 mg, 0.45 mmol), K₂CO₃ (185 mg, 1.3 mmol) and TEA (136 mg, 1.3 mmol) in DMSO (5 mL) was stirred at room temperature for 10 mins. (S)-2-(chloromethyl)-5-(5-methyl-4-((2-(trimethylsilyl)ethoxy)methyl)-4H-1,2,4-triazol-3-yl)-1-(oxetan-2-ylmethyl)-1H-benzo[d]imidazole (200 mg, 0.45 mmol) was added, and the reaction mixture was stirred at 60° C. for 3 hours. The mixture was poured into cold water (20 mL) and extracted with EtOAc (2×20 mL), the combined organic layers were washed with brine, dried over sodium sulfate, filtered, and concentrated under reduced pressure. The residue was purified by column chromatography to furnish 2-((4-((S)-2-(4-chloro-2-fluorophenyl)-2-methylbenzo[d][1,3]dioxol-4-yl)piperidin-1-yl)methyl)-5-(5-methyl-4-((2-(trimethylsilyl)ethoxy)methyl)-4H-1,2,4-triazol-3-yl)-1-((S)-oxetan-2-ylmethyl)-1H-benzo[d]imidazole (200 mg, yield: 59%) as colorless oil. MS Calcd.: 758.3; MS Found: 759.3 [M+H]⁺.

### Step H: 2-({4-[(2S)-2-(4-chloro-2-fluorophenyl)-2-methyl-2H-1,3-benzodioxol-4-yl]piperidin-1-yl}methyl)-5-(5-methyl-4H-1,2,4-triazol-3-yl)-1-{[(2S)-oxetan-2-yl]methyl}-1H-1,3-benzodiazole (Compound 406a)

A mixture of 2-((4-((S)-2-(4-chloro-2-fluorophenyl)-2-methylbenzo[d][1,3]dioxol-4-yl)piperidin-1-yl)methyl)-5-(5-methyl-4-((2-(trimethylsilyl)ethoxy)methyl)-4H-1,2,4-triazol-3-yl)-1-((S)-oxetan-2-ylmethyl)-1H-benzo[d]imidazole (120 mg, 15.8 mmol) in TBAF/THF solution (10 mL) was stirred at 60° C. for 6 hours. The reaction mixture was poured into cold water (20 mL) and extracted with EtOAc (2×20 mL), the combined organic layers were washed with brine, dried over sodium sulfate, filtered, and concentrated under reduced pressure. The residue was purified by prep-HPLC to give 2-({4-[(2S)-2-(4-chloro-2-fluorophenyl)-2-methyl-2H-1,3-benzodioxol-4-yl]piperidin-1-yl}methyl)-5-(5-methyl-4H-1,2,4-triazol-3-yl)-1-{[(2S)-oxetan-2-yl]methyl}-1H-1,3-benzodiazole (Compound 406a) (36 mg, yield: 37%) as a white solid. MS Calcd.: 628.2; MS Found: 629.2 [M+H]⁺.

¹H NMR (400 MHz, MeOD): δ 8.22 (s, 1H), 7.90 (d, J=8.4 Hz, 1H), 7.63 (d, J=8.8 Hz, 1H), 7.49 (t, J=8.4 Hz, 1H), 7.18 (dd, J=8.8 Hz, 11.20 Hz, 1H), 7.11 (dd, J=6.8 Hz, 8.40 Hz, 1H), 6.72-6.60 (m, 3H), 5.21-5.15 (m, 1H), 4.72 (d, J=7.2 Hz, 1H), 4.62-4.53 (m, 2H), 4.41-4.35 (m, 1H), 4.26-4.12 (m, 2H), 3.32-3.25 (m, 1H), 2.80-2.55 (m, 4H), 2.50-2.38 (m, 1H), 2.40 (s, 3H), 2.00-1.78 (m, 8H). ¹⁹F-NMR (377 MHz): −112.28.

### Example 156: 3-{2-[(4-{3-[(4-chloro-2-fluorophenyl)methoxy]-4-fluorophenyl}-1,2,3,6-tetrahydropyridin-1-yl)methyl]-3-{[(2S)-oxetan-2-yl]methyl}-3H-imidazo[4,5-b]pyridin-6-yl}-4,5-dihydro-1,2,4-oxadiazol-5-one (Compound 293a)

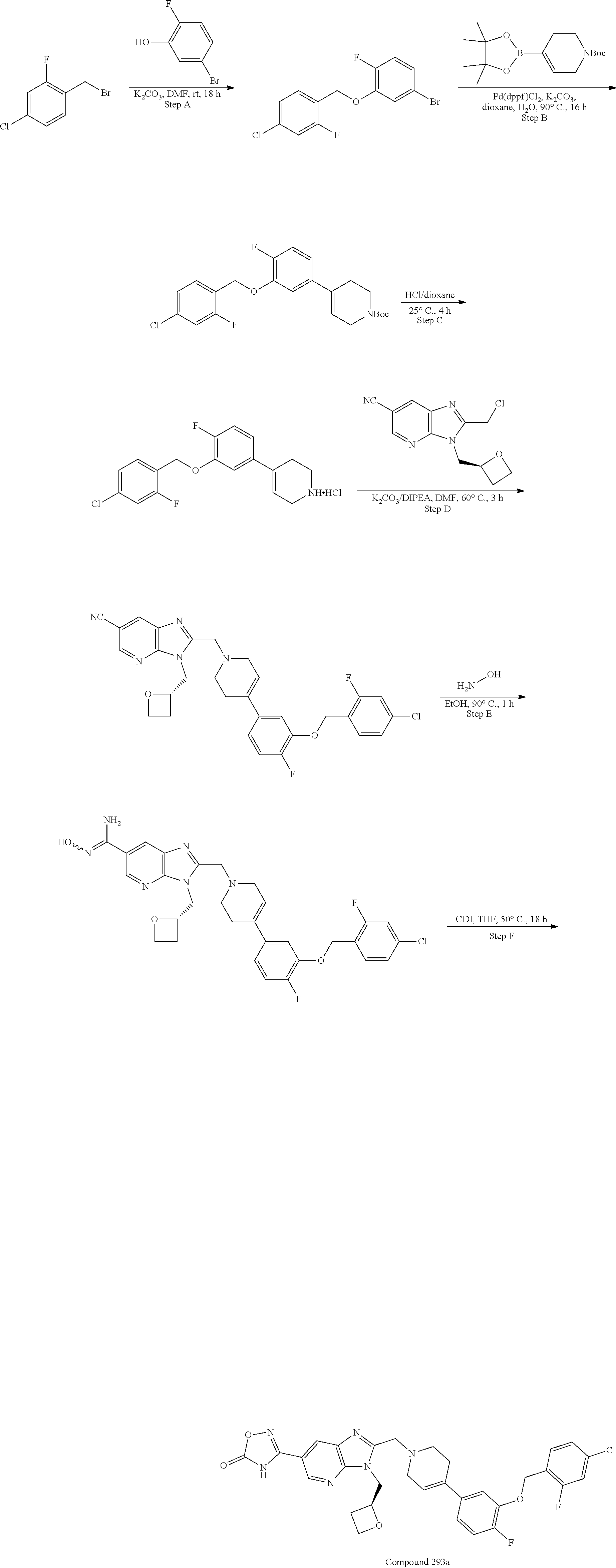

### Step A: 4-bromo-2-((4-chloro-2-fluorobenzyl)oxy)-1-fluorobenzene

A mixture of 1-(bromomethyl)-4-chloro-2-fluorobenzene (5.0 g, 22.6 mmol), 5-bromo-2-fluorophenol (5.2 g, 27.1 mmol) and K₂CO₃ (6.2 g, 45.1 mmol) in DMF (60 mL) was stirred at room temperature for 18 hours. The solid was filtrated out and washed with EA (5×20 mL). The filtrated was diluted with H₂O (600 mL) and extracted with EA (75 mL×3). The combined organic layers were washed with brine (100 mL), dried over anhydrous Na₂SO₄ and concentrated under vacuum. The residue was purified by a silica gel column to furnish 4-bromo-2-((4-chloro-2-fluorobenzyl)oxy)-1-fluorobenzene (5.4 g, crude) as crude white solid.

¹H NMR (400 MHz, CDCl₃): δ 7.46 (t, J=8.0 Hz, 1H), 7.21-7.11 (m, 3H), 7.10-7.04 (m, 1H), 7.00-6.92 (m, 1H), 5.12 (s, 2H). ¹⁹F-NMR (377 MHz): −115.82, −135.15.

### Step B: tert-butyl 4-(3-((4-chloro-2-fluorobenzyl)oxy)-4-fluorophenyl)-5,6-dihydropyridine-1(2H)-carboxylate

A mixture of 4-bromo-2-((4-chloro-2-fluorobenzyl)oxy)-1-fluorobenzene (5.3 g, 16.0 mmol, crude), tert-butyl 4-(4,4,5,5-tetramethyl-1,3,2-dioxaborolan-2-yl)-5,6-dihydropyridine-1(2H)-carboxylate (5.9 g, 19.2 mmol), Pd(dppf)Cl₂ (1.2 g, 1.6 mmol) and K₂CO₃ (4.4 g, 32.0 mmol) in H₂O/dioxane (80 mL, 1/3) was stirred at 90° C. for 16 hours. Upon cooling down, the mixture was concentrated under vacuum. The residue was purified by a silica gel column to furnish tert-butyl 4-(3-((4-chloro-2-fluorobenzyl)oxy)-4-fluorophenyl)-5,6-dihydropyridine-1(2H)-carboxylate (4.3 g, yield: 62%) as a yellow oil. MS Calcd.: 435.1; MS Found: 421.1 [M-56+H+ACN]⁺, 380.1 [M-56+H]⁺.

### Step C: 4-(3-((4-chloro-2-fluorobenzyl)oxy)-4-fluorophenyl)-1,2,3,6-tetrahydropyridine hydrochloride

A mixture of tert-butyl 4-(3-((4-chloro-2-fluorobenzyl)oxy)-4-fluorophenyl)-5,6-dihydropyridine-1(2H)-carboxylate (100 mg, 0.23 mmol) in HCl/dioxane (5.0 ml) was stirred at 25° C. for 4 hours. The reaction mixture was concentrated under vacuum to furnish 4-(3-((4-chloro-2-fluorobenzyl)oxy)-4-fluorophenyl)-1,2,3,6-tetrahydropyridine hydrochloride (110 mg, crude) as crude white solid. MS Calcd.: 335.1; MS Found: 336.0 [M+H]⁺.

### Step D: (S)-2-((4-(3-((4-chloro-2-fluorobenzyl)oxy)-4-fluorophenyl)-5,6-dihydropyridin-1(2H)-yl)methyl)-3-(oxetan-2-ylmethyl)-3H-imidazo[4,5-b]pyridine-6-carbonitrile

To a stirred mixture of 4-(3-((4-chloro-2-fluorobenzyl)oxy)-4-fluorophenyl)-1,2,3,6-tetrahydropyridine hydrochloride (110 mg, crude) and K₂CO₃ (63 mg, 0.46 mmol) in DIPEA/DMF (4.0 mL, 1/3) was added (S)-2-(chloromethyl)-3-(oxetan-2-ylmethyl)-3H-imidazo[4,5-b]pyridine-6-carbonitrile (60 mg, 0.23 mmol) at room temperature. The reaction mixture was stirred at 60° C. for 3 h. Upon cooling down, the mixture was diluted with H₂O (80 mL) and extracted with EA (15 mL×3). The combined organic layers were washed with brine (25 mL), dried over anhydrous Na₂SO₄ and concentrated under vacuum. The residue was purified by Prep-TLC to furnish (S)-2-((4-(3-((4-chloro-2-fluorobenzyl)oxy)-4-fluorophenyl)-5,6-dihydropyridin-1(2H)-yl)methyl)-3-(oxetan-2-ylmethyl)-3H-imidazo[4,5-b]pyridine-6-carbonitrile (55 mg, yield: 43%) as a white solid. MS Calcd.: 561.2; MS Found: 562.1 [M+H]⁺.

### Step E: (S)-2-((4-(3-((4-chloro-2-fluorobenzyl)oxy)-4-fluorophenyl)-5,6-dihydropyridin-1(2H)-yl)methyl)-N-hydroxy-3-(oxetan-2-ylmethyl)-3H-imidazo[4,5-b]pyridine-6-carboximidamide

A mixture of hydroxylamine (64 mg, 0.98 mmol, 50% w/w in H₂O) and (S)-2-((4-(3-((4-chloro-2-fluorobenzyl)oxy)-4-fluorophenyl)-5,6-dihydropyridin-1(2H)-yl)methyl)-3-(oxetan-2-ylmethyl)-3H-imidazo[4,5-b]pyridine-6-carbonitrile (55 mg, 0.098 mmol) in EtOH (3.0 ml) was stirred at 90° C. for 1 hour in a sealed tube. Upon cooling down, the reaction mixture was directly concentrated under vacuum to furnish (S)-2-((4-(3-((4-chloro-2-fluorobenzyl)oxy)-4-fluorophenyl)-5,6-dihydropyridin-1(2H)-yl)methyl)-N-hydroxy-3-(oxetan-2-ylmethyl)-3H-imidazo[4,5-b]pyridine-6-carboximidamide (60 mg, crude) as crude yellow solid. MS Calcd.: 594.2; MS Found: 595.2 [M+H]⁺.

### Step F: 3-{2-[(4-{3-[(4-chloro-2-fluorophenyl)methoxy]-4-fluorophenyl}-1,2,3,6-tetrahydropyridin-1-yl)methyl]-3-{[(2S)-oxetan-2-yl]methyl}-3H-imidazo[4,5-b]pyridin-6-yl}-4,5-dihydro-1,2,4-oxadiazol-5-one (Compound 293a)

A mixture of CDI (22 mg, 0.13 mmol) and (S)-2-((4-(3-((4-chloro-2-fluorobenzyl)oxy)-4-fluorophenyl)-5,6-dihydropyridin-1(2H)-yl)methyl)-N-hydroxy-3-(oxetan-2-ylmethyl)-3H-imidazo[4,5-b]pyridine-6-carboximidamide (20 mg, crude) in THE (4.0 ml) was stirred at 50° C. for 18 hours. Upon cooling down, the mixture was directly purified by Prep-HPLC to furnish 3-{2-[(4-{3-[(4-chloro-2-fluorophenyl)methoxy]-4-fluorophenyl}-1,2,3,6-tetrahydropyridin-1-yl)methyl]-3-{[(2S)-oxetan-2-yl]methyl}-3H-imidazo[4,5-b]pyridin-6-yl}-4,5-dihydro-1,2,4-oxadiazol-5-one (Compound 293a) (5.2 mg, 24%) as a white solid.

MS Calcd.: 620.2; MS Found: 621.2 [M+H]⁺.

¹H NMR (400 MHz, MeOD): δ 8.84 (d, J=2.0 Hz, 1H), 8.42 (d, J=2.0 Hz, 1H), 7.52 (t, J=8.0 Hz, 2H), 7.26-7.17 (m, 3H), 7.09-7.01 (m, 2H), 6.10-6.05 (m, 1H), 5.31-5.24 (m, 1H), 5.19 (s, 2H), 4.98-4.90 (m, 1H), 4.79 (dd, J=14.4, 3.2 Hz, 1H), 4.62 (dd, J=13.6, 8.4 Hz, 1H), 4.44-4.39 (m, 1H), 4.26-4.17 (m, 2H), 3.36-3.30 (m, 2H), 2.93 (t, J=5.6 Hz, 2H), 2.80-2.70 (m, 1H), 2.61-2.49 (m, 3H). ¹⁹F-NMR (377 MHz): −117.53, −137.69.

### Example 157: 4-{3-[(4-chloro-2-fluorophenyl)methoxy]-4-fluorophenyl}-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1,2,3,6-tetrahydropyridine (Compound 294a)

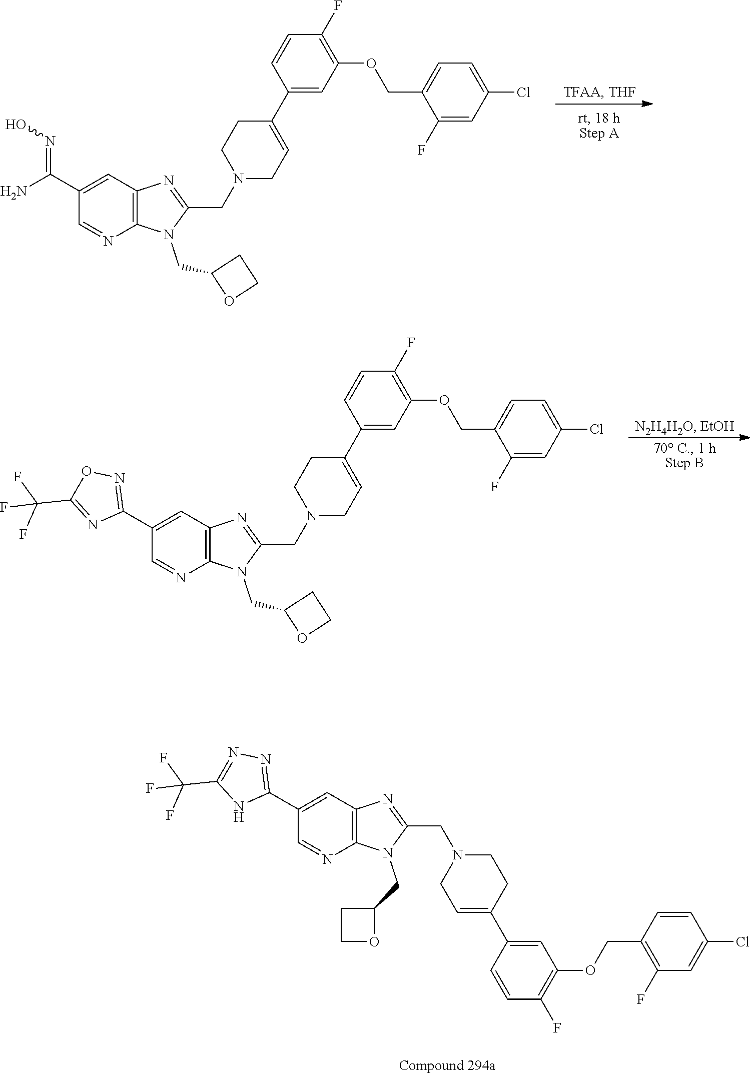

### Step A: (S)-3-(2-((4-(3-((4-chloro-2-fluorobenzyl)oxy)-4-fluorophenyl)-5,6-dihydropyridin-1(2H)-yl)methyl)-3-(oxetan-2-ylmethyl)-3H-imidazo[4,5-b]pyridin-6-yl)-5-(trifluoromethyl)-1,2,4-oxadiazole

To a stirred solution of (S)-2-((4-(3-((4-chloro-2-fluorobenzyl)oxy)-4-fluorophenyl)-5,6-dihydropyridin-1(2H)-yl)methyl)-N′-hydroxy-3-(oxetan-2-ylmethyl)-3H-imidazo[4,5-b]pyridine-6-carboximidamide (260 mg, 0.44 mmol) in THE (5.0 mL) was added a solution of TFAA (460 mg, 2.2 mmol) in THE (1.0 mL) dropwisely at 0° C. The final reaction mixture was stirred at room temperature for 18 hours. The mixture was diluted with saturated NaHCO₃ (80 mL) and extracted with EA (15 mL×3). The combined organic layers were washed with brine (10 mL), dried over anhydrous Na₂SO₄, and concentrated under vacuum. The residue was purified by Prep-TLC to furnish (S)-3-(2-((4-(3-((4-chloro-2-fluorobenzyl)oxy)-4-fluorophenyl)-5,6-dihydropyridin-1(2H)-yl)methyl)-3-(oxetan-2-ylmethyl)-3H-imidazo[4,5-b]pyridin-6-yl)-5-(trifluoromethyl)-1,2,4-oxadiazole (180 mg, yield: 61%) as a white solid. MS Calcd.: 672.2; MS Found: 673.1 [M+H]⁺.

### Step B: 4-{3-[(4-chloro-2-fluorophenyl)methoxy]-4-fluorophenyl}-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1,2,3,6-tetrahydropyridine (Compound 294a)

A mixture of N₂H₄H₂O (1.0 mL) and (S)-3-(2-((4-(3-((4-chloro-2-fluorobenzyl)oxy)-4-fluorophenyl)-5,6-dihydropyridin-1(2H)-yl)methyl)-3-(oxetan-2-ylmethyl)-3H-imidazo[4,5-b]pyridin-6-yl)-5-(trifluoromethyl)-1,2,4-oxadiazole (180 mg, 0.27 mmol) in EtOH (3.0 mL) was stirred at 70° C. for 1 hour. Upon cooling down, the mixture was directly purified by Prep-HPLC (0.1% FA) to furnish 4-{3-[(4-chloro-2-fluorophenyl)methoxy]-4-fluorophenyl}-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1,2,3,6-tetrahydropyridine (Compound 294a) (52 mg, 29%) as a white solid.

MS Calcd.: 671.2; MS Found: 672.5 [M+H]⁺.

¹H NMR (400 MHz, MeOD): δ 9.05 (d, J=2.0 Hz, 1H), 8.62 (d, J=2.0 Hz, 1H), 7.52 (t, J=8.0 Hz, 2H), 7.25-7.17 (m, 3H), 7.05-7.00 (m, 2H), 6.10-6.05 (m, 1H), 5.33-5.27 (m, 1H), 5.18 (s, 2H), 4.99-4.94 (m, 1H), 4.80 (dd, J=14.8, 3.2 Hz, 1H), 4.62 (dd, J=13.2, 7.2 Hz, 1H), 4.47-4.41 (m, 1H), 4.21-4.12 (m, 2H), 3.31-3.26 (m, 2H), 2.87 (t, J=5.6 Hz, 2H), 2.79-2.73 (m, 1H), 2.57-2.50 (m, 3H). ¹⁹F-NMR (377 MHz): −66.55, −117.54, −137.82.

### Example 158: 3-fluoro-4-[(2-fluoro-5-{1-[(3-{[(2S)-oxetan-2-yl]methyl}-6-(5-oxo-4,5-dihydro-1,2,4-oxadiazol-3-yl)-3H-imidazo[4,5-b]pyridin-2-yl)methyl]-1,2,3,6-tetrahydropyridin-4-yl}phenoxy)methyl]benzonitrile (Compound 295a)

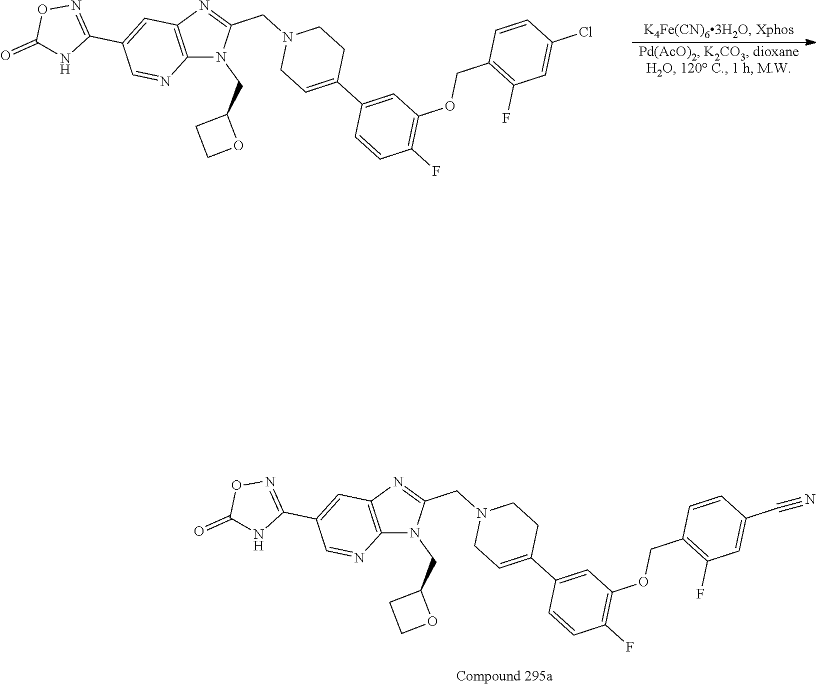 [SMILES: O=c1[nH]c(-c2cnc3c(c2)nc(CN2CC=C(c4ccc(F)c(OCc5ccc(Cl)cc5F)c4)CC2)n3C[C@@H]2CCO2)no1 ; "K4Fe(CN)6·3H2O, Xphos" ; "Pd(AcO)2, K2CO3, dioxaneH2O, 120° C., 1 h, M.W." ; N#Cc1ccc(COc2cc(C3=CCN(Cc4nc5cc(-c6noc(=O)[nH]6)cnc5n4C[C@@H]4CCO4)CC3)ccc2F)c(F)c1]

A mixture of Pd(OAc)₂ (1.9 mg, 0.009 mmol), K₄Fe(CN)₆·3H₂O (37 mg, 0.09 mmol), K₂CO₃ (37 mg, 0.27 mmol), X-Phos (8.5 mg, 0.018 mmol), (S)-2-((4-(3-((4-chloro-2-fluorobenzyl)oxy)-4-fluorophenyl)-5,6-dihydropyridin-1(2H)-yl)methyl)-3-(oxetan-2-ylmethyl)-6-(5-(trifluoromethyl)-4H-1,2,4-triazol-3-yl)-3H-imidazo[4,5-b]pyridine formate (55 mg, 0.09 mmol) in H₂O/dioxane (1.8 mL, 1/5) was stirred at 120° C. for 1 hour under Ar atmosphere with irradiation. Upon cooling down, the mixture was directly purified by Prep-HPLC to furnish 3-fluoro-4-[(2-fluoro-5-{1-[(3-{[(2S)-oxetan-2-yl]methyl}-6-(5-oxo-4,5-dihydro-1,2,4-oxadiazol-3-yl)-3H-imidazo[4,5-b]pyridin-2-yl)methyl]-1,2,3,6-tetrahydropyridin-4-yl}phenoxy)methyl]benzonitrile (Compound 295a) (3.4 mg, 6.2%) as a white solid.

MS Calcd.: 611.2; MS Found: 612.2 [M+H]⁺.

¹H NMR (400 MHz, MeOD): δ 8.83 (d, J=2.0 Hz, 1H), 8.42 (d, J=1.6 Hz, 1H), 7.76 (t, J=8.6 Hz, 1H), 7.64-7.58 (m, 2H), 7.21 (dd, J=8.0, 1.6 Hz, 1H), 7.10-7.00 (m, 2H), 6.11-6.05 (m, 1H), 5.40-5.25 (m, 3H), 4.94 (dd, J=14.8, 6.4 Hz, 1H), 4.78 (dd, J=14.4, 3.2 Hz, 1H), 4.64-4.58 (m, 1H), 4.44-4.38 (m, 1H), 4.27-4.17 (m, 2H), 3.37-3.30 (m, 2H), 2.83 (t, J=6.0 Hz, 2H), 2.80-2.70 (m, 1H), 2.62-2.49 (m, 3H). ¹⁹F-NMR (377 MHz): −117.23, −137.78.

### Example 159: 3-fluoro-4-[(2-fluoro-5-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1,2,3,6-tetrahydropyridin-4-yl}phenoxy)methyl]benzonitrile (Compound 296a)

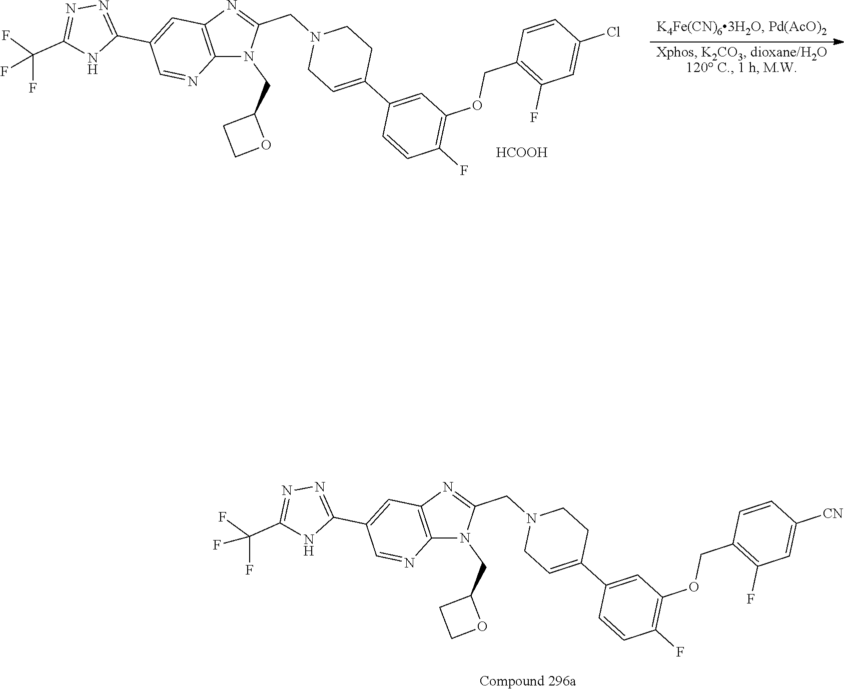 [SMILES: Fc1cc(Cl)ccc1COc1cc(C2=CCN(Cc3nc4cc(-c5nnc(C(F)(F)F)[nH]5)cnc4n3C[C@@H]3CCO3)CC2)ccc1F ; "HCOOH" ; "K4Fe(CN)6·3H2O, Pd(AcO)2" ; "Xphos, K2CO3, dioxane/H2O120° C., 1 h, M.W." ; N#Cc1ccc(COc2cc(C3=CCN(Cc4nc5cc(-c6nnc(C(F)(F)F)[nH]6)cnc5n4C[C@@H]4CCO4)CC3)ccc2F)c(F)c1]

A mixture of K₄Fe(CN)₆·3H₂O (25 mg, 0.06 mmol), Pd(OAc)₂ (1.3 mg, 0.006 mmol), X-Phos (5.7 mg, 0.012 mmol), K₂CO₃ (28 mg, 0.18 mmol), (S)-2-((4-(3-((4-chloro-2-fluorobenzyl)oxy)-4-fluorophenyl)-5,6-dihydropyridin-1(2H)-yl)methyl)-3-(oxetan-2-ylmethyl)-6-(5-(trifluoromethyl)-4H-1,2,4-triazol-3-yl)-3H-imidazo[4,5-b]pyridine formate (40 mg, 0.06 mmol) in H₂O/dioxane (1.2 mL, 1/5) was stirred at 120° C. for 1 hour under Ar atmosphere with irradiation. Upon cooling down, the mixture was directly purified by Prep-HPLC to furnish 3-fluoro-4-[(2-fluoro-5-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1,2,3,6-tetrahydropyridin-4-yl}phenoxy)methyl]benzonitrile (Compound 296a) (1.0 mg, 2.5%) as a white solid.

MS Calcd.: 662.2; MS Found: 663.2 [M+H]⁺.

¹H NMR (400 MHz, MeOD): δ 9.08 (d, J=1.6 Hz, 1H), 8.65 (d, J=2.0 Hz, 1H), 7.76 (t, J=7.6 Hz, 1H), 7.63-7.57 (m, 2H), 7.24-7.20 (m, 1H), 7.09-7.02 (m, 2H), 6.10-6.07 (m, 1H), 5.31-5.28 (m, 3H), 4.64-4.55 (m, 3H), 4.47-4.39 (m, 1H), 4.14-4.11 (m, 2H), 3.30-3.24 (m, 2H), 2.85 (t, J=5.6 Hz, 2H), 2.79-2.71 (m, 1H), 2.59-2.51 (m, 3H). ¹⁹F-NMR (377 MHz): −64.53, −117.19, −138.09.

### Example 160: 3-{2-[(4-{3-[(4-chloro-2-fluorophenyl)methoxy]phenyl}-1,2,3,6-tetrahydropyridin-1-yl)methyl]-3-{[(2S)-oxetan-2-yl]methyl}-3H-imidazo[4,5-b]pyridin-6-yl}-4,5-dihydro-1,2,4-oxadiazol-5-one (Compound 320a)

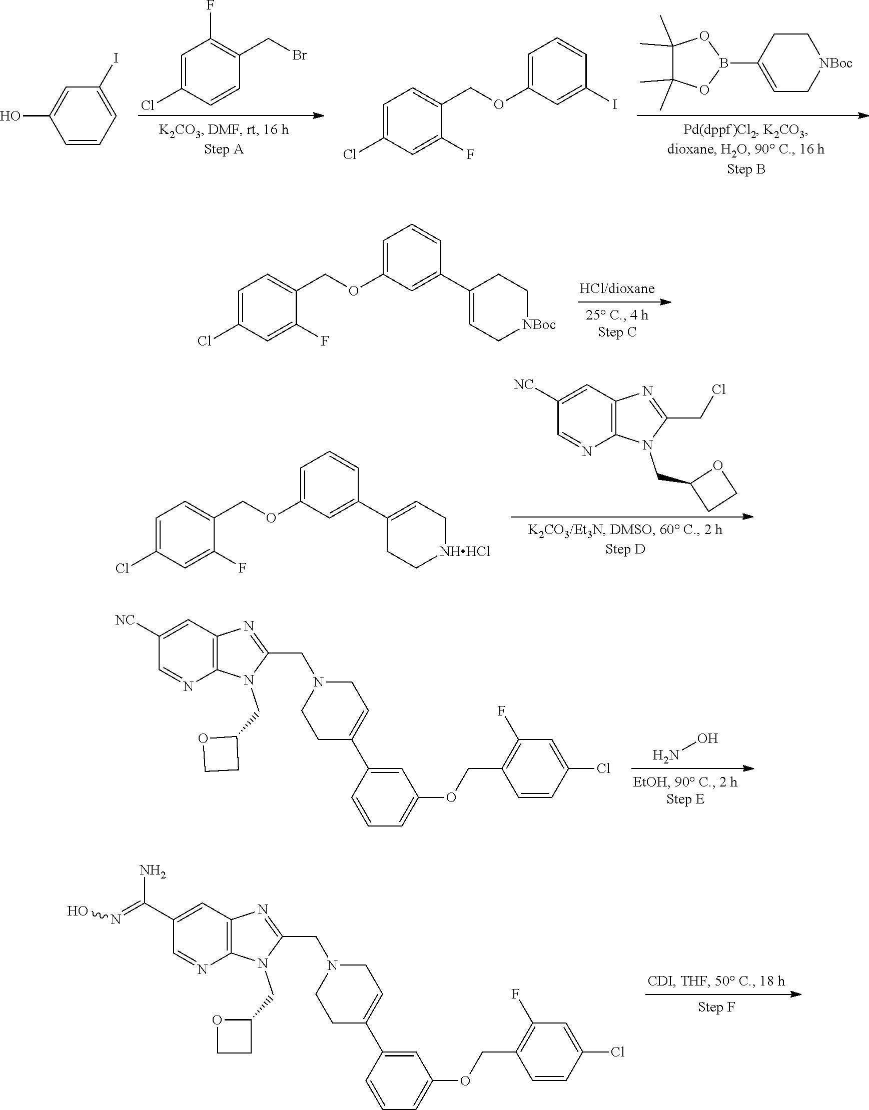

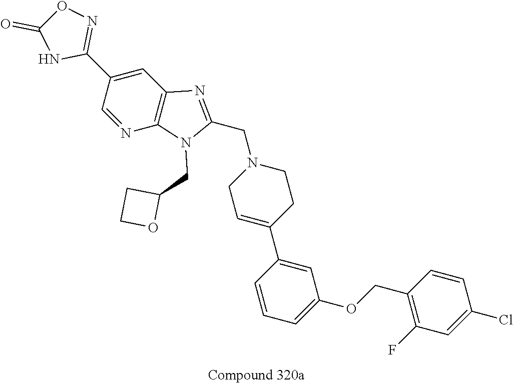 [SMILES: O=c1[nH]c(-c2cnc3c(c2)nc(CN2CC=C(c4cccc(OCc5ccc(Cl)cc5F)c4)CC2)n3C[C@@H]2CCO2)no1]

### Step A: 4-chloro-2-fluoro-1-((3-iodophenoxy)methyl)benzene

A mixture of 3-iodophenol (4.5 g, 20.4 mmol), 1-(bromomethyl)-4-chloro-2-fluorobenzene (4.5 g, 20.4 mmol) and K₂CO₃ (8.5 g, 61.4 mmol) in DMF (80 mL) was stirred at room temperature for 16 hours. The solid was filtrated out and washed with EA (5×20 mL). The filtrated was diluted with H₂O (600 mL) and extracted with EA (3×75 mL). The combined organic layers were washed with brine (100 mL), dried over anhydrous Na₂SO₄ and concentrated under vacuum. The residue was purified by a silica gel column to furnish 4-chloro-2-fluoro-1-((3-iodophen oxy)methyl)benzene (4.5 g, yield: 62%) as a yellow oil.

### Step B: tert-butyl 4-(3-((4-chloro-2-fluorobenzyl)oxy)phenyl)-5,6-dihydropyridine-1(2H)-carboxylate

A mixture of 4-chloro-2-fluoro-1-((3-iodophenoxy)methyl)benzene (4.0 g, 11.0 mmol), tert-butyl 4-(4,4,5,5-tetramethyl-1,3,2-dioxaborolan-2-yl)-5,6-dihydropyridine-1(2H)-carboxylate (3.4 g, 11.0 mm ol), Pd(dppf)Cl₂ (809 mg, 1.1 mmol) and K₂CO₃ (4.6 g, 33.1 mmol) in H₂O/dioxane (80 mL, 1/3) was stirred at 90° C. for 16 hours. Upon cooling down, the mixture was concentrated under vacuum. The residue was purified by a silica gel column to furnish 4-(3-((4-chloro-2-fluorobenzyl)oxy)phenyl)-5,6-dihydropyridine-1(2H)-carboxylate (2.6 g, yield: 56%) as a yellow oil. MS Calcd.: 417.2; MS Found: 362.1 [M-56+H]⁺, 318.1 [M-100+H]⁺.

### Step C: 4-(3-((4-chloro-2-fluorobenzyl)oxy)phenyl)-1,2,3,6-tetrahydropyridine hydrochloride

A mixture of 4-(3-((4-chloro-2-fluorobenzyl)oxy)phenyl)-5,6-dihydropyridine-1(2H)-carboxylate (1.6 g, 3.8 mmol) in HCl/dioxane (20.0 mL, 4M) was stirred at 25° C. for 4 hours. The reaction mixture was concentrated under vacuum to furnish 4-(3-((4-chloro-2-fluorobenzyl)oxy)phenyl)-1,2,3,6-tetrahydropyridine hydrochloride (1.1 g, crude) as crude white solid. MS Calcd.: 317.1; MS Found: 318.1 [M+H]⁺.

### Step D: (S)-2-((4-(3-((4-chloro-2-fluorobenzyl)oxy)phenyl)-5,6-dihydropyridin-1(2H)-yl) methyl)-3-(oxetan-2-ylmethyl)-3H-imidazo[4,5-b]pyridine-6-carbonitrile

To a stirred mixture of 4-(3-((4-chloro-2-fluorobenzyl)oxy)phenyl)-1,2,3,6-tetrahydropyridine hydrochloride (200 mg, 0.76 mmol, crude) and K₂CO₃ (316 mg, 2.3 mmol), TEA (231 mg, 2.3 mmol) in DMSO (5.0 mL) was added (S)-2-(chloromethyl)-3-(oxetan-2-ylmethyl)-3H-imidazo[4,5-b]pyridine-6-carbonitrile (269 mg, 0.76 mmol) at room temperature. The reaction mixture was stirred at 60° C. for 2 h. Upon cooling down, the mixture was diluted with H₂O (80 mL) and extracted with EA (15 mL×3). The combined organic layers were washed with brine (25 mL), dried over anhydrous Na₂SO₄ and concentrated under vacuum. The residue was purified by Prep-TLC to give (S)-2-((4-(3-((4-chloro-2-fluorobenzyl)oxy) phenyl)-5,6-dihydropyridin-1(2H)-yl)methyl)-3-(oxetan-2-ylmethyl)-3H-imidazo[4,5-b]pyridine-6-carbonitrile (190 mg, yield: 46%) as a white solid. MS Calcd.: 543.2; MS Found: 544.3 [M+H]⁺.

### Step E: (S)-2-((4-(3-((4-chloro-2-fluorobenzyl)oxy)phenyl)-5,6-dihydropyridin-1(2H)-yl)methyl)-N′-hydroxy-3-(oxetan-2-ylmethyl)-3H-imidazo[4,5-b]pyridine-6-carboximidamide

A mixture of hydroxylamine (230 mg, 3.5 mmol, 50% w/w in H₂O) and (S)-2-((4-(3-((4-chloro-2-fluorobenzyl)oxy)phenyl)-5,6-dihydropyridin-1(2H)-yl)methyl)-3-(oxetan-2-ylmethyl)-3H-imidazo[4,5-b]pyridine-6-carbonitrile (190 mg, 0.35 mmol) in EtOH (5.0 ml) was stirred at 90° C. for 2 hour in a sealed tube. Upon cooling down, the reaction mixture was directly concentrated under vacuum to give (S)-2-((4-(3-((4-chloro-2-fluorobenzyl)oxy)phenyl)-5,6-dihydropyridin-1(2H)-yl)methyl)-N′-hydroxy-3-(oxetan-2-ylmethyl)-3H-imidazo[4,5-b]pyridine-6-carboximidamide (210 mg, crude) as crude yellow solid. MS Calcd.: 576.2; MS Found: 577.1 [M+H]⁺.

### Step F: 3-{2-[(4-{3-[(4-chloro-2-fluorophenyl)methoxy]phenyl}-1,2,3,6-tetrahydropyridin-1-yl)methyl]-3-{[(2S)-oxetan-2-yl]methyl}-3H-imidazo[4,5-b]pyridin-6-yl}-4,5-dihydro-1,2,4-oxadiazol-5-one (Compound 320a)

A mixture of CDI (84 mg, 0.52 mmol) and (S)-2-((4-(3-((4-chloro-2-fluorobenzyl)oxy)phenyl)-5,6-dihydropyridin-1(2H)-yl)methyl)-N′-hydroxy-3-(oxetan-2-ylmethyl)-3H-imidazo[4,5-b]pyridine-6-carboximidamide (60 mg, crude) in THE (5.0 ml) was stirred at 50° C. for 18 hours. Upon cooling down, the mixture was directly purified by Prep-HPLC to furnish 3-{2-[(4-{3-[(4-chloro-2-fluorophenyl)methoxy]phenyl}-1,2,3,6-tetrahydropyridin-1-yl)methyl]-3-{[(2S)-oxetan-2-yl]methyl}-3H-imidazo[4,5-b]pyridin-6-yl}-4,5-dihydro-1,2,4-oxadiazol-5-one (Compound 320a) (18 mg, 29%) as a white solid.

MS Calcd.: 602.2; MS Found: 603.2 [M+H]⁺.

¹H NMR (400 MHz, DMSO-d₆): δ 8.78 (d, J=1.6 Hz, 1H), 8.39 (d, J=1.2 Hz, 1H), 7.58 (t, J=8.0 Hz, 1H), 7.51-7.47 (m, 1H), 7.35-7.32 (m, 1H), 7.26 (d, J=7.6 Hz, 1H), 7.08-7.01 (m, 2H), 6.94-6.88 (m, 1H), 6.19 (br.s, 1H), 5.20-5.10 (m, 3H), 4.87-4.78 (m, 1H), 4.72-4.67 (m, 1H), 4.50-4.42 (m, 1H), 4.39-4.32 (m, 1H), 4.13-4.00 (m, 2H), 3.26-3.18 (m, 3H), 2.78 (t, J=5.2 Hz, 2H), 2.71-2.60 (m, 1H), 2.49-2.40 (m, 2H). ¹⁹F-NMR (377 MHz): −115.14.

### Example 161: 3-fluoro-4-[(3-{1-[(3-{[(2S)-oxetan-2-yl]methyl}-6-(5-oxo-4,5-dihydro-1,2,4-oxadiazol-3-yl)-3H-imidazo[4,5-b]pyridin-2-yl)methyl]-1,2,3,6-tetrahydropyridin-4-yl}phenoxy)methyl]benzonitrile (Compound 318a)

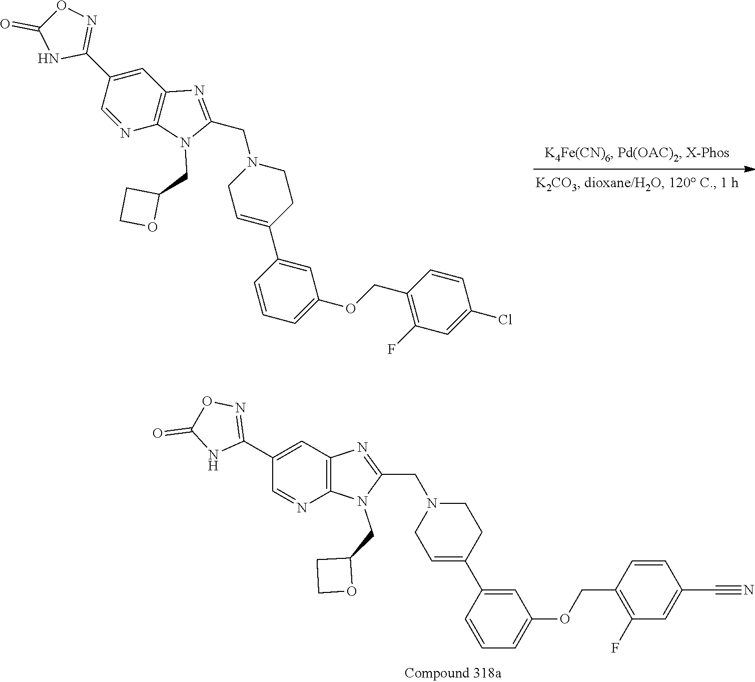 [SMILES: O=c1[nH]c(-c2cnc3c(c2)nc(CN2CC=C(c4cccc(OCc5ccc(Cl)cc5F)c4)CC2)n3C[C@@H]2CCO2)no1 ; N#Cc1ccc(COc2cccc(C3=CCN(Cc4nc5cc(-c6noc(=O)[nH]6)cnc5n4C[C@@H]4CCO4)CC3)c2)c(F)c1]

A mixture of Pd(OAc)₂ (0.6 mg, 0.003 mmol), K₄Fe(CN)₆·3H₂O (11 mg, 0.03 mmol), K₂CO₃ (11 mg, 0.08 mmol), X-Phos (2.4 mg, 0.005 mmol), (S)-3-(2-((4-(3-((4-chloro-2-fluorobenzyl)oxy)phenyl)-5,6-dihydropyridin-1(2H)-yl)methyl)-3-(oxetan-2-ylmethyl)-3H-imidazo[4,5-b]pyridin-6-yl)-1,2,4-oxadiazol-5(4H)-one (16 mg, 0.04 mmol) in H₂O/dioxane (1.2 ml, 1/5) was stirred at 120° C. for 1 hour under Ar atmosphere with irradiation. Upon cooling down, the mixture was directly purified by Prep-HPLC to furnish 3-fluoro-4-[(3-{1-[(3-{[(2S)-oxetan-2-yl]methyl}-6-(5-oxo-4,5-dihydro-1,2,4-oxadiazol-3-yl)-3H-imidazo[4,5-b]pyridin-2-yl)methyl]-1,2,3,6-tetrahydropyridin-4-yl}phenoxy)methyl]benzonitrile (Compound 318a) (1.7 mg, 11%) as a white solid.

MS Calcd.: 593.2; MS Found: 594.2 [M+H]⁺.

¹H NMR (400 MHz, MeOD): δ 8.75 (m, 1H), 8.36-8.32 (m, 1H), 7.65 (t, J=7.6 Hz, 1H), 7.53-7.48 (m, 2H), 7.20-7.12 (t, J=8.2 Hz, 1H), 7.00-6.95 (m, 2H), 6.82-6.78 (m, 1H), 6.03 (br.s, 1H), 5.25-5.15 (m, 1H), 5.13 (s, 2H), 4.88-4.81 (m, 1H), 4.75-4.65 (m, 1H), 4.55-4.48 (m, 1H), 4.35-4.29 (m, 1H), 4.20-4.10 (m, 2H), 3.28 (br.s, 2H), 2.90-2.83 (m, 2H), 2.71-2.60 (m, 1H), 2.58-2.50 (m, 2H), 2.49-2.38 (m, 1H). ¹⁹F-NMR (377 MHz): −114.45.

### Example 162: 4-{3-[(4-chloro-2-fluorophenyl)methoxy]phenyl}-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1,2,3,6-tetrahydropyridine (Compound 319a)

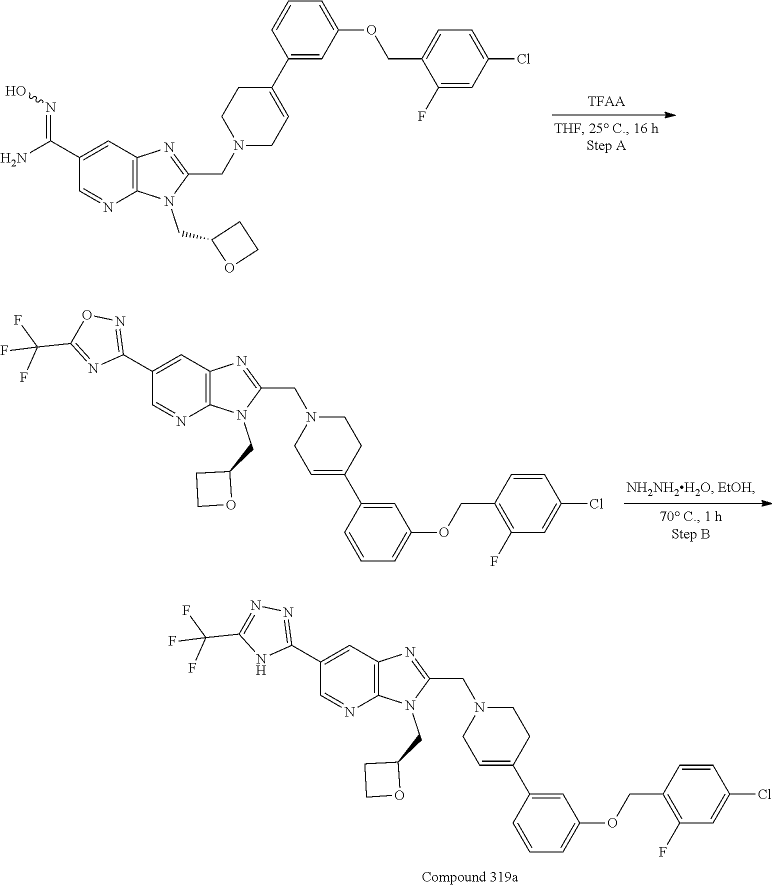

### Step A: (S)-3-(2-((4-(3-((4-chloro-2-fluorobenzyl)oxy)phenyl)-5,6-dihydropyridin-1(2H)-yl)methyl)-3-(oxetan-2-ylmethyl)-3H-imidazo[4,5-b]pyridin-6-yl)-5-(trifluoromethyl)-1,2,4-oxadiazole

To a stirred solution of (S)-2-((4-(3-((4-chloro-2-fluorobenzyl)oxy)phenyl)-5,6-dihydropyridin-1(2H)-yl)methyl)-N′-hydroxy-3-(oxetan-2-ylmethyl)-3H-imidazo[4,5-b]pyridine-6-carboximidamide (60 mg) in THF (5.0 mL) was added a solution of TFAA (109 mg, 0.52 mmol) in THF (2.0 mL) dropwisely at 0° C. The final reaction mixture was stirred at room temperature for 16 hours. The reaction mixture was quenched by saturated NaHCO₃ (75 mL) and extracted with EA (30 mL×3). The combined organic layers were dried over anhydrous Na₂SO₄ and concentrated under vacuum to give (S)-3-(2-((4-(3-((4-chloro-2-fluorobenzyl)oxy)phenyl)-5,6-dihydropyridin-1(2H)-yl)methyl)-3-(oxetan-2-ylmethyl)-3H-imidazo[4,5-b]pyridin-6-yl)-5-(trifluoromethyl)-1,2,4-oxadiazole (65 mg, crude) as crude white solid. MS Calcd.: 654.2; MS Found: 655.2 [M+H]⁺.

### Step B: 4-{3-[(4-chloro-2-fluorophenyl)methoxy]phenyl}-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1,2,3,6-tetrahydropyridine (Compound 319a)

A mixture of N₂H₄H₂O (25 mg, 0.50 mmol) and (S)-3-(2-((4-(3-((4-chloro-2-fluorobenzyl)oxy)phenyl)-5,6-dihydropyridin-1(2H)-yl)methyl)-3-(oxetan-2-ylmethyl)-3H-imidazo[4,5-b]pyridin-6-yl)-5-(trifluoromethyl)-1,2,4-oxadiazole (65 mg, crude) in EtOH (2 mL) was stirred at 70° C. for 1 hour. Upon cooling down, the reaction mixture was directly purified by Prep-HPLC to furnish 4-{3-[(4-chloro-2-fluorophenyl)methoxy]phenyl}-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1,2,3,6-tetrahydropyridine (Compound 319a) (5.9 mg, 9%) as a white solid.

MS Calcd.: 653.2; MS Found: 654.2 [M+H]⁺.

¹H NMR (400 MHz, MeOD): δ 9.05 (d, J=1.6 Hz, 1H), 8.62 (d, J=2.0 Hz, 1H), 7.51 (t, J=8.2 Hz, 1H), 7.28-7.20 (m, 3H), 7.07-7.00 (m, 2H), 6.90-6.83 (m, 1H), 6.12 (br.s, 1H), 5.35-5.25 (m, 1H), 5.12 (s, 2H), 5.01-4.93 (m, 1H), 4.68-4.59 (m, 2H), 4.46-4.40 (m, 1H), 4.21-4.10 (m, 2H), 2.91-2.85 (t, J=5.6 Hz, 2H), 2.83-2.73 (m, 1H), 2.62-2.50 (m, 3H). (2H overlapped with MeOD peaks at 3.20-3.40). ¹⁹F-NMR (377 MHz): −66.57, −117.79, −117.80, −117.81.

### Example 163: 3-[2-({6-[(4-chloro-2-fluorophenyl)methoxy]-1′,2′,3′,6′-tetrahydro-[2,4′-bipyridin]-1′-yl}methyl)-3-{[(2S)-oxetan-2-yl]methyl}-3H-imidazo[4,5-b]pyridin-6-yl]-4,5-dihydro-1,2,4-oxadiazol-5-one (Compound 324a)

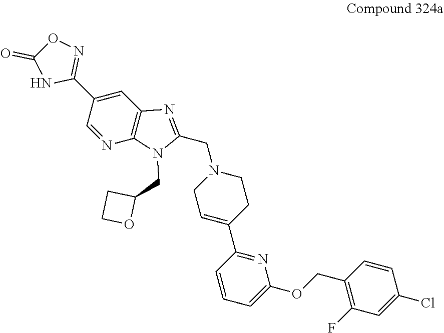 [SMILES: O=c1[nH]c(-c2cnc3c(c2)nc(CN2CC=C(c4cccc(OCc5ccc(Cl)cc5F)n4)CC2)n3C[C@@H]2CCO2)no1]

Starting from 6-((4-chloro-2-fluorobenzyl)oxy)-1′,2′,3′,6′-tetrahydro-2,4′-bipyridinehydrochloride and (S)-2-(chloromethyl)-3-(oxetan-2-ylmethyl)-3H-imidazo[4,5-b]pyridine-6-carbonitrile, 3-[2-({6-[(4-chloro-2-fluorophenyl)methoxy]-1′,2′,3′,6′-tetrahydro-[2,4′-bipyridin]-1′-yl}methyl)-3-{[(2S)-oxetan-2-yl]methyl}-3H-imidazo[4,5-b]pyridin-6-yl]-4,5-dihydro-1,2,4-oxadiazol-5-one (Compound 324a) (9.7 mg) was obtained with the similar procedure of Compound 397a as white solid.

MS Calcd: 603.2; MS Found: 604.2 [M+H]⁺.

¹H NMR (400 MHz, DMSO-d₆) δ 8.77 (d, J=1.6 Hz, 1H), 8.32 (s, 1H), 7.69 (t, J=8.0 Hz, 1H), 7.56 (t, J=8.4 Hz, 1H), 7.46 (dd, J=1.6 Hz, J=10.0 Hz, 1H), 7.30 (dd, J=1.6 Hz, J=8.0 Hz, 1H), 7.09 (d, J=7.2 Hz, 1H), 6.79-6.70 (m, 2H), 5.40 (s, 2H), 5.19-5.10 (m, 1H), 4.83-4.77 (m, 1H), 4.71-4.65 (m, 1H), 4.50-4.43 (m, 1 H), 4.40-4.31 (m, 1H), 4.11-4.00 (m, 2H), 3.28 (br.s, 2H), 2.77 (t, J=5.2 Hz, 2H), 2.70-2.60 (m, 1H), 2.58-2.50 (m, 2H), 2.50-2.40 (m, 1H). ¹⁹F-NMR (377 MHz): −115.25.

### Example 164: 3-fluoro-4-[({1′-[(3-{[(2S)-oxetan-2-yl]methyl}-6-(5-oxo-4,5-dihydro-1,2,4-oxadiazol-3-yl)-3H-imidazo[4,5-b]pyridin-2-yl)methyl]-1′,2′,3′,6′-tetrahydro-[2,4′-bipyridin]-6-yl}oxy)methyl]benzonitrile (Compound 322a)

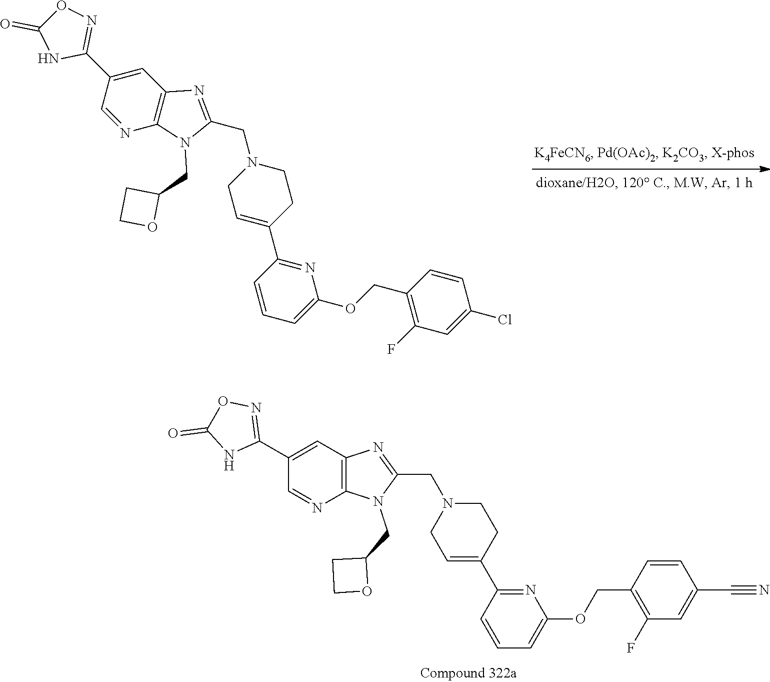 [SMILES: O=c1[nH]c(-c2cnc3c(c2)nc(CN2CC=C(c4cccc(OCc5ccc(Cl)cc5F)n4)CC2)n3C[C@@H]2CCO2)no1 ; N#Cc1ccc(COc2cccc(C3=CCN(Cc4nc5cc(-c6noc(=O)[nH]6)cnc5n4C[C@@H]4CCO4)CC3)n2)c(F)c1]

A mixture of 3-[2-({6-[(4-chloro-2-fluorophenyl)methoxy]-1′,2′,3′,6′-tetrahydro-[2,4′-bipyridin]-1′-yl}methyl)-3-{[(2S)-oxetan-2-yl]methyl}-3H-imidazo[4,5-b]pyridin-6-yl]-4,5-dihydro-1,2,4-oxadiazol-5-one (40 mg, 0.066 mmol), K₄Fe(CN)₆ (28 mg, 0.066 mmol), Pd(OAc)₂ (1.5 mg, 0.0066 mmol), Xphos (6.3 mg, 0.013 mmol) and K₂CO₃ (27 mg, 0.20 mmol) in 1,4-dioxane/H₂O (1 mL/0.2 mL) was stirred at 120° C. with M.W under Ar for 1 hours. The mixture was filtered and the filtrate was purified by prep-HPLC to obtain 3-fluoro-4-[({1′-[(3-{[(2S)-oxetan-2-yl]methyl}-6-(5-oxo-4,5-dihydro-1,2,4-oxadiazol-3-yl)-3H-imidazo[4,5-b]pyridin-2-yl)methyl]-1′,2′,3′,6′-tetrahydro-[2,4′-bipyridin]-6-yl}oxy)methyl]benzonitrile (Compound 322a) (7.5 mg, yield: 19%) as white solid.

MS Calcd: 594.2; MS Found: 595.2 [M+H]⁺.

¹H NMR (400 MHz, CD₃OD) δ 8.74 (d, J=1.6 Hz, 1H), 8.36 (s, 1H), 7.59-7.50 (m, 2H), 7.49-7.42 (m, 2H), 6.97 (d, J=7.6 Hz, 1H), 6.64 (d, J=8.4 Hz, 1H), 6.58 (br.s, 1H), 5.44 (s, 2H), 5.20-5.12 (m, 1H), 4.85-4.73 (m, 1H), 4.69-4.62 (m, 1H), 4.54-4.48 (m, 1H), 4.35-4.25 (m, 1H), 4.18-4.08 (m, 2H), 3.28 (br.s, 2H), 2.84 (t, J=5.2 Hz, 2H), 2.70-2.60 (m, 1H), 2.60-2.50 (m, 2H), 2.48-2.36 (m, 1H). ¹⁹F-NMR (377 MHz): −117.44.

### Example 165: 6-[(4-chloro-2-fluorophenyl)methoxy]-1′-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1′,2′,3′,6′-tetrahydro-2,4′-bipyridine (Compound 323a)

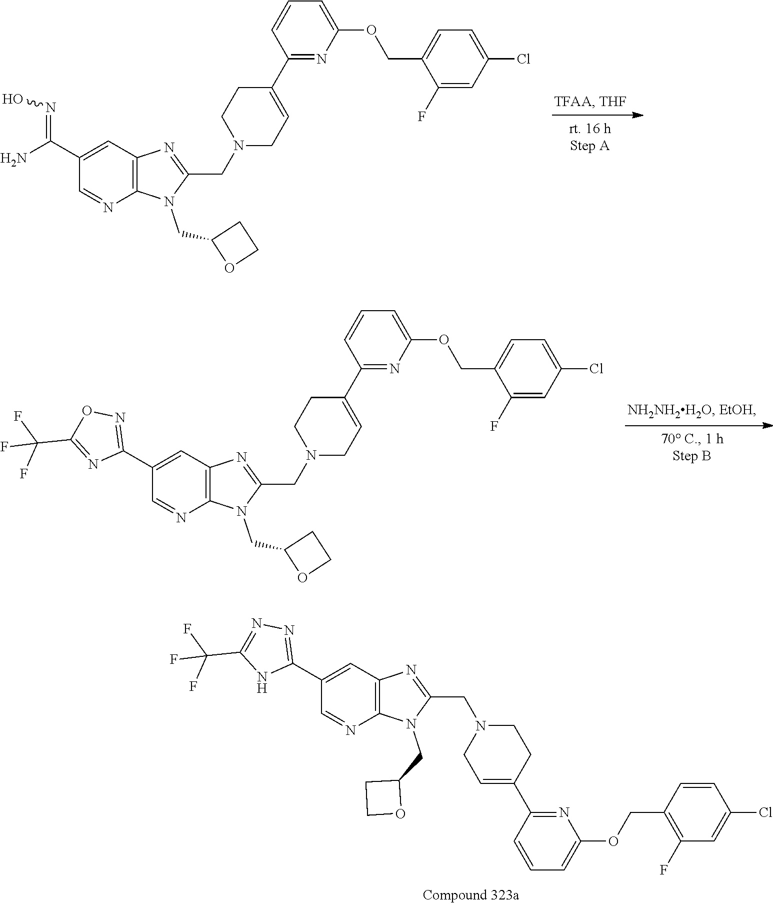

### Step A: (S)-3-(2-((6-((4-chloro-2-fluorobenzyl)oxy)-3′,6′-dihydro-[2,4′-bipyridin]-1′(2′H)-yl)methyl)-3-(oxetan-2-ylmethyl)-3H-imidazo[4,5-b]pyridin-6-yl)-5-(trifluoromethyl)-1,2,4-oxadiazole

To a solution of (S)-2-((6-((4-chloro-2-fluorobenzyl)oxy)-3′,6′-dihydro-[2,4′-bipyridin]-1′(2′H)-yl)methyl)-N′-hydroxy-3-(oxetan-2-ylmethyl)-3H-imidazo[4,5-b]pyridine-6-carboximidamide (53 mg) in THE (5 mL) were added dropwise TFAA (96 mg, 0.46 mmol) in THE (1 mL) at 0° C. The mixture was stirred at room temperature for 16 hours. The mixture was purified by prep-HPLC to obtain (S)-3-(2-((6-((4-chloro-2-fluorobenzyl)oxy)-3′,6′-dihydro-[2,4′-bipyridin]-1′(2′H)-yl)methyl)-3-(oxetan-2-ylmethyl)-3H-imidazo[4,5-b]pyridin-6-yl)-5-(trifluoromethyl)-1,2,4-oxadiazole (30 mg, yield: 50%) as white solid. MS Calcd.: 655.2; MS Found: 656.2 [M+H]⁺.

### Step B: 6-[(4-chloro-2-fluorophenyl)methoxy]-1′-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1′,2′,3′,6′-tetrahydro-2,4′-bipyridine (Compound 323a)

A mixture of (S)-3-(2-((6-((4-chloro-2-fluorobenzyl)oxy)-3′,6′-dihydro-[2,4′-bipyridin]-1′(2′H)-yl)methyl)-3-(oxetan-2-ylmethyl)-3H-imidazo[4,5-b]pyridin-6-yl)-5-(trifluoromethyl)-1,2,4-oxadiazole (30 mg, 0.046 mmol) and hydrazine hydrate (6 drops) in EtOH (3 mL) was stirred at 70° C. for 1 hour. The mixture was purified by prep-HPLC to obtain 6-[(4-chloro-2-fluorophenyl)methoxy]-1′-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1′,2′,3′,6′-tetrahydro-2,4′-bipyridine (Compound 323a) (11 mg, yield: 38%) as white solid.

MS Calcd: 654.2; MS Found: 655.2 [M+H]⁺.

¹H NMR (400 MHz, CD₃OD) δ 9.05 (d, J=1.6 Hz, 1H), 8.63 (s, 1H), 7.61 (t, J=8.0 Hz, 1H), 7.48 (t, J=8.0 Hz, 1H), 7.23-7.16 (m, 2H), 7.05 (d, J=7.6 Hz, 1H), 6.72 (br.s, 1H), 6.67 (d, J=8.0 Hz, 1H), 5.42 (s, 2H), 5.35-5.25 (m, 1H), 4.99-4.92 (m, 1H), 4.82-4.76 (m, 1H), 4.65-4.58 (m, 1H), 4.48-4.40 (m, 1H), 4.25-4.12 (m, 2H), 3.35 (br.s, 2H), 2.90 (t, J=6.4 Hz, 2H), 2.82-2.70 (m, 1H), 2.70-2.63 (m, 2H), 2.60-2.49 (m, 1H). ¹⁹F-NMR (377 MHz): −66.59, −117.70.

### Example 166: 3-fluoro-4-[({1′-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1′,2′,3′,6′-tetrahydro-[2,4′-bipyridin]-6-yl}oxy)methyl]benzonitrile (Compound 321a)

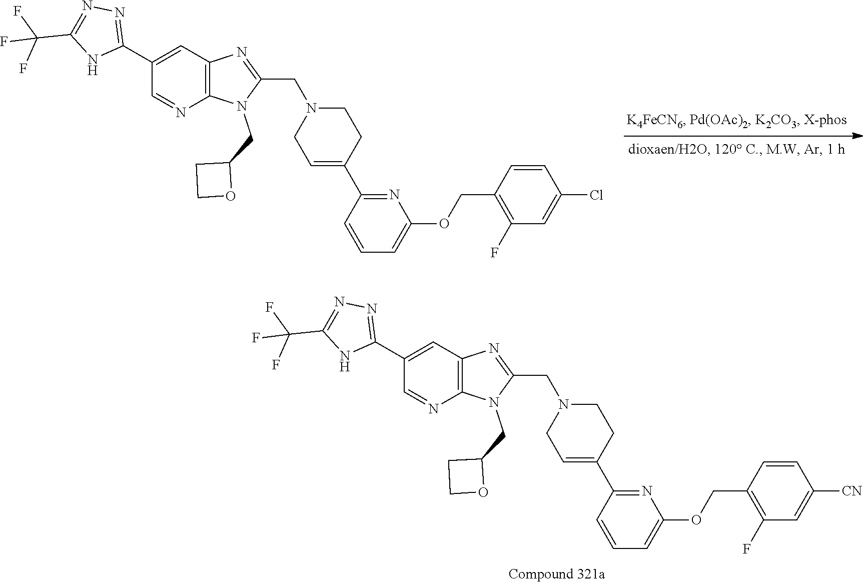 [SMILES: Fc1cc(Cl)ccc1COc1cccc(C2=CCN(Cc3nc4cc(-c5nnc(C(F)(F)F)[nH]5)cnc4n3C[C@@H]3CCO3)CC2)n1 ; N#Cc1ccc(COc2cccc(C3=CCN(Cc4nc5cc(-c6nnc(C(F)(F)F)[nH]6)cnc5n4C[C@@H]4CCO4)CC3)n2)c(F)c1]

A mixture of (S)-2-((6-((4-chloro-2-fluorobenzyl)oxy)-3′,6′-dihydro-[2,4′-bipyridin]-1′(2′H)-yl)methyl)-3-(oxetan-2-ylmethyl)-6-(5-(trifluoromethyl)-4H-1,2,4-triazol-3-yl)-3H-imidazo[4,5-b]pyridine (60 mg, 0.092 mmol), K₄Fe(CN)₆ (39 mg, 0.092 mmol), Pd(OAc)₂ (2 mg, 0.0092 mmol), Xphos (9 mg, 0.018 mmol) and K₂CO₃ (38 mg, 0.28 mmol) in 1,4-dioxane/H₂O (1 mL/0.2 mL) was stirred at 120° C. with M.W under Ar for 1 hours. The mixture was filtered and the filtrate was purified by prep-HPLC to obtain 3-fluoro-4-[({1′-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1′,2′,3′,6′-tetrahydro-[2,4′-bipyridin]-6-yl}oxy)methyl]benzonitrile (Compound 321a) (10 mg, yield: 18%) as white solid.

MS Calcd: 645.2; MS Found: 646.2 [M+H]⁺.

¹H NMR (400 MHz, CD₃OD) δ 9.05 (s, 1H), 8.64 (s, 1H), 7.70-7.60 (m, 2H), 7.55 (t, J=11.6 Hz, 2H), 7.06 (d, J=6.8 Hz, 1H), 6.72 (d, J=8.4 Hz, 1H), 6.68 (br.s, 1H), 5.53 (s, 2H), 5.33-5.23 (m, 1H), 4.97-4.73 (m, 4H), 4.64-4.58 (m, 1H), 4.46-4.38 (m, 1H), 4.25-4.13 (m, 2H), 2.91-2.84 (m, 2H), 2.83-2.73 (m, 1H), 2.64-2.58 (m, 2H), 2.57-2.48 (m, 1H). ¹⁹F-NMR (377 MHz): −66.10, −117.45.

### Example 167: 2-[(4-chloro-2-fluorophenyl)methoxy]-6-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}pyridine (Compound 327a)

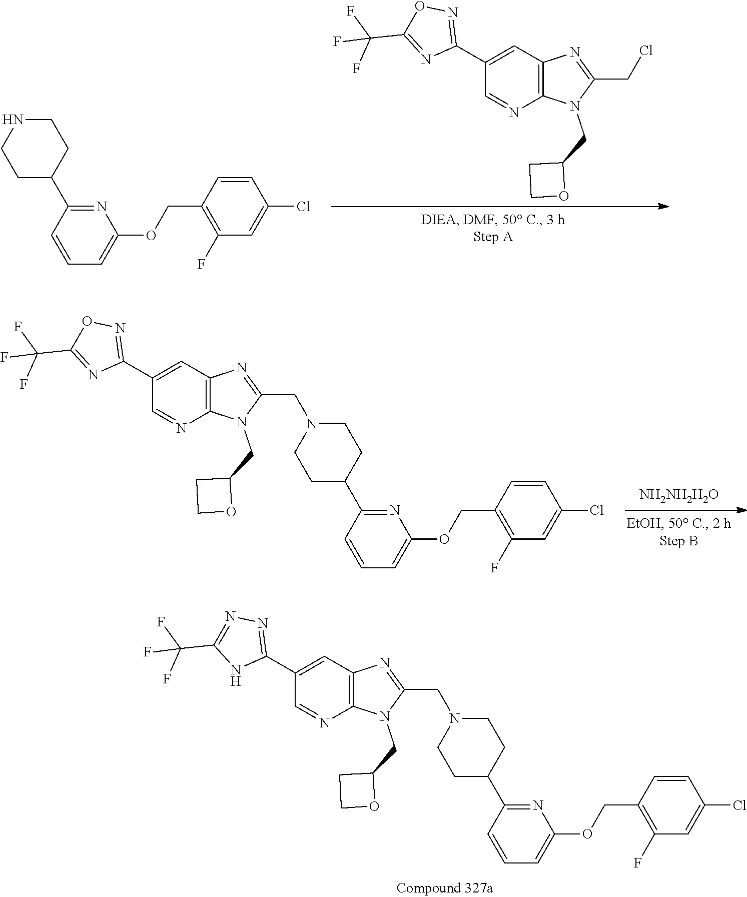

### Step A: (S)-3-(2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridin-6-yl)-5-(trifluoromethyl)-1,2,4-oxadiazole

A mixture of 2-((4-chloro-2-fluorobenzyl)oxy)-6-(piperidin-4-yl)pyridine hydrochloride (46 mg, 0.13 mmol) and (S)-3-(2-(chloromethyl)-3-(oxetan-2-ylmethyl)-3H-imidazo[4,5-b]pyridin-6-yl)-5-(trifluoromethyl)-1,2,4-oxadiazole (50 mg, 0.13 mmol), DIEA (50 mg, 0.39 mmol) in DMF (1 mL) was stirred at 50° C. for 3 hours. After the reaction was completed, the mixture was concentrated in vacuum. The residue was purified by column chromatography (PE/EA=1/1) to give (S)-3-(2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridin-6-yl)-5-(trifluoromethyl)-1,2,4-oxadiazole (68 mg, yield: 77%) as colorless oil. MS Calcd.: 657.2; MS Found: 658.4[M+H]⁺.

### Step B: 2-[(4-chloro-2-fluorophenyl)methoxy]-6-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}pyridine (Compound 327a)

A mixture of (S)-3-(2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridin-6-yl)-5-(trifluoromethyl)-1,2,4-oxadiazole (68 mg, 0.10 mmol) and NH₂NH₂·H₂O (16 mg, 0.30 mmol) in EtOH (1 mL) was stirred at 50° C. for 2 hours. After the reaction was completed. The reaction mixture was purified directly by prep-HPLC to give (S)-3-(2-((4-(6-((4-chloro-2-fluorobenzyl)oxy)pyridin-2-yl)piperidin-1-yl)methyl)-3-(oxetan-2-ylmethyl)-3H-imidazo[4,5-b]pyridin-6-yl)-5-(trifluoromethyl)-1,2,4-oxadiazole Compound 327a (26 mg, yield: 39%) as a white solid.

MS Calcd.: 656.2; MS Found: 657.3 [M+H]⁺.

¹H NMR (400 MHz, DMSO-d₆): δ 8.98 (d, J=2.0 Hz, 1H), 8.52 (d, J=2.0 Hz, 1H), 7.63 (dd, J=8.0 Hz, 7.2 Hz, 1H), 7.57 (t, J=8.0 Hz, 1H), 7.46 (dd, J=10.0 Hz, 2.0 Hz, 1H), 7.29 (dd, J=8.0 Hz, 1.6 Hz, 1H), 6.88 (d, J=7.2 Hz, 1H), 6.67 (d, J=8.0 Hz, 1H), 5.37 (s, 2H), 5.18-5.25 (m, 1H), 4.81-4.89 (m, 1H), 4.70-4.76 (m, 1H), 4.46-4.52 (m, 1H), 4.36-4.43 (m, 1H), 3.89-4.00 (m, 2H), 2.90-3.05 (m, 2H), 2.55-2.75 (m, 2H), 2.46-2.55 (m, 1H), 2.18-2.32 (m, 2H), 1.67-1.85 (m, 4H). ¹⁹F-NMR (377 MHz): −62.34, −115.21.

### Example 168: 3-fluoro-4-{[(6-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}pyridin-2-yl)oxy]methyl}benzonitrile (Compound 325a)

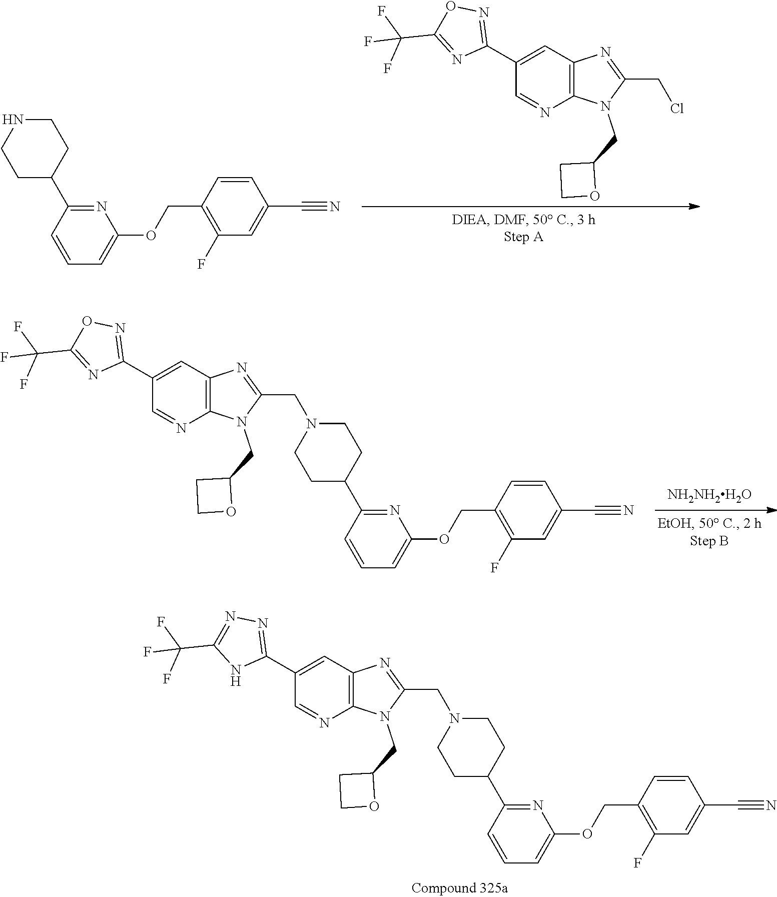

### Step A: (S)-3-fluoro-4-(((6-(1-((3-(oxetan-2-ylmethyl)-6-(5-(trifluoromethyl)-1,2,4-oxadiazol-3-yl)-3H-imidazo[4,5-b]pyridin-2-yl)methyl)piperidin-4-yl)pyridin-2-yl)oxy)methyl)benzonitrile

A mixture of 3-fluoro-4-(((6-(piperidin-4-yl)pyridin-2-yl)oxy)methyl)benzonitrile (45 mg) and DIEA (50 mg, 0.39 mmol) in DMF (1 mL) was stirred at room temperature for 10 minutes. The mixture was added with (S)-3-(2-(chloromethyl)-1-(oxetan-2-ylmethyl)-1H-benzo[d]imidazol-5-yl)-5-(trifluoromethyl)-1,2,4-oxadiazole (50 mg, 0.13 mmol) and stirred at 50° C. for 3 hours. After the reaction was completed, the mixture was diluted with EA (20 mL), washed with H₂O (10 ml×3). The organic layer was dried over anhydrous sodium sulfate, filtered and concentrated in vacuum. The residue was purified by column chromatography (PE/EA=1/1) to give (S)-3-fluoro-4-(((6-(1-((3-(oxetan-2-ylmethyl)-6-(5-(trifluoromethyl)-1,2,4-oxadiazol-3-yl)-3H-imidazo[4,5-b]pyridin-2-yl)methyl)piperidin-4-yl)pyridin-2-yl)oxy)methyl)benzonitrile (70 mg, yield: 75%) as a yellow solid. MS Calcd: 648.2; MS Found: 649.6 [M+H]⁺.

### Step B: 3-fluoro-4-{[(6-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}pyridin-2-yl)oxy]methyl}benzonitrile (Compound 325a)

A mixture of (S)-3-fluoro-4-(((6-(1-((3-(oxetan-2-ylmethyl)-6-(5-(trifluoromethyl)-1,2,4-oxadiazol-3-yl)-3H-imidazo[4,5-b]pyridin-2-yl)methyl)piperidin-4-yl)pyridin-2-yl)oxy)methyl)benzonitrile (70 mg, 0.10 mmol) and NH₂NH₂·H₂O (16 mg, 0.31 mmol) in EtOH (1 mL) was stirred at 50° C. for 2 hours. After the reaction was completed. The reaction mixture was purified by Prep-HPLC to give 3-fluoro-4-{[(6-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}pyridin-2-yl)oxy]methyl}benzonitrile (Compound 325a) (9.1 mg, yield: 13%) as a white solid.

MS Calcd.: 647.2; MS Found: 648.4 [M+H]⁺.

¹H NMR (400 MHz, DMSO-d₆): δ 9.00 (d, J=2.0 Hz, 1H), 8.59 (d, J=1.6 Hz, 1H), 7.89 (d, J=10.0 Hz, 1H), 7.62-7.73 (m, 3H), 6.89 (d, J=7.2 Hz, 1H), 6.72 (d, J=8.4 Hz, 1H), 5.47 (s, 2H), 5.18-5.25 (m, 1H), 4.84-4.90 (m, 1H), 4.70-4.77 (m, 1H), 4.47-4.52 (m, 1H), 4.35-4.41 (m, 1H), 3.91-4.01 (m, 2H), 2.89-3.01 (m, 2H), 2.66-2.75 (m, 1H), 2.56-2.65 (m, 1H), 2.45-2.55 (m, 1H), 2.19-2.32 (m, 2H), 1.64-1.82 (m, 4H). ¹⁹F-NMR (377 MHz): −63.61, −115.51.

### Example 169: 3-[2-({4-[(2S)-2-(4-chloro-2-fluorophenyl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one (Compound 375a)

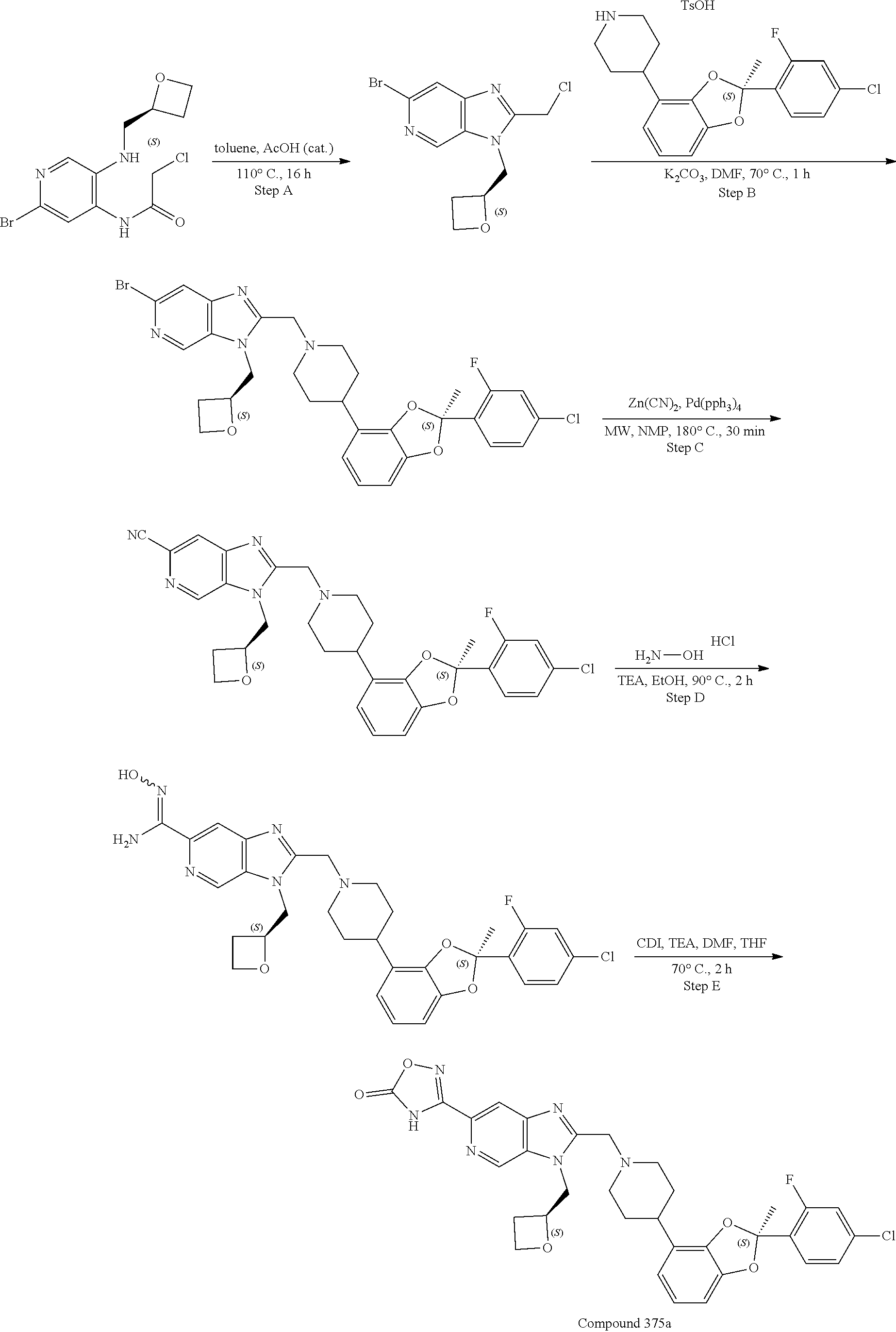

### Step A: (S)-6-bromo-2-(chloromethyl)-3-(oxetan-2-ylmethyl)-3H-imidazo[4,5-c]pyridine

A mixture of (S)—N-(2-bromo-5-((oxetan-2-ylmethyl)amino)pyridin-4-yl)-2-chloroacetamide (1.5 g, 4.5 mmol) in toluene (20 mL) and AcOH (0.2 mL) was heated to 110° C. for 16 hours. After cooled down to room temperature, the reaction mixture was quenched with Sat. aqueous NaHCO₃ (1 mL), diluted with water (10 mL). The organic layer was separated and evaporated in vacuo to give (S)-6-bromo-2-(chloromethyl)-3-(oxetan-2-ylmethyl)-3H-imidazo[4,5-c]pyridine (1.1 g, 85% purity, 65% yield) as a brown solid. The crude product was used in the next step directly without purification. MS Calcd.: 315.0; MS Found: 316.0 [M+H]⁺.

### Step B: 6-bromo-2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine

To a solution of (S)-4-(2-(4-chloro-2-fluorophenyl)-2-methylbenzo[d][1,3]dioxol-4-yl)piperidine 4-methylbenzenesulfonate (1.64 g) in DMF (15 mL) was added K₂CO₃ (1.31 g, 9.5 mmol). After 5 minutes, (S)-6-bromo-2-(chloromethyl)-3-(oxetan-2-ylmethyl)-3H-imidazo[4,5-c]pyridine (1.0 g, 3.2 mmol) was added. The resulting mixture was heated to 70° C. for 1 hour. The reaction was quenched with water, extracted with ethyl acetate (50 mL×3). The organic layers were combined, dried over Na₂SO₄, filtered and concentrated under vacuum. The residue was applied on a silica gel column and eluted with DCM:MeOH=20:1 to give 6-bromo-2-((4-((S)-2-(4-chloro-2-fluorophenyl)-2-methylbenzo[d][1,3] dioxol-4-yl)piperidin-1-yl)methyl)-3-(((S)-oxetan-2-yl)methyl)-3H-imidazo[4,5-c]pyridine (1.5 g, 77% yield) as a brown solid. MS Calcd.: 626.1; MS Found: 627.1 [M+H]⁺.

### Step C: 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile

To a solution of 6-bromo-2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine (470 mg, 0.75 mmol) in NMP (8 mL) was added Zn(CN)₂ (53 mg, 0.45 mmol) and Pd(PPh₃)₄ (130 mg, 0.11 mmol). The reaction mixture was irradiated with microwave radiation for 30 minutes at 180° C. The reaction was quenched with water, extracted with ethyl acetate (20 mL×3). The organic layers were combined, dried over Na₂SO₄, filtered and concentrated under vacuum. The residue was applied on a silica gel column and eluted with DCM:MeOH=25:1 to give 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (380 mg, 80% purity, 70% yield) as brown oil. MS Calcd.: 573.2; MS Found: 574.2 [M+H]⁺.

### Step D: 2-((4-((S)-2-(4-chloro-2-fluorophenyl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide

To a solution of hydroxylamine hydrochloride (49 mg, 0.70 mmol) in EtOH (6 mL) was added TEA (106 mg, 1.0 mmol). After a few minutes, 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (200 mg, 0.35 mmol) was added. The resulting mixture was heated to 90° C. for 2 hours. The reaction was cooled to room temperature. The solid was collected by filtration and dried to give 2-((4-((S)-2-(4-chloro-2-fluorophenyl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (150 mg) as a white solid. MS Calcd.: 606.2; MS Found: 607.2 [M+H]⁺.

### Step E: 3-[2-({4-[(2S)-2-(4-chloro-2-fluorophenyl)-2-methyl-2H-1,3-benzodioxol-4-yl]piperidin-1-yl}methyl)-3-{1[(2S)-oxetan-2-yl]methyl}-3H-imidazo[4,5-c]pyridin-6-yl]-4,5-dihydro-1,2,4-oxadiazol-5-one (Compound 375a)

To a solution of 2-((4-((S)-2-(4-chloro-2-fluorophenyl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (40 mg) in DMF (1 mL) and THE (1 mL) was added CDI (21 mg, 0.13 mmol) and TEA (20 mg, 0.20 mmol). The solution was stirred at 70° C. for 2 hours. The solvent was removed in vacuo. The residue was purified by Prep-HPLC to afford 3-[2-({4-[(2S)-2-(4-chloro-2-fluorophenyl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one (Compound 375a) (5.2 mg, yield: 12%) as a white solid.

MS Calcd.: 632.2; MS Found: 633.3 [M+H]⁺.

¹H NMR (400 MHz, DMSO-d₆): δ 9.13 (s, 1H), 8.14 (s, 1H), 7.50-7.60 (m, 2H), 7.33 (dd, J=8.4 Hz, 1.6 Hz, 1H), 6.70-6.80 (m, 3H), 5.13-5.20 (m, 1H), 4.87-4.93 (m, 1H), 4.70-4.77 (m, 1H), 4.45-4.53 (m, 1H), 4.38-4.44 (m, 1H), 4.01 (d, J=13.6 Hz, 1H), 3.85 (d, J=13.6 Hz, 1H), 3.00-3.07 (m, 1H), 2.84-2.91 (m, 1H), 2.60-2.77 (m, 2H), 2.41-2.53 (m, 1H), 2.15-2.35 (m, 2H), 2.02 (s, 3H), 1.65-1.83 (m, 4H). ¹⁹F-NMR (377 MHz): −110.78.

### Example 170: 4-[(2S)-2-(4-chloro-2-fluorophenyl)-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 366a)

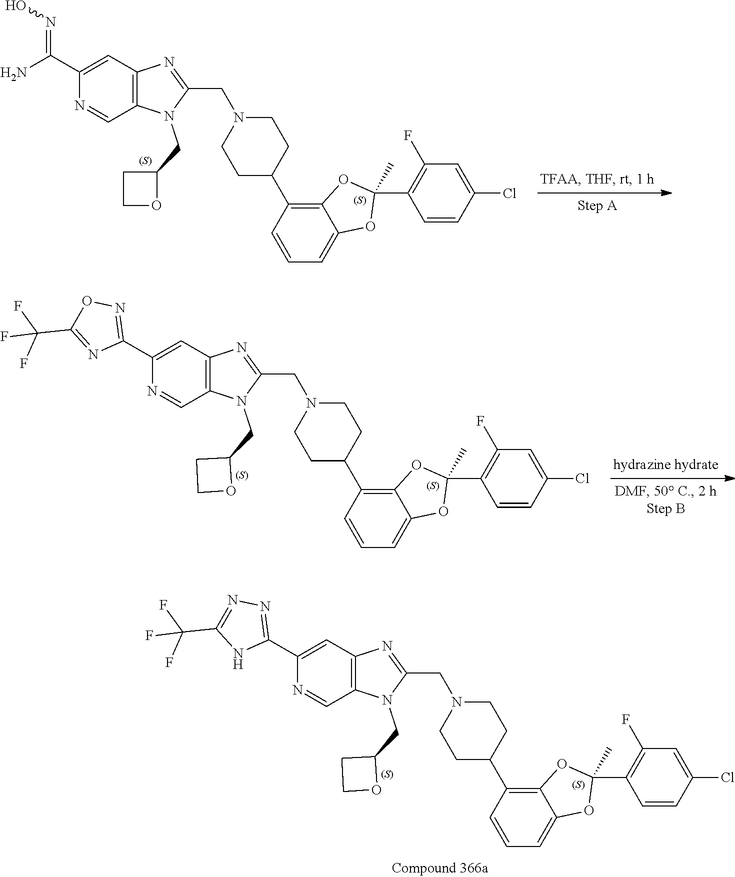

### Step A: 3-(2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole

To a solution of 2-((4-((S)-2-(4-chloro-2-fluorophenyl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (50 mg) in THF (1.5 mL) was added TFAA (69 mg, 0.33 mmol). The solution was stirred at room temperature for 1 hour. The reaction was quenched with sodium bicarbonate aqueous solution, extracted with ethyl acetate (20 mL×3). The organic layers were combined, dried over Na₂SO₄, filtered and concentrated under vacuum to give 3-(2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole (62 mg, 80% purity, 88% yield) as a brown solid. MS Calcd.: 684.2; MS Found: 685.3 [M+H]⁺.

### Step B: 4-[(2S)-2-(4-chloro-2-fluorophenyl)-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 366a)

To a solution of 3-(2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole (60 mg, 0.087 mmol) in DMF (1.5 mL) was added hydrazine hydrate (18 mg, 0.35 mmol). The solution was stirred at 50° C. for 2 hours. The reaction solution was filtered through the filter membrane, the filtrate was purified by Prep-HPLC to give 4-[(2S)-2-(4-chloro-2-fluorophenyl)-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 366a) (8.4 mg, yield: 14%) as a white solid.

MS Calcd.: 683.2; MS Found: 684.4 [M+H]⁺.

¹H NMR (400 MHz, DMSO-d₆): δ 9.17 (d, J=0.8 Hz, 1H), 8.29 (d, J=0.8 Hz, 1H), 7.52-7.60 (m, 2H), 7.34 (dd, J=8.4 Hz, 2.0 Hz, 1H), 6.73-6.82 (m, 3H), 5.15-5.22 (m, 1H), 4.88-4.97 (m, 1H), 4.73-4.80 (m, 1H), 4.48-4.53 (m, 1H), 4.40-4.47 (m, 1H), 4.02 (d, J=14.0 Hz, 1H), 3.85 (d, J=13.6 Hz, 1H), 3.00-3.08 (m, 1H), 2.87-2.91 (m, 1H), 2.60-2.80 (m, 2H), 2.43-2.51 (m, 1H), 2.18-2.34 (m, 2H), 2.03 (s, 3H), 1.68-1.82 (m, 4H). ¹⁹F-NMR (377 MHz): −63.68, −110.79.

### Example 171: 4-[(2S)-2-(4-chloro-2-fluorophenyl)-2-methyl-2H-1,3-benzodioxol-4-yl]-1-({6-[5-(difluoromethyl)-4H-1,2,4-triazol-3-yl]-3-{[(2S)-oxetan-2-yl]methyl}-3H-imidazo[4,5-c]pyridin-2-yl}methyl)piperidine (Compound 360a)

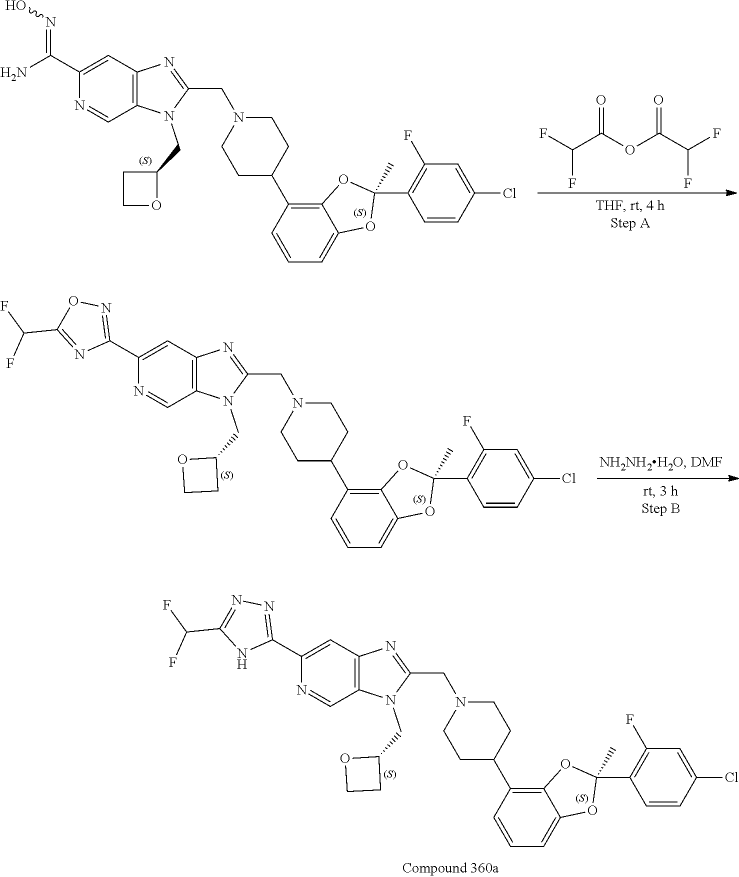

### Step A: 3-(2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(difluoromethyl)-1,2,4-oxadiazole

To a solution of 2-((4-((S)-2-(4-chloro-2-fluorophenyl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (60 mg) in THE (2 mL) was added 2,2-difluoroacetic anhydride (69 mg, 0.40 mmol). The solution was stirred at room temperature for 4 hours. The reaction was quenched with sodium bicarbonate aqueous solution, extracted with ethyl acetate (20 mL×3). The organic layers were combined, dried over Na₂SO₄, filtered and concentrated under vacuum. The residue was purified by Prep-TLC (DCM:MeOH=25:1) to give 3-(2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(difluoromethyl)-1,2,4-oxadiazole (30 mg, 45% yield) as a white solid. MS Calcd.: 666.2; MS Found: 667.2 [M+H]⁺.

### Step B: 4-[(2S)-2-(4-chloro-2-fluorophenyl)-2-methyl-2H-1,3-benzodioxol-4-yl]-1-({6-[5-(difluoromethyl)-4H-1,2,4-triazol-3-yl]-3-{[(2S)-oxetan-2-yl]methyl}-3H-imidazo[4,5-c]pyridin-2-yl}methyl)piperidine (Compound 360a)

To a solution of 3-(2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(difluoromethyl)-1,2,4-oxadiazole (30 mg, 0.045 mmol) in DMF (1.5 mL) was added hydrazine hydrate (9.0 mg, 0.18 mmol). The solution was stirred at room temperature for 3 hours. The reaction solution was filtered, the filtrate was purified by Prep-HPLC to give 4-[(2S)-2-(4-chloro-2-fluorophenyl)-2-methyl-2H-1,3-benzodioxol-4-yl]-1-({6-[5-(difluoromethyl)-4H-1,2,4-triazol-3-yl]-3-{[(2S)-oxetan-2-yl]methyl}-3H-imidazo[4,5-c]pyridin-2-yl}methyl)piperidine (Compound 360a) (6.6 mg, yield: 22%) as a white solid.

MS Calcd.: 665.2; MS Found: 666.4 [M+H]⁺.

¹H NMR (400 MHz, DMSO-d₆): δ 9.14 (s, 1H), 8.27 (d, J=0.8 Hz, 1H), 7.52-7.60 (m, 2H), 7.33 (dd, J=8.4 Hz, 1.6 Hz, 1H), 6.90-7.27 (m, 1H), 6.72-6.81 (m, 3H), 5.15-5.22 (m, 1H), 4.88-4.97 (m, 1H), 4.72-4.79 (m, 1H), 4.47-4.53 (m, 1H), 4.39-4.47 (m, 1H), 4.01 (d, J=14.0 Hz, 1H), 3.85 (d, J=14.0 Hz, 1H), 3.00-3.15 (m, 1H), 2.85-2.92 (m, 1H), 2.60-2.80 (m, 2H), 2.43-2.51 (m, 1H), 2.17-2.35 (m, 2H), 2.03 (s, 3H), 1.65-1.83 (m, 4H). ¹⁹F-NMR (377 MHz): −110.78, −115.73.

### Example 172: 4-[(2S)-2-(4-chloro-2-fluorophenyl)-2-methyl-2H-1,3-benzodioxol-4-yl]-1-{[6-(5-methyl-4H-1,2,4-triazol-3-yl)-3-{[(2S)-oxetan-2-yl]methyl}-3H-imidazo[4,5-c]pyridin-2-yl]methyl}piperidine (Compound 289a)

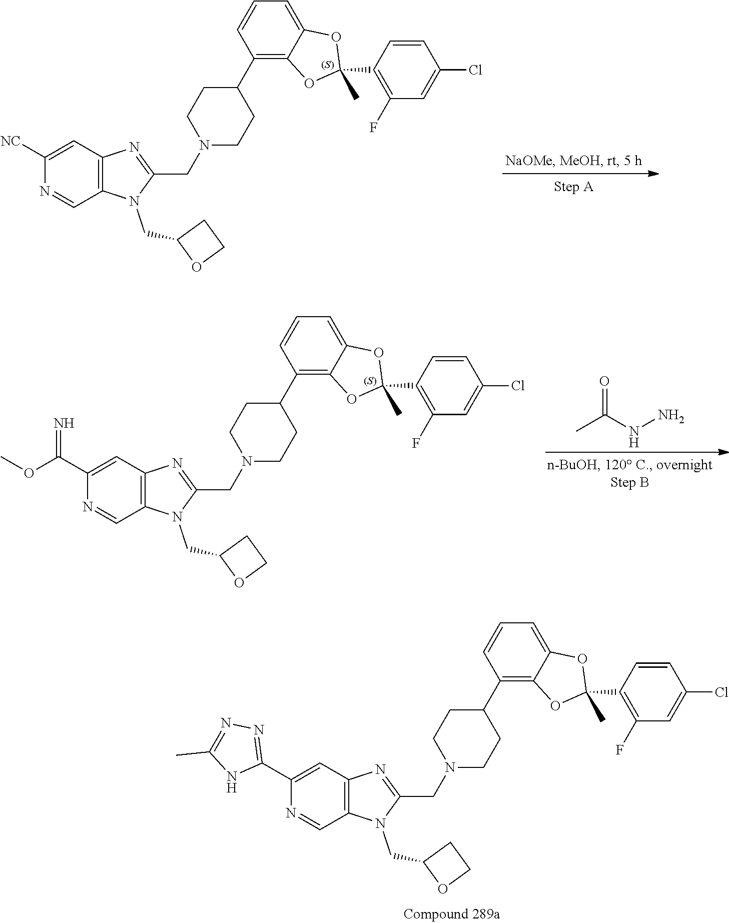

### Step A: methyl 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate

A mixture of 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (50 mg, 0.09 mmol), MeONa (47 mg, 0.9 mmol) in MeOH (2 mL) was stirred at room temperature for 5 h. After the reaction was completed, the mixture was concentrated in vacuum. The residue was purified by Prep-TLC (MeOH/DCM=1/20) to afford methyl 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (21 mg, yield: 42%) as a white solid. MS Calcd.: 605.2; MS Found: 606.3[M+H]⁺.

### Step B: 4-[(2S)-2-(4-chloro-2-fluorophenyl)-2-methyl-2H-1,3-benzodioxol-4-yl]-1-{[6-(5-methyl-4H-1,2,4-triazol-3-yl)-3-{[(2S)-oxetan-2-yl]methyl}-3H-imidazo[4,5-c]pyridin-2-yl]methyl}piperidine (Compound 289a)

A mixture of methyl 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (21 mg, 0.03 mmol) and acetohydrazide (5 mg, 0.07 mmol) in n-BuOH (2 mL) was stirred at 120° C. overnight. After the reaction was completed. The reaction mixture was purified by prep-HPLC to give 4-[(2S)-2-(4-chloro-2-fluorophenyl)-2-methyl-2H-1,3-benzodioxol-4-yl]-1-{[6-(5-methyl-4H-1,2,4-triazol-3-yl)-3-{[(2S)-oxetan-2-yl]methyl}-3H-imidazo[4,5-c]pyridin-2-yl]methyl}piperidine (Compound 289a) (3.0 mg, yield: 13%) as white solid.

MS Calcd.: 629.2; MS Found: 630.4 [M+H]⁺.

¹H NMR (400 MHz, DMSO-d₆): δ 9.07 (s, 1H), 8.17 (s, 1H), 7.52-7.60 (m, 2H), 7.34 (dd, J=8.0 Hz, 1.6 Hz, 1H), 6.72-6.80 (m, 3H), 5.13-5.20 (m, 1H), 4.82-4.91 (m, 1H), 4.68-4.76 (m, 1H), 4.38-4.53 (m, 2H), 3.99 (d, J=13.6 Hz, 1H), 3.83 (d, J=13.6 Hz, 1H), 3.00-3.07 (m, 1H), 2.84-2.91 (m, 1H), 2.60-2.79 (m, 2H), 2.40-2.51 (m, 1H), 2.15-2.40 (m, 5H), 2.03 (s, 3H), 1.67-1.81 (m, 4H). ¹⁹F-NMR (377 MHz): −110.79.

### Example 173: 4-{3-[(4-chloro-2-fluorophenyl)methoxy]phenyl}-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-1,2,3,6-tetrahydropyridine (Compound 307a)

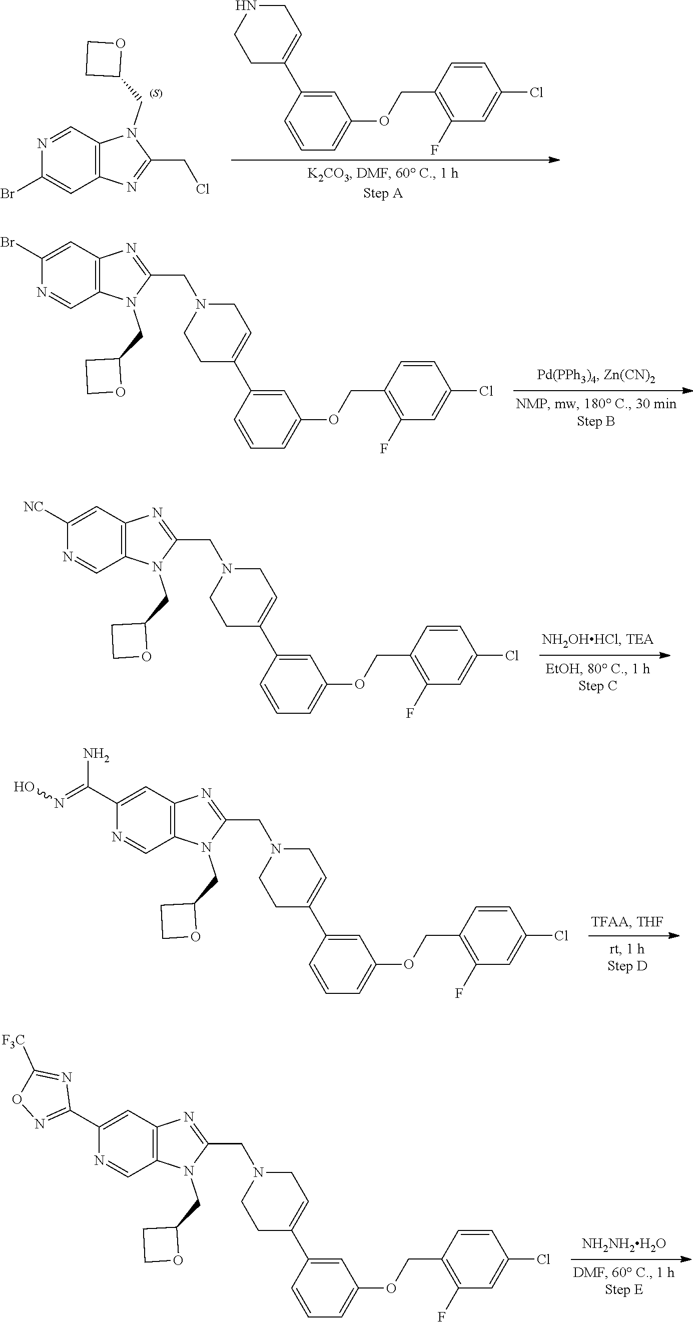

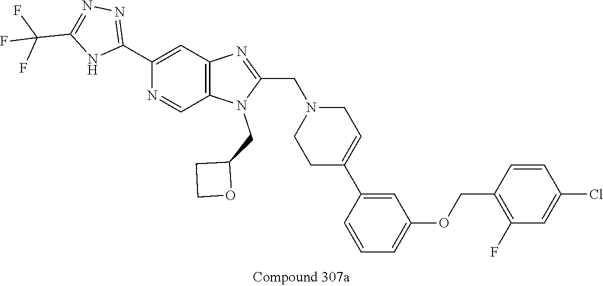 [SMILES: Fc1cc(Cl)ccc1COc1cccc(C2=CCN(Cc3nc4cc(-c5nnc(C(F)(F)F)[nH]5)ncc4n3C[C@@H]3CCO3)CC2)c1]

### Step A: (S)-6-bromo-2-((4-(3-((4-chloro-2-fluorobenzyl)oxy)phenyl)-3,6-dihydropyridin-1(2H)-yl)methyl)-3-(oxetan-2-ylmethyl)-3H-imidazo[4,5-c]pyridine

To a solution of (S)-6-bromo-2-(chloromethyl)-3-(oxetan-2-ylmethyl)-3H-imidazo[4,5-c]pyridine (500 mg, 1.6 mmol) in DMF (8.0 mL) was added K₂CO₃ (659 mg, 4.8 mmol) and 4-(3-((4-chloro-2-fluorobenzyl)oxy)phenyl)-1,2,3,6-tetrahydropyridine (1.03 g, 2.38 mmol). The resulting mixture was heated to 60° C. for 1 hour. The reaction was quenched with water, extracted with ethyl acetate. The organic layers were combined, dried over Na₂SO₄, filtered and concentrated under vacuum. The residue was purified by column chromatography on silica gel (DCM/MeOH=40/1) to give (S)-6-bromo-2-((4-(3-((4-chloro-2-fluorobenzyl)oxy)phenyl)-3,6-dihydropyridin-1(2H)-yl)methyl)-3-(oxetan-2-ylmethyl)-3H-imidazo[4,5-c]pyridine (600 mg, 63% yield) as yellow oil. MS Calcd.: 596.1; MS Found: 597.1 [M+H]⁺.

### Step B: (S)-2-((4-(3-((4-chloro-2-fluorobenzyl)oxy)phenyl)-3,6-dihydropyridin-1(2H)-yl)methyl)-3-(oxetan-2-ylmethyl)-3H-imidazo[4,5-c]pyridine-6-carbonitrile

To a mixture of (S)-6-bromo-2-((4-(3-((4-chloro-2-fluorobenzyl)oxy)phenyl)-3,6-dihydropyridin-1(2H)-yl)methyl)-3-(oxetan-2-ylmethyl)-3H-imidazo[4,5-c]pyridine (550 mg, 0.92 mmol) in NMP (6.0 mL) was added Zn(CN)₂ (65 mg, 0.55 mmol) and Pd(PPh₃)₄ (160 mg, 0.14 mmol). The reaction mixture was irradiated with microwave radiation for 30 minutes at 180° C. The reaction was quenched with water, extracted with ethyl acetate. The organic layers were combined, dried over Na₂SO₄, filtered and concentrated under vacuum. The residue was purified by column chromatography on silica gel (DCM/MeOH=40/1) to give (S)-2-((4-(3-((4-chloro-2-fluorobenzyl)oxy)phenyl)-3,6-dihydropyridin-1(2H)-yl)methyl)-3-(oxetan-2-ylmethyl)-3H-imidazo[4,5-c]pyridine-6-carbonitrile (180 mg, 36% yield) as yellow oil. MS Calcd.: 543.2; MS Found: 544.1 [M+H]⁺.

### Step C: (S)-2-((4-(3-((4-chloro-2-fluorobenzyl)oxy)phenyl)-3,6-dihydropyridin-1(2H)-yl)methyl)-N′-hydroxy-3-(oxetan-2-ylmethyl)-3H-imidazo[4,5-c]pyridine-6-carboximidamide

To a solution of (S)-2-((4-(3-((4-chloro-2-fluorobenzyl)oxy)phenyl)-3,6-dihydropyridin-1(2H)-yl)methyl)-3-(oxetan-2-ylmethyl)-3H-imidazo[4,5-c]pyridine-6-carbonitrile (180 mg, 0.33 mmol) in EtOH (4.0 mL) was added TEA (133 mg, 1.32 mmol) and NH₂OH HCl (46 mg, 0.66 mmol, 50% wt. in water). The resulting mixture was heated to 80° C. for 1 hour. The reaction was cooled to room temperature. The reaction was quenched with water, extracted with ethyl acetate. The organic layers were combined, dried over Na₂SO₄, filtered and concentrated to give crude (S)-2-((4-(3-((4-chloro-2-fluorobenzyl)oxy)phenyl)-3,6-dihydropyridin-1(2H)-yl)methyl)-N′-hydroxy-3-(oxetan-2-ylmethyl)-3H-imidazo[4,5-c]pyridine-6-carboximidamide (170 mg, 89% yield) as a white solid. MS Calcd.: 576.2; MS Found: 577.1 [M+H]⁺.

### Step D: (S)-3-(2-((4-(3-((4-chloro-2-fluorobenzyl)oxy)phenyl)-3,6-dihydropyridin-1(2H)-yl)methyl)-3-(oxetan-2-ylmethyl)-3H-imidazo[4,5-c]pyridin-6-yl)-5-(trifluoromethyl)-1,2,4-oxadiazole

To a solution of (S)-2-((4-(3-((4-chloro-2-fluorobenzyl)oxy)phenyl)-3,6-dihydropyridin-1(2H)-yl)methyl)-N′-hydroxy-3-(oxetan-2-ylmethyl)-3H-imidazo[4,5-c]pyridine-6-carboximidamide (110 mg, 0.19 mmol) in THE (3.0 mL) was added TFAA (120 mg, 0.57 mmol). The solution was stirred at room temperature for 1 hour. The reaction was quenched with sodium bicarbonate aqueous solution, extracted with ethyl acetate. The organic layers were combined, dried over Na₂SO₄, filtered and concentrated under vacuum, The residue was purified by column chromatography on silica gel (EA) to give (S)-3-(2-((4-(3-((4-chloro-2-fluorobenzyl)oxy)phenyl)-3,6-dihydropyridin-1(2H)-yl)methyl)-3-(oxetan-2-ylmethyl)-3H-imidazo[4,5-c]pyridin-6-yl)-5-(trifluoromethyl)-1,2,4-oxadiazole (90 mg, 72% yield) as yellow oil. MS Calcd.: 654.2; MS Found: 655.2 [M+H]⁺.

### Step E: 4-{3-[(4-chloro-2-fluorophenyl)methoxy]phenyl}-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-1,2,3,6-tetrahydropyridine (Compound 307a)

To a solution of (S)-3-(2-((4-(3-((4-chloro-2-fluorobenzyl)oxy)phenyl)-3,6-dihydropyridin-1(2H)-yl)methyl)-3-(oxetan-2-ylmethyl)-3H-imidazo[4,5-c]pyridin-6-yl)-5-(trifluoromethyl)-1,2,4-oxadiazole (90 mg, 0.14 mmol) in DMF (2.0 mL) was added hydrazine hydrate (18 mg, 0.55 mmol). The solution was stirred at 60° C. for 1 hour. The reaction solution was filtered, the filtrate was purified by Prep-HPLC (0.1% FA in H₂O, ACN) to give 4-{3-[(4-chloro-2-fluorophenyl)methoxy]phenyl}-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-1,2,3,6-tetrahydropyridine (Compound 307a) (4.43 mg, 4.9% yield) as a white solid.

MS Calcd.: 653.2; MS Found: 654.3 [M+H]⁺.

¹H NMR (400 MHz, DMSO-d₆): δ 9.14 (s, 1H), 8.28 (s, 1H), 7.59 (t, J=8.0 Hz, 1H), 7.47-7.51 (m, 1H), 7.30-7.35 (m, 1H), 7.26 (t, J=8.0 Hz, 1H), 7.00-7.08 (m, 2H), 6.89-6.93 (m, 1H), 6.16-6.21 (m, 1H), 5.08-5.17 (m, 3H), 4.85-4.95 (m, 1H), 4.70-4.78 (m, 1H), 4.45-4.55 (m, 1H), 4.37-4.43 (m, 1H), 4.11 (d, J=14.0 Hz, 1H), 3.98 (d, J=13.6 Hz, 1H), 3.17-3.22 (m, 2H), 2.67-2.80 (m, 4H), 2.30-2.52 (m, 2H). ¹⁹F-NMR (377 MHz): −63.30, −63.33, −63.37, −115.14.

### Example 174: 3-{2-[(4-{3-[(4-chloro-2-fluorophenyl)methoxy]phenyl}-1,2,3,6-tetrahydropyridin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 308a)

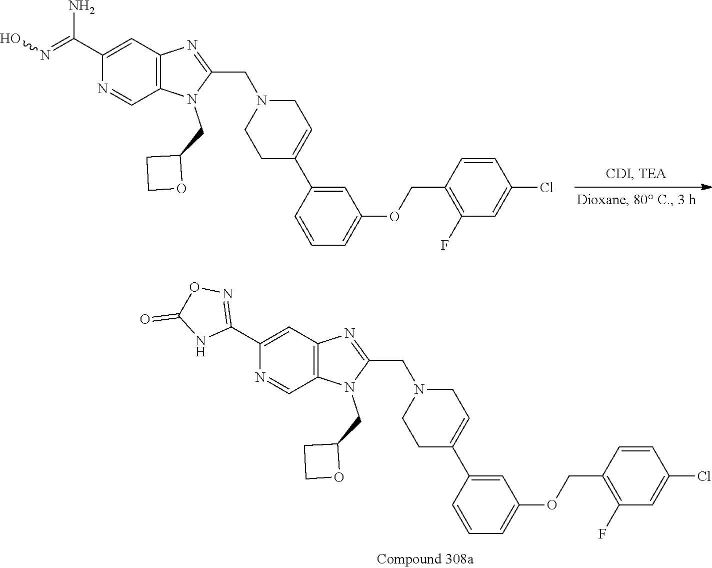 [SMILES: NC(=NO)c1cc2nc(CN3CC=C(c4cccc(OCc5ccc(Cl)cc5F)c4)CC3)n(C[C@@H]3CCO3)c2cn1 ; O=c1[nH]c(-c2cc3nc(CN4CC=C(c5cccc(OCc6ccc(Cl)cc6F)c5)CC4)n(C[C@@H]4CCO4)c3cn2)no1]

To a solution of (S)-2-((4-(3-((4-chloro-2-fluorobenzyl)oxy)phenyl)-3,6-dihydropyridin-1(2H)-yl)methyl)-N′-hydroxy-3-(oxetan-2-ylmethyl)-3H-imidazo[4,5-c]pyridine-6-carboximidamide (30 mg) in dioxane (3.0 mL) was added TEA (21 mg, 0.21 mmol) and CDI (17 mg, 0.104 mmol). The solution was stirred at 80° C. for 3 hours. The reaction mixture was directly purified by Prep-HPLC (0.1% FA in H₂O, ACN) to afford 3-{2-[(4-{3-[(4-chloro-2-fluorophenyl)methoxy]phenyl}-1,2,3,6-tetrahydropyridin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 308a) (5.9 mg, yield: 19%) as a white solid.

MS Calcd.: 602.2; MS Found: 603.2 [M+H]⁺.

¹H NMR (400 MHz, DMSO-d₆): δ 9.13 (s, 1H), 8.16 (s, 1H), 7.59 (t, J=8.4 Hz, 1H), 7.47-7.51 (m, 1H), 7.30-7.35 (m, 1H), 7.26 (t, J=8.0 Hz, 1H), 7.01-7.06 (m, 2H), 6.89-6.93 (m, 1H), 6.17-6.20 (m, 1H), 5.08-5.18 (m, 3H), 4.87-4.93 (m, 1H), 4.70-4.76 (m, 1H), 4.43-4.52 (m, 1H), 4.36-4.42 (m, 1H), 4.10 (d, J=13.6 Hz, 1H), 3.98 (d, J=13.6 Hz, 1H), 3.17-3.24 (m, 2H), 2.33-2.79 (m, 6H). ¹⁹F-NMR (377 MHz): −115.14.

### Example 175: 3-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 316a)

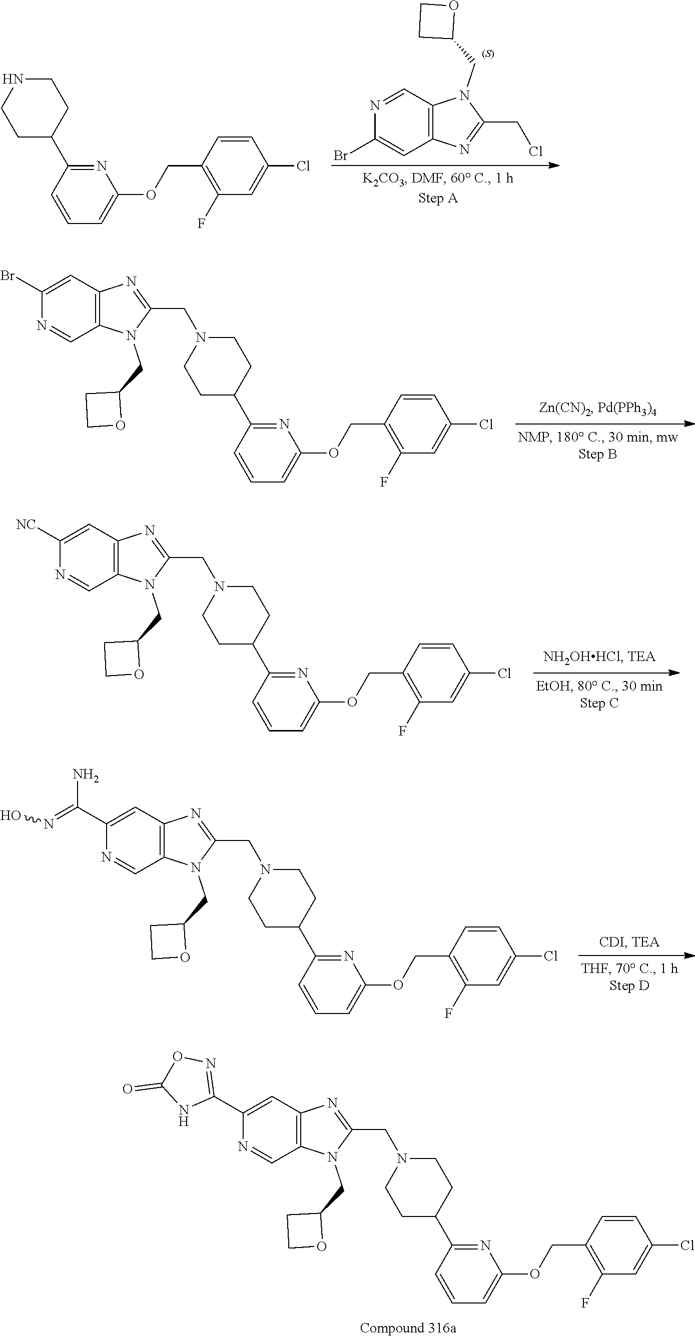

### Step A: (S′)-6-bromo-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine

A solution of 2-((4-chloro-2-fluorobenzyl)oxy)-6-(piperidin-4-yl)pyridine (640 mg, TFA salt) and K₂CO₃ (657 mg, 4.8 mmol) in DMF (5 mL) was stirred at room temperature for 2 mins. Then (S)-6-bromo-2-(chloromethyl)-3-(oxetan-2-ylmethyl)-3H-imidazo[4,5-c]pyridine (500 mg, 1.6 mmol.) was added. The reaction mixture was stirred at 60° C. for 1 hour. After the reaction was quenched with H₂O and extracted with DCM/MeOH=30/1. The organic layers were combined, dried over Na₂SO₄ and concentrated. The residue was purified by column chromatography on silica gel (DCM/MeOH=40/1) to give (S)-6-bromo-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine (790 mg, 66% yield) as a brown solid. MS Calcd.: 599.1; MS Found: 600.1 [M+H]*.

### Step B: (S)-2-((4-(6-((4-chloro-2-fluorobenzyloxy)pyridin-2-yl)piperidin-1-yl)methyl)-3-(oxetan-2 ylmethyl)-3H-imidazo[4,5-c]pyridine-6-carbonitrile

To a solution of (S)-6-bromo-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine (700 mg, 1.17 mmol) and Zn(CN)₂ (82.3 mg, 0.7 mmol) in NMP (15 mL) was added Pd(PPh₃)₄ (202 mg, 0.18 mmol). The reaction was stirred at 180° C. for 30 min under microwave. After the reaction was completed, the residue was quenched with H₂O and extracted with DCM/MeOH=30/1. The organic layers were combined, dried over Na₂SO₄ and concentrated. The residue was purified by silica gel column chromatography (DCM/MeOH=20/1) to give (S)-2-((4-(6-((4-chloro-2-fluorobenzyl)oxy)pyridin-2-yl)piperidin-1-yl)methyl)-3-(oxetan-2 ylmethyl)-3H-imidazo[4,5-c]pyridine-6-carbonitrile (140 mg, 22% yield) as yellow oil. MS Calcd.: 546.2; MS Found: 547.2 [M+H]⁺.

### Step C: (S)-2-((4-(6-((4-chloro-2-fluorobenzyl)oxy)pyridin-2-yl)piperidin-1-yl)methyl)-N′-hydroxy-3-(oxetan-2-ylmethyl)-3H-imidazo[4,5-c]pyridine-6-carboximidamide

A mixture of (S)-2-((4-(6-((4-chloro-2-fluorobenzyl)oxy)pyridin-2-yl)piperidin-1-yl)methyl)-3-(oxetan-2ylmethyl)-3H-imidazo[4,5-c]pyridine-6-carbonitrile (130 mg, 0.24 mmol) and TEA (120 mg, 1.2 mmol) in EtOH (2 mL) was added in NH₂OH HCl (49.6 mg, 0.71 mmol). The reaction was stirred at 80° C. for 30 mins. After the reaction was completed, the reaction was quenched with H₂O and extracted with DCM/MeOH=30/1. The organic layers were combined, dried over Na₂SO₄ and concentrated to give (S)-2-((4-(6-((4-chloro-2-fluorobenzyl)oxy)pyridin-2-yl)piperidin-1-yl)methyl)-N′-hydroxy-3-(oxetan-2-ylmethyl)-3H-imidazo[4,5-c]pyridine-6-carboximidamide (80 mg, 58% yield) as a white solid. MS Calcd.: 579.2; MS Found: 580.2 [M+H]⁺.

### Step D: 3-{2-[(4-{6-[(4-chloro-2-fluorophenyl)methoxy]pyridin-2-yl}piperidin-1-yl)methyl]-3-1{[(2S)-oxetan-2-yl]methyl}-3H-imidazo[4,5-c]pyridin-6-yl}-4,5-dihydro-1,2,4-oxadiazol-5-one (Compound 316a)

To a solution of (S)-2-((4-(6-((4-chloro-2-fluorobenzyl)oxy)pyridin-2-yl)piperidin-1-yl)methyl)-N′-hydroxy-3-(oxetan-2-ylmethyl)-3H-imidazo[4,5-c]pyridine-6-carboximidamide (50 mg) and TEA (35 mg, 0.35 mmol) in THE (2 mL) was added in CDI (42 mg, 0.26 mmol). The reaction was stirred at 70° C. for 1 h. The reaction was purified directly by Prep-HPLC to give 3-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 316a) (1.25 mg, yield: 2%) as a white solid.

MS Calcd.: 605.2; MS Found: 606.4 [M+H]⁺.

¹H NMR (400 MHz, DMSO-d₆): δ 9.29 (s, 1H), 8.28 (s, 1H), 7.70 (t, J=8.0 Hz, 1H), 7.60 (t, J=8.0 Hz, 1H), 7.49 (dd J=10.0 Hz, J=2.0 Hz, 1H), 7.32 (dd, J=8.4 Hz, J=2.0 Hz, 1H), 6.90-6.97 (m, 1H) 6.75 (d, J=8.4 Hz, 1H), 5.41 (s, 2H), 5.07-5.15 (m, 1H), 4.88-4.97 (m, 2H), 4.72-4.80 (m, 1H), 4.45-4.55 (m, 1H), 4.35-4.45 (m, 1H), 3.60-3.90 (m, 1H), 2.80-3.00 (m, 1H), 2.65-2.80 (m, 1H), 2.33-2.50 (m, 3H), 1.95-2.15 (m, 4H), 0.80-1.29 (m, 2H). ¹⁹F-NMR (377 MHz): −115.21.

### Example 176: 5-chloro-2-(2-methyl-4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}-2H-1,3-benzodioxol-2-yl)pyridine (Compound 344a)

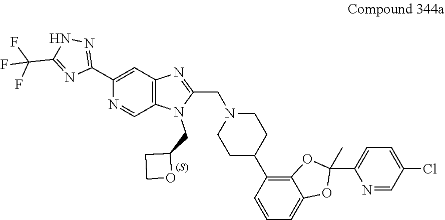

Starting from 5-chloro-2-(2-methyl-4-(piperidin-4-yl)benzo[d][1,3]dioxol-2-yl)pyridine, 5-chloro-2-(2-methyl-4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}-2H-1,3-benzodioxol-2-yl)pyridine (Compound 344a) (21 mg) was obtained with similar procedure of Compound 307a as a white solid.

MS Calcd.: 666.2; MS Found: 667.4 [M+H]⁺.

¹H NMR (400 MHz, DMSO-d₆): δ 9.15 (s, 1H), 8.70-8.73 (m, 1H), 8.28 (s, 1H), 8.01 (dt, J=8.4 Hz, 2.4 Hz, 1H), 7.61 (dd, J=8.8 Hz, 3.6 Hz, 1H), 6.72-6.82 (m, 3H), 5.13-5.21 (m, 1H), 4.87-4.95 (m, 1H), 4.70-4.78 (m, 1H), 4.39-4.52 (m, 2H), 4.01 (dd, J=14.0 Hz, 3.6 Hz, 1H), 3.84 (dd, J=13.6 Hz, 4.0 Hz, 1H), 3.02-3.08 (m, 1H), 2.84-2.90 (m, 1H), 2.60-2.79 (m, 2H), 2.40-2.51 (m, 1H), 2.15-2.33 (m, 2H), 2.01 (s, 3H), 1.68-1.83 (m, 4H). ¹⁹F-NMR (377 MHz): −63.61.

### Example 177: 3-[2-({4-[2-(5-chloropyridin-2-yl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one (Compound 343a)

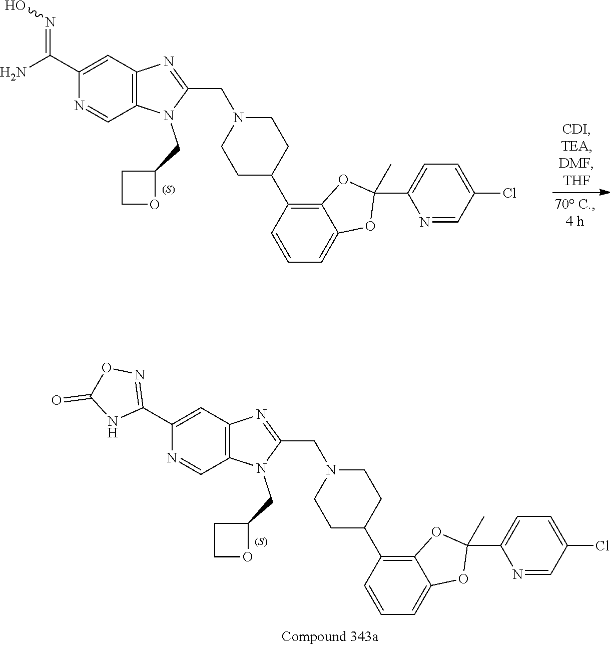

To a solution of 2-((4-(2-(5-chloropyridin-2-yl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (40 mg) in DMF (1 mL) and THE (1 mL) was added CDI (17 mg, 0.10 mmol) and TEA (21 mg, 0.20 mmol). The solution was stirred at 70° C. for 4 hours. The solvent was removed in vacuo. The residue was purified by Prep-HPLC to afford 3-[2-({4-[2-(5-chloropyridin-2-yl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one (Compound 343a) (11 mg, yield: 27%) as a white solid.

MS Calcd.: 615.2; MS Found: 616.3 [M+H]⁺.

¹H NMR (400 MHz, DMSO-d₆): δ 9.29 (s, 1H), 8.74 (d, J=2.0 Hz, 1H), 8.28 (s, 1H), 8.02 (dd, J=8.4 Hz, 2.4 Hz, 1H), 7.64 (d, J=8.4 Hz, 1H), 6.80-6.90 (m, 2H), 6.73-6.80 (m, 1H), 5.07-5.15 (m, 1H), 4.70-4.97 (m, 4H), 4.47-4.55 (m, 1H), 4.37-4.45 (m, 1H), 3.60-3.75 (m, 2H), 3.10-3.34 (m, 2H), 2.88-3.01 (m, 1H), 2.70-2.80 (m, 1H), 2.30-2.41 (m, 1H), 1.90-2.18 (m, 7H).

### Example 178: 4-[2-(4-chlorophenyl)-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 361a)

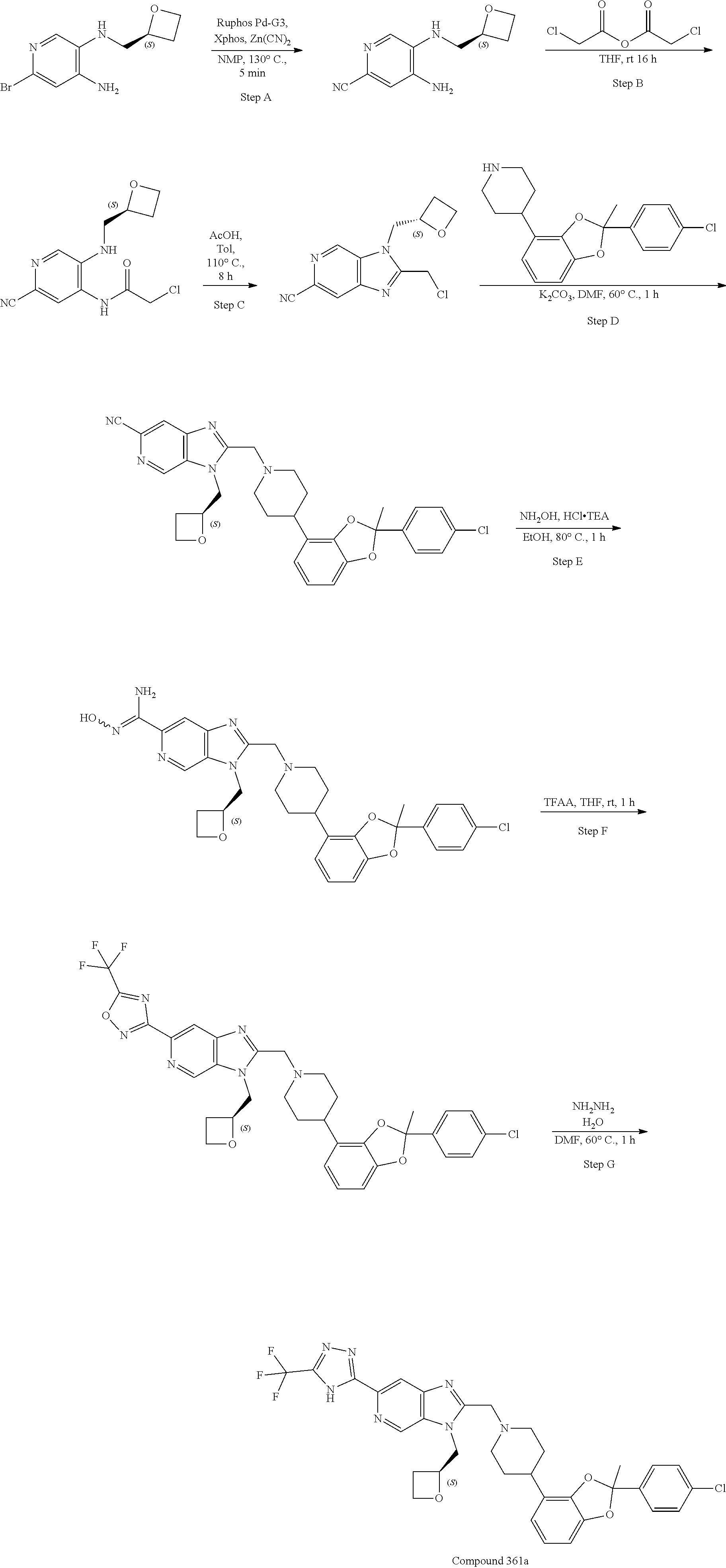

### Step A: (S)-4-amino-5-((oxetan-2-ylmethyl)amino)picolinonitrile

A mixture of (S)-6-bromo-N3-(oxetan-2-ylmethyl)pyridine-3,4-diamine (300 mg, 1.2 mmol), Ruphos Pd-G3 (195 mg, 0.23 mmol), X-phos (112 mg, 0.23 mmol) and Zn(CN)₂ (1.64 g, 14.0 mmol) in NMP (6.0 mL) was stirred at 130° C. for 5 min. After the reaction was completed, the mixture was filtered and diluted with EA (50 mL)/brine (50 mL). The organic layer was concentrated under vacuum. The residue was purified by column chromatography on silica gel (DCM/MeOH=30/1) to give (S)-4-amino-5-((oxetan-2-ylmethyl)amino)picolinonitrile (260 mg, crude) as colorless oil.

MS Calcd.: 204.1; MS Found: 205.2[M+H]⁺.

### Step B: (S)-2-chloro-N-(2-cyano-5-((oxetan-2-ylmethyl)amino)pyridin-4-yl)acetamide

A mixture of (S)-4-amino-5-((oxetan-2-ylmethyl)amino)picolinonitrile (260 mg, crude) and 2-chloroacetic anhydride (238 mg, 1.4 mmol) in THE (3.0 mL) was stirred at room temperature for 16 hours. The reaction mixture was directly purified by column chromatography on silica gel (PE/EA=1/3) to give (S)-2-chloro-N-(2-cyano-5-((oxetan-2-ylmethyl)amino)pyridin-4-yl)acetamide (200 mg, 56% yield) as a white solid. MS Calcd.: 280.1; MS Found: 281.1 [M+H]⁺.

### Step C: (S)-2-(chloromethyl)-3-(oxetan-2-ylmethyl)-3H-imidazo[4,5-c]pyridine-6-carbonitrile

A mixture of (S)-2-(chloromethyl)-3-(oxetan-2-ylmethyl)-3H-imidazo[4,5-c]pyridine-6-carbonitrile (180 mg, 0.64 mmol) in toluene (5.0 mL) and AcOH (0.2 mL) was heated to 110° C. for 8 hours. After the reaction was cooled to room temperature, NaHCO₃ (sat.) (4.0 mL) was added and extracted with ethyl acetate. The organic layers were combined, dried over Na₂SO₄, filtered and concentrated under vacuum. The residue was purified by column chromatography on silica gel (DCM/MeOH=50/1) to give (S)-2-(chloromethyl)-3-(oxetan-2-ylmethyl)-3H-imidazo[4,5-c]pyridine-6-carbonitrile (110 mg, 65% yield) as a yellow solid. MS Calcd.: 262.2; MS Found: 263.1 [M+H]⁺.

### Step D: 2-((4-(2-(4-chlorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile

To a solution of (S)-2-(chloromethyl)-3-(oxetan-2-ylmethyl)-3H-imidazo[4,5-c]pyridine-6-carbonitrile (110 mg, 0.42 mmol) in DMF (3.0 mL) was added K₂CO₃ (174 mg, 1.26 mmol) and 4-(2-(4-chlorophenyl)-2-methylbenzo[d][1,3]dioxol-4-yl)piperidine (203 mg, 0.46 mmol). The resulting mixture was heated to 60° C. for 1 hour. The reaction was quenched with water, extracted with ethyl acetate. The organic layers were combined, dried over Na₂SO₄, filtered and concentrated under vacuum. The residue was purified by column chromatography on silica gel (DCM/MeOH=40/1) to give 2-((4-(2-(4-chlorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (180 mg, 77% yield) as colorless oil. MS Calcd.: 555.2; MS Found: 556.2 [M+H]⁺.

### Step E: 2-((4-(2-(4-chlorophenyl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide

To a solution of 2-((4-(2-(4-chlorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (150 mg, 0.27 mmol) in EtOH (4.0 mL) was added TEA (218 mg, 2.16 mmol) and NH₂OH HCl (37 mg, 0.54 mmol). The resulting mixture was heated to 80° C. for 1 hour. The reaction was cooled to room temperature. The reaction was quenched with water, extracted with ethyl acetate. The organic layers were combined, dried over Na₂SO₄, filtered and concentrated to give crude 2-((4-(2-(4-chlorophenyl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (190 mg, ca.100% yield) as a white solid. MS Calcd.: 588.2; MS Found: 589.3 [M+H]⁺.

### Step F: 3-(2-((4-(2-(4-chlorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole

To a solution of 2-((4-(2-(4-chlorophenyl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (100 mg, 0.17 mmol) in THE (3.0 mL) was added TFAA (107 mg, 0.51 mmol). The solution was stirred at room temperature for 1 hour. The reaction was quenched with Sat. NaHCO₃, extracted with ethyl acetate. The organic layers were combined, dried over Na₂SO₄, filtered and concentrated under vacuum. The residue was purified by column chromatography on silica gel (EA) to give 3-(2-((4-(2-(4-chlorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole (80 mg, 71% yield) as colorless oil. MS Calcd.: 666.2; MS Found: 667.1 [M+H]⁺.

### Step G: 4-[2-(4-chlorophenyl)-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 361a)

To a solution of 3-(2-((4-(2-(4-chlorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole (80 mg, 0.12 mmol) in DMF (2.0 mL) was added hydrazine hydrate (12 mg, 0.36 mmol). The solution was stirred at 60° C. for 1 hour.

The reaction solution was filtered, the filtrate was purified by Prep-HPLC (0.1% HCOOH) to give 4-[2-(4-chlorophenyl)-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 361a) (17 mg, 21 yield) as a white solid.

MS Calcd.: 665.2; MS Found: 666.3 [M+H]⁺.

¹H NMR (400 MHz, DMSO-d₆): δ 9.16 (s, 1H), 8.28 (d, J=0.8 Hz, 1H), 7.57-7.61 (m, 2H), 7.47-7.51 (m, 2H), 6.70-6.80 (m, 3H), 5.15-5.23 (m, 1H), 4.88-4.97 (m, 1H), 4.72-4.79 (m, 1H), 4.39-4.53 (m, 2H), 3.98-4.06 (m, 1H), 3.80-3.90 (m, 1H), 3.00-3.08 (m, 1H), 2.85-2.92 (m, 1H), 2.60-2.80 (m, 2H), 2.45-2.51 (m, 1H), 2.17-2.35 (m, 2H), 1.97 (s, 3H), 1.72-1.82 (m, 4H). ¹⁹F-NMR (377 MHz): −63.62.

### Example 179: 3-[2-({4-[2-(4-chlorophenyl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one (Compound 347a)

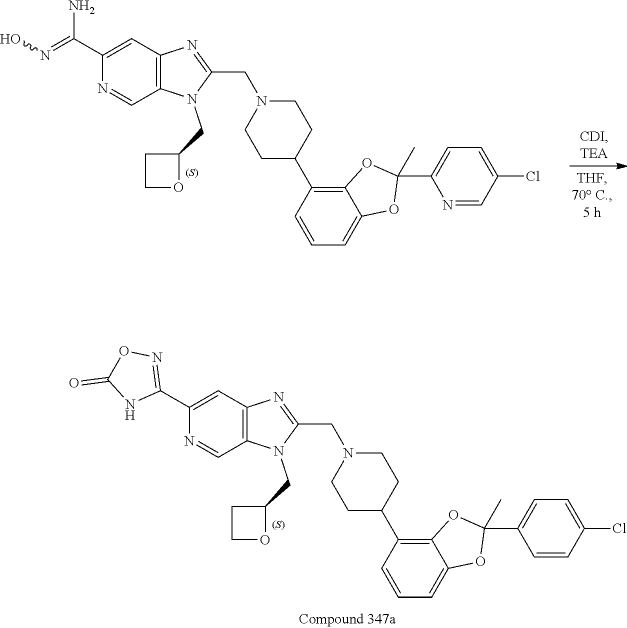

To a solution of 2-((4-(2-(4-chlorophenyl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (90 mg) in THF (3.0 mL) was added TEA (69 mg, 0.68 mmol) and CDI (55 mg, 0.34 mmol). The solution was stirred at 70° C. for 5 hours. The reaction mixture was directly purified by Prep-HPLC (0.1% TFA) to afford 3-[2-({4-[2-(4-chlorophenyl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one (Compound 347a) (12 mg, yield: 13%) as a white solid.

MS Calcd.: 614.2; MS Found: 615.2 [M+H]⁺.

¹H NMR (400 MHz, DMSO-d₆): δ 9.30 (s, 1H), 8.29 (s, 1H), 7.60-7.64 (m, 2H), 7.48-7.53 (m, 2H), 6.80-6.86 (m, 2H), 6.70-6.80 (m, 1H), 5.08-5.15 (m, 1H), 4.88-4.98 (m, 1H), 4.74-4.80 (m, 1H), 4.48-4.55 (m, 1H), 4.37-4.43 (m, 1H), 3.40-3.80 (m, 6H), 2.89-3.03 (m, 1H), 2.70-2.80 (m, 1H), 2.30-2.42 (m, 1H), 1.93-2.20 (m, 7H).

### Example 180: 3-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-b]pyridin-6-yl}-4,5-dihydro-1,2,4-oxadiazol-5-one (Compound 328a)

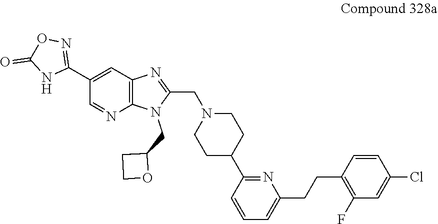 [SMILES: O=c1[nH]c(-c2cnc3c(c2)nc(CN2CCC(c4cccc(CCc5ccc(Cl)cc5F)n4)CC2)n3C[C@@H]2CCO2)no1]

3-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-b]pyridin-6-yl}-4,5-dihydro-1,2,4-oxadiazol-5-one (Compound 328a) (11 mg) as white solid.

MS Calcd.: 605.2; MS Found: 606.2 [M+H]⁺.

¹H NMR (400 MHz, DMSO-d₆): δ 8.78 (d, J=2.0 Hz, 1H), 8.38 (d, J=2.0 Hz, 1H), 7.63 (dd, J=8.0 Hz, J=7.6 Hz, 1H), 7.56 (t, J=8.0 Hz, 1H), 7.46 (dd, J=9.6 Hz, J=1.6 Hz, 1H), 7.29 (dd, J=8.4 Hz, J=2.0 Hz, 1H), 6.88 (d, J=7.2 Hz, 1H), 6.68 (d, J=8.0 Hz, 1H), 5.37 (s, 2H), 5.17-5.23 (m, 1H), 4.82-4.90 (m, 1H), 4.69-4.78 (m, 1H), 4.45-4.52 (m, 1H), 4.33-4.40 (m, 1H), 3.92-4.03 (m, 2H), 2.90-3.02 (m, 2H), 2.57-2.73 (m, 2H), 2.40-2.51 (m, 1H), 2.20-2.34 (m, 2H), 1.69-1.86 (m, 4H). ¹⁹F-NMR (377 MHz): −115.22.

### Example 181: 3-[2-({4-[2-(4-chlorophenyl)-2-methyl-2H-1,3-benzodioxol-4-yl]piperidin-1-yl}methyl)-3-{[(2S)-oxetan-2-yl]methyl}-3H-imidazo[4,5-b]pyridin-6-yl]-4,5-dihydro-1,2,4-oxadiazol-5-one (Compound 330a)

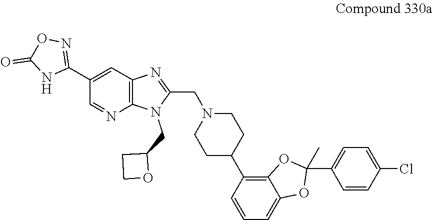 [SMILES: CC1(c2ccc(Cl)cc2)Oc2cccc(C3CCN(Cc4nc5cc(-c6noc(=O)[nH]6)cnc5n4C[C@@H]4CCO4)CC3)c2O1]

3-[2-({4-[2-(4-chlorophenyl)-2-methyl-2H-1,3-benzodioxol-4-yl]piperidin-1-yl}methyl)-3-{[(2S)-oxetan-2-yl]methyl}-3H-imidazo[4,5-b]pyridin-6-yl]-4,5-dihydro-1,2,4-oxadiazol-5-one (Compound 330a) (6.4 mg, yield: 12%) as white solid.

MS Calcd.: 614.2; MS Found: 615.2 [M+H]⁺.

¹H NMR (400 MHz, DMSO-d₆): δ 8.78 (d, J=1.6 Hz, 1H), 8.39 (d, J=1.6 Hz, 1H), 7.59 (d, J=8.4 Hz, 2H), 7.49 (d, J=8.4 Hz, 2H), 6.70-6.80 (m, 3H), 5.15-5.25 (m, 1H), 4.79-4.88 (m, 1H), 4.68-4.76 (m, 1H), 4.44-4.50 (m, 1H), 4.32-4.41 (m, 1H), 3.95-4.05 (m, 2H), 2.92-3.08 (m, 2H), 2.60-2.74 (m, 2H), 2.26-2.51 (m, 3H), 1.97 (s, 3H), 1.70-1.85 (m, 4H).

### Example 182: 1-{[2-({4-[(2S)-2-(4-chloro-2-fluorophenyl)-2-methyl-2H-1,3-benzodioxol-4-yl]piperidin-1-yl}}methyl)-6-[5-(trifluoromethyl)-4H-1,2,4-triazol-3-yl]-3H-imidazo[4,5-c]pyridin-3-yl]methyl}cyclopropane-1-carbonitrile (Compound 345a)

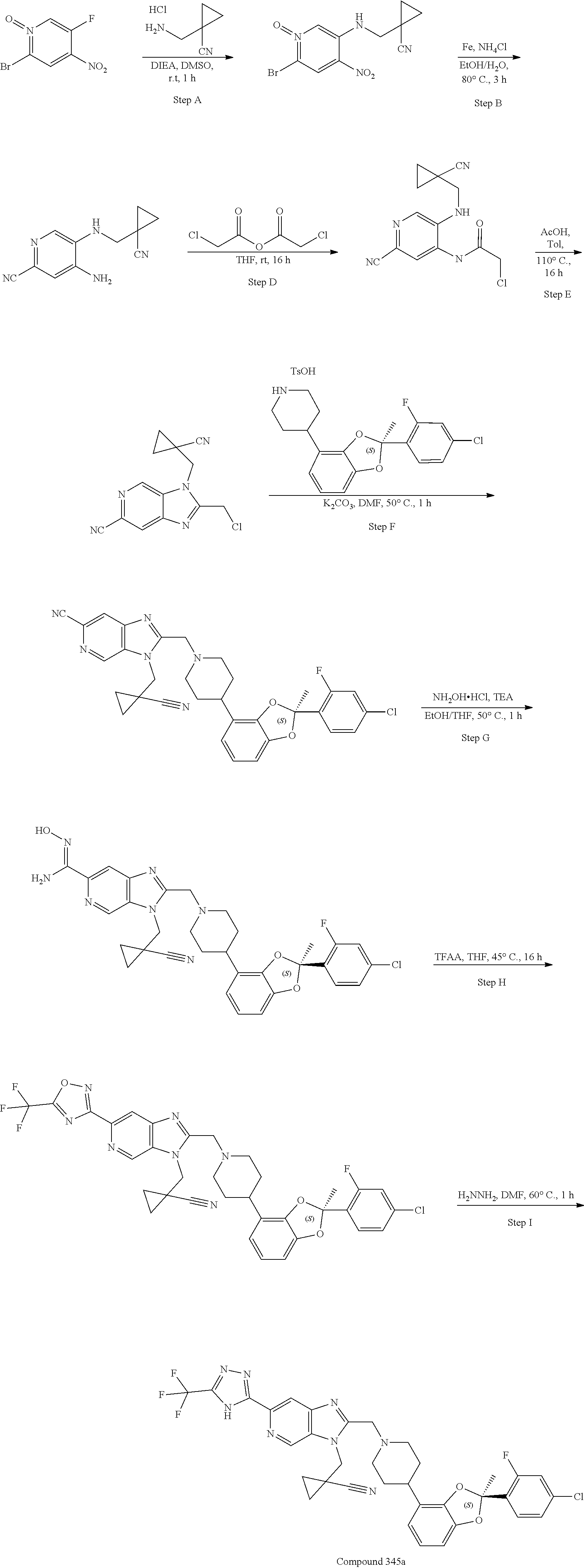

### Step A: 2-bromo-5-(((1-cyanocyclopropyl)methyl)amino)-4-nitropyridine 1-oxide

A mixture of 2-bromo-5-fluoro-4-nitropyridine 1-oxide (1.6 g, 6.78 mmol), 1-(aminomethyl)cyclopropane-1-carbonitrile hydrochloride (890 mg, 6.78 mmol) and DIEA (2.6 g, 20.34 mmol) in DMSO (20 mL) was stirred at rt for 1 hour. After the reaction was completed, the mixture was diluted with EA (50 mL×3), washed with H₂O (20 mL). The organic layer was dried over anhydrous sodium sulfate, filtered and concentrated to dryness. The residue was purified by column chromatography on silica gel (EA/PE=1/1) to give 2-bromo-5-(((1-cyanocyclopropyl)methyl)amino)-4-nitropyridine 1-oxide (2.1 g, yield: 90%) as a yellow solid. MS Calcd.: 312.0; MS Found: 313.0 [M+H]⁺.

### Step B: 1-(((4-amino-6-bromopyridin-3-yl)amino)methyl)cyclopropane-1-carbonitrile

A mixture of 2-bromo-5-(((1-cyanocyclopropyl)methyl)amino)-4-nitropyridine 1-oxide (2.0 g, 6.41 mmol), Fe (1.8 g, 32.05 mmol) and NH₄Cl (3.43 g, 64.10 mmol) in EtOH (20 mL) and H₂O (3 mL) was stirred at 80° C. for 3 hours. After the reaction was completed, the mixture was diluted with DCM (80 mL×3), washed with H₂O (40 mL). The organic layer was dried over anhydrous sodium sulfate, filtered and concentrated in vacuo to dryness. The residue was purified by column chromatography on silica gel (EA/PE=2/1) to give 1-(((4-amino-6-bromopyridin-3-yl)amino)methyl)cyclopropane-1-carbonitrile (1.35 g, yield: 79%) as a yellow solid. MS Calcd.: 266.0; MS Found: 267.0[M+H]⁺.

### Step C: 4-amino-5-(((1-cyanocyclopropyl)methyl)amino)picolinonitrile

A mixture of 1-(((4-amino-6-bromopyridin-3-yl)amino)methyl)cyclopropane-1-carbonitrile (1.2 g, 4.51 mmol), Zn(CN)₂ (1.59 g, 13.53 mmol), Ruphos Pd G₃ (377 mg, 0.45 mmol) and Xphos (430 mg, 0.90 mmol) in NMP (15 mL) was stirred at 130° C. for 10 min under N₂. After the reaction was completed, the mixture was diluted with EA (50 mL×4), washed with H₂O (20 mL). The organic layer was dried over anhydrous sodium sulfate, filtered and concentrated to dryness. The residue was purified by column chromatography on silica gel (EA/PE=1/1) to give 4-amino-5-(((1-cyanocyclopropyl)methyl)amino)picolinonitrile (950 mg, yield: 100%) as yellow oil.

MS Calcd.: 213.1; MS Found: 214.0 [M+H]⁺.

### Step D: 2-chloro-N-(2-cyano-5-(((1-cyanocyclopropyl)methyl)amino)pyridin-4-yl)acetamide

A mixture of 4-amino-5-(((1-cyanocyclopropyl)methyl)amino)picolinonitrile (950 mg, 4.46 mmol) and 2-chloroacetic anhydride (915 mg, 5.35 mmol) in THE (10 ml) was stirred at rt for 16 hours. After the reaction was completed, the mixture was diluted with EA (50 ml×3), washed with H₂O (10 ml). The organic layer was dried over anhydrous sodium sulfate, filtered and concentrated in vacuo to dryness. The residue was purified by column chromatography on silica gel (EA/PE=1/1) to give 2-chloro-N-(2-cyano-5-(((1-cyanocyclopropyl) methyl)amino)pyridin-4-yl)acetamide (1.1 g, yield: 85%). MS Calcd.: 289.1; MS Found: 290.0 [M+H]⁺.

### Step E: 2-(chloromethyl)-3-((1-cyanocyclopropyl)methyl)-3H-imidazo[4,5-c]pyridine-6-carbonitrile

A mixture of 2-chloro-N-(2-cyano-5-(((1-cyanocyclopropyl)methyl)amino)pyridin-4-yl)acetamide (1.1 g, 3.81 mmol) in Toluene (10 mL) was added AcOH (1 mL). The resulting mixture was stirred at 110° C. for 16 hours. After the reaction was completed, the reaction mixture was filtered and the filtrate was concentrated. The crude product was purified by column chromatography on silica gel (EA/PE=2/1) to give 2-(chloromethyl)-3-((1-cyanocyclopropyl)methyl)-3H-imidazo[4,5-c]pyridine-6-carbonitrile (480 mg, yield: 44%) as yellow oil. MS Calcd.: 271.1; MS Found: 290.1 [M+H+18]⁺.

### Step F: (S)-2-((4-(2-(4-chloro-2-fluorophenyl)-2-methylbenzo[d][1,3]dioxol-4-yl)piperidin-1-yl)methyl)-3-((1-cyanocyclopropyl)methyl)-3H-imidazo[4,5-c]pyridine-6-carbonitrile

A mixture of 2-(chloromethyl)-3-((1-cyanocyclopropyl)methyl)-3H-imidazo[4,5-c]pyridine-6-carbonitrile (430 mg, 1.59 mmol) and (S)-4-(2-(4-chloro-2-fluorophenyl)-2-methylbenzo[d][1,3]dioxol-4-yl)piperidine TsOH salt (907.60 mg, 1.75 mmol), K₂CO₃ (658.26 mg, 4.77 mmol) in DMF (10 mL) was stirred at 50° C. for 1 hours. After the reaction was completed, the mixture was diluted with EA (20 mL×3), washed with H₂O (10 mL×3). The organic layer was dried over anhydrous sodium sulfate, filtered and concentrated in vacuo to dryness. The residue was purified by column chromatography on silica gel (EA/PE=1/4) to give (S)-2-((4-(2-(4-chloro-2-fluorophenyl)-2-methylbenzo[d][1,3]dioxol-4-yl)piperidin-1-yl)methyl)-3-((1-cyanocyclopropyl)methyl)-3H-imidazo[4,5-c]pyridine-6-carbonitrile (870 mg, yield: 94%) as brown oil. MS Calcd.: 582.2; MS Found: 583.5 [M+H]⁺.

### Step G: (S)-2-((4-(2-(4-chloro-2-fluorophenyl)-2-methylbenzo[d][1,3]dioxol-4-yl)piperidin-1-yl)methyl)-3-((1-cyanocyclopropyl)methyl)-N′-hydroxy-3H-imidazo[4,5-c]pyridine-6-carboximidamide

A mixture of (S)-2-((4-(2-(4-chloro-2-fluorophenyl)-2-methylbenzo[d][1,3]dioxol-4-yl)piperidin-1-yl)methyl)-3-((1-cyanocyclopropyl)methyl)-3H-imidazo[4,5-c]pyridine-6-carbonitrile (400 mg, 0.68 mmol) and NH₂OH HCl (286 mg, 4.12 mmol), in EtOH/THF (2 mL/2 mL) was added TEA (278 mg, 2.75 mmol) was stirred at 50° C. for 1 hour. After the reaction was completed, the mixture was filtered and the filtrate concentrated to give (S)-2-((4-(2-(4-chloro-2-fluorophenyl)-2-methylbenzo[d][1,3]dioxol-4-yl)piperidin-1-yl)methyl)-3-((1-cyanocyclopropyl)methyl)-N′-hydroxy-3H-imidazo[4,5-c]pyridine-6-carboximidamide (360 mg, crude, yield: 81%) as a white solid. MS Calcd.: 615.2; MS Found: 616.3 [M+H]⁺.

### Step H: (S)-1-((2-((4-(2-(4-chloro-2-fluorophenyl)-2-methylbenzo[d][1,3]dioxol-4-yl)piperidin-1-yl)methyl)-6-(5-(trifluoromethyl)-1,2,4-oxadiazol-3-yl)-3H-imidazo[4,5-c]pyridin-3-yl)methyl) cyclopropane-1-carbonitrile

A mixture of (S)-2-((4-(2-(4-chloro-2-fluorophenyl)-2-methylbenzo[d][1,3]dioxol-4-yl)piperidin-1-yl)methyl)-3-((1-cyanocyclopropyl)methyl)-N′-hydroxy-3H-imidazo[4,5-c]pyridine-6-carboximidamide (200 mg) and TFAA (136 mg, 0.65 mmol) in THE (5 mL) was stirred at 45° C. for 16 hours. After the reaction was completed, the residue was purified by column chromatography on silica gel (EA/PE=1/1) to give (S)-1-((2-((4-(2-(4-chloro-2-fluorophenyl)-2-methylbenzo[d][1,3]dioxol-4-yl)piperidin-1-yl)methyl)-6-(5-(trifluoromethyl)-1,2,4-oxadiazol-3-yl)-3H-imidazo[4,5-c]pyridin-3-yl)methyl)cyclopropane-1-carbonitrile (220 mg, yield: 88%) as a white solid. MS Calcd.: 693.2; MS Found: 694.2 [M+H]⁺.

### Step I: 1-{[2-({4-[(2S)-2-(4-chloro-2-fluorophenyl)-2-methyl-2H-1,3-benzodioxol-4-yl]piperidin-1-yl}methyl)-6-[5-(trifluoromethyl)-4H-1,2,4-triazol-3-yl]-3H-imidazo[4,5-c]pyridin-3-yl]methyl}cyclopropane-1-carbonitrile (Compound 345a)

A mixture of (S)-1-((2-((4-(2-(4-chloro-2-fluorophenyl)-2-methylbenzo[d][1,3]dioxol-4-yl)piperidin-1-yl)methyl)-6-(5-(trifluoromethyl)-1,2,4-oxadiazol-3-yl)-3H-imidazo[4,5-c]pyridin-3-yl)methyl) cyclopropane-1-carbonitrile (100 mg, 0.14 mmol) and N₂H₄·H₂O (9.2 mg, 0.19 mmol) in DMF (1.5 mL) was stirred at 60° C. for 1 hour. After the reaction was completed, the mixture was purified directly by Prep-HPLC (FA) to give 1-{[2-({4-[(2S)-2-(4-chloro-2-fluorophenyl)-2-methyl-2H-1,3-benzodioxol-4-yl]piperidin-1-yl}methyl)-6-[5-(trifluoromethyl)-4H-1,2,4-triazol-3-yl]-3H-imidazo[4,5-c]pyridin-3-yl]methyl}cyclopropane-1-carbonitrile (Compound 345a) (49 mg, yield: 49%).

MS Calcd.: 692.2; MS Found: 693.2 [M+H]⁺.

¹H NMR (400 MHz, DMSO-d₆): δ 9.25 (s, 1H), 8.31 (s, 1H), 7.53-7.60 (m, 2H), 7.33 (dd, J=8.0 Hz, 2.0 Hz, 1H), 6.79-6.80 (m, 2H), 6.71-6.82 (m, 1H), 4.85 (s, 2H), 3.98 (s, 2H), 2.95-3.03 (m, 2H), 2.62-2.71 (m, 1H), 2.22-2.30 (m, 2H), 2.02 (s, 3H), 1.70-1.83 (m, 4H), 1.44-1.52 (m, 4H). ¹⁹F-NMR (377 MHz): −110.76.

### Example 183: 1-{[2-({4-[(2S)-2-(4-chloro-2-fluorophenyl)-2-methyl-2H-1,3-benzodioxol-4-yl]piperidin-1-yl}methyl)-6-(5-oxo-4,5-dihydro-1,2,4-oxadiazol-3-yl)-3H-imidazo[4,5-c]pyridin-3-yl]methyl}cyclopropane-1-carbonitrile (Compound 346a)

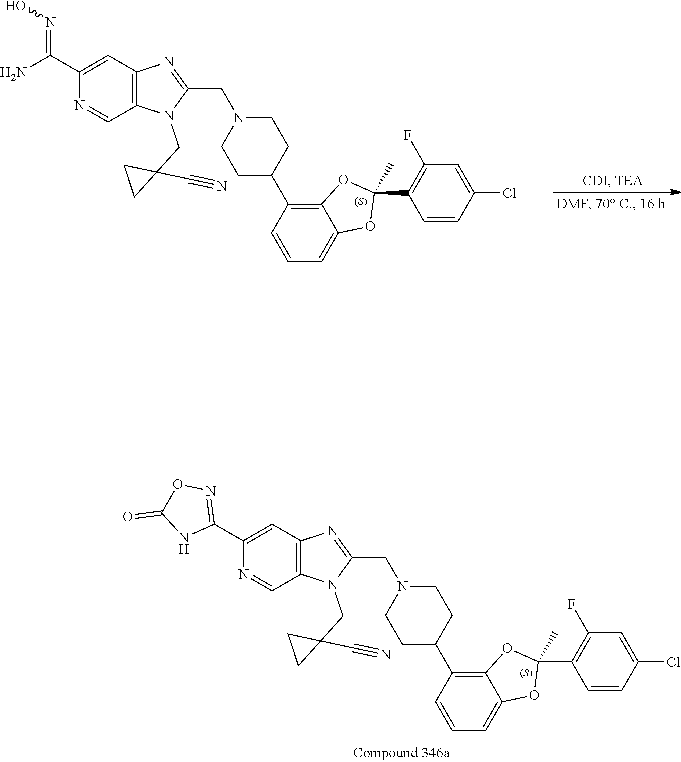

To a solution of (S)-2-((4-(2-(4-chloro-2-fluorophenyl)-2-methylbenzo[d][1,3]dioxol-4-yl)piperidin-1-yl)methyl)-3-((1-cyanocyclopropyl)methyl)-N′-hydroxy-3H-imidazo[4,5-c]pyridine-6-carboximidamide (50 mg) in DMF (1.5 mL) was added TEA (24 mg, 0.24 mmol) and CDI (19 mg, 0.12 mmol). The resulting mixture was stirred at 70° C. for 16 hours. The solvent was removed in vacuo. The residue was purified by Prep-HPLC to give 1-{[2-({4-[(2S)-2-(4-chloro-2-fluorophenyl)-2-methyl-2H-1,3-benzodioxol-4-yl]piperidin-1-yl}methyl)-6-(5-oxo-4,5-dihydro-1,2,4-oxadiazol-3-yl)-3H-imidazo[4,5-c]pyridin-3-yl]methyl}cyclopropane-1-carbonitrile (Compound 346a) (25 mg, yield: 49%) as a white solid.

MS Calcd.: 641.2; MS Found: 642.2 [M+H]⁺.

¹H NMR (400 MHz, DMSO-d₆): δ 9.27 (s, 1H), 8.22 (s, 1H), 7.53-7.60 (m, 2H), 7.32 (dd, J=8.4 Hz, 2.0 Hz, 1H), 6.70-6.80 (m, 3H), 4.85 (s, 2H), 3.99 (s, 2H), 2.95-3.03 (m, 2H), 2.60-2.70 (m, 1H), 2.20-2.30 (m, 2H), 2.02 (s, 3H), 1.70-1.82 (m, 4H), 1.43-1.50 (m, 4H). ¹⁹F-NMR (377 MHz): −110.75.

### Example 184: 3-{2-[(4-{3-[(4-chloro-2-fluorophenyl)methoxy]-4-fluorophenyl}-1,2,3,6-tetrahydropyridin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 299a)

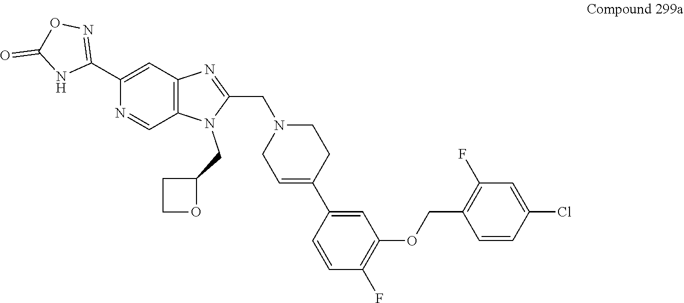 [SMILES: O=c1[nH]c(-c2cc3nc(CN4CC=C(c5ccc(F)c(OCc6ccc(Cl)cc6F)c5)CC4)n(C[C@@H]4CCO4)c3cn2)no1]

Starting from 4-(3-((4-chloro-2-fluorobenzyl)oxy)-4-fluorophenyl)-1,2,3,6-tetrahydropyridine, 3-{2-[(4-{3-[(4-chloro-2-fluorophenyl)methoxy]-4-fluorophenyl}-1,2,3,6-tetrahydropyridin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 299a) (4.7 mg) was obtained as a white solid.

MS Calcd.: 620.2; MS Found: 621.3 [M+H]⁺.

1H NMR (400 MHz, DMSO-d6): δ 9.15 (s, 1H), 7.55-7.65 (m, 1H), 7.50 (d, J=9.6 Hz, 1H), 7.29-7.38 (m, 3H), 7.13-7.22 (m, 1H), 6.98-7.05 (m, 1H), 6.17 (s, 1H), 5.24 (s, 2H), 5.05-5.15 (m, 1H), 4.86-4.95 (m, 1H), 4.70-4.80 (m, 1H), 4.35-4.55 (m, 3H), 4.12 (d, J=13.6 Hz, 1H), 3.99 (d, J=14.4 Hz, 1H), 2.73-2.83 (m, 3H), 2.65-2.73 (m, 2H), 2.39-2.51 (m, 2H). ¹⁹F-NMR (377 MHz): −114.95, −136.47.

### Example 185: 4-{3-[(4-chloro-2-fluorophenyl)methoxy]-4-fluorophenyl}-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-1,2,3,6-tetrahydropyridine (Compound 297a)

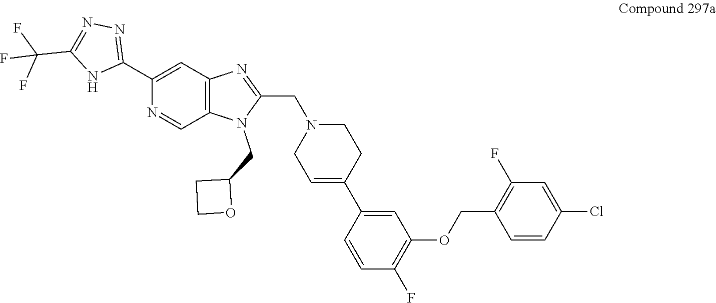 [SMILES: Fc1cc(Cl)ccc1COc1cc(C2=CCN(Cc3nc4cc(-c5nnc(C(F)(F)F)[nH]5)ncc4n3C[C@@H]3CCO3)CC2)ccc1F]

4-{3-[(4-chloro-2-fluorophenyl)methoxy]-4-fluorophenyl}-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-1,2,3,6-tetrahydropyridine (Compound 297a) (19 mg) was obtained as a white solid.

MS Calcd.: 671.2; MS Found: 672.1 [M+H]⁺.

¹H NMR (400 MHz, DMSO-d₆): δ 15.43 (s, 1H), 9.18 (s, 1H), 8.31 (s, 1H), 7.60 (d, J=8.0 Hz, 1H), 7.50 (dd, J=10.0 Hz, 2.0 Hz, 1H), 7.31-7.40 (m, 2H), 7.15-7.23 (m, 1H), 7.01-7.05 (m, 1H), 6.18 (s, 1H), 5.24 (s, 2H), 5.08-5.19 (m, 1H), 4.87-4.97 (m, 1H), 4.72-4.80 (m, 1H), 4.38-4.52 (m, 3H), 4.07-4.20 (m, 1H), 3.95-4.05 (m, 1H), 3.16-3.29 (m, 2H), 2.65-2.85 (m, 3H), 2.30-2.50 (m, 2H). ¹⁹F-NMR (377 MHz): −63.72, −114.96, −136.57.

### Example 186: 4-[(2S)-2-(4-chloro-2-fluorophenyl)-2-methyl-2H-1,3-benzodioxol-4-yl]-1-{[6-(5-cyclopropyl-4H-1,2,4-triazol-3-yl)-3-{[(2S)-oxetan-2-yl]methyl}-3H-imidazo[4,5-c]pyridin-2-yl]methyl}piperidine (Compound 281a)

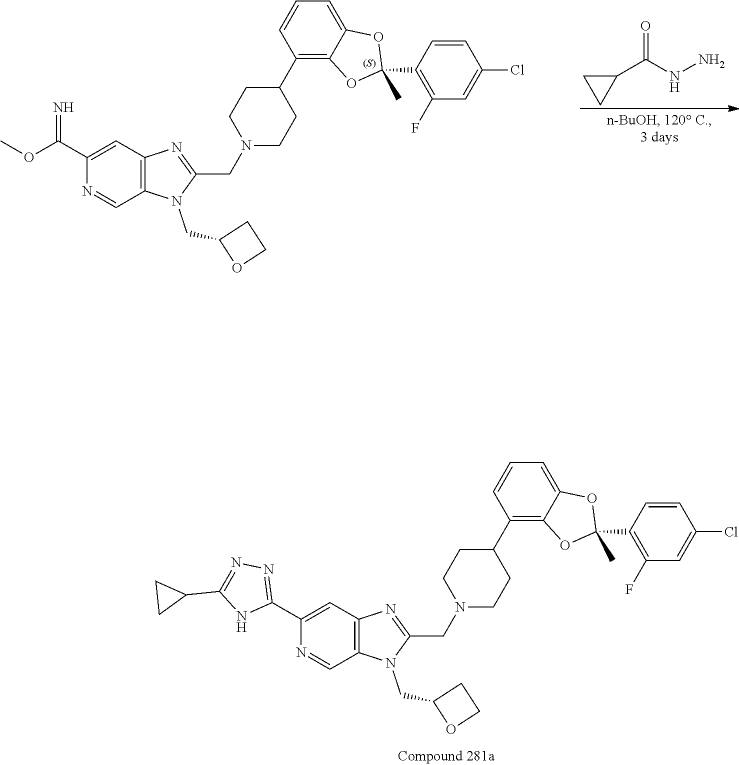

A mixture of methyl 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (23 mg, 0.04 mmol) and cyclopropanecarbohydrazide (7.6 mg, 0.08 mmol), DIEA (15 mg, 0.12 mmol) in n-BuOH (2 mL) was stirred at 120° C. for 3 days. After the reaction was completed, the reaction mixture was concentrated and the residue was purified by Prep-HPLC to give 4-[(2S)-2-(4-chloro-2-fluorophenyl)-2-methyl-2H-1,3-benzodioxol-4-yl]-1-{[6-(5-cyclopropyl-4H-1,2,4-triazol-3-yl)-3-{[(2S)-oxetan-2-yl]methyl}-3H-imidazo[4,5-c]pyridin-2-yl]methyl}piperidine (Compound 281a) (5.4 mg, yield: 21%) as a white solid.

MS Calcd.: 655.2; MS Found: 656.4 [M+H]⁺.

¹H NMR (400 MHz, DMSO-d₆): δ 9.21 (s, 1H), 8.27 (s, 1H), 7.54-7.67 (m, 2H), 7.35 (dd, J=8.8 Hz, 2.0 Hz, 1H), 6.86 (d, J=4.4 Hz, 2H), 6.73-6.80 (m, 1H), 5.05-5.15 (m, 1H), 4.70-4.95 (m, 4H), 4.49-4.56 (m, 1H), 4.37-4.43 (m, 1H), 3.68-3.80 (m, 1H), 3.25-3.40 (m, 1H), 2.90-3.05 (m, 1H), 2.65-2.82 (m, 1H), 2.50-2.61 (m, 1H), 2.33-2.45 (m, 2H), 1.90-2.19 (m, 8H), 0.88-1.02 (m, 4H). ¹⁹F-NMR (377 MHz): −110.51.

### Example 187: 5-chloro-2-[4-(1-{[6-(5-cyclopropyl-4H-1,2,4-triazol-3-yl)-3-{[(2S)-oxetan-2-yl]methyl}-3H-imidazo[4,5-c]pyridin-2-yl]methyl}piperidin-4-yl)-2-methyl-2H-1,3-benzodioxol-2-yl]pyridine (Compound 266a)

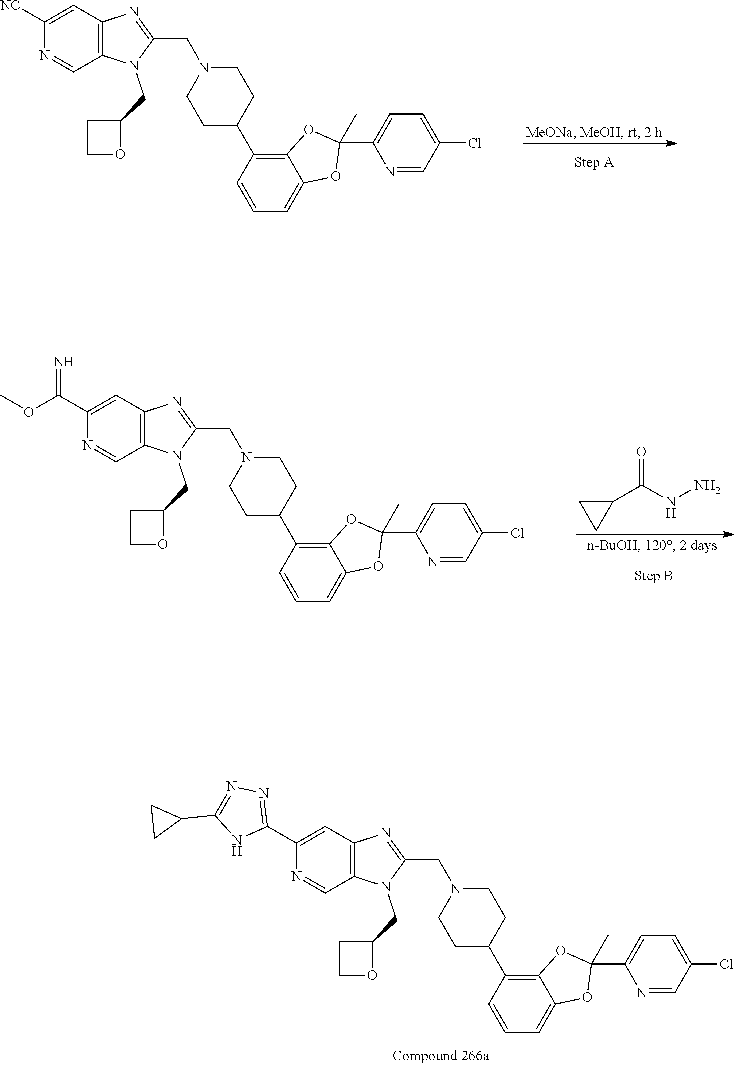 [SMILES: CC1(c2ccc(Cl)cn2)Oc2cccc(C3CCN(Cc4nc5cc(C#N)ncc5n4C[C@@H]4CCO4)CC3)c2O1 ; COC(=N)c1cc2nc(CN3CCC(c4cccc5c4OC(C)(c4ccc(Cl)cn4)O5)CC3)n(C[C@@H]3CCO3)c2cn1 ; NNC(=O)C1CC1 ; CC1(c2ccc(Cl)cn2)Oc2cccc(C3CCN(Cc4nc5cc(-c6nnc(C7CC7)[nH]6)ncc5n4C[C@@H]4CCO4)CC3)c2O1]

### Step A: methyl 2-((4-(2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate

A mixture of 2-((4-(2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (77 mg, 0.14 mmol), MeONa (75 mg, 1.38 mmol) in MeOH (2 mL) was stirred at room temperature for 2 h. After the reaction was completed, the mixture was concentrated in vacuum. The residue was purified by Prep-TLC (MeOH/DCM=1/20) to afford methyl 2-((4-(2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (76 mg, yield: 94%) as a white solid. MS Calcd.: 588.2; MS Found: 589.2[M+H]⁺.

### Step B: 5-chloro-2-[4-(1-{[6-(5-cyclopropyl-4H-1,2,4-triazol-3-yl)-3-{[(2S)-oxetan-2-yl]methyl}-3H-imidazo[4,5-c]pyridin-2-yl]methyl}piperidin-4-yl)-2-methyl-2H-1,3-benzodioxol-2-yl]pyridine (Compound 266a)

A mixture of methyl 2-((4-(2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (76 mg, 0.13 mmol), cyclopropanecarbohydrazide (26 mg, 0.26 mmol), DIEA (50 mg, 0.39 mmol) in n-BuOH (2 mL) was stirred at 120° C. for 2 days. After the reaction was completed. The reaction mixture was concentrated and the residue purified by Prep-HPLC to give 5-chloro-2-[4-(1-{[6-(5-cyclopropyl-4H-1,2,4-triazol-3-yl)-3-f{[(2S)-oxetan-2-yl]methyl}-3H-imidazo[4,5-c]pyridin-2-yl]methyl}piperidin-4-yl)-2-methyl-2H-1,3-benzodioxol-2-yl]pyridine (Compound 266a) (38 mg, yield: 47%) as a white solid.

MS Calcd.: 638.2; MS Found: 639.4 [M+H]⁺.

¹H NMR (400 MHz, DMSO-d₆): δ 9.22 (d, J=0.4 Hz, 1H), 8.74 (dd, J=2.4 Hz, J=0.4 Hz, 1H), 8.28 (d, J=0.8 Hz, 1H), 8.03 (dd, J=8.4 Hz, 2.4 Hz, 1H), 7.65 (dd, J=8.4 Hz, J=0.8 Hz, 1H), 6.85-6.90 (m, 2H), 6.75-6.80 (m, 1H), 5.06-5.14 (m, 1H), 4.80-4.94 (m, 3H), 4.70-4.77 (m, 1H), 4.48-4.54 (m, 1H), 4.35-4.44 (m, 1H), 3.70-3.80 (m, 2H), 3.23-3.40 (m, 2H), 2.90-3.03 (m, 1H), 2.70-2.80 (m, 1H), 2.30-2.43 (m, 1H), 1.95-2.20 (m, 8H), 0.93-1.00 (m, 4H).

### Example 188: 5-chloro-2-[2-methyl-4-(1-{[6-(5-methyl-4H-1,2,4-triazol-3-yl)-3-{[(2S)-oxetan-2-yl]methyl}-3H-imidazo[4,5-c]pyridin-2-yl]methyl}piperidin-4-yl)-2H-1,3-benzodioxol-2-yl]pyridine (Compound 267a)

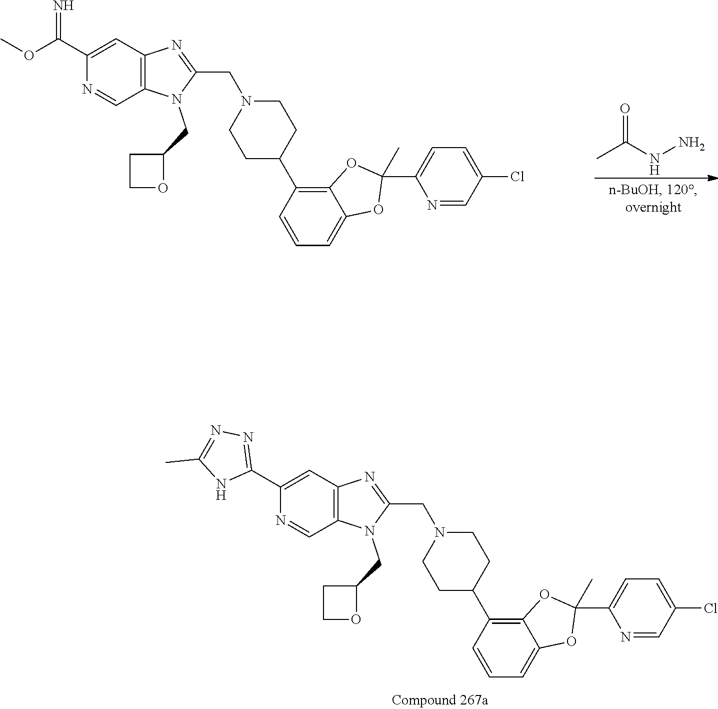

A mixture of methyl 2-((4-(2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (40 mg, 0.07 mmol), acetohydrazide (10 mg, 0.14 mmol), DIEA (45 mg, 0.35 mmol) in n-BuOH (2 mL) was stirred at 120° C. overnight. After the reaction was completed, the reaction mixture was concentrated and purified by Prep-HPLC to give 5-chloro-2-[2-methyl-4-(1-{[6-(5-methyl-4H-1,2,4-triazol-3-yl)-3-{[(2S)-oxetan-2-yl]methyl}-3H-imidazo[4,5-c]pyridin-2-yl]methyl}piperidin-4-yl)-2H-1,3-benzodioxol-2-yl]pyridine (Compound 267a) (22 mg, yield: 52%) as a white solid.

MS Calcd.: 612.2; MS Found: 613.0 [M+H]⁺.

¹H NMR (400 MHz, DMSO-d₆): δ 9.24 (s, 1H), 8.74 (d, J=2.0 Hz, 1H), 8.33 (s, 1H), 8.03 (dd, J=8.4 Hz, 2.4 Hz, 1H), 7.65 (d, J=8.8 Hz, 1H), 6.85-6.90 (m, 2H), 6.75-6.82 (m, 1H), 5.06-5.14 (m, 1H), 4.80-4.95 (m, 3H), 4.72-4.78 (m, 1H), 4.48-4.55 (m, 1H), 4.36-4.44 (m, 1H), 3.70-3.80 (m, 2H), 3.25-3.40 (m, 2H), 2.90-3.05 (m, 1H), 2.70-2.80 (m, 1H), 2.31-2.42 (m, 4H), 1.95-2.20 (m, 7H).

### Example 189: 2-[(4-{2-[(4-chloro-2-fluorophenyl)methoxy]pyridin-3-yl}piperidin-1-yl)methyl]-1-{[(2S)-oxetan-2-yl]methyl}-5-[5-(trifluoromethyl)-4H-1,2,4-triazol-3-yl]-1H-1,3-benzodiazole (Compound 393a)

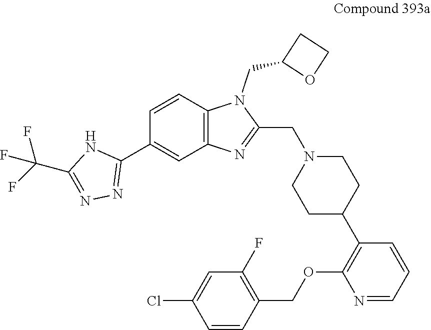 [SMILES: Fc1cc(Cl)ccc1COc1ncccc1C1CCN(Cc2nc3cc(-c4nnc(C(F)(F)F)[nH]4)ccc3n2C[C@@H]2CCO2)CC1]

Starting from 2-((4-chloro-2-fluorobenzyl)oxy)-3-(piperidin-4-yl)pyridine, 2-[(4-{2-[(4-chloro-2-fluorophenyl)methoxy]pyridin-3-yl}piperidin-1-yl)methyl]-1-{[(2S)-oxetan-2-yl]methyl}-5-[5-(trifluoromethyl)-4H-1,2,4-triazol-3-yl]-1H-1,3-benzodiazole (Compound 393a) (40 mg) was obtained as a white solid.

MS Calcd.: 655.2; MS Found: 656.1 [M+H]⁺.

¹H NMR (400 MHz, DMSO-d₆): δ 8.23 (d, J=0.8 Hz, 1H), 8.01 (dd, J=4.8, 1.6 Hz, 1H), 7.93 (dd, J=8.4, 1.6 Hz, 1H), 7.74 (d, J=8.4 Hz, 1H), 7.60 (dd, J=7.6, 1.6 Hz, 1H), 7.55 (t, J=8.0 Hz, 1H), 7.49 (dd, J=10.0, 1.6 Hz, 1H), 7.33 (dd, J=8.0, 1.6 Hz, 1H), 6.98 (dd, J=7.2, 4.8 Hz, 1H), 5.41 (s, 2H), 5.06-5.14 (m, 1H), 4.70-4.79 (m, 1H), 4.56-4.64 (m, 1H), 4.46-4.53 (m, 1H), 4.35-4.42 (m, 1H), 3.92 (d, J=13.6 Hz, 1H), 3.79 (d, J=13.6 Hz, 1H), 2.95-3.03 (m, 1H), 2.85-2.93 (m, 1H), 2.65-2.83 (m, 2H), 2.35-2.48 (m, 1H), 2.14-2.27 (m, 2H), 1.70-1.80 (m, 2H), 1.55-1.70 (m, 2H). ¹⁹F-NMR (377 MHz): −62.81, −115.30.

### Example 190: 2-[(4-chloro-2-fluorophenyl)methoxy]-1′-[(1-{[(2S)-oxetan-2-yl]methyl}-5-[5-(trifluoromethyl)-4H-1,2,4-triazol-3-yl]-1H-1,3-benzodiazol-2-yl)methyl]-1′,2′,3′,6′-tetrahydro-3,4′-bipyridine (Compound 390a)

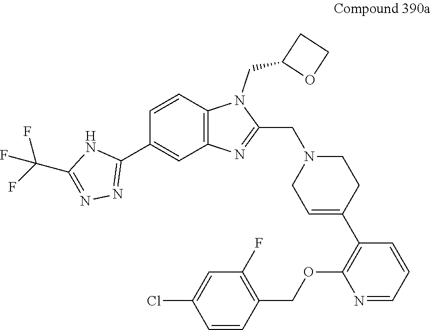 [SMILES: Fc1cc(Cl)ccc1COc1ncccc1C1=CCN(Cc2nc3cc(-c4nnc(C(F)(F)F)[nH]4)ccc3n2C[C@@H]2CCO2)CC1]

Starting from 2-((4-chloro-2-fluorobenzyl)oxy)-1′,2′,3′,6′-tetrahydro-3,4′-bipyridine, 2-[(4-chloro-2-fluorophenyl)methoxy]-1′-[(1-{[(2S)-oxetan-2-yl]methyl}-5-[5-(trifluoromethyl)-4H-1,2,4-triazol-3-yl]-1H-1,3-benzodiazol-2-yl)methyl]-1′,2′,3′,6′-tetrahydro-3,4′-bipyridine (Compound 390a) (18 mg) was obtained as a white solid.

MS Calcd.: 653.2; MS Found: 654.2 [M+H]⁺.

¹H NMR (400 MHz, DMSO-d₆): δ15.16 (brs, 1H), 8.27 (s, 1H), 8.07 (dd, J=5.2, 1.6 Hz, 1H), 7.92 (dd, J=8.8, 1.6 Hz, 1H), 7.81 (d, J=8.4 Hz, 1H), 7.59 (dd, J=7.6, 2.0 Hz, 1H), 7.53 (t, J=8.4 Hz, 1H), 7.48 (dd, J=10.0, 1.6 Hz, 1H), 7.31 (dd, J=8.0, 1.6 Hz, 1H), 7.01 (dd, J=7.2, 5.2 Hz, 1H), 5.92-5.98 (m, 1H), 5.40 (s, 2H), 5.02-5.10 (m, 1H), 4.70-4.78 (m, 1H), 4.57-4.62 (m, 1H), 4.40-4.47 (m, 1H), 4.32-4.40 (m, 1 H), 4.03 (d, J=13.6 Hz, 1H), 3.89 (d, J=13.6 Hz, 1H), 3.10-3.20 (m, 2H), 2.65-2.75 (m, 2H), 2.50-2.63 (m, 1H), 2.40-2.48 (m, 2H), 2.33-2.40 (m, 1H). ¹⁹F-NMR (377 MHz): −63.70, −115.19.

### Example 191: 3-[2-({4-[(2R)-2-(4-chlorophenyl)-2,3-dihydro-1,4-benzodioxin-5-yl]piperidin-1-yl}methyl)-3-{[(2S)-oxetan-2-yl]methyl}-3H-imidazo[4,5-b]pyridin-6-yl]-4,5-dihydro-1,2,4-oxadiazol-5-one (Compound 407a)

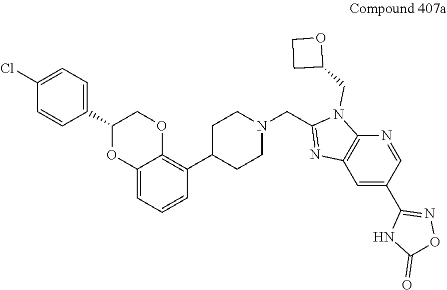 [SMILES: O=c1[nH]c(-c2cnc3c(c2)nc(CN2CCC(c4cccc5c4OC[C@@H](c4ccc(Cl)cc4)O5)CC2)n3C[C@@H]2CCO2)no1]

Starting from (R)-4-(2-(4-chlorophenyl)-2,3-dihydrobenzo[b][1,4]dioxin-5-yl)piperidine and (S)-2-(chloromethyl)-3-(oxetan-2-ylmethyl)-3H-imidazo[4,5-b]pyridine-6-carbonitrile, 3-[2-({4-[(2R)-2-(4-chlorophenyl)-2,3-dihydro-1,4-benzodioxin-5-yl]piperidin-1-yl}methyl)-3-{[(2S)-oxetan-2-yl]methyl}-3H-imidazo[4,5-b]pyridin-6-yl]-4,5-dihydro-1,2,4-oxadiazol-5-one (Compound 407a) (23 mg) was obtained.

MS Calcd.: 614.2; MS Found: 615.4 [M+H]⁺.

¹H NMR (400 MHz, DMSO-d₆): δ 8.77 (d, J=1.6 Hz, 1H), 8.37 (s, 1H), 7.48-7.53 (m, 4H), 6.78-6.84 (m, 3H), 5.22-5.26 (m, 1H), 5.13-5.22 (m, 1H), 4.80-4.88 (m, 1H), 4.69-4.76 (m, 1H), 4.46-4.53 (m, 2H), 4.32-4.38 (m, 1H), 4.02-4.09 (m, 1H), 3.98 (s, 2H), 2.80-3.03 (m, 3H), 2.65-2.72 (m, 1H), 2.40-2.51 (m, 1H), 2.22-2.33 (m, 2H), 1.60-1.80 (m, 4H).

### Example 192: 4-[(2R)-2-(4-chlorophenyl)-2,3-dihydro-1,4-benzodioxin-5-yl]-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidine (Compound 408a)

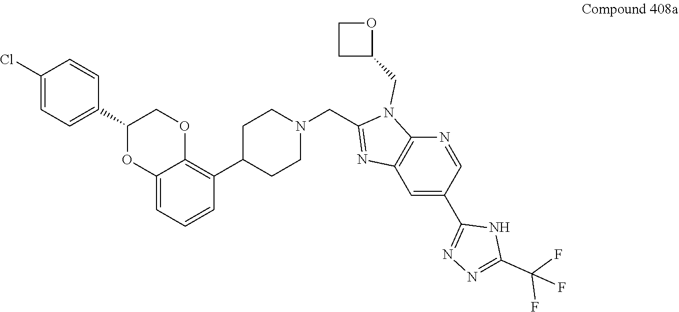 [SMILES: FC(F)(F)c1nnc(-c2cnc3c(c2)nc(CN2CCC(c4cccc5c4OC[C@@H](c4ccc(Cl)cc4)O5)CC2)n3C[C@@H]2CCO2)[nH]1]

4-[(2R)-2-(4-chlorophenyl)-2,3-dihydro-1,4-benzodioxin-5-yl]-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidine (Compound 408a) (10 mg) was obtained.

MS Calcd.: 665.2; MS Found: 666.7 [M+H]⁺.

¹H NMR (400 MHz, DMSO-d₆): δ 9.02 (s, 1H), 8.62 (s, 1H), 7.40-7.53 (m, 4H), 6.73-6.88 (m, 3H), 5.23-5.28 (m, 1H), 5.13-5.22 (m, 1H), 4.80-4.90 (m, 1H), 4.68-4.77 (m, 1H), 4.47-4.53 (m, 2H), 4.31-4.39 (m, 1H), 3.90-4.10 (m, 2H), 2.63-3.10 (m, 4H), 2.38-2.51 (m, 2H), 2.10-2.42 (m, 2H), 1.60-1.89 (m, 4H). ¹⁹F-NMR (377 MHz): −63.73.

### Example 193 5-(3-bromo-1,2,4-thiadiazol-5-yl)-2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazole (Compound 409a)

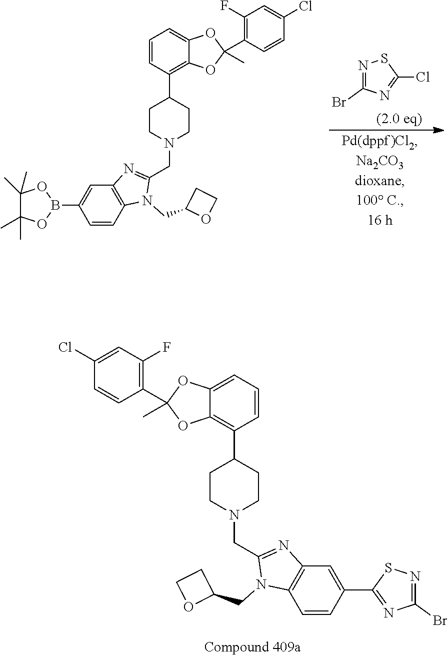 [SMILES: CC1(c2ccc(Cl)cc2F)Oc2cccc(C3CCN(Cc4nc5cc(B6OC(C)(C)C(C)(C)O6)ccc5n4C[C@@H]4CCO4)CC3)c2O1 ; Clc1nc(Br)ns1 ; CC1(c2ccc(Cl)cc2F)Oc2cccc(C3CCN(Cc4nc5cc(-c6nc(Br)ns6)ccc5n4C[C@@H]4CCO4)CC3)c2O1]

A mixture of 2-((4-(2-(4-chloro-2-fluorophenyl)-2-methylbenzo[d][1,3]dioxol-4-yl)piperidin-1-yl)methyl)-1-(((S)-oxetan-2-yl)methyl)-5-(4,4,5,5-tetramethyl-1,3-dioxolan-2-yl)-1H-benzo[d]imidazole (704 mg, 0.11 mmol), Pd(dppf)Cl₂ (73.4 mg, 0.10 mmol), 3-bromo-5-chloro-1,2,4-thiadiazole (417 mg, 2.1 mmol) and Na₂CO₃ (333 mg, 3.1 mmol) in dioxane (10 mL) and H₂O (2 mL) was stirred at 100° C. for 16 hours. After the reaction was completed, the reaction mixture was purified directly by Prep-HPLC to give 5-(3-bromo-1,2,4-thiadiazol-5-yl)-2-({4-[2-(4-chloro-2-fluorophenyl)-2-methyl-2H-1,3-benzodioxol-4-yl]piperidin-1-yl}methyl)-1-{[(2S)-oxetan-2-yl]methyl}-1H-1,3-benzodiazole (Compound 409a) (5.2 mg, 0.7% yield).

MS Calcd.: 709.1; MS Found: 710.1 [M+H]⁺.

¹H NMR (400 MHz, DMSO-d₆): δ 8.29 (d, J=2.0 Hz, 1H), 7.83-7.93 (m, 2H), 7.52-7.60 (m, 2H), 7.34 (dd, J=8.4 Hz, 2.0 Hz, 1H), 6.72-6.80 (m, 3H), 5.10-5.18 (m, 1H), 4.75-4.82 (m, 1H), 4.61-4.69 (m, 1H), 4.38-4.53 (m, 2H), 3.97 (d, J=13.2 Hz, 1H), 3.81 (d, J=13.6 Hz, 1H), 2.98-3.06 (m, 1H), 2.85-2.92 (m, 1H), 2.60-2.76 (m, 2 H), 2.40-2.53 (m, 1H), 2.12-2.30 (m, 2H), 2.03 (s, 3H), 1.68-1.80 (m, 4H). ¹⁹F-NMR (377 MHz): −110.82.

### Example 194A and Example 194B: 3-fluoro-4-[(2S)-2-methyl-4-(1-{[5-(5-methyl-4H-1,2,4-triazol-3-yl)-1-{[(2S)-oxetan-2-yl]methyl}-1H-1,3-benzodiazol-2-yl]methyl}piperidin-4-yl)-2H-1,3-benzodioxol-2-yl]benzonitrile (Compound 410a) and 3-fluoro-4-[(2S)-2-methyl-4-(1-{[5-(5-methyl-4H-1,2,4-triazol-3-yl)-1-{[(2S)-oxetan-2-yl]methyl}-1H-1,3-benzodiazol-2-yl]methyl}piperidin-4-yl)-2H-1,3-benzodioxol-2-yl]benzamide (Compound 410b)

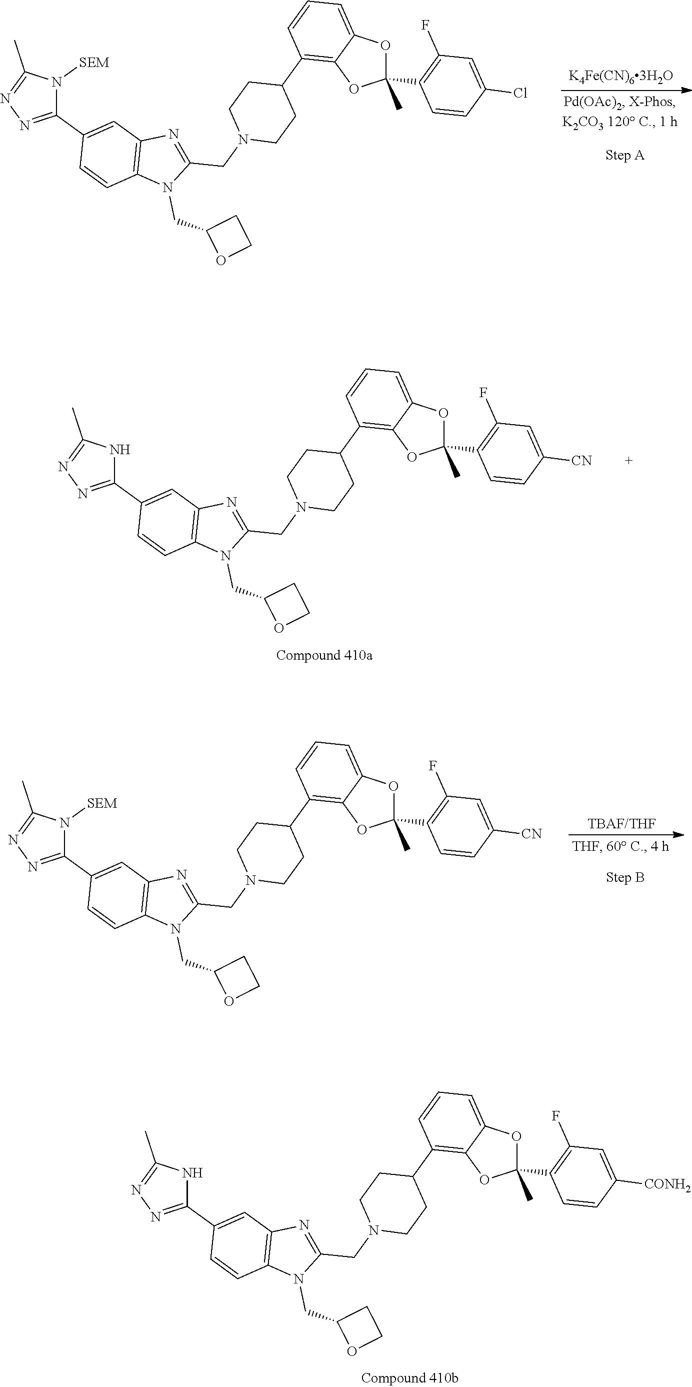 [SMILES: Cc1nnc(-c2ccc3c(c2)nc(CN2CCC(c4cccc5c4O[C@@](C)(c4ccc(Cl)cc4F)O5)CC2)n3C[C@@H]2CCO2)n1COCC[Si](C)(C)C ; Cc1nnc(-c2ccc3c(c2)nc(CN2CCC(c4cccc5c4O[C@@](C)(c4ccc(C#N)cc4F)O5)CC2)n3C[C@@H]2CCO2)[nH]1 ; Cc1nnc(-c2ccc3c(c2)nc(CN2CCC(c4cccc5c4O[C@@](C)(c4ccc(C#N)cc4F)O5)CC2)n3C[C@@H]2CCO2)n1COCC[Si](C)(C)C ; Cc1nnc(-c2ccc3c(c2)nc(CN2CCC(c4cccc5c4O[C@@](C)(c4ccc(C(N)=O)cc4F)O5)CC2)n3C[C@@H]2CCO2)[nH]1]

### Step A: 3-fluoro-4-((S)-2-methyl-4-(1-((5-(5-methyl-4H-1,2,4-triazol-3-yl)-1-(((S)-oxetan-2-yl)methyl)-1H-benzo[d]imidazol-2-yl)methyl)piperidin-4-yl)benzo[d][1,3]dioxol-2-yl)benzonitrile (Compound 410a) & 3-fluoro-4-((S)-2-methyl-4-(1-((5-(5-methyl-4-((2-(trimethylsilyl)ethoxy)methyl)-4H-1,2,4-triazol-3-yl)-1-(((S)-oxetan-2-yl)methyl)-1H-benzo[d]imidazol-2-yl)methyl)piperidin-4-yl)benzo[d][1,3]dioxol-2-yl)benzonitrile

To a solution of 2-((4-((S)-2-(4-chloro-2-fluorophenyl)-2-methylbenzo[d][1,3]dioxol-4-yl) piperidin-1-yl)methyl)-5-(5-methyl-4-((2-(trimethylsilyl)ethoxy)methyl)-4H-1,2,4-triazol-3-yl)-1-((S)-oxetan-2-ylmethyl)-1H-benzo[d]imidazole (50 mg, 0.066 mmol), K₄Fe(CN)₆·3H₂O (28 mg, 0.066 mmol), Pd(OAc)₂ (1.48 mg, 0.0066 mmol), Xphos (6.3 mg, 0.013 mmol) and K₂CO₃ (27 mg, 0.20 mmol) in 1,4-dioxane/H₂O (1 mL/0.2 mL) was stirred at 120° C. with M.W. under Ar for 1 hours. The mixture was filtered and the filtrate was purified by prep-HPLC (0.1% FA) to obtain 3-fluoro-4-[(2S)-2-methyl-4-(1-{[5-(5-methyl-4H-1,2,4-triazol-3-yl)-1-{[(2S)-oxetan-2-yl]methyl}-1H-1,3-benzodiazol-2-yl]methyl}piperidin-4-yl)-2H-1,3-benzodioxol-2-yl]benzonitrile (Compound 410a) (1.0 mg, yield: 2.4%) as a white solid. MS Calcd: 619.3; MS Found: 620.3 [M+H]⁺& also to obtain 3-fluoro-4-((S)-2-methyl-4-(1-((5-(5-methyl-4-((2-(trimethylsilyl)ethoxy)methyl)-4H-1,2,4-triazol-3-yl)-1-(((S)-oxetan-2-yl)methyl)-1H-benzo[d]imidazol-2-yl)methyl)piperidin-4-yl)benzo[d][1,3]dioxol-2-yl)benzonitrile (30 mg, yield: 60%) as a yellow solid. 3-fluoro-4-((S)-2-methyl-4-(1-((5-(5-methyl-4-((2-(trimethylsilyl)ethoxy)methyl)-4H-1,2,4-triazol-3-yl)-1-(((S)-oxetan-2-yl)methyl)-1H-benzo[d]imidazol-2-yl)methyl)piperidin-4-yl)benzo[d][1,3]dioxol-2-yl)benzonitrile:

MS Calcd: 749.4; MS Found: 750.2 [M+H]⁺.

3-fluoro-4-[(2S)-2-methyl-4-(1-{[5-(5-methyl-4H-1,2,4-triazol-3-yl)-1-{[(2S)-oxetan-2-yl]methyl}-1H-1,3-benzodiazol-2-yl]methyl}piperidin-4-yl)-2H-1,3-benzodioxol-2-yl]benzonitrile (HCOOH salt) (Compound 410a)

MS Calcd: 619.3; MS Found: 620.3 [M+H]⁺.

¹H NMR (400 MHz, CD₃OD) δ 8.49 (s, 1H), 8.26 (br.s, 1H), 7.95 (br.s, 1H), 7.82-7.68 (m, 2H), 7.64 (d, J=10.80 Hz, 1H), 7.57 (d, J=8.0 Hz, 1H), 6.82-6.69 (m, 3H), 5.33-5.23 (m, 1H), 4.74-4.60 (m, 1H), 4.57 (br.s, 2H), 4.52-4.40 (m, 1H), 4.07-3.90 (m, 2H), 3.15-3.05 (m, 1H), 3.02-2.95 (m, 1H), 2.90-2.60 (m, 2H), 2.60-2.25 (m, 6H), 2.06 (s, 3H), 2.00-1.75 (m, 4H). ¹⁹F-NMR (377 MHz): −112.08.

### Step B: 3-fluoro-4-((S)-2-methyl-4-(1-((5-(5-methyl-4H-1,2,4-triazol-3-yl)-1-(((S)-oxetan-2-yl)methyl)-1H-benzo[d]imidazol-2-yl)methyl)piperidin-4-yl)benzo[d][1,3]dioxol-2-yl)benzamide (Compound 410b)

To a solution of 3-fluoro-4-((S)-2-methyl-4-(1-((5-(5-methyl-4-((2-(trimethylsilyl)ethoxy)methyl)-4H-1,2,4-triazol-3-yl)-1-(((S)-oxetan-2-yl)methyl)-1H-benzo[d]imidazol-2-yl)methyl)piperidin-4-yl)benzo[d][1,3]dioxol-2-yl)benzonitrile (30 mg, 0.04 mmol) in THE (3 mL) at room temperature was added TBAF/THF (1.0 M, 3 mL). The mixture was heated to 60° C. for stirring 4 hours. The mixture was washed by water, extracted with dichloromethane, the organic phase was washed with brine, dried over Na₂SO₄, the solvents was concentrated under vacuum, the residue was purified by pre-HPLC (0.1% FA) to obtain 3-fluoro-4-((S)-2-methyl-4-(1-((5-(5-methyl-4H-1,2,4-triazol-3-yl)-1-(((S)-oxetan-2-yl)methyl)-1H-benzo[d]imidazol-2-yl)methyl)piperidin-4-yl)benzo[d][1,3]dioxol-2-yl)benzamide (Compound 410b) (2.3 mg, yield: 9.1%, HCOOH salt) as a yellow solid.

MS Calcd: 637.3; MS Found: 638.2 [M+H]⁺.

¹H NMR (400 MHz, CD₃OD) δ 8.45-8.35 (m, 1H), 8.16 (s, 1H), 8.05 (s, 1H), 7.88 (d, J=9.20, 1H), 7.75-7.60 (m, 4H), 7.55 (s, 1H), 6.81-6.72 (m, 3H), 5.17-5.08 (m, 1H), 4.79-4.70 (m, 1H), 4.65-4.57 (m, 1H), 4.51-4.38 (m, 2H), 3.96-3.73 (m, 2H), 3.20-3.13 (m, 1H), 3.05-2.99 (m, 1H), 2.91-2.84 (m, 1H), 2.75-2.60 (m, 2H), 2.37 (s, 3H), 2.30-2.11 (m, 2H), 2.05 (s, 3H), 1.82-1.50 (m, 4H). ¹⁹F-NMR (377 MHz): −113.31.

### Example 195: 2-({4-[(2S)-2-(4-chloro-2-fluorophenyl)-2-methyl-2H-1,3-benzodioxol-4-yl]piperidin-1-yl}methyl)-5-(5-cyclopropyl-4H-1,2,4-triazol-3-yl)-1-{[(2S)-oxetan-2-yl]methyl}-1H-1,3-benzodiazole (Compound 283a)

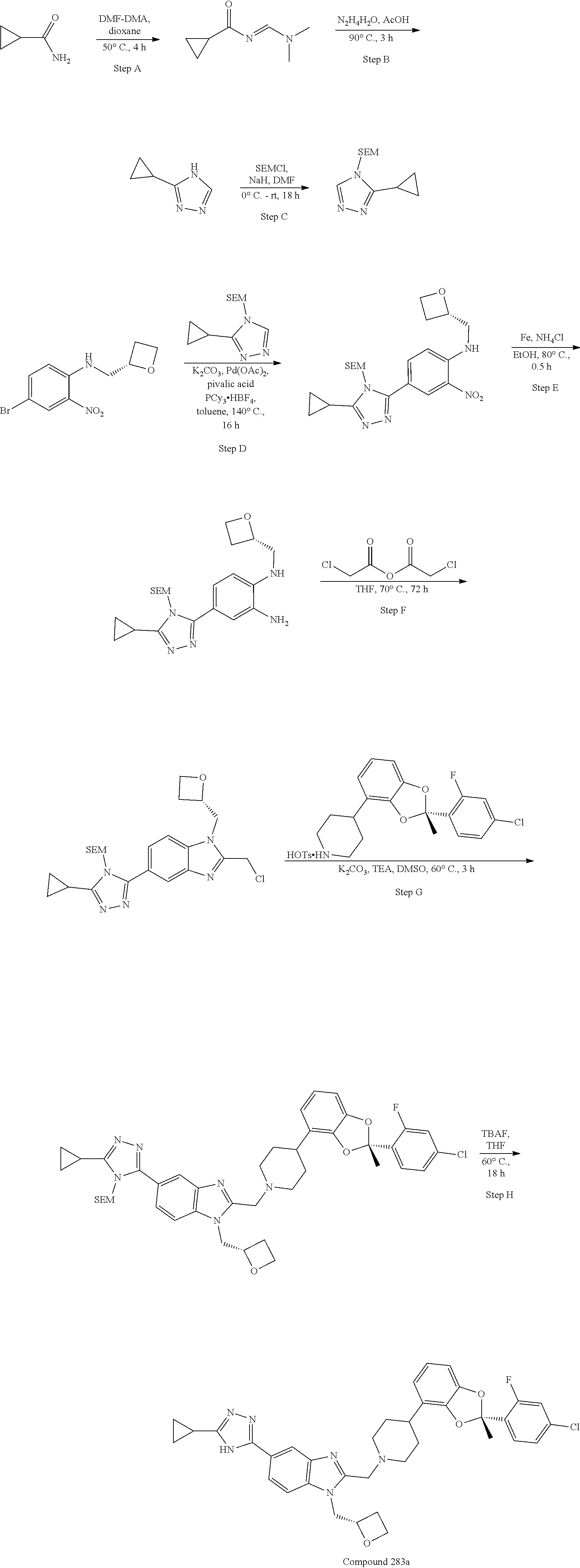

### Step A: (E)-N-((dimethylamino)methylene)cyclopropanecarboxamide

A mixture of DMF-DMA (4.9 mL, 14.0 mmol) and cyclopropanecarboxamide (1.0 g, 12.0 mmol) in dioxane (15 mL) was stirred at 50° C. for 4 hours. Upon cooling down, the mixture was concentrated under vacuum to furnish (E)-N-((dimethylamino)methylene)cyclopropanecarboxamide (1.6 g, crude) as crude colorless oil. MS Calcd.: 140.1; MS Found: 141.1 [M+H]⁺.

### Step B: 3-cyclopropyl-4H-1,2,4-triazole

A mixture of (E)-N-((dimethylamino)methylene)cyclopropanecarboxamide (1.3 g, crude) and N₂H₄H₂O (780 mg, 12.5 mmol) in AcOH (5.0 ml) was stirred at 90° C. for 3 hours. Upon cooling down, the reaction mixture was directly purified by column chromatography (0.1% TFA) to furnish 3-cyclopropyl-4H-1,2,4-triazole (930 mg, crude) as crude yellow oil. MS Calcd.: 109.1; MS Found: 110.4 [M+H]⁺.

¹H NMR (400 MHz, DMSO-d₆): δ 9.47 (brs, 1H), 8.45 (s, 1H), 2.18-2.05 (m, 1H), 1.13-1.01 (m, 2H), 1.00-0.88 (m, 2H).

### Step C: 3-cyclopropyl-4-((2-(trimethylsilyl)ethoxy)methyl)-4H-1,2,4-triazole

To a stirred solution of 3-cyclopropyl-4H-1,2,4-triazole (10.2 g, 93.7 mmol, crude) in dry DMF (120 mL) was added NaH (7.5 g, 187.4 mmol, 60% w/w) in portions at 0° C. The mixture was stirred at 0° C. for 2 hours. To the above mixture was added a solution of SEM-Cl (20.4 g, 121.8 mmol) in DMF (30 ml) dropwise at room temperature. The reaction mixture was stirred at room temperature for 18 hours. The reaction mixture was quenched by the addition of saturated NH₄Cl (300 mL) and extracted with EA (3×80 mL). The combined organic layers were washed with brine (50 mL), dried over anhydrous Na₂SO₄ and concentrated under vacuum. The residue was purified by column chromatography to furnish 3-cyclopropyl-4-((2-(trimethylsilyl)ethoxy)methyl)-4H-1,2,4-triazole (5.7 g, yield: 50%) as colorless oil. MS Calcd.: 239.2; MS Found: 240.2 [M+H]⁺.

The product might be a mixture of the following three structures. We choose b as the representative for subsequent reaction:

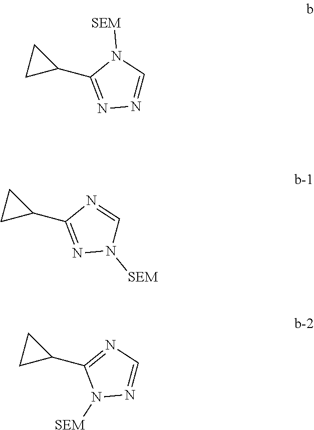 [SMILES: C[Si](C)(C)CCOCn1cnnc1C1CC1 ; C[Si](C)(C)CCOCn1cnc(C2CC2)n1 ; C[Si](C)(C)CCOCn1ncnc1C1CC1]

### Step D: (S)-4-(5-cyclopropyl-4-((2-(trimethylsilyl)ethoxy)methyl)-4H-1,2,4-triazol-3-yl)-2-nitro-N-(oxetan-2-ylmethyl)aniline

A mixture of 3-cyclopropyl-4-((2-(trimethylsilyl)ethoxy)methyl)-4H-1,2,4-triazole (1.7 g, 7.1 mmol), Pd(OAc)₂ (79 mg, 0.35 mmol), PCy₃·HBF₄ (260 mg, 0.706 mmol), pivalic acid (238 mg, 2.3 mmol), K₂CO₃ (2.9 g, 21.2 mmol) and (S)-4-bromo-2-nitro-N-(oxetan-2-ylmethyl)aniline (1.0 g, 3.5 mmol) in toluene (12 mL) was stirred at 140° C. for 16 hours under Ar atmosphere in a sealed tube. Upon cooling down, the mixture was concentrated under vacuum. The residue was purified by a silica gel column to furnish (S)-4-(5-cyclopropyl-4-((2-(trimethylsilyl)ethoxy)methyl)-4H-1,2,4-triazol-3-yl)-2-nitro-N-(oxetan-2-ylmethyl)aniline (210 mg, yield: 13%) as a green oil. MS Calcd.: 445.2; MS Found: 446.2 [M+H]⁺.

### Step E: (S)-4-(5-cyclopropyl-4-((2-(trimethylsilyl)ethoxy)methyl)-4H-1,2,4-triazol-3-yl)-N-(oxetan-2-ylmethyl)benzene-1,2-diamine

A mixture of Fe (604 mg, 9.0 mmol), saturated NH₄Cl (2.0 mL) and (S)-4-(5-cyclopropyl-4-((2-(trimethylsilyl)ethoxy)methyl)-4H-1,2,4-triazol-3-yl)-2-nitro-N-(oxetan-2-ylmethyl)aniline (200 mg, 0.45 mmol) in EtOH (6.0 ml) was stirred at 80° C. for 0.5 hour. The mixture was filtrated and the solid was washed with hot EtOH (5×1.0 mL). The filtrate was concentrated under vacuum to furnish (S)-4-(5-cyclopropyl-4-((2-(trimethylsilyl)ethoxy)methyl)-4H-1,2,4-triazol-3-yl)-N-(oxetan-2-ylmethyl)benzene-1,2-diamine (70 mg, yield: 37%) as an orange solid. MS Calcd.: 415.2; MS Found: 416.2 [M+H]⁺.

### Step F: (S)-2-(chloromethyl)-5-(5-cyclopropyl-4-((2-(trimethylsilyl)ethoxy)methyl)-4H-1,2,4-triazol-3-yl)-1-(oxetan-2-ylmethyl)-1H-benzo[d]imidazole

To a stirred solution of (S)-4-(5-cyclopropyl-4-((2-(trimethylsilyl)ethoxy)methyl)-4H-1,2,4-triazol-3-yl)-N¹-(oxetan-2-ylmethyl)benzene-1,2-diamine (70 mg, 0.17 mmol) in dry THE (8.0 mL) was added a solution of 2-chloroacetic anhydride (20 mg, 0.12 mmol) in THE (1.0 mL) dropwise at 0° C. The mixture was stirred at 0° C. for 10 minutes and at room temperature for 10 minutes and then at 70° C. for 1 day. An additional portion of 2-chloroacetic anhydride (20 mg, 0.118 mmol) in THE (1.0 mL) was added dropwise at room temperature. The final reaction mixture was stirred at 70° C. for another 2 days. Upon cooling down, the mixture was diluted with saturated NaHCO₃ (50 mL) and extracted with EA (15 mL×3). The combined organic layers were dried over anhydrous Na₂SO₄ and concentrated under vacuum. The residue was purified by Prep-TLC to furnish (S)-2-(chloromethyl)-5-(5-cyclopropyl-4-((2-(trimethylsilyl)ethoxy)methyl)-4H-1,2,4-triazol-3-yl)-1-(oxetan-2-ylmethyl)-1H-benzo[d]imidazole (20 mg, yield: 25%) as a white solid. MS Calcd.: 473.2; MS Found: 474.2 [M+H]⁺.

### Step G: 2-((4-((S)-2-(4-chloro-2-fluorophenyl)-2-methylbenzo[d][1,3]dioxol-4-yl)piperidin-1-yl)methyl)-5-(5-cyclopropyl-4-((2-(trimethylsilyl)ethoxy)methyl)-4H-1,2,4-triazol-3-yl)-1-(((S)-oxetan-2-yl)methyl)-1H-benzo[d]imidazol

A mixture of (S)-4-(2-(4-chloro-2-fluorophenyl)-2-methylbenzo[d][1,3]dioxol-4-yl)piperidine 4-methylbenzenesulfonate (22 mg, 0.042 mmol), K₂CO₃ (18 mg, 0.127 mmol) and (S)-2-(chloromethyl)-5-(5-cyclopropyl-4-((2-(trimethylsilyl)ethoxy)methyl)-4H-1,2,4-triazol-3-yl)-1-(oxetan-2-ylmethyl)-1H-benzo[d]imidazole (20 mg, 0.042 mmol) in DMSO (2.0 ml) and TEA (1.0 mL) was at 60° C. for 3 hours. Upon cooling down, the mixture was diluted with H₂O (50 mL) and extracted with EA (15 mL×3). The combined organic layers were washed with brine (35 mL), dried over anhydrous Na₂SO₄ and concentrated under vacuum to furnish 2-((4-((S)-2-(4-chloro-2-fluorophenyl)-2-methylbenzo[d][1,3]dioxol-4-yl)piperidin-1-yl)methyl)-5-(5-cyclopropyl-4-((2-(trimethylsilyl)ethoxy)methyl)-4H-1,2,4-triazol-3-yl)-1-(((S)-oxetan-2-yl)methyl)-1H-benzo[d]imidazole (40 mg, crude) as crude yellow oil. MS Calcd.: 784.3; MS Found: 785.3 [M+H]⁺.

### Step H: 2-({4-[(2S)-2-(4-chloro-2-fluorophenyl)-2-methyl-2H-1,3-benzodioxol-4-yl]piperidin-1-yl}methyl)-5-(5-cyclopropyl-4H-1,2,4-triazol-3-yl)-1-{[(2S)-oxetan-2-yl]methyl}-1H-1,3-benzodiazole (Compound 283a)

A mixture of TBAF (0.2 mL, 0.20 mmol, 1 N in THF) and 2-((4-((S)-2-(4-chloro-2-fluorophenyl)-2-methylbenzo[d][1,3]dioxol-4-yl)piperidin-1-yl)methyl)-5-(5-cyclopropyl-4-((2-(trimethylsilyl)ethoxy)methyl)-4H-1,2,4-triazol-3-yl)-1-(((S)-oxetan-2-yl)methyl)-1H-benzo[d]imidazole (40 mg, crude) in THE (2.0 mL) was stirred at 60° C. for 18 hours. Upon cooling down, the mixture was concentrated under vacuum. The residue was purified by Prep-HPLC (0.1% FA) to furnish 2-({4-[(2S)-2-(4-chloro-2-fluorophenyl)-2-methyl-2H-1,3-benzodioxol-4-yl]piperidin-1-yl}methyl)-5-(5-cyclopropyl-4H-1,2,4-triazol-3-yl)-1-{[(2S)-oxetan-2-yl]methyl}-1H-1,3-benzodiazole (Compound 283a, HCOOH salt) (2.2 mg) as a white solid.

MS Calcd.: 654.2; MS Found: 655.2 [M+H]⁺.

¹H NMR (400 MHz, MeOD): δ 8.48 (br.s, 1H), 8.26 (br.s, 1H), 7.98-7.86 (m, 1H), 7.79-7.64 (m, 1H), 7.58 (t, J=8.0, 1H), 7.27 (dd, J=10.8, 1.6 Hz, 1H), 7.20 (dd, J=8.4, 1.6 Hz, 1H), 6.80-6.68 (m, 3H), 5.32-5.25 (m, 1H), 4.96-4.85 (m, 1H), 4.74-4.57 (m, 1H), 4.50-4.43 (m, 1H), 4.02 (d, J=14.0 Hz, 2H), 3.91 (d, J=14.0 Hz, 1H), 3.15-3.04 (m, 1H), 2.99-2.93 (m, 1H), 2.85-2.64 (m, 2H), 2.59-2.49 (m, 1H), 2.40-2.23 (m, 2H), 2.16-1.74 (m, 8H), 1.19-0.93 (m, 4H). ¹⁹F-NMR (377 MHz): −112.34.

### Example 196: 1-{[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]-1H-1,3-benzodiazol-1-yl]methyl}cyclopropane-1-carbonitrile (Compound 412a)

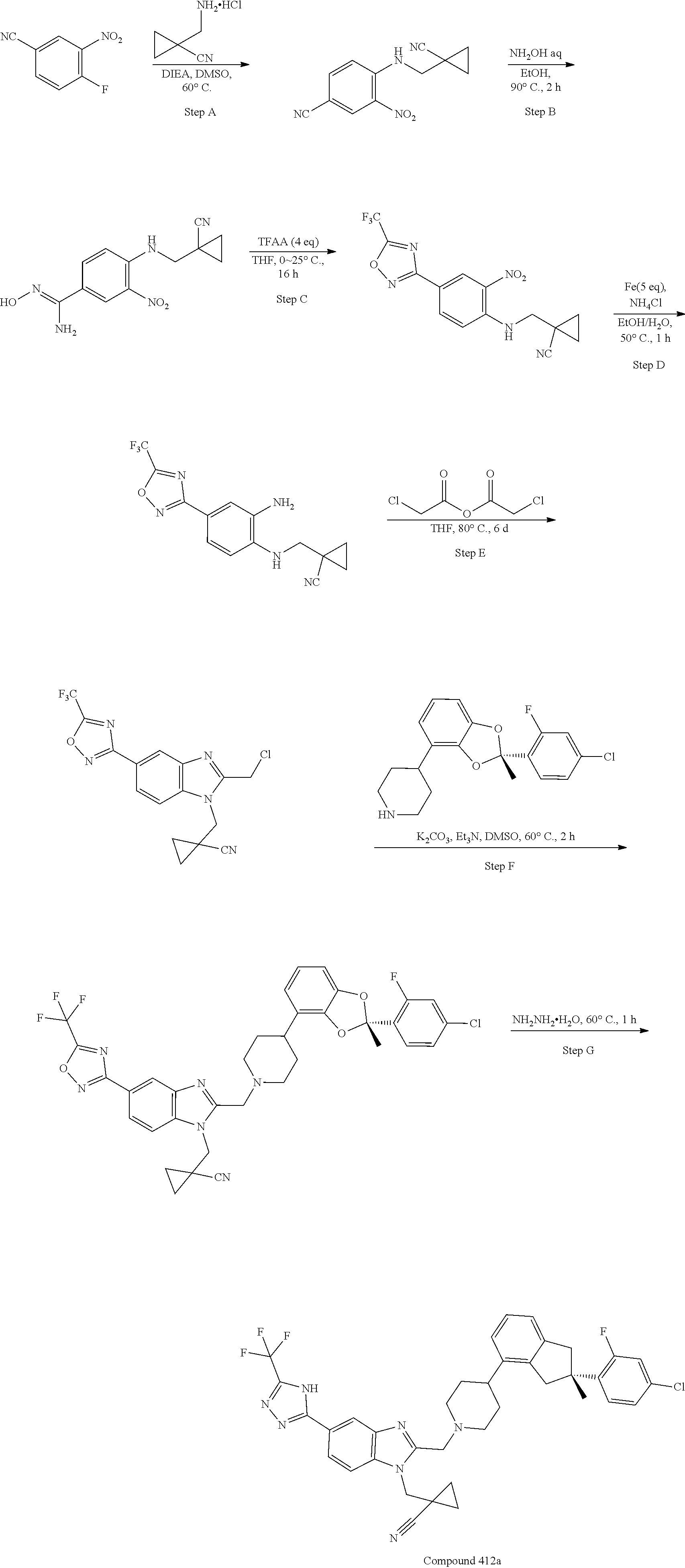

### Step A: 4-(((1-cyanocyclopropyl)methyl)amino)-3-nitrobenzonitrile

A mixture of 4-fluoro-3-nitrobenzonitrile (251 mg, 1.0 mmol) and 1-(aminomethyl)cyclopropane-1-carbonitrile hydrochloride (200 mg, 1.5 mmol), DIEA (2.0 g, 15.2 mmol) in DMSO (30 mL) was stirred at 60° C. for 2 hours. The mixture was poured into water (50 mL) and extracted with EA (3×50 mL), the combined organic layers were washed with brine, dried over sodium sulfate, filtered, and concentrated under reduced pressure. The residue was purified by silica gel chromatography (PE:EA=1:1) to furnish 4-(((1-cyanocyclopropyl)methyl)amino)-3-nitrobenzonitrile (350 mg, yield: 96%) as yellow solid. MS Calcd.: 242.1; MS Found: 241.1 [M+H]⁻.

### Step B: 4-(((1-cyanocyclopropyl)methyl)amino)-N′-hydroxy-3-nitrobenzimidamide

A mixture of 4-(((1-cyanocyclopropyl)methyl)amino)-3-nitrobenzonitrile (300 mg, 1.24 mmol) and NH₂OH·aq (410 mg, 6.21 mmol, 50% wt. in water) in EtOH (50 mL) was stirred at 90° C. for 2 hours. The mixture was poured into cold water (50 mL) and extracted with EA (3×50 mL), the combined organic layers were washed with brine, dried over sodium sulfate, filtered and concentrated under reduced pressure. The residue was purified by column chromatography (DCM:MeOH=10:1) to furnish 4-(((1-cyanocyclopropyl)methyl)amino)-N′-hydroxy-3-nitrobenzimidamide (180 mg, yield: 53%) as yellow solid. MS Calcd.: 275.1; MS Found: 276.1 [M+H]⁺.

### Step C: 1-(((2-nitro-4-(5-(trifluoromethyl)-1,2,4-oxadiazol-3-yl)phenyl)amino) methyl)cyclopropane-1-carbonitrile

A mixture of 4-(((1-cyanocyclopropyl)methyl)amino)-N′-hydroxy-3-nitrobenzimidamide (180 mg, 0.66 mmol) and TFAA (687 mg, 3.3 mmol) in THE (50 mL) was stirred at 0° C. for 10 min, then stirred at 25° C. for 16 hours. The mixture was poured into NaHCO₃.aq (50 mL) and extracted with EA (3×50 mL), the combined organic layers were washed with brine, dried over sodium sulfate, filtered, and concentrated under reduced pressure. The residue was purification by column chromatography (PE:EA=1:1) to give 1-(((2-nitro-4-(5-(trifluoromethyl)-1,2,4-oxadiazol-3-yl)phenyl)amino)methyl)cyclopropane-1-carbonitrile (180 mg, yield: 78%) as a yellow solid. MS Calcd.: 353.1; MS Found: 354.1 [M+H]⁺.

### Step D: 1-(((2-amino-4-(5-(trifluoromethyl)-1,2,4-oxadiazol-3-yl)phenyl)amino) methyl)cyclopropane-1-carbonitrile

A mixture of 1-(((2-nitro-4-(5-(trifluoromethyl)-1,2,4-oxadiazol-3-yl)phenyl)amino)methyl)cyclopropane-1-carbonitrile (150 mg, 0.42 mmol), Fe (119 mg, 2.1 mmol) and NH₄Cl (227 mg, 4.2 mmol) in EtOH (50 mL) and H₂O (25 mL) was stirred at 50° C. for 1 hour. The mixture was filtration and concentrated, the residue was dissolve by water (50 mL) and extracted with EA (3×50 mL), the combined organic layers were washed with brine, dried over sodium sulfate, filtered and concentrated under reduced pressure to give 1-(((2-amino-4-(5-(trifluoromethyl)-1,2,4-oxadiazol-3-yl)phenyl)amino)methyl)cyclopropane-1-carbonitrile (60 mg, yield: 44%) as a yellow solid. MS Calcd.: 323.1; MS Found: 324.1 [M+H]⁺. Step E: 1-((2-(chloromethyl)-5-(5-(trifluoromethyl)-1,2,4-oxadiazol-3-yl)-1H-benzo[d]imidazol-1-yl)methyl)cyclopropane-1-carbonitrile

A mixture of 1-(((2-amino-4-(5-(trifluoromethyl)-1,2,4-oxadiazol-3-yl)phenyl)amino)methyl)cyclopropane-1-carbonitrile (60 mg, 0.19 mmol) and 2-chloroacetic anhydride (32 mg, 0.19 mmol) in THE (50 mL) was stirred at 80° C. for 24 hours. Another portion of 2-chloroacetic anhydride (32 mg, 0.19 mmol) was added and the mixture was stirred at 80° C. for 5 days. The mixture was concentrated and purified by pre-TLC (PE:EA=1:1) to give 1-((2-(chloromethyl)-5-(5-(trifluoro methyl)-1,2,4-oxadiazol-3-yl)-1H-benzo[d]imidazol-1-yl)methyl)cyclopropane-1-carbonitrile (20 mg, yield: 27%) as white solid. MS Calcd.: 381.1; MS Found: 382.2 [M+H]⁺.

### Step F: (S)-1-((2-((4-(2-(4-chloro-2-fluorophenyl)-2-methylbenzo[d][1,3]dioxol-4-yl)piperidin-1-yl)methyl)-5-(5-(trifluoromethyl)-1,2,4-oxadiazol-3-yl)-1H-benzo[d]imidazol-1-yl)methyl)cyclopropane-1-carbonitrile

A mixture of (S)-4-(2-(4-chloro-2-fluorophenyl)-2-methylbenzo[d][1,3]dioxol-4-yl)piperidine (27 mg), K₂CO₃ (22 mg, 0.16 mmol) and Et₃N (27 mg, 0.27 mmol) in DMSO (6 mL) was stirred at 25° C. for 0.5 hour. Then 1-((2-(chloromethyl)-5-(5-(trifluoromethyl)-1,2,4-oxadiazol-3-yl)-1H-benzo[d]imidazol-1-yl)methyl) cyclopropane-1-carbonitrile (20 mg, 0.05 mmol) was added to the mixture was stirred at 60° C. for 2 hours. The mixture was poured into water (30 mL) and extracted with EA (3×30 mL), the combined organic layers were washed with brine, dried over sodium sulfate, filtered, and concentrated under reduced pressure. The residue was purified by pre-TLC (PE:EA=1:1) to give (S)-1-((2-((4-(2-(4-chloro-2-fluorophenyl)-2-methylbenzo[d][1,3]dioxol-4-yl)piperidin-1-yl)methyl)-5-(5-(trifluoromethyl)-1,2,4-oxadiazol-3-yl)-1H-benzo[d]imidazol-1-yl)methyl)cyclopropane-1-carbonitrile (20 mg, yield: 58%) as a colorless oil. MS Calcd.: 692.2; MS Found: 693.4 [M+H]⁺.

### Step G: 1-{[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]-1H-1,3-benzodiazol-1-yl]methyl}cyclopropane-1-carbonitrile (Compound 412a)

A mixture of (S)-1-((2-((4-(2-(4-chloro-2-fluorophenyl)-2-methylbenzo[d][1,3]dioxol-4-yl)piperidin-1-yl)methyl)-5-(5-(trifluoromethyl)-1,2,4-oxadiazol-3-yl)-1H-benzo[d]imidazol-1-yl)methyl)cyclopropane-1-carbonitrile (20 mg, 0.03 mmol) and NH₂NH₂·H₂O (0.5 mL) in EtOH (2 mL) was stirred at 60° C. for 1 hour under N₂. The reaction mixture was purified by prep-HPLC to give 1-{[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]-1H-1,3-benzodiazol-1-yl]methyl}cyclopropane-1-carbonitrile Compound 412a (2.3 mg, yield: 6.6%) as white solid.

MS Calcd.: 691.2; MS Found: 692.2 [M+H]⁺.

¹H NMR (400 MHz, MeOD): δ 8.33 (s, 1H), 8.04 (d, J=8.8 Hz, 1H), 7.85 (d, J=8.4 Hz, 1H), 7.58 (t, J=8.0 Hz, 1H), 7.28 (dd, J=10.8 Hz, 2.0 Hz, 1H), 7.20 (dd, J=8.4 Hz, 1.6 Hz, 1H), 6.80-6.68 (m, 3H), 4.85-4.78 (m, 1H), 4.57 (br.s, 1H), 4.02 (s, 2H), 3.08-3.00 (m, 2H), 2.75-2.65 (m, 1H), 2.38-2.28 (m, 2H), 2.00 (s, 3H), 2.10-1.88 (m, 2H), 1.87-1.78 (m, 2H), 1.49-1.45 (m, 4H). ¹⁹F-NMR (377 MHz): −66.41, −112.33.

### Example 197: 1-{[2-({4-[2-(4-chlorophenyl)-2-methyl-2H-1,3-benzodioxol-4-yl]-1,2,3,6-tetrahydropyridin-1-yl}}methyl)-5-[5-(trifluoromethyl)-4H-1,2,4-triazol-3-yl]-1H-1,3-benzodiazol-1-yl]methyl}cyclopropane-1-carbonitrile (Compound 413)

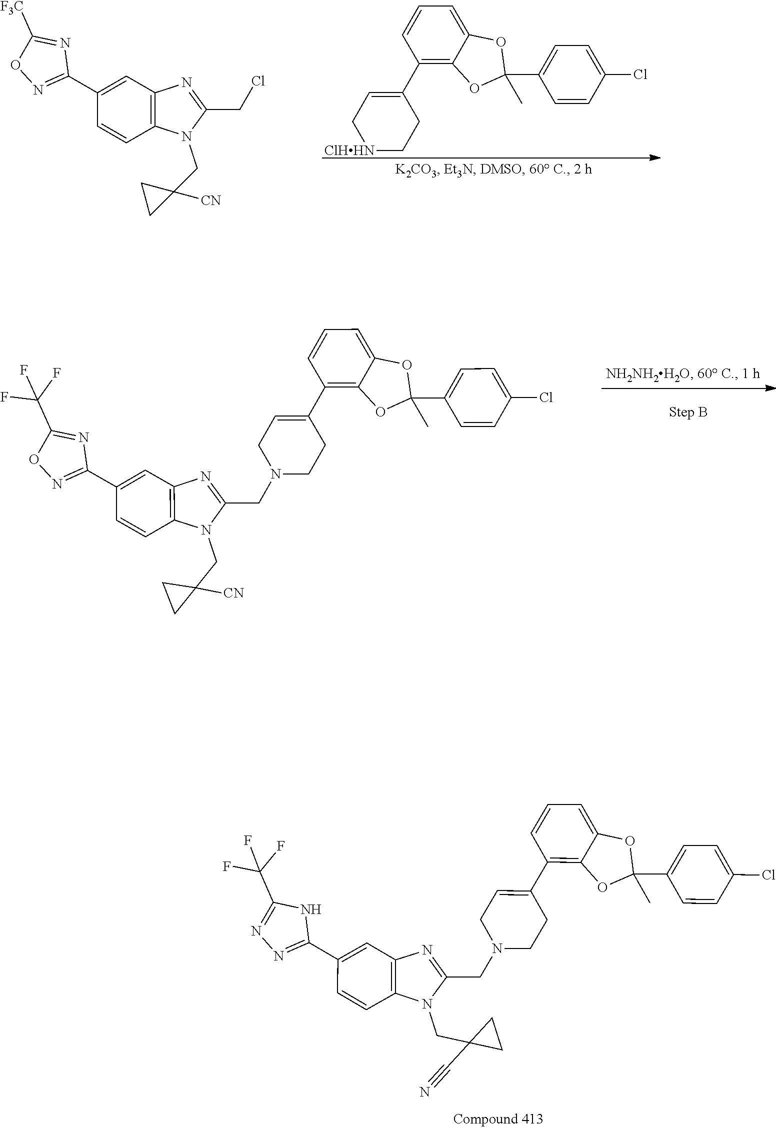

### Step A: 1-((2-((4-(2-(4-chlorophenyl)-2-methylbenzo[d][1,3]dioxol-4-yl)-3,6-dihydropyridin-1(2H)-yl)methyl)-5-(5-(trifluoromethyl)-1,2,4-oxadiazol-3-yl)-1H-benzo[d]imidazol-1-yl)methyl)cyclopropane-1-carbonitrile

A mixture of 4-(2-(4-chlorophenyl)-2-methylbenzo[d][1,3]dioxol-4-yl)-1,2,3,6-tetrahydropyridine hydrochloride (43 mg, 0.13 mmol), K₂CO₃ (54 mg, 0.39 mmol) and Et₃N (66 mg, 0.65 mmol) in DMSO (6 mL) was stirred at 25° C. for 0.5 hour. 1-((2-(chloromethyl)-5-(5-(trifluoromethyl)-1,2,4-oxadiazol-3-yl)-1H-benzo[d]imidazol-1-yl)methyl)cyclopropane-1-carbonitrile (50 mg, 0.13 mmol) was added and the mixture was stirred at 60° C. for 2 hours. The mixture was poured into water (30 mL) and extracted with EA (3×30 mL), the combined organic layers were washed with brine, dried over sodium sulfate, filtered, and concentrated under reduced pressure. The residue was purified by pre-TLC (PE:EA=1:1) to give 1-((2-((4-(2-(4-chlorophenyl)-2-methylbenzo[d][1,3]dioxol-4-yl)-3,6-dihydropyridin-1(2H)-yl)methyl)-5-(5-(trifluoromethyl)-1,2,4-oxadiazol-3-yl)-1H-benzo[d]imidazol-1-yl)methyl)cyclopropane-1-carbonitrile (20 mg, yield: 23%) as a colorless oil. MS Calcd.: 672.2; MS Found: 673.4 [M+H]⁺.

### Step B: 1-{[2-({4-[2-(4-chlorophenyl)-2-methyl-2H-1,3-benzodioxol-4-yl]-1,2,3,6-tetrahydropyridin-1-yl}methyl)-5-[5-(trifluoromethyl)-4H-1,2,4-triazol-3-yl]-1H-1,3-benzodiazol-1-yl]methyl}cyclopropane-1-carbonitrile (Compound 413)

A mixture of 1-((2-((4-(2-(4-chlorophenyl)-2-methylbenzo[d][1,3]dioxol-4-yl)-3,6-dihydropyridin-1(2H)-yl)methyl)-5-(5-(trifluoromethyl)-1,2,4-oxadiazol-3-yl)-1H-benzo[d]imidazol-1-yl)methyl)cyclopropane-1-carbonitrile (20 mg, 0.03 mmol) and NH₂NH₂·H₂O (0.5 mL) in EtOH (2 mL) was stirred at 60° C. for 1 hour under N₂. The reaction mixture was purified by prep-HPLC to give 1-{[2-({4-[2-(4-chlorophenyl)-2-methyl-2H-1,3-benzodioxol-4-yl]-1,2,3,6-tetrahydropyridin-1-yl}methyl)-5-[5-(trifluoromethyl)-4H-1,2,4-triazol-3-yl]-1H-1,3-benzodiazol-1-yl]methyl}cyclopropane-1-carbonitrile Compound 413 (2.1 mg, yield: 10%, FA salt) as white solid.

MS Calcd.: 671.2; MS Found: 672.1 [M+H]⁺.

¹H NMR (400 MHz, MeOD): δ 8.42 (s, 1H), 8.34 (s, 1H), 8.06-8.01 (d, J=10.0 Hz, 1H), 7.90-7.85 (d, J=8.40 Hz, 1H), 7.58-7.54 (d, J=13.6 Hz, 2H), 7.40-7.35 (d, J=13.6 Hz, 2H), 6.85-6.69 (m, 3H), 6.38 (s, 1H), 4.78 (s, 1H), 4.57 (s, 1H), 4.13 (s, 2H), 2.88-2.83 (m, 2H), 2.70-2.54 (m, 2H), 2.22-2.00 (m, 1H), 1.94 (s, 3H), 1.65-1.53 (m, 1H), 1.47-1.41 (s, 4H). ¹⁹F-NMR (377 MHz): −66.74.

### Example 198: 1-{[2-({4-[2-(5-chloropyridin-2-yl)-2-methyl-2H-1,3-benzodioxol-4-yl]piperidin-1-yl}methyl)-5-(5-methyl-4H-1,2,4-triazol-3-yl)-1H-1,3-benzodiazol-1-yl]methyl}cyclopropane-1-carbonitrile (Compound 414)

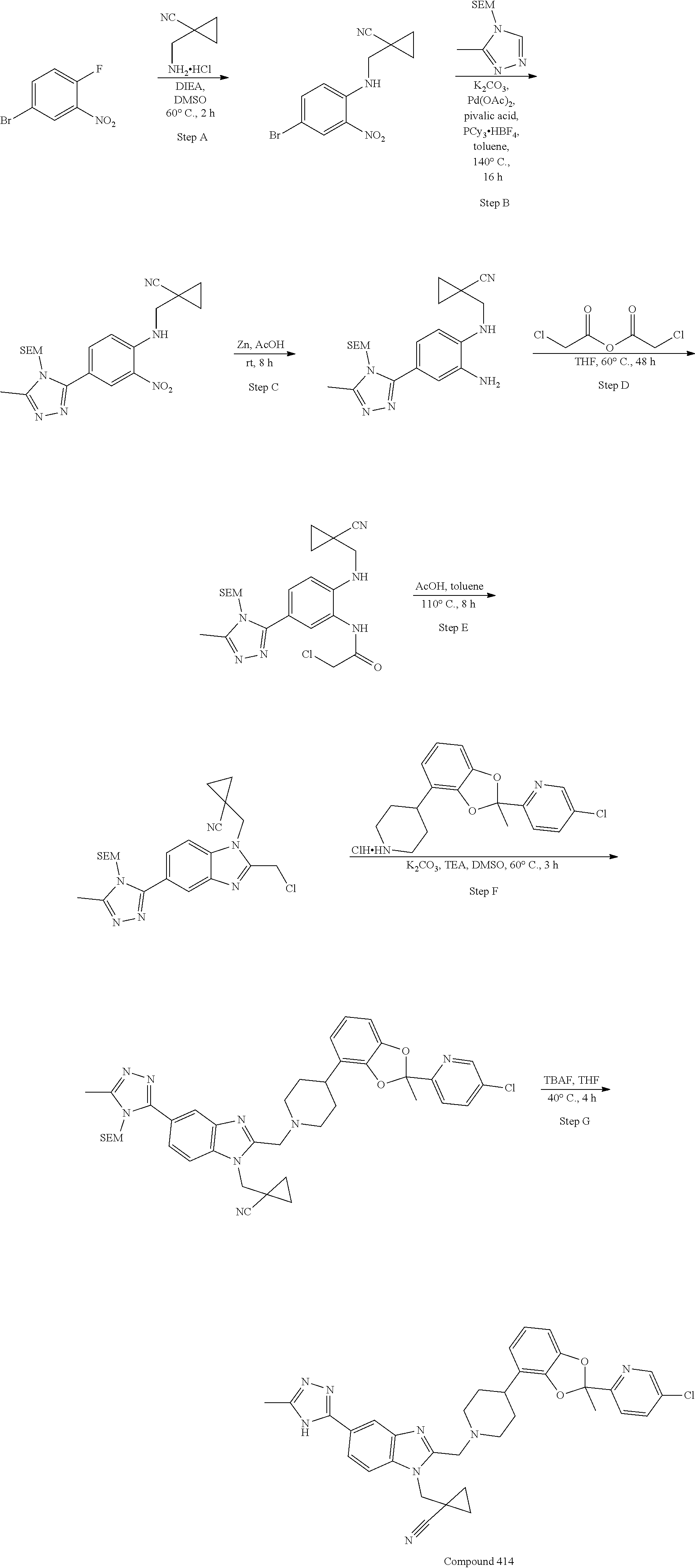

### Step A: 1-(((4-bromo-2-nitrophenyl)amino)methyl)cyclopropanecarbonitrile

A mixture of 1-(aminomethyl)cyclopropanecarbonitrile hydrochloride (200 mg, 1.5 mmol) and DIEA (1.9 g, 14.6 mmol) in DMSO (8 mL) was stirred at room temperature for 10 minutes. 4-Bromo-1-fluoro-2-nitrobenzene (320 mg, 1.5 mmol) was added and the reaction mixture was stirred at 60° C. for 2 hours. The mixture was poured into cold water (20 mL) and extracted with EtOAc (2×20 mL), the combined organic layers were washed with brine, dried over sodium sulfate, filtered and the residue was purified by column chromatography to furnish 1-(((4-bromo-2-nitrophenyl)amino)methyl)cyclopropanecarbonitrile (400 mg, yield: 93%) as yellow solid. MS Calcd.: 295.0; MS Found: 296.1 [M+H]⁺.

### Step B: 1-(((4-(5-methyl-4-((2-(trimethylsilyl)ethoxy)methyl)-4H-1,2,4-triazol-3-yl)-2-nitrophenyl)amino)methyl)cyclopropanecarbonitrile

A mixture of 1-(((4-bromo-2-nitrophenyl)amino)methyl)cyclopropanecarbonitrile (350 mg, 1.2 mmol), 3-methyl-4-((2-(trimethylsilyl)ethoxy)methyl)-4H-1,2,4-triazole (505 mg, 2.4 mmol) (this compound was prepared using the similar procedure as shown in the step C in preparation of Compound 283a, similar case of regio-isomers were observed), pivalic acid (80 mg, 0.78 mmol), K₂CO₃ (982 mg, 7.1 mmol), PCy₃·HBF₄ (87 mg, 0.24 mmol) and Pd(OAc)₂ (27 mg, 0.12 mmol) in toluene (6 mL) was stirred at 140° C. for 16 hours under N₂. The mixture was concentrated under reduced pressure. The residue was purified by column chromatography to furnish 1-(((4-(5-methyl-4-((2-(trimethylsilyl)ethoxy)methyl)-4H-1,2,4-triazol-3-yl)-2-nitrophenyl)amino)methyl)cyclopropanecarbonitrile (105 mg, yield: 21%) as colorless oil. MS Calcd.: 428.2; MS Found: 429.2 [M+H]⁺.

### Step C: 1-(((2-amino-4-(5-methyl-4-((2-(trimethylsilyl)ethoxy)methyl)-4H-1,2,4-triazol-3-yl)phenyl)amino)methyl)cyclopropanecarbonitrile

To a solution of 1-(((4-(5-methyl-4-((2-(trimethylsilyl)ethoxy)methyl)-4H-1,2,4-triazol-3-yl)-2-nitrophenyl)amino)methyl)cyclopropanecarbonitrile (80 mg, 0.19 mmol) in AcOH (6 mL) was added Zn (122 mg, 1.9 mmol). The mixture was stirred at room temperature for 8 hours. The reaction mixture was filtered, and pH value of the mixture was adjusted to 7 with NaHCO₃(aq). The mixture was poured into cold water (10 mL) and extracted with EtOAc (2×10 mL), the combined organic layers were washed with brine, dried over sodium sulfate, filtered and concentrated under reduced pressure to afford 1-(((2-amino-4-(5-methyl-4-((2-(trimethylsilyl)ethoxy)methyl)-4H-1,2,4-triazol-3-yl)phenyl)amino)methyl)cyclopropanecarbonitrile (80 mg, crude, yellow oil) as a crude product, which was used in next step without further purification.

MS Calcd.: 398.2; MS Found: 399.1 [M+H]⁺.

### Step D: 2-chloro-N-(2-(((1-cyanocyclopropyl)methyl)amino)-5-(5-methyl-4-((2-(trimethylsilyl)ethoxy)methyl)-4H-1,2,4-triazol-3-yl)phenyl)acetamide

A mixture of 1-(((2-amino-4-(5-methyl-4-((2-(trimethylsilyl)ethoxy)methyl)-4H-1,2,4-triazol-3-yl) phenyl)amino)methyl)cyclopropanecarbonitrile (80 mg) and 2-chloroacetic anhydride (138 mg, 0.81 mmol) in THE (10 mL) was stirred at 60° C. for 48 hours. The mixture was poured into cold water (10 mL) and extracted with EtOAc (2×10 mL), the combined organic layers were washed with brine, dried over sodium sulfate, filtered and concentrated under reduced pressure. The residue was purified by prep-TLC to furnish 2-chloro-N-(2-(((1-cyanocyclopropyl)methyl)amino)-5-(5-methyl-4-((2-(trimethylsilyl)ethoxy) methyl)-4H-1,2,4-triazol-3-yl)phenyl)acetamide (40 mg, yield: 42%) as yellow oil. MS Calcd.: 474.2; MS Found: 475.2 [M+H]⁺.

### Step E: 1-((2-(chloromethyl)-5-(5-methyl-4-((2-(trimethylsilyl)ethoxy)methyl)-4H-1,2,4-triazol-3-yl)-1H-benzo[d]imidazol-1-yl)methyl)cyclopropanecarbonitrile

To a solution of 2-chloro-N-(2-(((1-cyanocyclopropyl)methyl)amino)-5-(5-methyl-4-((2-(trimethylsilyl)ethoxy)methyl)-4H-1,2,4-triazol-3-yl)phenyl)acetamide (40 mg, 0.07 mmol) in toluene (5 mL) was added AcOH (0.5 mL) and the reaction mixture was stirred at 110° C. for 8 hrs. The pH value of mixture was adjusted to 7 with NaHCO₃(aq). The mixture was poured into cold water (10 mL) and extracted with EtOAc (2×10 mL), the combined organic layers were washed with brine, dried over sodium sulfate, filtered and concentrated under reduced pressure to afford 1-((2-(chloromethyl)-5-(5-methyl-4-((2-(trimethylsilyl)ethoxy)methyl)-4H-1,2,4-triazol-3-yl)-1H-benzo[d]imidazol-1-yl)methyl)cyclopropanecarbonitrile (30 mg, yield: 78%, brown oil) as a crude product, which was used in next steps without further purifications. MS Calcd.: 456.2; MS Found: 657.2 [M+H]⁺.

### Step F: 1-((2-((4-(2-(5-chloropyridin-2-yl)-2-methylbenzo[d][1,3]dioxol-4-yl)piperidin-1-yl) methyl)-5-(5-methyl-4-((2-(trimethylsilyl)ethoxy)methyl)-4H-1,2,4-triazol-3-yl)-1H-benzo[d]imidazol-1-yl)methyl)cyclopropanecarbonitrile

A mixture of 5-chloro-2-(2-methyl-4-(piperidin-4-yl)benzo[d][1,3]dioxol-2-yl)pyridine hydrochloride (24 mg, 0.07 mmol), K₂CO₃ (27 mg, 0.20 mmol) and TEA (20 mg, 0.20 mmol) in DMSO (5 mL) was stirred at room temperature for 10 mins. 1-((2-(chloromethyl)-5-(5-methyl-4-((2-(trimethylsilyl)ethoxy)methyl)-4H-1,2,4-triazol-3-yl)-1H-benzo[d]imidazol-1-yl)methyl)cyclopropanecarbonitrile (100 mg, 0.35 mmol) was added and the reaction mixture was stirred at 60° C. for 3 hours. The mixture was poured into cold water (10 mL) and extracted with EtOAc (2×10 mL), the combined organic layers were washed with brine, dried over sodium sulfate, filtered, and concentrated under reduced pressure. The residue was purified by prep-TLC to furnish 1-((2-((4-(2-(5-chloropyridin-2-yl)-2-methylbenzo[d][1,3]dioxol-4-yl) piperidin-1-yl)methyl)-5-(5-methyl-4-((2-(trimethylsilyl)ethoxy)methyl)-4H-1,2,4-triazol-3-yl)-1H-benzo[d]imidazol-1-yl)methyl)cyclopropanecarbonitrile (40 mg, crude) as yellow solid. MS Calcd.: 750.3; MS Found: 751.3 [M+H]⁺.

### Step G: 1-{[2-({4-[2-(5-chloropyridin-2-yl)-2-methyl-2H-1,3-benzodioxol-4-yl]piperidin-1-yl}methyl)-5-(5-methyl-4H-1,2,4-triazol-3-yl)-1H-1,3-benzodiazol-1-yl]methyl}cyclopropane-1-carbonitrile (Compound 414)

A mixture of 1-((2-((4-(2-(5-chloropyridin-2-yl)-2-methylbenzo[d][1,3]dioxol-4-yl)piperidin-1-yl)methyl)-5-(5-methyl-4-((2-(trimethylsilyl)ethoxy)methyl)-4H-1,2,4-triazol-3-yl)-1H-benzo[d]imidazol-1-yl)methyl)cyclopropanecarbonitrile (40 mg, 0.05 mmol) and TBAF (5 mL, 1M in THF) in THF (1 mL) was stirred at 40° C. for 4 hours. The reaction mixture was purified by prep-HPLC to give 1-{[2-({4-[2-(5-chloropyridin-2-yl)-2-methyl-2H-1,3-benzodioxol-4-yl]piperidin-1-yl}methyl)-5-(5-methyl-4H-1,2,4-triazol-3-yl)-1H-1,3-benzodiazol-1-yl]methyl}cyclopropane-1-carbonitrile Compound 414 (3.7 mg, yield: 11%) as white solid. MS Calcd.: 620.2; MS Found: 621.2 [M+H]⁺.

¹H NMR (400 MHz, MeOD): δ 8.63 (d, J=2.4 Hz, 1H), 8.42 (d, J=0.8 Hz, 1H), 8.06-8.04 (m, 1H), 7.91 (dd, J₁=2.0 Hz, J₂=8.4 Hz, 2H), 7.71 (d, J=8.4 Hz, 1H), 6.89-6.78 (m, 3H), 5.00 (br.s, 1H), 4.67 (s, 2H), 4.03-3.99 (m, 2H), 3.85 (br.s, 1H), 3.50-3.42 (m, 2H), 3.20-3.08 (m, 1H), 2.67 (s, 3H), 2.42-2.20 (m, 2H), 2.18-2.10 (m, 2H), 2.05 (s, 3H), 1.70-1.60 (m, 1H), 1.50-1.38 (m, 3H).

### Example 199: 1-{[2-({4-[2-(5-chloropyridin-2-yl)-2-methyl-2H-1,3-benzodioxol-4-yl]piperidin-1-yl}methyl)-5-[5-(trifluoromethyl)-4H-1,2,4-triazol-3-yl]-1H-1,3-benzodiazol-1-yl]methyl}cyclopropane-1-carbonitrile (Compound 415)

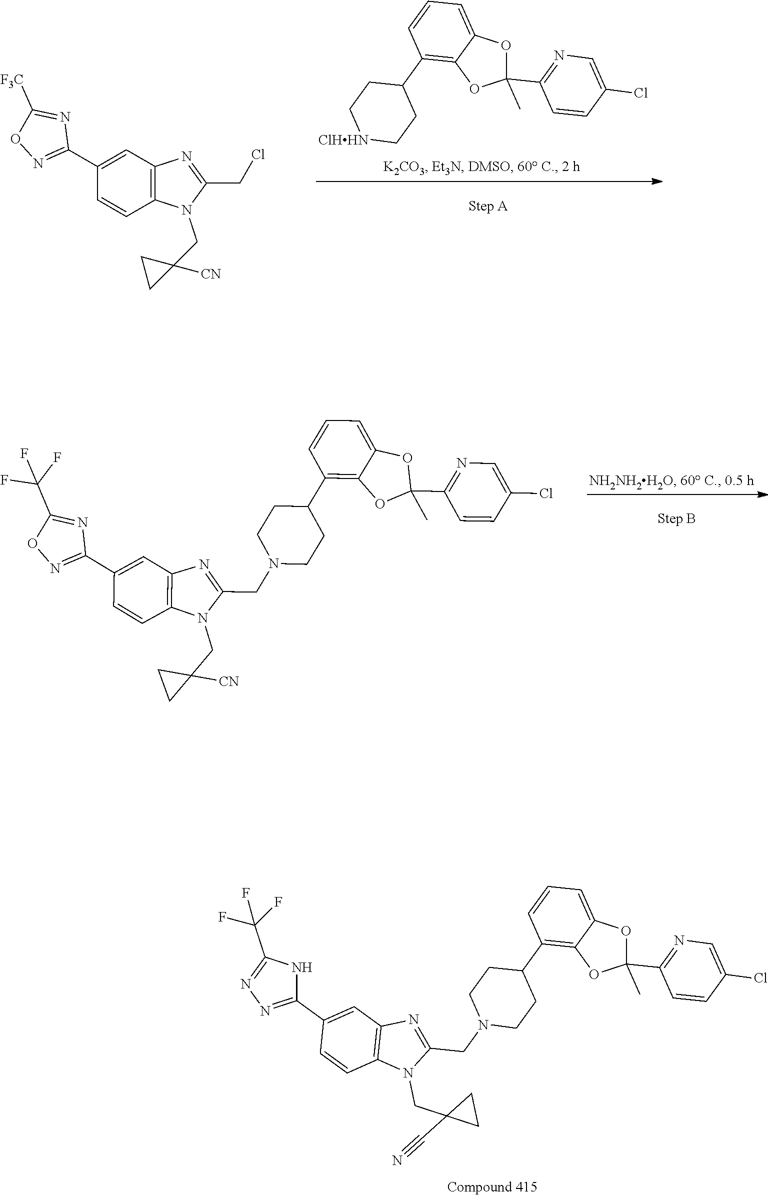

### Step A: 1-((2-((4-(2-(5-chloropyridin-2-yl)-2-methylbenzo[d][1,3]dioxol-4-yl)piperidin-1-yl)methyl)-5-(5-(trifluoromethyl)-1,2,4-oxadiazol-3-yl)-1H-benzo[d]imidazol-1-yl)methyl)cyclopropane-1-carbonitrile

A mixture of 5-chloro-2-(2-methyl-4-(piperidin-4-yl)benzo[d][1,3]dioxol-2-yl)pyridine hydrochloride (20 mg, 0.06 mmol) and K₂CO₃ (25 mg, 0.18 mmol), Et₃N (30 mg, 0.30 mmol) in DMSO (4 mL) was stirred at 25° C. for 0.5 hour. Then 1-((2-(chloromethyl)-5-(5-(trifluoromethyl)-1,2,4-oxadiazol-3-yl)-1H-benzo[d]imidazol-1-yl)methyl)cyclopropane-1-carbonitrile (23 mg, 0.06 mmol) was added and the mixture was stirred at 60° C. for 2 hour. The mixture was poured into water (30 mL) and extracted with EA (3×30 mL), the combined organic layers were washed with brine, dried over sodium sulfate, filtered and concentrated under reduced pressure. The residue was purified by pre-TLC (PE:EA=1:1) to give 1-((2-((4-(2-(5-chloropyridin-2-yl)-2-methylbenzo[d][1,3]dioxol-4-yl)piperidin-1-yl)methyl)-5-(5-(trifluoromethyl)-1,2,4-oxadiazol-3-yl)-1H-benzo[d]imidazol-1 yl)methyl)cyclopropane-1-carbonitrile (20 mg, yield: 49%) as a colorless oil. MS Calcd.: 675.2; MS Found: 676.3 [M+H]⁺.

### Step B: 1-{[2-({4-[2-(5-chloropyridin-2-yl)-2-methyl-2H-1,3-benzodioxol-4-yl]piperidin-1-yl}methyl)-5-[5-(trifluoromethyl)-4H-1,2,4-triazol-3-yl]-1H-1,3-benzodiazol-1-yl]methyl}cyclopropane-1-carbonitrile (Compound 415)

A mixture of 1-((2-((4-(2-(5-chloropyridin-2-yl)-2-methylbenzo[d][1,3]dioxol-4-yl)piperidin-1-yl)methyl)-5-(5-(trifluoromethyl)-1,2,4-oxadiazol-3-yl)-1H-benzo[d]imidazol-1-yl)methyl)cyclopropane-1-carbonitrile (20 mg, 0.03 mmol) and NH₂NH₂·H₂O (0.2 mL) in EtOH (2 mL) was stirred at 60° C. for 0.5 hour under N₂. The reaction mixture was purified by prep-HPLC to give 1-{[2-({4-[2-(5-chloropyridin-2-yl)-2-methyl-2H-1,3-benzodioxol-4-yl]piperidin-1-yl}methyl)-5-[5-(trifluoromethyl)-4H-1,2,4-triazol-3-yl]-1H-1,3-benzodiazol-1-yl]methyl}cyclopropane-1-carbonitrile Compound 415 (2.0 mg, yield: 10%) as white solid.

MS Calcd.: 674.2; MS Found: 675.2 [M+H]⁺.

¹H NMR (400 MHz, MeOD): δ 8.51 (d, J=2.8 Hz, 1H), 8.24 (s, 1H), 7.99-7.94 (m, 1H), 7.77 (dd, J₁=4.8 Hz, J₂=8.4 Hz, 1H), 7.72-7.60 (m, 1H), 7.55 (d, J=9.6 Hz, 1H), 6.70-6.58 (m, 3H), 4.48 (s, 2H), 3.89 (s, 2H), 3.30-3.25 (m, 2H), 2.96-2.90 (m, 2H), 2.68-2.60 (m, 1H), 2.23-2.15 (m, 2H), 2.12-2.02 (m, 5H), 1.93-1.80 (m, 2H), 1.80-1.62 (m, 2H). ¹⁹F-NMR (377 MHz): −65.29.

### Example 200: 2-({4-[2-(5-chloropyridin-2-yl)-2-methyl-2H-1,3-benzodioxol-4-yl]piperidin-1-yl}methyl)-5-(5-cyclopropyl-4H-1,2,4-triazol-3-yl)-1-{[(2S)-oxetan-2-yl]methyl}-1H-1,3-benzodiazole (Compound 270a)

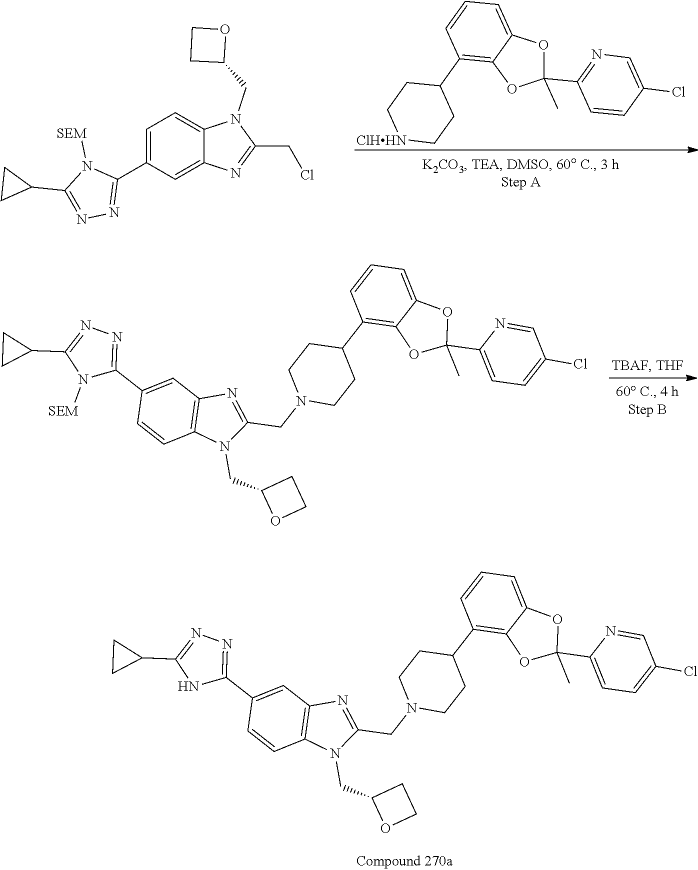

### Step A: 2-((4-(2-(5-chloropyridin-2-yl)-2-methylbenzo[d][1,3]dioxol-4-yl)piperidin-1-yl)methyl)-5-(5-cyclopropyl-4-((2-(trimethylsilyl)ethoxy)methyl)-4H-1,2,4-triazol-3-yl)-1-(((S)-oxetan-2-yl)methyl)-1H-benzo[d]imidazole

A mixture of 5-chloro-2-(2-methyl-4-(piperidin-4-yl)benzo[d][1,3]dioxol-2-yl)pyridine hydrochloride (34 mg, 0.09 mmol), K₂CO₃ (37 mg, 0.27 mmol) and TEA (46 mg, 0.45 mmol) in DMSO (3 mL) was stirred at room temperature for 10 mins. (S)-2-(chloromethyl)-5-(5-cyclopropyl-4-((2-(trimethylsilyl)ethoxy)methyl)-4H-1,2,4-triazol-3-yl)-1-(oxetan-2-ylmethyl)-1H-benzo[d]imidazole (40 mg, 0.085 mmol) was added, and the reaction mixture was stirred at 60° C. for 3 hours. The mixture was poured into cold water (10 mL) and extracted with EtOAc (2×10 mL), the combined organic layers were washed with brine, dried over sodium sulfate, filtered, and concentrated under reduced pressure. The residue was purified by prep-TLC to furnish 2-((4-(2-(5-chloropyridin-2-yl)-2-methylbenzo[d][1,3]dioxol-4-yl)piperidin-1-yl)methyl)-5-(5-cyclopropyl-4-((2-(trimethylsilyl)ethoxy)methyl)-4H-1,2,4-triazol-3-yl)-1-(((S)-oxetan-2-yl)methyl)-1H-benzo[d]imidazole (23 mg, yield: 35%) as a yellow solid. MS Calcd.: 767.3; MS Found: 768.3 [M+H]⁺.

### Step B: 2-({4-[2-(5-chloropyridin-2-yl)-2-methyl-2H-1,3-benzodioxol-4-yl]piperidin-1-yl}methyl)-5-(5-cyclopropyl-4H-1,2,4-triazol-3-yl)-1-{[(2S)-oxetan-2-yl]methyl}-1H-1,3-benzodiazole (Compound 270a)

A mixture of 2-((4-(2-(5-chloropyridin-2-yl)-2-methylbenzo[d][1,3]dioxol-4-yl)piperidin-1-yl)methyl)-5-(5-cyclopropyl-4-((2-(trimethylsilyl)ethoxy)methyl)-4H-1,2,4-triazol-3-yl)-1-(((S)-oxetan-2-yl)methyl)-1H-benzo[d]imidazole (23 mg, 0.03 mmol) and TBAF/THF (1M, 5 mL) in THE (1 mL) was stirred at 60° C. for 4 hours. The reaction mixture was diluted with water and extracted with DCM. The combined organic layers were concentrated and purified by prep-HPLC (0.1% of TFA) to give 2-({4-[2-(5-chloropyridin-2-yl)-2-methyl-2H-1,3-benzodioxol-4-yl]piperidin-1-yl}methyl)-5-(5-cyclopropyl-4H-1,2,4-triazol-3-yl)-1-{[(2S)-oxetan-2-yl]methyl}-1H-1,3-benzodiazole Compound 270a (1.0 mg, yield: 5.4%) as a yellow solid.

MS Calcd.: 637.3; MS Found: 638.3 [M+H]⁺.

¹H NMR (400 MHz, MeOD-d4): 8.63 (t, J=2.0 Hz, 1H), 8.37 (d, J=0.8 Hz, 1H), 8.04 (dd, J₁=1.2 Hz, J2=8.4 Hz, 1H), 7.91 (dd, J₁=2.8 Hz, J₂=8.8 Hz, 1H), 7.74 (d, J=8.4 Hz, 1H), 7.68 (d, J=8.8 Hz, 1H), 6.93-6.74 (m, 3H), 5.30-5.19 (m, 1H), 4.83-4.60 (m, 3H), 4.50-4.40 (m, 1H), 3.91-3.84 (m, 2H), 3.45-3.30 (m, 3H), 3.10-2.95 (m, 1H), 2.85-2.75 (m, 1H), 2.60-2.50 (m, 1H), 2.30-2.00 (m, 9H), 1.18-1.07 (m, 4H).

### Example 201: 2-({4-[2-(5-chloropyridin-2-yl)-2-methyl-2H-1,3-benzodioxol-4-yl]piperidin-1-yl}methyl)-5-(5-methyl-4H-1,2,4-triazol-3-yl)-1-{[(2S)-oxetan-2-yl]methyl}-1H-1,3-benzodiazole (Compound 271a)

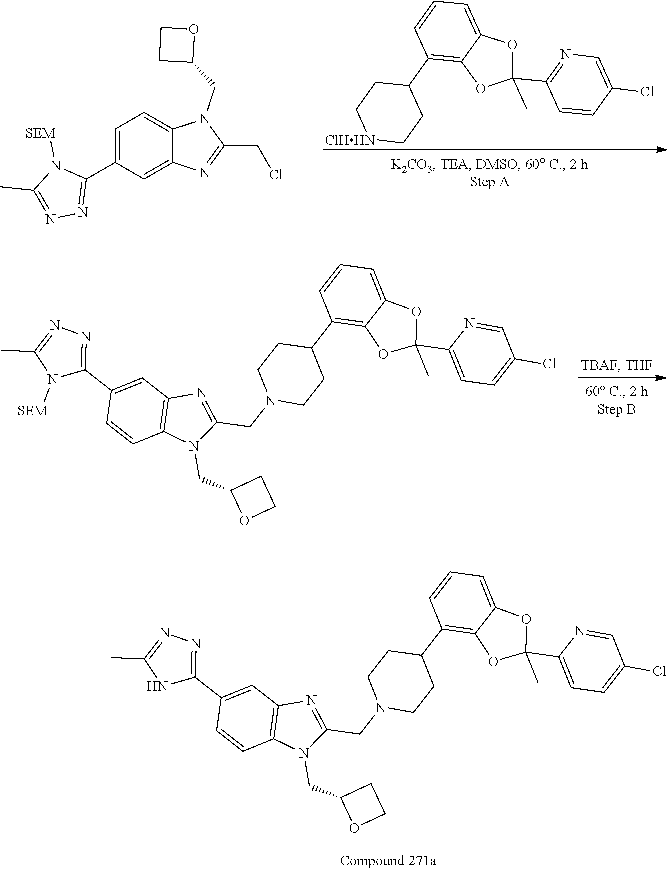

### Step A: 2-((4-(2-(5-chloropyridin-2-yl)-2-methylbenzo[d][1,3]dioxol-4-yl)piperidin-1-yl)methyl)-5-(5-methyl-4-((2-(trimethylsilyl)ethoxy)methyl)-4H-1,2,4-triazol-3-yl)-1-(((S)-oxetan-2-yl)methyl)-1H-benzo[d]imidazole

A mixture of (S)-2-(chloromethyl)-5-(5-methyl-4-((2-(trimethylsilyl)ethoxy)methyl)-4H-1,2,4-triazol-3-yl)-1-(oxetan-2-ylmethyl)-1H-benzo[d]imidazole (17 mg, 0.038 mmol), K₂CO₃ (16 mg, 0.11 mmol) and TEA (12 mg, 0.11 mmol) in DMSO (2 mL) was stirred at rt for 1 hour. 5-chloro-2-(2-methyl-4-(piperidin-4-yl)benzo[d][1,3]dioxol-2-yl)pyridine hydrochloride (13 mg, 0.038 mmol) was added and the mixture was stirred at 60° C. for 2 h. The mixture was purified by column chromatography on silica gel to obtain 2-((4-(2-(5-chloropyridin-2-yl)-2-methylbenzo[d][1,3]dioxol-4-yl)piperidin-1-yl)methyl)-5-(5-methyl-4-((2-(trimethylsilyl)ethoxy)methyl)-4H-1,2,4-triazol-3-yl)-1-(((S)-oxetan-2-yl)methyl)-1H-benzo[d]imidazole (14 mg, yield: 49%) as a white solid. MS Calcd: 741.3; MS Found: 742.3 [M+H]⁺.

### Step B: 2-({4-[2-(5-chloropyridin-2-yl)-2-methyl-2H-1,3-benzodioxol-4-yl]piperidin-1-yl}methyl)-5-(5-methyl-4H-1,2,4-triazol-3-yl)-1-{[(2S)-oxetan-2-yl]methyl}-1H-1,3-benzodiazole (Compound 271a)

A mixture of 2-((4-(2-(5-chloropyridin-2-yl)-2-methylbenzo[d][1,3]dioxol-4-yl)piperidin-1-yl)methyl)-5-(5-methyl-4-((2-(trimethylsilyl)ethoxy)methyl)-4H-1,2,4-triazol-3-yl)-1-(((S)-oxetan-2-yl)methyl)-1H-benzo[d]imidazole (14 mg, 0.019 mmol) in TBAF (in THF, 1M) (1.5 mL) was stirred at 60° C. for 2 hours. The mixture was purified by prep-HPLC to obtain 2-({4-[2-(5-chloropyridin-2-yl)-2-methyl-2H-1,3-benzodioxol-4-yl]piperidin-1-yl}methyl)-5-(5-methyl-4H-1,2,4-triazol-3-yl)-1-{[(2S)-oxetan-2-yl]methyl}-1H-1,3-benzodiazole Compound 271a (2.0 mg, yield: 17%) as a white solid.

MS Calcd: 611.2; MS Found: 612.3 [M+H]⁺.

¹H NMR (400 MHz, MeOD) 8.53 (s, 1H), 8.28 (s, 1H), 7.95 (d, J=8.4 Hz, 1H), 7.80 (dd, J=2.8 Hz, J=8.8 Hz, 1H), 7.64 (d, J=8.8 Hz, 1H), 7.58 (d, J=8.4 Hz, 1H), 6.85-6.63 (m, 3H), 5.20-5.04 (m, 2H), 4.75-4.33 (m, 3H), 3.82-3.73 (m, 2H), 3.35-3.20 (m, 2H), 3.02-2.90 (m, 1H), 2.80-2.66 (m, 1H), 2.50-2.38 (m, 1H), 2.42 (s, 3H), 2.20-1.95 (m, 6H), 1.94 (s, 3H).

### Example 202: 4-[4-(1-{[5-(5-cyclopropyl-4H-1,2,4-triazol-3-yl)-1-{[(2S)-oxetan-2-yl]methyl}-1H-1,3-benzodiazol-2-yl]methyl}piperidin-4-yl)-2-methyl-2H-1,3-benzodioxol-2-yl]-3-fluorobenzonitrile (Compound 277a)

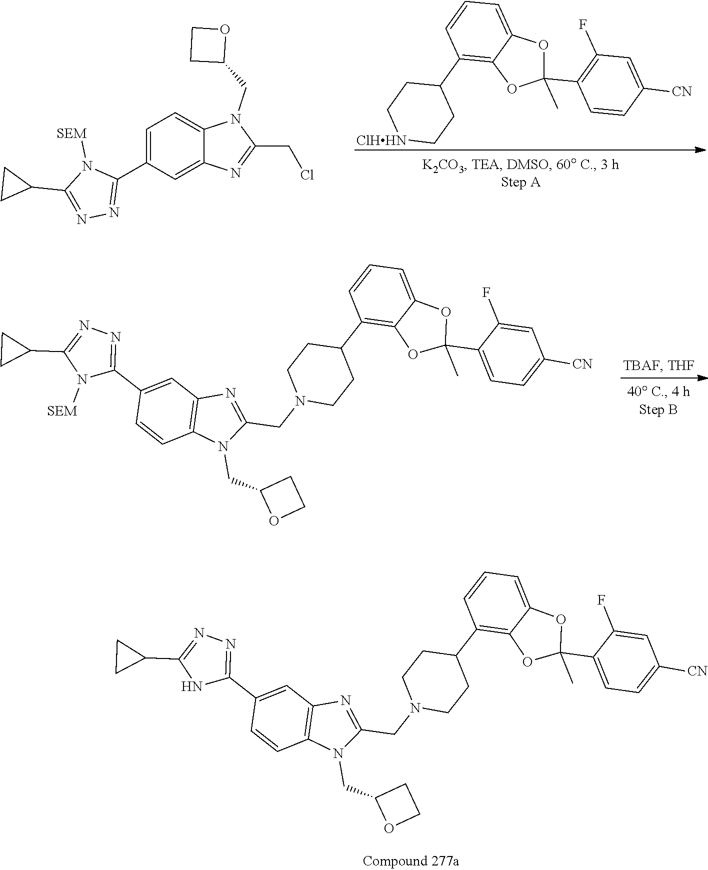

### Step A: 4-(4-(1-((5-(5-cyclopropyl-4-((2-(trimethylsilyl)ethoxy)methyl)-4H-1,2,4-triazol-3-yl)-1-((S)-oxetan-2-ylmethyl)-1H-benzo[d]imidazol-2-yl)methyl)piperidin-4-yl)-2-methylbenzo[d][1,3]dioxol-2-yl)-3-fluorobenzonitrile

A mixture of 3-fluoro-4-(2-methyl-4-(piperidin-4-yl)benzo[d][1,3]dioxol-2-yl)benzonitrile hydrochloride (31 mg, 0.083 mmol), K₂CO₃ (23 mg, 0.16 mmol), TEA (1.0 mL) and (S)-2-(chloromethyl)-5-(5-cyclopropyl-4-((2-(trimethylsilyl)ethoxy)methyl)-4H-1,2,4-triazol-3-yl)-1-(oxetan-2-ylmethyl)-1H-benzo[d]imidazole (43 mg, 0.091 mmol) in DMSO (2.0 mL) was stirred at 60° C. for 3 h. Upon cooling down, the mixture was diluted with H₂O (70 mL) and extracted with EA (15 mL×3). The combined organic layers were washed with brine (25 mL), dried over anhydrous Na₂SO₄, and concentrated under vacuum. The residue was purified by Prep-TLC to furnish 4-(4-(1-((5-(5-cyclopropyl-4-((2-(trimethylsilyl)ethoxy)methyl)-4H-1,2,4-triazol-3-yl)-1-((S)-oxetan-2-ylmethyl)-1H-benzo[d]imidazol-2-yl)methyl)piperidin-4-yl)-2-methylbenzo[d][1,3]dioxol-2-yl)-3-fluorobenzonitrile (20 mg, 31%) as a white solid. MS Calcd.: 775.4; MS Found: 776.3 [M+H]⁺.

### Step B: 4-[4-(1-{[5-(5-cyclopropyl-4H-1,2,4-triazol-3-yl)-1-{[(2S)-oxetan-2-yl]methyl}-1H-1,3-benzodiazol-2-yl]methyl}piperidin-4-yl)-2-methyl-2H-1,3-benzodioxol-2-yl]-3-fluorobenzonitrile (Compound 277a)

A mixture of TBAF (4 mL, 1 M in THF) and 4-(4-(1-((5-(5-cyclopropyl-4-((2-(trimethylsilyl)ethoxy)methyl)-4H-1,2,4-triazol-3-yl)-1-((S)-oxetan-2-ylmethyl)-1H-benzo[d]imidazol-2-yl)methyl)piperidin-4-yl)-2-methylbenzo[d][1,3]dioxol-2-yl)-3-fluorobenzonitrile (20 mg, 0.025 mmol) in THF (1 mL) was stirred at 40° C. for 40 mins. TBAF (2 mL, 1 M in THF) was added, the mixture was stirred at 40° C. for 4 h. Upon cooling down, the mixture was diluted with EA, washed with H₂O and brine, concentrated under vacuum. The residue was diluted with DMF, purified by Prep-HPLC (0.1% TFA, then 0.1% NH3H2O) to furnish 4-[4-(1-{[5-(5-cyclopropyl-4H-1,2,4-triazol-3-yl)-1-{[(2S)-oxetan-2-yl]methyl}-1H-1,3-benzodiazol-2-yl]methyl}piperidin-4-yl)-2-methyl-2H-1,3-benzodioxol-2-yl]-3-fluorobenzonitrile Compound 277a (1.1 mg, yield 6.8%) as a white solid.

MS Calcd.: 645.3; MS Found: 646.4 [M+H]⁺.

¹H NMR (400 MHz, MeOD): δ 8.28-8.21 (m, 1H), 8.00-7.88 (m, 1H), 7.80-7.76 (m, 1H), 7.76-7.68 (m, 1H), 7.67-7.61 (m, 1H), 7.58 (dd, J1=1.2 Hz, J2=8.0 Hz, 1H), 6.81-6.69 (m, 3H), 5.38-5.27 (m, 2H), 4.75-4.68 (m, 1H), 4.66-4.60 (m, 1H), 4.51-4.43 (m, 1H), 4.03-3.98 (m, 1H), 3.92-3.85 (m, 1H), 3.10-3.04 (m, 1H), 2.98-2.90 (m, 1H), 2.88-2.78 (m, 1H), 2.78-2.65 (m, 1H), 2.60-2.50 (m, 1H), 2.38-2.20 (m, 2H), 2.06 (s, 3H), 2.05-2.00 (m, 1H), 2.00-1.73 (m, 4H), 1.72-1.55 (m, 2H), 1.15-1.07 (m, 2H). ¹⁹F-NMR (377 MHz): −112.08, −112.15.

### Example 203: 5-chloro-2-[4-(1-{[6-(5-cyclopropyl-4H-1,2,4-triazol-3-yl)-3-{[(2S)-oxetan-2-yl]methyl}-3H-imidazo[4,5-b]pyridin-2-yl]methyl}piperidin-4-yl)-2-methyl-2H-1,3-benzodioxol-2-yl]pyridine (Compound 268a)

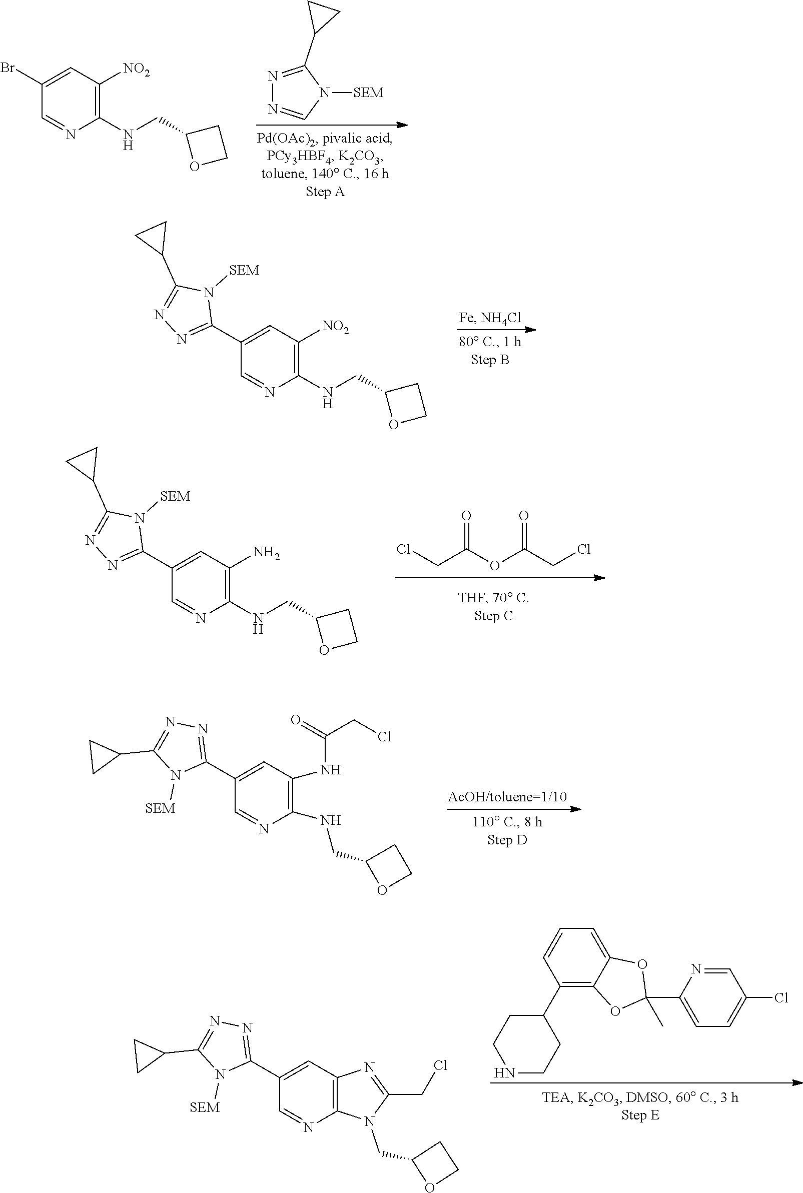

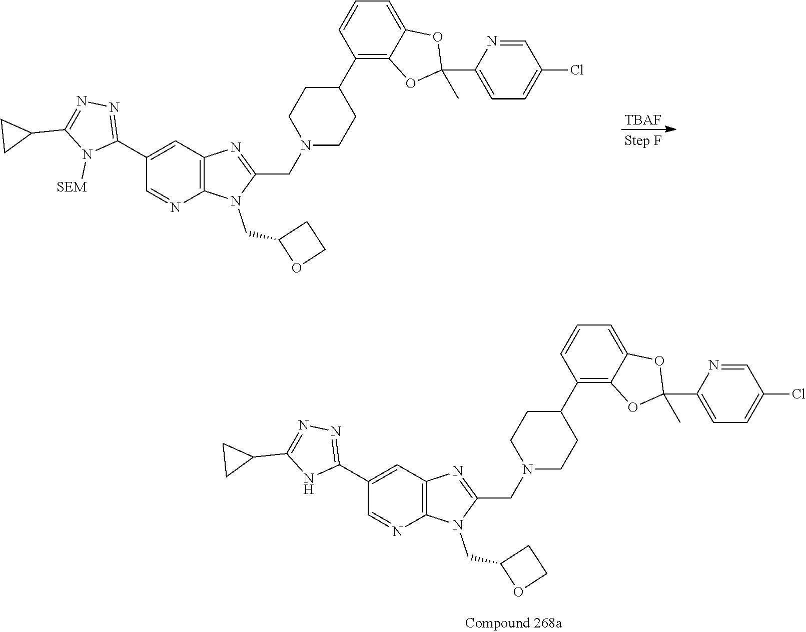

### Step A: (S)-5-(5-cyclopropyl-4-((2-(trimethylsilyl)ethoxy)methyl)-4H-1,2,4-triazol-3-yl)-3-nitro-N-(oxetan-2-ylmethyl)pyridin-2-amine

A mixture of (S)-5-bromo-3-nitro-N-(oxetan-2-ylmethyl)pyridin-2-amine (800 mg, 2.8 mmol), 3-cyclopropyl-4-((2-(trimethylsilyl)ethoxy)methyl)-4H-1,2,4-triazole (1.32 g, 5.6 mmol), Pd(OAc)₂ (63 mg, 0.28 mmol), pivalic acid (187 mg, 1.8 mmol), PCy₃HBF₄ (206 mg, 0.56 mmol) and K₂CO₃ (2.3 g, 16.7 mmol) in toluene (10 mL) was stirred at 140° C. under Ar for 16 hours. Upon cooling down, the mixture was concentrated under vacuum, the residue was purified by Prep-TLC (PE/EA=4/1) to furnish (S)-5-(5-cyclopropyl-4-((2-(trimethylsilyl)ethoxy)methyl)-4H-1,2,4-triazol-3-yl)-3-nitro-N-(oxetan-2-ylmethyl)pyridin-2-amine (1.0 g, crude) as yellow oil. MS Calcd.: 446.2; MS Found: 447.2 [M+H]⁺.

### Step B: (S)-5-(5-cyclopropyl-4-((2-(trimethylsilyl)ethoxy)methyl)-4H-1,2,4-triazol-3-yl)-N2-(oxetan-2-ylmethyl)pyridine-2,3-diamine

A mixture of (S)-5-(5-cyclopropyl-4-((2-(trimethylsilyl)ethoxy)methyl)-4H-1,2,4-triazol-3-yl)-3-nitro-N-(oxetan-2-ylmethyl)pyridin-2-amine (1.02 g, 2.28 mmol) and NH₄Cl (1.22 g, 22.8 mmol) in EtOH/H₂O (10 mL/5 mL) was stirred at 80° C. for 10 min. Fe (638 mg, 11.4 mmol) was added, the mixture was stirred at 80° C. for 1 hr.

Upon cooling down, the mixture was diluted with DCM/MeOH (V/V=10/1), washed with brine, dried over Na₂SO₄, concentrated under vacuum to furnish (S)-5-(5-cyclopropyl-4-((2-(trimethylsilyl)ethoxy)methyl)-4H-1,2,4-triazol-3-yl)-N2-(oxetan-2-ylmethyl)pyridine-2,3-diamine (926 mg, crude) as yellow oil. MS Calcd.: 416.2; MS Found: 417.3 [M+H]⁺.

### Step C: (S)-2-chloro-N-(5-(5-cyclopropyl-4-((2-(trimethylsilyl)ethoxy)methyl)-4H-1,2,4-triazol-3-yl)-2-((oxetan-2-ylmethyl)amino)pyridin-3-yl)acetamide

To a solution of (S)-5-(5-cyclopropyl-4-((2-(trimethylsilyl)ethoxy)methyl)-4H-1,2,4-triazol-3-yl)-N2-(oxetan-2-ylmethyl)pyridine-2,3-diamine (926 mg) in THE (10 mL) was added 2-chloroacetic anhydride (177 mg, 1.04 mmol) dropwisely at 0° C., the mixture was stirred at 70° C. under Ar for 16 hours. The mixture was diluted with EA, washed with aq NaHCO₃ and brine, dried over Na₂SO₄, concentrated to furnish (S)-2-chloro-N-(5-(5-cyclopropyl-4-((2-(trimethylsilyl)ethoxy)methyl)-4H-1,2,4-triazol-3-yl)-2-((oxetan-2-ylmethyl)amino)pyridin-3-yl)acetamide (907 mg, crude) as a brown oil. MS Calcd.: 492.2; MS Found: 493.2 [M+H]⁺.

### Step D: (S)-2-(chloromethyl)-6-(5-cyclopropyl-4-((2-(trimethylsilyl)ethoxy)methyl)-4H-1,2,4-triazol-3-yl)-3-(oxetan-2-ylmethyl)-3H-imidazo[4,5-b]pyridine

To a solution of (S)-2-chloro-N-(5-(5-cyclopropyl-4-((2-(trimethylsilyl)ethoxy)methyl)-4H-1,2,4-triazol-3-yl)-2-((oxetan-2-ylmethyl)amino)pyridin-3-yl)acetamide (907 mg) in toluene (10 mL) was added AcOH (1 mL), the mixture was stirred at 110° C. for 8 hours. The mixture was diluted with EA, washed with aq NaHCO₃ and brine, dried over Na₂SO₄, concentrated, purified by silica gel chromatography (DCM/MeOH=200/1-50/1) to furnish (S)-2-(chloromethyl)-6-(5-cyclopropyl-4-((2-(trimethylsilyl)ethoxy)methyl)-4H-1,2,4-triazol-3-yl)-3-(oxetan-2-ylmethyl)-3H-imidazo[4,5-b]pyridine (100 mg, yield 11%) as a yellow oil. MS Calcd.: 474.2; MS Found: 475.2 [M+H]⁺.

### Step E: 2-((4-(2-(5-chloropyridin-2-yl)-2-methylbenzo[d][1,3]dioxol-4-yl)piperidin-1-yl)methyl)-6-(5-cyclopropyl-4-((2-(trimethylsilyl)ethoxy)methyl)-4H-1,2,4-triazol-3-yl)-3-(((S)-oxetan-2-yl)methyl)-3H-imidazo[4,5-b]pyridine

A mixture of 5-chloro-2-(2-methyl-4-(piperidin-4-yl)benzo[d][1,3]dioxol-2-yl)pyridine (48 mg, 0.13 mmol), TEA (38 mg, 0.38 mmol) and K₂CO₃ (53 mg, 0.38 mmol) in DMSO (3 mL) was stirred at rt for 10 min. (S)-2-(chloromethyl)-6-(5-cyclopropyl-4-((2-(trimethylsilyl)ethoxy)methyl)-4H-1,2,4-triazol-3-yl)-3-(oxetan-2-ylmethyl)-3H-imidazo[4,5-b]pyridine (60 mg, 0.13 mmol) in DMSO (3 mL) was added, the mixture was stirred at 60° C. for 3 hrs. The reaction was diluted with EA, washed with H₂O and brine, dried over sodium sulfate, concentrated in vacuum, purified by prep-TLC (DCM/MeOH=20/1) to give 2-((4-(2-(5-chloropyridin-2-yl)-2-methylbenzo[d][1,3]dioxol-4-yl)piperidin-1-yl)methyl)-6-(5-cyclopropyl-4-((2-(trimethylsilyl)ethoxy)methyl)-4H-1,2,4-triazol-3-yl)-3-(((S)-oxetan-2-yl)methyl)-3H-imidazo[4,5-b]pyridine (58 mg, yield: 59%) as a yellow oil. MS Calcd.: 768.3; MS Found: 769.4 [M+H]⁺.

### Step F: 5-chloro-2-[4-(1-{[6-(5-cyclopropyl-4H-1,2,4-triazol-3-yl)-3-{[(2S)-oxetan-2-yl]methyl}-3H-imidazo[4,5-b]pyridin-2-yl]methyl}piperidin-4-yl)-2-methyl-2H-1,3-benzodioxol-2-yl]pyridine (Compound 268a)

To a solution of 2-((4-(2-(5-chloropyridin-2-yl)-2-methylbenzo[d][1,3]dioxol-4-yl)piperidin-1-yl)methyl)-6-(5-cyclopropyl-4-((2-(trimethylsilyl)ethoxy)methyl)-4H-1,2,4-triazol-3-yl)-3-(((S)-oxetan-2-yl)methyl)-3H-imidazo[4,5-b]pyridine (58 mg, 0.075 mmol) in THE (2 mL) was dropwise added TBAF (1.0 M, 4 mL), the mixture was stirred at 60° C. for 4 hrs. The mixture was diluted with EA, washed with H₂O and brine, dried over Na₂SO₄, concentrated, purified by prep-HPLC (0.1% TFA) to give 5-chloro-2-[4-(1-{[6-(5-cyclopropyl-4H-1,2,4-triazol-3-yl)-3-{[(2S)-oxetan-2-yl]methyl}-3H-imidazo[4,5-b]pyridin-2-yl]methyl}piperidin-4-yl)-2-methyl-2H-1,3-benzodioxol-2-yl]pyridine Compound 268a (6.9 mg, yield 12%) as a white solid.

MS Calcd.: 638.3; MS Found: 639.3[M+H]⁺.

¹H NMR (400 MHz, MeOD): δ 9.09 (d, J=2.0 Hz, 1H), 8.68 (d, J=2.0 Hz, 1H), 8.63 (t, J=1.6 Hz, 1H), 7.90 (dd, J1=2.4 Hz, J2=8.4 Hz, 1H), 7.67 (d, J=8.4 Hz, 1H), 6.90-6.75 (m, 3H), 5.30-5.23 (m, 1H), 4.89 (s, 2H), 4.81-4.74 (m, 1H), 4.72-4.65 (m, 2H), 4.43-4.36 (m, 1H), 4.00-3.86 (m, 2H), 3.48-3.30 (m, 2H), 3.13-3.02 (m, 1H), 2.85-2.75 (m, 1H), 2.60-2.48 (m, 1H), 2.30-2.08 (m, 5H), 2.04 (s, 3H), 1.20-1.08 (m, 4H).

### Example 204: 5-chloro-2-[4-(1-{[6-(5-cyclopropyl-4H-1,2,4-triazol-3-yl)-3-{[(2S)-oxetan-2-yl]methyl}-3H-imidazo[4,5-b]pyridin-2-yl]methyl}-1,2,3,6-tetrahydropyridin-4-yl)-2-methyl-2H-1,3-benzodioxol-2-yl]pyridine (Compound 416a)

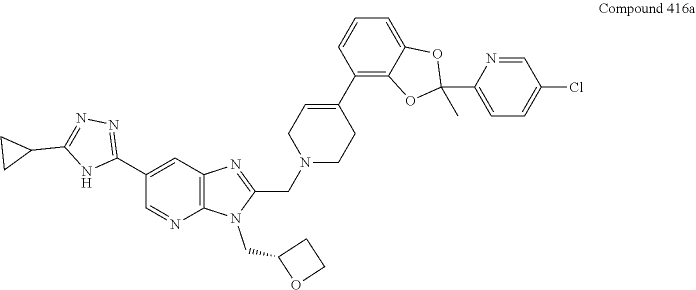 [SMILES: CC1(c2ccc(Cl)cn2)Oc2cccc(C3=CCN(Cc4nc5cc(-c6nnc(C7CC7)[nH]6)cnc5n4C[C@@H]4CCO4)CC3)c2O1]

Starting from 5-chloro-2-[2-methyl-4-(1,2,3,6-tetrahydropyridin-4-yl)-2H-1,3-benzodioxol-2-yl]pyridine, 5-chloro-2-[4-(1-{[6-(5-cyclopropyl-4H-1,2,4-triazol-3-yl)-3-{[(2S)-oxetan-2-yl]methyl}-3H-imidazo[4,5-b]pyridin-2-yl]methyl}-1,2,3,6-tetrahydropyridin-4-yl)-2-methyl-2H-1,3-benzodioxol-2-yl]pyridine Compound 416a (2.5 mg) was obtained as a white solid.

MS Calcd.: 636.2; MS Found: 637.3 [M+H]⁺.

¹H NMR (400 MHz, MeOD): δ9.07 (d, J=2.0 Hz, 1H), 8.67 (d, J=1.6 Hz, 1H), 8.62 (m, J=2.0 Hz, 1H), 7.89 (dd, J1=2.0 Hz, J2=8.4 Hz, 1H), 7.67 (d, J=8.4 Hz, 1H), 6.96-6.85 (m, 3H), 6.44 (br.s, 1H), 5.28-5.20 (m, 2H), 4.72-4.58 (m, 2H), 4.41-4.30 (m, 1H), 4.21-4.14 (m, 2H), 3.82-3.70 (m, 2H), 3.01-2.95 (m, 2H), 2.82-2.70 (m, 1H), 2.58-2.45 (m, 1H), 2.21-2.10 (m, 2H), 2.05 (s, 3H), 1.65-1.55 (m, 1H), 1.20-1.05 (m, 4H).

### Example 205: 4-[4-(1-{[6-(5-cyclopropyl-4H-1,2,4-triazol-3-yl)-3-{[(2S)-oxetan-2-yl]methyl}-3H-imidazo[4,5-b]pyridin-2-yl]methyl}piperidin-4-yl)-2-methyl-2H-1,3-benzodioxol-2-yl]-3-fluorobenzonitrile (Compound 276a)

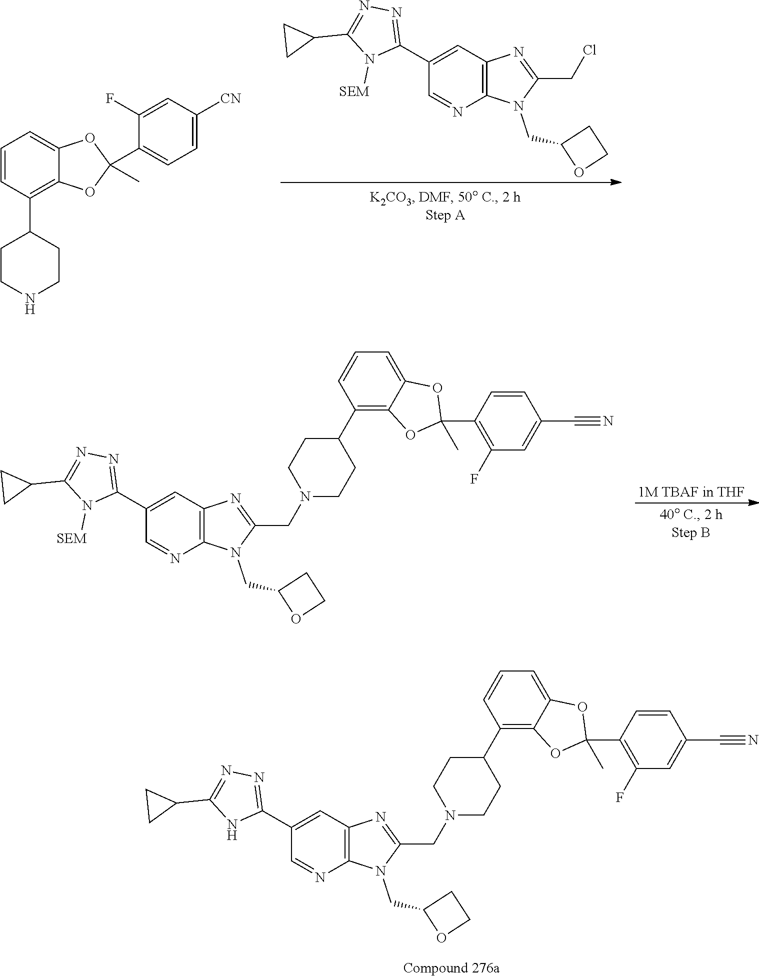 [SMILES: CC1(c2ccc(C#N)cc2F)Oc2cccc(C3CCNCC3)c2O1 ; C[Si](C)(C)CCOCn1c(-c2cnc3c(c2)nc(CCl)n3C[C@@H]2CCO2)nnc1C1CC1 ; CC1(c2ccc(C#N)cc2F)Oc2cccc(C3CCN(Cc4nc5cc(-c6nnc(C7CC7)n6COCC[Si](C)(C)C)cnc5n4C[C@@H]4CCO4)CC3)c2O1 ; CC1(c2ccc(C#N)cc2F)Oc2cccc(C3CCN(Cc4nc5cc(-c6nnc(C7CC7)[nH]6)cnc5n4C[C@@H]4CCO4)CC3)c2O1]

### Step A: 4-(4-(1-((6-(5-cyclopropyl-4-((2-(trimethylsilyl)ethoxy)methyl)-4H-1,2,4-triazol-3-yl)-3-(((S)-oxetan-2-yl)methyl)-3H-imidazo[4,5-b]pyridin-2-yl)methyl)piperidin-4-yl)-2-methylbenzo[d][1,3]dioxol-2-yl)-3-fluorobenzonitrile

To a solution of 3-fluoro-4-(2-methyl-4-(piperidin-4-yl)benzo[d][1,3]dioxol-2-yl)benzamide (TFA salt, 43 mg) in DMF (2.0 mL) was added K₂CO₃ (52 mg, 0.38 mmol) and (S)-2-(chloromethyl)-6-(5-cyclopropyl-4-((2-(trimethylsilyl)ethoxy)methyl)-4H-1,2,4-triazol-3-yl)-3-(oxetan-2-ylmethyl)-3H-imidazo[4,5-b]pyridine (54 mg, 0.11 mmol) The resulting mixture was stirred at 50° C. for 2 hours. The reaction was quenched with water (10 mL), extracted with ethyl acetate (20 mL×3). The organic layers were combined, dried over Na₂SO₄, filtered and concentrated under vacuum. The residue was purified by Prep-TLC (DCM:MeOH=30:1) to give 4-(4-(1-((6-(5-cyclopropyl-4-((2-(trimethylsilyl)ethoxy)methyl)-4H-1,2,4-triazol-3-yl)-3-(((S)-oxetan-2-yl)methyl)-3H-imidazo[4,5-b]pyridin-2-yl)methyl)piperidin-4-yl)-2-methylbenzo[d][1,3]dioxol-2-yl)-3-fluorobenzonitrile (40 mg) as white solid. MS Calcd.: 776.4; MS Found: 389.4 [M/2+H]⁺.

### Step B: 4-[4-(1-{[6-(5-cyclopropyl-4H-1,2,4-triazol-3-yl)-3-{[(2S)-oxetan-2-yl]methyl}-3H-imidazo[4,5-b]pyridin-2-yl]methyl}piperidin-4-yl)-2-methyl-2H-1,3-benzodioxol-2-yl]-3-fluorobenzonitrile (Compound 276a)

A solution of 4-(4-(1-((6-(5-cyclopropyl-4-((2-(trimethylsilyl)ethoxy)methyl)-4H-1,2,4-triazol-3-yl)-3-(((S)-oxetan-2-yl)methyl)-3H-imidazo[4,5-b]pyridin-2-yl)methyl)piperidin-4-yl)-2-methylbenzo[d] [1,3]dioxol-2-yl)-3-fluorobenzonitrile (40 mg, 0.05 mmol) in 1M TBAF in THE (2.0 mL) was heated to 40° C. for 2 h. The reaction mixture was directly purified by Prep-HPLC to afford 4-[4-(1-{[6-(5-cyclopropyl-4H-1,2,4-triazol-3-yl)-3-{[(2S)-oxetan-2-yl]methyl}-3H-imidazo[4,5-b]pyridin-2-yl]methyl}piperidin-4-yl)-2-methyl-2H-1,3-benzodioxol-2-yl]-3-fluorobenzonitrile Compound 276a (2.1 mg, yield: 6.3%) as a white solid.

MS Calcd.: 646.3; MS Found: 647.4 [M+H]⁺.

¹H NMR (400 MHz, DMSO-d₆): δ 9.02 (s, 1H), 8.57 (s, 1H), 7.78 (t, J=8.0 Hz, 1H), 7.64 (d, J=10.8 Hz, 1H), 7.55-7.59 (m, 1H), 6.70-6.81 (m, 3H), 5.28-5.38 (m, 1H), 4.93-5.02 (m, 1H), 4.54-4.66 (m, 2H), 4.42-4.50 (m, 1H), 4.03-4.12 (m, 2H), 3.02-3.15 (m, 2H), 2.70-2.84 (m, 2H), 2.50-2.62 (m, 1H), 2.32-2.44 (m, 2H), 2.08-2.20 (m, 1H), 2.06 (s, 3H), 1.76-2.03 (m, 4H), 1.04-1.20 (m, 4H). ¹⁹F-NMR (377 MHz): −112.10.

### Example 206: 4-[(2S)-2-(4-chloro-2-fluorophenyl)-2-methyl-2H-1,3-benzodioxol-4-yl]-1-{[6-(5-cyclopropyl-4H-1,2,4-triazol-3-yl)-3-{[(2S)-oxetan-2-yl]methyl}-3H-imidazo[4,5-b]pyridin-2-yl]methyl}piperidine (Compound 282a)

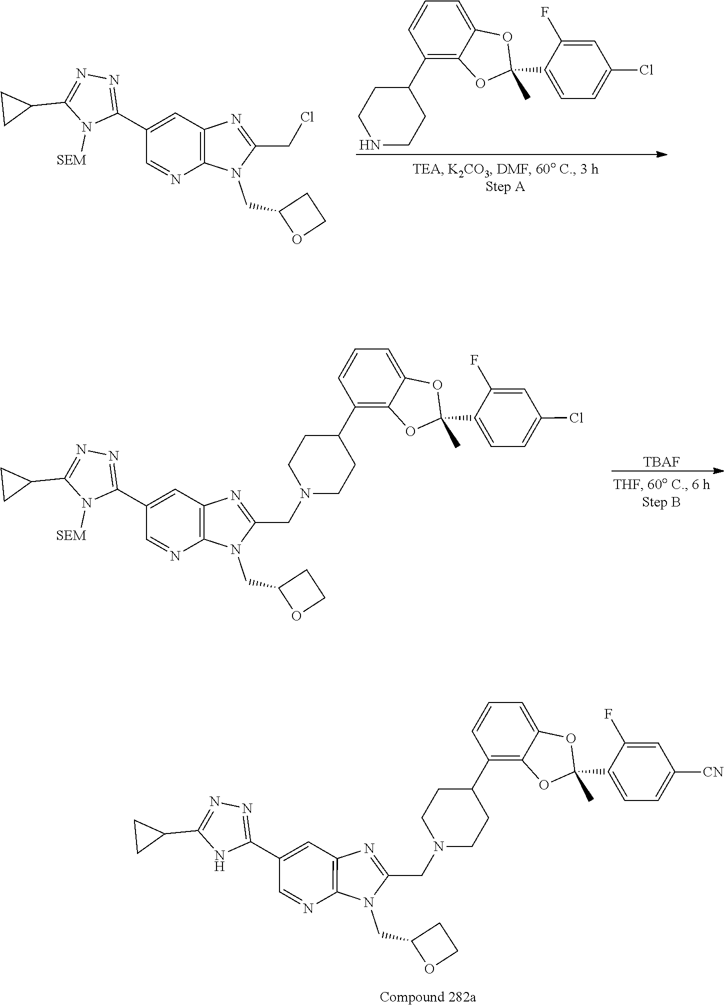 [SMILES: C[Si](C)(C)CCOCn1c(-c2cnc3c(c2)nc(CCl)n3C[C@@H]2CCO2)nnc1C1CC1 ; C[C@]1(c2ccc(Cl)cc2F)Oc2cccc(C3CCNCC3)c2O1 ; C[C@]1(c2ccc(Cl)cc2F)Oc2cccc(C3CCN(Cc4nc5cc(-c6nnc(C7CC7)n6COCC[Si](C)(C)C)cnc5n4C[C@@H]4CCO4)CC3)c2O1 ; "TBAF" ; C[C@]1(c2ccc(C#N)cc2F)Oc2cccc(C3CCN(Cc4nc5cc(-c6nnc(C7CC7)[nH]6)cnc5n4C[C@@H]4CCO4)CC3)c2O1]

### Step A: 2-((4-((S)-2-(4-chloro-2-fluorophenyl)-2-methylbenzo[d][1,3]dioxol-4-yl)piperidin-1-yl)methyl)-6-(5-cyclopropyl-4-((2-(trimethylsilyl)ethoxy)methyl)-4H-1,2,4-triazol-3-yl)-3-(((S)-oxetan-2-yl)methyl)-3H-imidazo[4,5-b]pyridine

A mixture of (S)-4-(2-(4-chloro-2-fluorophenyl)-2-methylbenzo[d][1,3]dioxol-4-yl)piperidine TsOH salt (219 mg, 0.42 mmol), K₂CO₃ (174 mg, 1.26 mmol) and TEA (127 mg, 1.26 mmol) in DMSO (4 ml) was stirred at rt for 10 mins, (S)-2-(chloromethyl)-6-(5-cyclopropyl-4-((2-(trimethylsilyl)ethoxy)methyl)-4H-1,2,4-triazol-3-yl)-3-(oxetan-2-ylmethyl)-3H-imidazo[4,5-b]pyridine (200 mg, 0.42 mmol) was added, the mixture was stirred at 60° C. for 3 h. Upon cooling down, the mixture was diluted with EA, washed with aq NaHCO₃ and brine, dried over anhydrous Na₂SO₄, concentrated under vacuum, purified by Prep-TLC (DCM/MeOH=20/1) to furnish 2-((4-((S)-2-(4-chloro-2-fluorophenyl)-2-methylbenzo[d][1,3]dioxol-4-yl)piperidin-1-yl)methyl)-6-(5-cyclopropyl-4-((2-(trimethylsilyl)ethoxy)methyl)-4H-1,2,4-triazol-3-yl)-3-(((S)-oxetan-2-yl)methyl)-3H-imidazo[4,5-b]pyridine (149 mg, crude) as a yellow oil. MS Calcd.: 785.3; MS Found: 786.4 [M+H]⁺.

### Step B: 4-[(2S)-2-(4-chloro-2-fluorophenyl)-2-methyl-2H-1,3-benzodioxol-4-yl]-1-{[6-(5-cyclopropyl-4H-1,2,4-triazol-3-yl)-3-{[(2S)-oxetan-2-yl]methyl}-3H-imidazo[4,5-b]pyridin-2-yl]methyl}piperidine (Compound 282a)

To a solution of 2-((4-((S)-2-(4-chloro-2-fluorophenyl)-2-methylbenzo[d][1,3]dioxol-4-yl)piperidin-1-yl)methyl)-6-(5-cyclopropyl-4-((2-(trimethylsilyl)ethoxy)methyl)-4H-1,2,4-triazol-3-yl)-3-(((S)-oxetan-2-yl)methyl)-3H-imidazo[4,5-b]pyridine (149 mg, 0.19 mmol) in THF (5 mL) was dropwise added TBAF (1.9 mL, 1.9 mmol, 1 M in THF) at rt, then the mixture was stirred at 60° C. for 4 hrs. TBAF (1 mL, 1 M in THF) was added, the mixture was stirred at 60° C. for 2 hrs. Upon cooling down, the mixture was diluted with EA, washed with H₂O and brine, concentrated under vacuum, purified by Prep-HPLC (0.1% FA) to furnish 4-[(2S)-2-(4-chloro-2-fluorophenyl)-2-methyl-2H-1,3-benzodioxol-4-yl]-1-{[6-(5-cyclopropyl-4H-1,2,4-triazol-3-yl)-3-{[(2S)-oxetan-2-yl]methyl}-3H-imidazo[4,5-b]pyridin-2-yl]methyl}piperidine Compound 282a (2.6 mg, yield 2.1%) as a white solid.

MS Calcd.: 655.2; MS Found: 656.2 [M+H]⁺.

¹H NMR (400 MHz, MeOD): δ 9.01 (d, J=1.6 Hz, 1H), 8.57-8.55 (m, 1H), 7.60 (t, J=8.4 Hz, 1H), 7.28 (dd, J1=2.0 Hz, J2=10.8 Hz, 1H), 7.21 (dd, J1=1.2 Hz, J2=8.4 Hz, 1H), 6.80-6.68 (m, 3H), 5.36-5.30 (m, 2H), 4.66-4.59 (m, 2H), 4.50-4.42 (m, 1H), 4.08-3.98 (m, 2H), 3.10-2.98 (m, 2H), 2.85-2.65 (m, 2H), 2.61-2.53 (m, 2H), 2.39-2.28 (m, 2H), 2.02 (s, 3H), 2.00-1.89 (m, 2H), 1.88-1.75 (m, 2H), 1.18-1.08 (m, 4H). ¹⁹F-NMR (377 MHz): −112.37.

### Example 207: 4-[(2S)-2-(4-chloro-2-fluorophenyl)-2-methyl-2H-1,3-benzodioxol-4-yl]-1-{[6-(5-methyl-4H-1,2,4-triazol-3-yl)-3-{[(2S)-oxetan-2-yl]methyl}-3H-imidazo[4,5-b]pyridin-2-yl]methyl}piperidine (Compound 292a)

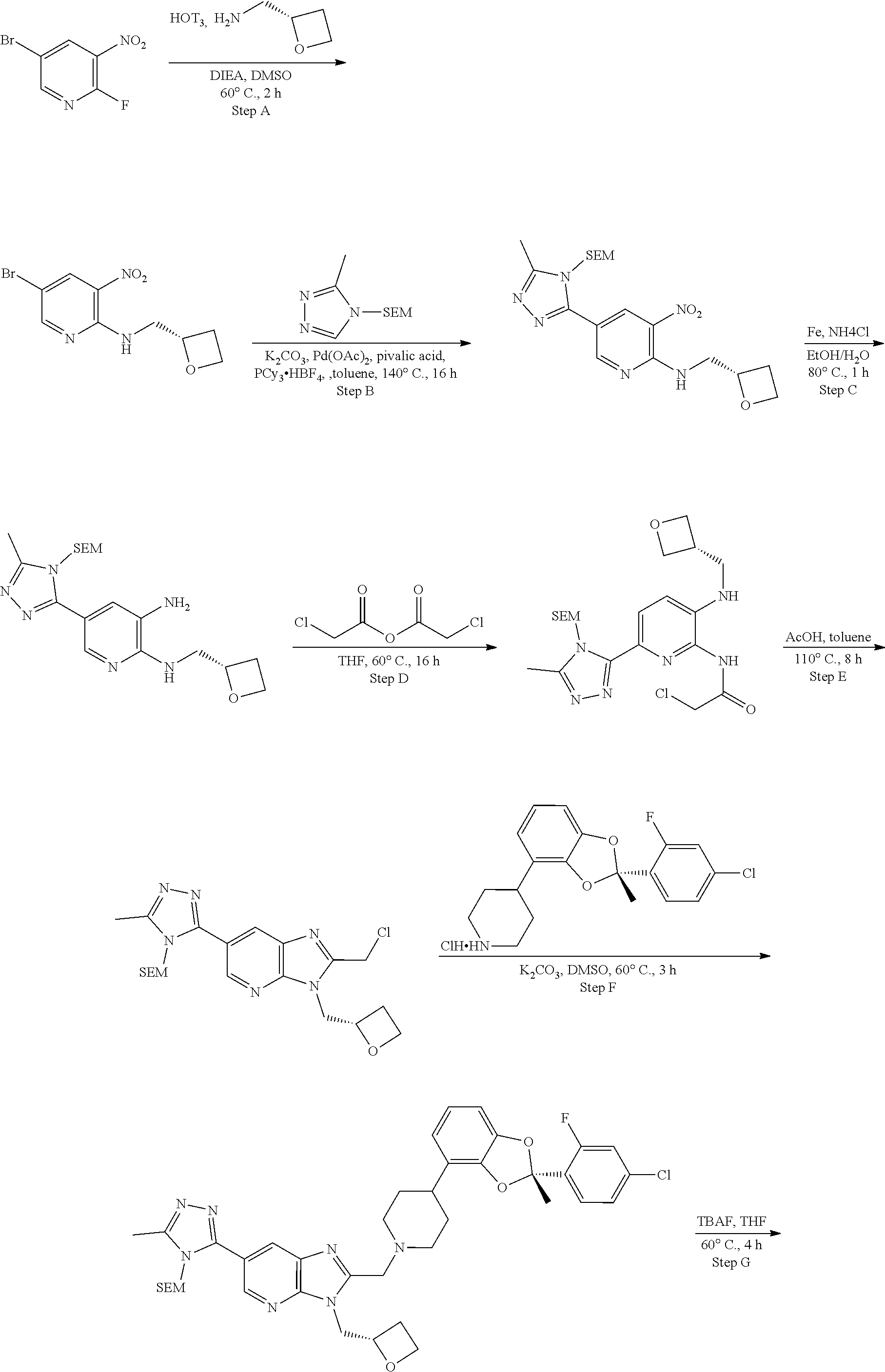

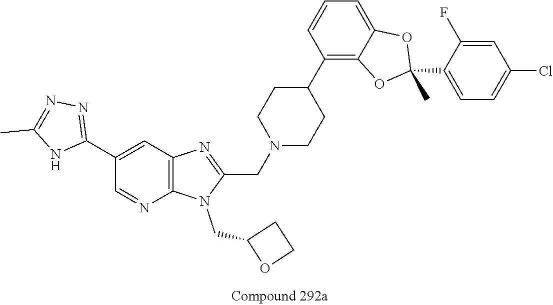 [SMILES: Cc1nnc(-c2cnc3c(c2)nc(CN2CCC(c4cccc5c4O[C@@](C)(c4ccc(Cl)cc4F)O5)CC2)n3C[C@@H]2CCO2)[nH]1]

### Step A: (S)-5-bromo-3-nitro-N-(oxetan-2-ylmethyl)pyridin-2-amine

A mixture of (S)-oxetan-2-ylmethanamine 4-methylbenzenesulfonate (5.0 g, 22.6 mmol) and DIEA (18.6 mL, 113.1 mmol) in DMSO (25 mL) was stirred at room temperature for 10 mins. 5-bromo-2-fluoro-3-nitropyridine (5.9 g, 22.6 mmol) was added and the reaction mixture was stirred at 60° C. for 2 hours. The mixture was poured into cold water (50 mL) and extracted with EtOAc (2×50 mL), the combined organic layer was washed with brine, dried over sodium sulfate, filtered and the residue was purified by column chromatography to furnish (S)-5-bromo-3-nitro-N-(oxetan-2-ylmethyl)pyridin-2-amine (6.0 g, yield: 92%) as yellow solid. MS Calcd.: 287.0; MS Found: 288.0 [M+H]⁺.

### Step B: (S)-5-(5-methyl-4-((2-(trimethylsilyl)ethoxy)methyl)-4H-1,2,4-triazol-3-yl)-3-nitro-N-(oxetan-2-ylmethyl)pyridin-2-amine

A mixture of (S)-5-bromo-3-nitro-N-(oxetan-2-ylmethyl)pyridin-2-amine (800 mg, 2.8 mmol), 3-methyl-4-((2-(trimethylsilyl)ethoxy)methyl)-4H-1,2,4-triazole (1.2 g, 5.6 mmol), pivalic acid (188 mg, 1.8 mmol), K₂CO₃ (2.3 g, 16.7 mmol), PCy₃·HBF₄ (210 mg, 0.56 mmol) and Pd(OAc)₂ (63 mg, 0.28 mmol) in toluene (15 mL) was stirred at 140° C. for 16 hours under N₂. The mixture was concentrated under reduced pressure. The residue was purified by column chromatography to furnish (S)-5-(5-methyl-4-((2-(trimethylsilyl)ethoxy)methyl)-4H-1,2,4-triazol-3-yl)-3-nitro-N-(oxetan-2-ylmethyl)pyridin-2-amine (100 mg, yield: 8.5%) as yellow solid. MS Calcd.: 420.2; MS Found: 421.2 [M+H]⁺.

### Step C: (S)-5-(5-methyl-4-((2-(trimethylsilyl)ethoxy)methyl)-4H-1,2,4-triazol-3-yl)-N2-(oxetan-2-ylmethyl)pyridine-2,3-diamine

To a solution of (S)-5-(5-methyl-4-((2-(trimethylsilyl)ethoxy)methyl)-4H-1,2,4-triazol-3-yl)-3-nitro-N-(oxetan-2-ylmethyl)pyridin-2-amine (100 mg, 0.24 mmol) in EtOH (5 mL) and H₂O (1 mL) was added NH₄Cl (130 mg, 2.4 mmol) and Fe (70 mg, 1.19 mmol). The mixture was stirred at 80° C. for 1 hour. The reaction mixture was filtered and concentrated under reduced pressure to give (S)-5-(5-methyl-4-((2-(trimethylsilyl)ethoxy)methyl)-4H-1,2,4-triazol-3-yl)-N2-(oxetan-2-ylmethyl)pyridine-2,3-diamine (100 mg, crude) as brown oil, which was used in next steps without further purifications. MS Calcd.: 390.2; MS Found: 391.3 [M+H]⁺.

### Step D: 2-chloro-N-(5-(5-methyl-4-((2-(trimethylsilyl)ethoxy)methyl)-4H-1,2,4-triazol-3-yl)-2-((oxetan-3-ylmethyl)amino)phenyl)acetamide

A mixture of (S)-5-(5-methyl-4-((2-(trimethylsilyl)ethoxy)methyl)-4H-1,2,4-triazol-3-yl)-N2-(oxetan-2-ylmethyl)pyridine-2,3-diamine (94 mg) and 2-chloroacetic anhydride (65 mg, 0.17 mmol) in THE (10 mL) was stirred at 60° C. for 16 hours. The mixture was poured into cold water (10 mL) and extracted with EtOAc (2×10 mL), the combined organic layers were washed with brine, dried over sodium sulfate, filtered and concentrated under reduced pressure to give 2-chloro-N-(5-(5-methyl-4-((2-(trimethylsilyl)ethoxy)methyl)-4H-1,2,4-triazol-3-yl)-2-((oxetan-3-ylmethyl)amino)phenyl)acetamide (111 mg, crude) as brown oil, which was used in next steps without further purifications. MS Calcd.: 466.2; MS Found: 467.2 [M+H]⁺.

### Step E: (S)-2-(chloromethyl)-6-(5-methyl-4-((2-(trimethylsilyl)ethoxy)methyl)-4H-1,2,4-triazol-3-yl)-3-(oxetan-2-ylmethyl)-3H-imidazo[4,5-b]pyridine

To a solution of 2-chloro-N-(5-(5-methyl-4-((2-(trimethylsilyl)ethoxy)methyl)-4H-1,2,4-triazol-3-yl)-2-((oxetan-3-ylmethyl)amino)phenyl)acetamide (111 mg) in toluene (10 mL) was added AcOH (1 mL) and the reaction mixture was stirred at 110° C. for 8 hours. The pH value of the mixture was adjusted to 7 with NaHCO₃(aq). The mixture was poured into cold water (10 mL) and extracted with EtOAc (2×10 mL), the combined organic layers were washed with brine, dried over sodium sulfate, filtered and concentrated under reduced pressure to give (S)-2-(chloromethyl)-6-(5-methyl-4-((2-(trimethylsilyl)ethoxy)methyl)-4H-1,2,4-triazol-3-yl)-3-(oxetan-2-ylmethyl)-3H-imidazo[4,5-b]pyridine (120 mg, crude) as colorless oil, which was used in next steps without further purifications. MS Calcd.: 448.2; MS Found: 449.2 [M+H]⁺.

### Step F: 2-((4-((S)-2-(4-chloro-2-fluorophenyl)-2-methylbenzo[d][1,3]dioxol-4-yl)piperidin-1-yl)methyl)-6-(5-methyl-4-((2-(trimethylsilyl)ethoxy)methyl)-4H-1,2,4-triazol-3-yl)-3-((S)-oxetan-2-ylmethyl)-3H-imidazo[4,5-b]pyridine

A mixture of (S)-4-(2-(4-chloro-2-fluorophenyl)-2-methylbenzo[d][1,3]dioxol-4-yl)piperidine 4-methylbenzenesulfonate (46 mg), K₂CO₃ (37 mg, 0.27 mmol) and TEA (27 mg, 0.27 mmol) in DMSO (3 mL) was stirred at room temperature for 10 mins. (S)-2-(chloromethyl)-6-(5-methyl-4-((2-(trimethylsilyl)ethoxy)methyl)-4H-1,2,4-triazol-3-yl)-3-(oxetan-2-ylmethyl)-3H-imidazo[4,5-b]pyridine (40 mg, 0.09 mmol) was added, and the reaction mixture was stirred at 60° C. for 3 hours. The mixture was poured into cold water (10 mL) and extracted with EtOAc (2×10 mL), the combined organic layers were washed with brine, dried over sodium sulfate, filtered and concentrated under reduced pressure. The residue was purified by prep-TLC to furnish 2-((4-((S)-2-(4-chloro-2-fluorophenyl)-2-methylbenzo[d][1,3]dioxol-4-yl) piperidin-1-yl)methyl)-6-(5-methyl-4-((2-(trimethylsilyl)ethoxy)methyl)-4H-1,2,4-triazol-3-yl)-3-((S)-oxetan-2-ylmethyl)-3H-imidazo[4,5-b]pyridine (40 mg, yield: 59%) as yellow solid. MS Calcd.: 759.3; MS Found: 760.2 [M+H]⁺.

### Step G: 4-[(2S)-2-(4-chloro-2-fluorophenyl)-2-methyl-2H-1,3-benzodioxol-4-yl]-1-{[6-(5-methyl-4H-1,2,4-triazol-3-yl)-3-{[(2S)-oxetan-2-yl]methyl}-3H-imidazo[4,5-b]pyridin-2-yl]methyl}piperidine (Compound 292a)

A mixture of 2-((4-((S)-2-(4-chloro-2-fluorophenyl)-2-methylbenzo[d][1,3]dioxol-4-yl)piperidin-1-yl)methyl)-6-(5-methyl-4-((2-(trimethylsilyl)ethoxy)methyl)-4H-1,2,4-triazol-3-yl)-3-((S)-oxetan-2-ylmethyl)-3H-imidazo[4,5-b]pyridine (40 mg, 0.05 mmol) and TBAF (5 mL, 1M in THF) in THE (1 mL) was stirred at 60° C. for 4 hours. The reaction mixture was purified by prep-HPLC to give 4-[(2S)-2-(4-chloro-2-fluorophenyl)-2-methyl-2H-1,3-benzodioxol-4-yl]-1-{[6-(5-methyl-4H-1,2,4-triazol-3-yl)-3-{[(2S)-oxetan-2-yl]methyl}-3H-imidazo[4,5-b]pyridin-2-yl]methyl}piperidine Compound 292a (2.6 mg, yield: 7.9%) as white solid.

MS Calcd.: 629.2; MS Found: 630.2 [M+H]⁺.

¹H NMR (400 MHz, MeOD): δ 9.03 (s, 1H), 8.58 (s, 1H), 7.58 (t, J=8.0 Hz, 1H), 7.27 (dd, J₁=2.0 Hz, J₂=10.8 Hz, 1H), 7.20 (dd, J₁=1.6 Hz, J₂=8.4 Hz, 1H), 6.80-6.68 (m, 3H), 5.37-5.28 (m, 1H), 4.68-4.53 (m, 3H), 4.50-4.43 (m, 1H), 4.12-4.01 (m, 2H), 3.15-3.00 (m, 2H), 2.85-2.68 (m, 2H), 2.65-2.50 (m, 4H), 2.45-2.32 (m, 2H), 2.03 (s, 3H), 2.00-1.90 (m, 2H), 1.90-1.78 (m, 2H). ¹⁹F-NMR (377 MHz): −112.37.

### Example 208: 5-chloro-2-[2-methyl-4-(1-{[6-(5-methyl-4H-1,2,4-triazol-3-yl)-3-{[(2S)-oxetan-2-yl]methyl}-3H-imidazo[4,5-b]pyridin-2-yl]methyl}piperidin-4-yl)-2H-1,3-benzodioxol-2-yl]pyridine (Compound 269a)

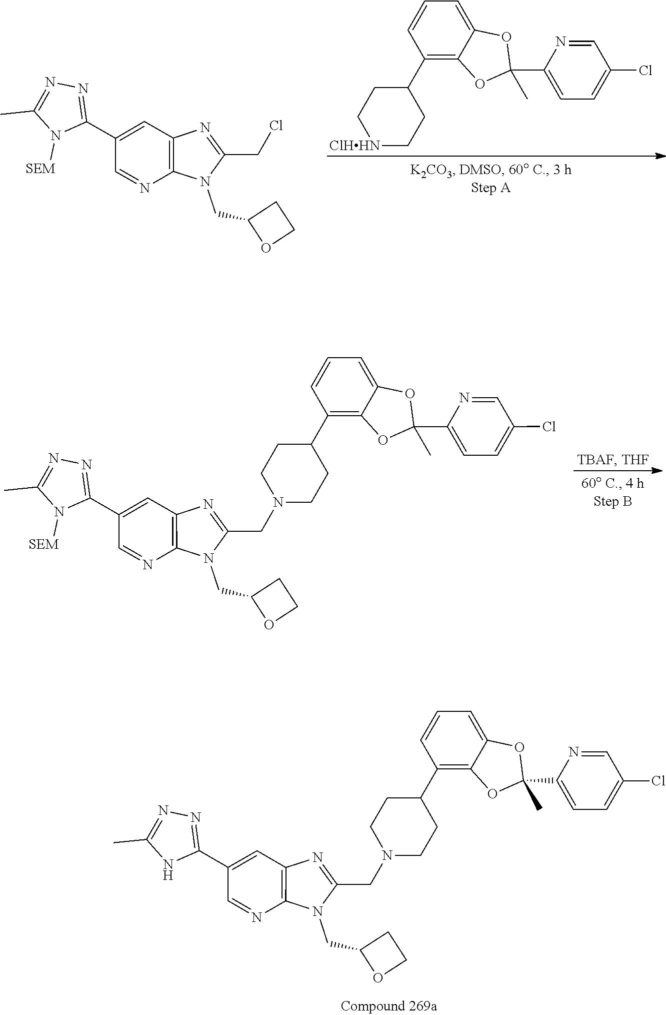

### Step A: 2-((4-(2-(5-chloropyridin-2-yl)-2-methylbenzo[d][1,3]dioxol-4-yl)piperidin-1-yl)methyl)-6-(5-methyl-4-((2-(trimethylsilyl)ethoxy)methyl)-4H-1,2,4-triazol-3-yl)-3-(((S)-oxetan-2-yl)methyl)-3H-imidazo[4,5-b]pyridine

A mixture of 5-chloro-2-(2-methyl-4-(piperidin-4-yl)benzo[d][1,3]dioxol-2-yl)pyridine hydrochloride (34 mg, 0.09 mmol), K₂CO₃ (37 mg, 0.27 mmol) and TEA (46 mg, 0.45 mmol) in DMSO (3 mL) was stirred at room temperature for 10 mins. (S)-2-(chloromethyl)-6-(5-methyl-4-((2-(trimethylsilyl)ethoxy)methyl)-4H-1,2,4-triazol-3-yl)-3-(oxetan-2-ylmethyl)-3H-imidazo[4,5-b]pyridine (40 mg, 0.09 mmol) was added and the reaction mixture was stirred at 60° C. for 3 hours. The mixture was poured into cold water (10 mL) and extracted with EtOAc (2×10 mL), the combined organic layers were washed with brine, dried over sodium sulfate, filtered and concentrated under reduced pressure. The residue was purified by prep-TLC to furnish 2-((4-(2-(5-chloropyridin-2-yl)-2-methylbenzo[d][1,3]dioxol-4-yl)piperidin-1-yl)methyl)-6-(5-methyl-4-((2-(trimethylsilyl)ethoxy)methyl)-4H-1,2,4-triazol-3-yl)-3-(((S)-oxetan-2-yl)methyl)-3H-imidazo[4,5-b]pyridine (23 mg, yield: 34%) as a yellow solid. MS Calcd.: 742.3; MS Found: 743.3 [M+H]⁺.

### Step B: 5-chloro-2-[2-methyl-4-(1-{[6-(5-methyl-4H-1,2,4-triazol-3-yl)-3-{[(2S)-oxetan-2-yl]methyl}-3H-imidazo[4,5-b]pyridin-2-yl]methyl}piperidin-4-yl)-2H-1,3-benzodioxol-2-yl]pyridine (Compound 269a)

A mixture of 2-((4-(2-(5-chloropyridin-2-yl)-2-methylbenzo[d][1,3]dioxol-4-yl)piperidin-1-yl)methyl)-6-(5-methyl-4-((2-(trimethylsilyl)ethoxy)methyl)-4H-1,2,4-triazol-3-yl)-3-(((S)-oxetan-2-yl)methyl)-3H-imidazo[4,5-b]pyridine (23 mg, 0.031 mmol) and TBAF/THF (1M, 5 mL) in THE (1 mL) was stirred at 60° C. for 4 hours. The reaction mixture was diluted with DCM and washed with water. The organic layer was concentrated and purified by prep-HPLC (0.1% of TFA) to give 5-chloro-2-[2-methyl-4-(1-{[6-(5-methyl-4H-1,2,4-triazol-3-yl)-3-f{[(2S)-oxetan-2-yl]methyl}-3H-imidazo[4,5-b]pyridin-2-yl]methyl}piperidin-4-yl)-2H-1,3-benzodioxol-2-yl]pyridine Compound 269a (1.6 mg, yield: 8.5%) as a yellow solid.

MS Calcd.: 612.2; MS Found: 613.2 [M+H]⁺.

¹H NMR (400 MHz, MeOD-d4): δ 9.10 (d, J=1.2 Hz, 1H), 8.69 (d, J=1.6 Hz, 1H), 8.62 (s, 1H), 7.90 (dd, J1=6.0 Hz, J2=8.4 Hz, 1H), 7.68 (d, J=8.4 Hz, 1H), 6.88-6.75 (m, 3H), 5.30-5.20 (m, 2H), 4.72-4.68 (m, 2H), 4.45-4.35 (m, 1H), 4.00-3.88 (m, 2H), 3.45-3.30 (m, 2H), 3.18-3.00 (m, 2H), 2.85-2.75 (m, 1H), 2.54 (s, 3H), 2.21-2.12 (m, 5H), 2.11-2.00 (m, 4H).

### Example 209: 3-fluoro-4-[(3-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1,2,3,6-tetrahydropyridin-4-yl}phenoxy)methyl]benzonitrile (Compound 317a)

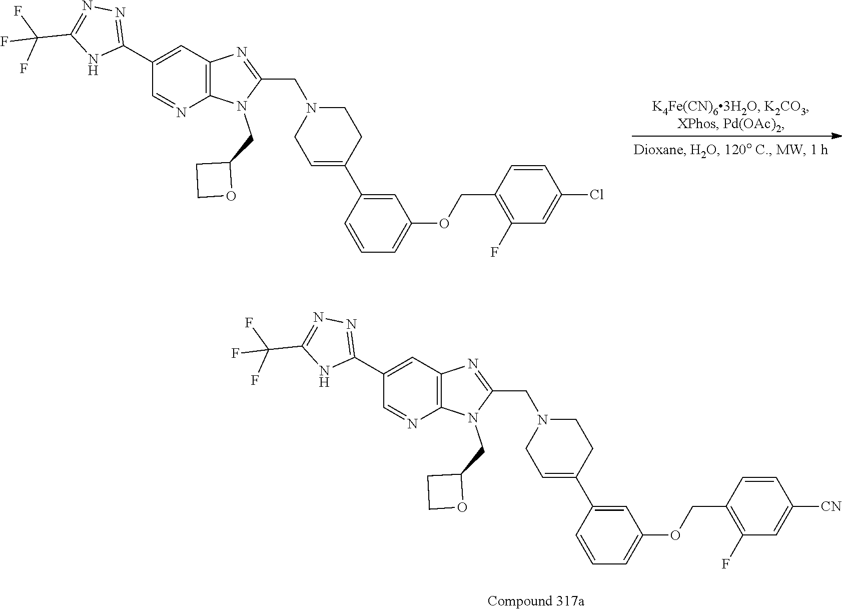 [SMILES: Fc1cc(Cl)ccc1COc1cccc(C2=CCN(Cc3nc4cc(-c5nnc(C(F)(F)F)[nH]5)cnc4n3C[C@@H]3CCO3)CC2)c1 ; N#Cc1ccc(COc2cccc(C3=CCN(Cc4nc5cc(-c6nnc(C(F)(F)F)[nH]6)cnc5n4C[C@@H]4CCO4)CC3)c2)c(F)c1]

A mixture of (S)-2-((4-(3-((4-chloro-2-fluorobenzyl)oxy)phenyl)-3,6-dihydropyridin-1(2H)-yl)methyl)-3-(oxetan-2-ylmethyl)-6-(5-(trifluoromethyl)-4H-1,2,4-triazol-3-yl)-3H-imidazo[4,5-b]pyridine (60 mg, 0.09 mmol), K₄Fe(CN)₆·3H₂O (39 mg, 0.09 mmol), X-Phos (9 mg, 0.02 mmol), Pd(OAc)₂ (92 mg, 0.01 mmol) and K₂CO₃ (38 mg, 0.27 mmol) in dioxane (2 mL) and H₂O (0.2 mL) was stirred at 120° C. in microwave for 1 hour. The mixture was filtration and purified by pre-HPLC to give 3-fluoro-4-[(3-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-1,2,3,6-tetrahydropyridin-4-yl}phenoxy)methyl]benzonitrile Compound 317a (6.8 mg, yield: 11%) as a white solid.

MS Calcd.: 644.2; MS Found: 645.2 [M+H]⁺.

¹H NMR (400 MHz, MeOD): δ 8.95 (m, 1H), 8.52 (m, 1H), 7.66-7.22 (t, J=7.6 Hz, 1H), 7.52-7.48 (m, 2H), 7.18-7.12 (t, J=8.2 Hz, 1H), 7.00-6.94 (m, 2H), 6.82-6.79 (m, 1H), 6.03 (br.s, 1H), 5.27-5.18 (m, 1H), 5.13 (s, 2H), 4.91-4.83 (m, 2H), 4.75-4.68 (m, 2H), 4.57-4.47 (m, 1H), 4.38-4.32 (m, 1H), 4.14-4.02 (m, 2H), 2.82-2.77 (m, 2H), 2.74-2.63 (m, 1H), 2.52-2.40 (m, 3H). ¹⁹F-NMR (377 MHz): −66.63, −117.46.

### Example 210: 3-fluoro-4-(2-methyl-4-{1-[(3-{[(2S)-oxetan-2-yl]methyl}-6-(5-oxo-4,5-dihydro-1,2,4-oxadiazol-3-yl)-3H-imidazo[4,5-b]pyridin-2-yl)methyl]piperidin-4-yl}-2H-1,3-benzodioxol-2-yl)benzonitrile (Compound 329a)

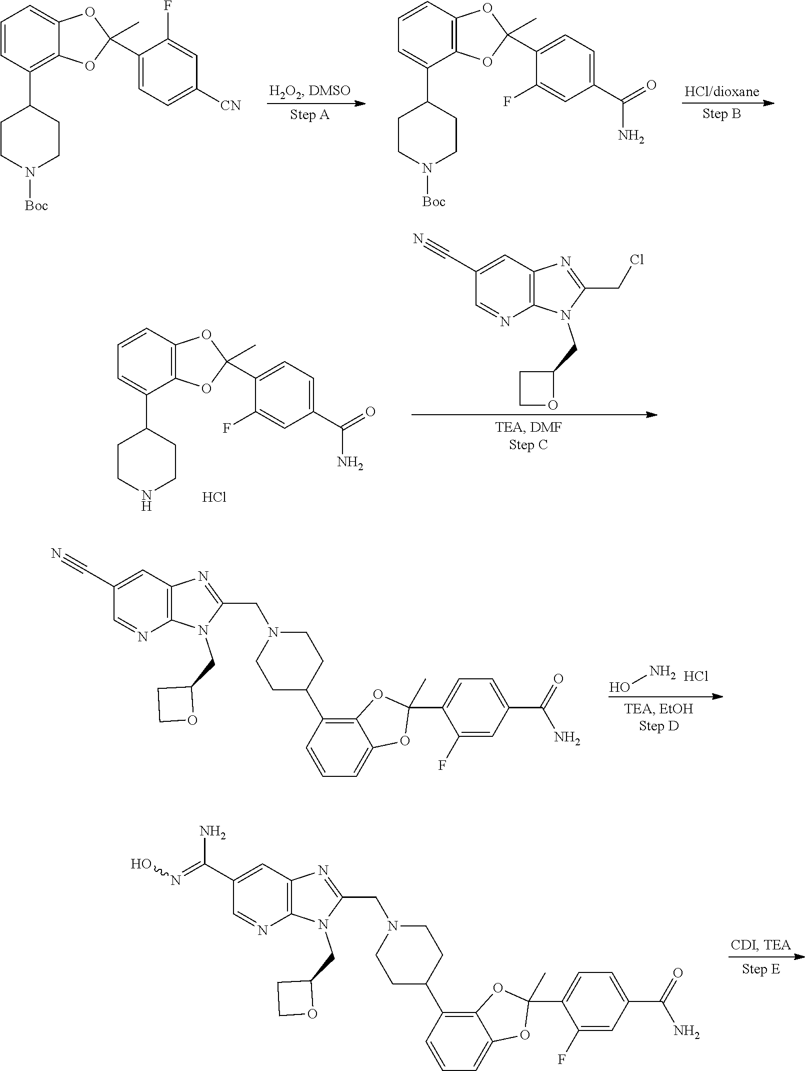

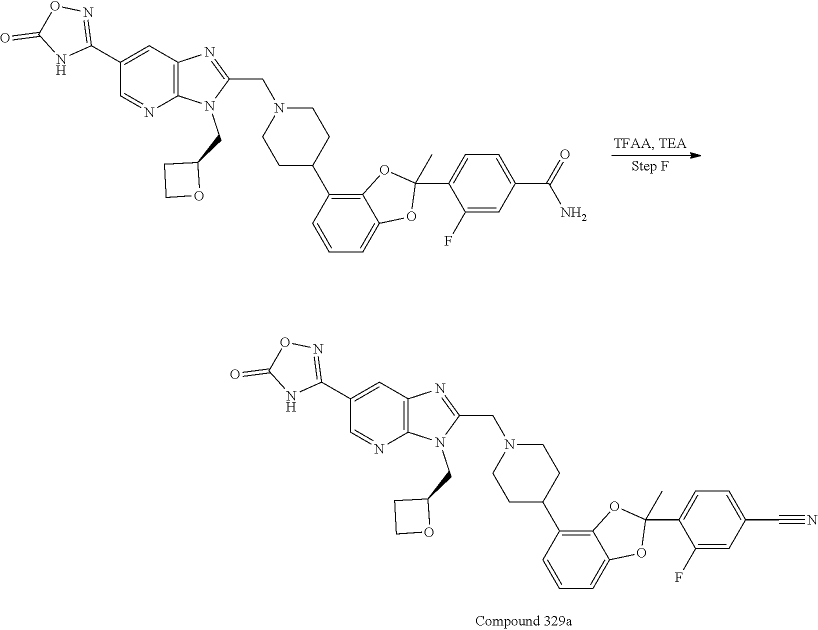 [SMILES: CC1(c2ccc(C(N)=O)cc2F)Oc2cccc(C3CCN(Cc4nc5cc(-c6noc(=O)[nH]6)cnc5n4C[C@@H]4CCO4)CC3)c2O1 ; CC1(c2ccc(C#N)cc2F)Oc2cccc(C3CCN(Cc4nc5cc(-c6noc(=O)[nH]6)cnc5n4C[C@@H]4CCO4)CC3)c2O1]

### Step A: tert-butyl 4-(2-(4-carbamoyl-2-fluorophenyl)-2-methylbenzo[d][1,3]dioxol-4-yl)piperidine-1-carboxylate

To a mixture of tert-butyl 4-(2-(4-cyano-2-fluorophenyl)-2-methylbenzo[d][1,3]dioxol-4-yl)piperidine-1-carboxylate (200 mg, 0.46 mmol) and K₂CO₃ (189 mg, 1.4 mmol) in DMSO (2 mL) was added H₂O₂ (0.5 ml). The reaction was stirred at room temperature for 3 hours. After the reaction was completed, the reaction was quenched with water (20 mL) and extracted with ethyl acetate (30 mL×3). The organic layer was combined and washed with brine (30 mL×2), dried over sodium sulfate, filtered and concentrated in vacuum. The residue was purified by column chromatography (PE:EA=5:1) to give tert-butyl 4-(2-(4-carbamoyl-2-fluorophenyl)-2-methylbenzo[d][1,3]dioxol-4-yl)piperidine-1-carboxylate (300 mg, ca. 100% yield) as a white solid. MS Calcd.: 456.2; MS Found: 479.6 [M+Na]⁺.

### Step B: 3-fluoro-4-(2-methyl-4-(piperidin-4-yl)benzo[d][1,3]dioxol-2-yl)benzamide HCl salt

To a solution of tert-butyl 4-(2-(4-carbamoyl-2-fluorophenyl)-2-methylbenzo[d][1,3]dioxol-4-yl)piperidine-1-carboxylate (300 mg, 0.66 mmol) in dioxane (3 mL) was added HCl/dioxane (3 mL, 4 N). The reaction was stirred at room temperature for 1 h. After the reaction was completed, the reaction was concentrated in vacuum to give 3-fluoro-4-(2-methyl-4-(piperidin-4-yl)benzo[d][1,3]dioxol-2-yl)benzamide HCl salt (400 mg, crude) as a yellow solid. MS Calcd.: 356.2; MS Found: 357.0 [M+H]⁺.

### Step C: 4-(4-(1-((6-cyano-3-(((S)-oxetan-2-yl)methyl)-3H-imidazo[4,5-b]pyridin-2-yl)methyl)piperidin-4-yl)-2-methylbenzo[d][1,3]dioxol-2-yl)-3-fluorobenzamide

A mixture of 3-fluoro-4-(2-methyl-4-(piperidin-4-yl)benzo[d][1,3]dioxol-2-yl)benzamide HCl salt (136 mg), (S)-2-(chloromethyl)-3-(oxetan-2-ylmethyl)-3H-imidazo[4,5-b]pyridine-6-carbonitrile (100 mg, 0.38 mmol) and TEA (116 mg, 1.15 mmol) in DMF (2 mL) was stirred at 50° C. for 2 hours. After the reaction was completed, the reaction was quenched with water (20 mL) and extracted with ethyl acetate (30 mL×3). The organic layer was combined and washed with brine (30 mL×2), dried over sodium sulfate, filtered and concentrated in vacuum. The residue was purified by column chromatography on silica gel (PE:EA=2:1) to give 4-(4-(1-((6-cyano-3-(((S)-oxetan-2-yl)methyl)-3H-imidazo[4,5-b]pyridin-2-yl)methyl)piperidin-4-yl)-2-methylbenzo[d][1,3]dioxol-2-yl)-3-fluorobenzamide (180 mg, 81% yield) as yellow solid. MS Calcd.: 582.2; MS Found: 583.1 [M+H]⁺.

### Step D: 3-fluoro-4-(4-(1-((6-(N′-hydroxycarbamimidoyl)-3-(((S)-oxetan-2-yl)methyl)-3H-imidazo[4,5-b]pyridin-2-yl)methyl)piperidin-4-yl)-2-methylbenzo[d][1,3]dioxol-2-yl)benzamide

To a solution of 4-(4-(1-((6-cyano-3-(((S)-oxetan-2-yl)methyl)-3H-imidazo[4,5-b]pyridin-2-yl)methyl)piperidin-4-yl)-2-methylbenzo[d][1,3]dioxol-2-yl)-3-fluorobenzamide (180 mg, 0.31 mmol) and TEA (93 mg, 0.93 mmol) in EtOH (3 mL) was added NH₂OH HCl (42 mg, 0.62 mmol). The reaction was stirred at 90° C. for 1 hours. After the reaction was completed, the reaction was quenched with water (20 mL) and extracted with ethyl acetate (50 mL×3). The organic layer was combined and washed with brine (50 mL×2), dried over sodium sulfate, filtered and concentrated in vacuum. The residue was purified by column chromatography (DCM:MeOH=10:1) to give 3-fluoro-4-(4-(1-((6-(N′-hydroxycarbamimidoyl)-3-(((S)-oxetan-2-yl)methyl)-3H-imidazo[4,5-b]pyridin-2-yl)methyl)piperidin-4-yl)-2-methylbenzo[d][1,3]dioxol-2-yl)benzamide (120 mg, 63% yield, crude) as a yellow solid. MS Calcd.: 615.3; MS Found: 616.5 [M+H]⁺.

### Step E: 3-fluoro-4-(2-methyl-4-(1-((3-(((S)-oxetan-2-yl)methyl)-6-(5-oxo-4,5-dihydro-1,2,4-oxadiazol-3-yl)-3H-imidazo[4,5-b]pyridin-2-yl)methyl)piperidin-4-yl)benzo[d][1,3]dioxol-2-yl)benzamide

A mixture of 3-fluoro-4-(4-(1-((6-(N′-hydroxycarbamimidoyl)-3-(((S)-oxetan-2-yl)methyl)-3H-imidazo[4,5-b]pyridin-2-yl)methyl)piperidin-4-yl)-2-methylbenzo[d][1,3]dioxol-2-yl)benzamide (120 mg, crude), CDI (24 mg, 0.15 mmol) and TEA (20 mg, 0.19 mmol) in dioxane (2 mL) was stirred at 80° C. for 2 hours. After the reaction was completed, the reaction was quenched with water (20 mL) and extracted with ethyl acetate (50 mL×3). The organic layer was combined and washed with brine (50 mL×2), dried over sodium sulfate, filtered and concentrated in vacuum. The residue was purified by column chromatography on silica gel (DCM:MeOH=10:1) to give 3-fluoro-4-(2-methyl-4-(1-((3-(((S)-oxetan-2-yl)methyl)-6-(5-oxo-4,5-dihydro-1,2,4-oxadiazol-3-yl)-3H-imidazo[4,5-b]pyridin-2-yl)methyl)piperidin-4-yl)benzo[d][1,3]dioxol-2-yl)benzamide (150 mg, crude) as a yellow solid. MS Calcd.: 641.2; MS Found: 642.5 [M+H]⁺.

### Step F: 3-fluoro-4-(2-methyl-4-{1-[(3-{[(2S)-oxetan-2-yl]methyl}-6-(5-oxo-4,5-dihydro-1,2,4-oxadiazol-3-yl)-3H-imidazo[4,5-b]pyridin-2-yl)methyl]piperidin-4-yl}-2H-1,3-benzodioxol-2-yl)benzonitrile (Compound 329a)

To a solution of 3-fluoro-4-(2-methyl-4-(1-((3-(((S)-oxetan-2-yl)methyl)-6-(5-oxo-4,5-dihydro-1,2,4-oxadiazol-3-yl)-3H-imidazo[4,5-b]pyridin-2-yl)methyl)piperidin-4-yl)benzo[d][1,3]dioxol-2-yl)benzamide (150 mg, crude) and TEA (147 mg, 1.45 mmol) in DCM (5 mL) was added TFAA (255 mg, 1.21 mmol). The reaction was stirred at room temperature for 2 hours. After the reaction was completed, the reaction was concentrated and the residue purified by Prep-HPLC to give 3-fluoro-4-(2-methyl-4-{1-[(3-{[(2S)-oxetan-2-yl]methyl}-6-(5-oxo-4,5-dihydro-1,2,4-oxadiazol-3-yl)-3H-imidazo[4,5-b]pyridin-2-yl)methyl]piperidin-4-yl}-2H-1,3-benzodioxol-2-yl)benzonitrile Compound 329a (5.0 mg, 4% yield) as a white solid.

MS Calcd.: 623.2; MS Found: 624.2 [M+H]⁺.

¹H NMR (400 MHz, DMSO-d₆): δ 8.88 (dd, J=2.0 Hz, J=0.8 Hz, 1H), 8.54 (d, J=2.0 Hz, 1H), 7.81 (t, J=8.0, 1H), 7.64-7.70 (m, 1H), 7.59 (dd, J=8.0 Hz, J=1.2 Hz, 1H), 6.78-6.90 (m, 3H), 5.21-5.30 (m, 1H), 4.65-4.80 (m, 3H), 4.37-4.45 (m, 1H), 3.88-4.05 (m, 2H), 3.35-3.50 (m, 2H), 3.05-3.15 (m, 1H), 2.75-2.85 (m, 1H), 2.48-2.57 (m, 1H), 2.13-2.30 (m, 4H), 2.09 (s, 3H). (Some peaks partially overlapped with solvent). ¹⁹F-NMR (377 MHz): −111.98.

### Example 211: 4-[(2S)-2-(4-chloro-2-fluorophenyl)-2-methyl-2H-1,3-benzodioxol-4-yl]-1-[(3-{[(2S)-oxetan-2-yl]methyl}-6-(1H-1,2,3,4-tetrazol-5-yl)-3H-imidazo[4,5-c]pyridin-2-yl)methyl]piperidine (Compound 417a)

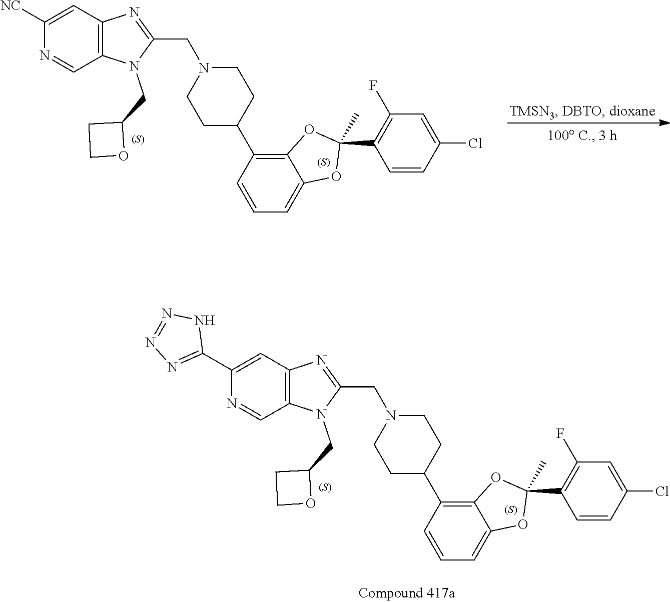

A mixture of 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (50 mg, 0.087 mmol), TMSN₃ (30 mg, 0.261 mmol) and DBTO (43 mg, 0.17 mmol) in dioxane (1.5 mL) was heated to 100° C. for 3 hours under an atmosphere of nitrogen. The solvent was removed in vacuo. The crude was purified by Prep-HPLC to afford 4-[(2S)-2-(4-chloro-2-fluorophenyl)-2-methyl-2H-1,3-benzodioxol-4-yl]-1-[(3-{[(2S)-oxetan-2-yl]methyl}-6-(1H-1,2,3,4-tetrazol-5-yl)-3H-imidazo[4,5-c]pyridin-2-yl)methyl]piperidine Compound 417a (22 mg, yield: 42%) as a white solid.

MS Calcd.: 616.2; MS Found: 617.1 [M+H]⁺.

¹H NMR (400 MHz, DMSO-d₆): δ 9.20 (s, 1H), 8.38 (s, 1H), 7.52-7.60 (m, 2H), 7.34 (dd, J=8.4 Hz, 1.6 Hz, 1H), 6.72-6.81 (m, 3H), 5.15-5.22 (m, 1H), 4.87-4.96 (m, 1H), 4.73-4.79 (m, 1H), 4.47-4.54 (m, 1H), 4.38-4.45 (m, 1H), 4.02 (d, J=13.6 Hz, 1H), 3.87 (d, J=14.0 Hz, 1H), 3.03-3.08 (m, 1H), 2.87-2.93 (m, 1H), 2.62-2.78 (m, 2 H), 2.43-2.51 (m, 1H), 2.17-2.35 (m, 2H), 2.03 (s, 3H), 1.69-1.82 (m, 4H). ¹⁹F-NMR (377 MHz): −110.78.

### Example 212: 4-[(2S)-2-(4-chloro-2-fluorophenyl)-2-methyl-2H-1,3-benzodioxol-4-yl]-1-({3-[(2R)-2-methoxypropyl]-6-[5-(trifluoromethyl)-4H-1,2,4-triazol-3-yl]-3H-imidazo[4,5-c]pyridin-2-yl}methyl)piperidine (Compound 418a)

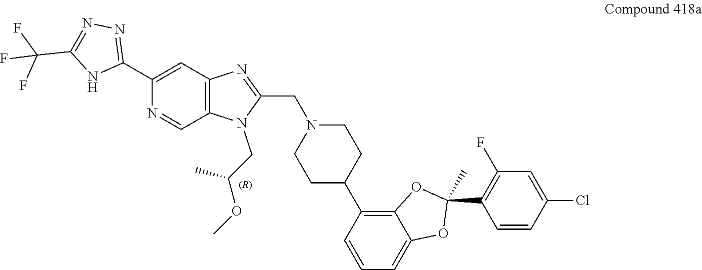

Starting from (R)-2-methoxypropan-1-amine hydrochloride, 4-[(2S)-2-(4-chloro-2-fluorophenyl)-2-methyl-2H-1,3-benzodioxol-4-yl]-1-({3-[(2R)-2-methoxypropyl]-6-[5-(trifluoromethyl)-4H-1,2,4-triazol-3-yl]-3H-imidazo[4,5-c]pyridin-2-yl}methyl)piperidine Compound 418a (37 mg) was obtained with similar procedure of Compound 345a.

MS Calcd.: 685.2; MS Found: 686.2 [M+H]⁺.

¹H NMR (400 MHz, DMSO-d₆): δ 9.07 (s, 1H), 8.26 (s, 1H), 7.52-7.59 (m, 2H), 7.32 (dd, J=8.0 Hz, 1.6 Hz, 1H), 6.78-6.81 (m, 2H), 6.71-6.77 (m, 1H), 4.45-4.60 (m, 2H), 4.06 (d, J=14.0 Hz, 1H), 3.85-3.95 (m, 1H), 3.79 (d, J=13.6 Hz, 1H), 3.02-3.11 (m, 4H), 2.82-2.90 (m, 1H), 2.60-2.70 (m, 1H), 2.25-2.35 (m, 1H), 2.15-2.24 (m, 1H), 2.02 (s, 3H), 1.70-1.84 (m, 4H), 1.22 (d, J=6.4 Hz, 3H). ¹⁹F-NMR (377 MHz): −63.41, −110.65.

### Example 213: 3-[2-({4-[(2S)-2-(4-chloro-2-fluorophenyl)-2-methyl-2H-1,3-benzodioxol-4-yl]piperidin-1-yl}methyl)-3-[(2R)-2-methoxypropyl]-3H-imidazo[4,5-c]pyridin-6-yl]-4,5-dihydro-1,2,4-oxadiazol-5-one (Compound 419a)

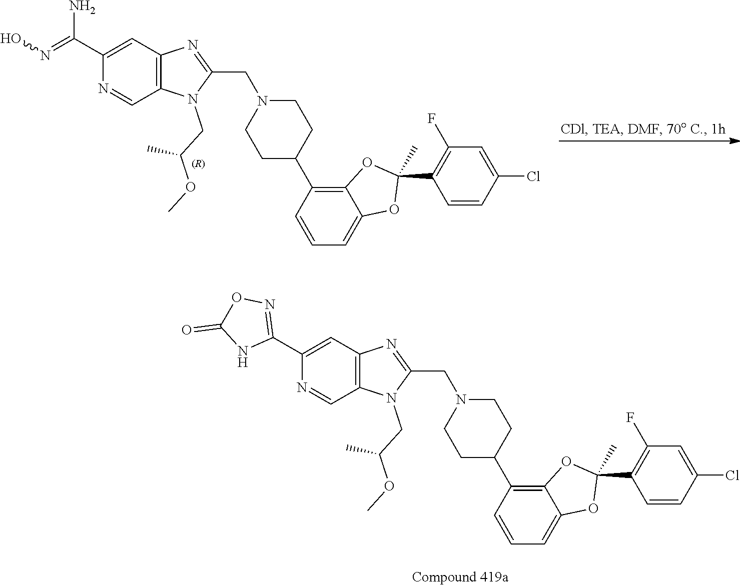

To a solution of 2-((4-((S)-2-(4-chloro-2-fluorophenyl)-2-methylbenzo[d][1,3]dioxol-4-yl)piperidin-1-yl)methyl)-N′-hydroxy-3-((R)-2-methoxypropyl)-3H-imidazo[4,5-c]pyridine-6-carboximidamide (60 mg) in DMF (1.5 mL) was added TEA (30 mg, 0.30 mmol) and CDI (24 mg, 0.15 mmol). The resulting mixture was stirred at 70° C. for 1 hour. The solvent was removed in vacuo. The residue was purified by Prep-HPLC to give 3-[2-({4-[(2S)-2-(4-chloro-2-fluorophenyl)-2-methyl-2H-1,3-benzodioxol-4-yl]piperidin-1-yl}methyl)-3-[(2R)-2-methoxypropyl]-3H-imidazo[4,5-c]pyridin-6-yl]-4,5-dihydro-1,2,4-oxadiazol-5-one Compound 419a (31 mg, yield: 51%) as a white solid.

MS Calcd.: 634.2; MS Found: 635.2 [M+H]⁺.

¹H NMR (400 MHz, DMSO-d₆): δ 9.05 (s, 1H), 8.14 (s, 1H), 7.51-7.59 (m, 2H), 7.32 (d, J=8.0 Hz, 1H), 6.78-6.81 (m, 2H), 6.70-6.76 (m, 1H), 4.43-4.58 (m, 2H), 4.05 (d, J=13.6 Hz, 1H), 3.83-3.94 (m, 1H), 3.78 (d, J=13.6 Hz, 1H), 3.02-3.10 (m, 4H), 2.82-2.88 (m, 1H), 2.59-2.70 (m, 1H), 2.25-2.35 (m, 1H), 2.16-2.24 (m, 1H), 2.02 (s, 3H), 1.70-1.82 (m, 4H), 1.21 (d, J=6.0 Hz, 3H). ¹⁹F-NMR (377 MHz): −110.65.

### Example 214: 4-[(2S)-2-(4-chloro-2-fluorophenyl)-2-methyl-2H-1,3-benzodioxol-4-yl]-1-({3-[(2S)-2-methoxypropyl]-6-[5-(trifluoromethyl)-4H-1,2,4-triazol-3-yl]-3H-imidazo[4,5-c]pyridin-2-yl}methyl)piperidine (Compound 420a)

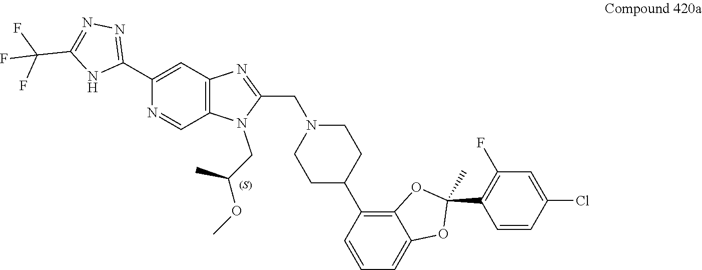

Starting from (S)-2-methoxypropan-1-amine HCl salt, 4-[(2S)-2-(4-chloro-2-fluorophenyl)-2-methyl-2H-1,3-benzodioxol-4-yl]-1-({3-[(2S)-2-methoxypropyl]-6-[5-(trifluoromethyl)-4H-1,2,4-triazol-3-yl]-3H-imidazo[4,5-c]pyridin-2-yl}methyl)piperidine Compound 420a (34 mg) was obtained with similar procedure of Compound 345a as a white solid.

MS Calcd.: 685.2; MS Found: 686.3 [M+H]⁺.

¹H NMR (400 MHz, DMSO-d₆): δ 9.08 (s, 1H), 8.27 (s, 1H), 7.53-7.60 (m, 2H), 7.32-7.36 (m, 1H), 6.70-6.81 (m, 3H), 4.45-4.60 (m, 2H), 4.08 (d, J=13.6 Hz, 1H), 3.87-3.98 (m, 1H), 3.79 (d, J=14.4 Hz, 1H), 3.04-3.12 (m, 4H), 2.81-2.88 (m, 1H), 2.60-2.70 (m, 1H), 2.15-2.35 (m, 2H), 2.02 (s, 3H), 1.68-1.90 (m, 4H), 1.22 (d, J=6.0 Hz, 3H). ¹⁹F-NMR (377 MHz): −63.56, −110.69.

### Example 215: 3-[2-({4-[(2S)-2-(4-chloro-2-fluorophenyl)-2-methyl-2H-1,3-benzodioxol-4-yl]piperidin-1-yl}methyl)-3-[(2S)-2-methoxypropyl]-3H-imidazo[4,5-c]pyridin-6-yl]-4,5-dihydro-1,2,4-oxadiazol-5-one (Compound 421a)

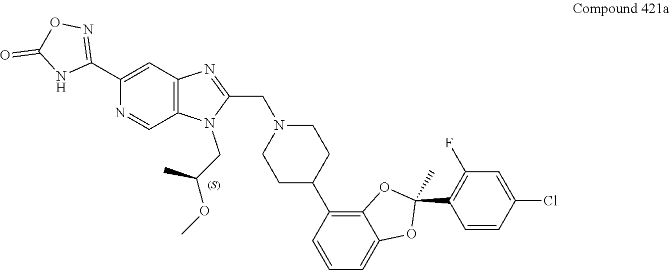

3-[2-({4-[(2S)-2-(4-chloro-2-fluorophenyl)-2-methyl-2H-1,3-benzodioxol-4-yl]piperidin-1-yl}methyl)-3-[(2S)-2-methoxypropyl]-3H-imidazo[4,5-c]pyridin-6-yl]-4,5-dihydro-1,2,4-oxadiazol-5-one Compound 421a (11 mg) was obtained as a white solid.

MS Calcd.: 634.2; MS Found: 635.3 [M+H]⁺.

¹H NMR (400 MHz, DMSO-d₆): δ 9.06 (s, 1H), 8.15 (s, 1H), 7.53-7.60 (m, 2H), 7.34 (dd, J=8.4 Hz, 1.6 Hz, 1H), 6.70-6.82 (m, 3H), 4.45-4.58 (m, 2H), 4.07 (d, J=14.0 Hz, 1H), 3.87-3.95 (m, 1H), 3.78 (d, J=14.0 Hz, 1H), 3.02-3.11 (m, 4H), 2.80-2.87 (m, 1H), 2.58-2.70 (m, 1H), 2.15-2.33 (m, 2H), 2.01 (s, 3H), 1.67-1.90 (m, 4H), 1.21 (d, J=6.0 Hz, 3H). ¹⁹F-NMR (377 MHz): −110.69.

### Example 216: 3-[2-({4-[(2R)-2-(4-chlorophenyl)-2,3-dihydro-1,4-benzodioxin-5-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one (Compound 422a)

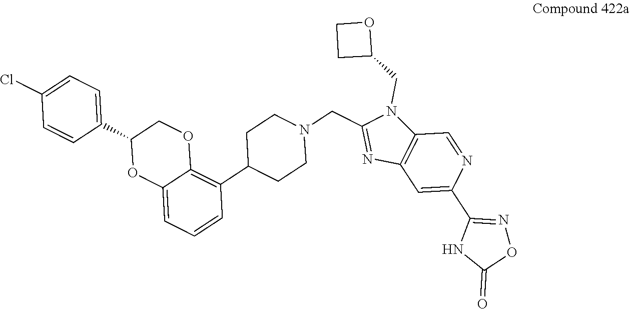 [SMILES: O=c1[nH]c(-c2cc3nc(CN4CCC(c5cccc6c5OC[C@@H](c5ccc(Cl)cc5)O6)CC4)n(C[C@@H]4CCO4)c3cn2)no1]

Starting from (R)-4-(2-(4-chlorophenyl)-2,3-dihydrobenzo[b][1,4]dioxin-5-yl)piperidine, 3-[2-({4-[(2R)-2-(4-chlorophenyl)-2,3-dihydro-1,4-benzodioxin-5-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one Compound 422a (17 mg) was obtained as a white solid.

MS Calcd.: 614.2; MS Found: 615.1 [M+H]⁺.

¹H NMR (400 MHz, DMSO-d₆): δ 9.30 (s, 1H), 8.29 (s, 1H), 7.51 (s, 4H), 6.88-6.93 (m, 2H), 6.77-6.85 (m, 1H), 5.27-5.30 (m, 1H), 5.07-5.15 (m, 1H), 4.80-5.00 (m, 3H), 4.73-4.80 (m, 1H), 4.47-4.56 (m, 2H), 4.37-4.45 (m, 1H), 4.08-4.17 (m, 1H), 3.65-3.80 (m, 2H), 3.21-3.40 (m, 2H), 3.10-3.22 (m, 1H), 2.70-2.80 (m, 1H), 2.30-2.41 (m, 1H), 1.88-2.10 (m, 4H).

### Example 217: 4-[(2R)-2-(4-chlorophenyl)-2,3-dihydro-1,4-benzodioxin-5-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 423a)

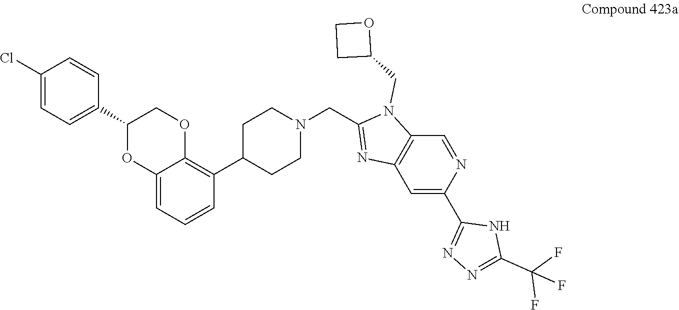 [SMILES: FC(F)(F)c1nnc(-c2cc3nc(CN4CCC(c5cccc6c5OC[C@@H](c5ccc(Cl)cc5)O6)CC4)n(C[C@@H]4CCO4)c3cn2)[nH]1]

4-[(2R)-2-(4-chlorophenyl)-2,3-dihydro-1,4-benzodioxin-5-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine Compound 423a (18 mg) was obtained as a white solid.

MS Calcd.: 665.2; MS Found: 666.1 [M+H]⁺.

¹H NMR (400 MHz, DMSO-d₆): δ 9.18 (s, 1H), 8.30 (s, 1H), 7.47-7.53 (m, 4H), 6.7-6.85 (m, 3H), 5.22-5.29 (m, 1H), 5.15-5.21 (m, 1H), 4.90-4.99 (m, 1H), 4.75-4.81 (m, 1H), 4.40-4.58 (m, 2H), 4.39-4.58 (m, 1H), 3.80-4.10 (m, 3H), 2.97-3.12 (m, 1H), 2.81-2.98 (m, 2H), 2.70-2.81 (m, 1H), 2.40-2.51 (m, 1H), 2.11-2.40 (m, 2H), 1.58-1.83 (m, 4H).

### Example 218: 4-[(2R)-2-(4-chlorophenyl)-2,3-dihydro-1,4-benzodioxin-5-yl]-1-{[6-(5-methyl-4H-1,2,4-triazol-3-yl)-3-{[(2S)-oxetan-2-yl]methyl}-3H-imidazo[4,5-c]pyridin-2-yl]methyl}piperidine (Compound 424a)

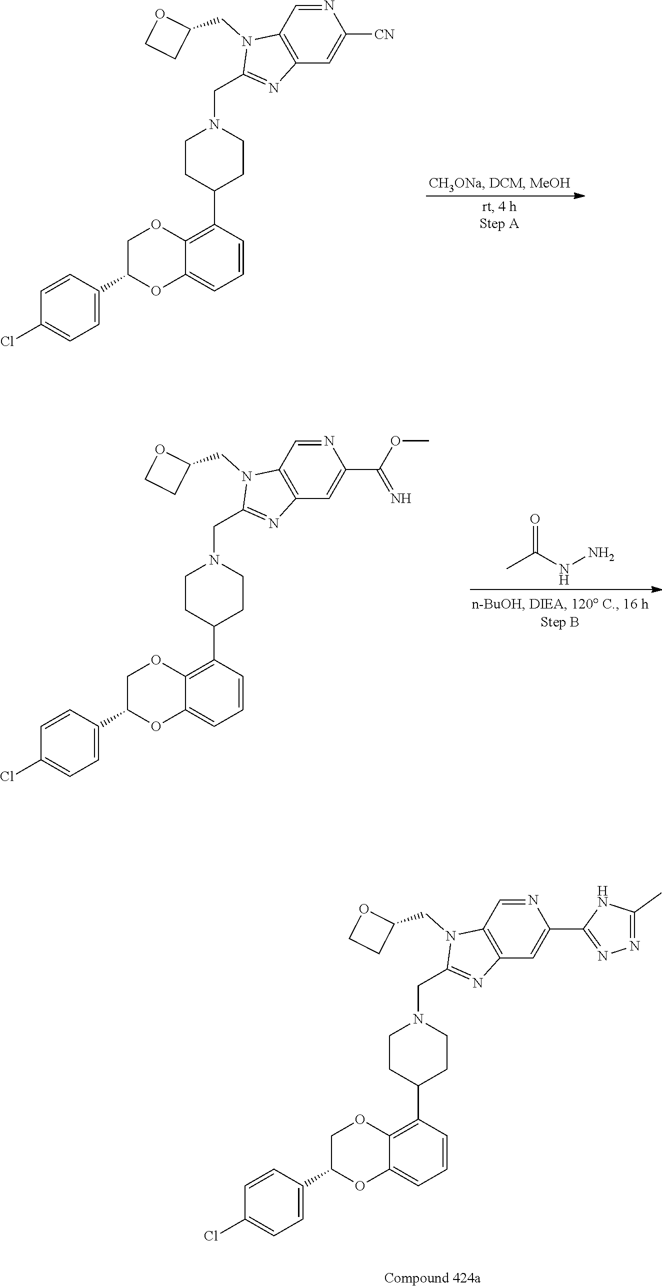

### Step A: methyl 2-((4-((R)-2-(4-chlorophenyl)-2,3-dihydrobenzo[b][1,4]dioxin-5-yl)piperidin-1-yl)methyl)-3-(((S)-oxetan-2-yl)methyl)-3H-imidazo[4,5-c]pyridine-6-carbimidate

To a solution of 2-((4-((R)-2-(4-chlorophenyl)-2,3-dihydrobenzo[b][1,4]dioxin-5-yl)piperidin-1-yl)methyl)-3-(((S)-oxetan-2-yl)methyl)-3H-imidazo[4,5-c]pyridine-6-carbonitrile (80 mg, 0.144 mmol) in DCM (2 mL) and MeOH (2 mL) was added sodium methanolate (78 mg, 1.44 mmol). The solution was stirred at room temperature for 4 hours. The reaction was quenched with water (10 mL), extracted with ethyl acetate (10 mL×2). The organic layers were combined, dried over Na₂SO₄, filtered, and concentrated under vacuum. The residue was purified by Prep-TLC (DCM/MeOH=12/1) to give methyl 2-((4-((R)-2-(4-chlorophenyl)-2,3-dihydrobenzo[b][1,4]dioxin-5-yl)piperidin-1-yl)methyl)-3-(((S)-oxetan-2-yl)methyl)-3H-imidazo[4,5-c]pyridine-6-carbimidate (30 mg, yield: 35%) as a white solid. MS Calcd.: 587.2; MS Found: 588.2 [M+H]⁺.

### Step B: 4-[(2R)-2-(4-chlorophenyl)-2,3-dihydro-1,4-benzodioxin-5-yl]-1-{[6-(5-methyl-4H-1,2,4-triazol-3-yl)-3-{[(2S)-oxetan-2-yl]methyl}-3H-imidazo[4,5-c]pyridin-2-yl]methyl}piperidine (Compound 424a)

A mixture of methyl 2-((4-((R)-2-(4-chlorophenyl)-2,3-dihydrobenzo[b][1,4]dioxin-5-yl)piperidin-1-yl)methyl)-3-(((S)-oxetan-2-yl)methyl)-3H-imidazo[4,5-c]pyridine-6-carbimidate (30 mg, 0.051 mmol) in n-BuOH (2 mL) was added acetohydrazide (7.5 mg, 0.10 mmol) and DIEA (19.7 mg, 0.15 mmol). The solution was stirred at 120° C. for 16 hours. The solvent was removed in vacuo. The residue was purified by Prep-HPLC to give 4-[(2R)-2-(4-chlorophenyl)-2,3-dihydro-1,4-benzodioxin-5-yl]-1-{[6-(5-methyl-4H-1,2,4-triazol-3-yl)-3-t{[(2S)-oxetan-2-yl]methyl}-3H-imidazo[4,5-c]pyridin-2-yl]methyl}piperidine Compound 424a (9.7 mg, yield: 31%) as a white solid.

MS Calcd.: 611.2; MS Found: 612.3 [M+H]⁺.

¹H NMR (400 MHz, DMSO-d₆): δ 9.24 (s, 1H), 8.32 (s, 1H), 7.51 (s, 4H), 6.88-6.92 (m, 2H), 6.77-6.84 (m, 1H), 5.27-5.31 (m, 1H), 5.07-5.14 (m, 1H), 4.87-4.95 (m, 2H), 4.72-4.79 (m, 1H), 4.48-4.56 (m, 2H), 4.38-4.45 (m, 1H), 4.08-4.15 (m, 2H), 3.73-3.79 (m, 2H), 3.25-3.45 (m, 2H), 3.12-3.24 (m, 1H), 2.70-2.81 (m, 1H), 2.32-2.43 (m, 4H), 1.90-2.12 (m, 4H).

### Example 219: 5-chloro-2-(2-methyl-4-{1-[(3-{[(2S)-oxolan-2-yl]methyl}-6-[5-(trifluoromethyl)-4H-1,2,4-triazol-3-yl]-3H-imidazo[4,5-c]pyridin-2-yl)methyl]piperidin-4-yl}-2H-1,3-benzodioxol-2-yl)pyridine (Compound 425a)

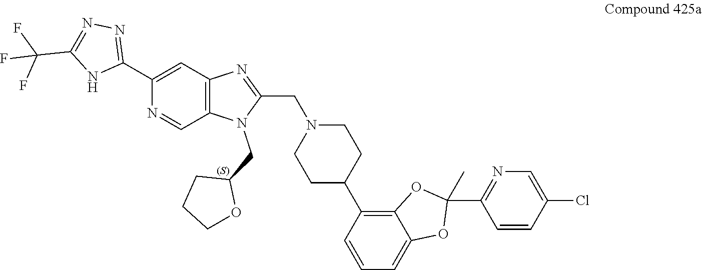

Starting from (S)-(tetrahydrofuran-2-yl)methanamine, 5-chloro-2-(2-methyl-4-{1-[(3-{[(2S)-oxolan-2-yl]methyl}-6-[5-(trifluoromethyl)-4H-1,2,4-triazol-3-yl]-3H-imidazo[4,5-c]pyridin-2-yl)methyl]piperidin-4-yl}-2H-1,3-benzodioxol-2-yl)pyridine Compound 425a (9.3 mg) was obtained with similar procedure of Compound 345a as a white solid.

MS Calcd.: 680.2; MS Found: 681.4 [M+H]⁺.

¹H NMR (400 MHz, DMSO-d₆): δ 9.10 (s, 1H), 8.72 (t, J=2.8 Hz, 1H), 8.27 (s, 1H), 7.98-8.03 (m, 1H), 7.57-7.62 (m, 1H), 6.72-6.81 (m, 3H), 4.60-4.67 (m, 1H), 4.48-4.57 (m, 1H), 4.27-4.37 (m, 1H), 4.00-4.08 (m, 1H), 3.75-3.88 (m, 2H), 3.57-3.68 (m, 1H), 3.00-3.10 (m, 1H), 2.80-2.87 (m, 1H), 2.57-2.70 (m, 1H), 2.25-2.35 (m, 1H), 2.14-2.25 (m, 1H), 2.04-2.13 (m, 1H), 2.00 (d, J=2.8 Hz, 3H), 1.63-1.93 (m, 7H). ¹⁹F-NMR (377 MHz): −63.73.

### Example 220: 4-[(2S)-2-(4-chloro-2-fluorophenyl)-2-methyl-2H-1,3-benzodioxol-4-yl]-1-[(3-{[(2S)-oxolan-2-yl]methyl}-6-[5-(trifluoromethyl)-4H-1,2,4-triazol-3-yl]-3H-imidazo[4,5-c]pyridin-2-yl)methyl]piperidine (Compound 426a)

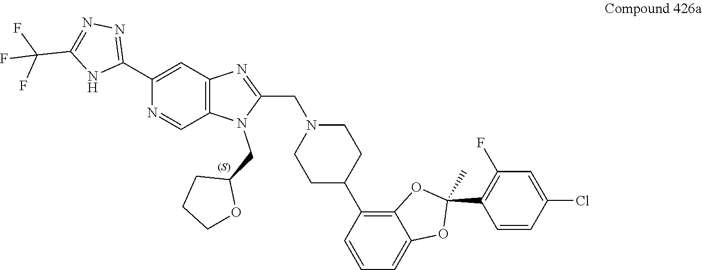

4-[(2S)-2-(4-chloro-2-fluorophenyl)-2-methyl-2H-1,3-benzodioxol-4-yl]-1-[(3-{[(2S)-oxolan-2-yl]methyl}-6-[5-(trifluoromethyl)-4H-1,2,4-triazol-3-yl]-3H-imidazo[4,5-c]pyridin-2-yl)methyl]piperidine Compound 426a (25 mg) was obtained.

MS Calcd.: 697.2; MS Found: 698.3 [M+H]⁺.

¹H NMR (400 MHz, DMSO-d₆): δ 9.11 (s, 1H), 8.28 (s, 1H), 7.52-7.60 (m, 2H), 7.33 (dd, J=8.4, 2.0 Hz, 1H), 6.69-6.81 (m, 3H), 4.62-4.70 (m, 1H), 4.50-4.60 (m, 1H), 4.29-4.38 (m, 1H), 4.06 (d, J=13.6 Hz, 1H), 3.77-3.90 (m, 2H), 3.60-3.70 (m, 1H), 3.03-3.10 (m, 1H), 2.80-2.89 (m, 1H), 2.60-2.70 (m, 1H), 2.25-2.35 (m, 1H), 2.15-2.25 (m, 1H), 2.05-2.15 (m, 1H), 2.02 (s, 3H), 1.85-1.96 (m, 1H), 1.75-1.85 (m, 3H), 1.62-1.75 (m, 3H). ¹⁹F-NMR (377 MHz): −63.73, −110.75.

### Example 221: 3-fluoro-4-[(2S)-2-methyl-4-(1-{[6-(5-methyl-4H-1,2,4-triazol-3-yl)-3-{[(2S)-oxetan-2-yl]methyl}-3H-imidazo[4,5-c]pyridin-2-yl]methyl}piperidin-4-yl)-2H-1,3-benzodioxol-2-yl]benzonitrile (Compound 288a)

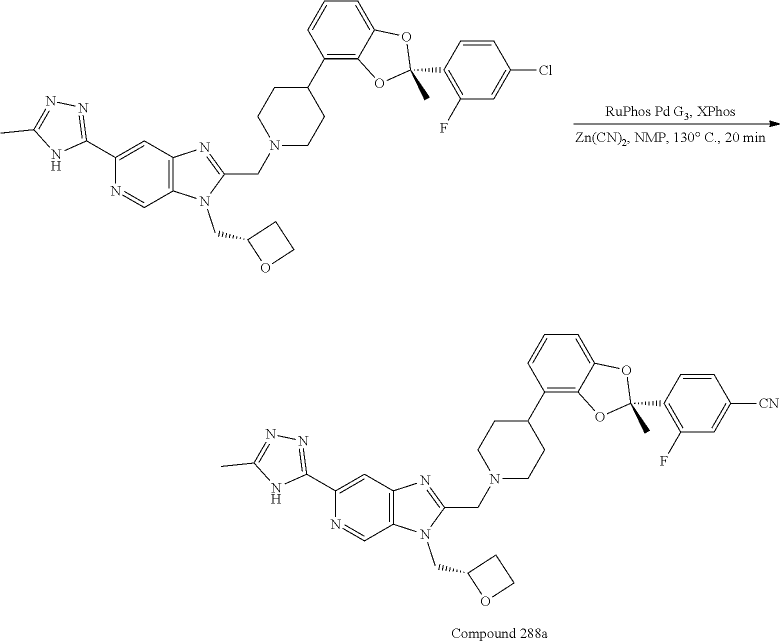 [SMILES: Cc1nnc(-c2cc3nc(CN4CCC(c5cccc6c5O[C@@](C)(c5ccc(Cl)cc5F)O6)CC4)n(C[C@@H]4CCO4)c3cn2)[nH]1 ; Cc1nnc(-c2cc3nc(CN4CCC(c5cccc6c5O[C@@](C)(c5ccc(C#N)cc5F)O6)CC4)n(C[C@@H]4CCO4)c3cn2)[nH]1]

A mixture of 2-((4-((S)-2-(4-chloro-2-fluorophenyl)-2-methylbenzo[d][1,3]dioxol-4-yl)piperidin-1-yl)methyl)-6-(5-methyl-4H-1,2,4-triazol-3-yl)-3-(((S)-oxetan-2-yl)methyl)-3H-imidazo[4,5-c]pyridine (10 mg, 0.016 mmol), Zn(CN)₂ (0.56 mg, 0.048 mmol), RuPhos Pd G3 (1.3 mg, 0.0016 mmol) and XPhos (0.76 mg, 0.0016 mmol) in NMP (1 mL) was stirred at 130° C. for 20 min. The reaction mixture was filtered and the filtrate purified by Prep-HPLC to give 3-fluoro-4-[(2S)-2-methyl-4-(1-{[6-(5-methyl-4H-1,2,4-triazol-3-yl)-3-{[(2S)-oxetan-2-yl]methyl}-3H-imidazo[4,5-c]pyridin-2-yl]methyl}piperidin-4-yl)-2H-1,3-benzodioxol-2-yl]benzonitrile Compound 288a (2.6 mg, 25% yield) as a white solid.

MS Calcd.: 620.3; MS Found: 621.5 [M+H]⁺.

¹H NMR (400 MHz, DMSO-d₆): 9.14 (s, 1H), 8.48 (s, 1H), 7.75-7.85 (m, 1H), 7.53-7.73 (m, 2H), 6.70-6.90 (m, 3H), 5.15-5.30 (m, 2H), 4.63-4.80 (m, 4H), 4.40-4.55 (m, 1H), 3.85-4.18 (m, 2H), 3.44-3.51 (m, 3H), 2.54 (s, 3H), 2.09-2.40 (m, 9H). ¹⁹F-NMR (377 MHz): −111.95.

### Example 222: 3-fluoro-4-(2-methyl-4-{1-[(3-{[(2S)-oxetan-2-yl]methyl}-6-(5-oxo-4,5-dihydro-1,2,4-oxadiazol-3-yl)-3H-imidazo[4,5-c]pyridin-2-yl)methyl]piperidin-4-yl}-2H-1,3-benzodioxol-2-yl)benzonitrile (Compound 341a)

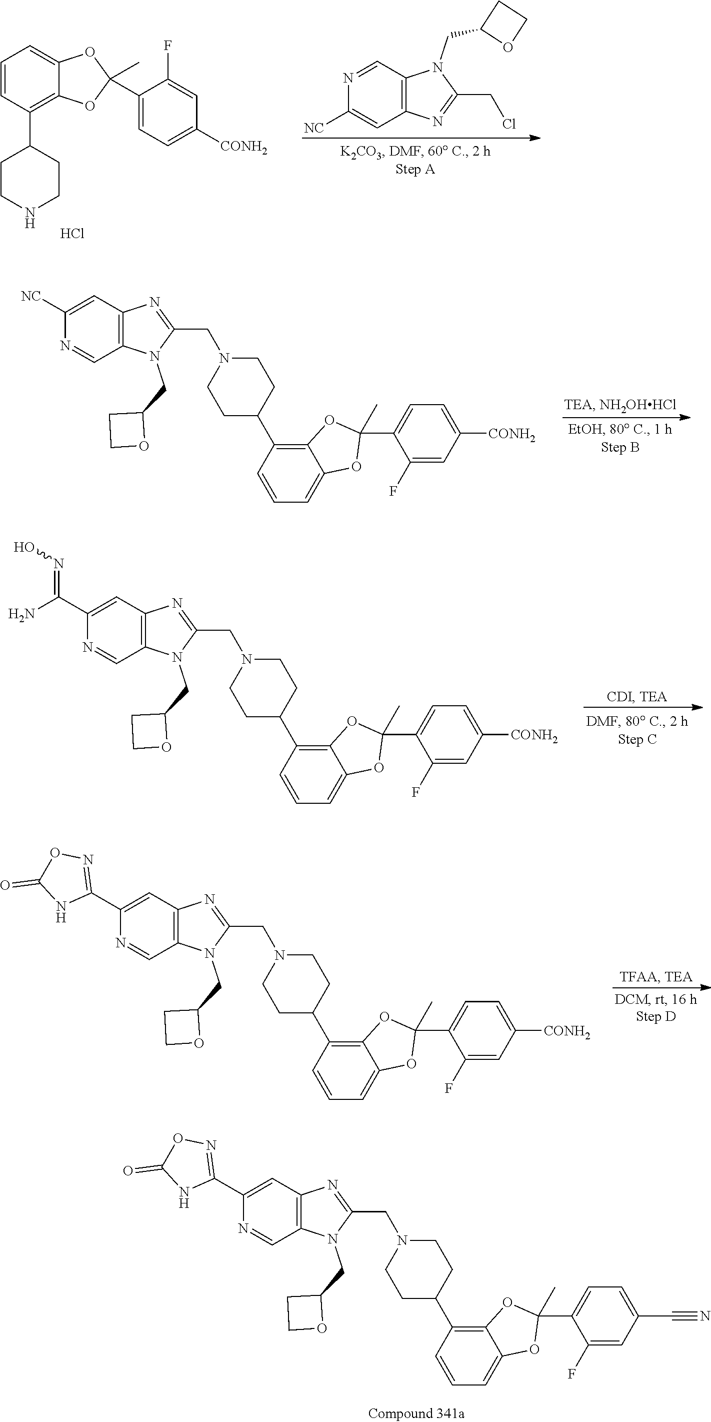

### Step A: 4-(4-(1-((6-cyano-3-(((S)-oxetan-2-yl)methyl)-3H-imidazo[4,5-c]pyridin-2-yl)methyl)piperidin-4-yl)-2-methylbenzo[d][1,3]dioxol-2-yl)-3-fluorobenzamide

A mixture of 3-fluoro-4-(2-methyl-4-(piperidin-4-yl)benzo[d][1,3]dioxol-2-yl)benzamide HCl salt (50 mg), (S)-2-(chloromethyl)-3-(oxetan-2-ylmethyl)-3H-imidazo[4,5-c]pyridine-6-carbonitrile (30 mg, 0.11 mmol) and K₂CO₃ (53 mg, 0.38 mmol) in DMF (2 mL) was stirred at 60° C. for 2 hours. After the reaction was completed, the mixture was filtered, and the filtrate concentrated in vacuum. The residue was purified by Prep-TLC (DCM:MeOH=30:1) to give 4-(4-(1-((6-cyano-3-(((S)-oxetan-2-yl)methyl)-3H-imidazo[4,5-c]pyridin-2-yl)methyl)piperidin-4-yl)-2-methylbenzo[d][1,3] dioxol-2-yl)-3-fluorobenzamide (28 mg, 42% yield) as a white solid. MS Calcd.: 582.2; MS Found: 583.2 [M+H]⁺.

### Step B: 3-fluoro-4-(4-(1-((6-(N′-hydroxycarbamimidoyl)-3-(((S)-oxetan-2-yl)methyl)-3H-imidazo[4,5-c]pyridin-2-yl)methyl)piperidin-4-yl)-2-methylbenzo[d][1,3]dioxol-2-yl)benzamide

To a solution of 4-(4-(1-((6-cyano-3-(((S)-oxetan-2-yl)methyl)-3H-imidazo[4,5-c]pyridin-2-yl)methyl)piperidin-4-yl)-2-methylbenzo[d][1,3]dioxol-2-yl)-3-fluorobenzamide (28 mg, 0.048 mmol) and TEA (19 mg, 0.19 mmol) in EtOH (2 mL) was added NH₂OH HCl (6.7 mg, 0.096 mmol). The reaction was stirred at 80° C. for 1 hour. After the reaction was completed, the reaction was quenched with water (5 mL) and extracted with ethyl acetate (10 mL×3). The organic layer was dried over sodium sulfate, filtered and concentrated to give crude 3-fluoro-4-(4-(1-((6-(N′-hydroxycarbamimidoyl)-3-(((S)-oxetan-2-yl)methyl)-3H-imidazo[4,5-c]pyridin-2-yl)methyl)piperidin-4-yl)-2-methylbenzo[d][1,3]dioxol-2-yl)benzamide (40 mg, 100% yield) as a white solid. MS Calcd.: 615.3; MS Found: 616.3 [M+H]⁺.

### Step C: 3-fluoro-4-(2-methyl-4-(1-((3-(((S)-oxetan-2-yl)methyl)-6-(5-oxo-4,5-dihydro-1,2,4-oxadiazol-3-yl)-3H-imidazo[4,5-c]pyridin-2-yl)methyl)piperidin-4-yl)benzo[d][1,3]dioxol-2-yl)benzamide

A mixture of 3-fluoro-4-(4-(1-((6-(N′-hydroxycarbamimidoyl)-3-(((S)-oxetan-2-yl)methyl)-3H-imidazo[4,5-c]pyridin-2-yl)methyl)piperidin-4-yl)-2-methylbenzo[d][1,3]dioxol-2-yl)benzamide (40 mg), CDI (16 mg, 0.098 mmol) and TEA (20 mg, 0.19 mmol) in DMF (2 mL) was stirred at 80° C. for 2 hours. After the reaction was completed, the reaction was quenched with water (10 mL) and extracted with ethyl acetate (15 mL×3). The organic layer was combined and washed with brine (10 mL×2), dried over sodium sulfate, filtered and concentrated to give crude 3-fluoro-4-(2-methyl-4-(1-((3-(((S)-oxetan-2-yl)methyl)-6-(5-oxo-4,5-dihydro-1,2,4-oxadiazol-3-yl)-3H-imidazo[4,5-c]pyridin-2-yl)methyl)piperidin-4-yl)benzo[d][1,3]dioxol-2-yl)benzamide (42 mg) as yellow oil. MS Calcd.: 641.2; MS Found: 642.1 [M+H]⁺.

### Step D: 3-fluoro-4-(2-methyl-4-{1-[(3-{[(2S)-oxetan-2-yl]methyl}-6-(5-oxo-4,5-dihydro-1,2,4-oxadiazol-3-yl)-3H-imidazo[4,5-c]pyridin-2-yl)methyl]piperidin-4-yl}-2H-1,3-benzodioxol-2-yl)benzonitrile (Compound 341a)

To a solution of 3-fluoro-4-(2-methyl-4-(1-((3-(((S)-oxetan-2-yl)methyl)-6-(5-oxo-4,5-dihydro-1,2,4-oxadiazol-3-yl)-3H-imidazo[4,5-c]pyridin-2-yl)methyl)piperidin-4-yl)benzo[d][1,3]dioxol-2-yl)benzamide (42 mg) and TEA (40 mg, 0.39 mmol) in DCM (3.0 mL) was added TFAA (41 mg, 0.19 mmol). The reaction was stirred at room temperature for 16 hours. After the reaction was completed, the reaction was quenched with water (5 mL) and extracted with (DCM/MeOH=30:1) (10 mL×3). The organic layer was dried over sodium sulfate, filtered and concentrated in vacuum. The residue was purified by Prep-HPLC (0.1% TFA/H₂O/CH₃CN) to give 3-fluoro-4-(2-methyl-4-{1-[(3-{[(2S)-oxetan-2-yl]methyl}-6-(5-oxo-4,5-dihydro-1,2,4-oxadiazol-3-yl)-3H-imidazo[4,5-c]pyridin-2-yl)methyl]piperidin-4-yl}-2H-1,3-benzodioxol-2-yl)benzonitrile Compound 341a (5.4 mg, 13% yield) as a white solid.

MS Calcd.: 623.2; MS Found: 624.2 [M+H]⁺.

¹H NMR (400 MHz, DMSO-d₆): δ 9.28 (s, 1H), 8.27 (s, 1H), 8.00 (d, J=10.8 Hz, 1H), 7.74-7.80 (m, 2H), 6.80-6.90 (m, 2H), 6.70-6.80 (m, 1H), 5.07-5.15 (m, 1H), 4.88-4.97 (m, 1H), 4.72-4.81 (m, 1H), 4.47-4.55 (m, 1H), 4.37-4.45 (m, 1H), 3.50-3.85 (m, 2H), 2.65-3.11 (m, 4H), 2.32-2.45 (m, 2H), 1.85-2.15 (m, 8H). ¹⁹F-NMR (377 MHz): −110.86.

### Example 223: 3-fluoro-4-[(2S)-2-methyl-4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}-2H-1,3-benzodioxol-2-yl]benzonitrile (Compound 342a)

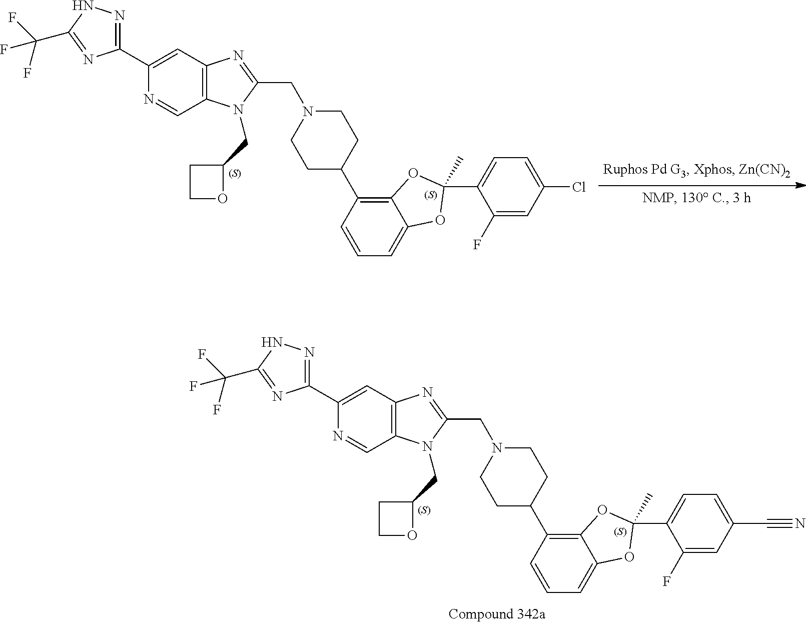

A mixture of 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-6-(5-(trifluoromethyl)-1H-1,2,4-triazol-3-yl)-3H-imidazo[4,5-c]pyridine (30 mg, 0.044 mmol), Zn(CN)₂ (15.5 mg, 0.13 mmol), RuPhos Pd G3 (7.4 mg, 0.009 mmol) and X-Phos (6.3 mg, 0.013 mmol) in anhydrous NMP (2.0 mL) was stirred at 130° C. for 3 h under Ar. The reaction mixture was filtered and the filtrate purified by Prep-HPLC (0.1% FA/H₂O/CH₃CN) to give 3-fluoro-4-[(2S)-2-methyl-4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}-2H-1,3-benzodioxol-2-yl]benzonitrile Compound 342a (15 mg, yield: 50%) as white solid.

MS Calcd.: 674.2; MS Found: 675.3 [M+H]⁺.

¹H NMR (400 MHz, DMSO-d6): δ 9.17 (s, 1H), 8.29 (s, 1H), 7.98 (d, J=10.4 Hz, 1H), 7.70-7.78 (m, 2H), 6.74-6.82 (m, 3H), 5.14-5.22 (m, 1H), 4.88-4.97 (m, 1H), 4.73-4.80 (m, 1H), 4.48-4.55 (m, 1H), 4.40-4.47 (m, 1H), 4.02 (d, J=13.6 Hz, 1H), 3.87 (d, J=13.6 Hz, 1H), 3.01-3.08 (m, 1H), 2.85-2.92 (m, 1H), 2.60-2.80 (m, 3H), 2.15-2.35 (m, 2H), 2.05 (s, 3H), 1.68-1.82 (m, 4H). ¹⁹F-NMR (377 MHz): −110.86.

### Example 224: 4-[(2S)-2-(4-chloro-2-fluorophenyl)-2-methyl-2H-1,3-benzodioxol-4-yl]-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 359a)

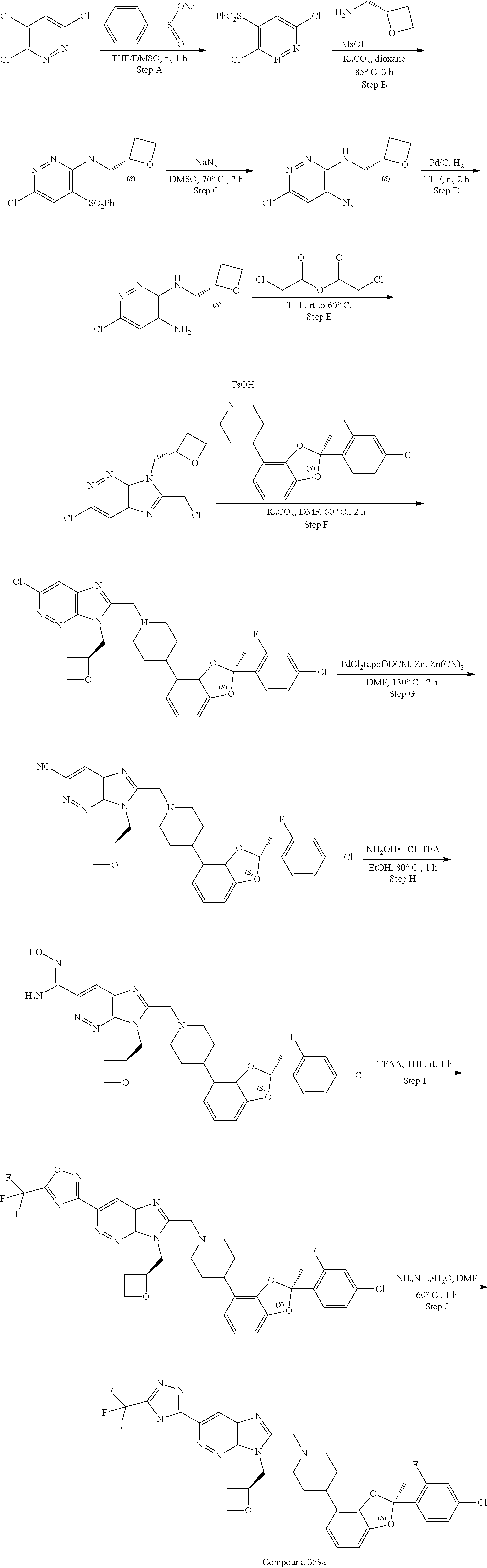

### Step A: 3,6-dichloro-4-(phenylsulfonyl)pyridazine

To a solution of 3,4,6-trichloropyridazine (15.0 g, 81.8 mmol) in THF/DMSO (5:1) (120 mL) was added sodium benzenesulfinate (18.0 g, 90.0 mmol). The reaction mixture was stirred at room temperature for 1 hour. After the reaction was completed, the mixture was quenched with H₂O (100 mL) and extracted with EA (200 mL*2). The combined organic layers were dried over anhydrous sodium sulfate, filtered and concentrated to dryness. The residue was purified by column chromatography on silica gel (PE/EA=10/1) to give 3,6-dichloro-4-(phenylsulfonyl)pyridazine (16.0 g, yield: 68%) as a white solid. MS Calcd.: 288.0; MS Found: 289.0 [M+H]⁺.

### Step B: (S)-6-chloro-N-(oxetan-2-ylmethyl)-4-(phenylsulfonyl)pyridazin-3-amine

A mixture of 3,6-dichloro-4-(phenylsulfonyl)pyridazine (5.0 g, 17.4 mmol), (S)-oxetan-2-ylmethanamine. MsOH salt (3.5 g, 19.1 mmol), K₂CO₃ (9.6 g, 69.4 mmol) in dioxane (100 mL) was stirred at 85° C. for 3 hours. After the reaction was completed, the reaction mixture was filtered, and the filtrate concentrated to dryness. The residue was purified by column chromatography on silica gel (PE/EA=3/1) to give (S)-6-chloro-N-(oxetan-2-ylmethyl)-4-(phenylsulfonyl)pyridazin-3-amine (4.3 g, yield: 73%) as yellow solid. MS Calcd.: 339.0; MS Found: 340.0 [M+H]⁺.

### Step C: (S)-4-azido-6-chloro-N-(oxetan-2-ylmethyl)pyridazin-3-amine

To a mixture of (S)-6-chloro-N-(oxetan-2-ylmethyl)-4-(phenylsulfonyl)pyridazin-3-amine (4.3 g, 12.7 mmol) in DMSO (70 mL) was added NaN₃ (3.3 g, 50.7 mmol) and the mixture stirred at 70° C. for 2 hours. After the reaction was completed, the mixture was quenched with H₂O (100 mL) and extracted with EA (200 mL*2). The extracts were wash with brine (200 mL*3). The organic layer was dried over anhydrous sodium sulfate, filtered, and concentrated to dryness. The residue was purified by column chromatography on silica gel (PE/EA=1/1) to give (S)-4-azido-6-chloro-N-(oxetan-2-ylmethyl)pyridazin-3-amine (2.3 g, yield: 74%) as brown oil. MS Calcd.: 240.0; MS Found: 241.2 [M+H]⁺.

### Step D: (S)-6-chloro-N3-(oxetan-2-ylmethyl)pyridazine-3,4-diamine

To a solution of (S)-4-azido-6-chloro-N-(oxetan-2-ylmethyl)pyridazin-3-amine (2.2 g, 10.3 mmol) in THE (40 mL) was added Pd/C (440 mg, 10% on Carbon, wetted with ca. 55% water). The mixture was stirred at rt for 2 hours under H₂ (1 atm). The reaction mixture was filtered through a celite pad. The filtrate was concentrated to give (S)-6-chloro-N3-(oxetan-2-ylmethyl)pyridazine-3,4-diamine (2.0 g crude) as a brown solid. MS Calcd.: 214.1; MS Found: 215.1 [M+H]⁺.

### Step E: (S)-3-chloro-6-(chloromethyl)-7-(oxetan-2-ylmethyl)-7H-imidazo[4,5-c]pyridazine

To a mixture of (S)-6-chloro-N3-(oxetan-2-ylmethyl)pyridazine-3,4-diamine (2.0 g) in THE (50 mL) was added 2-chloroacetic anhydride (1.6 g, 9.3 mmol). The mixture was stirred at rt for 1 h and then heated to 60° C. for 7 hours. The reaction mixture was concentrated, and the residue purified by column chromatography on silica gel (PE/EA=1/1) to give (S)-3-chloro-6-(chloromethyl)-7-(oxetan-2-ylmethyl)-7H-imidazo[4,5-c]pyridazine (1.1 g, yield: 43%) as yellow oil. MS Calcd.: 272.0; MS Found: 272.9 [M+H]⁺.

### Step F: 3-chloro-6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine

A mixture of (S)-4-(2-(4-chloro-2-fluorophenyl)-2-methylbenzo[d][1,3]dioxol-4-yl)piperidine. TsOH salt (1.2 g, 2.2 mmol), (S)-3-chloro-6-(chloromethyl)-7-(oxetan-2-ylmethyl)-7H-imidazo[4,5-c]pyridazine (600 mg, 2.2 mmol), K₂CO₃ (911 mg, 6.6 mmol) in DMF (10 mL) was stirred at 60° C. for 2 hours. After the reaction was completed, the mixture was diluted with EA (60 mL), washed with H₂O (30 mL*2). The combined organic layers were dried over anhydrous sodium sulfate, filtered and concentrated to dryness. The residue was purified by column chromatography on silica gel (PE/EA=2/1) to give 3-chloro-6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine (820 mg, yield: 63%) as light-yellow oil. MS Calcd.: 583.2; MS Found: 584.0 [M+H]⁺.

### Step G: 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carbonitrile

A mixture of 3-chloro-6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine (200 mg, 0.34 mmol), Zn(CN)₂ (40.3 mg, 0.34 mmol), Pd(dppf)Cl₂·DCM (55.5 mg, 0.068 mmol) and Zn (44.2 mg, 0.68 mmol) in DMF (2.0 mL) was stirred at 130° C. for 2 hours under N₂. The residue was purified directly by Prep-HPLC (0.1% TFA/H₂O/CH₃CN) to give 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carbonitrile (15 mg, yield: 7.6%) as a yellow solid. MS Calcd.: 574.2; MS Found: 575.1 [M+H]⁺.

### Step H: 6-((4-((S)-2-(4-chloro-2-fluorophenyl)-2-methylbenzo[d][1,3]dioxol-4-yl)piperidin-1-yl)methyl)-N′-hydroxy-7-(((S)-oxetan-2-yl)methyl)-7H-imidazo[4,5-c]pyridazine-3-carboximidamide

A solution of 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carbonitrile (15 mg, 0.026 mmol), NH₂OH HCl (4 mg, 0.052 mmol), TEA (11 mg, 0.10 mmol) in EtOH (2 mL) was stirred at 80° C. for 1 h. After the reaction was completed, the mixture was quenched with H₂O (5 mL) and extracted with DCM/MeOH (20:1) (15 mL*3). The combined organic layers were dried over anhydrous sodium sulfate, filtered and concentrated to give 6-((4-((S)-2-(4-chloro-2-fluorophenyl)-2-methylbenzo[d][1,3]dioxol-4-yl)piperidin-1-yl)methyl)-N′-hydroxy-7-(((S)-oxetan-2-yl)methyl)-7H-imidazo[4,5-c]pyridazine-3-carboximidamide (20 mg, crude) as light yellow solid. MS Calcd.: 607.2; MS Found: 608.1 [M+H]⁺.

### Step I: 3-(6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazin-3-yl)-5-(trifluoromethyl)-1,2,4-oxadiazole

To a solution of 6-((4-((S)-2-(4-chloro-2-fluorophenyl)-2-methylbenzo[d][1,3]dioxol-4-yl)piperidin-1-yl)methyl)-N′-hydroxy-7-(((S)-oxetan-2-yl)methyl)-7H-imidazo[4,5-c]pyridazine-3-carboximidamide (20 mg) in THE (2.0 mL) was added TFAA (21 mg, 0.099 mmol) and the mixture was stirred at rt for 1 hour. After the reaction was completed, the reaction was quenched with sat. sodium bicarbonate aqueous solution (5.0 mL), extracted with ethyl acetate (15 mL×3). The organic layers were combined, dried over Na₂SO₄, filtered and concentrated under vacuum to dryness. The residue was purified by Prep-TLC (PE/EA=2/1) to give 3-(6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazin-3-yl)-5-(trifluoromethyl)-1,2,4-oxadiazole (6 mg, yield: 26%) as a yellow solid. MS Calcd.: 685.2; MS Found: 686.2 [M+H]⁺.

### Step J: 4-[(2S)-2-(4-chloro-2-fluorophenyl)-2-methyl-2H-1,3-benzodioxol-4-yl]-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 359a)

A mixture of 3-(6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazin-3-yl)-5-(trifluoromethyl)-1,2,4-oxadiazole (6 mg, 0.009 mmol) and N₂H₄·H₂O (2 mg, 0.026 mmol) in DMF (0.5 mL) was stirred at 60° C. for 1 hour. After the reaction was completed, the mixture was purified directly by Prep-HPLC (0.1% TFA/H₂O/CH₃CN) to give 4-[(2S)-2-(4-chloro-2-fluorophenyl)-2-methyl-2H-1,3-benzodioxol-4-yl]-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine Compound 359a (1.1 mg, yield: 16%).

MS Calcd.: 684.2; MS Found: 685.2 [M+H]⁺.

¹H NMR (400 MHz, DMSO-d₆): δ 8.65 (s, 1H), 7.54-7.64 (m, 2H), 7.34 (dd, J=8.8 Hz, 2.0 Hz, 1H), 6.73-6.88 (m, 3H), 5.19-5.27 (m, 1H), 4.95-5.05 (m, 1H), 4.82-4.90 (m, 1H), 4.40-4.57 (m, 1H), 4.32-4.39 (m, 1H), 3.50-3.90 (m, 2H), 2.83-3.10 (m, 3H), 2.66-2.82 (m, 2H), 2.31-2.40 (m, 1H), 1.90-2.20 (m, 8H). ¹⁹F-NMR (377 MHz): −63.66, −110.52.

### Example 225: 3-fluoro-4-(2-methyl-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl)benzonitrile (Compound 273a)

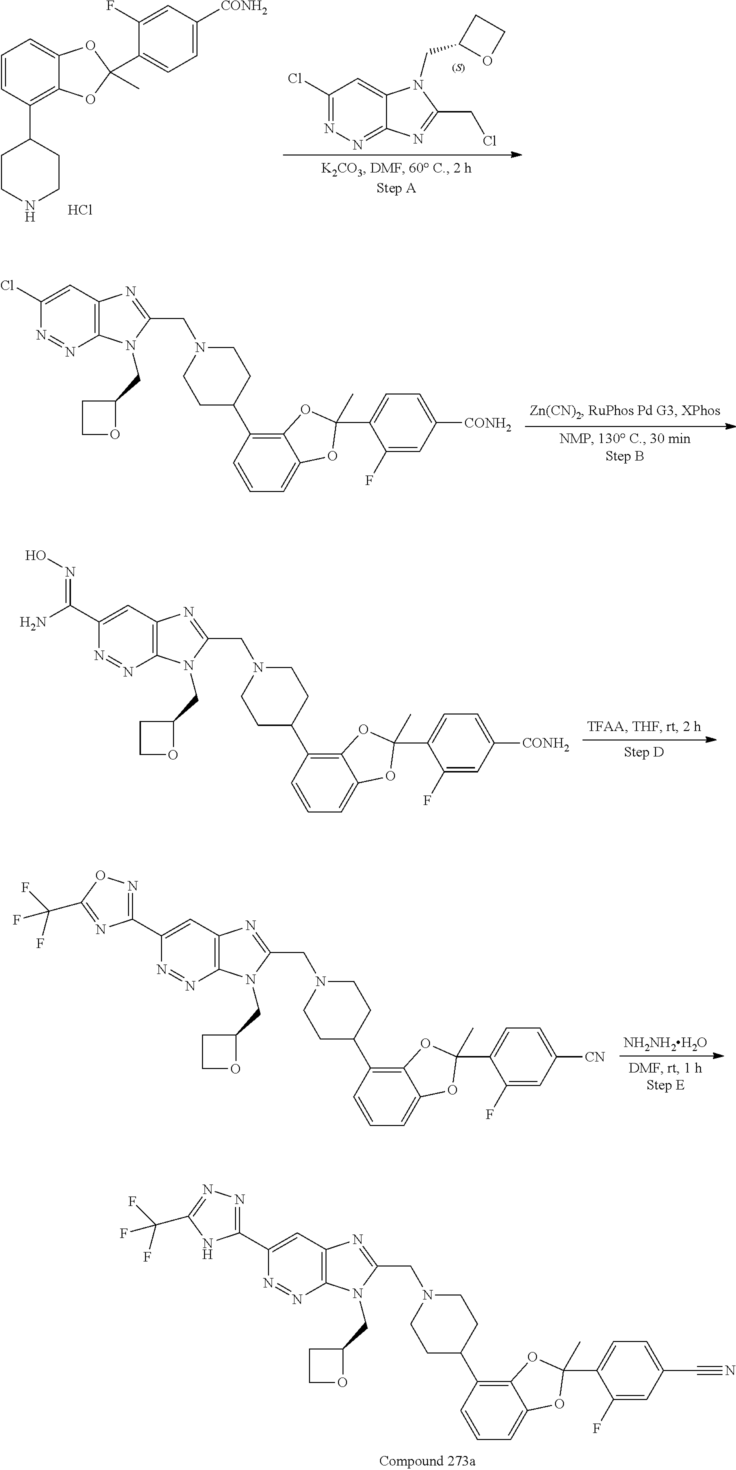

### Step A: 4-(4-(1-((3-chloro-7-(((S)-oxetan-2-yl)methyl)-7H-imidazo[4,5-c]pyridazin-6-yl)methyl)piperidin-4-yl)-2-methylbenzo[d][1,3]dioxol-2-yl)-3-fluorobenzamide

A mixture of 3-fluoro-4-(2-methyl-4-(piperidin-4-yl)benzo[d][1,3]dioxol-2-yl)benzamide HCl salt (395 mg), (S)-3-chloro-6-(chloromethyl)-5-(oxetan-2-ylmethyl)-5H-imidazo[4,5-c]pyridazine (200 mg, 0.74 mmol) and K₂CO₃ (304 mg, 2.21 mmol) in DMF (5 mL) was stirred at 60° C. for 2 hours. After the reaction was completed, the mixture was filtered, and the filtrate was concentrated in vacuum. The residue was purified by column chromatography on silica gel (DCM/MeOH=30/1) to give 4-(4-(1-((3-chloro-7-(((S)-oxetan-2-yl)methyl)-7H-imidazo[4,5-c]pyridazin-6-yl)methyl)piperidin-4-yl)-2-methylbenzo[d][1,3]dioxol-2-yl)-3-fluorobenzamide (140 mg, yield: 33%) as a yellow solid. MS Calcd.: 592.2; MS Found: 593.2 [M+H]⁺.

### Step B: 4-(4-(1-((3-cyano-7-(((S)-oxetan-2-yl)methyl)-7H-imidazo[4,5-c]pyridazin-6-yl)methyl)piperidin-4-yl)-2-methylbenzo[d][1,3]dioxol-2-yl)-3-fluorobenzamide

A mixture of 4-(4-(1-((3-chloro-7-(((S)-oxetan-2-yl)methyl)-7H-imidazo[4,5-c]pyridazin-6-yl)methyl)piperidin-4-yl)-2-methylbenzo[d][1,3]dioxol-2-yl)-3-fluorobenzamide (140 mg, 0.24 mmol), Zn(CN)₂ (56 mg, 0.48 mmol), RuPhos Pd G3 (20 mg, 0.024 mmol) and X-Phos (23 mg, 0.048 mmol) in anhydrous NMP (2.0 mL) was stirred at 130° C. for 30 min under Ar. After the reaction was completed, the reaction was diluted with EA (30 mL) and washed with H₂O (15 mL×3). The organic layer was dried over sodium sulfate, filtered, and concentrated in vacuum. The residue was purified by column chromatography on silica gel (DCM/MeOH=30/1) to give 4-(4-(1-((3-cyano-7-(((S)-oxetan-2-yl)methyl)-7H-imidazo[4,5-c]pyridazin-6-yl)methyl)piperidin-4-yl)-2-methylbenzo[d][1,3]dioxol-2-yl)-3-fluorobenzamide (100 mg, yield: 70%) as a white solid. MS Calcd.: 583.2; MS Found: 584.2 [M+H]⁺.

### Step C: 3-fluoro-4-(4-(1-((3-(′-hydroxycarbamimidoyl)-7-(((S)-oxetan-2-yl)methyl)-7H-imidazo[4,5-c]pyridazin-6-yl)methyl)piperidin-4-yl)-2-methylbenzo[d][1,3]dioxol-2-yl)benzamide

To a solution of 4-(4-(1-((3-cyano-7-(((S)-oxetan-2-yl)methyl)-7H-imidazo[4,5-c]pyridazin-6-yl)methyl)piperidin-4-yl)-2-methylbenzo[d][1,3]dioxol-2-yl)-3-fluorobenzamide (100 mg, 0.17 mmol) and TEA (69 mg, 0.68 mmol) in EtOH (2 mL) was added NH₂OH HCl (24 mg, 0.34 mmol). The reaction was stirred at 80° C. for 1 hour. After the reaction was completed, the reaction was quenched with water (5 mL) and extracted with ethyl acetate (10 mL×3). The organic layer was dried over sodium sulfate, filtered and concentrated to give 3-fluoro-4-(4-(1-((3-(N′-hydroxycarbamimidoyl)-7-(((S)-oxetan-2-yl)methyl)-7H-imidazo[4,5-c]pyridazin-6-yl)methyl)piperidin-4-yl)-2-methylbenzo[d][1,3]dioxol-2-yl)benzamide (140 mg, crude) as a white solid. MS Calcd.: 616.3; MS Found: 617.1 [M+H]⁺.

### Step D: 3-fluoro-4-(2-methyl-4-(1-((7-(((S)-oxetan-2-yl)methyl)-3-(5-(trifluoromethyl)-1,2,4-oxadiazol-3-yl)-7H-imidazo[4,5-c]pyridazin-6-yl)methyl)piperidin-4-yl)benzo[d][1,3]dioxol-2-yl)benzonitrile

To a solution of 3-fluoro-4-(4-(1-((3-(N′-hydroxycarbamimidoyl)-7-(((S)-oxetan-2-yl)methyl)-7H-imidazo[4,5-c]pyridazin-6-yl)methyl)piperidin-4-yl)-2-methylbenzo[d][1,3]dioxol-2-yl)benzamide (140 mg) in THE (3.0 mL) was added TFAA (191 mg, 0.91 mmol) and the mixture stirred at rt for 2 hours. After the reaction was completed, the reaction was quenched with sodium bicarbonate aqueous solution (8.0 mL), extracted with ethyl acetate (15 mL×3). The organic layers were combined, dried over Na₂SO₄, filtered and concentrated under vacuum to dryness. The residue was purified by Prep-TLC (PE/EA=1/1) to give 3-fluoro-4-(2-methyl-4-(1-((7-(((S)-oxetan-2-yl)methyl)-3-(5-(trifluoromethyl)-1,2,4-oxadiazol-3-yl)-7H-imidazo[4,5-c]pyridazin-6-yl)methyl)piperidin-4-yl)benzo[d][1,3]dioxol-2-yl)benzonitrile (56 mg, yield: 36%) as a yellow solid. MS Calcd.: 676.2; MS Found: 677.0 [M+H]⁺.

### Step E: 3-fluoro-4-(2-methyl-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl)benzonitrile (Compound 273a)

A mixture of 3-fluoro-4-(2-methyl-4-(1-((7-(((S)-oxetan-2-yl)methyl)-3-(5-(trifluoromethyl)-1,2,4-oxadiazol-3-yl)-7H-imidazo[4,5-c]pyridazin-6-yl)methyl)piperidin-4-yl)benzo[d][1,3]dioxol-2-yl)benzonitrile (50 mg, 0.074 mmol) and N₂H₄·H₂O (11 mg, 0.22 mmol) in DMF (1.0 mL) was stirred at rt for 1 hour. After the reaction was completed, the mixture was purified directly by Prep-HPLC (0.1% FA/H₂O/CH₃CN) to give 3-fluoro-4-(2-methyl-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl)benzonitrile Compound 273a (20 mg, yield: 41%) as a white solid.

MS Calcd.: 675.2; MS Found: 676.1 [M+H]⁺.

¹H NMR (400 MHz, CD₃OD): δ 8.58 (s, 1H), 7.79 (t, J=8.0 Hz, 1H), 7.64 (d, J=11.2 Hz, 1H), 7.57 (d, J=8.0 Hz, 1H), 6.70-6.83 (m, 3H), 5.36-5.45 (m, 1H), 5.13-5.22 (m, 1H), 4.94-5.02 (m, 1H), 4.47-4.70 (m, 2H), 4.08-4.22 (m, 2H), 3.03-3.18 (m, 2H), 2.57-2.90 (m, 3H), 2.35-2.47 (m, 2H), 2.06 (s, 3H), 1.90-2.10 (m, 2H), 1.77-1.90 (m, 2H). ¹⁹F-NMR (377 MHz): −66.64, −112.09, −112.10.

### Example 226: 3-[2-({4-[2-(5-chloropyridin-2-yl)-2-methyl-2H-1,3-benzodioxol-4-yl]piperidin-1-yl}methyl)-3-[(2R)-2-methoxypropyl]-3H-imidazo[4,5-c]pyridin-6-yl]-4,5-dihydro-1,2,4-oxadiazol-5-one (Compound 427a)

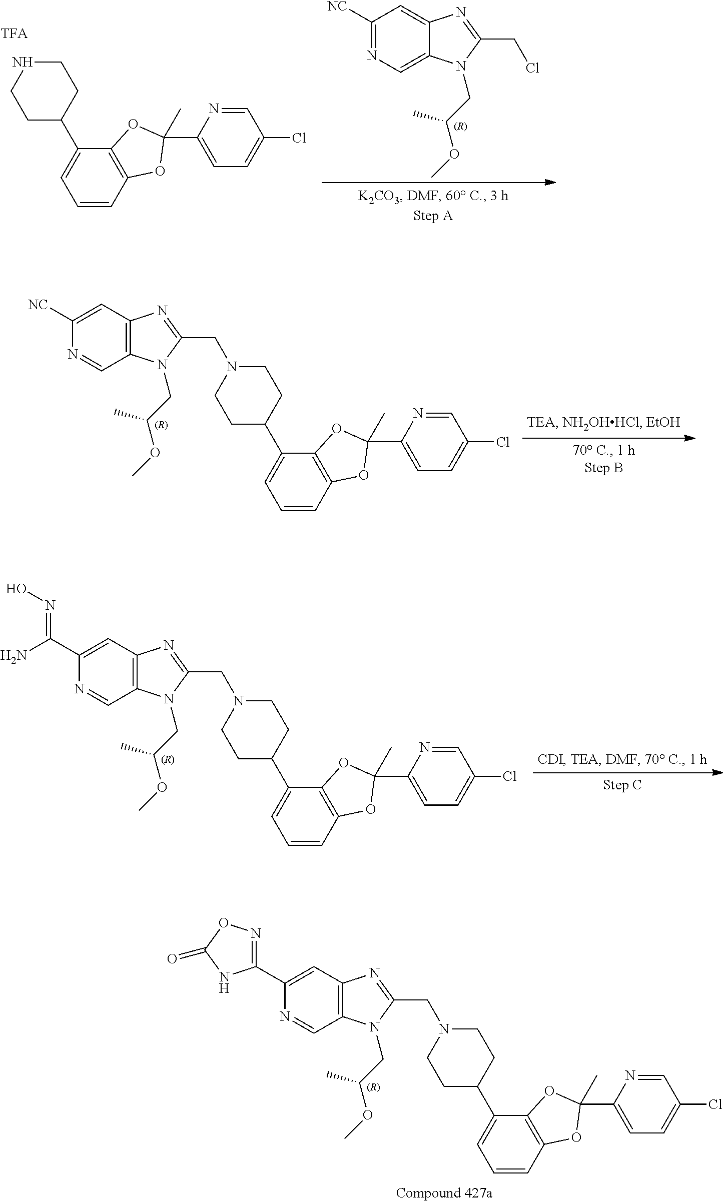

### Step A: 2-((4-(2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridine-6-carbonitrile

A mixture of 5-chloro-2-(2-methyl-4-(piperidin-4-yl)benzo[d][1,3]dioxol-2-yl)pyridine TFA salt (120 mg, 0.36 mmol) and (R)-2-(chloromethyl)-3-(2-methoxypropyl)-3H-imidazo[4,5-c]pyridine-6-carbonitrile (80 mg, 0.30 mmol), K₂CO₃ (91 mg, 0.90 mmol) in DMF (5 mL) was stirred at 60° C. for 3 hours. After the reaction was completed, the mixture was diluted with EA (60 mL), washed with H₂O (40 mL×3). The combined organic layer was dried over anhydrous sodium sulfate, filtered and evaporated to dryness. The residue was purified by column chromatography on silica gel (EA/PE=1/1) to give 2-((4-(2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridine-6-carbonitrile (85 mg, yield: 50%) as yellow oil. MS Calcd.: 558.2; MS Found: 559.2 [M+H]⁺.

### Step B: 2-((4-(2-(5-chloropyridin-2-yl)-2-methylbenzo[d][1,3]dioxol-4-yl)piperidin-1-yl)methyl)-N′-hydroxy-3-((R)-2-methoxypropyl)-3H-imidazo[4,5-c]pyridine-6-carboximidamide

A mixture of 2-((4-(2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridine-6-carbonitrile (85 mg, 0.15 mmol) and NH₂OH HCl (63 mg, 0.90 mmol), TEA (61.66 mg, 0.61 mmol) in EtOH (4 mL) was stirred at 70° C. for 1 hour. After the reaction was completed, the mixture was filtered and the solid dried to give 2-((4-(2-(5-chloropyridin-2-yl)-2-methylbenzo[d][1,3]dioxol-4-yl)piperidin-1-yl)methyl)-N′-hydroxy-3-((R)-2-methoxypropyl)-3H-imidazo[4,5-c]pyridine-6-carboximidamide (50 mg, crude) as a white solid. MS Calcd.: 591.2; MS Found: 592.2 [M+H]⁺.

### Step C: 3-[2-({4-[2-(5-chloropyridin-2-yl)-2-methyl-2H-1,3-benzodioxol-4-yl]piperidin-1-yl}methyl)-3-[(2R)-2-methoxypropyl]-3H-imidazo[4,5-c]pyridin-6-yl]-4,5-dihydro-1,2,4-oxadiazol-5-one (Compound 427a)

To a solution of 2-((4-(2-(5-chloropyridin-2-yl)-2-methylbenzo[d][1,3]dioxol-4-yl)piperidin-1-yl)methyl)-N′-hydroxy-3-((R)-2-methoxypropyl)-3H-imidazo[4,5-c]pyridine-6-carboximidamide (10 mg) in DMF (1.5 mL) was added TEA (5.15 mg, 0.051 mmol) and CDI (4.11 mg, 0.025 mmol). The resulting mixture was stirred at 70° C. for 1 hour. The solvent was removed in vacuo. The residue was purified by Prep-HPLC to give 3-[2-({4-[2-(5-chloropyridin-2-yl)-2-methyl-2H-1,3-benzodioxol-4-yl]piperidin-1-yl}methyl)-3-[(2R)-2-methoxypropyl]-3H-imidazo[4,5-c]pyridin-6-yl]-4,5-dihydro-1,2,4-oxadiazol-5-one (Compound 427a) (3.5 mg, yield: 33%) as a white solid. MS Calcd.: 617.2; MS Found: 618.2 [M+H]⁺.

¹H NMR (400 MHz, DMSO-d₆): δ 9.06 (s, 1H), 8.71 (dd, J=5.2 Hz, 2.0 Hz, 1H), 8.15 (s, 1H), 7.97-8.04 (m, 1H), 7.56-7.62 (m, 1H), 6.71-6.83 (m, 3H), 4.40-4.57 (m, 2H), 4.03-4.10 (m, 1H), 3.85-3.94 (m, 1H), 3.73-3.80 (m, 1H), 3.00-3.11 (m, 4H), 2.78-2.86 (m, 1H), 2.56-2.70 (m, 1H), 2.25-2.35 (m, 1H), 2.13-2.25 (m, 1H), 2.00 (d, J=5.2 Hz, 3H), 1.67-1.85 (m, 4H), 1.13-1.26 (m, 3H).

### Example 227: 5-chloro-2-{4-[1-({3-[(2R)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl}pyridine (Compound 428a)

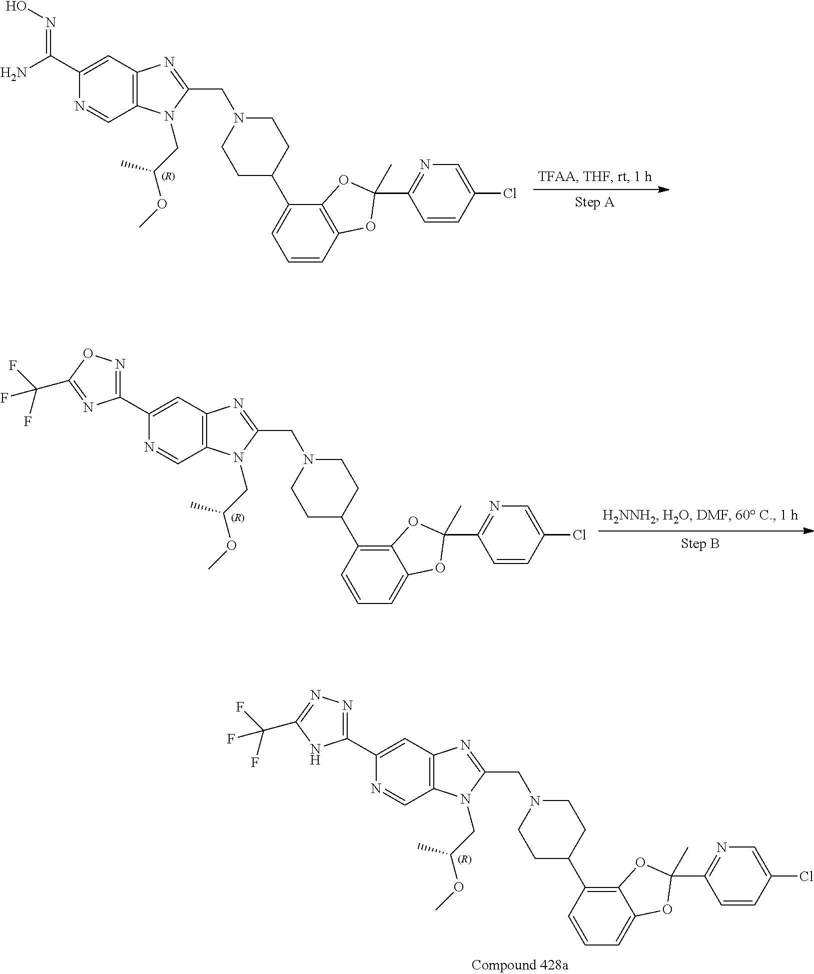

### Step A: 3-(2-((4-(2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridin-6-yl)-5-(trifluoromethyl)-1,2,4-oxadiazole

A mixture of 2-((4-(2-(5-chloropyridin-2-yl)-2-methylbenzo[d][1,3]dioxol-4-yl)piperidin-1-yl)methyl)-N′-hydroxy-3-((R)-2-methoxypropyl)-3H-imidazo[4,5-c]pyridine-6-carboximidamide (50 mg) and TFAA (36 mg, 0.17 mmol) in THF (2 mL) was stirred at rt for 1 hour. After the reaction was completed, the reaction was quenched with sat. NaHCO₃ (10 mL), extracted with ethyl acetate (15 mL×2). The organic layers were combined, dried over Na₂SO₄, filtered and concentrated under vacuum to dryness. The residue was purified by column chromatography on silica gel (DCM/MeOH=20/1) to give 3-(2-((4-(2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridin-6-yl)-5-(trifluoromethyl)-1,2,4-oxadiazole (56 mg, yield: 98%) as yellow oil. MS Calcd.: 669.2; MS Found: 670.3 [M+H]⁺.

### Step B: 5-chloro-2-{4-[1-({3-[(2R)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl}pyridine (Compound 428a)

A mixture of 3-(2-((4-(2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridin-6-yl)-5-(trifluoromethyl)-1,2,4-oxadiazole (56 mg, 0.084 mmol) and N₂H₄·H₂O (8.3 mg, 0.17 mmol) in DMF (1.5 mL) was stirred at 60° C. for 1 hour. After the reaction was completed, the mixture was purified directly by Prep-HPLC (FA) to give 5-chloro-2-{4-[1-({3-[(2R)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl}pyridine (Compound 428a) (22 mg, yield: 40%).

MS Calcd.: 668.2; MS Found: 669.2 [M+H]⁺.

¹H NMR (400 MHz, DMSO-d₆): δ 9.07 (d, J=1.2 Hz, 1H), 8.71 (dd, J=5.6 Hz, 2.4 Hz, 1H), 8.27 (s, 1H), 7.96-8.04 (m, 1H), 7.57-7.62 (m, 1H), 6.71-6.83 (m, 3H), 4.42-4.57 (m, 2H), 4.03-4.08 (m, 1H), 3.85-3.98 (m, 1H), 3.73-3.80 (m, 1H), 3.02-3.13 (m, 4H), 2.80-2.88 (m, 1H), 2.58-2.70 (m, 1H), 2.24-2.35 (m, 1H), 2.14-2.24 (m, 1H), 2.00 (d, J=5.2 Hz, 3H), 1.68-1.85 (m, 4H), 1.17-1.25 (m, 3H). ¹⁹F-NMR (377 MHz): −63.53.

### Example 228: 5-chloro-2-[(2S)-4-[1-({3-[(2R)-2-methoxypropyl]-6-(1H-1,2,3,4-tetrazol-5-yl)-3H-imidazo[4,5-c]pyridin-2-yl}methyl)piperidin-4-yl]-2-methyl-2H-1,3-benzodioxol-2-yl]pyridine (Compound 429a)

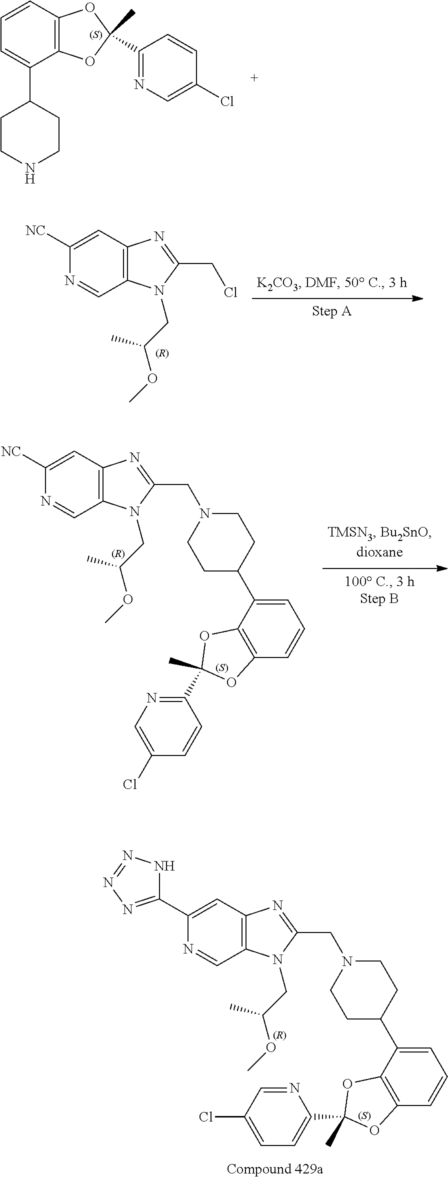

### Step A: 2-((4-((S)-2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridine-6-carbonitrile

A mixture of (S)-5-chloro-2-(2-methyl-4-(piperidin-4-yl)benzo[d][1,3]dioxol-2-yl)pyridine TFA salt (121 mg, 0.28 mmol), (R)-2-(chloromethyl)-3-(2-methoxypropyl)-3H-imidazo[4,5-c]pyridine-6-carbonitrile (50 mg, 0.18 mmol), K₂CO₃ (111 mg, 0.81 mmol) in DMF (2 mL) was stirred at 50° C. for 3 hours. After the reaction was completed, the mixture was quenched with H₂O (20 mL) and extracted with EA (10 mL*3). The organic layer was dried over anhydrous sodium sulfate, filtered and evaporated to dryness. The residue was purified by Prep-TLC to give 2-((4-((S)-2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridine-6-carbonitrile (81 mg, yield: 80%) as colorless oil. MS Calcd.: 558.2; MS Found: 558.9 [M+H]⁺.

### Step B: 5-chloro-2-[(2S)-4-[1-({3-[(2R)-2-methoxypropyl]-6-(1H-1,2,3,4-tetrazol-5-yl)-3H-imidazo[4,5-c]pyridin-2-yl}methyl)piperidin-4-yl]-2-methyl-2H-1,3-benzodioxol-2-yl]pyridine (Compound 429a)

To a mixture of 2-((4-((S)-2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridine-6-carbonitrile (81 mg, 0.14 mmol) in dioxane (2 mL) was added TMSN₃ (166 mg, 1.45 mmol) and Bu₂SnO (104 mg, 0.42 mmol). The mixture was stirred at 100° C. for 3 hours. After the reaction was completed, the mixture was purified directly by Prep-HPLC (0.03% TFA/H₂O/CH₃CN) to give 5-chloro-2-[(2S)-4-[1-({3-[(2R)-2-methoxypropyl]-6-(1H-1,2,3,4-tetrazol-5-yl)-3H-imidazo[4,5-c]pyridin-2-yl}methyl)piperidin-4-yl]-2-methyl-2H-1,3-benzodioxol-2-yl]pyridine (Compound 429a) (22 mg, yield: 26%).

MS Calcd.: 601.2; MS Found: 602.2 [M+H]⁺.

¹H NMR (400 MHz, DMSO-d₆): δ 9.11 (s, 1H), 8.71 (d, J=2.4 Hz, 1H), 8.38 (s, 1H), 7.99 (dd, J=8.4 Hz, 2.4 Hz, 1H), 7.59 (d, J=8.4 Hz, 1H), 6.71-6.83 (m, 3H), 4.42-4.58 (m, 2H), 4.08 (d, J=14.0 Hz, 1H), 3.85-3.95 (m, 1H), 3.78 (d, J=14.0 Hz, 1H), 3.02-3.11 (m, 4H), 2.80-2.88 (m, 1H), 2.60-2.70 (m, 1H), 2.15-2.35 (m, 2H), 2.01 (s, 3H), 1.70-1.88 (m, 4H), 1.21 (d, J=6.4 Hz, 3H).

### Example 229: 3-[2-({4-[(2S)-2-(5-chloropyridin-2-yl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one (Compound 430a)

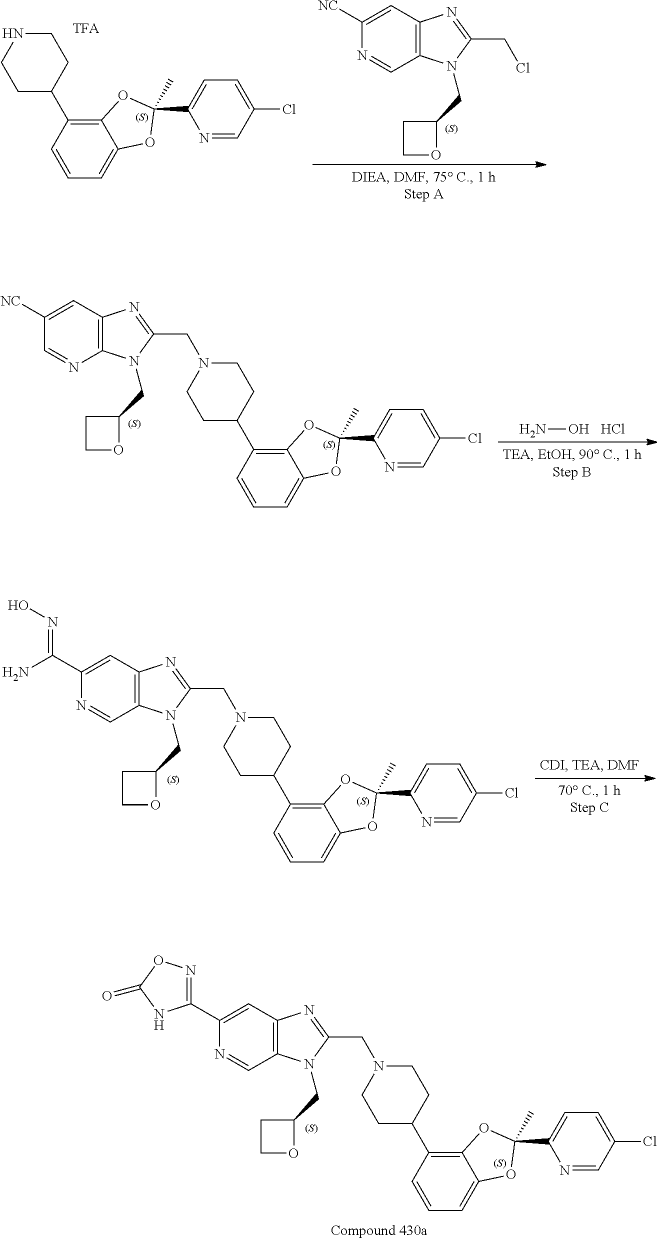

### Step A: 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile

To a solution of (S)-5-chloro-2-(2-methyl-4-(piperidin-4-yl)benzo[d][1,3]dioxol-2-yl)pyridine (67 mg, 0.151 mmol) in DMF (2 mL) was added DIEA (58 mg, 0.45 mmol). After 2 minutes, (S)-6-bromo-2-(chloromethyl)-3-(oxetan-2-ylmethyl)-3H-imidazo[4,5-c]pyridine (39 mg, 0.15 mmol) was added. The resulting mixture was heated to 75° C. for an hour. The reaction was quenched with water (10 mL), extracted with ethyl acetate (20 mL×2). The organic layers were combined, dried over Na₂SO₄, filtered and concentrated under vacuum. The residue was purified by Prep-TLC (PE:EtOAc=1:2) to give 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (60 mg, 71% yield) as a brown solid. MS Calcd.: 556.2; MS Found: 557.6 [M+H]⁺.

### Step B: 2-((4-((S)-2-(5-chloropyridin-2-yl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide

To a solution of hydroxylamine hydrochloride (15 mg, 0.216 mmol) in EtOH (2 mL) was added TEA (33 mg, 0.32 mmol). After 2 minutes, 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (60 mg, 0.11 mmol) was added. The resulting mixture was heated to 90° C. for an hour. The reaction was cooled to room temperature. The solid was collected by filtration. The solid was dried to give 2-((4-((S)-2-(5-chloropyridin-2-yl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (50 mg, 78% yield) as a white solid. MS Calcd.: 589.2; MS Found: 590.8 [M+H]⁺.

### Step C: 3-[2-({4-[(2S)-2-(5-chloropyridin-2-yl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one (Compound 430a)

To a solution of 2-((4-((S)-2-(5-chloropyridin-2-yl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (60 mg) in DMF (1.5 mL) was added CDI (33 mg, 0.203 mmol) and TEA (31 mg, 0.31 mmol). The solution was stirred at 70° C. for an hour. The solvent was removed in vacuo. The residue was purified by Prep-HPLC to afford 3-[2-({4-[(2S)-2-(5-chloropyridin-2-yl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4,5-dihydro-1,2,4-oxadiazol-5-one (Compound 430a) (27 mg, yield: 43%) as a white solid.

MS Calcd.: 615.2; MS Found: 616.2 [M+H]⁺.

¹H NMR (400 MHz, DMSO-d₆): δ 9.15 (s, 1H), 8.72 (d, J=2.4 Hz, 1H), 8.17 (s, 1H), 8.00 (dd, J=8.4 Hz, 2.4 Hz, 1H), 7.60 (d, J=8.4 Hz, 1H), 6.73-6.83 (m, 3H), 5.12-5.20 (m, 1H), 4.85-4.93 (m, 1H), 4.68-4.76 (m, 1H), 4.45-4.52 (m, 1H), 4.36-4.45 (m, 1H), 4.00 (d, J=13.6 Hz, 1H), 3.85 (d, J=14.0 Hz, 1H), 2.99-3.07 (m, 1H), 2.83-2.90 (m, 1H), 2.60-2.78 (m, 2H), 2.38-2.51 (m, 1H), 2.16-2.35 (m, 2H), 2.01 (s, 3H), 1.68-1.81 (m, 4H).

### Example 230: 6-({4-[(2S)-2-(4-chloro-2-fluorophenyl)-2-methyl-2H-1,3-benzodioxol-4-yl]piperidin-1-yl}methyl)-7-{[(2S)-oxetan-2-yl]methyl}-7H-imidazo[4,5-c]pyridazine-3-carboxylic acid (Compound 431a)

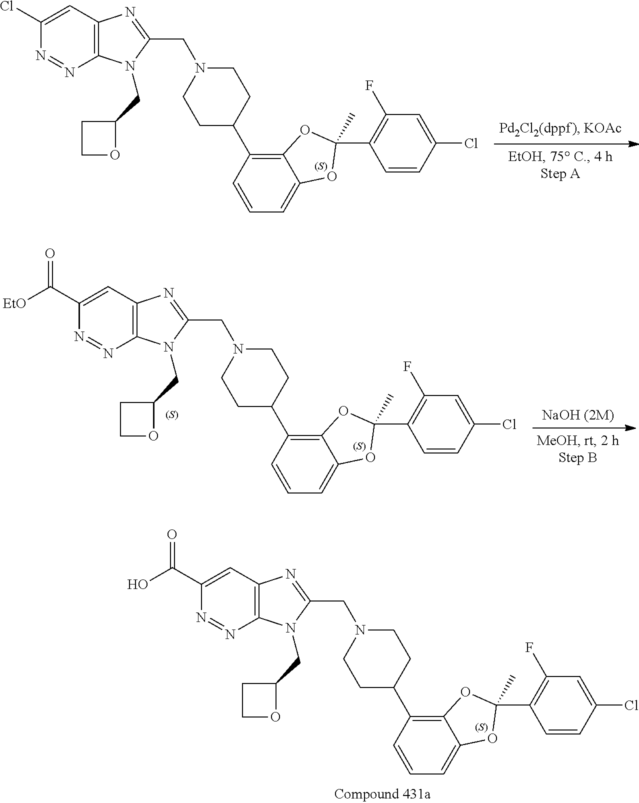

### Step A: ethyl 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate

A mixture of 3-chloro-6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine (300 mg, 0.51 mmol), KOAc (100 mg, 1.0 mmol), PdCl₂(dppf) (38 mg, 0.051 mmol) in EtOH (6.0 mL) was stirred at 75° C. for 4 hours under CO. After the reaction was completed, the mixture was purified directly by column chromatography on silica gel (DCM/MeOH=30/1) to give ethyl 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate (200 mg, yield: 62%) as a yellow solid. MS Calcd.: 621.2; MS Found: 622.1 [M+H]⁺.

### Step B: 6-({4-[(2S)-2-(4-chloro-2-fluorophenyl)-2-methyl-2H-1,3-benzodioxol-4-yl]piperidin-1-yl}methyl)-7-{[(2S)-oxetan-2-yl]methyl}-7H-imidazo[4,5-c]pyridazine-3-carboxylic acid (Compound 431a)

A mixture of ethyl 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate (80 mg, 0.13 mmol) in MeOH (2.0 mL) was added NaOH (2 M, 0.5 mL) and the mixture was stirred at rt for 2 hours. After the reaction was completed, the residue was purified directly by Prep-HPLC (0.1% FA/H₂O/CH₃CN) to give 6-({4-[(2S)-2-(4-chloro-2-fluorophenyl)-2-methyl-2H-1,3-benzodioxol-4-yl]piperidin-1-yl}methyl)-7-{[(2S)-oxetan-2-yl]methyl}-7H-imidazo[4,5-c]pyridazine-3-carboxylic acid (Compound 431a) (23 mg, yield: 30%) as a white solid.

MS Calcd.: 593.2; MS Found: 594.2 [M+H]⁺.

¹H NMR (400 MHz, DMSO-d₆): δ 8.42 (s, 1H), 7.52-7.60 (m, 2H), 7.34 (dd, J=8.4 Hz, 2.0 Hz, 1H), 6.72-6.82 (m, 3H), 5.22-5.30 (m, 1H), 4.98-5.08 (m, 1H), 4.85-4.93 (m, 1H), 4.45-4.53 (m, 1H), 4.37-4.43 (m, 1H), 4.08 (d, J=14.0 Hz, 1H), 4.02 (d, J=14.0 Hz, 1H), 2.93-3.05 (m, 2H), 2.62-2.79 (m, 2H), 2.48-2.59 (m, 1H), 2.25-2.36 (m, 2H), 2.03 (s, 3H), 1.70-1.90 (m, 4H). ¹⁹F-NMR (377 MHz): −110.78.

### Example 231: 6-({4-[(2S)-2-(4-chloro-2-fluorophenyl)-2-methyl-2H-1,3-benzodioxol-4-yl]piperidin-1-yl}methyl)-7-{[(2S)-oxetan-2-yl]methyl}-7H-imidazo[4,5-c]pyridazine-3-carboxamide (Compound 432a)

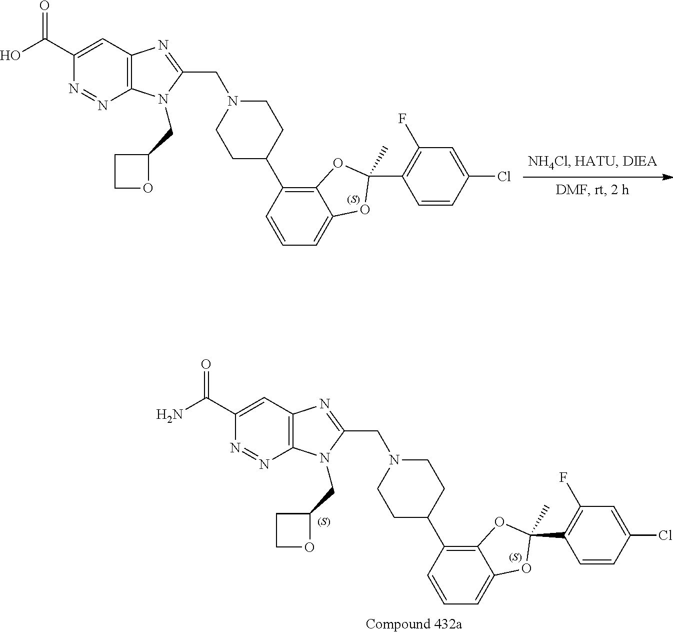

A mixture of 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylic acid (110 mg, 0.19 mmol), NH₄Cl (16 mg, 0.28 mmol), HATU (92 mg, 0.24 mmol), DIEA (72 mg, 0.56 mmol) in DMF (2.0 mL) and stirred at rt for 2 hours. After the reaction was completed, the mixture was purified directly by Prep-HPLC (0.1% NH₃·H₂O/H₂O/CH₃CN) to give 6-({4-[(2S)-2-(4-chloro-2-fluorophenyl)-2-methyl-2H-1,3-benzodioxol-4-yl]piperidin-1-yl}methyl)-7-{[(2S)-oxetan-2-yl]methyl}-7H-imidazo[4,5-c]pyridazine-3-carboxamide (Compound 432a) (35 mg, yield: 32%) as a white solid.

MS Calcd.: 592.2; MS Found: 593.2 [M+H]⁺.

¹H NMR (400 MHz, DMSO-d₆): δ 8.46 (brs, 1H), 8.39 (s, 1H), 7.85 (br s, 1H), 7.52-7.61 (m, 2H), 7.34 (dd, J=8.4 Hz, 1.6 Hz, 1H), 6.72-6.83 (m, 3H), 5.23-5.32 (m, 1H), 4.98-5.05 (m, 1H), 4.85-4.92 (m, 1H), 4.45-4.53 (m, 1H), 4.37-4.44 (m, 1H), 3.99-4.10 (m, 2H), 2.93-3.05 (m, 2H), 2.60-2.79 (m, 2H), 2.48-2.60 (m, 1H), 2.24-2.35 (m, 2H), 2.03 (s, 3H), 1.70-1.90 (m, 4H). ¹⁹F-NMR (377 MHz): −110.78.

### Example 232: 3-fluoro-4-[(2S)-2-methyl-4-{1-[(3-{[(2S)-oxetan-2-yl]methyl}-6-(5-oxo-4,5-dihydro-1,2,4-oxadiazol-3-yl)-3H-imidazo[4,5-c]pyridin-2-yl)methyl]piperidin-4-yl}-2H-1,3-benzodioxol-2-yl]benzonitrile (Compound 433a)

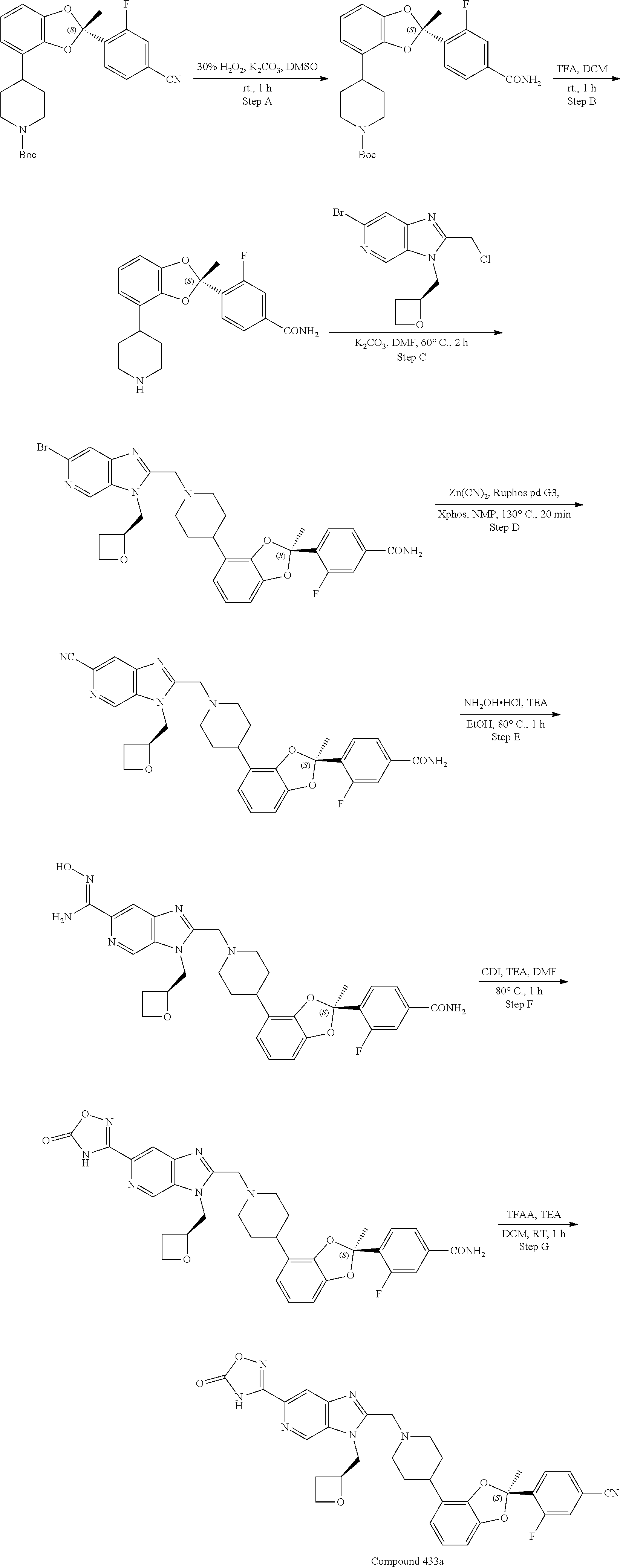

### Step A: tert-butyl (S)-4-(2-(4-carbamoyl-2-fluorophenyl)-2-methylbenzo[d][1,3]dioxol-4-yl)piperidine-1-carboxylate

To a solution of tert-butyl (S)-4-(2-(4-cyano-2-fluorophenyl)-2-methylbenzo[d][1,3]dioxol-4-yl)piperidine-1-carboxylate (2.0 g, 4.6 mmol) in DMSO (20 mL) was added 30% H₂O₂ (3 mL) and K₂CO₃ (1.9 g, 13.7 mmol). The reaction mixture was stirred at room temperature for 1 hour. After the reaction was completed, the mixture was quenched with H₂O (50 mL) and extracted with EA (30 mL*2). The combined organic layer was dried over anhydrous sodium sulfate, filtered and evaporated to dryness. The residue was purified by column chromatography on silica gel (PE/EA=1/1) to give tert-butyl (S)-4-(2-(4-carbamoyl-2-fluorophenyl)-2-methylbenzo[d][1,3]dioxol-4-yl)piperidine-1-carboxylate (2.0 g, yield: 96%) as a white solid. MS Calcd.: 456.2; MS Found: 457.3 [M+H]⁺.

### Step B: (S)-3-fluoro-4-(2-methyl-4-(piperidin-4-yl)benzo[d][1,3]dioxol-2-yl)benzamide

A mixture of tert-butyl (S)-4-(2-(4-carbamoyl-2-fluorophenyl)-2-methylbenzo[d][1,3]dioxol-4-yl)piperidine-1-carboxylate (440 mg, 0.96 mmol), TFA (1 mL) in DCM (5 mL) was stirred at room temperature for 1 hour. After the reaction was completed, the reaction mixture was filtered and dried under reduced pressure to give (S)-3-fluoro-4-(2-methyl-4-(piperidin-4-yl)benzo[d][1,3]dioxol-2-yl)benzamide (400 mg, crude) as colorless oil. MS Calcd.: 356.2; MS Found: 357.2 [M+H]⁺.

### Step C: 4-((S)-4-(1-((6-bromo-3-(((S)-oxetan-2-yl)methyl)-3H-imidazo[4,5-c]pyridin-2-yl)methyl)piperidin-4-yl)-2-methylbenzo[d][1,3]dioxol-2-yl)-3-fluorobenzamide

To a mixture of (S)-3-fluoro-4-(2-methyl-4-(piperidin-4-yl)benzo[d][1,3]dioxol-2-yl)benzamide (400 mg) in DMF (5 mL) was added K₂CO₃ (380 mg, 10.9 mmol) and (S)-6-bromo-2-(chloromethyl)-3-(oxetan-2-ylmethyl)-3H-imidazo[4,5-c]pyridine (1.5 g, 1.2 mmol). The mixture stirred at 60° C. for 2 hours. After the reaction was completed, the mixture was quenched with H₂O (100 mL) and extracted with EA (200 mL*2). The extracts were wash with brine (200 mL*3). The organic layer was dried over anhydrous sodium sulfate, filtered and evaporated to dryness. The residue was purified by column chromatography on silica gel (PE/EA=1/1) to give 4-((S)-4-(1-((6-bromo-3-(((S)-oxetan-2-yl)methyl)-3H-imidazo[4,5-c]pyridin-2-yl)methyl)piperidin-4-yl)-2-methylbenzo[d][1,3]dioxol-2-yl)-3-fluorobenzamide (400 mg, yield: 56.25%) as brown oil. MS Calcd.: 635.2; MS Found: 636.2 [M+H]⁺.

### Step D: 4-((S)-4-(1-((6-cyano-3-(((S)-oxetan-2-yl)methyl)-3H-imidazo[4,5-c]pyridin-2-yl)methyl)piperidin-4-yl)-2-methylbenzo[d][1,3]dioxol-2-yl)-3-fluorobenzamide

To a solution of 4-((S)-4-(1-((6-bromo-3-(((S)-oxetan-2-yl)methyl)-3H-imidazo[4,5-c]pyridin-2-yl)methyl)piperidin-4-yl)-2-methylbenzo[d][1,3]dioxol-2-yl)-3-fluorobenzamide (350 mg, 0.551 mmol) in NMP (3 mL) was added Zn(CN)₂ (194 mg, 1.65 mmol), Ruphos Pd G3 (46 mg, 0.055 mmol), Xphos (26 mg, 0.055 mmol) under N₂. The mixture was stirred at 130° C. for 20 min under N₂ (1 atm). After the reaction was completed, the mixture was quenched with H₂O (50 mL) and extracted with EA (30 mL*2). The combined organic layer was dried over anhydrous sodium sulfate, filtered and evaporated to dryness. The residue was purified by column chromatography on silica gel (DCM/MeOH=20/1) to give 4-((S)-4-(1-((6-cyano-3-(((S)-oxetan-2-yl)methyl)-3H-imidazo[4,5-c]pyridin-2-yl)methyl)piperidin-4-yl)-2-methylbenzo[d][1,3]dioxol-2-yl)-3-fluorobenzamide (100 mg, yield: 31%) as a brown solid. MS Calcd.: 582.2; MS Found: 583.3 [M+H]⁺.

### Step E: 3-fluoro-4-((S)-4-(1-((6-(N′-hydroxycarbamimidoyl)-3-(((S)-oxetan-2-yl)methyl)-3H-imidazo[4,5-c]pyridin-2-yl)methyl)piperidin-4-yl)-2-methylbenzo[d][1,3]dioxol-2-yl)benzamide

To a mixture of 4-((S)-4-(1-((6-cyano-3-(((S)-oxetan-2-yl)methyl)-3H-imidazo[4,5-c]pyridin-2-yl)methyl)piperidin-4-yl)-2-methylbenzo[d][1,3]dioxol-2-yl)-3-fluorobenzamide (100 mg, 0.17 mmol) in EtOH (5 mL) was added hydroxylamine hydrochloride (29 mg, 0.43 mmol) and TEA (104 mg, 1.0 mmol). The mixture was stirred at rt for 1 h and then heated to 80° C. for 1 hours. After the reaction was completed, the reaction mixture was filtered. The solid obtained was dried to give 3-fluoro-4-((S)-4-(1-((6-(N′-hydroxycarbamimidoyl)-3-(((S)-oxetan-2-yl)methyl)-3H-imidazo[4,5-c]pyridin-2-yl)methyl)piperidin-4-yl)-2-methylbenzo[d][1,3]dioxol-2-yl)benzamide (140 mg, crude) as a white solid. MS Calcd.: 615.3; MS Found: 616.4 [M+H]⁺.

### Step F: 3-fluoro-4-((S)-2-methyl-4-(1-((3-(((S)-oxetan-2-yl)methyl)-6-(5-oxo-4,5-dihydro-1,2,4-oxadiazol-3-yl)-3H-imidazo[4,5-c]pyridin-2-yl)methyl)piperidin-4-yl)benzo[d][1,3]dioxol-2-yl)benzamide

A mixture of 3-fluoro-4-((S)-4-(1-((6-(N′-hydroxycarbamimidoyl)-3-(((S)-oxetan-2-yl)methyl)-3H-imidazo[4,5-c]pyridin-2-yl)methyl)piperidin-4-yl)-2-methylbenzo[d][1,3]dioxol-2-yl)benzamide (140 mg), CDI (73 mg, 0.45 mmol), TEA (91 mg, 0.91 mmol) in DMF (2 mL) was stirred at 80° C. for 1 hour. After the reaction was completed, the mixture was diluted with H₂O (50 mL), extracted with EA (50 mL*2). The combined organic layer was dried over anhydrous sodium sulfate, filtered and evaporated to dryness. The residue was purified by column chromatography on silica gel (PE/EA=1/9) to give 3-fluoro-4-((S)-2-methyl-4-(1-((3-(((S)-oxetan-2-yl)methyl)-6-(5-oxo-4,5-dihydro-1,2,4-oxadiazol-3-yl)-3H-imidazo[4,5-c]pyridin-2-yl)methyl)piperidin-4-yl)benzo[d][1,3]dioxol-2-yl)benzamide (47 mg, yield: 33%) as white oil. MS Calcd.: 641.2; MS Found: 642.3 [M+H]⁺.

### Step G: 3-fluoro-4-[(2S)-2-methyl-4-{1-[(3-{[(2S)-oxetan-2-yl]methyl}-6-(5-oxo-4,5-dihydro-1,2,4-oxadiazol-3-yl)-3H-imidazo[4,5-c]pyridin-2-yl)methyl]piperidin-4-yl}-2H-1,3-benzodioxol-2-yl]benzonitrile (Compound 433a)

A mixture of 3-fluoro-4-((S)-2-methyl-4-(1-((3-(((S)-oxetan-2-yl)methyl)-6-(5-oxo-4,5-dihydro-1,2,4-oxadiazol-3-yl)-3H-imidazo[4,5-c]pyridin-2-yl)methyl)piperidin-4-yl)benzo[d][1,3]dioxol-2-yl)benzamide (47 mg, 0.073 mmol), TFAA (153 mg, 0.733 mmol), TEA (14 mg, 0.146 mmol) in DCM (1 mL) was stirred at RT for 1 hour. After the reaction was completed, the mixture was purified directly by Prep-HPLC (0.03% TFA/H₂O/CH₃CN) to give 3-fluoro-4-[(2S)-2-methyl-4-{1-[(3-{[(2S)-oxetan-2-yl]methyl}-6-(5-oxo-4,5-dihydro-1,2,4-oxadiazol-3-yl)-3H-imidazo[4,5-c]pyridin-2-yl)methyl]piperidin-4-yl}-2H-1,3-benzodioxol-2-yl]benzonitrile (Compound 433a) (3.3 mg, yield: 7.4%). MS Calcd.: 623.2; MS Found: 624.2 [M+H]⁺.

¹H NMR (400 MHz, DMSO-d₆): δ 9.13 (s, 1H), 8.11-8.19 (m, 2H), 7.98 (d, J=11.2 Hz, 1H), 7.70-7.78 (m, 2H), 6.71-6.85 (m, 3H), 5.11-5.20 (m, 1H), 4.85-4.95 (m, 1H), 4.70-4.78 (m, 1H), 4.36-4.54 (m, 2H), 4.01 (d, J=14.0 Hz, 1H), 3.85 (d, J=13.6 Hz, 1H), 3.00-3.07 (m, 1H), 2.84-2.93 (m, 1H), 2.60-2.80 (m, 2H), 2.15-2.35 (m, 3H), 2.05 (s, 3H), 1.67-1.82 (m, 4H). ¹⁹F-NMR (377 MHz): −111.09.

### Example 233: 4-[(2S)-2-(4-chloro-2-fluorophenyl)-2-methyl-2H-1,3-benzodioxol-4-yl]-1-[(4-methyl-3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 302a)

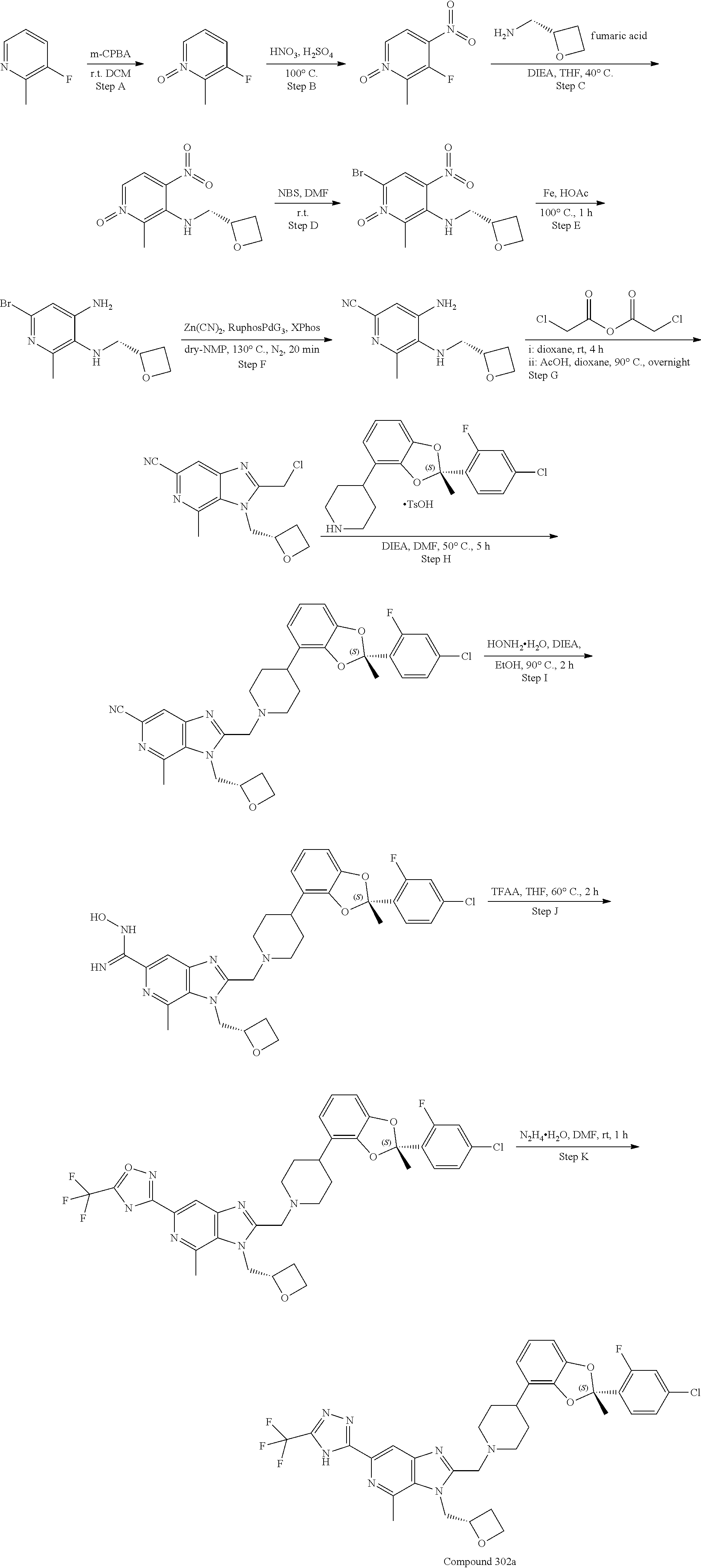

### Step A: 3-fluoro-2-methylpyridine 1-oxide

To a solution of 3-fluoro-2-methylpyridine (2.5 g, 22.5 mmol) in DCM (25 mL) was added m-CPBA (5.49 g, 27.02 mmol) at 0° C. The reaction was stirred at room temperature for 4 h. The resulting mixture was concentrated, and the residue purified by column chromatography on silica gel (DCM:MeOH=10:1) to give 3-fluoro-2-methylpyridine 1-oxide (1.3 g, 46% yield) as a white solid. MS Calcd.: 127.0; MS Found: 128.1 [M+H]⁺.

### Step B: 3-fluoro-2-methyl-4-nitropyridine 1-oxide

To a solution of 3-fluoro-2-methylpyridine 1-oxide (1.33 g, 10.5 mmol) in con·H₂SO₄ (5 mL) was added HNO₃ (9.2 mL) at room temperature. The mixture was heated to 100° C. and stirred for 2 h. The reaction mixture was cooled down to room temperature, and then poured into ice. The pH value of the aqueous layer was adjusted to 5˜6 using Sat.NaHCO₃. The aqueous phase was extracted with DCM (50 mL×3). The combined organic layer was concentrated, and the residue purified by column chromatography on silica gel (DCM=100%) to give 3-fluoro-2-methyl-4-nitropyridine 1-oxide (727 mg, 40% yield) as a yellow solid. MS Calcd.: 172.0; MS Found: 173.1 [M+H]⁺.

### Step C: (S)-2-methyl-4-nitro-3-((oxetan-2-ylmethyl)amino)pyridine 1-oxide

A mixture of 3-fluoro-2-methyl-4-nitropyridine 1-oxide (624 mg, 3.63 mmol), (S)-oxetan-2-ylmethanamine fumaric acid salt (685 mg, 4.72 mmol), DIEA (1.4 g, 10.9 mmol) in THE (13 mL) was stirred at 40° C. overnight. The resulting mixture was diluted with EA (50 mL) and washed with water (50 mL*2). The organic layer was dried over Na₂SO₄, filtered and concentrated. The residue was purified by column chromatography on silica gel (DCM/MeOH=50/1) to give (S)-2-methyl-4-nitro-3-((oxetan-2-ylmethyl)amino)pyridine 1-oxide (676 mg, 78% yield) as a white solid. MS Calcd.: 239.1; MS Found: 240.1 [M+H]⁺.

### Step D: (S)-6-bromo-2-methyl-4-nitro-3-((oxetan-2-ylmethyl)amino)pyridine 1-oxide

To a solution of (S)-2-methyl-4-nitro-3-((oxetan-2-ylmethyl)amino)pyridine 1-oxide (200 mg, 0.836 mmol) in DMF (2 mL) was added NBS (164 mg, 0.92 mmol) at room temperature. The mixture was stirred for 3 h, diluted with EA (50 mL) and wished with water (50 mL×3). The organic layer was dried over Na₂SO₄, filtered and evaporated to dryness. The residue was purified by column chromatography on silica gel (DCM:MeOH=20:1) to give (S)-6-bromo-2-methyl-4-nitro-3-((oxetan-2-ylmethyl)amino)pyridine 1-oxide (238 mg, 89% yield) as an orange solid. MS Calcd.: 317.0; MS Found: 318.0 [M+H]⁺.

### Step E: (S)-6-bromo-2-methyl-N3-(oxetan-2-ylmethyl)pyridine-3,4-diamine

A mixture of (S)-6-bromo-2-methyl-4-nitro-3-((oxetan-2-ylmethyl)amino)pyridine 1-oxide (320 mg, 1.01 mmol), iron powder (377 mg, 6.74 mmol) in AcOH (12 mL) was stirred at 100° C. for 1 h. The resulting mixture was filtered and pH value was adjusted to 7-8 with Sat.NaHCO₃. The mixture was extracted with EA (20 mL×3). The combined organic layer was concentrated, and the residue purified by column chromatography on silica gel (DCM:MeOH=20:1) to give (S)-6-bromo-2-methyl-N3-(oxetan-2-ylmethyl)pyridine-3,4-diamine (190 mg, 70.1% yield) as an orange solid. MS Calcd.: 271.0; MS Found: 272.0 [M+H]⁺.

### Step F: (S)-4-amino-6-methyl-5-((oxetan-2-ylmethyl)amino)picolinonitrile

A mixture of (S)-6-bromo-2-methyl-N3-(oxetan-2-ylmethyl)pyridine-3,4-diamine (308 mg, 1.13 mmol), Zn(CN)₂ (198 mg, 1.69 mmol), Ruphos Pd G3 (95 mg, 0.113 mmol) and Xphos (54 mg, 0.113 mmol) in NMP (3 mL) was stirred at 130° C. for 20 min under N₂. After the reaction was completed, the mixture was diluted with EA (70 mL), washed with H₂O (40 mL). The organic layer was dried over anhydrous sodium sulfate, filtered and evaporated to dryness. The residue was purified by column chromatography on silica gel (DCM/MeOH=30/1) to give (S)-4-amino-6-methyl-5-((oxetan-2-ylmethyl)amino)picolinonitrile (148 mg, 60% yield) as an orange solid. MS Calcd.: 218.1; MS Found: 219.3 [M+H]⁺.

### Step G: (S)-2-(chloromethyl)-4-methyl-3-(oxetan-2-ylmethyl)-3H-imidazo[4,5-c]pyridine-6-carbonitrile

A mixture of (S)-4-amino-6-methyl-5-((oxetan-2-ylmethyl)amino)picolinonitrile (650 mg, 3.0 mmol) and 2-chloroacetic anhydride (765 mg, 4.5 mmol) in dioxane (20 mL) was stirred at rt for 4 hrs. After the reaction was completed, the mixture was diluted with EA (100 mL), washed with aqueous NaHCO₃ (50 mL*2). The organic layer was dried over anhydrous sodium sulfate, filtered and evaporated in vacuo to dryness. The residue was dissolved in dioxane (20 mL) and AcOH (0.8 mL) was added. The mixture was stirred at 90° C. overnight. The mixture was diluted with EA (100 mL), washed with aqueous NaHCO₃ (50 mL*2). The organic layer was dried over anhydrous sodium sulfate, filtered and concentrated. The residue was purified by column chromatography on silica gel (EA/PE=1/1) to give (S)-2-(chloromethyl)-4-methyl-3-(oxetan-2-ylmethyl)-3H-imidazo[4,5-c]pyridine-6-carbonitrile (265 mg, yield: 32%) as yellow oil. MS Calcd.: 276.1; MS Found: 277.0 [M+H]⁺.

### Step H: 2-((4-((S)-2-(4-chloro-2-fluorophenyl)-2-methylbenzo[d][1,3]dioxol-4-yl)piperidin-1-yl)methyl)-4-methyl-3-(((S)-oxetan-2-yl)methyl)-3H-imidazo[4,5-c]pyridine-6-carbonitrile

A solution of (S)-4-(2-(4-chloro-2-fluorophenyl)-2-methylbenzo[d][1,3]dioxol-4-yl)piperidine TsOH salt (225 mg, 0.43 mmol), (S)-2-(chloromethyl)-4-methyl-3-(oxetan-2-ylmethyl)-3H-imidazo[4,5-c]pyridine-6-carbonitrile (120 mg, 0.43 mmol), DIEA (166 mg, 1.29 mmol) in DMF (3 mL) was stirred at 50° C. for 5 hours. After the reaction was completed, the mixture was diluted with EA (50 mL), washed with H₂O (40 mL*3). The combined organic layer was dried over anhydrous sodium sulfate, filtered and concentrated. The residue was purified by column chromatography on silica gel (DCM/MeOH=50/1) to give 2-((4-((S)-2-(4-chloro-2-fluorophenyl)-2-methylbenzo[d][1,3]dioxol-4-yl)piperidin-1-yl)methyl)-4-methyl-3-(((S)-oxetan-2-yl)methyl)-3H-imidazo[4,5-c]pyridine-6-carbonitrile (150 mg, yield: 59%) as yellow oil. MS Calcd.: 587.2; MS Found: 588.1 [M+H]⁺.

### Step I: 2-((4-((S)-2-(4-chloro-2-fluorophenyl)-2-methylbenzo[d][1,3]dioxol-4-yl)piperidin-1-yl)methyl)-N-hydroxy-4-methyl-3-(((S)-oxetan-2-yl)methyl)-3H-imidazo[4,5-c]pyridine-6-carboximidamide

A solution of 2-((4-((S)-2-(4-chloro-2-fluorophenyl)-2-methylbenzo[d][1,3]dioxol-4-yl)piperidin-1-yl)methyl)-4-methyl-3-(((S)-oxetan-2-yl)methyl)-3H-imidazo[4,5-c]pyridine-6-carbonitrile (150 mg, 0.26 mmol), HONH₂·HCl (36 mg, 0.51 mmol), DIEA (168 mg, 1.30 mmol) in EtOH (4 mL) was stirred at 90° C. for 2 h. The precipitate was collected by filtration and washed with EtOH (1 mL). The solid was dried under vacuum to give 2-((4-((S)-2-(4-chloro-2-fluorophenyl)-2-methylbenzo[d][1,3]dioxol-4-yl)piperidin-1-yl)methyl)-N-hydroxy-4-methyl-3-(((S)-oxetan-2-yl)methyl)-3H-imidazo[4,5-c]pyridine-6-carboximidamide (60 mg, 39% yield) as a white solid. MS Calcd.: 620.2; MS Found: 621.1 [M+H]⁺.

### Step J: 3-(2-((4-((S)-2-(4-chloro-2-fluorophenyl)-2-methylbenzo[d][1,3]dioxol-4-yl)piperidin-1-yl)methyl)-4-methyl-3-(((S)-oxetan-2-yl)methyl)-3H-imidazo[4,5-c]pyridin-6-yl)-5-(trifluoromethyl)-1,2,4-oxadiazole

A solution of 2-((4-((S)-2-(4-chloro-2-fluorophenyl)-2-methylbenzo[d][1,3]dioxol-4-yl)piperidin-1-yl)methyl)-N-hydroxy-4-methyl-3-(((S)-oxetan-2-yl)methyl)-3H-imidazo[4,5-c]pyridine-6-carboximidamide (60 mg, 0.1 mmol), TFAA (3 drops) in THE (3 mL) was stirred at 60° C. for 2 h. After the reaction was completed, the mixture was diluted with EA (20 mL), washed with Sat. NaHCO₃ (20 mL). The organic layer was dried over anhydrous sodium sulfate, filtered and concentrated. The residue was purified by Prep-TLC (EA=100%) to give 3-(2-((4-((S)-2-(4-chloro-2-fluorophenyl)-2-methylbenzo[d][1,3]dioxol-4-yl)piperidin-1-yl)methyl)-4-methyl-3-(((S)-oxetan-2-yl)methyl)-3H-imidazo[4,5-c]pyridin-6-yl)-5-(trifluoromethyl)-1,2,4-oxadiazole (58 mg, yield: 83%) as colorless oil. MS Calcd.: 698.2; MS Found: 699.3 [M+H]⁺.

### Step K: 4-[(2S)-2-(4-chloro-2-fluorophenyl)-2-methyl-2H-1,3-benzodioxol-4-yl]-1-[(4-methyl-3-1{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 302a)

A mixture of 3-(2-((4-((S)-2-(4-chloro-2-fluorophenyl)-2-methylbenzo[d][1,3]dioxol-4-yl)piperidin-1-yl)methyl)-4-methyl-3-(((S)-oxetan-2-yl)methyl)-3H-imidazo[4,5-c]pyridin-6-yl)-5-(trifluoromethyl)-1,2,4-oxadiazole (58 mg, 0.08 mmol), hydrazine hydrate (1 drop) in DMF (1 mL) was stirred at rt for 1 hour. After the reaction was completed, the mixture was filtered and the filtrate was purified by Prep-HPLC to afford 4-[(2S)-2-(4-chloro-2-fluorophenyl)-2-methyl-2H-1,3-benzodioxol-4-yl]-1-[(4-methyl-3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 302a) (27 mg, yield: 46%) as a white solid.

MS Calcd.: 697.2; MS Found: 698.3 [M+H]⁺.

¹H NMR (400 MHz, DMSO-d₆): δ 8.14 (s, 1H), 7.52-7.60 (m, 2H), 7.34 (dd, J=8.4 Hz, 1.6 Hz, 1H), 6.72-6.81 (m, 3H), 5.15-5.23 (m, 1H), 5.02-5.11 (m, 1H), 4.80-4.87 (m, 1H), 4.48-4.56 (m, 1H), 4.37-4.44 (m, 1H), 4.05 (d, J=14.0 Hz, 1H), 3.83 (d, J=13.2 Hz, 1H), 3.00-3.08 (m, 1H), 2.99 (s, 3H), 2.86-2.93 (m, 1H), 2.72-2.83 (m, 1H), 2.60-2.72 (m, 1H), 2.42-2.51 (m, 1H), 2.18-2.35 (m, 2H), 2.03 (s, 3H), 1.63-1.88 (m, 4H). ¹⁹F-NMR (377 MHz): −63.71, −110.77.

### Example 234: 3-[2-({4-[(2S)-2-(4-chloro-2-fluorophenyl)-2-methyl-2H-1,3-benzodioxol-4-yl]piperidin-1-yl}methyl)-4-methyl-3-{[(2S)-oxetan-2-yl]methyl}-3H-imidazo[4,5-c]pyridin-6-yl]-4,5-dihydro-1,2,4-oxadiazol-5-one (Compound 434a)

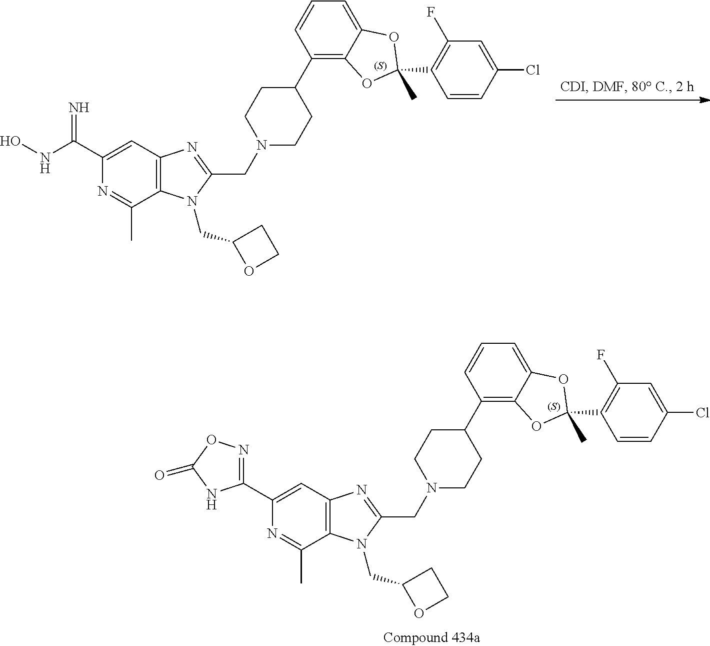

A mixture of 2-((4-((S)-2-(4-chloro-2-fluorophenyl)-2-methylbenzo[d][1,3]dioxol-4-yl)piperidin-1-yl)methyl)-N-hydroxy-4-methyl-3-(((S)-oxetan-2-yl)methyl)-3H-imidazo[4,5-c]pyridine-6-carboximidamide (50 mg, 0.08 mmol), CDI (20 mg, 0.12 mmol), TEA (16 mg, 0.16 mmol) in DMF (1 mL) was stirred at 80° C. for 2 hours. After the reaction was completed, the mixture was diluted with EA (30 mL), washed with H₂O (20 mL*3). The combined organic layer was dried over anhydrous sodium sulfate, filtered and evaporated to dryness. The residue was purified by Prep-HPLC to afford 3-[2-({4-[(2S)-2-(4-chloro-2-fluorophenyl)-2-methyl-2H-1,3-benzodioxol-4-yl]piperidin-1-yl}methyl)-4-methyl-3-{[(2S)-oxetan-2-yl]methyl}-3H-imidazo[4,5-c]pyridin-6-yl]-4,5-dihydro-1,2,4-oxadiazol-5-one (Compound 434a) (27 mg, yield: 52%) as a white solid.

MS Calcd.: 646.2; MS Found: 647.1 [M+H]⁺.

¹H NMR (400 MHz, DMSO-d₆): δ 8.02 (s, 1H), 7.53-7.60 (m, 2H), 7.34 (dd, J=8.4 Hz, 2.0 Hz, 1H), 6.71-6.83 (m, 3H), 5.14-5.22 (m, 1H), 5.00-5.09 (m, 1H), 4.79-4.86 (m, 1H), 4.47-4.55 (m, 1H), 4.34-4.42 (m, 1H), 4.04 (d, J=14.0 Hz, 1H), 3.83 (d, J=13.6 Hz, 1H), 3.00-3.13 (m, 1H), 2.94 (s, 3H), 2.86-2.93 (m, 1H), 2.60-2.82 (m, 2H), 2.40-2.54 (m, 1H), 2.17-2.35 (m, 2H), 2.03 (s, 3H), 1.62-1.88 (m, 4H). ¹⁹F-NMR (377 MHz): −110.76.

### Example 235: 2-[(4-chloro-2-fluorophenyl)methoxy]-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 435a)

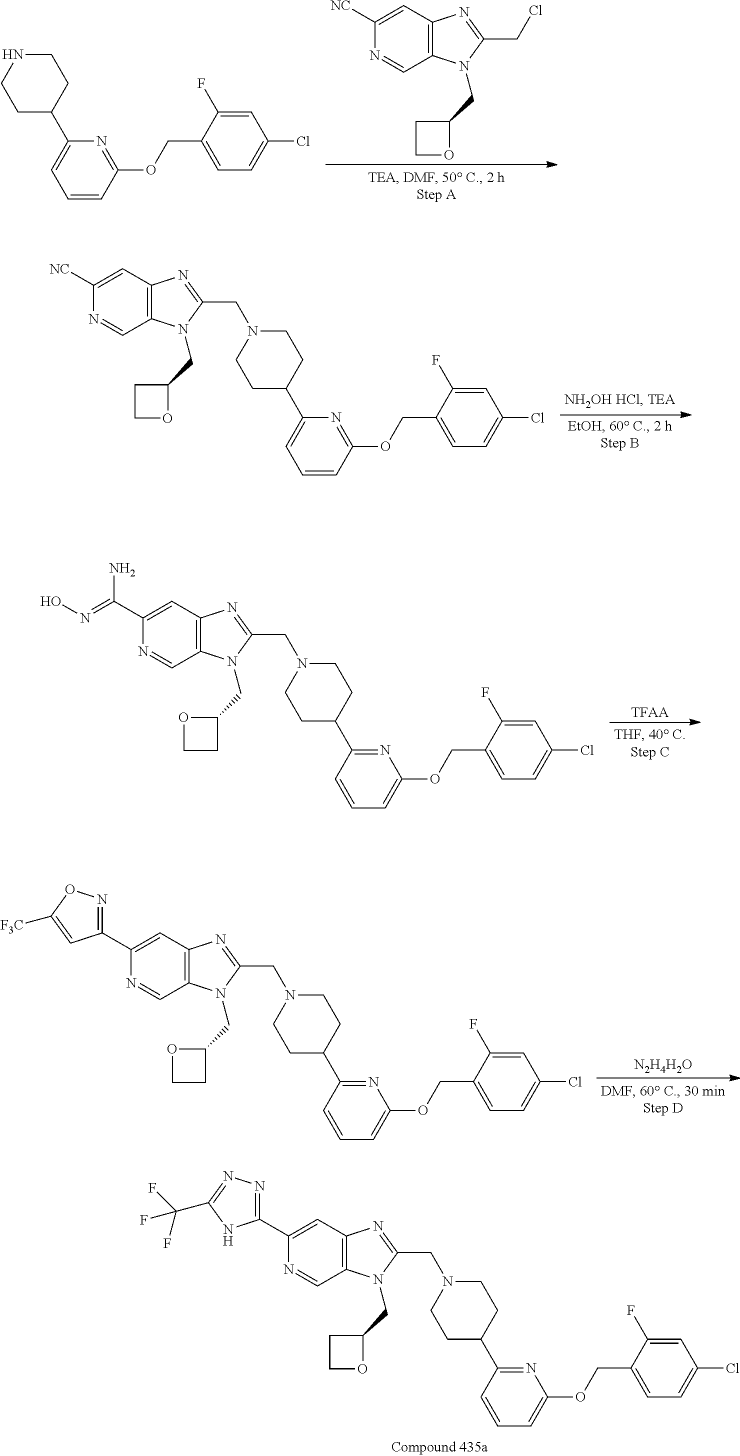

### Step A: (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carbonitrile

A mixture of 2-((4-chloro-2-fluorobenzyl)oxy)-6-(piperidin-4-yl)pyridine (60 mg, 0.167 mmol), (S)-2-(chloromethyl)-3-(oxetan-2-ylmethyl)-3H-imidazo[4,5-c]pyridine-6-carbonitrile (40 mg, 0.15 mmol) and TEA (92 mg, 0.91 mmol) in DMF (1 mL) was stirred at 50° C. for 2 h. The resulting mixture was diluted with H₂O (25 mL), extracted with EA (10 mL×3). The combined organic layer was dried over anhydrous Na₂SO4, filtered and concentrated. The residue was purified by column chromatography on silica gel (DCM:MeOH=20:1) to give (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carbonitrile (90 mg, crude, ca. 100%) as a white solid. MS Calcd.: 546.2; MS Found: 547.4 [M+H]⁺.

### Step B: (S)-2-((4-(6-((4-chloro-2-fluorobenzyl)oxy)pyridin-2-yl)piperidin-1-yl)methyl)-N′-hydroxy-3-(oxetan-2-ylmethyl)-3H-imidazo[4,5-c]pyridine-6-carboximidamide

A mixture of (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carbonitrile (90 mg, 0.16 mmol), NH₂OH·HCl (46 mL, 0.66 mmol), TEA (100 mg, 0.98 mmol) in EtOH (1 mL) was stirred at 60° C. for 2 h. The resulting mixture was diluted with H₂O (20 mL) and extracted with EA (10 mL×3). The combined organic layer was dried over anhydrous Na₂SO₄, filtered and concentrated to give (S)-2-((4-(6-((4-chloro-2-fluorobenzyl)oxy)pyridin-2-yl)piperidin-1-yl)methyl)-N′-hydroxy-3-(oxetan-2-ylmethyl)-3H-imidazo[4,5-c]pyridine-6-carboximidamide (60 mg, crude, 63% yield) as a white solid. MS Calcd.: 579.2; MS Found: 580.2 [M+H]⁺.

### Step C: (S)-3-(2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridin-6-yl)-5-(trifluoromethyl)-1,2,4-oxadiazole

To a mixture of (S)-2-((4-(6-((4-chloro-2-fluorobenzyl)oxy)pyridin-2-yl)piperidin-1-yl)methyl)-N′-hydroxy-3-(oxetan-2-ylmethyl)-3H-imidazo[4,5-c]pyridine-6-carboximidamide (60 mg) in THE (1 mL) was added TFAA (87 mg, 0.41 mmol) at rt. The resulting mixture was stirred at 40° C. for 2 h, then diluted with EA (5 mL). Sat.NaHCO₃ (about 10 mL) was added, and the organic layer was dried over anhydrous Na₂SO₄, filtered and concentrated. The residue was purified by Prep-TLC (DCM:MeOH=20:1) to give (S)-3-(2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridin-6-yl)-5-(trifluoromethyl)-1,2,4-oxadiazole (32 mg, 47% yield) as a white solid. MS Calcd.: 657.2; MS Found: 658.1 [M+H]⁺.

### Step D: 2-[(4-chloro-2-fluorophenyl)methoxy]-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 435a)

To a mixture of (S)-3-(2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridin-6-yl)-5-(trifluoromethyl)-1,2,4-oxadiazole (32 mg, 0.049 mmol) in DMF (0.5 mL) was added N₂H₄·H₂O (10 mg, 0.196 mmol). The resulting mixture was stirred at 60° C. for 30 min, purified directly by Prep-HPLC (0.1% FA) to give 2-[(4-chloro-2-fluorophenyl)methoxy]-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 435a) (6.6 mg, 20% yield) as a white solid.

MS Calcd.: 656.2; MS Found: 657.3 [M+H]⁺.

¹H NMR (400 MHz, DMSO-d₆): δ 9.31 (s, 1H), 8.40 (s, 1H), 7.70 (t, J=8.0 Hz, 1H), 7.60 (t, J=8.0 Hz, 1H), 7.47 (d, J=10.0 Hz, 1H), 7.32 (d, J=7.6 Hz, 1H), 6.95 (d, J=7.2 Hz, 1H), 6.75 (d, J=8.0 Hz, 1H), 5.41 (s, 2H), 5.05-5.15 (m, 1H), 4.73-5.00 (m, 4H), 4.47-4.56 (m, 1H), 4.38-4.45 (m, 1H), 3.67-3.83 (m, 2H), 3.25-3.40 (m, 2H), 2.89-3.03 (m, 1H), 2.70-2.83 (m, 1H), 2.32-2.43 (m, 1H), 2.05-2.20 (m, 4H). ¹⁹F-NMR (377 MHz): −63.73, −115.18.

### Example 236: 2-[(4-chloro-2-fluorophenyl)methoxy]-6-{1-[(3-{[(2S)-oxetan-2-yl]methyl}-6-(4H-1,2,4-triazol-3-yl)-3H-imidazo[4,5-c]pyridin-2-yl)methyl]piperidin-4-yl}pyridine (Compound 436a)

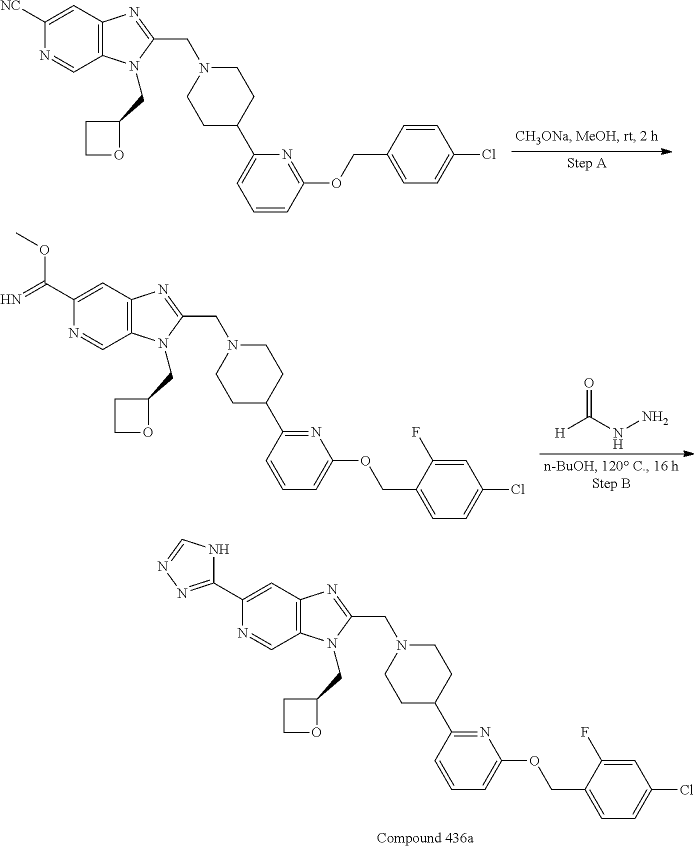

### Step A: methyl (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carbimidate

To a solution of (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carbonitrile (200 mg, 0.37 mmol) in MeOH (5 mL) was added CH₃ONa (198 mg, 3.7 mmol). The reaction was stirred at room temperature for 2 hours. After the reaction was completed, the mixture was concentrated and purified by column chromatography on silica gel (PE:EA=1:1) to give methyl (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carbimidate (200 mg, 95% yield) as a white solid. MS Calcd.: 578.2; MS Found: 579.8 [M+H]⁺.

### Step B: 2-[(4-chloro-2-fluorophenyl)methoxy]-6-{1-[(3-{[(2S)-oxetan-2-yl]methyl}-6-(4H-1,2,4-triazol-3-yl)-3H-imidazo[4,5-c]pyridin-2-yl)methyl]piperidin-4-yl}pyridine (Compound 436a)

To a solution of methyl (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carbimidate (150 mg, 0.26 mmol) in n-BuOH (2 mL) was added formohydrazide (62 mg, 1.0 mmol) and DIEA (134 mg, 1.0 mmol). The reaction was stirred at 120° C. for 16 hours. After the reaction was completed, the reaction was filtered and purified by Prep-HPLC to give 2-[(4-chloro-2-fluorophenyl)methoxy]-6-{1-[(3-{[(2S)-oxetan-2-yl]methyl}-6-(4H-1,2,4-triazol-3-yl)-3H-imidazo[4,5-c]pyridin-2-yl)methyl]piperidin-4-yl}pyridine (Compound 436a) (65 mg, 43% yield) as a white solid.

MS Calcd.: 588.2; MS Found: 589.3 [M+H]⁺.

¹H NMR (400 MHz, DMSO-d₆): δ 9.28 (s, 1H), 8.40 (s, 1H), 8.28 (s, 1H), 7.71 (t, J=8.0 Hz, 1H), 7.61 (t, J=8.0 Hz, 1H), 7.49 (dd, J=10.0 Hz, J=1.6 Hz, 1H), 7.33 (dd, J=8.0 Hz, J=2.0 Hz, 1H), 6.96 (d, J=7.2 Hz, 1H), 6.76 (d, J=8.0 Hz, 1H), 5.42 (s, 2H), 5.07-5.15 (m, 1H), 4.86-5.00 (m, 3H), 4.73-4.82 (m, 1H), 4.47-4.56 (m, 1H), 4.38-4.45 (m, 1H), 3.70-3.90 (m, 2H), 3.28-3.45 (m, 2H), 2.90-3.05 (m, 1H), 2.70-2.82 (m, 1H), 2.30-2.43 (m, 1H), 2.03-2.22 (m, 4H). ¹⁹F-NMR (377 MHz): −115.19.

### Example 237: 3-[2-({4-[2-(5-chloropyridin-2-yl)-2-methyl-2H-1,3-benzodioxol-4-yl]piperidin-1-yl}methyl)-3-[(2S)-2-methoxypropyl]-3H-imidazo[4,5-c]pyridin-6-yl]-4,5-dihydro-1,2,4-oxadiazol-5-one (Compound 437a)

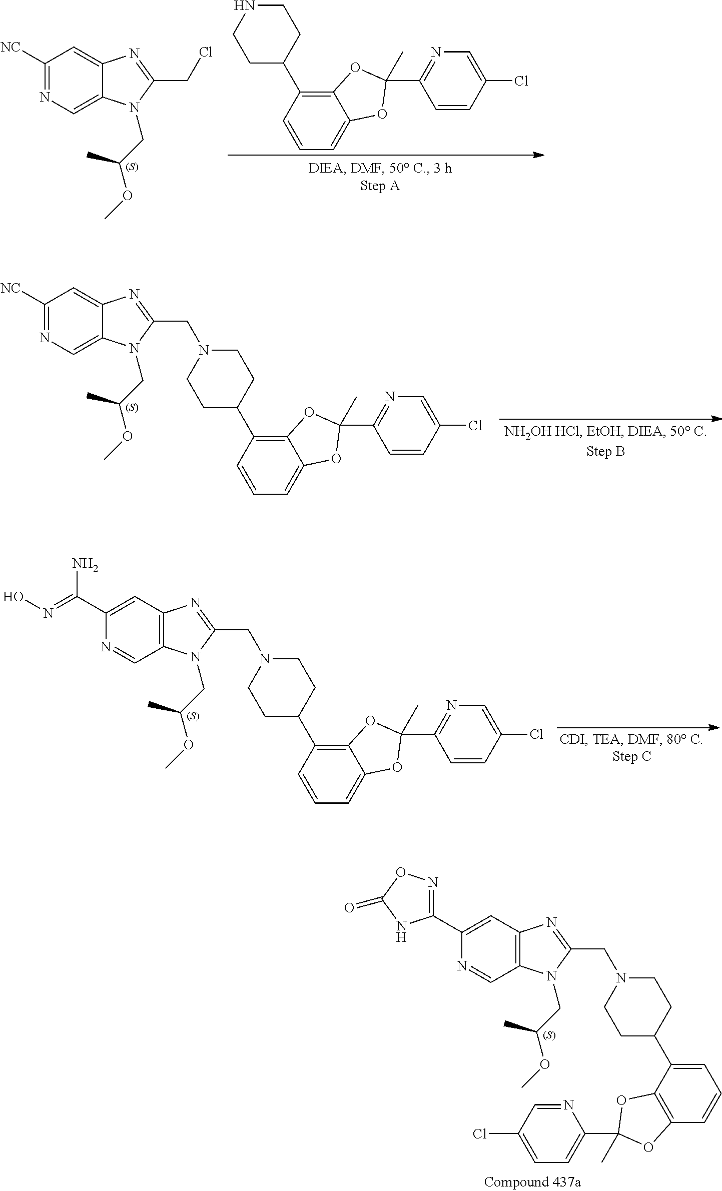

### Step A: 2-((4-(2-(5-chloropyridin-2-yl)-2-methylbenzo[d][1,3]dioxol-4-yl)piperidin-1-yl)methyl)-3-((S)-2-methoxypropyl)-3H-imidazo[4,5-c]pyridine-6-carbonitrile

A mixture of (S)-2-(chloromethyl)-3-(2-methoxypropyl)-3H-imidazo[4,5-c]pyridine-6-carbonitrile (100 mg, 0.38 mmol), 5-chloro-2-(2-methyl-4-(piperidin-4-yl)benzo[d][1,3]dioxol-2-yl)pyridine (124 mg, 0.38 mmol) and DIEA (147 mg, 1.14 mmol) in DMF (4 mL) was stirred at 50° C. for 3 hours. After the reaction was completed, the mixture was diluted with ethyl acetate (20 mL), washed with brine (15 mL×2). The organic layer was dried over sodium sulfate, filtered and concentrated in vacuum. The residue was purified by column chromatography on silica gel (DCM/MeOH=30/1) to give 2-((4-(2-(5-chloropyridin-2-yl)-2-methylbenzo[d][1,3]dioxol-4-yl)piperidin-1-yl)methyl)-3-((S)-2-methoxypropyl)-3H-imidazo[4,5-c]pyridine-6-carbonitrile (202 mg, yield: 95%) as yellow oil. MS Calcd.: 558.2; MS Found: 559.3 [M+H]⁺.

### Step B: 2-((4-(2-(5-chloropyridin-2-yl)-2-methylbenzo[d][1,3]dioxol-4-yl)piperidin-1-yl)methyl)-N′-hydroxy-3-((S)-2-methoxypropyl)-3H-imidazo[4,5-c]pyridine-6-carboximidamide

A mixture of 2-((4-(2-(5-chloropyridin-2-yl)-2-methylbenzo[d][1,3]dioxol-4-yl)piperidin-1-yl)methyl)-3-((S)-2-methoxypropyl)-3H-imidazo[4,5-c]pyridine-6-carbonitrile (202 mg, 0.36 mmol), hydroxylamine hydrochloride (50 mg, 0.72 mmol) and DIEA (232 mg, 1.8 mmol) in EtOH (5 mL) was stirred at 50° C. for 6 hours. The mixture was diluted with EA (15 mL), washed with brine (15 mL xl). The organic layer was dried over sodium sulfate, filtered and concentrated under reduced pressure. The residue was purified by Prep-TLC (DCM/MeOH=10/1) to give 2-((4-(2-(5-chloropyridin-2-yl)-2-methylbenzo[d][1,3]dioxol-4-yl)piperidin-1-yl)methyl)-N′-hydroxy-3-((S)-2-methoxypropyl)-3H-imidazo[4,5-c]pyridine-6-carboximidamide (180 mg, yield: 84%) as white solid. MS Calcd.: 591.2; MS Found: 592.2 [M+H]⁺.

### Step C: 3-[2-({4-[2-(5-chloropyridin-2-yl)-2-methyl-2H-1,3-benzodioxol-4-yl]piperidin-1-yl}methyl)-3-[(2S)-2-methoxypropyl]-3H-imidazo[4,5-c]pyridin-6-yl]-4,5-dihydro-1,2,4-oxadiazol-5-one (Compound 437a)

A mixture of 2-((4-(2-(5-chloropyridin-2-yl)-2-methylbenzo[d][1,3]dioxol-4-yl)piperidin-1-yl)methyl)-N′-hydroxy-3-((S)-2-methoxypropyl)-3H-imidazo[4,5-c]pyridine-6-carboximidamide (62 mg, 0.15 mmol), CDI (102 mg, 0.63 mmol) and TEA (46 mg, 0.45 mmol) in DMF (2 mL) was stirred at 80° C. for 3 hours. The reaction mixture was purified by prep-HPLC(FA) directly to give 3-[2-({4-[2-(5-chloropyridin-2-yl)-2-methyl-2H-1,3-benzodioxol-4-yl]piperidin-1-yl}methyl)-3-[(2S)-2-methoxypropyl]-3H-imidazo[4,5-c]pyridin-6-yl]-4,5-dihydro-1,2,4-oxadiazol-5-one (Compound 437a) (23 mg, yield: 14%) as white solid.

MS Calcd.: 617.2; MS Found: 618.4 [M+H]⁺.

¹H NMR (400 MHz, DMSO-d₆): δ 9.05 (d, J=1.2 Hz, 1H), 8.72 (dd, J=5.6 Hz, 2.4 Hz, 1H), 8.14 (s, 1H), 7.98-8.04 (m, 1H), 7.59 (dd, J=8.0 Hz, 6.0 Hz, 1H), 6.75-6.83 (m, 2H), 6.70-6.75 (m, 1H), 4.40-4.55 (m, 2H), 4.02-4.17 (m, 1H), 3.83-3.95 (m, 1H), 3.73-3.80 (m, 1H), 3.00-3.10 (m, 4H), 2.79-2.86 (m, 1H), 2.58-2.70 (m, 1H), 2.10-2.35 (m, 2H), 2.00 (d, J=4.8 Hz, 3H), 1.67-1.85 (m, 4H), 1.19 (t, J=6.0 Hz, 3H).

### Example 238: 3-{2-[(4-{6-[(4-chloro-2-fluorophenyl)methoxy]-5-fluoropyridin-2-yl}piperidin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 438a)

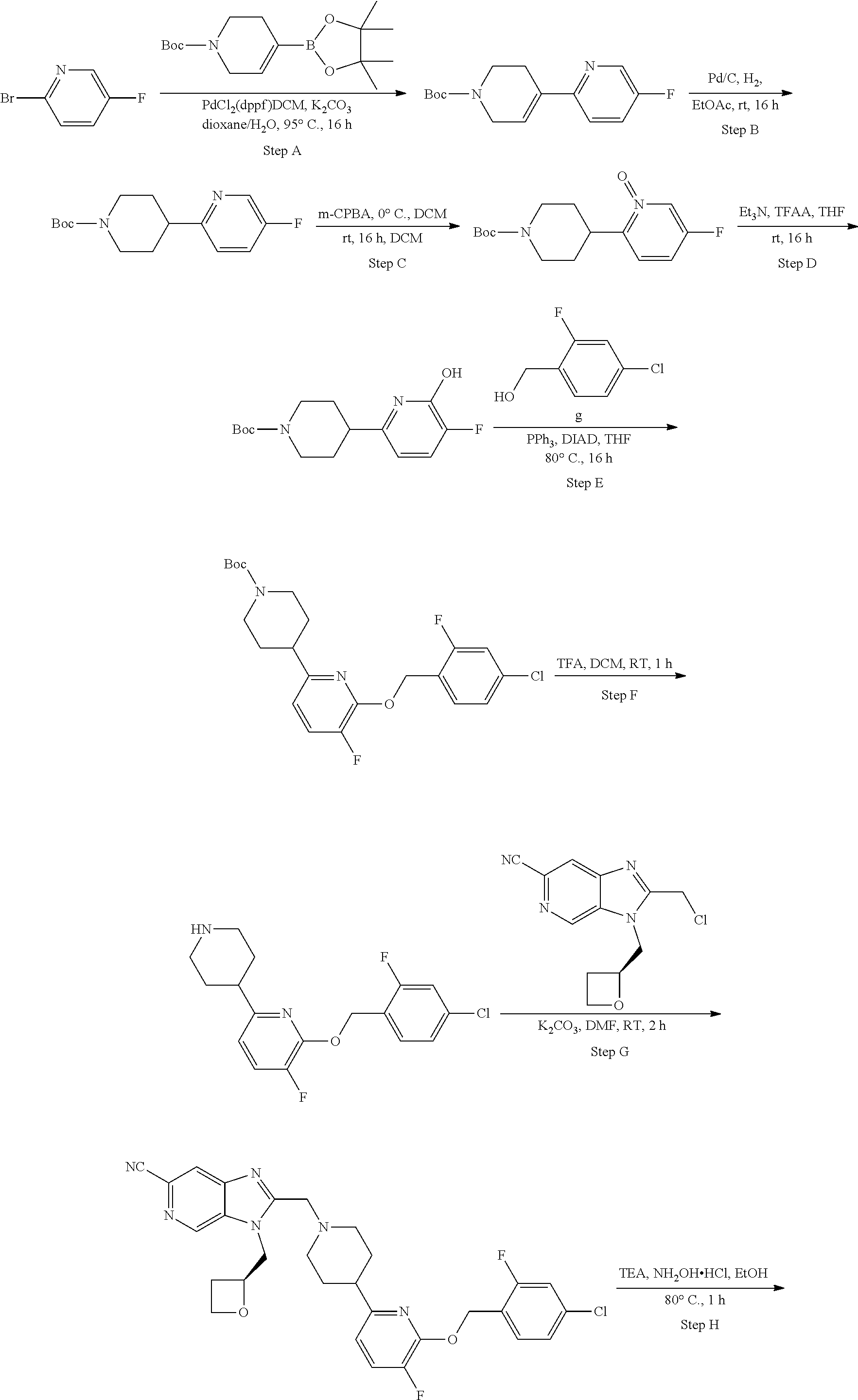

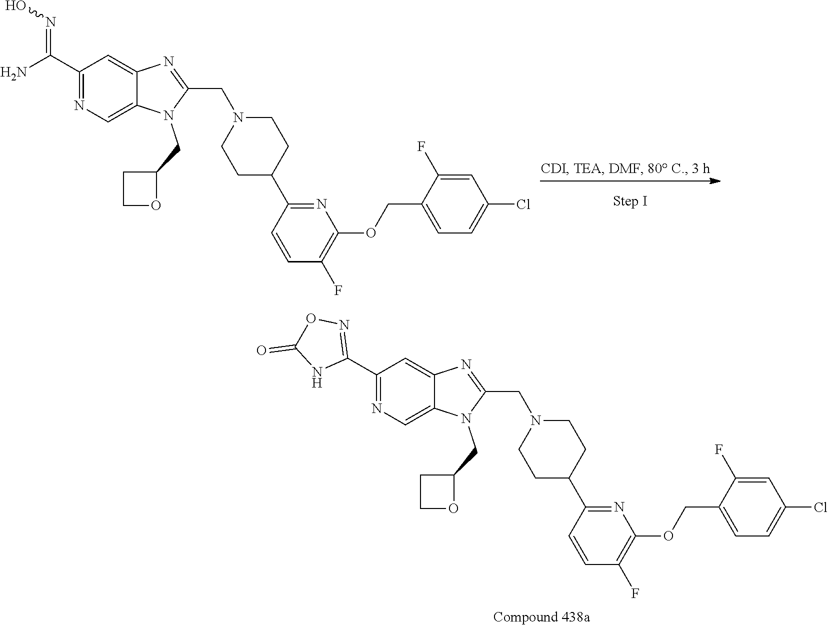

### Step A: tert-butyl 5-fluoro-3′,6′-dihydro-[2,4′-bipyridine]-1′(2′H)-carboxylate

To a solution of 2-bromo-5-fluoropyridine (10 g, 56.82 mmol) in dioxane (500 mL) and H₂O (50 mL) was added tert-butyl 4-(4,4,5,5-tetramethyl-1,3,2-dioxaborolan-2-yl)-3,6-dihydropyridine-1(2H)-carboxylate (19.3 g, 62.4 mmol), Pd(dppf)Cl₂·DCM (4.8 g, 5.7 mmol) and K₂CO₃ (23.5 g, 170.3 mmol). The reaction mixture was stirred at 95° C. for 16 hours under N₂. After the reaction was completed, the mixture was quenched with H₂O (1 L) and extracted with EA (500 mL*3). The combined organic layer was dried over anhydrous sodium sulfate, filtered and evaporated to dryness. The residue was purified by column chromatography on silica gel (PE/EA=10/1) to give tert-butyl 5-fluoro-3′,6′-dihydro-[2,4′-bipyridine]-1′(2′H)-carboxylate (15.0 g, yield: 96%) as yellow oil. MS Calcd.: 278.1; MS Found: 279.3 [M+H]⁺.

### Step B: tert-butyl 4-(5-fluoropyridin-2-yl)piperidine-1-carboxylate

A mixture of tert-butyl 5-fluoro-3′,6′-dihydro-[2,4′-bipyridine]-1′(2′H)-carboxylate (3 g, 10.71 mmol), Pd/C (300 mg, 10% on Carbon, wetted with ca. 55% water) in EtOAc (5 mL) was stirred at room temperature for 16 hours under H₂ (1 atm). After the reaction was completed, the reaction mixture was filtered and the filtrate evaporated under reduced pressure to give tert-butyl 4-(5-fluoropyridin-2-yl)piperidine-1-carboxylate (2.8 g, yield: 94%) as colorless oil. MS Calcd.: 280.2; MS Found: 281.3 [M+H]⁺.

### Step C: 2-(1-(tert-butoxycarbonyl)piperidin-4-yl)-5-fluoropyridine 1-oxide

To a mixture of tert-butyl 4-(5-fluoropyridin-2-yl)piperidine-1-carboxylate (2.8 g) in DCM (30 mL) was added m-CPBA (4.3 g, 25.1 mmol) at 0° C. The mixture stirred at room temperature for 16 hours. After the reaction was completed, the mixture was quenched with H₂O (200 mL) and extracted with EA (100 mL*3). The extracts were wash with brine (200 mL*3). The organic layer was dried over anhydrous sodium sulfate, filtered and evaporated to dryness. The residue was purified by column chromatography on silica gel (PE/EA=1/1) to give 2-(1-(tert-butoxycarbonyl)piperidin-4-yl)-5-fluoropyridine 1-oxide (1.2 g, yield: 40%) as yellow oil. MS Calcd.: 296.2; MS Found: 241.2 [M-56+H]⁺.

### Step D: tert-butyl 4-(5-fluoro-6-hydroxypyridin-2-yl)piperidine-1-carboxylate

To a solution of 2-(1-(tert-butoxycarbonyl)piperidin-4-yl)-5-fluoropyridine 1-oxide (1.2 g, 4.05 mmol) in THE (30 mL) was added TFAA (8.5 g, 40.5 mmol), Et₃N (820 mg, 8.11 mmol). The mixture was stirred at room temperature for 16 h. After the reaction was completed, the mixture was quenched with H₂O (100 mL) and extracted with EA (100 mL*2). The combined organic layer was dried over anhydrous sodium sulfate, filtered and evaporated to dryness. The residue was purified by column chromatography on silica gel (DCM/MeOH=20/1) to give tert-butyl 4-(5-fluoro-6-hydroxypyridin-2-yl)piperidine-1-carboxylate (644 mg, yield: 54%) as a yellow solid. MS Calcd.: 296.2; MS Found: 241.2 [M-56+H]⁺.

### Step E: tert-butyl 4-(6-((4-chloro-2-fluorobenzyl)oxy)-5-fluoropyridin-2-yl)piperidine-1-carboxylate

To a mixture of tert-butyl 4-(5-fluoro-6-hydroxypyridin-2-yl)piperidine-1-carboxylate (644 mg, 2.2 mmol) in THE (10 mL) was added (4-chloro-2-fluorophenyl)methanol (523 mg, 3.3 mmol), PPh₃ (854 mg, 3.3 mmol) and DIAD (658 mg, 3.3 mmol). The mixture was stirred at 80° C. for 16 h. After the reaction was completed, the reaction mixture was evaporated and the residue was purified by prep-TLC (PE/EA=2/1) to give tert-butyl 4-(6-((4-chloro-2-fluorobenzyl)oxy)-5-fluoropyridin-2-yl)piperidine-1-carboxylate (700 mg, yield: 74%) as yellow oil. MS Calcd.: 438.2; MS Found: 383.0 [M-56+H]⁺.

### Step F: 2-((4-chloro-2-fluorobenzyl)oxy)-3-fluoro-6-(piperidin-4-yl)pyridine

A mixture of tert-butyl 4-(6-((4-chloro-2-fluorobenzyl)oxy)-5-fluoropyridin-2-yl)piperidine-1-carboxylate (200 mg, 0.52 mmol) and TFA (0.5 mL) in DCM (1.5 mL) was stirred at room temperature for 1 h. After the reaction was completed, the mixture was evaporated to give 2-((4-chloro-2-fluorobenzyl)oxy)-3-fluoro-6-(piperidin-4-yl)pyridine TFA salt (150 mg, crude) as yellow oil. MS Calcd.: 338.1; MS Found: 339.2 [M+H]⁺.

### Step G: (S)-2-((4-(6-((4-chloro-2-fluorobenzyl)oxy)-5-fluoropyridin-2-yl)piperidin-1-yl)methyl)-3-(oxetan-2-ylmethyl)-3H-imidazo[4,5-c]pyridine-6-carbonitrile

A mixture of 2-((4-chloro-2-fluorobenzyl)oxy)-3-fluoro-6-(piperidin-4-yl)pyridine TFA salt (150 mg), (S)-2-(chloromethyl)-3-(oxetan-2-ylmethyl)-3H-imidazo[4,5-c]pyridine-6-carbonitrile (40 mg, 0.15 mmol), K₂CO₃ (500 mg, 3.62 mmol) in DMF (5 mL) was stirred at R^{T} for 2 hours. After the reaction was completed, the mixture was quenched with H₂O (100 mL) and extracted with EA (100 mL*2). The combined organic layer was dried over anhydrous sodium sulfate, filtered and evaporated to dryness. The residue was purified by prep-TLC to give (S)-2-((4-(6-((4-chloro-2-fluorobenzyl)oxy)-5-fluoropyridin-2-yl)piperidin-1-yl)methyl)-3-(oxetan-2-ylmethyl)-3H-imidazo[4,5-c]pyridine-6-carbonitrile (50 mg, yield: 57%). MS Calcd.: 564.2; MS Found: 565.3 [M+H]⁺.

### Step H: (S)-2-((4-(6-((4-chloro-2-fluorobenzyl)oxy)-5-fluoropyridin-2-yl)piperidin-1-yl)methyl)-N′-hydroxy-3-(oxetan-2-ylmethyl)-3H-imidazo[4,5-c]pyridine-6-carboximidamide

A mixture of (S)-2-((4-(6-((4-chloro-2-fluorobenzyl)oxy)-5-fluoropyridin-2-yl)piperidin-1-yl)methyl)-3-(oxetan-2-ylmethyl)-3H-imidazo[4,5-c]pyridine-6-carbonitrile (50 mg, 0.085 mmol), hydroxylamine hydrochloride (14.5 mg, 0.21 mmol), TEA (52 mg, 0.51 mmol) in EtOH (5 mL) was stirred at 80° C. for 1 h. After the reaction was completed, the mixture was diluted with H₂O (50 mL), extracted with EA (50 mL*2). The combined organic layer was dried over anhydrous sodium sulfate, filtered and evaporated to dryness. The residue was purified by prep-TLC to give (S)-2-((4-(6-((4-chloro-2-fluorobenzyl)oxy)-5-fluoropyridin-2-yl)piperidin-1-yl)methyl)-N′-hydroxy-3-(oxetan-2-ylmethyl)-3H-imidazo[4,5-c]pyridine-6-carboximidamide (40 mg, yield: 80%) as white oil. MS Calcd.: 597.2; MS Found: 598.3 [M+H]⁺.

### Step I: 3-{2-[(4-{6-[(4-chloro-2-fluorophenyl)methoxy]-5-fluoropyridin-2-yl}piperidin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 438a)

A mixture of (S)-2-((4-(6-((4-chloro-2-fluorobenzyl)oxy)-5-fluoropyridin-2-yl)piperidin-1-yl)methyl)-N′-hydroxy-3-(oxetan-2-ylmethyl)-3H-imidazo[4,5-c]pyridine-6-carboximidamide (40 mg, 0.067 mmol), CDI (21 mg, 0.134 mmol), TEA (27 mg, 0.268 mmol) in DMF (1 mL) was stirred at 80° C. for 3 hours. After the reaction was completed, the mixture was purified directly by Prep-HPLC (0.03% TFA/H₂O/CH₃CN) to give 3-{2-[(4-{6-[(4-chloro-2-fluorophenyl)methoxy]-5-fluoropyridin-2-yl}piperidin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 438a) (7.4 mg, yield: 18%).

MS Calcd.: 623.2; MS Found: 624.1 [M+H]⁺.

¹H NMR (400 MHz, DMSO-d₆): δ 9.16 (s, 1H), 8.17 (s, 1H), 7.54-7.62 (m, 2H), 7.48 (dd, J=10.0 Hz, 2.0 Hz, 1H), 7.32 (dd, J=8.4 Hz, 2.0 Hz, 1H), 6.91 (dd, J=8.0 Hz, 2.8 Hz, 1H), 5.47 (s, 2H), 5.12-5.21 (m, 1H), 4.88-4.97 (m, 1H), 4.72-4.80 (m, 1H), 4.38-4.54 (m, 2H), 4.01 (d, J=13.6 Hz, 1H), 3.86 (d, J=13.6 Hz, 1H), 2.98-3.05 (m, 1H), 2.82-2.90 (m, 1H), 2.57-2.79 (m, 2H), 2.40-2.55 (m, 1H), 2.16-2.35 (m, 2H), 1.60-1.85 (m, 4H). ¹⁹F-NMR (377 MHz): −115.07, −143.99.

### Example 239: 3-{2-[(4-{3-[(4-chloro-2-fluorophenyl)methoxy]-4-fluorophenyl}piperazin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 439a)

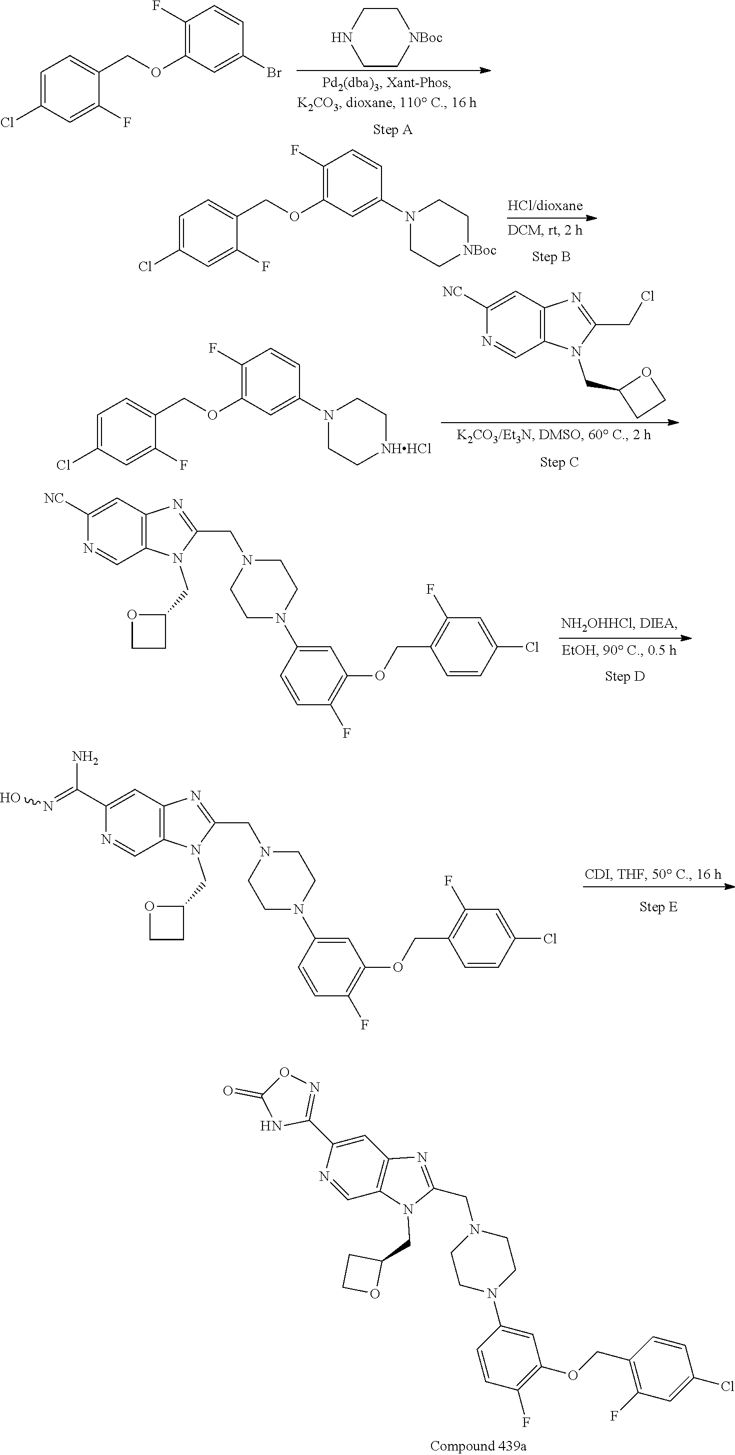

### Step A: tert-butyl 4-(3-((4-chloro-2-fluorobenzyl)oxy)-4-fluorophenyl)piperazine-1-carboxylate

A mixture of 4-bromo-2-((4-chloro-2-fluorobenzyl)oxy)-1-fluorobenzene (500 mg, 1.51 mmol), tert-butyl piperazine-1-carboxylate (281 mg, 1.51 mmol), Pd₂dba₃ (137 mg, 0.15 mmol), XantPhos (173 mg, 0.30 mmol) and K₂CO₃ (623 mg, 4.52 mmol) in 1,4-dioxane (10 mL) was stirred at 110° C. under Ar for 16 h. The mixture was poured into water (200 mL) and extracted with EtOAc (2×200 mL), the combined organic layer was washed with brine, dried over sodium sulfate, filtered and the residue was concentrated. The residue was purified by column chromatography on silica gel to obtain tert-butyl 4-(3-((4-chloro-2-fluorobenzyl)oxy)-4-fluorophenyl)piperazine-1-carboxylate (230 mg, yield: 34.9%) as a yellow oil. MS Calcd: 438.2; MS Found: 439.1 [M+H]⁺.

### Step B: 1-(3-((4-chloro-2-fluorobenzyl)oxy)-4-fluorophenyl)piperazine hydrochloride

A mixture of tert-butyl 4-(3-((4-chloro-2-fluorobenzyl)oxy)-4-fluorophenyl)piperazine-1-carboxylate (230 mg, 0.525 mmol) and hydrochloric acid 1,4-dioxane solution (5 mL, 4M) in DCM (5 mL) was stirred at rt for 2 h. The mixture was concentrated to obtain 1-(3-((4-chloro-2-fluorobenzyl)oxy)-4-fluorophenyl)piperazine hydrochloride (197 mg, crude) as a yellow solid. MS Calcd: 338.1; MS Found: 339.1 [M+H]⁺.

### Step C: (S)-2-((4-(3-((4-chloro-2-fluorobenzyl)oxy)-4-fluorophenyl)piperazin-1-yl)methyl)-3-(oxetan-2-ylmethyl)-3H-imidazo[4,5-c]pyridine-6-carbonitrile

A mixture of 1-(3-((4-chloro-2-fluorobenzyl)oxy)-4-fluorophenyl)piperazine hydrochloride (170 mg), K₂CO₃ (188 mg, 1.36 mmol) and TEA (137 mg, 1.36 mmol) in DMSO (2 mL) was stirred at rt for 30 min. (S)-2-(chloromethyl)-3-(oxetan-2-ylmethyl)-3H-imidazo[4,5-c]pyridine-6-carbonitrile (119 mg, 0.453 mmol) was added and the mixture was stirred at 60° C. for 2 h. The mixture was filtered and the filtrate was purified by prep-HPLC to obtain (S)-2-((4-(3-((4-chloro-2-fluorobenzyl)oxy)-4-fluorophenyl)piperazin-1-yl)methyl)-3-(oxetan-2-ylmethyl)-3H-imidazo[4,5-c]pyridine-6-carbonitrile (40 mg, yield: 15%) as a white solid. MS Calcd: 564.2; MS Found: 565.1 [M+H]⁺.

### Step D: (S)-2-((4-(3-((4-chloro-2-fluorobenzyl)oxy)-4-fluorophenyl)piperazin-1-yl)methyl)-N′-hydroxy-3-(oxetan-2-ylmethyl)-3H-imidazo[4,5-c]pyridine-6-carboximidamide

A mixture of (S)-2-((4-(3-((4-chloro-2-fluorobenzyl)oxy)-4-fluorophenyl)piperazin-1-yl)methyl)-3-(oxetan-2-ylmethyl)-3H-imidazo[4,5-c]pyridine-6-carbonitrile (40 mg, 0.07 mmol), NH₂OH·HCl (10 mg, 0.14 mmol) and DIEA (18 mg, 0.14 mmol) in EtOH (5 mL) was stirred at 90° C. for 0.5 h. The mixture was purified by prep-HPLC to obtain (S)-2-((4-(3-((4-chloro-2-fluorobenzyl)oxy)-4-fluorophenyl)piperazin-1-yl)methyl)-N′-hydroxy-3-(oxetan-2-ylmethyl)-3H-imidazo[4,5-c]pyridine-6-carboximidamide (35 mg, yield: 83%) as a white solid. MS Calcd: 597.2; MS Found: 598.0 [M+H]⁺.

### Step E: 3-{2-[(4-{3-[(4-chloro-2-fluorophenyl)methoxy]-4-fluorophenyl}piperazin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 439a)

A mixture of (S)-2-((4-(3-((4-chloro-2-fluorobenzyl)oxy)-4-fluorophenyl)piperazin-1-yl)methyl)-N′-hydroxy-3-(oxetan-2-ylmethyl)-3H-imidazo[4,5-c]pyridine-6-carboximidamide (15 mg, 0.025 mmol) and CDI (20 mg, 0.125 mmol) in THE (2 mL) was stirred at 50° C. for 16 h. The mixture was purified by prep-HPLC to obtain 3-{2-[(4-{3-[(4-chloro-2-fluorophenyl)methoxy]-4-fluorophenyl}piperazin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 439a) (7.3 mg, yield: 47%) as a white solid. MS Calcd: 623.2; MS Found: 624.1 [M+H]⁺.

¹H NMR (400 MHz, DMSO-d₆) 9.15 (s, 1H), 8.17 (s, 1H), 7.59 (t, J=8.4 Hz, 1H), 7.50 (dd, J=1.6 Hz/J=10.0 Hz, 1H), 7.35 (dd, J=2.0 Hz/J=8.4 Hz, 1H), 7.04 (dd, J=9.2 Hz/J=11.2 Hz, 1H), 6.88-6.81 (m, 1H), 6.50-6.44 (m, 1H), 5.18 (s, 2H), 5.20-5.11 (m, 1H), 4.95-4.85 (m, 1H), 4.78-4.70 (m, 1H), 4.55-4.46 (m, 1H), 4.45-4.38 (m, 1H), 4.08-3.87 (m, 2H), 3.10 (t, J=3.6 Hz, 4H), 2.78-2.58 (m, 4H), 2.05-1.95 (m, 2H). ¹⁹F-NMR (377 MHz): −114.99, −146.05.

### Example 240: 4-[(2S)-2-(4-chloro-2-fluorophenyl)-2-methyl-2H-1,3-benzodioxol-4-yl]-1-[(3-{[(2S)-oxetan-2-yl]methyl}-6-(4H-1,2,4-triazol-3-yl)-3H-imidazo[4,5-c]pyridin-2-yl)methyl]piperidine (Compound 440a)

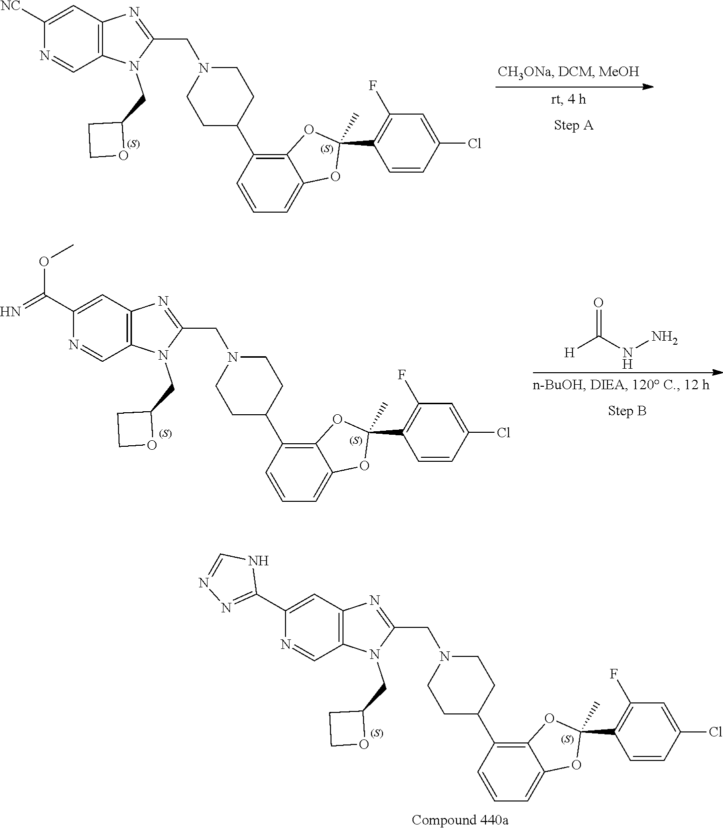

### Step A: methyl 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate

To a solution of 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (60 mg, 0.10 mmol) in DCM (1 mL) and MeOH (1 mL) was added sodium methanolate (56 mg, 1.04 mmol). The solution was stirred at room temperature for 4 hours. The reaction was quenched with water (10 mL), extracted with ethyl acetate (10 mL×2). The organic layers were combined, dried over Na₂SO₄, filtered and concentrated under vacuum. The residue was purified by Prep-TLC (DCM/MeOH=20/1) to give methyl 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (40 mg, yield: 63%) as a white solid. MS Calcd.: 605.2; MS Found: 304.0 [M/2+H]⁺.

### Step B: 4-[(2S)-2-(4-chloro-2-fluorophenyl)-2-methyl-2H-1,3-benzodioxol-4-yl]-1-[(3-{[(2S)-oxetan-2-yl]methyl}-6-(4H-1,2,4-triazol-3-yl)-3H-imidazo[4,5-c]pyridin-2-yl)methyl]piperidine (Compound 440a)

To a solution of methyl 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (50 mg, 0.082 mmol) in n-BuOH (1.5 mL) was added formohydrazide (19.8 mg, 0.330 mmol) and DIEA (42.6 mg, 0.330 mmol). The solution was stirred at 120° C. for 12 hours. The solvent was removed in vacuo. The residue was purified by Prep-HPLC to give 4-[(2S)-2-(4-chloro-2-fluorophenyl)-2-methyl-2H-1,3-benzodioxol-4-yl]-1-[(3-{[(2S)-oxetan-2-yl]methyl}-6-(4H-1,2,4-triazol-3-yl)-3H-imidazo[4,5-c]pyridin-2-yl)methyl]piperidine (Compound 440a) (5.2 mg, yield: 10%) as a white solid.

MS Calcd.: 615.2; MS Found: 616.1 [M+H]⁺.

¹H NMR (400 MHz, DMSO-d₆): δ 9.10 (s, 1H), 8.24 (s, 1H), 8.02 (brs, 1H), 7.52-7.60 (m, 2H), 7.34 (dd, J=8.4 Hz, 1.6 Hz, 1H), 6.71-6.82 (m, 3H), 5.13-5.22 (m, 1H), 4.85-4.93 (m, 1H), 4.70-4.78 (m, 1H), 4.46-4.53 (m, 1H), 4.38-4.45 (m, 1H), 4.00 (d, J=13.6 Hz, 1H), 3.84 (d, J=13.6 Hz, 1H), 3.00-3.08 (m, 1H), 2.85-2.93 (m, 1H), 2.60-2.78 (m, 2H), 2.40-2.54 (m, 1H), 2.17-2.35 (m, 2H), 2.03 (s, 3H), 1.65-1.85 (m, 4H). ¹⁹F-NMR (377 MHz): −110.79.

### Example 241: 4-[2-(4-chloro-2-fluorophenyl)-6-fluoro-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 441a)

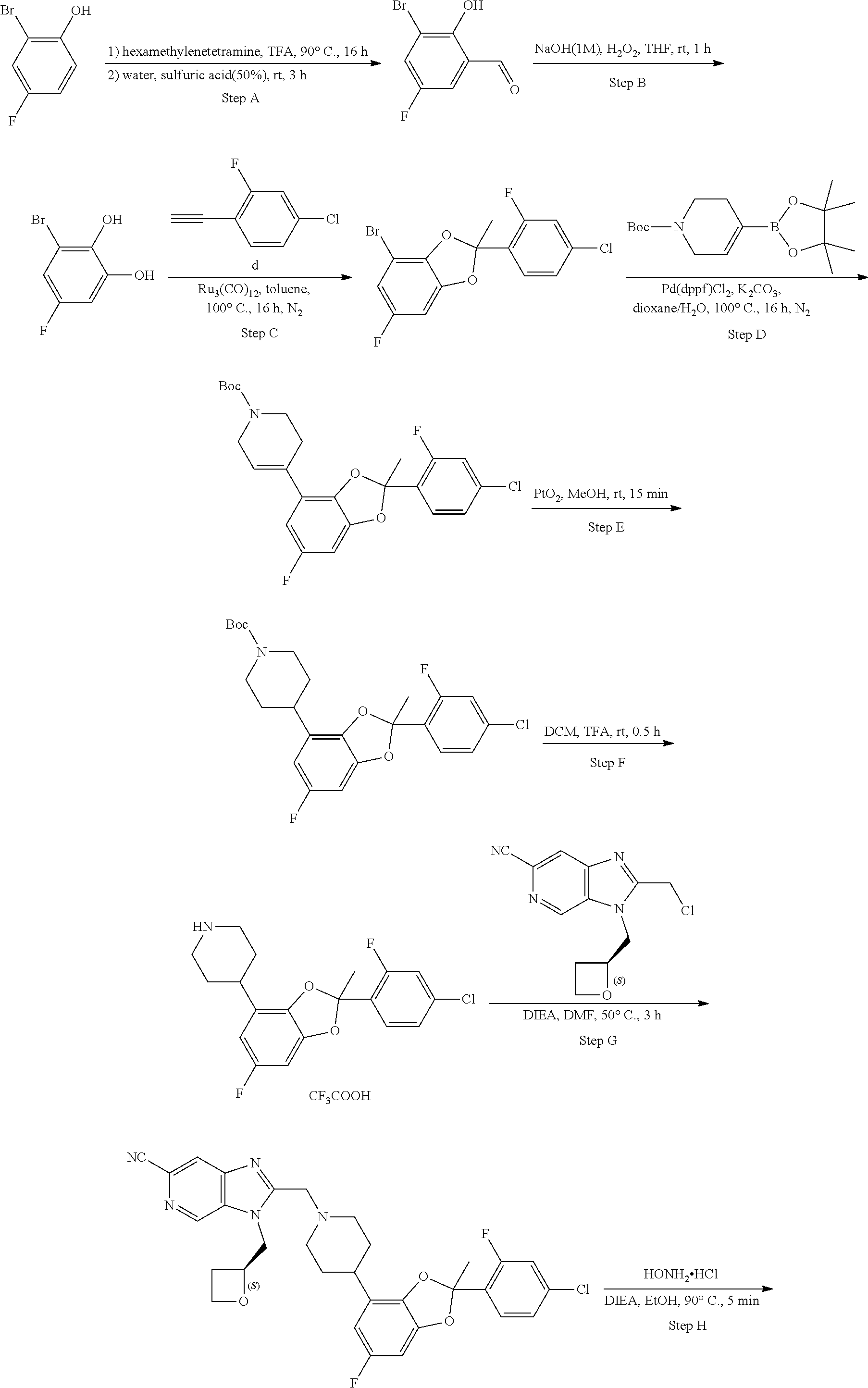

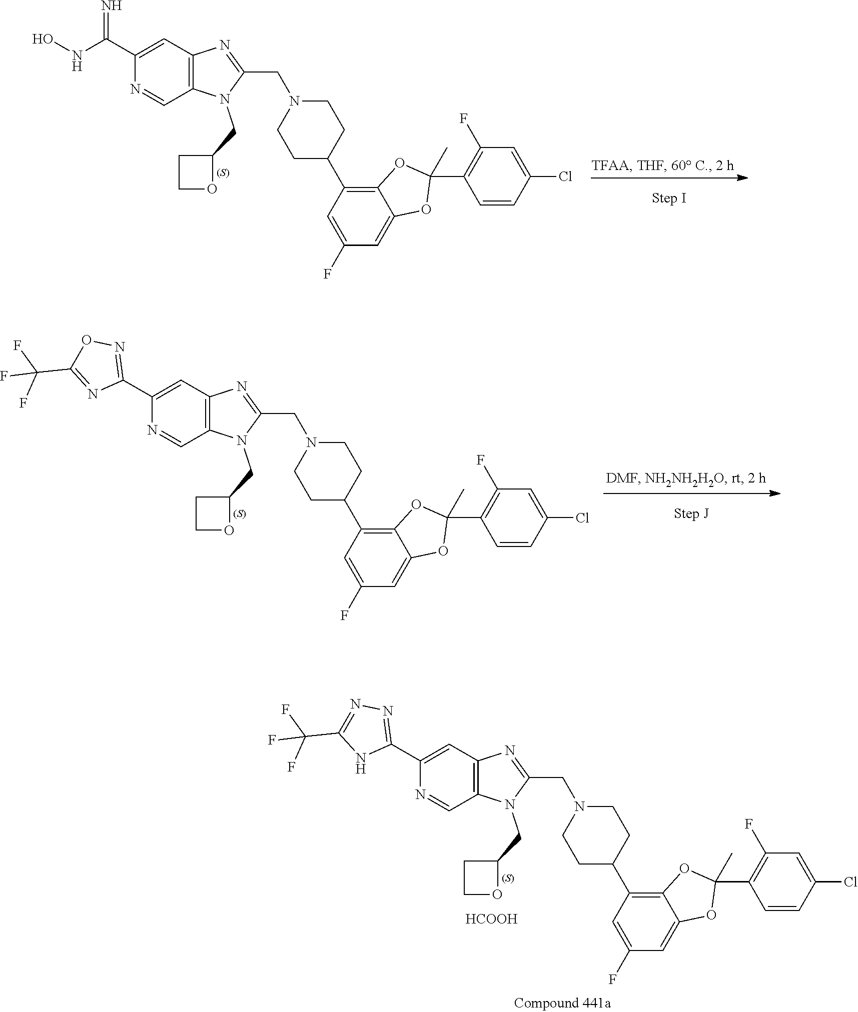

### Step A: 3-bromo-5-fluoro-2-hydroxybenzaldehyde

To a solution of 2-bromo-4-fluorophenol (5.0 g, 26.2 mmol) in TFA (13 mL) was added hexamethylenetetramine (7.3 g, 52.4 mmol). The mixture was stirred at 90° C. for 16 hours under N₂. The reaction mixture was cooled to rt and then water (50 mL) and 50% sulfuric acid (20 mL) was added. The mixture was stirred at rt for 3 hours. After the reaction was completed, the mixture was extracted with EA (100 mL). The organic phase was washed with 1N HCl (100 mL) and water (100 mL×2). The organic layer was dried over anhydrous sodium sulfate, filtered and evaporated to dryness. The organic layer was purified by column chromatography on silica gel (PE/EA=3/1) to give 3-bromo-5-fluoro-2-hydroxybenzaldehyde (3.8 g, 66% yield) as a yellow solid.

¹H NMR (400 MHz, DMSO-d₆): δ 11.02 (s, 1H), 10.10 (d, J=1.6 Hz, 1H), 7.92 (dd, J=8.0, 3.2 Hz, 1H), 7.60 (dd, J=8.0, 2.8 Hz, 1H).

### Step B: 3-bromo-5-fluorobenzene-1,2-diol

To a solution of 3-bromo-5-fluoro-2-hydroxybenzaldehyde (500 mg, 2.3 mmol) in THF (5 mL) was added NaOH (1 mol/L, 2.6 mL), then added 30% H₂O₂ (3.1 mL) slowly. The mixture was stirred at rt for 1 hour. After the reaction was completed, the pH value of the reaction mixture was adjusted to 5-6 with 1N HCl at 0° C. and diluted with water (10 mL). The mixture was extracted with EA (50 mL). The organic phase was washed with brine (20 mL×3), then dried over anhydrous sodium sulfate, filtered and evaporated to dryness. The residue was purified by column chromatography on silica gel (PE/EA=3/1) to give 3-bromo-5-fluorobenzene-1,2-diol (460 mg, 97% yield) as a dark brown solid.

¹H NMR (400 MHz, DMSO-d₆): δ 10.06 (br.s, 1H), 9.18 (br.s, 1H), 6.82 (dd, J=8.0, 2.4 Hz, 1H), 6.61 (dd, J=10.0, 2.8 Hz, 1H).

### Step C: 4-bromo-2-(4-chloro-2-fluorophenyl)-6-fluoro-2-methylbenzo[d][1,3]dioxole

To a solution of 3-bromo-5-fluorobenzene-1,2-diol (200 mg, 0.97 mmol) and 4-chloro-1-ethynyl-2-fluorobenzene (164 mg, 1.06 mmol) in toluene (5 mL) was added Ru₃(CO)₁₂ (31 mg, 0.048 mmol). The mixture was stirred at 100° C. for 16 hours under N₂. After the reaction was completed, the mixture was filtered and the filtrate evaporated to dryness. The residue was purified by column chromatography on silica gel (PE/EA=15/1) to give 4-bromo-2-(4-chloro-2-fluorophenyl)-6-fluoro-2-methylbenzo[d][1,3]dioxole (135 mg, 39% yield) as a yellow solid.

¹H NMR (400 MHz, DMSO-d₆): δ 7.55-7.62 (m, 2H), 7.38-7.42 (m, 1H), 7.01-7.08 (m, 2H), 2.10 (s, 3H).

### Step D: tert-butyl 4-(2-(4-chloro-2-fluorophenyl)-6-fluoro-2-methylbenzo[d][1,3]dioxol-4-yl)-3,6-dihydropyridine-1(2H)-carboxylate

To a mixture of 4-bromo-2-(4-chloro-2-fluorophenyl)-6-fluoro-2-methylbenzo[d][1,3]dioxole (135 mg, 0.37 mmol), tert-butyl 4-(4,4,5,5-tetramethyl-1,3,2-dioxaborolan-2-yl)-3,6-dihydropyridine-1(2H)-carboxylate (115 mg, 0.37 mmol) and K₂CO₃ (155 mg, 1.12 mmol) in dioxane/H₂O (3 mL/0.3 mL) was added Pd(dppf)Cl₂ (27 mg, 0.037 mmol). The resulting mixture was stirred at 100° C. for 16 hours under N₂. After the reaction was completed, the mixture was filtered. The filtrate was diluted with EA (50 mL), washed with brine (20 mL×3). The organic layer was dried over anhydrous sodium sulfate, filtered and evaporated to dryness. The residue was purified by column chromatography on silica gel (PE/EA=10/1) to give tert-butyl 4-(2-(4-chloro-2-fluorophenyl)-6-fluoro-2-methylbenzo[d][1,3]dioxol-4-yl)-3,6-dihydropyridine-1(2H)-carboxylate (135 mg, 78% yield) as a yellow oil. MS Calcd.: 463.1; MS Found: 486.1 [M+23]⁺.

### Step E: tert-butyl 4-(2-(4-chloro-2-fluorophenyl)-6-fluoro-2-methylbenzo[d][1,3]dioxol-4-yl)piperidine-1-carboxylate

To a solution of tert-butyl 4-(2-(4-chloro-2-fluorophenyl)-6-fluoro-2-methylbenzo[d][1,3]dioxol-4-yl)-3,6-dihydropyridine-1(2H)-carboxylate (135 mg, 0.29 mmol) in MeOH (3 mL) was added PtO₂ (10 mg). The mixture was stirred at rt for min under H₂ atmosphere. After the reaction was completed, the reaction mixture was filtered and the filtrate concentrated to give tert-butyl 4-(2-(4-chloro-2-fluorophenyl)-6-fluoro-2-methylbenzo[d][1,3]dioxol-4-yl)piperidine-1-carboxylate (120 mg, yield: 88%) as a yellow solid. MS Calcd.: 465.2; MS Found: 488.1 [M+23]⁺.

### Step F: 4-(2-(4-chloro-2-fluorophenyl)-6-fluoro-2-methylbenzo[d][1,3]dioxol-4-yl)piperidine

A mixture of tert-butyl 4-(2-(4-chloro-2-fluorophenyl)-6-fluoro-2-methylbenzo[d][1,3]dioxol-4-yl)piperidine-1-carboxylate (120 mg) in DCM/TFA (3 mL/1 mL) was stirred at rt for 30 min. After the reaction was completed, the reaction mixture was concentrated to give 4-(2-(4-chloro-2-fluorophenyl)-6-fluoro-2-methylbenzo[d][1,3]dioxol-4-yl)piperidine (120 mg, crude TFA salt) as a yellow oil. MS Calcd.: 365.1; MS Found: 366.2 [M+H]⁺.

### Step G: 2-((4-(2-(4-chloro-2-fluorophenyl)-6-fluoro-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile

To a solution of 4-(2-(4-chloro-2-fluorophenyl)-6-fluoro-2-methylbenzo[d][1,3]dioxol-4-yl)piperidine TFA salt (100 mg) in dry DMF (2 mL) was added DIEA (81 mg, 0.62 mmol). After 2 minutes, (S)-2-(chloromethyl)-3-(oxetan-2-ylmethyl)-3H-imidazo[4,5-c]pyridine-6-carbonitrile (55 mg, 0.21 mmol) was added. The mixture was stirred at 50° C. for 3 hours. After the reaction was completed, the mixture was diluted with EA (10 mL), washed with brine (10 mL×3). The organic layer was dried over anhydrous sodium sulfate, filtered and evaporated to dryness. The residue was purified by column chromatography on silica gel (PE/EA=1/1) to give 2-((4-(2-(4-chloro-2-fluorophenyl)-6-fluoro-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (80 mg, crude) as a yellow oil. MS Calcd.: 591.2; MS Found: 592.1 [M+H]⁺.

### Step H: 2-((4-(2-(4-chloro-2-fluorophenyl)-6-fluoro-2-methylbenzo[d][1,3]dioxol-4-yl)piperidin-1-yl)methyl)-N-hydroxy-3-(((S)-oxetan-2-yl)methyl)-3H-imidazo[4,5-c]pyridine-6-carboximidamide

A mixture of 2-((4-(2-(4-chloro-2-fluorophenyl)-6-fluoro-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (80 mg, 0.14 mmol), hydroxylamine hydrochloride (19 mg, 0.27 mmol) and DIEA (87 mg, 0.68 mmol) in EtOH (2 mL) was stirred at 90° C. for 5 min. After the reaction was completed, the mixture was filtered and the solid dried to give 2-((4-(2-(4-chloro-2-fluorophenyl)-6-fluoro-2-methylbenzo[d][1,3]dioxol-4-yl)piperidin-1-yl)methyl)-N-hydroxy-3-(((S)-oxetan-2-yl)methyl)-3H-imidazo[4,5-c]pyridine-6-carboximidamide (60 mg, 71.1% yield) as a white solid. MS Calcd.: 624.2; MS Found: 625.1 [M+H]⁺.

### Step I: 3-(2-((4-(2-(4-chloro-2-fluorophenyl)-6-fluoro-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole

A mixture of 2-((4-(2-(4-chloro-2-fluorophenyl)-6-fluoro-2-methylbenzo[d][1,3]dioxol-4-yl)piperidin-1-yl)methyl)-N-hydroxy-3-(((S)-oxetan-2-yl)methyl)-3H-imidazo[4,5-c]pyridine-6-carboximidamide (60 mg) and TFAA (60 mg, 0.29 mmol) in THE (2 mL) was stirred at 60° C. for 2 hours. After the reaction was completed, the mixture was diluted with EA (10 mL), washed with sat NaHCO₃ (10 mL×1) and water (10 mL×2). The organic layer was dried over anhydrous sodium sulfate, filtered and evaporated to dryness. The residue was purified by Prep-TLC (DCM/MeOH=10/1) to give 3-(2-((4-(2-(4-chloro-2-fluorophenyl)-6-fluoro-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole (40 mg, 59.3% yield) as colorless oil. MS Calcd.: 702.2; MS Found: 703.2 [M+H]⁺.

### Step J: 4-[2-(4-chloro-2-fluorophenyl)-6-fluoro-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 441a)

To a mixture of 3-(2-((4-(2-(4-chloro-2-fluorophenyl)-6-fluoro-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole (40 mg, 0.057 mmol) in DMF (2 mL) was added hydrazine hydrate (9 mg, 0.17 mmol). The mixture was stirred at room temperature for 2 hours. After the reaction was completed, the reaction mixture was purified directly by Prep-HPLC to give 4-[2-(4-chloro-2-fluorophenyl)-6-fluoro-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 441a) (5.2 mg, yield: 13%).

MS Calcd.: 701.2; MS Found: 702.3 [M+H]⁺.

¹H NMR (400 MHz, DMSO-d₆): δ 9.13 (s, 1H), 8.36 (s, 1H), 7.52-7.61 (m, 2H), 7.35 (d, J=8.4 Hz, 1H), 6.80 (dd, J=8.0, 2.4 Hz, 1H), 6.59 (dd, J=10.8, 2.4 Hz, 1H), 5.15-5.21 (m, 1H), 4.85-4.95 (m, 1H), 4.70-4.79 (m, 1H), 4.38-4.55 (m, 2H), 4.00 (d, J=13.6 Hz, 1H), 3.85 (d, J=13.6 Hz, 1H), 3.00-3.08 (m, 1H), 2.85-2.95 (m, 1H), 2.60-2.80 (m, 3H), 2.15-2.36 (m, 2H), 2.04 (s, 3H), 1.65-1.80 (m, 4H). ¹⁹F-NMR (377 MHz): −63.21, −110.74, −110.78, −119.89, −119.90.

### Example 242: 3-fluoro-4-[(2S)-4-[1-({3-[(2R)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]benzonitrile (Compound 442a)

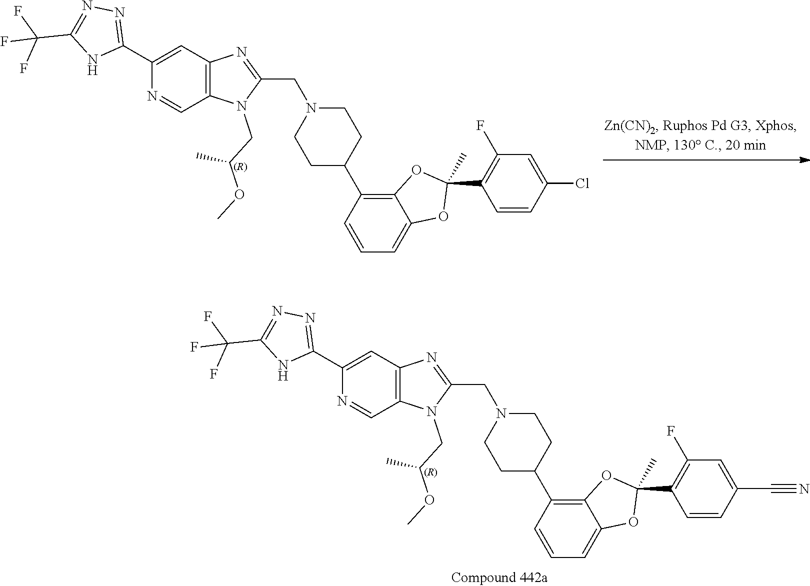

A mixture of 2-((4-((S)-2-(4-chloro-2-fluorophenyl)-2-methylbenzo[d][1,3]dioxol-4-yl)piperidin-1-yl)methyl)-3-((R)-2-methoxypropyl)-6-(5-(trifluoromethyl)-4H-1,2,4-triazol-3-yl)-3H-imidazo[4,5-c]pyridine (55 mg, 0.08 mmol), Zn(CN)₂ (28 mg, 0.24 mmol), Ruphos Pd G₃ (13.4 mg, 0.016 mmol) and Xphos (11.5 mg, 0.024 mmol) in NMP (1.5 mL) was stirred at 130° C. for 20 min under N₂. After the reaction was completed, the mixture was purified directly by Prep-HPLC (FA) to give 3-fluoro-4-[(2S)-4-[1-({3-[(2R)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]benzonitrile (Compound 442a) (14 mg, yield: 26%).

MS Calcd.: 676.2; MS Found: 677.2 [M+H]⁺.

¹H NMR (400 MHz, DMSO-d₆): δ 9.09 (d, J=0.8 Hz, 1H), 8.28 (d, J=0.8 Hz, 1H), 7.94-7.99 (m, 1H), 7.70-7.76 (m, 2H), 6.72-6.84 (m, 3H), 4.46-4.60 (m, 2H), 4.06 (d, J=14.0 Hz, 1H), 3.85-3.97 (m, 1H), 3.80 (d, J=13.6 Hz, 1H), 3.10 (s, 3H), 3.03-3.10 (m, 1H), 2.83-2.90 (m, 1H), 2.60-2.73 (m, 1H), 2.26-2.35 (m, 1H), 2.15-2.28 (m, 1H), 2.05 (s, 3H), 1.70-1.83 (m, 4H), 1.22 (d, J=6.4 Hz, 3H). ¹⁹F-NMR (377 MHz): −63.68, −110.95.

### Example 243: 3-fluoro-4-[(2S)-4-[1-({3-[(2S)-2-methoxypropyl]-6-(5-oxo-4,5-dihydro-1,2,4-oxadiazol-3-yl)-3H-imidazo[4,5-c]pyridin-2-yl}methyl)piperidin-4-yl]-2-methyl-2H-1,3-benzodioxol-2-yl]benzonitrile (Compound 443a)

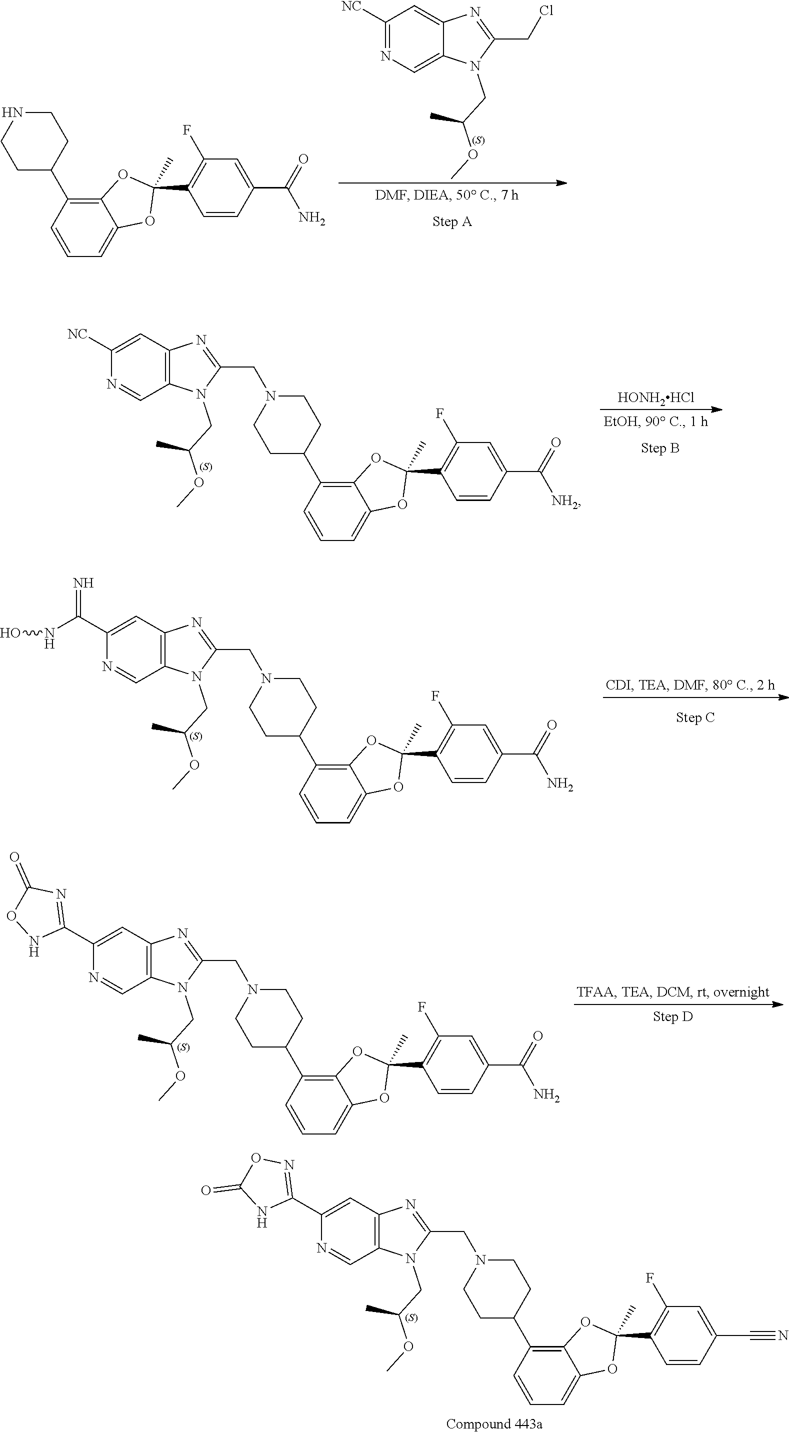

### Step A: 4-((S)-4-(1-((6-cyano-3-((S)-2-methoxypropyl)-3H-imidazo[4,5-c]pyridin-2-yl)methyl)piperidin-4-yl)-2-methylbenzo[d][1,3]dioxol-2-yl)-3-fluorobenzamide

A mixture of (S)-3-fluoro-4-(2-methyl-4-(piperidin-4-yl)benzo[d][1,3]dioxol-2-yl)benzamide TFA salt (213 mg, 0.38 mmol), (S)-2-(chloromethyl)-3-(2-methoxypropyl)-3H-imidazo[4,5-c]pyridine-6-carbonitrile (100 mg, 0.38 mmol), DIEA (147 mg, 1.14 mmol) in DMF (2 mL) was stirred at 50° C. for 7 hours. After the reaction was completed, the mixture was diluted with EA (50 mL), washed with H₂O (40 mL*3). The combined organic layer was dried over anhydrous sodium sulfate, filtered and evaporated to dryness. The residue was purified by column chromatography on silica gel (PE/EA=1/3) to give 4-((S)-4-(1-((6-cyano-3-((S)-2-methoxypropyl)-3H-imidazo[4,5-c]pyridin-2-yl)methyl)piperidin-4-yl)-2-methylbenzo[d][1,3]dioxol-2-yl)-3-fluorobenzamide (110 mg, yield: 49%) as a white solid. MS Calcd.: 584.2; MS Found: 585.2 [M+H]⁺.

### Step B: 3-fluoro-4-((S)-4-(1-((6-(N-hydroxycarbamimidoyl)-3-((S)-2-methoxypropyl)-3H-imidazo[4,5-c]pyridin-2-yl)methyl)piperidin-4-yl)-2-methylbenzo[d][1,3]dioxol-2-yl)benzamide

A solution of 4-((S)-4-(1-((6-cyano-3-((S)-2-methoxypropyl)-3H-imidazo[4,5-c]pyridin-2-yl)methyl)piperidin-4-yl)-2-methylbenzo[d][1,3]dioxol-2-yl)-3-fluorobenzamide (110 mg, 0.19 mmol), HONH₂·HCl (26 mg, 0.38 mmol), DIEA (74 mg, 0.57 mmol) in EtOH (4 mL) was stirred at 90° C. for 1 h. The reaction was filtered. The solid obtained was dried under vacuum to give 3-fluoro-4-((S)-4-(1-((6-(N-hydroxycarbamimidoyl)-3-((S)-2-methoxypropyl)-3H-imidazo[4,5-c]pyridin-2-yl)methyl)piperidin-4-yl)-2-methylbenzo[d][1,3]dioxol-2-yl)benzamide (45 mg, 40% yield) as a white solid. MS Calcd.: 617.3; MS Found: 618.2 [M+H]⁺.

### Step C: 3-fluoro-4-((S)-4-(1-((3-((S)-2-methoxypropyl)-6-(5-oxo-2,5-dihydro-1,2,4-oxadiazol-3-yl)-3H-imidazo[4,5-c]pyridin-2-yl)methyl)piperidin-4-yl)-2-methylbenzo[d][1,3]dioxol-2-yl)benzamide

A mixture of 3-fluoro-4-((S)-4-(1-((6-(N-hydroxycarbamimidoyl)-3-((S)-2-methoxypropyl)-3H-imidazo[4,5-c]pyridin-2-yl)methyl)piperidin-4-yl)-2-methylbenzo[d][1,3]dioxol-2-yl)benzamide (45 mg), CDI (18 mg, 0.11 mmol), TEA (14 mg, 0.14 mmol) in DMF (2 mL) was stirred at 80° C. for 2 hours. After the reaction was completed, the mixture was diluted with EA (30 mL), washed with H₂O (20 mL*3). The combined organic layer was dried over anhydrous sodium sulfate, filtered and concentrated to afford 3-fluoro-4-((S)-4-(1-((3-((S)-2-methoxypropyl)-6-(5-oxo-2,5-dihydro-1,2,4-oxadiazol-3-yl)-3H-imidazo[4,5-c]pyridin-2-yl)methyl)piperidin-4-yl)-2-methylbenzo[d][1,3]dioxol-2-yl)benzamide (crude, 65 mg) as yellow oil. MS Calcd.: 643.3; MS Found: 644.2 [M+H]⁺.

### Step D: 3-fluoro-4-[(2S)-4-[1-({3-[(2S)-2-methoxypropyl]-6-(5-oxo-4,5-dihydro-1,2,4-oxadiazol-3-yl)-3H-imidazo[4,5-c]pyridin-2-yl}methyl)piperidin-4-yl]-2-methyl-2H-1,3-benzodioxol-2-yl]benzonitrile (Compound 443a)

A mixture of 3-fluoro-4-((S)-4-(1-((3-((S)-2-methoxypropyl)-6-(5-oxo-2,5-dihydro-1,2,4-oxadiazol-3-yl)-3H-imidazo[4,5-c]pyridin-2-yl)methyl)piperidin-4-yl)-2-methylbenzo[d][1,3]dioxol-2-yl)benzamide (65 mg, crude), TFAA (29 mg, 0.14 mmol), TEA (21 mg, 0.21 mmol) in DCM (2 mL) was stirred at rt overnight. After the reaction was completed, the mixture was diluted with DCM (30 mL), washed with sat. NaHCO₃ (20 mL*3). The organic layer was dried over anhydrous sodium sulfate, filtered and evaporated to dryness. The residue was purified by Prep-HPLC to afford 3-fluoro-4-[(2S)-4-[1-({3-[(2S)-2-methoxypropyl]-6-(5-oxo-4,5-dihydro-1,2,4-oxadiazol-3-yl)-3H-imidazo[4,5-c]pyridin-2-yl}methyl)piperidin-4-yl]-2-methyl-2H-1,3-benzodioxol-2-yl]benzonitrile (Compound 443a) (8.2 mg, yield: 19%, two steps) as a white solid.

MS Calcd.: 625.2; MS Found: 626.4 [M+H]⁺.

¹H NMR (400 MHz, DMSO-d₆): δ 9.20 (br s, 1H), 8.24 (s, 1H), 7.99 (d, J=6.8 Hz, 1H), 7.76 (br.s, 2H), 6.72-6.90 (m, 3H), 4.43-4.68 (m, 3H), 3.70-3.90 (m, 2H), 3.48-3.52 (m, 1H), 3.16-3.22 (m, 1H), 3.11 (s, 3H), 2.51-2.67 (m, 3H), 1.70-2.16 (m, 7H), 1.23 (d, J=6.0 Hz, 3H). ¹⁹F-NMR (377 MHz): −110.89, −110.96.

### Example 244: 5-[2-({4-[(2S)-2-(4-chloro-2-fluorophenyl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4H-1,2,4-triazole-3-carbonitrile (Compound 444a)

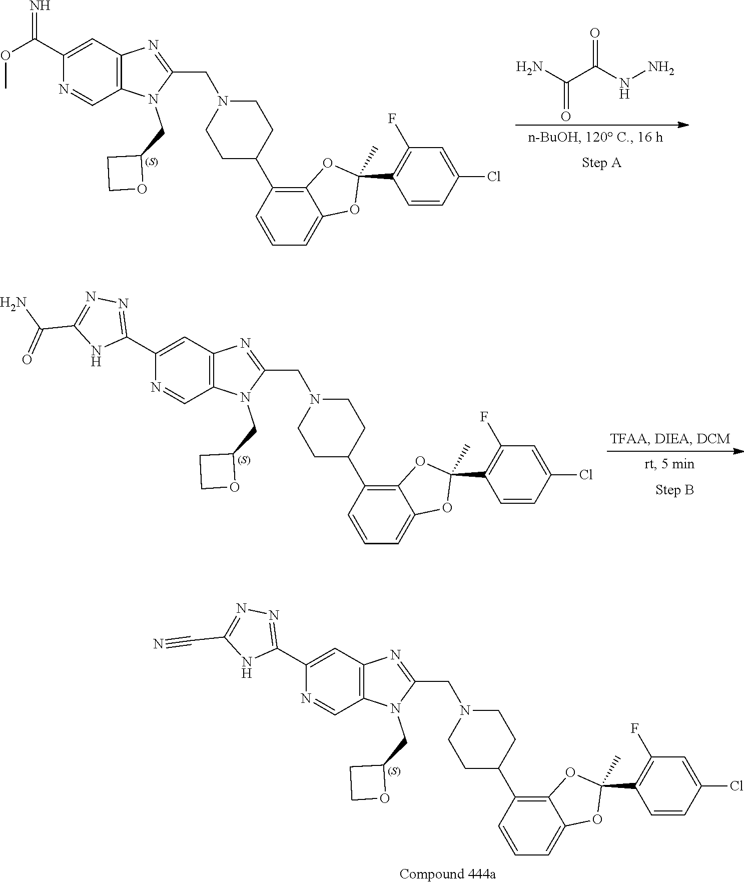

### Step A: 5-(2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-4H-1,2,4-triazole-3-carboxamide

To a solution of methyl 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (43 mg, 0.071 mmol) and 2-hydrazinyl-2-oxoacetamide (15 mg, 0.14 mmol) in n-BuOH (5 mL) was added DIEA (27 mg, 0.21 mmol) at room temperature. The reaction was stirred at 120° C. for 16 hours under N₂. After the reaction was completed, the reaction was concentrated, quenched with water (5 mL) and extracted with ethyl acetate (10 mL×3). The organic layer was combined and washed with brine (5 mL), dried over sodium sulfate, filtered and concentrated in vacuum. The residue was purified by column chromatography on silica gel (MeOH/DCM=10/1) to give 5-(2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-4H-1,2,4-triazole-3-carboxamide (30 mg, 65% yield) as a colorless oil. MS Calcd.: 658.2; MS Found: 658.9 [M+H]⁺.

### Step B: 5-[2-({4-[(2S)-2-(4-chloro-2-fluorophenyl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4H-1,2,4-triazole-3-carbonitrile (Compound 444a)

To a mixture of 5-(2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-4H-1,2,4-triazole-3-carboxamide (30 mg, 0.046 mmol), DIEA (18 mg, 0.14 mmol) in DCM (2 mL) was added TFAA (14 mg, 0.068 mmol) drop wise until the reaction was completed (rt, ˜ 5 min). After the reaction was completed, the reaction was quenched with water (5 mL) and extracted with ethyl acetate (5 mL×3). The organic layer was combined and washed with brine (5 mL), dried over sodium sulfate, filtered and concentrated in vacuum. The residue was purified by Prep-HPLC to give 5-[2-({4-[(2S)-2-(4-chloro-2-fluorophenyl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4H-1,2,4-triazole-3-carbonitrile (Compound 444a) (1.0 mg, 31% yield) as white solid.

MS Calcd.: 640.2; MS Found: 641.8 [M+H]⁺.

1H NMR (400 MHz, DMSO-d6): δ 9.18 (s, 1H), 8.32 (s, 1H), 7.52-7.60 (m, 2H), 7.34 (dd, J=8.4 Hz, J=2.0 Hz, 1H), 6.80 (d, J=4.8 Hz, 2H), 6.73-6.77 (m, 1H), 5.13-5.22 (m, 1H), 4.87-4.96 (m, 1H), 4.73-4.79 (m, 1H), 4.46-4.54 (m, 1H), 4.38-4.45 (m, 1H), 2.70 (s, 2H), 2.10-2.51 (m, 3H), 2.18 (t, J=8.0 Hz, 2H), 2.03 (s, 3H), 1.85-1.95 (m, 2H), 1.70-1.85 (m, 4H). ¹⁹F-NMR (377 MHz): −73.42, −110.75.

### Example 245: 5-chloro-2-{4-[1-({3-[(2S)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl}pyridine (Compound 445a)

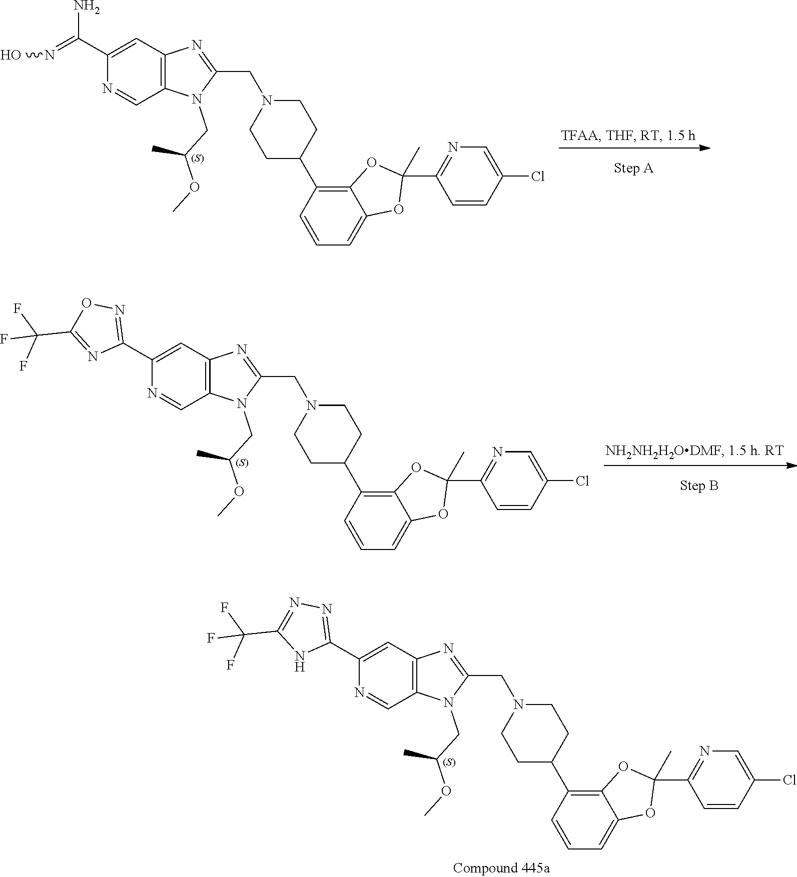

### Step A: 3-(2-((4-(2-(5-chloropyridin-2-yl)-2-methylbenzo[d][1,3]dioxol-4-yl)piperidin-1-yl)methyl)-3-((S)-2-methoxypropyl)-3H-imidazo[4,5-c]pyridin-6-yl)-5-(trifluoromethyl)-1,2,4-oxadiazole

A mixture of 2-((4-(2-(5-chloropyridin-2-yl)-2-methylbenzo[d][1,3]dioxol-4-yl)piperidin-1-yl)methyl)-N′-hydroxy-3-((S)-2-methoxypropyl)-3H-imidazo[4,5-c]pyridine-6-carboximidamide (50 mg), TFAA (54 mg, 0.25 mmol) in THF (2 mL) was stirred at room temperature for 1.5 hours. The mixture was diluted with EA (15 mL), washed with sodium carbonate solution (1M) (5 mL xl). The organic layer was dried over sodium sulfate, filtered and concentrated under reduced pressure. The residue was purified by Prep-TLC (DCM/MeOH=10/1) to afford 3-(2-((4-(2-(5-chloropyridin-2-yl)-2-methylbenzo[d][1,3]dioxol-4-yl)piperidin-1-yl)methyl)-3-((S)-2-methoxypropyl)-3H-imidazo[4,5-c]pyridin-6-yl)-5-(trifluoromethyl)-1,2,4-oxadiazole (41 mg, yield: 73%) as brown oil. MS Calcd.: 669.2; MS Found: 670.3 [M+H]⁺.

### Step B: 5-chloro-2-{4-[1-({3-[(2S)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl}pyridine (Compound 445a)

A mixture of 3-(2-((4-(2-(5-chloropyridin-2-yl)-2-methylbenzo[d][1,3]dioxol-4-yl)piperidin-1-yl)methyl)-3-((S)-2-methoxypropyl)-3H-imidazo[4,5-c]pyridin-6-yl)-5-(trifluoromethyl)-1,2,4-oxadiazole (41 mg, 0.061 mmol) and hydrazine hydrate (9.2 mg, 0.183 mmol) in DMF (3 ml) was stirred at room temperature for 1.5 hours. The reaction mixture was purified by prep-HPLC (FA) directly to give 5-chloro-2-{4-[1-({3-[(2S)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl}pyridine (Compound 445a) (8.7 mg, yield: 22%) as a white solid.

MS Calcd.: 668.2; MS Found: 669.2 [M+H]⁺.

¹H NMR (400 MHz, DMSO-d6): δ 9.06 (d, J=1.6 Hz, 1H), 8.71 (dd, J=5.6 Hz, 2.0 Hz, 1H), 8.27 (s, 1H), 7.97-8.03 (m, 1H), 7.59 (dd, J=8.4 Hz, 6.4 Hz, 1H), 6.70-6.82 (m, 3H), 4.40-4.57 (m, 2H), 4.06 (dd, J=13.6 Hz, 1.6 Hz, 1H), 3.83-3.95 (m, 1H), 3.76 (dd, J=13.6 Hz, 4.0 Hz, 1H), 3.03-3.12 (m, 4H), 2.84 (d, 1H), 2.60-2.70 (m, 1H), 2.22-2.35 (m, 1H), 2.12-2.22 (m, 1H), 2.00 (m, 3H), 1.68-1.85 (m, 4H), 1.18-1.22 (m, 3H). ¹⁹F-NMR (377 MHz): −63.53.

### Example 246: 1-{[2-({4-[2-(4-chlorophenyl)-2-methyl-2H-1,3-benzodioxol-4-yl]piperidin-1-yl}methyl)-5-[5-(trifluoromethyl)-4H-1,2,4-triazol-3-yl]-1H-1,3-benzodiazol-1-yl]methyl}cyclopropane-1-carbonitrile (Compound 446)

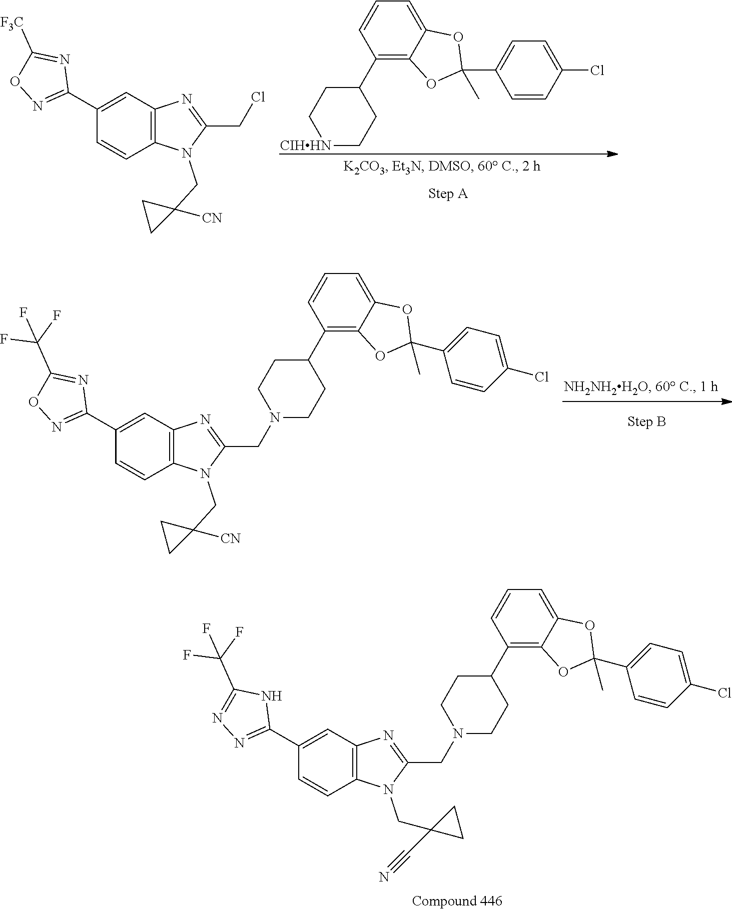

### Step A: 1-((2-((4-(2-(4-chlorophenyl)-2-methylbenzo[d][1,3]dioxol-4-yl)piperidin-1-yl)methyl)-5-(5-(trifluoromethyl)-1,2,4-oxadiazol-3-yl)-1H-benzo[d]imidazol-1-yl)methyl)cyclopropane-1-carbonitrile

A mixture of 4-(2-(4-chlorophenyl)-2-methylbenzo[d][1,3]dioxol-4-yl)piperidine hydrochloride (43 mg, 0.13 mmol) and K₂CO₃ (54 mg, 0.39 mmol), Et₃N (66 mg, 0.65 mmol) in DMSO (6 mL) was stirred at 25° C. for 0.5 hour. Then 1-((2-(chloromethyl)-5-(5-(trifluoromethyl)-1,2,4-oxadiazol-3-yl)-1H-benzo[d]imidazol-1-yl)methyl)cyclopropane-1-carbonitrile (50 mg, 0.13 mmol) was added to the mixture was stirred at 60° C. for 2 hours. The mixture was poured into water (30 mL) and extracted with EA (3×30 mL), the combined organic layer was washed with brine, dried over sodium sulfate, filtered and concentrated under reduced pressure. The residue was purified by pre-TLC (PE:EA=1:1) to give 1-((2-((4-(2-(4-chlorophenyl)-2-methylbenzo[d][1,3]dioxol-4-yl)piperidin-1-yl)methyl)-5-(5-(trifluoromethyl)-1,2,4-oxadiazol-3-yl)-1H-benzo[d]imidazol-1-yl)methyl)cyclopropane-1-carbonitrile (20 mg, yield: 22%) as a colorless oil. MS Calcd.: 674.2; MS Found: 675.4 [M+H]⁺.

### Step B: 1-{[2-({4-[2-(4-chlorophenyl)-2-methyl-2H-1,3-benzodioxol-4-yl]piperidin-1-yl}methyl)-5-[5-(trifluoromethyl)-4H-1,2,4-triazol-3-yl]-1H-1,3-benzodiazol-1-yl]methyl}cyclopropane-1-carbonitrile (Compound 446)

A mixture of 1-((2-((4-(2-(4-chlorophenyl)-2-methylbenzo[d][1,3]dioxol-4-yl)piperidin-1-yl)methyl)-5-(5-(trifluoromethyl)-1,2,4-oxadiazol-3-yl)-1H-benzo[d]imidazol-1-yl)methyl)cyclopropane-1-carbonitrile (20 mg, 0.03 mmol) and NH₂NH₂·H₂O (0.5 mL) in EtOH (2 mL) was stirred at 60° C. for 1 hour under N₂. The reaction mixture was purified by prep-HPLC to give 1-{[2-({4-[2-(4-chlorophenyl)-2-methyl-2H-1,3-benzodioxol-4-yl]piperidin-1-yl}methyl)-5-[5-(trifluoromethyl)-4H-1,2,4-triazol-3-yl]-1H-1,3-benzodiazol-1-yl]methyl}cyclopropane-1-carbonitrile (Compound 446) (4.8 mg, yield: 24%) as white solid.

MS Calcd.: 673.2; MS Found: 674.1 [M+H]⁺.

¹H NMR (400 MHz, MeOD): δ 8.29 (s, 1H), 8.23 (s, 1H), 7.94 (dd, J=10.0 Hz, 1.6 Hz, 1H), 7.77 (d, J=12.8 Hz, 1H), 7.51-7.45 (m, 2H), 7.32-7.26 (m, J=13.6 Hz, 2H), 6.69-6.55 (m, 3H), 4.73-4.68 (m, 2H), 3.94 (s, 2H), 3.00-2.93 (m, 2H), 2.70-2.59 (m, 1H), 2.24 (t, J=10.80 Hz, 2H), 1.95-1.78 (m, 5H), 1.78-1.67 (m, 2H), 1.37 (s, 4H). (FA salt)¹⁹F-NMR (377 MHz): −66.75.

### Example 247: 3-fluoro-4-[(2-fluoro-5-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-1,2,3,6-tetrahydropyridin-4-yl}phenoxy)methyl]benzonitrile (Compound 300a)

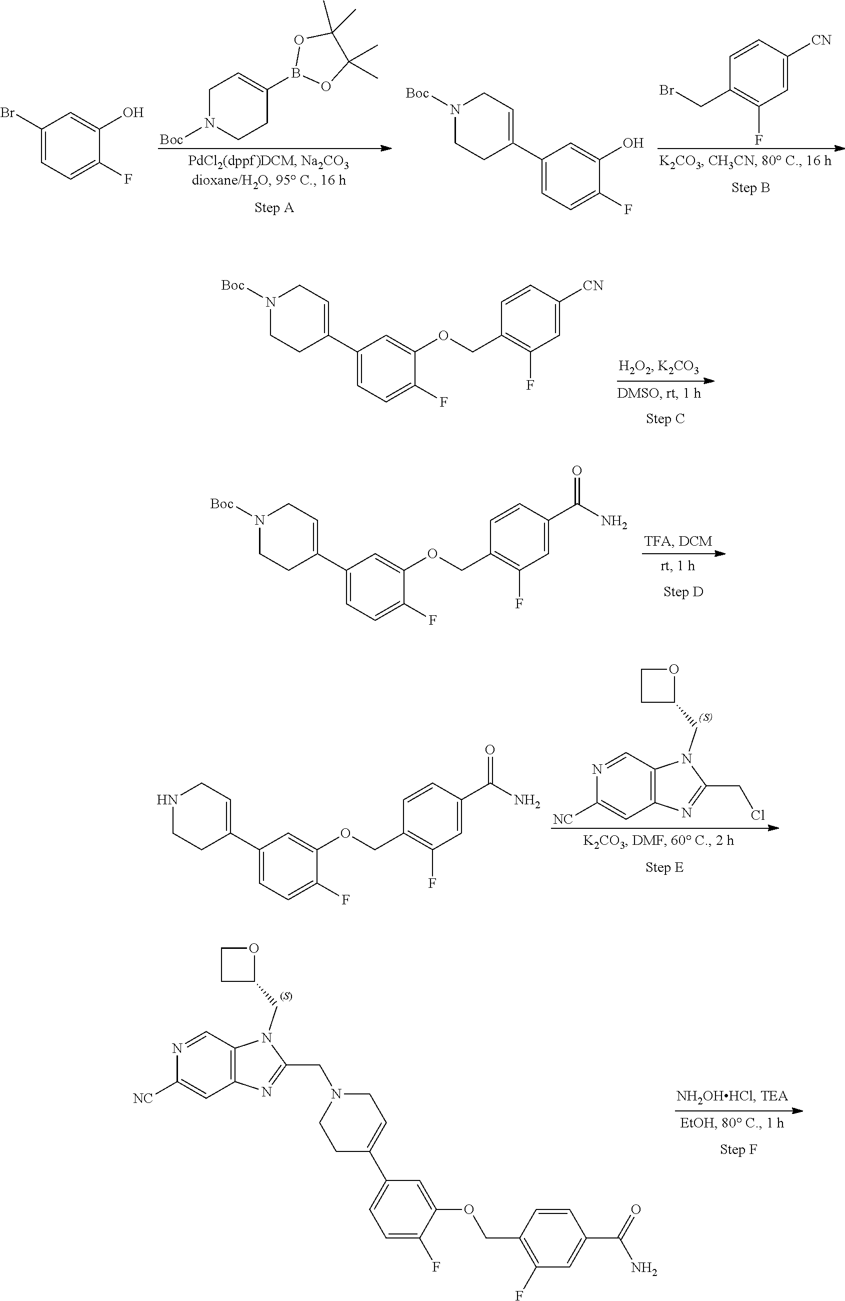

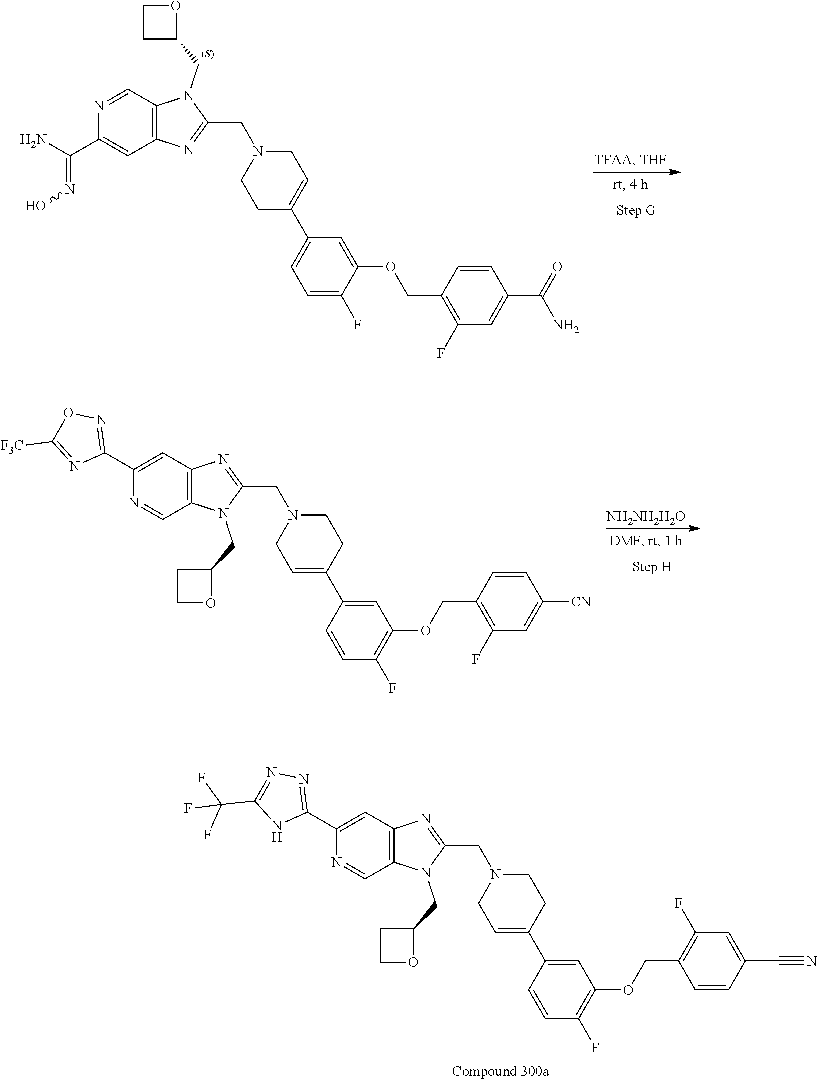

### Step A: tert-butyl 4-(4-fluoro-3-hydroxyphenyl)-3,6-dihydropyridine-1(2H)-carboxylate

A mixture of 5-bromo-2-fluorophenol (5.0 g, 26.17 mmol), tert-butyl 4-(4,4,5,5-tetramethyl-1,3,2-dioxaborolan-2-yl)-3,6-dihydropyridine-1(2H)-carboxylate (12.1 g, 39.3 mmol), PdCl2(dppf)DCM (2.1 g, 2.6 mmol) and Na₂CO₃ (8.3 g, 78.5 mmol) in dioxane (40.0 mL)/H₂O (4.0 mL) was stirred at 95° C. for 16 hours. After the reaction was completed, the reaction was quenched with water (60.0 mL) and extracted with ethyl acetate (100 mL×3). The organic layer was combined and washed with brine (40 mL×2), dried over sodium sulfate, filtered. The filtrate was concentrated in vacuum. The residue was purified by column chromatography on silica gel (PE:EA=1:9) to give tert-butyl 4-(4-fluoro-3-hydroxyphenyl)-3,6-dihydropyridine-1(2H)-carboxylate (6.0 g, yield: 78%) as a white solid. MS Calcd.: 293.1; MS Found: 238.1 [M-56+H]⁺.

### Step B: tert-butyl 4-(3-((4-cyano-2-fluorobenzyl)oxy)-4-fluorophenyl)-3,6-dihydropyridine-1(2H)-carboxylate

A mixture of tert-butyl 4-(4-fluoro-3-hydroxyphenyl)-3,6-dihydropyridine-1(2H)-carboxylate (2.0 g, 6.8 mmol), 4-(bromomethyl)-3-fluorobenzonitrile (2.6 g, 17.1 mmol) and K₂CO₃ (2.8 g, 20.5 mmol) in CH₃CN (30.0 mL) was stirred at 80° C. for 16 hours. After the reaction was completed, the reaction was filtered. The filtrate was concentrated in vacuum. The residue was purified by column chromatography on silica gel (PE:EA=10:1) to give tert-butyl 4-(3-((4-cyano-2-fluorobenzyl)oxy)-4-fluorophenyl)-3,6-dihydropyridine-1(2H)-carboxylate (2.01 g, yield: 69%) as a white solid. MS Calcd.: 426.2; MS Found: 249.1 [M+23]⁺.

### Step C: tert-butyl 4-(3-((4-carbamoyl-2-fluorobenzyl)oxy)-4-fluorophenyl)-3,6-dihydropyridine-1(2H)-carboxylate

To a mixture of tert-butyl 4-(3-((4-cyano-2-fluorobenzyl)oxy)-4-fluorophenyl)-3,6-dihydropyridine-1(2H)-carboxylate (600 mg, 1.41 mmol) and K₂CO₃ (584 mg, 4.23 mmol) in DMSO (10.0 mL) was added H₂O₂ (1.5 mL). The reaction mixture was stirred at room temperature for 1 hour. After the reaction was completed, the reaction was quenched with water (20.0 mL) and extracted with ethyl acetate (40 mL×3). The organic layer was combined and washed with brine (40 mL×2), dried over sodium sulfate, filtered. The filtrate was concentrated in vacuum. The residue was purified by column chromatography on silica gel (PE:EA=4:1) to give tert-butyl 4-(3-((4-carbamoyl-2-fluorobenzyl)oxy)-4-fluorophenyl)-3,6-dihydropyridine-1(2H)-carboxylate (400 mg, yield: 64%) as a white solid. MS Calcd.: 444.2; MS Found: 467.6 [M+Na]⁺.

### Step D: 3-fluoro-4-((2-fluoro-5-(1,2,3,6-tetrahydropyridin-4-yl)phenoxy)methyl)benzamide

To a mixture of tert-butyl 4-(3-((4-carbamoyl-2-fluorobenzyl)oxy)-4-fluorophenyl)-3,6-dihydropyridine-1(2H)-carboxylate (400 mg, 0.90 mmol) in DCM (8.0 mL) was added TFA (2.0 mL). The reaction mixture was stirred at room temperature for 1 hour. After the reaction was completed, the reaction was adjusted to pH=7-8 with saturated sodium bicarbonate and extracted with ethyl acetate (40 mL×3). The organic layer was dried over sodium sulfate, filtered. The filtrate was concentrated to give 3-fluoro-4-((2-fluoro-5-(1,2,3,6-tetrahydropyridin-4-yl)phenoxy)methyl)benzamide (170 mg, yield: 55%) as yellow oil. MS Calcd.: 344.1; MS Found: 345.6 [M+H]⁺.

### Step E: (S)-4-((5-(1-((6-cyano-3-(oxetan-2-ylmethyl)-3H-imidazo[4,5-c]pyridin-2-yl)methyl)-1,2,3,6-tetrahydropyridin-4-yl)-2-fluorophenoxy)methyl)-3-fluorobenzamide

A mixture of 3-fluoro-4-((2-fluoro-5-(1,2,3,6-tetrahydropyridin-4-yl)phenoxy)methyl)benzamide (170 mg, 0.49 mmol), (S)-2-(chloromethyl)-3-(oxetan-2-ylmethyl)-3H-imidazo[4,5-c]pyridine-6-carbonitrile (100 mg, 0.38 mmol) and K₂CO₃ (105 mg, 0.76 mmol) in DMF (3.0 mL) was stirred at 60° C. for 2 hours. After the reaction was completed, the reaction mixture was filtered and the filtrate was concentrated in vacuum. The residue was purified by column chromatography on silica gel (DCM:MeOH=30:1) to give (S)-4-((5-(1-((6-cyano-3-(oxetan-2-ylmethyl)-3H-imidazo[4,5-c]pyridin-2-yl)methyl)-1,2,3,6-tetrahydropyridin-4-yl)-2-fluorophenoxy)methyl)-3-fluorobenzamide (160 mg, yield: 74%) as a white solid. MS Calcd.: 570.2; MS Found: 571.1 [M+H]⁺.

### Step F: (S)-3-fluoro-4-((2-fluoro-5-(1-((6-(N′-hydroxycarbamimidoyl)-3-(oxetan-2-ylmethyl)-3H-imidazo[4,5-c]pyridin-2-yl)methyl)-1,2,3,6-tetrahydropyridin-4-yl)phenoxy)methyl) benzamide

To a solution of (S)-4-((5-(1-((6-cyano-3-(oxetan-2-ylmethyl)-3H-imidazo[4,5-c]pyridin-2-yl)methyl)-1,2,3,6-tetrahydropyridin-4-yl)-2-fluorophenoxy)methyl)-3-fluorobenzamide (160 mg, 0.28 mmol) and TEA (142 mg, 1.40 mmol) in EtOH (4.0 mL) was added HONH₂·HCl (59 mg, 0.84 mmol). The reaction was stirred at 80° C. for 1 hour. After the reaction was completed, the reaction mixture was filtered. The solid obtained was dried to give (S)-3-fluoro-4-((2-fluoro-5-(1-((6-(N′-hydroxycarbamimidoyl)-3-(oxetan-2-ylmethyl)-3H-imidazo[4,5-c]pyridin-2-yl)methyl)-1,2,3,6-tetrahydropyridin-4-yl)phenoxy) methyl)benzamide (140 mg, yield: 83%) as a white solid. MS Calcd.: 603.2; MS Found: 604.2 [M+H]⁺.

### Step G: (S)-3-fluoro-4-((2-fluoro-5-(1-((3-(oxetan-2-ylmethyl)-6-(5-(trifluoromethyl)-1,2,4-oxadiazol-3-yl)-3H-imidazo[4,5-c]pyridin-2-yl)methyl)-1,2,3,6-tetrahydropyridin-4-yl)phenoxy)methyl)benzonitrile

To a mixture of (S)-3-fluoro-4-((2-fluoro-5-(1-((6-(N′-hydroxycarbamimidoyl)-3-(oxetan-2-ylmethyl)-3H-imidazo[4,5-c]pyridin-2-yl)methyl)-1,2,3,6-tetrahydropyridin-4-yl)phenoxy)methyl)benzamide (120 mg) in THE (3.0 mL) was added TFAA (209 mg, 1.00 mmol) and stirred at rt for 4 hours. After the reaction was completed, the reaction was quenched with saturated sodium bicarbonate (10.0 mL) and extracted with ethyl acetate (30 mL×3). The organic layer was dried over sodium sulfate, filtered and the filtrate was concentrated in vacuum. The residue was purified by column chromatography on silica gel (PE:EA=2:1) to give (S)-3-fluoro-4-((2-fluoro-5-(1-((3-(oxetan-2-ylmethyl)-6-(5-(trifluoromethyl)-1,2,4-oxadiazol-3-yl)-3H-imidazo[4,5-c]pyridin-2-yl)methyl)-1,2,3,6-tetrahydropyridin-4-yl)phenoxy)methyl)benzonitrile (80 mg, yield: 60%) as a yellow solid. MS Calcd.: 663.2; MS Found: 664.2 [M+H]⁺.

### Step H: 3-fluoro-4-[(2-fluoro-5-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-1,2,3,6-tetrahydropyridin-4-yl}phenoxy)methyl]benzonitrile (Compound 300a)

To a solution of (S)-3-fluoro-4-((2-fluoro-5-(1-((3-(oxetan-2-ylmethyl)-6-(5-(trifluoromethyl)-1,2,4-oxadiazol-3-yl)-3H-imidazo[4,5-c]pyridin-2-yl)methyl)-1,2,3,6-tetrahydropyridin-4-yl)phenoxy)methyl)benzonitrile (80 mg, 0.12 mmol) in DMF (2.0 mL) was added NH₂NH₂·H₂O (23 mg, 0.48 mmol). The reaction mixture was stirred at room temperature for 1 hour. After the reaction was completed, the mixture was purified directly by Prep-HPLC (0.1% FA/H₂O/CH₃CN) to give 3-fluoro-4-[(2-fluoro-5-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-1,2,3,6-tetrahydropyridin-4-yl}phenoxy)methyl]benzonitrile (Compound 300a) (38 mg, yield: 48%) as a white solid.

MS Calcd.: 662.2; MS Found: 663.0 [M+H]⁺.

¹H NMR (400 MHz, DMSO-d₆): δ 9.17 (s, 1H), 8.30 (s, 1H), 7.92 (d, J=10.0 Hz, 1H), 7.76 (d, J=3.6 Hz, 2H), 7.33 (dd, J=8.0 Hz, 1.6 Hz, 1H), 7.16-7.22 (m, 1H), 7.00-7.06 (m, 1H), 6.14-6.20 (m, 1H), 5.35 (s, 2H), 5.08-5.17 (m, 1H), 4.88-4.97 (m, 1H), 4.72-4.80 (m, 1H), 4.45-4.53 (m, 1H), 4.37-4.44 (m, 1H), 4.12 (d, J=14.0 Hz, 1H), 3.99 (d, J=13.6 Hz, 1H), 3.13-3.30 (m, 2H), 2.65-2.83 (m, 3H), 2.35-2.52 (m, 3H). ¹⁹F-NMR (377 MHz): −63.70, −115.10, −136.52.

### Example 248: 3-[6-({4-[(2S)-2-(4-chloro-2-fluorophenyl)-2-methyl-2H-1,3-benzodioxol-4-yl]piperidin-1-yl}methyl)-7-{[(2S)-oxetan-2-yl]methyl}-7H-imidazo[4,5-c]pyridazin-3-yl]-4,5-dihydro-1,2,4-oxadiazol-5-one (Compound 348a)

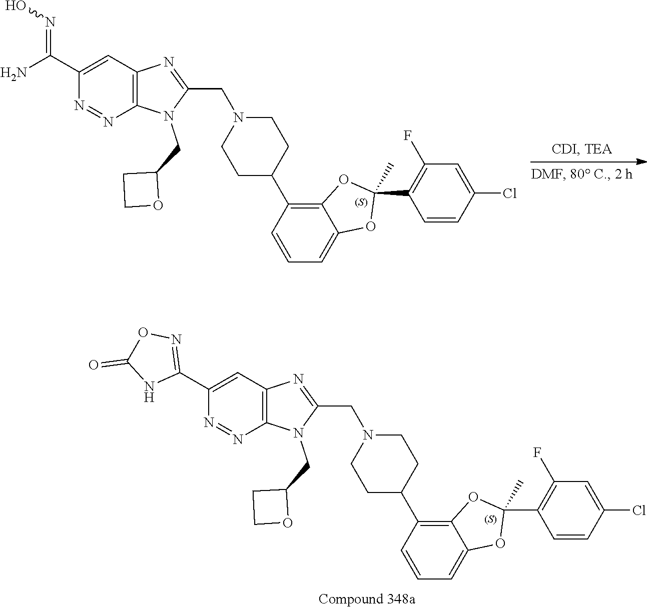

A mixture of 6-((4-((S)-2-(4-chloro-2-fluorophenyl)-2-methylbenzo[d][1,3]dioxol-4-yl)piperidin-1-yl)methyl)-N′-hydroxy-7-(((S)-oxetan-2-yl)methyl)-7H-imidazo[4,5-c]pyridazine-3-carboximidamide (40 mg), CDI (21 mg, 0.13 mmol) and TEA (27 mg, 0.26 mmol) in DMF (2.0 mL) was stirred at 80° C. for 2 hours. After the reaction was completed, the mixture was purified directly by Prep-HPLC (0.1% FA/H₂O/CH₃CN) to give 3-[6-({4-[(2S)-2-(4-chloro-2-fluorophenyl)-2-methyl-2H-1,3-benzodioxol-4-yl]piperidin-1-yl}methyl)-7-{[(2S)-oxetan-2-yl]methyl}-7H-imidazo[4,5-c]pyridazin-3-yl]-4,5-dihydro-1,2,4-oxadiazol-5-one (Compound 348a) (7.3 mg, yield: 17%) as a white solid.

MS Calcd.: 633.2; MS Found: 634.2 [M+H]⁺.

¹H NMR (400 MHz, DMSO-d6): δ 8.42 (s, 1H), 7.54-7.61 (m, 2H), 7.34 (dd, J=8.4 Hz, 2.0 Hz, 1H), 6.73-6.82 (m, 3H), 5.23-5.32 (m, 1H), 4.99-5.07 (m, 1H), 4.85-4.93 (m, 1H), 4.38-4.54 (m, 2H), 4.00-4.13 (m, 2H), 2.95-3.07 (m, 2H), 2.63-2.80 (m, 2H), 2.51-2.60 (m, 1H), 2.26-2.37 (m, 2H), 2.03 (s, 3H), 1.70-1.88 (m, 4H). ¹⁹F-NMR (377 MHz): −110.78.

### Example 249: 4-[(2S)-2-(4-chloro-2-fluorophenyl)-2-methyl-2H-1,3-benzodioxol-4-yl]-1-{[6-(5-ethyl-4H-1,2,4-triazol-3-yl)-3-{[(2S)-oxetan-2-yl]methyl}-3H-imidazo[4,5-b]pyridin-2-yl]methyl}piperidine (Compound 352a)

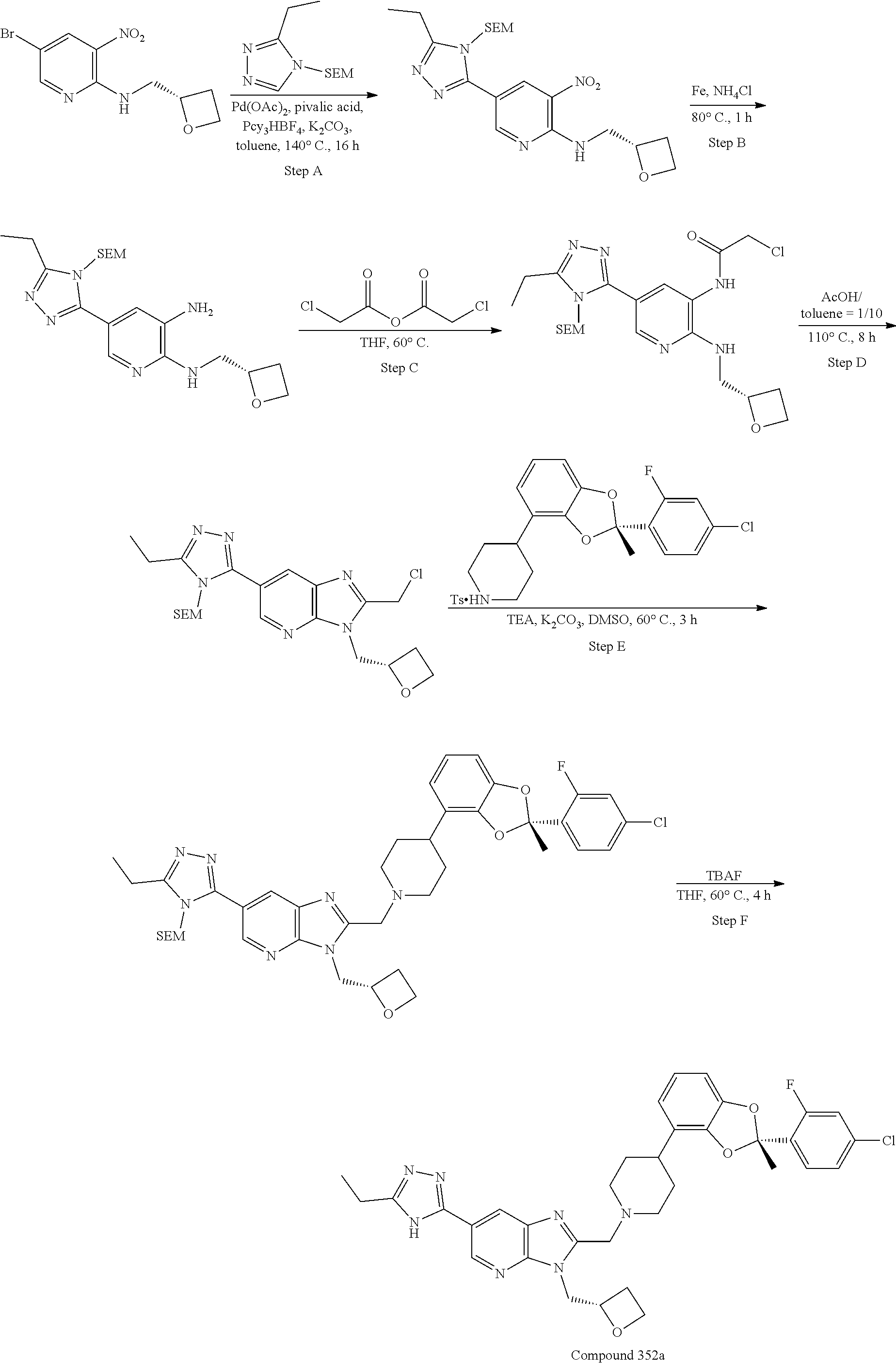

### Step A: (S)-5-(5-ethyl4-(2-(trimethylsilyl)ethoxy)methyl)-4H-1,2,4-triazol-3-yl)-3-nitro-N-(oxetan-2-ylmethyl)pyridin-2-amine

A mixture of (S)-5-bromo-3-nitro-N-(oxetan-2-ylmethyl)pyridin-2-amine (110 mg, 0.38 mmol), 3-ethyl-4-((2-(trimethylsilyl)ethoxy)methyl)-4H-1,2,4-triazole (0.26 g, 1.15 mmol) (this compound was prepared using the similar procedure as shown in the step C in preparation of Compound 283a, similar case of regio-isomers were observed), Pd(OAc)₂ (10.0 mg, 0.038 mmol), pivalic acid (26 mg, 0.25 mmol), Pcy₃·HBF₄ (30 mg, 0.08 mmol) and K₂CO₃ (315 mg, 2.28 mmol) in toluene (7 mL) was stirred at 140° C. under Ar for 16 hours. Upon cooling down, the mixture was concentrated under vacuum, purified by Prep-TLC (PE/EA=2/1) to furnish (S)-5-(5-ethyl-4-((2-(trimethylsilyl)ethoxy)methyl)-4H-1,2,4-triazol-3-yl)-3-nitro-N-(oxetan-2-ylmethyl)pyridin-2-amine (165 mg crude, yield: ˜100%) as a yellow oil. MS Calcd.: 434.2; MS Found: 435.2 [M+H]⁺.

### Step B: (S)-5-(5-ethyl-4-((2-(trimethylsilyl)ethoxy)methyl)-4H-1,2,4-triazol-3-yl)-N2-(oxetan-2-ylmethyl)pyridine-2,3-diamine

A mixture of (S)-5-(5-ethyl-4-((2-(trimethylsilyl)ethoxy)methyl)-4H-1,2,4-triazol-3-yl)-3-nitro-N-(oxetan-2-ylmethyl)pyridin-2-amine (165 mg, 0.38 mmol) and NH₄Cl (210 mg, 3.80 mmol) in EtOH/H₂O (7 mL/1.5 mL) was stirred at 80° C. for 10 min. Fe (110 mg, 1.90 mmol) was added, the mixture was stirred at 80° C. for 1 hr. Upon cooling down, the mixture was diluted with DCM/MeOH (V/V=10/1), washed with brine, dried over Na₂SO₄, concentrated under vacuum to furnish (S)-5-(5-ethyl-4-((2-(trimethylsilyl)ethoxy)methyl)-4H-1,2,4-triazol-3-yl)-N2-(oxetan-2-ylmethyl)pyridine-2,3-diamine (148 mg crude) as a yellow oil. MS Calcd.: 404.2; MS Found: 405.2 [M+H]⁺.

### Step C: (S)-2-chloro-N-(5-(5-ethyl-4-((2-(trimethylsilyl)ethoxy)methyl)-4H-1,2,4-triazol-3-yl)-2-((oxetan-2-ylmethyl)amino)pyridin-3-yl)acetamide

To a solution of (S)-5-(5-ethyl-4-((2-(trimethylsilyl)ethoxy)methyl)-4H-1,2,4-triazol-3-yl)-N2-(oxetan-2-ylmethyl)pyridine-2,3-diamine (148 mg) in THE (10 mL) was added 2-chloroacetic anhydride (44 mg, 0.25 mmol) at 0° C., the mixture was stirred at 60° C. under Ar for 16 hours. The mixture was diluted with EA, washed with aq NaHCO₃ and brine, dried over Na₂SO₄, concentrated to dryness and purified by pre-TLC to furnish (S)-2-chloro-N-(5-(5-ethyl-4-((2-(trimethylsilyl)ethoxy)methyl)-4H-1,2,4-triazol-3-yl)-2-((oxetan-2-ylmethyl)amino)pyridin-3-yl)acetamide (20 mg pure, yield: 12%) as a brown oil. MS Calcd.: 480.2; MS Found: 481.2 [M+H]⁺.

### Step D: (S)-2-(chloromethyl)-6-(5-ethyl-4-((2-(trimethylsilyl)ethoxy)methyl)-4H-1,2,4-triazol-3-yl)-3-(oxetan-2-ylmethyl)-3H-imidazo[4,5-b]pyridine

To a solution of (S)-2-chloro-N-(5-(5-ethyl-4-((2-(trimethylsilyl)ethoxy)methyl)-4H-1,2,4-triazol-3-yl)-2-((oxetan-2-ylmethyl)amino)pyridin-3-yl)acetamide (20 mg, 0.042 mmol) in toluene (2 mL) was added AcOH (0.2 mL), the mixture was stirred at 110° C. for 8 hours. The mixture was diluted with EA, washed with aq NaHCO₃ and brine, dried over Na₂SO₄, concentrated to furnish (S)-2-(chloromethyl)-6-(5-ethyl-4-((2-(trimethylsilyl)ethoxy)methyl)-4H-1,2,4-triazol-3-yl)-3-(oxetan-2-ylmethyl)-3H-imidazo[4,5-b]pyridine (20 mg crude) as a yellow oil. MS Calcd.: 462.2; MS Found: 463.2 [M+H]⁺.

### Step E: 2-((4-((S)-2-(4-chloro-2-fluorophenyl)-2-methylbenzo[d][1,3]dioxol-4-yl)piperidin-1-yl)methyl)-6-(5-ethyl-4-((2-(trimethylsilyl)ethoxy)methyl)-4H-1,2,4-triazol-3-yl)-3-(((S)-oxetan-2-yl)methyl)-3H-imidazo[4,5-b]pyridine

A mixture of (S)-4-(2-(4-chloro-2-fluorophenyl)-2-methylbenzo[d][1,3]dioxol-4-yl)-1-tosyl-1l4-piperidine (20 mg, 0.043 mmol), TEA (22 mg, 0.21 mmol) and K₂CO₃ (18 mg, 0.13 mmol) in DMSO (2 mL) was stirred at rt for 10 mins. (S)-2-(chloromethyl)-6-(5-ethyl-4-((2-(trimethylsilyl)ethoxy)methyl)-4H-1,2,4-triazol-3-yl)-3-(oxetan-2-ylmethyl)-3H-imidazo[4,5-b]pyridine (20 mg, 0.043 mmol) in DMSO (3 mL) was added, the mixture was stirred at 60° C. for 3 hrs. The reaction was diluted with EA, washed with H₂O and brine, dried over sodium sulfate, concentrated in vacuum, purified by prep-TLC (DCM/MeOH=20/1) to give 2-((4-((S)-2-(4-chloro-2-fluorophenyl)-2-methylbenzo[d][1,3]dioxol-4-yl)piperidin-1-yl)methyl)-6-(5-ethyl-4-((2-(trimethylsilyl)ethoxy)methyl)-4H-1,2,4-triazol-3-yl)-3-(((S)-oxetan-2-yl)methyl)-3H-imidazo[4,5-b]pyridine (10 mg, yield: 30%) as a yellow oil. MS Calcd.: 773.3; MS Found: 774.2 [M+H]⁺.

### Step F: 4-[(2S)-2-(4-chloro-2-fluorophenyl)-2-methyl-2H-1,3-benzodioxol-4-yl]-1-{[6-(5-ethyl-4H-1,2,4-triazol-3-yl)-3-{[(2S)-oxetan-2-yl]methyl}-3H-imidazo[4,5-b]pyridin-2-yl]methyl}piperidine (Compound 352a)

To a solution of 2-((4-((S)-2-(4-chloro-2-fluorophenyl)-2-methylbenzo[d][1,3]dioxol-4-yl)piperidin-1-yl)methyl)-6-(5-ethyl-4-((2-(trimethylsilyl)ethoxy)methyl)-4H-1,2,4-triazol-3-yl)-3-(((S)-oxetan-2-yl)methyl)-3H-imidazo[4,5-b]pyridine (10 mg, 0.013 mmol) in THE (2 mL) was dropwise added TBAF (1.0 M, 4 mL, THE solution), the mixture was stirred at 60° C. for 4 hrs. The mixture was diluted with EA, washed with H₂O and brine, dried over Na₂SO₄, concentrated, purified by prep-HPLC (0.1% TFA) to give 4-[(2S)-2-(4-chloro-2-fluorophenyl)-2-methyl-2H-1,3-benzodioxol-4-yl]-1-{[6-(5-ethyl-4H-1,2,4-triazol-3-yl)-3-{[(2S)-oxetan-2-yl]methyl}-3H-imidazo[4,5-b]pyridin-2-yl]methyl}piperidine (Compound 352a) (3.5 mg, yield 42%) as a yellow solid.

MS Calcd.: 643.2; MS Found: 644.2 [M+H]⁺.

¹H NMR (400 MHz, CD3OD-d4): δ 9.04 (s, 1H), 8.59 (s, 1H), 7.58 (t, J=8.4 Hz, 1H), 7.30-7.18 (m, 2H), 6.80-6.68 (m, 3H), 5.39-5.30 (m, 2H), 4.73-4.56 (m, 2H), 4.49-4.40 (m, 2H), 4.08-3.98 (m, 2H), 3.10-2.98 (m, 2H), 2.82-2.64 (m, 2H), 2.62-2.50 (m, 1H), 2.40-2.23 (m, 2H), 2.08-1.75 (m, 8H), 0.90 (t, J=7.2 Hz, 3H). ¹⁹F NMR (377 MHz): −112.37.

### Example 250: 3-fluoro-4-[(2S)-2-methyl-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]benzonitrile (Compound 447a)

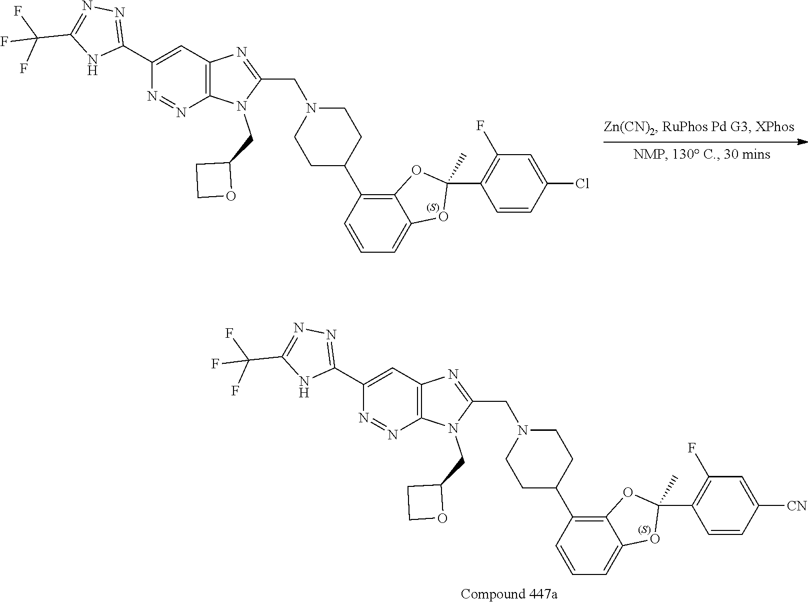

A mixture of 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-3-(5-(trifluoromethyl)-4H-1,2,4-triazol-3-yl)-7H-imidazo[4,5-c]pyridazine (30 mg, 0.044 mmol), Zn(CN)₂ (10.3 mg, 0.088 mmol), RuPhos Pd G₃ (3.7 mg, 0.0044 mmol) and X-Phos (4.2 mg, 0.0088 mmol) in anhydrous NMP (2.0 mL) was stirred at 130° C. for 30 mins under Ar. the reaction mixture was filtered and the filtrate purified by Prep-HPLC (0.1% FA/H₂O/CH₃CN) to give 3-fluoro-4-[(2S)-2-methyl-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]benzonitrile (Compound 447a) (13 mg, yield: 44%) as a white solid.

MS Calcd.: 675.2; MS Found: 676.1 [M+H]⁺.

¹H NMR (400 MHz, DMSO-d6): δ 8.54 (s, 1H), 7.98 (d, J=10.4 Hz, 1H), 7.73-7.80 (m, 2H), 6.73-6.86 (m, 3H), 5.23-5.35 (m, 1H), 5.00-5.09 (m, 1H), 4.86-4.95 (m, 1H), 4.39-4.56 (m, 2H), 4.00-4.18 (m, 2H), 2.90-3.10 (m, 2H), 2.63-2.80 (m, 2H), 2.45-2.53 (m, 1H), 2.25-2.40 (m, 2H), 2.06 (s, 3H), 1.70-1.87 (m, 4H). ¹⁹F NMR (377 MHz): −63.68, −111.06.

### Example 251: 4-[(2S)-2-(4-chloro-2-fluorophenyl)-2-methyl-2H-1,3-benzodioxol-4-yl]-1-{[3-(5-methyl-4H-1,2,4-triazol-3-yl)-7-{[(2S)-oxetan-2-yl]methyl}-7H-imidazo[4,5-c]pyridazin-6-yl]methyl}piperidine (Compound 280a)

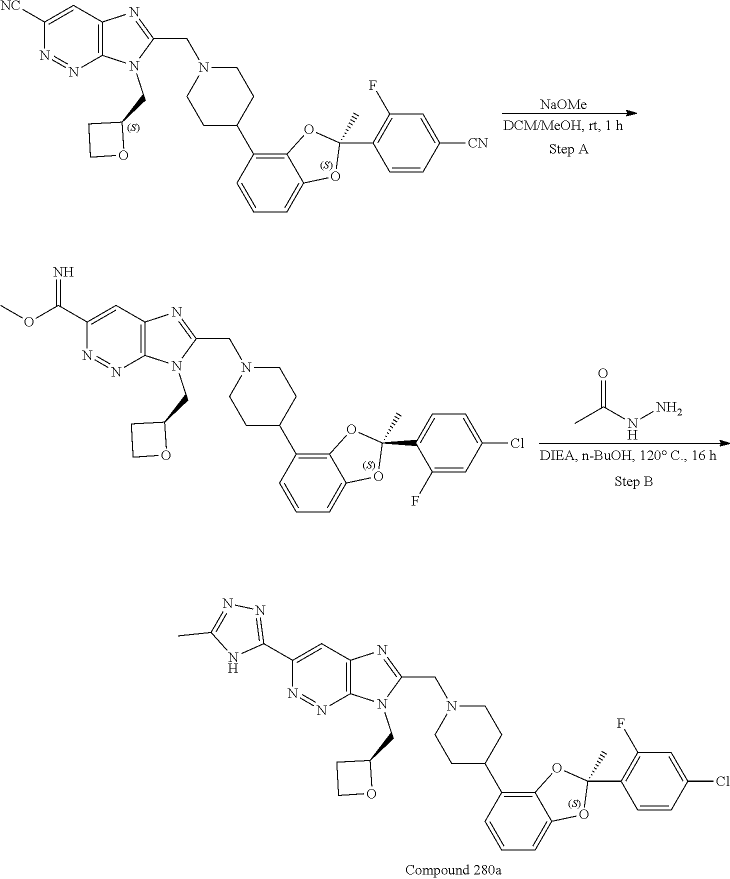

### Step A: methyl 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carbimidate

A mixture of 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carbonitrile (250 mg, 0.44 mmol), sodium methoxide (118 mg, 2.18 mmol) in DCM/MeOH (5.0 mL/5.0 mL) was stirred at room temperature for 1 hour. After the reaction was completed, the mixture was concentrated in vacuum. The residue was purified by column chromatography on silica gel (DCM/MeOH=20/1) to give methyl 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carbimidate (280 mg, crude) as a yellow solid. MS Calcd.: 606.2; MS Found: 607.2 [M+H]⁺.

### Step B: 4-[(2S)-2-(4-chloro-2-fluorophenyl)-2-methyl-2H-1,3-benzodioxol-4-yl]-1-{[3-(5-methyl-4H-1,2,4-triazol-3-yl)-7-{[(2S)-oxetan-2-yl]methyl}-7H-imidazo[4,5-c]pyridazin-6-yl]methyl}piperidine (Compound 280a)

A mixture of methyl 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carbimidate (140 mg, 0.23 mmol), acetohydrazide (51 mg, 0.69 mmol), DIEA (148 mg, 1.15 mmol) in n-BuOH (5.0 mL) was stirred at 120° C. for 16 hours. After the reaction was completed, the reaction mixture was concentrated and the residue purified by Prep-HPLC (0.1% FA/H₂O/CH₃CN) to give 4-[(2S)-2-(4-chloro-2-fluorophenyl)-2-methyl-2H-1,3-benzodioxol-4-yl]-1-{[3-(5-methyl-4H-1,2,4-triazol-3-yl)-7-{[(2S)-oxetan-2-yl]methyl}-7H-imidazo[4,5-c]pyridazin-6-yl]methyl}piperidine (Compound 280a) (10 mg, yield: 7.0%) as a white solid.

MS Calcd.: 630.2; MS Found: 631.2 [M+H]⁺.

¹H NMR (400 MHz, DMSO-d₆): δ 8.37 (s, 1H), 7.52-7.61 (m, 2H), 7.34 (dd, J=8.8 Hz, 1.6 Hz, 1H), 6.72-6.82 (m, 3H), 5.24-5.32 (m, 1H), 4.97-5.05 (m, 1H), 4.83-4.91 (m, 1H), 4.38-4.55 (m, 2H), 4.07 (d, J=14.0 Hz, 1H), 4.00 (d, J=13.2 Hz, 1H), 2.95-3.07 (m, 2H), 2.62-2.80 (m, 2H), 2.50-2.61 (m, 1H), 2.43 (s, 3H), 2.25-2.38 (m, 2H), 2.03 (s, 3H), 1.70-1.87 (m, 4H). ¹⁹F NMR (377 MHz): −110.78.

### Example 252: 5-[6-({4-[(2S)-2-(4-chloro-2-fluorophenyl)-2-methyl-2H-1,3-benzodioxol-4-yl]piperidin-1-yl}methyl)-7-{[(2S)-oxetan-2-yl]methyl}-7H-imidazo[4,5-c]pyridazin-3-yl]-4H-1,2,4-triazole-3-carbonitrile (Compound 448a)

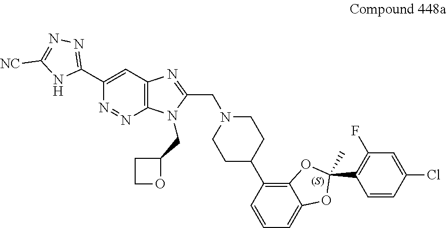

The synthesis of Compound 448a was similar to that of Compound 444a except 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carbonitrile was used instead of 2-((4-((S)-2-(4-chloro-2-fluorophenyl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile. 5-[6-({4-[(2S)-2-(4-chloro-2-fluorophenyl)-2-methyl-2H-1,3-benzodioxol-4-yl]piperidin-1-yl}methyl)-7-{[(2S)-oxetan-2-yl]methyl}-7H-imidazo[4,5-c]pyridazin-3-yl]-4H-1,2,4-triazole-3-carbonitrile (Compound 448a) (7.7 mg) was finally obtained as a white solid.

MS Calcd.: 641.2; MS Found: 642.1 [M+H]⁺.

¹H NMR (400 MHz, DMSO-d₆): δ 8.50 (s, 1H), 7.52-7.61 (m, 2H), 7.34 (dd, J=8.4 Hz, 2.0 Hz, 1H), 6.72-6.82 (m, 3H), 5.25-5.32 (m, 1H), 4.98-5.07 (m, 1H), 4.86-4.94 (m, 1H), 4.39-4.55 (m, 2H), 4.00-4.12 (m, 2H), 2.95-3.10 (m, 2H), 2.50-2.80 (m, 3H), 2.26-2.40 (m, 2H), 2.03 (s, 3H), 1.70-1.90 (m, 4H). ¹⁹F NMR (377 MHz): −110.77.

### Example 253: 5-chloro-2-[(2S)-2-methyl-4-{1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}-2H-1,3-benzodioxol-2-yl]pyridine (Compound 449a)

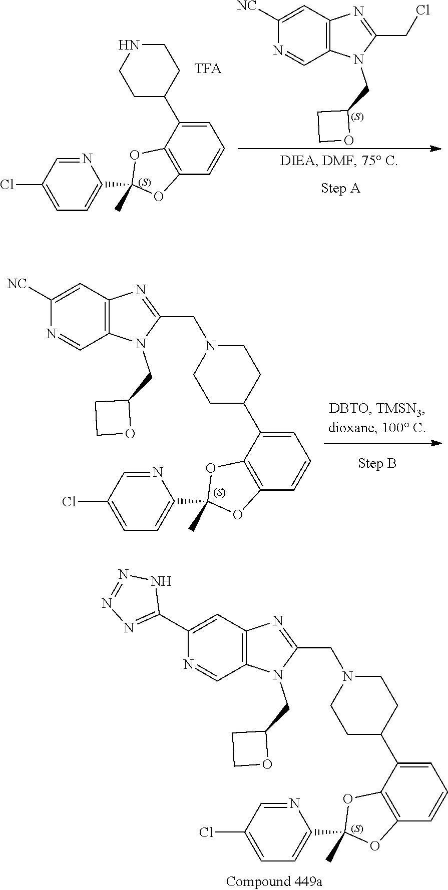

### Step A: 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile

To a solution of (S)-5-chloro-2-(2-methyl-4-(piperidin-4-yl)benzo[d][1,3]dioxol-2-yl)pyridine (146 mg, 0.442 mmol) in DMF (2 mL) was added DIEA (173 mg, 1.33 mmol). After 2 minutes, (S)-2-(chloromethyl)-3-(oxetan-2-ylmethyl)-3H-imidazo[4,5-c]pyridine-6-carbonitrile (79 mg, 0.302 mmol) was added. The resulting mixture was heated to 75° C. for an hour. The reaction was quenched with water (10 mL), extracted with ethyl acetate (20 mL×2). The organic layers were combined, dried over Na₂SO₄, filtered and concentrated under vacuum. The residue was purified by column chromatography on silica gel (PE:EtOAc=1:2) to give 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (150 mg, 61% yield) as a brown solid. MS Calcd.: 556.2; MS Found: 557.6 [M+H]⁺.

### Step B: 5-chloro-2-[(2S)-2-methyl-4-{1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}-2H-1,3-benzodioxol-2-yl]pyridine (Compound 449a)

To a solution of 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (150 mg, 0.270 mmol) in dioxane (2 mL) was added dibutyltin oxide (135 mg, 0.540 mmol) and TMSN₃ (93 mg, 0.810 mmol). The resulting mixture was heated to 100° C. for 4 hours under microwave irradiation under Ar. The solvent was removed in vacuo. The residue was purified by Prep-HPLC to afford 5-chloro-2-[(2S)-2-methyl-4-{1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}-2H-1,3-benzodioxol-2-yl]pyridine (Compound 449a) (14 mg, yield: 9.1%) as a white solid.

MS Calcd.: 599.2; MS Found: 600.1 [M+H]⁺.

¹H NMR (400 MHz, DMSO-d₆): δ 9.09 (br s, 1H), 8.71 (d, J=2.0 Hz, 1H), 8.20-8.26 (m, 1H), 8.00 (dd, J=8.4 Hz, 2.4 Hz, 1H), 7.60 (d, J=8.4, 1H), 6.70-6.82 (m, 3H), 5.13-5.24 (m, 1H), 4.80-4.87 (m, 1H), 4.65-4.73 (m, 1H), 4.45-4.53 (m, 1H), 4.35-4.45 (m, 1H), 3.93-4.00 (m, 1H), 3.80-3.86 (m, 1H), 3.00-3.07 (m, 1H), 2.85-2.91 (m, 1H), 2.65-2.75 (m, 2H), 2.42-2.51 (m, 1H), 2.15-2.30 (m, 2H), 2.01 (s, 3H), 1.68-1.82 (m, 4H).

### Example 254: 5-chloro-2-(2-methyl-4-{1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}-2H-1,3-benzodioxol-2-yl)pyridine (Compound 450a)

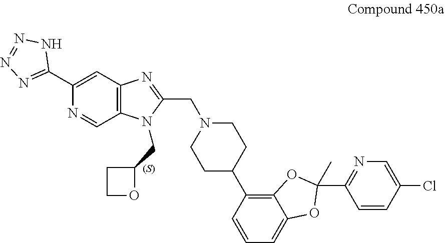

The synthesis of Compound 450a was similar to that of Compound 449a except 5-chloro-2-(2-methyl-4-(piperidin-4-yl)benzo[d][1,3]dioxol-2-yl)pyridine TFA salt was used instead of (S)-5-chloro-2-(2-methyl-4-(piperidin-4-yl)benzo[d][1,3]dioxol-2-yl)pyridine TFA salt. 5-chloro-2-(2-methyl-4-{1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}-2H-1,3-benzodioxol-2-yl)pyridine (Compound 450a) (23.75 mg) was finally obtained as a white solid.

MS Calcd.: 599.2; MS Found: 600.0 [M+H]⁺.

¹H NMR (400 MHz, DMSO-d₆): δ 9.13 (s, 1H), 8.72 (s, 1H), 8.30 (s, 1H), 8.01 (d, J=7.6 Hz, 1H), 7.60 (dd, J=7.4, 2.8 Hz, 1H), 6.72-6.83 (m, 3H), 5.12-5.20 (m, 1H), 4.80-4.90 (m, 1H), 4.67-4.75 (m, 1H), 4.35-4.53 (m, 2H), 3.95-4.03 (m, 1H), 3.80-3.87 (m, 1H), 2.97-3.07 (m, 1H), 2.83-2.92 (m, 1H), 2.56-2.78 (m, 2H), 2.12-2.35 (m, 3H), 2.01 (s, 3H), 1.67-1.82 (m, 4H).

### Example 255: 5-chloro-2-[(2S)-2-methyl-4-{1-[(3-{[(2S)-oxetan-2-yl]methyl}-6-(1H-1,2,3,4-tetrazol-5-yl)-3H-imidazo[4,5-b]pyridin-2-yl)methyl]piperidin-4-yl}-2H-1,3-benzodioxol-2-yl]pyridine (Compound 451a)

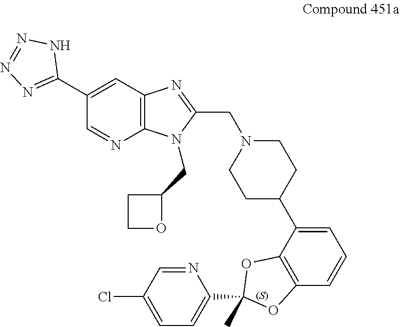

The synthesis of Compound 451a was similar to that of Compound 449a except (S)-2-(chloromethyl)-3-(oxetan-2-ylmethyl)-3H-imidazo[4,5-b]pyridine-6-carbonitrile was used instead of (S)-6-bromo-2-(chloromethyl)-3-(oxetan-2-ylmethyl)-3H-imidazo[4,5-c]pyridine. 5-chloro-2-[(2S)-2-methyl-4-{1-[(3-{[(2S)-oxetan-2-yl]methyl}-6-(1H-1,2,3,4-tetrazol-5-yl)-3H-imidazo[4,5-b]pyridin-2-yl)methyl]piperidin-4-yl}-2H-1,3-benzodioxol-2-yl]pyridine (Compound 451a) (19 mg) was finally obtained as a white solid.

MS Calcd.: 599.2; MS Found: 600.1 [M+H]⁺.

¹H NMR (400 MHz, DMSO-d₆): δ 9.03 (d, J=1.6 Hz, 1H), 8.72 (d, J=2.4 Hz, 1H), 8.59 (d, J=2.0 Hz, 1H), 8.01 (dd, J=8.4 Hz, 2.4 Hz, 1H), 7.62 (d, J=8.8 Hz, 1H), 6.72-6.85 (m, 3H), 5.15-5.23 (m, 1H), 4.77-4.86 (m, 1H), 4.67-4.73 (m, 1H), 4.43-4.51 (m, 1H), 4.32-4.38 (m, 1H), 4.07-4.19 (m, 2H), 3.07-3.20 (m, 2H), 2.63-2.79 (m, 3H), 2.43-2.52 (m, 2H), 2.01 (s, 3H), 1.75-1.90 (m, 4H).

### Example 256: 4-[(2S)-2-(4-chloro-2-fluorophenyl)-2-methyl-2H-1,3-benzodioxol-4-yl]-1-({7-[(2R)-2-methoxypropyl]-3-[5-(trifluoromethyl)-4H-1,2,4-triazol-3-yl]-7H-imidazo[4,5-c]pyridazin-6-yl}methyl)piperidine (Compound 452a)

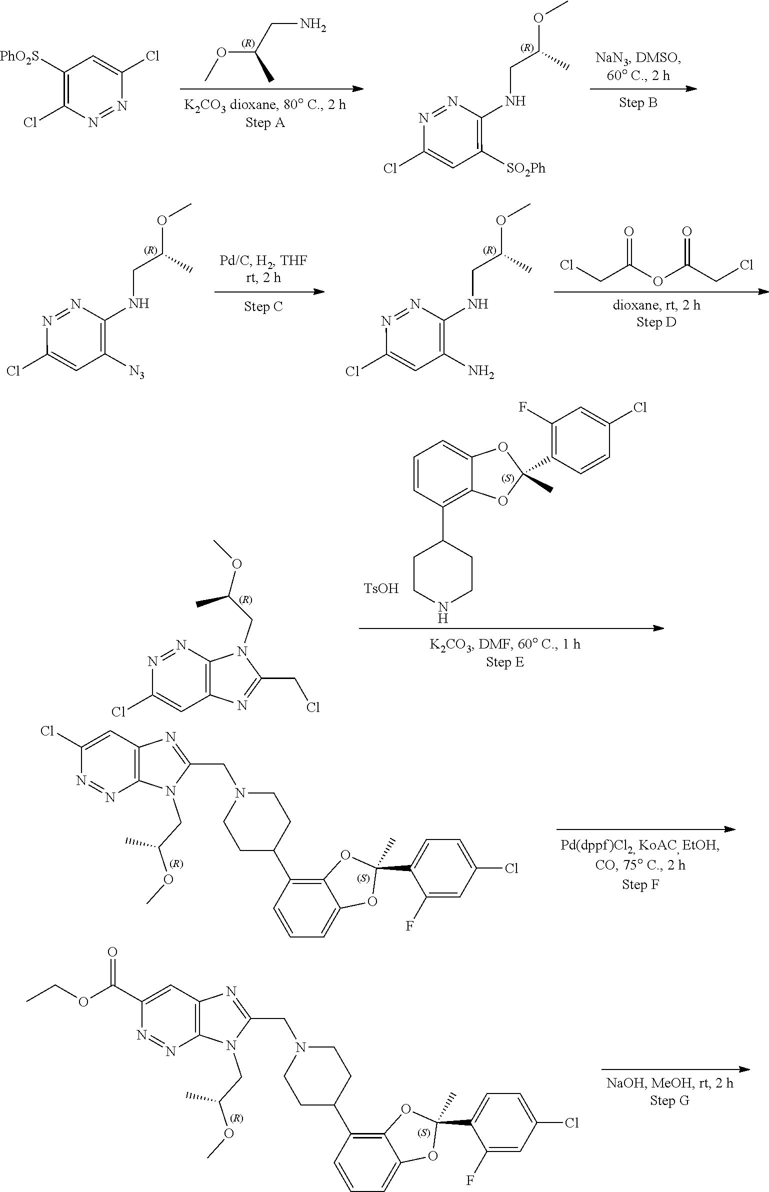

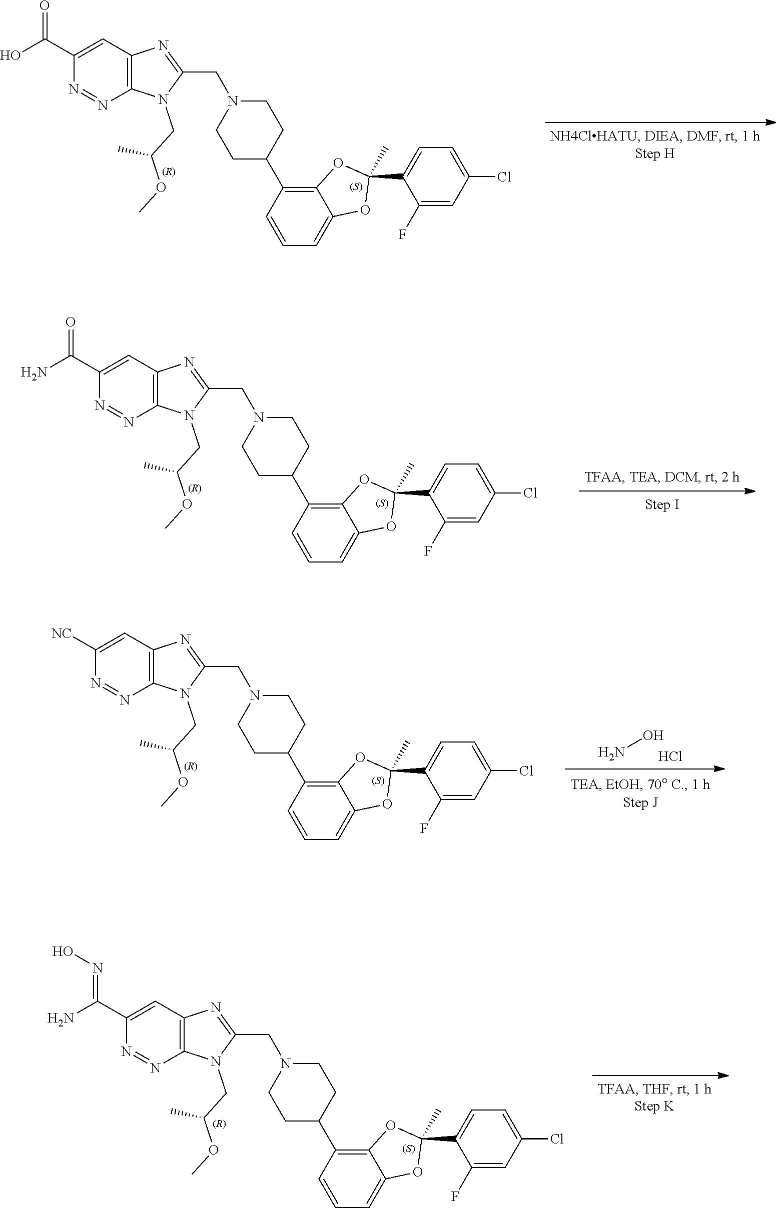

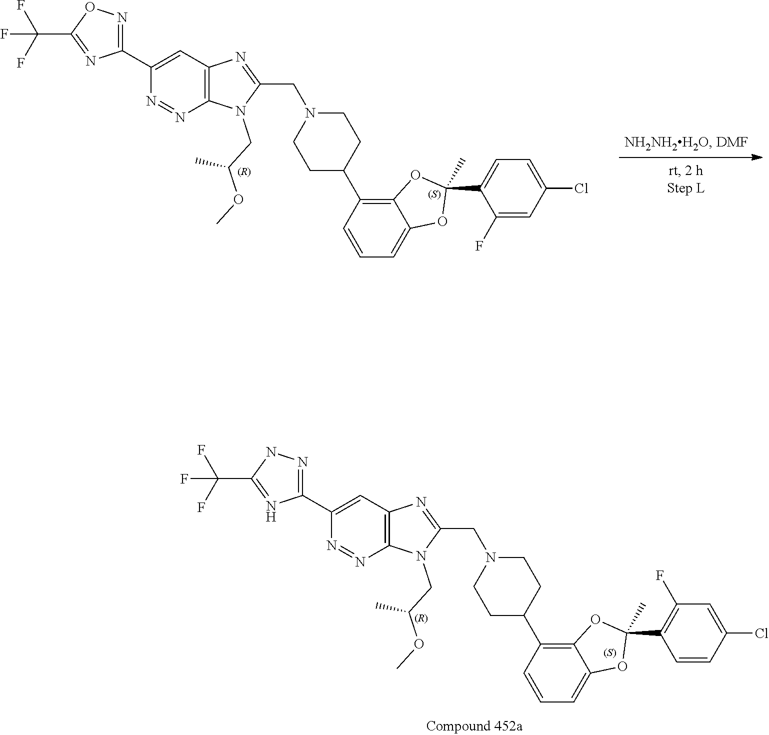

### Step A: (R)-6-chloro-N-(2-methoxypropyl)-4-(phenylsulfonyl)pyridazin-3-amine

A mixture of 3,6-dichloro-4-(phenylsulfonyl)pyridazine (3.03 g, 10.52 mmol), (R)-2-methoxypropan-1-amine (2.14 g, 10.52 mmol), K₂CO₃ (5.79 g, 42.08 mmol) in dioxane (60 mL) was stirred at 80° C. for 2 hours. After the reaction was completed, the reaction mixture was filtered, and the filtrate evaporated to dryness. The residue was purified by column chromatography on silica gel (PE/EA=5/1) to give (R)-6-chloro-N-(2-methoxypropyl)-4-(phenylsulfonyl)pyridazin-3-amine (2.2 g, yield: 33%) as yellow oil. MS Calcd.: 341.1; MS Found: 342.0 [M+H]⁺.

### Step B: (R)-4-azido-6-chloro-N-(2-methoxypropyl)pyridazin-3-amine

To a mixture of (R)-6-chloro-N-(2-methoxypropyl)-4-(phenylsulfonyl)pyridazin-3-amine (2 g, 5.87 mmol) in DMSO (30 mL) was added NaN₃ (1.53 g, 23.46 mmol) and the mixture stirred at 60° C. for 2 hours. After the reaction was completed, the mixture was quenched with H₂O (50 mL) and extracted with EA (100 mL*2). The extracts were wash with brine (100 mL*3). The organic layer was dried over anhydrous sodium sulfate, filtered and evaporated to dryness. The residue was purified by column chromatography on silica gel (PE/EA=3/1) to give (R)-4-azido-6-chloro-N-(2-methoxypropyl)pyridazin-3-amine (1.1 g, yield: 79%) as a yellow solid. MS Calcd.: 242.1; MS Found: 243.1 [M+H]⁺.

### Step C: (R)-6-chloro-N3-(2-methoxypropyl)pyridazine-3,4-diamine

To a solution of (R)-4-azido-6-chloro-N-(2-methoxypropyl)pyridazin-3-amine (1 g, 4.13 mmol) in THE (20 mL) was added Pd/C (300 mg, 10% on Carbon, wetted with ca. 55% water). The mixture was stirred at rt for 2 hours under H₂ (1 atm). The reaction mixture was filtered through a celite pad. The filtrate was evaporated to give (R)-6-chloro-N3-(2-methoxypropyl)pyridazine-3,4-diamine (1.1 g crude) as a brown solid. MS Calcd.: 216.1; MS Found: 217.1 [M+H]⁺.

### Step D: (R)-3-chloro-6-(chloromethyl)-7-(2-methoxypropyl)-7H-imidazo[4,5-c]pyridazine

To a mixture of (R)-6-chloro-N3-(2-methoxypropyl)pyridazine-3,4-diamine (1.0 g, 9.34 mmol) in dioxane (20 mL) was added 2-chloroacetic anhydride (1.58 g, 9.26 mmol). The mixture was stirred at rt for 2 hours. The reaction mixture was evaporated and the residue purified by column chromatography on silica gel (PE/EA=1/1) to give (R)-3-chloro-6-(chloromethyl)-7-(2-methoxypropyl)-7H-imidazo[4,5-c]pyridazine (660 mg, yield: 52%) as yellow oil. MS Calcd.: 274.0; MS Found: 275.0 [M+H]⁺.

### Step E: 3-chloro-6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine

A mixture of (S)-4-(2-(4-chloro-2-fluorophenyl)-2-methylbenzo[d][1,3]dioxol-4-yl)piperidine TsOH salt (751.53 mg, 1.45 mmol), (R)-3-chloro-6-(chloromethyl)-7-(2-methoxypropyl)-7H-imidazo[4,5-c]pyridazine (300 mg, 1.20 mmol), K₂CO₃ (497 mg, 3.60 mmol) in DMF (6 mL) was stirred at 60° C. for 1 hour. After the reaction was completed, the mixture was diluted with EA (40 mL), washed with H₂O (30 mL*2). The combined organic layer was dried over anhydrous sodium sulfate, filtered and evaporated to dryness. The residue was purified by column chromatography on silica gel (DCM/MeOH=80/1) to give 3-chloro-6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine (700 mg, yield: 99%) as yellow oil. MS Calcd.: 585.2; MS Found: 586.2 [M+H]⁺.

### Step F: ethyl 6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine-3-carboxylate

To a mixture of 3-chloro-6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine (760 mg, 1.30 mmol) in EtOH (10 mL) was added PdCl₂(dppf) (190.24 mg, 0.26 mmol) and KOAc (382.2 mg, 3.90 mmol). The mixture was stirred at 75° C. for 2 hours under CO. After the reaction was completed, the reaction mixture was filtered and the filtrate evaporated to dryness. The residue was purified by column chromatography on silica gel (DCM/MeOH=80/1) to give ethyl 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate (750 mg, yield: 93%) as brown oil. MS Calcd.: 623.2; MS Found: 624.2 [M+H]⁺.

### Step G: 6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine-3-carboxylic acid

To a mixture of ethyl 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate (750 mg, 1.20 mmol) in MeOH (6 mL) was added NaOH (2 M, 1.5 mL). The mixture was stirred at rt for 2 h. After the reaction was completed, the mixture was adjusted to pH=7 with 1 N HCl (aq.), diluted with water (20 mL) and extracted with DCM/MeOH (30:1) (30 mL*2). The combined organic layer was dried over anhydrous sodium sulfate, filtered and evaporated to give 6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine-3-carboxylic acid (590 mg, yield: 82%) as a brown solid. MS Calcd.: 595.2; MS Found: 596.2 [M+H]⁺.

### Step H: 6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine-3-carboxamide

A mixture of 6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine-3-carboxylic acid (560 mg), NH₄Cl (74.73 mg, 1.41 mmol), HATU (535.80 mg, 1.41 mmol), DIEA (242.52 mg, 1.88 mmol) in DMF (6 mL) was stirred at rt for 1 hour. After the reaction was completed, the mixture was diluted with EA (30 mL), washed with H₂O (20 mL*2). The combined organic layer was dried over anhydrous sodium sulfate, filtered and evaporated to dryness. The residue was purified by column chromatography on silica gel (DCM/MeOH=60/1) to give 6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine-3-carboxamide (540 mg, yield: 96%) as a brown solid. MS Calcd.: 594.2; MS Found: 595.2 [M+H]⁺.

### Step I: 6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine-3-carbonitrile

A mixture of 6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine-3-carboxamide (600 mg, 1.01 mmol), TEA (408 mg, 4.04 mmol) and TFAA (636 mg, 3.03 mmol) in DCM (6 mL) was stirred at rt for 2 hours under N₂. After the reaction was completed, the reaction was quenched with saturated sodium bicarbonate (20.0 mL) and extracted with ethyl acetate (40 mL×3). The organic layer was dried over sodium sulfate, filtered and concentrated in vacuum. The residue was purified by column chromatography on silica gel (PE/EA=4/1) to give 6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazine-3-carbonitrile (200 mg, yield: 34%) as a yellow solid. MS Calcd.: 576.2; MS Found: 577.2 [M+H]⁺.

### Step J: 6-((4-((S)-2-(4-chloro-2-fluorophenyl)-2-methylbenzo[d][1,3]dioxol-4-yl)piperidin-1-yl)methyl)-N′-hydroxy-7-((R)-2-methoxypropyl)-7H-imidazo[4,5-c]pyridazine-3-carboximidamide

A solution of 6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazine-3-carbonitrile (200 mg, 0.38 mmol), HONH₂·HCl (144.79 mg, 2.08 mmol), TEA (153.38 mg, 1.52 mmol) in EtOH (4 mL) was stirred at 70° C. for 1 h. After the reaction was completed, the mixture was filtered and the solid dried to give 6-((4-((S)-2-(4-chloro-2-fluorophenyl)-2-methylbenzo[d][1,3]dioxol-4-yl)piperidin-1-yl)methyl)-N′-hydroxy-7-((R)-2-methoxypropyl)-7H-imidazo[4,5-c]pyridazine-3-carboximidamide (206 mg, yield: 97%) as a white solid. MS Calcd.: 609.2; MS Found: 610.2 [M+H]⁺.

### Step K: 3-(6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazin-3-yl)-5-(trifluoromethyl)-1,2,4-oxadiazole

To a solution of 6-((4-((S)-2-(4-chloro-2-fluorophenyl)-2-methylbenzo[d][1,3]dioxol-4-yl)piperidin-1-yl)methyl)-N′-hydroxy-7-((R)-2-methoxypropyl)-7H-imidazo[4,5-c]pyridazine-3-carboximidamide (206 mg) in THF (3 mL) was added TFAA (142.07 mg, 0.68 mmol) and the mixture stirred at rt for 1 hour. After the reaction was completed, the reaction was quenched with sodium bicarbonate aqueous solution (5.0 mL), extracted with ethyl acetate (15 mL×3). The organic layers were combined, dried over Na₂SO₄, filtered and concentrated under vacuum to dryness. The residue was purified by Prep-TLC (PE/EA=2/1) to give 3-(6-((4-((S)-2-(4-chloro-2-fluorophenyl)-2-methylbenzo[d][1,3]dioxol-4-yl)piperidin-1-yl)methyl)-7-((R)-2-methoxypropyl)-7H-imidazo[4,5-c]pyridazin-3-yl)-5-(trifluoromethyl)-1,2,4-oxadiazole (220 mg, yield: 95%) as yellow oil. MS Calcd.: 687.2; MS Found: 688.2 [M+H]⁺.

### Step L: 4-[(2S)-2-(4-chloro-2-fluorophenyl)-2-methyl-2H-1,3-benzodioxol-4-yl]-1-({7-[(2R)-2-methoxypropyl]-3-[5-(trifluoromethyl)-4H-1,2,4-triazol-3-yl]-7H-imidazo[4,5-c]pyridazin-6-yl}methyl)piperidine (Compound 452a)

A mixture of 3-(6-((4-((S)-2-(4-chloro-2-fluorophenyl)-2-methylbenzo[d][1,3]dioxol-4-yl)piperidin-1-yl)methyl)-7-(((S)-oxetan-2-yl)methyl)-7H-imidazo[4,5-c]pyridazin-3-yl)-5-(trifluoromethyl)-1,2,4-oxadiazole (220 mg, 0.32 mmol) and N₂H₄·H₂O (32.02 mg, 0.64 mmol) in DMF (2 mL) was stirred at rt for 2 hours. After the reaction was completed, the mixture was purified directly by Prep-HPLC (0.1% TFA/H₂O/CH₃CN) to give 4-[(2S)-2-(4-chloro-2-fluorophenyl)-2-methyl-2H-1,3-benzodioxol-4-yl]-1-({7-[(2R)-2-methoxypropyl]-3-[5-(trifluoromethyl)-4H-1,2,4-triazol-3-yl]-7H-imidazo[4,5-c]pyridazin-6-yl}methyl)piperidine (Compound 452a) (45 mg, yield: 21%) as a yellow solid.

MS Calcd.: 686.2; MS Found: 687.2 [M+H]⁺.

¹H NMR (400 MHz, DMSO-d6): δ 8.62 (s, 1H), 7.53-7.65 (m, 2H), 7.35 (d, J=8.0 Hz, 1H), 6.74-6.89 (m, 3H), 4.80-5.07 (m, 1H), 4.69-4.77 (m, 1H), 4.53-4.64 (m, 1H), 3.71-4.03 (m, 3H), 3.15-3.21 (m, 4H), 2.90-3.05 (m, 1H), 1.90-2.28 (m, 9H), 1.24 (d, J=6.4 Hz, 3H). ¹⁹F NMR (377 MHz): −63.66, −110.50.

### Example 257: 2-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-c]pyridin-6-yl}-6-methyl-3,4-dihydropyrimidin-4-one (Compound 453a)

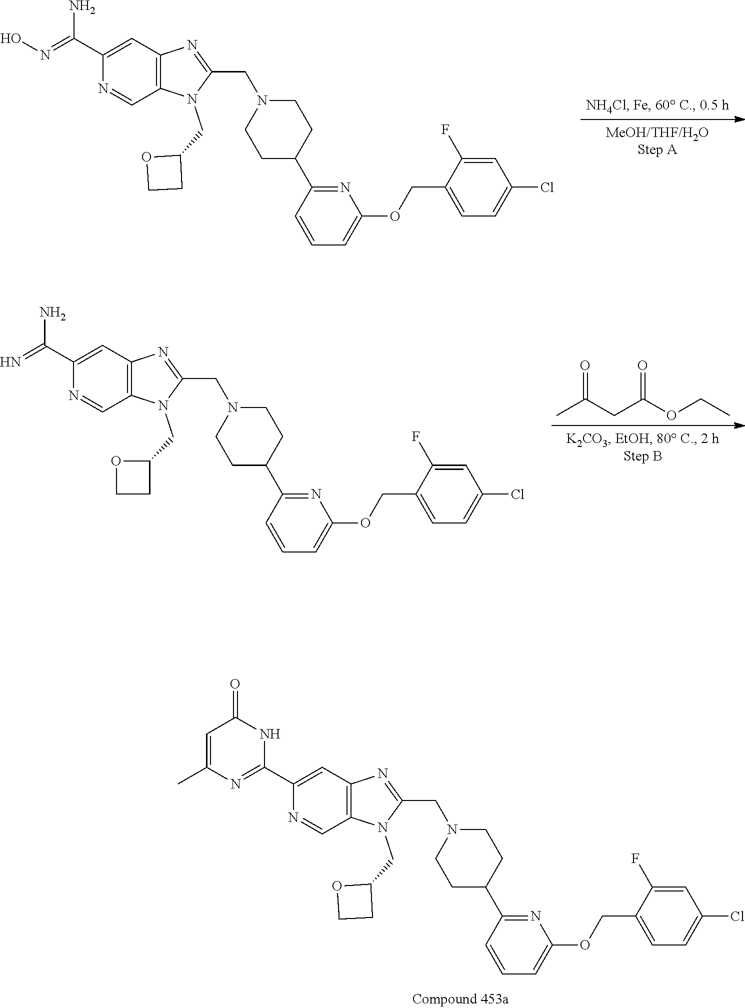

### Step A: (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carboximidamide

To a solution of (S)-2-((4-(6-((4-chloro-2-fluorobenzyl)oxy)pyridin-2-yl)piperidin-1-yl)methyl)-N′-hydroxy-3-(oxetan-2-ylmethyl)-3H-imidazo[4,5-c]pyridine-6-carboximidamide (150 mg, 0.26 mmol) and NH₄Cl (82 mg, 1.55 mmol) in MeOH/THF/H₂O (2 mL/5 mL/0.5 mL) was added Fe (145 mg, 2.59 mmol). The mixture was stirred at 60° C. for 0.5 h. After the reaction was completed, the mixture was filtered, diluted with EA (10 mL), washed with brine (10 mL×3). The organic layer was dried over anhydrous sodium sulfate, filtered and evaporated to dryness. The residue was purified by Prep-TLC (DCM/MeOH=5/1) to give (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carboximidamide (140 mg, 96% yield) as a brown solid. MS Calcd.: 563.2; MS Found: 564.1 [M+H]⁺.

### Step B: 2-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-c]pyridin-6-yl}-6-methyl-3,4-dihydropyrimidin-4-one (Compound 453a)

To a mixture of (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carboximidamide (60 mg, 0.11 mmol) and K₂CO₃ (44 mg, 0.32 mmol) in EtOH (2 mL) was added ethyl 3-oxobutanoate (28 mg, 0.21 mmol). The mixture was stirred at 80° C. for 2 hours. After the reaction was completed, the mixture was diluted with EA (10 mL), washed with brine (10 mL×3). The organic layer was dried over anhydrous sodium sulfate, filtered and evaporated to dryness. The residue was purified by Prep-HPLC to give 2-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-c]pyridin-6-yl}-6-methyl-3,4-dihydropyrimidin-4-one (Compound 453a) (19 mg, yield: 24%).

MS Calcd.: 629.2; MS Found: 630.3 [M+H]⁺.

¹H NMR (400 MHz, DMSO-d₆): δ 9.28 (s, 1H), 8.61 (s, 1H), 7.70 (t, J=8.0 Hz, 1H), 7.61 (t, J=8.2 Hz, 1H), 7.48 (dd, J=10.0, 2.0 Hz, 1H), 7.33 (dd, J=8.4, 2.0 Hz, 1H), 6.94 (d, J=6.8 Hz, 1H), 6.75 (d, J=8.0 Hz, 1H), 6.23 (br.s, 1H), 5.41 (s, 2H), 5.13-5.16 (m, 1H), 4.70-5.05 (m, 4H), 4.38-4.55 (m, 2H), 3.68-3.86 (m, 2H), 2.65-3.01 (m, 3H), 2.32-2.43 (m, 2H), 2.32 (s, 3H), 2.03-2.17 (m, 4H). ¹⁹F NMR (377 MHz): −115.20.

### Example 258: 3-fluoro-4-[(2S)-4-[1-({3-[(2S)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]benzonitrile (Compound 454a)

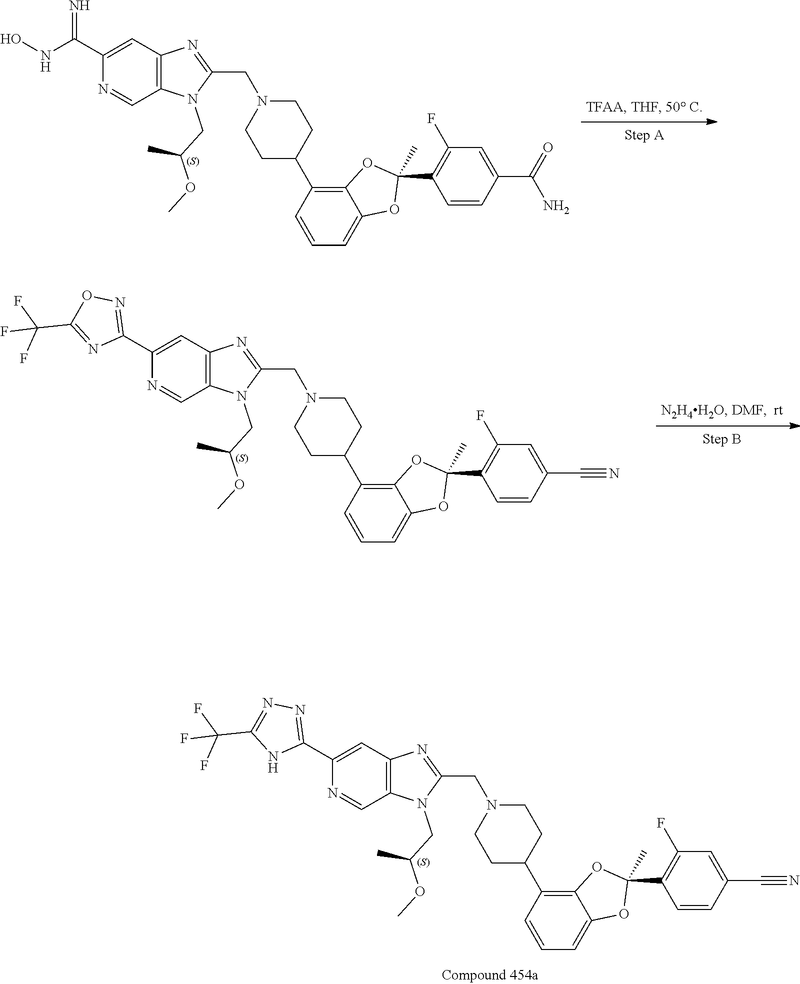

### Step A: 3-fluoro-4-((S)-4-(1-((3-((S)-2-methoxypropyl)-6-(5-(trifluoromethyl)-1,2,4-oxadiazol-3-yl)-3H-imidazo[4,5-c]pyridin-2-yl)methyl)piperidin-4-yl)-2-methylbenzo[d][1,3] dioxol-2-yl)benzonitrile

A solution of 3-fluoro-4-((S)-4-(1-((6-(N-hydroxycarbamimidoyl)-3-((S)-2-methoxypropyl)-3H-imidazo[4,5-c]pyridin-2-yl)methyl)piperidin-4-yl)-2-methylbenzo[d][1,3]dioxol-2-yl)benzamide (160 mg, 0.26 mmol), TFAA (163 mg, 0.78 mmol) in THE (4 mL) was stirred at 50° C. for 7 h. After the reaction was completed, the mixture was diluted with EA (20 mL), washed with Sat. NaHCO₃ (20 mL). The organic layer was dried over anhydrous sodium sulfate, filtered and concentrated. The residue was purified by column chromatography on silica gel (EA/PE=1/20) to give 3-fluoro-4-((S)-4-(1-((3-((S)-2-methoxypropyl)-6-(5-(trifluoromethyl)-1,2,4-oxadiazol-3-yl)-3H-imidazo[4,5-c]pyridin-2-yl)methyl)piperidin-4-yl)-2-methylbenzo[d][1,3]dioxol-2-yl)benzonitrile (50 mg, yield: 28%) as colorless oil. MS Calcd.: 677.2; MS Found: 678.3 [M+H]⁺.

### Step B: 3-fluoro-4-[(2S)-4-[1-({3-[(2S)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]benzonitrile (Compound 454a)

A mixture of 3-fluoro-4-((S)-4-(1-((3-((S)-2-methoxypropyl)-6-(5-(trifluoromethyl)-1,2,4-oxadiazol-3-yl)-3H-imidazo[4,5-c]pyridin-2-yl)methyl)piperidin-4-yl)-2-methylbenzo[d][1,3]dioxol-2-yl)benzonitrile (50 mg, 0.07 mmol), hydrazine hydrate (1 drop) in DMF (1 mL) was stirred at rt for 1 hours. After the reaction was completed, the mixture was filtered and the filtrate was purified by Prep-HPLC to afford 3-fluoro-4-[(2S)-4-[1-({3-[(2S)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]benzonitrile (Compound 454a) (14 mg, yield: 28%) as a white solid.

MS Calcd.: 676.2; MS Found: 677.1 [M+H]⁺.

¹H NMR (400 MHz, DMSO-d₆): δ 8.97-9.50 (m, 1H), 8.27 (s, 1H), 7.96-8.00 (m, 1H), 7.70-7.78 (m, 2H), 6.72-6.85 (m, 3H), 4.30-4.65 (m, 2H), 4.03-4.15 (m, 1H), 3.75-3.92 (m, 2H), 3.05-3.13 (m, 4H), 2.78-2.90 (m, 1H), 2.60-2.71 (m, 1H), 2.15-2.35 (m, 2H), 2.04 (s, 3H), 1.64-1.90 (m, 4H), 1.23 (d, J=4.4 Hz, 3H).

### Example 259: 5-chloro-2-[(2S)-4-[1-({3-[(2R)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]pyridine (Compound 455a)

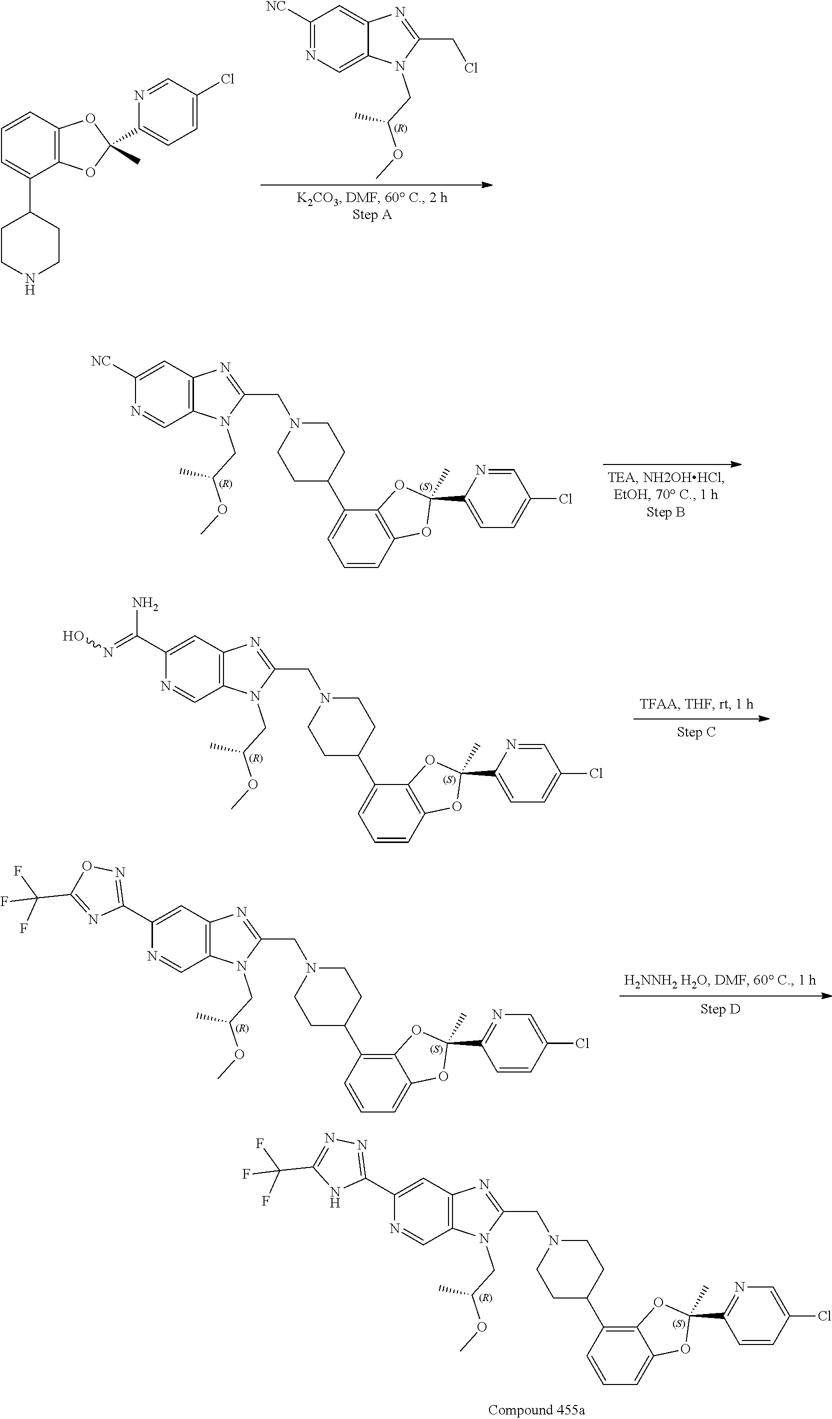

### Step A: 2-((4-((S)-2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridine-6-carbonitrile

A mixture of (S)-5-chloro-2-(2-methyl-4-(piperidin-4-yl)benzo[d][1,3]dioxol-2-yl)pyridine. TFA salt (248 mg, 0.75 mmol), (R)-2-(chloromethyl)-3-(2-methoxypropyl)-3H-imidazo[4,5-c]pyridine-6-carbonitrile (180 mg, 0.68 mmol), K₂CO₃ (375 mg, 2.72 mmol) in DMF (5 mL) was stirred at 60° C. for 2 hours. After the reaction was completed, the mixture was diluted with EA (40 mL), washed with H₂O (30 mL*2). The combined organic layer was dried over anhydrous sodium sulfate, filtered and evaporated to dryness. The residue was purified by column chromatography on silica gel (DCM/MeOH=80/1) to give 2-((4-((S)-2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridine-6-carbonitrile (380 mg, yield: 99%) as yellow oil. MS Calcd.: 558.2; MS Found: 559.2 [M+H]⁺.

### Step B: 2-((4-((S)-2-(5-chloropyridin-2-yl)-2-methylbenzo[d][1,3]dioxol-4-yl)piperidin-1-yl)methyl)-N′-hydroxy-3-((R)-2-methoxypropyl)-3H-imidazo[4,5-c]pyridine-6-carboximidamide

A solution of 2-((4-((S)-2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridine-6-carbonitrile (510 mg, 0.91 mmol), HONH₂·HCl (379 mg, 5.46 mmol), TEA (369.94 mg, 3.66 mmol) in EtOH (10 mL) was stirred at 70° C. for 1 h. After the reaction was completed, the mixture was filtered and the solid dried to give 2-((4-((S)-2-(5-chloropyridin-2-yl)-2-methylbenzo[d][1,3]dioxol-4-yl)piperidin-1-yl)methyl)-N′-hydroxy-3-((R)-2-methoxypropyl)-3H-imidazo[4,5-c]pyridine-6-carboximidamide (360 mg, yield: 67%) as a white solid. MS Calcd.: 591.2; MS Found: 592.2 [M+H]⁺.

### Step C: 3-(2-((4-((S)-2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridin-6-yl)-5-(trifluoromethyl)-1,2,4-oxadiazole

To a solution of 2-((4-((S)-2-(5-chloropyridin-2-yl)-2-methylbenzo[d][1,3]dioxol-4-yl)piperidin-1-yl)methyl)-N′-hydroxy-3-((R)-2-methoxypropyl)-3H-imidazo[4,5-c]pyridine-6-carboximidamide (260 mg, 0.44 mmol) in THE (4 mL) was added TFAA (92.39 mg, 0.88 mmol) and the mixture stirred at rt for 1 hour. After the reaction was completed, the reaction was quenched with sodium bicarbonate aqueous solution (5.0 mL), extracted with ethyl acetate (15 mL×3). The organic layers were combined, dried over Na₂SO₄, filtered and concentrated under vacuum to dryness. The residue was purified by Prep-TLC (PE/EA=1/1) to give 3-(2-((4-((S)-2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridin-6-yl)-5-(trifluoromethyl)-1,2,4-oxadiazole (260 mg, yield: 88%) as yellow oil. MS Calcd.: 669.2; MS Found: 670.2 [M+H]⁺.

### Step D: 5-chloro-2-[(2S)-4-[1-({3-[(2R)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]pyridine (Compound 455a)

A mixture of 3-(2-((4-((S)-2-(5-chloropyridin-2-yl)-2-methylbenzo[d][1,3]dioxol-4-yl)piperidin-1-yl)methyl)-3-((R)-2-methoxypropyl)-3H-imidazo[4,5-c]pyridin-6-yl)-5-(trifluoromethyl)-1,2,4-oxadiazole (260 mg, 0.39 mmol) and N₂H₄·H₂O (38.86 mg, 0.78 mmol) in DMF (2 mL) was stirred at 60° C. for 1 hour. After the reaction was completed, the mixture was purified directly by Prep-HPLC (0.1% FA/H₂O/CH₃CN) to give 5-chloro-2-[(2S)-4-[1-({3-[(2R)-2-methoxypropyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]pyridine (Compound 455a) (118 mg, yield: 46%) as a white solid.

MS Calcd.: 668.2; MS Found: 669.2 [M+H]⁺.

¹H NMR (400 MHz, DMSO-d6): δ 9.07 (s, 1H), 8.70 (d, J=2.4 Hz, 1H), 8.27 (s, 1H), 7.98 (dd, J=8.4 Hz, 2.4 Hz, 1H), 7.58 (d, J=8.0 Hz, 1H), 6.72-6.84 (m, 3H), 4.42-4.57 (m, 2H), 4.06 (d, J=13.6 Hz, 1H), 3.85-3.95 (m, 1H), 3.77 (d, J=13.6 Hz, 1H), 3.02-3.10 (m, 4H), 2.80-2.87 (m, 1H), 2.55-2.70 (m, 1H), 2.23-2.37 (m, 1H), 2.13-2.22 (m, 1H), 2.01 (s, 3H), 1.70-1.87 (m, 4H), 1.20 (d, J=6.4 Hz, 3H). ¹⁹F NMR (377 MHz): −63.70.

### Example 260: 1-{3-[(4-chloro-2-fluorophenyl)methoxy]-4-fluorophenyl}-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 456a)

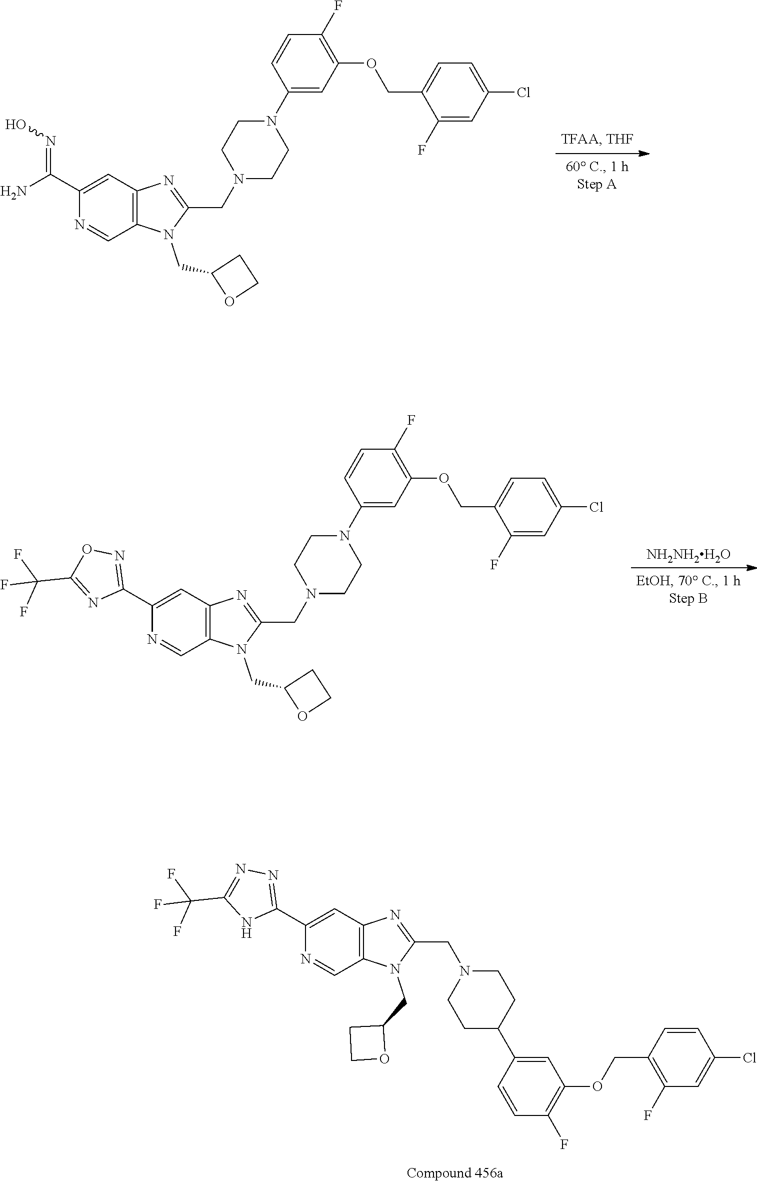 [SMILES: NC(=NO)c1cc2nc(CN3CCN(c4ccc(F)c(OCc5ccc(Cl)cc5F)c4)CC3)n(C[C@@H]3CCO3)c2cn1 ; Fc1cc(Cl)ccc1COc1cc(N2CCN(Cc3nc4cc(-c5noc(C(F)(F)F)n5)ncc4n3C[C@@H]3CCO3)CC2)ccc1F ; "NH2NH2·H2O" ; Fc1cc(Cl)ccc1COc1cc(C2CCN(Cc3nc4cc(-c5nnc(C(F)(F)F)[nH]5)ncc4n3C[C@@H]3CCO3)CC2)ccc1F]

### Step A: (S)-3-(2-((4-(3-((4-chloro-2-fluorobenzyl)oxy)-4-fluorophenyl)piperazin-1-yl)methyl)-3-(oxetan-2-ylmethyl)-3H-imidazo[4,5-c]pyridin-6-yl)-5-(trifluoromethyl)-1,2,4-oxadiazole

A mixture of (S)-2-((4-(3-((4-chloro-2-fluorobenzyl)oxy)-4-fluorophenyl)piperazin-1-yl)methyl)-N′-hydroxy-3-(oxetan-2-ylmethyl)-3H-imidazo[4,5-c]pyridine-6-carboximidamide (50 mg) and TFAA (88 mg, 0.419 mmol) in THE (5 mL) was stirred at 60° C. for 1 h. The mixture was poured into sodium bicarbonate aqueous solution (30 mL) and extracted with EtOAc (2×30 mL), the combined organic layer was washed with brine, dried over sodium sulfate, filtered and the residue was concentrated to obtain (S)-3-(2-((4-(3-((4-chloro-2-fluorobenzyl)oxy)-4-fluorophenyl)piperazin-1-yl)methyl)-3-(oxetan-2-ylmethyl)-3H-imidazo[4,5-c]pyridin-6-yl)-5-(trifluoromethyl)-1,2,4-oxadiazole (56 mg) as a yellow oil. MS Calcd: 675.2; MS Found: 676.2 [M+H]⁺.

### Step B: 1-{3-[(4-chloro-2-fluorophenyl)methoxy]-4-fluorophenyl}-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 456a)

A mixture of (S)-3-(2-((4-(3-((4-chloro-2-fluorobenzyl)oxy)-4-fluorophenyl)piperazin-1-yl)methyl)-3-(oxetan-2-ylmethyl)-3H-imidazo[4,5-c]pyridin-6-yl)-5-(trifluoromethyl)-1,2,4-oxadiazole (56 mg) and NH₂NH2·H₂O (0.2 mL) in EtOH (2 mL) was stirred at 70° C. for 1 h. The mixture was purified by prep-HPLC to obtain 1-{3-[(4-chloro-2-fluorophenyl)methoxy]-4-fluorophenyl}-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 456a) (10 mg, yield: 18%) as a white solid.

MS Calcd: 674.2; MS Found: 675.2 [M+H]⁺.

¹H NMR (400 MHz, DMSO-d₆) 9.18 (s, 1H), 8.30 (s, 1H), 7.59 (t, J=8.0 Hz, 1H), 7.50 (dd, J=1.6 Hz, 10.0 Hz, 1H), 7.36 (dd, J=1.2 Hz, 8.4 Hz, 1H), 7.05 (dd, J=2.4 Hz, 11.2 Hz, 1H), 6.85 (dd, J=2.4 Hz, 7.6 Hz, 1H), 6.50-6.44 (m, 1H), 5.21 (s, 2H), 5.20-5.13 (m, 1H), 4.97-4.88 (m, 1H), 4.80-4.73 (m, 1H), 4.55-4.49 (m, 1H), 4.48-4.40 (m, 1H), 4.10-3.88 (m, 2H), 3.11 (t, J=1.2 Hz, 4H), 2.80-2.70 (m, 1H), 2.69-2.58 (m, 4H), 2.52-2.40 (m, 1H). ¹⁹F NMR (377 MHz): −63.62, −115.00, −146.06.

### Example 261: 4-[3-(4-chloro-2-fluorophenyl)-2,3-dihydro-1,4-benzodioxin-5-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 457a)

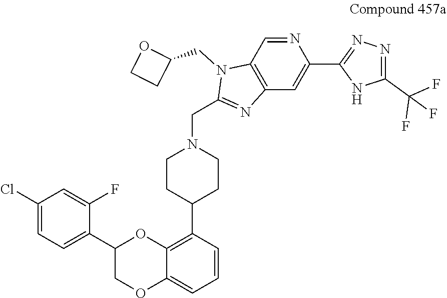 [SMILES: Fc1cc(Cl)ccc1C1COc2cccc(C3CCN(Cc4nc5cc(-c6nnc(C(F)(F)F)[nH]6)ncc5n4C[C@@H]4CCO4)CC3)c2O1]

The synthesis of Compound 457a was similar to that of Compound 361a except 4-(3-(4-chloro-2-fluorophenyl)-2,3-dihydrobenzo[b][1,4]dioxin-5-yl)piperidine TFA salt was used instead of 4-(2-(4-chlorophenyl)-2-methylbenzo[d][1,3]dioxol-4-yl)piperidine. 4-[3-(4-chloro-2-fluorophenyl)-2,3-dihydro-1,4-benzodioxin-5-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 457a) (3.4 mg) was finally obtained as a white solid.

MS Calcd.: 683.2; MS Found: 684.3 [M+H]⁺.

¹H NMR (400 MHz, DMSO-d₆): δ 9.14 (s, 1H), 8.27 (s, 1H), 7.50-7.60 (m, 2H), 7.40-7.45 (m, 1H), 6.77-6.87 (m, 3H), 5.45-5.50 (m, 1H), 5.10-5.20 (m, 1H), 4.86-4.95 (m, 1H), 4.71-4.78 (m, 1H), 4.37-4.57 (m, 3H), 4.08-4.17 (m, 1H), 3.93-4.01 (m, 1H), 3.80-3.90 (m, 1H), 2.96-3.04 (m, 1H), 2.80-2.93 (m, 1H), 2.60-2.80 (m, 2H), 2.38-2.53 (m, 1H), 2.11-2.32 (m, 2H), 1.50-1.83 (m, 4H). ¹⁹F NMR (377 MHz): −63.48, −63.49, −115.06, −115.08.

### Example 262: 4-[2-(4-chloro-2-fluorophenyl)-5-fluoro-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 458a)

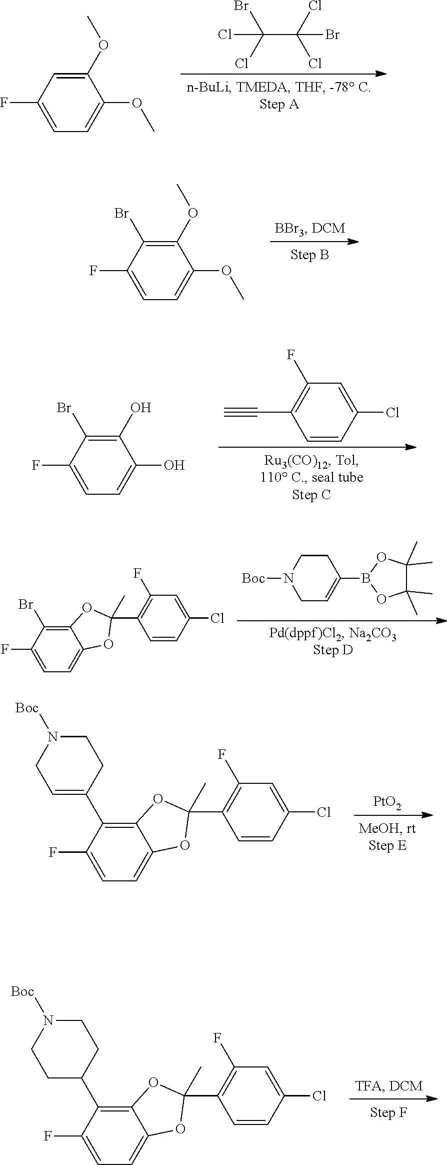

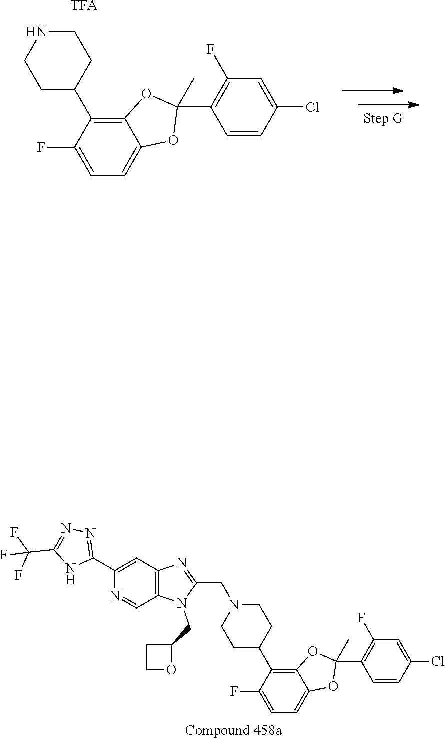 [SMILES: CC1(c2ccc(Cl)cc2F)Oc2ccc(F)c(C3CCNCC3)c2O1 ; "TFA" ; CC1(c2ccc(Cl)cc2F)Oc2ccc(F)c(C3CCN(Cc4nc5cc(-c6nnc(C(F)(F)F)[nH]6)ncc5n4C[C@@H]4CCO4)CC3)c2O1]

### Step A: 2-bromo-1-fluoro-3,4-dimethoxybenzene

To a solution of 4-fluoro-1,2-dimethoxybenzene (2.0 g, 12.8 mmol) and TMEDA (1.5 g, 12.8 mmol) in THF (20 mL) was added n-BuLi (12 mL, 1.6 mol/L, 19.2 mmol) at −78° C. The reaction was stirred at −78° C. for 1.5 hours. Then 1,2-dibromo-1,1,2,2-tetrachloroethane (5.4 g, 16.7 mmol) was added and the reaction was stirred at −78° C. for 2 h. After the reaction was completed, the reaction was quenched with NH₄Cl solution (50 mL) and extracted with ethyl acetate (100 mL×3). The organic layer was combined and washed with brine (100 mL×2), dried over sodium sulfate, filtered and concentrated in vacuum. The crude product was purified by column chromatography on silica gel (PE:EA=10:1) to give 2-bromo-1-fluoro-3,4-dimethoxybenzene (2.5 g, crude) as colorless oil.

### Step B: 3-bromo-4-fluorobenzene-1,2-diol

To a solution of 2-bromo-1-fluoro-3,4-dimethoxybenzene (2.5 g, 10.7 mmol) in DCM (25 mL) was added BBr₃ (5.34 g, 21.4 mmol) at room temperature. The reaction was stirred at room temperature for 2 hours. After the reaction was completed, the reaction was quenched with MeOH (100 mL) and concentrated in vacuum. The residue was purified by column chromatography (PE:EA=3:1) to give 3-bromo-4-fluorobenzene-1,2-diol (1.4 g, 53% yield in 2 steps) as a dark brown solid.

### Step C: 4-bromo-2-(4-chloro-2-fluorophenyl)-5-fluoro-2-methylbenzo[d][1,3]dioxole

A mixture of 3-bromo-4-fluorobenzene-1,2-diol (1.4 g, 6.8 mmol) and 4-chloro-1-ethynyl-2-fluorobenzene (1.25 g, 8.2 mmol) and Ru₃(CO)₁₂ (217 mg, 0.34 mmol) in toluene (15 mL) was degassed and charged with Ar. The reaction was stirred at 110° C. for 16 hours in sealed tube. After the reaction was completed, the reaction was concentrated in vacuum. The residue was purified by column chromatography (PE:EA=10:1) to give 4-bromo-2-(4-chloro-2-fluorophenyl)-5-fluoro-2-methylbenzo[d][1,3]dioxole (500 mg, 21% yield) as yellow oil.

### Step D: tert-butyl 4-(2-(4-chloro-2-fluorophenyl)-5-fluoro-2-methylbenzo[d][1,3]dioxol-4-yl)-3,6-dihydropyridine-1(2H)-carboxylate

A mixture of 4-bromo-2-(4-chloro-2-fluorophenyl)-5-fluoro-2-methylbenzo[d][1,3]dioxole (300 mg, 0.8 mmol) and tert-butyl 4-(4,4,5,5-tetramethyl-1,3,2-dioxaborolan-2-yl)-3,6-dihydropyridine-1(2H)-carboxylate (309 mg, 0.96 mmol), Pd(dppf)Cl₂·CH₂Cl₂ (68 mg, 0.08 mmol) and Na₂CO₃ (265 mg, 2.4 mmol) in dioxane/H₂O (6 mL/0.6 mL) was degassed and charged with N₂. The reaction was stirred at 90° C. for 16 hours. After the reaction was completed, the reaction was quenched with water (50 mL) and extracted with ethyl acetate (100 mL×3). The organic layer was combined and washed with brine (50 mL×2), dried over sodium sulfate, filtered and concentrated in vacuum. The residue was purified by column chromatography on silica gel (PE:EA=10:1) to give tert-butyl 4-(2-(4-chloro-2-fluorophenyl)-5-fluoro-2-methylbenzo[d][1,3]dioxol-4-yl)-3,6-dihydropyridine-1(2H)-carboxylate (180 mg, 47% yield) as a yellow solid. MS Calcd.: 463.1; MS Found: 486.1 [M+Na]⁺.

### Step E: tert-butyl 4-(2-(4-chloro-2-fluorophenyl)-5-fluoro-2-methylbenzo[d][1,3]dioxol-4-yl)piperidine-1-carboxylate

To a solution of tert-butyl 4-(2-(4-chloro-2-fluorophenyl)-5-fluoro-2-methylbenzo[d][1,3]dioxol-4-yl)-3,6-dihydropyridine-1(2H)-carboxylate (180 mg, 0.39 mmol) in MeOH (2 mL) was added PtO₂ (50 mg, 0.22 mmol). The reaction was stirred at room temperature under hydrogen atmosphere for 0.5 hour. After the reaction was completed, the reaction was filtered and concentrated in vacuum to give tert-butyl 4-(2-(4-chloro-2-fluorophenyl)-5-fluoro-2-methylbenzo[d][1,3]dioxol-4-yl)piperidine-1-carboxylate (180 mg, crude) as yellow oil. MS Calcd.: 465.2; MS Found: 488.3 [M+Na]⁺.

### Step F: 4-(2-(4-chloro-2-fluorophenyl)-5-fluoro-2-methylbenzo[d][1,3]dioxol-4-yl)piperidine TFA salt

To a solution of tert-butyl 4-(2-(4-chloro-2-fluorophenyl)-5-fluoro-2-methylbenzo[d][1,3]dioxol-4-yl)piperidine-1-carboxylate (180 mg) in DCM (3 mL) was added TFA (1 mL) at room temperature. The reaction was stirred at room temperature for 1 hour. After the reaction was completed, the reaction was concentrated in vacuum to give 4-(2-(4-chloro-2-fluorophenyl)-5-fluoro-2-methylbenzo[d][1,3]dioxol-4-yl)piperidine TFA salt (176 mg, crude) as yellow oil.

### Step G: 4-[2-(4-chloro-2-fluorophenyl)-5-fluoro-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 458a)

The synthesis of Compound 458a was similar to that of Compound 361a except 4-(2-(4-chloro-2-fluorophenyl)-5-fluoro-2-methylbenzo[d][1,3]dioxol-4-yl)piperidine TFA salt was used instead of 4-(2-(4-chlorophenyl)-2-methylbenzo[d][1,3]dioxol-4-yl)piperidine. 4-[2-(4-chloro-2-fluorophenyl)-5-fluoro-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 458a) (35 mg) was finally obtained as a white solid.

MS Calcd.: 701.2; MS Found: 702.1 [M+H]⁺.

¹H NMR (400 MHz, DMSO-d₆): δ 9.17 (s, 1H), 8.29 (s, 1H), 7.52-7.60 (m, 2H), 7.35 (dd, J=8.8 Hz, J=2.0 Hz, 1H), 6.80 (dd, J=8.4 Hz, J=4.0 Hz, 1H), 6.64 (dd, J=10.8 Hz, J=8.8 Hz, 1H), 5.15-5.25 (m, 1H), 4.88-4.97 (m, 1H), 4.70-4.80 (m, 1H), 4.40-4.53 (m, 2H), 3.97-4.06 (m, 1H), 3.80-3.89 (m, 1H), 3.26-3.31 (m, 1H), 3.00-3.10 (m, 1H), 2.78-2.93 (m, 2H), 2.66-2.77 (m, 1H), 2.12-2.35 (m, 2H), 2.04 (s, 3H), 1.90-2.10 (m, 2H), 1.58-1.73 (m, 2H). ¹⁹F NMR (377 MHz): −63.73, −110.86, −110.93, −126.49, −126.53.

### Example 263: 4-[2-(4-chloro-2-fluorophenyl)-7-fluoro-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 459a)

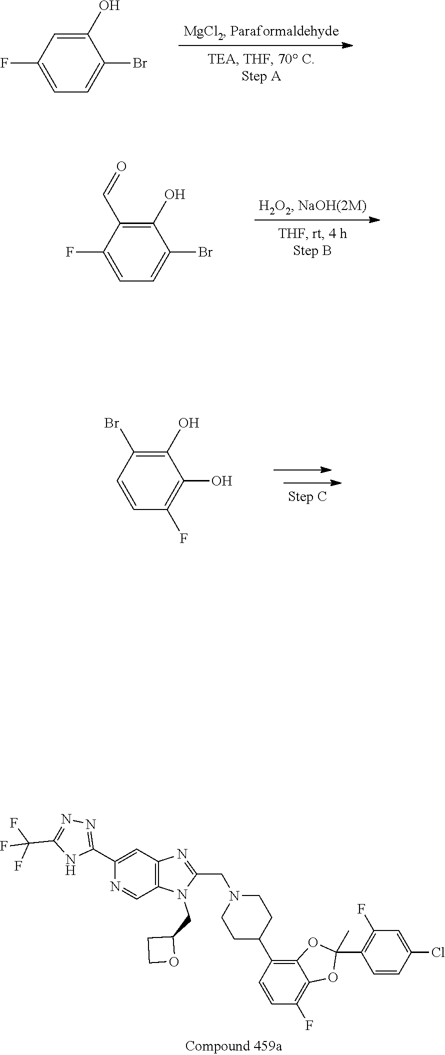 [SMILES: Oc1cc(F)ccc1Br ; O=Cc1c(F)ccc(Br)c1O ; Oc1c(F)ccc(Br)c1O ; CC1(c2ccc(Cl)cc2F)Oc2c(F)ccc(C3CCN(Cc4nc5cc(-c6nnc(C(F)(F)F)[nH]6)ncc5n4C[C@@H]4CCO4)CC3)c2O1]

### Step A: 3-bromo-6-fluoro-2-hydroxybenzaldehyde

To a solution of MgCl₂ (7.9 g, 83.8 mmol) and paraformaldehyde (3.8 g, 125.6 mmol) in THE (100 mL) was added TEA (12.7 g, 125.6 mmol) at room temperature. The reaction was stirred at room temperature for 1 hour. 2-bromo-5-fluorophenol (8.0 g, 41.9 mmol) was added and the reaction was stirred at 70° C. for 4 hours. After the reaction was completed, the reaction was quenched with HCl (1M, 30 mL) and extracted with ethyl acetate (100 mL×3). The organic layer was combined and washed with brine (50 mL×2), dried over sodium sulfate, filtered and concentrated in vacuum. The residue was purified by column chromatography (PE:EA=10:1) to give 3-bromo-6-fluoro-2-hydroxybenzaldehyde (4.8 g, 49% yield) as a yellow oil.

¹H NMR (400 MHz, DMSO-d₆): δ 10.74 (s, 1H), 7.50 (dd, J=8.8, 6.4 Hz, 1H), 6.75 (dd, J=10.5, 2.9 Hz, 1H), 6.62 (td, J=8.6, 2.9 Hz, 1H).

### Step B: 3-bromo-6-fluorobenzene-1,2-diol

To a solution of 3-bromo-6-fluoro-2-hydroxybenzaldehyde (4.8 g, 22.0 mmol) in THE (50 mL) was added H₂O₂ (30% aqueous, 2 mL) and NaOH (2 M, 2 mL) at room temperature. The reaction was stirred at room temperature for 4 hours. After the reaction was completed, the reaction was quenched with saturated NaHSO₃ (30 mL) and extracted with ethyl acetate (50 mL×3). The organic layer was combined and washed with brine (30 mL×2), dried over sodium sulfate, filtered and concentrated in vacuum. The residue was purified by column chromatography on silica gel (PE) to give 3-bromo-6-fluorobenzene-1,2-diol (2.0 g, 44% yield) as a green solid.

¹H NMR (400 MHz, DMSO-d₆): δ 9.81 (s, 1H), 9.65 (s, 1H), 6.94 (dd, J=9.0, 5.6 Hz, 1H), 6.64 (dd, J=10.2, 9.0 Hz, 1H).

### Step C: 4-[2-(4-chloro-2-fluorophenyl)-7-fluoro-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 459a)

The synthesis of Compound 459a was similar to that of Compound 458a except 3-bromo-6-fluorobenzene-1,2-diol was used instead of 3-bromo-4-fluorobenzene-1,2-diol. 4-[2-(4-chloro-2-fluorophenyl)-7-fluoro-2-methyl-2H-1,3-benzodioxol-4-yl]-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 459a) (6.4 mg) was finally obtained as a white solid.

MS Calcd.: 701.2; MS Found: 702.1 [M+H]⁺.

¹H NMR (400 MHz, DMSO-d₆): δ 9.15 (s, 1H), 8.27 (s, 1H), 7.55-7.62 (m, 2H), 7.37 (d, J=8.4 Hz, 1H), 6.72-6.82 (m, 2H), 5.15-5.25 (m, 1H), 4.86-4.95 (m, 1H), 4.70-4.78 (m, 1H), 4.38-4.55 (m, 2H), 4.00 (d, J=14.0 Hz, 1H), 3.85 (d, J=13.6 Hz, 1H), 2.99-3.07 (m, 1H), 2.84-2.90 (m, 1H), 2.68-2.80 (m, 1H), 2.57-2.68 (m, 1H), 2.40-2.53 (m, 1H), 2.13-2.35 (m, 2H), 2.08 (s, 3H), 1.67-1.80 (m, 4H). ¹⁹F NMR (377 MHz): −63.46, −110.70, −110.73, −141.40, −141.41.

### Example 264: 3-{2-[(4-{2-[(4-chloro-2-fluorophenyl)methoxy]pyrimidin-4-yl}piperidin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 460a)

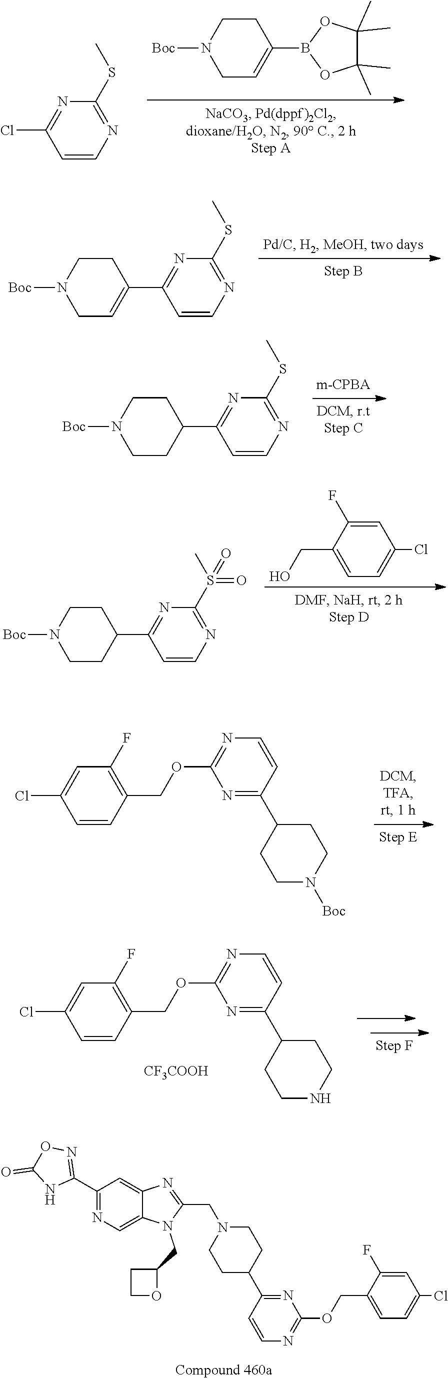

### Step A: tert-butyl 4-(2-(methylthio)pyrimidin-4-yl)-3,6-dihydropyridine-1(2H)-carboxylate

To a solution of 4-chloro-2-(methylthio)pyrimidine (1.1 g, 6.85 mmol), tert-butyl 4-(4,4,5,5-tetramethyl-1,3,2-dioxaborolan-2-yl)-3,6-dihydropyridine-1(2H)-carboxylate (2.5 g, 8.22 mmol) and Na₂CO₃ (2.2 g, 20.55 mmol) in dioxane/H₂O (30 mL/3 mL) was added Pd(dppf)₂Cl₂ (251 mg, 0.34 mmol). The mixture was stirred at 90° C. for 2 hours under N₂. After the reaction was completed, the mixture was filtered, diluted with EA (50 mL), washed with brine (50 mL×3). The organic layer was dried over anhydrous sodium sulfate, filtered and evaporated to dryness. The residue was purified by column chromatography on silica gel (PE/EA=5/1) to give tert-butyl 4-(2-(methylthio)pyrimidin-4-yl)-3,6-dihydropyridine-1(2H)-carboxylate (2.0 g, 95% yield) as a colorless oil. MS Calcd.: 307.1; MS Found: 308.0 [M+H]⁺.

### Step B: tert-butyl 4-(2-(methylthio)pyrimidin-4-yl)piperidine-1-carboxylate

A mixture of tert-butyl 4-(2-(methylthio)pyrimidin-4-yl)-3,6-dihydropyridine-1(2H)-carboxylate (750 mg, 2.44 mmol) and Pd/C (1.5 g, 10% on carbon, wetted with ca. 55% water) in MeOH (10 mL) was stirred at room temperature under H₂ (1 atm) for 2 days. After the reaction was completed, the mixture was filtered and the filtrate evaporated to dryness to give tert-butyl 4-(2-(methylthio)pyrimidin-4-yl)piperidine-1-carboxylate (430 mg, 57% yield) as colorless oil. MS Calcd.: 309.2; MS Found: 310.5 [M+H]⁺.

### Step C: tert-butyl 4-(2-(methylsulfonyl)pyrimidin-4-yl)piperidine-1-carboxylate

To a solution of tert-butyl 4-(2-(methylthio)pyrimidin-4-yl)piperidine-1-carboxylate (430 mg) in DCM (4 mL) was added m-CPBA (718 mg, 4.16 mmol). The mixture was stirred at room temperature for 16 hours. After the reaction was completed, the mixture was diluted with DCM (30 mL), washed with sat Na₂CO₃ (30 mL×1) and water (30 mL×2). The organic layer was dried over anhydrous sodium sulfate, filtered and evaporated to dryness. The residue was purified by column chromatography on silica gel (PE/EA=5/1) to give tert-butyl 4-(2-(methylsulfonyl)pyrimidin-4-yl)piperidine-1-carboxylate (335 mg, 71% yield) as a white solid. MS Calcd.: 341.1; MS Found: 364.1 [M+23]⁺.

### Step D: tert-butyl 4-(2-((4-chloro-2-fluorobenzyl)oxy)pyrimidin-4-yl)piperidine-1-carboxylate

To a solution of (4-chloro-2-fluorophenyl)methanol (141 mg, 0.88 mmol) in DMF (4 mL) was added NaH (53 mg, 60% purity, 1.32 mmol) at 0° C. tert-butyl 4-(2-(methylsulfonyl)pyrimidin-4-yl)piperidine-1-carboxylate (300 mg, 0.88 mmol) was added 0.5 h later. The mixture was stirred at room temperature for 2 hours. After the reaction was completed, the mixture was diluted with EA (20 mL), washed with brine (20 mL×3). The organic layer was dried over anhydrous sodium sulfate, filtered and evaporated to dryness. The residue was purified by Prep-TLC (PE/EA=2/1) to give tert-butyl 4-(2-((4-chloro-2-fluorobenzyl)oxy)pyrimidin-4-yl)piperidine-1-carboxylate (200 mg, 54% yield) as a colorless oil. MS Calcd.: 421.2; MS Found: 422.1 [M+H]⁺.

### Step E: 2-((4-chloro-2-fluorobenzyl)oxy)-4-(piperidin-4-yl)pyrimidine

To a solution of tert-butyl 4-(2-((4-chloro-2-fluorobenzyl)oxy)pyrimidin-4-yl)piperidine-1-carboxylate (100 mg, 0.24 mmol) in DCM (3 mL) was added TFA (1 mL). The mixture was stirred at room temperature for 1 hour. After the reaction was completed, the mixture was concentrated to give 2-((4-chloro-2-fluorobenzyl)oxy)-4-(piperidin-4-yl)pyrimidine TFA salt (120 mg, crude, ca. 100% yield) as yellow oil. MS Calcd.: 321.1; MS Found: 322.2 [M+H]⁺.

### Step F: 3-{2-[(4-{2-[(4-chloro-2-fluorophenyl)methoxy]pyrimidin-4-yl}piperidin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 460a)

The synthesis of Compound 460a was similar to that of Compound 347a except 2-((4-chloro-2-fluorobenzyl) oxy)-4-(piperidin-4-yl)pyrimidine TFA salt was used instead of 4-(2-(4-chlorophenyl)-2-methylbenzo [d][1,3]dioxol-4-yl)piperidine. 3-{2-[(4-{2-[(4-chloro-2-fluorophenyl)methoxy]pyrimidin-4-yl}piperidin-1-yl)methyl]-3-{[(2S)-oxetan-2-yl]methyl}-3H-imidazo[4,5-c]pyridin-6-yl}-4,5-dihydro-1,2,4-oxadiazol-5-one (Compound 460a) (14.2 mg) was finally obtained as a white solid.

MS Calcd.: 606.2; MS Found: 607.2 [M+H]⁺.

¹H NMR (400 MHz, DMSO-d₆): δ 9.16 (s, 1H), 8.51 (d, J=4.8 Hz, 1H), 8.17 (s, 1H), 7.58 (t, J=8.2 Hz, 1H), 7.49 (dd, J=10.0, 2.0 Hz, 1H), 7.32 (dd, J=8.0, 2.0 Hz, 1H), 7.09 (d, J=5.2 Hz, 1H), 5.40 (s, 2H), 5.10-5.18 (m, 1H), 4.88-4.95 (m, 1H), 4.72-4.78 (m, 1H), 4.37-4.55 (m, 2H), 4.00 (d, J=14.0 Hz, 1H), 3.87 (d, J=14.0 Hz, 1H), 2.98-3.03 (m, 1H), 2.84-2.90 (m, 1H), 2.60-2.80 (m, 2H), 2.40-2.52 (m, 1H), 2.18-2.35 (m, 2H), 1.60-1.88 (m, 4H). ¹⁹F NMR (377 MHz): −115.1.

### Example 265: 5-chloro-2-[(2S)-2-methyl-4-{1-[(7-{[(2S)-oxetan-2-yl]methyl}-3-(1H-1,2,3,4-tetrazol-5-yl)-7H-imidazo[4,5-c]pyridazin-6-yl)methyl]piperidin-4-yl}-2H-1,3-benzodioxol-2-yl]pyridine (Compound 461a)

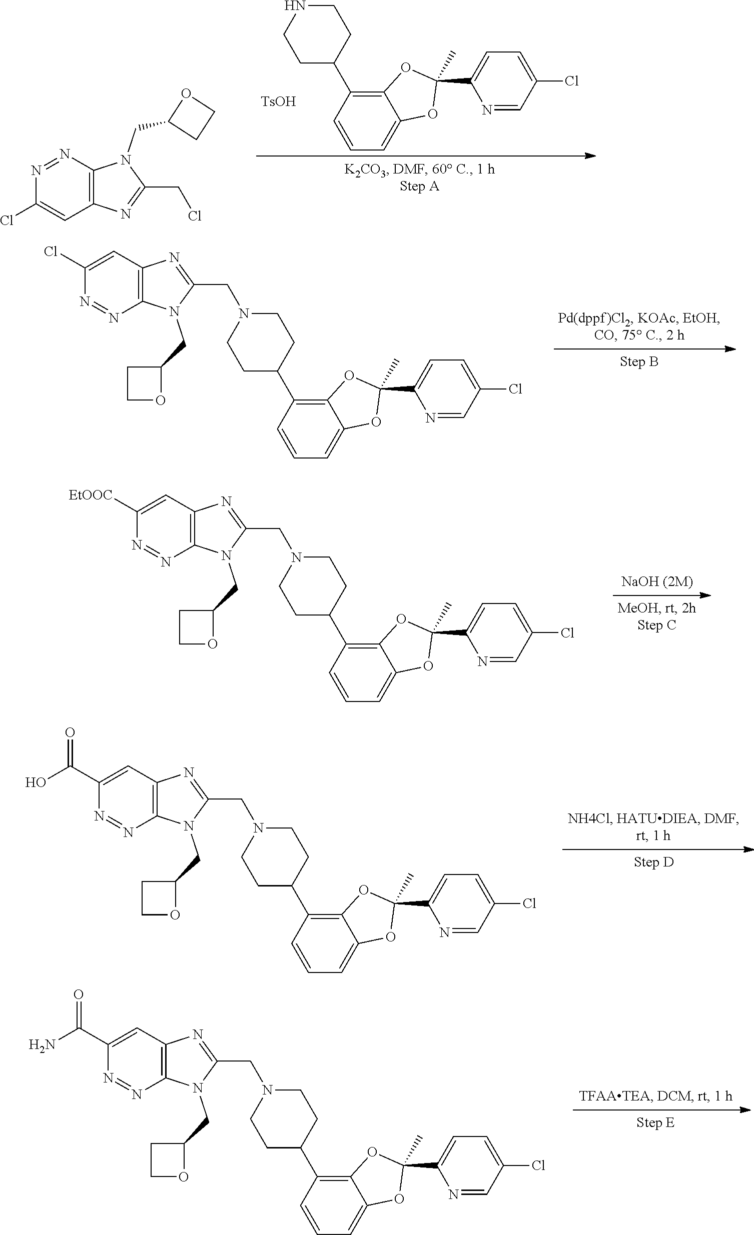

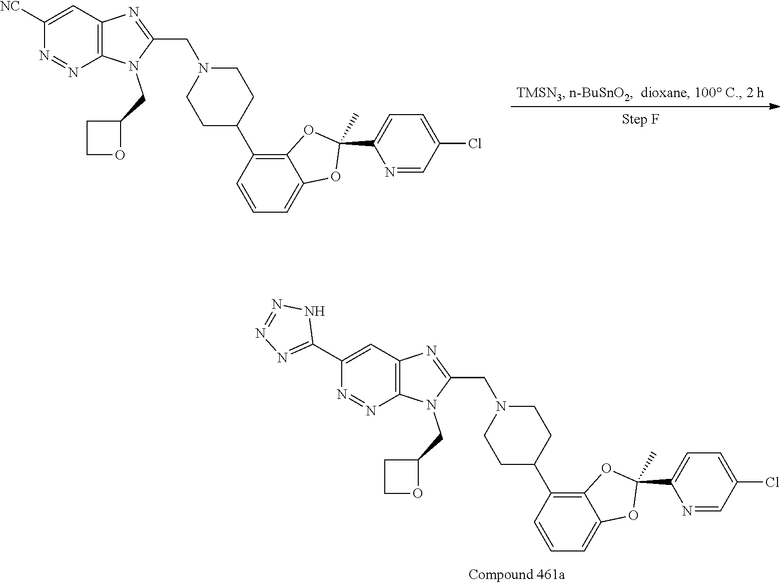 [SMILES: C[C@]1(c2ccc(Cl)cn2)Oc2cccc(C3CCN(Cc4nc5cc(C#N)nnc5n4C[C@@H]4CCO4)CC3)c2O1 ; C[C@]1(c2ccc(Cl)cn2)Oc2cccc(C3CCN(Cc4nc5cc(-c6nnn[nH]6)nnc5n4C[C@@H]4CCO4)CC3)c2O1]

### Step A: 3-chloro-6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine

A mixture of (S)-5-chloro-2-(2-methyl-4-(piperidin-4-yl)benzo[d][1,3]dioxol-2-yl)pyridine. TsOH salt (1.5 g, 4.5 mmol), (S)-3-chloro-6-(chloromethyl)-7-(oxetan-2-ylmethyl)-7H-imidazo[4,5-c]pyridazine (1.3 g, 4.8 mmol), K₂CO₃ (2.6 g, 19.1 mmol) in DMF (15 mL) was stirred at 60° C. for 1 hour. After the reaction was completed, the mixture was diluted with EA (50 mL), washed with H₂O (30 mL*2). The combined organic layer was dried over anhydrous sodium sulfate, filtered and evaporated to dryness. The residue was purified by column chromatography on silica gel (DCM/MeOH=80/1) to give 3-chloro-6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine (700 mg, yield: 99%) as yellow oil. MS Calcd.: 566.2; MS Found: 566.9 [M+H]⁺.

### Step B: ethyl 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate

To a mixture of 3-chloro-6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine (2.3 g, 4.1 mmol) in EtOH (20 mL) was added PdCl₂(dppf) (595 mg, 0.81 mmol) and KOAc (1.2 g, 12.2 mmol). The mixture was stirred at 75° C. for 2 hours under CO. After the reaction was completed, the reaction mixture was filtered and the filtrate evaporated to dryness. The residue was purified by column chromatography on silica gel (DCM/MeOH=80/1) to give ethyl 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate (2.1 g, yield: 87%) as brown oil. MS Calcd.: 604.2; MS Found: 605.2 [M+H]⁺.

### Step C: 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylic acid

To a mixture of ethyl 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate (2.14 g, 3.54 mmol) in MeOH (20 mL) was added NaOH (2 M, 4.0 mL). The mixture was stirred at rt for 2 h. After the reaction was completed, the mixture was adjusted to pH=7 with 1 N HCl (aq.), diluted with water (20 mL) and extracted with DCM/MeOH (30:1) (30 mL*2). The combined organic layer was dried over anhydrous sodium sulfate, filtered and evaporated to give 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylic acid (2.1 g, crude) as a brown solid. MS Calcd.: 576.2; MS Found: 577.2 [M+H]⁺.

### Step D: 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxamide

A mixture of 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylic acid (2.0 g, 3.47 mmol), NH₄Cl (276 mg, 5.21 mmol), HATU (1.97 g, 5.21 mmol), DIEA (895.26 mg, 6.94 mmol) in DMF (20 mL) was stirred at rt for 1 hour. After the reaction was completed, the mixture was diluted with EA (80 mL), washed with H₂O (40 mL*2). The combined organic layer was dried over anhydrous sodium sulfate, filtered and evaporated to dryness. The residue was purified by column chromatography on silica gel (DCM/MeOH=60/1) to give 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxamide (1.9 g, yield: 95%) as a brown solid. MS Calcd.: 575.2; MS Found: 576.2 [M+H]⁺.

### Step E: 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxamide

A mixture of 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxamide (1.2 g, 2.09 mmol), TEA (844 mg, 8.36 mmol) and TFAA (1.3 g, 6.26 mmol) in DCM (10 mL) was stirred at rt for 1 hour under N₂. After the reaction was completed, the reaction was quenched with saturated sodium bicarbonate (25.0 mL) and extracted with ethyl acetate (50 mL×3). The organic layer was dried over sodium sulfate, filtered and concentrated in vacuum. The residue was purified by column chromatography on silica gel (DCM/MeOH=60/1) to give 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carbonitrile (560 mg, yield: 48%) as a yellow solid. MS Calcd.: 557.2; MS Found: 558.2 [M+H]⁺.

### Step F: 5-chloro-2-[(2S)-2-methyl-4-{1-[(7-{[(2S)-oxetan-2-yl]methyl}-3-(1H-1,2,3,4-tetrazol-5-yl)-7H-imidazo[4,5-c]pyridazin-6-yl)methyl]piperidin-4-yl}-2H-1,3-benzodioxol-2-yl]pyridine (Compound 461a)

A mixture of 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carbonitrile (60 mg, 0.11 mmol), TMSN₃ (61.94 mg, 0.54 mmol) and n-BuSnO₂ (54.77 mg, 0.22 mmol) in dioxane (1.5 mL) was heated at 100° C. for 2 hours under an atmosphere of nitrogen. The solvent was removed in vacuo. The crude was purified by Prep-HPLC (0.1% FA/H₂O/CH₃CN) to give 5-chloro-2-[(2S)-2-methyl-4-{1-[(7-{[(2S)-oxetan-2-yl]methyl}-3-(1H-1,2,3,4-tetrazol-5-yl)-7H-imidazo[4,5-c]pyridazin-6-yl)methyl]piperidin-4-yl}-2H-1,3-benzodioxol-2-yl]pyridine (Compound 461a) (19 mg, yield: 29%) as a purple solid.

MS Calcd.: 600.2; MS Found: 601.1 [M+H]⁺.

¹H NMR (400 MHz, DMSO-d6): δ 8.72 (d, J=2.4 Hz, 1H), 8.36 (s, 1H), 8.01 (dd, J=8.4 Hz, 2.4 Hz, 1H), 7.62 (d, J=8.4 Hz, 1H), 6.75-6.84 (m, 3H), 5.24-5.33 (m, 1H), 4.93-5.01 (m, 1H), 4.80-4.88 (m, 1H), 4.45-4.53 (m, 1H), 4.35-4.43 (m, 1H), 3.97-4.07 (m, 2H), 2.95-3.07 (m, 2H), 2.62-2.78 (m, 2H), 2.50-2.62 (m, 1H), 2.25-2.35 (m, 2H), 2.02 (s, 3H), 1.72-1.88 (m, 4H).

### Example 266: 5-chloro-2-[(2S)-2-methyl-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]pyridine (Compound 462a)

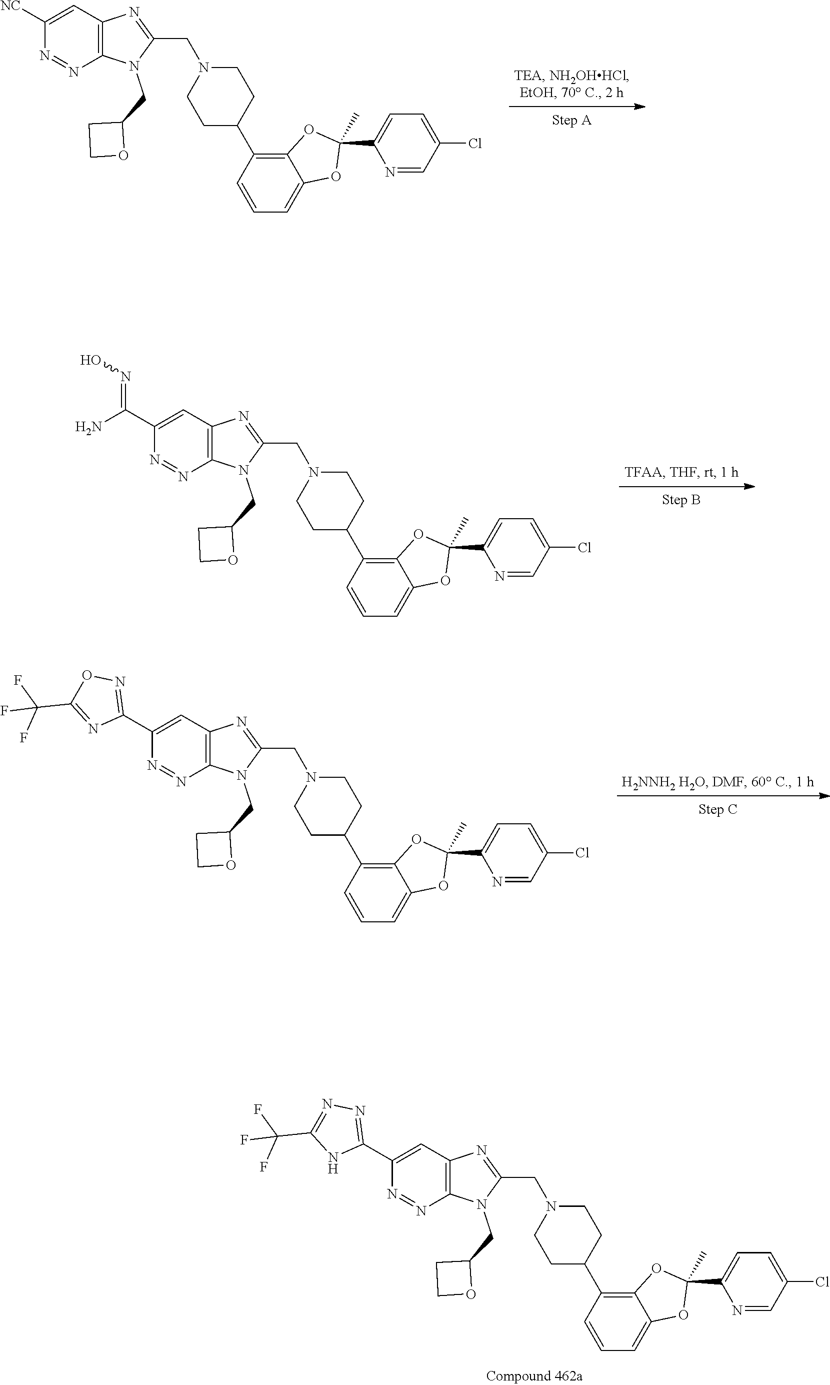 [SMILES: C[C@]1(c2ccc(Cl)cn2)Oc2cccc(C3CCN(Cc4nc5cc(C#N)nnc5n4C[C@@H]4CCO4)CC3)c2O1 ; C[C@]1(c2ccc(Cl)cn2)Oc2cccc(C3CCN(Cc4nc5cc(C(N)=NO)nnc5n4C[C@@H]4CCO4)CC3)c2O1 ; C[C@]1(c2ccc(Cl)cn2)Oc2cccc(C3CCN(Cc4nc5cc(-c6noc(C(F)(F)F)n6)nnc5n4C[C@@H]4CCO4)CC3)c2O1 ; C[C@]1(c2ccc(Cl)cn2)Oc2cccc(C3CCN(Cc4nc5cc(-c6nnc(C(F)(F)F)[nH]6)nnc5n4C[C@@H]4CCO4)CC3)c2O1]

### Step A: 6-((4-((S)-2-(5-chloropyridin-2-yl)-2-methylbenzo[d][1,3]dioxol-4-yl)piperidin-1-yl)methyl)-N′-hydroxy-7-(((S)-oxetan-2-yl)methyl)-7H-imidazo[4,5-c]pyridazine-3-carboximidamide

A solution of 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carbonitrile (200 mg, 0.36 mmol), HONH₂·HCl (150 mg, 2.16 mmol), TEA (145 mg, 1.44 mmol) in EtOH (4 mL) was stirred at 70° C. for 2 h. After the reaction was completed, the mixture was filtered and the solid dried to give 6-((4-((S)-2-(5-chloropyridin-2-yl)-2-methylbenzo[d][1,3]dioxol-4-yl)piperidin-1-yl)methyl)-N′-hydroxy-7-(((S)-oxetan-2-yl)methyl)-7H-imidazo[4,5-c]pyridazine-3-carboximidamide (210 mg, yield: 99%) as a white solid. MS Calcd.: 590.2; MS Found: 591.2 [M+H]⁺.

### Step B: 3-(6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazin-3-yl)-5-(trifluoromethyl)-1,2,4-oxadiazole

To a solution of 6-((4-((S)-2-(5-chloropyridin-2-yl)-2-methylbenzo[d][1,3]dioxol-4-yl)piperidin-1-yl)methyl)-N′-hydroxy-7-(((S)-oxetan-2-yl)methyl)-7H-imidazo[4,5-c]pyridazine-3-carboximidamide (150 mg,) in THE (3 mL) was added TFAA (107 mg, 0.51 mmol) and the mixture stirred at rt for 1 hour. After the reaction was completed, the reaction was quenched with sodium bicarbonate aqueous solution (5.0 mL), extracted with ethyl acetate (15 mL×3). The organic layers were combined, dried over Na₂SO₄, filtered and concentrated under vacuum to dryness. The residue was purified by Prep-TLC (PE/EA=1/1) to give 3-(6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazin-3-yl)-5-(trifluoromethyl)-1,2,4-oxadiazole (130 mg, yield: 77%) as a yellow oil. MS Calcd.: 668.2; MS Found: 669.2 [M+H]⁺.

### Step C: 5-chloro-2-[(2S)-2-methyl-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]pyridine (Compound 462a)

A mixture of 3-(6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazin-3-yl)-5-(trifluoromethyl)-1,2,4-oxadiazole (130 mg, 0.19 mmol) and N₂H₄·H₂O (19.46 mg, 0.39 mmol) in DMF (2 mL) was stirred at 60° C. for 1 hour. After the reaction was completed, the mixture was purified directly by Prep-HPLC (0.1% FA/H₂O/CH₃CN) to give 5-chloro-2-[(2S)-2-methyl-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]pyridine (Compound 462a) (52 mg, yield: 40%) as a brown solid.

MS Calcd.: 667.2; MS Found: 668.1 [M+H]⁺.

¹H NMR (400 MHz, DMSO-d6): δ 8.72 (d, J=2.4 Hz, 1H), 8.51 (s, 1H), 8.01 (dd, J=8.4 Hz, 2.4 Hz, 1H), 7.61 (d, J=8.4 Hz, 1H), 6.75-6.83 (m, 3H), 5.25-5.33 (m, 1H), 4.97-5.06 (m, 1H), 4.85-4.93 (m, 1H), 4.45-4.53 (m, 1H), 4.38-4.45 (m, 1H), 4.00-4.10 (m, 2H), 2.95-3.07 (m, 2H), 2.62-2.80 (m, 2H), 2.50-2.61 (m, 1H), 2.25-2.37 (m, 2H), 2.01 (s, 3H), 1.70-1.86 (m, 4H). ¹⁹F NMR (377 MHz): −63.54.

### Example 267: 3-[6-({4-[(2S)-2-(5-chloropyridin-2-yl)-2-methyl-2H-1,3-benzodioxol-4-yl]piperidin-1-yl}methyl)-7-{[(2S)-oxetan-2-yl]methyl}-7H-imidazo[4,5-c]pyridazin-3-yl]-4,5-dihydro-1,2,4-oxadiazol-5-one (Compound 463a)

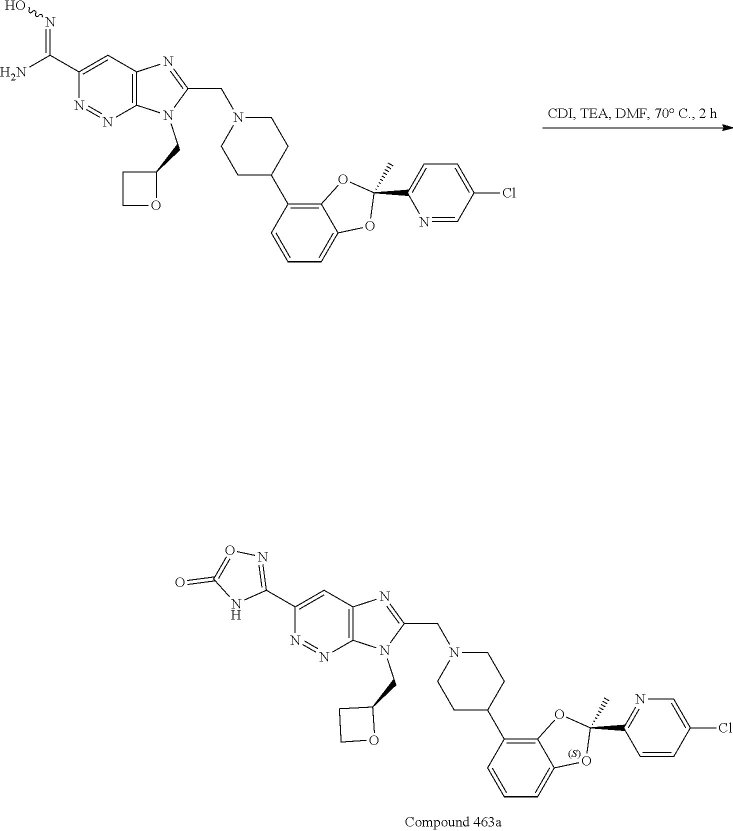

To a solution of 6-((4-((S)-2-(5-chloropyridin-2-yl)-2-methylbenzo[d][1,3]dioxol-4-yl)piperidin-1-yl)methyl)-N′-hydroxy-7-(((S)-oxetan-2-yl)methyl)-7H-imidazo[4,5-c]pyridazine-3-carboximidamide (60 mg, 0.10 mmol) in DMF (1.0 mL) was added TEA (31 mg, 0.31 mmol) and CDI (25 mg, 0.15 mmol). The resulting mixture was stirred at 70° C. for 2 h. After the reaction was completed, the mixture was purified directly by Prep-HPLC (0.1% FA/H₂O/CH₃CN) to give 3-[6-({4-[(2S)-2-(5-chloropyridin-2-yl)-2-methyl-2H-1,3-benzodioxol-4-yl]piperidin-1-yl}methyl)-7-{[(2S)-oxetan-2-yl]methyl}-7H-imidazo[4,5-c]pyridazin-3-yl]-4,5-dihydro-1,2,4-oxadiazol-5-one (Compound 463a) (30 mg, yield: 48%) as a white solid.

MS Calcd.: 616.2; MS Found: 617.1 [M+H]⁺.

¹H NMR (400 MHz, DMSO-d₆): δ 8.72 (d, J=1.6 Hz, 1H), 8.42 (s, 1H), 8.01 (dd, J=8.4 Hz, 2.4 Hz, 1H), 7.61 (d, J=8.4 Hz, 1H), 6.73-6.82 (m, 3H), 5.24-5.31 (m, 1H), 4.95-5.04 (m, 1H), 4.84-4.90 (m, 1H), 4.43-4.52 (m, 1H), 4.35-4.42 (m, 1H), 4.00-4.10 (m, 2H), 2.95-3.06 (m, 2H), 2.60-2.78 (m, 2H), 2.50-2.57 (m, 1H), 2.25-2.37 (m, 2H), 2.01 (s, 3H), 1.70-1.85 (m, 4H).

### Example 268: 6-({4-[(2S)-2-(5-chloropyridin-2-yl)-2-methyl-2H-1,3-benzodioxol-4-yl]piperidin-1-yl}methyl)-7-{[(2S)-oxetan-2-yl]methyl}-7H-imidazo[4,5-c]pyridazine-3-carboxylic acid (Compound 464a)

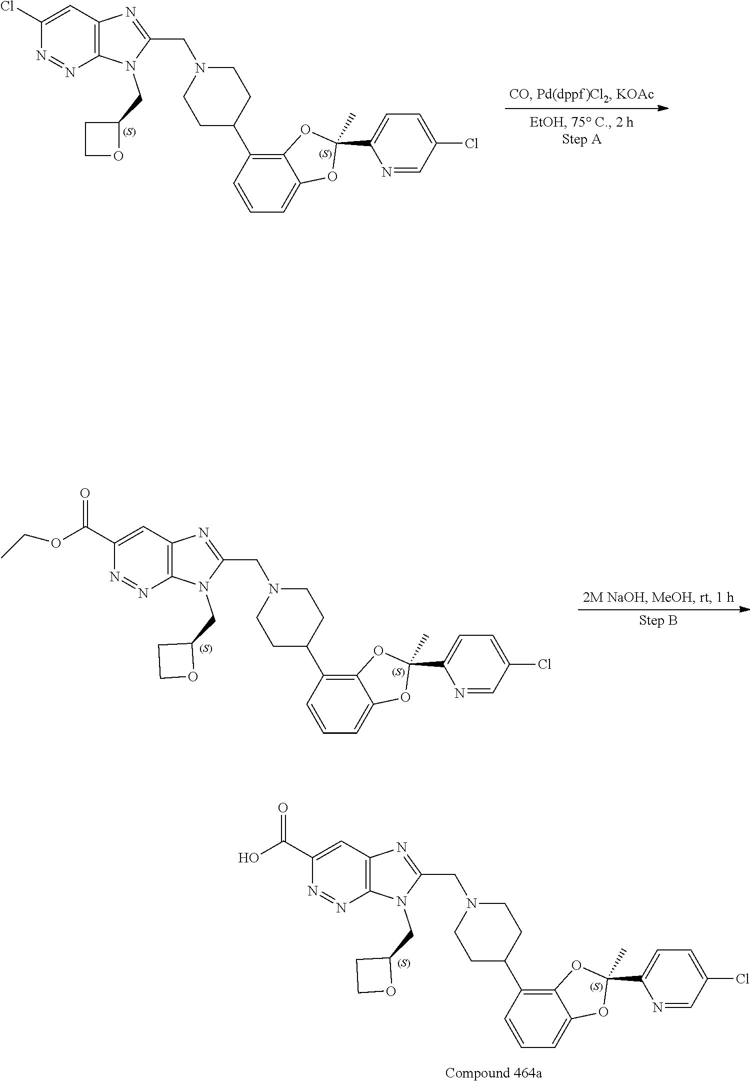

### Step A: ethyl6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate

To a solution of 3-chloro-6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine (100 mg, 0.177 mmol) in EtOH (2 mL) was added Pd(dppf)Cl₂ (26 mg, 0.035 mmol) and KOAc (52 mg, 0.531 mmol). The resulting mixture was heated to 75° C. and stirred for 2 hours under CO. The reaction was diluted with EtOAc (20 mL), washed with water (15 mL). The organic layers were combined, dried over Na₂SO₄, filtered and concentrated under vacuum. The residue was applied on a silica gel column and eluted with EtOAc to give ethyl 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate (61 mg, 60% yield) as a white solid. MS Calcd.: 604.2; MS Found: 605.2 [M+H]⁺.

### Step B: 6-({4-[(2S)-2-(5-chloropyridin-2-yl)-2-methyl-2H-1,3-benzodioxol-4-yl]piperidin-1-yl}methyl)-7-{[(2S)-oxetan-2-yl]methyl}-7H-imidazo[4,5-c]pyridazine-3-carboxylic acid (Compound 464a)

To a solution of ethyl 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carboxylate (61 mg, 0.099 mmol) in MeOH (1.5 mL) and NaOH (2 M, 2 mL) was stirred at room temperature for 1 hour. The solvent was removed in vacuo. The residue was purified by Prep-HPLC to afford 6-({4-[(2S)-2-(5-chloropyridin-2-yl)-2-methyl-2H-1,3-benzodioxol-4-yl]piperidin-1-yl}methyl)-7-{[(2S)-oxetan-2-yl]methyl}-7H-imidazo[4,5-c]pyridazine-3-carboxylic acid (Compound 464a) (15.44 mg, yield: 27.1%) as a white solid.

MS Calcd.: 576.2; MS Found: 577.1 [M+H]⁺.

¹H NMR (400 MHz, DMSO-d₆): δ 8.72 (d, J=2.4 Hz, 1H), 8.41 (s, 1H), 8.00 (dd, J=8.4 Hz, 2.4 Hz, 1H), 7.60 (d, J=8.4 Hz, 1H), 6.73-6.83 (m, 3H), 5.21-5.30 (m, 1H), 4.97-5.03 (m, 1H), 4.82-4.90 (m, 1H), 4.43-4.51 (m, 1H), 4.33-4.41 (m, 1H), 3.97-4.08 (m, 2H), 2.93-3.05 (m, 2H), 2.60-2.76 (m, 2H), 2.45-2.57 (m, 1H), 2.23-2.35 (m, 2H), 2.01 (s, 3H), 1.70-1.88 (m, 4H).

### Example 269: 4-[3-(4-chloro-2-fluorophenyl)-2,3-dihydro-1,4-benzodioxin-5-yl]-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 465a)

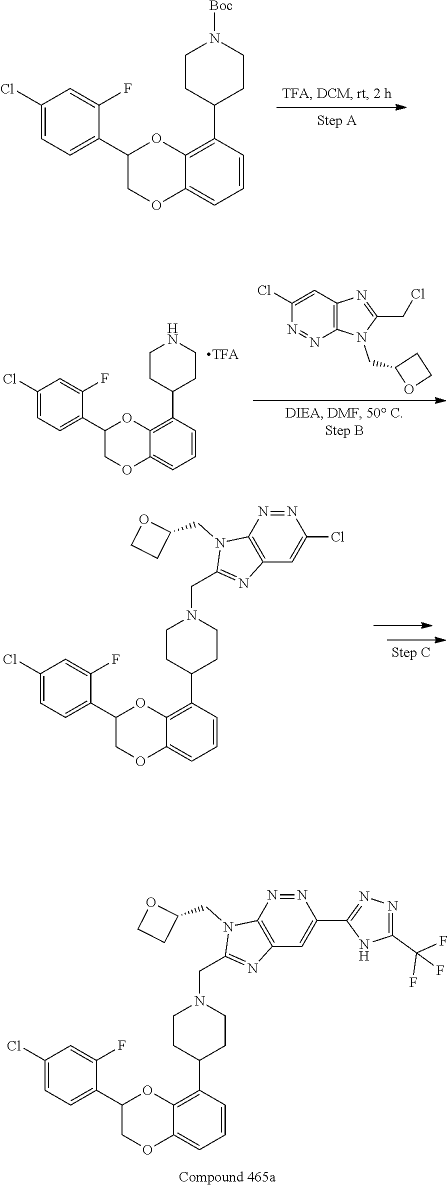 [SMILES: CC(C)(C)OC(=O)N1CCC(c2cccc3c2OC(c2ccc(Cl)cc2F)CO3)CC1 ; Fc1cc(Cl)ccc1C1COc2cccc(C3CCNCC3)c2O1 ; ClCc1nc2cc(Cl)nnc2n1C[C@@H]1CCO1 ; Fc1cc(Cl)ccc1C1COc2cccc(C3CCN(Cc4nc5cc(Cl)nnc5n4C[C@@H]4CCO4)CC3)c2O1 ; Fc1cc(Cl)ccc1C1COc2cccc(C3CCN(Cc4nc5cc(-c6nnc(C(F)(F)F)[nH]6)nnc5n4C[C@@H]4CCO4)CC3)c2O1]

### Step A: 4-(3-(4-chloro-2-fluorophenyl)-2,3-dihydrobenzo[b][1,4]dioxin-5-yl)piperidine. TFA salt

A solution of tert-butyl 4-(3-(4-chloro-2-fluorophenyl)-2,3-dihydrobenzo[b][1,4]dioxin-5-yl)piperidine-1-carboxylate (174 mg, 0.39 mmol), TFA (1 mL) in DCM (4 mL) was stirred at room temperature for 2 h. After the reaction was completed, the reaction was concentrated to give 4-(3-(4-chloro-2-fluorophenyl)-2,3-dihydrobenzo[b][1,4]dioxin-5-yl)piperidine. TFA salt (202 mg, crude) as yellow oil.

MS Calcd.: 347.1; MS Found: 348.3 [M+H]⁺.

### Step B: 3-chloro-6-((4-(3-(4-chloro-2-fluorophenyl)-2,3-dihydrobenzo[b][1,4]dioxin-5-yl)piperidin-1-yl)methyl)-7-(((S)-oxetan-2-yl)methyl)-7H-imidazo[4,5-c]pyridazine

A mixture of 4-(3-(4-chloro-2-fluorophenyl)-2,3-dihydrobenzo[b][1,4]dioxin-5-yl)piperidine. TFA salt (crude 202 mg), (S)-3-chloro-6-(chloromethyl)-7-(oxetan-2-ylmethyl)-7H-imidazo[4,5-c]pyridazine (100 mg, 0.37 mmol), DIEA (143 mg, 1.11 mmol) in DMF (2 mL) was stirred at 50° C. for 5 hours. After the reaction was completed, the mixture was diluted with EA (30 mL), washed with H₂O (20 mL*2). The combined organic layer was dried over anhydrous sodium sulfate, filtered and evaporated to dryness. The residue was purified by Prep-TLC (EA/PE=2/1) to give 3-chloro-6-((4-(3-(4-chloro-2-fluorophenyl)-2,3-dihydrobenzo[b][1,4]dioxin-5-yl)piperidin-1-yl)methyl)-7-(((S)-oxetan-2-yl)methyl)-7H-imidazo[4,5-c]pyridazine (120 mg, yield: 55.6%) as colorless oil. MS Calcd.: 583.2; MS Found: 584.3 [M+H]⁺.

### Step C: 4-[3-(4-chloro-2-fluorophenyl)-2,3-dihydro-1,4-benzodioxin-5-yl]-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 465a)

4-[3-(4-chloro-2-fluorophenyl)-2,3-dihydro-1,4-benzodioxin-5-yl]-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 465a) (6.9 mg) as a white solid.

MS Calcd.: 684.2; MS Found: 685.1 [M+H]⁺.

¹H NMR (400 MHz, DMSO-d₆): δ 8.56 (s, 1H), 7.50-7.56 (m, 1H), 7.32 (d, J=8.8 Hz, 2H), 6.73-6.85 (m, 3H), 5.32-5.45 (m, 2H), 5.10-5.17 (m, 1H), 4.91-4.97 (m, 1H), 4.60-4.70 (m, 1H), 4.38-4.52 (m, 2H), 4.00-4.20 (m, 3H), 2.90-3.15 (m, 3 H), 2.75-2.90 (m, 1H), 2.52-2.63 (m, 1H), 2.30-2.45 (m, 2H), 1.78-1.95 (m, 4H). ¹⁹F NMR (377 MHz): −67.44, −118.62, −118.63.

### Example 270: 3-[2-({4-[(2S)-2-(5-chloropyridin-2-yl)-2-methyl-2H-1,3-benzodioxol-4-yl]piperidin-1-yl}methyl)-3-[(2R)-2-methoxypropyl]-3H-imidazo[4,5-c]pyridin-6-yl]-4,5-dihydro-1,2,4-oxadiazol-5-one (Compound 466a)

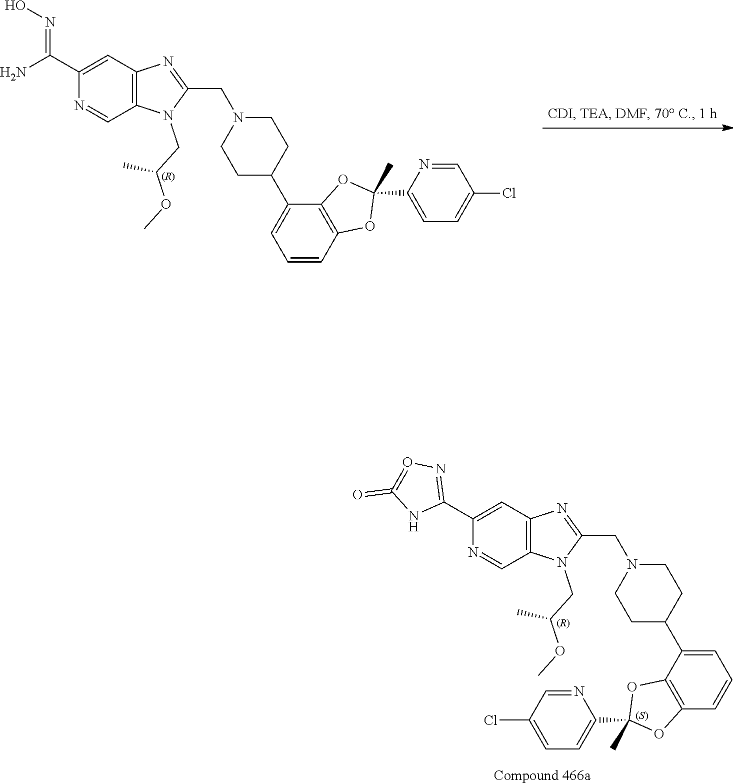

2-((4-((R)-2-(5-chloropyridin-2-yl)-2-methylbenzo[d][1,3]dioxol-4-yl)piperidin-1-yl)methyl)-N′-hydroxy-3-((R)-2-methoxypropyl)-3H-imidazo[4,5-c]pyridine-6-carboximidamide (100 mg) in DMF (2.0 mL) was added TEA (51 mg, 0.51 mmol) and CDI (41 mg, 0.25 mmol). The resulting mixture was stirred at 70° C. for 1 h. After the reaction was completed, the mixture was purified directly by Prep-HPLC (0.1% FA/H₂O/CH₃CN) to give 3-[2-({4-[(2S)-2-(5-chloropyridin-2-yl)-2-methyl-2H-1,3-benzodioxol-4-yl]piperidin-1-yl}methyl)-3-[(2R)-2-methoxypropyl]-3H-imidazo[4,5-c]pyridin-6-yl]-4,5-dihydro-1,2,4-oxadiazol-5-one (Compound 466a) (50 mg, yield: 47%) as a white solid.

MS Calcd.: 617.2; MS Found: 618.1 [M+H]⁺.

¹H NMR (400 MHz, DMSO-d₆): δ 9.05 (s, 1H), 8.71 (s, 1H), 8.15 (s, 1H), 7.99 (d, J=7.6 Hz, 1H), 7.59 (d, J=8.4 Hz, 1H), 6.70-6.83 (m, 3H), 4.40-4.57 (m, 2H), 4.06 (d, J=13.6 Hz, 1H), 3.85-3.94 (m, 1H), 3.76 (d, J=13.6 Hz, 1H), 3.00-3.10 (m, 4H), 2.78-2.86 (m, 1H), 2.58-2.70 (m, 1H), 2.24-2.35 (m, 1H), 2.11-2.23 (m, 1H), 2.01 (s, 3H), 1.68-1.89 (m, 4H), 1.20 (d, J=5.6 Hz, 3H).

### Example 271: 2-chloro-5-(2-methyl-4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}-2H-1,3-benzodioxol-2-yl)pyridine (Compound 467a)

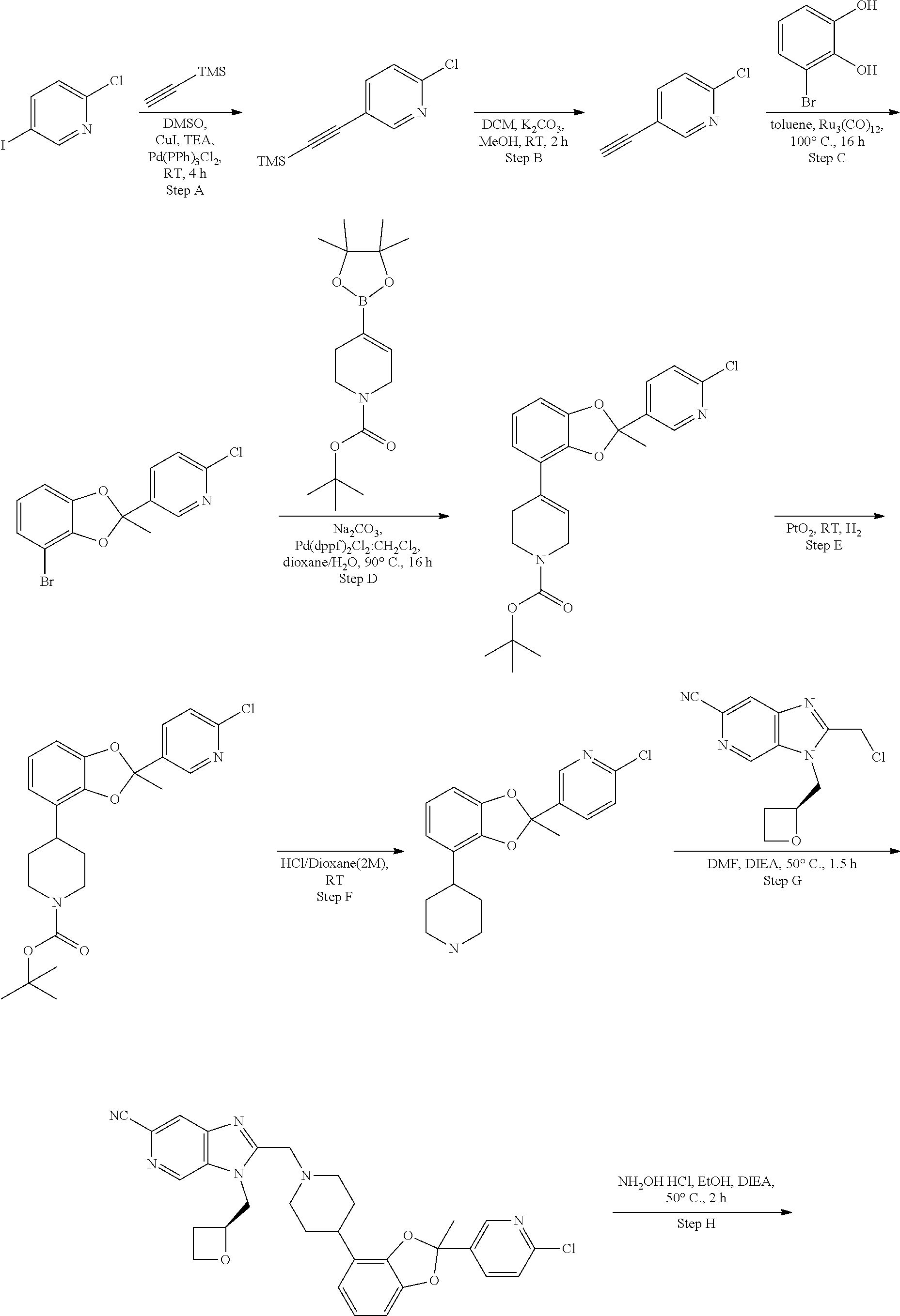

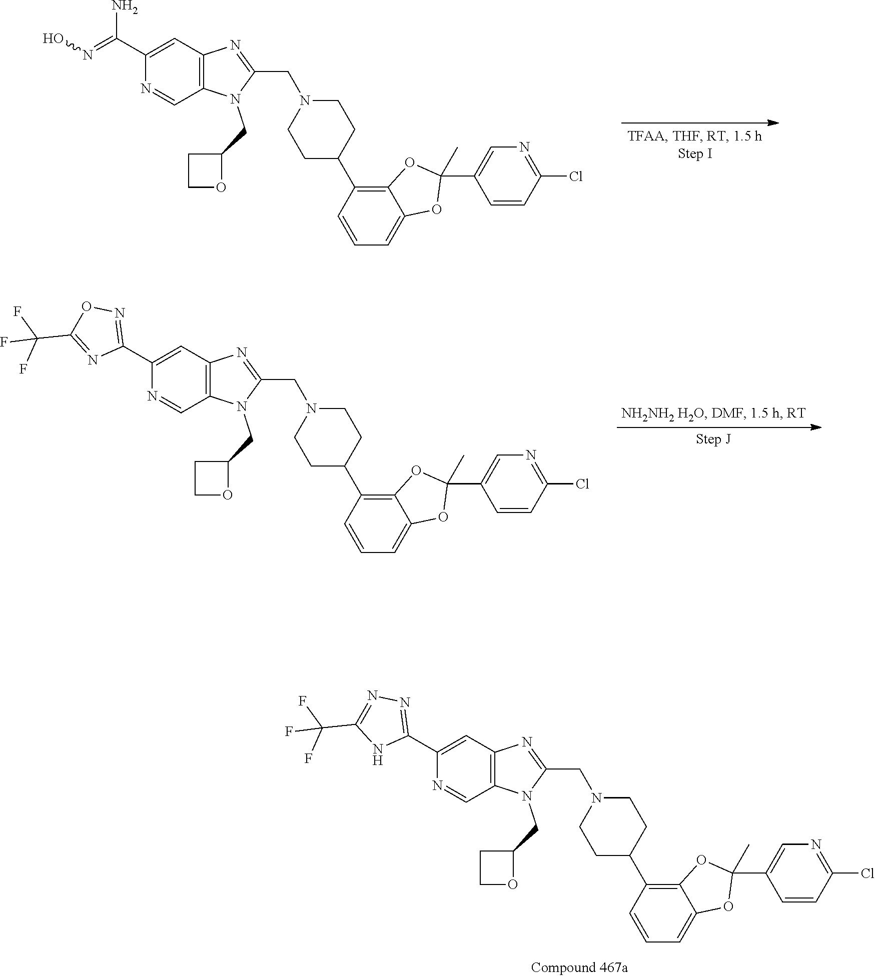 [SMILES: CC1(c2ccc(Cl)nc2)Oc2cccc(C3CCN(Cc4nc5cc(C(N)=NO)ncc5n4C[C@@H]4CCO4)CC3)c2O1 ; CC1(c2ccc(Cl)nc2)Oc2cccc(C3CCN(Cc4nc5cc(-c6noc(C(F)(F)F)n6)ncc5n4C[C@@H]4CCO4)CC3)c2O1 ; CC1(c2ccc(Cl)nc2)Oc2cccc(C3CCN(Cc4nc5cc(-c6nnc(C(F)(F)F)[nH]6)ncc5n4C[C@@H]4CCO4)CC3)c2O1]

### Step A: 2-chloro-5-((trimethylsilyl)ethynyl)pyridine

A mixture of 2-chloro-5-iodopyridine (11.0 g, 46.0 mmol), ethynyltrimethylsilane (8.11 g, 50.6 mmol), CuI (175 mg, 0.92 mmol), TEA (7.25 g, 65.9 mmol) and Pd(PPh)₃Cl₂ (1.3 g, 1.8 mmol) in DMSO (100 mL) was stirred at rt for 4 hours under N₂ atmosphere. After the reaction was completed, the reaction was filtered and diluted with ethyl acetate (200 mL). The organic layer was washed with H₂O (300 mL×4), then dried over sodium sulfate, filtered and concentrated in vacuum. The residue was purified by column chromatography on silica gel (PE:EA=10:1) to give 2-chloro-5-((trimethylsilyl)ethynyl)pyridine (11.0 g, crude). MS Calcd.: 209.0; MS Found: 210.2 [M+H]⁺.

### Step B: 2-chloro-5-ethynylpyridine

A mixture of 2-chloro-5-((trimethylsilyl)ethynyl)pyridine (11.0 g, 53.3 mmol), K₂CO₃ (21.7 g, 157.1 mmol) in DCM (60 mL) and MeOH (60 mL) was stirred at rt for 2 h. After the reaction was completed, the reaction mixture was filtered and the filtrate concentrated. The residue was purified by column chromatography on silica gel (PE:EA=12:1) to give 2-chloro-5-ethynylpyridine (3.2 g, yield: 43%). MS Calcd.: 137.0; MS Found: 138.1 [M+H]⁺.

### Step C: 5-(4-bromo-2-methylbenzo[d][1,3]dioxol-2-yl)-2-chloropyridine

A mixture of 2-chloro-5-ethynylpyridine (2.55 g, 18.6 mmol), 3-bromobenzene-1,2-diol (4.2 g, 22.3 mmol) and Ru₃(CO)₁₂ (594 mg, 0.93 mmol) in toluene (30 mL) was stirred at 100° C. for 16 hours under N₂ atmosphere. After the reaction was completed, the reaction mixture was filtered. The filtrate was diluted with ethyl acetate (80 mL), washed with brine (100 mL×2), dried over sodium sulfate, filtered and concentrated in vacuum. The residue was purified by column chromatography on silica gel (PE:EA=8:1) to give 5-(4-bromo-2-methylbenzo[d][1,3]dioxol-2-yl)-2-chloropyridine (700 mg, yield: 11%). MS Calcd.: 325.0; MS Found: 326.0 [M+H]⁺.

### Step D: tert-butyl 4-(2-(6-chloropyridin-3-yl)-2-methylbenzo[d][1,3]dioxol-4-yl)-3,6-dihydropyridine-1(2H)-carboxylate

A mixture of 5-(4-bromo-2-methylbenzo[d][1,3]dioxol-2-yl)-2-chloropyridine (686 mg, 2.1 mmol), isopropyl 4-(4,4,5,5-tetramethyl-1,3,2-dioxaborolan-2-yl)-3,6-dihydropyridine-1(2H)-carboxylate (583 mg, 1.9 mmol), Na₂CO₃ (664 mg, 6.27 mmol), Pd(dppf)₂Cl₂·CH₂Cl₂ (76 mg, 0.10 mmol) in dioxane (10 mL) and H₂O (1 mL) was stirred at 90° C. for 16 hours under N₂ atmosphere. After the reaction was completed, the mixture was filtered. The filtrate was diluted with EA (70 mL). The organic layer was washed with brine (60 mL×2), then dried over anhydrous sodium sulfate, filtered and evaporated to dryness. The residue was purified by column chromatography on silica gel (PE/EA=5/1) to give tert-butyl 4-(2-(6-chloropyridin-3-yl)-2-methylbenzo[d][1,3]dioxol-4-yl)-3,6-dihydropyridine-1(2H)-carboxylate (500 mg, yield: 58%). MS Calcd.: 428.2; MS Found: 451.1 [M+23]⁺.

### Step E: tert-butyl 4-(2-(6-chloropyridin-3-yl)-2-methylbenzo[d][1,3]dioxol-4-yl)piperidine-1-carboxylate

A mixture of tert-butyl 4-(2-(6-chloropyridin-3-yl)-2-methylbenzo[d][1,3]dioxol-4-yl)-3,6-dihydropyridine-1(2H)-carboxylate (350 mg, 0.82 mmol) and PtO₂ (35 mg, 0.154 mmol) in MeOH (5 mL) was stirred at rt for 3 hours under H₂ (1 atm). The mixture was filtered, and the filtrate was evaporated to dryness. The residue was purified by column chromatography on silica gel (PE/EA=5/1) to give tert-butyl 4-(2-(6-chloropyridin-3-yl)-2-methylbenzo[d][1,3]dioxol-4-yl)piperidine-1-carboxylate (307 mg, yield: 87%). MS Calcd.: 430.2; MS Found: 431.1 [M+H]⁺.

### Step F: 2-chloro-5-(2-methyl-4-(piperidin-4-yl)benzo[d][1,3]dioxol-2-yl)pyridine

A mixture of tert-butyl 4-(2-(6-chloropyridin-3-yl)-2-methylbenzo[d][1,3]dioxol-4-yl)piperidine-1-carboxylate (307 mg, 0.714 mmol) in HCl/Dioxane (4 mL, 2 M) was stirred at rt for 1 hour. The mixture filtered and the filtrate was concentrated to give 2-chloro-5-(2-methyl-4-(piperidin-4-yl)benzo[d][1,3] dioxol-2-yl)pyridine (108 mg, yield: 46%). MS Calcd.: 330.2; MS Found: 331.2 [M+H]⁺.

### Step G: 2-((4-(2-(6-chloropyridin-3-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile

A mixture of (S)-2-(chloromethyl)-3-(oxetan-2-ylmethyl)-3H-imidazo[4,5-c]pyridine-6-carbonitrile (60 mg, 0.23 mmol), 2-chloro-5-(2-methyl-4-(piperidin-4-yl)benzo[d][1,3]dioxol-2-yl)pyridine (76 mg), and DIEA (89 mg, 2.6 mmol) in DMF (3 ml) was stirred at 50° C. for 1.5 hours. After the reaction was completed, the mixture was diluted with ethyl acetate (15 mL), washed with brine (10 mL×2). The organic layer was dried over sodium sulfate, filtered and concentrated in vacuum. The residue was purified by Prep-TLC (DCM/MeOH=10/1) to give 2-((4-(2-(6-chloropyridin-3-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (80 mg, yield: 62%) as a white solid. MS Calcd.: 556.2; MS Found: 557.3 [M+H]⁺.

### Step H: 2-((4-(2-(6-chloropyridin-3-yl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide

A mixture of 2-((4-(2-(6-chloropyridin-3-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (80 mg, 0.144 mmol), hydroxylamine hydrochloride (26 mg, 0.430 mmol) and DIEA (56 mg, 0.430 mmol) in EtOH (3 ml) was stirred at 50° C. for 2 hours. The mixture was diluted with EA (15 mL), washed with brine (15 mL×2). The organic was dried over sodium sulfate, filtered and concentrated under reduced pressure to give 2-((4-(2-(6-chloropyridin-3-yl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (100 mg, crude) as a white solid. MS Calcd.: 589.2; MS Found: 590.3 [M+H]⁺.

### Step I: 3-(2-((4-(2-(6-chloropyridin-3-yl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole

A mixture of 2-((4-(2-(6-chloropyridin-3-yl)-2-methylbenzo[d][1,3]dioxol-4-yl)piperidin-1-yl)methyl)-N′-hydroxy-3-(((S)-oxetan-2-yl)methyl)-3H-imidazo[4,5-c]pyridine-6-carboximidamide (100 mg), TFAA (106 mg, 0.51 mmol) in THE (3 mL) was stirred at RT for 1.5 h. The mixture was diluted with EA (10 mL), washed with sat. NaHCO₃ (6 mL). The organic layer was dried over sodium sulfate, filtered and concentrated under reduced pressure. The residue was purified by Prep-TLC (DCM/MeOH=10/1) to afford 3-(2-((4-(2-(6-chloropyridin-3-yl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole (37 mg, yield: 12%) as yellow oil. MS Calcd.: 667.2; MS Found: 668.3 [M+H]⁺.

### Step J: 2-chloro-5-(2-methyl-4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}-2H-1,3-benzodioxol-2-yl)pyridine (Compound 467a)

A mixture of 3-(2-((4-(2-(6-chloropyridin-3-yl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-5-(trifluoromethyl)-1,2,4-oxadiazole (37 mg, 0.056 mmol) and hydrazine hydrate (8.3 mg, 0.166 mmol) in DMF (3 mL) was stirred at room temperature for 1.5 h. The reaction mixture was purified directly by Prep-HPLC (TFA) to give 2-chloro-5-(2-methyl-4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}-2H-1,3-benzodioxol-2-yl)pyridine (Compound 467a) (4.7 mg, yield: 12%) as a white solid.

MS Calcd.: 666.2; MS Found: 667.1 [M+H]⁺.

¹H NMR (400 MHz, DMSO-d6): δ 9.23-9.28 (m, 1H), 8.60-8.70 (m, 1H), 8.27 (s, 1H), 7.98-8.10 (m, 1H), 7.60 (dd, J=8.4 Hz, 6.4 Hz, 1H), 6.70-6.93 (m, 3H), 5.05-5.17 (m, 1H), 4.87-4.97 (m, 1H), 4.70-4.82 (m, 1H), 4.46-4.56 (m, 1H), 4.38-4.45 (m, 1H), 4.00-4.20 (m, 1H), 3.50-3.82 (m, 3H), 2.80-3.10 (m, 1H), 2.60-2.82 (m, 2H), 2.50-2.58 (m, 1H), 2.30-2.45 (m, 1H), 1.80-2.21 (m, 7H). ¹⁹F NMR (377 MHz): −63.71.

### Example 272: 3-fluoro-4-{[(6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridin-2-yl)oxy]methyl}benzonitrile (Compound 313a)

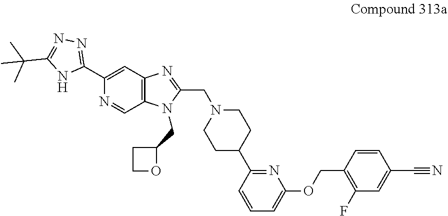 [SMILES: CC(C)(C)c1nnc(-c2cc3nc(CN4CCC(c5cccc(OCc6ccc(C#N)cc6F)n5)CC4)n(C[C@@H]4CCO4)c3cn2)[nH]1]

A mixture of (S)-2-((4-(6-((4-chloro-2-fluorobenzyl)oxy)pyridin-2-yl)piperidin-1-yl)methyl)-3-(oxetan-2-ylmethyl)-6-(5-(trifluoromethyl)-4H-1,2,4-triazol-3-yl)-3H-imidazo[4,5-c]pyridine (60 mg, 0.09 mmol), Zn(CN)₂ (107 mg, 0.91 mmol), RuPhos Pd G₃ (23 mg, 0.027 mmol) and X-Phos (13 mg, 0.027 mmol) in anhydrous NMP (2.0 mL) was stirred at 130° C. for 2 h under Ar. After cooling down to room temperature, the reaction mixture was filtered and the filtrate purified by Prep-HPLC (0.1% FA/H₂O/CH₃CN) to give 3-fluoro-4-{[(6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridin-2-yl)oxy]methyl}benzonitrile (Compound 313a) (9.8 mg, yield: 16%) as a white solid.

MS Calcd.: 647.2; MS Found: 648.1 [M+H]⁺.

¹H NMR (400 MHz, DMSO-d6): δ 9.17 (s, 1H), 8.29 (s, 1H), 7.88 (d, J=10.0 Hz, 1H), 7.68-7.72 (m, 2H), 7.66 (t, J=8.0 Hz, 1H), 6.89 (d, J=7.2 Hz, 1H), 6.73 (d, J=8.0 Hz, 1H), 5.48 (s, 2H), 5.14-5.22 (m, 1H), 4.90-4.98 (m, 1H), 4.73-4.82 (m, 1H), 4.40-4.55 (m, 2H), 4.01 (d, J=14.0 Hz, 1H), 3.86 (d, J=13.6 Hz, 1H), 2.97-3.05 (m, 1H), 2.83-2.90 (m, 1H), 2.68-2.79 (m, 1H), 2.55-2.65 (m, 1H), 2.42-2.53 (m, 1H), 2.15-2.32 (m, 2H), 1.60-1.84 (m, 4H). ¹⁹F NMR (377 MHz): −63.72, −115.56.

### Example 273: 4-[3-(4-chloro-2-fluorophenyl)-2,3-dihydro-1,4-benzodioxin-5-yl]-1-{[6-(5-methyl-4H-1,2,4-triazol-3-yl)-3-{[(2S)-oxetan-2-yl]methyl}-3H-imidazo[4,5-c]pyridin-2-yl]methyl}piperidine (Compound 468a)

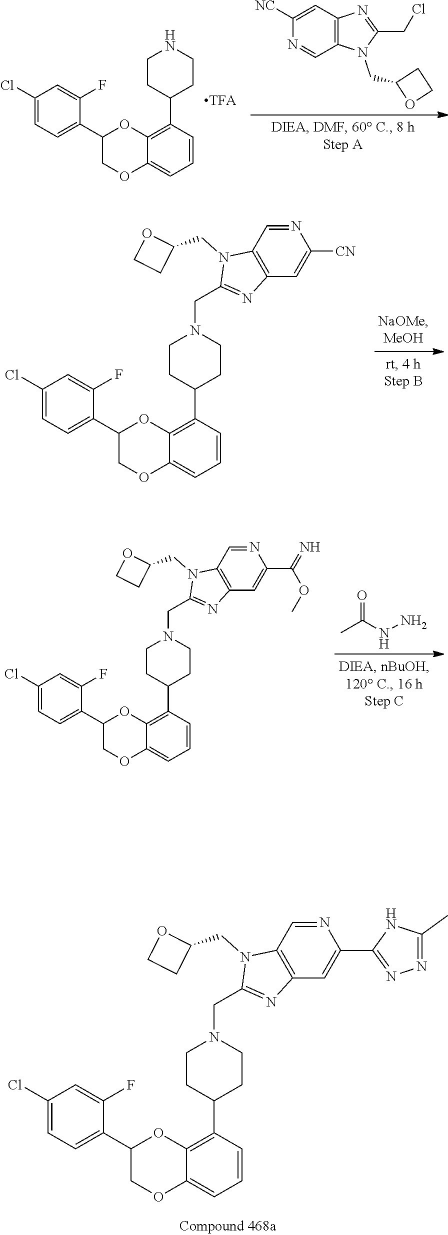

### Step A: 2-((4-(3-(4-chloro-2-fluorophenyl)-2,3-dihydrobenzo[b][1,4]dioxin-5-yl)piperidin-1-yl)methyl)-3-(((S)-oxetan-2-yl)methyl)-3H-imidazo[4,5-c]pyridine-6-carbonitrile

To a solution of 4-(3-(4-chloro-2-fluorophenyl)-2,3-dihydrobenzo[b][1,4]dioxin-5-yl)piperidine TFA salt (crude, 240 mg, 0.4 mmol) in DMF (2 mL) was added DIEA (156 mg, 1.2 mmol). After stirring at rt for 2 mins, (S)-2-(chloromethyl)-3-(oxetan-2-ylmethyl)-3H-imidazo[4,5-c]pyridine-6-carbonitrile (100 mg, 0.4 mmol) was added. The resulting mixture was heated to 60° C. for 8 hours. The reaction mixture was quenched with water (20 mL), extracted with ethyl acetate (30 mL×2). The organic layers were combined, dried over Na₂SO₄, filtered and concentrated under vacuum. The residue was applied on a silica gel column and eluted with PE:EA=3:1 to give 2-((4-(3-(4-chloro-2-fluorophenyl)-2,3-dihydrobenzo[b][1,4]dioxin-5-yl)piperidin-1-yl)methyl)-3-(((S)-oxetan-2-yl)methyl)-3H-imidazo[4,5-c]pyridine-6-carbonitrile (138 mg, 60% yield) as a light yellow oil. MS Calcd.: 573.2; MS Found: 574.2 [M+H]⁺.

### Step B: methyl 2-((4-(3-(4-chloro-2-fluorophenyl)-2,3-dihydrobenzo[b][1,4]dioxin-5-yl)piperidin-1-yl)methyl)-3-(((S)-oxetan-2-yl)methyl)-3H-imidazo[4,5-c]pyridine-6-carbimidate

To a solution of 2-((4-(3-(4-chloro-2-fluorophenyl)-2,3-dihydrobenzo[b][1,4]dioxin-5-yl)piperidin-1-yl)methyl)-3-(((S)-oxetan-2-yl)methyl)-3H-imidazo[4,5-c]pyridine-6-carbonitrile (118 mg, 0.21 mmol) in MeOH (2 mL) was added sodium methanolate (111 mg, 2.1 mmol). The solution was stirred at room temperature for 4 hours. The reaction mixture was quenched with water (10 mL), extracted with ethyl acetate (10 mL×3). The organic layers were combined, dried over Na₂SO₄, filtered and concentrated under vacuum. The residue was purified by silica gel (PE/EA=3/1) to give 2-((4-(3-(4-chloro-2-fluorophenyl)-2,3-dihydrobenzo[b][1,4]dioxin-5-yl)piperidin-1-yl)methyl)-3-(((S)-oxetan-2-yl)methyl)-3H-imidazo[4,5-c]pyridine-6-carbimidate (119 mg, yield: 95%) as a light-yellow solid. MS Calcd.: 605.2; MS Found: 606.3 [M+H]⁺.

### Step C: 4-[3-(4-chloro-2-fluorophenyl)-2,3-dihydro-1,4-benzodioxin-5-yl]-1-{[6-(5-methyl-4H-1,2,4-triazol-3-yl)-3-{[(2S)-oxetan-2-yl]methyl}-3H-imidazo[4,5-c]pyridin-2-yl]methyl}piperidine (Compound 468a)

A mixture of methyl 2-((4-(3-(4-chloro-2-fluorophenyl)-2,3-dihydrobenzo[b][1,4]dioxin-5-yl)piperidin-1-yl)methyl)-3-(((S)-oxetan-2-yl)methyl)-3H-imidazo[4,5-c]pyridine-6-carbimidate (99 mg, 0.163 mmol) in n-BuOH (2 mL) was added acetohydrazide (24.1 mg, 0.326 mmol) and DIEA (63.6 mg, 0.489 mmol). The solution was stirred at 120° C. for 16 hours. The solvent was removed in vacuo. The residue was purified by Prep-HPLC to give 4-[3-(4-chloro-2-fluorophenyl)-2,3-dihydro-1,4-benzodioxin-5-yl]-1-{[6-(5-methyl-4H-1,2,4-triazol-3-yl)-3-{[(2S)-oxetan-2-yl]methyl}-3H-imidazo[4,5-c]pyridin-2-yl]methyl}piperidine (Compound 468a) (10 mg, yield: 8.5%) as a white solid.

MS Calcd.: 629.2; MS Found: 630.3 [M+H]⁺.

¹H NMR (400 MHz, MeOD) δ 9.20 (s, 1H), 8.28 (s, 1H), 7.53-7.60 (m, 2H), 7.40 (dd, J=2.0 Hz, 8.4 Hz, 1H), 6.82-6.94 (m, 3H), 5.49 (dd, J=2.0 Hz, 7.6 Hz, 1H), 5.04-5.12 (m, 1H), 4.80-4.90 (m, 2H), 4.68-4.75 (m, 1H), 4.43-4.54 (m, 2H), 4.32-4.43 (m, 1H), 4.10-4.18 (m, 1H), 3.60-3.80 (m, 2H), 3.10-3.25 (m, 3H), 2.68-2.80 (m, 1H), 2.55-2.60 (m, 1H), 2.34-2.40 (m, 4H), 1.90-2.10 (m, 4H). ¹⁹F NMR (377 MHz): −115.04, −115.06.

### Example 274: 3-fluoro-4-[(2-fluoro-5-{4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazin-1-yl}phenoxy)methyl]benzonitrile (Compound 469a)

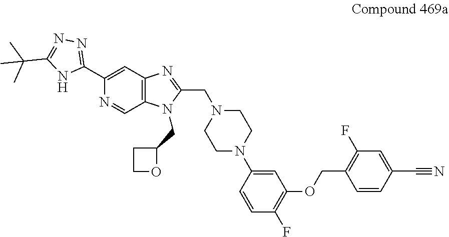 [SMILES: CC(C)(C)c1nnc(-c2cc3nc(CN4CCN(c5ccc(F)c(OCc6ccc(C#N)cc6F)c5)CC4)n(C[C@@H]4CCO4)c3cn2)[nH]1]

3-fluoro-4-[(2-fluoro-5-{4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazin-1-yl}phenoxy)methyl]benzonitrile (Compound 469a) (15 mg) as a white solid.

MS Calcd.: 665.2; MS Found: 666.1 [M+H]⁺.

¹H NMR (400 MHz, DMSO-d6): δ 9.16 (s, 1H), 8.26-8.32 (m, 1H), 7.91 (d, J=10.0 Hz, 1H), 7.73-7.80 (m, 2H), 7.02-7.11 (m, 1H), 6.86 (dd, J=7.2 Hz, 2.4 Hz, 1H), 6.46-6.52 (m, 1H), 5.30 (s, 2H), 5.10-5.22 (m, 1H), 4.85-4.96 (m, 1H), 4.70-4.80 (m, 1H), 4.46-4.55 (m, 1H), 4.39-4.46 (m, 1H), 4.05 (d, J=13.6 Hz, 1H), 3.90 (d, J=13.6 Hz, 1H), 3.06-3.14 (m, 4H), 2.59-2.80 (m, 6H). ¹⁹F NMR (377 MHz): −63.42, −115.12, −146.11.

### Example 275: 4-{3-[(4-chloro-2-fluorophenyl)methoxy]-4-fluorophenyl}-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 470a)

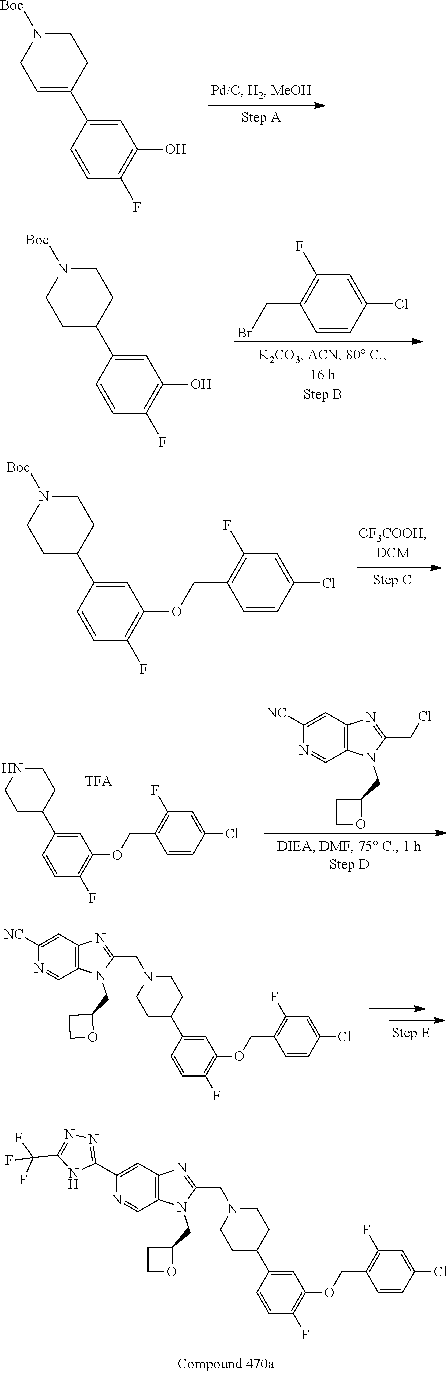

### Step A: tert-butyl 4-(4-fluoro-3-hydroxyphenyl)piperidine-1-carboxylate

A mixture of tert-butyl 4-(4-fluoro-3-hydroxyphenyl)-3,6-dihydropyridine-1(2H)-carboxylate (1.0 g, 3.4 mmol) and Pd/C (10%, 100 mg) in MeOH (20 mL) was stirred at rt for 1 hour under H₂ (1 atm). After the reaction was completed, the mixture was filtered and the filtrate was concentrated to give tert-butyl 4-(4-fluoro-3-hydroxyphenyl)piperidine-1-carboxylate (900 mg, 89% yield) as a white solid. MS Calcd.: 295.2; MS Found: 280.9[M-56+41+H]⁺.

### Step B: tert-butyl 4-(3-((4-chloro-2-fluorobenzyl)oxy)-4-fluorophenyl)piperidine-1-carboxylate

A mixture of tert-butyl 4-(4-fluoro-3-hydroxyphenyl)piperidine-1-carboxylate (200 mg, 0.68 mmol), 1-(bromomethyl)-4-chloro-2-fluorobenzene (166 mg, 0.75 mmol) and K₂CO₃ (280 mg, 2.0 mmol) in ACN (10 mL) was stirred at 80° C. for 16 h. After the reaction was completed, the mixture was diluted with H₂O (10 mL) and extracted with EA (20 mL×3). The organic layer was dried over anhydrous sodium sulfate, filtered and evaporated to dryness. The residue was purified by column chromatography on silica gel (EA/PE=1/10) to give tert-butyl 4-(3-((4-chloro-2-fluorobenzyl)oxy)-4-fluorophenyl)piperidine-1-carboxylate (200 mg, yield: 67%) as a colorless oil. MS Calcd.: 437.2; MS Found: 423.1[M-56+41+H]⁺.

### Step C: 4-(3-((4-chloro-2-fluorobenzyl)oxy)-4-fluorophenyl)piperidine TFA salt

A solution of tert-butyl 4-(3-((4-chloro-2-fluorobenzyl)oxy)-4-fluorophenyl)piperidine-1-carboxylate (400 mg, 0.91 mmol) and TFA (0.5 mL) in DCM (2 mL) was stirred at room temperature for 1 h. Upon completion, the reaction mixture was concentrated to give 4-(3-((4-chloro-2-fluorobenzyl)oxy)-4-fluorophenyl)piperidine TFA salt (450 mg, crude) as yellow oil. MS Calcd.: 337.1; MS Found: 337.9 [M+H]⁺.

### Step D: (S)-2-((4-(3-((4-chloro-2-fluorobenzyl)oxy)-4-fluorophenyl)piperidin-1-yl)methyl)-3-(oxetan-2-ylmethyl)-3H-imidazo[4,5-c]pyridine-6-carbonitrile

A mixture of 4-(3-((4-chloro-2-fluorobenzyl)oxy)-4-fluorophenyl)piperidine TFA salt (250 mg, crude), (S)-2-(chloromethyl)-3-(oxetan-2-ylmethyl)-3H-imidazo[4,5-c]pyridine-6-carbonitrile (239 mg, 0.91 mmol), DIEA (263 mg, 2.0 mmol) in DMF (3 mL) was stirred at 75° C. for 1 hour. After the reaction was completed, the mixture was diluted with H₂O (10 mL) and extracted with EA (20 mL×3). The combined organic layer was washed by brine (15 mL×3) and then dried over anhydrous sodium sulfate, filtered and evaporated to dryness. The residue was purified by column chromatography on silica gel (DCM/MeOH=10/1) to give (S)-2-((4-(3-((4-chloro-2-fluorobenzyl)oxy)-4-fluorophenyl)piperidin-1-yl)methyl)-3-(oxetan-2-ylmethyl)-3H-imidazo[4,5-c]pyridine-6-carbonitrile (386 mg, 75% yield) as a yellow solid. MS Calcd.: 563.2; MS Found: 564.4 [M+H]⁺.

### Step E: 4-{3-[(4-chloro-2-fluorophenyl)methoxy]-4-fluorophenyl}-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 470a)

4-{3-[(4-chloro-2-fluorophenyl)methoxy]-4-fluorophenyl}-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 470a) (100 mg) as a white solid.

MS Calcd.: 673.2; MS Found: 674.3 [M+H]⁺.

¹H NMR (400 MHz, DMSO-d6): δ 9.17 (s, 1H), 8.29 (s, 1H), 7.59 (t, J=8.0 Hz, 1H), 7.50 (dd, J=2.0, J=10.0 Hz, 1H), 7.35 (dd, J=2.0 Hz, J=8.4 Hz, 1H), 7.09-7.20 (m, 2H), 6.80-6.89 (m, 1H), 5.10-5.23 (m, 3H), 4.90-4.98 (m, 1H), 4.75-4.80 (m, 1H), 4.49-4.56 (m, 1H), 4.38-4.46 (m, 1H), 3.98-4.03 (m, 1H), 3.84-3.90 (m, 1H), 2.98-3.08 (m, 1H), 2.85-2.95 (m, 1H), 2.70-2.82 (m, 1H), 2.40-2.60 (m, 2H), 2.15-2.33 (m, 2H), 1.55-1.83 (m, 4H). ¹⁹F NMR (377 MHz): −63.71, −115.06, −138.20.

### Example 276: 3-fluoro-4-[(2-fluoro-5-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}phenoxy)methyl]benzonitrile (Compound 471a)

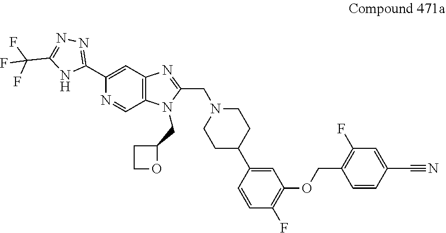 [SMILES: N#Cc1ccc(COc2cc(C3CCN(Cc4nc5cc(-c6nnc(C(F)(F)F)[nH]6)ncc5n4C[C@@H]4CCO4)CC3)ccc2F)c(F)c1]

3-fluoro-4-[(2-fluoro-5-f{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}phenoxy)methyl]benzonitrile (Compound 471a) (5.0 mg) as a white solid.

MS Calcd.: 664.2; MS Found: 665.1 [M+H]⁺.

¹H NMR (400 MHz, DMSO-d6): δ 9.12 (s, 1H), 8.42 (s, 1H), 7.75 (t, J=7.2 Hz, 1H), 7.60 (d, J=8.4 Hz, 2H), 6.99-7.08 (m, 2H), 6.82-6.89 (m, 1H), 5.24-5.35 (m, 3H), 4.94-5.01 (m, 1H), 4.76-4.85 (m, 1H), 4.63-4.72 (m, 1H), 4.45-4.54 (m, 1H), 4.05 (d, J=13.6 Hz, 1H), 3.93 (d, J=13.6 Hz, 1H), 3.02-3.11 (m, 1H), 2.92-2.99 (m, 1H), 2.80-2.90 (m, 1H), 2.50-2.62 (m, 2H), 2.25-2.40 (m, 2H), 1.65-1.88 (m, 4H). ¹⁹F NMR (377 MHz): −66.64, −117.34, −139.52.

### Example 277: 2-[(4-chloro-2-fluorophenyl)methoxy]-3-fluoro-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 472a)

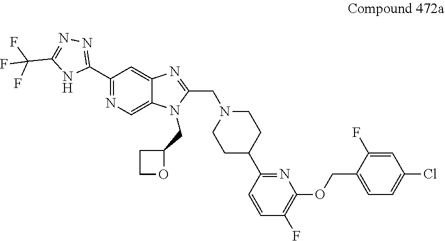 [SMILES: Fc1cc(Cl)ccc1COc1nc(C2CCN(Cc3nc4cc(-c5nnc(C(F)(F)F)[nH]5)ncc4n3C[C@@H]3CCO3)CC2)ccc1F]

2-[(4-chloro-2-fluorophenyl)methoxy]-3-fluoro-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 472a) (12 mg) as a white solid.

MS Calcd.: 674.2; MS Found: 675.0 [M+H]⁺.

¹H NMR (400 MHz, DMSO-d6): δ 9.16 (s, 1H), 8.28 (s, 1H), 7.55-7.63 (m, 2H), 7.48 (dd, J=10.0 Hz, 2.0 Hz, 1H), 7.32 (dd, J=8.0 Hz, 1.6 Hz, 1H), 6.90 (dd, J=8.4 Hz, 2.8 Hz, 1H), 5.47 (s, 2H), 5.14-5.23 (m, 1H), 4.88-4.97 (m, 1H), 4.73-4.81 (m, 1H), 4.39-4.54 (m, 2H), 4.01 (d, J=13.6 Hz, 1H), 3.86 (d, J=13.6 Hz, 1H), 2.96-3.05 (m, 1H), 2.83-2.91 (m, 1H), 2.69-2.80 (m, 1H), 2.57-2.68 (m, 1H), 2.41-2.53 (m, 1H), 2.16-2.35 (m, 2H), 1.60-1.86 (m, 4H). ¹⁹F NMR (377 MHz): −63.52, −115.06, −144.00.

### Example 278: 5-chloro-2-[(2S)-4-[1-({7-[(2R)-2-methoxypropyl]-3-[5-(trifluoromethyl)-4H-1,2,4-triazol-3-yl]-7H-imidazo[4,5-c]pyridazin-6-yl}methyl)piperidin-4-yl]-2-methyl-2H-1,3-benzodioxol-2-yl]pyridine (Compound 473a)

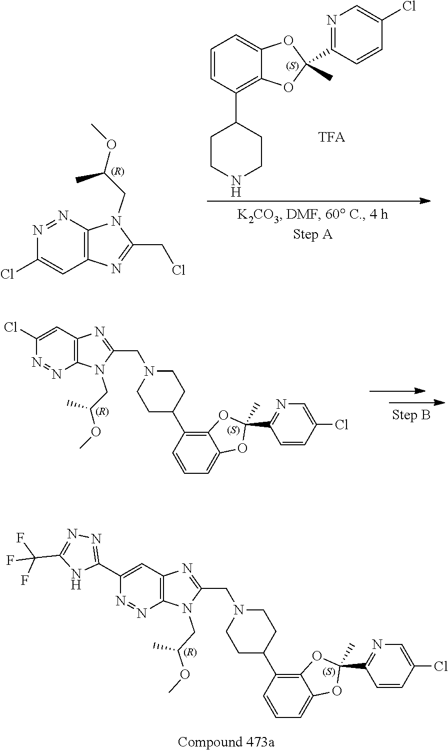

### Step A: 3-chloro-6-((4-((S)-2-(5-chloropyridin-2-yl)-2-methylbenzo[d][1,3]dioxol-4-yl)piperidin-1-yl)methyl)-7-((R)-2-methoxypropyl)-7H-imidazo[4,5-c]pyridazine

A mixture of (S)-5-chloro-2-(2-methyl-4-(piperidin-4-yl)benzo[d][1,3]dioxol-2-yl)pyridine TFA salt (437 mg, 1.32 mmol), (R)-3-chloro-6-(chloromethyl)-7-(2-methoxypropyl)-7H-imidazo[4,5-c]pyridazine (330 mg, 1.2 mmol), K₂CO₃ (662 mg, 4.8 mmol) in DMF (5.0 mL) was stirred at 60° C. for 4 hours. After the reaction was completed, the mixture was diluted with H₂O (40 mL), extracted with EA (30 mL×2). The combined organic layer was dried over anhydrous sodium sulfate, filtered and evaporated to dryness. The residue was purified by column chromatography on silica gel (DCM/MeOH=80/1) to give 3-chloro-6-((4-((S)-2-(5-chloropyridin-2-yl)-2-methylbenzo[d][1,3]dioxol-4-yl)piperidin-1-yl)methyl)-7-((R)-2-methoxypropyl)-7H-imidazo[4,5-c]pyridazine (460 mg, yield: 67%) as a yellow solid. MS Calcd.: 568.2; MS Found: 569.2 [M+H]⁺.

### Step B: 5-chloro-2-[(2S)-4-[1-({7-[(2R)-2-methoxypropyl]-3-[5-(trifluoromethyl)-4H-1,2,4-triazol-3-yl]-7H-imidazo[4,5-c]pyridazin-6-yl}methyl)piperidin-4-yl]-2-methyl-2H-1,3-benzodioxol-2-yl]pyridine (Compound 473a)

5-chloro-2-[(2S)-4-[1-({7-[(2R)-2-methoxypropyl]-3-[5-(trifluoromethyl)-4H-1,2,4-triazol-3-yl]-7H-imidazo[4,5-c]pyridazin-6-yl}methyl)piperidin-4-yl]-2-methyl-2H-1,3-benzodioxol-2-yl]pyridine (Compound 473a) (42 mg) as a yellow solid.

MS Calcd.: 669.2; MS Found: 670.2 [M+H]⁺.

¹H NMR (400 MHz, DMSO-d6): δ 8.71 (s, 1H), 8.50 (s, 1H), 7.99 (d, J=8.4 Hz, 1H), 7.59 (d, J=8.0 Hz, 1H), 6.72-6.83 (m, 3H), 4.68-4.78 (m, 1H), 4.57-4.65 (m, 1 H), 4.03-4.12 (m, 2H), 3.92-3.98 (m, 1H), 3.16 (s, 3H), 2.95-3.05 (m, 2H), 2.60-2.71 (m, 1H), 2.23-2.37 (m, 2H), 2.01 (s, 3H), 1.72-1.90 (m, 4H), 1.20 (d, J=6.0 Hz, 3H). ¹⁹F NMR (377 MHz): −63.65.

### Example 279: 5-chloro-2-[(2S)-2-methyl-4-{1-[(7-{[(3R)-oxolan-3-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]pyridine (Compound 474a)

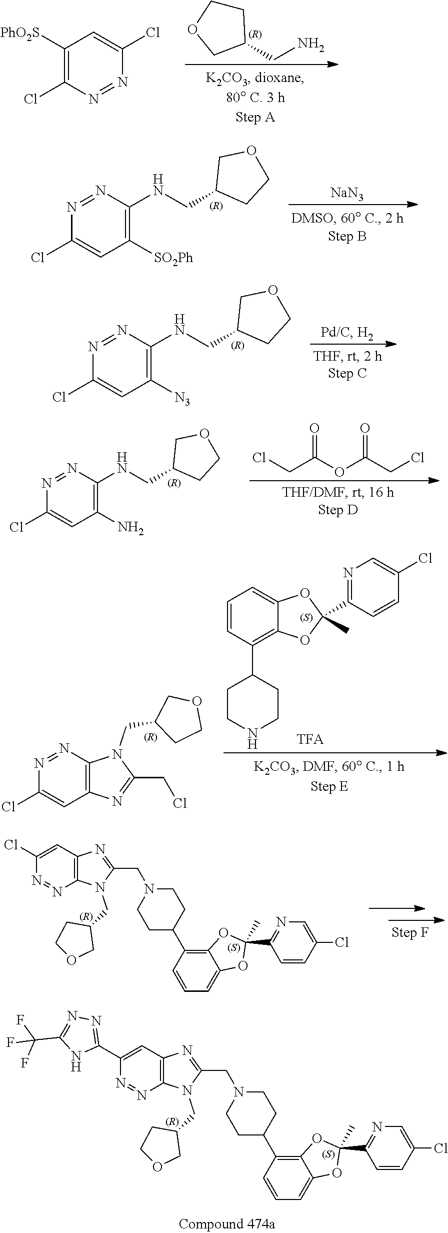

### Step A: (R)-6-chloro-4-(phenylsulfonyl)-N-((tetrahydrofuran-3-yl)methyl)pyridazin-3-amine

A mixture of 3,6-dichloro-4-(phenylsulfonyl)pyridazine (1.8 g, 6.2 mmol), (R)-(tetrahydrofuran-3-yl)methanamine (574 mg, 5.7 mmol), K₂CO₃ (2.4 g, 17.1 mmol) in dioxane (50 mL) was stirred at 80° C. for 3 hours. After the reaction was completed, the reaction mixture was filtered, and the filtrate evaporated to dryness. The residue was purified by column chromatography on silica gel (PE/EA=2/1) to give (R)-6-chloro-4-(phenylsulfonyl)-N-((tetrahydrofuran-3-yl)methyl)pyridazin-3-amine (1.6 g, yield: 80%) as yellow oil. MS Calcd.: 353.1; MS Found: 354.2 [M+H]⁺.

### Step B: (R)-4-azido-6-chloro-N-((tetrahydrofuran-3-yl)methyl)pyridazin-3-amine

To a mixture of (R)-6-chloro-4-(phenylsulfonyl)-N-((tetrahydrofuran-3-yl)methyl)pyridazin-3-amine (1.5 g, 4.3 mmol) in DMSO (30 mL) was added NaN₃ (1.1 g, 17.0 mmol) and the mixture stirred at 60° C. for 2 hours. After the reaction was completed, the mixture was quenched with H₂O (50 mL) and extracted with EA (100 mL*2). The extracts were wash with brine (50 mL*3). The organic layer was dried over anhydrous sodium sulfate, filtered and evaporated to dryness. The residue was purified by column chromatography on silica gel (PE/EA=1/2) to give (R)-4-azido-6-chloro-N-((tetrahydrofuran-3-yl)methyl)pyridazin-3-amine (900 mg, yield: 83%) as yellow oil. MS Calcd.: 254.1; MS Found: 255.2 [M+H]⁺.

### Step C: (R)-6-chloro-N3-((tetrahydrofuran-3-yl)methyl)pyridazine-3,4-diamine

To a mixture of (R)-4-azido-6-chloro-N-((tetrahydrofuran-3-yl)methyl)pyridazin-3-amine (800 mg, 3.15 mmol) in THF (15.0 mL) was added Pd/C (160 mg, 20% on Carbon, wetted with ca. 55% water). The mixture was stirred at rt for 2 hours under H₂ (1 atm). The reaction mixture was filtered through a celite pad. The filtrate was evaporated to give (R)-6-chloro-N3-((tetrahydrofuran-3-yl)methyl)pyridazine-3,4-diamine (760 mg crude, yield: 100%) as a yellow solid. MS Calcd.: 228.1; MS Found: 229.1 [M+H]⁺.

### Step D: (R)-3-chloro-6-(chloromethyl)-7-((tetrahydrofuran-3-yl)methyl)-7H-imidazo[4,5-c]pyridazine

To a mixture of (R)-6-chloro-N3-((tetrahydrofuran-3-yl)methyl)pyridazine-3,4-diamine (730 mg, 3.2 mmol) in THE (10.0 mL)/DMF (5.0 mL) was added 2-chloroacetic anhydride (821 mg, 4.8 mmol). The mixture was stirred at rt for 16 hours. After the reaction was completed, the mixture was quenched with NaHCO₃ (10.0 mL) and extracted with EA (30 mL*2), the organic layer was dried over anhydrous sodium sulfate, filtered and evaporated to dryness. The residue purified by column chromatography on silica gel (PE/EA=1/1) to give (R)-3-chloro-6-(chloromethyl)-7-((tetrahydrofuran-3-yl)methyl)-7H-imidazo[4,5-c]pyridazine (810 mg, yield: 88%) as yellow oil. MS Calcd.: 286.0; MS Found: 287.0 [M+H]⁺.

### Step E: 3-chloro-6-((4-((S)-2-(5-chloropyridin-2-yl)-2-methylbenzo[d][1,3]dioxol-4-yl)piperidin-1-yl)methyl)-7-(((R)-tetrahydrofuran-3-yl)methyl)-7H-imidazo[4,5-c]pyridazine

To a mixture of (S)-5-chloro-2-(2-methyl-4-(piperidin-4-yl)benzo[d][1,3]dioxol-2-yl)pyridine TFA Salt (417 mg, 1.26 mmol) in DMF (6.0 mL) was added K₂CO₃ (434 mg, 3.15 mmol) and (R)-3-chloro-6-(chloromethyl)-7-((tetrahydrofuran-3-yl)methyl)-7H-imidazo[4,5-c]pyridazine (300 mg, 1.05 mmol). The mixture was stirred at 60° C. for 1 hour. After the reaction was completed, the reaction was filtered, and the filtrate evaporated to dryness. The residue was purified by column chromatography on silica gel (PE/EA=1/1) to give 3-chloro-6-((4-((S)-2-(5-chloropyridin-2-yl)-2-methylbenzo[d][1,3]dioxol-4-yl)piperidin-1-yl)methyl)-7-(((R)-tetrahydrofuran-3-yl)methyl)-7H-imidazo[4,5-c]pyridazine (490 mg, yield: 80%) as yellow oil. MS Calcd.: 580.2; MS Found: 581.2 [M+H]⁺.

### Step F: 5-chloro-2-[(2S)-2-methyl-4-{1-[(7-{[(3R)-oxolan-3-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]pyridine (Compound 474a)

5-chloro-2-[(2S)-2-methyl-4-{1-[(7-{[(3R)-oxolan-3-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]pyridine (Compound 474a) (41 mg) as a white solid.

MS Calcd.: 681.2; MS Found: 682.2 [M+H]⁺.

¹H NMR (400 MHz, DMSO-d6): δ 8.74 (d, J=2.4 Hz, 1H), 8.60 (s, 1H), 8.02 (dd, J=8.4 Hz, 2.4 Hz, 1H), 7.64 (d, J=8.4 Hz, 1H), 6.76-6.90 (m, 3H), 4.65-5.15 (m, 1H), 4.57 (d, J=7.6 Hz, 2H), 3.86-3.95 (m, 1H), 3.60-3.75 (m, 7H), 2.80-3.10 (m, 3H), 1.90-2.20 (m, 8H), 1.73-1.84 (m, 1H). ¹⁹F NMR (377 MHz, DMSO-d6): δ −63.67.

### Example 280: 4-[(2S)-2-(4-chloro-2-fluorophenyl)-2-methyl-2H-1,3-benzodioxol-4-yl]-1-[(7-{[(3R)-oxolan-3-yl]methyl}-3-[5-(trifluoromethyl)-4H-1,2,4-triazol-3-yl]-7H-imidazo[4,5-c]pyridazin-6-yl)methyl]piperidine (Compound 475a)

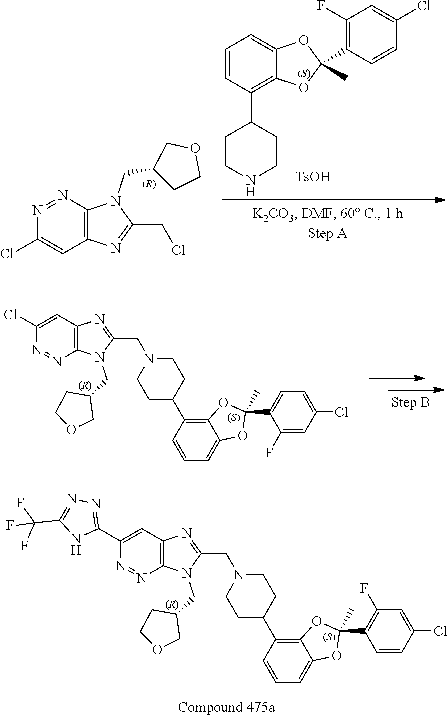

### Step A: 3-chloro-6-((4-((S)-2-(4-chloro-2-fluorophenyl)-2-methylbenzo[d][1,3]dioxol-4-yl)piperidin-1-yl)methyl)-7-(((R)-tetrahydrofuran-3-yl)methyl)-7H-imidazo[4,5-c]pyridazine

A mixture of (S)-4-(2-(4-chloro-2-fluorophenyl)-2-methylbenzo[d][1,3]dioxol-4-yl)piperidine. TsOH salt (691 mg, 1.3 mmol), (R)-3-chloro-6-(chloromethyl)-7-((tetrahydrofuran-3-yl)methyl)-7H-imidazo[4,5-c]pyridazine (380 mg, 1.3 mmol), K₂CO₃ (550 mg, 4.0 mmol) in DMF (10.0 mL) was stirred at 60° C. for 1 hour. After the reaction was completed, the mixture was diluted with EA (60 mL), washed with H₂O (30 mL*2). The combined organic layer was dried over anhydrous sodium sulfate, filtered and evaporated to dryness. The residue was purified by column chromatography on silica gel (PE/EA=1/1) to give 3-chloro-6-((4-((S)-2-(4-chloro-2-fluorophenyl)-2-methylbenzo[d][1,3]dioxol-4-yl)piperidin-1-yl)methyl)-7-(((R)-tetrahydrofuran-3-yl)methyl)-7H-imidazo[4,5-c]pyridazine (730 mg, yield: 92%) as yellow oil. MS Calcd.: 597.2; MS Found: 598.2 [M+H]⁺.

### Step B: 4-[(2S)-2-(4-chloro-2-fluorophenyl)-2-methyl-2H-1,3-benzodioxol-4-yl]-1-[(7-{[(3R)-oxolan-3-yl]methyl}-3-[5-(trifluoromethyl)-4H-1,2,4-triazol-3-yl]-7H-imidazo[4,5-c]pyridazin-6-yl)methyl]piperidine (Compound 475a)

4-[(2S)-2-(4-chloro-2-fluorophenyl)-2-methyl-2H-1,3-benzodioxol-4-yl]-1-[(7-{[(3R)-oxolan-3-yl]methyl}-3-[5-(trifluoromethyl)-4H-1,2,4-triazol-3-yl]-7H-imidazo[4,5-c]pyridazin-6-yl)methyl]piperidine (Compound 475a) (14 mg) as a white solid.

MS Calcd.: 698.2; MS Found: 699.1 [M+H]⁺.

¹H NMR (400 MHz, DMSO-d₆): δ 8.60 (s, 1H), 7.50-7.64 (m, 2H), 7.35 (dd, J=8.4 Hz, 1.6 Hz, 1H), 6.76-6.86 (m, 3H), 4.70-5.10 (m, 1H), 4.59 (d, J=7.6 Hz, 2H), 3.88-3.94 (m, 1H), 3.60-3.75 (m, 7H), 2.80-3.10 (m, 3H), 1.90-2.07 (m, 8H), 1.74-1.85 (m, 1H). ¹⁹F NMR (377 MHz, DMSO-d6): δ −67.67, −110.55.

### Example 281: 5-chloro-2-[(2S)-4-[1-({3-[(2R)-2-(difluoromethoxy)propyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]pyridine (Compound 476a)

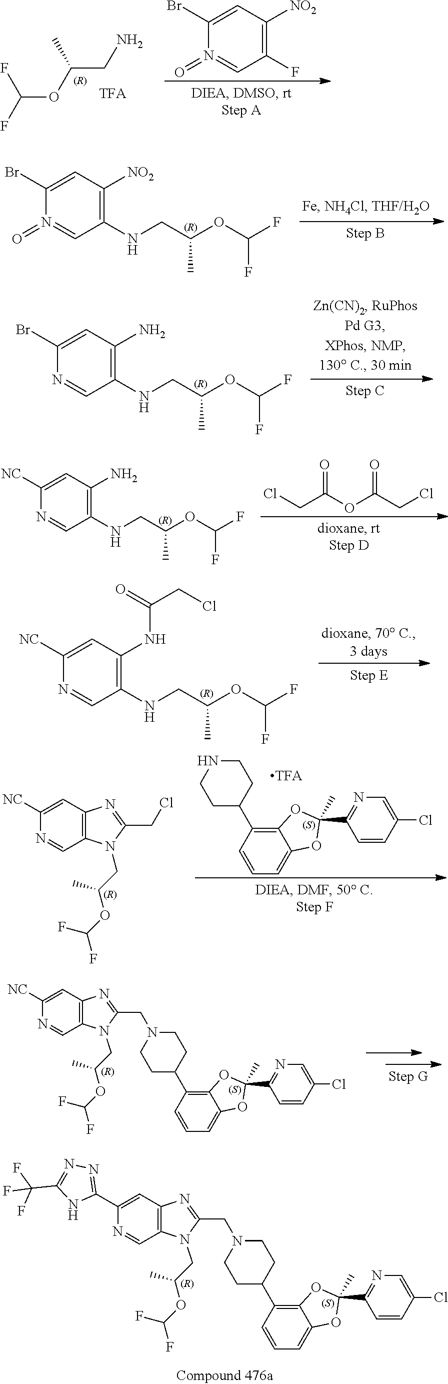

### Step A: (R)-2-bromo-5-((2-(difluoromethoxy)propyl)amino)-4-nitropyridine 1-oxide

A solution of 2-bromo-5-fluoro-4-nitropyridine 1-oxide (142 mg, 0.6 mmol), (R)-2-(difluoromethoxy) propan-1-amine TFA salt (crude, 316 mg, 1.0 mmol), DIEA (387 mg, 3.0 mmol) in DMSO (2 mL) was stirred at rt for 2 hours. The reaction was poured into water (200 mL). The precipitate was dried under vacuum to give (R)-2-bromo-5-((2-(difluoromethoxy)propyl)amino)-4-nitropyridine 1-oxide (130 mg, 63.4% yield) as a yellow solid. MS Calcd.: 341.0; MS Found: 341.9 [M+H]⁺.

### Step B: (R)-6-bromo-N3-(2-(difluoromethoxy)propyl)pyridine-3,4-diamine

To a mixture of (R)-2-bromo-5-((2-(difluoromethoxy)propyl)amino)-4-nitropyridine 1-oxide (140 mg) in THE (30 mL) and water (0.3 mL) was added ammonium chloride (85 mg, 1.6 mmol), iron powder (92 g, 1.6 mmol). The mixture was stirred at 70° C. for 5 hours. The reaction mixture was filtered through a celite pad. The filtrate was diluted with ethyl acetate (50 mL) and washed with water (50 mL). The organic layer was dried over Na₂SO₄, filtered and evaporated to dryness. The residue was purified by column chromatography on silica gel (PE/EA=2/1) to give (R)-6-bromo-N3-(2-(difluoromethoxy)propyl)pyridine-3,4-diamine (65 mg, yield: 55.1%) as yellow oil. MS Calcd.: 295.0; MS Found: 296.2 [M+H]⁺.

### Step C: (R)-4-amino-5-((2-(difluoromethoxy)propyl)amino)picolinonitrile

A mixture of (R)-6-bromo-N3-(2-(difluoromethoxy)propyl)pyridine-3,4-diamine (150 mg, 0.51 mmol), Zn(CN)₂ (89 mg, 0.76 mmol), Ruphos Pd G₃ (42 mg, 0.05 mmol) and Xphos (24 mg, 0.05 mmol) in NMP (2 mL) was stirred at 130° C. for 30 min under N₂. After the reaction was completed, the mixture was diluted with EA (60 mL), washed with H₂O (30 mL). The organic layer was dried over anhydrous sodium sulfate, filtered and evaporated to dryness. The residue was purified by Prep-TLC (PE/EA=1/1) to give (R)-4-amino-5-((2-(difluoromethoxy)propyl)amino)picolinonitrile (64 mg, yield: 53%) as a white solid. MS Calcd.: 242.1; MS Found: 243.1 [M+H]⁺.

### Step D: (R)-2-chloro-N-(2-cyano-5-((2-(difluoromethoxy)propyl)amino)pyridin-4-yl)acetamide

A mixture of (R)-4-amino-5-((2-(difluoromethoxy)propyl)amino)picolinonitrile (64 mg, 0.26 mmol) and 2-chloroacetic anhydride (90 mg, 0.53 mmol) in dioxane (2 mL) was stirred at rt overnight. The mixture was diluted with EA (60 mL), washed with sat. aqueous NaHCO₃ (20 mL). The organic layer was dried over anhydrous sodium sulfate, filtered and evaporated in vacuo to dryness. The reaction mixture was concentrated to give (R)-2-chloro-N-(2-cyano-5-((2-(difluoromethoxy)propyl)amino)pyridin-4-yl)acetamide (75 mg, crude) as a yellow solid. MS Calcd.: 318.1; MS Found: 318.9 [M+H]⁺.

### Step E: (R)-2-(chloromethyl)-3-(2-(difluoromethoxy)propyl)-3H-imidazo[4,5-c]pyridine-6-carbonitrile

A mixture of (R)-2-chloro-N-(2-cyano-5-((2-(difluoromethoxy)propyl)amino)pyridin-4-yl)acetamide (crude 75 mg), AcOH (0.4 mL) in dioxane (4 mL) was stirred at 70° C. for 3 days. The mixture was diluted with EA (40 mL), washed with Sat. NaHCO₃ (20 mL). The organic layer was dried over anhydrous sodium sulfate, filtered and evaporated in vacuo to dryness. The residue was purified by column chromatography on silica gel (EA/PE=1/1) to give (R)-2-(chloromethyl)-3-(2-(difluoromethoxy)propyl)-3H-imidazo[4,5-c]pyridine-6-carbonitrile (58 mg, yield: 74%, two steps) as yellow oil. MS Calcd.: 300.1; MS Found: 301.0 [M+H]⁺.

### Step F: 2-((4-((S)-2-(5-chloropyridin-2-yl)-2-methylbenzo[d][1,3]dioxol-4-yl)piperidin-1-yl)methyl)-3-((R)-2-(difluoromethoxy)propyl)-3H-imidazo[4,5-c]pyridine-6-carbonitrile

A mixture of (S)-5-chloro-2-(2-methyl-4-(piperidin-4-yl)benzo[d][1,3]dioxol-2-yl)pyridine. TFA salt (crude, 160 mg, 0.35 mmol), (R)-2-(chloromethyl)-3-(2-(difluoromethoxy)propyl)-3H-imidazo[4,5-c]pyridine-6-carbonitrile (58 mg, 0.19 mmol), DIEA (123 mg, 0.95 mmol) in DMF (2 mL) was stirred at 50° C. for 4 hours. After the reaction was completed, the mixture was diluted with EA (50 mL), washed with H₂O (40 mL*3). The combined organic layer was dried over anhydrous sodium sulfate, filtered and evaporated to dryness. The residue was purified by column chromatography on silica gel (DCM/MeOH=100/1) to give 2-((4-((S)-2-(5-chloropyridin-2-yl)-2-methylbenzo[d][1,3]dioxol-4-yl)piperidin-1-yl)methyl)-3-((R)-2-(difluoromethoxy)propyl)-3H-imidazo[4,5-c]pyridine-6-carbonitrile (35 mg, yield: 31.0%) as colorless oil. MS Calcd.: 594.2; MS Found: 595.1 [M+H]⁺.

### Step G: 5-chloro-2-[(2S)-4-[1-({3-[(2R)-2-(difluoromethoxy)propyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]pyridine (Compound 476a)

5-chloro-2-[(2S)-4-[1-({3-[(2R)-2-(difluoromethoxy)propyl]-6-[5-(trifluoromethyl)-4H-1,2,4-triazol-3-yl]-3H-imidazo[4,5-c]pyridin-2-yl}methyl)piperidin-4-yl]-2-methyl-2H-1,3-benzodioxol-2-yl]pyridine (Compound 476a) (10 mg) as a white solid.

MS Calcd.: 704.2; MS Found: 705.4 [M+H]⁺.

¹H NMR (400 MHz, DMSO-d₆): δ 9.08 (s, 1H), 8.70 (d, J=2.0 Hz, 1H), 8.27 (s, 1H), 7.98 (dd, J=8.4 Hz, 2.4 Hz, 1H), 7.59 (d, J=8.4 Hz, 1H), 6.70-6.82 (m, 3H), 6.46 (t, J=75.2 Hz, 1H), 4.90-5.00 (m, 1H), 4.58-4.72 (m, 2H), 4.09 (d, J=13.6 Hz, 1H), 3.75 (d, J=13.6 Hz, 1H), 3.08-3.14 (m, 1H), 2.80-2.86 (m, 1H), 2.60-2.72 (m, 1H), 2.25-2.35 (m, 1H), 2.10-2.21 (m, 1H), 2.00 (s, 3H), 1.68-1.83 (m, 4H), 1.39 (d, J=6.4 Hz, 3H). ¹⁹F NMR (377 MHz, DMSO-d₆): δ −63.62, −80.12, −80.55, −81.24, −81.67.

### Example 282: 5-chloro-2-[(2S)-2-methyl-4-(1-{[3-(5-methyl-4H-1,2,4-triazol-3-yl)-7-{[(2S)-oxetan-2-yl]methyl}-7H-imidazo[4,5-c]pyridazin-6-yl]methyl}piperidin-4-yl)-2H-1,3-benzodioxol-2-yl]pyridine (Compound 477a)

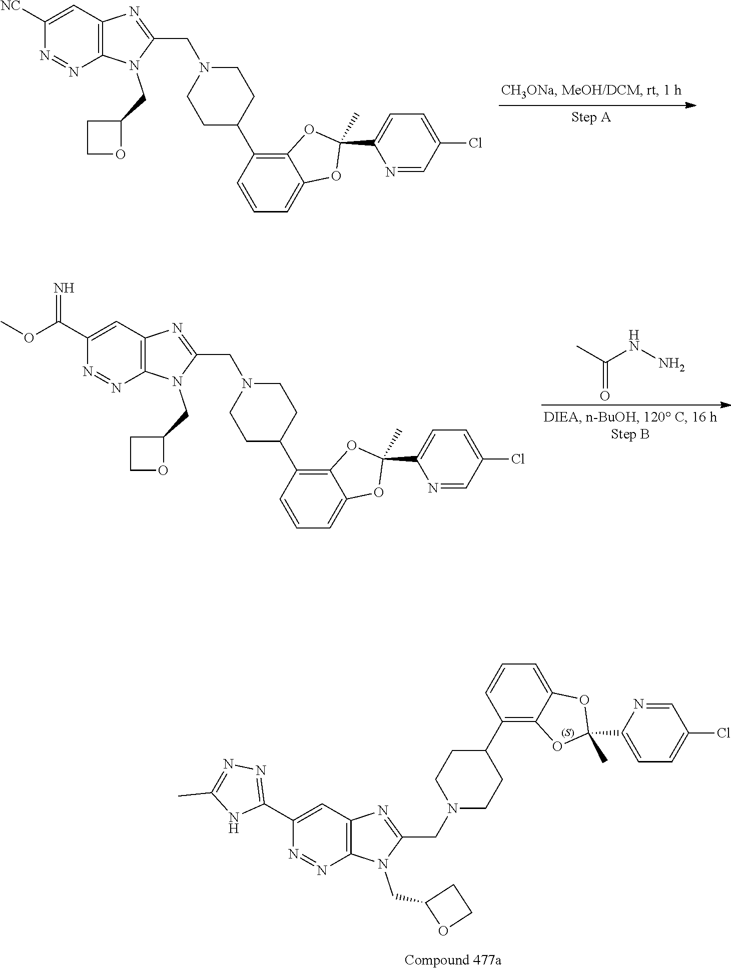

### Step A: methyl 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carbimidate

A mixture of 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carbonitrile (270 mg, 0.48 mmol), Sodium methoxide (262 mg, 4.8 mmol) in DCM/MeOH (2.0 mL/4.0 mL) was stirred at room temperature for 1 hour. After the reaction was completed, the mixture was concentrated in vacuum. The residue was purified by column chromatography on silica gel (DCM/MeOH=30/1) to give methyl 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carbimidate (330 mg, crude) as yellow oil. MS Calcd.: 589.2; MS Found: 590.2 [M+H]⁺.

### Step B: 5-chloro-2-[(2S)-2-methyl-4-(1-{[3-(5-methyl-4H-1,2,4-triazol-3-yl)-7-{[(2S)-oxetan-2-yl]methyl}-7H-imidazo[4,5-c]pyridazin-6-yl]methyl}piperidin-4-yl)-2H-1,3-benzodioxol-2-yl]pyridine (Compound 477a)

A mixture of methyl 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carbimidate (160 mg, crude mmol), acetohydrazide (40 mg, 0.54 mmol), DIEA (104 mg, 0.81 mmol) in n-BuOH (2.0 mL) was stirred at 120° C. for 16 hours. After the reaction was completed, the reaction mixture was concentrated and the residue purified by Prep-HPLC (0.1% FA/H₂O/CH₃CN) to give 5-chloro-2-[(2S)-2-methyl-4-(1-{[3-(5-methyl-4H-1,2,4-triazol-3-yl)-7-{[(2S)-oxetan-2-yl]methyl}-7H-imidazo[4,5-c]pyridazin-6-yl]methyl}piperidin-4-yl)-2H-1,3-benzodioxol-2-yl]pyridine (Compound 477a) (26 mg, yield: 18%, 2 steps) as a white solid.

MS Calcd.: 613.2; MS Found: 614.3 [M+H]⁺.

¹H NMR (400 MHz, DMSO-d₆): 8.72 (d, J=2.0 Hz, 1H), 8.30-8.41 (m, 1H), 8.01 (dd, J=8.4 Hz, 2.4 Hz, 1H), 7.60 (d, J=8.4 Hz, 1H), 6.74-6.83 (m, 3H), 5.24-5.31 (m, 1H), 4.95-5.02 (m, 1H), 4.82-4.88 (m, 1H), 4.45-4.52 (m, 1H), 4.37-4.43 (m, 1H), 3.96-4.07 (m, 2H), 2.94-3.05 (m, 2H), 2.60-2.75 (m, 2H), 2.25-2.60 (m, 6H), 2.01 (s, 3H), 1.70-1.85 (m, 4H).

### Example 283: (3,4-trans)-4-{3-[(4-chloro-2-fluorophenyl)methoxy]-4-fluorophenyl}-3-fluoro-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 478a)

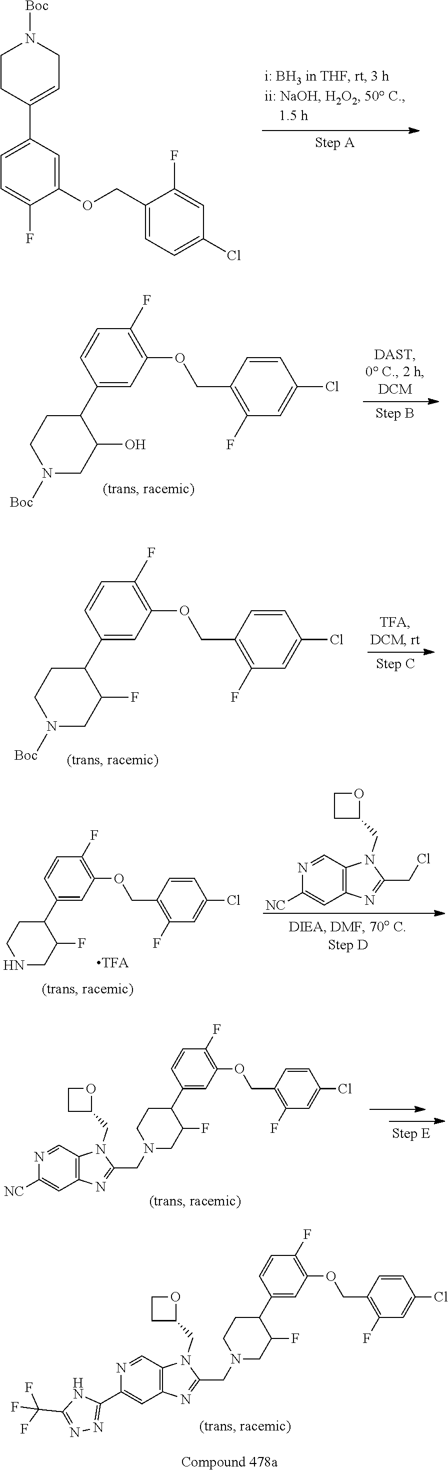

### Step A: tert-butyl (3,4-trans)-4-{3-[(4-chloro-2-fluorophenyl)methoxy]-4-fluorophenyl}-3-hydroxypiperidine-1-carboxylate

To a solution of tert-butyl 4-(3-((4-chloro-2-fluorobenzyl)oxy)-4-fluorophenyl)-3,6-dihydropyridine-1(2H)-carboxylate (1.36 g, 3.1 mmol) in THF (55 mL) was added BH₃ (6.2 mL, 6.2 mmol, 1 M in THF) at room temperature, and the mixture was stirred for 3 h. Then 2 M NaOH aqueous solution (4.7 mL, 9.3 mmol) was added, the mixture was stirred at room temperature for 10 mins, Hydrogen peroxide (3 mL, 30% aqueous solution) was added, the mixture was heated to 50° C. and stirred for 1.5 h. The resulting mixture diluted with EA (100 mL) and washed with water (50 mL*2). The organic layer was dried over Na₂SO₄, filtered and concentrated. The residue was purified by column chromatography on silica gel (PE/EA=3/1) to give tert-butyl (3,4-trans)-4-{3-[(4-chloro-2-fluorophenyl)methoxy]-4-fluorophenyl}-3-hydroxypiperidine-1-carboxylate (1.15 g, 82% yield) as colorless oil.

MS Calcd.: 453.2; MS Found: 476.0 [M+23]⁺.

¹H NMR (400 MHz, CDCl₃): δ 7.48 (t, J=8.4 Hz, 1H), 7.17 (dd, J=8.4 Hz, 2.0 Hz, 1H), 7.13 (dd, J=8.4 Hz, 2.0 Hz, 1H), 7.06 (dd, J=10.8 Hz, 8.4 Hz, 1H), 6.92 (dd, J=7.6 Hz, 2.0 Hz, 1H), 6.80-6.85 (m, 1H), 5.14 (s, 2H), 4.33-4.47 (m, 1H), 4.09-4.30 (m, 1H), 3.62 (dt, J=9.6 Hz, 4.8 Hz, 1H), 2.68-2.82 (m, 1H), 2.55-2.67 (m, 1H), 2.44-2.53 (m, 1H), 1.60-1.85 (m, 2H), 1.48 (s, 9H).

### Step B: tert-butyl (3,4-trans)-4-{3-[(4-chloro-2-fluorophenyl)methoxy]-4-fluorophenyl}-3-fluoropiperidine-1-carboxylate

To a solution of tert-butyl (3,4-trans)-4-{3-[(4-chloro-2-fluorophenyl)methoxy]-4-fluorophenyl}-3-hydroxypiperidine-1-carboxylate (200 mg, 0.44 mmol) in DCM (4 mL) was added DAST (213 mg, 1.32 mmol) at 0° C. and stirred for 2 h. The mixture was diluted with EA (50 mL) and wished with water (50 mL×2). The organic layer was dried over Na₂SO₄, filtered and evaporated to dryness. The residue was purified by column chromatography on silica gel (PE:EA=10:1) to give tert-butyl (3,4-trans)-4-{3-[(4-chloro-2-fluorophenyl)methoxy]-4-fluorophenyl}-3-fluoropiperidine-1-carboxylate (150 mg, 75% yield) as colorless oil.

MS Calcd.: 455.2; MS Found: 356.0 [M-Boc+H]⁺.

¹H NMR (400 MHz, CDCl₃): δ 7.49 (t, J=8.0 Hz, 1H), 7.17 (dd, J=8.0 Hz, 1.2 Hz, 1H), 7.13 (dd, J=9.6 Hz, 2.0 Hz, 1H), 7.06 (dd, J=10.8 Hz, 8.0 Hz, 1H), 6.91 (dd, J=7.6 Hz, 1.6 Hz, 1H), 6.80-6.85 (m, 1H), 5.15 (s, 2H), 4.43-4.58 (m, 1.5H), 4.30-4.24 (m, 0.5H), 4.05-4.25 (m, 1H), 2.68-2.79 (m, 3H), 1.82-1.93 (m, 1H), 1.60-1.75 (m, 1H), 1.49 (s, 9H).

### Step C: (3,4-trans)-4-{3-[(4-chloro-2-fluorophenyl)methoxy]-4-fluorophenyl}-3-fluoropiperidine TFA salt

A mixture of tert-butyl (3,4-trans)-4-{3-[(4-chloro-2-fluorophenyl)methoxy]-4-fluorophenyl}-3-fluoropiperidine-1-carboxylate (150 mg, 0.33 mmol), TFA (0.5 mL) in DCM (2 mL) was stirred at room temperature for 2 h. After the reaction was completed, the mixture was concentrated in vacuum to give (3,4-trans)-4-{3-[(4-chloro-2-fluorophenyl)methoxy]-4-fluorophenyl}-3-fluoropiperidine. TFA salt (crude, 201 mg) as yellow oil. MS Calcd.: 355.1; MS Found: 356.0 [M+H]⁺.

### Step D: 2-{[(3,4-trans)-4-{3-[(4-chloro-2-fluorophenyl)methoxy]-4-fluorophenyl}-3-fluoropiperidin-1-yl]methyl}-3-{[(2S)-oxetan-2-yl]methyl}-3H-imidazo[4,5-c]pyridine-6-carbonitrile

A mixture of (3,4-trans)-4-{3-[(4-chloro-2-fluorophenyl)methoxy]-4-fluorophenyl}-3-fluoropiperidine TFA salt (201 mg, crude), (S)-2-(chloromethyl)-3-(oxetan-2-ylmethyl)-3H-imidazo[4,5-c]pyridine-6-carbonitrile (90 mg, 0.33 mmol), DIEA (213 mg, 1.65 mmol) in DMF (2 mL) was stirred at 70° C. for 5 h. After the reaction was completed, the mixture was concentrated and the residue was purified by column chromatography on silica gel (DCM/MeOH=100/1) to give 2-{[(3,4-trans)-4-{3-[(4-chloro-2-fluorophenyl)methoxy]-4-fluorophenyl}-3-fluoropiperidin-1-yl]methyl}-3-{[(2S)-oxetan-2-yl]methyl}-3H-imidazo[4,5-c]pyridine-6-carbonitrile (133 mg, 69% yield) as a yellow solid. MS Calcd.: 581.2; MS Found: 582.1 [M+H]⁺.

### Step E: (3,4-trans)-4-{3-[(4-chloro-2-fluorophenyl)methoxy]-4-fluorophenyl}-3-fluoro-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 478a)

(3,4-trans)-4-{3-[(4-chloro-2-fluorophenyl)methoxy]-4-fluorophenyl}-3-fluoro-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidine (Compound 478a) (37 mg) as a white solid.

MS Calcd.: 691.2; MS Found: 692.4 [M+H]⁺.

¹H NMR (400 MHz, DMSO-d₆): δ 9.17 (d, J=2.0 Hz, 1H), 8.30 (s, 1H), 7.60 (t, J=8.0 Hz, 1H), 7.50 (dd, J=10.0 Hz, 2.0 Hz, 1H), 7.35 (dd, J=8.4 Hz, 2.0 Hz, 1H), 7.27 (d, J=8.0 Hz, 1H), 7.16 (dd, J=11.2 Hz, 8.4 Hz, 1H), 6.90-6.95 (m, 1H), 5.21 (s, 2H), 5.10-5.20 (m, 1H), 4.86-4.97 (m, 1H), 4.60-4.85 (m, 2H), 4.48-4.56 (m, 1H), 4.36-4.46 (m, 1H), 4.06-4.13 (m, 1H), 3.97-4.05 (m, 1H), 3.30-3.45 (m, 1H), 2.65-3.00 (m, 3H), 2.40-2.53 (m, 1H), 2.20-2.40 (m, 2H), 1.60-1.87 (m, 2H). ¹⁹F NMR (377 MHz, DMSO-d₆): δ −63.55 (s), −115.07 (s), −137.39 (d).

### Example 284: (2S)-4-{3-[(4-chloro-2-fluorophenyl)methoxy]-4-fluorophenyl}-2-methyl-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 479a)

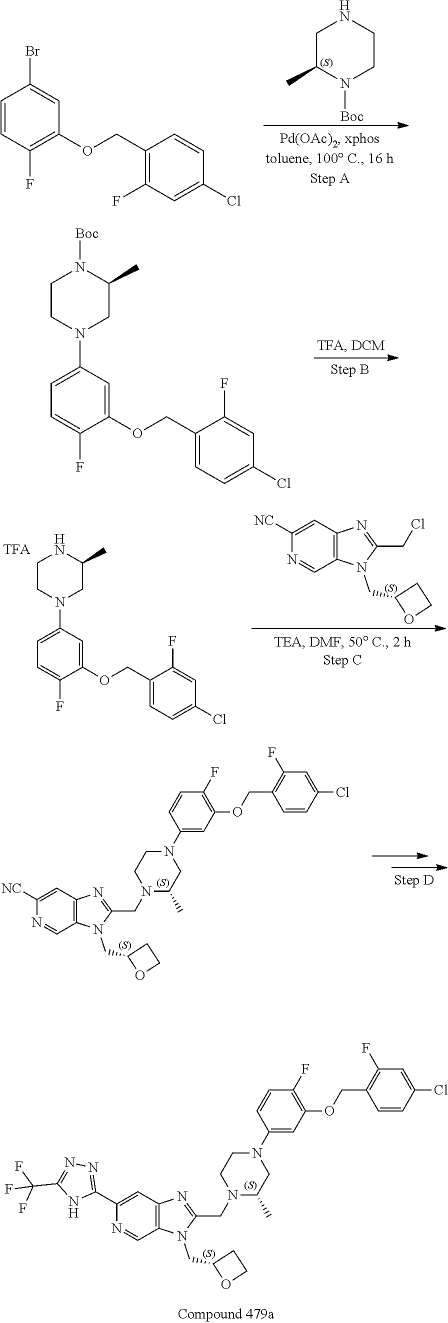

### Step A: tert-butyl (S)-4-(3-((4-chloro-2-fluorobenzyl)oxy)-4-fluorophenyl)-2-methylpiperazine-1-carboxylate

A mixture of 4-bromo-2-((4-chloro-2-fluorobenzyl)oxy)-1-fluorobenzene (3.0 g, 9.0 mmol), tert-butyl (S)-2-methylpiperazine-1-carboxylate (181 mg, 9.0 mmol), Cs₂CO₃ (8.83 g, 27.0 mmol), Pd(OAc)₂ (203 mg, 0.9 mmol) and Xphos (862 mg, 1.8 mmol) in toluene (30 mL) was stirred at 100° C. for 16 hours. After the reaction was completed, the reaction was filtered, and the filtrate concentrated in vacuum. The crude product was purified by column chromatography (PE:EA=10:1) to give tert-butyl (S)-4-(3-((4-chloro-2-fluorobenzyl)oxy)-4-fluorophenyl)-2-methylpiperazine-1-carboxylate (3.31 g, 81% yield) as yellow oil. MS Calcd.: 452.2; MS Found: 453.1 [M+H]⁺.

### Step B: (S)-1-(3-((4-chloro-2-fluorobenzyl)oxy)-4-fluorophenyl)-3-methylpiperazine TFA salt

A mixture of tert-butyl (S)-4-(3-((4-chloro-2-fluorobenzyl)oxy)-4-fluorophenyl)-2-methylpiperazine-1-carboxylate (173 mg, 0.38 mmol) in DCM (2 mL) was added TFA (1 mL). The reaction was stirred at room temperature for 1 hour. After the reaction was completed, the reaction was concentrated in vacuum to give (S)-1-(3-((4-chloro-2-fluorobenzyl)oxy)-4-fluorophenyl)-3-methylpiperazine TFA salt (450 mg, crude) as yellow oil. MS Calcd: 352.1; MS Found: 353.3 [M+H]⁺.

### Step C: 2-(((S)-4-(3-((4-chloro-2-fluorobenzyl)oxy)-4-fluorophenyl)-2-methylpiperazin-1-yl)methyl)-3-(((S)-oxetan-2-yl)methyl)-3H-imidazo[4,5-c]pyridine-6-carbonitrile

A mixture of (S)-1-(3-((4-chloro-2-fluorobenzyl)oxy)-4-fluorophenyl)-3-methylpiperazine TFA salt (450 mg, crude), (S)-2-(chloromethyl)-3-(oxetan-2-ylmethyl)-3H-imidazo[4,5-c]pyridine-6-carbonitrile (80 mg, 0.30 mmol) and TEA (1 mL) in DMF (2 mL) was stirred at 50° C. for 2 hours. After the reaction was completed, the reaction was quenched with H₂O (20 mL) and extracted with ethyl acetate (25 mL×3). The organic layer was combined and washed with brine (20 mL×2), dried over sodium sulfate, filtered and concentrated in vacuum. The reaction was purified by column chromatography (DCM:MeOH=100:1) to give 2-(((S)-4-(3-((4-chloro-2-fluorobenzyl)oxy)-4-fluorophenyl)-2-methylpiperazin-1-yl)methyl)-3-(((S)-oxetan-2-yl)methyl)-3H-imidazo[4,5-c]pyridine-6-carbonitrile (122 mg, 55% yield, two steps) as yellow oil. MS Calcd.: 578.2; MS Found: 579.2 [M+H]⁺.

### Step D: (2S)-4-{3-[(4-chloro-2-fluorophenyl)methoxy]-4-fluorophenyl}-2-methyl-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 479a)

(2S)-4-{3-[(4-chloro-2-fluorophenyl)methoxy]-4-fluorophenyl}-2-methyl-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 479a) (6.9 mg) as a white solid.

MS Calcd.: 688.2; MS Found: 689.2 [M+H]⁺.

¹H NMR (400 MHz, DMSO-d₆): 9.16 (s, 1H), 8.29 (s, 1H), 7.59 (t, J=8.0 Hz, 1H), 7.50 (dd, J=10.0 Hz, J=2.0 Hz, 1H), 7.35 (dd, J=8.4 Hz, J=1.6 Hz, 1H), 7.03 (dd, J=10.8 Hz, J=8.8 Hz, 1H), 6.80-6.86 (m, 1H), 6.43-6.50 (m, 1H), 5.20-5.25 (m, 1H), 5.18 (s, 2H), 4.87 (d, J=4.4 Hz, 2H), 4.47-4.53 (m, 1H), 4.43 (d, J=14.4 Hz, 1H), 4.30-4.38 (m, 1H), 3.74 (d, J=14.4 Hz, 1H), 3.40-3.48 (m, 1H), 2.65-2.81 (m, 4H), 2.55-2.62 (m, 1H), 2.37-2.52 (m, 3H), 1.17 (d, J=6.0 Hz, 3H). ¹⁹F NMR (377 MHz, DMSO-d₆): δ −63.57, −115.00, −146.15.

### Example 285: (2S)-4-{6-[(4-chloro-2-fluorophenyl)methoxy]pyridin-2-yl}-2-methyl-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 480a)

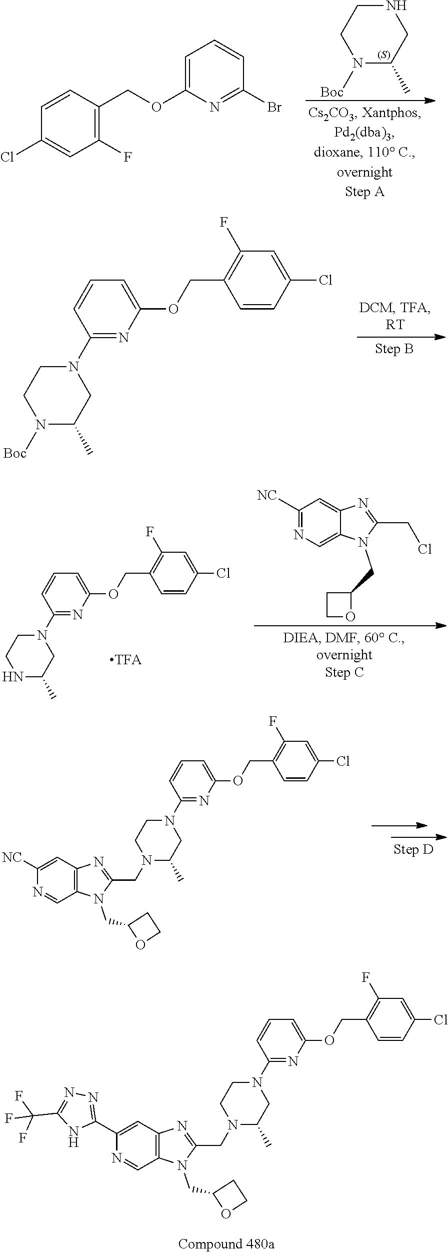

### Step A: tert-butyl (S)-4-(6-((4-chloro-2-fluorobenzyl)oxy)pyridin-2-yl)-2-methylpiperazine-1-carboxylate

A mixture of 2-bromo-6-((4-chloro-2-fluorobenzyl)oxy)pyridine (310 mg, 0.98 mmol), tert-butyl (S)-2-methylpiperazine-1-carboxylate (216 mg, 1.08 mmol), Cs₂CO₃ (799 mg, 2.5 mmol), Xantphos (114 mg, 0.20 mmol), Pd₂(dba)₃ (90 mg, 0.098 mmol) in dioxane (5 mL) was stirred at 110° C. overnight. Upon completion, the mixture was diluted with ethyl acetate (50 mL), washed with brine (30 mL×2). The organic layer was dried over sodium sulfate, filtered and concentrated in vacuum. The residue was purified by column chromatography on silica gel (PE/EA=6/1) to give tert-butyl (S)-4-(6-((4-chloro-2-fluorobenzyl)oxy)pyridin-2-yl)-2-methylpiperazine-1-carboxylate (285 mg, yield: 67%) as yellow oil. MS Calcd.: 435.2; MS Found: 436.4 [M+H]⁺.

### Step B: (S)-1-(6-((4-chloro-2-fluorobenzyl)oxy)pyridin-2-yl)-3-methylpiperazine TFA salt

A mixture of tert-butyl (S)-4-(6-((4-chloro-2-fluorobenzyl)oxy)pyridin-2-yl)-2-methylpiperazine-1-carboxylate (103 mg, 1.01 mmol) and TFA (150 mg, 3.03 mmol) in DCM (3 mL) was stirred at room temperature for 1 hour. Upon completion, the mixture was concentrated in vacuum to give (S)-1-(6-((4-chloro-2-fluorobenzyl)oxy)pyridin-2-yl)-3-methylpiperazine TFA salt (200 mg, crude) as yellow oil. MS Calcd.: 335.1; MS Found: 336.1 [M+H]⁺.

### Step C: 2-(((S)-4-(6-((4-chloro-2-fluorobenzyl)oxy)pyridin-2-yl)-2-methylpiperazin-1-yl)methyl)-3-(((S)-oxetan-2-yl)methyl)-3H-imidazo[4,5-c]pyridine-6-carbonitrile

A mixture of (S)-1-(6-((4-chloro-2-fluorobenzyl)oxy)pyridin-2-yl)-3-methylpiperazine TFA salt (crude, 200 mg), (S)-2-(chloromethyl)-3-(oxetan-2-ylmethyl)-3H-imidazo[4,5-c]pyridine-6-carbonitrile (80 mg, 0.7 mmol), DIEA (1.3 g, 10.1 mmol) in DMF (3 mL) was stirred at 60° C. overnight. After cooling down to room temperature, the mixture was diluted with ethyl acetate (50 mL), washed with brine (50 mL×2). The organic layer was dried over sodium sulfate, filtered and concentrated in vacuum. The residue was purified by column chromatography on silica gel (PE/EA=1/2) to give 2-(((S)-4-(6-((4-chloro-2-fluorobenzyl)oxy)pyridin-2-yl)-2-methylpiperazin-1-yl)methyl)-3-(((S)-oxetan-2-yl)methyl)-3H-imidazo[4,5-c]pyridine-6-carbonitrile (120 mg, yield: 30%, two steps) as yellow oil. MS Calcd.: 561.2; MS Found: 562.3 [M+H]⁺.

### Step D: (2S)-4-{6-[(4-chloro-2-fluorophenyl)methoxy]pyridin-2-yl}-2-methyl-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 480a)

(2S)-4-{6-[(4-chloro-2-fluorophenyl)methoxy]pyridin-2-yl}-2-methyl-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 480a) (29 mg) was obtained as a white solid.

MS Calcd.: 671.2; MS Found: 672.4 [M+H]⁺.

¹H NMR (400 MHz, DMSO-d₆): δ 9.16 (s, 1H), 8.28 (s, 1H), 7.41-7.53 (m, 3H), 7.28 (dd, J=8.4 Hz, 2.0 Hz, 1H), 6.33 (d, J=8.4 Hz, 1H), 6.08 (d, J=8.0, 1H), 5.30 (s, 2H), 5.17-5.26 (m, 1H), 4.81-4.89 (m, 2H), 4.47-4.55 (m, 1H), 4.30-4.42 (m, 2H), 3.82-3.90 (m, 1H), 3.70-3.80 (m, 2H), 3.00-3.09 (m, 1H), 2.82-2.91 (m, 1H), 2.59-2.79 (m, 3H), 2.31-2.53 (m, 2H), 1.13 (d, J=6.0 Hz, 3H). ¹⁹F NMR (377 MHz, DMSO-d₆): δ −63.44, −115.47.

### Example 286: 5-[2-({4-[(2S)-2-(5-chloropyridin-2-yl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4H-1,2,4-triazole-3-carbonitrile (Compound 481a)

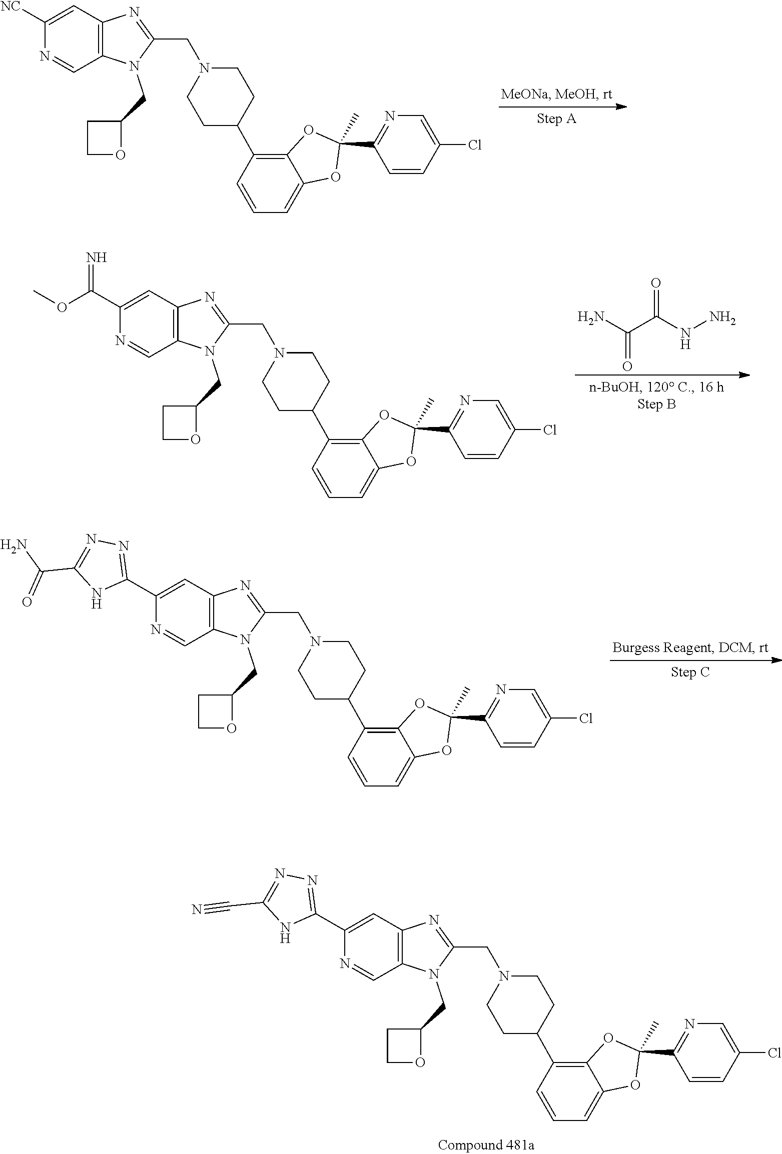

### Step A: methyl 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate

A solution of 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbonitrile (200 mg, 0.36 mmol) and MeONa (194 mg, 3.6 mmol) in MeOH (2 mL) was stirred at rt for 2 h. After the reaction was completed, the mixture was concentrated and the residue was purified by column chromatography on silica gel (DCM/MeOH=10/1) to give methyl 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (150 mg, 72% yield) as a white solid. MS Calcd.: 588.2; MS Found: 589.3[M+H]⁺.

### Step B: 5-(2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-4H-1,2,4-triazole-3-carboxamide

A mixture of methyl 2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridine-6-carbimidate (150 mg, 0.26 mmol), 2-hydrazineyl-2-oxoacetamide (53 mg, 0.51 mmol), DIEA (99 mg, 0.77 mmol) in n-BuOH (3 mL) was stirred at 120° C. for 16 hours. After the reaction was completed, the mixture was diluted with EA (20 mL), washed with H₂O (10 mL×2). The organic layer was dried over anhydrous sodium sulfate, filtered and evaporated to dryness. The residue was purified by prep-TLC (DCM/MeOH=12/1) to give 5-(2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-4H-1,2,4-triazole-3-carboxamide (120 mg, 73% yield) as a white solid. MS Calcd.: 641.2; MS Found: 642.3[M+H]⁺.

### Step C: 5-[2-({4-[(2S)-2-(5-chloropyridin-2-yl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4H-1,2,4-triazole-3-carbonitrile (Compound 481a)

A mixture of 5-(2-((4-((S)-2-(5-chloropyridin-2-yl)-2-methylbenzo[d][1,3]dioxol-4-yl)piperidin-1-yl)methyl)-3-(((S)-oxetan-2-yl)methyl)-3H-imidazo[4,5-c]pyridin-6-yl)-4H-1,2,4-triazole-3-carboxamide (120 mg, 0.187 mmol), Burgess reagent (50 mg, 0.210 mmol) in DCM (5 mL) was stirred at rt for 0.5 hours. After the reaction was completed, the reaction mixture was concentrated and the residue purified by Prep-HPLC (0.1% FA/H₂O/CH₃CN) to give 5-[2-({4-[(2S)-2-(5-chloropyridin-2-yl)-2-methyl-2H-1,3-benzodioxol-4-yl]piperidin-1-yl}methyl)-3-{[(2S)-oxetan-2-yl]methyl}-3H-imidazo[4,5-c]pyridin-6-yl]-4H-1,2,4-triazole-3-carbonitrile (Compound 481a) (7.5 mg, 6.4% yield) as a white solid.

MS Calcd.: 623.2; MS Found: 624.1[M+H]⁺.

¹H NMR (400 MHz, DMSO-d₆): δ 9.13 (s, 1H), 8.72 (d, J=2.4 Hz, 1H), 8.27 (s, 1H), 8.00 (dd, J=1.6 Hz, 8.4 Hz, 1H), 7.60 (d, J=8.4 Hz, 1H), 6.72-6.82 (m, 3H), 5.10-5.21 (m, 1H), 4.83-4.93 (m, 1H), 4.67-4.75 (m, 1H), 4.35-4.52 (m, 2H), 3.99 (d, J=13.6 Hz, 1H), 3.84 (d, J=14.0 Hz, 1H), 2.99-3.06 (m, 1H), 2.82-2.90 (m, 1H), 2.55-2.78 (m, 2H), 2.40-2.52 (m, 1H), 2.15-2.35 (m, 2H), 2.01 (s, 3H), 1.68-1.80 (m, 4H).

### Example 287: 5-[6-({4-[(2S)-2-(5-chloropyridin-2-yl)-2-methyl-2H-1,3-benzodioxol-4-yl]piperidin-1-yl}methyl)-7-{[(2S)-oxetan-2-yl]methyl}-7H-imidazo[4,5-c]pyridazin-3-yl]-4H-1,2,4-triazole-3-carbonitrile (Compound 482a)

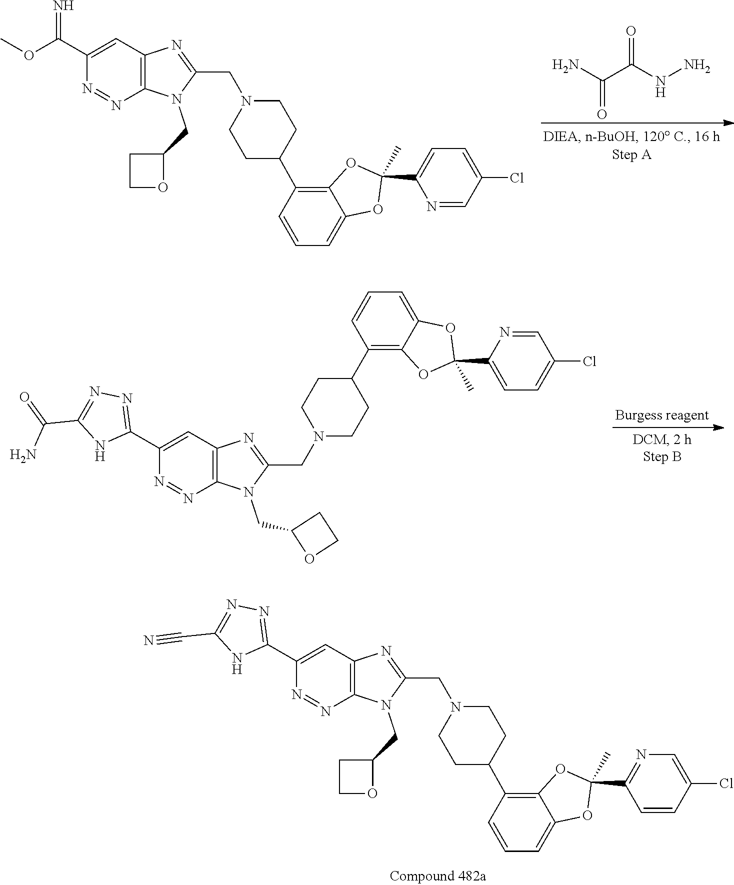

### Step A: 5-(6-((4-((R)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazin-3-yl)-4H-1,2,4-triazole-3-carboxamide

A mixture of methyl 6-((4-((S)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazine-3-carbimidate (170 mg, 0.29 mmol), 2-hydrazineyl-2-oxoacetamide (60 mg, 0.58 mmol), DIEA (112 mg, 0.87 mmol) in n-BuOH (2.0 mL) was stirred at 120° C. for 16 hours. After the reaction was completed, the mixture was diluted with EA (20.0 mL), washed with H₂O (10 mL*2). The organic layer was dried over anhydrous sodium sulfate, filtered and evaporated to dryness. The residue was purified by prep-TLC (DCM/MeOH=20/1) to give 5-(6-((4-((R)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazin-3-yl)-4H-1,2,4-triazole-3-carboxamide (120 mg, yield: 65%) as brown oil. MS Calcd.: 642.2; MS Found: 643.2 [M+H]⁺.

### Step B: 5-[6-({4-[(2S)-2-(5-chloropyridin-2-yl)-2-methyl-2H-1,3-benzodioxol-4-yl]piperidin-1-yl}methyl)-7-{[(2S)-oxetan-2-yl]methyl}-7H-imidazo[4,5-c]pyridazin-3-yl]-4H-1,2,4-triazole-3-carbonitrile (Compound 482a)

A mixture of 5-(6-((4-((R)-2-(5-chloropyridin-2-yl)-2-methylbenzo[d][1,3]dioxol-4-yl)piperidin-1-yl)methyl)-7-(((S)-oxetan-2-yl)methyl)-7H-imidazo[4,5-c]pyridazin-3-yl)-4H-1,2,4-triazole-3-carboxamide (120 mg, 0.19 mmol), Burgess reagent (177.94 mg, 0.75 mmol) in DCM (3.0 mL) was stirred at rt for 2 hours. After the reaction was completed, the mixture was diluted with EA (20 mL), washed with H₂O (10 mL×2). The organic layer was dried over anhydrous sodium sulfate, filtered and evaporated to dryness. The residue was purified by Prep-HPLC (0.1% NH3-H₂O/H₂O/CH₃CN) to give 5-[6-({4-[(2S)-2-(5-chloropyridin-2-yl)-2-methyl-2H-1,3-benzodioxol-4-yl]piperidin-1-yl}methyl)-7-{[(2S)-oxetan-2-yl]methyl}-7H-imidazo[4,5-c]pyridazin-3-yl]-4H-1,2,4-triazole-3-carbonitrile (Compound 482a) (7.9 mg, yield: 6.8%) as a white solid.

MS Calcd.: 624.2; MS Found: 625.2 [M+H]⁺.

¹H NMR (400 MHz, DMSO-d₆): 8.72 (d, J=2.4 Hz, 1H), 8.53 (s, 1H), 8.01 (dd, J=8.4 Hz, 2.4 Hz, 1H), 7.61 (d, J=8.4 Hz, 1H), 6.75-6.85 (m, 3H), 5.25-5.33 (m, 1H), 4.95-5.05 (m, 1H), 4.83-4.92 (m, 1H), 4.45-4.52 (m, 1H), 4.38-4.45 (m, 1H), 4.03-4.13 (m, 2H), 2.98-3.10 (m, 2H), 2.50-2.80 (m, 3H), 2.26-2.42 (m, 2H), 2.01 (s, 3H), 1.72-1.85 (m, 4H).

### Example 288: 1-{3-[(4-chloro-2-fluorophenyl)methoxy]-4-fluorophenyl}-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 483a)

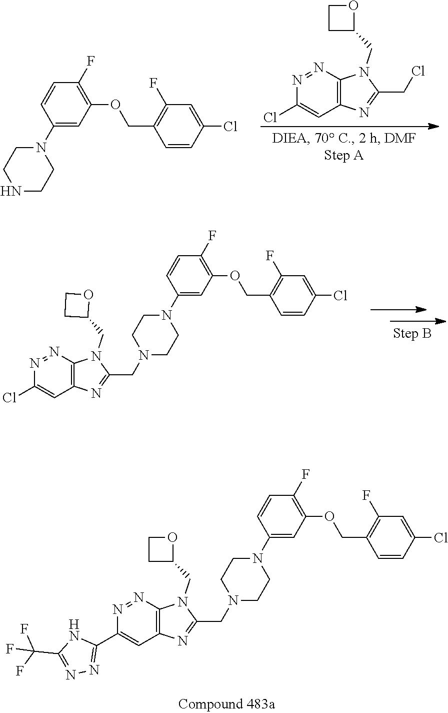 [SMILES: Fc1cc(Cl)ccc1COc1cc(N2CCNCC2)ccc1F ; ClCc1nc2cc(Cl)nnc2n1C[C@@H]1CCO1 ; Fc1cc(Cl)ccc1COc1cc(N2CCN(Cc3nc4cc(Cl)nnc4n3C[C@@H]3CCO3)CC2)ccc1F ; Fc1cc(Cl)ccc1COc1cc(N2CCN(Cc3nc4cc(-c5nnc(C(F)(F)F)[nH]5)nnc4n3C[C@@H]3CCO3)CC2)ccc1F]

### Step A: (S)-3-chloro-6-((4-(3-((4-chloro-2-fluorobenzyl)oxy)-4-fluorophenyl)piperazin-1-yl)methyl)-7-(oxetan-2-ylmethyl)-7H-imidazo[4,5-c]pyridazine

To a solution of 1-(3-((4-chloro-2-fluorobenzyl)oxy)-4-fluorophenyl)piperazine TFA Salt (410 mg, 1.2 mmol) in DMF (10 mL) was added DIEA (1.4 g, 11.0 mmol) and (S)-3-chloro-6-(chloromethyl)-7-(oxetan-2-ylmethyl)-7H-imidazo[4,5-c]pyridazine (300 mg, 1.103 mmol). The solution was stirred at 70° C. for 2 hours. The reaction was quenched with H₂O (50 mL), extracted with ethyl acetate (70 mL×2). The organic layer was dried over anhydrous sodium sulfate, filtered and evaporated to dryness. The residue was purified by column chromatography on silica gel (PE/EA=1/2) to give (S)-3-chloro-6-((4-(3-((4-chloro-2-fluorobenzyl)oxy)-4-fluorophenyl)piperazin-1-yl)methyl)-7-(oxetan-2-ylmethyl)-7H-imidazo[4,5-c]pyridazine (469 mg, 74% yield) as a red solid. MS Calcd.: 574.2; MS Found: 575.1 [M+H]⁺.

### Step B: 1-{3-[(4-chloro-2-fluorophenyl)methoxy]-4-fluorophenyl}-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 483a)

1-{3-[(4-chloro-2-fluorophenyl)methoxy]-4-fluorophenyl}-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 483a) (27 mg) was obtained as a white solid.

MS Calcd.: 675.2; MS Found: 674.1 [M−H]⁻.

¹H NMR (400 MHz, DMSO-d6): δ 8.53 (s, 1H), 7.59 (t, J=8.0 Hz, 1H), 7.50 (dd, J=10.0 Hz, 1.6 Hz, 1H), 7.35 (dd, J=8.0 Hz, 1.6 Hz, 1H), 7.00-7.08 (m, 1H), 6.85 (dd, J=7.2 Hz, 2.4 Hz, 1H), 6.45-6.52 (m, 1H), 5.23-5.31 (m, 1H), 5.18 (s, 2H), 4.99-5.07 (m, 1H), 4.85-4.93 (m, 1H), 4.48-4.56 (m, 1H), 4.39-4.46 (m, 1H), 4.05-4.14 (m, 2H), 3.10-3.20 (m, 4H), 2.66-2.80 (m, 5H), 2.50-2.60 (m, 1H). ¹⁹F NMR (377 MHz, DMSO-d₆): δ −63.66, −115.00, −146.02.

### Example 289: 5-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-c]pyridin-6-yl}-4H-1,2,4-triazole-3-carbonitrile (Compound 484a)

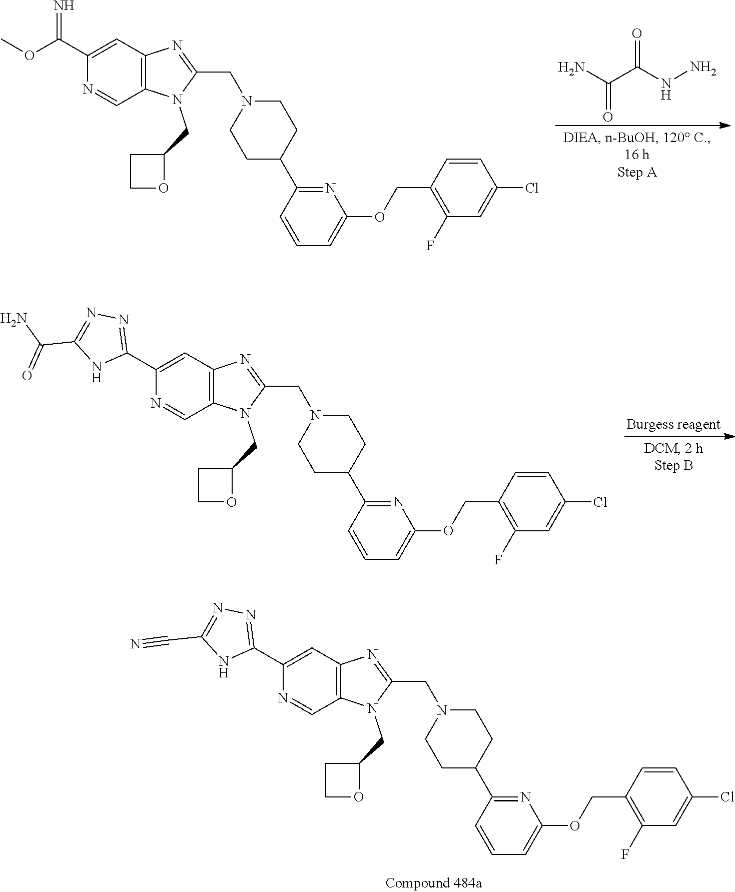 [SMILES: COC(=N)c1cc2nc(CN3CCC(c4cccc(OCc5ccc(Cl)cc5F)n4)CC3)n(C[C@@H]3CCO3)c2cn1 ; NNC(=O)C(N)=O ; NC(=O)c1nnc(-c2cc3nc(CN4CCC(c5cccc(OCc6ccc(Cl)cc6F)n5)CC4)n(C[C@@H]4CCO4)c3cn2)[nH]1 ; N#Cc1nnc(-c2cc3nc(CN4CCC(c5cccc(OCc6ccc(Cl)cc6F)n5)CC4)n(C[C@@H]4CCO4)c3cn2)[nH]1]

### Step A: (S)-5-(2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridin-6-yl)-4H-1,2,4-triazole-3-carboxamide

To a solution of methyl (S)-2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridine-6-carbimidate (220 mg, 0.38 mmol) and 2-hydrazineyl-2-oxoacetamide (78 mg, 0.76 mmol) in n-BuOH (3 mL) was added DIEA (147 mg, 1.1 mmol) and 2-hydrazineyl-2-oxoacetamide (157 mg, 1.5 mmol) at room temperature. The reaction was stirred at 120° C. for 16 hours. After the reaction was completed, the reaction was concentrated, diluted with water (5 mL) and extracted with ethyl acetate (10 mL×3). The organic layer was combined and washed with brine (5 mL), then dried over sodium sulfate, filtered and concentrated in vacuum. The residue was purified by column chromatography on silica gel (MeOH/DCM=10/1) to give (S)-5-(2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridin-6-yl)-4H-1,2,4-triazole-3-carboxamide (137 mg, yield: 57%) as a white solid. MS Calcd.: 631.2; MS Found: 632.4 [M+H]⁺.

### Step B: 5-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-c]pyridin-6-yl}-4H-1,2,4-triazole-3-carbonitrile (Compound 484a)

To a mixture of (S)-5-(2-((4-(6-((4-chloro-2-fluorobenzyl)oxy)pyridin-2-yl)piperidin-1-yl)methyl)-3-(oxetan-2-ylmethyl)-3H-imidazo[4,5-c]pyridin-6-yl)-4H-1,2,4-triazole-3-carboxamide (137 mg, 0.22 mmol), Burgess reagent (207 mg, 0.87 mmol) in DCM (2.0 mL) was stirred at rt for 2 hours. After the reaction was completed, the mixture was diluted with EA (20 mL), washed with H₂O (10 mL×2). The organic layer was dried over anhydrous sodium sulfate, filtered and evaporated to dryness. The organic layer was concentrated and the residue purified by Prep-HPLC (0.1% FA/H₂O/CH₃CN) to give 5-{2-[(4-{6-[(4-chloro-2-fluorophenyl)methoxy]pyridin-2-yl}piperidin-1-yl)methyl]-3-{[(2S)-oxetan-2-yl]methyl}-3H-imidazo[4,5-c]pyridin-6-yl}-4H-1,2,4-triazole-3-carbonitrile (Compound 484a) (45 mg, yield: 34%) as a white solid.

MS Calcd.: 613.2; MS Found: 614.1 [M+H]⁺.

¹H NMR (400 MHz, DMSO-d₆): δ 9.17 (s, 1H), 8.30 (s, 1H), 7.63 (t, J=8.0 Hz, 1H), 7.56 (t, J=8.0 Hz, 1H), 7.46 (dd, J=10.4 Hz, 2.0 Hz, 1H), 7.30 (dd, J=8.4 Hz, 2.0 Hz, 1H), 6.88 (d, J=7.2 Hz, 1H), 6.68 (d, J=8.0 Hz, 1H), 5.38 (s, 2H), 5.14-5.24 (m, 1H), 4.89-4.98 (m, 1H), 4.73-4.82 (m, 1H), 4.38-4.54 (m, 2H), 4.02 (d, J=13.6 Hz, 1H), 3.87 (d, J=13.6 Hz, 1H), 2.98-3.07 (m, 1H), 2.83-2.93 (m, 1H), 2.69-2.80 (m, 1H), 2.56-2.67 (m, 1H), 2.35-2.53 (m, 1H), 2.17-2.35 (m, 2H), 1.63-1.87 (m, 4H). ¹⁹F NMR (377 MHz, DMSO-d₆): δ −115.28.

### Example 290: 3-fluoro-4-{[(3-fluoro-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridin-2-yl)oxy]methyl}benzonitrile (Compound 485a)

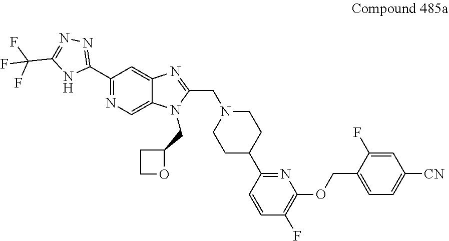 [SMILES: N#Cc1ccc(COc2nc(C3CCN(Cc4nc5cc(-c6nnc(C(F)(F)F)[nH]6)ncc5n4C[C@@H]4CCO4)CC3)ccc2F)c(F)c1]

3-fluoro-4-{[(3-fluoro-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridin-2-yl)oxy]methyl}benzonitrile (Compound 485a) (13 mg) was obtained as a white solid.

MS Calcd.: 665.2; MS Found: 666.1 [M+H]⁺.

¹H NMR (400 MHz, DMSO-d6): δ 9.15 (s, 1H), 8.27 (s, 1H), 7.90 (d, J=10.0 Hz, 1H), 7.70-7.75 (m, 2H), 7.57-7.66 (m, 1H), 6.92 (dd, J=8.4 Hz, 2.8 Hz, 1H), 5.56 (s, 2H), 5.13-5.23 (m, 1H), 4.88-4.96 (m, 1H), 4.73-4.80 (m, 1H), 4.39-4.55 (m, 2H), 3.99 (d, J=13.6 Hz, 1H), 3.85 (d, J=13.6 Hz, 1H), 2.95-3.03 (m, 1H), 2.82-2.90 (m, 1H), 2.65-2.80 (m, 1H), 2.42-2.60 (m, 2H), 2.15-2.34 (m, 2H), 1.56-1.83 (m, 4H). ¹⁹F NMR (377 MHz, DMSO-d₆): δ −63.42, −115.37, −143.99.

### Example 291: 2-[(4-chloro-2-fluorophenyl)methoxy]-6-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyridine (Compound 486a)

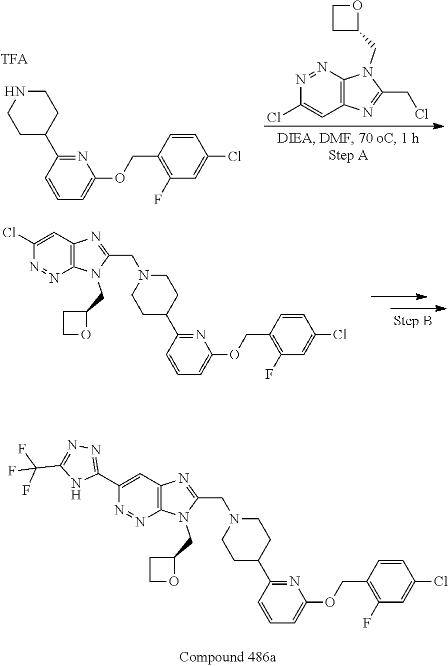

### Step A: (S)-3-chloro-6-((4-(6-((4-chloro-2-fluorobenzyl)oxy)pyridin-2-yl)piperidin-1-yl)methyl)-7-(oxetan-2-ylmethyl)-7H-imidazo[4,5-c]pyridazine

A mixture of 2-((4-chloro-2-fluorobenzyl)oxy)-6-(piperidin-4-yl)pyridine TFA salt (1.2 g, 3.74 mmol), (S)-3-chloro-6-(chloromethyl)-7-(oxetan-2-ylmethyl)-7H-imidazo[4,5-c]pyridazine (450 mg, 1.6 mmol), DIEA (2.1 g, 16.5 mmol) in DMF (8 mL) was stirred at 70° C. for 1 hour. After the reaction was completed, the mixture was diluted with EA (50 mL), washed with H₂O (30 mL*2). The combined organic layer was dried over anhydrous sodium sulfate, filtered and evaporated to dryness. The residue was purified by column chromatography on silica gel (DCM/MeOH=60/1) to give (S)-3-chloro-6-((4-(6-((4-chloro-2-fluorobenzyl)oxy)pyridin-2-yl)piperidin-1-yl)methyl)-7-(oxetan-2-ylmethyl)-7H-imidazo[4,5-c]pyridazine (630 mg, yield: 69%) as a brown solid. MS Calcd.: 556.2; MS Found: 556.9 [M+H]⁺.

### Step B: 2-[(4-chloro-2-fluorophenyl)methoxy]-6-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyridine (Compound 486a)

-[(4-chloro-2-fluorophenyl)methoxy]-6-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyridine (Compound 486a) (33 mg) was obtained as a white solid.

MS Calcd.: 657.2; MS Found: 658.0 [M+H]⁺.

¹H NMR (400 MHz, DMSO-d6): δ 8.49 (s, 1H), 7.64 (t, J=8.0 Hz, 1H), 7.57 (t, J=8.0 Hz, 1H), 7.46 (dd, J=10.0 Hz, 2.0 Hz, 1H), 7.30 (dd, J=8.4 Hz, 1.6 Hz, 1H), 6.88 (d, J=7.2 Hz, 1H), 6.68 (d, J=8.0 Hz, 1H), 5.38 (s, 2H), 5.25-5.34 (m, 1H), 5.00-5.08 (m, 1H), 4.87-4.96 (m, 1H), 4.48-4.55 (m, 1H), 4.38-4.47 (m, 1H), 3.99-4.10 (m, 2H), 2.94-3.05 (m, 2H), 2.70-2.81 (m, 1H), 2.50-2.68 (m, 2H), 2.24-2.36 (m, 2H), 1.68-1.87 (m, 4H). ¹⁹F NMR (377 MHz, DMSO-d₆): δ −63.15, −115.21.

### Example 292: 3-fluoro-4-{[(6-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyridin-2-yl)oxy]methyl}benzonitrile (Compound 487a)

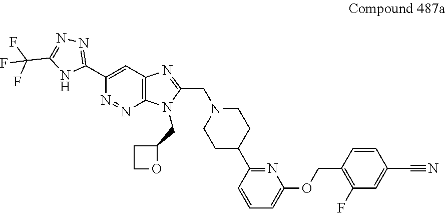 [SMILES: N#Cc1ccc(COc2cccc(C3CCN(Cc4nc5cc(-c6nnc(C(F)(F)F)[nH]6)nnc5n4C[C@@H]4CCO4)CC3)n2)c(F)c1]

3-fluoro-4-{[(6-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyridin-2-yl)oxy]methyl}benzonitrile (Compound 487a) (13 mg) was obtained as a white solid.

MS Calcd.: 648.2; MS Found: 649.1 [M+H]⁺.

¹H NMR (400 MHz, DMSO-d₆): δ 8.52 (s, 1H), 7.88 (d, J=10.4 Hz, 1H), 7.69-7.73 (m, 2H), 7.66 (t, J=7.6 Hz, 1H), 6.90 (d, J=7.2 Hz, 1H), 6.73 (d, J=8.0 Hz, 1H), 5.48 (s, 2H), 5.25-5.35 (m, 1H), 5.01-5.10 (m, 1H), 4.88-4.96 (m, 1H), 4.48-4.56 (m, 1H), 4.39-4.47 (m, 1H), 4.00-4.11 (m, 2H), 2.92-3.05 (m, 2H), 2.70-2.82 (m, 1H), 2.50-2.70 (m, 2H), 2.24-2.35 (m, 2H), 1.65-1.85 (m, 4H). ¹⁹F NMR (377 MHz, DMSO-d₆): δ −63.67, −115.50.

### Example 293: 2-[(4-chloro-2-fluorophenyl)methoxy]-3-fluoro-6-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyridine (Compound 488a)

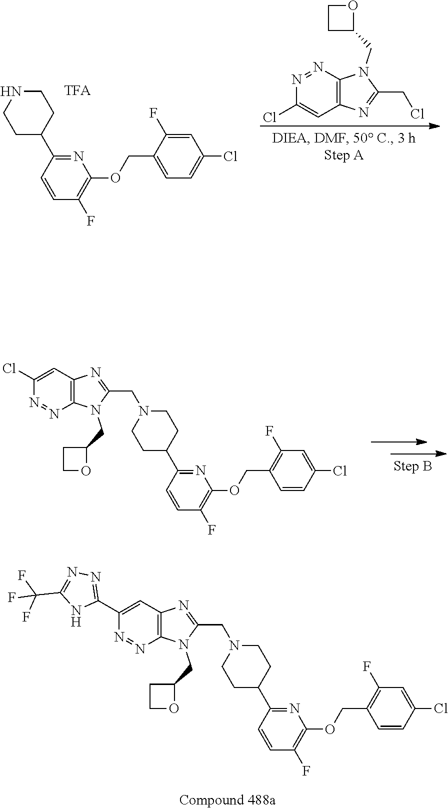 [SMILES: Fc1cc(Cl)ccc1COc1nc(C2CCNCC2)ccc1F ; ClCc1nc2cc(Cl)nnc2n1C[C@@H]1CCO1 ; Fc1cc(Cl)ccc1COc1nc(C2CCN(Cc3nc4cc(Cl)nnc4n3C[C@@H]3CCO3)CC2)ccc1F ; Fc1cc(Cl)ccc1COc1nc(C2CCN(Cc3nc4cc(-c5nnc(C(F)(F)F)[nH]5)nnc4n3C[C@@H]3CCO3)CC2)ccc1F]

### Step A: (S)-3-chloro-6-((4-(6-((4-chloro-2-fluorobenzyl)oxy)-5-fluoropyridin-2-yl)piperidin-1-yl)methyl)-7-(oxetan-2-ylmethyl)-7H-imidazo[4,5-c]pyridazine

To a solution of 2-((4-chloro-2-fluorobenzyl)oxy)-3-fluoro-6-(piperidin-4-yl)pyridine TFA Salt (440 mg, crude) in DMF (4 mL) was added DIEA (554 mg, 4.3 mmol) and (S)-3-chloro-6-(chloromethyl)-7-(oxetan-2-ylmethyl)-7H-imidazo[4,5-c]pyridazine (235 mg, 0.86 mmol). The solution was stirred at 50° C. for 3 hours. The reaction was quenched with H₂O (50 mL), extracted with ethyl acetate (70 mL×2). The organic layer was dried over anhydrous sodium sulfate, filtered and evaporated to dryness. The residue was purified by column chromatography on silica gel (PE/EA=1/1) to give (S)-3-chloro-6-((4-(6-((4-chloro-2-fluorobenzyl)oxy)-5-fluoropyridin-2-yl)piperidin-1-yl)methyl)-7-(oxetan-2-ylmethyl)-7H-imidazo[4,5-c]pyridazine (274 mg, 55% yield) as a yellow solid. MS Calcd.: 574.2; MS Found: 575.3 [M+H]⁺.

### Step B: 2-[(4-chloro-2-fluorophenyl)methoxy]-3-fluoro-6-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyridine (Compound 488a)

2-[(4-chloro-2-fluorophenyl)methoxy]-3-fluoro-6-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyridine (Compound 488a) (5.0 mg) was obtained as a white solid.

MS Calcd.: 675.2; MS Found: 676.0 [M+H]⁺.

¹H NMR (400 MHz, DMSO-d₆): δ 8.48 (s, 1H), 7.56-7.63 (m, 2H), 7.48 (dd, J=10.0 Hz, 2.0 Hz, 1H), 7.32 (dd, J=8.0 Hz, 1.6 Hz, 1H), 6.92 (dd, J=8.4 Hz, 2.8 Hz, 1H), 5.47 (s, 2H), 5.26-5.33 (m, 1H), 5.00-5.08 (m, 1H), 4.87-4.95 (m, 1H), 4.38-4.55 (m, 2H), 4.01-4.09 (m, 2H), 2.94-3.04 (m, 2H), 2.68-2.81 (m, 1H), 2.52-2.68 (m, 2H), 2.25-2.35 (m, 2H), 1.67-1.85 (m, 4H). ¹⁹F NMR (377 MHz, DMSO-d₆): δ −63.12, −115.00, −144.00.

### Example 294: 4-[(4-chloro-2-fluorophenyl)methoxy]-5-fluoro-2-[(3S)-3-methyl-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazin-1-yl]pyrimidine (Compound 489a)

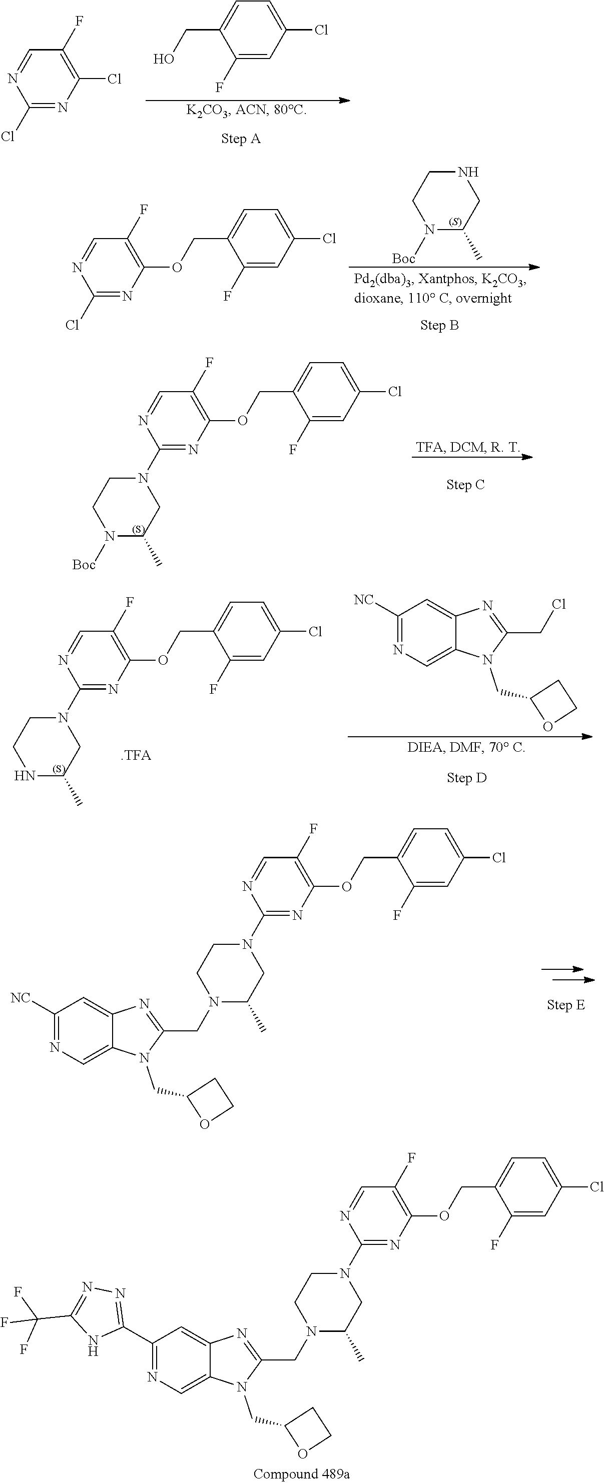

### Step A: 2-chloro-4-((4-chloro-2-fluorobenzyl)oxy)-5-fluoropyrimidine

A mixture of 2,4-dichloro-5-fluoropyrimidine (25.0 g, 150 mmol), (4-chloro-2-fluorophenyl)methanol (28.9 g, 180 mmol), K₂CO₃ (62.1 g, 450 mmol) in ACN (200 mL) was stirred at 80° C. overnight. The resulting mixture was filtered, the filtrate was concentrated and purified by column chromatography on silica gel (PE/EA=50/1) to give 2-chloro-4-((4-chloro-2-fluorobenzyl)oxy)-5-fluoropyrimidine (38.3 g, 88% yield) as a white solid. MS Calcd.: 290.0; MS Found: 291.2 [M+H]⁺.

### Step B: tert-butyl (S)-4-(4-((4-chloro-2-fluorobenzyl)oxy)-5-fluoropyrimidin-2-yl)-2-methylpiperazine-1-carboxylate

A mixture of 2-chloro-4-((4-chloro-2-fluorobenzyl)oxy)-5-fluoropyrimidine (1.0 g, 3.4 mmol), tert-butyl (S)-2-methylpiperazine-1-carboxylate (830 mg, 4.1 mmol), Xantphos (200 mg, 0.34 mmol), Pd₂(dba)₃ (315 mg, 0.34 mmol), K₂CO₃ (1.43 g, 10.4 mmol) in dioxane (40 mL) was stirred at 110° C. overnight. The resulting mixture was concentrated and purified by column chromatography on silica gel (PE/EA=10/1) to give tert-butyl (S)-4-(4-((4-chloro-2-fluorobenzyl)oxy)-5-fluoropyrimidin-2-yl)-2-methylpiperazine-1-carboxylate (500 mg, 32% yield) as yellow oil. MS Calcd.: 454.2; MS Found: 455.1 [M+H]⁺.

### Step C: (S)-4-((4-chloro-2-fluorobenzyl)oxy)-5-fluoro-2-(3-methylpiperazin-1-yl)pyrimidine. TFA salt

A mixture of tert-butyl (S)-4-(4-((4-chloro-2-fluorobenzyl)oxy)-5-fluoropyrimidin-2-yl)-2-methylpiperazine-1-carboxylate (260 mg, 0.57 mmol), TFA (1 mL) in DCM (4 mL) was stirred at room temperature for 1 h. After the reaction was completed, the mixture was concentrated in vacuum to give (S)-4-((4-chloro-2-fluorobenzyl)oxy)-5-fluoro-2-(3-methylpiperazin-1-yl)pyrimidine. TFA salt (crude 348 mg) as yellow oil. MS Calcd.: 354.1; MS Found: 355.1 [M+H]⁺.

### Step D: 2-(((S)-4-(4-((4-chloro-2-fluorobenzyl)oxy)-5-fluoropyrimidin-2-yl)-2-methylpiperazin-1-yl)methyl)-3-(((S)-oxetan-2-yl)methyl)-3H-imidazo[4,5-c]pyridine-6-carbonitrile

A mixture of (S)-4-((4-chloro-2-fluorobenzyl)oxy)-5-fluoro-2-(3-methylpiperazin-1-yl)pyrimidine. TFA salt (crude 348 mg, 0.57 mmol), (S)-2-(chloromethyl)-3-(oxetan-2-ylmethyl)-3H-imidazo[4,5-c]pyridine-6-carbonitrile (150 mg, 0.57 mmol), DIEA (738 mg, 5.7 mmol) in DMF (4 mL) was stirred at 70° C. for 5 h. After the reaction was completed, the mixture was concentrated and the residue was purified by column chromatography on silica gel (EA/PE=2/1) to give 2-(((S)-4-(4-((4-chloro-2-fluorobenzyl)oxy)-5-fluoropyrimidin-2-yl)-2-methylpiperazin-1-yl)methyl)-3-(((S)-oxetan-2-yl)methyl)-3H-imidazo[4,5-c]pyridine-6-carbonitrile (219 mg, 66% yield, two steps) as a yellow oil. MS Calcd.: 580.2; MS Found: 581.3 [M+H]⁺.

### Step E: 4-[(4-chloro-2-fluorophenyl)methoxy]-5-fluoro-2-[(3S)-3-methyl-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazin-1-yl]pyrimidine (Compound 489a)

4-[(4-chloro-2-fluorophenyl)methoxy]-5-fluoro-2-[(3S)-3-methyl-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazin-1-yl]pyrimidine (Compound 489a) (16 mg) was obtained as a white solid.

MS Calcd.: 690.2; MS Found: 691.1 [M+H]⁺.

¹H NMR (400 MHz, DMSO-d₆): δ 9.17 (s, 1H), 8.28 (s, 1H), 8.20 (d, J=2.8 Hz, 1H), 7.58 (t, J=8.0 Hz, 1H), 7.50 (d, J=10.0 Hz, 1H), 7.33 (d, J=7.6 Hz, 1H), 5.47 (s, 2H), 5.16-5.27 (m, 1H), 4.80-4.94 (m, 2H), 4.46-4.55 (m, 1H), 4.31-4.41 (m, 2H), 4.07 (d, J=11.2 Hz, 1H), 3.99 (d, J=12.0 Hz, 1H), 3.77 (d, J=13.6 Hz, 1H), 3.19-3.26 (m, 1H), 3.04-3.14 (m, 1H), 2.59-2.79 (m, 3H), 2.29-2.51 (m, 2H), 1.11 (d, J=6.0 Hz, 3H). ¹⁹F NMR (377 MHz, DMSO-d₆): δ −63.56, −114.84, −171.94.

### Example 295: 4-[(4-chloro-2-fluorophenyl)methoxy]-5-fluoro-2-{4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazin-1-yl}pyrimidine (Compound 490a)

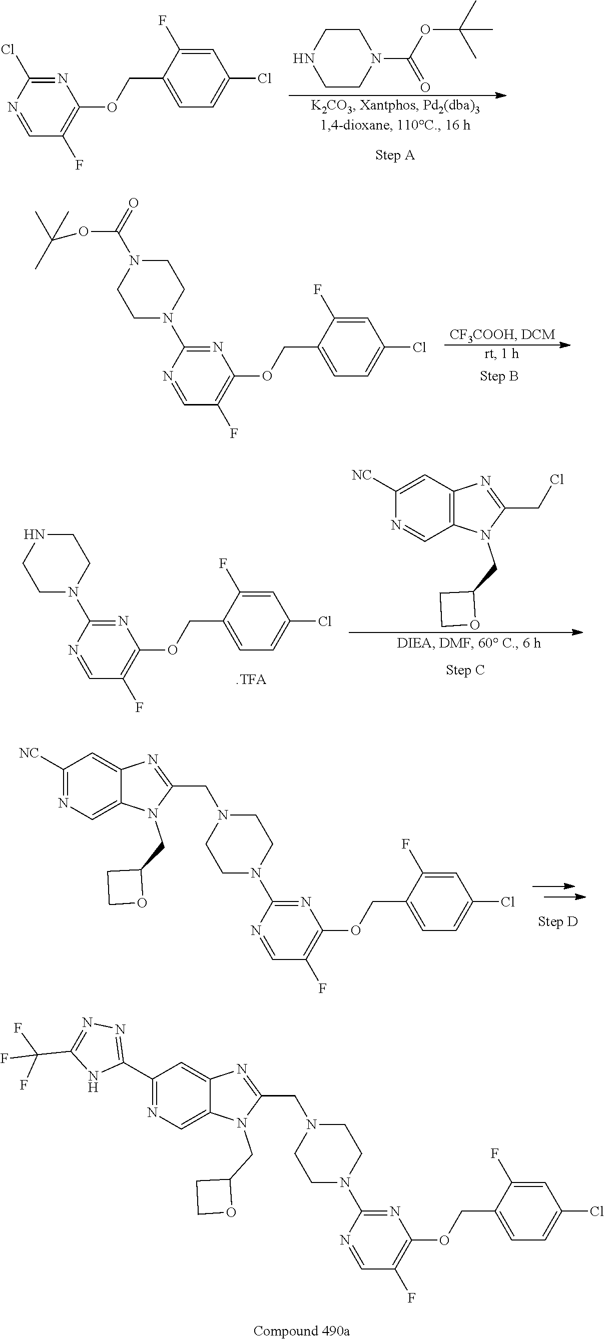

### Step A: tert-butyl 4-(4-((4-chloro-2-fluorobenzyl)oxy)-5-fluoropyrimidin-2-yl)piperazine-1-carboxylate

A mixture of 2-chloro-4-((4-chloro-2-fluorobenzyl)oxy)-5-fluoropyrimidine (500 mg, 1.72 mmol). tert-butyl piperazine-1-carboxylate (383 mg, 2.06 mmol), K₂CO₃ (712 mg, 5.16 mmol), Xantphos (100 mg, 0.17 mmol) and Pd₂(dba)₃ (158 mg, 0.172 mmol) in dioxane (10 mL) was degassed and charged with N₂. The reaction mixture was stirred at 110° C. for 16 hours. After the reaction was completed, the mixture was diluted with EA (30 mL), washed with H₂O (20 mL*2). The combined organic layer was dried over anhydrous sodium sulfate, filtered and evaporated to dryness. The residue was purified by column chromatography on silica gel (PE/EA=10/1) to give tert-butyl 4-(4-((4-chloro-2-fluorobenzyl)oxy)-5-fluoropyrimidin-2-yl)piperazine-1-carboxylate (512 mg, yield: 67%) as yellow oil. MS Calcd.: 440.1; MS Found: 385.1 [M-56+1]⁺.

### Step B: 4-((4-chloro-2-fluorobenzyl)oxy)-5-fluoro-2-(piperazin-1-yl)pyrimidine. TFA salt

To a solution of tert-butyl 4-(4-((4-chloro-2-fluorobenzyl)oxy)-5-fluoropyrimidin-2-yl)piperazine-1-carboxylate (185 mg, 0.42 mmol) in DCM (2 mL) was added TFA (0.75 mL). The mixture was stirred at room temperature for 1 hour. After the reaction was completed, the mixture was concentrated to give 4-((4-chloro-2-fluorobenzyl)oxy)-5-fluoro-2-(piperazin-1-yl)pyrimidine. TFA salt (343 mg, crude) as yellow oil. MS Calcd.: 340.1; MS Found: 340.9 [M+H]⁺.

### Step C: (S)-2-((4-(4-((4-chloro-2-fluorobenzyl)oxy)-5-fluoropyrimidin-2-yl)piperazin-1-yl)methyl)-3-(oxetan-2-ylmethyl)-3H-imidazo[4,5-c]pyridine-6-carbonitrile

To a solution of 4-((4-chloro-2-fluorobenzyl)oxy)-5-fluoro-2-(piperazin-1-yl)pyrimidine. TFA salt (crude, 343 mg) in DMF (2 mL) was added DIEA (30 mg, 2.28 mmol). After 2 minutes, (S)-2-(chloromethyl)-3-(oxetan-2-ylmethyl)-3H-imidazo[4,5-c]pyridine-6-carbonitrile (100 mg, 0.36 mmol) was added. The mixture was stirred at 60° C. for 6 hours. After the reaction was completed, the mixture was diluted with EA (30 mL), washed with brine (20 mL×3). The organic layer was dried over anhydrous sodium sulfate, filtered and evaporated to dryness. The residue was purified by column chromatography on silica gel (PE/EA=15/1) to give (S)-2-((4-(4-((4-chloro-2-fluorobenzyl)oxy)-5-fluoropyrimidin-2-yl)piperazin-1-yl)methyl)-3-(oxetan-2-ylmethyl)-3H-imidazo[4,5-c]pyridine-6-carbonitrile (204 mg, 85% yield, two steps) as a yellow solid. MS Calcd.: 566.2; MS Found: 566.9 [M+H]⁺.

### Step D: 4-[(4-chloro-2-fluorophenyl)methoxy]-5-fluoro-2-{4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazin-1-yl}pyrimidine (Compound 490a)

4-[(4-chloro-2-fluorophenyl)methoxy]-5-fluoro-2-{4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazin-1-yl}pyrimidine (Compound 490a) (47 mg) was obtained.

MS Calcd.: 676.2; MS Found: 677.3 [M+H]⁺.

¹H NMR (400 MHz, DMSO-d₆): δ 9.28 (d, J=0.8 Hz, 1H), 8.43 (d, J=1.2 Hz, 1H), 8.28 (d, J=2.8 Hz, 1H), 7.61 (t, J=8.0 Hz, 1H), 7.49 (dd, J=2.0 Hz, J=10.0 Hz, 1H), 7.35 (dd, J=2.0 Hz, J=8.0 Hz, 1H), 5.51 (s, 2H), 5.07-5.15 (m, 1H), 4.69-4.93 (m, 4H), 4.49-4.56 (m, 1H), 4.37-4.44 (m, 1H), 3.87-4.09 (m, 4H), 3.28-3.45 (m, 4H), 2.70-2.82 (m, 1H), 2.33-2.43 (m, 1H). ¹⁹F NMR (377 MHz, DMSO-d₆): δ −63.76, −114.56, −170.11.

### Example 296: 4-[(4-chloro-2-fluorophenyl)methoxy]-5-fluoro-2-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyrimidine (Compound 491a)

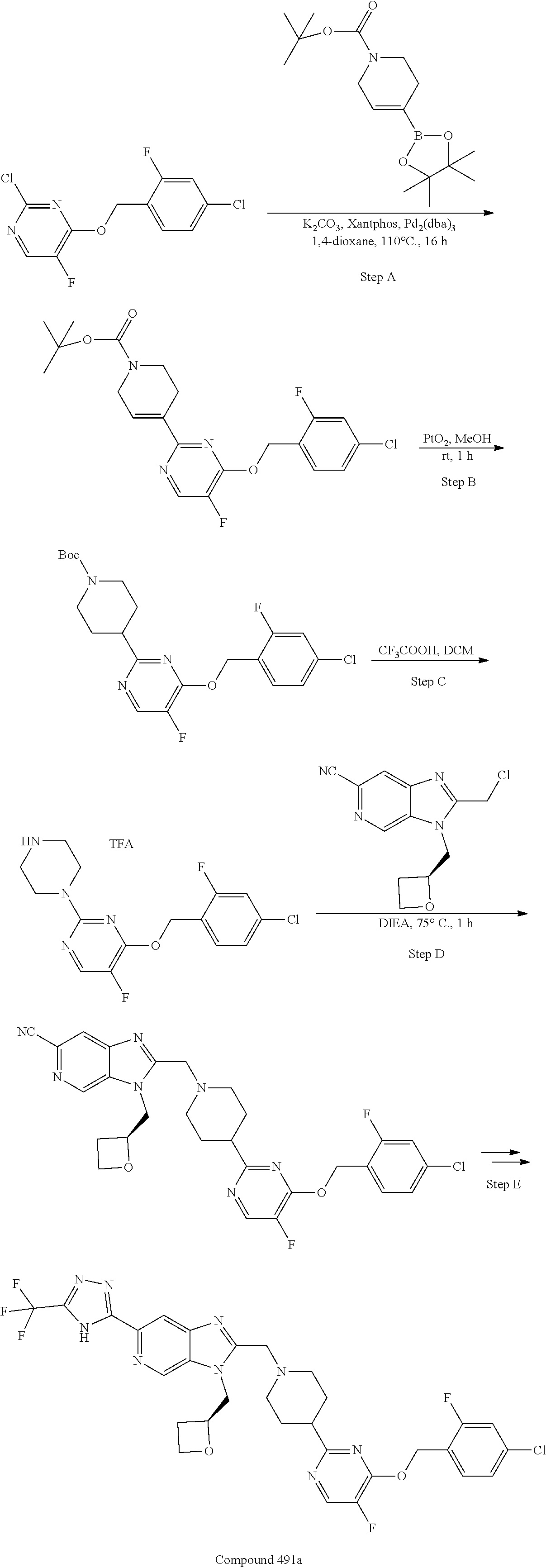

### Step A: tert-butyl 4-(4-((4-chloro-2-fluorobenzyl)oxy)-5-fluoropyrimidin-2-yl)-3,6-dihydropyridine-1(2H)-carboxylate

A mixture of 2-chloro-4-((4-chloro-2-fluorobenzyl)oxy)-5-fluoropyrimidine (3.0 g, 10.34 mmol). tert-butyl 4-(4,4,5,5-tetramethyl-1,3,2-dioxaborolan-2-yl)-3,6-dihydropyridine-1(2H)-carboxylate (3.8 g, 12.4 mmol), K₂CO₃ (4.3 g, 31.0 mmol) and Pd(dppf)Cl₂DCM (422 mg, 0.52 mmol) in dioxane (60 mL) and H₂O (2 mL) was degassed and charged with N₂. The reaction was stirred at 90° C. for overnight. After the reaction was completed, the mixture was evaporated to dryness. The residue was purified by column chromatography on silica gel (PE/EA=15/1) to give tert-butyl 4-(4-((4-chloro-2-fluorobenzyl)oxy)-5-fluoropyrimidin-2-yl)-3,6-dihydropyridine-1(2H)-carboxylate (3.317 g, yield: 73%) as a colourless oil. MS Calcd.: 437.1; MS Found: 438.1 [M+1]⁺.

### Step B: tert-butyl 4-(4-((4-chloro-2-fluorobenzyl)oxy)-5-fluoropyrimidin-2-yl)piperidine-1-carboxylate

A mixture of tert-butyl 4-(4-((4-chloro-2-fluorobenzyl)oxy)-5-fluoropyrimidin-2-yl)-3,6-dihydropyridine-1(2H)-carboxylate (3.3 g, 7.6 mmol) and PtO₂ (1.0 g, 4.4 mmol) in MeOH (30 mL) was degassed and charged with H₂. The reaction was stirred at rt for 1 h. After the reaction was completed, the mixture was filtered and evaporated to dryness. The residue was purified by column chromatography on silica gel (PE/EA=15/1) to give tert-butyl 4-(4-((4-chloro-2-fluorobenzyl)oxy)-5-fluoropyrimidin-2-yl)piperidine-1-carboxylate (1.9 g, yield: 58%) as a colourless oil. MS Calcd.: 439.2; MS Found: 384.1 [M-56+1]⁺.

### Step C: 4-((4-chloro-2-fluorobenzyl)oxy)-5-fluoro-2-(piperidin-4-yl)pyrimidine TFA salt

A solution of tert-butyl 4-(4-((4-chloro-2-fluorobenzyl)oxy)-5-fluoropyrimidin-2-yl)piperidine-1-carboxylate (300 mg, 0.68 mmol), TFA (0.5 mL) in DCM (3 mL) was stirred at room temperature for 1 h. Upon completion, the reaction mixture was concentrated to give 4-((4-chloro-2-fluorobenzyl)oxy)-5-fluoro-2-(piperidin-4-yl)pyrimidine TFA salt (350 mg, crude) as yellow oil.

### Step D: (S)-2-((4-(4-((4-chloro-2-fluorobenzyl)oxy)-5-fluoropyrimidin-2-yl)piperidin-1-yl)methyl)-3-(oxetan-2-ylmethyl)-3H-imidazo[4,5-c]pyridine-6-carbonitrile

A mixture of 4-(3-((4-chloro-2-fluorobenzyl)oxy)-4-fluorophenyl)piperidine TFA salt (350 mg, crude), (S)-2-(chloromethyl)-3-(oxetan-2-ylmethyl)-3H-imidazo[4,5-c]pyridine-6-carbonitrile (180 mg, 0.68 mmol), DIEA (356 mg, 2.7 mmol) in DMF (3 mL) was stirred at 75° C. for 1 hour. After the reaction was completed, the mixture was diluted with H₂O (20 mL) and extracted with EA (50 mL×2). The combined organic layer was washed by brine (30 mL×3) and dried over anhydrous sodium sulfate, filtered and evaporated to dryness. The residue was purified by column chromatography on silica gel (DCM/MeOH=10/1) to give (S)-2-((4-(4-((4-chloro-2-fluorobenzyl)oxy)-5-fluoropyrimidin-2-yl)piperidin-1-yl)methyl)-3-(oxetan-2-ylmethyl)-3H-imidazo[4,5-c]pyridine-6-carbonitrile (320 mg, 82% yield) as a yellow solid. MS Calcd.: 565.2; MS Found: 566.2[M+H]⁺.

### Step E: 4-[(4-chloro-2-fluorophenyl)methoxy]-5-fluoro-2-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyrimidine (Compound 491a)

4-[(4-chloro-2-fluorophenyl)methoxy]-5-fluoro-2-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyrimidine (Compound 491a) (100 mg) was obtained as a white solid.

MS Calcd.: 675.2; MS Found: 676.2[M+H]⁺.

¹H NMR (400 MHz, DMSO-d₆): δ 9.17 (s, 1H), 8.56 (d, J=2.8 Hz, 1H), 8.29 (s, 1H), 7.62 (t, J=8.4 Hz, 1H), 7.51 (dd, J=2.0, J=10.0 Hz, 1H), 7.34 (dd, J=2.0, J=8.4 Hz, 1H), 5.55 (s, 2H), 5.11-5.23 (m, 1H), 4.88-4.99 (m, 1H), 4.75-4.80 (m, 1H), 4.39-4.55 (m, 2H), 4.01 (d, J=13.6 Hz, 1H), 3.86 (d, J=14.0 Hz, 1H), 2.95-3.04 (m, 1H), 2.82-2.90 (m, 1H), 2.65-2.80 (m, 2H), 2.42-2.53 (m, 1H), 2.19-2.35 (m, 2H), 1.86-2.03 (m, 2H), 1.65-1.85 (m, 2H). ¹⁹F NMR (377 MHz, DMSO-d₆): δ −63.72, −114.71, −157.66.

### Example 297: 1-{6-[(4-chloro-2-fluorophenyl)methoxy]pyridin-2-yl}-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 492a)

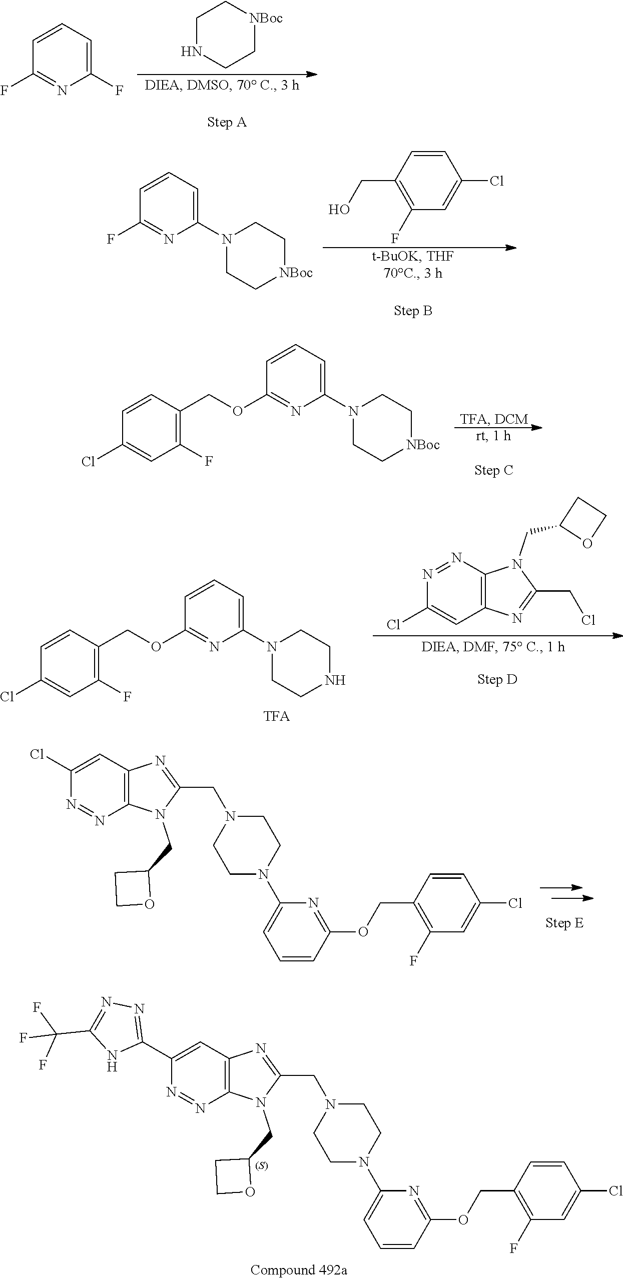

### Step A: tert-butyl 4-(6-fluoropyridin-2-yl)piperazine-1-carboxylate

To a solution of 2,6-difluoropyridine (5.0 g, 43.5 mmol) in DMSO (60 mL) was added tert-butyl piperazine-1-carboxylate (8.9 g, 47.8 mmol) and DIEA (8.41 g, 65.217 mmol). The mixture was stirred at 70° C. for 3 hours. The mixture was cooled down to room temperature, diluted with ethyl acetate (150 mL), washed with H₂O (200 mL*2). The organic layer was dried over anhydrous sodium sulfate, filtered and evaporated to dryness. The residue was purified by column chromatography on silica gel (PE/EA=8/1) to give to give tert-butyl 4-(6-fluoropyridin-2-yl)piperazine-1-carboxylate (9.3 g, yield: 76%) as colorless oil. MS Calcd.: 281.2; MS Found: 304.1 [M+23]⁺.

### Step B: tert-butyl 4-(6-((4-chloro-2-fluorobenzyl)oxy)pyridin-2-yl)piperazine-1-carboxylate

To a solution of tert-butyl 4-(6-fluoropyridin-2-yl)piperazine-1-carboxylate (3.0 g, 10.7 mmol) in THE (20 mL) was added (4-chloro-2-fluorophenyl)methanol (2.1 g, 12.8 mmol) and t-BuOK (21.3 mL, 21.352 mmol, 1M solution in THF) at rt. The mixture was stirred at 70° C. for 3 hours. The mixture was cooled to room temperature, diluted with ethyl acetate (100 mL), washed with H₂O (200 mL*2). The organic layer was dried over anhydrous sodium sulfate, filtered and evaporated to dryness. The residue was purified by column chromatography on silica gel (PE/EA=10/1) to give to give tert-butyl 4-(6-((4-chloro-2-fluorobenzyl)oxy)pyridin-2-yl)piperazine-1-carboxylate (4.2 g, yield: 93%) as colorless oil. MS Calcd.: 421.2; MS Found: 422.2 [M+H]⁺.

### Step C: 1-(6-((4-chloro-2-fluorobenzyl)oxy)pyridin-2-yl)piperazine TFA salt

To a solution of tert-butyl 4-(6-((4-chloro-2-fluorobenzyl)oxy)pyridin-2-yl)piperazine-1-carboxylate (240 mg, 0.57 mmol) in DCM (4 mL) was added TFA (0.5 mL). The solution was stirred at room temperature for an hour. The solvent was removed in vacuo to give 1-(6-((4-chloro-2-fluorobenzyl)oxy)pyridin-2-yl)piperazine TFA salt (258 mg, crude) as brown oil. MS Calcd.: 321.1; MS Found: 322.0 [M+H]⁺.

### Step D: (S)-3-chloro-6-((4-(6-((4-chloro-2-fluorobenzyl)oxy)pyridin-2-yl)piperazin-1-yl)methyl)-7-(oxetan-2-ylmethyl)-7H-imidazo[4,5-c]pyridazine

To a solution of 1-(6-((4-chloro-2-fluorobenzyl)oxy)pyridin-2-yl)piperazine (703.7 mg, crude, TFA salt) in DMF (8 mL) was added DIEA (569 mg, 4.4 mmol). After 2 minutes, (S)-3-chloro-6-(chloromethyl)-7-(oxetan-2-ylmethyl)-7H-imidazo[4,5-c]pyridazine (400 mg, 1.5 mmol) was added. The resulting mixture was stirred at 75° C. for an hour. After the reaction was completed, the mixture was diluted with EA (100 mL), washed with H₂O (50 mL*2). The organic layer was dried over anhydrous sodium sulfate, filtered and evaporated to dryness. The residue was purified by column chromatography on silica gel (DCM/MeOH=30/1) to give (S)-3-chloro-6-((4-(6-((4-chloro-2-fluorobenzyl)oxy)pyridin-2-yl)piperazin-1-yl)methyl)-7-(oxetan-2-ylmethyl)-7H-imidazo[4,5-c]pyridazine (780 mg, yield: 95%) as brown oil. MS Calcd.: 557.2; MS Found: 558.3 [M+H]⁺.

### Step E: 1-{6-[(4-chloro-2-fluorophenyl)methoxy]pyridin-2-yl}-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 492a)

1-{6-[(4-chloro-2-fluorophenyl)methoxy]pyridin-2-yl}-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 492a) (10 mg) was obtained as a white solid.

MS Calcd.: 658.2; MS Found: 659.0 [M+H]⁺.

¹H NMR (400 MHz, DMSO-d₆): δ 8.64 (s, 1H), 7.50-7.58 (m, 2H), 7.45 (dd, J=10.0 Hz, 1.6 Hz, 1H), 7.30 (d, J=8.0 Hz, 1H), 6.47 (d, J=8.0 Hz, 1H), 6.20 (d, J=7.6 Hz, 1H), 5.33 (s, 2H), 5.17-5.25 (m, 1H), 4.95-5.03 (m, 1H), 4.75-4.93 (m, 3H), 4.49-4.56 (m, 1H), 4.30-4.40 (m, 1H), 3.12-3.44 (m, 8H), 2.70-2.81 (m, 1H), 2.42-2.52 (m, 1H). ¹⁹F NMR (377 MHz, DMSO-d₆): δ −63.67, −115.33.

### Example 298: 3-fluoro-4-{[(6-{4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazin-1-yl}pyridin-2-yl)oxy]methyl}benzonitrile (Compound 493a)

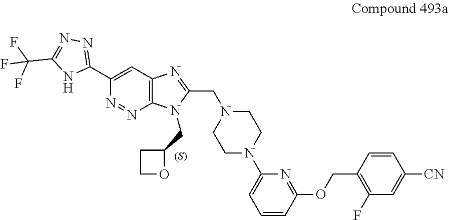

3-fluoro-4-{[(6-{4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazin-1-yl}pyridin-2-yl)oxy]methyl}benzonitrile (Compound 493a) (5.0 mg) was obtained as a white solid.

MS Calcd.: 649.2; MS Found: 650.1 [M+H]⁺.

¹H NMR (400 MHz, DMSO-d₆): δ 8.60 (s, 1H), 7.87 (dd, J=10.0 Hz, 1.2 Hz, 1H), 7.62-7.73 (m, 2H), 7.54 (t, J=8.0 Hz, 1H), 6.43 (d, J=8.0 Hz, 1H), 6.20 (d, J=8.0 Hz, 1H), 5.42 (s, 2H), 5.15-5.27 (m, 1H), 4.95-5.05 (m, 1H), 4.82-4.90 (m, 1H), 4.43-4.80 (m, 3H), 4.32-4.40 (m, 1H), 2.80-3.39 (m, 8H), 2.50-2.80 (m, 2H). ¹⁹F NMR (377 MHz, DMSO-d₆): δ −63.67, −115.65.

### Example 299: 1-{6-[(4-chloro-2-fluorophenyl)methoxy]pyridin-2-yl}-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazine (Compound 494a)

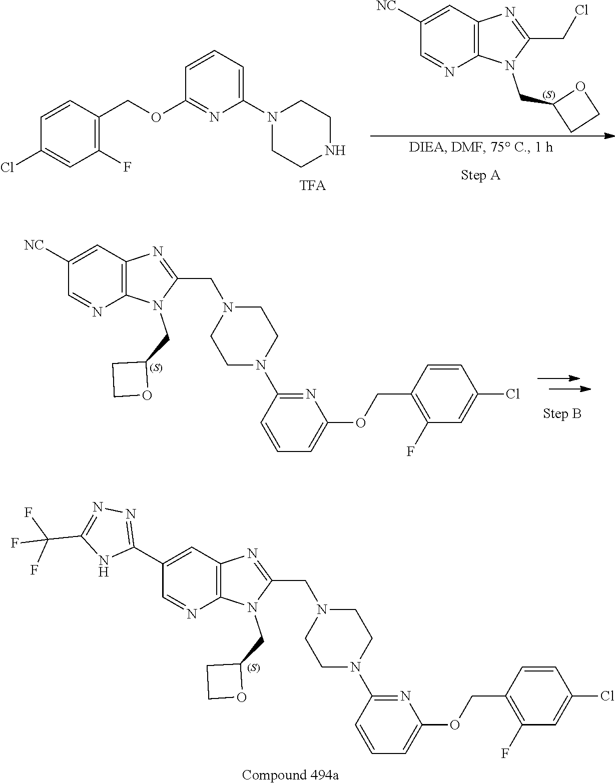

### Step A: (S)-2-((4-(6-((4-chloro-2-fluorobenzyl)oxy)pyridin-2-yl)piperazin-1-yl)methyl)-3-(oxetan-2-ylmethyl)-3H-imidazo[4,5-b]pyridine-6-carbonitrile

To a solution of 1-(6-((4-chloro-2-fluorobenzyl)oxy)pyridin-2-yl)piperazine (274 mg, crude, TFA salt) in DMF (4 mL) was added DIEA (147 mg, 1.144 mmol). After 2 minutes, (S)-2-(chloromethyl)-3-(oxetan-2-ylmethyl)-3H-imidazo[4,5-b]pyridine-6-carbonitrile (150 mg, 0.572 mmol) was added. The resulting mixture was heated to 75° C. for an hour. The reaction was quenched with water (20 mL), extracted with ethyl acetate (30 mL×2). The organic layers were combined, dried over Na₂SO₄, filtered and concentrated under vacuum. The residue was purified by flash chromatography (DCM:MeOH=30:1) to give (S)-2-((4-(6-((4-chloro-2-fluorobenzyl)oxy)pyridin-2-yl)piperazin-1-yl)methyl)-3-(oxetan-2-ylmethyl)-3H-imidazo[4,5-b]pyridine-6-carbonitrile (220 mg, 70% yield) as a brown solid. MS Calcd.: 547.2; MS Found: 548.2 [M+H]⁺.

### Step B: 1-{6-[(4-chloro-2-fluorophenyl)methoxy]pyridin-2-yl}-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazine (Compound 494a)

1-{6-[(4-chloro-2-fluorophenyl)methoxy]pyridin-2-yl}-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazine (Compound 494a) (29 mg) was obtained as a white solid.

MS Calcd.: 657.2; MS Found: 658.0 [M+H]⁺.

¹H NMR (400 MHz, DMSO-d₆): δ 9.00 (d, J=2.0 Hz, 1H), 8.58 (d, J=2.0 Hz, 1H), 7.42-7.55 (m, 3H), 7.29 (dd, J=8.0 Hz, 2.0 Hz, 1H), 6.34 (d, J=8.0 Hz, 1H), 6.09 (d, J=8.0 Hz, 1H), 5.30 (s, 2H), 5.15-5.25 (m, 1H), 4.80-4.90 (m, 1H), 4.69-4.78 (m, 1H), 4.47-4.52 (m, 1H), 4.35-4.41 (m, 1H), 3.90-4.02 (m, 2H), 3.40-3.53 (m, 4H), 2.65-2.75 (m, 1H), 2.53-2.62 (m, 4H), 2.42-2.53 (m, 1H). ¹⁹F NMR (377 MHz, DMSO-d₆): δ −63.18, −115.42.

### Example 300: 3-fluoro-4-{[(6-{4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazin-1-yl}pyridin-2-yl)oxy]methyl}benzonitrile (Compound 495a)

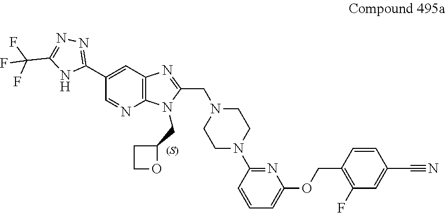

3-fluoro-4-{[(6-{4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazin-1-yl}pyridin-2-yl)oxy]methyl}benzonitrile (Compound 495a) (13 mg) was obtained as a white solid.

MS Calcd.: 648.2; MS Found: 649.1 [M+H]⁺.

¹H NMR (400 MHz, DMSO-d₆): δ 9.00 (d, J=2.0 Hz, 1H), 8.58 (d, J=2.0 Hz, 1H), 7.87 (d, J=10.0 Hz, 1.2 Hz, 1H), 7.63-7.71 (m, 2H), 7.48 (t, J=8.0 Hz, 2.0 Hz, 1H), 6.34 (d, J=8.4 Hz, 1H), 6.12 (d, J=8.0 Hz, 1H), 5.40 (s, 2H), 5.15-5.23 (m, 1H), 4.80-4.88 (m, 1H), 4.69-4.75 (m, 1H), 4.45-4.52 (m, 1H), 4.33-4.42 (m, 1H), 3.90-4.02 (m, 2H), 3.40-3.49 (m, 4H), 2.60-2.75 (m, 1H), 2.52-2.61 (m, 4H), 2.40-2.52 (m, 1H). ¹⁹F NMR (377 MHz, DMSO-d₆): δ −63.24, −115.71.

### Example 301: 2-[(2-fluoro-4-methylphenyl)methoxy]-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 496a)

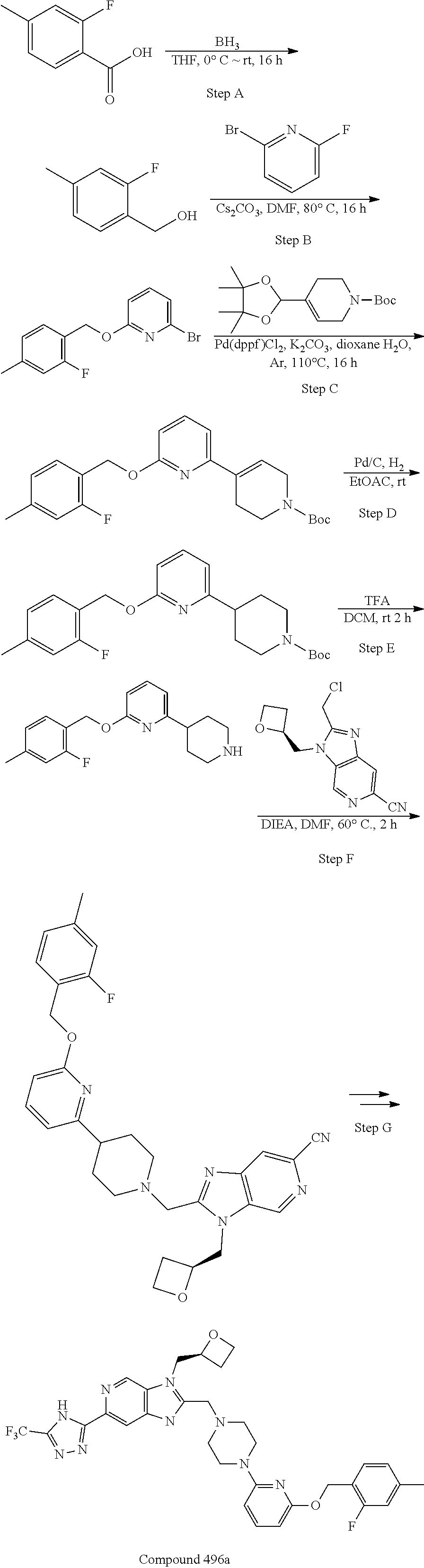

### Step A: (2-fluoro-4-methylphenyl)methanol

To a solution of 2-fluoro-4-methylbenzoic acid (1000 mg, 6.5 mmol) in THF (15 mL) were added dropwise BH₃·THF (1 M) (19.5 ml, 19.5 mmol) at 0° C. under Ar. The mixture was stirred at rt for 16 h. The mixture was added NH₄C₁.aq (100 mL) and extracted with EtOAC (2×100 mL). The combined organic layer was dried over sodium sulfate was concentrated to obtain (2-fluoro-4-methylphenyl)methanol (1300 mg, crude) as a yellow oil.

### Step B: 2-bromo-6-((2-fluoro-4-methylbenzyl)oxy)pyridine

A mixture of (2-fluoro-4-methylphenyl)methanol (300 mg, crude), 2-bromo-6-fluoropyridine (563 mg, 3.2 mmol) and Cs₂CO₃ (2095 mg, 6.4 mmol) in DMF (30 mL) was stirred at 80° C. for 16 h. The mixture was poured into water (100 mL) and extracted with EtOAc (2×100 mL), the combined organic layer was washed with brine, dried over sodium sulfate, filtered and the residue was concentrated. The residue was purified by column chromatography on silica gel to obtain 2-bromo-6-((2-fluoro-4-methylbenzyl)oxy)pyridine (320 mg, yield: 50%) as a yellow solid. MS Calcd: 295.0; MS Found: 295.9 [M+H]⁺.

### Step C: tert-butyl 6-((2-fluoro-4-methylbenzyl)oxy)-3′,6′-dihydro-[2,4′-bipyridine]-1′(2′H)-carboxylate

A mixture of 2-bromo-6-((2-fluoro-4-methylbenzyl)oxy)pyridine (320 mg, 1.1 mmol), tert-butyl 4-(4,4,5,5-tetramethyl-1,3-dioxolan-2-yl)-3,6-dihydropyridine-1(2H)-carboxylate (1012 mg, 3.2 mmol), Pd(dppf)Cl₂ (159 mg, 0.22 mmol) and K₂CO₃ (599 mg, 4.3 mmol) in dioxane/water (20 mL/2 mL) was stirred at 110° C. under Ar for 16 h. The mixture was poured into water (100 mL) and extracted with EtOAc (2×100 mL), the combined organic layer was washed with brine, dried over sodium sulfate, filtered and the residue was concentrated. The residue was purified by column chromatography on silica gel to obtain tert-butyl 6-((2-fluoro-4-methylbenzyl)oxy)-3′,6′-dihydro-[2,4′-bipyridine]-1′(2′H)-carboxylate (920 mg, crude) as a clear oil. MS Calcd: 398.2; MS Found: 399.6 [M+H]⁺.

### Step D: tert-butyl 4-(6-((2-fluoro-4-methylbenzyl)oxy)pyridin-2-yl)piperidine-1-carboxylate

A mixture of tert-butyl 6-((2-fluoro-4-methylbenzyl)oxy)-3′,6′-dihydro-[2,4′-bipyridine]-1′(2′H)-carboxylate (920 mg, crude) and Pd/C (25 mg, 10% w/w) in EtOAc (10 mL) was stirred at rt under H₂ for 16 h. The mixture was filtered and the filtrate was concentrated to obtain tert-butyl 4-(6-((2-fluoro-4-methylbenzyl)oxy)pyridin-2-yl)piperidine-1-carboxylate (900 mg, yield: 97%) as a clear oil. MS Calcd: 400.2; MS Found: 401.2 [M+H]⁺.

### Step E: 2-((2-fluoro-4-methylbenzyl)oxy)-6-(piperidin-4-yl)pyridine

A mixture of tert-butyl 4-(6-((2-fluoro-4-methylbenzyl)oxy)pyridin-2-yl)piperidine-1-carboxylate (900 mg, 2.2 mmol) and TFA (2 mL) in DCM (2 mL) was stirred at rt for 2 h. The mixture was poured into NaHCO₃ aqueous solution (50 mL) and extracted with DCM (2×50 mL), the combined organic layer was washed with brine, dried over sodium sulfate, filtered and the residue was concentrated to obtain 2-((2-fluoro-4-methylbenzyl)oxy)-6-(piperidin-4-yl)pyridine (160 mg, yield: 24%) as a yellow oil. MS Calcd: 300.2; MS Found: 301.1 [M+H]⁺.

### Step F: (S)-2-((4-(6-((2-fluoro-4-methylbenzyl)oxy)pyridin-2-yl)piperidin-1-yl)methyl)-3-(oxetan-2-ylmethyl)-3H-imidazo[4,5-c]pyridine-6-carbonitrile

A mixture of 2-((2-fluoro-4-methylbenzyl)oxy)-6-(piperidin-4-yl)pyridine (57 mg, crude), (S)-2-(chloromethyl)-3-(oxetan-2-ylmethyl)-3H-imidazo[4,5-c]pyridine-6-carbonitrile (50 mg, 0.19 mmol) and DIPEA (123 mg, 0.95 mmol) in DMF (6 mL) was stirred at 60° C. for 2 h. The mixture was filtered, and the filtrate was purified by prep-TLC (PE:EA=1:3) to obtain (S)-2-((4-(6-((2-fluoro-4-methylbenzyl)oxy)pyridin-2-yl)piperidin-1-yl)methyl)-3-(oxetan-2-ylmethyl)-3H-imidazo[4,5-c]pyridine-6-carbonitrile (25 mg, yield: 25%) as a white solid. MS Calcd: 526.2; MS Found: 527.3 [M+H]⁺.

### Step G: 2-[(2-fluoro-4-methylphenyl)methoxy]-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 496a)

2-[(2-fluoro-4-methylphenyl)methoxy]-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 496a) (5.0 mg) was obtained as a white solid.

MS Calcd: 636.3; MS Found: 637.3 [M+H]⁺.

¹H NMR (400 MHz, CD₃OD-d₄) 9.16 (s, 1H), 8.52 (s, 1H), 7.64 (t, J=7.8 Hz, 1H), 7.37 (t, J=7.8 Hz, 1H), 7.03-6.87 (m, 3H), 6.70 (d, J=8.0 Hz, 1H), 5.41 (s, 2H), 5.29-5.19 (m, 1H), 4.85-4.83 (m, 2H), 4.78-4.62 (m, 3H), 4.51-4.42 (m, 1H), 4.03-3.90 (m, 2H), 3.52-3.38 (m, 2H), 3.14-3.01 (m, 1H), 2.90-2.78 (m, 1H), 2.58-2.46 (m, 1H), 3.34 (s, 3H), 2.35-2.17 (m, 4H). ¹⁹F NMR (377 MHz, DMSO-d₆): δ −66.73, −76.98, −121.66.

### Example 302: 2-{[2-fluoro-4-(trifluoromethyl)phenyl]methoxy}-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 497a)

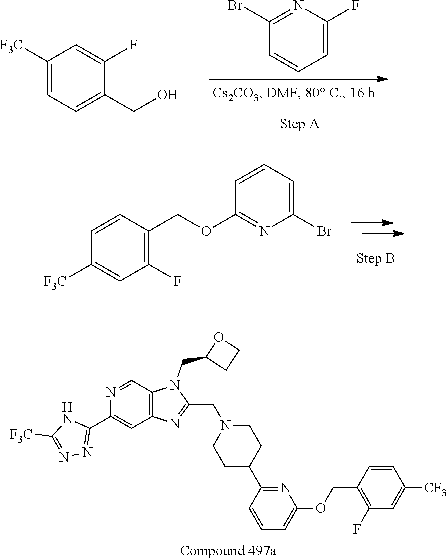 [SMILES: OCc1ccc(C(F)(F)F)cc1F ; Fc1cccc(Br)n1 ; Fc1cc(C(F)(F)F)ccc1COc1cccc(Br)n1 ; Fc1cc(C(F)(F)F)ccc1COc1cccc(C2CCN(Cc3nc4cc(-c5nnc(C(F)(F)F)[nH]5)ncc4n3C[C@@H]3CCO3)CC2)n1]

### Step A: 2-bromo-6-((2-fluoro-4-(trifluoromethyl)benzyl)oxy)pyridine

A mixture of (2-fluoro-4-(trifluoromethyl)phenyl)methanol (1000 mg, 5.15 mmol), 2-bromo-6-fluoropyridine (1082 mg, 6.18 mmol) and Cs₂CO₃ (5041 mg, 15.46 mmol) in DMF (75 mL) was stirred at 80° C. for 16 h. The mixture was poured into water (200 mL) and extracted with EtOAc (2×200 mL), the combined organic layer was washed with brine, dried over sodium sulfate, filtered and the residue was concentrated. The residue was purified by column chromatography on silica gel to obtain 2-bromo-6-((2-fluoro-4-(trifluoromethyl)benzyl)oxy)pyridine (800 mg, yield: 44%) as a yellow solid. MS Calcd: 349.0; MS Found: 349.9 [M+H]⁺.

### Step B: 2-{[2-fluoro-4-(trifluoromethyl)phenyl]methoxy}-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 497a)

2-{[2-fluoro-4-(trifluoromethyl)phenyl]methoxy}-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 497a) (11 mg) was obtained as a white solid.

MS Calcd: 690.2; MS Found: 691.3 [M+H]⁺.

¹H NMR (400 MHz, CD₃OD) 9.13 (s, 1H), 8.49 (s, 1H), 7.73-7.67 (m, 1H), 7.62-7.56 (m, 1H), 7.50-7.43 (m, 2H), 6.84 (dd, J₁=12.4 Hz/J₂=5.2 Hz, 1H), 6.68 (dd, J₁=12.8 Hz/J₂=8.0 Hz, 1H), 5.53 (s, 2H), 5.36-5.27 (m, 1H), 5.01-4.92 (m, 1H), 4.85-4.76 (m, 1H), 4.70-4.47 (m, 2H), 4.09-3.88 (m, 2H), 3.10-3.03 (m, 1H), 2.96-2.79 (m, 2H), 2.69-2.50 (m, 2H), 2.40-2.23 (m, 2H), 1.91-1.77 (m, 4H). ¹⁹F NMR (377 MHz, CD₃OD): δ −64.17, −66.64, −118.03.

### Example 303: 2-[(4-cyclopropyl-2-fluorophenyl)methoxy]-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 498a)

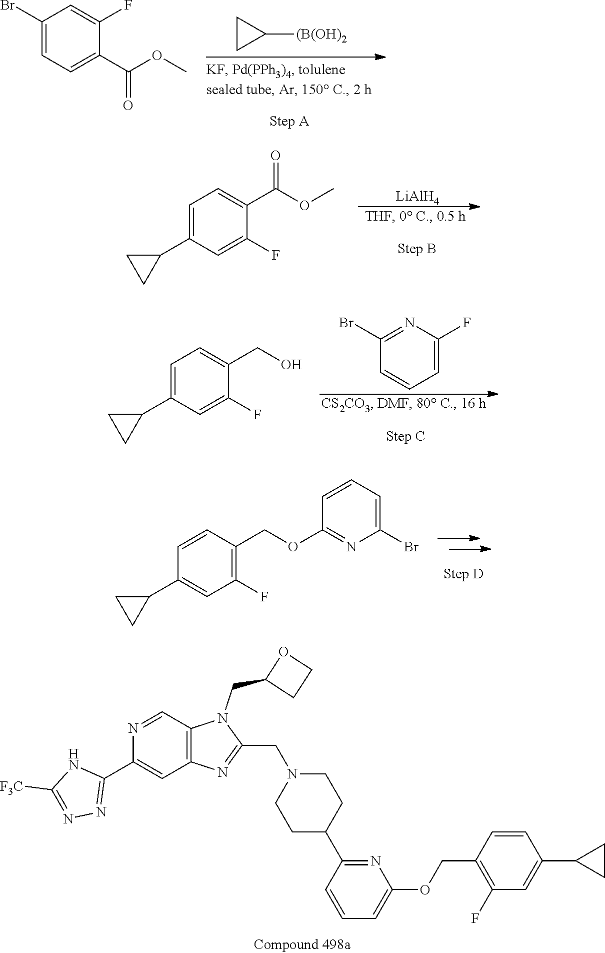

### Step A: methyl 4-cyclopropyl-2-fluorobenzoate

A mixture of methyl 4-bromo-2-fluorobenzoate (2.0 g, 8.6 mmol), cyclopropylboronic acid (2.2 g, 25.7 mmol), Pd(PPh₃)₄ (496 mg, 0.43 mmol) and KF (1.5 g, 25.8 mmol) in toluene (10 mL) was stirred at 150° C. under Ar in a sealed tube for 2 h. The mixture was poured into water (200 mL) and extracted with EtOAc (2×200 mL), the combined organic layer was washed with brine, dried over sodium sulfate, filtered and the residue was concentrated. The residue was purified by column chromatography on silica gel to obtain methyl 4-cyclopropyl-2-fluorobenzoate (1.2 g, yield: 72%) as a clear oil. MS Calcd: 194.1; MS Found: 195.1 [M+H]⁺.

### Step B: (4-cyclopropyl-2-fluorophenyl)methanol

To a solution of LiAlH₄ (141 mg, 3.71 mmol) in THF (5 mL) were added methyl 4-cyclopropyl-2-fluorobenzoate (600 mg, 3.1 mmol) in THF (5 mL) dropwisely at 0° C. under Ar. The mixture was stirred at 0° C. for 0.5 h. The mixture was added dropwise water (0.5 mL) and DCM (45 mL). 50 mL of water was added to the mixture and the organic layer was separated, dried over sodium sulfate, filtered and the residue was concentrated to obtain (4-cyclopropyl-2-fluorophenyl)methanol (520 mg, crude) as a yellow oil.

### Step C: 2-bromo-6-((4-cyclopropyl-2-fluorobenzyl)oxy)pyridine

A mixture of (4-cyclopropyl-2-fluorophenyl)methanol (520 mg), 2-bromo-6-fluoropyridine (551 mg, 3.1 mmol) and Cs₂CO₃ (3.1 g, 9.4 mmol) in DMF (5 mL) was stirred at 80° C. for 16 h. The mixture was poured into water (100 mL) and extracted with EtOAc (2×100 mL), the combined organic layer was washed with brine, dried over sodium sulfate, filtered and the residue was concentrated. The residue was purified by column chromatography on silica gel to obtain 2-bromo-6-((4-cyclopropyl-2-fluorobenzyl)oxy)pyridine (920 mg, yield: 91%) as a yellow solid. MS Calcd: 321.0; MS Found: 322.0 [M+H]⁺.

### Step D: 2-[(4-cyclopropyl-2-fluorophenyl)methoxy]-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 498a)

2-[(4-cyclopropyl-2-fluorophenyl)methoxy]-6-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyridine (Compound 498a) (12 mg) was obtained as a white solid.

MS Calcd: 662.3; MS Found: 663.3 [M+H]⁺.

¹H NMR (400 MHz, DMSO-d₆) 9.13 (s, 1H), 8.35 (s, 1H), 7.61 (t, J=7.2 Hz, 1H), 7.39 (t, J=6.0 Hz, 1H), 6.95-6.82 (m, 3H), 6.64 (d, J=8.0 Hz, 1H), 5.33 (s, 2H), 5.25-5.15 (m, 1H), 4.98-4.88 (m, 1H), 4.80-4.72 (m, 1H), 4.52-4.40 (m, 2H), 4.00 (d, J=13.2 Hz, 1H), 3.85 (d, J=14.0 Hz, 1H), 3.10-3.00 (m, 1H), 2.91-2.84 (m, 1H), 2.80-2.70 (m, 1H), 2.70-2.55 (m, 1H), 2.35-2.18 (m, 3H), 1.98-1.88 (m, 1H), 1.87-1.60 (m, 4H), 0.98-0.90 (m, 2H), 0.70-0.60 (m, 2H). ¹⁹F NMR (377 MHz, DMSO-d₆): −63.07, −119.10, −119.22.

### Example 304: 2-[(4-chloro-2-fluorophenyl)methoxy]-6-[(3,4-trans)-3-fluoro-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl]pyridine (Compound 499a)

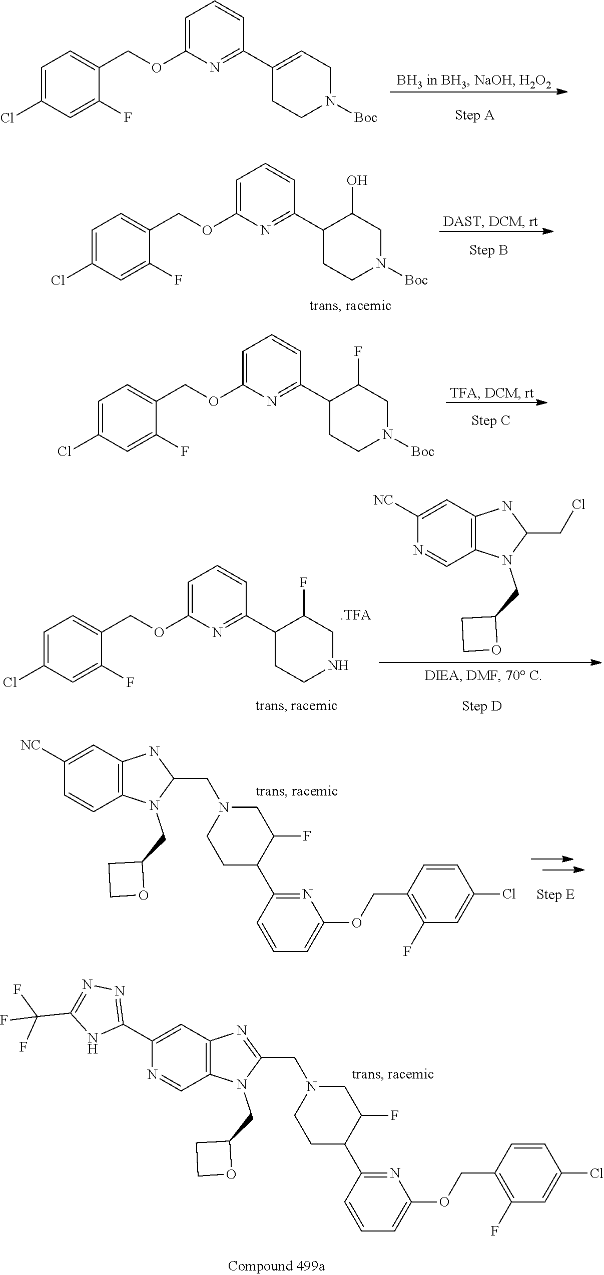

### Step A: tert-butyl (3,4-trans)-4-{6-[(4-chloro-2-fluorophenyl)methoxy]pyridin-2-yl}-3-hydroxypiperidine-1-carboxylate

To a solution of tert-butyl 6-((4-chloro-2-fluorobenzyl)oxy)-3′,6′-dihydro-[2,4′-bipyridine]-1′(2′H)-carboxylate (2.3 g, 5.5 mmol) in THE (40 mL) was added BH₃ (11 mL, 11.0 mmol, 1 M in THF) at room temperature, and the mixture was stirred for 4 h. Then 2 M NaOH aqueous solution (8.3 mL, 16.5 mmol) was added, the mixture was stirred at room temperature for 10 mins, Hydrogen peroxide (8.3 mL, 30% aqueous solution) was added, the mixture was heated to 50° C. and stirred for 1.5 h. The resulting mixture diluted with EA (100 mL) and washed with water (50 mL*2). The organic layer was dried over Na₂SO₄, filtered and concentrated. The residue was purified by column chromatography on silica gel (PE/EA=4/1) to give tert-butyl (3,4-trans)-4-{6-[(4-chloro-2-fluorophenyl)methoxy]pyridin-2-yl}-3-hydroxypiperidine-1-carboxylate (1.2 g, 52% yield) as colorless oil. MS Calcd.: 436.2; MS Found: 437.3 [M+H]⁺.

¹H NMR (400 MHz, DMSO-d₆): δ 7.57 (dd, J=8.4 Hz, 7.2 Hz, 1H), 7.48 (t, J=8.0 Hz, 1H), 7.15-7.21 (m, 2H), 6.84 (d, J=7.2 Hz, 1H), 6.66 (dd, J=8.0 Hz, 0.4 Hz, 1H), 5.33-5.48 (m, 2H), 4.22-4.32 (m, 1H), 4.04-4.11 (m, 1H), 3.86 (dt, J=10.0 Hz, 4.8 Hz, 1H), 2.52-2.89 (m, 3H), 1.65-1.80 (m, 2H), 1.50 (s, 9H).

### Step B: tert-butyl (3S,4S)-4-{6-[(4-chloro-2-fluorophenyl)methoxy]pyridin-2-yl}-3-fluoropiperidine-1-carboxylate

To a solution of tert-butyl (3,4-trans)-4-{6-[(4-chloro-2-fluorophenyl)methoxy]pyridin-2-yl}-3-hydroxypiperidine-1-carboxylate (1.1 g, 2.5 mmol) in DCM (33 mL) was added DAST (609 mg, 3.8 mmol) at 0° C. and stirred for 1 h at rt. The mixture was diluted with EA (100 mL) and wished with water (50 mL×2). The organic layer was dried over Na₂SO₄, filtered and evaporated to dryness. The residue was purified by column chromatography on silica gel (PE:EA=20:1) to give tert-butyl (3,4-trans)-4-{6-[(4-chloro-2-fluorophenyl)methoxy]pyridin-2-yl}-3-fluoropiperidine-1-carboxylate (878 mg, 80% yield) as colorless oil. MS Calcd.: 438.2; MS Found: 439.0 [M+H]⁺.

¹H NMR (400 MHz, DMSO-d₆): δ 7.53 (dd, J=8.0 Hz, 7.2 Hz, 1H), 7.42 (t, J=8.4 Hz, 1H), 7.09-7.15 (m, 2H), 6.80 (d, J=7.2 Hz, 1H), 6.67 (dd, J=8.4 Hz, 1H), 5.35-5.46 (m, 2H), 4.88 (dt, J=10.0 Hz, 5.2 Hz, 0.5H), 4.76 (dt, J=10.0 Hz, 5.2 Hz, 0.5H), 4.33-4.58 (m, 1H), 3.98-4.28 (m, 1H), 2.77-2.95 (m, 3H), 1.80-1.90 (m, 2H), 1.50 (s, 9H).

### Step C: 2-[(4-chloro-2-fluorophenyl)methoxy]-6-[(3,4-trans)-3-fluoropiperidin-4-yl]pyridine

A mixture of tert-butyl (3S,4S)-4-{6-[(4-chloro-2-fluorophenyl)methoxy]pyridin-2-yl}-3-fluoropiperidine-1-carboxylate (142 mg, 0.32 mmol), TFA (0.5 mL) in DCM (2 mL) was stirred at room temperature for 2 h. After the reaction was completed, the mixture was concentrated in vacuum to give 2-[(4-chloro-2-fluorophenyl)methoxy]-6-[(3,4-trans)-3-fluoropiperidin-4-yl]pyridine. TFA salt (crude 210 mg) as yellow oil. MS Calcd.: 338.1; MS Found: 339.0 [M+H]⁺.

### Step D: 2-{[(3,4-trans)-4-{6-[(4-chloro-2-fluorophenyl)methoxy]pyridin-2-yl}-3-fluoropiperidin-1-yl]methyl}-3-{[(2S)-oxetan-2-yl]methyl}-3H-imidazo[4,5-c]pyridine-6-carbonitrile

A mixture of 2-[(4-chloro-2-fluorophenyl)methoxy]-6-[(3,4-trans)-3-fluoropiperidin-4-yl]pyridine. TFA salt (210 mg, crude), (S)-2-(chloromethyl)-3-(oxetan-2-ylmethyl)-3H-imidazo[4,5-c]pyridine-6-carbonitrile (80 mg, 0.30 mmol), DIEA (374 mg, 2.9 mmol) in DMF (3 mL) was stirred at 70° C. for 5 h. After the reaction was completed, the mixture was concentrated and the residue was purified by column chromatography on silica gel (DCM/MeOH=100/1) to give 2-{[(3,4-trans)-4-{6-[(4-chloro-2-fluorophenyl)methoxy]pyridin-2-yl}-3-fluoropiperidin-1-yl]methyl}-3-{[(2S)-oxetan-2-yl]methyl}-3H-imidazo[4,5-c]pyridine-6-carbonitrile (133 mg, 81% yield) as a yellow oil. MS Calcd.: 564.2; MS Found: 565.1 [M+H]⁺.

### Step E: 2-[(4-chloro-2-fluorophenyl)methoxy]-6-[(3,4-trans)-3-fluoro-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl]pyridine (Compound 499a)

2-[(4-chloro-2-fluorophenyl)methoxy]-6-[(3,4-trans)-3-fluoro-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl]pyridine (Compound 499a) (69 mg) as a white solid.

MS Calcd.: 674.2; MS Found: 675.4 [M+H]⁺.

¹H NMR (400 MHz, DMSO-d₆): δ 9.18 (dd, J=3.6 Hz, 0.8 Hz, 1H), 8.31 (dd, J=3.6 Hz, 0.8 Hz, 1H), 7.66 (t, J=7.6 Hz, 1H), 7.52-7.61 (m, 1H), 7.46 (dd, J=10.0 Hz, 2.4 Hz, 1H), 7.30 (dd, J=8.4 Hz, 2.0 Hz, 1H), 6.95 (d, J=7.2 Hz, 1H), 6.74 (d, J=8.0 Hz, 1H), 5.37-5.46 (m, 2H), 5.12-5.22 (m, 1H), 4.72-5.02 (m, 3H), 4.37-4.52 (m, 2H), 4.06-4.15 (m, 1H), 3.94-4.03 (m, 1H), 3.34-3.43 (m, 0.5H), 3.22-3.30 (m, 0.5H), 2.67-2.98 (m, 3H), 2.41-2.53 (m, 1H), 2.20-2.40 (m, 2H), 1.66-1.85 (m, 2H).

¹⁹F NMR (377 MHz, DMSO-d₆): δ −63.70, −115.26, −115.28, −181.80, −181.88.

### Example 305: 2-[(4-chloro-2-fluorophenyl)methoxy]-6-[(3,4-trans)-3-fluoro-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl]pyridine (Compound 500a)

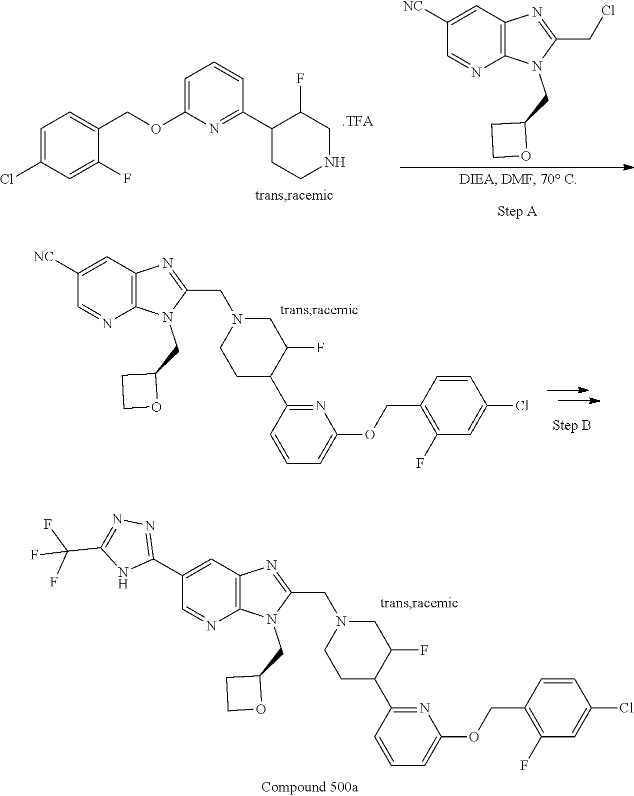 [SMILES: Fc1cc(Cl)ccc1COc1cccc(C2CCNCC2F)n1 ; N#Cc1cnc2c(c1)nc(CCl)n2C[C@@H]1CCO1 ; N#Cc1cnc2c(c1)nc(CN1CCC(c3cccc(OCc4ccc(Cl)cc4F)n3)C(F)C1)n2C[C@@H]1CCO1 ; Fc1cc(Cl)ccc1COc1cccc(C2CCN(Cc3nc4cc(-c5nnc(C(F)(F)F)[nH]5)cnc4n3C[C@@H]3CCO3)CC2F)n1]

### Step A: 2-{[(3,4-trans)-4-{6-[(4-chloro-2-fluorophenyl)methoxy]pyridin-2-yl}-3-fluoropiperidin-1-yl]methyl}-3-{[(2S)-oxetan-2-yl]methyl}-3H-imidazo[4,5-b]pyridine-6-carbonitrile

A mixture of 2-[(4-chloro-2-fluorophenyl)methoxy]-6-[(3,4-trans)-3-fluoropiperidin-4-yl]pyridine TFA salt (205 mg, crude), (S)-2-(chloromethyl)-3-(oxetan-2-ylmethyl)-3H-imidazo[4,5-b]pyridine-6-carbonitrile (80 mg, 0.31 mmol), DIEA (374 mg, 2.9 mmol) in DMF (3 mL) was stirred at 70° C. for 4 h. After the reaction was completed, the mixture was concentrated and the residue was purified by column chromatography on silica gel (DCM/MeOH=100/1) to give 2-{[(3,4-trans)-4-{6-[(4-chloro-2-fluorophenyl)methoxy]pyridin-2-yl}-3-fluoropiperidin-1-yl]methyl}-3-{[(2S)-oxetan-2-yl]methyl}-3H-imidazo[4,5-b]pyridine-6-carbonitrile (158 mg, 90% yield) as a yellow oil. MS Calcd.: 564.2; MS Found: 565.4 [M+H]⁺.

### Step B: 2-[(4-chloro-2-fluorophenyl)methoxy]-6-[(3,4-trans)-3-fluoro-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl]pyridine (Compound 500a)

2-[(4-chloro-2-fluorophenyl)methoxy]-6-[(3,4-trans)-3-fluoro-1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl]pyridine (Compound 500a) (38 mg) was obtained as a white solid.

MS Calcd.: 674.2; MS Found: 675.4 [M+H]⁺.

¹H NMR (400 MHz, DMSO-d₆): δ 9.07 (d, J=2.0 Hz, 1H), 8.70 (d, J=2.0 Hz, 1H), 7.71 (t, J=7.6 Hz, 1H), 7.60 (t, J=8.4 Hz, 1H), 7.47 (dd, J=10.0 Hz, 2.0 Hz, 1H), 7.31 (dd, J=8.4 Hz, 2.0 Hz, 1H), 7.01 (d, J=7.2 Hz, 1H), 6.78 (d, J=8.4 Hz, 1H), 5.41 (s, 2H), 5.10-5.37 (m, 2H), 4.78-4.88 (m, 1H), 4.54-4.75 (m, 3H), 4.47-4.52 (m, 1H), 4.29-4.38 (m, 1H), 3.68-3.86 (m, 1H), 3.33-3.41 (m, 1H), 2.89-3.20 (m, 3H), 2.66-2.78 (m, 1H), 2.36-2.50 (m, 1H), 1.93-2.13 (m, 2H). ¹⁹F NMR (377 MHz, DMSO-d₆): δ −63.74, −115.17.

### Example 306: 1-{6-[(4-chloro-2-fluorophenyl)methoxy]-3-fluoropyridin-2-yl}-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 501a)

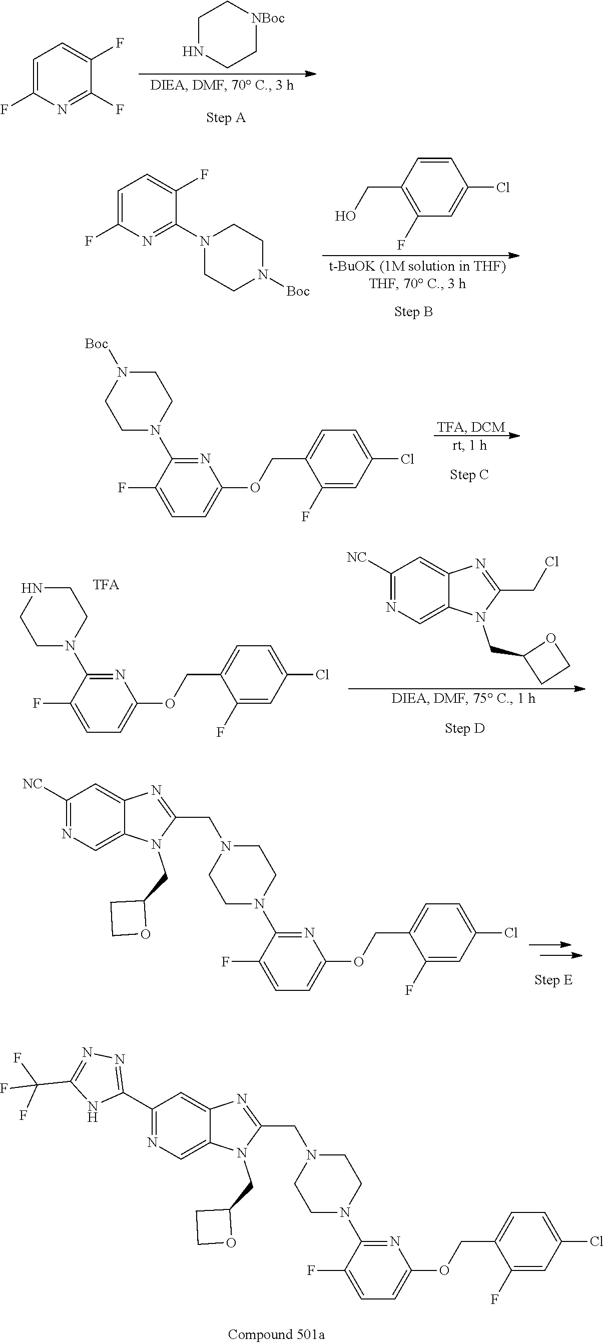

### Step A: tert-butyl 4-(3,6-difluoropyridin-2-yl)piperazine-1-carboxylate

To a solution of 2,3,6-trifluoropyridine (1.0 g, 7.5 mmol) in DMSO (15 mL) was added tert-butyl piperazine-1-carboxylate (1.7 g, 9.0 mmol) and DIEA (1.9 g, 15.0 mmol). The solution was stirred at 70° C. for 3 hours. The reaction mixture was cooled to room temperature, diluted with ethyl acetate (100 mL), washed with H₂O (50 mL×2). The organic layer was dried over anhydrous sodium sulfate, filtered and evaporated to dryness. The residue was purified by column chromatography on silica gel (PE/EA=5/1) to give to give tert-butyl 4-(3,6-difluoropyridin-2-yl)piperazine-1-carboxylate (2.2 g, yield: 98%) as a white solid. MS Calcd.: 299.1; MS Found: 200.1 [M-100+H]⁺.

### Step B: tert-butyl 4-(6-((4-chloro-2-fluorobenzyl)oxy)-3-fluoropyridin-2-yl)piperazine-1-carboxylate

To a solution of tert-butyl 4-(3,6-difluoropyridin-2-yl)piperazine-1-carboxylate (2.2 g, 7.4 mmol) in THF (15 mL) was added (4-chloro-2-fluorophenyl)methanol (1.3 g, 8.1 mmol) and t-BuOK (11 mL, 11.0 mmol, 1M solution in THF). The solution was stirred at 70° C. for 3 hours. The reaction mixture was cooled to room temperature, diluted with ethyl acetate (150 mL), washed with H₂O (100 mL×2). The organic layer was dried over anhydrous sodium sulfate, filtered and evaporated to dryness. The residue was purified by column chromatography on silica gel (PE/EA=15/1) to give to give tert-butyl 4-(6-((4-chloro-2-fluorobenzyl)oxy)-3-fluoropyridin-2-yl)piperazine-1-carboxylate (3.0 g, yield: 93%) as colorless oil. MS Calcd.: 439.2; MS Found: 384.1 [M-56+H]⁺.

### Step C: 1-(6-((4-chloro-2-fluorobenzyl)oxy)-3-fluoropyridin-2-yl)piperazine

To a solution of tert-butyl 4-(6-((4-chloro-2-fluorobenzyl)oxy)-3-fluoropyridin-2-yl)piperazine-1-carboxylate (1.0 g, 2.3 mmol) in DCM (10 mL) was added TFA (2 mL). The solution was stirred at room temperature for an hour. The solvent was removed in vacuo to give 1-(6-((4-chloro-2-fluorobenzyl)oxy)-3-fluoropyridin-2-yl)piperazine (1.2 g, TFA salt) as light-yellow oil. MS Calcd.: 339.1; MS Found: 340.1 [M+H]⁺.

### Step D: (S)-2-((4-(6-((4-chloro-2-fluorobenzyl)oxy)-3-fluoropyridin-2-yl)piperazin-1-yl)methyl)-3-(oxetan-2-ylmethyl)-3H-imidazo[4,5-c]pyridine-6-carbonitrile

To a solution of 1-(6-((4-chloro-2-fluorobenzyl)oxy)-3-fluoropyridin-2-yl)piperazine (133 mg, TFA salt) in DMF (3 mL) was added DIEA (103 mg, 0.80 mmol). After 2 minutes, (S)-2-(chloromethyl)-3-(oxetan-2-ylmethyl)-3H-imidazo[4,5-c]pyridine-6-carbonitrile (70 mg, 0.27 mmol) was added. The resulting mixture was heated to 75° C. for an hour. The reaction was quenched with water (15 mL), extracted with ethyl acetate (30 mL×2). The organic layers were combined, dried over Na₂SO₄, filtered and concentrated under vacuum. The residue was purified by column chromatography on silica gel (DCM:MeOH=30:1) to give (S)-2-((4-(6-((4-chloro-2-fluorobenzyl)oxy)-3-fluoropyridin-2-yl)piperazin-1-yl)methyl)-3-(oxetan-2-ylmethyl)-3H-imidazo[4,5-c]pyridine-6-carbonitrile (126 mg, 83% yield) as a brown solid. MS Calcd.: 565.2; MS Found: 566.2 [M+H]⁺.

### Step E: 1-{6-[(4-chloro-2-fluorophenyl)methoxy]-3-fluoropyridin-2-yl}-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 501a)

1-{6-[(4-chloro-2-fluorophenyl)methoxy]-3-fluoropyridin-2-yl}-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 501a) (15 mg) was obtained as a white solid.

MS Calcd.: 675.2; MS Found: 676.0 [M+H]⁺.

¹H NMR (400 MHz, DMSO-d₆): δ 9.23 (s, 1H), 8.34 (s, 1H), 7.43-7.55 (m, 3H), 7.31 (dd, J=8.0 Hz, 1.6 Hz, 1H), 6.30 (d, J=8.0 Hz, 1H), 5.30 (s, 2H), 5.12-5.19 (m, 1H), 4.89-4.98 (m, 1H), 4.73-4.81 (m, 1H), 4.48-4.56 (m, 1H), 4.15-4.47 (m, 3H), 3.20-3.52 (m, 4H), 2.80-3.15 (m, 4H), 2.70-2.80 (m, 1H), 2.35-2.53 (m, 1H). ¹⁹F NMR (377 MHz, DMSO-d₆): δ −63.73, −115.33, −140.48.

### Example 307: 1-{6-[(4-chloro-2-fluorophenyl)methoxy]-3-fluoropyridin-2-yl}-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 502a)

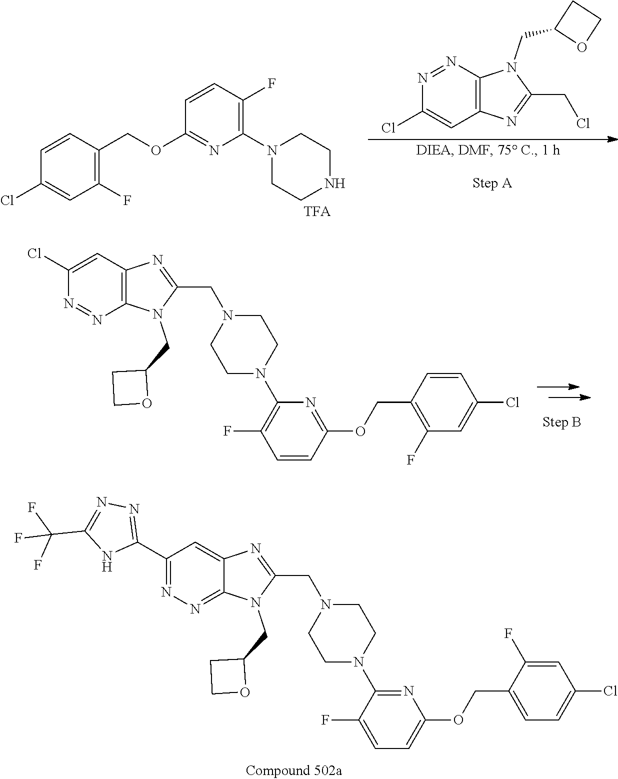 [SMILES: Fc1cc(Cl)ccc1COc1ccc(F)c(N2CCNCC2)n1 ; ClCc1nc2cc(Cl)nnc2n1C[C@@H]1CCO1 ; Fc1cc(Cl)ccc1COc1ccc(F)c(N2CCN(Cc3nc4cc(Cl)nnc4n3C[C@@H]3CCO3)CC2)n1 ; Fc1cc(Cl)ccc1COc1ccc(F)c(N2CCN(Cc3nc4cc(-c5nnc(C(F)(F)F)[nH]5)nnc4n3C[C@@H]3CCO3)CC2)n1]

### Step A: (S)-3-chloro-6-((4-(6-((4-chloro-2-fluorobenzyl)oxy)-3-fluoropyridin-2-yl)piperazin-1-yl)methyl)-7-(oxetan-2-ylmethyl)-7H-imidazo[4,5-c]pyridazine

To a solution of 1-(6-((4-chloro-2-fluorobenzyl)oxy)-3-fluoropyridin-2-yl)piperazine (550 mg, TFA salt) in DMF (8 mL) was added DIEA (427 mg, 3.3 mmol). After 2 minutes, (S)-3-chloro-6-(chloromethyl)-7-(oxetan-2-ylmethyl)-7H-imidazo[4,5-c]pyridazine (300 mg, 1.1 mmol) was added. The resulting mixture was stirred at 75° C. for an hour. After the reaction was completed, the mixture was diluted with EA (100 mL), washed with H₂O (50 mL×2). The organic layer was dried over anhydrous sodium sulfate, filtered and evaporated to dryness. The residue was purified by column chromatography on silica gel (DCM/MeOH=30/1) to give (S)-3-chloro-6-((4-(6-((4-chloro-2-fluorobenzyl)oxy)-3-fluoropyridin-2-yl)piperazin-1-yl)methyl)-7-(oxetan-2-ylmethyl)-7H-imidazo[4,5-c]pyridazine (470 mg, yield: 74%) as brown oil. MS Calcd.: 575.1; MS Found: 576.1 [M+H]⁺.

### Step B: 1-{6-[(4-chloro-2-fluorophenyl)methoxy]-3-fluoropyridin-2-yl}-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 502a)

1-{6-[(4-chloro-2-fluorophenyl)methoxy]-3-fluoropyridin-2-yl}-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 502a) (52 mg) was obtained as a white solid.

MS Calcd.: 676.2; MS Found: 677.0 [M+H]⁺.

¹H NMR (400 MHz, DMSO-d6): δ 8.51 (s, 1H), 7.42-7.55 (m, 3H), 7.30 (dd, J=8.4 Hz, 2.0 Hz, 1H), 6.25 (dd, J=8.4 Hz, 1.6 Hz, 1H), 5.25-5.32 (m, 3H), 5.00-5.08 (m, 1H), 4.87-4.94 (m, 1H), 4.49-4.56 (m, 1H), 4.39-4.45 (m, 1H), 4.02-4.12 (m, 2H), 3.40-3.48 (m, 4H), 2.70-2.80 (m, 1H), 2.60-2.70 (m, 4H), 2.48-2.59 (m, 1H). ¹⁹F NMR (377 MHz, DMSO-d₆): δ −63.48, −115.37, −140.55.

### Example 308: 3-fluoro-4-{[(5-fluoro-6-{4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazin-1-yl}pyridin-2-yl)oxy]methyl}benzonitrile (Compound 503a)

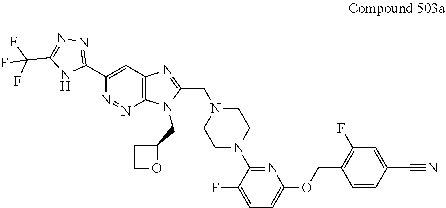 [SMILES: N#Cc1ccc(COc2ccc(F)c(N3CCN(Cc4nc5cc(-c6nnc(C(F)(F)F)[nH]6)nnc5n4C[C@@H]4CCO4)CC3)n2)c(F)c1]

3-fluoro-4-{[(5-fluoro-6-{4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazin-1-yl}pyridin-2-yl)oxy]methyl}benzonitrile (Compound 503a) (11 mg) was obtained as a white solid.

MS Calcd.: 667.2; MS Found: 668.0 [M+H]⁺.

¹H NMR (400 MHz, DMSO-d₆): δ 8.61 (s, 1H), 7.88 (dd, J=10.0 Hz, 1.2 Hz, 1H), 7.63-7.75 (m, 2H), 7.51-7.59 (m, 1H), 6.36 (d, J=8.8 Hz, 1H), 5.41 (s, 2H), 5.18-5.27 (m, 1H), 4.95-5.04 (m, 1H), 4.82-4.90 (m, 1H), 4.47-4.55 (m, 1H), 4.33-4.41 (m, 1H), 3.60-3.81 (m, 5H), 3.00-3.23 (m, 5H), 2.65-2.81 (m, 2H). ¹⁹F NMR (377 MHz, DMSO-d₆): δ −63.67, −115.60, −140.23.

### Example 309: 4-[(4-chloro-2-fluorophenyl)methoxy]-5-fluoro-2-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}pyrimidine (Compound 504a)

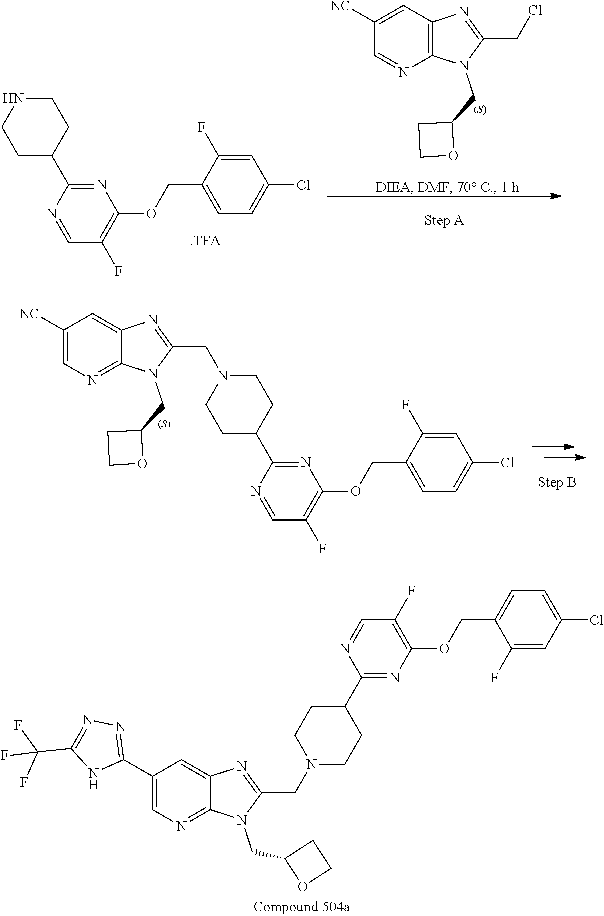

### Step A: (S)-2-((4-(4-((4-chloro-2-fluorobenzyl)oxy)-5-fluoropyrimidin-2-yl)piperidin-1-yl)methyl)-3-(oxetan-2-ylmethyl)-3H-imidazo[4,5-b]pyridine-6-carbonitrile

To a solution of 4-((4-chloro-2-fluorobenzyl)oxy)-5-fluoro-2-(piperidin-4-yl)pyrimidine TFA salt (307 mg) in DMF (3 mL) was added DIEA (491 mg, 3.8 mmol). After 2 minutes, (S)-2-(chloromethyl)-3-(oxetan-2-ylmethyl)-3H-imidazo[4,5-b]pyridine-6-carbonitrile (100 mg, 0.38 mmol) was added. The mixture was stirred at 70° C. for 1 hour. After the reaction was completed, the mixture was evaporated to dryness. The residue was purified by column chromatography on silica gel (DMC/MeOH=60/1) to give (S)-2-((4-(4-((4-chloro-2-fluorobenzyl)oxy)-5-fluoropyrimidin-2-yl)piperidin-1-yl)methyl)-3-(oxetan-2-ylmethyl)-3H-imidazo[4,5-b]pyridine-6-carbonitrile (200 mg, 93% yield) as a brown solid. MS Calcd.: 565.2; MS Found: 566.3 [M+H]⁺.

### Step B: 4-[(4-chloro-2-fluorophenyl)methoxy]-5-fluoro-2-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}pyrimidine (Compound 504a)

4-[(4-chloro-2-fluorophenyl)methoxy]-5-fluoro-2-{1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}pyrimidine (Compound 504a) (20 mg) was obtained.

MS Calcd.: 675.2; MS Found: 676.0 [M+H]⁺.

¹H NMR (400 MHz, DMSO-d₆): δ 8.99 (d, J=2.0 Hz, 1H), 8.54-8.58 (m, 2H), 7.62 (t, J=8.0 Hz, 1H), 7.51 (dd, J=10.0 Hz, J=2.0 Hz, 1H), 7.34 (dd, J=8.4 Hz, J=2.0 Hz, 1H), 5.55 (s, 2H), 5.15-5.25 (m, 1H), 4.81-4.90 (m, 1H), 4.70-4.76 (m, 1H), 4.45-4.53 (m, 1H), 4.35-4.42 (m, 1H), 3.90-4.02 (m, 2H), 2.88-3.00 (m, 2H), 2.65-2.82 (m, 3H), 2.21-2.35 (m, 2H), 1.88-1.98 (m, 2H), 1.70-1.87 (m, 2H). ¹⁹F NMR (377 MHz, DMSO-d₆): δ −63.21, −114.67, −157.68.

### Example 310: 2-[(4-chloro-2-fluorophenyl)methoxy]-4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyrimidine (Compound 505a)

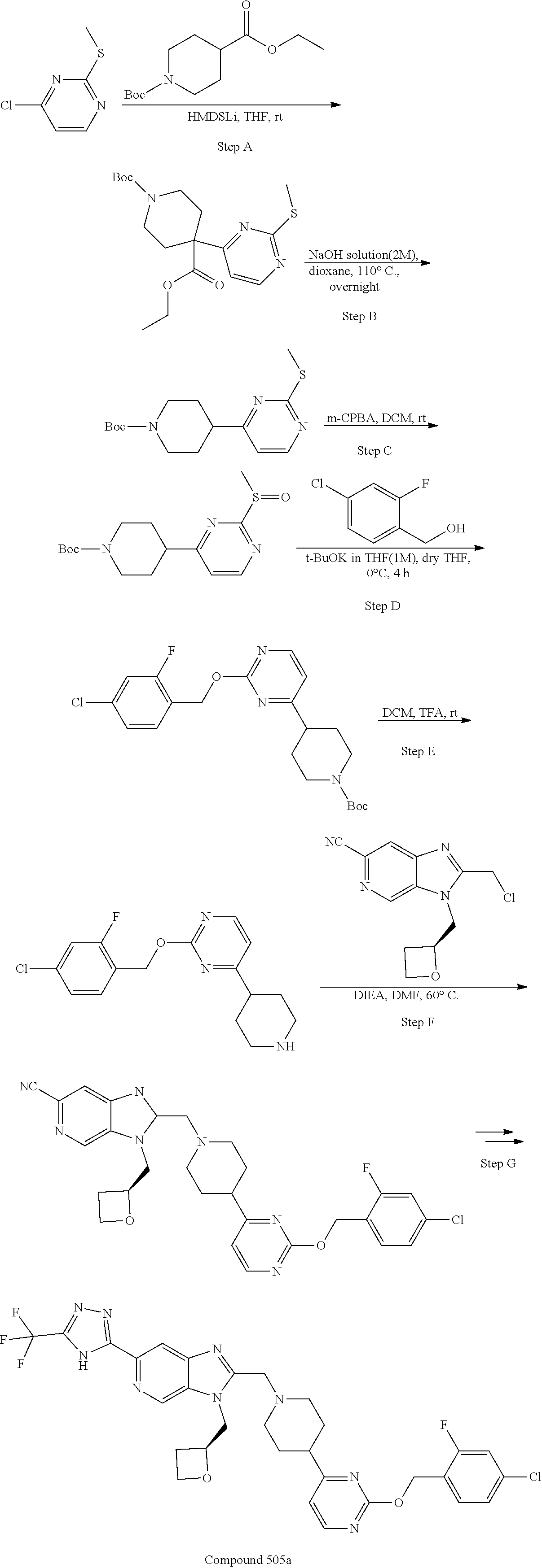

### Step A: 1-(tert-butyl) 4-ethyl 4-(2-(methylthio)pyrimidin-4-yl)piperidine-1,4-dicarboxylate

To a mixture of 4-chloro-2-(methylthio)pyrimidine (6.7 g, 41.9 mmol), 1-(tert-butyl) 4-ethyl piperidine-1,4-dicarboxylate (11.8 g, 45.9 mmol) in THF (50 mL) was added LiHMDS (1 M in THF, 50 mL, 50 mmol) at rt. The resulting mixture was stirred at room temperature overnight. After the reaction was completed, the mixture was diluted with EA (150 mL), washed with H₂O (120 mL*2). The organic layer was dried over anhydrous sodium sulfate, filtered and evaporated to dryness. The residue was purified by column chromatography on silica gel (PE/EA=6/1) to give 1-(tert-butyl) 4-ethyl 4-(2-(methylthio)pyrimidin-4-yl)piperidine-1,4-dicarboxylate (13.2 g, yield: 83%). MS Calcd.: 381.2; MS Found: 382.3 [M+H]⁺.

### Step B: tert-butyl 4-(2-(methylthio)pyrimidin-4-yl)piperidine-1-carboxylate

A mixture of 1-(tert-butyl) 4-ethyl 4-(2-(methylthio)pyrimidin-4-yl)piperidine-1,4-dicarboxylate (1.8 g, 4.72 mmol), NaOH solution (2 M aq., 10 mL, 20.0 mmol) in dioxane (10 mL) was stirred at 110° C. overnight. After the reaction was completed, the mixture was diluted with EA (20 mL), washed with H₂O (10 mL*2). The organic layer was dried over anhydrous sodium sulfate, filtered and evaporated to dryness. The residue was purified by column chromatography on silica gel (PE/EA=5/1) to give tert-butyl 4-(2-(methylthio)pyrimidin-4-yl)piperidine-1-carboxylate (1.1 g, yield: 76%) as a brown solid. MS Calcd.: 309.1; MS Found: 310.1 [M+H]⁺.

### Step C: tert-butyl 4-(2-(methylsulfinyl)pyrimidin-4-yl)piperidine-1-carboxylate

To a solution of tert-butyl 4-(2-(methylthio)pyrimidin-4-yl)piperidine-1-carboxylate (7.0 g, 22.6 mmol) in DCM (70 mL) was added m-CPBA (4.675 g, 27.2 mmol) at 0° C. The mixture was stirred at room temperature for 7 hours. After the reaction was completed, the mixture was filtered. The filtrate was washed with Sat. NaHSO₃ (50 mL×2) and brine (50 mL). The organic layer was dried over anhydrous sodium sulfate, filtered and evaporated to dryness. The residue was purified by column chromatography on silica gel (PE/EA=1/6) to give tert-butyl 4-(2-(methylsulfinyl)pyrimidin-4-yl)piperidine-1-carboxylate (2.5 g, yield: 34%). MS Calcd.: 325.2; MS Found: 326.1 [M+H]⁺.

### Step D: tert-butyl 4-(2-((4-chloro-2-fluorobenzyl)oxy)pyrimidin-4-yl)piperidine-1-carboxylate

To a solution of tert-butyl 4-(2-(methylsulfinyl)pyrimidin-4-yl)piperidine-1-carboxylate (1.25 g, 3.85 mmol), (4-chloro-2-fluorophenyl)methanol (616 mg, 3.8 mmol) in dry THF (10 mL) was added t-BuOK in THF (4.6 mL, 4.6 mmol) dropwisely at 0° C. The mixture was stirred at 0° C. for 4 h. After the reaction was completed, the mixture was diluted with EA (40 mL), washed with H₂O (40 mL). The organic layer was dried over anhydrous sodium sulfate, filtered and evaporated to dryness. The residue was purified by column chromatography on silica gel (PE/EA=1/1) to give tert-butyl 4-(2-((4-chloro-2-fluorobenzyl)oxy)pyrimidin-4-yl)piperidine-1-carboxylate (1.5 g, yield: 92%). MS Calcd.: 421.2; MS Found: 422.1 [M+H]⁺.

### Step E: 2-((4-chloro-2-fluorobenzyl)oxy)-4-(piperidin-4-yl)pyrimidine

To a solution of tert-butyl 4-(2-((4-chloro-2-fluorobenzyl)oxy)pyrimidin-4-yl)piperidine-1-carboxylate (79 mg, 0.19 mmol) in DCM (3 mL) was added TFA (1 mL) and the mixture stirred at room temperature for 1 hour. After the reaction was completed, the reaction was concentrated under vacuum to give 2-((4-chloro-2-fluorobenzyl)oxy)-4-(piperidin-4-yl)pyrimidine (90 mg, TFA salt, crude). MS Calcd.: 321.1; MS Found: 322.0 [M+H]⁺.

### Step F: (S)-2-((4-(2-((4-chloro-2-fluorobenzyl)oxy)pyrimidin-4-yl)piperidin-1-yl)methyl)-3-(oxetan-2-ylmethyl)-3H-imidazo[4,5-c]pyridine-6-carbonitrile

A mixture of 2-((4-chloro-2-fluorobenzyl)oxy)-4-(piperidin-4-yl)pyrimidine (90 mg, crude) and (S)-2-(chloromethyl)-3-(oxetan-2-ylmethyl)-3H-imidazo[4,5-c]pyridine-6-carbonitrile (40 mg, 0.15 mmol), DIEA (1.0 mL, 5.6 mmol) in DMF (3 mL) was stirred at 60° C. overnight. After the reaction was completed, the mixture was evaporated to dryness. The residue was purified by column chromatography on silica gel (DCM/MeOH=80/1) to give (S)-2-((4-(2-((4-chloro-2-fluorobenzyl)oxy)pyrimidin-4-yl)piperidin-1-yl)methyl)-3-(oxetan-2-ylmethyl)-3H-imidazo[4,5-c]pyridine-6-carbonitrile (64 mg, yield: 77%). MS Calcd.: 547.2; MS Found: 548.0 [M+H]⁺.

### Step G: 2-[(4-chloro-2-fluorophenyl)methoxy]-4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyrimidine (Compound 505a)

2-[(4-chloro-2-fluorophenyl)methoxy]-4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyrimidine (Compound 505a) (27 mg) was obtained.

MS Calcd.: 657.2; MS Found: 658.2 [M+H]⁺.

¹H NMR (400 MHz, DMSO-d₆): δ 9.16 (d, J=0.4 Hz, 1H), 8.51 (d, J=5.2 Hz, 1H), 8.28 (d, J=0.8 Hz, 1H), 7.58 (t, J=8.0 Hz, 1H), 7.49 (dd, J=10.0 Hz, J=2.0 Hz, 1H), 7.32 (dd, J=8.4 Hz, J=2.0 Hz, 1H), 7.10 (d, J=5.2 Hz, 1H), 5.40 (s, 2H), 5.11-5.20 (m, 1H), 4.89-4.98 (m, 1H), 4.74-4.79 (m, 1H), 4.47-4.55 (m, 1H), 4.38-4.46 (m, 1H), 4.01 (d, J=13.6 Hz, 1H), 3.86 (d, J=13.6 Hz, 1H), 2.98-3.04 (m, 1H), 2.86-2.91 (m, 1H), 2.60-2.80 (m, 2H), 2.40-2.53 (m, 1H), 2.16-2.32 (m, 2H), 1.60-1.90 (m, 4H). ¹⁹F NMR (377 MHz, DMSO-d₆): δ −63.69, −115.10.

### Example 311: 3-fluoro-4-({2-fluoro-5-[(3S)-3-methyl-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazin-1-yl]phenoxy}methyl)benzonitrile (Compound 506a)

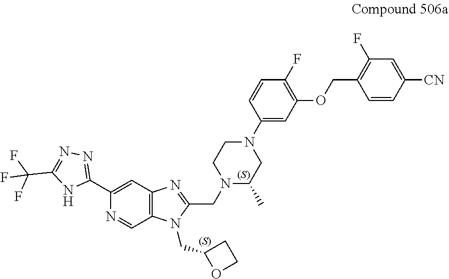

3-fluoro-4-({2-fluoro-5-[(3S)-3-methyl-4-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazin-1-yl]phenoxy}methyl)benzonitrile (Compound 506a) (13 mg) was obtained as a white solid.

MS Calcd.: 679.2; MS Found: 680.1 [M+H]⁺.

¹H NMR (400 MHz, DMSO-d₆): δ 9.27 (s, 1H), 8.36 (s, 1H), 7.93 (d, J=10.4 Hz, 1H), 7.74-7.80 (m, 2H), 7.11 (dd, J=10.8 Hz, J=8.8 Hz, 1H), 6.92 (dd, J=7.2 Hz, J=2.4 Hz, 1H), 6.51-6.59 (m, 1H), 5.31 (s, 2H), 5.11-5.19 (m, 1H), 4.86-4.97 (m, 2H), 4.76-4.84 (m, 1H), 4.48-4.56 (m, 1H), 4.36-4.44 (m, 1H), 3.39-3.53 (m, 5H), 2.89-3.18 (m, 3H), 2.70-2.81 (m, 1H), 2.36-2.45 (m, 1H), 1.37 (d, J=4.8 Hz, 3H). ¹⁹F NMR (377 MHz, DMSO-d6): δ −63.71, −115.15, −145.29.

### Example 312: 3-fluoro-4-{[(4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyrimidin-2-yl)oxy]methyl}benzonitrile_(Compound 507a)

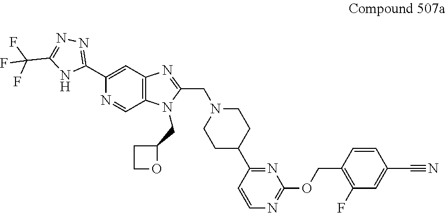 [SMILES: N#Cc1ccc(COc2nccc(C3CCN(Cc4nc5cc(-c6nnc(C(F)(F)F)[nH]6)ncc5n4C[C@@H]4CCO4)CC3)n2)c(F)c1]

3-fluoro-4-{[(4-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyrimidin-2-yl)oxy]methyl}benzonitrile (Compound 507a) (3 mg) was obtained as a white solid.

MS Calcd.: 648.2; MS Found: 649.1 [M+H].

¹H NMR (400 MHz, DMSO-d₆): δ 9.16 (s, 1H), 8.51 (d, J=9.2 Hz, 1H), 8.28 (s, 1H), 7.91 (d, J=10.0 Hz, 1H), 7.69-7.74 (m, 2H), 7.11 (d, J=5.2 Hz, 1H), 5.50 (s, 2H), 5.12-5.20 (m, 1H), 4.88-4.97 (m, 1H), 4.72-4.80 (m, 1H), 4.47-4.55 (m, 1H), 4.38-4.45 (m, 1H), 4.00 (d, J=13.6 Hz, 1H), 3.86 (d, J=13.6 Hz, 1H), 2.97-3.04 (m, 1H), 2.84-2.91 (m, 1H), 2.60-2.80 (m, 2H), 2.41-2.52 (m, 1H), 2.17-2.34 (m, 2H), 1.60-1.90 (m, 4H). ¹⁹F NMR (377 MHz, DMSO-d₆): δ −63.60, −115.36.

### Example 313: 2-[(4-chloro-2-fluorophenyl)methoxy]-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyrimidine (Compound 508a)

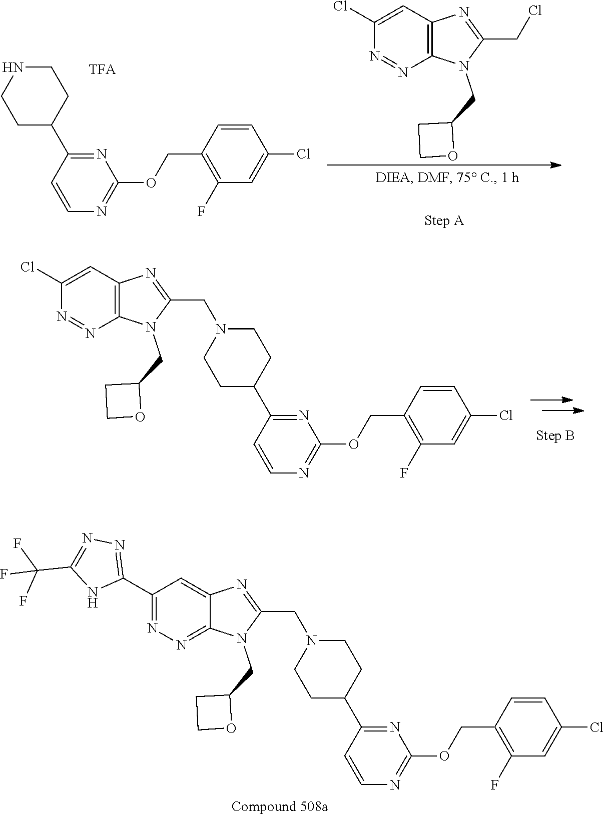 [SMILES: Fc1cc(Cl)ccc1COc1nccc(C2CCNCC2)n1 ; ClCc1nc2cc(Cl)nnc2n1C[C@@H]1CCO1 ; Fc1cc(Cl)ccc1COc1nccc(C2CCN(Cc3nc4cc(Cl)nnc4n3C[C@@H]3CCO3)CC2)n1 ; Fc1cc(Cl)ccc1COc1nccc(C2CCN(Cc3nc4cc(-c5nnc(C(F)(F)F)[nH]5)nnc4n3C[C@@H]3CCO3)CC2)n1]

### Step A: (S)-3-chloro-6-((4-(2-((4-chloro-2-fluorobenzyl)oxy)pyrimidin-4-yl)piperidin-1-yl)methyl)-7-(oxetan-2-ylmethyl)-7H-imidazo[4,5-c]pyridazine

A solution of 2-((4-chloro-2-fluorobenzyl)oxy)-4-(piperidin-4-yl)pyrimidine TFA salt (800 mg, crude), (S)-3-chloro-6-(chloromethyl)-7-(oxetan-2-ylmethyl)-7H-imidazo[4,5-c]pyridazine (484 mg, 1.8 mmol), DIEA (689 mg, 5.3 mmol) in DMF (5 mL) was stirred at 75° C. for 1 hour. After the reaction was completed, the mixture was diluted with EA (50 mL), washed with H₂O (40 mL×3). The combined organic layer was dried over anhydrous sodium sulfate, filtered and concentrated. The residue was purified by column chromatography on silica gel (DCM/MeOH=10/1) to give (S)-3-chloro-6-((4-(2-((4-chloro-2-fluorobenzyl)oxy)pyrimidin-4-yl)piperidin-1-yl)methyl)-7-(oxetan-2-ylmethyl)-7H-imidazo[4,5-c]pyridazine (800 mg, 81% yield, two steps) as yellow oil. MS Calcd.: 557.2; MS Found: 558.1[M+H]⁺.

### Step B: 2-[(4-chloro-2-fluorophenyl)methoxy]-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyrimidine (Compound 508a)

2-[(4-chloro-2-fluorophenyl)methoxy]-4-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyrimidine (Compound 508a) (13 mg) was obtained as a light-yellow solid.

MS Calcd.: 658.2; MS Found: 659.3 [M+H]⁺.

¹H NMR (400 MHz, CDCl₃): δ 8.54 (s, 1H), 8.50 (d, J=5.2 Hz, 1H), 7.48 (t, J=8.0 Hz, 1H), 7.12 (t, J=8.8 Hz, 2H), 6.90 (d, J=5.2 Hz, 1H), 5.38-5.50 (m, 3H), 4.97-5.14 (m, 2H), 4.86-4.96 (m, 1H), 4.72-4.80 (m, 1H), 4.52-4.68 (m, 2H), 3.87-3.98 (m, 2H), 3.25-3.45 (m, 2H), 2.87-3.02 (m, 2H), 2.55-2.64 (m, 1H), 2.20-2.40 (m, 4H). ¹⁹F NMR (377 MHz, CDCl₃): δ −65.22, −115.27.

### Example 314: 3-fluoro-4-{[(5-fluoro-2-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyrimidin-4-yl)oxy]methyl}benzonitrile (Compound 509a)

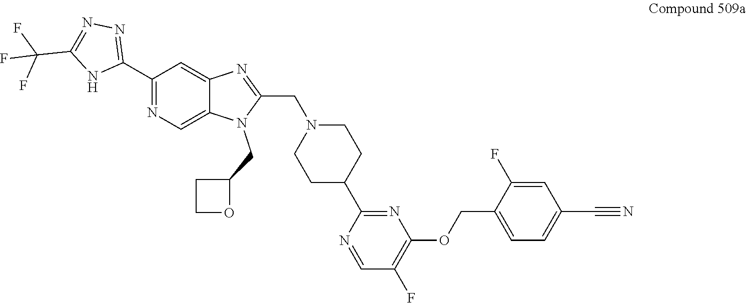 [SMILES: N#Cc1ccc(COc2nc(C3CCN(Cc4nc5cc(-c6nnc(C(F)(F)F)[nH]6)ncc5n4C[C@@H]4CCO4)CC3)ncc2F)c(F)c1]

3-fluoro-4-{[(5-fluoro-2-{1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}pyrimidin-4-yl)oxy]methyl}benzonitrile (Compound 509a) (1.6 mg) was obtained as a white solid.

MS Calcd.: 666.2; MS Found: 667.5 [M+H]⁺.

¹H NMR (400 MHz, DMSO-d₆): δ 8.59 (d, J=3.2 Hz, 1H), 8.35-8.50 (m, 1H), 8.18-8.25 (m, 1H), 7.93 (d, J=9.6 Hz, 1H), 7.73-7.80 (m, 2H), 5.64 (s, 2H), 5.10-5.23 (m, 1H), 4.80-4.97 (m, 1H), 4.68-4.81 (m, 1H), 4.45-4.57 (m, 1H), 4.32-4.43 (m, 1H), 3.93-4.02 (m, 1H), 3.81-3.91 (m, 1H), 2.96-3.03 (m, 1H), 2.82-2.91 (m, 1H), 2.65-2.80 (m, 2H), 2.20-2.35 (m, 1H), 1.85-1.97 (m, 2H), 1.69-1.77 (m, 2H), 1.30-1.40 (m, 2H). ¹⁹F NMR (377 MHz, DMSO-d₆): δ −61.60, −114.98, −157.57.

### Example 315: 4-[(4-chloro-2-fluorophenyl)methoxy]-5-fluoro-2-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyrimidine (Compound 510a)

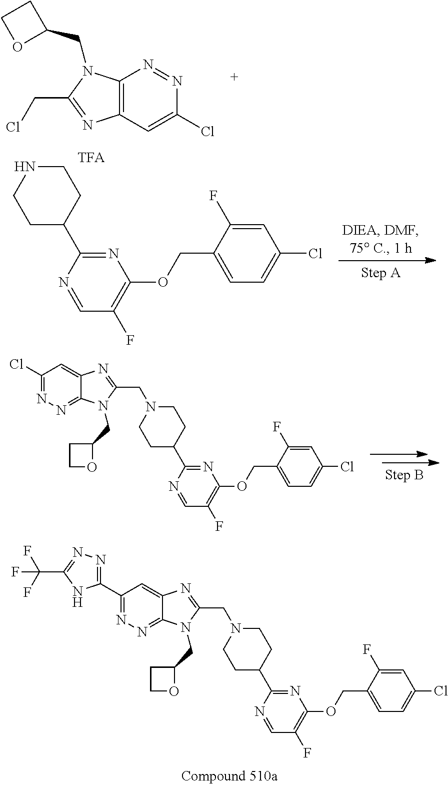 [SMILES: ClCc1nc2cc(Cl)nnc2n1C[C@@H]1CCO1 ; Fc1cc(Cl)ccc1COc1nc(C2CCNCC2)ncc1F ; Fc1cc(Cl)ccc1COc1nc(C2CCN(Cc3nc4cc(Cl)nnc4n3C[C@@H]3CCO3)CC2)ncc1F ; Fc1cc(Cl)ccc1COc1nc(C2CCN(Cc3nc4cc(-c5nnc(C(F)(F)F)[nH]5)nnc4n3C[C@@H]3CCO3)CC2)ncc1F]

### Step A: (S)-3-chloro-6-((4-(4-((4-chloro-2-fluorobenzyl)oxy)-5-fluoropyrimidin-2-yl)piperidin-1-yl)methyl)-7-(oxetan-2-ylmethyl)-7H-imidazo[4,5-c]pyridazine

To a solution of 4-((4-chloro-2-fluorobenzyl)oxy)-5-fluoro-2-(piperidin-4-yl)pyrimidine TFA salt (733 mg, crude) in DMF (10 mL) was added DIEA (569 mg, 4.4 mmol). After 2 minutes, (S)-3-chloro-6-(chloromethyl)-7-(oxetan-2-ylmethyl)-7H-imidazo[4,5-c]pyridazine (400 mg, 1.5 mmol) was added. The resulting mixture was heated to 75° C. for an hour. The reaction was quenched with water (10 mL), extracted with ethyl acetate (20 mL×3). The organic layers were combined, dried over Na₂SO₄, filtered and concentrated under vacuum. The residue was purified by column chromatography on silica gel (DCM:MeOH=30:1) to give (S)-3-chloro-6-((4-(4-((4-chloro-2-fluorobenzyl)oxy)-5-fluoropyrimidin-2-yl)piperidin-1-yl)methyl)-7-(oxetan-2-ylmethyl)-7H-imidazo[4,5-c]pyridazine (760 mg, 90% yield) as brown oil. MS Calcd.: 575.1; MS Found: 576.1[M+H]⁺.

### Step B: 4-[(4-chloro-2-fluorophenyl)methoxy]-5-fluoro-2-{1-[(7-{1[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyrimidine (Compound 510a)

4-[(4-chloro-2-fluorophenyl)methoxy]-5-fluoro-2-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyrimidine (Compound 510a) (15 mg) was obtained as a white solid.

MS Calcd.: 676.2; MS Found: 677.0 [M+H]⁺.

¹H NMR (400 MHz, DMSO-d₆): δ 8.56 (d, J=2.8 Hz, 1H), 8.51 (s, 1H), 7.62 (t, J=8.4 Hz, 1H), 7.51 (dd, J=10.0 Hz, 2.0 Hz, 1H), 7.34 (dd, J=8.4 Hz, 2.0 Hz, 1H), 5.55 (s, 2H), 5.24-5.33 (m, 1H), 5.00-5.09 (m, 1H), 4.87-4.96 (m, 1H), 4.47-4.55 (m, 1H), 4.38-4.47 (m, 1H), 4.00-4.10 (m, 2H), 2.92-3.02 (m, 2H), 2.70-2.83 (m, 2H), 2.53-2.60 (m, 1H), 2.25-2.38 (m, 2H), 1.89-1.99 (m, 2H), 1.72-1.87 (m, 2H). ¹⁹F NMR (377 MHz, DMSO-d₆): δ −63.58, −114.67, −157.65.

### Example 316: 4-[(4-chloro-2-fluorophenyl)methoxy]-5-fluoro-2-{4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazin-1-yl}pyrimidine (Compound 511a)

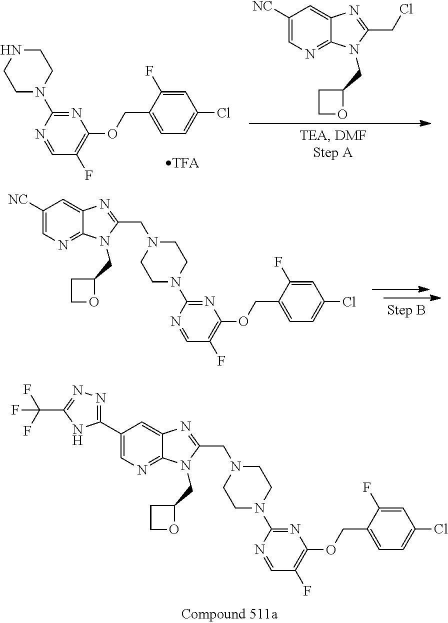

### Step A: (S)-2-((4-(4-((4-chloro-2-fluorobenzyl)oxy)-5-fluoropyrimidin-2-yl)piperazin-1-yl)methyl)-3-(oxetan-2-ylmethyl)-3H-imidazo[4,5-b]pyridine-6-carbonitrile

A mixture of 4-((4-chloro-2-fluorobenzyl)oxy)-5-fluoro-2-(piperazin-1-yl)pyrimidine TFA salt (967 mg, crude), (S)-2-(chloromethyl)-3-(oxetan-2-ylmethyl)-3H-imidazo[4,5-b]pyridine-6-carbonitrile (200 mg, 0.76 mmol), TEA (2 mL) in DMF (6 mL) was stirred at 50° C. for 2 hours. After the reaction was completed, the reaction was quenched with H₂O (20 mL) and extracted with ethyl acetate (25 mL×3). The organic layer was combined and washed with brine (20 mL×2), dried over sodium sulfate, filtered and concentrated in vacuum. The reaction was purified by column chromatography (DCM:MeOH=50:1) to give (S)-2-((4-(4-((4-chloro-2-fluorobenzyl)oxy)-5-fluoropyrimidin-2-yl)piperazin-1-yl)methyl)-3-(oxetan-2-ylmethyl)-3H-imidazo[4,5-b]pyridine-6-carbonitrile (462 mg, 80% yield) as yellow oil. MS Calcd.: 566.2; MS Found: 567.2 [M+H]⁺.

### Step B: 4-[(4-chloro-2-fluorophenyl)methoxy]-5-fluoro-2-{4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazin-1-yl}pyrimidine (Compound 511a)

4-[(4-chloro-2-fluorophenyl)methoxy]-5-fluoro-2-{4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazin-1-yl}pyrimidine (Compound 511a) (11 mg) was obtained as a white solid.

MS Calcd.: 676.2; MS Found: 677.2 [M+H]⁺.

¹H NMR (400 MHz, DMSO-d₆): δ 9.05 (s, 1H), 8.66 (s, 1H), 8.25 (d, J=2.8 Hz, 1H), 7.60 (t, J=8.0 Hz, 1H), 7.50 (dd, J=10 Hz, J=1.6 Hz, 1H), 7.34 (dd, J=8.4 Hz, J=2.0 Hz, 1H), 5.49 (s, 2H), 5.12-5.20 (m, 1H), 4.79-4.89 (m, 1H), 4.66-4.74 (m, 1H), 4.46-4.54 (m. 1H), 4.30-4.38 (m, 2H), 3.62-3.99 (m, 4H), 2.80-3.05 (m, 4H), 2.63-2.75 (m, 2H), 2.36-2.51 (m, 1H). ¹⁹F NMR (377 MHz, DMSO-d₆): δ −63.73, −114.75, −171.26.

### Example 317: 4-[(4-chloro-2-fluorophenyl)methoxy]-5-fluoro-2-[(3S)-3-methyl-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazin-1-yl]pyrimidine (Compound 512a)

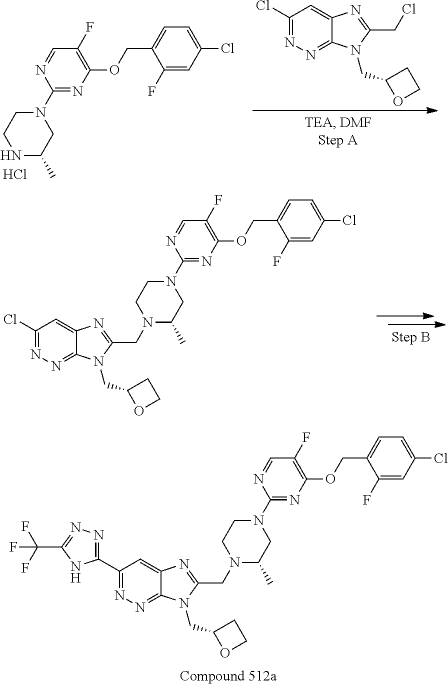 [SMILES: C[C@H]1CN(c2ncc(F)c(OCc3ccc(Cl)cc3F)n2)CCN1 ; Cl ; ClCc1nc2cc(Cl)nnc2n1C[C@@H]1CCO1 ; C[C@H]1CN(c2ncc(F)c(OCc3ccc(Cl)cc3F)n2)CCN1Cc1nc2cc(Cl)nnc2n1C[C@@H]1CCO1 ; C[C@H]1CN(c2ncc(F)c(OCc3ccc(Cl)cc3F)n2)CCN1Cc1nc2cc(-c3nnc(C(F)(F)F)[nH]3)nnc2n1C[C@@H]1CCO1]

### Step A: 3-chloro-6-(((S)-4-(4-((4-chloro-2-fluorobenzyl)oxy)-5-fluoropyrimidin-2-yl)-2-methylpiperazin-1-yl)methyl)-7-(((S)-oxetan-2-yl)methyl)-7H-imidazo[4,5-c]pyridazine

A mixture of (S)-4-((4-chloro-2-fluorobenzyl)oxy)-5-fluoro-2-(3-methylpiperazin-1-yl)pyrimidine HCl salt (430 mg, crude), (S)-3-chloro-6-(chloromethyl)-7-(oxetan-2-ylmethyl)-7H-imidazo[4,5-c]pyridazine (300 mg, 1.1 mmol) and TEA (334 mg, 3.3 mmol) in DMF (3 mL) was stirred at 60° C. for 2 hours. After the reaction was completed, the mixture was diluted with EA (50 mL), washed with H₂O (30 mL*2). The combined organic layer was dried over anhydrous sodium sulfate, filtered and evaporated to dryness. The residue was purified by column chromatography on silica gel (DCM/MeOH=95/5) to give 3-chloro-6-(((S)-4-(4-((4-chloro-2-fluorobenzyl)oxy)-5-fluoropyrimidin-2-yl)-2-methylpiperazin-1-yl)methyl)-7-(((S)-oxetan-2-yl)methyl)-7H-imidazo[4,5-c]pyridazine (370 mg, yield: 57%) as a yellow solid. MS Calcd.: 590.1; MS Found: 591.1 [M+H]⁺.

### Step B: 4-[(4-chloro-2-fluorophenyl)methoxy]-5-fluoro-2-[(3S)-3-methyl-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazin-1-yl]pyrimidine (Compound 512a)

4-[(4-chloro-2-fluorophenyl)methoxy]-5-fluoro-2-[(3S)-3-methyl-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazin-1-yl]pyrimidine (Compound 512a) (12 mg) was obtained as a solid.

MS Calcd.: 691.2; MS Found: 692.1 [M+H]⁺.

¹H NMR (400 MHz, CDCl₃): δ 8.60 (s, 1H), 8.03 (d, J=2.8 Hz, 1H), 7.42 (t, J=8.0 Hz, 1H), 7.09-7.18 (m, 2H), 5.39-5.48 (m, 3H), 4.95-5.06 (m, 3H), 4.76-4.83 (m, 1H), 4.68-4.75 (m. 1H), 4.30-4.50 (m, 3H), 3.90-4.02 (m, 1H), 3.75-3.90 (m, 1H), 3.59-3.70 (m, 1H), 3.45-3.60 (m, 2H), 2.80-2.92 (m, 1H), 2.49-2.60 (m, 1H), 1.49 (d, J=6.4 Hz, 3H). ¹⁹F NMR (377 MHz, CDCl₃): δ −65.26, −115.45, −168.99.

### Example 318: 1-{6-[(4-chloro-2-fluorophenyl)methoxy]-3-fluoropyridin-2-yl}-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazine (Compound 513a)

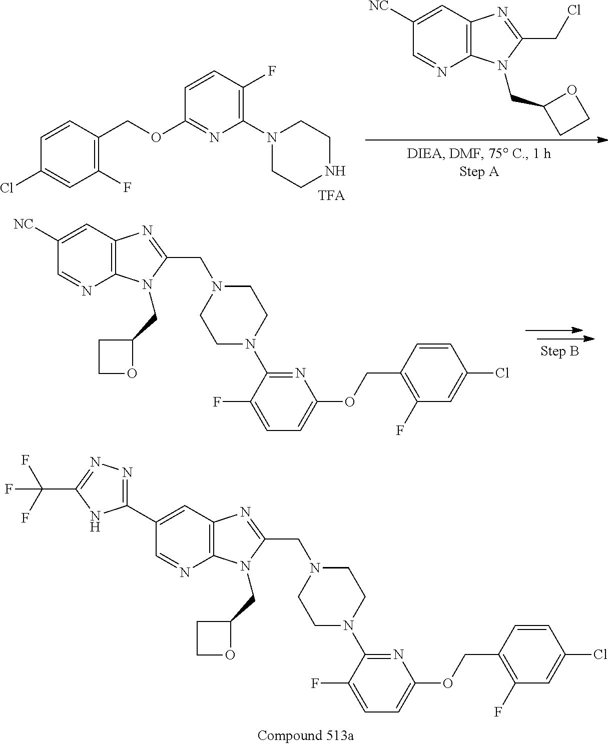

### Step A: (S)-2-((4-(6-((4-chloro-2-fluorobenzyl)oxy)-3-fluoropyridin-2-yl)piperazin-1-yl)methyl)-3-(oxetan-2-ylmethyl)-3H-imidazo[4,5-b]pyridine-6-carbonitrile

To a solution of 1-(6-((4-chloro-2-fluorobenzyl)oxy)-3-fluoropyridin-2-yl)piperazine (104 mg, 0.231 mmol, TFA salt) in DMF (1.5 mL) was added DIEA (81 mg, 0.630 mmol). After 2 minutes, (S)-2-(chloromethyl)-3-(oxetan-2-ylmethyl)-3H-imidazo[4,5-b]pyridine-6-carbonitrile (55 mg, 0.21 mmol) was added. The resulting mixture was heated to 75° C. for an hour. The reaction was quenched with water (10 mL), extracted with ethyl acetate (20 mL×2). The organic layers were combined, dried over Na₂SO₄, filtered and concentrated under vacuum. The residue was purified by column chromatography on silica gel (DCM:MeOH=30:1) to give (S)-2-((4-(6-((4-chloro-2-fluorobenzyl)oxy)-3-fluoropyridin-2-yl)piperazin-1-yl)methyl)-3-(oxetan-2-ylmethyl)-3H-imidazo[4,5-b]pyridine-6-carbonitrile (114 mg, 96% yield) as a brown solid. MS Calcd.: 565.2; MS Found: 566.2 [M+H]⁺.

### Step B: 1-{6-[(4-chloro-2-fluorophenyl)methoxy]-3-fluoropyridin-2-yl}-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazine (Compound 513a)

1-{6-[(4-chloro-2-fluorophenyl)methoxy]-3-fluoropyridin-2-yl}-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazine (Compound 513a) (20 mg) was obtained as a white solid.

MS Calcd.: 675.2; MS Found: 676.0 [M+H]⁺.

¹H NMR (400 MHz, DMSO-d₆): δ 9.01 (d, J=1.6 Hz, 1H), 8.61 (d, J=1.6 Hz, 1H), 7.42-7.55 (m, 3H), 7.30 (dd, J=8.4 Hz, 2.0 Hz, 1H), 6.25 (d, J=8.8 Hz, 1H), 5.29 (s, 2H), 5.15-5.22 (m, 1H), 4.82-4.90 (m, 1H), 4.70-4.77 (m, 1H), 4.47-4.52 (m, 1H), 4.33-4.41 (m, 1H), 3.88-4.09 (m, 2H), 3.37-3.52 (m, 4H), 2.51-2.75 (m, 5H), 2.42-2.51 (m, 1H). ¹⁹F NMR (377 MHz, DMSO-d₆): δ −63.74, −115.35, −140.53.

### Example 319: (2S)-4-{6-[(4-chloro-2-fluorophenyl)methoxy]-3-fluoropyridin-2-yl}-2-methyl-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 514a)

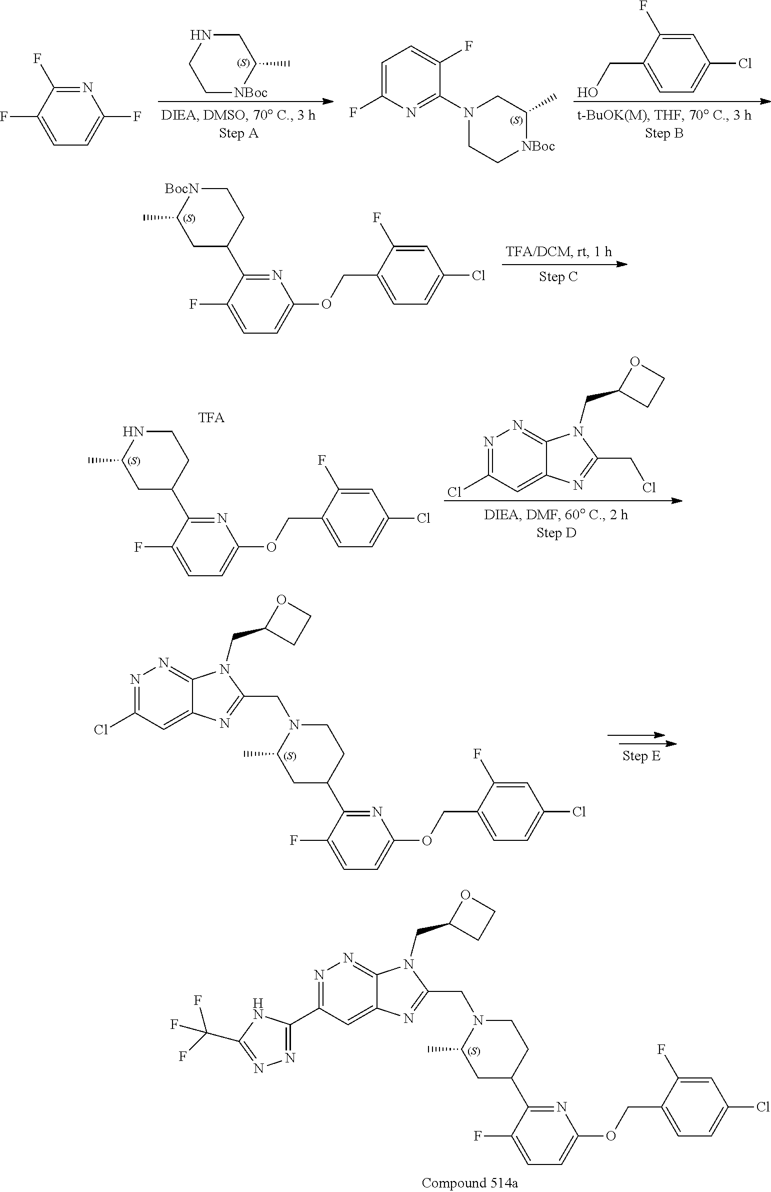

### Step A: tert-butyl (S)-4-(3,6-difluoropyridin-2-yl)-2-methylpiperazine-1-carboxylate

To a solution of 2,3,6-trifluoropyridine (600 mg, 4.5 mmol) in DMSO (8.0 mL) was added tert-butyl (S)-2-methylpiperazine-1-carboxylate (992 mg, 4.96 mmol) and DIEA (873 mg, 6.8 mmol). The mixture was stirred at 70° C. for 3 hours, cooled to room temperature, diluted with ethyl acetate (40 mL), washed with brine (30 mL*2). The organic layer was dried over anhydrous sodium sulfate, filtered and evaporated to dryness. The residue was purified by column chromatography on silica gel (PE/EA=15/1) to give to give tert-butyl (S)-4-(3,6-difluoropyridin-2-yl)-2-methylpiperazine-1-carboxylate (1.2 g, yield: 87%) as colorless oil. MS Calcd.: 313.2; MS Found: 236.1 [M+23]⁺.

### Step B: tert-butyl (S)-4-(6-((4-chloro-2-fluorobenzyl)oxy)-3-fluoropyridin-2-yl)-2-methylpiperazine-1-carboxylate

To a solution of tert-butyl (S)-4-(3,6-difluoropyridin-2-yl)-2-methylpiperazine-1-carboxylate (1.0 g, 3.2 mmol) in THE (20.0 mL) was added (4-chloro-2-fluorophenyl)methanol (767 mg, 4.8 mmol) and t-BuOK (4.8 mL, 4.78 mmol, 1M solution in THF) at rt. The mixture was stirred at 70° C. for 3 hours. After cooled to room temperature, the mixture was diluted with ethyl acetate (60 mL), extracted with H₂O (30 mL*2). The organic layer was dried over anhydrous sodium sulfate, filtered and evaporated to dryness. The residue was purified by column chromatography on silica gel (PE/EA=5/1) to give to give tert-butyl (S)-4-(6-((4-chloro-2-fluorobenzyl)oxy)-3-fluoropyridin-2-yl)-2-methylpiperazine-1-carboxylate (1.1 g, yield: 76%) as colorless oil. MS Calcd.: 453.2; MS Found: 476.1 [M+23]⁺.

### Step C: (S)-1-(6-((4-chloro-2-fluorobenzyl)oxy)-3-fluoropyridin-2-yl)-3-methylpiperazine TFA salt

To a solution of tert-butyl (S)-4-(6-((4-chloro-2-fluorobenzyl)oxy)-3-fluoropyridin-2-yl)-2-methylpiperazine-1-carboxylate (840 mg, 1.8 mmol) in DCM (12 mL) was added TFA (3.0 mL). The solution was stirred at room temperature for an hour. The solvent was removed in vacuo to give (S)-1-(6-((4-chloro-2-fluorobenzyl)oxy)-3-fluoropyridin-2-yl)-3-methylpiperazine TFA salt (1.1 g, crude) as yellow oil. MS Calcd.: 353.1; MS Found: 354.0 [M+H]⁺.

### Step D: 3-chloro-6-(((S)-4-(6-((4-chloro-2-fluorobenzyl)oxy)-3-fluoropyridin-2-yl)-2-methylpiperazin-1-yl)methyl)-7-(((S)-oxetan-2-yl)methyl)-7H-imidazo[4,5-c]pyridazine

To a solution of (S)-1-(6-((4-chloro-2-fluorobenzyl)oxy)-3-fluoropyridin-2-yl)-3-methylpiperazine TFA salt (1.1 g, crude) in DMF (10 mL) was added DIEA (1.4 g, 11.0 mmol). After 2 minutes, (S)-3-chloro-6-(chloromethyl)-7-(oxetan-2-ylmethyl)-7H-imidazo[4,5-c]pyridazine (300 mg, 1.1 mmol) was added. The resulting mixture was stirred at 60° C. for 2 hrs. After the reaction was completed, the mixture was evaporated and the residue purified by column chromatography on silica gel (DCM/MeOH=30/1) to give 3-chloro-6-(((S)-4-(6-((4-chloro-2-fluorobenzyl)oxy)-3-fluoropyridin-2-yl)-2-methylpiperazin-1-yl)methyl)-7-(((S)-oxetan-2-yl)methyl)-7H-imidazo[4,5-c]pyridazine (580 mg, yield: 89%) as yellow oil. MS Calcd.: 589.2; MS Found: 590.1 [M+H]⁺.

### Step E: (2S)-4-{6-[(4-chloro-2-fluorophenyl)methoxy]-3-fluoropyridin-2-yl}-2-methyl-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 514a)

(2S)-4-{6-[(4-chloro-2-fluorophenyl)methoxy]-3-fluoropyridin-2-yl}-2-methyl-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 514a) (20 mg) was obtained as a white solid.

MS Calcd.: 690.2; MS Found: 691.1 [M+H]⁺.

¹H NMR (400 MHz, DMSO-d₆): δ 8.52 (s, 1H), 7.42-7.52 (m, 3H), 7.29 (dd, J=8.4 Hz, 2.0 Hz, 1H), 6.24 (dd, J=8.4 Hz, 2.0 Hz, 1H), 5.25-5.35 (m, 3H), 4.95-5.00 (m, 2H), 4.56 (d, J=14.4 Hz, 1H), 4.47-4.54 (m, 1H), 4.25-4.32 (m, 1H), 3.82 (d, J=14.4 Hz, 1H), 3.62-3.77 (m, 2H), 3.07-3.18 (m, 1H), 2.86-2.95 (m, 1H), 2.67-2.79 (m, 3H), 2.40-2.52 (m, 2H), 1.14 (d, J=6.0 Hz, 3H). ¹⁹F NMR (377 MHz, DMSO-d₆): δ −63.57, −115.40, −140.73.

### Example 320: 2-[(4-chloro-2-fluorophenyl)methoxy]-3-fluoro-6-[(3,4-trans)-3-fluoro-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl]pyridine (Compound 515a)

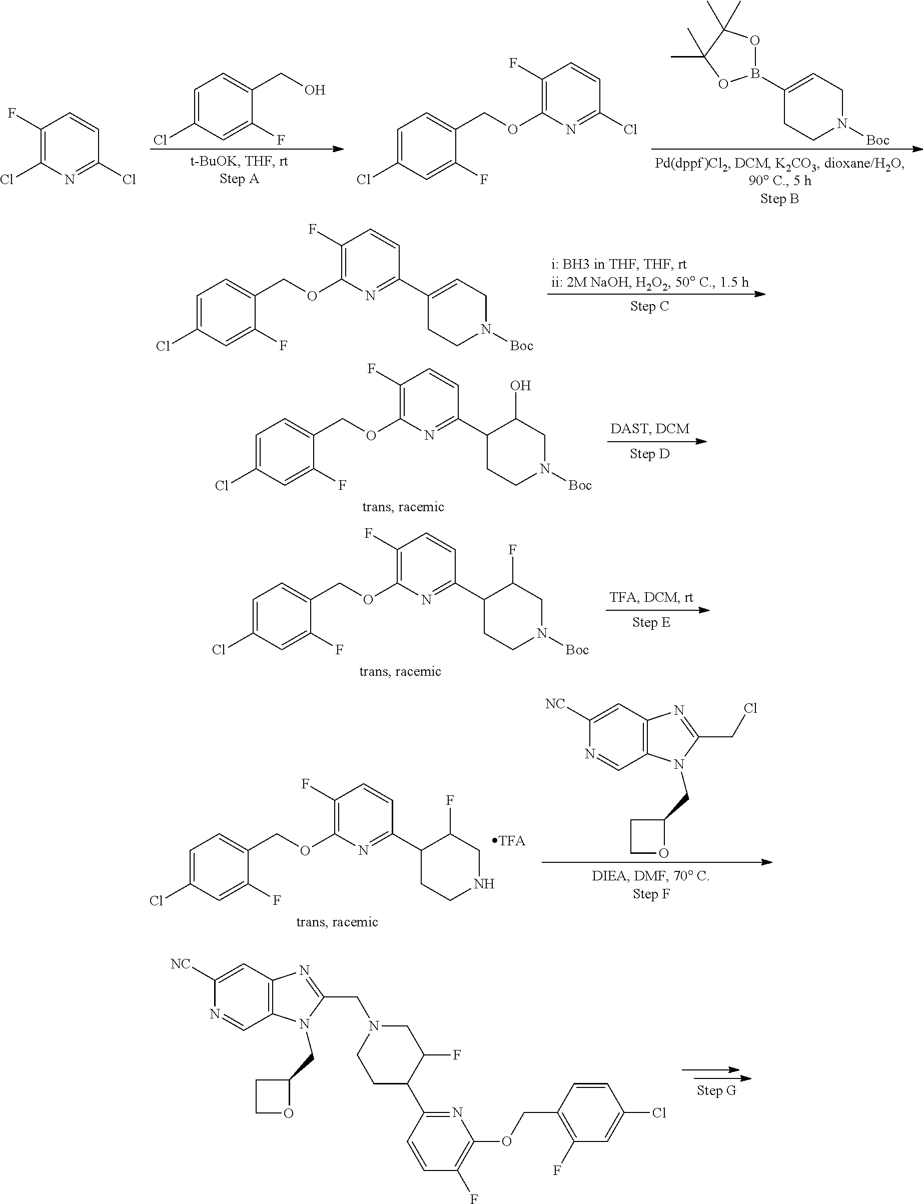

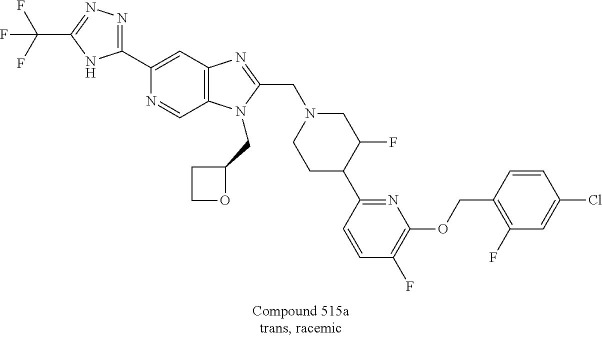 [SMILES: Fc1cc(Cl)ccc1COc1nc(C2CCN(Cc3nc4cc(-c5nnc(C(F)(F)F)[nH]5)ncc4n3C[C@@H]3CCO3)CC2F)ccc1F]

### Step A: 6-chloro-2-((4-chloro-2-fluorobenzyl)oxy)-3-fluoropyridine

To a solution of 2,6-dichloro-3-fluoropyridine (5.0 g, 30.1 mmol), (4-chloro-2-fluorophenyl)methanol (5.8 g, 36.1 mmol) in THE (200 mL) was added t-BuOK (54.2 mL, 54.2 mmol, 1M in THF) at 0° C. and stirred at room temperature for 2 h. The resulting mixture diluted with EA (300 mL) and washed with aq. NH₄Cl (200 mL*2). The organic layer was dried over Na₂SO₄, filtered and concentrated. The residue was purified by column chromatography on silica gel (PE) to give 6-chloro-2-((4-chloro-2-fluorobenzyl)oxy)-3-fluoropyridine (4.0 g, 46% yield) as a white solid. MS Calcd.: 289.0; MS Found: 290.0 [M+H]⁺.

### Step B: tert-butyl 6-((4-chloro-2-fluorobenzyl)oxy)-5-fluoro-3′,6′-dihydro-[2,4′-bipyridine]-1′(2′H)-carboxylate

A mixture of 6-chloro-2-((4-chloro-2-fluorobenzyl)oxy)-3-fluoropyridine (1.0 g, 3.4 mmol), tert-butyl 4-(4,4,5,5-tetramethyl-1,3,2-dioxaborolan-2-yl)-3,6-dihydropyridine-1(2H)-carboxylate (1.3 g, 4.1 mmol), Pd(dppf)Cl₂·DCM (138 mg, 0.17 mmol), K₂CO₃ (938 mg, 6.8 mmol) in dioxane (20 mL), H₂O (2 mL) was stirred at 90° C. for 5 hrs under N₂. The resulting mixture was concentrated and purified by column chromatography on silica gel (PE/EA=15/1) to give tert-butyl 6-((4-chloro-2-fluorobenzyl)oxy)-5-fluoro-3′,6′-dihydro-[2,4′-bipyridine]-1′(2′H)-carboxylate (1.0 g, 66% yield) as colorless oil. MS Calcd.: 436.1; MS Found: 437.0 [M+H]⁺.

### Step C: tert-butyl (3,4-trans)-4-{6-[(4-chloro-2-fluorophenyl)methoxy]-5-fluoropyridin-2-yl}-3-hydroxypiperidine-1-carboxylate

To a solution of tert-butyl 6-((4-chloro-2-fluorobenzyl)oxy)-5-fluoro-3′,6′-dihydro-[2,4′-bipyridine]-1′(2′H)-carboxylate (1.0 g, 2.3 mmol) in THE (10 mL) was added BH₃ (4.6 mL, 4.6 mmol, 1 M in THF) at room temperature, and the mixture was stirred for 3 h. Then 2 M NaOH aqueous solution (3.5 mL, 6.9 mmol) was added, the mixture was stirred at room temperature for 10 mins, Hydrogen peroxide (3.5 mL, 30% aqueous solution) was added, the mixture was heated to 50° C. and stirred for 1.5 h. The resulting mixture diluted with EA (100 mL) and washed with water (50 mL*2). The organic layer was dried over Na₂SO₄, filtered and concentrated. The residue was purified by column chromatography on silica gel (PE/EA=4/1) to give tert-butyl (3,4-trans)-4-{6-[(4-chloro-2-fluorophenyl)methoxy]-5-fluoropyridin-2-yl}-3-hydroxypiperidine-1-carboxylate (560 mg, 55% yield) as colorless oil. MS Calcd.: 454.2; MS Found: 455.4 [M+H]⁺.

¹H NMR (400 MHz, DMSO-d₆): δ 7.45 (t, J=8.4 Hz, 1H), 7.33 (dd, J=10.0 Hz, 8.0 Hz, 1H), 7.11-7.19 (m, 2H), 6.79 (dd, J=8.0 Hz, 2.8 Hz, 1H), 5.40-5.50 (m, 2H), 4.32-4.42 (m, 1H), 4.13-4.26 (m, 1H), 3.84 (dt, J=10.0 Hz, 4.8 Hz, 1H), 2.60-2.84 (m, 3H), 1.64-1.89 (m, 2H), 1.49 (s, 9H).

### Step D: tert-butyl (3,4-trans)-4-{6-[(4-chloro-2-fluorophenyl)methoxy]-5-fluoropyridin-2-yl}-3-fluoropiperidine-1-carboxylate

To a solution of tert-butyl (3,4-trans)-4-{6-[(4-chloro-2-fluorophenyl)methoxy]-5-fluoropyridin-2-yl}-3-hydroxypiperidine-1-carboxylate (560 mg, 1.2 mmol) in DCM (33 mL) was added DAST (244 mg, 1.8 mmol) at 0° C. and stirred for 1 h. The mixture was diluted with EA (100 mL) and wished with water (50 mL×2). The organic layer was dried over Na₂SO₄, filtered and evaporated to dryness. The residue was purified by column chromatography on silica gel (PE:EA=10:1) to give tert-butyl (3,4-trans)-4-{6-[(4-chloro-2-fluorophenyl)methoxy]-5-fluoropyridin-2-yl}-3-fluoropiperidine-1-carboxylate (430 mg, 77% yield) as colorless oil. MS Calcd.: 456.2; MS Found: 401.3 [M-tBu+H]⁺.

¹H NMR (400 MHz, DMSO-d₆): δ 7.45 (t, J=8.4 Hz, 1H), 7.28 (dd, J=9.6 Hz, 7.6 Hz, 1H), 7.11-7.17 (m, 2H), 6.77 (dd, J=8.0 Hz, 2.8 Hz, 1H), 5.42-5.52 (m, 2H), 4.79 (dt, J=10.0 Hz, 5.2 Hz, 0.5H), 4.67 (dt, J=10.0 Hz, 5.6 Hz, 0.5H), 4.40-4.55 (m, 1H), 4.05-4.20 (m, 1H), 2.75-2.90 (m, 3H), 1.78-1.85 (m, 2H), 1.50 (s, 9H).

### Step E: 2-[(4-chloro-2-fluorophenyl)methoxy]-3-fluoro-6-[(3,4-trans)-3-fluoropiperidin-4-yl]pyridine

A mixture of tert-butyl (3,4-trans)-4-{6-[(4-chloro-2-fluorophenyl)methoxy]-5-fluoropyridin-2-yl}-3-fluoropiperidine-1-carboxylate (430 mg, 0.94 mmol), TFA (01 mL) in DCM (4 mL) was stirred at room temperature for 2 h. After the reaction was completed, the mixture was diluted with EA (40 mL) and wished sodium bicarbonate aqueous solution (20 mL*2). The organic layer was dried over Na₂SO₄, filtered and concentrated in vacuum to give 2-[(4-chloro-2-fluorophenyl)methoxy]-3-fluoro-6-[(3,4-trans)-3-fluoropiperidin-4-yl]pyridine (310 mg) as yellow oil. MS Calcd.: 356.1; MS Found: 357.3 [M+H]⁺.

### Step F: 2-[(4-chloro-2-fluorophenyl)methoxy]-3-fluoro-6-[(3,4-trans)-3-fluoro-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl]pyridine (Compound 515a)

2-[(4-chloro-2-fluorophenyl)methoxy]-3-fluoro-6-[(3,4-trans)-3-fluoro-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl]pyridine (Compound 515a) (54 mg) was obtained as a white solid.

MS Calcd.: 692.2; MS Found: 693.1 [M+H]⁺.

¹H NMR (400 MHz, DMSO-d₆): δ 9.25 (d, J=0.8 Hz, 1H), 8.37 (s, 1H), 7.59-7.70 (m, 2H), 7.49 (dd, J=10.0 Hz, 2.0 Hz, 1H), 7.34 (dd, J=8.4 Hz, 2.0 Hz, 1H), 7.04 (dd, J=8.0 Hz, 2.8 Hz, 1H), 5.51 (s, 2H), 4.98-5.21 (m, 2H), 4.83-4.98 (m, 1H), 4.72-4.81 (m, 1H), 4.38-4.61 (m, 4H), 3.58-3.78 (m, 1H), 3.19-3.40 (m, 1H), 2.68-3.15 (m, 4H), 2.30-2.51 (m, 1H), 1.89-2.05 (m, 2H). ¹⁹F NMR (377 MHz, DMSO-d₆): δ −63.73, −115.00, −115.01, −142.71.

### Example 321: 1-{6-[(4-chloro-2-fluorophenyl)methoxy]-5-fluoropyridin-2-yl}-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazine (Compound 516a)

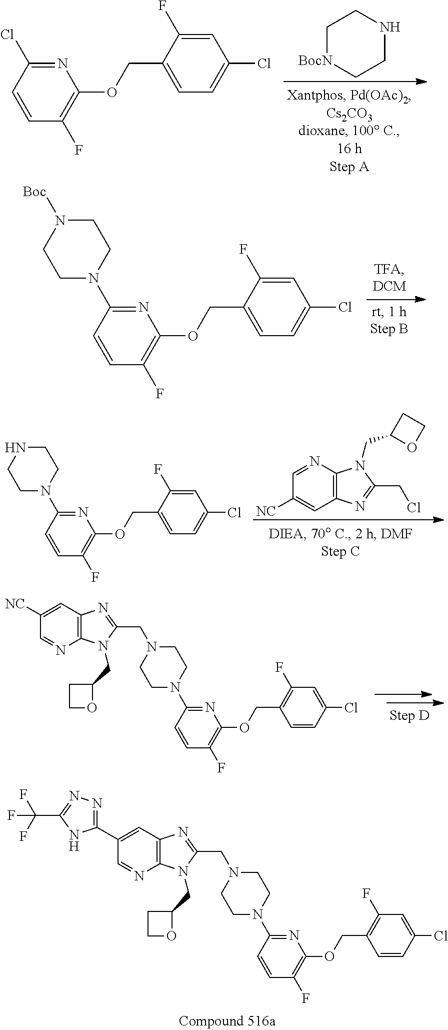

### Step A: tert-butyl 4-(6-((4-chloro-2-fluorobenzyl)oxy)-5-fluoropyridin-2-yl)piperazine-1-carboxylate

To a solution of 6-chloro-2-((4-chloro-2-fluorobenzyl)oxy)-3-fluoropyridine (2.0 g, 6.9 mmol) in dioxane (50 mL) was added Xantphos (1.2 g, 2.1 mmol), Pd(OAc)₂ (156 mg, 0.69 mmol), Cs₂CO₃ (4.5 g, 13.8 mmol) and tert-butyl piperazine-1-carboxylate (2.57 g, 13.817 mmol). The mixture was stirred at 100° C. for 16 hours under N₂. The reaction was quenched with H₂O (50 mL), extracted with ethyl acetate (70 mL×2). The organic layer was dried over anhydrous sodium sulfate, filtered and evaporated to dryness. The residue was purified by column chromatography on silica gel (PE/EA=10/1) to give tert-butyl 4-(6-((4-chloro-2-fluorobenzyl)oxy)-5-fluoropyridin-2-yl)piperazine-1-carboxylate (1.3 g, 43% yield) as a white solid. MS Calcd.: 439.2; MS Found: 440.1 [M+H]⁺.

### Step B: 1-(6-((4-chloro-2-fluorobenzyl)oxy)-5-fluoropyridin-2-yl)piperazine

To a solution of tert-butyl 4-(6-((4-chloro-2-fluorobenzyl)oxy)-5-fluoropyridin-2-yl)piperazine-1-carboxylate (201 mg, 0.46 mmol) in DCM (2 mL) was added TFA (0.5 mL). The solution was stirred at rt for 1 hour. The solvent was removed in vacuo to give 1-(6-((4-chloro-2-fluorobenzyl)oxy)-5-fluoropyridin-2-yl)piperazine TFA salt (255 mg, crude) as a yellow oil. MS Calcd.: 339.1; MS Found: 340.1 [M+H]⁺.

### Step C: (S)-2-((4-(6-((4-chloro-2-fluorobenzyl)oxy)-5-fluoropyridin-2-yl)piperazin-1-yl)methyl)-3-(oxetan-2-ylmethyl)-3H-imidazo[4,5-b]pyridine-6-carbonitrile

A mixture of 1-(6-((4-chloro-2-fluorobenzyl)oxy)-5-fluoropyridin-2-yl)piperazine TFA salt (155 mg, crude), (S)-2-(chloromethyl)-3-(oxetan-2-ylmethyl)-3H-imidazo[4,5-b]pyridine-6-carbonitrile (100 mg, 0.38 mmol), DIEA (492 mg, 3.814 mmol) in DMF (3 mL) was stirred at 70° C. for 2 hours. After the reaction was completed, the mixture was diluted with EA (60 mL), washed with H₂O (30 mL*2). The combined organic layer was dried over anhydrous sodium sulfate, filtered, and evaporated to dryness. The residue was purified by column chromatography on silica gel (PE/EA=1/1) to give (S)-2-((4-(6-((4-chloro-2-fluorobenzyl)oxy)-5-fluoropyridin-2-yl)piperazin-1-yl)methyl)-3-(oxetan-2-ylmethyl)-3H-imidazo[4,5-b]pyridine-6-carbonitrile (151 mg, yield: 70%) as a yellow oil. MS Calcd.: 565.2; MS Found: 566.1 [M+H]⁺.

### Step D: 1-{6-[(4-chloro-2-fluorophenyl)methoxy]-5-fluoropyridin-2-yl}-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazine (Compound 516a)

A mixture of (S)-3-(2-((4-(6-((4-chloro-2-fluorobenzyl)oxy)-5-fluoropyridin-2-yl)piperazin-1-yl)methyl)-3-(oxetan-2-ylmethyl)-3H-imidazo[4,5-b]pyridin-6-yl)-5,5,5-trifluoro-5l7-1,2,4-oxadiazole (138 mg, 0.20 mmol) and N₂H₄·H₂O (41 mg, 0.82 mmol) in DMF (1 mL) was stirred at rt for 1 hour. After the reaction was completed, the mixture was purified directly by Prep-HPLC (0.1% FA/H₂O/CH₃CN) to give 1-{6-[(4-chloro-2-fluorophenyl)methoxy]-5-fluoropyridin-2-yl}-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazine (Compound 516a) (26 mg, yield: 19%) as a white solid.

MS Calcd.: 675.2; MS Found: 676.0 [M+H]⁺.

¹H NMR (400 MHz, DMSO-d₆): δ 9.00 (d, J=1.6 Hz, 1H), 8.58 (d, J=1.6 Hz, 1H), 7.50-7.58 (m, 1H), 7.42-7.50 (m, 2H), 7.31 (dd, J=8, 1.6 Hz, 1H), 6.26-6.32 (m, 1H), 5.39 (s, 2H), 5.14-5.24 (m, 1H), 4.80-4.89 (m, 1H), 4.68-4.76 (m, 1H), 4.46-4.53 (m, 1H), 4.34-4.42 (m, 1H), 3.92-4.04 (m, 2H), 3.38-3.50 (m, 5H), 2.65-2.75 (m, 1H), 2.55-2.63 (m, 4H). ¹⁹F NMR (377 MHz, DMSO-d₆): δ −63.15, −115.20, −156.73.

### Example 322: 3-fluoro-4-{[(3-fluoro-6-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyridin-2-yl)oxy]methyl}benzonitrile (Compound 517a)

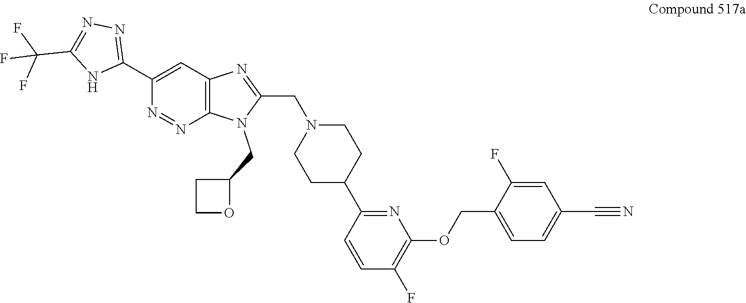 [SMILES: N#Cc1ccc(COc2nc(C3CCN(Cc4nc5cc(-c6nnc(C(F)(F)F)[nH]6)nnc5n4C[C@@H]4CCO4)CC3)ccc2F)c(F)c1]

3-fluoro-4-{[(3-fluoro-6-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}pyridin-2-yl)oxy]methyl}benzonitrile (Compound 517a) (13 mg) was obtained as a white solid.

MS Calcd.: 666.2; MS Found: 667.2 [M+H]⁺.

¹H NMR (400 MHz, DMSO-d₆): δ 8.52 (s, 1H), 7.90 (d, J=10.4 Hz, 1H), 7.69-7.76 (m, 2H), 7.58-7.65 (m, 1H), 6.92 (dd, J=9.4 Hz, 2.8 Hz, 1H), 5.56 (s, 2H), 5.25-5.34 (m, 1H), 5.01-5.09 (m, 1H), 4.87-4.96 (m, 1H), 4.48-4.56 (m, 1H), 4.39-4.46 (m, 1H), 4.00-4.11 (m, 2H), 2.92-3.03 (m, 2H), 2.70-2.81 (m, 1H), 2.52-2.70 (m, 2H), 2.23-2.35 (m, 2H), 1.62-1.83 (m, 4H). ¹⁹F NMR (377 MHz, DMSO-d₆): δ −63.62, −115.29, −143.98.

### Example 323: (2S)-4-{3-[(4-chloro-2-fluorophenyl)methoxy]-4-fluorophenyl}-2-methyl-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 518a)

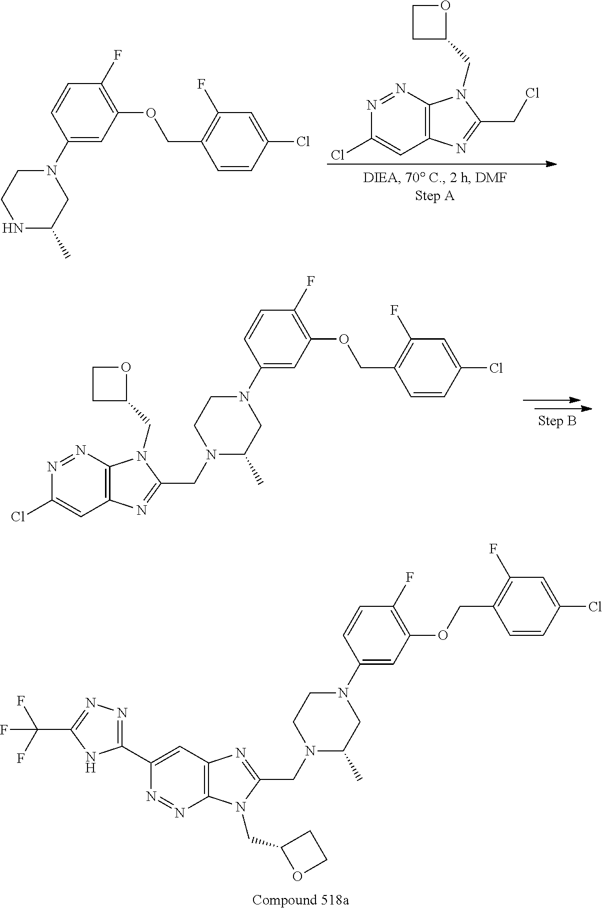

### Step A: 3-chloro-6-(((S)-4-(3-((4-chloro-2-fluorobenzyl)oxy)-4-fluorophenyl)-2-methylpiperazin-1-yl)methyl)-7-(((S)-oxetan-2-yl)methyl)-7H-imidazo[4,5-c]pyridazine

To a solution of (S)-1-(3-((4-chloro-2-fluorobenzyl)oxy)-4-fluorophenyl)-3-methylpiperazine TFA Salt (466 mg, crude) in DMF (10 mL) was added DIEA (1.4 g, 11.0 mmol) and (S)-3-chloro-6-(chloromethyl)-7-(oxetan-2-ylmethyl)-7H-imidazo[4,5-c]pyridazine (300 mg, 1.103 mmol). The solution was stirred at 70° C. for 2 hours. The reaction was quenched with H₂O (50 mL), extracted with ethyl acetate (70 mL×2). The combined organic layer was dried over anhydrous sodium sulfate, filtered and evaporated to dryness. The residue was purified by column chromatography on silica gel (PE/EA=1/2) to give 3-chloro-6-(((S)-4-(3-((4-chloro-2-fluorobenzyl)oxy)-4-fluorophenyl)-2-methylpiperazin-1-yl)methyl)-7-(((S)-oxetan-2-yl)methyl)-7H-imidazo[4,5-c]pyridazine (329 mg, 51% yield) as a red solid. MS Calcd.: 588.2; MS Found: 589.2 [M+H]⁺.

### Step B: (2S)-4-{3-[(4-chloro-2-fluorophenyl)methoxy]-4-fluorophenyl}-2-methyl-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 518a)

(2S)-4-{3-[(4-chloro-2-fluorophenyl)methoxy]-4-fluorophenyl}-2-methyl-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 518a) (7.8 mg) was obtained as a white solid.

MS Calcd.: 689.2; MS Found: 690.1 [M+H]⁺.

¹H NMR (400 MHz, DMSO-d₆): δ 8.51 (s, 1H), 7.59 (t, J=8.0 Hz, 1H), 7.50 (dd, J=10.0, 2.0 Hz, 1H), 7.35 (dd, J=8.4 Hz, 2.0 Hz, 1H), 7.00-7.07 (m, 1H), 6.81-6.87 (m, 1H), 6.44-6.50 (m, 1H), 5.27-5.36 (m, 1H), 5.18 (s, 2H), 4.95-5.00 (m, 2H), 4.61 (d, J=14.4 Hz, 1H), 4.45-4.53 (m, 1H), 4.23-4.31 (m, 1H), 3.81 (d, J=14.4 Hz, 1H), 3.48-3.52 (m, 1H), 3.36-3.39 (m, 1H), 3.26-3.32 (m, 1H), 2.68-2.82 (m, 4H), 2.48-2.60 (m, 2H), 1.19 (d, J=6.0 Hz, 3H). ¹⁹F NMR (277 MHz, DMSO-d₆): δ −63.45, −63.47, −63.48, −63.51, −115.00, −146.13.

### Example 324: (2S)-4-{6-[(4-chloro-2-fluorophenyl)methoxy]pyridin-2-yl}-2-methyl-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 519a)

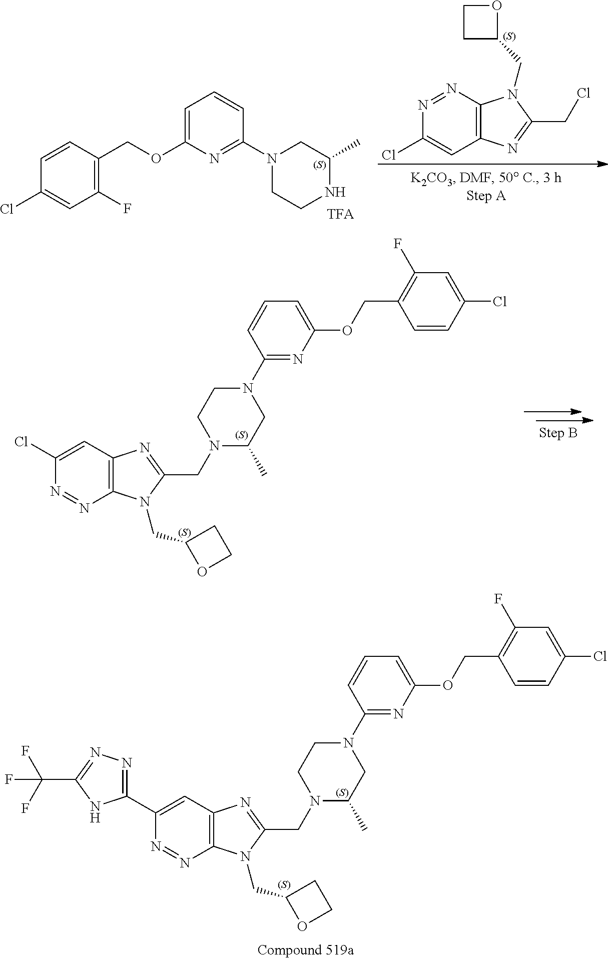

### Step A: 3-chloro-6-(((S)-4-(6-((4-chloro-2-fluorobenzyl)oxy)pyridin-2-yl)-2-methylpiperazin-1-yl)methyl)-7-(((S)-oxetan-2-yl)methyl)-7H-imidazo[4,5-c]pyridazine

To a mixture of (S)-1-(6-((4-chloro-2-fluorobenzyl)oxy)pyridin-2-yl)-3-methylpiperazine TFA salt (1.0 g, crude) in DMF (10 mL) was added K₂CO₃ (2.8 g, 20.0 mmol). After 2 minutes, (S)-3-chloro-6-(chloromethyl)-7-(oxetan-2-ylmethyl)-7H-imidazo[4,5-c]pyridazine (310 mg, 1.14 mmol) was added. The resulting mixture was heated to 50° C. for 3 hours. The reaction was quenched with water (90 mL), extracted with ethyl acetate (80 mL×2). The organic layers were combined, dried over Na₂SO₄, filtered and concentrated under vacuum. The residue was purified by flash chromatography (PE:EA=2:1) to give 3-chloro-6-(((S)-4-(6-((4-chloro-2-fluorobenzyl)oxy)pyridin-2-yl)-2-methylpiperazin-1-yl)methyl)-7-(((S)-oxetan-2-yl)methyl)-7H-imidazo[4,5-c]pyridazine (535 mg, 82% yield) as a yellow solid. MS Calcd.: 571.2; MS Found: 572.2 [M+H]⁺.

### Step B: (2S)-4-{6-[(4-chloro-2-fluorophenyl)methoxy]pyridin-2-yl}-2-methyl-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 519a)

(2S)-4-{6-[(4-chloro-2-fluorophenyl)methoxy]pyridin-2-yl}-2-methyl-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazine (Compound 519a) (3.7 mg) was obtained as a white solid.

MS Calcd.: 672.2; MS Found: 673.1 [M+H]⁺.

¹H NMR (400 MHz, DMSO-d₆): δ 8.55 (s, 1H), 7.40-7.60 (m, 3H), 7.29 (dd, J=8.0 Hz, 1.6 Hz, 1H), 6.38 (d, J=8.4 Hz, 1H), 6.11 (d, J=8.4 Hz, 1H), 5.23-5.38 (m, 3H), 4.92-5.02 (m, 2H), 4.47-4.70 (m, 2H), 4.26-4.35 (m, 1H), 4.73-4.10 (m, 3H), 2.67-3.10 (m, 7H), 1.11-1.30 (m, 3H). ¹⁹F NMR (377 MHz, DMSO-d₆): δ −63.67, −115.45.

### Example 325: 4-{3-[(4-chloro-2-fluorophenyl)methoxy]-4-fluorophenyl}-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 520a)

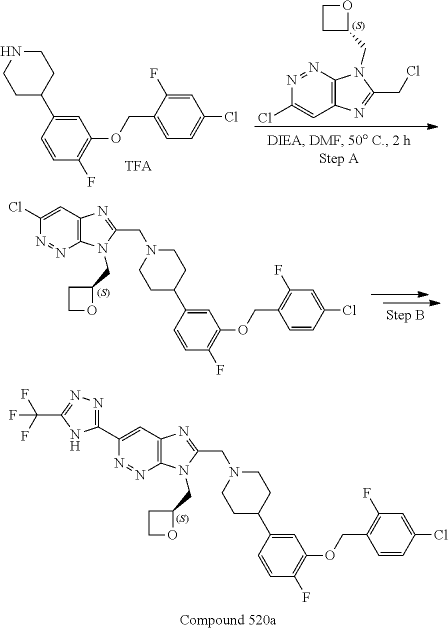

### Step A: (S)-3-chloro-6-((4-(3-((4-chloro-2-fluorobenzyl)oxy)-4-fluorophenyl)piperidin-1-yl)methyl)-7-(oxetan-2-ylmethyl)-7H-imidazo[4,5-c]pyridazine

To a solution of 4-(3-((4-chloro-2-fluorobenzyl)oxy)-4-fluorophenyl)piperidine TFA salt (735 mg, crude) in DMF (10 mL) was added DIEA (2.8 g, 20.0 mmol). After 2 minutes, (S)-3-chloro-6-(chloromethyl)-7-(oxetan-2-ylmethyl)-7H-imidazo[4,5-c]pyridazine (350 mg, 1.3 mmol) was added. The resulting mixture was heated to 50° C. for 2 hours. The reaction was quenched with water (90 mL), extracted with ethyl acetate (80 mL×2). The organic layers were combined, dried over Na₂SO₄, filtered and concentrated under vacuum. The residue was purified by flash chromatography (PE:EA=4:1) to give (S)-3-chloro-6-((4-(3-((4-chloro-2-fluorobenzyl)oxy)-4-fluorophenyl)piperidin-1-yl)methyl)-7-(oxetan-2-ylmethyl)-7H-imidazo[4,5-c]pyridazine (600 mg, 81% yield) as a yellow solid. MS Calcd.: 573.2; MS Found: 574.1 [M+H]⁺.

### Step B: 4-{3-[(4-chloro-2-fluorophenyl)methoxy]-4-fluorophenyl}-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 520a)

4-{3-[(4-chloro-2-fluorophenyl)methoxy]-4-fluorophenyl}-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 520a) (12 mg) was obtained as a white solid.

MS Calcd.: 674.2; MS Found: 675.1 [M+H]⁺.

¹H NMR (400 MHz, DMSO-d₆): δ 8.51 (s, 1H), 7.60 (t, J=8.0 Hz, 1H), 7.50 (d, J=10.0 Hz, 1H), 7.35 (d, J=8.0 Hz, 1H), 7.08-7.20 (m, 2H), 6.82-6.88 (m, 1H), 5.23-5.34 (m, 1H), 5.20 (s, 2H), 5.00-5.08 (m, 1H), 4.87-4.95 (m, 1H), 4.49-4.57 (m, 1H), 4.37-4.46 (m, 1H), 4.02-4.12 (m, 3H), 2.95-3.07 (m, 2H), 2.70-2.85 (m, 2H), 2.25-2.36 (m, 2H), 1.61-1.83 (m, 4H). ¹⁹F NMR (377 MHz, DMSO-d₆): δ −63.49, −115.04, −138.18.

### Example 326: 3-fluoro-4-[(2-fluoro-5-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}phenoxy)methyl]benzonitrile (Compound 521a)

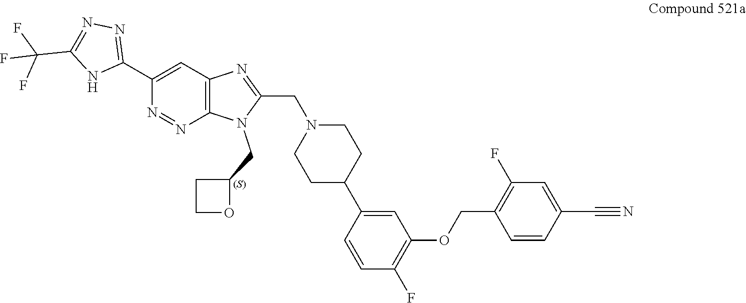

3-fluoro-4-[(2-fluoro-5-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}phenoxy)methyl]benzonitrile (Compound 521a) (11 mg) was obtained as a white solid.

MS Calcd.: 665.2; MS Found: 666.2 [M+H]⁺.

¹H NMR (400 MHz, DMSO-d₆): δ 8.45 (s, 1H), 7.92 (d, J=9.6 Hz, 1H), 7.73-7.81 (m, 2H), 7.10-7.22 (m, 2H), 6.83-6.91 (m, 1H), 5.32 (s, 2H), 5.23-5.29 (m, 1H), 4.97-5.06 (m, 1H), 4.86-4.94 (m, 1H), 4.49-4.58 (m, 1H), 4.36-4.45 (m, 1H), 4.01-4.07 (m, 3H), 2.96-3.06 (m, 2H), 2.70-2.80 (m, 2H), 2.25-2.35 (m, 2H), 1.64-1.83 (m, 4H). ¹⁹F NMR (377 MHz, DMSO-d₆): δ −62.65, −115.16, −138.24.

### Example 327: 3-fluoro-4-({2-fluoro-5-[(3S)-3-methyl-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazin-1-yl]phenoxy}methyl)benzonitrile (Compound 522a)

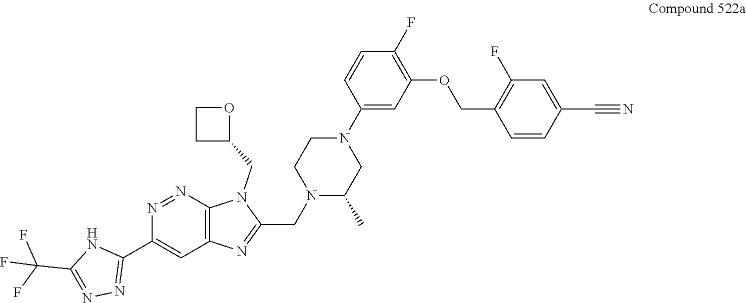 [SMILES: C[C@H]1CN(c2ccc(F)c(OCc3ccc(C#N)cc3F)c2)CCN1Cc1nc2cc(-c3nnc(C(F)(F)F)[nH]3)nnc2n1C[C@@H]1CCO1]

3-fluoro-4-({2-fluoro-5-[(3S)-3-methyl-4-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperazin-1-yl]phenoxy}methyl)benzonitrile (Compound 522a) (9.2 mg) was obtained as a white solid.

MS Calcd.: 680.2; MS Found: 681.0 [M+H]⁺.

¹H NMR (400 MHz, DMSO-d₆): δ 8.52 (s, 1H), 7.92 (d, J=9.2 Hz, 1H), 7.74-7.79 (m, 2H), 7.01-7.10 (m, 1H), 6.82-6.88 (m, 1H), 6.44-6.52 (m, 1H), 5.26-5.35 (m, 3H), 4.95-5.00 (m, 2H), 4.61 (d, J=14.4 Hz, 1H), 4.45-4.53 (m, 1H), 4.24-4.31 (m, 1H), 3.81 (d, J=14.0 Hz, 1H), 3.44-3.54 (m, 2H), 2.63-2.83 (m, 5H), 2.52-2.60 (m, 2H), 1.19 (d, J=6.0 Hz, 3H). ¹⁹F NMR (377 MHz, DMSO-d₆): δ −63.55, −115.13, −146.18.

### Example 328: 4-[(3S)-3-(4-chloro-2-fluorophenyl)-2,3-dihydro-1,4-benzodioxin-5-yl]-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 523a)

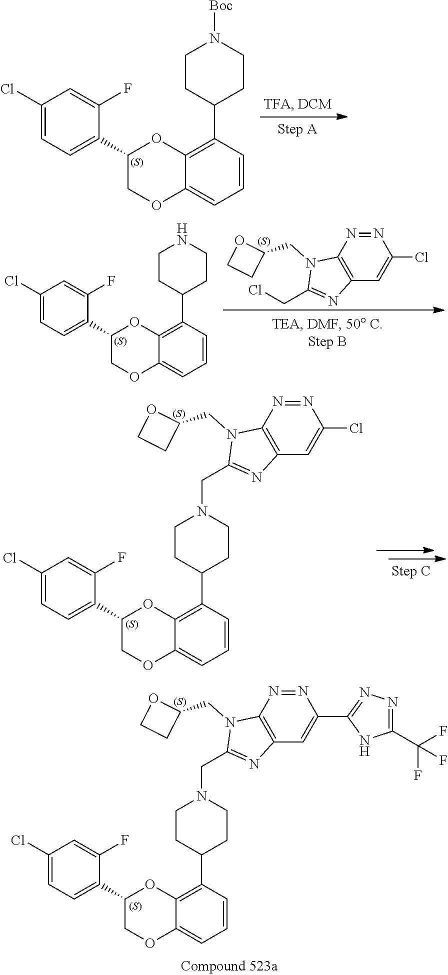

### Step A: (S)-4-(3-(4-chloro-2-fluorophenyl)-2,3-dihydrobenzo[b][1,4]dioxin-5-yl)piperidine TFA salt

To a solution of tert-butyl (S)-4-(3-(4-chloro-2-fluorophenyl)-2,3-dihydrobenzo[b][1,4]dioxin-5-yl)piperidine-1-carboxylate (220 mg, 0.49 mmol) in DCM (3 mL) was added TFA (1 mL). The reaction was stirred at room temperature for 1 hour. After the reaction was completed, the mixture was concentrated in vacuo to give (S)-4-(3-(4-chloro-2-fluorophenyl)-2,3-dihydrobenzo[b][1,4]dioxin-5-yl)piperidine TFA salt (227 mg, crude) as yellow oil. MS Calcd.: 347.1; MS Found: 348.0 [M+H]⁺.

### Step B: 3-chloro-6-((4-((S)-3-(4-chloro-2-fluorophenyl)-2,3-dihydrobenzo[b][1,4]dioxin-5-yl)piperidin-1-yl)methyl)-7-(((S)-oxetan-2-yl)methyl)-7H-imidazo[4,5-c]pyridazine

A mixture of (S)-4-(3-(4-chloro-2-fluorophenyl)-2,3-dihydrobenzo[b][1,4]dioxin-5-yl)piperidine TFA salt (227 mg, crude), (S)-3-chloro-6-(chloromethyl)-7-(oxetan-2-ylmethyl)-7H-imidazo[4,5-c]pyridazine (134 mg, 0.49 mmol) and TEA (150 mg, 1.48 mmol) in DMF (3 mL) was stirred at 50° C. for 2 hour. After the reaction was completed, the mixture was diluted with EA (50 mL), washed with H₂O (30 mL×2). The combined organic layer was dried over anhydrous sodium sulfate, filtered and evaporated to dryness. The residue was purified by column chromatography on silica gel (DCM/MeOH=10/1) to give 3-chloro-6-((4-((S)-3-(4-chloro-2-fluorophenyl)-2,3-dihydrobenzo[b][1,4]dioxin-5-yl)piperidin-1-yl)methyl)-7-(((S)-oxetan-2-yl)methyl)-7H-imidazo[4,5-c]pyridazine (230 mg, yield: 80%) as a yellow solid. MS Calcd.: 583.2; MS Found: 584.2 [M+H]⁺.

### Step C: 4-[(3S)-3-(4-chloro-2-fluorophenyl)-2,3-dihydro-1,4-benzodioxin-5-yl]-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 523a)

4-[(3S)-3-(4-chloro-2-fluorophenyl)-2,3-dihydro-1,4-benzodioxin-5-yl]-1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidine (Compound 523a) (8.2 mg) was obtained as a white solid.

MS Calcd.: 684.2; MS Found: 685.3 [M+H]⁺.

¹H NMR (400 MHz, DMSO-d₆): δ 8.47 (s, 1H), 7.50-7.60 (m, 2H), 7.43 (dd, J=8.4 Hz, J=2.0 Hz, 1H), 6.77-6.88 (m, 3H), 5.44-5.50 (m, 1H), 5.21-5.29 (m, 1H), 4.97-5.05 (m, 1H), 4.83-4.91 (m, 1H), 4.48-4.55 (m, 1H), 4.36-4.48 (m, 2H), 4.05-4.15 (m, 1H), 3.97-4.05 (m, 2H), 2.92-3.03 (m, 2H), 2.80-2.92 (m, 1H), 2.65-2.80 (m, 1H), 2.48-2.58 (m, 1H), 2.21-2.30 (m, 2H), 1.60-1.83 (m, 4H). ¹⁹F NMR (377 MHz, DMSO-d₆): δ −63.13, −115.08.

### Example 329: 5-chloro-2-[4-fluoro-2-methyl-7-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]pyridine (Compound 524a)

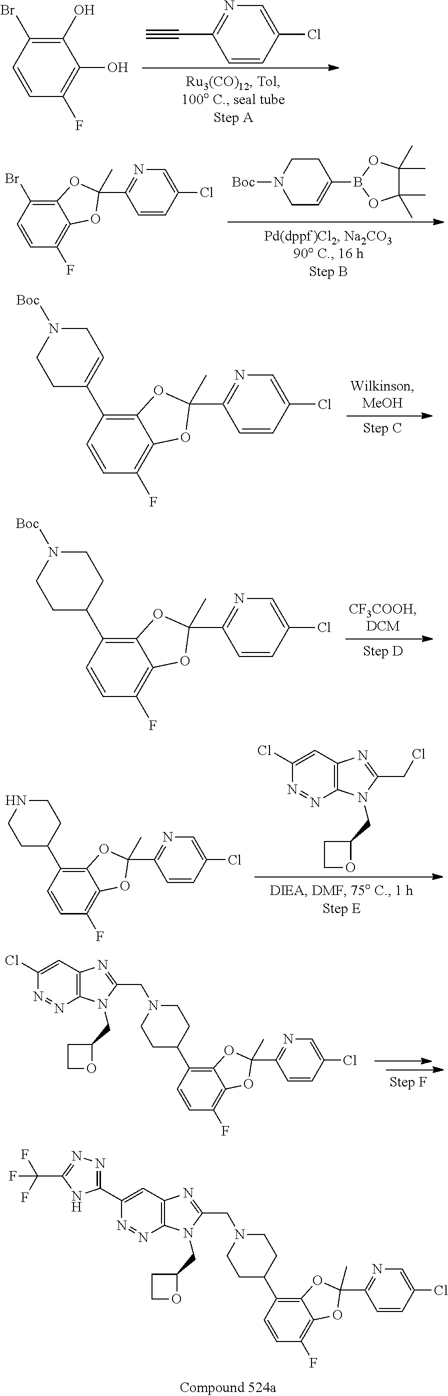

### Step A: 2-(4-bromo-7-fluoro-2-methylbenzo[d][1,3]dioxol-2-yl)-5-chloropyridine

A mixture of 3-bromo-6-fluorobenzene-1,2-diol (2.0 g, 9.7 mmol) and 5-chloro-2-ethynylpyridine (2 g, 14.6 mmol) and Ru₃(CO)₁₋₂ (330 mg, 0.48 mmol) in toluene (5 mL) was degassed and charged with Ar. The reaction was stirred at 100° C. for 16 hours in sealed tube. After the reaction was completed, the reaction was concentrated in vacuum. The residue was purified by column chromatography (PE) to give 2-(4-bromo-7-fluoro-2-methylbenzo[d][1,3]dioxol-2-yl)-5-chloropyridine (1.0 g, 30% yield) as a yellow oil.

¹H NMR (400 MHz, DMSO-d₆): δ 8.77 (d, J=2.0 Hz, 1H), 8.07 (dd, J=2.8 Hz, J=8.4 Hz, 1H), 7.69 (d, J=8.4 Hz, 1H), 7.11 (dd, J=4.4 Hz, J=8.8 Hz, 1H), 6.90 (t, J=9.6 Hz, 1H), 2.14 (s, 3H). ¹⁹F NMR (377 MHz, DMSO-d₆): δ −139.42.

### Step B: tert-butyl 4-(2-(5-chloropyridin-2-yl)-7-fluoro-2-methylbenzo[d][1,3]dioxol-4-yl)-3,6-dihydropyridine-1(2H)-carboxylate

A mixture of 2-(4-bromo-7-fluoro-2-methylbenzo[d][1,3]dioxol-2-yl)-5-chloropyridine (1.0 g, 2.9 mmol) and tert-butyl 4-(4,4,5,5-tetramethyl-1,3,2-dioxaborolan-2-yl)-3,6-dihydropyridine-1(2H)-carboxylate (991 mg, 3.2 mmol), Pd(dppf)Cl₂ (107 mg, 0.15 mmol) and Na₂CO₃ (929 mg, 8.8 mmol) in dioxane/H₂O (30 mL/3 mL) was stirred at 90° C. for 16 hours under N₂. After the reaction was completed, the reaction was quenched with water (50 mL) and extracted with ethyl acetate (100 mL×3). The organic layer was combined and washed with brine (50 mL×2), dried over sodium sulfate, filtered and concentrated in vacuum. The residue was purified by column chromatography on silica gel (PE:EA=10:1) to give tert-butyl 4-(2-(5-chloropyridin-2-yl)-7-fluoro-2-methylbenzo[d][1,3]dioxol-4-yl)-3,6-dihydropyridine-1(2H)-carboxylate (680 mg, 52% yield) as a yellow solid. MS Calcd.: 446.1; MS Found: 391.0 [M-56+H]⁺.

### Step C: tert-butyl 4-(2-(5-chloropyridin-2-yl)-7-fluoro-2-methylbenzo[d][1,3]dioxol-4-yl)piperidine-1-carboxylate

A mixture of tert-butyl 4-(2-(5-chloropyridin-2-yl)-7-fluoro-2-methylbenzo[d][1,3]dioxol-4-yl)-3,6-dihydropyridine-1(2H)-carboxylate (680 mg, 1.5 mmol), Wilkinson's catalyst (70 mg, 0.08 mmol) in MeOH (10 mL) was stirred at 50° C. for 16 hours under H₂ (200 Psi). After the reaction was completed, the reaction was filtered and concentrated in vacuum. The residue was purified by column chromatography on silica gel (PE:EA=10:1) to give tert-butyl 4-(2-(5-chloropyridin-2-yl)-7-fluoro-2-methylbenzo[d][1,3]dioxol-4-yl)piperidine-1-carboxylate (350 mg, 51% yield) as colorless oil. MS Calcd.: 448.2; MS Found: 449.0[M+H]⁺.

### Step D: 5-chloro-2-(4-fluoro-2-methyl-7-(piperidin-4-yl)benzo[d][1,3]dioxol-2-yl)pyridine TFA salt

To a solution of tert-butyl 4-(2-(5-chloropyridin-2-yl)-7-fluoro-2-methylbenzo[d][1,3]dioxol-4-yl)piperidine-1-carboxylate (350 mg, 0.78 mmol) in DCM (2 mL) was added TFA (0.5 mL) at room temperature. The reaction was stirred at room temperature for 1 hour. After the reaction was completed, the reaction was concentrated in vacuum to give 5-chloro-2-(4-fluoro-2-methyl-7-(piperidin-4-yl)benzo[d][1,3]dioxol-2-yl)pyridine TFA salt (420 mg, crude) as yellow oil. MS Calcd.: 348.1; MS Found: 349.1 [M+H]⁺.

### Step E: 3-chloro-6-((4-(2-(5-chloropyridin-2-yl)-7-fluoro-2-methylbenzo[d][1,3]dioxol-4-yl)piperidin-1-yl)methyl)-7-(((S)-oxetan-2-yl)methyl)-7H-imidazo[4,5-c]pyridazine

A solution of 5-chloro-2-(4-fluoro-2-methyl-7-(piperidin-4-yl)benzo[d][1,3]dioxol-2-yl)pyridine TFA salt (420 mg, crude), (S)-3-chloro-6-(chloromethyl)-7-(oxetan-2-ylmethyl)-7H-imidazo[4,5-c]pyridazine (329 mg, 1.2 mmol), DIEA (470 mg, 3.6 mmol) in DMF (5 mL) was stirred at 75° C. for 1 hour. After the reaction was completed, the mixture was diluted with EA (50 mL), washed with H₂O (40 mL×3). The combined organic layer was dried over anhydrous sodium sulfate, filtered and concentrated. The residue was purified by column chromatography on silica gel (DCM/MeOH=20/1) to give 3-chloro-6-((4-(2-(5-chloropyridin-2-yl)-7-fluoro-2-methylbenzo[d][1,3]dioxol-4-yl)piperidin-1-yl)methyl)-7-(((S)-oxetan-2-yl)methyl)-7H-imidazo[4,5-c]pyridazine (367 mg, 80% yield, two steps) as yellow oil. MS Calcd.: 584.2; MS Found: 585.2[M+H]⁺.

### Step F: 5-chloro-2-[4-fluoro-2-methyl-7-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]pyridine (Compound 524a)

5-chloro-2-[4-fluoro-2-methyl-7-{1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}-2H-1,3-benzodioxol-2-yl]pyridine (Compound 524a) (1.1 mg) was obtained as a white solid.

MS Calcd.: 685.2; MS Found: 686.2 [M+H]⁺.

¹H NMR (400 MHz, DMSO-d₆): δ 8.74 (d, J=2.4 Hz, 1H), 8.47 (s, 1H), 8.03 (dd, J=2.0 Hz, J=8.0 Hz, 1H), 7.65 (d, J=8.4 Hz, 1H), 6.75-6.84 (m, 2H), 5.23-5.35 (m, 1H), 4.95-5.05 (m, 1H), 4.83-4.91 (m, 1H), 4.36-4.53 (m, 2H), 3.95-4.10 (m, 2H), 2.93-3.06 (m, 2H), 2.65-2.77 (m, 3H), 2.23-2.36 (m, 2H), 2.07 (s, 3H), 1.67-1.84 (m, 4H). ¹⁹F NMR (377 MHz, DMSO-d₆): δ −63.44, −141.52.

### Example 330: (2S)-4-{6-[(4-chloro-2-fluorophenyl)methoxy]-3-fluoropyridin-2-yl}-2-methyl-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 525a)

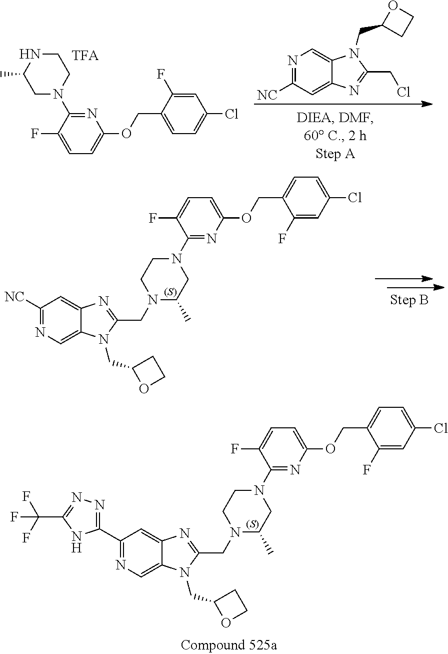

### Step A: 2-(((S)-4-(6-((4-chloro-2-fluorobenzyl)oxy)-3-fluoropyridin-2-yl)-2-methylpiperazin-1-yl)methyl)-3-(((S)-oxetan-2-yl)methyl)-3H-imidazo[4,5-c]pyridine-6-carbonitrile

To a solution of (S)-1-(6-((4-chloro-2-fluorobenzyl)oxy)-3-fluoropyridin-2-yl)-3-methylpiperazine TFA salt (340 mg, crude) in DMF (5.0 mL) was added DIEA (490 mg, 3.8 mmol). After 2 minutes, (S)-2-(chloromethyl)-3-(oxetan-2-ylmethyl)-3H-imidazo[4,5-c]pyridine-6-carbonitrile (100 mg, 0.38 mmol) was added. The resulting mixture was stirred at 60° C. for 2 h. After the reaction was completed, the mixture was evaporated and the residue purified by column chromatography on silica gel (DCM/MeOH=40/1) to give 2-(((S)-4-(6-((4-chloro-2-fluorobenzyl)oxy)-3-fluoropyridin-2-yl)-2-methylpiperazin-1-yl)methyl)-3-(((S)-oxetan-2-yl)methyl)-3H-imidazo[4,5-c]pyridine-6-carbonitrile (180 mg, yield: 81%) as yellow oil. MS Calcd.: 579.2; MS Found: 580.2 [M+H]⁺.

### Step B: (2S)-4-{6-[(4-chloro-2-fluorophenyl)methoxy]-3-fluoropyridin-2-yl}-2-methyl-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 525a)

(2S)-4-{6-[(4-chloro-2-fluorophenyl)methoxy]-3-fluoropyridin-2-yl}-2-methyl-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperazine (Compound 525a) (30 mg) was obtained as a white solid.

MS Calcd.: 689.2; MS Found: 690.0 [M+H]⁺.

¹H NMR (400 MHz, DMSO-d₆): δ 9.19 (s, 1H), 8.30 (s, 1H), 7.42-7.53 (m, 3H), 7.29 (dd, J=8.4 Hz, 2.0 Hz, 1H), 6.24 (dd, J=8.4 Hz, 2.0 Hz, 1H), 5.17-5.35 (m, 3H), 4.80-4.93 (m, 2H), 4.30-4.55 (m, 3H), 3.53-3.90 (m, 3H), 2.65-3.25 (m, 5H), 2.30-2.52 (m, 2H), 1.00-1.20 (br.s, 3H)¹⁹F NMR (377 MHz, DMSO-d₆): δ −63.73, −115.39, −140.72.

### Example 331: 5-{6-[(4-{6-[(4-chloro-2-fluorophenyl)methoxy]pyridin-2-yl}piperidin-1-yl)methyl]-7-{[(2S)-oxetan-2-yl]methyl}-7H-imidazo[4,5-c]pyridazin-3-yl}-4H-1,2,4-triazole-3-carbonitrile (Compound 526a)

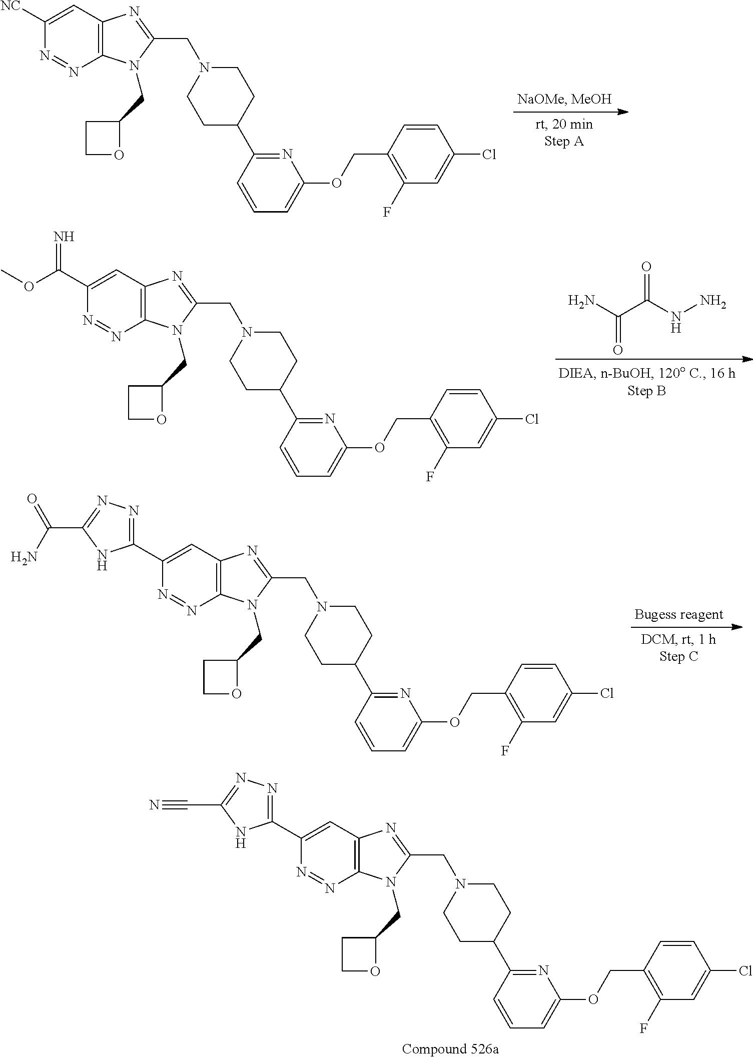 [SMILES: N#Cc1cc2nc(CN3CCC(c4cccc(OCc5ccc(Cl)cc5F)n4)CC3)n(C[C@@H]3CCO3)c2nn1 ; COC(=N)c1cc2nc(CN3CCC(c4cccc(OCc5ccc(Cl)cc5F)n4)CC3)n(C[C@@H]3CCO3)c2nn1 ; NNC(=O)C(N)=O ; NC(=O)c1nnc(-c2cc3nc(CN4CCC(c5cccc(OCc6ccc(Cl)cc6F)n5)CC4)n(C[C@@H]4CCO4)c3nn2)[nH]1 ; N#Cc1nnc(-c2cc3nc(CN4CCC(c5cccc(OCc6ccc(Cl)cc6F)n5)CC4)n(C[C@@H]4CCO4)c3nn2)[nH]1]

### Step A: methyl (S)-6-((4-(6-((4-chloro-2-fluorobenzyl)oxy)pyridin-2-yl)piperidin-1-yl)methyl)-7-(oxetan-2-ylmethyl)-7H-imidazo[4,5-c]pyridazine-3-carbimidate

A mixture of (S)-6-((4-(6-((4-chloro-2-fluorobenzyl)oxy)pyridin-2-yl)piperidin-1-yl)methyl)-7-(oxetan-2-ylmethyl)-7H-imidazo[4,5-c]pyridazine-3-carbonitrile (160 mg, 0.29 mmol), Sodium methoxide (158 mg, 2.9 mmol) in MeOH (3.0 mL) was stirred at room temperature for 20 mins. After the reaction was completed, the mixture was diluted with EA (20 mL), washed with H₂O (10 mL×2). The organic layer was dried over anhydrous sodium sulfate, filtered and evaporated to dryness. The mixture was concentrated in vacuum to give methyl (S)-6-((4-(6-((4-chloro-2-fluorobenzyl)oxy)pyridin-2-yl)piperidin-1-yl)methyl)-7-(oxetan-2-ylmethyl)-7H-imidazo[4,5-c]pyridazine-3-carbimidate (120 mg, yield: 71%) as yellow oil. MS Calcd.: 579.2; MS Found: 580.2 [M+H]⁺.

### Step B: (S)-5-(6-((4-(6-((4-chloro-2-fluorobenzyl)oxy)pyridin-2-yl)piperidin-1-yl)methyl)-7-(oxetan-2-ylmethyl)-7H-imidazo[4,5-c]pyridazin-3-yl)-4H-1,2,4-triazole-3-carboxamide

To a solution of methyl (S)-6-((4-(6-((4-chloro-2-fluorobenzyl)oxy)pyridin-2-yl)piperidin-1-yl)methyl)-7-(oxetan-2-ylmethyl)-7H-imidazo[4,5-c]pyridazine-3-carbimidate (120 mg) in n-BuOH (2 mL) was added DIEA (81 mg, 0.63 mmol) and 2-hydrazineyl-2-oxoacetamide (42 mg, 0.41 mmol) at room temperature. The reaction was stirred at 120° C. for 16 hours. After the reaction was completed, the reaction was concentrated, quenched with water (5 mL) and extracted with ethyl acetate (10 mL×3). The organic layer was combined and washed with brine (5 mL), dried over sodium sulfate, filtered and concentrated in vacuum. The residue was purified by column chromatography on silica gel (MeOH/DCM=20/1) to give (S)-5-(6-((4-(6-((4-chloro-2-fluorobenzyl)oxy)pyridin-2-yl)piperidin-1-yl)methyl)-7-(oxetan-2-ylmethyl)-7H-imidazo[4,5-c]pyridazin-3-yl)-4H-1,2,4-triazole-3-carboxamide (62 mg, yield: 47%) as a yellow solid. MS Calcd.: 632.2; MS Found: 633.2 [M+H]⁺.

### Step C: 5-{6-[(4-{6-[(4-chloro-2-fluorophenyl)methoxy]pyridin-2-yl}piperidin-1-yl)methyl]-7-{[(2S)-oxetan-2-yl]methyl}-7H-imidazo[4,5-c]pyridazin-3-yl}-4H-1,2,4-triazole-3-carbonitrile (Compound 526a)

To a mixture of (S)-5-(6-((4-(6-((4-chloro-2-fluorobenzyl)oxy)pyridin-2-yl)piperidin-1-yl)methyl)-7-(oxetan-2-ylmethyl)-7H-imidazo[4,5-c]pyridazin-3-yl)-4H-1,2,4-triazole-3-carboxamide (62 mg, 0.098 mmol), Burgess reagent (93.48 mg, 0.39 mmol) in DCM (2.0 mL) was stirred at rt for 1 hour. After the reaction was completed, the mixture was diluted with EA (20 mL), washed with H₂O (10 mL×2). The organic layer was dried over anhydrous sodium sulfate, filtered and evaporated to dryness. The organic layer was concentrated and purified by Prep-HPLC (0.1% FA/H₂O/CH₃CN) to give 5-{6-[(4-{6-[(4-chloro-2-fluorophenyl)methoxy]pyridin-2-yl}piperidin-1-yl)methyl]-7-{[(2S)-oxetan-2-yl]methyl}-7H-imidazo[4,5-c]pyridazin-3-yl}-4H-1,2,4-triazole-3-carbonitrile (Compound 526a) (15 mg, yield: 26%) as a white solid. MS Calcd.: 614.2; MS Found: 615.1 [M+H]⁺.

¹H NMR (400 MHz, DMSO-d₆): δ 8.51 (s, 1H), 7.64 (t, J=8.0 Hz, 1H), 7.57 (t, J=8.0 Hz, 1H), 7.46 (dd, J=10.0 Hz, 2.0 Hz, 1H), 7.30 (dd, J=8.0 Hz, 1.2 Hz, 1H), 6.89 (d, J=7.6 Hz, 1H), 6.68 (d, J=8.0 Hz, 1H), 5.38 (s, 2H), 5.26-5.33 (m, 1H), 5.00-5.08 (m, 1H), 4.87-4.95 (m, 1H), 4.48-4.55 (m, 1H), 4.38-4.48 (m, 1H), 4.02-4.11 (m, 2H), 2.95-3.08 (m, 2H), 2.71-2.81 (m, 1H), 2.50-2.70 (m, 2H), 2.26-2.38 (m, 2H), 1.70-1.88 (m, 4H). ¹⁹F NMR (377 MHz, DMSO-d₆): δ −115.22.

### Example 332: 4-[(4-chloro-2-fluorophenyl)methoxy]-5-fluoro-2-[(3S)-3-methyl-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazin-1-yl]pyrimidine (Compound 527a)

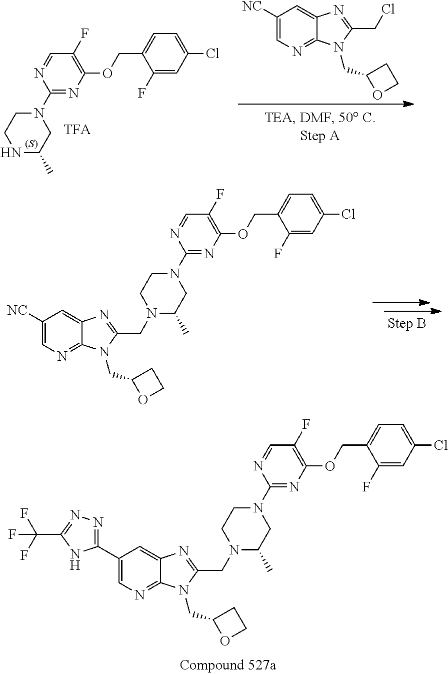

### Step A: 2-(((S)-4-(4-((4-chloro-2-fluorobenzyl)oxy)-5-fluoropyrimidin-2-yl)-2-methylpiperazin-1-yl)methyl)-3-(((S)-oxetan-2-yl)methyl)-3H-imidazo[4,5-b]pyridine-6-carbonitrile

A mixture of (S)-4-((4-chloro-2-fluorobenzyl)oxy)-5-fluoro-2-(3-methylpiperazin-1-yl)pyrimidine TFA salt (357 mg, crude), (S)-2-(chloromethyl)-3-(oxetan-2-ylmethyl)-3H-imidazo[4,5-b]pyridine-6-carbonitrile (200 mg, 0.76 mmol), TEA (231 mg, 2.3 mmol) in DMF (3 mL) was stirred at 50° C. for 2 hours. After the reaction was completed, the mixture was concentrated and the residue was purified by column chromatography on silica gel (DCM/MeOH=95/5) to give 2-(((S)-4-(4-((4-chloro-2-fluorobenzyl)oxy)-5-fluoropyrimidin-2-yl)-2-methylpiperazin-1-yl)methyl)-3-(((S)-oxetan-2-yl)methyl)-3H-imidazo[4,5-b]pyridine-6-carbonitrile (330 mg, yield: 75%) as a yellow oil. MS Calcd.: 580.2; MS Found: 580.9 [M+H]⁺.

### Step B: 4-[(4-chloro-2-fluorophenyl)methoxy]-5-fluoro-2-[(3S)-3-methyl-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazin-1-yl]pyrimidine (Compound 527a)

4-[(4-chloro-2-fluorophenyl)methoxy]-5-fluoro-2-[(3S)-3-methyl-4-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperazin-1-yl]pyrimidine (Compound 527a) (29 mg, yield: 17%) was obtained as a white solid.

MS Calcd.: 690.2; MS Found: 691.3 [M+H]⁺.

¹H NMR (400 MHz, DMSO-d₆): δ 8.99 (d, J=2.0 Hz, 1H), 8.56 (d, J=1.6 Hz, 1H), 8.20 (d, J=2.8 Hz, 1H), 7.58 (t, J=8.4 Hz, 1H), 7.50 (dd, J=10.0 Hz, J=2.0 Hz, 1H), 7.33 (dd, J=8.0 Hz, J=1.6 Hz, 1H), 5.47 (s, 2H), 5.20-5.28 (m, 1H), 4.73-4.85 (m, 2H), 4.42-4.54 (m, 2H), 4.18-4.26 (m, 1H), 4.10-4.18 (m, 1H), 4.01-4.09 (m, 1H), 3.68 (d, J=14.0 Hz, 1H), 3.15-3.22 (m, 1H), 2.99-3.08 (m, 1H), 2.55-2.75 (m, 3H), 2.36-2.51 (m, 1H), 2.28-2.36 (m, 1H), 1.14 (d, J=6.4 Hz, 3H). ¹⁹F NMR (377 MHz, DMSO-d₆): δ −62.94, −114.82, −171.97.

### Example A: cAMP Assays

Activation of GLP-1 receptor is known to stimulate cyclic AMP (cAMP) production in cells which indicates primary coupling to the G_{αs} subunit of the G protein heterotrimeric complex. Evidence suggests signaling through G_{αs} induced cAMP stimulation elicits the desired pharmacological response regarding insulin release from pancreatic β-cells.

To optimize functional activity directed toward G_{αs} coupling, a HEK293/CRE-Luc cell line developed by HDB stably expressing the GLP-1 Receptor was used. 200×concentration of compound working solutions were prepared (Agilent Technologies Bravo) with 1/2 log serial dilution in 384-well Echo LDV plate (Labcyte, Cat #LP-0200). 50 nL/well 200× concentration of compound working solutions were moved to 384-well white low volume plate (Greiner,Cat #784075) using Labcyte ECHO550. 1×10⁵ cells/mL HEK293/GLP1R/CRE-LUC(HD Biosciences) cell suspensions prepared with assay buffer[DPBS containing 0.5 mM IBMX(Sigma, Cat #15879) and 0.1% BSA (GENVIEW, Cat #FA016-100 g)], 10 uL cell suspensions were added to each well of previous generated assay plate which already contains 50 nl compound at 200×concentration using ThermoFisher Multidrop Combi (1000 cells/well). Seal the plate and incubate at 37° C. with 5% CO2 for 30 min.

After incubation the cAMP assay signal was generated using cAMP dynamic 2 Kit (Cisbio). 5 μL cAMP-d2 working solution was added to each well, followed with 5 μL Anti-cAMP antibody-cryptate working solution added to each well using ThermoFisher Multidrop Combi. Incubate at room temperature for 1 hour protected from light. Read the fluorescence at 665 and 615 nm with Reader PerkinElmer EnVision.

% Activity=100%×(mean RLU of test sample−mean RLU of vehicle control)/(mean RLU of MAX control−mean RLU of vehicle control))

Table 1 shows the biological activity of compounds in GLP-1R agonist cAMP stimulation assay (EC₅₀)

| Compound No. | EC₅₀ (nM) |
|---|---|
| 101a | 1.09 |
| 102a | 0.539 |
| 103a | 0.309 |
| 104a | 0.207 |
| 105a | 0.175 |
| 106a | 1.19 |
| 107a | 0.337 |
| 108a | 155 |
| 109a | 13.2 |
| 110a | 15.9 |
| 111a | 1.09 |
| 112a | 6.35 |
| 113a | 38.2 |
| 114a | 58.9 |
| 115a | 1.17 |
| 129a | 0.56 |
| 130a | 5.1 |
| 150a | 0.61 |
| 151a | 0.8 |
| 152a | 0.88 |
| 153a | 0.3 |
| 154a | 0.13 |
| 154b | 0.074 |
| 154c | 1.5 |
| 155a | 1 |
| 156a | 3 |
| 166a | 0.14 |
| 167a | 1.1 |
| 168a | 0.058 |
| 169a | 0.92 |
| 170a | 0.14 |
| 171a | 0.27 |
| 172a | 0.31 |
| 173a | 0.26 |
| 174a | 0.56 |
| 175a | 1.5 |
| 176a | 8.6 |
| 177a | 5.9 |
| 178a | 0.57 |
| 179a | 0.27 |
| 180a | 6 |
| 181a | 0.31 |
| 182a | 2.5 |
| 183a | 4 |
| 184a | 2 |
| 185a | 5.3 |
| 186a | 3 |
| 187a | 0.88 |
| 188a | 0.043 |
| 189a | 0.82 |
| 190a | 1.2 |
| 191a | 8.8 |
| 192a | 6.2 |
| 193a | 0.23 |
| 194a | 0.47 |
| 195a | 0.63 |
| 196a | 1.1 |
| 197a | 4.3 |
| 198a | 12 |
| 199a | 7.4 |
| 200a | 0.35 |
| 201a | 3.1 |
| 202a | 6 |
| 203a | 3.2 |
| 131a | 6.3 |
| 135a | 0.64 |
| 204a | 0.84 |
| 118a | 10 |
| 205a | 2.8 |
| 206a | 0.78 |
| 207a | 0.39 |
| 145a | 0.16 |
| 146a | 0.49 |
| 119a | 7.2 |
| 120a | 4 |
| 122a | 2.4 |
| 123a | 4 |
| 124a | 0.78 |
| 125a | 0.056 |
| 208a | 1.8 |
| 209a | 3.2 |
| 210a | 4.2 |
| 211a | 3.6 |
| 212a | 7.5 |
| 149a | 4.8 |
| 213a | 0.83 |
| 214a | 4.2 |
| 215a | 2.7 |
| 216a | 8.8 |
| 217a | 4.6 |
| 218a | 0.013 |
| 219a | 1.6 |
| 161a | 0.51 |
| 220a | 2.8 |
| 221a | 7.4 |
| 222a | 0.55 |
| 223a | 0.12 |
| 224a | 0.018 |
| 224b | 1.7 |
| 224c | 0.014 |
| 225a | 0.064 |
| 226a | 0.13 |
| 227a | 0.094 |
| 228a | 0.19 |
| 229a | 0.046 |
| 229b | 8.9 |
| 229c | 0.04 |
| 230a | 0.88 |
| 231a | 0.031 |
| 232a | 0.16 |
| 233a | 4 |
| 234a | 3.7 |
| 235a | 0.062 |
| 236a | 2 |
| 237a | 9.1 |
| 238a | 0.12 |
| 239a | 0.071 |
| 239b | 0.04 |
| 239c | 0.55 |
| 240a | 1.1 |
| 241a | 0.32 |
| 242a | 0.36 |
| 243a | 0.052 |
| 244a | 0.14 |
| 245a | 2.6 |
| 246a | 0.23 |
| 247a | 3.4 |
| 248a | 0.11 |
| 249a | 0.27 |
| 250a | 0.11 |
| 251a | 0.11 |
| 252a | 0.066 |
| 253a | 0.65 |
| 254a | 0.069 |
| 255a | 0.24 |
| 256a | 0.23 |
| 257a | 0.098 |
| 258a | 0.16 |
| 259a | 0.074 |
| 260a | 0.031 |
| 261a | 0.018 |
| 396a | 3.2 |
| 397a | 1.3 |
| 247b | 0.093 |
| 398a | 4.3 |
| 334a | 0.25 |
| 399a | 17 |
| 400a | 3.7 |
| 401a | 0.6 |
| 402 | 0.75 |
| 262 | 0.071 |
| 403a | 0.02 |
| 332a | 1.6 |
| 333a | 0.03 |
| 333b | 0.029 |
| 333c | 0.95 |
| 404a | 0.045 |
| 353a | 0.026 |
| 354a | 0.11 |
| 405a | 0.53 |
| 355a | 7 |
| 406a | 0.018 |
| 293a | 2.8 |
| 294a | 0.47 |
| 295a | 2.6 |
| 296a | 0.48 |
| 320a | 6.8 |
| 318a | 10 |
| 319a | 0.36 |
| 324a | 0.11 |
| 322a | 0.078 |
| 323a | 0.016 |
| 321a | 0.022 |
| 327a | 0.013 |
| 325a | 0.012 |
| 375a | 0.0063 |
| 366a | 0.012 |
| 360a | 0.0092 |
| 289a | 0.0051 |
| 307a | 0.26 |
| 308a | 0.5 |
| 316a | 0.037 |
| 344a | 0.022 |
| 343a | 0.079 |
| 361a | 0.034 |
| 347a | 0.022 |
| 328a | 0.37 |
| 330a | 0.97 |
| 345a | 0.014 |
| 346a | 0.015 |
| 299a | 0.26 |
| 297a | 0.098 |
| 281a | 0.023 |
| 266a | 0.094 |
| 267a | 0.051 |
| 393a | 0.063 |
| 390a | 0.48 |
| 407a | 4.5 |
| 408a | 0.28 |
| 409a | 6.5 |
| 410a | 0.029 |
| 410b | 0.082 |
| 283a | 0.1 |
| 412a | 0.033 |
| 413 | 0.6 |
| 414 | 0.055 |
| 415 | 0.14 |
| 270a | 0.86 |
| 271a | 0.34 |
| 277a | 0.15 |
| 268a | 4.2 |
| 416a | 14 |
| 276a | 0.56 |
| 282a | 0.45 |
| 292a | 0.1 |
| 269a | 2 |
| 317a | 0.11 |
| 329a | 0.25 |
| 417a | 0.0065 |
| 418a | 0.095 |
| 419a | 0.072 |
| 420a | 0.21 |
| 421a | 0.34 |
| 422a | 0.17 |
| 423a | 0.13 |
| 424a | 0.13 |
| 425a | 0.13 |
| 426a | 0.088 |
| 288a | 0.0087 |
| 341a | 0.026 |
| 342a | 0.0064 |
| 359a | 0.014 |
| 273a | 0.019 |
| 427a | 0.76 |
| 428a | 0.36 |
| 429a | 0.24 |
| 430a | 0.04 |
| 431a | 0.11 |
| 432a | 6.9 |
| 433a | 0.013 |
| 302a | 0.17 |
| 434a | 0.23 |
| 435a | 0.018 |
| 436a | 0.91 |
| 437a | 2.7 |
| 438a | 0.026 |
| 439a | 2.3 |
| 440a | 0.04 |
| 441a | 0.025 |
| 442a | 0.033 |
| 443a | 0.28 |
| 444a | 0.0037 |
| 445a | 2 |
| 446 | 0.12 |
| 300a | 0.1 |
| 348a | 0.062 |
| 352a | 0.43 |
| 447a | 0.019 |
| 280a | 0.11 |
| 448a | 0.011 |
| 449a | 0.016 |
| 450a | 0.062 |
| 451a | 0.21 |
| 452a | 0.14 |
| 453a | 1.9 |
| 454a | 0.17 |
| 455a | 0.12 |
| 456a | 0.085 |
| 457a | 0.12 |
| 458a | 0.3 |
| 459a | 0.0088 |
| 460a | 0.15 |
| 461a | 0.23 |
| 462a | 0.045 |
| 463a | 0.35 |
| 464a | 0.76 |
| 465a | 0.3 |
| 466a | 0.19 |
| 467a | 0.076 |
| 313a | 0.012 |
| 468a | 0.53 |
| 469a | 0.087 |
| 470a | 0.22 |
| 471a | 0.11 |
| 472a | 0.012 |
| 473a | 0.46 |
| 474a | 1.5 |
| 475a | 0.41 |
| 476a | 0.25 |
| 477a | 0.98 |
| 478a | 0.1 |
| 479a | 0.1 |
| 480a | 0.018 |
| 481a | 0.0048 |
| 482a | 0.032 |
| 483a | 0.51 |
| 484a | 0.0063 |
| 485a | 0.0096 |
| 486a | 0.038 |
| 487a | 0.029 |
| 488a | 0.054 |
| 489a | 0.087 |
| 490a | 0.086 |
| 491a | 0.057 |
| 492a | 0.084 |
| 493a | 0.044 |
| 494a | 0.013 |
| 495a | 0.0084 |
| 496a | 0.017 |
| 497a | 0.034 |
| 498a | 0.099 |
| 499a | 0.04 |
| 500a | 0.078 |
| 501a | 0.038 |
| 502a | 0.092 |
| 503a | 0.078 |
| 504a | 0.092 |
| 505a | 0.045 |
| 506a | 0.076 |
| 507a | 0.068 |
| 508a | 0.42 |
| 509a | 0.074 |
| 510a | 0.16 |
| 511a | 0.079 |
| 512a | 0.14 |
| 513a | 0.064 |
| 514a | 0.071 |
| 515a | 0.057 |
| 516a | 0.018 |
| 517a | 0.043 |
| 518a | 0.41 |
| 519a | 0.04 |
| 520a | 0.34 |
| 521a | 0.51 |
| 522a | 0.46 |
| 523a | 0.14 |
| 524a | 0.45 |
| 525a | 0.026 |
| 526a | 0.017 |
| 527a | 0.033 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

LENGTHY TABLES
The patent application contains a lengthy table section. A copy of the table is available in electronic form from the USPTO web site (https://seqdata.uspto.gov/?pageRequest=docDetail&DocID=US20230391760A1). An electronic copy of the table will also be available from the USPTO upon request and payment of the fee set forth in 37 CFR 1.19(b)(3).

What is claimed is:

1. A compound of Formula I: 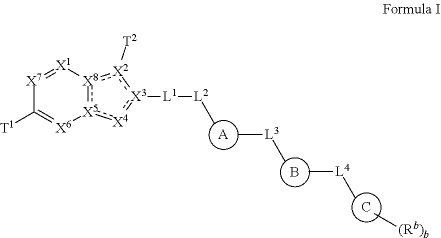
or a pharmaceutically acceptable salt or solvate thereof, wherein: [symbol]
indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
—N(R^{s})C(═O)R^{z};
—N(R^{s})C(═O)OR^{z};
—N(R^{s})C(═O)N(R^{s})R^{z};
—N(R^{s})S(O)₁₋₂—R^{z};
—N(R^{s})S(═NR^{s})(═O)R^{z};
—S(O)₁₋₂R^{z};
—P(═O)R^{z1}R^{z2};
—C(═O)OH;
—C(═O)N(R^{s})R^{z};
—S(O)₁₋₂N(R^{s})R^{z};
—S(═NR^{s})(═O)N(R^{s})R^{z};
5- to 10-membered heteroaryl optionally substituted with 1-4 R^{v}, and wherein the heteroaryl optionally comprises an endocyclic group selected from the group consisting of: 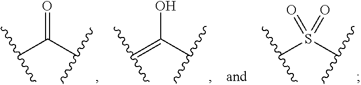
5- to 10-membered heterocycloalkyl, wherein the heterocycloalkyl comprises an endocyclic group selected from the group consisting of: 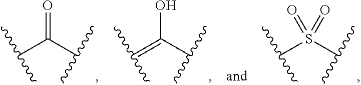
wherein the heterocycloalkyl is optionally substituted with 1-4 R^{v}; and
(C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring; or when q is 2 or 3, a pair of R^{x} on adjacent carbon atoms, taken together form a double bond between the adjacent carbon atoms;
R^{z}, R^{z1}, and R^{z2} are each independently selected from the group consisting of:
hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 10-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, (C₃-C₈)cycloalkyl, —O(C₁-C₆)alkylene-O(C₁-C₆)alkyl, —OH, —N(R^{s})₂ 3- to 8-membered heterocycloalkyl, —CO₂H, (C₁-C₆)hydroxyalkyl, (C₁-C₆)alkyl-S(O)₁₋₂—(C₁-C₆)alkyl, (C₁-C₆)alkyl-C(═O)OH, —S(O)₀₋₂—C₁-C₆ alkyl, (C₁-C₆)alkenyl, —P(═O)R^{z1}R^{z2}, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T};
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, —C(═O)C₁-C₆ alkyl, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of: 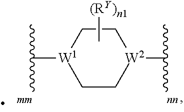
wherein n1 is 0, 1, or 2; W¹ is CR^{Y1} or N; and W² is CR^{Y2} or N; 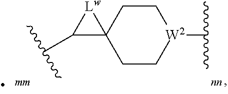
wherein W² is CR^{Y2} or N, L^{w} is (C₁-C₃)alkylene;
phenylene optionally substituted with 1-4 R^{Y};
5- to 6-membered heteroarylene optionally substituted with 1-3 R^{Y};
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y};
wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is a bond;
Ring B is selected from the group consisting of: (B-I), (B-II), (B-III), (B-IV), and (B-V): 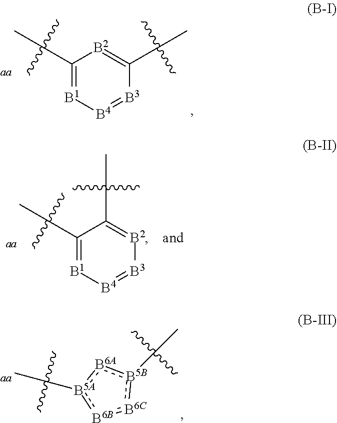
wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl; 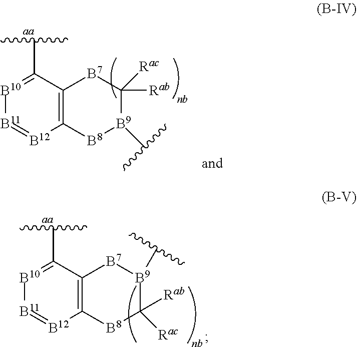
wherein aa represents the point of attachment to L³;
B⁷ and B⁸ are independently selected from the group consisting of: —O—, —NR^{N}—, and —C(R¹)₂—;
B is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, CN, —C(O)NH₂, —CONH(C₁-C₃ alkyl) and C(O)N(C₁-C₃)alky)₂; and
b is an integer selected from 0-3.

2. The compound of claim 1, wherein T¹ is -T³.

3. The compound of claim 1, wherein T¹ is -L^{a}-(CR^{x}R^{x})_{q}-T³.

4. The compound of claim 1 or 3, wherein -L^{a} is a bond.

5. The compound of any one of claim 1 or 3-4, wherein -L^{a} is —NH—, —N(C₁₋₃ alkyl)-, —O—, or —S—.

6. The compound of any one of claim 1 or 3-5, wherein -L^{a} is —O—.

7. The compound of any one of claim 1 or 3-6, wherein q is 1.

8. The compound of any one of claim 1 or 3-6, wherein q is 2 or 3.

9. The compound of any one of claim 1 or 3-8, wherein each R^{x} is hydrogen or (C₁-C₆)alkyl.

10. The compound of any one of claim 1 or 3-9, wherein each R^{x} is hydrogen.

11. The compound of any one of claim 1 or 3-8, wherein one pair of R^{x} on the same carbon, taken together with the carbon atom to which each is attached, forms a (C₃-C₈)cycloalkyl ring.

12. The compound of any one of claims 1, 3-8, or 11, wherein one pair of R^{x} on the same carbon, taken together with the carbon atom to which each is attached, forms a cyclopropyl.

13. The compound of claim 11 or 12, wherein each remaining R^{x} is hydrogen.

14. The compound of claim 1 or 3, wherein T¹ is —(CR^{x}R^{x})-T³.

15. The compound of claim 14, wherein each R^{x} is hydrogen.

16. The compound of claim 14, wherein the pair of R^{x} taken together with the carbon atom to which each is attached, forms a (C₃-C₈)cycloalkyl ring.

17. The compound of claim 1 or 3, wherein T¹ is —(CR^{x}R^{x})_{q}-T³; and q is 2 or 3.

18. The compound of claim 17, wherein each R^{x} is hydrogen.

19. The compound of claim 1 or 3, wherein T¹ is —O—(CR^{x}R^{x})_{q}-T³; and q is 1, 2, or 3.

20. The compound of claim 19, wherein q is 1.

21. The compound of claim 19 or 20, wherein each R^{x} is hydrogen.

22. The compound of any one of claims 1-21, wherein T³ is selected from the group consisting of: —N(R^{s})C(═O)R^{z}, —N(R^{s})C(═O)OR^{z}, —N(R^{s})C(═O)N(R^{s})R^{z}, —N(R^{s})S(O)₁₋₂—R^{z}, and —N(R^{s})S(═NR^{s})(═O)R^{z}.

23. The compound of any one of claims 1-22, wherein T³ is —N(R^{s})C(═O)R^{z}.

24. The compound of any one of claims 1-23, wherein T³ is —NHC(═O)R^{z}.

25. The compound of any one of claims 1-22, wherein T³ is —N(R^{s})S(O)₁₋₂—R^{z}.

26. The compound of any one of claims 1-22 or 25, wherein T³ is —NHS(O)₂R^{z}.

27. The compound of any one of claims 1-21, wherein T³ is —S(O)₁₋₂R^{z}.

28. The compound of any one of claims 1-21 or 27, wherein T³ is —S(O)₂R^{z}.

29. The compound of any one of claims 1-28, wherein R^{z} is (C₁-C₆)alkyl optionally substituted with 1-4 R^{v}.

30. The compound of any one of claims 1-29, wherein R^{z} is (C₁-C₃)alkyl optionally substituted with 1-4 R^{v}.

31. The compound of any one of claims 1-30, wherein R^{z} is (C₁-C₃)alkyl.

32. The compound of any one of claims 1-31, wherein R^{z} is methyl, ethyl, or isopropyl.

33. The compound of any one of claims 1-30, wherein R^{z} is (C₁-C₃)alkyl substituted with 1-3 R^{v}.

34. The compound of any one of claims 1-30 or 33, wherein R^{z} is (C₁-C₃)alkyl substituted with from 1-3 substituents each independently selected from the group consisting of halo and (C₁-C₃)alkoxy, such as wherein R^{z} is —CH₂CF₃ or —CH₂CH₂OMe.

35. The compound of any one of claims 1-28, wherein R^{z} is —R^{z3} or -L^{b}-R^{z3}.

36. The compound of any one of claims 1-28 or 35, wherein R^{z} is —R^{z3} or —CH₂—R^{z3}.

37. The compound of any one of claims 1-28 or 35-36, wherein R^{z3} is selected from the group consisting of: (C₃-C₆)cycloalkyl and 3- to 6-membered heterocycloalkyl, each of which is optionally substituted with 1-4 R^{v}.

38. The compound of any one of claims 1-28 or 35-37, wherein R^{z3} is selected from the group consisting of: (C₃-C₆)cycloalkyl and 3- to 6-membered heterocycloalkyl, each optionally substituted with 1-2 R^{v}.

39. The compound of any one of claims 1-28 or 35-38, wherein R^{z3} is cyclopropyl, cyclobutyl, difluorocyclobutyl, cyclopentyl, oxetanyl, tetrahydropyranyl, or tetrahydro-2H-thiopyran 1,1-dioxide.

40. The compound of any one of claims 1-28 or 35-36, wherein R^{z3} is selected from the group consisting of: (C₆-C₁₀)aryl and 5- to 10-membered heteroaryl, each of which is optionally substituted with 1-4 R^{v}.

41. The compound of any one of claims 1-28, 35-36, or 40, wherein R^{z3} is phenyl optionally substituted with 1-2 R^{v}.

42. The compound of any one of claims 1-21, wherein T³ is —NHC(═O)R^{z} or —NHS(O)₁₋₂—R^{z}; and R^{z} is (C₁-C₆)alkyl optionally substituted with 1-4 R^{v}.

43. The compound of claim 40, wherein R^{z} is (C₁-C₃)alkyl optionally substituted with (C₁-C₃)alkoxy; or wherein R^{z} is (C₁-C₃)alkyl substituted with 1-3 halo.

44. The compound of any one of claims 1-21, wherein T³ is —NHC(═O)R^{z} or —NHS(O)₁₋₂—R^{z}; and R^{z} is —R^{z3} or —CH₂—R^{z3}.

45. The compound of claim 42, wherein R^{z3} is selected from the group consisting of: phenyl, (C₃-C₆)cycloalkyl, and 3- to 6-membered heterocycloalkyl, each optionally substituted with 1-2 R^{v}, such as wherein R^{z3} is phenyl, cyclopropyl, cyclobutyl, difluorocyclobutyl, cyclopentyl, oxetanyl, tetrahydropyranyl, or tetrahydro-2H-thiopyran 1,1-dioxide.

46. The compound of any one of claims 1-21, wherein T³ is —S(O)₂R^{z}; and R^{z} is (C₁-C₆)alkyl optionally substituted with 1-4 R^{v}.

47. The compound of any one of claims 1-21, wherein T³ is —C(═O)OH.

48. The compound of any one of claims 1-21, wherein T³ is —P(═O)R^{z1}R^{z2}.

49. The compound of any one of claims 1-21 or 48, wherein R^{z1} and R^{z2} are each independently selected (C₁-C₃)alkyl.

50. The compound of any one of claims 1-21, wherein T³ is 5- to 10-membered heteroaryl optionally substituted with 1-4 R^{v}, and wherein the heteroaryl optionally comprises an endocyclic group selected from the group consisting of:

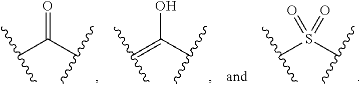

51. The compound of any one of claims 1-21 or 50, wherein T³ is 5- to 6-membered heteroaryl optionally substituted with 1-4 R^{v}.

52. The compound of any one of claims 1-21 or 50-51, wherein T³ is 5-membered heteroaryl having 2-4 ring heteroatoms selected from the group consisting of N, O, and S, wherein the heteroaryl is optionally substituted with 1-2 R^{v}.

53. The compound of any one of claims 1-21 or 50-52, wherein T³ is selected from the group consisting of:

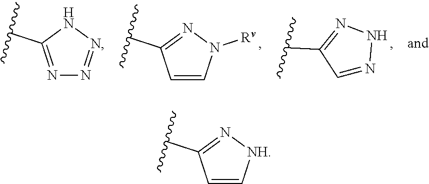

54. The compound of any one of claims 1-21 or 50, wherein T³ is 5- to 6-membered heteroaryl that comprises an endocyclic group selected from the group consisting of: 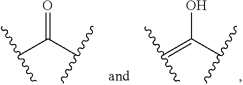
wherein the heteroaryl is further optionally substituted with 1-4 R^{v}.

55. The compound of any one of claims 1-21, 50, or 54, wherein T³ is selected from the group consisting of:

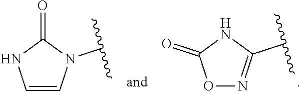 [SMILES: *n1cc[nH]c1=O ; *c1noc(=O)[nH]1]

56. The compound of any one of claims 1-21, wherein T³ is 5- to 10-membered heterocycloalkyl, wherein the heterocycloalkyl comprises an endocyclic group selected from the group consisting of: 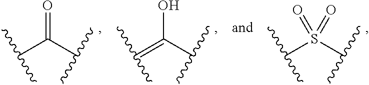
wherein the heterocycloalkyl is optionally substituted with 1-4 R^{v}.

57. The compound of any one of claims 1-21 or 56, wherein T³ is 5- to 6-membered heterocycloalkyl which comprises an endocyclic group selected from the group consisting of: 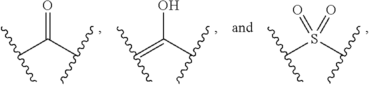
wherein the heterocycloalkyl is optionally substituted with 1-4 R^{v}.

58. The compound of any one of claims 1-21 or 56-57, wherein T³ is 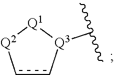
Q¹ is C(═O) or S(O)₂; Q² is O, NH, —CH₂—, or —CH₂—CH₂—; Q³ is N or CH; and [symbol] is a single bond or a double bond, provided that T³ is non-aromatic.

59. The compound of any one of claims 1-21 or 56-58, wherein T³ is selected from the group consisting of:

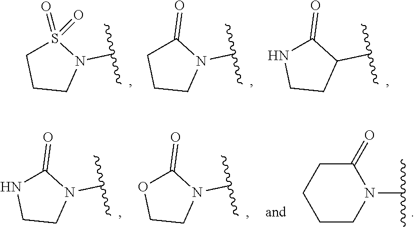

60. The compound of claim 1, wherein T is —N(R^{s})C(═O)R^{z} or —N(R^{s})S(O)₁₋₂—R^{z}; and R^{z} is (C₁-C₆)alkyl optionally substituted with 1-4 R^{v}.

61. The compound of claim 60, wherein R^{z} is (C₁-C₃)alkyl.

62. The compound of claim 60, wherein R^{z} is (C₁-C₃)alkyl substituted with from 1-3 substituents each independently selected from the group consisting of halo and (C₁-C₃)alkoxy, such as wherein R^{z} is —CH₂CF₃ or —CH₂CH₂OMe.

63. The compound of claim 1, wherein T is —N(R^{s})C(═O)R^{z} or —N(R^{s})S(O)₁₋₂—R^{z}; and R^{z} is —R^{z3} or —CH₂—R^{z3}.

64. The compound of claim 63, wherein R^{z3} is selected from the group consisting of: phenyl, (C₃-C₆)cycloalkyl, and 3- to 6-membered heterocycloalkyl, each optionally substituted with 1-2 R^{v}, such as wherein R^{z3} is phenyl, cyclopropyl, cyclobutyl, difluorocyclobutyl, cyclopentyl, oxetanyl, tetrahydropyranyl, or tetrahydro-2H-thiopyran 1,1-dioxide.

65. The compound of any one of claims 60-64, wherein T¹ is —NHC(═O)R^{z}.

66. The compound of any one of claims 60-64, wherein T¹ is —NHS(O)₂—R^{z}.

67. The compound of claim 1, wherein T¹ is —(CR^{x}R^{x})_{q}—S(O)₂R^{z}; and q is 1, 2, or 3.

68. The compound of claim 67, wherein R^{z} is (C₁-C₆)alkyl optionally substituted with 1-4 R^{v}.

69. The compound of claim 67 or 68, wherein R^{z} is (C₁-C₃)alkyl.

70. The compound of any one of claims 67-69, wherein q is 1.

71. The compound of any one of claims 67-70, wherein each R^{x} is hydrogen.

72. The compound of claim 1, wherein T is —(CR^{x}R^{x})_{q}—C(═O)OH or —C(═O)OH; and q is 1, 2, or 3.

73. The compound of claim 72, wherein q is 1.

74. The compound of claim 72, wherein q is 2.

75. The compound of any one of claims 72-74, wherein each R^{x} is hydrogen.

76. The compound of any one of claims 72-74, wherein one pair of R^{x} on the same carbon, taken together with the carbon atom to which each is attached, forms a (C₃-C₈)cycloalkyl ring; and each remaining R^{x} when present is hydrogen.

77. The compound of claim 76, wherein the pair of R^{x} on the same carbon, taken together with the carbon atom to which each is attached, forms a cyclopropyl ring.

78. The compound of claim 1 or 72, wherein T¹ is C(═O)OH,

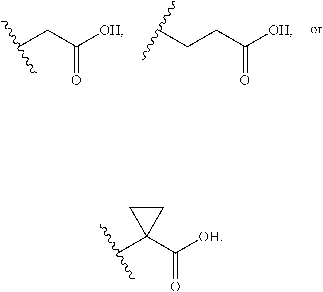 [SMILES: *CC(=O)O ; *CCC(=O)O ; "or" ; *C1(C(=O)O)CC1]

79. The compound of claim 1, wherein T¹ is —O—(CR^{x}R^{x})_{q}—C(═O)OH; and q is 1, 2, or 3.

80. The compound of claim 79, wherein q is 1.

81. The compound of claim 79 or 80, wherein each R^{x} is hydrogen.

82. The compound of claim 1, wherein T¹ is —P(═O)R^{z1}R^{z2}.

83. The compound of claim 82, wherein R^{z1} and R^{z2} are independently selected (C₁-C₃)alkyl.

84. The compound of claim 1, wherein T¹ is 5-membered heteroaryl having 2-4 ring heteroatoms selected from the group consisting of N, O, and S, wherein the heteroaryl is optionally substituted with 1-2 R^{v}.

85. The compound of claim 1 or 84, wherein T¹ is selected from the group consisting of:

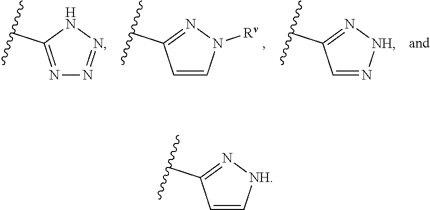

86. The compound of claim 1, wherein T¹ is 5- to 6-membered heteroaryl that comprises an endocyclic group selected from the group consisting of: 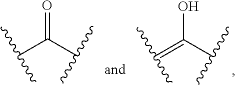
wherein the heteroaryl is further optionally substituted with 1-4 R^{v}.

87. The compound of claim 1 or 86, wherein T¹ is selected from the group consisting of:

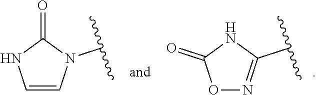 [SMILES: *n1cc[nH]c1=O ; *c1noc(=O)[nH]1]

88. The compound of claim 1, wherein T¹ is; 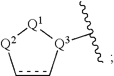
Q¹ is C(═O) or S(O)₂; Q² is O, NH, —CH₂—, or —CH₂—CH₂—; Q³ is N or CH; and [symbol] is a single bond or a double bond, provided that T¹ is non-aromatic.

89. The compound of claim 1 or 88, wherein T¹ is selected from the group consisting of:

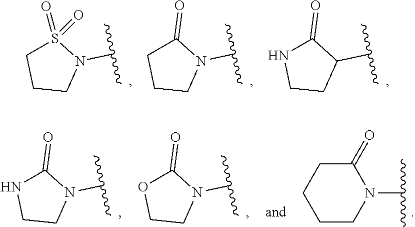

90. The compound of any one of claims 1-89, wherein X² is N; and X⁴ is N.

91. The compound of any one of claims 1-90, wherein X⁸ is C; and X⁵ is C.

92. The compound of any one of claims 1-91, wherein X³ is C.

93. The compound of any one of claims 1-92, wherein X² is N; X³ is C; X⁴ is N; X⁵ is C; and X⁸ is C.

94. The compound of any one of claims 1-93, wherein each of X¹, X⁷, and X⁶ is independently CH or CR^{w}.

95. The compound of any one of claims 1-94, wherein each of X¹, X⁷, and X⁶ is CH.

96. The compound of any one of claims 1-94, wherein one of X¹, X⁷, and X⁶ is CR^{w}; and each remaining of X¹, X⁷, and X⁶ is CH.

97. The compound of any one of claims 1-94 or 96, wherein X⁶ is CR^{w}; and X¹ and X⁷ are CH.

98. The compound of claim 97, wherein X⁶ is C—F.

99. The compound of any one of claims 1-93, wherein one of X¹, X⁷, and X⁶ is N; and each remaining of X¹, X⁷, and X⁶ is CH or CR^{w}.

100. The compound of any one of claims 1-93 or 99, wherein X¹ is N; and X⁶ and X⁷ are CH.

101. The compound of any one of claims 1-93 or 99, wherein X⁶ is N; and X¹ and X⁷ are CH.

102. The compound of any one of claims 1-93 or 99, wherein X⁷ is N; and X¹ and X⁶ are CH.

103. The compound of any one of claims 1-89, wherein the 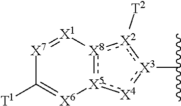
moiety is

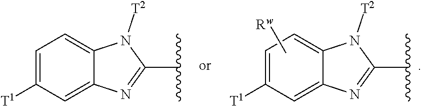

104. The compound of any one of claims 1-89, wherein the

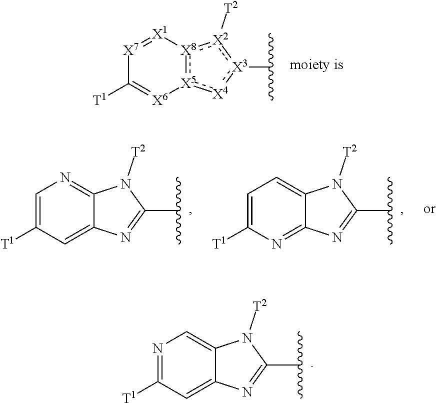

105. The compound of any one of claims 1-104, wherein T² is (C₁-C₆)alkyl which is substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.

106. The compound of any one of claims 1-105, wherein T² is (C₁-C₆)alkyl which is substituted with 3- to 6-membered heterocycloalkyl.

107. The compound of any one of claims 1-106, wherein T² is (C₁-C₃)alkyl which is substituted with 4- to 6-membered heterocycloalkyl.

108. The compound of any one of claims 1-107, wherein T² is (C₁-C₃)alkyl which is substituted with oxetanyl.

109. The compound of any one of claims 1-108, wherein T² is

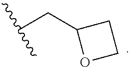 [SMILES: *CC1CCO1]

110. The compound of claim 109, wherein the stereogenic center in 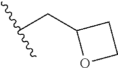 [SMILES: *CC1CCO1]
has (S)-configuration.

111. The compound of any one of claims 1-110, wherein L¹ is (C₁-C₃)alkylene which is optionally substituted with 1-3 R^{L}.

112. The compound of any one of claims 1-111, wherein L¹ is CH₂.

113. The compound of any one of claims 1-110, wherein L¹ is a bond.

114. The compound of any one of claims 1-113, wherein L² is a bond.

115. The compound of any one of claims 1-110, wherein L¹ is CH₂; and L² is a bond.

116. The compound of any one of claims 1-110, wherein L¹ is a bond; and L² is a bond.

117. The compound of any one of claims 1-116, wherein Ring A is

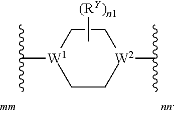

118. The compound of claim 117, wherein W¹ is N.

119. The compound of claim 117 or 118, wherein W² is CR^{Y2}.

120. The compound of claim 119, wherein R² is hydrogen.

121. The compound of claim 117 or 118, wherein W² is N.

122. The compound of any one of claims 117-121, wherein n1 is 0.

123. The compound of any one of claims 117-121, wherein n1 is 1.

124. The compound of any one of claims 1-117, wherein Ring A is

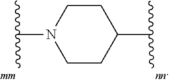 [SMILES: *C1CCN(*)CC1]

125. The compound of any one of claims 1-116, wherein Ring A is

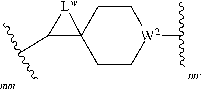

126. The compound of claim 125, wherein L^{w} is CH₂.

127. The compound of claim 125 or 126, wherein W² is N.

128. The compound of any one of claims 1-116 or 125-127, wherein Ring A is

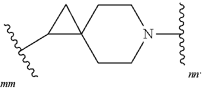 [SMILES: *C1CC12CCN(*)CC2]

129. The compound of any one of claims 1-116, wherein Ring A is selected from the group consisting of:
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y}.

130. The compound of any one of claims 1-116 or 129, wherein Ring A is 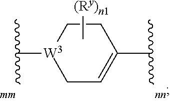
W³ is N or CH; and n1 is 0, 1, or 2.

131. The compound of claim 130, wherein n1 is 0.

132. The compound of claim 130 or 131, wherein W³ is N.

133. The compound of claim 130 or 131, wherein W³ is CH.

134. The compound of any one of claims 1-116 or 130, wherein Ring A is

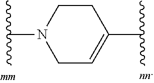 [SMILES: *C1=CCN(*)CC1]

135. The compound of any one of claims 1-116 or 130, wherein Ring A is

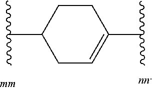 [SMILES: *C1=CCC(*)CC1]

136. The compound of any one of claims 1-110, wherein L² is a bond; L¹ is CH₂; and Ring A is

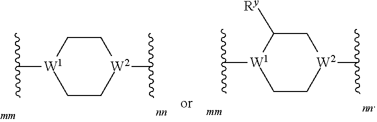

137. The compound of claim 136, wherein Ring A is

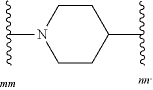 [SMILES: *C1CCN(*)CC1]

138. The compound of any one of claims 1-110, wherein L² is a bond; L¹ is CH₂; and Ring A is

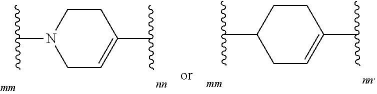 [SMILES: *C1=CCN(*)CC1 ; *C1=CCC(*)CC1]

139. The compound of any one of claims 1-110, wherein L² is a bond; L is a bond; and Ring A is

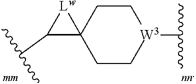

140. The compound of claim 139, wherein Ring A is

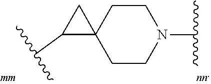 [SMILES: *C1CC12CCN(*)CC2]

141. The compound of claim 138, wherein L² is a bond; L¹ is CH₂, and Ring A is

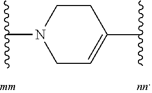 [SMILES: *C1=CCN(*)CC1]

142. The compound of any one of claims 1-110, wherein L² is a bond; L¹ is CH₂; and Ring A is

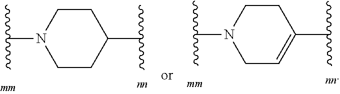 [SMILES: *C1CCN(*)CC1 ; *C1=CCN(*)CC1]

143. The compound of any one of claims 1-142, wherein Ring B is

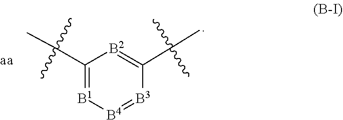

144. The compound of any one of claims 1-143, wherein B⁴ is CR¹.

145. The compound of any one of claims 1-144, wherein B⁴ is CH.

146. The compound of any one of claims 1-145, wherein B¹ is CR¹.

147. The compound of any one of claims 1-146, wherein B is CH.

148. The compound of any one of claims 1-147, wherein B³ is CR¹.

149. The compound of any one of claims 1-148, wherein B¹ is CH.

150. The compound of any one of claims 1-149, wherein B² is N.

151. The compound of any one of claims 1-143, wherein Ring B is

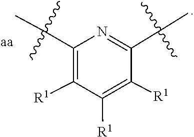

152. The compound of any one of claims 1-143 or 151, wherein Ring B is

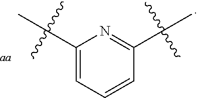 [SMILES: *c1cccc(*)n1]

153. The compound of any one of claims 1-142, wherein Ring B is

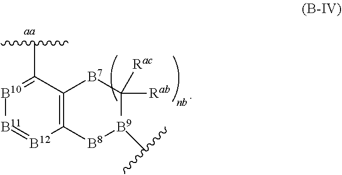

154. The compound of any one of claims 1-142 or 153, wherein Ring B is

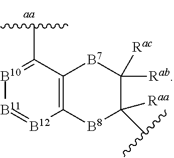

155. The compound of any one of claims 1-142 or 153, wherein Ring B is

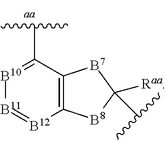

156. The compound of any one of claims 1-142, wherein Ring B is

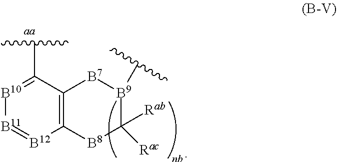

157. The compound of any one of claims 1-142 or 156, wherein Ring B is

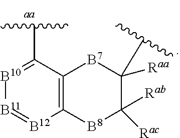

158. The compound of any one of claims 153-157, wherein B⁷ is —O—.

159. The compound of any one of claims 153-158, wherein B⁸ is —O—.

160. The compound of any one of claims 153-159, wherein B⁷ is —O—; and B⁸ is —O—.

161. The compound of any one of claims 153-160, wherein R^{aa} is H.

162. The compound of any one of claims 153-160, wherein R^{aa} is (C₁-C₃)alkyl.

163. The compound of any one of claims 153-160 or 162, wherein R^{aa} is methyl.

164. The compound of any one of claims 153-154 or 156-163, wherein R^{ab} is H.

165. The compound of any one of claims 153-154 or 156-164, wherein R^{ac} is H.

166. The compound of any one of claims 153-154 or 156-160, wherein R^{aa}, R^{ab}, and R^{ac} are each H.

167. The compound of any one of claims 153-154 or 156-160, wherein R^{aa} is (C₁-C₃)alkyl; and R^{ab} and R^{ac} are H.

168. The compound of any one of claims 153-167, wherein B¹⁰ is CR¹.

169. The compound of any one of claims 153-168, wherein B¹⁰ is CH.

170. The compound of any one of claims 153-169, wherein B¹⁰ is CR¹.

171. The compound of any one of claims 153-170, wherein B¹¹ is CH.

172. The compound of any one of claims 153-171, wherein B¹² is CR¹.

173. The compound of any one of claims 153-172, wherein B¹² is CH.

174. The compound of any one of claims 1-142 or 153, wherein Ring B is 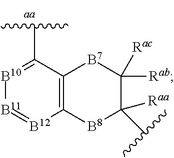
B⁷ and B⁸ are —O—; and R^{aa} is H or (C₁-C₃)alkyl.

175. The compound of any one of claims 1-142 or 153, wherein Ring B is 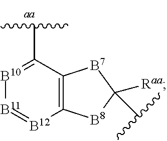
B⁷ and B⁸ are —O—; and R^{aa} is H or (C₁-C₃)alkyl.

176. The compound of any one of claims 1-142 or 156, wherein Ring B is 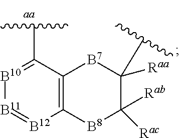
B⁷ and B⁸ are —O—; and R^{aa} is H or (C₁-C₃)alkyl.

177. The compound of any one of claims 174-176, wherein R^{aa} is H.

178. The compound of any one of claims 174-176, wherein R^{aa} is (C₁-C₃)alkyl.

179. The compound of any one of claim 174 or 176-178, wherein R^{ab} and R^{ac} are H.

180. The compound of any one of claims 174-179, wherein B¹⁰, B¹¹, and B¹² are each independently selected CR¹.

181. The compound of any one of claims 174-180, wherein B¹⁰, B, and B¹² are CH.

182. The compound of any one of claims 154-155 or 157-181, wherein the carbon atom to which L⁴ and R^{aa} are both attached has (R)-configuration.

183. The compound of any one of claims 154-155 or 157-181, wherein the carbon atom to which L⁴ and R^{aa} are both attached has (S)-configuration.

184. The compound of any one of claims 1-142, wherein Ring B is 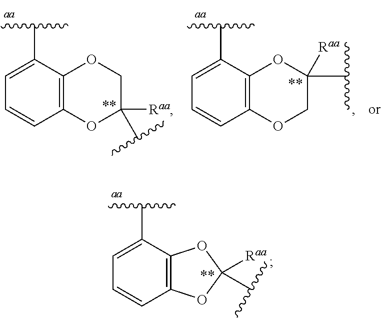
and the carbon atom labelled with ** has (R)-configuration; or wherein Ring B is 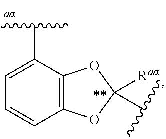
and the carbon atom labelled with ** has (S)-configuration.

185. The compound of any one of claims 1-184, wherein L⁴ is a bond.

186. The compound of any one of claims 153-184, wherein L⁴ is a bond.

187. The compound of any one of claims 1-184, wherein L⁴ is —Z¹—Z²_*, wherein * represents the point of attachment to Ring C.

188. The compound of any one of claims 1-184 or 187, wherein Z¹ is —O—.

189. The compound of any one of claims 1-184 or 187, wherein Z¹ is a bond.

190. The compound of any one of claims 1-184 or 187-189, wherein Z² is —CH₂— optionally substituted with 1-2 R^{c}.

191. The compound of any one of claims 1-184 or 187-190, wherein Z² is —CH₂—.

192. The compound of any one of claims 1-184, wherein L⁴ is —O—CH₂—*, wherein * represents the point of attachment to Ring C.

193. The compound of any one of claims 1-184, wherein L⁴ is —CH₂—.

194. The compound of any one of claims 1-141 or 143-184, wherein L⁴ is a bond.

195. The compound of any one of claims 1-141 or 143-184, wherein; and L⁴ is —O—Z²—*, wherein * represents the point of attachment to Ring C; and Z² is —CH₂-optionally substituted with 1-2 R^{c}, such as wherein Z² is —CH₂—.

196. The compound of any one of claims 1-140 or 142-184, wherein L⁴ is —CH₂—.

197. The compound of any one of claims 1-196, wherein Ring C is selected from the group consisting of: phenyl, 5- to 6-membered heteroaryl, and 5- to 10-membered bicycloheteroaryl.

198. The compound of any one of claims 1-197, wherein Ring C is selected from the group consisting of: phenyl and 6-membered heteroaryl.

199. The compound of any one of claims 1-198, wherein Ring C is phenyl.

200. The compound of any one of claims 1-199, wherein b is 1-3.

201. The compound of any one of claims 1-200, wherein b is 2.

202. The compound of any one of claims 1-200, wherein b is 1.

203. The compound of any one of claims 1-199, wherein b is 0.

204. The compound of any one of claims 1-199, wherein Ring C is phenyl; and b is 2.

205. The compound of any one of claims 1-199 or 204, wherein

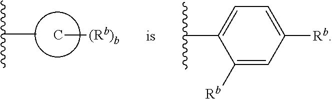

206. The compound of any one of claims 1-199, wherein Ring C is phenyl; and b is 1.

207. The compound of any one of claims 1-199 or 206, wherein

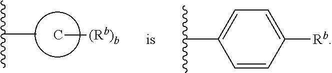

208. The compound of any one of claims 1-199, wherein Ring C is phenyl; and b is 0.

209. The compound of any one of claims 1-207, wherein each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN.

210. The compound of any one of claims 1-207 or 209, wherein each occurrence of R^{b} is independently selected from the group consisting of —F, —Cl, CF₃, and CN.

211. The compound of claim 1, wherein the compound is a compound of Formula (I-A1) or a pharmaceutically acceptable salt thereof: 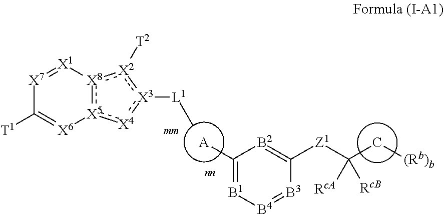
wherein R^{cA} and R^{cB} are independently selected from the group consisting of H and R.

212. The compound of claim 211, wherein Z¹ is —O—.

213. The compound of claim 211 or 212, wherein R^{cA} is H.

214. The compound of any one of claims 211-213, wherein R^{cB} is H.

215. The compound of claim 1, wherein the compound is a compound of Formula (I-A3): 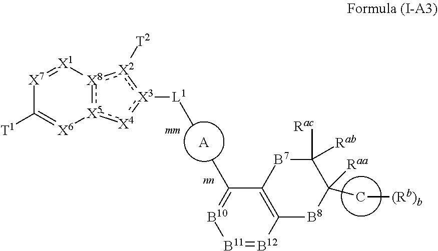
or a pharmaceutically acceptable salt thereof.

216. The compound of claim 1, wherein the compound is a compound of Formula (I-A4): 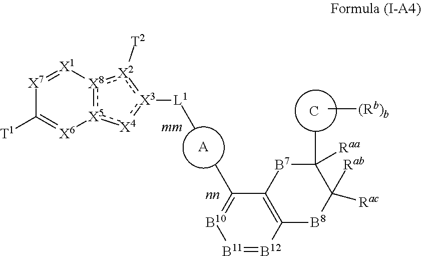
or a pharmaceutically acceptable salt thereof.

217. The compound of claim 215 or 216, wherein R^{ab} and R^{ac} are H.

218. The compound of claim 1, wherein the compound is a compound of Formula (I-A5): 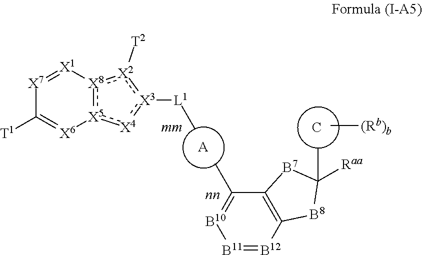
or a pharmaceutically acceptable salt thereof.

219. The compound of any one of claims 215-218, wherein R^{aa} is H.

220. The compound of any one of claims 215-218, wherein R^{aa} is (C₁-C₃)alkyl.

221. The compound of any one of claims 215-218 or 220, wherein R^{aa} is methyl.

222. The compound of any one of claims 215-221, wherein B⁷ is —O—; and B, is —O—.

223. The compound of any one of claims 215-222, wherein B¹⁰, B¹¹, and B¹² are independently selected CR¹, such as wherein B¹⁰, B¹¹, and B¹² are each CH.

224. The compound of any one of claims 215-223, wherein the carbon to which both R^{aa} and Ring C are attached has (R)-configuration.

225. The compound of any one of claims 215-223, wherein the carbon to which both R^{aa} and Ring C are attached has (S)-configuration.

226. The compound of any one of claims 211-225, wherein X³ is C; X² and X⁴ are N; and X⁵ and X⁸ are C.

227. The compound of any one of claims 211-226, wherein the 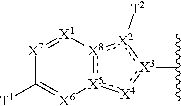
moiety is

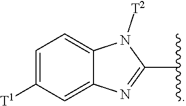

228. The compound of any one of claims 211-226, wherein the 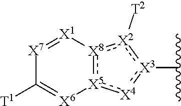
moiety is

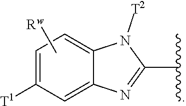

229. The compound of any one of claims 211-226, wherein the 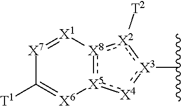
moiety is

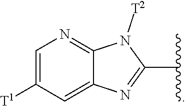

230. The compound of any one of claims 211-226, wherein the 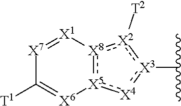
moiety is

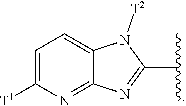

231. The compound of any one of claims 211-226, wherein the 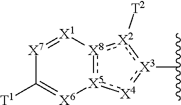
moiety is

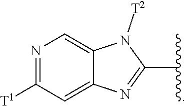

232. The compound of any one of claims 211-231, wherein T¹ is —NR^{s}C(═O)R^{z} or —NR^{s}S(O)₁₋₂—R^{z}; and R^{z} is (C₁-C₆)alkyl optionally substituted with 1-4 R^{c}.

233. The compound of claim 232, wherein R^{z} is (C₁-C₃)alkyl.

234. The compound of claim 232, wherein R^{z} is (C₁-C₃)alkyl substituted with (C₁-C₃)alkoxy; or wherein R^{z} is (C₁-C₃)alkyl substituted with 1-3 independently selected halo, such as wherein R^{z} is —CH₂CF₃.

235. The compound of any one of claims 211-231, wherein T¹ is —NR^{s}C(═O)R^{z} or —NR^{s}S(O)₁₋₂—R^{z}; and R^{z} is —R^{z3} or —CH₂—R^{z3}.

236. The compound of claim 235, wherein R^{z3} is selected from the group consisting of: phenyl, (C₃-C₆)cycloalkyl, and 3- to 6-membered heterocycloalkyl, each optionally substituted with 1-2 R^{v}, such as wherein R^{z3} is phenyl, cyclopropyl, cyclobutyl, difluorocyclobutyl, cyclopentyl, oxetanyl, tetrahydropyranyl, or tetrahydro-2H-thiopyran 1,1-dioxide.

237. The compound of any one of claims 232-236, wherein T¹ is —NHC(═O)R^{z}.

238. The compound of any one of claims 232-236, wherein T¹ is —NHS(O)₂—R^{z}.

239. The compound of any one of claims 211-231, wherein T¹ is —(CR^{x}R^{x})_{q} S(O)₂R^{z}; and q is 1, 2, or 3.

240. The compound of claim 239, wherein R^{z} is (C₁-C₆)alkyl optionally substituted with 1-4 R^{v}.

241. The compound of claim 239 or 240, wherein R^{z} is (C₁-C₃)alkyl.

242. The compound of any one of claims 239-241, wherein q is 1.

243. The compound of any one of claims 239-242, wherein each R^{x} is hydrogen.

244. The compound of any one of claims 211-231, wherein T¹ is —(CR^{x}R^{x})_{q}—C(═O)OH; and q is 1, 2, or 3.

245. The compound of claim 244, wherein q is 1.

246. The compound of claim 244, wherein q is 2.

247. The compound of any one of claims 244-2462, wherein each R^{x} is hydrogen.

248. The compound of any one of claims 244-246, wherein one pair of R^{x} on the same carbon, taken together with the carbon atom to which each is attached, forms a (C₃-C₈)cycloalkyl ring; and each remaining R^{x} when present is hydrogen.

249. The compound of claim 248, wherein the pair of R^{x} on the same carbon, taken together with the carbon atom to which each is attached, forms a cyclopropyl ring.

250. The compound of any one of claims 211-231 or 244, wherein T¹ is

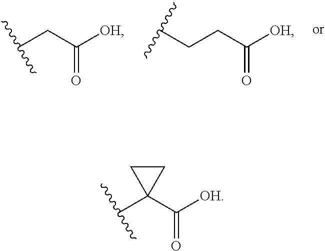 [SMILES: *CC(=O)O ; *CCC(=O)O ; "or" ; *C1(C(=O)O)CC1]

251. The compound of any one of claims 211-231, wherein T¹ is —O—(CR^{x}R^{x})_{q}—C(═O)OH; and q is 1, 2, or 3.

252. The compound of claim 251, wherein q is 1.

253. The compound of claim 251 or 252, wherein each R^{x} is hydrogen.

254. The compound of any one of claims 211-231, wherein T¹ is —P(═O)R^{z1}R^{z2}.

255. The compound of claim 254, wherein R^{z1} and R^{z2} are independently selected (C₁-C₃)alkyl.

256. The compound of any one of claims 211-231, wherein T¹ is 5-membered heteroaryl having 2-4 ring heteroatoms selected from the group consisting of N, O, and S, wherein the heteroaryl is optionally substituted with 1-2 R^{v}.

257. The compound of any one of claims 211-231 or 256, wherein T¹ is selected from the group consisting of:

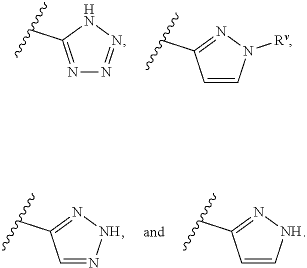

258. The compound of any one of claims 211-231, wherein T¹ is 5- to 6-membered heteroaryl that comprises an endocyclic group selected from the group consisting of: 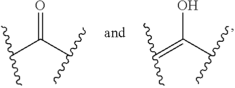
wherein the heteroaryl is further optionally substituted with 1-4 R^{v}.

259. The compound of any one of claims 211-237 or 264, wherein T¹ is selected from the group consisting of:

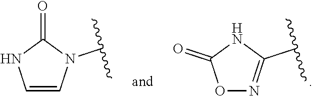 [SMILES: *n1cc[nH]c1=O ; *c1noc(=O)[nH]1]

260. The compound of any one of claims 211-231, wherein T¹ is 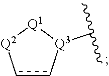
Q¹ is C(═O) or S(O)₂; Q² is O, NH, —CH₂—, or —CH₂—CH₂—; Q³ is N or CH; and [symbol] is a single bond or a double bond, provided that T³ is non-aromatic.

261. The compound of any one of claims 211-231 or 260, wherein T¹ is selected from the group consisting of:

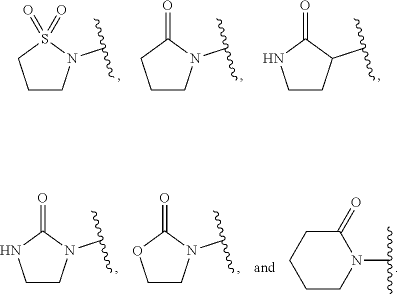

262. The compound of any one of claims 211-261, wherein T² is (C₁-C₆)alkyl which is substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.

263. The compound of any one of claims 211-262, wherein T² is (C₁-C₃)alkyl which is substituted with 4- to 6-membered heterocycloalkyl.

264. The compound of any one of claims 211-263, wherein T² is (C₁-C₃)alkyl which is substituted with oxetanyl.

265. The compound of any one of claims 211-264, wherein T² is 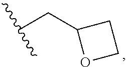 [SMILES: *CC1CCO1]
optionally wherein the stereogenic center in T² has (S)-configuration.

266. The compound of any one of claims 211-265, wherein L¹ is CH₂; and Ring A is

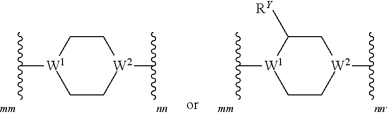

267. The compound of any one of claims 211-266, wherein Ring A is

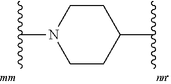 [SMILES: *C1CCN(*)CC1]

268. The compound of any one of claims 211-265, wherein L¹ is a bond; and Ring A is

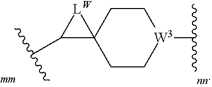

269. The compound of any one of claims 211-265 or 268, wherein Ring A is

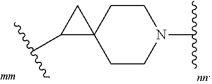 [SMILES: *C1CC12CCN(*)CC2]

270. The compound of any one of claims 211-265, wherein L¹ is CH₂; and Ring A is 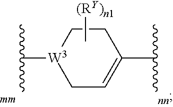
W³ is N or CH; and n1 is 0, 1, or 2.

271. The compound of any one of claims 211-265 or 270, wherein Ring A is

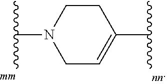 [SMILES: *C1=CCN(*)CC1]

272. The compound of any one of claims 211-265 or 270, wherein Ring A is

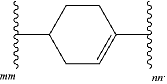 [SMILES: *C1=CCC(*)CC1]

273. The compound of any one of claims 211-272, wherein Ring C is selected from the group consisting of: phenyl and 6-membered heteroaryl; and b is 1 or 2.

274. The compound of any one of claims 211-273, wherein

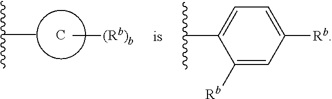

275. The compound of any one of claims 211-273, wherein

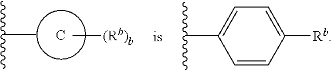

276. The compound of any one of claims 211-273, wherein

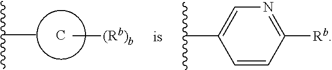

277. The compound of any one of claims 211-276, wherein each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN.

278. The compound of any one of claims 211-277, wherein each occurrence of R^{b} is independently selected from the group consisting of —F, —Cl, CF₃, and CN.

279. The compound of any one of claims 1-278, wherein the compound of Formula I is selected from the group consisting of the compounds in Table C₁ or Table C₂, or a pharmaceutically acceptable salt or solvate thereof.

280. A pharmaceutical composition comprising a compound of any one of claims 1-279, or a pharmaceutically acceptable salt or solvate thereof, and a pharmaceutically acceptable excipient.

281. A method of treating type 2 diabetes mellitus in a patient in need thereof, the method comprising administering to the patient a therapeutically effective amount of a compound of any one of claims 1-279, or a pharmaceutically acceptable salt or solvate thereof, or a pharmaceutical composition according to claim 286.

282. A method for treating type 2 diabetes mellitus in a patient, the method comprising administering to a patient identified or diagnosed as having type 2 diabetes mellitus a therapeutically effective amount of a compound of any one of claims 1-279, or a pharmaceutically acceptable salt or solvate thereof, or a pharmaceutical composition according to claim 280.

283. A method of treating diabetes mellitus in a patient, the method comprising:
a) determining that the patient has type 2 diabetes mellitus; and
b) administering to the patient a therapeutically effective amount of a compound of any one of claims 1-279, or a pharmaceutically acceptable salt or solvate thereof, or a pharmaceutical composition according to claim 280.

284. The method of any one of claims 287-283, wherein the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof.

285. The method of claim 284, wherein the level of HbA1c is greater than or about 6.5%.

286. The method of claim 284 or 285, wherein the level of fasting plasma glucose is greater than or about 126 mg/dL.

287. The method of claim 284 or 285, wherein the level of non-fasting plasma glucose is greater than or about 200 mg/dL.

288. The method of any one of claims 281-287, further comprising obtaining a sample from the patient.

289. The method of claim 288, wherein the sample is a body fluid sample.

290. The method of any one of claims 281-289, wherein the patient is about 40 to about 70 years old and is overweight or obese.

291. The method of any one of claims 281-290, wherein the patient has a body mass index (BMI) greater than or about 22 kg/m².

292. The method of any one of claims 281-291, wherein the patient has a BMI greater than or about 30 kg/m².

293. The method of any one of claims 281-292, wherein the treatment of type 2 diabetes mellitus comprises a reduction in fasting plasma glucose levels.

294. The method of claim 293, wherein the fasting plasma glucose levels are reduced to about or below 100 mg/dL.

295. The method of any one of claims 281-294, wherein the treatment of type 2 diabetes mellitus comprises a reduction in HbA1c levels.

296. The method of claim 295, wherein the HbA1c levels are reduced to about or below 5.7%.

297. The method of any one of claims 281-296, wherein the treatment of type 2 diabetes mellitus comprises a reduction in glucagon levels.

298. The method of any one of claims 281-297, wherein the treatment of type 2 diabetes mellitus comprises an increase in insulin levels.

299. The method of any one of claims 281-298, wherein the treatment of type 2 diabetes mellitus comprises a decrease in BMI.

300. The method of claim 299, wherein the BMI is decreased to about or below 25 kg/m².

301. The method of any of one of claims 281-300, wherein the compound of any one of claims 1-285, or a pharmaceutically acceptable salt or solvate thereof, or a pharmaceutical composition according to claim 286, is administered orally.

302. The method of any one of claims 281-301, further comprising administering an additional therapy or therapeutic agent to the patient.

303. The method of claim 302, wherein the additional therapy or therapeutic agent is selected from the group consisting of an antidiabetic agent, an anti-obesity agent, a GLP-1 receptor agonist, an agent to treat non-alcoholic steatohepatitis (NASH), an anti-emetic agent, gastric electrical stimulation, dietary monitoring, physical activity, or any combinations thereof.

304. The method of claim 303, wherein the antidiabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof.

305. The method of claim 304, wherein the biguanide is metformin.

306. The method of claim 303, wherein the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, or any combinations thereof.

307. The method of claim 303, wherein the GLP-1 receptor agonist is selected from the group consisting of liraglutide, exenatide, dulaglutide, albiglutide, taspoglutide, lixisenatide, semaglutide, or any combinations thereof.

308. The method of claim 303, wherein the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASK1) inhibitor, or any combinations thereof.

309. The method of any one of claims 302-308, wherein the compound of any one of claims 1-285 or a pharmaceutically acceptable salt or solvate thereof, or a pharmaceutical composition according to claim 286, and the additional therapeutic agent are administered as separate dosages sequentially in any order.

310. A method for modulating insulin levels in a patient in need of such modulating, the method comprising administering to the patient an effective amount of a compound as claimed in any one of claims 1-279, or a pharmaceutically acceptable salt or solvate thereof, or a pharmaceutical composition according to claim 280.

311. The method of claim 310, wherein the modulation results in an increase of insulin levels.

312. A method for modulating glucose levels in a patient in need of such modulating, the method comprising administering to the patient an effective amount of a compound as claimed in any one of claims 1-279, or a pharmaceutically acceptable salt or solvate thereof, or a pharmaceutical composition according to claim 280.

313. The method of claim 312, wherein the modulation results in a decrease of glucose levels.

314. A method for treating a GLP-1 associated disease, disorder, or condition, the method comprising administering to a patient in need thereof an effective amount of a compound as claimed in any one of claims 1-279, or a pharmaceutically acceptable salt or solvate thereof, or a pharmaceutical composition according to claim 280.

315. The method of claim 314, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof.

316. The method of claim 315, wherein the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

317. The method of claim 316, wherein the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.
